(19) United States
(12) Patent Application Publication
(10) Pub. No.: US 2025/0082648 A1
(43) Pub. Date: Mar. 13, 2025

(54) COMPOSITIONS AND METHODS FOR ENHANCING KRAS INHIBITOR OR SHP2 INHIBITOR EFFICACY

(71) Applicant: New York University, New York, NY (US)

(72) Inventors: Benjamin G. NEEL, New York, NY (US); Kwok-Kin WONG, New York, NY (US); Suman MUKHOPADHYAY, Astoria, NY (US); Wei WEI, New York, NY (US)

(73) Assignee: New York University, New York, NY (US)

(21) Appl. No.: 18/826,792

(22) Filed: Sep. 6, 2024

Related U.S. Application Data
(60) Provisional application No. 63/581,469, filed on Sep. 8, 2023.

(51) Int. Cl. A61K 31/551; A61K 31/415; A61K 31/4439; A61K 31/4709; A61K 31/496; A61K 31/4985; A61K 31/517; A61K 31/519; A61K 31/5377; A61K 35/17; A61K 39/00; A61P 35/00

(52) U.S. Cl. CPC A61K 31/551; A61K 31/415; A61K 31/4439; A61K 31/4709; A61K 31/496; A61K 31/4985; A61K 31/517; A61K 31/519; A61K 31/5377; A61K 35/17; A61K 40/11; A61K 40/15; A61K 40/31; A61K 40/4253; A61P 35/00

(57) ABSTRACT

This application relates to methods for overcoming or preventing resistance of a KRAS mutant cancer cell to a growth inhibition and/or cell death induction by a KRAS inhibitor or a SHP2 inhibitor, as well as to methods for enhancing sensitivity of a KRAS mutant cancer cell to a KRAS inhibitor or a SHP2 inhibitor. The application further relates to methods of treating a KRAS mutant cancer in a subject, the method comprising administering to the subject an effective amount of a KRAS inhibitor or a SHP2 inhibitor and an inhibitor of expression or function or a degrader or a binding partner of one or more of various proteins described herein. Related pharmaceutical compositions and kits are also disclosed.

### CROSS-REFERENCE TO RELATED APPLICATION

This patent application claims the benefit of U.S. Provisional Application No. 63/581,469, filed Sep. 8, 2023, the disclosure of which is incorporated by reference herein in its entirety for all purposes.

### STATEMENT REGARDING FEDERALLY SPONSORED RESEARCH

This invention was made with government support under CA248896 and CA016087 awarded by National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Aug. 26, 2024, is named 243735_000389_SL.xml and is 2,631 bytes in size.

### FIELD OF THE INVENTION

This application relates to methods for overcoming or preventing resistance of a KRAS mutant cancer cell to a growth inhibition and/or cell death induction by a KRAS inhibitor or a SHP2 inhibitor, as well as to methods for enhancing sensitivity of a KRAS mutant cancer cell to a KRAS inhibitor or a SHP2 inhibitor. The application further relates to methods of treating a KRAS mutant cancer in a subject, the method comprising administering to the subject an effective amount of a KRAS inhibitor or a SHP2 inhibitor and an inhibitor of expression or function or a degrader or a binding partner of one or more of various proteins described herein. Related pharmaceutical compositions and kits are also disclosed.

### BACKGROUND

The most common subtype of lung cancer, non-small cell lung cancer (NSCLC) is a leading cause of cancer-associated morbidity and mortality worldwide. Mutations in Kirsten Rat Sarcoma Viral Oncogene Homolog (KRAS) drive 25-30% of NSCLC cases (1-3); approximately half of these mutations convert glycine 12 to cysteine (G12C). Concomitant mutation or deletion (hereinafter, “co-mutations”) of different tumor suppressor genes (e.g., TP53, SMARCA4, STK11, and/or KEAP1) typically occur in concert with KRAS mutations (4,5). KRAS mutations also occur frequently in other tumors, including colorectal cancer (CRC) and pancreatic adenocarcinoma (PDAC). The specific KRAS allele, as well as other co-occurring genomic abnormalities, differ in these neoplasms, with KRAS^{G12N} comprising only 25% of CRC-associated KRAS mutations (6% of CRC overall) and 1-3% of PDAC cases. Tumor properties, including therapy response, are determined by the specific combination of driver and tumor suppressor gene alterations and the cell-of-origin of the tumor. In NSCLC, for example, STK11 and/or KEAP1 mutations have been associated with poor response to conventional, targeted, and immune therapies (5-7). Effective treatment strategies for this subgroup are a major unmet medical need.

KRAS had long been viewed as “undruggable”. The recent development of small molecule covalent G12C inhibitors (G12Cis) represents a triumph of chemical biology and drug design. Several G12Cis (6,8) are in clinical trials and two, sotorasib (AMG-510) and adagrasib (MRTX-849), are now FDA-approved for second-line treatment of KRAS^{G12C} NSCLC. While these drugs clearly have clinical activity, overall response rates (30-40%) and disease control (˜60%) in NSCLC are modest and transient (median duration approximately under a year) (9,10). Response rates in KRAS^{G12C}-mutant CRC are even lower (11).

Multiple mechanisms of intrinsic and acquired resistance (10,12,13) have been identified, and combination strategies almost certainly will be needed to maximize the clinical efficacy of G12Cis in NSCLC and other diseases. For example, combinations of G12Ci and SHP2 inhibitors (SHP2i) such TNO155 (14) and RMC-4550 (15) have already been validated pre-clinically and are now being explored in the clinic (NCT04699188, NCT05480865).

### SUMMARY OF THE INVENTION

As specified in the Background section above, there is a great need in the art for identification of druggable targets for use in combination with KRAS inhibitors (e.g., KRAS^{G12C} inhibitors) and SHP2 inhibitors for the treatment of cancers such as lung cancer (e.g., non-small cell lung cancer). The present application addresses these and other needs.

In one aspect, provided herein is a method for overcoming or preventing resistance of a KRAS mutant cancer cell to a growth inhibition and/or cell death induction by a KRAS inhibitor, comprising inhibiting in the KRAS mutant cancer cell expression or function of one or more proteins selected from VRK1, RIOK2, EXT1, EXT2, ELP2, ELP3, ELP5, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2.

In one aspect, provided herein is a method for enhancing sensitivity of a KRAS mutant cancer cell to a KRAS inhibitor, comprising inhibiting in the KRAS mutant cancer cell expression or function of one or more proteins selected from VRK1, RIOK2, EXT1, EXT2, ELP2, ELP3, ELP5, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2.

In some embodiments, the method comprises inhibiting in said KRAS mutant cancer cell expression or function of one or more proteins selected from TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and LARS2.

In some embodiments, the method comprises inhibiting in said KRAS mutant cancer cell expression or function of one or more proteins selected from AP2S1, ERBB3, GRB2, CRK, SRC, PAK2, EXT2, NDST1, SHOC2, and IPO11.

In some embodiments, the method comprises inhibiting in said KRAS mutant cancer cell expression or function of one or more proteins selected from YAP, WWTR1, and TEAD.

In some embodiments, the method comprises inhibiting in said KRAS mutant cancer cell expression or function MTOR protein and/or RPTOR protein.

In some embodiments, the method comprises inhibiting in the KRAS mutant cancer cell expression or function of one or more proteins selected from VRK1, ELP2, ELP3, PKN2, RIOK2, EXT1, and EXT2.

In some embodiments, the method comprises inhibiting in the KRAS mutant cancer cell expression or function of VRK1 protein, PKN2 protein, and/or RIOK2 protein.

In some embodiments, the method comprises inhibiting in the KRAS mutant cancer cell kinase activity of VRK1 protein, RIOK2 protein, and/or PKN2 protein.

In some embodiments, the method comprises inhibiting in the KRAS mutant cancer cell ATPase activity of RIOK2 protein.

In some embodiments, the method comprises administering to the KRAS mutant cancer cell an inhibitor of expression or function of the one or more proteins or a degrader of the one or more proteins.

In some embodiments, the inhibitor of expression or function of the one or more proteins or the degrader of the one or more proteins is administered to the KRAS mutant cancer cell simultaneously or sequentially with the KRAS inhibitor.

In some embodiments, the method comprises administering to the KRAS mutant cancer cell an inhibitor of expression or function or degrader of ROCK1 protein and/or ROCK2 protein.

In some embodiments, the inhibitor of expression or function or degrader of ROCK1 protein and/or ROCK2 protein is selected from AR-12286, Fasudil, Ripasudil, Netarsudil, KD025 (Belumosudil), AT13148, GSK269962, H1152, GSK429286, pharmaceutically acceptable salts thereof, and any combinations thereof.

In some embodiments, the KRAS mutant cancer cell comprises a mutation selected from a KRAS G12 mutation, a KRAS G13 mutation, a KRAS H61 mutation, and a KRAS K117 mutation.

In some embodiments, the KRAS mutant cancer cell comprises a KRAS G12 mutation selected from G12C, G12V, G12D, G12S, and G12R mutation.

In some embodiments, the KRAS mutant cancer cell comprises the KRAS G12C mutation.

In some embodiments, the KRAS inhibitor is a KRAS G12C inhibitor (G12Ci).

In some embodiments, the KRAS mutant cancer cell comprises the KRAS G13D mutation.

In some embodiments, the KRAS mutant cancer cell comprises a KRAS H61 mutation selected from Q61H, Q61L, and Q61R mutation.

In some embodiments, the KRAS mutant cancer cell comprises the KRAS K117N mutation.

In some embodiments, the KRAS mutant cancer cell also has a mutation in STK11 (LKB1) gene and/or KEAP1 gene.

In some embodiments, the KRAS mutant cancer cell also has deletion or reduced expression of one or more genes associated with and/or predictive of resistance of the KRAS mutant cancer cell to treatment with the KRAS inhibitor. In certain embodiments, the one or more genes is selected from, e.g., SPRED1, SPRED2, NF1, SPYR2, AMOTL2, LATS1, LATS2, KIRREL, NF2, PTPN14, PTEN, TSC1, and TSC2.

In some embodiments, the KRAS mutant cancer cell is in a subject.

In some embodiments, the KRAS inhibitor is selected from adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some embodiments, the KRAS inhibitor is KRAS G12C inhibitor (G12Ci) adagrasib (MRTX-849) or sotorasib (AMG510).

In one aspect, provided herein is a method of treating a KRAS mutant cancer in a subject in need thereof, comprising administering to the subject an effective amount of a KRAS inhibitor and an inhibitor of expression or function or a degrader of one or more proteins selected from VRK1, RIOK2, PKN2, EXT1, EXT2, ELP2, ELP3, ELP5, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2.

In some embodiments, the method comprises administering to the subject an effective amount of a KRAS inhibitor and an inhibitor of expression or function or a degrader of one or more proteins selected from TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and LARS2.

In some embodiments, the method comprises administering to the subject an effective amount of a KRAS inhibitor and an inhibitor of expression or function or a degrader of one or more proteins selected from AP2S1, ERBB3, GRB2, CRK, SRC, PAK2, EXT2, NDST1, SHOC2, and IPO11.

In some embodiments, the method comprises administering to the subject an effective amount of a KRAS inhibitor and an inhibitor of expression or function or a degrader of one or more proteins selected from YAP, WWTR1, and TEAD.

In some embodiments, the method comprises administering to the subject an effective amount of a KRAS inhibitor and an inhibitor of expression or function or a degrader of MTOR protein and/or RPTOR protein.

In some embodiments, the method comprises administering to the subject an effective amount of a KRAS inhibitor and an inhibitor of expression or function or a degrader of one or more proteins selected from VRK1, PKN2, ELP2, ELP3, RIOK2, EXT1, and EXT2.

In some embodiments, the method comprises administering to the subject an effective amount of a KRAS inhibitor and an inhibitor of expression or function or a degrader of VRK1 protein, PKN2 protein, and/or RIOK2 protein.

In some embodiments, the method comprises administering to the subject an inhibitor of kinase activity of VRK1 protein, PKN2 protein, and/or RIOK2 protein.

In some embodiments, the method comprises administering to the subject an inhibitor of ATPase activity of RIOK2 protein.

In some embodiments, the KRAS inhibitor and the inhibitor of expression or function or degrader of the one or more proteins are administered simultaneously.

In some embodiments, the KRAS inhibitor and the inhibitor of expression or function or degrader of the one or more proteins are administered simultaneously in one composition.

In some embodiments, the KRAS inhibitor and the inhibitor of expression or function or degrader of the one or more proteins are administered simultaneously in different compositions.

In some embodiments, the KRAS inhibitor and the inhibitor of expression or function or degrader of the one or more proteins are administered sequentially.

In some embodiments, the KRAS inhibitor and/or the inhibitor of expression or function or degrader of the one or more proteins is administered orally or intravenously.

In some embodiments, the method comprises administering an inhibitor of expression or function or degrader of ROCK1 protein and/or ROCK2 protein.

In some embodiments, the inhibitor of expression or function or degrader of ROCK1 protein and/or ROCK2 protein is selected from AR-12286, Fasudil, Ripasudil, Netarsudil, KD025 (Belumosudil), AT13148, GSK269962, H1152, GSK429286, pharmaceutically acceptable salts thereof, and any combinations thereof.

In some embodiments, the KRAS mutant cancer comprises a mutation selected from a KRAS G12 mutation, a KRAS G13 mutation, a KRAS H61 mutation, and a KRAS K117 mutation.

In some embodiments, the KRAS mutant cancer comprises a KRAS G12 mutation selected from G12C, G12V, G12D, G12S, and G12R mutation.

In some embodiments, the KRAS mutant cancer comprises the KRAS G12C mutation.

In some embodiments, the KRAS inhibitor is a KRAS G12C inhibitor (G12Ci).

In some embodiments, the KRAS mutant cancer comprises the KRAS G13D mutation.

In some embodiments, the KRAS mutant cancer comprises a KRAS H61 mutation selected from Q61H, Q61L, and Q61R mutation.

In some embodiments, the KRAS mutant cancer comprises the KRAS K117N mutation.

In some embodiments, the KRAS mutant cancer also has a mutation in STK11 (LKB1) gene and/or KEAP1 gene.

In some embodiments, the KRAS mutant cancer also has deletion or reduced expression of one or more genes associated with and/or predictive of resistance of the KRAS mutant cancer to treatment with the KRAS inhibitor. In certain embodiments, the one or more genes is selected from, e.g., SPRED1, SPRED2, NF1, SPYR2, AMOTL2, LATS1, LATS2, KIRREL, NF2, PTPN14, PTEN, TSC1, and TSC2.

In some embodiments, the KRAS mutant cancer is a lung cancer, colorectal cancer, or pancreatic cancer.

In some embodiments, the KRAS mutant lung cancer is a KRAS mutant non-small cell lung cancer.

In some embodiments, the KRAS mutant cancer is resistant to a treatment with the KRAS inhibitor when the KRAS inhibitor is administered in the absence of the inhibitor of expression or function or degrader of the one or more proteins.

In some embodiments, the subject is human.

In some embodiments, the subject is a veterinary animal.

In some embodiments, the KRAS inhibitor is selected from adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some embodiments, the KRAS inhibitor is the KRAS G12C inhibitor (G12Ci) adagrasib (MRTX-849) or sotorasib (AMG510).

In one aspect, provided herein is a pharmaceutical composition comprising (i) a KRAS inhibitor, (ii) an inhibitor of expression or function or a degrader of one or more proteins selected from VRK1, RIOK2, PKN2, EXT1, EXT2, ELP2, ELP3, ELP5, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2, and (iii) a pharmaceutically acceptable carrier and/or excipient.

In some embodiments, the KRAS inhibitor is a KRAS G12C inhibitor (G12Ci).

In one aspect, provided herein is a kit comprising (i) a KRAS inhibitor, (ii) an inhibitor of expression or function or a degrader of one or more proteins selected from VRK1, RIOK2, PKN2, EXT1, EXT2, ELP2, ELP3, ELP5, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2, and (iii) optionally, instructions for use.

In some embodiments, the KRAS inhibitor is a KRAS G12C inhibitor (G12Ci).

In one aspect, provided herein is a method for overcoming or preventing resistance of a KRAS mutant cancer cell to a growth inhibition and/or cell death induction by a SHP2 inhibitor, comprising inhibiting in the KRAS mutant cancer cell expression or function of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In one aspect, provided herein is a method for enhancing sensitivity of a KRAS mutant cancer cell to a SHP2 inhibitor, comprising inhibiting in the KRAS mutant cancer cell expression or function of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the method comprises inhibiting in the KRAS mutant cancer cell expression or function of one or more proteins selected from VRK1, RIOK2, ELP4, and ELP5.

In some embodiments, the method comprises inhibiting in the KRAS mutant cancer cell function of one or more proteins selected from TFRC, SLC7A6OS, GNB2L1, GFER, ATP6AP2, SLC25A19, and PEAR1.

In one aspect, provided herein is a method for overcoming or preventing resistance of a KRAS mutant cancer cell to a growth inhibition and/or cell death induction by a SHP2 inhibitor, comprising binding on the surface of the KRAS mutant cancer cell one or more proteins selected from TFRC, SLC7A6OS, GNB2L1, GFER, ATP6AP2, SLC25A19, and PEAR1 with a binding partner, wherein the binding partner specifically binds to the one or more proteins.

In one aspect, provided herein is a method for enhancing sensitivity of a KRAS mutant cancer cell to a SHP2 inhibitor, comprising binding on the surface of the KRAS mutant cancer cell one or more proteins selected from TFRC, SLC7A6OS, GNB2L1, GFER, ATP6AP2, SLC25A19, and PEAR1 with a binding partner, wherein the binding partner specifically binds to the one or more proteins.

In some embodiments, the binding partner is capable of inhibiting in the KRAS mutant cancer cell function of one or more proteins selected from TFRC, SLC7A6OS, GNB2L1, GFER, ATP6AP2, SLC25A19, and PEAR1.

In some embodiments, the method comprises administering to the KRAS mutant cancer cell an inhibitor of expression or function of the one or more proteins or a degrader of the one or more proteins.

In some embodiments, the inhibitor of expression or function of the one or more proteins or the degrader of the one or more proteins is administered to the KRAS mutant cancer cell simultaneously or sequentially with the SHP2 inhibitor.

In some embodiments, the binding partner of the one or more proteins is administered to the KRAS mutant cancer cell.

In some embodiments, the binding partner of the one or more proteins is administered to the KRAS mutant cancer cell simultaneously or sequentially with the SHP2 inhibitor.

In some embodiments, the binding partner of the one or more proteins comprises an intact antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a single-chain variable fragment (scFv), a Bi-specific T-cell engager (BiTE), a bispecific killer cell engager (BiKE), a CrossMab, a tri-specific binding partner, or a chimeric antigen receptor (CAR).

In some embodiments, the binding partner of the one or more proteins is conjugated to a detectable label, a chemotherapeutic agent, a radioisotope, or a toxin.

In some embodiments, the binding partner of the one or more proteins is a component of a fusion protein.

In some embodiments, the binding partner of the one or more proteins comprises a chimeric antigen receptor (CAR).

In some embodiments, the binding partner of the one or more proteins is expressed by a T cell or a natural killer cell.

In some embodiments, the KRAS mutant cancer cell comprises a mutation selected from a KRAS G12 mutation, a KRAS G13 mutation, a KRAS H61 mutation, and a KRAS K117 mutation.

In some embodiments, the KRAS mutant cancer cell comprises a KRAS G12 mutation selected from G12C, G12V, G12D, G12S, and G12R mutation.

In some embodiments, the KRAS mutant cancer cell comprises the KRAS G12C mutation.

In some embodiments, the KRAS mutant cancer cell comprises the KRAS G13D mutation.

In some embodiments, the KRAS mutant cancer cell comprises a KRAS H61 mutation selected from Q61H, Q61L, and Q61R mutation.

In some embodiments, the KRAS mutant cancer cell comprises the KRAS K117N mutation.

In some embodiments, the KRAS mutant cancer cell also has a mutation in STK11 (LKB1) gene and/or KEAP1 gene.

In some embodiments, the KRAS mutant cancer cell is in a subject.

In some embodiments, the SHP2 inhibitor is selected from BBP-398, TNO155, RMC-4630, JAB-3068, RLY-1971, and ERAS-601, pharmaceutically acceptable salts thereof, and any combinations thereof.

In one aspect, provided herein is a method of treating a KRAS mutant cancer in a subject in need thereof, comprising administering to the subject an effective amount of a SHP2 inhibitor and an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the method comprises administering to the subject an effective amount of a SHP2 inhibitor and an inhibitor of expression or function or a degrader of one or more proteins selected from VRK1, RIOK2, ELP4, and ELP5.

In some embodiments, the method comprises administering to the subject an effective amount of a SHP2 inhibitor and an inhibitor of function of one or more proteins selected from TFRC, SLC7A6OS, GNB2L1, GFER, ATP6AP2, SLC25A19, and PEAR1.

In some embodiments, the SHP2 inhibitor and the inhibitor of expression or function or degrader of the one or more proteins are administered simultaneously.

In some embodiments, the SHP2 inhibitor and the inhibitor of expression or function or degrader of the one or more proteins are administered simultaneously in one composition.

In some embodiments, the SHP2 inhibitor and the inhibitor of expression or function or degrader of the one or more proteins are administered simultaneously in different compositions.

In some embodiments, the SHP2 inhibitor and the inhibitor of expression or function or degrader of the one or more proteins are administered sequentially.

In some embodiments, the SHP2 inhibitor and/or the inhibitor of expression or function or degrader of the one or more proteins is administered orally or intravenously.

In another aspect, provided herein is a method of treating a KRAS mutant cancer in a subject in need thereof, comprising administering to the subject an effective amount of a SHP2 inhibitor and a binding partner, wherein the binding partner specifically binds to one or more proteins selected from TFRC, SLC7A6OS, GNB2L1, GFER, ATP6AP2, SLC25A19, and PEAR1.

In some embodiments, the SHP2 inhibitor and the binding partner of the one or more proteins are administered simultaneously.

In some embodiments, the SHP2 inhibitor and the binding partner of the one or more proteins are administered simultaneously in one composition.

In some embodiments, the SHP2 inhibitor and the binding partner of the one or more proteins are administered simultaneously in different compositions.

In some embodiments, the SHP2 inhibitor and the binding partner of the one or more proteins are administered sequentially.

In some embodiments, the SHP2 inhibitor and/or the binding partner of the one or more proteins is administered orally or intravenously.

In some embodiments, the binding partner of the one or more proteins comprises an intact antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a single-chain variable fragment (scFv), a Bi-specific T-cell engager (BiTE), a bispecific killer cell engager (BiKE), a CrossMab, a tri-specific binding partner, or a chimeric antigen receptor (CAR).

In some embodiments, the binding partner of the one or more proteins is conjugated to a detectable label, or a chemotherapeutic agent, a radioisotope, or a toxin.

In some embodiments, the binding partner of the one or more proteins is a component of a fusion protein.

In some embodiments, the binding partner of the one or more proteins comprises a chimeric antigen receptor (CAR).

In some embodiments, the binding partner of the one or more proteins is expressed by a T cell or a natural killer cell.

In some embodiments, the KRAS mutant cancer comprises a mutation selected from a KRAS G12 mutation, a KRAS G13 mutation, a KRAS H61 mutation, and a KRAS K117 mutation.

In some embodiments, the KRAS mutant cancer comprises a KRAS G12 mutation selected from G12C, G12V, G12D, G12S, and G12R mutation.

In some embodiments, the KRAS mutant cancer comprises the KRAS G12C mutation.

In some embodiments, the KRAS mutant cancer comprises the KRAS G13D mutation.

In some embodiments, the KRAS mutant cancer comprises a KRAS H61 mutation selected from Q61H, Q61L, and Q61R mutation.

In some embodiments, the KRAS mutant cancer comprises the KRAS K117N mutation.

In some embodiments, the KRAS mutant cancer also has a mutation in STK11 (LKB1) gene and/or KEAP1 gene.

In some embodiments, the KRAS mutant cancer is a lung cancer, colorectal cancer, or pancreatic cancer.

In some embodiments, the KRAS mutant lung cancer is a KRAS mutant non-small cell lung cancer.

In some embodiments, the KRAS mutant cancer is resistant to a treatment with the SHP2 inhibitor when the SHP2 inhibitor is administered in the absence of the inhibitor of expression or function or degrader of the one or more proteins.

In some embodiments, the subject is human.

In some embodiments, the subject is a veterinary animal.

In some embodiments, the SHP2 inhibitor is selected from BBP-398, TNO155, RMC-4630, JAB-3068, RLY-1971, and ERAS-601, pharmaceutically acceptable salts thereof, and any combinations thereof.

In one aspect, provided herein is a pharmaceutical composition comprising (i) a SHP2 inhibitor, (ii) an inhibitor of expression or function or a degrader of one or more proteins selected from VRK1, RIOK2, ELP4, and ELP5, and (iii) a pharmaceutically acceptable carrier and/or excipient.

In another aspect, provided herein is a pharmaceutical composition comprising (i) a SHP2 inhibitor, (ii) a binding partner of one or more proteins selected from TFRC, SLC7A6OS, GNB2L1, GFER, ATP6AP2, SLC25A19, and PEAR1, and (iii) a pharmaceutically acceptable carrier and/or excipient.

In some embodiments, the SHP2 inhibitor is a selected from BBP-398, TNO155, RMC-4630, JAB-3068, RLY-1971, and ERAS-601.

In one aspect, provided herein is a kit comprising (i) a SHP2 inhibitor, (ii) an inhibitor of expression or function or a degrader of one or more proteins selected from VRK1, RIOK2, ELP4, and ELP5, and (iii) optionally, instructions for use.

In another aspect, provided herein is a kit comprising (i) a SHP2 inhibitor, (ii) a binding partner of one or more proteins selected from TFRC, SLC7A6OS, GNB2L1, GFER, ATP6AP2, SLC25A19, and PEAR1, and (iii) optionally, instructions for use.

In some embodiments, the SHP2 inhibitor is a selected from BBP-398, TNO155, RMC-4630, JAB-3068, RLY-1971, and ERAS-601.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show the results of genome-wide CRISPR/Cas9 screens to identify MRTX-849 synthetic lethal (SL) genes. FIG. 1A shows a schematic representation of a representative CRISPR/Cas9 screening strategy. FIG. 1B shows volcano plots showing results of genome-wide CRISPR/Cas9 screens of KRAS^{G12C} STK11 co-mutated non-small cell lung cancer (NSCLC) cell lines, analyzed by MaGeCK; orange circles indicate select SL genes (False Discovery Rate [FDR]<0.1). FIG. 1C is a Circos plot showing overlap of synthetic lethal (SL) genes in NSCLC lines (FDR<0.1). Outside arcs show SL genes within each line. Inside arcs show SL genes shared in multiple lines (dark orange) and those unique to individual lines (light orange). Purple lines show which cells share a given SL gene. The greater the number of purple links and the size of the dark orange arcs, the greater the overlap of SL genes between cell lines. FIG. 1D shows a heat map showing select SL genes across the four cell lines. Color coding indicates the FDR for each gene in each line (scale at left). FIG. 1E shows bubble plot indicating enriched pathways (p<0.05) of SL genes (FDR<0.1). Datasets used for the pathway analysis are color-coded as shown on the right side. The size of each circle indicates the significance of each pathway assignment.

FIGS. 2A-2G show validation of YAP/TAZ/TEAD pathway genes. FIG. 2A shows trypan blue-based proliferation assays (5 days) on the cell lines H2030, H2122, and H23 treated with TEAD1 or WWTR1 siRNA, as indicated, and/or MRTX-849 (at IC₅₀), normalized to untreated (Control) cells, ****p<0.0001, ***p<0.001, 1-way ANOVA with Tukey's multiple comparisons test. FIG. 2B shows proliferation assays on H2122 cells stably transduced with lentiviruses expressing either of two doxycycline-inducible TEAD1 shRNAs or control shRNA and treated with MRTX-849 (at IC₅₀) of vehicle with or without prior doxycycline (Dox) treatment for 96 hr, ****p<0.0001, 1-way ANOVA with Tukey's multiple comparisons test. FIG. 2C shows proliferation assays on 72 hr Dox-induced H2030 and H2122 cell lines transduced with doxycycline-inducible dominant negative TEAD and treated with MRTX-849 (at IC₅₀) or vehicle, as indicated, ****p<0.0001, 1-way ANOVA with Tukey's multiple comparisons test. FIGS. 2D-2G show MRTX-849 dose-response curves (using modified MTS assay) for the indicated mouse cell lines stably overexpressing TEAD1 (FIG. 2D), WWTR1 (FIG. 2E), YAP1 (FIG. 2F), or YAP1 mutants (FIG. 2G). IC₅₀s were determined by GraphPad Prism.

FIGS. 3A-3I show MRTX-849 treatment induces RHO/ROCK-dependent nuclear translocation of YAP. FIG. 3A shows H2030 cells with co-transfected with the TEAD-responsive 8×GIITC-Luc reporter, normalized to a co-transfected Renilla luciferase construct, and treated with MRTX-849 (at IC₅₀) for 48 hrs, at which time reporter activity (luciferase/Renilla luciferase), was determined. *p<0.05, Student's t-test. FIG. 3B is a heat map showing results of bulk RNA-seq of H2030 and H2122 cells treated with MRTX-849 (at IC₅₀) for 48 hr in triplicate. FIG. 3C is a bubble plot that indicates pathways enriched (p<0.05) in up-regulated genes (FDR<0.1). Datasets (color-coded) used for pathway analysis are indicated at right with the sizes of the circles indicating statistical significance. FIG. 3D shows immunofluorescence images showing YAP1 and DAPI staining of representative fields of H2030 cells treated with MRTX-849 (at IC₅₀) for the indicated times. FIG. 3E shows YAP1 and DAPI immunofluorescence of H2122, H23, and MiaPaca2 cells treated with MRTX-849 (at their respective IC₅₀s) for 48 hrs. FIG. 3F shows MRTX-849 treatment causes increased RHOA activity. H2030 cells were treated with MRTX-849 (at IC₅₀) for 48h, and RHOA-GTP was quantified by ELISA. Luminescence at A490 nm in treated samples normalized to DMSO-treated values is shown. *p<0.05, Student's t-test. FIGS. 3G-3H show that ROCK inhibitor treatment impairs MRTX-849-induced YAP1 nuclear localization. The indicated NSCLC lines were treated with MRTX-849 (at IC₅₀) with or without the ROCK inhibitor Y-27632 (10 μM), and YAP1 localization was assessed by immunofluorescence (with DAPI staining to identify nuclei). FIG. 3I shows trypan blue-based proliferation assays on H2030 and H2122 cell lines treated with MRTX-849 (at IC50) alone or with Y-27632 (10 μM) as indicated, normalized to untreated (Control) cells. ****p<0.0001, ***p<0.001, 1-way ANOVA with Tukey's multiple comparisons test. All immunofluorescence images are representative of three independent experiments. Scale bar=20 μm.

FIGS. 4A-4G show whole-genome CRISPR screens for MRTX-849+TNO155 synthetic lethal genes. FIG. 4A shows the results of genome-wide CRISPR/Cas9 SL screens of H2122, H23, and H2030 cells in the presence or absence of MRTX-849+TNO155 (at doses described in Results) were analyzed using MaGeCK. Select SL genes (FDR<0.1) are indicated by orange circles. FIG. 4B is a bubble plot showing pathways (p<0.05) enriched in SL genes (FDR<0.1). Datasets used for analysis are color-coded at right; the sizes of the circles indicate significance level. FIG. 4C is a Circos plot illustrating the overlap of SL genes (FDR<0.1) between lines. FIG. 4D shows trypan-blue-based proliferation assays on H2030 and H2122 cell lines transfected with TEAD1 siRNA (where indicated) or scrambled control siRNA and treated with vehicle or MRTX-849 and/or TNO155 (at half the IC25 for both drugs), as indicated. ****p<0.0001, 1-way ANOVA and Tukey's multiple comparisons test. FIG. 4E shows proliferation of H2122 cells expressing two different TEAD1 shRNAs and treated with MRTX-849 and/or TNO155, as indicated (IC25 dosage for both of the drugs). ****p<0.0001, 1-way ANOVA and Tukey's multiple comparisons test. FIG. 4F shows effects of dominant negative TEAD and MRTX-849 and/or TNO155 (IC25 dose) on proliferation of H2030 and H2122 cells. ****p<0.0001, 1-way ANOVA and Tukey's multiple comparisons test. FIG. 4G shows representative YAP1 and DAPI immunofluorescence images (from 3 independent experiments) of H2030 cells treated with MRTX-849+TNO155 (each at respective IC₅₀) for 48 hrs.

FIGS. 5A-5F show TEAD inhibition enhances efficacy of MRTX-849 in KRAS^{G12C}-mutant cancers. FIG. 5A shows trypan blue-based proliferation assays (6 days) on H2122, H2030, HCC-44, and H23 lines treated with MYF-03-176 (1 μM) and MRTX-849 (at IC₅₀ for each line) alone or in combination. ****p<0.0001, 1-way ANOVA and Tukey's multiple comparisons test. FIGS. 5B-5D show proliferation assays on the indicated cell lines using VT104 (1 μM) and MRTX-849 (at IC₅₀ for each line) alone or in combination, ****p<0.0001, 1-way ANOVA and Tukey's multiple comparisons test, #synergy by Bliss independent analysis. FIGS. 5E-5F show relative change in tumor volumes after withdrawal of treatments at Day 30, *p<0.0001, 2-way ANOVA.

FIGS. 6A-6G show G12Ci- and G12Ci/SHP2 inhibitor-resistant GEMM and patient samples induce pathways overlapping with SL genes. FIG. 6A shows representative MRI images of KCL mice showing successive development of MTRX-849 and MRTX-849/SHP099 resistance. FIG. 6B shows select enriched pathways (p<0.05) for genes upregulated in MRTX-849-resistant KCL tumors (FDR<0.1) (top) and GSEA demonstrating increased expression of YAP-TAZ signature genes in these tumors (bottom). FIG. 6C shows a snapshot of RPPA showing increased YAP/TAZ levels in MRTX-849-resistant nodules. FIG. 6D shows select enriched pathways (p<0.05) for genes upregulated in MRTX-849/SHP099-resistant KCL tumors (FDR<0.1). FIG. 6E shows snapshot of RPPA showing increased YAP/TAZ levels in MRTX-849/SHP099-resistant nodules. FIGS. 6F-6G show pathway analysis on sc-RNAseq of cells from fresh tumor biopsies of patients with G12Ci (AMG510)- or G12C/SHP2i (MRTX-849+TNO155)-resistant NSCLC, as indicated.

FIGS. 7A-7I show validation of selected additional targets from screens. FIGS. 7A-7B show trypan blue-based proliferation assays (5-days) on H2030, H2122, and H23 cells transfected with RIOK2, VRK1, or scrambled siRNAs (control) and/or treated with MRTX-849 (at IC₅₀), as indicated. ****p<0.0001, ***p<0.001, **p<0.01, 1-way ANOVA and Tukey's multiple comparisons test. FIG. 7C shows trypan blue-based proliferation assays (7 days) on H2030 and H2122 cells treated with VRK-IN-1 (10 μM) and/or MRTX-849 (at IC₅₀), as indicated. ****p<0.0001, ***p<0.001, **p<0.01, 1-way ANOVA and Tukey's multiple comparisons test. FIGS. 7D-7E are the same as FIGS. 7A-7B but with ELP3, ELP5, or scrambled siRNAs, as indicated. FIG. 7F shows trypan blue-based proliferation assays on H2030 and H2122 cells transfected with scrambled or ELP5 siRNAs and treated with MRTX-849 and/or TNO155 (at IC25 of each drug in each line) as indicated. ****p<0.0001, ***p<0.001, **p<0.01, 1-way ANOVA and Tukey's multiple comparisons test. FIGS. 7G-7I show trypan blue-based proliferation assays on H2122 or H2030 cells stably transduced with doxycycline-inducible ELP3, RIOK2, ELP5, or control shRNA or control shRNA, exposed to Dox for 96 h, and treated with MRTX-849 (at IC₅₀ for each line), as indicated, ****p<0.0001, 1-way ANOVA and Tukey's multiple comparisons test.

FIGS. 8A-8C show CRISPR screens for MRTX-849 synthetic lethal genes in NSCLC lines. FIG. 8A shows mutational status of each NSCLC cell line used in MRTX-849 synthetic lethal screens. FIG. 8B shows MRTX-849 IC₅₀ in each line. FIG. 8C shows heat maps (by Pearson coefficient) showing correlation between replicates of each CRISPR/Cas9 screen.

FIGS. 9A-9G show immunoblot confirmation of YAP/TEAD/TAZ pathway si/shRNAs and over-expression constructs. FIGS. 9A-9B show immunoblots showing levels of the indicated proteins after transfection of cognate siRNA (FIG. 9A) or induction of the indicated shRNA (FIG. 9B); GAPDH levels are shown as controls for loading. FIGS. 9C-9G show immunoblots showing levels of the indicated proteins after stable or doxycycline-inducible expression of the indicated genes, as indicated. GAPDH or b-actin levels serve as loading controls.

FIGS. 10A-10E show RHO/ROCK activity regulates MRTX-849 sensitivity in NSCLC lines. FIG. 10A shows qRT-PCR analysis of CYR61 mRNA levels in H2030 and H2122 cell lines treated with MRTX-849 (at IC₅₀) or vehicle for 24 or 48 hrs. FIG. 10B shows representative YAP1 and DAPI immunofluorescence images (from one of three independent experiments) after 24 hr treatment of H2030 cells with MRTX-849 (at IC₅₀) with or without Y-27362 (10 μM). Scale bar=20 μm. FIGS. 10C-10D show quantification of YAP nuclear localization in presence of ROCK-I, in data from FIGS. 3G-3H. *p<0.05, 1-way ANOVA with Tukey's multiple comparisons test. FIG. 10E shows a scheme depicting crosstalk between signaling pathways of KRAS/ERK and RHOA/ROCK/YAP.

FIGS. 11A-11D show results of CRISPR/Cas9 screens of MRTX-849/TN0155 combination. FIG. 11A shows schematic of CRISPR/Cas9 screen strategy. FIG. 11B shows TNO155 IC₅₀ in H2030, H2122, and H23 cells, calculated using GraphPad Prism. FIG. 11C shows heat maps (by Pearson coefficient) showing correlation between replicates of each CRISPR/Cas9 screen. FIG. 11D shows heat map showing shared dropout (SL) genes in MRTX-849/TNO155 (Combo) CRISPR/Cas9 screens. SL genes in both the Combo and at least two MRTX-849 screens were indicated in red; bespoke genes are in black.

FIGS. 12A-12C show effects of pharmacological inhibitors of TEAD. FIG. 12A shows MYF-03-176 dose-response curve (via MTS-based assay) in KCL cells transduced with YAP1 or control expression vector. FIG. 12B shows VT104 (pan-TEAD inhibitor) and VT106 (inactive analog of VT104) dose-response curves (MTS-based assay) in KCL lines overexpressing YAP1 or transduced with control expression vector. FIG. 12C shows trypan blue-based proliferation assays on H2030, H2122, and H23 cells treated with VT104 (1 μM), MRTX-849, and/or TNO155 (at IC25 of each drug) alone or in combination, ****p<0.0001, 1-way ANOVA and Tukey's multiple comparisons test.

FIGS. 13A-13B show RNA-seq analysis of G12C therapy-resistant GEMM and patient samples. FIGS. 13A-13B show heat maps of top 100 genes from bulk RNA-seq analysis of MRTX-849-resistant (FIG. 13A) and MRTX-84/SHP099-resistant (FIG. 13B) KCL nodules.

FIGS. 14A-14F show confirmation of SL gene depletion by si/shRNAs. FIGS. 14A-14C show immunoblots showing cognate protein levels in H2122, H2030, and H23 cells 72 h post-transfection with RIOK2, ELP3, or ELP5 siRNAs, as indicated. FIGS. 14D-14F show immunoblots showing cognate protein levels in H2122 and H2030 cells (as indicated) expressing inducible RIOK2, ELP3, ELP5 or control shRNAs after Dox treatment for 72h.

FIGS. 15A-15D illustrate that synthetic lethal (SL) genes and resistance genes disclosed herein belong to specific signaling pathways. Shown are genes whose knockout causes G12Ci resistance (red) or synthetic lethality (blue) in the Rat Sarcoma-Mitogen-Activated Protein Kinase (RAS-MAPK) (FIG. 15A), Hippo (FIG. 15B), Mammalian Target of Rapamycin (mTOR) (FIG. 15C), or tRNA synthesis and modification (FIG. 15D) signaling pathways. All genes shown “scored” in at least 2 G12C-mutant non-small cell lung cancer (NSCLC) lines; those that scored in 3 or 4 lines are indicated by the corresponding number.

### DETAILED DESCRIPTION

To more globally define the landscape of potential combination strategies, the present application describes, among other things, the use of genome-wide CRISPR/Cas9 screening to identify genes whose inactivation is synthetic lethal with MRTX-849 alone or in combination with TNO-155 in KRAS^{G12C}-mutant NSCLC cell lines. Owing to the association of these genotypes with poor therapeutic response, lines were chosen with co-mutation of STK11; three also were KEAP1-defective. Results disclosed herein identify potentially druggable targets for use in combination with KRAS inhibitors including, without limitation, KRAS G12C inhibitors. Additionally, results disclosed herein identify potentially druggable targets for use in combination with SHP2 inhibitors.

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The terms “inhibit” or “inhibition” as used herein refer to reducing a function or activity to an extent sufficient to achieve a desired biological or physiological effect. Inhibition may be complete or partial.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

It must also be noted that, as used in the specification and the appended claims, the singular forms “a,” “an,” and “the” include plural references, unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents that operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

### Methods of the Invention

### Methods of the Invention Comprising KRAS Inhibitors

In one aspect, the present disclosure provides a method for overcoming or preventing resistance of a Kirsten Rat Sarcoma Viral Oncogene Homolog (KRAS) mutant cancer cell to growth inhibition and/or cell death induction by a KRAS inhibitor. In certain aspects, the method for overcoming or preventing resistance of the KRAS mutant cancer cell to growth inhibition and/or cell death induction by a KRAS inhibitor comprises inhibiting in the KRAS mutant cancer cell expression or function of one or more proteins such as, but not limited to, e.g., Vaccinia-Related Kinase 1 (VRK1), RIO Kinase 2 (RIOK2), Exostosin Glycosyltransferase 1 (EXT1), Exostosin Glycosyltransferase 2 (EXT2), Elongator Acetyltransferase Complex Subunit 2 (ELP2), Elongator Acetyltransferase Complex Subunit 3 (ELP3), Elongator Acetyltransferase Complex Subunit 5 (ELP5), Protein Kinase N2 (PKN2), Phosphogluconate Dehydrogenase (PGD), Phosphoglucomutase 2 (PGM2), Rho Associated Coiled-Coil Containing Protein Kinase 1 (ROCK1), Rho Associated Coiled-Coil Containing Protein Kinase 2 (ROCK2), Adaptor Related Protein Complex 2 Subunit Sigma 1 (AP2S1), Erb-B2 Receptor Tyrosine Kinase 3 (ERBB3), Growth Factor Receptor Bound Protein 2 (GRB2), CRK Proto-Oncogene, Adaptor Protein (CRK), SRC Proto-Oncogene, Non-Receptor Tyrosine Kinase (SRC), P21 (RAC1) Activated Kinase 2 (PAK2), N-Deacetylase and N-Sulfotransferase 1 (NDST1), SHOC2 Leucine Rich Repeat Scaffold Protein (SHOC2), importin 11 (IPO11), Yes-associated protein (YAP), WW domain-containing transcription regulator 1 (WWTR1, also known as Transcriptional co-Activator with PDZ-binding motif [TAZ]), TEA domain family member (TEAD), Mechanistic Target of Rapamycin Kinase (MTOR), Regulatory Associated Protein of MTOR Complex 1 (RPTOR), Transcription Factor IIIC (TFIIIC), General Transcription Factor IIIC Subunit 1 (GTF3C1), TATA-Box Binding Protein (TBP), Hydroxysteroid 17-Beta Dehydrogenase 10 (HSD17B10), Processing of Precursor 5, Ribonuclease P/MRP Subunit (POP5), Ribonuclease P/MRP Subunit P21 (RPP21), RNA 2′,3′-Cyclic Phosphate and 5′-OH Ligase (RTCB), TRNA Splicing Endonuclease Subunit 2 (TSEN2), Ubiquitin Related Modifier 1 (URM1), Elongator Acetyltransferase Complex Subunit 4 (ELP4), ELP1 (also known as IKB Kinase Complex Associated Protein [IKBKAP]), Adenosine Deaminase TRNA Specific 3 (ADAT3), Molybdenum Cofactor Synthesis 3 (MOCS3), KTI12 Chromatin Associated Homolog (KTI12), Isoleucyl-TRNA Synthetase (IARS), Tyrosyl-TRNA Synthetase (YARS), Sep (O-Phosphoserine) TRNA:Sec (Selenocysteine) TRNA Synthase (SEPSECS), Prolyl-TRNA Synthetase 2 (PARS2), Tyrosyl-TRNA Synthetase 2 (YARS2), Aspartyl-TRNA Synthetase 2 (DARS2), and Leucyl-TRNA Synthetase 2 (LARS2). In some embodiments, the expression or function of all and each of proteins that are members of the elongated complex, e.g., Elongator Acetyltransferase Complex 1-6 (ELP1-6), may be inhibited. In some embodiments, the expression or function of any of elongated complex proteins ELP1, ELP2, ELP3, ELP4, ELP5, or ELP6, or a combination thereof, may be inhibited.

In another aspect, the present disclosure provides a method for enhancing sensitivity of a KRAS mutant cancer cell to a KRAS inhibitor. In certain aspects, the method for enhancing sensitivity of the KRAS mutant cancer cell to a KRAS inhibitor comprises inhibiting in the KRAS mutant cancer cell expression or function of one or more proteins such as, but not limited to, e.g., VRK1, RIOK2, EXT1, EXT2, ELP2, ELP3, ELP5, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and/or LARS2. In some embodiments, the expression or function of all and each of proteins that are members of the elongated complex (ELP1-6) may be inhibited. In some embodiments, the expression or function of any of elongator complex proteins ELP1, ELP2, ELP3, ELP4, ELP5, or ELP6, or a combination thereof, may be inhibited.

Without wishing to be bound by theory, the KRAS gene encodes KRAS protein, a p21 GTPase of the small GTPase superfamily. KRAS cycles between an active guanosine triphosphate (GTP)-bound state and an inactive guanosine diphosphate (GDP)-bound state. Regarding cell signaling, KRAS protein functions as a molecular switch that can transmit extracellular signals of receptor tyrosine kinases (e.g., EGFR) thereby initiating a signal transduction cascade. Active, GTP-bound KRAS can interact with numerous effectors, stimulating multiple signaling pathways (e.g., PI3K-AKT-MTOR, RAF-MEK-ERK), which can affect various cellular processes (e.g., proliferation, cellular survival, cytoskeletal organization).

Two predominant KRAS protein isoforms can arise from alternative RNA splicing, KRAS4A and KRAS4B. KRAS4B is the predominant splice variant and is expressed in many tissues, contributing to its frequent study in cancer research. Additionally, there is substantial KRAS4A expression in certain tissues (e.g., intestine, heart, stomach, and kidney) and cancers (e.g., colon cancer). KRAS is an oncogene and is one of the most frequently mutated across a broad range of cancers. A single nucleotide substitution or a single amino acid substitution may be accountable for an activating mutation in KRAS. KRAS protein with abnormal activity may direct cells to proliferate abnormally (e.g., uncontrollably). KRAS gene mutations have been linked to various types of cancer such as, but not limited to, cancers described herein.

In some embodiments, a KRAS protein (which may also be referred to as, e.g., KRAS protein, KRAS, or GTPase KRAS) described herein can include, without limitation, any of various recombinant or naturally-occurring forms of KRAS, or variants or homologs thereof, that are capable of KRAS activity (e.g., within at least 50%, 60%, 70%, 75%, 80%, 85%, 90%, 95%, 96%, 97%, 98%, 99% or 100% activity compared to a naturally-occurring KRAS protein). In some aspects, the variants or homologs have at least 80%, 85%, 90%, 95%, 96%, 97%, 98%, 99% or 100% amino acid sequence identity across either the whole sequence or a portion of the sequence (e.g., a 50, 75, 100, 125, 150, 175, 200, or larger continuous amino acid portion) compared to a naturally-occurring KRAS protein. In some embodiments, the KRAS protein can be either isoform 2A or isoform 2B.

In some embodiments, an inhibitor of the present disclosure can negatively affect (e.g., decrease) the expression or function (e.g., activity) of one or more of the various proteins described herein. For example, an inhibitor described herein may decrease the expression or function (e.g., activity) of KRAS, VRK1, RIOK2, EXT1, EXT2, ELP1, ELP2, ELP3, ELP4, ELP5, ELP6, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and/or LARS2) relative to the expression or function (e.g., activity) of such protein in the absence of the inhibitor. In some aspects, an inhibition described herein may refer to reduction of a disease or reduction of symptoms of disease (e.g., cancer). Thus, inhibition can include, fully or in part, partially or totally decreasing, preventing, or blocking stimulation; partially or totally decreasing, preventing, or delaying activation; or inactivating, desensitizing, decreasing, or down-regulating signal transduction, enzymatic activity, or an amount of a protein (e.g., KRAS, VRK1, RIOK2, EXT1, EXT2, ELP1, ELP2, ELP3, ELP4, ELP5, ELP6, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and/or LARS2). Similarly, an inhibitor can be a protein, a nucleic acid, or generally a compound that inhibits a receptor or a protein (e.g., by delaying, binding, decreasing, partially or totally blocking, partially or totally preventing, partially or totally inactivating, desensitizing, or down-regulating activity (e.g., KRAS protein activity, VRK1 protein activity, RIOK2 protein activity, EXT1 protein activity, EXT2 protein activity, ELP1 protein activity, ELP2 protein activity, ELP3 protein activity, ELP4 protein activity, ELP5 protein activity, ELP6 protein activity, PKN2 protein activity, PGD protein activity, PGM2 protein activity, ROCK1 protein activity, ROCK2 protein activity, AP2S1 protein activity, ERBB3 protein activity, GRB2 protein activity, CRK protein activity, SRC protein activity, PAK2 protein activity, NDST1 protein activity, SHOC2 protein activity, IPO11 protein activity, YAP protein activity, WWTR1 protein activity, TEAD protein activity, MTOR protein activity, RPTOR protein activity, TFIIIC protein activity, GTF3C1 protein activity, TBP protein activity, HSD17B10 protein activity, POP5 protein activity, RPP21 protein activity, RTCB protein activity, TSEN2 protein activity, URM1 protein activity, ADAT3 protein activity, MOCS3 protein activity, KTI12 protein activity, IARS protein activity, YARS protein activity, SEPSECS protein activity, PARS2 protein activity, YARS2 protein activity, DARS2 protein activity, and/or LARS2 protein activity).

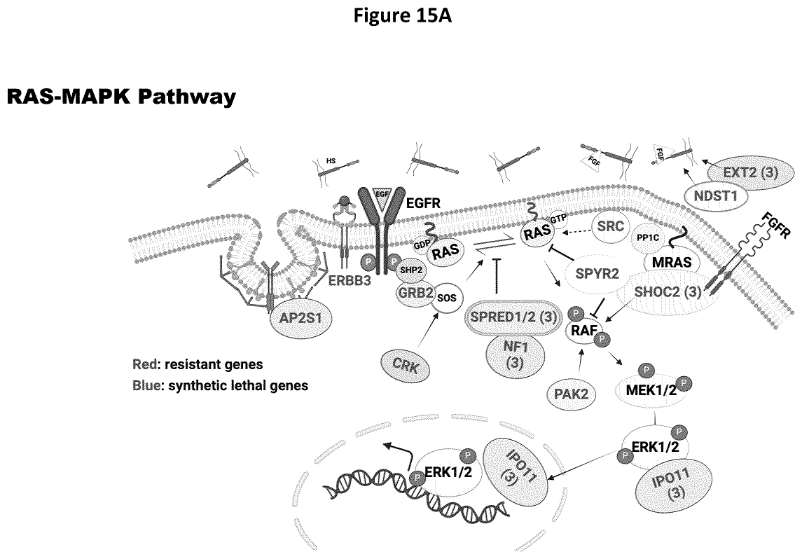

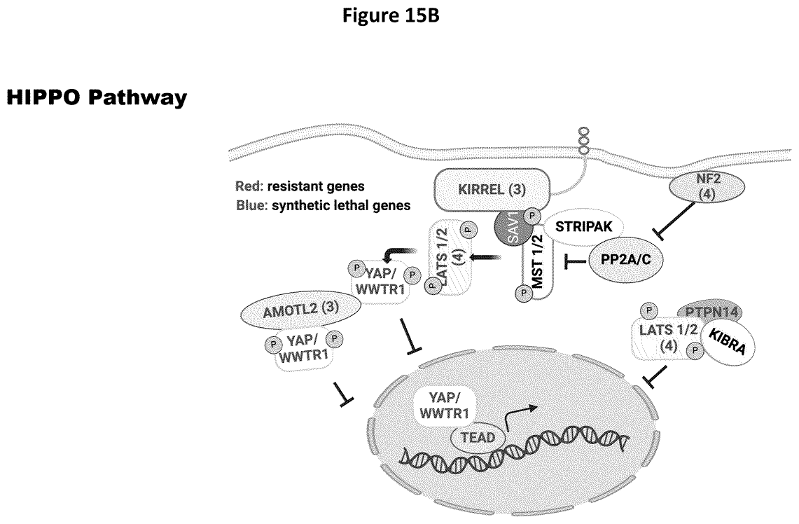

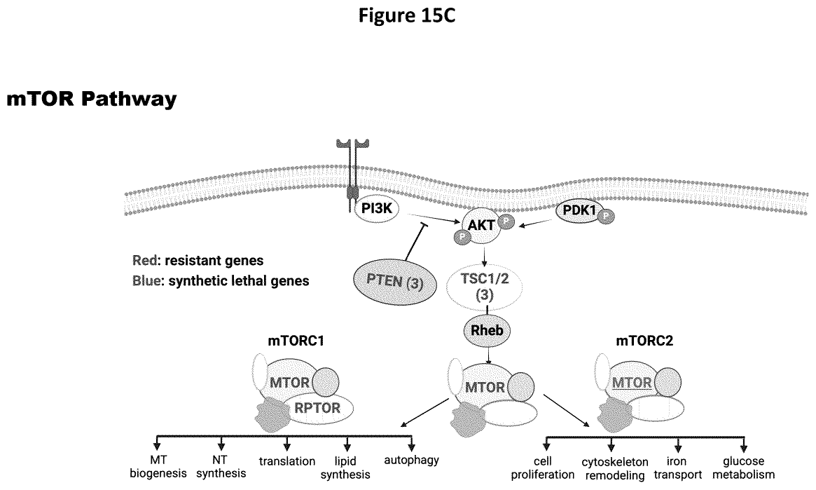

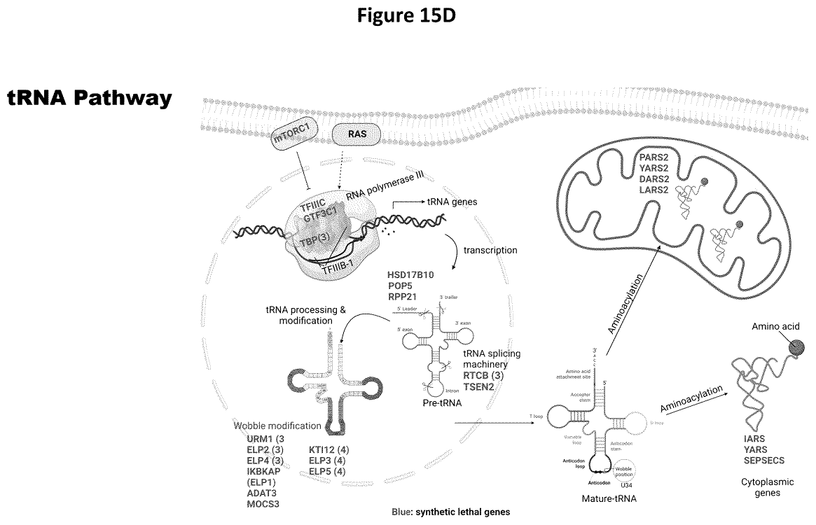

In some embodiments of methods for overcoming or preventing resistance of a KRAS mutant cancer cell to growth inhibition and/or cell death induction by a KRAS inhibitor or for enhancing sensitivity of a KRAS mutant cancer cell to a KRAS inhibitor, the method comprises inhibiting in a KRAS mutant cancer cell expression or function of one or more proteins associated with a Rat Sarcoma-Mitogen-Activated Protein Kinase (RAS-MAPK) signaling pathway (see, e.g., FIG. 15A), a Hippo signaling pathway (see, e.g., FIG. 15B), an Mammalian Target of Rapamycin (mTOR) signaling pathway (see, e.g., FIG. 15C), and/or a tRNA synthesis and modification signaling pathway (see, e.g., FIG. 15D).

In some embodiments of methods for overcoming or preventing resistance of a KRAS mutant cancer cell to growth inhibition and/or cell death induction by a KRAS inhibitor or for enhancing sensitivity of a KRAS mutant cancer cell to a KRAS inhibitor, the method comprises inhibiting in a KRAS mutant cancer cell expression or function of one or more proteins selected from, e.g., TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and/or LARS2.

In some embodiments of methods for overcoming or preventing resistance of a KRAS mutant cancer cell to growth inhibition and/or cell death induction by a KRAS inhibitor or for enhancing sensitivity of a KRAS mutant cancer cell to a KRAS inhibitor, the method comprises inhibiting in a KRAS mutant cancer cell expression or function of one or more proteins selected from, e.g., AP2S1, ERBB3, GRB2, CRK, SRC, PAK2, EXT2, NDST1, SHOC2, and/or IPO11.

In some embodiments of methods for overcoming or preventing resistance of a KRAS mutant cancer cell to growth inhibition and/or cell death induction by a KRAS inhibitor or for enhancing sensitivity of a KRAS mutant cancer cell to a KRAS inhibitor, the method comprises inhibiting in a KRAS mutant cancer cell expression or function of, e.g., MTOR protein and/or RPTOR protein.

In some embodiments of methods for overcoming or preventing resistance of a KRAS mutant cancer cell to growth inhibition and/or cell death induction by a KRAS inhibitor or for enhancing sensitivity of a KRAS mutant cancer cell to a KRAS inhibitor, the method comprises inhibiting in a KRAS mutant cancer cell expression or function of one or more proteins selected from, e.g., VRK1, ELP2, ELP3, PKN2, RIOK2, EXT1, and/or EXT2. In some embodiments, the method comprises inhibiting in the KRAS mutant cancer cell expression or function of VRK1 protein, PKN2 protein, and/or RIOK2 protein.

In various embodiments of any of the above-described methods, the expression or function of a KRAS protein described herein may be inhibited in a KRAS mutant cancer cell described herein by a KRAS inhibitor described herein. As a non-limiting example, KRAS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, KRAS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of VRK1 protein. As a non-limiting example, VRK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, VRK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RIOK2 protein. As a non-limiting example, RIOK2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RIOK2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EXT1 protein. As a non-limiting example, EXT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EXT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EXT2 protein. As a non-limiting example, EXT2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EXT2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP1 protein. As a non-limiting example, ELP1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP2 protein. As a non-limiting example, ELP2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP3 protein. As a non-limiting example, ELP3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP4 protein. As a non-limiting example, ELP4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP5 protein. As a non-limiting example, ELP5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP6 protein. As a non-limiting example, ELP6 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP6 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PKN2 protein. As a non-limiting example, PKN2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PKN2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PGD protein. As a non-limiting example, PGD protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PGD protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PGM2 protein. As a non-limiting example, PGM2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PGM2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ROCK1 protein. As a non-limiting example, ROCK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ROCK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ROCK2 protein. As a non-limiting example, ROCK2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ROCK2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, a method of the present disclosure (e.g., a method for overcoming or preventing resistance of a KRAS mutant cancer cell to growth inhibition and/or cell death induction by a KRAS inhibitor or a method for enhancing sensitivity of a KRAS mutant cancer cell to a KRAS inhibitor) comprises inhibiting in a KRAS mutant cancer cell the kinase activity of VRK1 protein and/or RIOK2 protein and/or PKN2 protein.

In various embodiments, the kinase activity of VRK1 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the kinase activity of VRK1 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the kinase activity of RIOK2 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the kinase activity of RIOK2 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the kinase activity of PKN2 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the kinase activity of PKN2 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of AP2S1 protein. As a non-limiting example, AP2S1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, AP2S1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ERBB3 protein. As a non-limiting example, ERBB3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ERBB3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GRB2 protein. As a non-limiting example, GRB2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GRB2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CRK protein. As a non-limiting example, CRK protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CRK protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SRC protein. As a non-limiting example, SRC protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SRC protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PAK2 protein. As a non-limiting example, PAK2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PAK2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NDST1 protein. As a non-limiting example, NDST1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NDST1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SHOC2 protein. As a non-limiting example, SHOC2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SHOC2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IPO11 protein. As a non-limiting example, IPO11 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IPO11 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of YAP protein. As a non-limiting example, YAP protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, YAP protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of WWTR1 protein. As a non-limiting example, WWTR1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, WWTR1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TEAD protein. As a non-limiting example, TEAD protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TEAD protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MTOR protein. As a non-limiting example, MTOR protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MTOR protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPTOR protein. As a non-limiting example, RPTOR protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPTOR protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TFIIIC protein. As a non-limiting example, TFIIIC protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TFIIIC protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GTF3C1 protein. As a non-limiting example, GTF3C1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GTF3C1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TBP protein. As a non-limiting example, TBP protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TBP protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of HSD17B10 protein. As a non-limiting example, HSD17B10 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, HSD17B10 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POP5 protein. As a non-limiting example, POP5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POP5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPP21 protein. As a non-limiting example, RPP21 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPP21 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RTCB protein. As a non-limiting example, RTCB protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RTCB protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSEN2 protein. As a non-limiting example, TSEN2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSEN2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of URM1 protein. As a non-limiting example, URM1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, URM1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ADAT3 protein. As a non-limiting example, ADAT3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ADAT3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MOCS3 protein. As a non-limiting example, MOCS3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MOCS3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of KTI12 protein. As a non-limiting example, KTI12 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, KTI12 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IARS protein. As a non-limiting example, IARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of YARS protein. As a non-limiting example, YARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, YARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SEPSECS protein. As a non-limiting example, SEPSECS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SEPSECS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PARS2 protein. As a non-limiting example, PARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of YARS2 protein. As a non-limiting example, YARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, YARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DARS2 protein. As a non-limiting example, DARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of LARS2 protein. As a non-limiting example, LARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, a method of the present disclosure (e.g., a method for overcoming or preventing resistance of a KRAS mutant cancer cell to growth inhibition and/or cell death induction by a KRAS inhibitor or a method for enhancing sensitivity of a KRAS mutant cancer cell to a KRAS inhibitor) comprises inhibiting in a KRAS mutant cancer cell the ATPase activity of RIOK2 protein.

In various embodiments, the ATPase activity of RIOK2 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the ATPase activity of RIOK2 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments of any of the above-described methods, the method comprises administering to a KRAS mutant cancer cell an inhibitor of expression or function of the one or more proteins described herein (e.g., VRK1, RIOK2, EXT1, EXT2, ELP1, ELP2, ELP3, ELP4, ELP5, ELP6, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2) or a degrader of the one or more proteins described herein (e.g., VRK1, RIOK2, EXT1, EXT2, ELP1, ELP2, ELP3, ELP4, ELP5, ELP6, PKN2, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2).

In some embodiments, the inhibitor of expression or function of the one or more proteins or the degrader of the one or more proteins described herein is administered to the KRAS mutant cancer cell simultaneously or sequentially with the KRAS inhibitor. As a non-limiting example, the inhibitor of expression or function of the one or more proteins or the degrader of the one or more proteins described herein can be administered to the KRAS mutant cancer cell simultaneously with the KRAS inhibitor in one composition. As another non-limiting example, the inhibitor of expression or function of the one or more proteins or the degrader of the one or more proteins described herein can be administered to the KRAS mutant cancer cell simultaneously with the KRAS inhibitor in different compositions. As yet another non-limiting example, the inhibitor of expression or function of the one or more proteins or the degrader of the one or more proteins described herein can be administered to the KRAS mutant cancer cell sequentially with the KRAS inhibitor in different compositions.

In some embodiments, when the inhibitor of expression or function of the one or more proteins or the degrader of the one or more proteins described herein and the KRAS inhibitor are administered to the KRAS mutant cancer cell sequentially (e.g., in different compositions), the inhibitor of expression or function of the one or more proteins or the degrader of the one or more proteins may be administered as a first component of a dosing regimen and the KRAS inhibitor may be administered as a second component of a dosing regimen (i.e., the inhibitor of expression or function of the one or more proteins or the degrader of the one or more proteins may be administered before the KRAS inhibitor).

In some embodiments, when the inhibitor of expression or function of the one or more proteins or the degrader of the one or more proteins described herein and the KRAS inhibitor are administered to the KRAS mutant cancer cell sequentially (e.g., in different compositions), the KRAS inhibitor may be administered as a first component of a dosing regimen and the inhibitor of expression or function of the one or more proteins or the degrader of the one or more proteins may be administered as a second component of a dosing regimen (i.e., the KRAS inhibitor may be administered before the inhibitor of expression or function of the one or more proteins or the degrader of the one or more proteins).

In some embodiments, when the one or more proteins is ROCK1 protein and/or ROCK2 protein, an inhibitor of expression or function or degrader of ROCK1 protein and/or ROCK2 protein can be, e.g., AR-12286, Fasudil, Ripasudil, Netarsudil, KD025 (Belumosudil), AT13148, GSK269962, H1152, GSK429286, pharmaceutically acceptable salts thereof, and any combinations thereof.

In some embodiments, when the one or more proteins is MTOR protein and/or RPTOR protein, an inhibitor of expression or function or degrader of MTOR protein and/or RPTOR protein can be, e.g., Rapamycin, RAD001, TORIN, pharmaceutically acceptable salts thereof, and any combinations thereof.

In some embodiments, when the one or more proteins is SRC protein, an inhibitor of expression or function or degrader of SRC protein can be, e.g., Dasatanib, Bosutinib, Sarcatanib, pharmaceutically acceptable salts thereof, and any combinations thereof.

In some embodiments, a KRAS polynucleotide (e.g., DNA or RNA) comprises one or more mutations. In certain embodiments, the KRAS polynucleotide comprises one or more mutations in exon 1 at codons 12 or 13. In some embodiments, the KRAS polynucleotide comprises one or multiple mutations at codons 18, 61, 63, 117, 119, or 146. In some embodiments, the KRAS polynucleotide comprises one or more mutations at positions that correspond to amino acid residues 12, 13, 18, 19, 20, 22, 24, 26, 36, 59, 61, 63, 64, 68, 110, 116, 117, 119, 146, 147, 158, 164, 176, or a combination thereof. In certain embodiments, KRAS polynucleotide comprises one or multiple mutations at positions that correspond to amino acid residues G12V, G12D, G12C, G12A, G12S, G12F, G12R, G12L, G12T, G13C, G13D, G13V, G13R, G13H, G13A, Q61K, Q61H, Q61L, Q61R, Q61P, Q61E, E62K, E63K, R68S, R68G, R68M, D69G, V14G, V14I, S17G, A18D, A146T, A146P, A146V, A146G, L19F, T20M, T2OR, I21R, Q22K, I24N, I24V, T35A, I36L, I36M, T50I, D57N, T58I, N26K, H27N, D33E, P34L, P34R, A59G, A59S, A59T, A59E, G60V, Y71D, Y71C, Y64D, Y64N, Y64H, Y96C, Y96D, M72V, M72T, T74P, D92Y, P110S, N116H, N116S, K117N, K117E, K117R, K147N, C118S, Q131H, R135T, R161G, R164Q, T144I, D153V, A155D, F156L, T158A, K176Q, or a combination thereof.

In some aspects, a KRAS mutation can comprise a mutation in one or multiple codons in the KRAS gene. In certain aspects, the KRAS mutation refers to a mutation at, for example, without limitation, codon 12, at codon 13, at codon 61, or at codon 117, or a combination thereof.

In some embodiments, a KRAS mutation can comprise a mutation in one or multiple amino acids in the KRAS protein. Examples of amino acid mutations comprise, but are not limited to, amino acid substitutions, deletions, and/or insertions. Amino acid substitution means that an amino acid residue is substituted (i.e., replaced) for a different amino acid residue at the same position. Amino acid deletion means that an amino acid residue is deleted (i.e., removed). Amino acid residues that are inserted may be inserted at any position and may be added such that some or all of the inserted amino acid residues are immediately adjacent to one another or may be added such that none of the inserted amino acid residues are immediately adjacent to one another. In some aspects, a KRAS mutation refers to, e.g., a G12 mutation, a G13 mutation, a H61 mutation, or a K117 mutation, or a combination thereof. In certain aspects, the KRAS mutation refers to a G12C mutation, a G12V mutation, a G12D mutation, a G13D mutation, a Q61H mutation, a Q61L mutation, or a Q61R mutation, or a combination thereof.

In some embodiments, a KRAS mutant cancer cell described herein may comprise any number of various mutations in KRAS described herein, or combinations thereof. For instance, without limitation, a KRAS mutant cancer cell may comprise a mutation such as, but not limited to, a KRAS G12 mutation, a KRAS G13 mutation, a KRAS H61 mutation, or a KRAS K117 mutation.

In some embodiments, when the KRAS mutant cancer cell described herein comprises a KRAS G12 mutation, the KRAS G12 mutation may be, e.g., a G12C mutation, a G12V mutation, a G12D mutation, a G12S mutation, or a G12R mutation.

In some embodiments, a KRAS mutant cancer cell described herein comprises a KRAS G12C mutation. In some embodiments, e.g., when the KRAS mutant cell comprises a KRAS G12C mutation, the KRAS inhibitor can be a KRAS G12C inhibitor (G12Ci). In some embodiments, the KRAS G12C inhibitor (G12Ci) is adagrasib (MRTX-849). In some embodiments, the KRAS G12C inhibitor (G12Ci) is sotorasib (AMG510).

In various embodiments, the KRAS mutant cancer cell comprises a KRAS G13D mutation. In various embodiments, the KRAS mutant cancer cell comprises a KRAS H61 mutation. Non-limiting examples of a KRAS H61 mutation include a Q61H mutation, a Q61L mutation, and a Q61R mutation.

In some embodiments, the KRAS mutant cancer cell described herein comprises a KRAS K117N mutation.

In some embodiments, a KRAS mutant cancer cell comprising any of the above-described mutations in KRAS may, in addition, also comprise a mutation, e.g., in STK11 (also called Liver kinase B1 [LKB1]) gene and/or Kelch Like ECH Associated Protein 1 (KEAP1) gene.

In some embodiments, a KRAS mutant cancer cell of the present disclosure may comprise deletion or reduced expression of one or more genes associated with and/or predictive of resistance of the KRAS mutant cancer cell to treatment with the KRAS inhibitor. In some embodiments, the one or more genes associated with and/or predictive of resistance of the KRAS mutant cancer cell to treatment with the KRAS inhibitor may be associated with a RAS-MAPK signaling pathway (see, e.g., FIG. 15A), a Hippo signaling pathway (see, e.g., FIG. 15B), an mTOR signaling pathway (see, e.g., FIG. 15C), and/or a tRNA synthesis and modification signaling pathway (see, e.g., FIG. 15D). Non-limiting examples of genes associated with and/or predictive or resistance of the KRAS mutant cancer cell to treatment with the KRAS inhibitor include, e.g., Sprouty-related, EVH1 domain-containing protein 1 (SPRED1), SPRED2, Neurofibromin 1 (NF1), Synaptophysin-related gene 2 (SPYR2), Angiomotin-like protein 2 (AMOTL2), Large tumor suppressor kinase 1 (LATS1), LATS2, Kin of IRRE-like protein (KIRREL, also known as Nephrin-like protein 1), Neurofibromin 2 (NF2, also known as Merlin), Tyrosine-protein phosphatase non-receptor type 14 (PTPN14), Phosphatase and tensin homolog (PTEN), Tuberous sclerosis complex 1 (TSC1), and TSC2.

In various embodiments of any of the above-described methods, the method comprises deletion or reduced expression in the KRAS mutant cancer cell of SPRED1 gene. As a non-limiting example, SPRED1 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SPRED1 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, SPRED1 gene can be deleted in a KRAS mutant cell disclosed herein.

In various embodiments of any of the above-described methods, the method comprises deletion or reduced expression in the KRAS mutant cancer cell of SPRED2 gene. As a non-limiting example, SPRED2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SPRED2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, SPRED2 gene can be deleted in a KRAS mutant cell disclosed herein.

In various embodiments of any of the above-described methods, the method comprises deletion or reduced expression in the KRAS mutant cancer cell of NF1 gene. As a non-limiting example, NF1 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NF1 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, NF1 gene can be deleted in a KRAS mutant cell disclosed herein.

In various embodiments of any of the above-described methods, the method comprises deletion or reduced expression in the KRAS mutant cancer cell of SPYR2 gene. As a non-limiting example, SPYR2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SPYR2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, SPYR2 gene can be deleted in a KRAS mutant cell disclosed herein.

In various embodiments of any of the above-described methods, the method comprises deletion or reduced expression in the KRAS mutant cancer cell of AMOTL2 gene. As a non-limiting example, AMOTL2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, AMOTL2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, AMOTL2 gene can be deleted in a KRAS mutant cell disclosed herein.

In various embodiments of any of the above-described methods, the method comprises deletion or reduced expression in the KRAS mutant cancer cell of LATS1 gene. As a non-limiting example, LATS1 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LATS1 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, LATS1 gene can be deleted in a KRAS mutant cell disclosed herein.

In various embodiments of any of the above-described methods, the method comprises deletion or reduced expression in the KRAS mutant cancer cell of LATS2 gene. As a non-limiting example, LATS2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LATS2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, LATS2 gene can be deleted in a KRAS mutant cell disclosed herein.

In various embodiments of any of the above-described methods, the method comprises deletion or reduced expression in the KRAS mutant cancer cell of KIRREL gene. As a non-limiting example, KIRREL gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, KIRREL gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, KIRREL gene can be deleted in a KRAS mutant cell disclosed herein.

In various embodiments of any of the above-described methods, the method comprises deletion or reduced expression in the KRAS mutant cancer cell of NF2 gene. As a non-limiting example, NF2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NF2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, NF2 gene can be deleted in a KRAS mutant cell disclosed herein.

In various embodiments of any of the above-described methods, the method comprises deletion or reduced expression in the KRAS mutant cancer cell of PTPN14 gene. As a non-limiting example, PTPN14 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PTPN14 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, PTPN14 gene can be deleted in a KRAS mutant cell disclosed herein.

In various embodiments of any of the above-described methods, the method comprises deletion or reduced expression in the KRAS mutant cancer cell of PTEN gene. As a non-limiting example, PTEN gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PTEN gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, PTEN gene can be deleted in a KRAS mutant cell disclosed herein.

In various embodiments of any of the above-described methods, the method comprises deletion or reduced expression in the KRAS mutant cancer cell of TSC1 gene. As a non-limiting example, TSC1 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSC1 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, TSC1 gene can be deleted in a KRAS mutant cell disclosed herein.

In various embodiments of any of the above-described methods, the method comprises deletion or reduced expression in the KRAS mutant cancer cell of TSC2 gene. As a non-limiting example, TSC2 gene expression may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSC2 gene expression may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, TSC2 gene can be deleted in a KRAS mutant cell disclosed herein.

KRAS mutant cancer cells of the present disclosure can be any KRAS mutant cell type known to those of skill in the art. As an example, without limitation, KRAS mutant cancer(s) cells can be liver cells (e.g., hepatocytes), stomach cells (e.g., parietal cells, endocrine cells, chief cells, mucous cells), intestinal cells (e.g., epithelial cells) including colon cells and cells of the rectum, kidney cells (e.g., endothelial cells, interstitial cells, immune cells), cardiac cells (e.g., myocardial contractile cells, myocardial conducting cells), brain cells (e.g., glia, neurons), lung cells (e.g., epithelial cells), ovarian cells, breast cells, prostate cells (e.g., basal, neuroendocrine, luminal), bladder cells (e.g., urothelial cells), blood cells (e.g., red blood cells, white blood cells, platelets), pancreatic cells (e.g., cells that line the duct of the pancreas, exocrine cells, neuroendocrine cells), or cells of the lymph system (e.g., B lymphocytes, T lymphocytes, natural killer cells). In some embodiments, the KRAS mutant cancer cell is in a subject (e.g., a human, a veterinary animal, or an experimental model).

A KRAS mutant cancer cell described herein may be derived from a KRAS mutant cancer. A KRAS mutant cancer can be, e.g., without limitation, any cancer of the present disclosure. In certain embodiments, the KRAS mutant cancer is lung cancer, colorectal cancer, or pancreatic cancer. In some embodiments, the lung cancer is non-small cell lung cancer. Additional non-limiting examples of cancers are discussed below.

Non-limiting examples of KRAS inhibitors which may be used in the practice of the present disclosure include adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some embodiments, the KRAS inhibitor is the KRAS G12C inhibitor (G12Ci) adagrasib (MRTX-849). In some embodiments, the KRAS inhibitor is the KRAS G12C inhibitor (G12Ci) sotorasib (AMG510).

In some embodiments, a KRAS mutant cancer cell may be in a subject. In some embodiments, the subject is human. In some embodiments, the subject is a veterinary animal (e.g., cats, dogs, cows, horses, sheep, pigs, etc.) or an experimental animal model.

### Therapeutic Methods Comprising KRAS Inhibitors

Any of the various methods or compositions (e.g., pharmaceutical compositions) described herein may be used in the practice of various therapeutic applications (in vivo and ex vivo) and as research tools.

In one aspect, the present disclosure provides a method of treating a KRAS mutant cancer in a subject in need thereof. In certain aspects, the method of treating a KRAS mutant cancer in a subject in need thereof comprises administering to the subject an effective amount of a KRAS inhibitor and an inhibitor of the expression or function or a degrader of one or more proteins such as, but not limited to, e.g., VRK1, RIOK2, PKN2, EXT1, EXT2, ELP2, ELP3, ELP5, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2. In some embodiments, the method of treating a KRAS mutant cancer in a subject in need thereof comprises administering to the subject an effective amount of a KRAS inhibitor and an inhibitor of expression or function or a degrader of all and each of proteins which are members of the elongator complex, e.g., Elongator Acetyltransferase Complex 1-6 (ELP1-6), including ELP1, ELP2, ELP3, ELP4, ELP5, or ELP6, or a combination thereof.

In various embodiments of the above-described method, the method comprises administering to the subject an effective amount of a KRAS inhibitor and an inhibitor of the expression or function or a degrader of one or more proteins selected from, e.g., TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and LARS2.

In various embodiments of the above-described method, the method comprises administering to the subject an effective amount of a KRAS inhibitor and an inhibitor of the expression or function or a degrader of one or more proteins selected from, e.g., AP2S1, ERBB3, GRB2, CRK, SRC, PAK2, EXT2, NDST1, SHOC2, and IPO11.

In various embodiments of the above-described method, the method comprises administering to the subject an effective amount of a KRAS inhibitor and an inhibitor of the expression or function or a degrader of MTOR protein and/or RPTOR protein.

In various embodiments of the above-described method, the method comprises administering to the subject an effective amount of a KRAS inhibitor and an inhibitor of the expression or function or a degrader of one or more proteins selected from, e.g., VRK1, PKN2, ELP2, ELP3, RIOK2, EXT1, and EXT2. In some embodiments, the method comprises administering to the subject an effective amount of a KRAS inhibitor and an inhibitor of the expression or function or a degrader of VRK1 protein, PKN2 protein, and/or RIOK2 protein.

In various embodiments of any of the above-described methods, the expression or function of a KRAS protein described herein may be inhibited in a subject in need thereof by administering to the subject an effective amount of a KRAS inhibitor described herein. As a non-limiting example, the KRAS inhibitor may inhibit KRAS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the KRAS inhibitor may inhibit KRAS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of VRK1 protein may be administered to a subject in need thereof. As a non-limiting example, the inhibitor of the expression or function of VRK1 protein may inhibit VRK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of VRK1 protein may inhibit VRK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of VRK1 protein may degrade VRK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of VRK1 protein may degrade VRK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of RIOK2 protein may be administered to a subject in need thereof. As a non-limiting example, the inhibitor of expression or function of RIOK2 protein may inhibit RIOK2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of expression or function of RIOK2 protein may inhibit RIOK2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RIOK2 protein may degrade RIOK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RIOK2 protein may degrade RIOK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of PKN2 protein may be administered to a subject in need thereof. As a non-limiting example, the inhibitor of the expression or function of PKN2 protein may inhibit PKN2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PKN2 protein may inhibit PKN2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PKN2 protein may degrade PKN2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PKN2 protein may degrade PKN2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of EXT 1 protein may be administered to a subject in need thereof. As a non-limiting example, the inhibitor of expression or function of EXT1 protein may inhibit EXT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of expression or function of EXT1 protein may inhibit EXT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EXT1 protein may degrade EXT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EXT1 protein may degrade EXT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of EXT2 protein may be administered to a subject in need thereof. As a non-limiting example, the inhibitor of the expression or function of EXT2 protein may inhibit EXT2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EXT2 protein may inhibit EXT2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EXT2 protein may degrade EXT2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EXT2 protein may degrade EXT2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP1 protein may be administered to a subject in need thereof. As a non-limiting example, the inhibitor of expression or function of ELP1 protein may inhibit ELP1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of expression or function of ELP1 protein may inhibit ELP1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP1 protein may degrade ELP1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP1 protein may degrade ELP1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP2 protein may be administered to a subject in need thereof. As a non-limiting example, the inhibitor of the expression or function of ELP2 protein may inhibit ELP2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP2 protein may inhibit ELP2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP2 protein may degrade ELP2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP2 protein may degrade ELP2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP3 protein may be administered to a subject in need thereof. As a non-limiting example, the inhibitor of the expression or function of ELP3 protein may inhibit ELP3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP3 protein may inhibit ELP3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP3 protein may degrade ELP3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP3 protein may degrade ELP3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP4 protein may be administered to a subject in need thereof. As a non-limiting example, the inhibitor of the expression or function of ELP4 protein may inhibit ELP4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP4 protein may inhibit ELP4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP4 protein may degrade ELP4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP4 protein may degrade ELP4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP5 protein may be administered to a subject in need thereof. As a non-limiting example, the inhibitor of the expression or function of ELP5 protein may inhibit ELP5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP5 protein may inhibit ELP5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP5 protein may degrade ELP5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP5 protein may degrade ELP5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ELP6 protein may be administered to a subject in need thereof. As a non-limiting example, the inhibitor of the expression or function of ELP6 protein may inhibit ELP6 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor of the expression or function of ELP6 protein may inhibit ELP6 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP6 protein may degrade ELP6 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP6 protein may degrade ELP6 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of PGD protein may be administered to a subject in need thereof. As a non-limiting example, the inhibitor of the expression or function of PGD protein may inhibit PGD protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PGD protein may inhibit PGD protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PGD protein may degrade PGD protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PGD protein may degrade PGD protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of PGM2 protein may be administered to a subject in need thereof. As a non-limiting example, the inhibitor of the expression or function of PGM2 protein may inhibit PGM2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PGM2 protein may inhibit PGM2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PGM2 protein may degrade PGM2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PGM2 protein may degrade PGM2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ROCK1 protein may be administered to a subject in need thereof. As a non-limiting example, the inhibitor of the expression or function of ROCK1 protein may inhibit ROCK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ROCK1 protein may inhibit ROCK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ROCK1 protein may degrade ROCK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ROCK1 protein may degrade ROCK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of the expression or function and/or a degrader of ROCK2 protein may be administered to a subject in need thereof. As a non-limiting example, the inhibitor of the expression or function of ROCK2 protein may inhibit ROCK2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ROCK2 protein may inhibit ROCK2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ROCK2 protein may degrade ROCK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ROCK2 protein may degrade ROCK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of AP2S1 protein may be administered to a subject in need thereof. As a non-limiting example, the inhibitor of the expression or function of AP2S1 protein may inhibit AP2S1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of AP2S1 protein may inhibit AP2S1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of AP2S1 protein may degrade AP2S1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of AP2S1 protein may degrade AP2S1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of ERBB3 protein may be administered to a subject in need thereof. As a non-limiting example, the inhibitor of the expression or function of ERBB3 protein may inhibit ERBB3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ERBB3 protein may inhibit ERBB3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ERBB3 protein may degrade ERBB3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ERBB3 protein may degrade ERBB3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of GRB2 protein may be administered to a subject in need thereof. As a non-limiting example, the inhibitor of the expression or function of GRB2 protein may inhibit GRB2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GRB2 protein may inhibit GRB2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GRB2 protein may degrade GRB2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GRB2 protein may degrade GRB2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of CRK protein may be administered to a subject in need thereof. As a non-limiting example, the inhibitor of the expression or function of CRK protein may inhibit CRK protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CRK protein may inhibit CRK protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CRK protein may degrade CRK protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CRK protein may degrade CRK protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of SRC protein may be administered to a subject in need thereof. As a non-limiting example, the inhibitor of the expression or function of SRC protein may inhibit SRC protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SRC protein may inhibit SRC protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SRC protein may degrade SRC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SRC protein may degrade SRC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of PAK2 protein may be administered to a subject in need thereof. As a non-limiting example, the inhibitor of the expression or function of PAK2 protein may inhibit PAK2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PAK2 protein may inhibit PAK2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PAK2 protein may degrade PAK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PAK2 protein may degrade PAK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of NDST1 protein may be administered to a subject in need thereof. As a non-limiting example, the inhibitor of the expression or function of NDST1 protein may inhibit NDST1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NDST1 protein may inhibit NDST1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NDST1 protein may degrade NDST1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NDST1 protein may degrade NDST1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of SHOC2 protein may be administered to a subject in need thereof. As a non-limiting example, the inhibitor of the expression or function of SHOC2 protein may inhibit SHOC2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SHOC2 protein may inhibit SHOC2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SHOC2 protein may degrade SHOC2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SHOC2 protein may degrade SHOC2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of IPO11 protein may be administered to a subject in need thereof. As a non-limiting example, the inhibitor of the expression or function of IPO11 protein may inhibit IPO11 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IPO11 protein may inhibit IPO11 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IPO11 protein may degrade IPO11 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IPO11 protein may degrade IPO11 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of YAP protein may be administered to a subject in need thereof. As a non-limiting example, the inhibitor of the expression or function of YAP protein may inhibit YAP protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of YAP protein may inhibit YAP protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of YAP protein may degrade YAP protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of YAP protein may degrade YAP protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of WWTR1 protein may be administered to a subject in need thereof. As a non-limiting example, the inhibitor of the expression or function of WWTR1 protein may inhibit WWTR1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of WWTR1 protein may inhibit WWTR1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of WWTR1 protein may degrade WWTR1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of WWTR1 protein may degrade WWTR1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of TEAD protein may be administered to a subject in need thereof. As a non-limiting example, the inhibitor of the expression or function of TEAD protein may inhibit TEAD protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TEAD protein may inhibit TEAD protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TEAD protein may degrade TEAD protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TEAD protein may degrade TEAD protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of MTOR protein may be administered to a subject in need thereof. As a non-limiting example, the inhibitor of the expression or function of MTOR protein may inhibit MTOR protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MTOR protein may inhibit MTOR protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MTOR protein may degrade MTOR protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MTOR protein may degrade MTOR protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of RPTOR protein may be administered to a subject in need thereof. As a non-limiting example, the inhibitor of the expression or function of RPTOR protein may inhibit RPTOR protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPTOR protein may inhibit RPTOR protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPTOR protein may degrade RPTOR protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPTOR protein may degrade RPTOR protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of TFIIIC protein may be administered to a subject in need thereof. As a non-limiting example, the inhibitor of the expression or function of TFIIIC protein may inhibit TFIIIC protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TFIIIC protein may inhibit TFIIIC protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TFIIIC protein may degrade TFIIIC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TFIIIC protein may degrade TFIIIC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of GTF3C1 protein may be administered to a subject in need thereof. As a non-limiting example, the inhibitor of the expression or function of GTF3C1 protein may inhibit GTF3C1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GTF3C1 protein may inhibit GTF3C1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GTF3C1 protein may degrade GTF3C1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GTF3C1 protein may degrade GTF3C1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of TBP protein may be administered to a subject in need thereof. As a non-limiting example, the inhibitor of the expression or function of TBP protein may inhibit TBP protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TBP protein may inhibit TBP protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TBP protein may degrade TBP protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TBP protein may degrade TBP protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of HSD17B10 protein may be administered to a subject in need thereof. As a non-limiting example, the inhibitor of the expression or function of HSD17B10 protein may inhibit HSD17B10 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of HSD17B10 protein may inhibit HSD17B10 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of HSD17B10 protein may degrade HSD17B10 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of HSD17B10 protein may degrade HSD17B10 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of POP5 protein may be administered to a subject in need thereof. As a non-limiting example, the inhibitor of the expression or function of POP5 protein may inhibit POP5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POP5 protein may inhibit POP5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POP5 protein may degrade POP5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POP5 protein may degrade POP5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of RPP21 protein may be administered to a subject in need thereof. As a non-limiting example, the inhibitor of the expression or function of RPP21 protein may inhibit RPP21 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPP21 protein may inhibit RPP21 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPP21 protein may degrade RPP21 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPP21 protein may degrade RPP21 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of RTCB protein may be administered to a subject in need thereof. As a non-limiting example, the inhibitor of the expression or function of RTCB protein may inhibit RTCB protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RTCB protein may inhibit RTCB protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RTCB protein may degrade RTCB protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RTCB protein may degrade RTCB protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of TSEN2 protein may be administered to a subject in need thereof. As a non-limiting example, the inhibitor of the expression or function of TSEN2 protein may inhibit TSEN2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSEN2 protein may inhibit TSEN2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSEN2 protein may degrade TSEN2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSEN2 protein may degrade TSEN2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of URM1 protein may be administered to a subject in need thereof. As a non-limiting example, the inhibitor of the expression or function of URM1 protein may inhibit URM1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of URM1 protein may inhibit URM1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of URM1 protein may degrade URM1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of URM1 protein may degrade URM1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of ADAT3 protein may be administered to a subject in need thereof. As a non-limiting example, the inhibitor of the expression or function of ADAT3 protein may inhibit ADAT3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ADAT3 protein may inhibit ADAT3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ADAT3 protein may degrade ADAT3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ADAT3 protein may degrade ADAT3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of MOCS3 protein may be administered to a subject in need thereof. As a non-limiting example, the inhibitor of the expression or function of MOCS3 protein may inhibit MOCS3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MOCS3 protein may inhibit MOCS3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MOCS3 protein may degrade MOCS3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MOCS3 protein may degrade MOCS3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of KTI12 protein may be administered to a subject in need thereof. As a non-limiting example, the inhibitor of the expression or function of KTI12 protein may inhibit KTI12 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of KTI12 protein may inhibit KTI12 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of KTI12 protein may degrade KTI12 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of KTI12 protein may degrade KTI12 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of IARS protein may be administered to a subject in need thereof. As a non-limiting example, the inhibitor of the expression or function of IARS protein may inhibit IARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IARS protein may inhibit IARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IARS protein may degrade IARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IARS protein may degrade IARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of YARS protein may be administered to a subject in need thereof. As a non-limiting example, the inhibitor of the expression or function of YARS protein may inhibit YARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of YARS protein may inhibit YARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of YARS protein may degrade YARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of YARS protein may degrade YARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of SEPSECS protein may be administered to a subject in need thereof. As a non-limiting example, the inhibitor of the expression or function of SEPSECS protein may inhibit SEPSECS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SEPSECS protein may inhibit SEPSECS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SEPSECS protein may degrade SEPSECS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SEPSECS protein may degrade SEPSECS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of PARS2 protein may be administered to a subject in need thereof. As a non-limiting example, the inhibitor of the expression or function of PARS2 protein may inhibit PARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PARS2 protein may inhibit PARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PARS2 protein may degrade PARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PARS2 protein may degrade PARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of YARS2 protein may be administered to a subject in need thereof. As a non-limiting example, the inhibitor of the expression or function of YARS2 protein may inhibit YARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of YARS2 protein may inhibit YARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of YARS2 protein may degrade YARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of YARS2 protein may degrade YARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of DARS2 protein may be administered to a subject in need thereof. As a non-limiting example, the inhibitor of the expression or function of DARS2 protein may inhibit DARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DARS2 protein may inhibit DARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DARS2 protein may degrade DARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DARS2 protein may degrade DARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of a KRAS inhibitor described herein and an effective amount of an inhibitor of expression or function and/or a degrader of LARS2 protein may be administered to a subject in need thereof. As a non-limiting example, the inhibitor of the expression or function of LARS2 protein may inhibit LARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of LARS2 protein may inhibit LARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of LARS2 protein may degrade LARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of LARS2 protein may degrade LARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the method comprises administering to the subject an inhibitor of the kinase activity of VRK1 protein, PKN2 protein, and/or RIOK2 protein.

In various embodiments, the inhibitor of kinase activity may inhibit the kinase activity of VRK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of kinase activity may inhibit the kinase activity of VRK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of kinase activity may inhibit the kinase activity of PKN2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of kinase activity may inhibit the kinase activity of PKN2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of kinase activity may inhibit the kinase activity of RIOK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of kinase activity may inhibit the kinase activity of RIOK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the method comprises administering to the subject an inhibitor of the ATPase activity of RIOK2 protein.

In various embodiments, the inhibitor of ATPase activity may inhibit the ATPase activity of RIOK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of ATPase activity may inhibit the ATPase activity of RIOK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the KRAS inhibitor and the inhibitor of the expression or function or degrader of the one or more proteins described herein are administered to the subject simultaneously. In some embodiments, the KRAS inhibitor and the inhibitor of the expression or function or degrader of the one or more proteins described herein are administered to the subject simultaneously in one composition. In some embodiments, the KRAS inhibitor and the inhibitor of the expression or function or degrader of the one or more proteins described herein are administered to the subject simultaneously in different compositions. In various embodiments, the KRAS inhibitor and the inhibitor of the expression or function or degrader of the one or more proteins described herein are administered to the subject sequentially.

In some embodiments, when the inhibitor of the expression or function of the one or more proteins or the degrader of the one or more proteins described herein and the KRAS inhibitor are administered to the subject sequentially (e.g., in different compositions), the inhibitor of the expression or function of the one or more proteins or the degrader of the one or more proteins may be administered as a first component of a dosing regimen and the KRAS inhibitor may be administered as a second component of a dosing regimen (i.e., the inhibitor of the expression or function of the one or more proteins or the degrader of the one or more proteins may be administered before the KRAS inhibitor).

In some embodiments, when the inhibitor of the expression or function of the one or more proteins or the degrader of the one or more proteins described herein and the KRAS inhibitor are administered to the subject sequentially (e.g., in different compositions), the KRAS inhibitor may be administered as a first component of a dosing regimen and the inhibitor of the expression or function of the one or more proteins or the degrader of the one or more proteins may be administered as a second component of a dosing regimen (i.e., the KRAS inhibitor may be administered before the inhibitor of the expression or function of the one or more proteins or the degrader of the one or more proteins).

In some embodiments, the KRAS inhibitor and/or the inhibitor of the expression or function or degrader of the one or more proteins described herein can be administered to the subject by way of any route of administration of the present disclosure. As a non-limiting example, the KRAS inhibitor and/or the inhibitor of the expression or function or degrader of the one or more proteins described herein can be administered orally or intravenously.

In some embodiments, when the one or more proteins is ROCK1 protein and/or ROCK2 protein, an inhibitor of the expression or function or degrader of ROCK1 protein and/or ROCK2 protein can be, e.g., AR-12286, Fasudil, Ripasudil, Netarsudil, KD025 (Belumosudil), AT13148, GSK269962, H1152, GSK429286, pharmaceutically acceptable salts thereof, and any combinations thereof.

In some embodiments, when the one or more proteins is MTOR protein and/or RPTOR protein, an inhibitor of the expression or function or degrader of MTOR protein and/or RPTOR protein can be, e.g., Rapamycin, RAD001, TORIN, pharmaceutically acceptable salts thereof, and any combinations thereof.

In some embodiments, when the one or more proteins is SRC protein, an inhibitor of the expression or function or degrader of SRC protein can be, e.g., Dasatanib, Bosutinib, Sarcatanib, pharmaceutically acceptable salts thereof, and any combinations thereof.

In some embodiments, a KRAS mutant cancer cell described herein may comprise any number of various mutations in KRAS described herein, or combinations thereof. For instance, without limitation, a KRAS mutant cancer cell may comprise a mutation such as, but not limited to, a KRAS G12 mutation, a KRAS G13 mutation, a KRAS H61 mutation, or a KRAS K117 mutation. In some embodiments, when the KRAS mutant cancer cell described herein comprises a KRAS G12 mutation, the KRAS G12 mutation may be, e.g., a G12C mutation, a G12V mutation, a G12D mutation, a G12S mutation, or a G12R mutation.

In some embodiments, the KRAS mutant cancer cell described herein comprises a KRAS G12C mutation. In some embodiments, e.g., when the KRAS mutant cell comprises a KRAS G12C mutation, the KRAS inhibitor can be a KRAS G12C inhibitor (G12Ci). In some embodiments, the KRAS G12C inhibitor (G12Ci) is adagrasib (MRTX-849). In some embodiments, the KRAS G12C inhibitor (G12Ci) is sotorasib (AMG510).

In various embodiments, the KRAS mutant cancer cell described herein comprises a KRAS G13D mutation. In various embodiments, the KRAS mutant cancer cell comprises a KRAS H61 mutation. Non-limiting examples of a KRAS H61 mutation include a Q61H mutation, a Q61L mutation, and a Q61R mutation.

In some embodiments, the KRAS mutant cancer cell described herein comprises a KRAS K117N mutation.

In some embodiments, a KRAS mutant cancer cell comprising any of the above described mutations in KRAS may, in addition, also comprise a mutation, e.g., in STK11 (also called Liver kinase B1 [LKB1]) gene and/or Kelch Like ECH Associated Protein 1 (KEAP1) gene.

In some embodiments, a KRAS mutant cancer cell of the present disclosure may comprise deletion or reduced expression of one or more genes associated with and/or predictive of resistance of the KRAS mutant cancer cell to treatment with the KRAS inhibitor. In some embodiments, the one or more genes associated with and/or predictive of resistance of the KRAS mutant cancer cell to treatment with the KRAS inhibitor may be associated with a RAS-MAPK signaling pathway (see, e.g., FIG. 15A), a Hippo signaling pathway (see, e.g., FIG. 15B), an mTOR signaling pathway (see, e.g., FIG. 15C), and/or a tRNA synthesis and modification signaling pathway (see, e.g., FIG. 15D). Non-limiting examples of genes associated with and/or predictive or resistance of the KRAS mutant cancer cell to treatment with the KRAS inhibitor include, e.g., Sprouty-related, EVH1 domain-containing protein 1 (SPRED1), SPRED2, Neurofibromin 1 (NF1), Synaptophysin-related gene 2 (SPYR2), Angiomotin-like protein 2 (AMOTL2), Large tumor suppressor kinase 1 (LATS1), LATS2, Kin of IRRE-like protein (KIRREL, also known as Nephrin-like protein 1), Neurofibromin 2 (NF2, also known as Merlin), Tyrosine-protein phosphatase non-receptor type 14 (PTPN14), Phosphatase and tensin homolog (PTEN), Tuberous sclerosis complex 1 (TSC1), and TSC2.

As discussed above, KRAS mutant cancer cells can comprise any KRAS mutant cell type known to those of skill in the art such as, but not limited to, any of various KRAS mutant cancer cell described herein.

In some embodiments, a KRAS mutant cancer cell described herein may be derived from a KRAS mutant cancer. In certain embodiments, the KRAS mutant cancer is lung cancer, colorectal cancer, or pancreatic cancer. In some embodiments, the lung cancer is non-small cell lung cancer.

In some embodiments, the cancer is a glioma cancer. In certain embodiments, the cancer is ovarian cancer. In certain embodiments, the cancer is a lung cancer. In some embodiments, the cancer is non-small cell lung cancer. In some embodiments, the cancer is a head and neck cancer. In some embodiments, the cancer is a colorectal cancer. In some embodiments, the cancer is a stomach cancer. In some embodiments, the cancer is a renal cancer. In some embodiments, the cancer is adult renal cell carcinoma or pediatric renal cell carcinoma. In some embodiments, the cancer is a skin cancer. In some embodiments, the cancer is a cervical cancer. In some embodiments, the cancer is brain cancer. In some embodiments, the cancer is breast cancer. In some embodiments, the cancer is triple negative breast cancer. In some embodiments, the cancer is a prostate cancer. In further embodiments, the cancer is a bladder cancer.

In certain embodiments the cancer is a hematologic malignancy (e.g., leukemia, a lymphoma, or a myeloma). Leukemia includes, but is not limited to, acute myeloid leukemia (AML), chronic lymphocytic leukemia (CLL), acute lymphocytic leukemia (ALL), liver acute lymphoblastic leukemia, and chronic myeloid leukemia (CML). Non-limiting examples of lymphoma are non-Hodgkin's lymphoma or Hodgkin's lymphoma. In some embodiments, the lymphoma is anaplastic large cell lymphoma (ALCL). In further embodiments, the non-Hodgkin's lymphoma is Diffuse Large B-cell Lymphoma (DLBCL).

In some embodiments, the cancer is neuroblastoma, inflammatory myofibroblastic tumor, colonic adeno-carcinoma, glioblastoma, glioblastoma multiforme, anaplastic thyroid cancer, cholangiocarcinoma, angiosarcoma, epithelioid hemangioendothelioma, intrahepatic cholangiocarcinoma, thyroid cancer, spitzoid neoplasms, sarcomas, astrocytoma, brain lower grade glioma, secretory breast carcinoma, mammary analogue carcinoma, congenital mesoblastic nephroma, congenital fibrosarcomas, Ph-like acute lymphoblastic leukemia, thyroid carcinoma, head and neck squamous cell carcinoma, pediatric glioma CML, lung squamous carcinoma, ovarian serous cystadenocarcinoma, skin cutaneous melanoma, castrate-resistant prostate cancer, serous and clear cell endometrial cancer, oral cancer, endometrial cancer, endocrine cancer, gastric cancer, esophageal cancer, laryngeal cancer, colon cancer, bone cancer, testicular cancer, rectal cancer, kidney cancer, liver cancer, stomach cancer, metastatic non-small cell lung cancer, colorectal cancer, metastatic colorectal cancer, pancreatic cancer, metastatic pancreatic cancer, metastatic uterine cancer. In some embodiments, the cancer is adenocarcinomas, adenomatoid tumors, alveolar (bronchiolar) carcinoma, ampullary carcinoma, angioma, basal cell carcinoma, benign chondroma, botryoid sarcoma (embryonal rhabdomyosarcoma), bronchial adenoma, bronchogenic carcinoma undifferentiated large cell, bronchogenic carcinoma undifferentiated small cell, bronchogenic carcinoma, carcinoid tumors, carcinomas, cervical carcinoma, chondroblastoma, chondromatous hamartoma, chondromyxofibroma, chondrosarcoma, choriocarcinoma, clear cell carcinoma, congenital tumors, dermatofibroma, ductal adenocarcinoma, dysgerminoma, embryonal carcinoma, endometrial carcinoma, ependymoma, esophageal squamous cell carcinoma, Ewing's sarcoma, fallopian tubes cancer, fibroadenoma, fibromas, gall bladder carcinoma, gastrinoma, germinoma (pinealoma), gliomas, gliomatosis, glucagonoma, granuloma, granulosa-thecal cell tumors, hamartoma, hemangiomas, hepatoblastoma, hepatocellular adenoma, hepatoma (hepatocellular carcinoma), insulinoma, interstitial cell carcinoma, intraepithelial carcinoma, Kaposi's sarcoma, keloids, large bowel cancers, leiomyomas, leiomyosarcomas, lipomas, liposarcoma, malignant fibrous histiocytoma, malignant giant cell tumor chordoma, malignant lymphoma (reticulum cell sarcoma), malignant melanoma, malignant teratoma, medulloblastoma, melanoma, meningio sarcoma, meningioma, mesothelioma, moles dysplastic nevi, mucinous cystadenocarcinoma, multiple myeloma, myelodysplastic syndrome, myeloproliferative diseases, myxoma, neurofibroma, oligodendroglioma, osteitis deformans, osteochronfroma (osteocartilaginous exostoses), osteogenic sarcoma (osteosarcoma), osteoid osteoma and giant cell tumors, osteoma, ovarian carcinoma, pre-tumor cervical dysplasia, prostate sarcoma, retinoblastoma, rhabdomyoma, Rhabdomyosarcoma, schwannoma, seminoma, Sertoli-Leydig cell tumors, small bowel cancers, spinal cord neurofibroma, squamous cell carcinomas, teratocarcinoma, teratomas, testis cancers, transitional cell carcinomas, tubular adenoma, unclassified carcinomas, urethral cancers, vaginal cancers, villous adenoma, vipoma, vulvar cancers, Wilm's tumor (nephroblastoma), and xanthoma.

In some embodiments, the KRAS mutant cancer is uterine cancer or gastric cancer.

In some embodiments, the KRAS mutant cancer described herein may be resistant to a treatment with a KRAS inhibitor (e.g., any of various KRAS inhibitors described herein) when the KRAS inhibitor is administered in the absence of the inhibitor of the expression or function or degrader of the one or more proteins described herein. Non-limiting examples of KRAS inhibitors include adagrasib (MRTX-849), sotorasib (AMG510), divarasib (GDC-6036), MRTX1133, ARS1620, BI-1701963, N—(I-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some embodiments, the KRAS inhibitor is the KRAS G12C inhibitor (G12Ci) adagrasib (MRTX-849). In some embodiments, the KRAS inhibitor is the KRAS G12C inhibitor (G12Ci) sotorasib (AMG510).

In some embodiments, the subject is human. In some embodiments, the subject is a veterinary animal (e.g., cats, dogs, cows, horses, sheep, pigs, etc.) or an experimental animal model.

### Methods of the Invention Comprising SHP2 Inhibitors

In one aspect, the present disclosure provides a method for overcoming or preventing resistance of a KRAS mutant cancer cell to growth inhibition and/or cell death induction by an SHP2 inhibitor. In certain aspects, the method for overcoming or preventing resistance of the KRAS mutant cancer cell to growth inhibition and/or cell death induction by an SHP2 inhibitor comprises inhibiting in the KRAS mutant cancer cell the expression or function of one or more proteins such as, but not limited to, e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, MP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and/or PEAR1.

In another aspect, the present disclosure provides a method for enhancing the sensitivity of a KRAS mutant cancer cell to an SHP2 inhibitor. In certain aspects, the method for enhancing the sensitivity of the KRAS mutant cancer cell to an SHP2 inhibitor comprises inhibiting in the KRAS mutant cancer cell the expression or function of one or more proteins such as, but not limited to, e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and/or PEAR1.

In some embodiments, a KRAS protein (which may also be referred to as, e.g., KRAS protein, KRAS, or GTPase KRAS) described herein can include, without limitation, any of various recombinant or naturally-occurring forms of KRAS, or variants or homologs thereof, that are capable of KRAS activity (e.g., within at least 50%, 60%, 70%, 75%, 80%, 85%, 90%, 95%, 96%, 97%, 98%, 99% or 100% activity compared to a naturally-occurring KRAS protein). In some aspects, the variants or homologs have at least 80%, 85%, 90%, 95%, 96%, 97%, 98%, 99% or 100% amino acid sequence identity across either the whole sequence or a portion of the sequence (e.g., a 50, 75, 100, 125, 150, 175, 200, or larger continuous amino acid portion) compared to a naturally-occurring KRAS protein. In some embodiments, the KRAS protein can be either isoform 2A or isoform 2B.

In some embodiments, an inhibitor of the present disclosure can negatively affect (e.g., decrease) the expression or function (e.g., activity) of one or more of various proteins described herein. For example, an inhibitor described herein may decrease the expression or function (e.g., activity) of SHP2, VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and/or PEAR1 relative to the expression or function (e.g., activity) of such protein in the absence of the inhibitor. In some aspects, an inhibition described herein may refer to reduction of a disease or reduction of symptoms of disease (e.g., cancer). Thus, inhibition can include, fully or in part, partially or totally decreasing, preventing, or blocking stimulation; partially or totally decreasing, preventing, or delaying activation; or inactivating, desensitizing, decreasing, or down-regulating signal transduction by, enzymatic activity of, or the amount of a protein (e.g., SHP2, VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and/or PEAR1). Similarly, an inhibitor can be a protein, a nucleic acid, or generally a compound that inhibits a receptor or a protein (e.g., by delaying, binding, decreasing, partially or totally blocking, partially or totally preventing, partially or totally inactivating, desensitizing, or down-regulating activity (e.g., SHP2 protein activity, VRK1 protein activity, RIOK2 protein activity, ELP4 protein activity, ELP5 protein activity, ENO1 protein activity, GAPDH protein activity, MARS2 protein activity, ATP6V1F protein activity, PRMT5 protein activity, COQ2 protein activity, DBR1 protein activity, DTYMK protein activity, DKC1 protein activity, RNMT protein activity, PPP1R8 protein activity, HSD17B10 protein activity, DOLK protein activity, ALG1 protein activity, UROD protein activity, POLR3H protein activity, PGD protein activity, TSEN2 protein activity, RNASEH2A protein activity, GUK1 protein activity, TSFM protein activity, NELFB protein activity, DOHH protein activity, EXOSC5 protein activity, RPE protein activity, CSTF1 protein activity, RTEL1 protein activity, WARS2 protein activity, UTP23 protein activity, POLG2 protein activity, THG1L protein activity, RARS2 protein activity, RAD51D protein activity, LARS2 protein activity, SDHB protein activity, CPSF4 protein activity, PDPK1 protein activity, DDX10 protein activity, VARS2 protein activity, PDSS2 protein activity, PSMG4 protein activity, DHX33 protein activity, COASY protein activity, VHL protein activity, RNGTT protein activity, PPP1R2 protein activity, NOL11 protein activity, CTDNEP1 protein activity, ISG20L2 protein activity, ERCC2 protein activity, TOP3A protein activity, MTG2 protein activity, BRF1 protein activity, PIK3C3 protein activity, IARS protein activity, AURKAIP1 protein activity, UQCRFS1 protein activity, PRMT1 protein activity, DDX59 protein activity, MARS protein activity, TOE1 protein activity, SARS2 protein activity, CDIPT protein activity, YARS protein activity, CARS2 protein activity, PPP2R4 protein activity, RPP21 protein activity, UGP2 protein activity, DPAGT1 protein activity, PYROXD1 protein activity, MTOR protein activity, HARS2 protein activity, NARS protein activity, TSC1 protein activity, POLR3C protein activity, QRSL1 protein activity, RPIA protein activity, SDHC protein activity, DDX56 protein activity, EIF4E protein activity, DDX46 protein activity, IMPDH2 protein activity, SOD2 protein activity, UBE2M protein activity, GATC protein activity, TSC2 protein activity, PMPCA protein activity, TSEN54 protein activity, FOXM1 protein activity, FARS2 protein activity, CTPS1 protein activity, PARS2 protein activity, ALG2 protein activity, EIF2B3 protein activity, CMPK1 protein activity, DHDDS protein activity, SAE1 protein activity, NARS2 protein activity, PNKP protein activity, PDSS1 protein activity, POLR3K protein activity, AHCY protein activity, NAE1 protein activity, UBIAD1 protein activity, RPUSD4 protein activity, EARS2 protein activity, GMPPB protein activity, LIAS protein activity, PPP4C protein activity, NSUN4 protein activity, DLD protein activity, TRMT5 protein activity, AASDHPPT protein activity, EIF5A protein activity, POT1 protein activity, DHX9 protein activity, LONP1 protein activity, PPP6C protein activity, SKIV2L2 protein activity, PTDSS1 protein activity, USP5 protein activity, VPS52 protein activity, TKT protein activity, TRMT61A protein activity, N6AMT1 protein activity, GGPS1 protein activity, EFTUD1 protein activity, ACAD9 protein activity, SETD1A protein activity, IPO11 protein activity, EIF3I protein activity, METTL16 protein activity, MASTL protein activity, DDX51 protein activity, ADAT3 protein activity, ZNRD1 protein activity, OGT protein activity, IDI1 protein activity, IMP4 protein activity, FTSJ3 protein activity, EXOSC8 protein activity, GSG2 protein activity, PI4KA protein activity, NSMCE2 protein activity, DDX52 protein activity, DDOST protein activity, CSNK2B protein activity, UBA2 protein activity, RABGGTA protein activity, SOD1 protein activity, TRIT1 protein activity, TYMS protein activity, RNF168 protein activity, UBE2I protein activity, GARS protein activity, IPO13 protein activity, SMARCB1 protein activity, EIF2B1 protein activity, RNASEH1 protein activity, MCAT protein activity, XRN2 protein activity, POP5 protein activity, CS protein activity, FNTB protein activity, DARS2 protein activity, TFRC protein activity, SLC7A6OS protein activity, GNB2L1 protein activity, GFER protein activity, ATP6AP2 protein activity, SLC25A19 protein activity, and/or PEAR1 protein activity).

In some embodiments of methods for overcoming or preventing resistance of a KRAS mutant cancer cell to growth inhibition and/or cell death induction by an SHP2 inhibitor or for enhancing sensitivity of a KRAS mutant cancer cell to an SHP2 inhibitor, the method comprises inhibiting in a KRAS mutant cancer cell the expression or function of one or more proteins selected from, e.g., VRK1, RIOK2, ELP4, and ELP5. In some embodiments, the method comprises inhibiting in the KRAS mutant cancer cell the function of one or more proteins selected from, TFRC, SLC7A6OS, GNB2L1, GFER, ATP6AP2, SLC25A19, and/or PEAR1.

In various embodiments of any of the above-described methods, the expression or function of an SHP2 protein described herein may be inhibited in a KRAS mutant cancer cell described herein by an SHP2 inhibitor described herein. As a non-limiting example, SHP2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SHP2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of VRK1 protein. As a non-limiting example, VRK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, VRK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RIOK2 protein. As a non-limiting example, RIOK2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RIOK2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP4 protein. As a non-limiting example, ELP4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ELP5 protein. As a non-limiting example, ELP5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ELP5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ENO1 protein. As a non-limiting example, ENO1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ENO1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GAPDH protein. As a non-limiting example, GAPDH protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GAPDH protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MARS2 protein. As a non-limiting example, MARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ATP6V1F protein. As a non-limiting example, ATP6V1F protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ATP6V1F protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PRMT5 protein. As a non-limiting example, PRMT5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PRMT5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of COQ2 protein. As a non-limiting example, COQ2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, COQ2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DBR1 protein. As a non-limiting example, DBR1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DBR1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DTYMK protein. As a non-limiting example, DTYMK protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DTYMK protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DKC1 protein. As a non-limiting example, DKC1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DKC1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RNMT protein. As a non-limiting example, RNMT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RNMT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PPP1R8 protein. As a non-limiting example, PPP1R8 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PPP1R8 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of HSD17B10 protein. As a non-limiting example, HSD17B10 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, HSD17B10 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DOLK protein. As a non-limiting example, DOLK protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DOLK protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ALG1 protein. As a non-limiting example, ALG1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ALG1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UROD protein. As a non-limiting example, UROD protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UROD protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POLR3H protein. As a non-limiting example, POLR3H protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POLR3H protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PGD protein. As a non-limiting example, PGD protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PGD protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSEN2 protein. As a non-limiting example, TSEN2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSEN2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RNASEH2A protein. As a non-limiting example, RNASEH2A protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RNASEH2A protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GUK1 protein. As a non-limiting example, GUK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GUK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSFM protein. As a non-limiting example, TSFM protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSFM protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NELFB protein. As a non-limiting example, NELFB protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NELFB protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DOHH protein. As a non-limiting example, DOHH protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DOHH protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EXOSC5 protein. As a non-limiting example, EXOSC5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EXOSC5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPE protein. As a non-limiting example, RPE protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPE protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CSTF1 protein. As a non-limiting example, CSTF1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CSTF1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RTEL1 protein. As a non-limiting example, RTEL1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RTEL1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of WARS2 protein. As a non-limiting example, WARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, WARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UTP23 protein. As a non-limiting example, UTP23 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UTP23 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POLG2 protein. As a non-limiting example, POLG2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POLG2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of THG1L protein. As a non-limiting example, THG1L protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, THG1L protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RARS2 protein. As a non-limiting example, RARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RAD51D protein. As a non-limiting example, RAD51D protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RAD51D protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of LARS2 protein. As a non-limiting example, LARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SDHB protein. As a non-limiting example, SDHB protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SDHB protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CPSF4 protein. As a non-limiting example, CPSF4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CPSF4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PDPK1 protein. As a non-limiting example, PDPK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PDPK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX10 protein. As a non-limiting example, DDX10 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX10 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of VARS2 protein. As a non-limiting example, VARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, VARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PDSS2 protein. As a non-limiting example, PDSS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PDSS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PSMG4 protein. As a non-limiting example, PSMG4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PSMG4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DHX33 protein. As a non-limiting example, DHX33 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DHX33 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of COASY protein. As a non-limiting example, COASY protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, COASY protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of VHL protein. As a non-limiting example, VHL protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, VHL protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RNGTT protein. As a non-limiting example, RNGTT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RNGTT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PPP1R2 protein. As a non-limiting example, PPP1R2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PPP1R2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NOL11 protein. As a non-limiting example, NOL11 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NOL11 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CTDNEP1 protein. As a non-limiting example, CTDNEP1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CTDNEP1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ISG20L2 protein. As a non-limiting example, ISG20L2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ISG20L2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ERCC2 protein. As a non-limiting example, ERCC2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ERCC2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TOP3A protein. As a non-limiting example, TOP3A protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TOP3A protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MTG2 protein. As a non-limiting example, MTG2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MTG2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of BRF1 protein. As a non-limiting example, BRF1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, BRF1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PIK3C3 protein. As a non-limiting example, PIK3C3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PIK3C3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IARS protein. As a non-limiting example, IARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of AURKAIP1 protein. As a non-limiting example, AURKAIP1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, AURKAIP1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UQCRFS1 protein. As a non-limiting example, UQCRFS1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UQCRFS1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PRMT1 protein. As a non-limiting example, PRMT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PRMT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX59 protein. As a non-limiting example, DDX59 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX59 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MARS protein. As a non-limiting example, MARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TOE1 protein. As a non-limiting example, TOE1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TOE1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SARS2 protein. As a non-limiting example, SARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CDIPT protein. As a non-limiting example, CDIPT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CDIPT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of YARS protein. As a non-limiting example, YARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, YARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CARS2 protein. As a non-limiting example, CARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PPP2R4 protein. As a non-limiting example, PPP2R4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PPP2R4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPP21 protein. As a non-limiting example, RPP21 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPP21 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UGP2 protein. As a non-limiting example, UGP2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UGP2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DPAGT1 protein. As a non-limiting example, DPAGT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DPAGT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PYROXD1 protein. As a non-limiting example, PYROXD1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PYROXD1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MTOR protein. As a non-limiting example, MTOR protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MTOR protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of HARS2 protein. As a non-limiting example, HARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, HARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NARS protein. As a non-limiting example, NARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSC1 protein. As a non-limiting example, TSC1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSC1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POLR3C protein. As a non-limiting example, POLR3C protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POLR3C protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of QRSL1 protein. As a non-limiting example, QRSL1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, QRSL1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPIA protein. As a non-limiting example, RPIA protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPIA protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SDHC protein. As a non-limiting example, SDHC protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SDHC protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX56 protein. As a non-limiting example, DDX56 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX56 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EIF4E protein. As a non-limiting example, EIF4E protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EIF4E protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX46 protein. As a non-limiting example, DDX46 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX46 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IMPDH2 protein. As a non-limiting example, IMPDH2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IMPDH2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SOD2 protein. As a non-limiting example, SOD2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SOD2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UBE2M protein. As a non-limiting example, UBE2M protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UBE2M protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GATC protein. As a non-limiting example, GATC protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GATC protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSC2 protein. As a non-limiting example, TSC2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSC2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PMPCA protein. As a non-limiting example, PMPCA protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PMPCA protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TSEN54 protein. As a non-limiting example, TSEN54 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TSEN54 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of FOXM1 protein. As a non-limiting example, FOXM1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, FOXM1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of FARS2 protein. As a non-limiting example, FARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, FARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CTPS1 protein. As a non-limiting example, CTPS1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CTPS1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PARS2 protein. As a non-limiting example, PARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ALG2 protein. As a non-limiting example, ALG2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ALG2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EIF2B3 protein. As a non-limiting example, EIF2B3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EIF2B3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CMPK1 protein. As a non-limiting example, CMPK1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CMPK1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DHDDS protein. As a non-limiting example, DHDDS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DHDDS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SAE1 protein. As a non-limiting example, SAE1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SAE1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NARS2 protein. As a non-limiting example, NARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PNKP protein. As a non-limiting example, PNKP protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PNKP protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PDSS1 protein. As a non-limiting example, PDSS1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PDSS1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POLR3K protein. As a non-limiting example, POLR3K protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POLR3K protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of AHCY protein. As a non-limiting example, AHCY protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, AHCY protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NAE1 protein. As a non-limiting example, NAE1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NAE1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UBIAD1 protein. As a non-limiting example, UBIAD1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UBIAD1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RPUSD4 protein. As a non-limiting example, RPUSD4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RPUSD4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell expression or function of EARS2 protein. As a non-limiting example, EARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GMPPB protein. As a non-limiting example, GMPPB protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GMPPB protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of LIAS protein. As a non-limiting example, LIAS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LIAS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PPP4C protein. As a non-limiting example, PPP4C protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PPP4C protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NSUN4 protein. As a non-limiting example, NSUN4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NSUN4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DLD protein. As a non-limiting example, DLD protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DLD protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TRMT5 protein. As a non-limiting example, TRMT5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TRMT5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of AASDHPPT protein. As a non-limiting example, AASDHPPT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, AASDHPPT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EIF5A protein. As a non-limiting example, EIF5A protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EIF5A protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POT1 protein. As a non-limiting example, POT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DHX9 protein. As a non-limiting example, DHX9 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DHX9 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of LONP1 protein. As a non-limiting example, LONP1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, LONP1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PPP6C protein. As a non-limiting example, PPP6C protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PPP6C protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SKIV2L2 protein. As a non-limiting example, SKIV2L2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SKIV2L2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PTDSS1 protein. As a non-limiting example, PTDSS1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PTDSS1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of USP5 protein. As a non-limiting example, USP5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, USP5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of VPS52 protein. As a non-limiting example, VPS52 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, VPS52 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TKT protein. As a non-limiting example, TKT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TKT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TRMT61A protein. As a non-limiting example, TRMT61A protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TRMT61A protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of N6AMT1 protein. As a non-limiting example, N6AMT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, N6AMT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GGPS1 protein. As a non-limiting example, GGPS1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GGPS1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EFTUD1 protein. As a non-limiting example, EFTUD1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EFTUD1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ACAD9 protein. As a non-limiting example, ACAD9 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ACAD9 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SETD1A protein. As a non-limiting example, SETD1A protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SETD1A protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IPO11 protein. As a non-limiting example, IPO11 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IPO11 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EIF3I protein. As a non-limiting example, EIF3I protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EIF3I protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of METTL16 protein. As a non-limiting example, METTL16 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, METTL16 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MASTL protein. As a non-limiting example, MASTL protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MASTL protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX51 protein. As a non-limiting example, DDX51 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX51 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ADAT3 protein. As a non-limiting example, ADAT3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ADAT3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ZNRD1 protein. As a non-limiting example, ZNRD1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ZNRD1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of OGT protein. As a non-limiting example, OGT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, OGT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IDI1 protein. As a non-limiting example, IDI1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IDI1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IMP4 protein. As a non-limiting example, IMP4 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IMP4 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of FTSJ3 protein. As a non-limiting example, FTSJ3 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, FTSJ3 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EXOSC8 protein. As a non-limiting example, EXOSC8 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EXOSC8 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GSG2 protein. As a non-limiting example, GSG2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GSG2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PI4KA protein. As a non-limiting example, PI4KA protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PI4KA protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of NSMCE2 protein. As a non-limiting example, NSMCE2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, NSMCE2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDX52 protein. As a non-limiting example, DDX52 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDX52 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DDOST protein. As a non-limiting example, DDOST protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DDOST protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CSNK2B protein. As a non-limiting example, CSNK2B protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CSNK2B protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UBA2 protein. As a non-limiting example, UBA2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UBA2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RABGGTA protein. As a non-limiting example, RABGGTA protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RABGGTA protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SOD1 protein. As a non-limiting example, SOD1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SOD1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TRIT1 protein. As a non-limiting example, TRIT1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TRIT1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TYMS protein. As a non-limiting example, TYMS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TYMS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RNF168 protein. As a non-limiting example, RNF168 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RNF168 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of UBE2I protein. As a non-limiting example, UBE2I protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, UBE2I protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GARS protein. As a non-limiting example, GARS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GARS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of IPO13 protein. As a non-limiting example, IPO13 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, IPO13 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SMARCB1 protein. As a non-limiting example, SMARCB1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SMARCB1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of EIF2B1 protein. As a non-limiting example, EIF2B1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, EIF2B1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of RNASEH1 protein. As a non-limiting example, RNASEH1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, RNASEH1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of MCAT protein. As a non-limiting example, MCAT protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, MCAT protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of XRN2 protein. As a non-limiting example, XRN2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, XRN2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of POP5 protein. As a non-limiting example, POP5 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, POP5 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of CS protein. As a non-limiting example, CS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, CS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of FNTB protein. As a non-limiting example, FNTB protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, FNTB protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of DARS2 protein. As a non-limiting example, DARS2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, DARS2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of TFRC protein. As a non-limiting example, TFRC protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, TFRC protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SLC7A6OS protein. As a non-limiting example, SLC7A6OS protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SLC7A6OS protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GNB2L1 protein. As a non-limiting example, GNB2L1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GNB2L1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of GFER protein. As a non-limiting example, GFER protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, GFER protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of ATP6AP2 protein. As a non-limiting example, ATP6AP2 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, ATP6AP2 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of SLC25A19 protein. As a non-limiting example, SLC25A19 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, SLC25A19 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments of any of the above-described methods, the method comprises inhibiting in the KRAS mutant cancer cell the expression or function of PEAR1 protein. As a non-limiting example, PEAR1 protein expression or function may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, PEAR1 protein expression or function may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, a method of the present disclosure (e.g., a method for overcoming or preventing resistance of a KRAS mutant cancer cell to growth inhibition and/or cell death induction by an SHP2 inhibitor or a method for enhancing sensitivity of a KRAS mutant cancer cell to an SHP2 inhibitor) comprises binding on the surface of the KRAS mutant cancer cell one or more proteins selected from TFRC, SLC7A6OS, GNB2L1, GFER, ATP6AP2, SLC25A19, and PEAR1 with a binding partner, wherein said binding partner specifically binds to the one or more proteins. In some embodiments, the binding partner is capable of inhibiting in the KRAS mutant cancer cell function of one or more proteins selected from TFRC, SLC7A6OS, GNB2L1, GFER, ATP6AP2, SLC25A19, and PEAR1.

In various embodiments, the activity of TFRC protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of TFRC protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of SLC7A6OS protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of SLC7A6OS protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of GNB2L1 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of GNB2L1 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of GFER protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of GFER protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of ATP6AP2 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of ATP6AP2 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of SLC25A19 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of SLC25A19 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the activity of PEAR1 protein may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the activity of PEAR1 protein may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments of any of the above-described methods, the method comprises administering to a KRAS mutant cancer cell an inhibitor of the expression or function of the one or more proteins described herein (e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1) or a degrader of the one or more proteins described herein (e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1).

In some embodiments, the inhibitor of the expression or function of the one or more proteins or the degrader of the one or more proteins described herein is administered to the KRAS mutant cancer cell simultaneously or sequentially with the SHP2 inhibitor. As a non-limiting example, the inhibitor of the expression or function of the one or more proteins or the degrader of the one or more proteins described herein can be administered to the KRAS mutant cancer cell simultaneously with the SHP2 inhibitor in one composition. As another non-limiting example, the inhibitor of the expression or function of the one or more proteins or the degrader of the one or more proteins described herein can be administered to the KRAS mutant cancer cell simultaneously with the SHP2 inhibitor in different compositions. As yet another non-limiting example, the inhibitor of the expression or function of the one or more proteins or the degrader of the one or more proteins described herein can be administered to the KRAS mutant cancer cell sequentially with the SHP2 inhibitor in different compositions.

In some embodiments, when the inhibitor of the expression or function of the one or more proteins or the degrader of the one or more proteins described herein and the SHP2 inhibitor are administered to the KRAS mutant cancer cell sequentially (e.g., in different compositions), the inhibitor of the expression or function of the one or more proteins or the degrader of the one or more proteins may be administered as a first component of a dosing regimen and the SHP2 inhibitor may be administered as a second component of a dosing regimen (i.e., the inhibitor of the expression or function of the one or more proteins or the degrader of the one or more proteins may be administered before the SHP2 inhibitor).

In some embodiments, when the inhibitor of the expression or function of the one or more proteins or the degrader of the one or more proteins described herein and the SHP2 inhibitor are administered to the KRAS mutant cancer cell sequentially (e.g., in different compositions), the SHP2 inhibitor may be administered as a first component of a dosing regimen and the inhibitor of the expression or function of the one or more proteins or the degrader of the one or more proteins may be administered as a second component of a dosing regimen (i.e., the SHP2 inhibitor may be administered before the inhibitor of the expression or function of the one or more proteins or the degrader of the one or more proteins).

In some embodiments, the binding partner of said one or more proteins is administered to the KRAS mutant cancer cell. In further embodiments, said binding partner of said one or more proteins is administered to the KRAS mutant cancer cell simultaneously or sequentially with the SHP2 inhibitor.

In some embodiments, the binding partner of said one or more proteins comprises an intact antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a single-chain variable fragment (scFv), a Bi-specific T-cell engager (BiTE), a bispecific killer cell engager (BiKE), a CrossMab, a tri-specific binding partner, or a chimeric antigen receptor (CAR). Further, in some embodiments, the binding partner of said one or more proteins is conjugated to a detectable label, a chemotherapeutic agent, a radioisotope, or a toxin. In some embodiments, the binding partner of said one or more proteins is a component of a fusion protein. In some embodiments, the binding partner of said one or more proteins comprises a chimeric antigen receptor (CAR). In some embodiments, the binding partner of said one or more proteins is expressed by a T cell or a natural killer cell.

In some embodiments, a KRAS polynucleotide (e.g., DNA or RNA) comprises one or more mutations. In certain embodiments, the KRAS polynucleotide comprises one or more mutations in exon 1 at codons 12 or 13. In some embodiments, the KRAS polynucleotide comprises one or multiple mutations at codons 18, 61, 63, 117, 119, or 146. In some embodiments, the KRAS polynucleotide comprises one or more mutations at positions that correspond to amino acid residues 12, 13, 18, 19, 20, 22, 24, 26, 36, 59, 61, 63, 64, 68, 110, 116, 117, 119, 146, 147, 158, 164, 176, or a combination thereof. In certain embodiments, KRAS polynucleotide comprises one or multiple mutations at positions that correspond to amino acid residues G12V, G12D, G12C, G12A, G12S, G12F, G12R, G12L, G12T, G13C, G13D, G13V, G13R, G13H, G13A, Q61K, Q61H, Q61L, Q61R, Q61P, Q61E, E62K, E63K, R68S, R68G, R68M, D69G, V14G, V14I, S17G, A18D, A146T, A146P, A146V, A146G, L19F, T20M, T2OR, I21R, Q22K, I24N, I24V, T35A, I36L, I36M, T50I, D57N, T58I, N26K, H27N, D33E, P34L, P34R, A59G, A59S, A59T, A59E, G60V, Y71D, Y71C, Y64D, Y64N, Y64H, Y96C, Y96D, M72V, M72T, T74P, D92Y, P110S, N116H, N116S, K117N, K117E, K117R, K147N, C118S, Q131H, R135T, R161G, R164Q, T144I, D153V, A155D, F156L, T158A, K176Q, or a combination thereof.

In some aspects, a KRAS mutation can comprise a mutation in one or multiple codons in the KRAS gene. In certain aspects, the KRAS mutation refers to a mutation at, for example, without limitation, codon 12, at codon 13, at codon 61, or at codon 117, or a combination thereof.

In some embodiments, a KRAS mutation can comprise a mutation in one or multiple amino acids in the KRAS protein. Examples of amino acid mutations comprise, but are not limited to, amino acid substitutions, deletions, and/or insertions. Amino acid substitution means that an amino acid residue is substituted (i.e., replaced) for a different amino acid residue at the same position. Amino acid deletion means that an amino acid residue is deleted (i.e., removed). Amino acid residues that are inserted may be inserted at any position and may be added such that some or all of the inserted amino acid residues are immediately adjacent to one another or may be added such that none of the inserted amino acid residues are immediately adjacent to one another. In some aspects, a KRAS mutation refers to, e.g., a G12 mutation, a G13 mutation, a H61 mutation, or a K117 mutation, or a combination thereof. In certain aspects, the KRAS mutation refers to a G12C mutation, a G12V mutation, a G12D mutation, a G13D mutation, a Q61H mutation, a Q61L mutation, or a Q61R mutation, or a combination thereof.

In some embodiments, a KRAS mutant cancer cell described herein may comprise any number of various mutations in KRAS described herein, or combinations thereof. For instance, without limitation, a KRAS mutant cancer cell may comprise a mutation such as, but not limited to, a KRAS G12 mutation, a KRAS G13 mutation, a KRAS H61 mutation, or a KRAS K117 mutation. In some embodiments, when the KRAS mutant cancer cell described herein comprises a KRAS G12 mutation, the KRAS G12 mutation may be, e.g., a G12C mutation, a G12V mutation, or a G12D mutation.

In some embodiments, a KRAS mutant cancer cell described herein comprises a KRAS G12C mutation.

In various embodiments, the KRAS mutant cancer cell comprises a KRAS G13D mutation. In various embodiments, the KRAS mutant cancer cell comprises a KRAS H61 mutation. Non-limiting examples of a KRAS H61 mutation include a Q61H mutation, a Q61L mutation, and a Q61R mutation.

In some embodiments, the KRAS mutant cancer cell described herein comprises a KRAS K117N mutation.

In some embodiments, a KRAS mutant cancer cell comprising any of the above-described mutations in KRAS may, in addition, also comprise a mutation, e.g., in STK11 (also called Liver kinase B1 [LKB1]) gene and/or Kelch Like ECH Associated Protein 1 (KEAP1) gene.

KRAS mutant cancer cells of the present disclosure can be any KRAS mutant cell type known to those of skill in the art. As an example, without limitation, KRAS mutant cancer(s) cells can be liver cells (e.g., hepatocytes), stomach cells (e.g., parietal cells, endocrine cells, chief cells, mucous cells), intestinal cells (e.g., epithelial cells) including colon cells and cells of the rectum, kidney cells (e.g., endothelial cells, interstitial cells, immune cells), cardiac cells (e.g., myocardial contractile cells, myocardial conducting cells), brain cells (e.g., glia, neurons), lung cells (e.g., epithelial cells), ovarian cells, breast cells, prostate cells (e.g., basal, neuroendocrine, luminal), bladder cells (e.g., urothelial cells), blood cells (e.g., red blood cells, white blood cells, platelets), pancreatic cells (e.g., cells that line the duct of the pancreas, exocrine cells, neuroendocrine cells), or cells of the lymph system (e.g., B lymphocytes, T lymphocytes, natural killer cells). In some embodiments, the KRAS mutant cancer cell is in a subject (e.g., a human, a veterinary animal, or an experimental model).

A KRAS mutant cancer cell described herein may be derived from a KRAS mutant cancer. A KRAS mutant cancer can be, e.g., without limitation, any cancer of the present disclosure. In certain embodiments, the KRAS mutant cancer is lung cancer, colorectal cancer, or pancreatic cancer. In some embodiments, the lung cancer is non-small cell lung cancer. Additional non-limiting examples of cancers are discussed below.

Non-limiting examples of SHP2 inhibitors which may be used in the practice of the present disclosure include BBP-398, TNO155, RMC-4630, JAB-3068, RLY-1971, and ERAS-601, pharmaceutically acceptable salts thereof, and any combinations thereof.

In some embodiments, a KRAS mutant cancer cell may be in a subject. In some embodiments, the subject is human. In some embodiments, the subject is a veterinary animal (e.g., cats, dogs, cows, horses, sheep, pigs, etc.) or an experimental animal model.

### Therapeutic Methods Comprising SHP2 Inhibitors

In one aspect, the present disclosure provides a method of treating a KRAS mutant cancer in a subject in need thereof. In certain aspects, the method of treating a KRAS mutant cancer in a subject in need thereof comprises administering to the subject an effective amount of an SHP2 inhibitor and an inhibitor of the expression or function or a degrader of one or more proteins such as, but not limited to, e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In various embodiments of the above-described method, the method comprises administering to the subject an effective amount of an SHP2 inhibitor and an inhibitor of the expression or function or a degrader of one or more proteins selected from, e.g., VRK1, RIOK2, ELP4, and ELP5.

In various embodiments of any of the above-described methods, expression or function of an SHP2 protein described herein may be inhibited in a subject in need thereof by administering to the subject an effective amount of an SHP2 inhibitor described herein. As a non-limiting example, the SHP2 inhibitor may inhibit SHP2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the SHP2 inhibitor may inhibit SHP2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of VRK1 protein may be administered to a subject in need thereof. As a non-limiting example, the inhibitor of the expression or function of VRK1 protein may inhibit VRK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of VRK1 protein may inhibit VRK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of VRK1 protein may degrade VRK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of VRK1 protein may degrade VRK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RIOK2 protein may be administered to a subject in need thereof. As a non-limiting example, the inhibitor of the expression or function of RIOK2 protein may inhibit RIOK2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RIOK2 protein may inhibit RIOK2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RIOK2 protein may degrade RIOK2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RIOK2 protein may degrade RIOK2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ELP4 protein may be administered to a subject in need thereof. As a non-limiting example, the inhibitor of the expression or function of ELP4 protein may inhibit ELP4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP4 protein may inhibit ELP4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP4 protein may degrade ELP4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP4 protein may degrade ELP4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ELP5 protein may be administered to a subject in need thereof. As a non-limiting example, the inhibitor of the expression or function of ELP5 protein may inhibit ELP5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ELP5 protein may inhibit ELP5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ELP5 protein may degrade ELP5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ELP5 protein may degrade ELP5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ENO1 protein may be administered to a subject in need thereof. As a non-limiting example, the inhibitor of the expression or function of ENO1 protein may inhibit ENO1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ENO1 protein may inhibit ENO1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ENO1 protein may degrade ENO1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ENO1 protein may degrade ENO1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GAPDH protein may be administered to a subject in need thereof. As a non-limiting example, the inhibitor of the expression or function of GAPDH protein may inhibit GAPDH protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GAPDH protein may inhibit GAPDH protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GAPDH protein may degrade GAPDH protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GAPDH protein may degrade GAPDH protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MARS2 protein may be administered to a subject in need thereof. As a non-limiting example, the inhibitor of the expression or function of MARS2 protein may inhibit MARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MARS2 protein may inhibit MARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MARS2 protein may degrade MARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MARS2 protein may degrade MARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ATP6V1F protein may be administered to a subject in need thereof. As a non-limiting example, the inhibitor of the expression or function of ATP6V1F protein may inhibit ATP6V1F protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ATP6V1F protein may inhibit ATP6V1F protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ATP6V1F protein may degrade ATP6V1F protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ATP6V1F protein may degrade ATP6V1F protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PRMT5 protein may be administered to a subject in need thereof. As a non-limiting example, the inhibitor of the expression or function of PRMT5 protein may inhibit PRMT5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PRMT5 protein may inhibit PRMT5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PRMT5 protein may degrade PRMT5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PRMT5 protein may degrade PRMT5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of COQ2 protein may be administered to a subject in need thereof. As a non-limiting example, the inhibitor of the expression or function of COQ2 protein may inhibit COQ2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of COQ2 protein may inhibit COQ2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of COQ2 protein may degrade COQ2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of COQ2 protein may degrade COQ2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DBR1 protein may be administered to a subject in need thereof. As a non-limiting example, the inhibitor of the expression or function of DBR1 protein may inhibit DBR1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor of the expression or function of DBR1 protein may inhibit DBR1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DBR1 protein may degrade DBR1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DBR1 protein may degrade DBR1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DTYMK protein may be administered to a subject in need thereof. As a non-limiting example, the inhibitor of the expression or function of DTYMK protein may inhibit DTYMK protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DTYMK protein may inhibit DTYMK protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DTYMK protein may degrade DTYMK protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DTYMK protein may degrade DTYMK protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DKC1 protein may be administered to a subject in need thereof. As a non-limiting example, the inhibitor of the expression or function of DKC1 protein may inhibit DKC1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DKC1 protein may inhibit DKC1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DKC1 protein may degrade DKC1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DKC1 protein may degrade DKC1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of the inhibitor of expression or function and/or a degrader of RNMT protein may be administered to a subject in need thereof. As a non-limiting example, the inhibitor of the expression or function of RNMT protein may inhibit RNMT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RNMT protein may inhibit RNMT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RNMT protein may degrade RNMT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RNMT protein may degrade RNMT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PPP1R8 protein may be administered to a subject in need thereof. As a non-limiting example, the inhibitor of the expression or function of PPP1R8 protein may inhibit PPP1R8 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PPP1R8 protein may inhibit PPP1R8 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PPP1R8 protein may degrade PPP1R8 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PPP1R8 protein may degrade PPP1R8 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of HSD17B10 protein may be administered to a subject in need thereof. As a non-limiting example, the inhibitor of the expression or function of HSD17B10 protein may inhibit HSD17B10 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of HSD17B10 protein may inhibit HSD17B10 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of HSD17B10 protein may degrade HSD17B10 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of HSD17B10 protein may degrade HSD17B10 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DOLK protein may be administered to a subject in need thereof. As a non-limiting example, the inhibitor of the expression or function of DOLK protein may inhibit DOLK protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DOLK protein may inhibit DOLK protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DOLK protein may degrade DOLK protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DOLK protein may degrade DOLK protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ALG1 protein may be administered to a subject in need thereof. As a non-limiting example, the inhibitor of the expression or function of ALG1 protein may inhibit ALG1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ALG1 protein may inhibit ALG1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ALG1 protein may degrade ALG1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ALG1 protein may degrade ALG1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UROD protein may be administered to a subject in need thereof. As a non-limiting example, the inhibitor of the expression or function of UROD protein may inhibit UROD protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UROD protein may inhibit UROD protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UROD protein may degrade UROD protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UROD protein may degrade UROD protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POLR3H protein may be administered to a subject in need thereof. As a non-limiting example, the inhibitor of the expression or function of POLR3H protein may inhibit POLR3H protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POLR3H protein may inhibit POLR3H protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POLR3H protein may degrade POLR3H protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POLR3H protein may degrade POLR3H protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PGD protein may be administered to a subject in need thereof. As a non-limiting example, the inhibitor of the expression or function of PGD protein may inhibit PGD protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PGD protein may inhibit PGD protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PGD protein may degrade PGD protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PGD protein may degrade PGD protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TSEN2 protein may be administered to a subject in need thereof. As a non-limiting example, the inhibitor of the expression or function of TSEN2 protein may inhibit TSEN2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSEN2 protein may inhibit TSEN2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSEN2 protein may degrade TSEN2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSEN2 protein may degrade TSEN2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RNASEH2A protein may be administered to a subject in need thereof. As a non-limiting example, the inhibitor of the expression or function of RNASEH2A protein may inhibit RNASEH2A protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RNASEH2A protein may inhibit RNASEH2A protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RNASEH2A protein may degrade RNASEH2A protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RNASEH2A protein may degrade RNASEH2A protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GUK1 protein may be administered to a subject in need thereof. As a non-limiting example, the inhibitor of the expression or function of GUK1 protein may inhibit GUK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GUK1 protein may inhibit GUK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GUK1 protein may degrade GUK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GUK1 protein may degrade GUK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TSFM protein may be administered to a subject in need thereof. As a non-limiting example, the inhibitor of the expression or function of TSFM protein may inhibit TSFM protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSFM protein may inhibit TSFM protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSFM protein may degrade TSFM protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSFM protein may degrade TSFM protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NELFB protein may be administered to a subject in need thereof. As a non-limiting example, the inhibitor of the expression or function of NELFB protein may inhibit NELFB protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NELFB protein may inhibit NELFB protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NELFB protein may degrade NELFB protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NELFB protein may degrade NELFB protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DOHH protein may be administered to a subject in need thereof. As a non-limiting example, the inhibitor of the expression or function of DOHH protein may inhibit DOHH protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DOHH protein may inhibit DOHH protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DOHH protein may degrade DOHH protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DOHH protein may degrade DOHH protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EXOSC5 protein may be administered to a subject in need thereof. As a non-limiting example, the inhibitor of the expression or function of EXOSC5 protein may inhibit EXOSC5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EXOSC5 protein may inhibit EXOSC5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EXOSC5 protein may degrade EXOSC5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EXOSC5 protein may degrade EXOSC5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RPE protein may be administered to a subject in need thereof. As a non-limiting example, the inhibitor of the expression or function of RPE protein may inhibit RPE protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPE protein may inhibit RPE protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPE protein may degrade RPE protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPE protein may degrade RPE protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CSTF1 protein may be administered to a subject in need thereof. As a non-limiting example, the inhibitor of the expression or function of CSTF1 protein may inhibit CSTF1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CSTF1 protein may inhibit CSTF1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CSTF1 protein may degrade CSTF1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CSTF1 protein may degrade CSTF1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RTEL1 protein may be administered to a subject in need thereof. As a non-limiting example, the inhibitor of the expression or function of RTEL1 protein may inhibit RTEL1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of expression or function of RTEL1 protein may inhibit RTEL1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RTEL1 protein may degrade RTEL1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RTEL1 protein may degrade RTEL1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of WARS2 protein may be administered to a subject in need thereof. As a non-limiting example, the inhibitor of the expression or function of WARS2 protein may inhibit WARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of WARS2 protein may inhibit WARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of WARS2 protein may degrade WARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of WARS2 protein may degrade WARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UTP23 protein may be administered to a subject in need thereof. As a non-limiting example, the inhibitor of the expression or function of UTP23 protein may inhibit UTP23 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UTP23 protein may inhibit UTP23 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UTP23 protein may degrade UTP23 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UTP23 protein may degrade UTP23 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POLG2 protein may be administered to a subject in need thereof. As a non-limiting example, the inhibitor of the expression or function of POLG2 protein may inhibit POLG2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POLG2 protein may inhibit POLG2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POLG2 protein may degrade POLG2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POLG2 protein may degrade POLG2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of THG1L protein may be administered to a subject in need thereof. As a non-limiting example, the inhibitor of the expression or function of THG1L protein may inhibit THG1L protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of expression or function of THG1L protein may inhibit THG1L protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of THG1L protein may degrade THG1L protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of THG1L protein may degrade THG1L protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RARS2 protein may be administered to a subject in need thereof. As a non-limiting example, the inhibitor of the expression or function of RARS2 protein may inhibit RARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RARS2 protein may inhibit RARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RARS2 protein may degrade RARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RARS2 protein may degrade RARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RAD51D protein may be administered to a subject in need thereof. As a non-limiting example, the inhibitor of the expression or function of RAD51D protein may inhibit RAD51D protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RAD51D protein may inhibit RAD51D protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RAD51D protein may degrade RAD51D protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RAD51D protein may degrade RAD51D protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of LARS2 protein may be administered to a subject in need thereof. As a non-limiting example, the inhibitor of the expression or function of LARS2 protein may inhibit LARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of LARS2 protein may inhibit LARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of LARS2 protein may degrade LARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of LARS2 protein may degrade LARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SDHB protein may be administered to a subject in need thereof. As a non-limiting example, the inhibitor of the expression or function of SDHB protein may inhibit SDHB protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SDHB protein may inhibit SDHB protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SDHB protein may degrade SDHB protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SDHB protein may degrade SDHB protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CPSF4 protein may be administered to a subject in need thereof. As a non-limiting example, the inhibitor of the expression or function of CPSF4 protein may inhibit CPSF4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CPSF4 protein may inhibit CPSF4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CPSF4 protein may degrade CPSF4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CPSF4 protein may degrade CPSF4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PDPK1 protein may be administered to a subject in need thereof. As a non-limiting example, the inhibitor of the expression or function of PDPK1 protein may inhibit PDPK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PDPK1 protein may inhibit PDPK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PDPK1 protein may degrade PDPK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PDPK1 protein may degrade PDPK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX10 protein may be administered to a subject in need thereof. As a non-limiting example, the inhibitor of the expression or function of DDX10 protein may inhibit DDX10 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX10 protein may inhibit DDX10 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX10 protein may degrade DDX10 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX10 protein may degrade DDX10 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of VARS2 protein may be administered to a subject in need thereof. As a non-limiting example, the inhibitor of the expression or function of VARS2 protein may inhibit VARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of VARS2 protein may inhibit VARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of VARS2 protein may degrade VARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of VARS2 protein may degrade VARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PDSS2 protein may be administered to a subject in need thereof. As a non-limiting example, the inhibitor of the expression or function of PDSS2 protein may inhibit PDSS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PDSS2 protein may inhibit PDSS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PDSS2 protein may degrade PDSS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PDSS2 protein may degrade PDSS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PSMG4 protein may be administered to a subject in need thereof. As a non-limiting example, the inhibitor of the expression or function of PSMG4 protein may inhibit PSMG4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PSMG4 protein may inhibit PSMG4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PSMG4 protein may degrade PSMG4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PSMG4 protein may degrade PSMG4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DHX33 protein may be administered to a subject in need thereof. As a non-limiting example, the inhibitor of the expression or function of DHX33 protein may inhibit DHX33 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DHX33 protein may inhibit DHX33 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DHX33 protein may degrade DHX33 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DHX33 protein may degrade DHX33 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of COASY protein may be administered to a subject in need thereof. As a non-limiting example, the inhibitor of the expression or function of COASY protein may inhibit COASY protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of COASY protein may inhibit COASY protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of COASY protein may degrade COASY protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of COASY protein may degrade COASY protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of VHL protein may be administered to a subject in need thereof. As a non-limiting example, the inhibitor of the expression or function of VHL protein may inhibit VHL protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of VHL protein may inhibit VHL protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of VHL protein may degrade VHL protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of VHL protein may degrade VHL protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RNGTT protein may be administered to a subject in need thereof. As a non-limiting example, the inhibitor of the expression or function of RNGTT protein may inhibit RNGTT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RNGTT protein may inhibit RNGTT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RNGTT protein may degrade RNGTT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RNGTT protein may degrade RNGTT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PPP1R2 protein may be administered to a subject in need thereof. As a non-limiting example, the inhibitor of the expression or function of PPP1R2 protein may inhibit PPP1R2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PPP1R2 protein may inhibit PPP1R2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PPP1R2 protein may degrade PPP1R2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PPP1R2 protein may degrade PPP1R2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NOL11 protein may be administered to a subject in need thereof. As a non-limiting example, the inhibitor of the expression or function of NOL11 protein may inhibit NOL11 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NOL11 protein may inhibit NOL11 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NOL11 protein may degrade NOL11 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NOL11 protein may degrade NOL1I protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CTDNEP1 protein may be administered to a subject in need thereof. As a non-limiting example, the inhibitor of the expression or function of CTDNEP1 protein may inhibit CTDNEP1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CTDNEP1 protein may inhibit CTDNEP1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CTDNEP1 protein may degrade CTDNEP1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CTDNEP1 protein may degrade CTDNEP1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ISG20L2 protein may be administered to a subject in need thereof. As a non-limiting example, the inhibitor of the expression or function of ISG20L2 protein may inhibit ISG20L2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ISG20L2 protein may inhibit ISG20L2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ISG20L2 protein may degrade ISG20L2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ISG20L2 protein may degrade ISG20L2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ERCC2 protein may be administered to a subject in need thereof. As a non-limiting example, the inhibitor of the expression or function of ERCC2 protein may inhibit ERCC2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ERCC2 protein may inhibit ERCC2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ERCC2 protein may degrade ERCC2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ERCC2 protein may degrade ERCC2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TOP3A protein may be administered to a subject in need thereof. As a non-limiting example, the inhibitor of the expression or function of TOP3A protein may inhibit TOP3A protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TOP3A protein may inhibit TOP3A protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TOP3A protein may degrade TOP3A protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TOP3A protein may degrade TOP3A protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MTG2 protein may be administered to a subject in need thereof. As a non-limiting example, the inhibitor of the expression or function of MTG2 protein may inhibit MTG2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MTG2 protein may inhibit MTG2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MTG2 protein may degrade MTG2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MTG2 protein may degrade MTG2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of BRF1 protein may be administered to a subject in need thereof. As a non-limiting example, the inhibitor of the expression or function of BRF1 protein may inhibit BRF1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of BRF1 protein may inhibit BRF1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of BRF1 protein may degrade BRF1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of BRF1 protein may degrade BRF1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PIK3C3 protein may be administered to a subject in need thereof. As a non-limiting example, the inhibitor of the expression or function of PIK3C3 protein may inhibit PIK3C3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PIK3C3 protein may inhibit PIK3C3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PIK3C3 protein may degrade PIK3C3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PIK3C3 protein may degrade PIK3C3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IARS protein may be administered to a subject in need thereof. As a non-limiting example, the inhibitor of the expression or function of IARS protein may inhibit IARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IARS protein may inhibit IARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IARS protein may degrade IARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IARS protein may degrade IARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of AURKAIP1 protein may be administered to a subject in need thereof. As a non-limiting example, the inhibitor of the expression or function of AURKAIP1 protein may inhibit AURKAIP1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of AURKAIP1 protein may inhibit AURKAIP1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of AURKAIP1 protein may degrade AURKAIP1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of AURKAIP1 protein may degrade AURKAIP1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UQCRFS1 protein may be administered to a subject in need thereof. As a non-limiting example, the inhibitor of the expression or function of UQCRFS1 protein may inhibit UQCRFS1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UQCRFS1 protein may inhibit UQCRFS1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UQCRFS1 protein may degrade UQCRFS1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UQCRFS1 protein may degrade UQCRFS1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PRMT1 protein may be administered to a subject in need thereof. As a non-limiting example, the inhibitor of the expression or function of PRMT1 protein may inhibit PRMT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PRMT1 protein may inhibit PRMT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PRMT1 protein may degrade PRMT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PRMT1 protein may degrade PRMT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX59 protein may be administered to a subject in need thereof. As a non-limiting example, the inhibitor of the expression or function of DDX59 protein may inhibit DDX59 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX59 protein may inhibit DDX59 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX59 protein may degrade DDX59 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX59 protein may degrade DDX59 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MARS protein may be administered to a subject in need thereof. As a non-limiting example, the inhibitor of the expression or function of MARS protein may inhibit MARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MARS protein may inhibit MARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MARS protein may degrade MARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MARS protein may degrade MARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TOE1 protein may be administered to a subject in need thereof. As a non-limiting example, the inhibitor of the expression or function of TOE1 protein may inhibit TOE1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TOE1 protein may inhibit TOE1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TOE1 protein may degrade TOE1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TOE1 protein may degrade TOE1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SARS2 protein may be administered to a subject in need thereof. As a non-limiting example, the inhibitor of the expression or function of SARS2 protein may inhibit SARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SARS2 protein may inhibit SARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SARS2 protein may degrade SARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SARS2 protein may degrade SARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CDIPT protein may be administered to a subject in need thereof. As a non-limiting example, the inhibitor of the expression or function of CDIPT protein may inhibit CDIPT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CDIPT protein may inhibit CDIPT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CDIPT protein may degrade CDIPT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CDIPT protein may degrade CDIPT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of YARS protein may be administered to a subject in need thereof. As a non-limiting example, the inhibitor of the expression or function of YARS protein may inhibit YARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of YARS protein may inhibit YARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of YARS protein may degrade YARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of YARS protein may degrade YARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CARS2 protein may be administered to a subject in need thereof. As a non-limiting example, the inhibitor of the expression or function of CARS2 protein may inhibit CARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CARS2 protein may inhibit CARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CARS2 protein may degrade CARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CARS2 protein may degrade CARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PPP2R4 protein may be administered to a subject in need thereof. As a non-limiting example, the inhibitor of the expression or function of PPP2R4 protein may inhibit PPP2R4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PPP2R4 protein may inhibit PPP2R4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PPP2R4 protein may degrade PPP2R4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PPP2R4 protein may degrade PPP2R4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RPP21 protein may be administered to a subject in need thereof. As a non-limiting example, the inhibitor of the expression or function of RPP21 protein may inhibit RPP21 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPP21 protein may inhibit RPP21 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPP21 protein may degrade RPP21 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPP21 protein may degrade RPP21 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UGP2 protein may be administered to a subject in need thereof. As a non-limiting example, the inhibitor of the expression or function of UGP2 protein may inhibit UGP2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UGP2 protein may inhibit UGP2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UGP2 protein may degrade UGP2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UGP2 protein may degrade UGP2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DPAGT1 protein may be administered to a subject in need thereof. As a non-limiting example, the inhibitor of the expression or function of DPAGT1 protein may inhibit DPAGT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DPAGT1 protein may inhibit DPAGT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DPAGT1 protein may degrade DPAGT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DPAGT1 protein may degrade DPAGT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PYROXD1 protein may be administered to a subject in need thereof. As a non-limiting example, the inhibitor of the expression or function of PYROXD1 protein may inhibit PYROXD1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PYROXD1 protein may inhibit PYROXD1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PYROXD1 protein may degrade PYROXD1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PYROXD1 protein may degrade PYROXD1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MTOR protein may be administered to a subject in need thereof. As a non-limiting example, the inhibitor of the expression or function of MTOR protein may inhibit MTOR protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MTOR protein may inhibit MTOR protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MTOR protein may degrade MTOR protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MTOR protein may degrade MTOR protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of HARS2 protein may be administered to a subject in need thereof. As a non-limiting example, the inhibitor of the expression or function of HARS2 protein may inhibit HARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of HARS2 protein may inhibit HARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of HARS2 protein may degrade HARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of HARS2 protein may degrade HARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NARS protein may be administered to a subject in need thereof. As a non-limiting example, the inhibitor of the expression or function of NARS protein may inhibit NARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NARS protein may inhibit NARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NARS protein may degrade NARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NARS protein may degrade NARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TSC1 protein may be administered to a subject in need thereof. As a non-limiting example, the inhibitor of the expression or function of TSC1 protein may inhibit TSC1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSC1 protein may inhibit TSC1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSC1 protein may degrade TSC1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSC1 protein may degrade TSC1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POLR3C protein may be administered to a subject in need thereof. As a non-limiting example, the inhibitor of the expression or function of POLR3C protein may inhibit POLR3C protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POLR3C protein may inhibit POLR3C protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POLR3C protein may degrade POLR3C protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POLR3C protein may degrade POLR3C protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of QRSL1 protein may be administered to a subject in need thereof. As a non-limiting example, the inhibitor of the expression or function of QRSL1 protein may inhibit QRSL1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of QRSL1 protein may inhibit QRSL1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of QRSL1 protein may degrade QRSL1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of QRSL1 protein may degrade QRSL1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RPIA protein may be administered to a subject in need thereof. As a non-limiting example, the inhibitor of the expression or function of RPIA protein may inhibit RPIA protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPIA protein may inhibit RPIA protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPIA protein may degrade RPIA protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPIA protein may degrade RPIA protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SDHC protein may be administered to a subject in need thereof. As a non-limiting example, the inhibitor of the expression or function of SDHC protein may inhibit SDHC protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SDHC protein may inhibit SDHC protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SDHC protein may degrade SDHC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SDHC protein may degrade SDHC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX56 protein may be administered to a subject in need thereof. As a non-limiting example, the inhibitor of the expression or function of DDX56 protein may inhibit DDX56 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX56 protein may inhibit DDX56 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX56 protein may degrade DDX56 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX56 protein may degrade DDX56 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EIF4E protein may be administered to a subject in need thereof. As a non-limiting example, the inhibitor of the expression or function of EIF4E protein may inhibit EIF4E protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EIF4E protein may inhibit EIF4E protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EIF4E protein may degrade EIF4E protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EIF4E protein may degrade EIF4E protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX46 protein may be administered to a subject in need thereof. As a non-limiting example, the inhibitor of the expression or function of DDX46 protein may inhibit DDX46 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX46 protein may inhibit DDX46 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX46 protein may degrade DDX46 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX46 protein may degrade DDX46 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IPDH2 protein may be administered to a subject in need thereof. As a non-limiting example, the inhibitor of the expression or function of IMPDH2 protein may inhibit IMPDH2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IMPDH2 protein may inhibit IMPDH2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IMPDH2 protein may degrade IMPDH2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IMPDH2 protein may degrade IMPDH2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SOD2 protein may be administered to a subject in need thereof. As a non-limiting example, the inhibitor of the expression or function of SOD2 protein may inhibit SOD2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SOD2 protein may inhibit SOD2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SOD2 protein may degrade SOD2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SOD2 protein may degrade SOD2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UBE2M protein may be administered to a subject in need thereof. As a non-limiting example, the inhibitor of the expression or function of UBE2M protein may inhibit UBE2M protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UBE2M protein may inhibit UBE2M protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UBE2M protein may degrade UBE2M protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UBE2M protein may degrade UBE2M protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GATC protein may be administered to a subject in need thereof. As a non-limiting example, the inhibitor of the expression or function of GATC protein may inhibit GATC protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GATC protein may inhibit GATC protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GATC protein may degrade GATC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GATC protein may degrade GATC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TSC2 protein may be administered to a subject in need thereof. As a non-limiting example, the inhibitor of the expression or function of TSC2 protein may inhibit TSC2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSC2 protein may inhibit TSC2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSC2 protein may degrade TSC2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSC2 protein may degrade TSC2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PMPCA protein may be administered to a subject in need thereof. As a non-limiting example, the inhibitor of the expression or function of PMPCA protein may inhibit PMPCA protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PMPCA protein may inhibit PMPCA protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PMPCA protein may degrade PMPCA protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PMPCA protein may degrade PMPCA protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TSEN54 protein may be administered to a subject in need thereof. As a non-limiting example, the inhibitor of the expression or function of TSEN54 protein may inhibit TSEN54 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TSEN54 protein may inhibit TSEN54 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TSEN54 protein may degrade TSEN54 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TSEN54 protein may degrade TSEN54 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of FOXM1 protein may be administered to a subject in need thereof. As a non-limiting example, the inhibitor of the expression or function of FOXM1 protein may inhibit FOXM1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of FOXM1 protein may inhibit FOXM1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of FOXM1 protein may degrade FOXM1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of FOXM1 protein may degrade FOXM1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of FARS2 protein may be administered to a subject in need thereof. As a non-limiting example, the inhibitor of the expression or function of FARS2 protein may inhibit FARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of FARS2 protein may inhibit FARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of FARS2 protein may degrade FARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of FARS2 protein may degrade FARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CTPS1 protein may be administered to a subject in need thereof. As a non-limiting example, the inhibitor of the expression or function of CTPS1 protein may inhibit CTPS1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CTPS1 protein may inhibit CTPS1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CTPS1 protein may degrade CTPS1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CTPS1 protein may degrade CTPS1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PARS2 protein may be administered to a subject in need thereof. As a non-limiting example, the inhibitor of the expression or function of PARS2 protein may inhibit PARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PARS2 protein may inhibit PARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PARS2 protein may degrade PARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PARS2 protein may degrade PARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ALG2 protein may be administered to a subject in need thereof. As a non-limiting example, the inhibitor of the expression or function of ALG2 protein may inhibit ALG2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ALG2 protein may inhibit ALG2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ALG2 protein may degrade ALG2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ALG2 protein may degrade ALG2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EIF2B3 protein may be administered to a subject in need thereof. As a non-limiting example, the inhibitor of the expression or function of EIF2B3 protein may inhibit EIF2B3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EIF2B3 protein may inhibit EIF2B3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EIF2B3 protein may degrade EIF2B3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EIF2B3 protein may degrade EIF2B3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CMPK1 protein may be administered to a subject in need thereof. As a non-limiting example, the inhibitor of the expression or function of CMPK1 protein may inhibit CMPK1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CMPK1 protein may inhibit CMPK1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CMPK1 protein may degrade CMPK1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CMPK1 protein may degrade CMPK1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DHDDS protein may be administered to a subject in need thereof. As a non-limiting example, the inhibitor of the expression or function of DHDDS protein may inhibit DHDDS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DHDDS protein may inhibit DHDDS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DHDDS protein may degrade DHDDS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DHDDS protein may degrade DHDDS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SAE1 protein may be administered to a subject in need thereof. As a non-limiting example, the inhibitor of the expression or function of SAE1 protein may inhibit SAE1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SAE1 protein may inhibit SAE1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SAE1 protein may degrade SAE1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SAE1 protein may degrade SAE1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NARS2 protein may be administered to a subject in need thereof. As a non-limiting example, the inhibitor of the expression or function of NARS2 protein may inhibit NARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NARS2 protein may inhibit NARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NARS2 protein may degrade NARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NARS2 protein may degrade NARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PNKP protein may be administered to a subject in need thereof. As a non-limiting example, the inhibitor of the expression or function of PNKP protein may inhibit PNKP protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PNKP protein may inhibit PNKP protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PNKP protein may degrade PNKP protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PNKP protein may degrade PNKP protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PDSS1 protein may be administered to a subject in need thereof. As a non-limiting example, the inhibitor of the expression or function of PDSS1 protein may inhibit PDSS1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PDSS1 protein may inhibit PDSS1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PDSS1 protein may degrade PDSS1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PDSS1 protein may degrade PDSS1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POLR3K protein may be administered to a subject in need thereof. As a non-limiting example, the inhibitor of the expression or function of POLR3K protein may inhibit POLR3K protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POLR3K protein may inhibit POLR3K protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POLR3K protein may degrade POLR3K protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POLR3K protein may degrade POLR3K protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of AHCY protein may be administered to a subject in need thereof. As a non-limiting example, the inhibitor of the expression or function of AHCY protein may inhibit AHCY protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of AHCY protein may inhibit AHCY protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of AHCY protein may degrade AHCY protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of AHCY protein may degrade AHCY protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NAE1 protein may be administered to a subject in need thereof. As a non-limiting example, the inhibitor of the expression or function of NAE1 protein may inhibit NAE1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NAE1 protein may inhibit NAE1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NAE1 protein may degrade NAE1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NAE1 protein may degrade NAE1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UBIAD1 protein may be administered to a subject in need thereof. As a non-limiting example, the inhibitor of the expression or function of UBIAD1 protein may inhibit UBIAD1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UBIAD1 protein may inhibit UBIAD1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UBIAD1 protein may degrade UBIAD1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UBIAD1 protein may degrade UBIAD1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RPUSD4 protein may be administered to a subject in need thereof. As a non-limiting example, the inhibitor of the expression or function of RPUSD4 protein may inhibit RPUSD4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RPUSD4 protein may inhibit RPUSD4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RPUSD4 protein may degrade RPUSD4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RPUSD4 protein may degrade RPUSD4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EARS2 protein may be administered to a subject in need thereof. As a non-limiting example, the inhibitor of the expression or function of EARS2 protein may inhibit EARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EARS2 protein may inhibit EARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EARS2 protein may degrade EARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EARS2 protein may degrade EARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GMPPB protein may be administered to a subject in need thereof. As a non-limiting example, the inhibitor of the expression or function of GMPPB protein may inhibit GMPPB protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GMPPB protein may inhibit GMPPB protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GMPPB protein may degrade GMPPB protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GMPPB protein may degrade GMPPB protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of LIAS protein may be administered to a subject in need thereof. As a non-limiting example, the inhibitor of the expression or function of LIAS protein may inhibit LIAS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of LIAS protein may inhibit LIAS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of LIAS protein may degrade LIAS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of LIAS protein may degrade LIAS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PPP4C protein may be administered to a subject in need thereof. As a non-limiting example, the inhibitor of the expression or function of PPP4C protein may inhibit PPP4C protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PPP4C protein may inhibit PPP4C protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PPP4C protein may degrade PPP4C protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PPP4C protein may degrade PPP4C protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NSUN4 protein may be administered to a subject in need thereof. As a non-limiting example, the inhibitor of the expression or function of NSUN4 protein may inhibit NSUN4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NSUN4 protein may inhibit NSUN4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NSUN4 protein may degrade NSUN4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NSUN4 protein may degrade NSUN4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DLD protein may be administered to a subject in need thereof. As a non-limiting example, the inhibitor of the expression or function of DLD protein may inhibit DLD protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DLD protein may inhibit DLD protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DLD protein may degrade DLD protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DLD protein may degrade DLD protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TRMT5 protein may be administered to a subject in need thereof. As a non-limiting example, the inhibitor of the expression or function of TRMT5 protein may inhibit TRMT5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TRMT5 protein may inhibit TRMT5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TRMT5 protein may degrade TRMT5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TRMT5 protein may degrade TRMT5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of AASDHPPT protein may be administered to a subject in need thereof. As a non-limiting example, the inhibitor of the expression or function of AASDHPPT protein may inhibit AASDHPPT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of AASDHPPT protein may inhibit AASDHPPT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of AASDHPPT protein may degrade AASDHPPT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of AASDHPPT protein may degrade AASDHPPT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EIF5A protein may be administered to a subject in need thereof. As a non-limiting example, the inhibitor of the expression or function of EIF5A protein may inhibit EIF5A protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EIF5A protein may inhibit EIF5A protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EIF5A protein may degrade EIF5A protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EIF5A protein may degrade EIF5A protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POT1 protein may be administered to a subject in need thereof. As a non-limiting example, the inhibitor of the expression or function of POT1 protein may inhibit POT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POT1 protein may inhibit POT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POT1 protein may degrade POT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POT1 protein may degrade POT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DHX9 protein may be administered to a subject in need thereof. As a non-limiting example, the inhibitor of the expression or function of DHX9 protein may inhibit DHX9 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DHX9 protein may inhibit DHX9 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DHX9 protein may degrade DHX9 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DHX9 protein may degrade DHX9 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of LONP1 protein may be administered to a subject in need thereof. As a non-limiting example, the inhibitor of the expression or function of LONP1 protein may inhibit LONP1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of LONP1 protein may inhibit LONP1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of LONP1 protein may degrade LONP1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of LONP1 protein may degrade LONP1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PPP6C protein may be administered to a subject in need thereof. As a non-limiting example, the inhibitor of the expression or function of PPP6C protein may inhibit PPP6C protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PPP6C protein may inhibit PPP6C protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PPP6C protein may degrade PPP6C protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PPP6C protein may degrade PPP6C protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SKIV2L2 protein may be administered to a subject in need thereof. As a non-limiting example, the inhibitor of the expression or function of SKIV2L2 protein may inhibit SKIV2L2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SKIV2L2 protein may inhibit SKIV2L2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SKIV2L2 protein may degrade SKIV2L2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SKIV2L2 protein may degrade SKIV2L2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PTDSS1 protein may be administered to a subject in need thereof. As a non-limiting example, the inhibitor of the expression or function of PTDSS1 protein may inhibit PTDSS1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PTDSS1 protein may inhibit PTDSS1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PTDSS1 protein may degrade PTDSS1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PTDSS1 protein may degrade PTDSS1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of USP5 protein may be administered to a subject in need thereof. As a non-limiting example, the inhibitor of the expression or function of USP5 protein may inhibit USP5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of USP5 protein may inhibit USP5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of USP5 protein may degrade USP5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of USP5 protein may degrade USP5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of VPS52 protein may be administered to a subject in need thereof. As a non-limiting example, the inhibitor of the expression or function of VPS52 protein may inhibit VPS52 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of VPS52 protein may inhibit VPS52 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of VPS52 protein may degrade VPS52 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of VPS52 protein may degrade VPS52 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TKT protein may be administered to a subject in need thereof. As a non-limiting example, the inhibitor of the expression or function of TKT protein may inhibit TKT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TKT protein may inhibit TKT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TKT protein may degrade TKT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TKT protein may degrade TKT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TRMT61A protein may be administered to a subject in need thereof. As a non-limiting example, the inhibitor of the expression or function of TRMT61A protein may inhibit TRMT61A protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TRMT61A protein may inhibit TRMT61A protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TRMT61A protein may degrade TRMT61A protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TRMT61A protein may degrade TRMT61A protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of N6AMT1 protein may be administered to a subject in need thereof. As a non-limiting example, the inhibitor of the expression or function of N6AMT1 protein may inhibit N6AMT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of N6AMT1 protein may inhibit N6AMT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of N6AMT1 protein may degrade N6AMT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of N6AMT1 protein may degrade N6AMT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GGPS1 protein may be administered to a subject in need thereof. As a non-limiting example, the inhibitor of the expression or function of GGPS1 protein may inhibit GGPS1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GGPS1 protein may inhibit GGPS1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GGPS1 protein may degrade GGPS1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GGPS1 protein may degrade GGPS1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EFTUD1 protein may be administered to a subject in need thereof. As a non-limiting example, the inhibitor of the expression or function of EFTUD1 protein may inhibit EFTUD1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EFTUD1 protein may inhibit EFTUD1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EFTUD1 protein may degrade EFTUD1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EFTUD1 protein may degrade EFTUD1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ACAD9 protein may be administered to a subject in need thereof. As a non-limiting example, the inhibitor of the expression or function of ACAD9 protein may inhibit ACAD9 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ACAD9 protein may inhibit ACAD9 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ACAD9 protein may degrade ACAD9 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ACAD9 protein may degrade ACAD9 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SETD1A protein may be administered to a subject in need thereof. As a non-limiting example, the inhibitor of the expression or function of SETD1A protein may inhibit SETD1A protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SETD1A protein may inhibit SETD1A protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SETD1A protein may degrade SETD1A protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SETD1A protein may degrade SETD1A protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IPO 11 protein may be administered to a subject in need thereof. As a non-limiting example, the inhibitor of the expression or function of IPO11 protein may inhibit IPO11 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IPO11 protein may inhibit IPO11 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IPO11 protein may degrade IPO11 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IPO11 protein may degrade IPO11 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EIF3I protein may be administered to a subject in need thereof. As a non-limiting example, the inhibitor of the expression or function of EIF3I protein may inhibit EIF3I protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EIF3I protein may inhibit EIF3I protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EIF3I protein may degrade EIF3I protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EIF3I protein may degrade EIF3I protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of METTL16 protein may be administered to a subject in need thereof. As a non-limiting example, the inhibitor of the expression or function of METTL16 protein may inhibit METTL16 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of METTL16 protein may inhibit METTL16 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of METTL16 protein may degrade METTL16 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of METTL16 protein may degrade METTL16 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MASTL protein may be administered to a subject in need thereof. As a non-limiting example, the inhibitor of the expression or function of MASTL protein may inhibit MASTL protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MASTL protein may inhibit MASTL protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MASTL protein may degrade MASTL protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MASTL protein may degrade MASTL protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX51 protein may be administered to a subject in need thereof. As a non-limiting example, the inhibitor of the expression or function of DDX51 protein may inhibit DDX51 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX51 protein may inhibit DDX51 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX51 protein may degrade DDX51 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX51 protein may degrade DDX51 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ADAT3 protein may be administered to a subject in need thereof. As a non-limiting example, the inhibitor of the expression or function of ADAT3 protein may inhibit ADAT3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ADAT3 protein may inhibit ADAT3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ADAT3 protein may degrade ADAT3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ADAT3 protein may degrade ADAT3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ZNRD1 protein may be administered to a subject in need thereof. As a non-limiting example, the inhibitor of the expression or function of ZNRD1 protein may inhibit ZNRD1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ZNRD1 protein may inhibit ZNRD1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ZNRD1 protein may degrade ZNRD1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ZNRD1 protein may degrade ZNRD1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of OGT protein may be administered to a subject in need thereof. As a non-limiting example, the inhibitor of the expression or function of OGT protein may inhibit OGT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of OGT protein may inhibit OGT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of OGT protein may degrade OGT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of OGT protein may degrade OGT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IDI1 protein may be administered to a subject in need thereof. As a non-limiting example, the inhibitor of the expression or function of IDI1 protein may inhibit IDI1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IDI1 protein may inhibit IDI1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IDI1 protein may degrade IDI1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IDI1 protein may degrade IDI1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IMP4 protein may be administered to a subject in need thereof. As a non-limiting example, the inhibitor of the expression or function of IMP4 protein may inhibit IMP4 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IMP4 protein may inhibit IMP4 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IMP4 protein may degrade IMP4 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IMP4 protein may degrade IMP4 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of FTSJ3 protein may be administered to a subject in need thereof. As a non-limiting example, the inhibitor of the expression or function of FTSJ3 protein may inhibit FTSJ3 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of FTSJ3 protein may inhibit FTSJ3 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of FTSJ3 protein may degrade FTSJ3 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of FTSJ3 protein may degrade FTSJ3 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EXOSC8 protein may be administered to a subject in need thereof. As a non-limiting example, the inhibitor of the expression or function of EXOSC8 protein may inhibit EXOSC8 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EXOSC8 protein may inhibit EXOSC8 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EXOSC8 protein may degrade EXOSC8 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EXOSC8 protein may degrade EXOSC8 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GSG2 protein may be administered to a subject in need thereof. As a non-limiting example, the inhibitor of the expression or function of GSG2 protein may inhibit GSG2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GSG2 protein may inhibit GSG2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GSG2 protein may degrade GSG2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GSG2 protein may degrade GSG2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PI4KA protein may be administered to a subject in need thereof. As a non-limiting example, the inhibitor of the expression or function of PI4KA protein may inhibit PI4KA protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PI4KA protein may inhibit PI4KA protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PI4KA protein may degrade PI4KA protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PI4KA protein may degrade PI4KA protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of NSMCE2 protein may be administered to a subject in need thereof. As a non-limiting example, the inhibitor of the expression or function of NSMCE2 protein may inhibit NSMCE2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of NSMCE2 protein may inhibit NSMCE2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of NSMCE2 protein may degrade NSMCE2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of NSMCE2 protein may degrade NSMCE2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDX52 protein may be administered to a subject in need thereof. As a non-limiting example, the inhibitor of the expression or function of DDX52 protein may inhibit DDX52 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDX52 protein may inhibit DDX52 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDX52 protein may degrade DDX52 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDX52 protein may degrade DDX52 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DDOST protein may be administered to a subject in need thereof. As a non-limiting example, the inhibitor of the expression or function of DDOST protein may inhibit DDOST protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DDOST protein may inhibit DDOST protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DDOST protein may degrade DDOST protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DDOST protein may degrade DDOST protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CSNK2B protein may be administered to a subject in need thereof. As a non-limiting example, the inhibitor of the expression or function of CSNK2B protein may inhibit CSNK2B protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CSNK2B protein may inhibit CSNK2B protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CSNK2B protein may degrade CSNK2B protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CSNK2B protein may degrade CSNK2B protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UBA2 protein may be administered to a subject in need thereof. As a non-limiting example, the inhibitor of the expression or function of UBA2 protein may inhibit UBA2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UBA2 protein may inhibit UBA2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UBA2 protein may degrade UBA2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UBA2 protein may degrade UBA2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RABGGTA protein may be administered to a subject in need thereof. As a non-limiting example, the inhibitor of the expression or function of RABGGTA protein may inhibit RABGGTA protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RABGGTA protein may inhibit RABGGTA protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RABGGTA protein may degrade RABGGTA protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RABGGTA protein may degrade RABGGTA protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SOD1 protein may be administered to a subject in need thereof. As a non-limiting example, the inhibitor of the expression or function of SOD1 protein may inhibit SOD1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SOD1 protein may inhibit SOD1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SOD1 protein may degrade SOD1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SOD1 protein may degrade SOD1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TRIT1 protein may be administered to a subject in need thereof. As a non-limiting example, the inhibitor of the expression or function of TRIT1 protein may inhibit TRIT1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TRIT1 protein may inhibit TRIT1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TRIT1 protein may degrade TRIT1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TRIT1 protein may degrade TRIT1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TYMS protein may be administered to a subject in need thereof. As a non-limiting example, the inhibitor of the expression or function of TYMS protein may inhibit TYMS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TYMS protein may inhibit TYMS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TYMS protein may degrade TYMS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TYMS protein may degrade TYMS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RNF168 protein may be administered to a subject in need thereof. As a non-limiting example, the inhibitor of the expression or function of RNF168 protein may inhibit RNF168 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RNF168 protein may inhibit RNF168 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RNF168 protein may degrade RNF168 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RNF168 protein may degrade RNF168 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of UBE2I protein may be administered to a subject in need thereof. As a non-limiting example, the inhibitor of the expression or function of UBE2I protein may inhibit UBE2I protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of UBE2I protein may inhibit UBE2I protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of UBE2I protein may degrade UBE2I protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of UBE2I protein may degrade UBE2I protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GARS protein may be administered to a subject in need thereof. As a non-limiting example, the inhibitor of the expression or function of GARS protein may inhibit GARS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GARS protein may inhibit GARS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GARS protein may degrade GARS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GARS protein may degrade GARS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of IPO13 protein may be administered to a subject in need thereof. As a non-limiting example, the inhibitor of the expression or function of IPO13 protein may inhibit IPO13 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of IPO13 protein may inhibit IPO13 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of IPO13 protein may degrade IPO13 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of IPO13 protein may degrade IPO13 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SMARCB1 protein may be administered to a subject in need thereof. As a non-limiting example, the inhibitor of the expression or function of SMARCB1 protein may inhibit SMARCB1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SMARCB1 protein may inhibit SMARCB1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SMARCB1 protein may degrade SMARCB1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SMARCB1 protein may degrade SMARCB1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of EIF2B1 protein may be administered to a subject in need thereof. As a non-limiting example, the inhibitor of the expression or function of EIF2B1 protein may inhibit EIF2B1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of EIF2B1 protein may inhibit EIF2B1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of EIF2B1 protein may degrade EIF2B1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of EIF2B1 protein may degrade EIF2B1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of RNASEH1 protein may be administered to a subject in need thereof. As a non-limiting example, the inhibitor of the expression or function of RNASEH1 protein may inhibit RNASEH1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of RNASEH1 protein may inhibit RNASEH1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of RNASEH1 protein may degrade RNASEH1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of RNASEH1 protein may degrade RNASEH1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of MCAT protein may be administered to a subject in need thereof. As a non-limiting example, the inhibitor of the expression or function of MCAT protein may inhibit MCAT protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of MCAT protein may inhibit MCAT protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of MCAT protein may degrade MCAT protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of MCAT protein may degrade MCAT protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of XRN2 protein may be administered to a subject in need thereof. As a non-limiting example, the inhibitor of the expression or function of XRN2 protein may inhibit XRN2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of XRN2 protein may inhibit XRN2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of XRN2 protein may degrade XRN2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of XRN2 protein may degrade XRN2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of POP5 protein may be administered to a subject in need thereof. As a non-limiting example, the inhibitor of the expression or function of POP5 protein may inhibit POP5 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of POP5 protein may inhibit POP5 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of POP5 protein may degrade POP5 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of POP5 protein may degrade POP5 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of CS protein may be administered to a subject in need thereof. As a non-limiting example, the inhibitor of the expression or function of CS protein may inhibit CS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of CS protein may inhibit CS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of CS protein may degrade CS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of CS protein may degrade CS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of FNTB protein may be administered to a subject in need thereof. As a non-limiting example, the inhibitor of the expression or function of FNTB protein may inhibit FNTB protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of FNTB protein may inhibit FNTB protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of FNTB protein may degrade FNTB protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of FNTB protein may degrade FNTB protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of DARS2 protein may be administered to a subject in need thereof. As a non-limiting example, the inhibitor of the expression or function of DARS2 protein may inhibit DARS2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of DARS2 protein may inhibit DARS2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of DARS2 protein may degrade DARS2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of DARS2 protein may degrade DARS2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of TFRC protein may be administered to a subject in need thereof. As a non-limiting example, the inhibitor of the expression or function of TFRC protein may inhibit TFRC protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of TFRC protein may inhibit TFRC protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of TFRC protein may degrade TFRC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of TFRC protein may degrade TFRC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SLC7A6OS protein may be administered to a subject in need thereof. As a non-limiting example, the inhibitor of the expression or function of SLC7A6OS protein may inhibit SLC7A6OS protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SLC7A6OS protein may inhibit SLC7A6OS protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SLC7A6OS protein may degrade SLC7A6OS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SLC7A6OS protein may degrade SLC7A6OS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GNB2L1 protein may be administered to a subject in need thereof. As a non-limiting example, the inhibitor of the expression or function of GNB2L1 protein may inhibit GNB2L1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GNB2L1 protein may inhibit GNB2L 1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GNB2L1 protein may degrade GNB2L1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GNB2L1 protein may degrade GNB2L1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of GFER protein may be administered to a subject in need thereof. As a non-limiting example, the inhibitor of the expression or function of GFER protein may inhibit GFER protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of GFER protein may inhibit GFER protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of GFER protein may degrade GFER protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of GFER protein may degrade GFER protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of ATP6AP2 protein may be administered to a subject in need thereof. As a non-limiting example, the inhibitor of the expression or function of ATP6AP2 protein may inhibit ATP6AP2 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of ATP6AP2 protein may inhibit ATP6AP2 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of ATP6AP2 protein may degrade ATP6AP2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of ATP6AP2 protein may degrade ATP6AP2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of SLC25A19 protein may be administered to a subject in need thereof. As a non-limiting example, the inhibitor of the expression or function of SLC25A19 protein may inhibit SLC25A19 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of SLC25A19 protein may inhibit SLC25A19 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of SLC25A19 protein may degrade SLC25A19 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of SLC25A19 protein may degrade SLC25A19 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, an effective amount of an SHP2 inhibitor described herein and an effective amount of an inhibitor of the expression or function and/or a degrader of PEAR1 protein may be administered to a subject in need thereof. As a non-limiting example, the inhibitor of the expression or function of PEAR1 protein may inhibit PEAR1 protein expression or function by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of the expression or function of PEAR1 protein may inhibit PEAR1 protein expression or function by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the degrader of PEAR1 protein may degrade PEAR1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the degrader of PEAR1 protein may degrade PEAR1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the method comprises administering to the subject an effective amount of an SHP2 inhibitor and an inhibitor of the function of one or more proteins selected from TFRC, SLC7A6OS, GNB2L1, GFER, ATP6AP2, SLC25A19, and/or PEAR1.

In various embodiments, the inhibitor of activity may inhibit the activity of TFRC protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of TFRC protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of SLC7A6OS protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of SLC7A6OS protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of GNB2L1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of GNB2L1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of GFER protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of GFER protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of ATP6AP2 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of ATP6AP2 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of SLC25A19 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of SLC25A19 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the inhibitor of activity may inhibit the activity of PEAR1 protein by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. In some embodiments, the inhibitor of activity may inhibit the activity of PEAR1 protein by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In various embodiments, the SHP2 inhibitor and the inhibitor of the expression or function or degrader of the one or more proteins described herein are administered to the subject simultaneously. In some embodiments, the SHP2 inhibitor and the inhibitor of the expression or function or degrader of the one or more proteins described herein are administered to the subject simultaneously in one composition. In some embodiments, the SHP2 inhibitor and the inhibitor of the expression or function or degrader of the one or more proteins described herein are administered to the subject simultaneously in different compositions. In various embodiments, the SHP2 inhibitor and the inhibitor of the expression or function or degrader of the one or more proteins described herein are administered to the subject sequentially.

In some embodiments, when the inhibitor of the expression or function of the one or more proteins or the degrader of the one or more proteins described herein and the SHP2 inhibitor are administered to the subject sequentially (e.g., in different compositions), the inhibitor of the expression or function of the one or more proteins or the degrader of the one or more proteins may be administered as a first component of a dosing regimen and the SHP2 inhibitor may be administered as a second component of a dosing regimen (i.e., the inhibitor of the expression or function of the one or more proteins or the degrader of the one or more proteins may be administered before the SHP2 inhibitor).

In some embodiments, when the inhibitor of the expression or function of the one or more proteins or the degrader of the one or more proteins described herein and the SHP2 inhibitor are administered to the subject sequentially (e.g., in different compositions), the SHP2 inhibitor may be administered as a first component of a dosing regimen and the inhibitor of the expression or function of the one or more proteins or the degrader of the one or more proteins may be administered as a second component of a dosing regimen (i.e., the SHP2 inhibitor may be administered before the inhibitor of expression or function of the one or more proteins or the degrader of the one or more proteins).

In some embodiments, the SHP2 inhibitor and/or the inhibitor of the expression or function or degrader of the one or more proteins described herein can be administered to the subject by way of any route of administration of the present disclosure. As a non-limiting example, the SHP2 inhibitor and/or the inhibitor of the expression or function or degrader of the one or more proteins described herein can be administered orally or intravenously.

In some aspects, provided herein is a method of treating a KRAS mutant cancer in a subject in need thereof, comprising administering to the subject an effective amount of a SHP2 inhibitor and a binding partner, wherein said binding partner specifically binds to one or more proteins selected from TFRC, SLC7A6OS, GNB2L1, GFER, ATP6AP2, SLC25A19, and PEAR1. In some embodiments, the SHP2 inhibitor and the binding partner of said one or more proteins are administered simultaneously. In some embodiments, the SHP2 inhibitor and the binding partner of said one or more proteins are administered simultaneously in one composition. In some embodiments, the SHP2 inhibitor and the binding partner of said one or more proteins are administered simultaneously in different compositions. In some embodiments, the SHP2 inhibitor and the binding partner of said one or more proteins are administered sequentially. In some embodiments, the SHP2 inhibitor and/or the binding partner of said one or more proteins is administered orally or intravenously.

In some embodiments, the binding partner of said one or more proteins comprises an intact antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a single-chain variable fragment (scFv), a Bi-specific T-cell engager (BiTE), a bispecific killer cell engager (BiKE), a CrossMab, a tri-specific binding partner, or a chimeric antigen receptor (CAR). In certain embodiments, the binding partner of said one or more proteins is conjugated to a detectable label, or a chemotherapeutic agent, a radioisotope, or a toxin. In certain embodiments, the binding partner of said one or more proteins is a component of a fusion protein. In certain embodiments, the binding partner of said one or more proteins comprises a chimeric antigen receptor (CAR). In certain embodiments, the binding partner of said one or more proteins is expressed by a T cell or a natural killer cell.

In some embodiments, a KRAS mutant cancer cell described herein may comprise any number of various mutations in KRAS described herein, or combinations thereof. For instance, without limitation, a KRAS mutant cancer cell may comprise a mutation such as, but not limited to a KRAS G12 mutation, a KRAS G13 mutation, a KRAS H61 mutation, or a KRAS K117 mutation. In some embodiments, when the KRAS mutant cancer cell described herein comprises a KRAS G12 mutation, the KRAS G12 mutation may be, e.g., a G12C mutation, a G12V mutation, or a G12D mutation.

In some embodiments, the KRAS mutant cancer cell described herein comprises a KRAS G12C mutation.

In various embodiments, the KRAS mutant cancer cell described herein comprises a KRAS G13D mutation. In various embodiments, the KRAS mutant cancer cell comprises a KRAS H61 mutation. Non-limiting examples of a KRAS H61 mutation include a Q61H mutation, a Q61L mutation, and a Q61R mutation.

In some embodiments, the KRAS mutant cancer cell described herein comprises a KRAS K117N mutation.

In some embodiments, a KRAS mutant cancer cell comprising any of the above described mutations in KRAS may, in addition, also comprise a mutation, e.g., in STK11 (also called Liver kinase B1 [LKB1]) gene and/or Kelch Like ECH Associated Protein 1 (KEAP1) gene.

As discussed above, KRAS mutant cancer cells can comprise any KRAS mutant cell type known to those of skill in the art such as, but not limited to, any of various KRAS mutant cancer cell described herein.

In some embodiments, a KRAS mutant cancer cell described herein may be derived from a KRAS mutant cancer. In certain embodiments, the KRAS mutant cancer is lung cancer, colorectal cancer, or pancreatic cancer. In some embodiments, the lung cancer is non-small cell lung cancer.

In some embodiments, the cancer is a glioma cancer. In certain embodiments, the cancer is ovarian cancer. In certain embodiments, the cancer is a lung cancer. In some embodiments, the cancer is non-small cell lung cancer. In some embodiments, the cancer is a head and neck cancer. In some embodiments, the cancer is a colorectal cancer. In some embodiments, the cancer is a stomach cancer. In some embodiments, the cancer is a renal cancer. In some embodiments, the cancer is adult renal cell carcinoma or pediatric renal cell carcinoma. In some embodiments, the cancer is a skin cancer. In some embodiments, the cancer is a cervical cancer. In some embodiments, the cancer is brain cancer. In some embodiments, the cancer is breast cancer. In some embodiments, the cancer is triple negative breast cancer. In some embodiments, the cancer is a prostate cancer. In further embodiments, the cancer is a bladder cancer.

In certain embodiments the cancer is a hematologic malignancy (e.g., leukemia, a lymphoma, or a myeloma). Leukemia includes, but is not limited to, acute myeloid leukemia (AML), chronic lymphocytic leukemia (CLL), acute lymphocytic leukemia (ALL), liver acute lymphoblastic leukemia, and chronic myeloid leukemia (CMIL). Non-limiting examples of lymphoma are non-Hodgkin's lymphoma or Hodgkin's lymphoma. In some embodiments, the lymphoma is anaplastic large cell lymphoma (ALCL). In further embodiments, the non-Hodgkin's lymphoma is Diffuse Large B-cell Lymphoma (DLBCL).

In some embodiments, the cancer is neuroblastoma, inflammatory myofibroblastic tumor, colonic adeno-carcinoma, glioblastoma, glioblastoma multiforme, anaplastic thyroid cancer, cholangiocarcinoma, angiosarcoma, epithelioid hemangioendothelioma, intrahepatic cholangiocarcinoma, thyroid cancer, spitzoid neoplasms, sarcomas, astrocytoma, brain lower grade glioma, secretory breast carcinoma, mammary analogue carcinoma, congenital mesoblastic nephroma, congenital fibrosarcomas, Ph-like acute lymphoblastic leukemia, thyroid carcinoma, head and neck squamous cell carcinoma, pediatric glioma CML, lung squamous carcinoma, ovarian serous cystadenocarcinoma, skin cutaneous melanoma, castrate-resistant prostate cancer, serous and clear cell endometrial cancer, oral cancer, endometrial cancer, endocrine cancer, gastric cancer, esophageal cancer, laryngeal cancer, colon cancer, bone cancer, testicular cancer, rectal cancer, kidney cancer, liver cancer, stomach cancer, metastatic non-small cell lung cancer, colorectal cancer, metastatic colorectal cancer, pancreatic cancer, metastatic pancreatic cancer, metastatic uterine cancer. In some embodiments, the cancer is adenocarcinomas, adenomatoid tumors, alveolar (bronchiolar) carcinoma, ampullary carcinoma, angioma, basal cell carcinoma, benign chondroma, botryoid sarcoma (embryonal rhabdomyosarcoma), bronchial adenoma, bronchogenic carcinoma undifferentiated large cell, bronchogenic carcinoma undifferentiated small cell, bronchogenic carcinoma, carcinoid tumors, carcinomas, cervical carcinoma, chondroblastoma, chondromatous hamartoma, chondromyxofibroma, chondrosarcoma, choriocarcinoma, clear cell carcinoma, congenital tumors, dermatofibroma, ductal adenocarcinoma, dysgerminoma, embryonal carcinoma, endometrial carcinoma, ependymoma, esophageal squamous cell carcinoma, Ewing's sarcoma, fallopian tubes cancer, fibroadenoma, fibromas, gall bladder carcinoma, gastrinoma, germinoma (pinealoma), gliomas, gliomatosis, glucagonoma, granuloma, granulosa-thecal cell tumors, hamartoma, hemangiomas, hepatoblastoma, hepatocellular adenoma, hepatoma (hepatocellular carcinoma), insulinoma, interstitial cell carcinoma, intraepithelial carcinoma, Kaposi's sarcoma, keloids, large bowel cancers, leiomyomas, leiomyosarcomas, lipomas, liposarcoma, malignant fibrous histiocytoma, malignant giant cell tumor chordoma, malignant lymphoma (reticulum cell sarcoma), malignant melanoma, malignant teratoma, medulloblastoma, melanoma, meningio sarcoma, meningioma, mesothelioma, moles dysplastic nevi, mucinous cystadenocarcinoma, multiple myeloma, myelodysplastic syndrome, myeloproliferative diseases, myxoma, neurofibroma, oligodendroglioma, osteitis deformans, osteochronfroma (osteocartilaginous exostoses), osteogenic sarcoma (osteosarcoma), osteoid osteoma and giant cell tumors, osteoma, ovarian carcinoma, pre-tumor cervical dysplasia, prostate sarcoma, retinoblastoma, rhabdomyoma, Rhabdomyosarcoma, schwannoma, seminoma, Sertoli-Leydig cell tumors, small bowel cancers, spinal cord neurofibroma, squamous cell carcinomas, teratocarcinoma, teratomas, testis cancers, transitional cell carcinomas, tubular adenoma, unclassified carcinomas, urethral cancers, vaginal cancers, villous adenoma, vipoma, vulvar cancers, Wilm's tumor (nephroblastoma), and xanthoma.

In some embodiments, the KRAS mutant cancer is uterine cancer or gastric cancer.

In some embodiments, the KRAS mutant cancer described herein may be resistant to a treatment with an SHP2 inhibitor (e.g., any of various SHP2 inhibitors described herein) when the SHP2 inhibitor is administered in the absence of the inhibitor of expression or function or degrader of the one or more proteins described herein. Non-limiting examples of SHP2 inhibitors include BBP-398, TNO155, RMC-4630, JAB-3068, RLY-1971, and ERAS-601, pharmaceutically acceptable salts thereof, and any combinations thereof.

In some embodiments, the subject is human. In some embodiments, the subject is a veterinary animal (e.g., cats, dogs, cows, horses, sheep, pigs, etc.) or an experimental animal model.

### Other Non-limiting Examples of Inhibitors Protein Expression

In some embodiments, an inhibitor of protein expression that may be used in the practice of the present disclosure can be an siRNA, an shRNA, an antisense oligonucleotide, a miRNA, or a site-specific nuclease. Non-limiting examples of such inhibitors of protein expression are described below.

In some embodiments, the inhibitor is a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e., a ssRNA molecule comprising just an antisense strand) or double stranded (i.e., a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 more nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprise an antisense strand having a nucleotide sequence that has up 0, 1, 2, or 3 mismatches over 15-22 bases with a target sequence. In some embodiments, siRNA molecules comprise an antisense strand having a nucleotide sequence that has 0, 1, or 2 mismatches over 15-22 bases with a target sequence. In some embodiments, siRNA molecules comprise an antisense strand having a nucleotide sequence that has 0 or 1 mismatch over 15-22 bases with a target sequence. In some embodiments, siRNA molecules comprise an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated in the present disclosure. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In some embodiments, the inhibitor is a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

In some embodiments, an inhibitor of protein expression comprises a site-specific nuclease. In some embodiments, the site-specific nuclease comprises a DNA nuclease such as an engineered (e.g., programmable or targetable) DNA nuclease to induce genome editing of a target DNA sequence. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof.

In some embodiments, an inhibitor of protein expression is an antisense oligonucleotide (ASO). An ASO can downregulate a target, for example, by steric hindrance of ribosomal activity, by causing RNase H endonuclease cleavage of a target RNA, or by altering splicing or by inhibiting 5′cap formation. An ASO can generally comprise a short nucleotide sequence which is substantially complementary to a target nucleotide sequence in a pre-mRNA molecule, an mRNA molecule, or a heterogeneous nuclear RNA (hnRNA). The degree of complementarity (or substantial complementarity) of the antisense sequence can be such that a molecule comprising the antisense sequence may form a stable double-stranded hybrid with the target nucleotide sequence in the RNA molecule. Without wishing to be bound by theory, “complementarity” of nucleic acids can mean that a nucleotide sequence in one strand of nucleic acid, e.g., due to orientation of its nucleobase groups, forms hydrogen bonds with another sequence on an opposing nucleic acid strand. The complementary bases in DNA are generally A paired with T and C paired with G. In RNA, the complementary bases are generally C with paired with G and U paired with A. Complementarity can be perfect or substantial/sufficient. Perfect complementarity between two nucleic acids means that the two nucleic acids can form a duplex in which every base within the duplex is bonded to a complementary base by Watson-Crick pairing. “Substantial” or “sufficient” complementarity means that a sequence in one strand is not completely and/or perfectly complementary to a sequence in an opposing strand but that sufficient bonding takes place between bases on the two strands to form a stable hybrid complex in set of hybridization conditions (e.g., temperature and/or salt concentration). Such conditions can be determined by, e.g., empirical determination of Tₘ (melting temperature) by employing routine methods in the art or by using the sequences and standard mathematical calculations to predict the Tₘ of hybridized strands. Tₘ can include the temperature at which a population of hybridization complexes formed between two nucleic acid strands are 50% denatured (i.e., a population of double-stranded nucleic acid molecules becomes half dissociated into single strands). At a temperature below the Tₘ, formation of a hybridization complex can be favored, while at a temperature above the Tₘ, melting or separation of the strands in the hybridization complex can be favored.

In some embodiments, an ASO is a morpholino or a gapmer.

Antisense oligonucleotides can be synthetic and chemically modified.

In some embodiments, antisense oligonucleotides may be 100% complementary to the target sequence, or may comprise mismatches. so long as a heteroduplex formed between the oligonucleotide and the target sequence is sufficiently stable to tolerate the action of modes of degradation which may occur in vivo, e.g., by way of cellular nucleases. Mismatches, if present, are generally less destabilizing toward the end regions of the hybrid duplex than in the middle. The number of mismatches permitted can depend on, e.g., the percentage of G:C base pairs in the duplex, the length of the oligonucleotide, and/or the position of the mismatch(es) in the duplex, according to principles of duplex stability within the knowledge of one skilled in the art. In some embodiments, an oligonucleotide may have about 70% to about 100% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

### Non-Limiting Examples of Protein Degraders

In some embodiments, degrading a protein described herein can comprise administering to a cell or a subject of the present disclosure an effective amount of, e.g., without limitation, a proteolysis targeting chimera (PROTAC), an autophagy-targeting chimera (AUTAC), a lysosome-targeting chimera (LYTAC), or a molecular glue.

Proteolysis targeting chimeras (PROTACs) are bifunctional molecules that combine a ligand for an E3 ligase with a second ligand that targets a protein of interest and catalyze its polyubiquitination and eventual proteasomal degradation. Both ends of the PROTACs are connected via a linker. First generation PROTACs used peptides to recruit a protein of interest to E3 ligases, but subsequent ones have relied on smaller and more cell-permeable synthetic ligands. These include hydroxyproline derivatives and molecules derived from thalidomide, which bind the von Hippel-Lindau protein (VHL) and cereblon (CRBN), respectively. VHL and CRBN are the substrate receptors of two cullin-RING ubiquitin ligase (CRL) complexes, namely CRL2^{VHL} and CRL4^{CRBN}.

A non-limiting description of PROTACs is available in doi: 10.1021/acscentsci.0c00411, incorporated herein by reference in its entirety and for all purposes as if fully set forth herein. In some embodiments, the PROTAC can be LC-1 or LC-2.

In some embodiments, a protein degrader of the present disclosure may comprise an autophagy-targeting chimera (AUTAC), e.g., as described in doi. org/10.1080/15548627.2020.1718362, incorporated herein by reference in its entirety and for all purposes as if fully set forth herein.

In some embodiments, a protein degrader of the present disclosure can be a lysosome-targeting chimera (LYTAC). In some embodiments, a LYTAC described herein may be used, e.g., to target any of various proteins described herein for degradation (e.g., via a lysosomal-based degradation pathway). A LYTAC can target membrane proteins and/or extracellular proteins for degradation, such as is described by Ahn, G. et. Al., Nat Chem Biol, 17(9), 2021, 937-946 (PMID 33767387), Banik, S. M. et. Al., Nature, 584(7820), 2020, 291-297 (PMID 32728216) and Caianiello et al., Nat Chem Biol. 2021 September; 17(9):947-953 (PMID 34413525), each of which is incorporated herein by reference in its entirety and for all purposes as if fully set forth herein. Without wishing to be bound by theory, LYTACs can bridge the extracellular domain of a target protein to a cell-surface lysosomal targeting receptor. The lysosomal targeting receptor can allow for degradation of the protein in a cell type-specific manner.

Molecular glues are small molecule protein degraders that are capable of inducing interactions between an E3 ubiquitin ligase and a protein, thereby resulting in ubiquitination and degradation of the protein. Without wishing to be bound by theory, upon binding to a protein, a molecular glue can induce a conformational change and render the small molecule-protein complex a “neo-substrate” for E3 ligase. Following the formation of a ternary complex, the “neo-substrate” is ubiquitinated, which leads to ubiquitin-proteasome system (UPS)-mediated protein degradation. Non-limiting examples of molecular glues are anti-cancer aryl-sulfonamides, e.g., Indisulam and CR-83 (a CDK inhibitor). In some embodiments, a molecular glue can drive protein degradation by making novel interactions between ubiquitin ligases and neo-substrates.

### Binding Partners for Extracellular Protein Targets

The present disclosure provides antibodies and antigen binding fragments thereof (collectively “binding partners” and each individually a “binding partner”) as described in Koide, S. et al., (2021), Compositions and Methods Comprising Antibodies that Bind to Covalent Peptide Conjugates, WO2022183112A2, the content of which is incorporated herein by reference in its entirety for all purposes. The term “antibody” includes each binding partner format herein. The binding partners bind with specificity to a protein or fragment thereof.

In embodiments, any binding partner of this disclosure comprises at least one chain that comprises a complementary determining region (CDR) that is CDR1, CDR2, or CDR3 from any heavy or light chain amino acid sequence described herein. In certain examples in the present specification, the CDRs are shown in bold font. The amino acid sequences of the CDR sequences are separately encompassed by this disclosure by way of their positions in the described heavy and light chain amino acid sequences. The disclosure includes binding partners that comprise a described heavy chain CDR1, CDR2, and CDR3. The disclosure also includes binding partners that comprise a described light chain CDR1, CDR2, and CDR3.

The disclosure also includes binding partners that comprise a described heavy chain CDR1, CDR2, and CDR3 and a described light chain CDR1, CDR2, and CDR3. For amino acid sequences of this disclosure that include amino acids that comprise purification or protein production tags, such as HIS tags and/or AVI-tags, the disclosure includes the proviso that the sequences of the described tags may be excluded from the amino acid sequences. Amino acids between the described tags may also be excluded.

Binding partners of this disclosure can be provided as intact immunoglobulins or as fragments of immunoglobulins, including but not necessarily limited to antigen-binding (Fab) fragments, Fab′ fragments, (Fab′)2 fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), isolated complementary determining regions (CDRs), single-chain variable fragment (scFv), and other antibody fragments that retain antigen binding function. In embodiments, one or more binding partners are provided as a component of a Bi-specific T-cell engager (BiTE), bispecific killer cell engager (BiKE), CrossMab (e.g., a binding partner containing four different chains; immunoglobulin crossover (also known as Fab domain exchange or CrossMab format) technology (see eg., WO2009/080253; Schaefer et al., Proc. Natl. Acad. Sci. USA, 108:11187-11192 (2011).), or a chimeric antigen receptor (CAR), such as for producing chimeric antigen receptor T cells (e.g., CAR T cells) and CAR natural killer (NK) cells, and killer macrophages. The disclosure includes binding partners that include the described heavy and light chain variable regions.

In embodiments, the binding partners are multivalent. In embodiments, a tri-specific binding partner is provided. In embodiments, cells express at least a segment of one or more binding partners in the form of a CAR. In an embodiment, a binding partner of this disclosure may be provided as a complex with a polynucleotide, such as an RNA polynucleotide, to form an aptamer. In embodiments, a multi-valent binding partner includes one binding component, such as a paratope, that confers specificity to a particular target on a desired cell type, such as any cancer cell marker. In embodiments, a tri-specific leukocyte engager is provided. In embodiments, the binding partners may be part of a molecule that is activated only in the presence of a protease or other enzyme present in a tumor microenvironment, such embodiments being pertinent to, for instance, a probody, examples of which are known in the art, for example in doi: 10.1126/scitranslmed.3006682, doi: 10.1038/s41467-020-16838-w, and doi: 10.1038/s41587-019-0135-x, from which the descriptions of probodies, and protease activation, are incorporated herein by reference. In an embodiment, the disclosure provides a universal hapten that can be grafted onto inhibitors.

In embodiments, a CAR of this disclosure comprises scFv that comprises heavy and light chains as described herein. As is known in the art for previously described CARs, the scFv is present in a contiguous polypeptide that further comprises a CD3zeta chain and a costimulatory domain. In embodiments, the costimulatory domain comprises a 4-IBB costimulatory domain or a CD28 costimulatory domain. A CAR may also contain a coreceptor hinge sequence, such as a CD8 a co-receptor hinge sequence.

In embodiments, binding partners of this disclosure may comprise a constant region, e.g., an Fc region. Any isotype of constant region can be included. Binding partners that comprise a constant region may be particularly adapted for antibody-dependent cell mediated cytotoxicity (ADCC) and thus may function to kill targeted cells by cell-mediated responses by any of a variety of effector cells. Similarly, a constant region may be particularly adapted for enhancing complement-mediated responses.

In embodiments, a binding partner of this disclosure may be modified such that it is present in a fusion protein. In embodiments, an antigen binding segment of a binding partner may be present in a fusion protein, and/or the constant region may be a component of a fusion protein. In embodiments, a fusion protein comprises amino acids from at least two different proteins. Fusion proteins can be produced using any of a wide variety of standard molecular biology approaches, including but not necessarily limited to expression from any suitable expression vector. In embodiments, a binding partner described herein may be present in a fusion protein with a detectable protein, such as green fluorescent protein (GFP), enhanced GFP (eGFP), mCherry, and the like. In embodiments, as an alternative to an expression vector, an mRNA or chemically modified mRNA encoding any binding partner described herein can be delivered to cells such that the binding partner is translated by the cells.

In embodiments, binding partners described herein are used to carry drugs or toxins, and thus the binding partners may be provided as immunotoxins, or in the form of antibody-drug conjugates (ADCs).

In embodiments, agents useful in the generation of immunotoxins include enzymatically active toxins and enzymatically active fragments thereof. Suitable enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. These can be provided as components of fusion proteins or can be covalently attached to the binding partner by any suitable conjugation approach.

The binding partner may be connected to a chemotherapeutic agent by using any suitable linker to form an antibody drug conjugate (ADC). In embodiments, the linker comprises a disulfide, a hydrazine, or a thioether. The chemotherapeutic agent may be reversibly or irreversibly attached to the binding partner.

Cleavable linkers may be particularly useful for killing bystander cells. In embodiments, a protease recognition site may be included to liberate the chemotherapeutic agent from the binding partner by operation of a protease that recognizes and cleaves at the protease recognition site. The ADC may therefore be considered to contain a prodrug.

In embodiments, binding partners of this disclosure may comprise linking sequences. As a non-limiting example, an ScFv may comprise a linker that links segments comprising paratopes to one another. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, and 20 amino acids. In embodiments, a binding partner may include a cellular localization signal, or a secretion signal. In embodiments, binding partner may comprise a transmembrane domain, and thus may be trafficked to, and anchored in a cell membrane. For secretion, any suitable secretion signal can be used, and many are known in the art.

In embodiments, the binding partners can be part of an ADC and therefore the binding partners comprise a drug. The drug can include, but is not necessarily limited to, any suitable chemotherapeutic agent. In embodiments, the ADC comprises a binding partner and a chemotherapeutic agent that is an anti-microtubule agent, an alkylating agent, or a DNA minor groove binding agent. In embodiments, the chemotherapeutic agent comprises a maytansinoid, a dolastatin, an auristatin drug analog, or a cryptophycin. In embodiments, the chemotherapeutic agent is a duocarmycin derivative, or an antibiotic, such as an enediyne antibiotic, or pyrolobenodiazepine (PBD), including dimers thereof. In embodiments, the chemotherapeutic agent is an enzyme inhibitor, such as a topoisomerase or polymerase inhibitor. In embodiments, the chemotherapeutic agent comprises doxorubicin, or a metal-containing compound, such as a platinum-containing compound, non-limiting examples of which include cisplatin, carboplatin or oxaliplatin. In embodiments, the ADC comprises a binding partner described herein, and any drug that is described in Barf and Kaptein, dx.doi.org/10.1021/jm3003203, J. Med. Chem. 2012, 55, 6243-6262, or in Wilson et al., dx.doi.org/10.1021/jm400224q, J. Med. Chem. 2013, 56, 7463-7476, or Lambert and Morris, Adv Ther (2017) 34:1015-1035, from which the descriptions of drugs for use as components as ADCs is incorporated herein by reference. In embodiments, the binding partner is conjugated to or otherwise includes a cytokine, including but not necessarily limited to an interleukin, including but not limited to IL-2 and IL-12, or an interferon (IFN), to thereby provide a cytokine conjugate.

For production of binding partners, any suitable expression system may be used. In general, polynucleotides encoding binding partners are used to express the binding partners in any suitable cell system, non-limiting embodiments of which include NSO murine myeloma cells, human cell lines, and Chinese hamster ovary (CHO) cells. In embodiments, the disclosure provides a polynucleotide that can selectively hybridize to a polynucleotide encoding any CDR or combination of CDRs described herein. In embodiments, the polynucleotide selectively hybridizes to a polynucleotide encoding a heavy chain CDR1, CDR2, and CDR3 of any described binding partner. In embodiments, the polynucleotide selectively hybridizes to a polynucleotide encoding a light chain CDR1, CDR2, and CDR3 of any described binding partner. In embodiments, the polynucleotide selectively hybridizes to a polynucleotide encoding CDR1, CDR2, and CDR3 of a heavy and light chain of any described binding partner.

In embodiments, a binding partner described herein may be a component of a fusion protein. In embodiments, such as for a binding partner that is produced as a fusion protein, a peptide linker may be used. In embodiments, the peptide linker comprises any self-cleaving signal. In embodiments, the self-cleaving signal may be present in the same open reading frame (ORF) as the ORF that encodes the binding partner. A self-cleaving amino acid sequence is typically about 18-22 amino acids long.

To the extent any segment of a protein comprising a binding partner described herein was a component of a library, including but not necessarily limited to a phage display library or a yeast surface display library, the disclosure includes the proviso that the binding partner may be free of any segment of the library that comprises a bacteriophage or yeast amino acid sequence, including but not limited to phage coat protein or a yeast host protein, including but not limited to Aga2. Thus, in certain embodiments, the binding partner may be present in a fusion protein, but the fusion protein does not comprise bacteriophage coat protein. In embodiments, any binding partner described herein may be free of any of pill phage coat protein, or any part of Ml, fd filamentous phage, T4, T7, or 1 phage protein.

In embodiments, a binding partner of this disclosure comprises a detectable label, which may be used for diagnostic or therapeutic purposes. For example, a detectable label can be used for localization of the binding partner for pathology and/or in vivo imaging approaches. In embodiments, a binding partner is conjugated to any of a variety of radioactive agents, including but not limited to a highly radioactive atom, such as Ini 11, At211, 1131, 1125, Y90, Rel86, Rel88, Sml53, Bi212, P32, Pb212, and radioactive isotopes of Lu. In particular embodiments, such as for imaging, the binding partner may be conjugated to a radioactive atom for scintigraphic approaches, for example Tc99m (metastable technetium-99), 1123, or a spin label for nuclear magnetic resonance (NMR) imaging (also known as magnetic resonance imaging, or “MRI”), such as 1123, 1131, 1124, F19, C13, N15, 017 or Gadlinium (III) or Manganese (II). In embodiments, the radioactive agent is suitable for use in CAT scan or PET imaging. In embodiments, Indium 111, Technetium99 or Iodinel31 can be used for planar scans or single photon emission computed tomography (SPECT). Positron emitting labels such as Fluorine19 Iodine 123 and Iodine 124 can be used in positron emission tomography. Paramagnetic ions such as Gadlinium (III) or Manganese (II) can used in magnetic resonance imaging MRI. In embodiments, the described radioactive isotopes that are attached to a described binding partner can also be used in therapeutic approaches. In embodiments, radioactive agents or isotopes include alpha-emitting radionuclides. In embodiments, radioactive agents or isotopes include beta-emitting radionuclides. In some embodiments, the present disclosure provides an antibody of the present technology conjugated to a diagnostic or therapeutic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

Any binding partner described herein may be fully or partially humanized.

Techniques for humanization of antibodies are known in the art and can be adapted for use in the present disclosure. In embodiments, humanization may be performed, for example, by CDR-grafting. In embodiments, for humanization or to otherwise improve a characteristic of the binding partners, one or more amino acids in a variable region can be changed. In embodiments, one or more amino acids in a framework region can be changed.

The disclosure includes binding partners for use in diagnostic and therapeutic approaches. For therapeutic approaches, in certain embodiments, binding partners may be delivered as mRNA or DNA polynucleotides that encode the binding partners. It is considered that administering a DNA or RNA encoding any binding partner described herein is also a method of delivering such binding partners to an individual or one or more cells. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus. In embodiments, a retroviral vector adapted from a murine Moloney leukemia virus (MLV) or a lentiviral vector may be used, such as a lentiviral vector adapted from human immunodeficiency virus type 1 (HIV-1).

In an embodiment, an oncolytic viral vector is used. Oncolytic viruses (OVs), including vaccinia (OVV), mediate anticancer effects by both direct oncolysis and stimulation of innate immune responses through production of damage-associated molecular patterns (DAMPs) and the presence of virus-derived pathogen-associated molecular patterns (PAMPs), leading to increased type I interferon production. Additionally, OW-mediated oncolysis may facilitate the direct acquisition of tumor-derived antigens by host antigen-presenting cells within the tumor microenvironment, thereby leading to improved T cell priming as well as coordination of the effector phase of antitumor immune responses. In alternative embodiments, a recombinant adeno-associated virus (AAV) vector may be used. In certain embodiments, the expression vector is a selfcomplementary adeno-associated virus (scAAV).

Pharmaceutical formulations containing binding partners are included in the disclosure and can be prepared by mixing them with one or more pharmaceutically acceptable carriers. Pharmaceutically acceptable carriers include solvents, dispersion media, isotonic agents, and the like. The carrier can be liquid, semi-solid, e g. pastes, or solid carriers. Examples of carriers include water, saline solutions or other buffers (such as phosphate, citrate buffers), oil, alcohol, proteins (such as serum albumin, gelatin), carbohydrates (such as monosaccharides, di saccharides, and other carbohydrates including glucose, sucrose, trehalose, mannose, mannitol, sorbitol or dextrins), gel, lipids, liposomes, resins, porous matrices, binders, fillers, coatings, stabilizers, preservatives, liposomes, antioxidants, chelating agents such as EDTA, salt forming counter-ions such as sodium; nonionic surfactants such as TWEEN, PLURONICS or polyethylene glycol (PEG), or combinations thereof. In embodiments, a liposomal formulation comprising one or more binding partners is provided. Liposomal formulations include but are not limited to liposomal nanoparticles.

In embodiments, an effective amount of one or more binding partners is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight, and gender of the patient, desired duration of treatment, method of administration, time and frequency of administration, drug combination(s), reaction sensitivities, and/or tolerance/response to therapy.

Binding partners and pharmaceutical compositions comprising the binding partners can be administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. The compositions may be administered parenterally or enterically. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated.

In embodiments, the individual in need of a composition of this disclosure has been diagnosed with or is suspected of having cancer. In embodiments, the cancer is a solid tumor or a hematologic malignancy. In embodiments, the cancer is renal cell carcinoma, breast cancer, prostate cancer, pancreatic cancer, lung cancer, liver cancer, ovarian cancer, cervical cancer, colon cancer, esophageal cancer, glioma, glioblastoma or another brain cancer, stomach cancer, bladder cancer, testicular cancer, head and neck cancer, melanoma or another skin cancer, any sarcoma, including but not limited to fibrosarcoma, angiosarcoma, osteosarcoma, and rhabdomyosarcoma, and any blood cancer, including all types of leukemia, lymphoma, and myeloma. In embodiments, the individual is in need of treatment for any pre-neoplastic disorder, including myelodysplastic syndromes or myeloproliferative neoplasms. In embodiments, a described binding partner is used prophylactically for any of the described types of cancer.

In embodiments, administering one or more binding partners, including but not necessarily in a pharmaceutical formulation, to an individual in need thereof, exhibits an improved activity relative to a control. In an embodiment, the control comprises different antibodies, a different form of the same antibodies/binding partner, or antibodies/binding partners that are delivered without adding additional agents. In embodiments, a binding partner described herein provides for improved antibody dependent cell cytotoxicity (ADCC), or for internalization (such as for an ADC), relative to a control. In embodiments, a control protein or peptide does not comprise the covalently linked molecule. The control peptide may comprise the same sequence as the experimental peptide, or if the experimental peptide comprises a mutation the control peptide may comprise the wild type sequence.

A composition of this disclosure, such as a pharmaceutical formulation, can contain only one, or more than one binding partner, and thus combinations of different binding partners are included. Likewise, one or more binding partners can be combined with any other therapeutic agent, non-limiting examples of which include conventional chemotherapeutic agents, and modulators of T-cell costimulatory molecules, often referred to as immune checkpoint inhibitors. T-cell costimulatory molecules are known in the art (PMID 30115704), including, but not limited to, CTLA4, PD-1, PD-L1, LAG3, TIM3, TIGIT, VISTA, B7-1, B7-2, PD-L2, LSECtin, Galectin-9, CEACAM-1, CD155, CD112, CD28, ICOS, ICOSL, OX40, OX40L, GITR, GITRL, 4-1BB, 4-1BBL, CD40, CD40L, CD27, and CD70. Thus, the disclosure includes combination therapy using one or more described binding partners and any of modulators of T-cell costimulatory molecules, including but not limited to CTLA-4 inhibitors, PD-1 inhibitors and PD-L1 inhibitors. As non-limiting examples, anti-PD-1 agents include Pembrolizumab and Nivolumab. Anti-PD-L1 examples include Avelumab and Atezolizumab. An anti-CTLA-4 example is Ipilimumab. The binding partners may also be combined with any form of adoptive immunotherapy.

In embodiments, the disclosure comprises administering to an individual in need thereof one or more binding partners and at least one additional agent to provide an additive effect, or a greater than additive effect such as a synergistic result. In embodiments, the described effect comprises inhibition of cancer growth, inhibition of metastasis, or other beneficial effect. An additive effect or synergistic effect may also be achieved by using a combination of at least two described binding partners.

Various techniques have been developed for the production of binding partners and are included in the scope of this disclosure. In embodiments, the binding partners are produced by host cells by way of recombinant expression vectors. The present disclosure includes all polynucleotide sequences encoding the amino acid sequences described herein, expression vectors comprising such polynucleotide sequences, and in vitro cell cultures comprising such expression vectors. In embodiments, the cell cultures include prokaryotic cells or eukaryotic cells. In embodiments, the cell cultures are mammalian cells. In embodiments, the cells are CHO cells. In embodiments, the cells are HEK293 cells and their derivatives. Kits comprising the binding partners, and/or cell cultures expressing the binding partners, are provided by this disclosure. In general, the kits comprise one or more sealed containers that contain the binding partners, or cells expressing them. Instructions for using the binding partners for therapeutic and/or diagnostic purposes can be included in the kits.

Cells that are modified to express any described binding partner include but are not necessarily limited CD4+ T cells, CD8+ T cells, Natural Killer T cells, gd T cells, and cells that are progenitors of T cells, such as hematopoietic stem cells or other lymphoid progenitor cells, such as immature thymocytes (double-negative CD4−CD8−) cells, or double-positive thymocytes (CD4+CD8+). In embodiments, the progenitor cells comprise markers, such as CD34, CD117 (c-kit) and CD90 (Thy-1). In embodiments, the modified cells comprise macrophages. The described modified cells may be used therapeutically or prophylactically.

In embodiments, the disclosure provides for generation of a binding partner. This approach comprises providing a plurality of distinct binding partners, exposing the plurality of distinct (e g., different) binding partners to one or a diversity of peptide conjugates, and selecting binding partners that bind with specificity to the peptide conjugates that contain the covalently conjugated drug or other molecule, but do not bind to the protein or peptide that does not comprise the covalently conjugated drug or other molecule. As described above, this approach can be performed on a manner that either does, or does not, require the amino acid sequence of the protein or peptide to be part of the antigenic determinant.

In embodiments, binding partners described herein and as otherwise will be apparent by those skilled in the art, can be used to determine whether or not a particular drug or other molecule forms a covalent interaction with a protein or peptide. Thus, the disclosure provides for exposing protein or peptide substrates to drug candidates and using the binding partners described herein or as identified as described herein to determine whether or not the drug forms a covalent interaction with the pertinent substrate. This determination can be made based on whether or not the binding partner binds to the protein or peptide that has been covalently attached to the drug. This approach can be used in lieu of currently available techniques, such as mass spectroscopy and the like.

In embodiments, binding partners of this disclosure may be used in any immunological diagnostic test, including but not limited to the imaging approaches described above. In embodiments, one or more binding partners described herein can be used as a component in any form of, for example, enzyme-linked immunosorbent assay (ELISA) assay, including but not limited to a direct ELISA, a sandwich ELISA, a competitive ELISA, and a reverse ELISA. In embodiments, one or more binding partners described herein can also be incorporated into an immunodiagnostic device, such as a microfluidic device, a lateral flow device, and the like. The binding partners may also be used in, for example, Western blots and immunoprecipitation assays.

### Pharmaceutical Compositions

### Pharmaceutical Compositions Comprising KRAS Inhibitors

Any of various compositions, e.g., any of various KRAS inhibitors, inhibitors of expression or function or degraders of one or more of various proteins (e.g., VRK1, RIOK2, PKN2, EXT1, EXT2, ELP1, ELP2, ELP3, ELP4, ELP5, ELP6, PGD, PGM2, ROCK1, and ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 proteins) described herein, can be present in a pharmaceutical composition (such as a formulation) that can includes other agents, excipients, or stabilizers. In various embodiments, a pharmaceutical composition described herein may comprise (i) a KRAS inhibitor, (ii) an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, and (iii) a pharmaceutically acceptable carrier and/or excipient. In various embodiments, a pharmaceutical composition described herein may comprise (i) a KRAS inhibitor, (ii) an inhibitor of expression or function or a degrader of one or more proteins selected from VRK1, RIOK2, PKN2, EXT1, EXT2, ELP2, ELP3, ELP5, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2, and (iii) a pharmaceutically acceptable carrier and/or excipient. The KRAS inhibitor can be any of various KRAS inhibitors described herein. As a non-limiting example, in some embodiments, the KRAS inhibitor is a KRAS G12C inhibitor (G12Ci) described herein.

It is understood that any the compounds of the present disclosure can be present in one or more stereoisomers (e.g., diastereomers). The disclosure includes, within its scope, all of these stereoisomers, either isolated (e.g., in enantiomeric isolation) or in combination (including racemic and diastereomeric mixtures). The present disclosure uses amino acids independently selected from L and D forms (e.g., the peptide may contain two serine residues, each serine residue having the same or opposite absolute stereochemistry), etc., are intended for the use of both L- and D-form amino acids.

Accordingly, the compounds of the present disclosure also include substantially pure stereoisomeric form of the specific compound with respect to the asymmetric center of the amino acid residue, for example about 90% de, such as greater than about 95% to 97% de, or 99% de. For larger compounds, as well as mixtures thereof (such as racemic mixtures). Such diastereomers may be prepared, for example, by asymmetric synthesis using chiral intermediates, or the mixture may be divided by conventional methods, such as chromatography or the use of dividing agents.

If the compounds of the disclosure require purification, chromatographic techniques such as high-performance liquid chromatography (HPLC) and reverse phase HPLC can be used. Peptides may be characterized by mass spectrometry and/or other suitable methods.

If the compound contains one or more functional groups that can be protonated or deprotonated (e.g., at physiological pH), the compound can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

In some embodiments, it is envisioned that two or more combinations of the compounds of the disclosure will be administered to the subject. It is believed that the compound (s) may also be administered in combination with one or more additional therapeutic agents. This combination can allow separate, continuous or simultaneous administration with the other active ingredients of the above compounds. This combination may be provided in the form of a pharmaceutical composition.

As used herein, the term “combination” is used by the combination agents as defined above dependently or independently, or by the use of different fixed combinations with different amounts of combination agents, i.e. simultaneously or at different times. Refers to a kit of compositions or parts that can be administered. The combination agents can then be administered, for example, simultaneously or staggered in time (i.e., at different times and at equal or different time intervals for any part of the kit). The ratio of the total amount of combination agents administered in a combination can vary, e.g., to address the needs of a subpopulation of patients to be treated or the needs of a single patient, and different needs are the age of the patient, it can be due to gender, weight, etc.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

In some embodiments, the composition further comprises a targeting agent or a carrier that promotes the delivery of the inhibitors of endocytosis to an area affected by the chronic pain. Exemplary carriers include liposomes, micelles, nanodisperse albumin and its modifications, polymer nanoparticles, dendrimers, inorganic nanoparticles of different compositions.

The appropriate formulation for the compound of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The formulation may also include suitable excipients, such as antioxidants. Examples of antioxidants include phenolic compounds such as BHT or Vitamin E, reducing agents such as methionine or sulfites, and metal chelating agents such as EDTA.

The compounds or pharmaceutically acceptable salts thereof described herein can be prepared in parenteral dosage forms such as those suitable for, e.g., intravascular (intravenous or intraarterial), intraperitoneal, subcutaneous, intradermal, intratumoral, intraventricular, intrapleural or intramuscular administration delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol and). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. Suitable pharmaceutical forms for injection can be delivered by any suitable route, including intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. A dosage unit form may contain a predetermined amount of active substance calculated to produce a desired therapeutic effect described herein. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 g to about 2000 mg. Expressed in proportion, the active compound may be present in a carrier of about 0.25 g to about 2000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the inhibitor of endocytosis. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the inhibitor of endocytosis with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Pharmaceutical Compositions Comprising SHP2 Inhibitors

Any of various compositions, e.g., any of various SHP2 inhibitors, inhibitors of expression or function or degraders of one or more of various proteins (e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 proteins) described herein, can be present in a pharmaceutical composition (such as a formulation) that can includes other agents, excipients, or stabilizers. In various embodiments, a pharmaceutical composition described herein may comprise (i) an SHP2 inhibitor, (ii) an inhibitor of expression or function or a degrader of one or more proteins selected from VRK1, RIOK2, ELP4, and ELP5, and (iii) a pharmaceutically acceptable carrier and/or excipient. In various embodiments, a pharmaceutical composition described herein may comprise (i) an SHP2 inhibitor, (ii) a binding partner of one or more proteins selected from TFRC, SLC7A6OS, GNB2L1, GFER, ATP6AP2, SLC25A19, and PEAR1, and (iii) a pharmaceutically acceptable carrier and/or excipient. The SHP2 inhibitor can be any of various SHP2 inhibitors described herein. As a non-limiting example, in some embodiments, the SHP2 inhibitor is selected from BBP-398, TNO155, RMC-4630, JAB-3068, RLY-1971, and ERAS-601.

It is understood that any the compounds of the present disclosure can be present in one or more stereoisomers (e.g., diastereomers). The disclosure includes, within its scope, all of these stereoisomers, either isolated (e.g., in enantiomeric isolation) or in combination (including racemic and diastereomeric mixtures). The present disclosure uses amino acids independently selected from L and D forms (e.g., the peptide may contain two serine residues, each serine residue having the same or opposite absolute stereochemistry), etc., are intended for the use of both L- and D-form amino acids.

Accordingly, the compounds of the present disclosure also include substantially pure stereoisomeric form of the specific compound with respect to the asymmetric center of the amino acid residue, for example about 90% de, such as greater than about 95% to 97% de, or 99% de. For larger compounds, as well as mixtures thereof (such as racemic mixtures). Such diastereomers may be prepared, for example, by asymmetric synthesis using chiral intermediates, or the mixture may be divided by conventional methods, such as chromatography or the use of dividing agents.

If the compounds of the disclosure require purification, chromatographic techniques such as high-performance liquid chromatography (HPLC) and reverse phase HPLC can be used. Peptides may be characterized by mass spectrometry and/or other suitable methods.

If the compound contains one or more functional groups that can be protonated or deprotonated (e.g., at physiological pH), the compound can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

In some embodiments, it is envisioned that two or more combinations of the compounds of the disclosure will be administered to the subject. It is believed that the compound (s) may also be administered in combination with one or more additional therapeutic agents. This combination can allow separate, continuous or simultaneous administration with the other active ingredients of the above compounds. This combination may be provided in the form of a pharmaceutical composition.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

In some embodiments, the composition further comprises a targeting agent or a carrier that promotes the delivery of the inhibitors of endocytosis to an area affected by the chronic pain. Exemplary carriers include liposomes, micelles, nanodisperse albumin and its modifications, polymer nanoparticles, dendrimers, inorganic nanoparticles of different compositions.

The appropriate formulation for the compound of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The formulation may also include suitable excipients, such as antioxidants. Examples of antioxidants include phenolic compounds such as BHT or Vitamin E, reducing agents such as methionine or sulfites, and metal chelating agents such as EDTA.

The compounds or pharmaceutically acceptable salts thereof described herein can be prepared in parenteral dosage forms such as those suitable for, e.g., intravascular (intravenous or intraarterial), intraperitoneal, subcutaneous, intradermal, intratumoral, intraventricular, intrapleural or intramuscular administration delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol and). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. Suitable pharmaceutical forms for injection can be delivered by any suitable route, including intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. A dosage unit form may contain a predetermined amount of active substance calculated to produce a desired therapeutic effect described herein. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 g to about 2000 mg. Expressed in proportion, the active compound may be present in a carrier of about 0.25 g to about 2000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the inhibitor of endocytosis. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the inhibitor of endocytosis with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Dosage and Administration Regimens

### Dosage and Administration Regimens Comprising KRAS Inhibitors

Provided herein is a method of treating a KRAS mutant cancer in a subject in need thereof, comprising administering to the subject an effective amount of a KRAS inhibitor and an inhibitor of expression or function or a degrader of one or more proteins (e.g., VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2).

Exemplary KRAS inhibitors include, but are not limited to, adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some aspects, a KRAS inhibitor refers to a therapeutic agent capable of detectably lowering the expression of or the activity of the KRAS signaling pathway, compared to a control without treatment with inhibitor. The inhibited expression of or activity level of the KRAS signaling pathway can be 10%, 15%, 20%, 25%, 30%, 35%, 40%, 45%, 50%, 55%, 60%, 65%, 70%, 75%, 80%, 85%, 90%, 95%, or less than that in a control without treatment with inhibitor. In certain aspects, the inhibition is 1.5-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold, or more in comparison to a control. A KRAS inhibitor can inhibit the KRAS signaling pathway (e.g., by partially or totally binding, partially or totally blocking stimulation of the KRAS signaling pathway; by decreasing, preventing, or delaying activation of the KRAS signaling pathway; or inactivating, desensitizing, or down-regulating gene expression, signal transduction, or enzymatic activity of the KRAS signaling pathway.

In some embodiments, the KRAS inhibitor may be any KRAS inhibitor known in the art. In certain aspects, the KRAS inhibitor is a KRAS G12 inhibitor. In some aspects, the KRAS inhibitor is a KRAS G12C inhibitor. In some aspects, the KRAS inhibitor is a KRAS G12V inhibitor. In further aspects, the KRAS inhibitor is a KRAS G12D inhibitor. In some aspects, the KRAS inhibitor is a KRAS G13 inhibitor. In some aspects, the KRAS inhibitor is a G13D inhibitor. In some aspects, the KRAS inhibitor is a Q61H inhibitor. In some aspects, the KRAS inhibitor is a Q61L inhibitor. In some aspects, the KRAS inhibitor is a Q61R inhibitor. In aspects, the KRAS inhibitor is a KRAS H61 inhibitor. In some aspects, the KRAS inhibitor is a H117 inhibitor.

In some embodiments, the method comprises administering to the subject an effective amount of a KRAS inhibitor and an inhibitor of expression or function or a degrader of one or more proteins selected from TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and LARS2.

In some embodiments, the method comprises administering to the subject an effective amount of a KRAS inhibitor and an inhibitor of expression or function or a degrader of one or more proteins selected from AP2S1, ERBB3, GRB2, CRK, SRC, PAK2, EXT2, NDST1, SHOC2, and IPO11.

In some embodiments, the method comprises administering to the subject an effective amount of a KRAS inhibitor and an inhibitor of expression or function or a degrader of MTOR protein and/or RPTOR protein.

In some embodiments, the method comprises administering to the subject an effective amount of a KRAS inhibitor and an inhibitor of expression or function or a degrader of one or more proteins selected from VRK1, PKN2, ELP1, ELP2, ELP3, ELP4, ELP5, ELP6, RIOK2, EXT1, and EXT2.

In some embodiments, the method comprises administering to the subject an effective amount of a KRAS inhibitor and an inhibitor of expression or function or a degrader of VRK1protein, PKN2 protein, and/or RIOK2 protein.

In some embodiments, the method comprises administering to the subject an inhibitor of kinase activity of VRK1protein, PKN2 protein, and/or RIOK2 protein.

In some embodiments, the method comprises administering to the subject an inhibitor of ATPase activity of RIOK2 protein.

In some embodiments, the KRAS inhibitor(s) and/or the inhibitor(s) of expression or function or degrader(s) of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are an antibody, an oligonucleotide, a peptide, an antibody fragment, a ribonucleic acid, or an siRNA. In some embodiments, the KRAS inhibitor(s) and/or the inhibitor(s) of expression or function or degrader(s) of one or more proteins selected from VRK1, RIOK2, PKN2, EXT1, EXT2, ELP1, ELP2, ELP3, ELP4, ELP5, ELP6, PGD, PGM2, ROCK1,ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are a small molecule inhibitor.

In certain embodiments, an amount of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, or pharmaceutical composition thereof, which is administered to a subject as described herein is a therapeutically effective amount.

The phrase “therapeutically effective amount” as used herein, means an amount sufficient to achieve the desired effect for which it is administered. The subject can be from any suitable species, such as mammalian or eukaryotic subjects (e.g., human subject or non-human mammalian subject). A mammal can be, but is not limited to, a non-human mammal, a hamster, a rodent, a mouse, a human, or a rat. Non-human mammals include, but are not limited to, non-human primates, for example, monkeys and apes. The term “non-human” means excluding humans.

In some embodiments, the subject is human. The human can be a patient.

In some embodiments, the subject is a veterinary animal or an experimental model.

In certain embodiments, the method of treating disease is in a patient that has received no prior treatment. In certain embodiments, the method of treating disease is in a patient that has received a prior treatment with therapeutic agent(s), (e.g., chemotherapeutic agent(s)). In certain embodiments, the patent has developed an acquired resistance to the previous treatment of one or more chemotherapeutic agents or immunotherapies. In further embodiments, the patent has developed bypass resistance to the previous treatment of one or more chemotherapeutic agents or immunotherapies.

A chemotherapeutic or chemotherapeutic agent (i.e., an anti-cancer agent), is used according to its ordinary meaning, referring to a chemical composition, biologic, protein, nucleic acid, drug, inhibitor, antagonist, modulator, and/or compound having the ability to inhibit the growth or proliferation of cells and/or antineoplastic properties. In some aspects, an anti-cancer agent is a therapeutic agent approved by the FDA or equivalent or similar regulatory agency, for treating cancer.

Chemotherapeutic agents with which the subject may have been treated prior to treatment with the treatments described herein include, but are not limited to, adrenocorticoids and corticosteroids, kinase inhibitors, androgens, alkylating agents, estrogens, peptide and peptidomimetic signal transduction inhibitors, aclamycin and aclamycin derivatives, pyrimidine analogs, purine analogs, platinum compounds, antiestrogens, antiandrogens, plant alkaloids, antimetabolites, microtubule inhibitors, amanitins, epothilones, mitomycins, discodermolides, dolastatins, inflammatory and proinflammatory agents, camptothecins, and/or immunotherapies.

In certain embodiments, the patient has been administered a prior treatment for NSCLC, such as platinum, abraxane, pembrolizumab, bevacizumab, pemetrexed, platinum doublet, paclitaxel, atezolizumab, carboplatin, and combinations thereof.

In certain embodiments, the patient has been administered a prior treatment for colorectal cancer, such as leucovorin, ramucirumab, cetuximab, bevacizumab, capecitabine, panitumumab, oxaliplatin, ziv-aflibercept, fluorouracil (5-FU), irinotecan, pemborlizumab, binimetinib, ipilimumab, nivolumab, encorafenib, amucirumab, and combinations thereof.

In certain embodiments, the patient has been administered a prior treatment for pancreatic cancer, such as irinotecan, leucovorin, liposomal irinotecan, gemcitabine, erlotinib, abraxane, fluorouracil (5-FU), oxaliplatin, capecitabine, and combinations thereof.

In certain embodiments, the patient has been administered a prior treatment for uterine cancer, such as cisplatin, doxorubicin, liposomal doxorubicin, docetaxel, trastuzumab, fulvestrant, topotecan, paclitaxel, bevacizumab, carboplatin, temsirolimus tamoxifen, an aromatase inhibitor, and combinations thereof.

In the methods of treatment described herein, additional therapeutic agents can be used in combination with the therapeutic agents described herein that are suitable to the disease being treated (e.g., cancer). In some aspects, the provided methods of treatment further include administering an additional therapeutic agent to the subject. The additional therapeutic agent may be a drug, compound, inhibitor, modulator, or antagonist, having the ability to inhibit the growth or proliferation of cells and/or having antineoplastic properties. In some aspects, the additional therapeutic agent can be a chemotherapeutic agent. In further aspects, the additional therapeutic agent is a therapeutic agent approved by the FDA or an equivalent or similar regulatory agency, for treating cancer. In certain aspects, the additional therapeutic agent is radiation therapy. Suitable additional therapeutic agents include, but are not limited to anesthetics, anticonvulsants, analgesics, allosteric inhibitors, antihelmintics, analeptics, antifungals, antirheumatic agents, anticholinesterases, anticholinergic agents, antibiotics, antineoplastic agents, antimuscarinic agents, growth factors, anabolic steroids, hematological agents, antimycobacterial agents, psychotherapeutic agents, antiviral agents, neural blocking agents, immunological agents, anti-inflammatory agents, protease inhibitors, corticosteroids, anticoagulants, antihistamines, antiprotozoal agents, dopaminergics, muscarinics, vitamins, and hormones. The choice of agent and dosage can be determined by one of skill in the art based on several factors, primarily the given disease being treated.

In some embodiments, the KRAS inhibitor and/or the inhibitor of the expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, or pharmaceutical composition thereof, is administered as a weight-based dose to a subject.

A “weight-based dose” (e.g., a dose measured in mg/kg) as used herein, is a dose of the KRAS inhibitor and/or the inhibitor of the expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 that will vary depending on the subject's weight.

In other embodiments, the KRAS inhibitor and/or the inhibitor of the expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, is administered as a fixed dose.

A “fixed dose” (e.g., a dose measured in mg) means that one dose of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, is used consistently for all subjects regardless of any subject-specific factors, e.g., weight.

In some embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered at a dose of about 0.05 mg/kg to about 1 g/kg, about 0.1 mg/kg to about 1 g/kg, about 0.5 mg/kg to about 50 mg/kg, about 0.5 mg/kg to about 25 mg/kg, about 0.5 mg/kg to about 10 mg/kg, about 1.0 mg/kg to about 10 mg/kg, about 1.0 mg/kg to about 5 mg/kg, about 1.0 mg/kg to about 2 mg/kg, about 0.1 mg/kg to about 5 mg/kg, about 0.1 mg/kg to about 1 mg/kg, about 0.1 mg/kg to about 0.75 mg/kg, about 0.1 mg/kg to about 0.5 mg/kg, about 1.25 mg/kg to about 4 mg/kg, about 1.0 mg/kg, about 1.5 mg/kg, about 2.0 mg/kg, about 2.5 mg/kg, about 3.0 mg/kg, about 4.0 mg/kg, or about 5.0 mg/kg.

In some embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered at a dose of about 10 mg to about 100 mg, about 10 mg to about 98 mg, about 10 mg to about 95 mg, about 10 mg to about 92 mg, about 10 mg to about 90 mg, about 10 mg to about 88 mg, about 10 mg to about 85 mg, about 10 mg to about 82 mg, about 10 mg to about 80 mg, about 10 mg to about 78 mg, about 10 mg to about 75 mg, about 10 mg to about 72 mg, about 10 mg to about 70 mg, about 10 mg to about 68 mg, about 10 mg to about 65 mg, about 10 mg to about 62 mg, about 10 mg to about 60 mg, about 10 mg to about 58 mg, about 10 mg to about 55 mg, about 10 mg to about 52 mg, about 10 mg to about 50 mg, about 10 mg to about 48 mg, about 10 mg to about 45 mg, about 10 mg to about 42 mg, about 10 mg to about 40 mg, about 10 mg to about 38 mg, about 10 mg to about 35 mg, about 10 mg to about 32 mg, about 10 mg to about 30 mg, about 10 mg to about 28 mg, about 10 mg to about 25 mg, about 10 mg to about 22 mg, about 10 mg to about 20 mg, about 10 mg to about 18 mg, about 10 mg to about 15 mg, about 10 mg to about 12 mg, about 15 mg to about 100 mg, about 15 mg to about 98 mg, about 15 mg to about 95 mg, about 15 mg to about 92 mg, about 15 mg to about 90 mg, about 15 mg to about 88 mg, about 15 mg to about 85 mg, about 15 mg to about 82 mg, about 15 mg to about 80 mg, about 15 mg to about 78 mg, about 15 mg to about 75 mg, about 15 mg to about 72 mg, about 15 mg to about 70 mg, about 15 mg to about 68 mg, about 15 mg to about 65 mg, about 15 mg to about 62 mg, about 15 mg to about 60 mg, about 15 mg to about 58 mg, about 15 mg to about 55 mg, about 15 mg to about 52 mg, about 15 mg to about 50 mg, about 15 mg to about 48 mg, about 15 mg to about 45 mg, about 15 mg to about 42 mg, about 15 mg to about 40 mg, about 15 mg to about 38 mg, about 15 mg to about 35 mg, about 15 mg to about 32 mg, about 15 mg to about 30 mg, about 15 mg to about 28 mg, about 15 mg to about 25 mg, about 15 mg to about 22 mg, about 15 mg to about 20 mg, about 15 mg to about 18 mg, about 20 mg to about 100 mg, about 20 mg to about 98 mg, about 20 mg to about 95 mg, about 20 mg to about 92 mg, about 20 mg to about 90 mg, about 20 mg to about 88 mg, about 20 mg to about 85 mg, about 20 mg to about 82 mg, about 20 mg to about 80 mg, about 20 mg to about 78 mg, about 20 mg to about 75 mg, about 20 mg to about 72 mg, about 20 mg to about 70 mg, about 20 mg to about 68 mg, about 20 mg to about 65 mg, about 20 mg to about 62 mg, about 20 mg to about 60 mg, about 20 mg to about 58 mg, about 20 mg to about 55 mg, about 20 mg to about 52 mg, about 20 mg to about 50 mg, about 20 mg to about 48 mg, about 20 mg to about 45 mg, about 20 mg to about 42 mg, about 20 mg to about 40 mg, about 20 mg to about 38 mg, about 20 mg to about 35 mg, about 20 mg to about 32 mg, about 20 mg to about 30 mg, about 20 mg to about 28 mg, about 20 mg to about 25 mg, about 20 mg to about 22 mg, about 25 mg to about 100 mg, about 25 mg to about 98 mg, about 25 mg to about 95 mg, about 25 mg to about 92 mg, about 25 mg to about 90 mg, about 25 mg to about 88 mg, about 25 mg to about 85 mg, about 25 mg to about 82 mg, about 25 mg to about 80 mg, about 25 mg to about 78 mg, about 25 mg to about 75 mg, about 25 mg to about 72 mg, about 25 mg to about 70 mg, about 25 mg to about 68 mg, about 25 mg to about 65 mg, about 25 mg to about 62 mg, about 25 mg to about 60 mg, about 25 mg to about 58 mg, about 25 mg to about 55 mg, about 25 mg to about 52 mg, about 25 mg to about 50 mg, about 25 mg to about 48 mg, about 25 mg to about 45 mg, about 25 mg to about 42 mg, about 25 mg to about 40 mg, about 25 mg to about 38 mg, about 25 mg to about 35 mg, about 25 mg to about 32 mg, about 25 mg to about 30 mg, about 25 mg to about 28 mg, about 30 mg to about 100 mg, about 30 mg to about 98 mg, about 30 mg to about 95 mg, about 30 mg to about 92 mg, about 30 mg to about 90 mg, about 30 mg to about 88 mg, about 30 mg to about 85 mg, about 30 mg to about 82 mg, about 30 mg to about 80 mg, about 30 mg to about 78 mg, about 30 mg to about 75 mg, about 30 mg to about 72 mg, about 30 mg to about 70 mg, about 30 mg to about 68 mg, about 30 mg to about 65 mg, about 30 mg to about 62 mg, about 30 mg to about 60 mg, about 30 mg to about 58 mg, about 30 mg to about 55 mg, about 30 mg to about 52 mg, about 30 mg to about 50 mg, about 30 mg to about 48 mg, about 30 mg to about 45 mg, about 30 mg to about 42 mg, about 30 mg to about 40 mg, about 30 mg to about 38 mg, about 30 mg to about 35 mg, about 30 mg to about 32 mg, about 35 mg to about 100 mg, about 35 mg to about 98 mg, about 35 mg to about 95 mg, about 35 mg to about 92 mg, about 35 mg to about 90 mg, about 35 mg to about 88 mg, about 35 mg to about 85 mg, about 35 mg to about 82 mg, about 35 mg to about 80 mg, about 35 mg to about 78 mg, about 35 mg to about 75 mg, about 35 mg to about 72 mg, about 35 mg to about 70 mg, about 35 mg to about 68 mg, about 35 mg to about 65 mg, about 35 mg to about 62 mg, about 35 mg to about 60 mg, about 35 mg to about 58 mg, about 35 mg to about 55 mg, about 35 mg to about 52 mg, about 35 mg to about 50 mg, about 35 mg to about 48 mg, about 35 mg to about 45 mg, about 35 mg to about 42 mg, about 35 mg to about 40 mg, about 35 mg to about 38 mg, about 40 mg to about 100 mg, about 40 mg to about 98 mg, about 40 mg to about 95 mg, about 40 mg to about 92 mg, about 40 mg to about 90 mg, about 40 mg to about 88 mg, about 40 mg to about 85 mg, about 40 mg to about 82 mg, about 40 mg to about 80 mg, about 40 mg to about 78 mg, about 40 mg to about 75 mg, about 40 mg to about 72 mg, about 40 mg to about 70 mg, about 40 mg to about 68 mg, about 40 mg to about 65 mg, about 40 mg to about 62 mg, about 40 mg to about 60 mg, about 40 mg to about 58 mg, about 40 mg to about 55 mg, about 40 mg to about 52 mg, about 40 mg to about 50 mg, about 40 mg to about 48 mg, about 40 mg to about 45 mg, about 40 mg to about 42 mg, about 45 mg to about 100 mg, about 45 mg to about 98 mg, about 45 mg to about 95 mg, about 45 mg to about 92 mg, about 45 mg to about 90 mg, about 45 mg to about 88 mg, about 45 mg to about 85 mg, about 45 mg to about 82 mg, about 45 mg to about 80 mg, about 45 mg to about 78 mg, about 45 mg to about 75 mg, about 45 mg to about 72 mg, about 45 mg to about 70 mg, about 45 mg to about 68 mg, about 45 mg to about 65 mg, about 45 mg to about 62 mg, about 45 mg to about 60 mg, about 45 mg to about 58 mg, about 45 mg to about 55 mg, about 45 mg to about 52 mg, about 45 mg to about 50 mg, about 45 mg to about 48 mg, about 50 mg to about 100 mg, about 50 mg to about 98 mg, about 50 mg to about 95 mg, about 50 mg to about 92 mg, about 50 mg to about 90 mg, about 50 mg to about 88 mg, about 50 mg to about 85 mg, about 50 mg to about 82 mg, about 50 mg to about 80 mg, about 50 mg to about 78 mg, about 50 mg to about 75 mg, about 50 mg to about 72 mg, about 50 mg to about 70 mg, about 50 mg to about 68 mg, about 50 mg to about 65 mg, about 50 mg to about 62 mg, about 50 mg to about 60 mg, about 50 mg to about 58 mg, about 50 mg to about 55 mg, about 50 mg to about 52 mg, about 55 mg to about 100 mg, about 55 mg to about 98 mg, about 55 mg to about 95 mg, about 55 mg to about 92 mg, about 55 mg to about 90 mg, about 55 mg to about 88 mg, about 55 mg to about 85 mg, about 55 mg to about 82 mg, about 55 mg to about 80 mg, about 55 mg to about 78 mg, about 55 mg to about 75 mg, about 55 mg to about 72 mg, about 55 mg to about 70 mg, about 55 mg to about 68 mg, about 55 mg to about 65 mg, about 55 mg to about 62 mg, about 55 mg to about 60 mg, about 55 mg to about 58 mg, about 60 mg to about 100 mg, about 60 mg to about 98 mg, about 60 mg to about 95 mg, about 60 mg to about 92 mg, about 60 mg to about 90 mg, about 60 mg to about 88 mg, about 60 mg to about 85 mg, about 60 mg to about 82 mg, about 60 mg to about 80 mg, about 60 mg to about 78 mg, about 60 mg to about 75 mg, about 60 mg to about 72 mg, about 60 mg to about 70 mg, about 60 mg to about 68 mg, about 60 mg to about 65 mg, about 60 mg to about 62 mg, about 65 mg to about 100 mg, about 65 mg to about 98 mg, about 65 mg to about 95 mg, about 65 mg to about 92 mg, about 65 mg to about 90 mg, about 65 mg to about 88 mg, about 65 mg to about 85 mg, about 65 mg to about 82 mg, about 65 mg to about 80 mg, about 65 mg to about 82 mg, about 65 mg to about 75 mg, about 65 mg to about 72 mg, about 65 mg to about 70 mg, about 65 mg to about 68 mg, about 70 mg to about 100 mg, about 70 mg to about 98 mg, about 70 mg to about 95 mg, about 70 mg to about 92 mg, about 70 mg to about 90 mg, about 70 mg to about 88 mg, about 70 mg to about 85 mg, about 70 mg to about 82 mg, about 70 mg to about 80 mg, about 70 mg to about 78 mg, about 70 mg to about 75 mg, about 70 mg to about 72 mg, about 75 mg to about 100 mg, about 75 mg to about 98 mg, about 75 mg to about 95 mg, about 75 mg to about 92 mg, about 75 mg to about 90 mg, about 75 mg to about 88 mg, about 75 mg to about 85 mg, about 75 mg to about 82 mg, about 75 mg to about 80 mg, about 75 mg to about 78 mg, about 80 mg to about 100 mg, about 80 mg to about 98 mg, about 80 mg to about 95 mg, about 80 mg to about 92 mg, about 80 mg to about 90 mg, about 80 mg to about 88 mg, about 80 mg to about 85 mg, about 80 mg to about 82 mg, about 85 mg to about 100 mg, about 85 mg to about 98 mg, about 85 mg to about 95 mg, about 85 mg to about 92 mg, about 85 mg to about 90 mg, about 85 mg to about 88 mg, about 90 mg to about 100 mg, about 90 mg to about 98 mg, about 90 mg to about 95 mg, about 90 mg to about 92 mg, about 95 mg to about 100 mg, about 95 mg to about 98 mg, about 10 mg, about 12 mg, about 15 mg, about 18 mg, about 20 mg, about 22 mg, about 25 mg, about 28 mg, about 30 mg, about 32 mg, about 35 mg, about 38 mg, about 40 mg, about 42 mg, about 45 mg, about 48 mg, about 50 mg, about 52 mg, about 55 mg, about 58 mg, about 60 mg, about 62 mg, about 65 mg, about 68 mg, about 70 mg, about 72 mg, about 75 mg, about 78 mg, about 80 mg, about 82 mg, about 85 mg, about 88 mg, about 90 mg, about 92 mg, about 95 mg, about 98 mg, or about 100 mg.

In certain embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered to a subject at a dosing frequency of about five times a week, about four times a week, about twice a week, about once a week, about once every two weeks, about once every three weeks, about once a month, about once every five weeks, about once every six weeks, about once every seven weeks, about once every two months, about once every three months, or less frequently so long as an effective therapeutic response is achieved. In certain embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 can be administered to a subject at a dosing frequency of about six times a year, about four times a year, about twice a year, about once a year, about once every two years, about once every three years, about once every four years, about once every five years, about once every six years, about once every eight years, about once a decade, about once every twelve years, about once every fifteen years, or less frequently so long as an effective therapeutic response is achieved.

Dosage ranges and frequency of administration of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 as described herein can vary depending on the parameters of a specific subject, the route of administration, the type of medical condition, and/or the severity of the medical condition. A more accurate dose may depend on the subject in which it is administered, e.g., a lower dose may be used if the subject in which it is administered is juvenile and a higher dose may be used if the subject is adult. In some embodiments, a more accurate dose may depend on the subject weight.

In certain embodiments, multiple doses of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are administered over a defined time course.

In some embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered following a repeated dosing regimen wherein the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered an initial time (e.g., as an initial dose) and then may be re-administered at any amount for any number of subsequent times thereafter over the time course of treatment. For example, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be re-administered one time, two times, three times, four times, five times, six times, seven times, eight times, nine times, ten times, fifteen times, twenty times, or more, over the time course of treatment which may occur over any amount of time (e.g., days, weeks, or years). In certain embodiments, when the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 the inhibitor(s) which is/are administered first in a repeat dosing regimen may comprise the same inhibitor(s) which is/are re-administered second or thereafter in the regimen, for any number of subsequent times thereafter. In certain embodiments, when the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 the inhibitor(s) which is/are administered first in a repeat dosing regimen may comprise a different inhibitor(s) than is/are re-administered second or thereafter in the regimen, for any number of subsequent times thereafter.

In certain embodiments, the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered using a stepwise dosing regimen. Stepwise dosing can refer to dividing dosing of the same inhibitor(s) over multiple administrations. In certain embodiments, the dosing of the same inhibitor(s) is/are broken up one time, two times, three times, four times, five times, six times, seven times, eight times, nine times, ten times, fifteen times, twenty times, or more, over the time course of the treatment which can occur over any period of time (e.g., over any number of days, weeks, or years). In certain embodiments, when a stepwise dose regimen is used in the administration of the inhibitor(s), the regimen may encompass a increase or decrease in dosage levels with each administration of the inhibitor(s).

In some embodiments, the KRAS inhibitor and the inhibitor of expression or function or degrader of said one or more proteins are administered simultaneously.

In some embodiments, the KRAS inhibitor and the inhibitor of expression or function or degrader of said one or more proteins are administered simultaneously in one composition.

In some embodiments, the KRAS inhibitor and the inhibitor of expression or function or degrader of said one or more proteins are administered simultaneously in different compositions.

In some embodiments, the KRAS inhibitor and the inhibitor of expression or function or degrader of said one or more proteins are administered sequentially.

In certain embodiments, the KRAS inhibitor is administered to a subject in combination with an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. The KRAS inhibitor can be administered prior to, simultaneously with, and/or after the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, when the KRAS inhibitor is administered prior to, simultaneously with, and/or after the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, the KRAS inhibitor is administered one time, two times, three times, four times, five times, six times, seven times, eight times, nine times, ten times, twelve times, fourteen times, fifteen times, eighteen times, or twenty times or more, prior to, simultaneously with, and/or after the administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In certain embodiments, when a KRAS inhibitor is administered with a repeat dosing regimen, an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered once, twice, or any number of times prior to, simultaneously with, and/or after a first, second, and/or any number of subsequent administrations of the KRAS inhibitor thereafter.

In some embodiments, a KRAS inhibitor may be administered to a subject separately from an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, when a KRAS inhibitor and an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are administered separately, the KRAS inhibitor may also be administered simultaneously with the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In certain embodiments, the KRAS inhibitor is administered one or multiple times during the administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In another embodiment, the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered one or multiple times during the administration of the KRAS inhibitor.

In certain embodiments, a KRAS inhibitor and an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered separately over a defined time period. In some embodiments, multiple doses of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered to a subject through a defined time course. The methods may comprise sequentially administering to a subject multiple doses of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In certain embodiments, when the KRAS inhibitor and inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are administered sequentially, the KRAS inhibitor can be administered either before and/or in between each of the administrations of the inhibitor(s) of expression or function or the degrader(s) of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

The term “sequentially administering”, as used herein, means that each dose of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, and ROCK2, is administered to the subject at various points in time, e.g., on different days separated by an interval that may comprise hours, days, weeks, months, or years. In certain embodiments, the methods comprise sequentially administering to the subject a single initial dose of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, followed by one or multiple secondary doses of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, and optionally followed by one or multiple tertiary doses of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

As used herein, the terms “initial dose,” “secondary dose(s),” and “tertiary dose(s)” refer to the sequential sequence of administration of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. Thus, the “initial dose” is the dose administered at the beginning of the treatment regimen schedule (i.e., the “loading dose”), the “secondary dose(s)” is/are the dose(s) which are administered after the “initial dose”, and the “tertiary dose(s)” is/are the dose(s) which are administered after the “secondary dose(s)”.

In certain embodiments, the initial, secondary, and tertiary doses may all contain the same amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 but may differ from one another in the frequency of administration. In another embodiment, the amounts of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 contained in the initial, secondary, and/or tertiary doses varies from one another (e.g., increased or decreased as appropriate) during the course of treatment. In some embodiments, one or multiple (e.g., 1, 2, 3, 4, 5, 6, 7, 8, 9, 10, or more) doses are administered in the beginning of the treatment regimen as loading doses (i.e., initial doses) followed by subsequent doses (e.g., secondary or tertiary doses) that are administered less frequently, known as “maintenance doses”. In certain embodiments, the initial dose and the one or more subsequent doses (i.e., secondary or tertiary doses) each contain the same amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In some embodiments, the initial dose comprises an initial amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 and the one or multiple secondary doses each comprise a different amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In one embodiment, the first amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 can be 1.5×, 2×, 2.5×, 3×, 3.5×, 4×, 4.5×, 5×, 10×, or more than the second amount of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, each secondary dose(s) and/or tertiary dose(s) is/are administered 1 to 20 (e.g., 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5 or more) weeks after the immediately preceding dose. As used herein, the phrase “the immediately preceding dose,” means, in a sequence of multiple administrations, the dose of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2 AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 that is administered to a subject before the administration of the very next subsequent dose in the sequence with no intervening doses.

The methods may comprise administering to a subject any number of secondary doses and/or tertiary doses of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In some embodiments, only one secondary dose is administered to the subject. In certain embodiments, two or more secondary doses are administered to the subject. Likewise, in some embodiments, only one tertiary dose is administered to the subject. In some embodiments, two or more tertiary doses are administered to the subject.

In certain embodiments that involve multiple secondary doses, all secondary doses are administered at the same frequency as one another (e.g., each secondary dose may be administered 1, 2, 3, or 4 weeks, or some other frequency, after the immediately preceding dose. Similarly, in certain embodiments that involve multiple tertiary doses, all tertiary doses are administered at the same frequency as one another. Alternatively, the frequency at which the secondary doses and/or tertiary doses are administered can vary over the treatment regimen course. The frequency of administration may be adjusted during the treatment regimen course by a physician depending on the subject needs following clinical examination.

In certain embodiments, when a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are sequentially administered, the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be a first component of the dosing regimen and the KRAS inhibitor may be a second component of the dosing regimen (i.e., the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered before the KRAS inhibitor). In certain embodiments, the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be a second component of the dosing regimen and the KRAS inhibitor may be a first component of a dosing regimen (i.e., the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 may be administered after the KRAS inhibitor). In other embodiments, an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 and a KRAS inhibitor may be sequentially administered, by using either of the above-described orders with variable time intervals between administrations. For example, the time interval between administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 and the KRAS inhibitor may be at least about 30 seconds, at least about 45 seconds, at least about 1 minute, at least about 2 minutes, at least about 5 minutes, at least about 10 minutes, at least about 15 minutes, at least about 20 minutes, at least about 30 minutes, at least about 45 minutes, at least about 1 hour, at least about 2 hours, at least about 3 to 4 hours, at least about 5 hours, at least about 6 to 7 hours, at least about 8 to 9 hours, at least about 10 hours, at least about 11 to 12 hours, at least about 13 to 14 hours, at least about 15 hours, at least about 16 to 17 hours, at least about 18 to 19 hours, at least about 20 hours, at least about 21 to 24 hours, at least about 1 day, at least about 2 days, at least about 3 to 4 days, at least about 5 days, at least about 6 to 7 days, at least about 8 to 9 days, at least about 10 days, at least about 11 to 12 days, at least about 13 to 14 days, at least about 15 days, at least about 16 to 17 days, at least about 18 to 19 days, at least about 20 days, at least about 21 to 25 days, at least about 26 to 31 days, at least about 1 month, at least about 2 to 5 months, at least about 6 months, at least about 7 to 11 months, at least about 1 year, at least about 2 to 4 years, at least about 5 years, at least about 6 to 9 years, at least about 10 years, or more.

Any of the methods of administration as described herein can further comprise any of various subsequent administration steps as described herein. For example, the subsequent administration step can comprise administering the KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 to the subject one or more subsequent times until a desired clinical outcome and/or measurement of interest is achieved in the subject.

For example, the subsequent administration step(s) can be at least about 1 week, at least about 2 to 4 weeks, at least about 5 weeks, at least about 6 to 9 weeks, at least about 10 weeks, at least about 11 to 15 weeks, or at least about 16 to 20 weeks after the initial dosing.

In some embodiments, the subsequent administration step can comprise administering a second KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 (e.g., that is different from a first KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 administered in an initial administration step) to the subject one or more subsequent times until a desired clinical outcome and/or measurement of interest is achieved in the subject.

For example, the subsequent administration step(s) can be at least about 1 week, at least about 2 to 4 weeks, at least about 5 weeks, at least about 6 to 9 weeks, at least about 10 weeks, at least about 11 to 15 weeks, or at least about 16 to 20 weeks after the initial dosing.

In some embodiments, the subsequently administered KRAS inhibitor and/or inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered via the same administration route as the originally administered KRAS inhibitor and/or inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the subsequently administered KRAS inhibitor and/or inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 is administered via a different administration route as the originally administered KRAS inhibitor and/or inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, the KRAS inhibitor is administered before the administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, the KRAS inhibitor is administered simultaneously with the administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, the KRAS inhibitor is administered after the administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the inhibitors of expression or function or degraders of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 are administered two or more times and the KRAS inhibitor is administered before and/or between each of the administrations of the inhibitors of expression or function or degraders of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the KRAS inhibitor is administered simultaneously with the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 or not simultaneously (e.g., sequentially in any combination). For example, in a method comprising administering a KRAS inhibitor and an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 they can be administered separately (e.g., the KRAS inhibitor separately from the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2). Further, the KRAS inhibitor can be administered prior to, subsequent to, prior to and subsequent to, or at the same time as the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In certain embodiments, the KRAS inhibitor can be administered about 2 hours to about 72 hours, about 2 hours to about 48 hours, about 2 hours to about 24 hours, about 2 hours to about 12 hours, about 2 hours to about 6 hours, about 2 hours to about 4 hours, about 1 hour to about 72 hours, about 1 hour to about 48 hours, about 1 hour to about 24 hours, about 1 hour to about 12 hours, about 1 hour to about 6 hours, about 1 hour to about 4 hours, about 1 hour to about 2 hours, about 6 hours to about 48 hours, about 6 hours to about 24 hours, about 12 hours to about 48 hours, or about 24 hours to about 48 hours prior to and/or subsequent to administration of the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the KRAS inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to administering the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In another embodiment, the KRAS inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and/or subsequent to administering the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the KRAS inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to and subsequent to administering the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In another embodiment, the KRAS inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and subsequent to administering the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

In some embodiments, the KRAS inhibitor is administered at least 2 hours, at least about 4 hours, at least 6 hours, at least about 8 hours, at least about 12 hours, at least 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks after administering inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2. In another embodiment, the KRAS inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days after administering the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2.

Administration in vivo can be by any suitable route including, but not limited to, oral, parenteral, intrathecal, intra-arterial, intraperitoneal, intravenous, subcutaneous, topical, intracranial, intratumoral, intranasal, or intramuscular. Systemic modes of administration include, but are not limited to, parenteral and oral routes. Parenteral routes include, but are not limited to, subcutaneous, intraosseous, intravenous, intranasal, intradermal, intraarterial, intramuscular, and intraperitoneal routes. Some specific examples are intravenous infusion, nasal instillation, and intravitreal injection. Local modes of administration include, but are not limited to, intracerebroventricular, intrathecal, intraparenchymal (e.g., localized intraparenchymal delivery to the cerebral cortex, temporal cortex, striatum, tegmentum, precentral gyrus, hippocampus, thalamus, frontal cortex, hypothalamus, cerebellum, amygdala, medulla, tectum, or substantia nigra), intravitreal, subconjunctival, intraocular, subretinal, intraorbital, and transscleral routes. Significantly smaller amounts of the active components may elicit an effect(s) when administered locally (e.g., intravitreal or intraparenchymal) compared to when administered systemically (e.g., intravenously). Administration locally may also reduce or eliminate the prevalence of potentially toxic side effects that may occur when the component(s) are administered systemically.

Administration in vivo can be by any suitable route including, but not limited to, intravenous, intracranial, subcutaneous, parenteral, intratumoral, intraperitoneal, oral, intra-arterial, intranasal, intrathecal, topical, or intramuscular.

The number of dosages and the frequency of administration can depend several factors. The administration of a KRAS inhibitor and/or an inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 into the cell or subject can be performed one or more times over a specific period of time. In some embodiments, the administration can be performed only once over a period of time, at least two to three times over a period of time, at least four to five times over a period of time, at least six to seven times over a period of time, at least eight to nine times over a period of time, at least ten times over a period of time, at least eleven to twelve times over a period of time, at least thirteen to fourteen times over a period of time, at least fifteen times over a period of time, at least sixteen to nineteen times over a period of time, at least twenty times over a period of time, or at least twenty-two to thirty times over a period of time. In some methods, a single administration of the KRAS inhibitor and/or the inhibitor of expression or function or a degrader of one or more proteins selected from VRK1, RIOK2, PKN2, EXT1, EXT2, ELP1, ELP2, ELP3, ELP4, ELP5, ELP6, PGD, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, is sufficient to achieve the desired effect(s). In some embodiments, more than one administration may be beneficial to maximize desired therapeutic effect.

In some embodiments, the KRAS inhibitor and/or the inhibitor of expression or function or degrader of said one or more proteins is administered orally or intravenously.

In some embodiments, the KRAS inhibitor and/or the inhibitor of expression or function or degrader of said one or more proteins may be carried out in combination with one or more of a known therapy including, but not limited to, surgery, radiation therapy, chemotherapy, photodynamic therapy, and/or immunotherapy.

In some embodiments, the KRAS inhibitor is a KRAS G12C inhibitor (G12Ci).

In some embodiments, the KRAS inhibitor is selected from adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

In some embodiments, the KRAS inhibitor is the KRAS G12C inhibitor (G12Ci) adagrasib (MRTX-849) or sotorasib (AMG510).

Non-limiting examples of an inhibitor of expression or function or degrader of ROCK1 protein and/or ROCK2 protein are AR-12286, Fasudil, Ripasudil, Netarsudil, KD025 (Belumosudil), AT13148, GSK269962, H1152, GSK429286, pharmaceutically acceptable salts thereof, and any combinations thereof.

Non-limiting examples of an inhibitor of expression or function or degrader of MTOR protein and/or RPTOR protein are Rapamycin, RAD001, TORIN, pharmaceutically acceptable salts thereof, and any combinations thereof.

Non-limiting examples of an inhibitor of expression or function or degrader of SRC protein are Dasatanib, Bosutinib, Sarcatanib, pharmaceutically acceptable salts thereof, and any combinations thereof.

In some embodiments, the KRAS mutant cancer is resistant to a treatment with said KRAS inhibitor when said KRAS inhibitor is administered in the absence of said inhibitor of expression or function or degrader of said one or more proteins.

### Dosage and Administration Regimens Comprising SHP2 Inhibitors

Provided herein is a method of treating a KRAS mutant cancer in a subject in need thereof, comprising administering to the subject an effective amount of an SHP2 inhibitor and an inhibitor of expression or function or a degrader of one or more proteins (e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1).

Also provided herein, is a method of treating a KRAS mutant cancer in a subject in need thereof, comprising administering to the subject an effective amount of an SHP2 inhibitor and a binding partner, wherein said binding partner specifically binds to one or more proteins selected from TFRC, SLC7A6OS, GNB2L1, GFER, ATP6AP2, SLC25A19, and PEAR1.

Exemplary SHP2 inhibitors include, but are not limited to, BBP-398, TNO155, RMC-4630, JAB-3068, RLY-1971, and ERAS-601, pharmaceutically acceptable salts thereof, and any combinations thereof.

In some aspects, an SHP2 inhibitor refers to a therapeutic agent capable of detectably lowering the expression of or the activity of the SHP2 signaling pathway, compared to a control without treatment with inhibitor. The inhibited expression of or activity level of the SHP2 signaling pathway can be 10%, 15%, 20%, 25%, 30%, 35%, 40%, 45%, 50%, 55%, 60%, 65%, 70%, 75%, 80%, 85%, 90%, 95%, or less than that in a control without treatment with inhibitor. In certain aspects, the inhibition is 1.5-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold, or more in comparison to a control. An SHP2 inhibitor can inhibit the SHP2 signaling pathway (e.g., by partially or totally binding, partially or totally blocking stimulation of the SHP2 signaling pathway; by decreasing, preventing, or delaying activation of the SHP2 signaling pathway; or inactivating, desensitizing, or down-regulating gene expression, signal transduction, or enzymatic activity of the SHP2 signaling pathway.

In some embodiments, the SHP2 inhibitor may be any SHP2 inhibitor known in the art. In certain aspects, the SHP2 inhibitor is selected from BBP-398, TNO155, RMC-4630, JAB-3068, RLY-1971, and ERAS-601.

In some embodiments, the method comprises administering to the subject an effective amount of an SHP2 inhibitor and an inhibitor of expression or function or a degrader of one or more proteins selected from VRK1, RIOK2, ELP4, and ELP5.

In some embodiments, the method comprises administering to the subject an effective amount of an SHP2 inhibitor and an inhibitor of function of one or more proteins selected from TFRC, SLC7A6OS, GNB2L1, GFER, ATP6AP2, SLC25A19, and PEAR1.

In some embodiments, the SHP2 inhibitor(s) and/or the inhibitor(s) of expression or function or degrader(s) of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are an antibody, an oligonucleotide, a peptide, an antibody fragment, a ribonucleic acid, or an siRNA. In some embodiments, the SHP2 inhibitor(s) and/or the inhibitor(s) of expression or function or degrader(s) of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are a small molecule inhibitor.

In some embodiments, the SHP2 inhibitor(s) and/or the binding partner(s) are an antibody, an oligonucleotide, a peptide, an antibody fragment, a ribonucleic acid, or an siRNA. In some embodiments, the SHP2 inhibitor(s) and/or the binding partner(s) are a small molecule inhibitor.

In certain embodiments, an amount of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, or pharmaceutical composition thereof, which is administered to a subject as described herein is a therapeutically effective amount.

In certain embodiments, an amount of an SHP2 inhibitor and/or a binding partner, which is administered to a subject as described herein is a therapeutically effective amount.

In some embodiments, the subject is human. The human can be a patient.

In some embodiments, the subject is a veterinary animal or an experimental model.

In certain embodiments, the method of treating disease is in a patient that has received no prior treatment. In certain embodiments, the method of treating disease is in a patient that has received a prior treatment with therapeutic agent(s), (e.g., chemotherapeutic agent(s)). In certain embodiments, the patent has developed an acquired resistance to the previous treatment of one or more chemotherapeutic agents or immunotherapies. In further embodiments, the patent has developed bypass resistance to the previous treatment of one or more chemotherapeutic agents or immunotherapies.

A chemotherapeutic or chemotherapeutic agent (i.e., an anti-cancer agent), is used according to its ordinary meaning, referring to a chemical composition, biologic, protein, nucleic acid, drug, inhibitor, antagonist, modulator, and/or compound having the ability to inhibit the growth or proliferation of cells and/or antineoplastic properties. In some aspects, an anti-cancer agent is a therapeutic agent approved by the FDA or equivalent or similar regulatory agency, for treating cancer.

Chemotherapeutic agents with which the subject may have been treated prior to treatment with the treatments described herein include, but are not limited to, adrenocorticoids and corticosteroids, kinase inhibitors, androgens, alkylating agents, estrogens, peptide and peptidomimetic signal transduction inhibitors, aclamycin and aclamycin derivatives, pyrimidine analogs, purine analogs, platinum compounds, antiestrogens, antiandrogens, plant alkaloids, antimetabolites, microtubule inhibitors, amanitins, epothilones, mitomycins, discodermolides, dolastatins, inflammatory and proinflammatory agents, camptothecins, and/or immunotherapies.

In certain embodiments, the patient has been administered a prior treatment for NSCLC, such as platinum, abraxane, pembrolizumab, bevacizumab, pemetrexed, platinum doublet, paclitaxel, atezolizumab, carboplatin, and combinations thereof.

In certain embodiments, the patient has been administered a prior treatment for colorectal cancer, such as leucovorin, ramucirumab, cetuximab, bevacizumab, capecitabine, panitumumab, oxaliplatin, ziv-aflibercept, fluorouracil (5-FU), irinotecan, pemborlizumab, binimetinib, ipilimumab, nivolumab, encorafenib, amucirumab, and combinations thereof.

In certain embodiments, the patient has been administered a prior treatment for pancreatic cancer, such as irinotecan, leucovorin, liposomal irinotecan, gemcitabine, erlotinib, abraxane, fluorouracil (5-FU), oxaliplatin, capecitabine, and combinations thereof.

In some embodiments, an inhibitor of thymidylate synthase (TYMS) can be used in combination with fluorouracil (5-FU). In certain embodiments, the inhibitor of thymidylate synthase (TYMS) and the fluorouracil (5-FU) are administered simultaneously. In certain embodiments, the inhibitor of thymidylate synthase (TYMS) and the fluorouracil (5-FU) are administered simultaneously in one composition. In certain embodiments, the inhibitor of thymidylate synthase (TYMS) and the fluorouracil (5-FU) are administered simultaneously in different compositions. In certain embodiments, the inhibitor of thymidylate synthase (TYMS) and the fluorouracil (5-FU) are administered sequentially. In certain embodiments, the inhibitor of thymidylate synthase (TYMS) and the fluorouracil (5-FU) are administered orally or intravenously.

In certain embodiments, the patient has been administered a prior treatment for uterine cancer, such as cisplatin, doxorubicin, liposomal doxorubicin, docetaxel, trastuzumab, fulvestrant, topotecan, paclitaxel, bevacizumab, carboplatin, temsirolimus tamoxifen, an aromatase inhibitor, and combinations thereof.

In the methods of treatment described herein, additional therapeutic agents can be used in combination with the therapeutic agents described herein that are suitable to the disease being treated (e.g., cancer). In some aspects, the provided methods of treatment further include administering an additional therapeutic agent to the subject. The additional therapeutic agent may be a drug, compound, inhibitor, modulator, or antagonist, having the ability to inhibit the growth or proliferation of cells and/or having antineoplastic properties. In some aspects, the additional therapeutic agent can be a chemotherapeutic agent. In further aspects, the additional therapeutic agent is a therapeutic agent approved by the FDA or an equivalent or similar regulatory agency, for treating cancer. In certain aspects, the additional therapeutic agent is radiation therapy. Suitable additional therapeutic agents include, but are not limited to anesthetics, anticonvulsants, analgesics, allosteric inhibitors, antihelmintics, analeptics, antifungals, antirheumatic agents, anticholinesterases, anticholinergic agents, antibiotics, antineoplastic agents, antimuscarinic agents, growth factors, anabolic steroids, hematological agents, antimycobacterial agents, psychotherapeutic agents, antiviral agents, neural blocking agents, immunological agents, anti-inflammatory agents, protease inhibitors, corticosteroids, anticoagulants, antihistamines, antiprotozoal agents, dopaminergics, muscarinics, vitamins, and hormones. The choice of agent and dosage can be determined by one of skill in the art based on several factors, primarily the given disease being treated.

In some embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, or pharmaceutical composition thereof, is administered as a weight-based dose to a subject.

In some embodiments, the SHP2 inhibitor and/or the binding partner is administered as a weight-based dose to a subject.

A “weight-based dose” (e.g., a dose measured in mg/kg) as used herein, is a dose of the SHP2 inhibitor, the binding partner, and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 that will vary depending on the subject's weight.

In other embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, MP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, is administered as a fixed dose.

In other embodiments, the SHP2 inhibitor and/or the binding partner is administered as a fixed dose.

A “fixed dose” (e.g., a dose measured in mg) means that one dose of the SHP2 inhibitor, the binding partner, and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, is used consistently for all subjects regardless of any subject-specific factors, e.g., weight.

In some embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered at a dose of about 0.05 mg/kg to about 1 g/kg, about 0.1 mg/kg to about 1 g/kg, about 0.5 mg/kg to about 50 mg/kg, about 0.5 mg/kg to about 25 mg/kg, about 0.5 mg/kg to about 10 mg/kg, about 1.0 mg/kg to about 10 mg/kg, about 1.0 mg/kg to about 5 mg/kg, about 1.0 mg/kg to about 2 mg/kg, about 0.1 mg/kg to about 5 mg/kg, about 0.1 mg/kg to about 1 mg/kg, about 0.1 mg/kg to about 0.75 mg/kg, about 0.1 mg/kg to about 0.5 mg/kg, about 1.25 mg/kg to about 4 mg/kg, about 1.0 mg/kg, about 1.5 mg/kg, about 2.0 mg/kg, about 2.5 mg/kg, about 3.0 mg/kg, about 4.0 mg/kg, or about 5.0 mg/kg.

In some embodiments, the SHP2 inhibitor and/or the binding partner is administered at a dose of about 0.05 mg/kg to about 1 g/kg, about 0.1 mg/kg to about 1 g/kg, about 0.5 mg/kg to about 50 mg/kg, about 0.5 mg/kg to about 25 mg/kg, about 0.5 mg/kg to about 10 mg/kg, about 1.0 mg/kg to about 10 mg/kg, about 1.0 mg/kg to about 5 mg/kg, about 1.0 mg/kg to about 2 mg/kg, about 0.1 mg/kg to about 5 mg/kg, about 0.1 mg/kg to about 1 mg/kg, about 0.1 mg/kg to about 0.75 mg/kg, about 0.1 mg/kg to about 0.5 mg/kg, about 1.25 mg/kg to about 4 mg/kg, about 1.0 mg/kg, about 1.5 mg/kg, about 2.0 mg/kg, about 2.5 mg/kg, about 3.0 mg/kg, about 4.0 mg/kg, or about 5.0 mg/kg.

In some embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered at a dose of about 10 mg to about 100 mg, about 10 mg to about 98 mg, about 10 mg to about 95 mg, about 10 mg to about 92 mg, about 10 mg to about 90 mg, about 10 mg to about 88 mg, about 10 mg to about 85 mg, about 10 mg to about 82 mg, about 10 mg to about 80 mg, about 10 mg to about 78 mg, about 10 mg to about 75 mg, about 10 mg to about 72 mg, about 10 mg to about 70 mg, about 10 mg to about 68 mg, about 10 mg to about 65 mg, about 10 mg to about 62 mg, about 10 mg to about 60 mg, about 10 mg to about 58 mg, about 10 mg to about 55 mg, about 10 mg to about 52 mg, about 10 mg to about 50 mg, about 10 mg to about 48 mg, about 10 mg to about 45 mg, about 10 mg to about 42 mg, about 10 mg to about 40 mg, about 10 mg to about 38 mg, about 10 mg to about 35 mg, about 10 mg to about 32 mg, about 10 mg to about 30 mg, about 10 mg to about 28 mg, about 10 mg to about 25 mg, about 10 mg to about 22 mg, about 10 mg to about 20 mg, about 10 mg to about 18 mg, about 10 mg to about 15 mg, about 10 mg to about 12 mg, about 15 mg to about 100 mg, about 15 mg to about 98 mg, about 15 mg to about 95 mg, about 15 mg to about 92 mg, about 15 mg to about 90 mg, about 15 mg to about 88 mg, about 15 mg to about 85 mg, about 15 mg to about 82 mg, about 15 mg to about 80 mg, about 15 mg to about 78 mg, about 15 mg to about 75 mg, about 15 mg to about 72 mg, about 15 mg to about 70 mg, about 15 mg to about 68 mg, about 15 mg to about 65 mg, about 15 mg to about 62 mg, about 15 mg to about 60 mg, about 15 mg to about 58 mg, about 15 mg to about 55 mg, about 15 mg to about 52 mg, about 15 mg to about 50 mg, about 15 mg to about 48 mg, about 15 mg to about 45 mg, about 15 mg to about 42 mg, about 15 mg to about 40 mg, about 15 mg to about 38 mg, about 15 mg to about 35 mg, about 15 mg to about 32 mg, about 15 mg to about 30 mg, about 15 mg to about 28 mg, about 15 mg to about 25 mg, about 15 mg to about 22 mg, about 15 mg to about 20 mg, about 15 mg to about 18 mg, about 20 mg to about 100 mg, about 20 mg to about 98 mg, about 20 mg to about 95 mg, about 20 mg to about 92 mg, about 20 mg to about 90 mg, about 20 mg to about 88 mg, about 20 mg to about 85 mg, about 20 mg to about 82 mg, about 20 mg to about 80 mg, about 20 mg to about 78 mg, about 20 mg to about 75 mg, about 20 mg to about 72 mg, about 20 mg to about 70 mg, about 20 mg to about 68 mg, about 20 mg to about 65 mg, about 20 mg to about 62 mg, about 20 mg to about 60 mg, about 20 mg to about 58 mg, about 20 mg to about 55 mg, about 20 mg to about 52 mg, about 20 mg to about 50 mg, about 20 mg to about 48 mg, about 20 mg to about 45 mg, about 20 mg to about 42 mg, about 20 mg to about 40 mg, about 20 mg to about 38 mg, about 20 mg to about 35 mg, about 20 mg to about 32 mg, about 20 mg to about 30 mg, about 20 mg to about 28 mg, about 20 mg to about 25 mg, about 20 mg to about 22 mg, about 25 mg to about 100 mg, about 25 mg to about 98 mg, about 25 mg to about 95 mg, about 25 mg to about 92 mg, about 25 mg to about 90 mg, about 25 mg to about 88 mg, about 25 mg to about 85 mg, about 25 mg to about 82 mg, about 25 mg to about 80 mg, about 25 mg to about 78 mg, about 25 mg to about 75 mg, about 25 mg to about 72 mg, about 25 mg to about 70 mg, about 25 mg to about 68 mg, about 25 mg to about 65 mg, about 25 mg to about 62 mg, about 25 mg to about 60 mg, about 25 mg to about 58 mg, about 25 mg to about 55 mg, about 25 mg to about 52 mg, about 25 mg to about 50 mg, about 25 mg to about 48 mg, about 25 mg to about 45 mg, about 25 mg to about 42 mg, about 25 mg to about 40 mg, about 25 mg to about 38 mg, about 25 mg to about 35 mg, about 25 mg to about 32 mg, about 25 mg to about 30 mg, about 25 mg to about 28 mg, about 30 mg to about 100 mg, about 30 mg to about 98 mg, about 30 mg to about 95 mg, about 30 mg to about 92 mg, about 30 mg to about 90 mg, about 30 mg to about 88 mg, about 30 mg to about 85 mg, about 30 mg to about 82 mg, about 30 mg to about 80 mg, about 30 mg to about 78 mg, about 30 mg to about 75 mg, about 30 mg to about 72 mg, about 30 mg to about 70 mg, about 30 mg to about 68 mg, about 30 mg to about 65 mg, about 30 mg to about 62 mg, about 30 mg to about 60 mg, about 30 mg to about 58 mg, about 30 mg to about 55 mg, about 30 mg to about 52 mg, about 30 mg to about 50 mg, about 30 mg to about 48 mg, about 30 mg to about 45 mg, about 30 mg to about 42 mg, about 30 mg to about 40 mg, about 30 mg to about 38 mg, about 30 mg to about 35 mg, about 30 mg to about 32 mg, about 35 mg to about 100 mg, about 35 mg to about 98 mg, about 35 mg to about 95 mg, about 35 mg to about 92 mg, about 35 mg to about 90 mg, about 35 mg to about 88 mg, about 35 mg to about 85 mg, about 35 mg to about 82 mg, about 35 mg to about 80 mg, about 35 mg to about 78 mg, about 35 mg to about 75 mg, about 35 mg to about 72 mg, about 35 mg to about 70 mg, about 35 mg to about 68 mg, about 35 mg to about 65 mg, about 35 mg to about 62 mg, about 35 mg to about 60 mg, about 35 mg to about 58 mg, about 35 mg to about 55 mg, about 35 mg to about 52 mg, about 35 mg to about 50 mg, about 35 mg to about 48 mg, about 35 mg to about 45 mg, about 35 mg to about 42 mg, about 35 mg to about 40 mg, about 35 mg to about 38 mg, about 40 mg to about 100 mg, about 40 mg to about 98 mg, about 40 mg to about 95 mg, about 40 mg to about 92 mg, about 40 mg to about 90 mg, about 40 mg to about 88 mg, about 40 mg to about 85 mg, about 40 mg to about 82 mg, about 40 mg to about 80 mg, about 40 mg to about 78 mg, about 40 mg to about 75 mg, about 40 mg to about 72 mg, about 40 mg to about 70 mg, about 40 mg to about 68 mg, about 40 mg to about 65 mg, about 40 mg to about 62 mg, about 40 mg to about 60 mg, about 40 mg to about 58 mg, about 40 mg to about 55 mg, about 40 mg to about 52 mg, about 40 mg to about 50 mg, about 40 mg to about 48 mg, about 40 mg to about 45 mg, about 40 mg to about 42 mg, about 45 mg to about 100 mg, about 45 mg to about 98 mg, about 45 mg to about 95 mg, about 45 mg to about 92 mg, about 45 mg to about 90 mg, about 45 mg to about 88 mg, about 45 mg to about 85 mg, about 45 mg to about 82 mg, about 45 mg to about 80 mg, about 45 mg to about 78 mg, about 45 mg to about 75 mg, about 45 mg to about 72 mg, about 45 mg to about 70 mg, about 45 mg to about 68 mg, about 45 mg to about 65 mg, about 45 mg to about 62 mg, about 45 mg to about 60 mg, about 45 mg to about 58 mg, about 45 mg to about 55 mg, about 45 mg to about 52 mg, about 45 mg to about 50 mg, about 45 mg to about 48 mg, about 50 mg to about 100 mg, about 50 mg to about 98 mg, about 50 mg to about 95 mg, about 50 mg to about 92 mg, about 50 mg to about 90 mg, about 50 mg to about 88 mg, about 50 mg to about 85 mg, about 50 mg to about 82 mg, about 50 mg to about 80 mg, about 50 mg to about 78 mg, about 50 mg to about 75 mg, about 50 mg to about 72 mg, about 50 mg to about 70 mg, about 50 mg to about 68 mg, about 50 mg to about 65 mg, about 50 mg to about 62 mg, about 50 mg to about 60 mg, about 50 mg to about 58 mg, about 50 mg to about 55 mg, about 50 mg to about 52 mg, about 55 mg to about 100 mg, about 55 mg to about 98 mg, about 55 mg to about 95 mg, about 55 mg to about 92 mg, about 55 mg to about 90 mg, about 55 mg to about 88 mg, about 55 mg to about 85 mg, about 55 mg to about 82 mg, about 55 mg to about 80 mg, about 55 mg to about 78 mg, about 55 mg to about 75 mg, about 55 mg to about 72 mg, about 55 mg to about 70 mg, about 55 mg to about 68 mg, about 55 mg to about 65 mg, about 55 mg to about 62 mg, about 55 mg to about 60 mg, about 55 mg to about 58 mg, about 60 mg to about 100 mg, about 60 mg to about 98 mg, about 60 mg to about 95 mg, about 60 mg to about 92 mg, about 60 mg to about 90 mg, about 60 mg to about 88 mg, about 60 mg to about 85 mg, about 60 mg to about 82 mg, about 60 mg to about 80 mg, about 60 mg to about 78 mg, about 60 mg to about 75 mg, about 60 mg to about 72 mg, about 60 mg to about 70 mg, about 60 mg to about 68 mg, about 60 mg to about 65 mg, about 60 mg to about 62 mg, about 65 mg to about 100 mg, about 65 mg to about 98 mg, about 65 mg to about 95 mg, about 65 mg to about 92 mg, about 65 mg to about 90 mg, about 65 mg to about 88 mg, about 65 mg to about 85 mg, about 65 mg to about 82 mg, about 65 mg to about 80 mg, about 65 mg to about 82 mg, about 65 mg to about 75 mg, about 65 mg to about 72 mg, about 65 mg to about 70 mg, about 65 mg to about 68 mg, about 70 mg to about 100 mg, about 70 mg to about 98 mg, about 70 mg to about 95 mg, about 70 mg to about 92 mg, about 70 mg to about 90 mg, about 70 mg to about 88 mg, about 70 mg to about 85 mg, about 70 mg to about 82 mg, about 70 mg to about 80 mg, about 70 mg to about 78 mg, about 70 mg to about 75 mg, about 70 mg to about 72 mg, about 75 mg to about 100 mg, about 75 mg to about 98 mg, about 75 mg to about 95 mg, about 75 mg to about 92 mg, about 75 mg to about 90 mg, about 75 mg to about 88 mg, about 75 mg to about 85 mg, about 75 mg to about 82 mg, about 75 mg to about 80 mg, about 75 mg to about 78 mg, about 80 mg to about 100 mg, about 80 mg to about 98 mg, about 80 mg to about 95 mg, about 80 mg to about 92 mg, about 80 mg to about 90 mg, about 80 mg to about 88 mg, about 80 mg to about 85 mg, about 80 mg to about 82 mg, about 85 mg to about 100 mg, about 85 mg to about 98 mg, about 85 mg to about 95 mg, about 85 mg to about 92 mg, about 85 mg to about 90 mg, about 85 mg to about 88 mg, about 90 mg to about 100 mg, about 90 mg to about 98 mg, about 90 mg to about 95 mg, about 90 mg to about 92 mg, about 95 mg to about 100 mg, about 95 mg to about 98 mg, about 10 mg, about 12 mg, about 15 mg, about 18 mg, about 20 mg, about 22 mg, about 25 mg, about 28 mg, about 30 mg, about 32 mg, about 35 mg, about 38 mg, about 40 mg, about 42 mg, about 45 mg, about 48 mg, about 50 mg, about 52 mg, about 55 mg, about 58 mg, about 60 mg, about 62 mg, about 65 mg, about 68 mg, about 70 mg, about 72 mg, about 75 mg, about 78 mg, about 80 mg, about 82 mg, about 85 mg, about 88 mg, about 90 mg, about 92 mg, about 95 mg, about 98 mg, or about 100 mg.

In some embodiments, the SHP2 inhibitor and/or the binding partner is administered at a dose of about 10 mg to about 100 mg, about 10 mg to about 98 mg, about 10 mg to about 95 mg, about 10 mg to about 92 mg, about 10 mg to about 90 mg, about 10 mg to about 88 mg, about 10 mg to about 85 mg, about 10 mg to about 82 mg, about 10 mg to about 80 mg, about 10 mg to about 78 mg, about 10 mg to about 75 mg, about 10 mg to about 72 mg, about 10 mg to about 70 mg, about 10 mg to about 68 mg, about 10 mg to about 65 mg, about 10 mg to about 62 mg, about 10 mg to about 60 mg, about 10 mg to about 58 mg, about 10 mg to about 55 mg, about 10 mg to about 52 mg, about 10 mg to about 50 mg, about 10 mg to about 48 mg, about 10 mg to about 45 mg, about 10 mg to about 42 mg, about 10 mg to about 40 mg, about 10 mg to about 38 mg, about 10 mg to about 35 mg, about 10 mg to about 32 mg, about 10 mg to about 30 mg, about 10 mg to about 28 mg, about 10 mg to about 25 mg, about 10 mg to about 22 mg, about 10 mg to about 20 mg, about 10 mg to about 18 mg, about 10 mg to about 15 mg, about 10 mg to about 12 mg, about 15 mg to about 100 mg, about 15 mg to about 98 mg, about 15 mg to about 95 mg, about 15 mg to about 92 mg, about 15 mg to about 90 mg, about 15 mg to about 88 mg, about 15 mg to about 85 mg, about 15 mg to about 82 mg, about 15 mg to about 80 mg, about 15 mg to about 78 mg, about 15 mg to about 75 mg, about 15 mg to about 72 mg, about 15 mg to about 70 mg, about 15 mg to about 68 mg, about 15 mg to about 65 mg, about 15 mg to about 62 mg, about 15 mg to about 60 mg, about 15 mg to about 58 mg, about 15 mg to about 55 mg, about 15 mg to about 52 mg, about 15 mg to about 50 mg, about 15 mg to about 48 mg, about 15 mg to about 45 mg, about 15 mg to about 42 mg, about 15 mg to about 40 mg, about 15 mg to about 38 mg, about 15 mg to about 35 mg, about 15 mg to about 32 mg, about 15 mg to about 30 mg, about 15 mg to about 28 mg, about 15 mg to about 25 mg, about 15 mg to about 22 mg, about 15 mg to about 20 mg, about 15 mg to about 18 mg, about 20 mg to about 100 mg, about 20 mg to about 98 mg, about 20 mg to about 95 mg, about 20 mg to about 92 mg, about 20 mg to about 90 mg, about 20 mg to about 88 mg, about 20 mg to about 85 mg, about 20 mg to about 82 mg, about 20 mg to about 80 mg, about 20 mg to about 78 mg, about 20 mg to about 75 mg, about 20 mg to about 72 mg, about 20 mg to about 70 mg, about 20 mg to about 68 mg, about 20 mg to about 65 mg, about 20 mg to about 62 mg, about 20 mg to about 60 mg, about 20 mg to about 58 mg, about 20 mg to about 55 mg, about 20 mg to about 52 mg, about 20 mg to about 50 mg, about 20 mg to about 48 mg, about 20 mg to about 45 mg, about 20 mg to about 42 mg, about 20 mg to about 40 mg, about 20 mg to about 38 mg, about 20 mg to about 35 mg, about 20 mg to about 32 mg, about 20 mg to about 30 mg, about 20 mg to about 28 mg, about 20 mg to about 25 mg, about 20 mg to about 22 mg, about 25 mg to about 100 mg, about 25 mg to about 98 mg, about 25 mg to about 95 mg, about 25 mg to about 92 mg, about 25 mg to about 90 mg, about 25 mg to about 88 mg, about 25 mg to about 85 mg, about 25 mg to about 82 mg, about 25 mg to about 80 mg, about 25 mg to about 78 mg, about 25 mg to about 75 mg, about 25 mg to about 72 mg, about 25 mg to about 70 mg, about 25 mg to about 68 mg, about 25 mg to about 65 mg, about 25 mg to about 62 mg, about 25 mg to about 60 mg, about 25 mg to about 58 mg, about 25 mg to about 55 mg, about 25 mg to about 52 mg, about 25 mg to about 50 mg, about 25 mg to about 48 mg, about 25 mg to about 45 mg, about 25 mg to about 42 mg, about 25 mg to about 40 mg, about 25 mg to about 38 mg, about 25 mg to about 35 mg, about 25 mg to about 32 mg, about 25 mg to about 30 mg, about 25 mg to about 28 mg, about 30 mg to about 100 mg, about 30 mg to about 98 mg, about 30 mg to about 95 mg, about 30 mg to about 92 mg, about 30 mg to about 90 mg, about 30 mg to about 88 mg, about 30 mg to about 85 mg, about 30 mg to about 82 mg, about 30 mg to about 80 mg, about 30 mg to about 78 mg, about 30 mg to about 75 mg, about 30 mg to about 72 mg, about 30 mg to about 70 mg, about 30 mg to about 68 mg, about 30 mg to about 65 mg, about 30 mg to about 62 mg, about 30 mg to about 60 mg, about 30 mg to about 58 mg, about 30 mg to about 55 mg, about 30 mg to about 52 mg, about 30 mg to about 50 mg, about 30 mg to about 48 mg, about 30 mg to about 45 mg, about 30 mg to about 42 mg, about 30 mg to about 40 mg, about 30 mg to about 38 mg, about 30 mg to about 35 mg, about 30 mg to about 32 mg, about 35 mg to about 100 mg, about 35 mg to about 98 mg, about 35 mg to about 95 mg, about 35 mg to about 92 mg, about 35 mg to about 90 mg, about 35 mg to about 88 mg, about 35 mg to about 85 mg, about 35 mg to about 82 mg, about 35 mg to about 80 mg, about 35 mg to about 78 mg, about 35 mg to about 75 mg, about 35 mg to about 72 mg, about 35 mg to about 70 mg, about 35 mg to about 68 mg, about 35 mg to about 65 mg, about 35 mg to about 62 mg, about 35 mg to about 60 mg, about 35 mg to about 58 mg, about 35 mg to about 55 mg, about 35 mg to about 52 mg, about 35 mg to about 50 mg, about 35 mg to about 48 mg, about 35 mg to about 45 mg, about 35 mg to about 42 mg, about 35 mg to about 40 mg, about 35 mg to about 38 mg, about 40 mg to about 100 mg, about 40 mg to about 98 mg, about 40 mg to about 95 mg, about 40 mg to about 92 mg, about 40 mg to about 90 mg, about 40 mg to about 88 mg, about 40 mg to about 85 mg, about 40 mg to about 82 mg, about 40 mg to about 80 mg, about 40 mg to about 78 mg, about 40 mg to about 75 mg, about 40 mg to about 72 mg, about 40 mg to about 70 mg, about 40 mg to about 68 mg, about 40 mg to about 65 mg, about 40 mg to about 62 mg, about 40 mg to about 60 mg, about 40 mg to about 58 mg, about 40 mg to about 55 mg, about 40 mg to about 52 mg, about 40 mg to about 50 mg, about 40 mg to about 48 mg, about 40 mg to about 45 mg, about 40 mg to about 42 mg, about 45 mg to about 100 mg, about 45 mg to about 98 mg, about 45 mg to about 95 mg, about 45 mg to about 92 mg, about 45 mg to about 90 mg, about 45 mg to about 88 mg, about 45 mg to about 85 mg, about 45 mg to about 82 mg, about 45 mg to about 80 mg, about 45 mg to about 78 mg, about 45 mg to about 75 mg, about 45 mg to about 72 mg, about 45 mg to about 70 mg, about 45 mg to about 68 mg, about 45 mg to about 65 mg, about 45 mg to about 62 mg, about 45 mg to about 60 mg, about 45 mg to about 58 mg, about 45 mg to about 55 mg, about 45 mg to about 52 mg, about 45 mg to about 50 mg, about 45 mg to about 48 mg, about 50 mg to about 100 mg, about 50 mg to about 98 mg, about 50 mg to about 95 mg, about 50 mg to about 92 mg, about 50 mg to about 90 mg, about 50 mg to about 88 mg, about 50 mg to about 85 mg, about 50 mg to about 82 mg, about 50 mg to about 80 mg, about 50 mg to about 78 mg, about 50 mg to about 75 mg, about 50 mg to about 72 mg, about 50 mg to about 70 mg, about 50 mg to about 68 mg, about 50 mg to about 65 mg, about 50 mg to about 62 mg, about 50 mg to about 60 mg, about 50 mg to about 58 mg, about 50 mg to about 55 mg, about 50 mg to about 52 mg, about 55 mg to about 100 mg, about 55 mg to about 98 mg, about 55 mg to about 95 mg, about 55 mg to about 92 mg, about 55 mg to about 90 mg, about 55 mg to about 88 mg, about 55 mg to about 85 mg, about 55 mg to about 82 mg, about 55 mg to about 80 mg, about 55 mg to about 78 mg, about 55 mg to about 75 mg, about 55 mg to about 72 mg, about 55 mg to about 70 mg, about 55 mg to about 68 mg, about 55 mg to about 65 mg, about 55 mg to about 62 mg, about 55 mg to about 60 mg, about 55 mg to about 58 mg, about 60 mg to about 100 mg, about 60 mg to about 98 mg, about 60 mg to about 95 mg, about 60 mg to about 92 mg, about 60 mg to about 90 mg, about 60 mg to about 88 mg, about 60 mg to about 85 mg, about 60 mg to about 82 mg, about 60 mg to about 80 mg, about 60 mg to about 78 mg, about 60 mg to about 75 mg, about 60 mg to about 72 mg, about 60 mg to about 70 mg, about 60 mg to about 68 mg, about 60 mg to about 65 mg, about 60 mg to about 62 mg, about 65 mg to about 100 mg, about 65 mg to about 98 mg, about 65 mg to about 95 mg, about 65 mg to about 92 mg, about 65 mg to about 90 mg, about 65 mg to about 88 mg, about 65 mg to about 85 mg, about 65 mg to about 82 mg, about 65 mg to about 80 mg, about 65 mg to about 82 mg, about 65 mg to about 75 mg, about 65 mg to about 72 mg, about 65 mg to about 70 mg, about 65 mg to about 68 mg, about 70 mg to about 100 mg, about 70 mg to about 98 mg, about 70 mg to about 95 mg, about 70 mg to about 92 mg, about 70 mg to about 90 mg, about 70 mg to about 88 mg, about 70 mg to about 85 mg, about 70 mg to about 82 mg, about 70 mg to about 80 mg, about 70 mg to about 78 mg, about 70 mg to about 75 mg, about 70 mg to about 72 mg, about 75 mg to about 100 mg, about 75 mg to about 98 mg, about 75 mg to about 95 mg, about 75 mg to about 92 mg, about 75 mg to about 90 mg, about 75 mg to about 88 mg, about 75 mg to about 85 mg, about 75 mg to about 82 mg, about 75 mg to about 80 mg, about 75 mg to about 78 mg, about 80 mg to about 100 mg, about 80 mg to about 98 mg, about 80 mg to about 95 mg, about 80 mg to about 92 mg, about 80 mg to about 90 mg, about 80 mg to about 88 mg, about 80 mg to about 85 mg, about 80 mg to about 82 mg, about 85 mg to about 100 mg, about 85 mg to about 98 mg, about 85 mg to about 95 mg, about 85 mg to about 92 mg, about 85 mg to about 90 mg, about 85 mg to about 88 mg, about 90 mg to about 100 mg, about 90 mg to about 98 mg, about 90 mg to about 95 mg, about 90 mg to about 92 mg, about 95 mg to about 100 mg, about 95 mg to about 98 mg, about 10 mg, about 12 mg, about 15 mg, about 18 mg, about 20 mg, about 22 mg, about 25 mg, about 28 mg, about 30 mg, about 32 mg, about 35 mg, about 38 mg, about 40 mg, about 42 mg, about 45 mg, about 48 mg, about 50 mg, about 52 mg, about 55 mg, about 58 mg, about 60 mg, about 62 mg, about 65 mg, about 68 mg, about 70 mg, about 72 mg, about 75 mg, about 78 mg, about 80 mg, about 82 mg, about 85 mg, about 88 mg, about 90 mg, about 92 mg, about 95 mg, about 98 mg, or about 100 mg.

In certain embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered to a subject at a dosing frequency of about five times a week, about four times a week, about twice a week, about once a week, about once every two weeks, about once every three weeks, about once a month, about once every five weeks, about once every six weeks, about once every seven weeks, about once every two months, about once every three months, or less frequently so long as an effective therapeutic response is achieved. In certain embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 can be administered to a subject at a dosing frequency of about six times a year, about four times a year, about twice a year, about once a year, about once every two years, about once every three years, about once every four years, about once every five years, about once every six years, about once every eight years, about once a decade, about once every twelve years, about once every fifteen years, or less frequently so long as an effective therapeutic response is achieved.

In certain embodiments, the SHP2 inhibitor and/or the binding partner is administered to a subject at a dosing frequency of about five times a week, about four times a week, about twice a week, about once a week, about once every two weeks, about once every three weeks, about once a month, about once every five weeks, about once every six weeks, about once every seven weeks, about once every two months, about once every three months, or less frequently so long as an effective therapeutic response is achieved. In certain embodiments, the SHP2 inhibitor and/or the binding partner can be administered to a subject at a dosing frequency of about six times a year, about four times a year, about twice a year, about once a year, about once every two years, about once every three years, about once every four years, about once every five years, about once every six years, about once every eight years, about once a decade, about once every twelve years, about once every fifteen years, or less frequently so long as an effective therapeutic response is achieved.

Dosage ranges and frequency of administration of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 as described herein can vary depending on the parameters of a specific subject, the route of administration, the type of medical condition, and/or the severity of the medical condition. A more accurate dose may depend on the subject in which it is administered, e.g., a lower dose may be used if the subject in which it is administered is juvenile and a higher dose may be used if the subject is adult. In some embodiments, a more accurate dose may depend on the subject weight.

Dosage ranges and frequency of administration of an SHP2 inhibitor and/or a binding partner as described herein can vary depending on the parameters of a specific subject, the route of administration, the type of medical condition, and/or the severity of the medical condition. A more accurate dose may depend on the subject in which it is administered, e.g., a lower dose may be used if the subject in which it is administered is juvenile and a higher dose may be used if the subject is adult. In some embodiments, a more accurate dose may depend on the subject weight.

In certain embodiments, multiple doses of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are administered over a defined time course.

In certain embodiments, multiple doses of an SHP2 inhibitor and/or a binding partner are administered over a defined time course.

In some embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, MP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered following a repeated dosing regimen wherein the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered an initial time (e.g., as an initial dose) and then may be re-administered at any amount for any number of subsequent times thereafter over the time course of treatment. For example, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be re-administered one time, two times, three times, four times, five times, six times, seven times, eight times, nine times, ten times, fifteen times, twenty times, or more, over the time course of treatment which may occur over any amount of time (e.g., days, weeks, or years). In certain embodiments, when the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, the inhibitor(s) which is/are administered first in a repeat dosing regimen may comprise the same inhibitor(s) which is/are re-administered second or thereafter in the regimen, for any number of subsequent times thereafter. In certain embodiments, when the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, the inhibitor(s) which is/are administered first in a repeat dosing regimen may comprise a different inhibitor(s) than is/are re-administered second or thereafter in the regimen, for any number of subsequent times thereafter.

In some embodiments, the SHP2 inhibitor and/or the binding partner may be administered following a repeated dosing regimen wherein the SHP2 inhibitor and/or the binding partner may be administered an initial time (e.g., as an initial dose) and then may be re-administered at any amount for any number of subsequent times thereafter over the time course of treatment. For example, the SHP2 inhibitor and/or the binding partner may be re-administered one time, two times, three times, four times, five times, six times, seven times, eight times, nine times, ten times, fifteen times, twenty times, or more, over the time course of treatment which may occur over any amount of time (e.g., days, weeks, or years). In certain embodiments, when the SHP2 inhibitor and/or the binding partner, the inhibitor/binding partner which is/are administered first in a repeat dosing regimen may comprise the same inhibitor/binding partner which is/are re-administered second or thereafter in the regimen, for any number of subsequent times thereafter. In certain embodiments, when the SHP2 inhibitor and/or the binding partner, the inhibitor/binding partner which is/are administered first in a repeat dosing regimen may comprise a different inhibitor/binding partner than is/are re-administered second or thereafter in the regimen, for any number of subsequent times thereafter.

In certain embodiments, the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, may be administered using a stepwise dosing regimen. Stepwise dosing can refer to dividing dosing of the same inhibitor(s) over multiple administrations. In certain embodiments, the dosing of the same inhibitor(s) is/are broken up one time, two times, three times, four times, five times, six times, seven times, eight times, nine times, ten times, fifteen times, twenty times, or more, over the time course of the treatment which can occur over any period of time (e.g., over any number of days, weeks, or years). In certain embodiments, when a stepwise dose regimen is used in the administration of the inhibitor(s), the regimen may encompass an increase or decrease in dosage levels with each administration of the inhibitor(s).

In certain embodiments, the SHP2 inhibitor and/or the binding partner may be administered using a stepwise dosing regimen. Stepwise dosing can refer to dividing dosing of the same inhibitor(s) over multiple administrations. In certain embodiments, the dosing of the same inhibitor(s) is/are broken up one time, two times, three times, four times, five times, six times, seven times, eight times, nine times, ten times, fifteen times, twenty times, or more, over the time course of the treatment which can occur over any period of time (e.g., over any number of days, weeks, or years). In certain embodiments, when a stepwise dose regimen is used in the administration of the inhibitor(s), the regimen may encompass an increase or decrease in dosage levels with each administration of the inhibitor(s).

In some embodiments, the SHP2 inhibitor and the inhibitor of expression or function or degrader of said one or more proteins are administered simultaneously.

In some embodiments, the SHP2 inhibitor and the binding partner are administered simultaneously.

In some embodiments, the SHP2 inhibitor and the inhibitor of expression or function or degrader of said one or more proteins are administered simultaneously in one composition.

In some embodiments, the SHP2 inhibitor and the binding partner are administered simultaneously in one composition.

In some embodiments, the SHP2 inhibitor and the inhibitor of expression or function or degrader of said one or more proteins are administered simultaneously in different compositions.

In some embodiments, the SHP2 inhibitor and the binding partner are administered simultaneously in different compositions.

In some embodiments, the SHP2 inhibitor and the inhibitor of expression or function or degrader of said one or more proteins are administered sequentially.

In some embodiments, the SHP2 inhibitor and the binding partner are administered sequentially.

In certain embodiments, the SHP2 inhibitor is administered to a subject in combination with an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. The SHP2 inhibitor can be administered prior to, simultaneously with, and/or after the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the SHP2 inhibitor is administered to a subject in combination with a binding partner. The SHP2 inhibitor can be administered prior to, simultaneously with, and/or after the binding partner.

In certain embodiments, when the SHP2 inhibitor is administered prior to, simultaneously with, and/or after the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, the SHP2 inhibitor is administered one time, two times, three times, four times, five times, six times, seven times, eight times, nine times, ten times, twelve times, fourteen times, fifteen times, eighteen times, or twenty times or more, prior to, simultaneously with, and/or after the administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In certain embodiments, when an SHP2 inhibitor is administered with a repeat dosing regimen, an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered once, twice, or any number of times prior to, simultaneously with, and/or after a first, second, and/or any number of subsequent administrations of the SHP2 inhibitor thereafter.

In certain embodiments, when the SHP2 inhibitor is administered prior to, simultaneously with, and/or after the binding partner, the SHP2 inhibitor is administered one time, two times, three times, four times, five times, six times, seven times, eight times, nine times, ten times, twelve times, fourteen times, fifteen times, eighteen times, or twenty times or more, prior to, simultaneously with, and/or after the administration of the binding partner. In certain embodiments, when an SHP2 inhibitor is administered with a repeat dosing regimen, a binding partner may be administered once, twice, or any number of times prior to, simultaneously with, and/or after a first, second, and/or any number of subsequent administrations of the SHP2 inhibitor thereafter.

In some embodiments, an SHP2 inhibitor may be administered to a subject separately from an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, an SHP2 inhibitor may be administered to a subject separately from a binding partner.

In some embodiments, when an SHP2 inhibitor and an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are administered separately, the SHP2 inhibitor may also be administered simultaneously with the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In certain embodiments, the SHP2 inhibitor is administered one or multiple times during the administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In another embodiment, the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered one or multiple times during the administration of the SHP2 inhibitor.

In some embodiments, when an SHP2 inhibitor and a binding partner are administered separately, the SHP2 inhibitor may also be administered simultaneously with the binding partner. In certain embodiments, the SHP2 inhibitor is administered one or multiple times during the administration of the binding partner. In another embodiment, the binding partner is administered one or multiple times during the administration of the SHP2 inhibitor.

In certain embodiments, an SHP2 inhibitor and an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered separately over a defined time period. In some embodiments, multiple doses of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, MP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered to a subject through a defined time course. The methods may comprise sequentially administering to a subject multiple doses of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In certain embodiments, when the SHP2 inhibitor and inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are administered sequentially, the SHP2 inhibitor can be administered either before and/or in between each of the administrations of the inhibitor(s) of expression or function or the degrader(s) of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, an SHP2 inhibitor and a binding partner may be administered separately over a defined time period. In some embodiments, multiple doses of an SHP2 inhibitor and/or a binding partner may be administered to a subject through a defined time course. The methods may comprise sequentially administering to a subject multiple doses of an SHP2 inhibitor and/or a binding partner. In certain embodiments, when the SHP2 inhibitor and binding partner are administered sequentially, the SHP2 inhibitor can be administered either before and/or in between each of the administrations of the binding partner.

The term “sequentially administering”, as used herein, means that each dose of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, is administered to the subject at various points in time, e.g., on different days separated by an interval that may comprise hours, days, weeks, months, or years. In certain embodiments, the methods comprise sequentially administering to the subject a single initial dose of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, followed by one or multiple secondary doses of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, and optionally followed by one or multiple tertiary doses of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

The term “sequentially administering”, as used herein, means that each dose of the SHP2 inhibitor and/or the binding partner is administered to the subject at various points in time, e.g., on different days separated by an interval that may comprise hours, days, weeks, months, or years. In certain embodiments, the methods comprise sequentially administering to the subject a single initial dose of the SHP2 inhibitor and/or the binding partner, followed by one or multiple secondary doses of the SHP2 inhibitor and/or the binding partner, and optionally followed by one or multiple tertiary doses of the SHP2 inhibitor and/or the binding partner.

As used herein, the terms “initial dose,” “secondary dose(s),” and “tertiary dose(s)” refer to the sequential sequence of administration of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. Thus, the “initial dose” is the dose administered at the beginning of the treatment regimen schedule (i.e., the “loading dose”), the “secondary dose(s)” is/are the dose(s) which are administered after the “initial dose”, and the “tertiary dose(s)” is/are the dose(s) which are administered after the “secondary dose(s)”.

As used herein, the terms “initial dose,” “secondary dose(s),” and “tertiary dose(s)” refer to the sequential sequence of administration of the SHP2 inhibitor and/or the binding partner. Thus, the “initial dose” is the dose administered at the beginning of the treatment regimen schedule (i.e., the “loading dose”), the “secondary dose(s)” is/are the dose(s) which are administered after the “initial dose”, and the “tertiary dose(s)” is/are the dose(s) which are administered after the “secondary dose(s)”.

In certain embodiments, the initial, secondary, and tertiary doses may all contain the same amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 but may differ from one another in the frequency of administration. In another embodiment, the amounts of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 contained in the initial, secondary, and/or tertiary doses varies from one another (e.g., increased or decreased as appropriate) during the course of treatment. In some embodiments, one or multiple (e.g., 1, 2, 3, 4, 5, 6, 7, 8, 9, 10, or more) doses are administered in the beginning of the treatment regimen as loading doses (i.e., initial doses) followed by subsequent doses (e.g., secondary or tertiary doses) that are administered less frequently, known as “maintenance doses”. In certain embodiments, the initial dose and the one or more subsequent doses (i.e., secondary or tertiary doses) each contain the same amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In some embodiments, the initial dose comprises an initial amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 and the one or multiple secondary doses each comprise a different amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In one embodiment, the first amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, MP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 can be 1.5×, 2×, 2.5×, 3×, 3.5×, 4×, 4.5×, 5×, 10×, or more than the second amount of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the initial, secondary, and tertiary doses may all contain the same amount of the SHP2 inhibitor and/or the binding partner but may differ from one another in the frequency of administration. In another embodiment, the amounts of the SHP2 inhibitor and/or the binding partner contained in the initial, secondary, and/or tertiary doses varies from one another (e.g., increased or decreased as appropriate) during the course of treatment. In some embodiments, one or multiple (e.g., 1, 2, 3, 4, 5, 6, 7, 8, 9, 10, or more) doses are administered in the beginning of the treatment regimen as loading doses (i.e., initial doses) followed by subsequent doses (e.g., secondary or tertiary doses) that are administered less frequently, known as “maintenance doses”. In certain embodiments, the initial dose and the one or more subsequent doses (i.e., secondary or tertiary doses) each contain the same amount of the SHP2 inhibitor and/or the binding partner. In some embodiments, the initial dose comprises an initial amount of the SHP2 inhibitor and/or the binding partner and the one or multiple secondary doses each comprise a different amount of the SHP2 inhibitor and/or the binding partner. In one embodiment, the first amount of the SHP2 inhibitor and/or the binding partner can be 1.5×, 2×, 2.5×, 3×, 3.5×, 4×, 4.5×, 5×, 10×, or more than the second amount of the SHP2 inhibitor and/or the binding partner.

In certain embodiments, each secondary dose(s) and/or tertiary dose(s) is/are administered 1 to 20 (e.g., 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5 or more) weeks after the immediately preceding dose. As used herein, the phrase “the immediately preceding dose,” means, in a sequence of multiple administrations, the dose of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 that is administered to a subject before the administration of the very next subsequent dose in the sequence with no intervening doses.

In certain embodiments, each secondary dose(s) and/or tertiary dose(s) is/are administered 1 to 20 (e.g., 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5 or more) weeks after the immediately preceding dose. As used herein, the phrase “the immediately preceding dose,” means, in a sequence of multiple administrations, the dose of the SHP2 inhibitor and/or the binding partner that is administered to a subject before the administration of the very next subsequent dose in the sequence with no intervening doses.

The methods may comprise administering to a subject any number of secondary doses and/or tertiary doses of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In some embodiments, only one secondary dose is administered to the subject. In certain embodiments, two or more secondary doses are administered to the subject. Likewise, in some embodiments, only one tertiary dose is administered to the subject. In some embodiments, two or more tertiary doses are administered to the subject.

The methods may comprise administering to a subject any number of secondary doses and/or tertiary doses of an SHP2 inhibitor and/or a binding partner. In some embodiments, only one secondary dose is administered to the subject. In certain embodiments, two or more secondary doses are administered to the subject. Likewise, in some embodiments, only one tertiary dose is administered to the subject. In some embodiments, two or more tertiary doses are administered to the subject.

In certain embodiments that involve multiple secondary doses, all secondary doses are administered at the same frequency as one another (e.g., each secondary dose may be administered 1, 2, 3, or 4 weeks, or some other frequency, after the immediately preceding dose. Similarly, in certain embodiments that involve multiple tertiary doses, all tertiary doses are administered at the same frequency as one another. Alternatively, the frequency at which the secondary doses and/or tertiary doses are administered can vary over the treatment regimen course. The frequency of administration may be adjusted during the treatment regimen course by a physician depending on the subject needs following clinical examination.

In certain embodiments, when an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are sequentially administered, the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be a first component of the dosing regimen and the SHP2 inhibitor may be a second component of the dosing regimen (i.e., the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered before the SHP2 inhibitor). In certain embodiments, the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be a second component of the dosing regimen and the SHP2 inhibitor may be a first component of a dosing regimen (i.e., the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 may be administered after the SHP2 inhibitor). In other embodiments, an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 and an SHP2 inhibitor may be sequentially administered, by using either of the above-described orders with variable time intervals between administrations. For example, the time interval between administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 and the SHP2 inhibitor may be at least about 30 seconds, at least about 45 seconds, at least about 1 minute, at least about 2 minutes, at least about 5 minutes, at least about 10 minutes, at least about 15 minutes, at least about 20 minutes, at least about 30 minutes, at least about 45 minutes, at least about 1 hour, at least about 2 hours, at least about 3 to 4 hours, at least about 5 hours, at least about 6 to 7 hours, at least about 8 to 9 hours, at least about 10 hours, at least about 11 to 12 hours, at least about 13 to 14 hours, at least about 15 hours, at least about 16 to 17 hours, at least about 18 to 19 hours, at least about 20 hours, at least about 21 to 24 hours, at least about 1 day, at least about 2 days, at least about 3 to 4 days, at least about 5 days, at least about 6 to 7 days, at least about 8 to 9 days, at least about 10 days, at least about 11 to 12 days, at least about 13 to 14 days, at least about 15 days, at least about 16 to 17 days, at least about 18 to 19 days, at least about 20 days, at least about 21 to 25 days, at least about 26 to 31 days, at least about 1 month, at least about 2 to 5 months, at least about 6 months, at least about 7 to 11 months, at least about 1 year, at least about 2 to 4 years, at least about 5 years, at least about 6 to 9 years, at least about 10 years, or more.

In certain embodiments, when an SHP2 inhibitor and/or a binding partner are sequentially administered, the binding partner may be a first component of the dosing regimen and the SHP2 inhibitor may be a second component of the dosing regimen (i.e., the binding partner may be administered before the SHP2 inhibitor). In certain embodiments, the binding partner may be a second component of the dosing regimen and the SHP2 inhibitor may be a first component of a dosing regimen (i.e., the binding partner may be administered after the SHP2 inhibitor). In other embodiments, a binding partner and an SHP2 inhibitor may be sequentially administered, by using either of the above-described orders with variable time intervals between administrations. For example, the time interval between administration of the binding partner and the SHP2 inhibitor may be at least about 30 seconds, at least about 45 seconds, at least about 1 minute, at least about 2 minutes, at least about 5 minutes, at least about 10 minutes, at least about 15 minutes, at least about 20 minutes, at least about 30 minutes, at least about 45 minutes, at least about 1 hour, at least about 2 hours, at least about 3 to 4 hours, at least about 5 hours, at least about 6 to 7 hours, at least about 8 to 9 hours, at least about 10 hours, at least about 11 to 12 hours, at least about 13 to 14 hours, at least about 15 hours, at least about 16 to 17 hours, at least about 18 to 19 hours, at least about 20 hours, at least about 21 to 24 hours, at least about 1 day, at least about 2 days, at least about 3 to 4 days, at least about 5 days, at least about 6 to 7 days, at least about 8 to 9 days, at least about 10 days, at least about 11 to 12 days, at least about 13 to 14 days, at least about 15 days, at least about 16 to 17 days, at least about 18 to 19 days, at least about 20 days, at least about 21 to 25 days, at least about 26 to 31 days, at least about 1 month, at least about 2 to 5 months, at least about 6 months, at least about 7 to 11 months, at least about 1 year, at least about 2 to 4 years, at least about 5 years, at least about 6 to 9 years, at least about 10 years, or more.

Any of the methods of administration as described herein can further comprise any of various subsequent administration steps as described herein. For example, the subsequent administration step can comprise administering the SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 to the subject one or more subsequent times until a desired clinical outcome and/or measurement of interest is achieved in the subject.

Any of the methods of administration as described herein can further comprise any of various subsequent administration steps as described herein. For example, the subsequent administration step can comprise administering the SHP2 inhibitor and/or a binding partner to the subject one or more subsequent times until a desired clinical outcome and/or measurement of interest is achieved in the subject.

For example, the subsequent administration step(s) can be at least about 1 week, at least about 2 to 4 weeks, at least about 5 weeks, at least about 6 to 9 weeks, at least about 10 weeks, at least about 11 to 15 weeks, or at least about 16 to 20 weeks after the initial dosing.

In some embodiments, the subsequent administration step can comprise administering a second SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 (e.g., that is different from a first SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 administered in an initial administration step) to the subject one or more subsequent times until a desired clinical outcome and/or measurement of interest is achieved in the subject.

In some embodiments, the subsequent administration step can comprise administering a second SHP2 inhibitor and/or a binding partner (e.g., that is different from a first SHP2 inhibitor and/or a binding partner administered in an initial administration step) to the subject one or more subsequent times until a desired clinical outcome and/or measurement of interest is achieved in the subject.

For example, the subsequent administration step(s) can be at least about 1 week, at least about 2 to 4 weeks, at least about 5 weeks, at least about 6 to 9 weeks, at least about 10 weeks, at least about 11 to 15 weeks, or at least about 16 to 20 weeks after the initial dosing.

In some embodiments, the subsequently administered SHP2 inhibitor and/or inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered via the same administration route as the originally administered SHP2 inhibitor and/or inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the subsequently administered SHP2 inhibitor and/or binding partner is administered via the same administration route as the originally administered SHP2 inhibitor and/or binding partner.

In some embodiments, the subsequently administered SHP2 inhibitor and/or inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 is administered via a different administration route as the originally administered SHP2 inhibitor and/or inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the subsequently administered SHP2 inhibitor and/or binding partner is administered via a different administration route as the originally administered SHP2 inhibitor and/or binding partner.

In certain embodiments, the SHP2 inhibitor is administered before the administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the SHP2 inhibitor is administered before the administration of the binding partner.

In certain embodiments, the SHP2 inhibitor is administered simultaneously with the administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the SHP2 inhibitor is administered simultaneously with the administration of the binding partner.

In certain embodiments, the SHP2 inhibitor is administered after the administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the SHP2 inhibitor is administered after the administration of the binding partner.

In some embodiments, the inhibitors of expression or function or degraders of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 are administered two or more times and the SHP2 inhibitor is administered before and/or between each of the administrations of the inhibitors of expression or function or degraders of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the binding partners are administered two or more times and the SHP2 inhibitor is administered before and/or between each of the administrations of the binding partners.

In some embodiments, the SHP2 inhibitor is administered simultaneously with the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 or not simultaneously (e.g., sequentially in any combination). For example, in a method comprising administering an SHP2 inhibitor and an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, they can be administered separately (e.g., the SHP2 inhibitor separately from the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1). Further, the SHP2 inhibitor can be administered prior to, subsequent to, prior to and subsequent to, or at the same time as the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the SHP2 inhibitor is administered simultaneously with the binding partner or not simultaneously (e.g., sequentially in any combination). For example, in a method comprising administering an SHP2 inhibitor and a binding partner, they can be administered separately (e.g., the SHP2 inhibitor separately from the binding partner). Further, the SHP2 inhibitor can be administered prior to, subsequent to, prior to and subsequent to, or at the same time as the binding partner.

In certain embodiments, the SHP2 inhibitor can be administered about 2 hours to about 72 hours, about 2 hours to about 48 hours, about 2 hours to about 24 hours, about 2 hours to about 12 hours, about 2 hours to about 6 hours, about 2 hours to about 4 hours, about 1 hour to about 72 hours, about 1 hour to about 48 hours, about 1 hour to about 24 hours, about 1 hour to about 12 hours, about 1 hour to about 6 hours, about 1 hour to about 4 hours, about 1 hour to about 2 hours, about 6 hours to about 48 hours, about 6 hours to about 24 hours, about 12 hours to about 48 hours, or about 24 hours to about 48 hours prior to and/or subsequent to administration of the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In certain embodiments, the SHP2 inhibitor can be administered about 2 hours to about 72 hours, about 2 hours to about 48 hours, about 2 hours to about 24 hours, about 2 hours to about 12 hours, about 2 hours to about 6 hours, about 2 hours to about 4 hours, about 1 hour to about 72 hours, about 1 hour to about 48 hours, about 1 hour to about 24 hours, about 1 hour to about 12 hours, about 1 hour to about 6 hours, about 1 hour to about 4 hours, about 1 hour to about 2 hours, about 6 hours to about 48 hours, about 6 hours to about 24 hours, about 12 hours to about 48 hours, or about 24 hours to about 48 hours prior to and/or subsequent to administration of the binding partner.

In some embodiments, the SHP2 inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to administering the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and/or subsequent to administering the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the SHP2 inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to administering the binding partner. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and/or subsequent to administering the binding partner.

In some embodiments, the SHP2 inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to and subsequent to administering the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and subsequent to administering the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the SHP2 inhibitor is administered at least about 2 hours, at least about 4 hours, at least about 6 hours, at least about 8 hours, at least about 12 hours, at least about 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks prior to and subsequent to administering the binding partner. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days prior to and subsequent to administering the binding partner.

In some embodiments, the SHP2 inhibitor is administered at least 2 hours, at least about 4 hours, at least 6 hours, at least about 8 hours, at least about 12 hours, at least 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks after administering inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days after administering the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1.

In some embodiments, the SHP2 inhibitor is administered at least 2 hours, at least about 4 hours, at least 6 hours, at least about 8 hours, at least about 12 hours, at least 16 hours, at least about 18 hours, at least about 1 day, at least about 1.5 days, at least about 2 days, at least about 3 days, at least about 4 days, at least about 5 days, at least about 6 days, at least about 1 week, or at least about 2 weeks after administering the binding partner. In another embodiment, the SHP2 inhibitor is administered about 2 hours to about 48 hours, about 4 hours to about 24 hours, about 4 hours to about 12 hours, about 8 hours to about 24 hours, about 12 hours to about 24 hours, about 1 day to about 14 days, about 1 day to about 7 days, about 1 day to about 6 days, about 1 day to about 5 days, about 1 day to about 3 days, about 1 day to about 2 days, about 2 days to about 7 days, about 4 days to about 7 days, or about 5 days to about 7 days after administering the binding partner.

Administration in vivo can be by any suitable route including, but not limited to, oral, parenteral, intrathecal, intra-arterial, intraperitoneal, intravenous, subcutaneous, topical, intracranial, intratumoral, intranasal, or intramuscular. Systemic modes of administration include, but are not limited to, parenteral and oral routes. Parenteral routes include, but are not limited to, subcutaneous, intraosseous, intravenous, intranasal, intradermal, intraarterial, intramuscular, and intraperitoneal routes. Some specific examples are intravenous infusion, nasal instillation, and intravitreal injection. Local modes of administration include, but are not limited to, intracerebroventricular, intrathecal, intraparenchymal (e.g., localized intraparenchymal delivery to the cerebral cortex, temporal cortex, striatum, tegmentum, precentral gyrus, hippocampus, thalamus, frontal cortex, hypothalamus, cerebellum, amygdala, medulla, tectum, or substantia nigra), intravitreal, subconjunctival, intraocular, subretinal, intraorbital, and transscleral routes. Significantly smaller amounts of the active components may elicit an effect(s) when administered locally (e.g., intravitreal or intraparenchymal) compared to when administered systemically (e.g., intravenously). Administration locally may also reduce or eliminate the prevalence of potentially toxic side effects that may occur when the component(s) are administered systemically.

Administration in vivo can be by any suitable route including, but not limited to, intravenous, intracranial, subcutaneous, parenteral, intratumoral, intraperitoneal, oral, intra-arterial, intranasal, intrathecal, topical, or intramuscular.

The number of dosages and the frequency of administration can depend several factors. The administration of an SHP2 inhibitor and/or an inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 into the cell or subject can be performed one or more times over a specific period of time. In some embodiments, the administration can be performed only once over a period of time, at least two to three times over a period of time, at least four to five times over a period of time, at least six to seven times over a period of time, at least eight to nine times over a period of time, at least ten times over a period of time, at least eleven to twelve times over a period of time, at least thirteen to fourteen times over a period of time, at least fifteen times over a period of time, at least sixteen to nineteen times over a period of time, at least twenty times over a period of time, or at least twenty-two to thirty times over a period of time. In some methods, a single administration of the SHP2 inhibitor and/or the inhibitor of expression or function or a degrader of one or more proteins selected from VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1, is sufficient to achieve the desired effect(s). In some embodiments, more than one administration may be beneficial to maximize desired therapeutic effect.

The number of dosages and the frequency of administration can depend on several factors. The administration of an SHP2 inhibitor and/or a binding partner into the cell or subject can be performed one or more times over a specific period of time. In some embodiments, the administration can be performed only once over a period of time, at least two to three times over a period of time, at least four to five times over a period of time, at least six to seven times over a period of time, at least eight to nine times over a period of time, at least ten times over a period of time, at least eleven to twelve times over a period of time, at least thirteen to fourteen times over a period of time, at least fifteen times over a period of time, at least sixteen to nineteen times over a period of time, at least twenty times over a period of time, or at least twenty-two to thirty times over a period of time. In some methods, a single administration of the SHP2 inhibitor and/or the binding partner, is sufficient to achieve the desired effect(s). In some embodiments, more than one administration may be beneficial to maximize desired therapeutic effect.

In some embodiments, the SHP2 inhibitor and/or the inhibitor of expression or function or degrader of said one or more proteins is administered orally or intravenously.

In some embodiments, the SHP2 inhibitor and/or binding partner is administered orally or intravenously.

In some embodiments, the SHP2 inhibitor and/or the inhibitor of expression or function or degrader of said one or more proteins may be carried out in combination with one or more of a known therapy including, but not limited to, surgery, radiation therapy, chemotherapy, photodynamic therapy, and/or immunotherapy.

In some embodiments, the SHP2 inhibitor and/or the binding partner may be carried out in combination with one or more of a known therapy including, but not limited to, surgery, radiation therapy, chemotherapy, photodynamic therapy, and/or immunotherapy.

In some embodiments, the SHP2 inhibitor is selected from BBP-398, TNO155, RMC-4630, JAB-3068, RLY-1971, and ERAS-601, pharmaceutically acceptable salts thereof, and any combinations thereof.

In some embodiments, the KRAS mutant cancer is resistant to a treatment with said SHP2 inhibitor when said SHP2 inhibitor is administered in the absence of said inhibitor of expression or function or degrader of said one or more proteins.

### Kits

### Kits Comprising KRAS Inhibitors

The present disclosure further provides a kit which may contain any of various compositions of the present disclosure, including any of various KRAS inhibitors, inhibitors of expression or function or degraders of one or more of various proteins (e.g., VRK1, RIOK2, PKN2, EXT1, EXT2, ELP1, ELP2, ELP3, ELP4, ELP5, ELP6, PGD, PGM2, ROCK1, and ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2 proteins) described herein.

In one aspect, a kit may comprise (i) a KRAS inhibitor, (ii) an inhibitor of expression or function or a degrader of one or more proteins selected from VRK1, RIOK2, PKN2, EXT1, EXT2, ELP1, ELP2, ELP3, ELP4, ELP5, ELP6, PGD, SHP2, SHOC2, PGM2, ROCK1, ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, and (iii) optionally, instructions for use.

In certain embodiments, a kit may comprise (i) a KRAS inhibitor, (ii) an inhibitor of expression or function or a degrader of one or more proteins selected from VRK1, RIOK2, PKN2, EXT1, EXT2, ELP2, ELP3, ELP5, PKN2, PGD, SHP2, SHOC2, PGM2, ROCK1, and ROCK2, AP2S1, ERBB3, GRB2, CRK, SRC, PAK2, NDST1, SHOC2, IPO11, YAP, WWTR1, TEAD, MTOR, RPTOR, TFIIIC, GTF3C1, TBP, HSD17B10, POP5, RPP21, RTCB, TSEN2, URM1, ADAT3, MOCS3, KTI12, IARS, YARS, SEPSECS, PARS2, YARS2, DARS2, and LARS2, and (iii) optionally, instructions for use.

In some embodiments, a kit can comprise: (a) a container that contains a pharmaceutical composition described herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, a kit may further comprise, one or more of (i) a diluent, (ii) a buffer, (iii) a filter (iv) a syringe, and/or (v) a needle.

In some embodiments, the components of the kit may be provided in one or more liquid solutions. A liquid solution described herein may be an aqueous solution such as a sterile aqueous solution. The components of the kit may also be provided as solids, which may be converted into liquids such as by addition of suitable solvents, which may be provided in another distinct container.

In some aspects, a pharmaceutical composition described herein may be lyophilized.

In some embodiments, kits may comprise a lyophilized formulation described herein in a suitable container and instructions for its reconstitution and/or use. Non-limiting examples of suitable containers include, e.g., syringes (such as dual chamber syringes), vials (such as dual chamber vials), bottles, and test tubes. In various embodiments, a container may be a multi-use container. The container may be formed from a variety of materials such as plastic or glass. The kit and/or container may contain instructions upon or accompanying the container which can denote directions for reconstitution of, e.g., a lyophilized formulation and/or use of the kit. In some embodiments, a label may denote that the lyophilized formulation is to be reconstituted to an appropriate concentration. The label may denote that the formulation is useful or intended for any route of administration disclosed herein.

The container containing the formulation may be a multi-use vial, which may allow for repeat administrations (e.g., from 2-6 administrations) of a reconstituted formulation. The kit may further comprise a second container comprising a suitable diluent (e.g., sodium bicarbonate solution).

Upon mixing of the diluent and a lyophilized formulation, a final concentration in the reconstituted formulation can reached. The kit may further include other materials desirable from a commercial and/or user perspective, including, e.g., other filters, needles, syringes, buffers, diluents, and/or package inserts which may comprise, e.g., instructions for use.

Kits may contain a single container that contains the formulation of the pharmaceutical composition with or without other components (e.g., other compounds or pharmaceutical compositions of such other compounds) or may have a separate container for each component.

Kits may include a formulation of the disclosure packaged for use in combination with the co-administration of a second compound (such as adjuvants (e.g., GM-CSF, a natural product, a hormone or antagonist, an anti-angiogenesis agent or inhibitor, an apoptosis-inducing agent or a chelator a chemotherapeutic agent) or a pharmaceutical composition thereof. The components of the kit may pre-mixed and/or pre-complexed or each component of the kit may be in a separate distinct container prior to administration to a patient.

In some embodiments, the container of a therapeutic kit may be a vial, flask, test tube, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiments, a kit may contain an apparatus (e.g., syringes, one or more needles, pipettes, eye droppers, etc.) which may permit administration of agents of the disclosure which are components of the kit.

### Kits Comprising SHP2 Inhibitors

The present disclosure further provides a kit which may contain any of various compositions of the present disclosure, including any of various SHP2 inhibitors, inhibitors of expression or function or degraders of one or more of various proteins (e.g., VRK1, RIOK2, ELP4, ELP5, ENO1, GAPDH, MARS2, ATP6V1F, PRMT5, COQ2, DBR1, DTYMK, DKC1, RNMT, PPP1R8, HSD17B10, DOLK, ALG1, UROD, POLR3H, PGD, TSEN2, RNASEH2A, GUK1, TSFM, NELFB, DOHH, EXOSC5, RPE, CSTF1, RTEL1, WARS2, UTP23, POLG2, THG1L, RARS2, RAD51D, LARS2, SDHB, CPSF4, PDPK1, DDX10, VARS2, PDSS2, PSMG4, DHX33, COASY, VHL, RNGTT, PPP1R2, NOL11, CTDNEP1, ISG20L2, ERCC2, TOP3A, MTG2, BRF1, PIK3C3, IARS, AURKAIP1, UQCRFS1, PRMT1, DDX59, MARS, TOE1, SARS2, CDIPT, YARS, CARS2, PPP2R4, RPP21, UGP2, DPAGT1, PYROXD1, MTOR, HARS2, NARS, TSC1, POLR3C, QRSL1, RPIA, SDHC, DDX56, EIF4E, DDX46, IMPDH2, SOD2, UBE2M, GATC, TSC2, PMPCA, TSEN54, FOXM1, FARS2, CTPS1, PARS2, ALG2, EIF2B3, CMPK1, DHDDS, SAE1, NARS2, PNKP, PDSS1, POLR3K, AHCY, NAE1, UBIAD1, RPUSD4, EARS2, GMPPB, LIAS, PPP4C, NSUN4, DLD, TRMT5, AASDHPPT, EIF5A, POT1, DHX9, LONP1, PPP6C, SKIV2L2, PTDSS1, USP5, VPS52, TKT, TRMT61A, N6AMT1, GGPS1, EFTUD1, ACAD9, SETD1A, IPO11, EIF3I, METTL16, MASTL, DDX51, ADAT3, ZNRD1, OGT, IDI1, IMP4, FTSJ3, EXOSC8, GSG2, PI4KA, NSMCE2, DDX52, DDOST, CSNK2B, UBA2, RABGGTA, SOD1, TRIT1, TYMS, RNF168, UBE2I, GARS, IPO13, SMARCB1, EIF2B1, RNASEH1, MCAT, XRN2, POP5, CS, FNTB, DARS2, TFRC, SLC7A6OS, GNB2L1, GFER, ATP6AP2, SLC25A19, and PEAR1 proteins) described herein.

In one aspect, a kit may comprise (i) an SHP2 inhibitor, (ii) an inhibitor of expression or function or a degrader of one or more proteins selected from VRK1, RIOK2, ELP4, and ELP5, and (iii) optionally, instructions for use.

In certain embodiments, a kit may comprise (i) an SHP2 inhibitor, (ii) a binding partner of one or more proteins selected from TFRC, SLC7A6OS, GNB2L1, GFER, ATP6AP2, SLC25A19, and PEAR1, and (iii) optionally, instructions for use.

In some embodiments, a kit can comprise: (a) a container that contains a pharmaceutical composition described herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, a kit may further comprise, one or more of (i) a diluent, (ii) a buffer, (iii) a filter (iv) a syringe, and/or (v) a needle.

In some embodiments, the components of the kit may be provided in one or more liquid solutions. A liquid solutions described herein may be an aqueous solution such as a sterile aqueous solution. The components of the kit may also be provided as solids, which may be converted into liquids such as by addition of suitable solvents, which may be provided in another distinct container.

In some aspects, a pharmaceutical composition described herein may be lyophilized.

In some embodiments, kits may comprise a lyophilized formulation described herein in a suitable container and instructions for its reconstitution and/or use. Non-limiting examples of suitable containers include, e.g., syringes (such as dual chamber syringes), vials (such as dual chamber vials), bottles, and test tubes. In various embodiments, a container may be a multi-use container. The container may be formed from a variety of materials such as plastic or glass. The kit and/or container may contain instructions upon or accompanying the container which can denote directions for reconstitution of, e.g., a lyophilized formulation and/or use of the kit. In some embodiments, a label may denote that the lyophilized formulation is to be reconstituted to an appropriate concentration. The label may denote that the formulation is useful or intended for any route of administration disclosed herein.

The container containing the formulation may be a multi-use vial, which may allow for repeat administrations (e.g., from 2-6 administrations) of a reconstituted formulation. The kit may further comprise a second container comprising a suitable diluent (e.g., sodium bicarbonate solution).

Upon mixing of the diluent and a lyophilized formulation, a final concentration in the reconstituted formulation can reached. The kit may further include other materials desirable from a commercial and/or user perspective, including, e.g., other filters, needles, syringes, buffers, diluents, and/or package inserts which may comprise, e.g., instructions for use.

Kits may contain a single container that contains the formulation of the pharmaceutical composition with or without other components (e.g., other compounds or pharmaceutical compositions of such other compounds) or may have a separate container for each component.

Kits may include a formulation of the disclosure packaged for use in combination with the co-administration of a second compound (such as adjuvants (e.g., GM-CSF, a natural product, a hormone or antagonist, an anti-angiogenesis agent or inhibitor, an apoptosis-inducing agent or a chelator a chemotherapeutic agent) or a pharmaceutical composition thereof. The components of the kit may pre-mixed and/or pre-complexed or each component of the kit may be in a separate distinct container prior to administration to a patient.

In some embodiments, the container of a therapeutic kit may be a vial, flask, test tube, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiments, a kit may contain an apparatus (e.g., syringes, one or more needles, pipettes, eye droppers, etc.) which may permit administration of agents of the disclosure which are components of the kit.

### EXAMPLES

The following examples are provided to further describe some of the embodiments disclosed herein. The examples are intended to illustrate, not to limit, the disclosed embodiments.

### Example 1. Genome-Wide CRISPR/Cas9 Screens Identify G12Ci Synthetic Lethal Genes

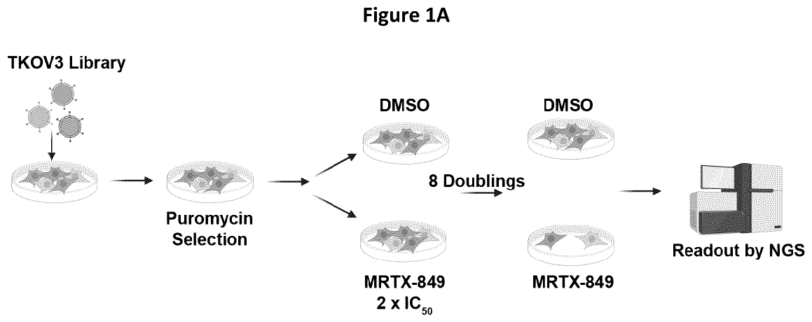

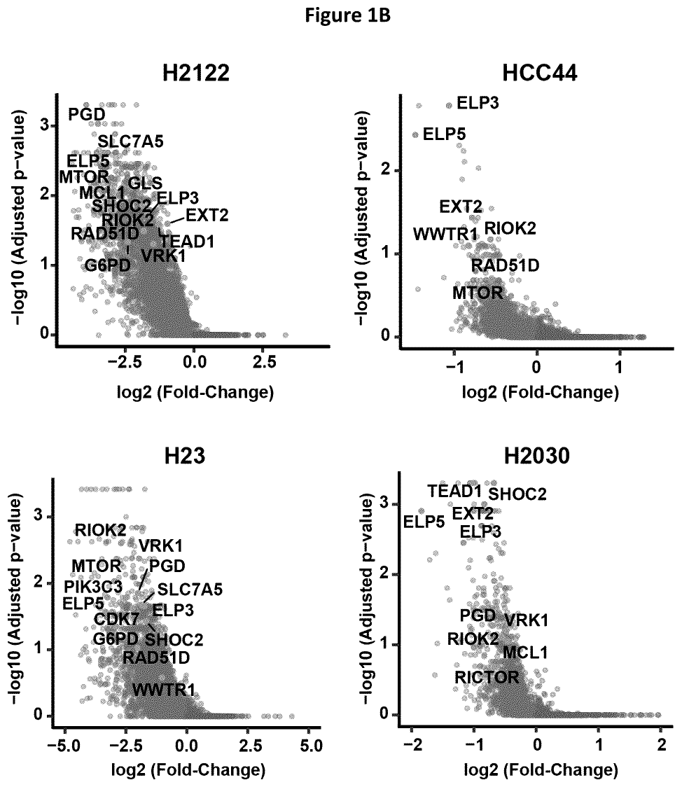

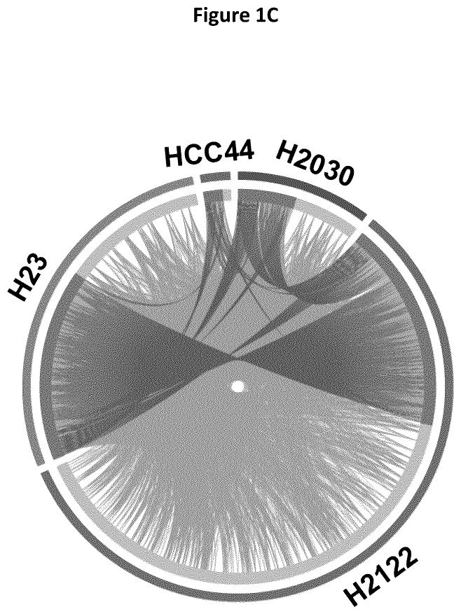

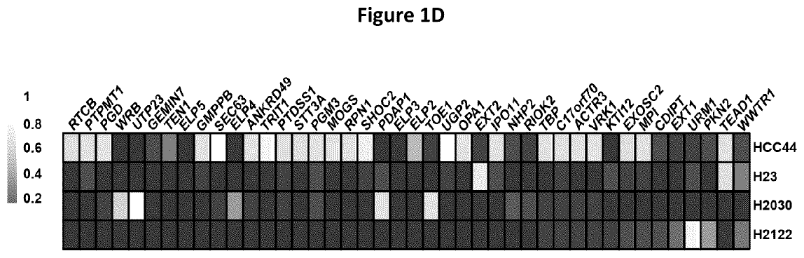

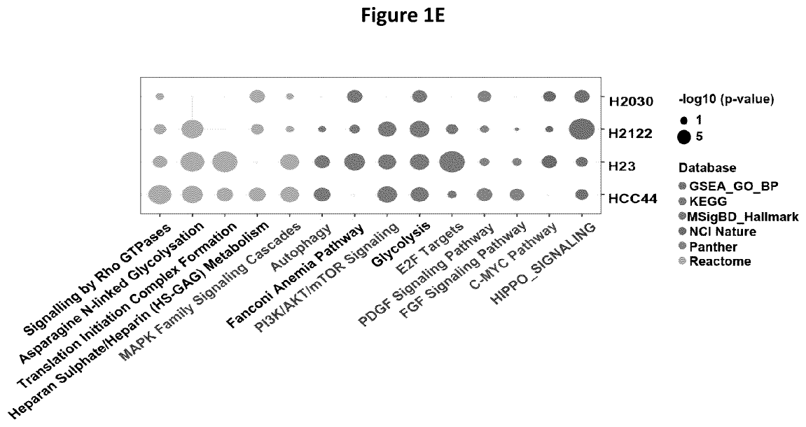

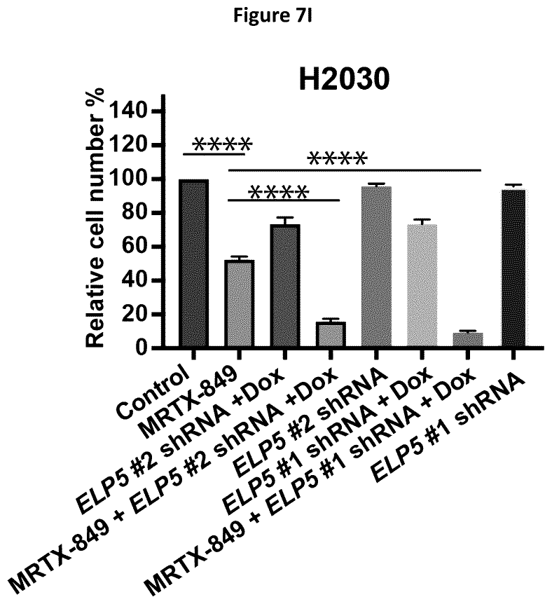

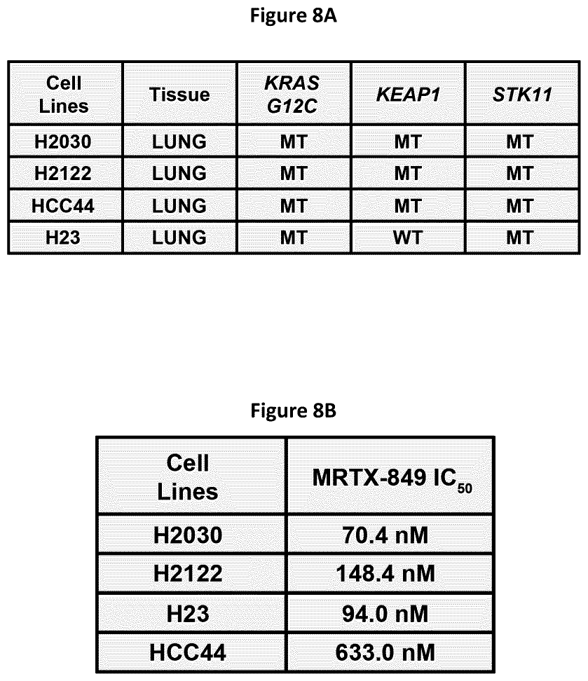

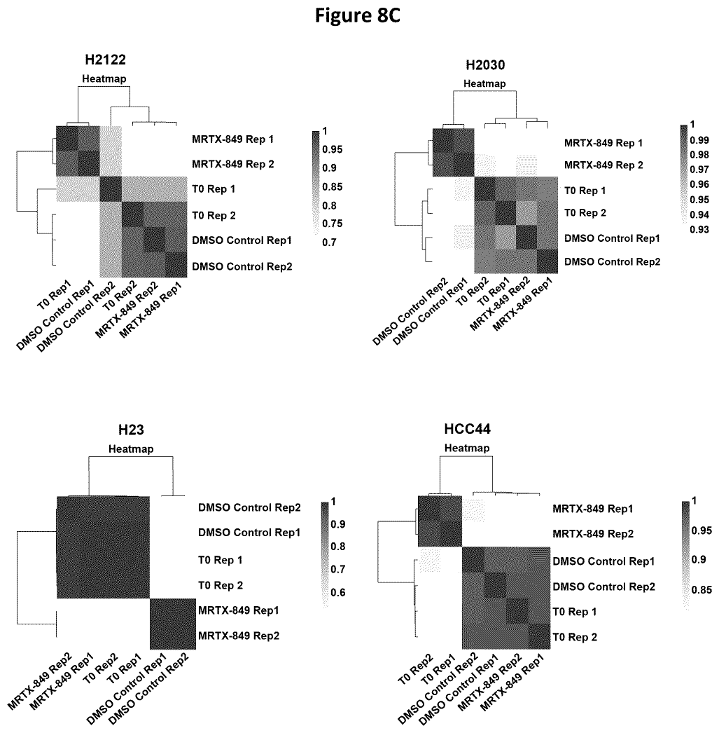

To identify genes that enhance G12Ci efficacy, pooled genome-wide CRISPR/Cas9 “dropout” screens were performed on four KRAS^{G12C}-mutant NSCLC lines co-mutant/deleted for STK11; three lines (excluding H23) also have KEAP1 mutations FIG. 8A. Notably, KRAS^{G12C}-mutant NSCLC with KEAP1 and possibly STK11 mutations are more resistant to single-agent G12Ci treatment, as well as conventional chemoradiation and immune therapy (6,16,17). Each cell line was transduced with the TKOV3 lentiviral CRISPR library, which targets 18,053 protein-coding genes with sgRNA RNAs per gene (18,19), at an M.O.I of 0.3 and 500× representation for each sgRNA. Infected cells were cultured for eight population doublings in vehicle (DMSO) or with adagrasib (MRTX-849) added at twice the IC₅₀ concentration (2×IC₅₀) for each line (FIG. 8B). Genomic DNA (gDNA) was sequenced, and gene “dropout” was assessed by robust rank aggregation as implemented in MaGeCK (20). Log₂ fold-changes were calculated and displayed in volcano plots (FIG. 1B; Table 1). Biological replicates were strongly correlated, indicating that the screens were high-quality (FIG. 8C).

TABLE 1
Synthetic lethal (i.e., dropout) genes (FDR <0.1) from MRTX-849 (adagrasib) CRISPR/Cas9 screens of four NSCLC cell lines (MaGeCK analysis)

| | | | | | | | | | | | | | |
|---|---|---|---|---|---|---|---|---|---|---|---|---|---|
| id | num | neg\|score | neg\|p-value | neg\|fdr | neg\|rank | neg\|goodsgrna | neg\|lfc | pos\|score | pos\|p-value | pos\|fdr | pos\|rank | pos\|goodsgrna | pos\|lfc |

H2122 MRTX SL Gene FDR 0.1

| | | | | | | | | | | | | | |
|---|---|---|---|---|---|---|---|---|---|---|---|---|---|
| RTCB | 4 | 5.92E−12 | 2.74E−07 | 0.000495 | 1 | 4 | −4.6534 | 1 | 1 | 1 | 18053 | 0 | −4.6534 |
| RNASEH2A | 4 | 1.01E−08 | 2.74E−07 | 0.000495 | 2 | 4 | −3.1058 | 1 | 1 | 1 | 18047 | 0 | −3.1058 |
| HIRA | 4 | 2.62E−08 | 2.74E−07 | 0.000495 | 3 | 4 | −2.5823 | 1 | 1 | 1 | 18052 | 0 | −2.5823 |
| COA6 | 4 | 3.28E−08 | 2.74E−07 | 0.000495 | 4 | 4 | −4.7243 | 1 | 1 | 1 | 18051 | 0 | −4.7243 |
| TCOF1 | 4 | 1.03E−07 | 2.74E−07 | 0.000495 | 5 | 4 | −2.991 | 1 | 1 | 1 | 18050 | 0 | −2.991 |
| PHB | 4 | 1.04E−07 | 2.74E−07 | 0.000495 | 6 | 4 | −2.094 | 1 | 1 | 1 | 18049 | 0 | −2.094 |
| LSM10 | 4 | 1.17E−07 | 2.74E−07 | 0.000495 | 7 | 4 | −2.8989 | 1 | 1 | 1 | 18048 | 0 | −2.8989 |
| JMJD6 | 4 | 1.61E−07 | 2.74E−07 | 0.000495 | 8 | 4 | −3.9253 | 1 | 1 | 1 | 18046 | 0 | −3.9253 |
| PET117 | 4 | 1.64E−07 | 2.74E−07 | 0.000495 | 9 | 4 | −3.9178 | 1 | 1 | 1 | 18045 | 0 | −3.9178 |
| CPOX | 4 | 1.71E−07 | 2.74E−07 | 0.000495 | 10 | 4 | −3.3557 | 1 | 1 | 1 | 18044 | 0 | −3.3557 |
| PTPMT1 | 4 | 1.83E−07 | 8.23E−07 | 0.000928 | 11 | 4 | −2.6365 | 1 | 1 | 1 | 18043 | 0 | −2.6365 |
| RCL1 | 4 | 1.88E−07 | 8.23E−07 | 0.000928 | 12 | 4 | −3.2434 | 1 | 1 | 1 | 18042 | 0 | −3.2434 |
| MECR | 4 | 2.08E−07 | 8.23E−07 | 0.000928 | 13 | 4 | −2.9305 | 1 | 1 | 1 | 18041 | 0 | −2.9305 |
| PGD | 4 | 2.22E−07 | 8.23E−07 | 0.000928 | 14 | 4 | −3.4918 | 1 | 1 | 1 | 18040 | 0 | −3.4918 |
| ALG1 | 4 | 2.29E−07 | 8.23E−07 | 0.000928 | 15 | 4 | −2.8343 | 1 | 1 | 1 | 18039 | 0 | −2.8343 |
| DOLK | 4 | 2.58E−07 | 8.23E−07 | 0.000928 | 16 | 4 | −3.7415 | 1 | 1 | 1 | 18029 | 0 | −3.7415 |
| MRPL53 | 4 | 4.40E−07 | 1.37E−06 | 0.001303 | 17 | 4 | −3.6342 | 1 | 1 | 1 | 18038 | 0 | −3.6342 |
| WRB | 4 | 4.60E−07 | 1.37E−06 | 0.001303 | 18 | 4 | −2.8688 | 1 | 1 | 1 | 18037 | 0 | −2.8688 |
| C7orf55-LUC7L2 | 4 | 4.92E−07 | 1.37E−06 | 0.001303 | 19 | 4 | −2.8727 | 1 | 1 | 1 | 18036 | 0 | −2.8727 |
| UTP23 | 4 | 5.95E−07 | 1.92E−06 | 0.00165 | 20 | 4 | −5.2085 | 1 | 1 | 1 | 18035 | 0 | −5.2085 |
| COQ2 | 4 | 6.12E−07 | 1.92E−06 | 0.00165 | 21 | 4 | −2.9734 | 0.99824 | 0.99827 | 1 | 17246 | 0 | −2.9734 |
| KDM2A | 4 | 8.00E−07 | 2.47E−06 | 0.001937 | 22 | 4 | −3.0224 | 1 | 1 | 1 | 18034 | 0 | −3.0224 |
| ATP6V1F | 4 | 8.21E−07 | 2.47E−06 | 0.001937 | 23 | 4 | −2.4757 | 1 | 1 | 1 | 18033 | 0 | −2.4757 |
| KBTBD2 | 4 | 9.44E−07 | 3.02E−06 | 0.002178 | 24 | 4 | −2.4184 | 1 | 1 | 1 | 18032 | 0 | −2.4184 |
| ENO1 | 4 | 1.00E−06 | 3.02E−06 | 0.002178 | 25 | 4 | −3.4532 | 0.99995 | 0.99995 | 1 | 17858 | 0 | −3.4532 |
| GPN3 | 4 | 1.07E−06 | 3.56E−06 | 0.002384 | 26 | 4 | −3.5503 | 1 | 1 | 1 | 18031 | 0 | −3.5503 |
| RFC5 | 4 | 1.11E−06 | 3.56E−06 | 0.002384 | 27 | 4 | −2.8244 | 1 | 1 | 1 | 18030 | 0 | −2.8244 |
| SLC7A5 | 4 | 1.46E−06 | 4.66E−06 | 0.002418 | 28 | 4 | −3.2912 | 1 | 1 | 1 | 18028 | 0 | −3.2912 |
| SLC33A1 | 4 | 1.57E−06 | 4.66E−06 | 0.002418 | 29 | 4 | −3.1337 | 1 | 1 | 1 | 18027 | 0 | −3.1337 |
| POLR3H | 4 | 1.58E−06 | 4.66E−06 | 0.002418 | 30 | 4 | −3.2707 | 1 | 1 | 1 | 18026 | 0 | −3.2707 |
| DKC1 | 4 | 1.67E−06 | 4.66E−06 | 0.002418 | 31 | 4 | −4.2717 | 1 | 1 | 1 | 18025 | 0 | −4.2717 |
| GEMIN7 | 4 | 1.72E−06 | 5.21E−06 | 0.002418 | 32 | 4 | −3.1294 | 1 | 1 | 1 | 18024 | 0 | −3.1294 |
| NAE1 | 4 | 1.78E−06 | 5.21E−06 | 0.002418 | 33 | 4 | −2.5166 | 1 | 1 | 1 | 18023 | 0 | −2.5166 |
| RCC1 | 4 | 1.94E−06 | 5.21E−06 | 0.002418 | 34 | 4 | −2.0043 | 1 | 1 | 1 | 18022 | 0 | −2.0043 |
| RPE | 4 | 1.96E−06 | 5.21E−06 | 0.002418 | 35 | 4 | −4.0124 | 0.99999 | 0.99999 | 1 | 17951 | 0 | −4.0124 |
| GNB1L | 3 | 2.05E−06 | 7.40E−06 | 0.002844 | 36 | 3 | −3.5205 | 1 | 1 | 1 | 18021 | 0 | −3.5205 |
| TEN1 | 4 | 2.06E−06 | 5.21E−06 | 0.002418 | 37 | 4 | −4.3632 | 0.99997 | 0.99997 | 1 | 17904 | 0 | −4.3632 |
| SDHB | 4 | 2.06E−06 | 5.21E−06 | 0.002418 | 38 | 4 | −3.2516 | 1 | 1 | 1 | 18020 | 0 | −3.2516 |
| ZNF236 | 4 | 2.07E−06 | 5.21E−06 | 0.002418 | 39 | 3 | −2.853 | 0.71485 | 0.78601 | 1 | 12921 | 1 | −2.853 |
| MTG2 | 4 | 2.20E−06 | 5.76E−06 | 0.002418 | 40 | 4 | −2.8687 | 1 | 1 | 1 | 18019 | 0 | −2.8687 |
| QRSL1 | 4 | 2.30E−06 | 5.76E−06 | 0.002418 | 41 | 4 | −3.8468 | 1 | 1 | 1 | 18018 | 0 | −3.8468 |
| SCO2 | 4 | 2.34E−06 | 5.76E−06 | 0.002418 | 42 | 4 | −2.1304 | 1 | 1 | 1 | 18017 | 0 | −2.1304 |
| VARS2 | 4 | 2.37E−06 | 5.76E−06 | 0.002418 | 43 | 4 | −3.5442 | 0.99996 | 0.99996 | 1 | 17872 | 0 | −3.5442 |
| BTAF1 | 4 | 2.40E−06 | 5.76E−06 | 0.002418 | 44 | 4 | −2.5146 | 1 | 1 | 1 | 18016 | 0 | −2.5146 |
| TRAPPC3 | 4 | 2.49E−06 | 6.31E−06 | 0.002588 | 45 | 4 | −2.9804 | 1 | 1 | 1 | 18015 | 0 | −2.9804 |
| ELP5 | 4 | 2.54E−06 | 6.86E−06 | 0.00269 | 46 | 4 | −3.4622 | 1 | 1 | 1 | 18014 | 0 | −3.4622 |
| HNRNPD | 4 | 2.62E−06 | 6.86E−06 | 0.00269 | 47 | 4 | −2.7031 | 1 | 1 | 1 | 18013 | 0 | −2.7031 |
| MAD2L2 | 4 | 2.69E−06 | 7.95E−06 | 0.002991 | 48 | 4 | −2.8392 | 1 | 1 | 1 | 18012 | 0 | −2.8392 |
| PTCD3 | 4 | 2.88E−06 | 1.01E−05 | 0.003481 | 49 | 4 | −2.0636 | 1 | 1 | 1 | 18011 | 0 | −2.0636 |
| SARS2 | 4 | 3.03E−06 | 1.01E−05 | 0.003481 | 50 | 4 | −2.876 | 1 | 1 | 1 | 18010 | 0 | −2.876 |
| CSTF1 | 4 | 3.08E−06 | 1.07E−05 | 0.003481 | 51 | 4 | −2.6813 | 1 | 1 | 1 | 18009 | 0 | −2.6813 |
| LIN52 | 4 | 3.34E−06 | 1.12E−05 | 0.003481 | 52 | 4 | −2.1287 | 1 | 1 | 1 | 18008 | 0 | −2.1287 |
| MRPL13 | 4 | 3.35E−06 | 1.12E−05 | 0.003481 | 53 | 4 | −2.7409 | 1 | 1 | 1 | 18007 | 0 | −2.7409 |
| DOHH | 4 | 3.47E−06 | 1.12E−05 | 0.003481 | 54 | 4 | −2.0355 | 1 | 1 | 1 | 18006 | 0 | −2.0355 |
| GTF2H1 | 4 | 3.52E−06 | 1.12E−05 | 0.003481 | 55 | 4 | −1.6704 | 1 | 1 | 1 | 18005 | 0 | −1.6704 |
| MRPL4 | 4 | 3.53E−06 | 1.12E−05 | 0.003481 | 56 | 4 | −2.0256 | 1 | 1 | 1 | 18004 | 0 | −2.0256 |
| DDX59 | 3 | 3.69E−06 | 1.18E−05 | 0.003481 | 57 | 3 | −3.4244 | 1 | 1 | 1 | 18003 | 0 | −3.4244 |
| DBR1 | 4 | 3.72E−06 | 1.18E−05 | 0.003481 | 58 | 4 | −4.4477 | 1 | 1 | 1 | 18002 | 0 | −4.4477 |
| SIRT6 | 4 | 3.74E−06 | 1.18E−05 | 0.003481 | 59 | 4 | −1.9362 | 1 | 1 | 1 | 18001 | 0 | −1.9362 |
| RABGGTB | 4 | 3.79E−06 | 1.18E−05 | 0.003481 | 60 | 4 | −5.4361 | 1 | 1 | 1 | 18000 | 0 | −5.4361 |
| PCYT2 | 4 | 3.95E−06 | 1.23E−05 | 0.003481 | 61 | 4 | −3.1907 | 1 | 1 | 1 | 17999 | 0 | −3.1907 |
| WARS2 | 4 | 4.00E−06 | 1.23E−05 | 0.003481 | 62 | 4 | −2.5018 | 0.99974 | 0.99975 | 1 | 17648 | 0 | −2.5018 |
| N6AMT1 | 4 | 4.05E−06 | 1.23E−05 | 0.003481 | 63 | 4 | −3.1093 | 0.99994 | 0.99995 | 1 | 17847 | 0 | −3.1093 |
| TKT | 4 | 4.09E−06 | 1.23E−05 | 0.003481 | 64 | 4 | −3.5473 | 1 | 1 | 1 | 17998 | 0 | −3.5473 |
| ANAPC11 | 4 | 4.10E−06 | 1.26E−05 | 0.003503 | 65 | 4 | −2.2947 | 1 | 1 | 1 | 17997 | 0 | −2.2947 |
| NSMCE1 | 4 | 4.20E−06 | 1.29E−05 | 0.003525 | 66 | 4 | −2.8259 | 1 | 1 | 1 | 17996 | 0 | −2.8259 |
| SLC2A1 | 4 | 4.38E−06 | 1.40E−05 | 0.003768 | 67 | 4 | −2.4568 | 1 | 1 | 1 | 17995 | 0 | −2.4568 |
| DDB1 | 4 | 4.45E−06 | 1.45E−05 | 0.00385 | 68 | 4 | −3.0849 | 1 | 1 | 1 | 17994 | 0 | −3.0849 |
| MTMR9 | 4 | 4.61E−06 | 1.56E−05 | 0.00385 | 69 | 4 | −1.7604 | 1 | 1 | 1 | 17993 | 0 | −1.7604 |
| PARN | 4 | 4.66E−06 | 1.62E−05 | 0.00385 | 70 | 4 | −2.1396 | 1 | 1 | 1 | 17992 | 0 | −2.1396 |
| TNPO1 | 4 | 4.71E−06 | 1.67E−05 | 0.00385 | 71 | 4 | −3.2948 | 1 | 1 | 1 | 17991 | 0 | −3.2948 |
| MRPL15 | 4 | 4.72E−06 | 1.67E−05 | 0.00385 | 72 | 4 | −3.2364 | 1 | 1 | 1 | 17990 | 0 | −3.2364 |
| TIMMDC1 | 4 | 4.74E−06 | 1.67E−05 | 0.00385 | 73 | 4 | −1.6895 | 1 | 1 | 1 | 17989 | 0 | −1.6895 |
| OTUD5 | 4 | 4.76E−06 | 1.67E−05 | 0.00385 | 74 | 4 | −3.3865 | 1 | 1 | 1 | 17988 | 0 | −3.3865 |
| HSCB | 4 | 4.77E−06 | 1.67E−05 | 0.00385 | 75 | 4 | −3.174 | 0.99999 | 0.99999 | 1 | 17979 | 0 | −3.174 |
| URB1 | 4 | 4.91E−06 | 1.67E−05 | 0.00385 | 76 | 4 | −2.3412 | 0.99864 | 0.99865 | 1 | 17310 | 0 | −2.3412 |
| ARL2 | 4 | 4.98E−06 | 1.67E−05 | 0.00385 | 77 | 4 | −2.143 | 1 | 1 | 1 | 17987 | 0 | −2.143 |
| FDXR | 4 | 5.06E−06 | 1.73E−05 | 0.00385 | 78 | 4 | −2.5207 | 0.99526 | 0.99535 | 1 | 16988 | 0 | −2.5207 |
| DNAJC9 | 4 | 5.08E−06 | 1.73E−05 | 0.00385 | 79 | 4 | −2.4959 | 0.99999 | 1 | 1 | 17986 | 0 | −2.4959 |
| ABCB7 | 4 | 5.12E−06 | 1.73E−05 | 0.00385 | 80 | 4 | −2.4634 | 0.99999 | 1 | 1 | 17985 | 0 | −2.4634 |
| AHCY | 4 | 5.14E−06 | 1.73E−05 | 0.00385 | 81 | 4 | −3.3861 | 0.99987 | 0.99987 | 1 | 17744 | 0 | −3.3861 |
| DMAP1 | 4 | 5.51E−06 | 1.78E−05 | 0.003924 | 82 | 4 | −3.1738 | 0.99685 | 0.99689 | 1 | 17101 | 0 | −3.1738 |
| NDUFA1 | 4 | 5.69E−06 | 1.84E−05 | 0.003971 | 83 | 4 | −2.2485 | 0.99999 | 1 | 1 | 17984 | 0 | −2.2485 |
| CIT | 4 | 6.01E−06 | 1.95E−05 | 0.003971 | 84 | 4 | −2.601 | 0.99999 | 1 | 1 | 17983 | 0 | −2.601 |
| LIPT1 | 4 | 6.10E−06 | 1.95E−05 | 0.003971 | 85 | 4 | −3.2076 | 0.99999 | 1 | 1 | 17982 | 0 | −3.2076 |
| UBE2M | 4 | 6.14E−06 | 2.00E−05 | 0.003971 | 86 | 4 | −2.3201 | 0.99999 | 1 | 1 | 17981 | 0 | −2.3201 |
| ERCC2 | 4 | 6.18E−06 | 2.00E−05 | 0.003971 | 87 | 4 | −2.4543 | 0.99999 | 1 | 1 | 17980 | 0 | −2.4543 |
| SAE1 | 4 | 6.37E−06 | 2.00E−05 | 0.003971 | 88 | 4 | −2.1096 | 0.99999 | 0.99999 | 1 | 17978 | 0 | −2.1096 |
| MRPS6 | 4 | 6.39E−06 | 2.00E−05 | 0.003971 | 89 | 3 | −2.2944 | 0.60258 | 0.74192 | 1 | 12225 | 1 | −2.2944 |
| EDC3 | 4 | 6.44E−06 | 2.00E−05 | 0.003971 | 90 | 4 | −1.4402 | 0.99999 | 0.99999 | 1 | 17977 | 0 | −1.4402 |
| TOMM70A | 4 | 6.48E−06 | 2.00E−05 | 0.003971 | 91 | 4 | −3.2911 | 0.99433 | 0.99444 | 1 | 16928 | 0 | −3.2911 |
| TSC1 | 4 | 6.60E−06 | 2.06E−05 | 0.003992 | 92 | 4 | −1.7576 | 0.99999 | 0.99999 | 1 | 17976 | 0 | −1.7576 |
| MRPL39 | 4 | 6.71E−06 | 2.06E−05 | 0.003992 | 93 | 4 | −3.3445 | 0.98312 | 0.9832 | 1 | 16449 | 0 | −3.3445 |
| PHF12 | 4 | 7.03E−06 | 2.17E−05 | 0.004161 | 94 | 4 | −1.7763 | 0.99999 | 0.99999 | 1 | 17975 | 0 | −1.7763 |
| ACAD9 | 4 | 7.19E−06 | 2.22E−05 | 0.004177 | 95 | 4 | −1.9834 | 0.99999 | 0.99999 | 1 | 17974 | 0 | −1.9834 |
| PPP1CB | 4 | 7.23E−06 | 2.22E−05 | 0.004177 | 96 | 4 | −2.6325 | 0.97924 | 0.97932 | 1 | 16331 | 0 | −2.6325 |
| GMPPB | 3 | 7.29E−06 | 2.71E−05 | 0.004852 | 97 | 3 | −3.3017 | 0.99999 | 0.99999 | 1 | 17973 | 0 | −3.3017 |
| HGS | 4 | 7.72E−06 | 2.39E−05 | 0.00444 | 98 | 4 | −2.8466 | 0.99999 | 0.99999 | 1 | 17972 | 0 | −2.8466 |
| FUBP3 | 4 | 7.89E−06 | 2.55E−05 | 0.004698 | 99 | 4 | −2.1163 | 0.99269 | 0.9928 | 1 | 16830 | 0 | −2.1163 |
| LYRM4 | 4 | 8.17E−06 | 2.71E−05 | 0.004852 | 100 | 4 | −2.4215 | 0.99999 | 0.99999 | 1 | 17971 | 0 | −2.4215 |
| PPP1R2 | 4 | 8.20E−06 | 2.71E−05 | 0.004852 | 101 | 4 | −2.6328 | 0.99999 | 0.99999 | 1 | 17970 | 0 | −2.6328 |
| FDX1L | 4 | 8.44E−06 | 2.77E−05 | 0.004902 | 102 | 4 | −2.0714 | 0.99993 | 0.99994 | 1 | 17830 | 0 | −2.0714 |
| NAA25 | 4 | 8.52E−06 | 2.82E−05 | 0.004903 | 103 | 4 | −2.7584 | 0.99999 | 0.99999 | 1 | 17969 | 0 | −2.7584 |
| ASF1A | 4 | 8.56E−06 | 2.82E−05 | 0.004903 | 104 | 4 | −1.7945 | 0.99999 | 0.99999 | 1 | 17968 | 0 | −1.7945 |
| FARS2 | 4 | 8.75E−06 | 2.93E−05 | 0.004997 | 105 | 4 | −2.4367 | 0.99999 | 0.99999 | 1 | 17967 | 0 | −2.4367 |
| ALDOA | 4 | 8.77E−06 | 2.93E−05 | 0.004997 | 106 | 4 | −4.0134 | 0.99999 | 0.99999 | 1 | 17966 | 0 | −4.0134 |
| MARS2 | 4 | 8.94E−06 | 3.04E−05 | 0.005136 | 107 | 4 | −3.2943 | 0.99999 | 0.99999 | 1 | 17965 | 0 | −3.2943 |
| HSD17B10 | 4 | 9.35E−06 | 3.32E−05 | 0.005496 | 108 | 4 | −3.1955 | 0.99999 | 0.99999 | 1 | 17964 | 0 | −3.1955 |
| RNMT | 4 | 9.39E−06 | 3.32E−05 | 0.005496 | 109 | 4 | −1.9987 | 0.99999 | 0.99999 | 1 | 17963 | 0 | −1.9987 |
| TSSC1 | 4 | 9.54E−06 | 3.43E−05 | 0.005505 | 110 | 4 | −1.624 | 0.99999 | 0.99999 | 1 | 17962 | 0 | −1.624 |
| PPP6C | 4 | 9.77E−06 | 3.54E−05 | 0.005505 | 111 | 4 | −3.0023 | 0.99976 | 0.99977 | 1 | 17659 | 0 | −3.0023 |
| RTF1 | 4 | 9.80E−06 | 3.54E−05 | 0.005505 | 112 | 4 | −2.4246 | 0.99999 | 0.99999 | 1 | 17961 | 0 | −2.4246 |
| EAF1 | 4 | 1.00E−05 | 3.54E−05 | 0.005505 | 113 | 4 | −3.0132 | 0.99999 | 0.99999 | 1 | 17960 | 0 | −3.0132 |
| UROD | 4 | 1.01E−05 | 3.54E−05 | 0.005505 | 114 | 3 | −4.2563 | 0.62243 | 0.75024 | 1 | 12365 | 1 | −4.2563 |
| MTOR | 4 | 1.02E−05 | 3.54E−05 | 0.005505 | 115 | 4 | −3.5917 | 0.99999 | 0.99999 | 1 | 17959 | 0 | −3.5917 |
| DPAGT1 | 4 | 1.02E−05 | 3.54E−05 | 0.005505 | 116 | 4 | −3.0011 | 0.99978 | 0.99979 | 1 | 17677 | 0 | −3.0011 |
| SEC63 | 4 | 1.09E−05 | 3.59E−05 | 0.005543 | 117 | 4 | −2.4091 | 0.99974 | 0.99974 | 1 | 17647 | 0 | −2.4091 |
| GAPDH | 4 | 1.11E−05 | 3.70E−05 | 0.005664 | 118 | 4 | −2.0836 | 0.99999 | 0.99999 | 1 | 17958 | 0 | −2.0836 |
| GNB2L1 | 4 | 1.12E−05 | 3.81E−05 | 0.005783 | 119 | 4 | −2.8645 | 0.99999 | 0.99999 | 1 | 17957 | 0 | −2.8645 |
| DHX33 | 4 | 1.13E−05 | 3.87E−05 | 0.005803 | 120 | 4 | −2.0985 | 0.99999 | 0.99999 | 1 | 17956 | 0 | −2.0985 |
| CAB39 | 4 | 1.15E−05 | 3.92E−05 | 0.005803 | 121 | 4 | −2.9038 | 0.99999 | 0.99999 | 1 | 17955 | 0 | −2.9038 |
| ELP4 | 4 | 1.15E−05 | 3.92E−05 | 0.005803 | 122 | 4 | −2.7641 | 0.99999 | 0.99999 | 1 | 17954 | 0 | −2.7641 |
| PFAS | 4 | 1.17E−05 | 4.14E−05 | 0.006028 | 123 | 4 | −3.2561 | 0.99611 | 0.9962 | 1 | 17048 | 0 | −3.2561 |
| ANKRD49 | 4 | 1.18E−05 | 4.14E−05 | 0.006028 | 124 | 4 | −2.4467 | 0.99997 | 0.99997 | 1 | 17890 | 0 | −2.4467 |
| IRS2 | 4 | 1.21E−05 | 4.36E−05 | 0.006247 | 125 | 4 | −3.0594 | 0.99999 | 0.99999 | 1 | 17953 | 0 | −3.0594 |
| ERBB2 | 4 | 1.21E−05 | 4.36E−05 | 0.006247 | 126 | 4 | −2.5171 | 0.99999 | 0.99999 | 1 | 17952 | 0 | −2.5171 |
| ALG2 | 4 | 1.31E−05 | 4.80E−05 | 0.006564 | 127 | 4 | −3.0943 | 0.99959 | 0.99961 | 1 | 17560 | 0 | −3.0943 |
| GPS1 | 4 | 1.31E−05 | 4.80E−05 | 0.006564 | 128 | 4 | −2.8789 | 0.99999 | 0.99999 | 1 | 17950 | 0 | −2.8789 |
| APEX2 | 4 | 1.33E−05 | 4.85E−05 | 0.006564 | 129 | 4 | −2.461 | 0.99983 | 0.99984 | 1 | 17716 | 0 | −2.461 |
| MTX1 | 4 | 1.33E−05 | 4.85E−05 | 0.006564 | 130 | 4 | −1.9431 | 0.99999 | 0.99999 | 1 | 17949 | 0 | −1.9431 |
| MRPS14 | 4 | 1.34E−05 | 4.85E−05 | 0.006564 | 131 | 3 | −3.5554 | 0.66112 | 0.76424 | 1 | 12575 | 1 | −3.5554 |
| RPS16 | 4 | 1.35E−05 | 4.91E−05 | 0.006564 | 132 | 4 | −1.8657 | 0.99999 | 0.99999 | 1 | 17948 | 0 | −1.8657 |
| RFC3 | 4 | 1.35E−05 | 4.91E−05 | 0.006564 | 133 | 4 | −3.7679 | 0.99999 | 0.99999 | 1 | 17947 | 0 | −3.7679 |
| HUWE1 | 4 | 1.36E−05 | 4.91E−05 | 0.006564 | 134 | 4 | −2.4222 | 0.99999 | 0.99999 | 1 | 17946 | 0 | −2.4222 |
| NOC4L | 4 | 1.36E−05 | 4.91E−05 | 0.006564 | 135 | 4 | −1.6103 | 0.99999 | 0.99999 | 1 | 17945 | 0 | −1.6103 |
| UMPS | 4 | 1.38E−05 | 5.02E−05 | 0.006612 | 136 | 4 | −3.6347 | 0.99967 | 0.99969 | 1 | 17607 | 0 | −3.6347 |
| EMC1 | 4 | 1.41E−05 | 5.07E−05 | 0.006612 | 137 | 4 | −1.6023 | 0.99999 | 0.99999 | 1 | 17944 | 0 | −1.6023 |
| VMA21 | 4 | 1.44E−05 | 5.07E−05 | 0.006612 | 138 | 4 | −1.5459 | 0.99999 | 0.99999 | 1 | 17943 | 0 | −1.5459 |
| NDUFS1 | 4 | 1.44E−05 | 5.13E−05 | 0.006612 | 139 | 4 | −3.4742 | 0.99999 | 0.99999 | 1 | 17942 | 0 | −3.4742 |
| DCPS | 4 | 1.44E−05 | 5.13E−05 | 0.006612 | 140 | 3 | −2.5473 | 0.13002 | 0.27317 | 0.943647 | 5164 | 1 | −2.5473 |
| DHX15 | 4 | 1.46E−05 | 5.24E−05 | 0.006706 | 141 | 4 | −3.2925 | 0.99999 | 0.99998 | 1 | 17941 | 0 | −3.2925 |
| ATP6AP2 | 4 | 1.48E−05 | 5.29E−05 | 0.006728 | 142 | 4 | −3.6262 | 0.99999 | 0.99998 | 1 | 17940 | 0 | −3.6262 |
| TADA1 | 4 | 1.50E−05 | 5.40E−05 | 0.006794 | 143 | 4 | −1.8342 | 0.99998 | 0.99998 | 1 | 17939 | 0 | −1.8342 |
| PARS2 | 4 | 1.55E−05 | 5.46E−05 | 0.006794 | 144 | 4 | −3.1318 | 0.99998 | 0.99998 | 1 | 17938 | 0 | −3.1318 |
| HNF1B | 4 | 1.56E−05 | 5.46E−05 | 0.006794 | 145 | 4 | −2.2859 | 0.99998 | 0.99998 | 1 | 17937 | 0 | −2.2859 |
| CUL2 | 4 | 1.60E−05 | 5.90E−05 | 0.007241 | 146 | 4 | −2.4019 | 0.99998 | 0.99998 | 1 | 17936 | 0 | −2.4019 |
| CRTC3 | 4 | 1.60E−05 | 5.90E−05 | 0.007241 | 147 | 4 | −2.8888 | 0.99998 | 0.99998 | 1 | 17935 | 0 | −2.8888 |
| TSFM | 4 | 1.69E−05 | 6.33E−05 | 0.007675 | 148 | 4 | −3.0215 | 0.99998 | 0.99998 | 1 | 17934 | 0 | −3.0215 |
| TSEN54 | 4 | 1.69E−05 | 6.33E−05 | 0.007675 | 149 | 4 | −2.6133 | 0.99613 | 0.99621 | 1 | 17051 | 0 | −2.6133 |
| RPL38 | 4 | 1.74E−05 | 6.50E−05 | 0.007822 | 150 | 4 | −2.6483 | 0.99998 | 0.99998 | 1 | 17933 | 0 | −2.6483 |
| PPM1G | 4 | 1.78E−05 | 6.55E−05 | 0.007836 | 151 | 4 | −2.3909 | 0.99998 | 0.99998 | 1 | 17932 | 0 | −2.3909 |
| TRMT61A | 4 | 1.85E−05 | 6.83E−05 | 0.008057 | 152 | 3 | −3.4052 | 0.54707 | 0.69389 | 1 | 11491 | 1 | −3.4052 |
| POLR1A | 4 | 1.85E−05 | 6.83E−05 | 0.008057 | 153 | 4 | −3.1435 | 0.99998 | 0.99998 | 1 | 17931 | 0 | −3.1435 |
| IARS | 4 | 1.86E−05 | 6.88E−05 | 0.008069 | 154 | 4 | −2.5381 | 0.99998 | 0.99998 | 1 | 17930 | 0 | −2.5381 |
| GSS | 4 | 1.90E−05 | 6.99E−05 | 0.008092 | 155 | 4 | −2.073 | 0.99998 | 0.99998 | 1 | 17929 | 0 | −2.073 |
| MCL1 | 4 | 1.90E−05 | 6.99E−05 | 0.008092 | 156 | 4 | −3.5318 | 0.99998 | 0.99998 | 1 | 17928 | 0 | −3.5318 |
| SBDS | 4 | 1.91E−05 | 7.16E−05 | 0.00823 | 157 | 4 | −2.1475 | 0.99998 | 0.99998 | 1 | 17927 | 0 | −2.1475 |
| TRIT1 | 4 | 1.92E−05 | 7.21E−05 | 0.00824 | 158 | 4 | −3.1549 | 0.99998 | 0.99998 | 1 | 17926 | 0 | −3.1549 |
| PTDSS1 | 4 | 1.96E−05 | 7.27E−05 | 0.008251 | 159 | 4 | −2.5963 | 0.99998 | 0.99998 | 1 | 17925 | 0 | −2.5963 |
| POLR3C | 4 | 1.97E−05 | 7.32E−05 | 0.008261 | 160 | 4 | −1.7895 | 0.99998 | 0.99998 | 1 | 17924 | 0 | −1.7895 |
| IBA57 | 4 | 2.01E−05 | 7.49E−05 | 0.008352 | 161 | 4 | −3.6944 | 0.99996 | 0.99996 | 1 | 17876 | 0 | −3.6944 |
| MRPL45 | 4 | 2.02E−05 | 7.54E−05 | 0.008352 | 162 | 4 | −2.1855 | 0.99998 | 0.99998 | 1 | 17923 | 0 | −2.1855 |
| TSEN2 | 4 | 2.05E−05 | 7.54E−05 | 0.008352 | 163 | 4 | −2.0935 | 0.99998 | 0.99998 | 1 | 17922 | 0 | −2.0935 |
| BRD9 | 4 | 2.09E−05 | 7.71E−05 | 0.008482 | 164 | 4 | −3.23 | 0.99998 | 0.99998 | 1 | 17921 | 0 | −3.23 |
| RTEL1 | 4 | 2.14E−05 | 7.87E−05 | 0.008516 | 165 | 4 | −3.2723 | 0.99998 | 0.99998 | 1 | 17920 | 0 | −3.2723 |
| TRAPPC1 | 4 | 2.18E−05 | 7.93E−05 | 0.008516 | 166 | 4 | −3.2447 | 0.99998 | 0.99998 | 1 | 17919 | 0 | −3.2447 |
| PMM2 | 4 | 2.18E−05 | 7.93E−05 | 0.008516 | 167 | 4 | −3.387 | 0.99998 | 0.99998 | 1 | 17918 | 0 | −3.387 |
| POLE4 | 4 | 2.18E−05 | 7.93E−05 | 0.008516 | 168 | 4 | −1.8121 | 0.99395 | 0.99405 | 1 | 16900 | 0 | −1.8121 |
| WDR61 | 4 | 2.21E−05 | 8.17E−05 | 0.008663 | 169 | 4 | −2.8871 | 0.99998 | 0.99998 | 1 | 17917 | 0 | −2.8871 |
| FAM96B | 4 | 2.22E−05 | 8.20E−05 | 0.008663 | 170 | 4 | −4.3406 | 0.99992 | 0.99993 | 1 | 17807 | 0 | −4.3406 |
| STT3A | 4 | 2.24E−05 | 8.25E−05 | 0.008663 | 171 | 4 | −3.0877 | 0.99998 | 0.99998 | 1 | 17916 | 0 | −3.0877 |
| MARS | 4 | 2.24E−05 | 8.25E−05 | 0.008663 | 172 | 4 | −2.3722 | 0.99987 | 0.99988 | 1 | 17748 | 0 | −2.3722 |
| HUS1 | 4 | 2.28E−05 | 8.47E−05 | 0.008842 | 173 | 4 | −2.6423 | 0.99949 | 0.99951 | 1 | 17522 | 0 | −2.6423 |
| PGM3 | 4 | 2.29E−05 | 8.53E−05 | 0.008848 | 174 | 4 | −2.4712 | 0.99989 | 0.99989 | 1 | 17766 | 0 | −2.4712 |
| WDR7 | 4 | 2.29E−05 | 8.58E−05 | 0.008854 | 175 | 4 | −2.9691 | 0.99998 | 0.99998 | 1 | 17915 | 0 | −2.9691 |
| TOP3A | 4 | 2.36E−05 | 8.86E−05 | 0.009085 | 176 | 4 | −2.5469 | 0.99998 | 0.99998 | 1 | 17914 | 0 | −2.5469 |
| VPS29 | 4 | 2.41E−05 | 9.02E−05 | 0.009202 | 177 | 4 | −2.2629 | 0.99998 | 0.99998 | 1 | 17913 | 0 | −2.2629 |
| GLS | 4 | 2.46E−05 | 9.19E−05 | 0.009317 | 178 | 4 | −1.9743 | 0.99998 | 0.99998 | 1 | 17912 | 0 | −1.9743 |
| CTPS1 | 4 | 2.49E−05 | 9.30E−05 | 0.009323 | 179 | 4 | −3.8703 | 0.99998 | 0.99997 | 1 | 17911 | 0 | −3.8703 |
| SMAD6 | 4 | 2.50E−05 | 9.30E−05 | 0.009323 | 180 | 4 | −1.4161 | 0.99998 | 0.99997 | 1 | 17910 | 0 | −1.4161 |
| TAMM41 | 4 | 2.55E−05 | 9.41E−05 | 0.009381 | 181 | 4 | −2.5501 | 0.99978 | 0.99978 | 1 | 17673 | 0 | −2.5501 |
| WDR25 | 4 | 2.62E−05 | 9.52E−05 | 0.009439 | 182 | 4 | −2.6506 | 0.99997 | 0.99997 | 1 | 17909 | 0 | −2.6506 |
| GTPBP4 | 4 | 2.68E−05 | 9.73E−05 | 0.009553 | 183 | 4 | −2.2356 | 0.99997 | 0.99997 | 1 | 17908 | 0 | −2.2356 |
| MOGS | 4 | 2.69E−05 | 9.79E−05 | 0.009553 | 184 | 4 | −2.2311 | 0.97741 | 0.97747 | 1 | 16293 | 0 | −2.2311 |
| DRAP1 | 4 | 2.71E−05 | 9.79E−05 | 0.009553 | 185 | 4 | −2.2189 | 0.99997 | 0.99997 | 1 | 17907 | 0 | −2.2189 |
| CLTC | 4 | 2.71E−05 | 9.90E−05 | 0.009608 | 186 | 4 | −2.7205 | 0.99997 | 0.99997 | 1 | 17906 | 0 | −2.7205 |
| CCAR1 | 4 | 2.73E−05 | 0.00010009 | 0.009663 | 187 | 4 | −2.3658 | 0.99997 | 0.99997 | 1 | 17905 | 0 | −2.3658 |
| COQ4 | 4 | 2.75E−05 | 0.00010119 | 0.009717 | 188 | 4 | −3.3221 | 0.99972 | 0.99973 | 1 | 17638 | 0 | −3.3221 |
| UBE2S | 4 | 2.78E−05 | 0.00010228 | 0.009719 | 189 | 4 | −1.4783 | 0.99997 | 0.99997 | 1 | 17903 | 0 | −1.4783 |
| MRPL35 | 4 | 2.78E−05 | 0.00010228 | 0.009719 | 190 | 4 | −1.9876 | 0.99997 | 0.99997 | 1 | 17902 | 0 | −1.9876 |
| CABIN1 | 4 | 2.84E−05 | 0.00010503 | 0.009927 | 191 | 4 | −2.1162 | 0.99997 | 0.99997 | 1 | 17901 | 0 | −2.1162 |
| BUB3 | 4 | 2.87E−05 | 0.00010612 | 0.009978 | 192 | 4 | −2.7319 | 0.99997 | 0.99997 | 1 | 17900 | 0 | −2.7319 |
| CIAO1 | 4 | 2.89E−05 | 0.00010667 | 0.009978 | 193 | 4 | −2.1645 | 0.99997 | 0.99997 | 1 | 17899 | 0 | −2.1645 |
| MRPS18C | 4 | 2.90E−05 | 0.00010722 | 0.009978 | 194 | 4 | −2.2192 | 0.99997 | 0.99997 | 1 | 17898 | 0 | −2.2192 |
| DHX37 | 4 | 3.04E−05 | 0.00011051 | 0.010175 | 195 | 4 | −3.0397 | 0.99997 | 0.99997 | 1 | 17897 | 0 | −3.0397 |
| MRPS2 | 4 | 3.06E−05 | 0.00011051 | 0.010175 | 196 | 4 | −2.0941 | 0.99997 | 0.99997 | 1 | 17896 | 0 | −2.0941 |
| COX15 | 4 | 3.09E−05 | 0.00011161 | 0.010175 | 197 | 4 | −1.9335 | 0.99997 | 0.99997 | 1 | 17895 | 0 | −1.9335 |
| KNTC1 | 4 | 3.10E−05 | 0.00011161 | 0.010175 | 198 | 4 | −1.9285 | 0.99997 | 0.99997 | 1 | 17894 | 0 | −1.9285 |
| RBBP4 | 4 | 3.12E−05 | 0.00011216 | 0.010175 | 199 | 4 | −3.5793 | 0.99997 | 0.99997 | 1 | 17893 | 0 | −3.5793 |
| BRF1 | 4 | 3.21E−05 | 0.00011545 | 0.010369 | 200 | 4 | −2.323 | 0.99997 | 0.99997 | 1 | 17892 | 0 | −2.323 |
| CCDC84 | 4 | 3.21E−05 | 0.00011545 | 0.010369 | 201 | 4 | −3.0088 | 0.99997 | 0.99997 | 1 | 17891 | 0 | −3.0088 |
| CS | 4 | 3.25E−05 | 0.00011709 | 0.010465 | 202 | 4 | −2.8423 | 0.99422 | 0.99434 | 1 | 16919 | 0 | −2.8423 |
| RPN2 | 4 | 3.33E−05 | 0.00011929 | 0.010608 | 203 | 4 | −2.6274 | 0.98896 | 0.98909 | 1 | 16650 | 0 | −2.6274 |
| PRPF40A | 4 | 3.42E−05 | 0.00012422 | 0.010877 | 204 | 4 | −3.2727 | 0.99997 | 0.99997 | 1 | 17889 | 0 | −3.2727 |
| DDOST | 4 | 3.43E−05 | 0.00012532 | 0.010877 | 205 | 4 | −2.6446 | 0.99982 | 0.99983 | 1 | 17706 | 0 | −2.6446 |
| HSF1 | 4 | 3.43E−05 | 0.00012532 | 0.010877 | 206 | 4 | −1.4329 | 0.99997 | 0.99997 | 1 | 17888 | 0 | −1.4329 |
| FCHO2 | 4 | 3.44E−05 | 0.00012532 | 0.010877 | 207 | 4 | −1.7375 | 0.99997 | 0.99997 | 1 | 17887 | 0 | −1.7375 |
| VPS33B | 4 | 3.44E−05 | 0.00012532 | 0.010877 | 208 | 4 | −2.0961 | 0.99997 | 0.99997 | 1 | 17886 | 0 | −2.0961 |
| CCNC | 4 | 3.48E−05 | 0.00012751 | 0.011014 | 209 | 4 | −1.474 | 0.99997 | 0.99997 | 1 | 17885 | 0 | −1.474 |
| NELFB | 4 | 3.50E−05 | 0.00012861 | 0.011056 | 210 | 4 | −2.9271 | 0.99996 | 0.99997 | 1 | 17884 | 0 | −2.9271 |
| CSDE1 | 4 | 3.58E−05 | 0.00013135 | 0.011185 | 211 | 3 | −2.1934 | 0.95908 | 0.95896 | 1 | 15871 | 0 | −2.1934 |
| PSMG4 | 4 | 3.59E−05 | 0.00013135 | 0.011185 | 212 | 3 | −1.8249 | 0.82205 | 0.84097 | 1 | 13786 | 1 | −1.8249 |
| EIF3I | 4 | 3.72E−05 | 0.00013684 | 0.011574 | 213 | 4 | −1.9518 | 0.99996 | 0.99996 | 1 | 17883 | 0 | −1.9518 |
| USP5 | 4 | 3.73E−05 | 0.00013738 | 0.011574 | 214 | 4 | −1.6556 | 0.99412 | 0.99424 | 1 | 16914 | 0 | −1.6556 |
| RBBP5 | 4 | 3.75E−05 | 0.00013848 | 0.011574 | 215 | 4 | −1.6488 | 0.99996 | 0.99996 | 1 | 17882 | 0 | −1.6488 |
| MRPS16 | 4 | 3.76E−05 | 0.00013848 | 0.011574 | 216 | 4 | −1.7009 | 0.99996 | 0.99996 | 1 | 17881 | 0 | −1.7009 |
| RPN1 | 4 | 3.80E−05 | 0.00014067 | 0.011703 | 217 | 4 | −1.8937 | 0.99996 | 0.99996 | 1 | 17880 | 0 | −1.8937 |
| TEX10 | 4 | 3.84E−05 | 0.00014342 | 0.011877 | 218 | 4 | −2.2819 | 0.99993 | 0.99994 | 1 | 17822 | 0 | −2.2819 |
| KPNA2 | 4 | 3.88E−05 | 0.00014451 | 0.011886 | 219 | 4 | −2.254 | 0.99996 | 0.99996 | 1 | 17879 | 0 | −2.254 |
| GATC | 4 | 3.91E−05 | 0.00014561 | 0.011886 | 220 | 4 | −1.7165 | 0.99673 | 0.99677 | 1 | 17091 | 0 | −1.7165 |
| PSMD2 | 4 | 3.92E−05 | 0.00014561 | 0.011886 | 221 | 4 | −3.0147 | 0.99996 | 0.99996 | 1 | 17878 | 0 | −3.0147 |
| MRPS34 | 4 | 3.95E−05 | 0.00014616 | 0.011886 | 222 | 4 | −1.6448 | 0.99996 | 0.99996 | 1 | 17877 | 0 | −1.6448 |
| DAP3 | 4 | 3.99E−05 | 0.0001478 | 0.011966 | 223 | 4 | −1.9026 | 0.99996 | 0.99996 | 1 | 17875 | 0 | −1.9026 |
| TIMM10 | 4 | 4.01E−05 | 0.0001489 | 0.012001 | 224 | 4 | −3.4876 | 0.99992 | 0.99993 | 1 | 17808 | 0 | −3.4876 |
| MCM5 | 4 | 4.05E−05 | 0.00015137 | 0.012145 | 225 | 4 | −3.1533 | 0.99996 | 0.99996 | 1 | 17874 | 0 | −3.1533 |
| PPP2R4 | 4 | 4.07E−05 | 0.00015219 | 0.012157 | 226 | 4 | −2.3313 | 0.99996 | 0.99996 | 1 | 17873 | 0 | −2.3313 |
| FKBPL | 4 | 4.13E−05 | 0.00015329 | 0.012181 | 227 | 4 | −2.5934 | 0.99983 | 0.99985 | 1 | 17722 | 0 | −2.5934 |
| THG1L | 4 | 4.14E−05 | 0.00015384 | 0.012181 | 228 | 4 | −2.4381 | 0.9999 | 0.99991 | 1 | 17782 | 0 | −2.4381 |
| SPATA5L1 | 4 | 4.15E−05 | 0.00015493 | 0.012214 | 229 | 4 | −3.2203 | 0.98727 | 0.98736 | 1 | 16588 | 0 | −3.2203 |
| TBCB | 4 | 4.24E−05 | 0.00015877 | 0.012462 | 230 | 4 | −3.1314 | 0.99993 | 0.99994 | 1 | 17824 | 0 | −3.1314 |
| SIAH1 | 4 | 4.31E−05 | 0.00016097 | 0.012568 | 231 | 4 | −1.6227 | 0.99996 | 0.99996 | 1 | 17871 | 0 | −1.6227 |
| POLR3K | 4 | 4.35E−05 | 0.00016152 | 0.012568 | 232 | 4 | −2.0817 | 0.99996 | 0.99996 | 1 | 17870 | 0 | −2.0817 |
| EIF3F | 4 | 4.38E−05 | 0.00016234 | 0.012578 | 233 | 4 | −2.6048 | 0.99996 | 0.99996 | 1 | 17869 | 0 | −2.6048 |
| SLC25A11 | 4 | 4.41E−05 | 0.00016426 | 0.012672 | 234 | 4 | −0.98928 | 0.99996 | 0.99996 | 1 | 17868 | 0 | −0.98928 |
| DDX5 | 4 | 4.58E−05 | 0.00016645 | 0.012775 | 235 | 4 | −2.9071 | 0.99995 | 0.99996 | 1 | 17867 | 0 | −2.9071 |
| ADAT3 | 4 | 4.60E−05 | 0.000167 | 0.012775 | 236 | 4 | −2.1496 | 0.99948 | 0.9995 | 1 | 17518 | 0 | −2.1496 |
| RTN4IP1 | 4 | 4.69E−05 | 0.00017248 | 0.013139 | 237 | 3 | −2.0229 | 0.13999 | 0.28904 | 0.948895 | 5428 | 1 | −2.0229 |
| MMS19 | 4 | 4.72E−05 | 0.00017468 | 0.01325 | 238 | 4 | −1.8983 | 0.99995 | 0.99996 | 1 | 17866 | 0 | −1.8983 |
| RMI2 | 4 | 4.82E−05 | 0.00017742 | 0.013402 | 239 | 4 | −1.1562 | 0.99995 | 0.99996 | 1 | 17865 | 0 | −1.1562 |
| NOL11 | 4 | 4.87E−05 | 0.00018181 | 0.013548 | 240 | 4 | −2.7163 | 0.99995 | 0.99996 | 1 | 17864 | 0 | −2.7163 |
| EARS2 | 4 | 4.88E−05 | 0.00018236 | 0.013548 | 241 | 3 | −1.979 | 0.4518 | 0.61297 | 1 | 10290 | 1 | −1.979 |
| ZNF574 | 4 | 4.88E−05 | 0.00018236 | 0.013548 | 242 | 4 | −2.7974 | 0.99995 | 0.99996 | 1 | 17863 | 0 | −2.7974 |
| GUK1 | 4 | 4.90E−05 | 0.00018236 | 0.013548 | 243 | 4 | −2.6412 | 0.99995 | 0.99996 | 1 | 17862 | 0 | −2.6412 |
| CCT6A | 4 | 4.97E−05 | 0.00018455 | 0.013654 | 244 | 4 | −3.1654 | 0.99995 | 0.99995 | 1 | 17861 | 0 | −3.1654 |
| NHLRC2 | 4 | 5.05E−05 | 0.00018784 | 0.013825 | 245 | 4 | −2.3332 | 0.99995 | 0.99995 | 1 | 17860 | 0 | −2.3332 |
| TAF1C | 4 | 5.06E−05 | 0.00018839 | 0.013825 | 246 | 4 | −2.0676 | 0.99995 | 0.99995 | 1 | 17859 | 0 | −2.0676 |
| SHOC2 | 4 | 5.16E−05 | 0.00019168 | 0.013984 | 247 | 4 | −1.8781 | 0.99995 | 0.99995 | 1 | 17857 | 0 | −1.8781 |
| AP2S1 | 4 | 5.22E−05 | 0.00019333 | 0.013984 | 248 | 4 | −2.033 | 0.99995 | 0.99995 | 1 | 17856 | 0 | −2.033 |
| TIMELESS | 4 | 5.23E−05 | 0.00019387 | 0.013984 | 249 | 4 | −2.8123 | 0.99995 | 0.99995 | 1 | 17855 | 0 | −2.8123 |
| USP7 | 4 | 5.25E−05 | 0.00019387 | 0.013984 | 250 | 4 | −2.4245 | 0.99982 | 0.99983 | 1 | 17707 | 0 | −2.4245 |
| NUBP1 | 4 | 5.27E−05 | 0.00019442 | 0.013984 | 251 | 4 | −3.8624 | 0.99946 | 0.99948 | 1 | 17508 | 0 | −3.8624 |
| DDX11 | 4 | 5.32E−05 | 0.00019607 | 0.014013 | 252 | 4 | −2.5685 | 0.99609 | 0.99618 | 1 | 17047 | 0 | −2.5685 |
| CSNK2B | 4 | 5.35E−05 | 0.00019662 | 0.014013 | 253 | 4 | −2.5941 | 0.99995 | 0.99995 | 1 | 17854 | 0 | −2.5941 |
| MRPL47 | 4 | 5.38E−05 | 0.00019716 | 0.014013 | 254 | 4 | −3.2743 | 0.99995 | 0.99995 | 1 | 17853 | 0 | −3.2743 |
| GATAD1 | 4 | 5.51E−05 | 0.00020265 | 0.014329 | 255 | 4 | −1.7843 | 0.99994 | 0.99995 | 1 | 17852 | 0 | −1.7843 |
| RPA2 | 4 | 5.52E−05 | 0.0002032 | 0.014329 | 256 | 4 | −3.4315 | 0.99994 | 0.99995 | 1 | 17851 | 0 | −3.4315 |
| OGDH | 4 | 5.57E−05 | 0.00020484 | 0.014389 | 257 | 4 | −2.9739 | 0.99994 | 0.99995 | 1 | 17850 | 0 | −2.9739 |
| CEBPZ | 4 | 5.67E−05 | 0.00020923 | 0.014604 | 258 | 4 | −2.515 | 0.99994 | 0.99995 | 1 | 17849 | 0 | −2.515 |
| DYNC1LI2 | 4 | 5.69E−05 | 0.00021033 | 0.014604 | 259 | 4 | −1.2067 | 0.99994 | 0.99995 | 1 | 17848 | 0 | −1.2067 |
| ZFX | 4 | 5.70E−05 | 0.00021033 | 0.014604 | 260 | 4 | −1.9377 | 0.99627 | 0.99635 | 1 | 17057 | 0 | −1.9377 |
| TPI1 | 4 | 5.78E−05 | 0.00021307 | 0.014738 | 261 | 4 | −4.5939 | 0.98464 | 0.98472 | 1 | 16497 | 0 | −4.5939 |
| SMARCD1 | 4 | 5.89E−05 | 0.00021746 | 0.014984 | 262 | 4 | −1.9573 | 0.99979 | 0.99981 | 1 | 17686 | 0 | −1.9573 |
| HDAC3 | 4 | 5.94E−05 | 0.0002191 | 0.015001 | 263 | 4 | −3.0832 | 0.99994 | 0.99995 | 1 | 17846 | 0 | −3.0832 |
| CINP | 4 | 6.00E−05 | 0.00021965 | 0.015001 | 264 | 4 | −2.6506 | 0.96902 | 0.96892 | 1 | 16100 | 0 | −2.6506 |
| INTS9 | 4 | 6.01E−05 | 0.0002202 | 0.015001 | 265 | 4 | −2.5805 | 0.99994 | 0.99995 | 1 | 17845 | 0 | −2.5805 |
| WDR1 | 4 | 6.06E−05 | 0.00022239 | 0.015074 | 266 | 4 | −2.3854 | 0.99994 | 0.99995 | 1 | 17844 | 0 | −2.3854 |
| NUP50 | 4 | 6.07E−05 | 0.00022294 | 0.015074 | 267 | 4 | −1.9915 | 0.99994 | 0.99995 | 1 | 17843 | 0 | −1.9915 |
| POLR1B | 4 | 6.20E−05 | 0.00023007 | 0.015498 | 268 | 4 | −3.652 | 0.99994 | 0.99995 | 1 | 17842 | 0 | −3.652 |
| METAP1 | 4 | 6.25E−05 | 0.00023336 | 0.01564 | 269 | 4 | −2.2063 | 0.99988 | 0.99989 | 1 | 17761 | 0 | −2.2063 |
| CDK5 | 4 | 6.26E−05 | 0.00023391 | 0.01564 | 270 | 4 | −1.7612 | 0.99994 | 0.99995 | 1 | 17841 | 0 | −1.7612 |
| SNAPC3 | 4 | 6.36E−05 | 0.00023665 | 0.015758 | 271 | 4 | −2.7801 | 0.999 | 0.99898 | 1 | 17384 | 0 | −2.7801 |
| IER3IP1 | 4 | 6.39E−05 | 0.00023775 | 0.015758 | 272 | 4 | −2.3805 | 0.99965 | 0.99967 | 1 | 17598 | 0 | −2.3805 |
| ANAPC4 | 4 | 6.40E−05 | 0.0002383 | 0.015758 | 273 | 4 | −2.5454 | 0.99994 | 0.99994 | 1 | 17840 | 0 | −2.5454 |
| NDUFAF4 | 4 | 6.42E−05 | 0.00023994 | 0.015809 | 274 | 4 | −2.2946 | 0.99994 | 0.99994 | 1 | 17839 | 0 | −2.2946 |
| GOT2 | 4 | 6.45E−05 | 0.00024268 | 0.015932 | 275 | 4 | −1.6504 | 0.99986 | 0.99987 | 1 | 17741 | 0 | −1.6504 |
| TCEB2 | 4 | 6.51E−05 | 0.00024543 | 0.015995 | 276 | 4 | −4.1964 | 0.99993 | 0.99994 | 1 | 17838 | 0 | −4.1964 |
| NDNL2 | 4 | 6.53E−05 | 0.00024543 | 0.015995 | 277 | 4 | −2.6378 | 0.99921 | 0.9992 | 1 | 17429 | 0 | −2.6378 |
| VPS45 | 4 | 6.66E−05 | 0.00024872 | 0.016089 | 278 | 4 | −1.3561 | 0.99993 | 0.99994 | 1 | 17837 | 0 | −1.3561 |
| POLG2 | 4 | 6.69E−05 | 0.00025036 | 0.016089 | 279 | 4 | −3.4859 | 0.99993 | 0.99994 | 1 | 17836 | 0 | −3.4859 |
| DHX9 | 4 | 6.73E−05 | 0.00025201 | 0.016089 | 280 | 4 | −2.1098 | 0.99993 | 0.99994 | 1 | 17835 | 0 | −2.1098 |
| RPL14 | 4 | 6.74E−05 | 0.00025201 | 0.016089 | 281 | 4 | −2.3474 | 0.99993 | 0.99994 | 1 | 17834 | 0 | −2.3474 |
| SUPT3H | 4 | 6.76E−05 | 0.00025256 | 0.016089 | 282 | 4 | −2.5261 | 0.99993 | 0.99994 | 1 | 17833 | 0 | −2.5261 |
| CD3EAP | 4 | 6.78E−05 | 0.00025311 | 0.016089 | 283 | 4 | −1.5945 | 0.99744 | 0.99748 | 1 | 17165 | 0 | −1.5945 |
| VBP1 | 4 | 6.79E−05 | 0.00025311 | 0.016089 | 284 | 4 | −1.9275 | 0.99993 | 0.99994 | 1 | 17832 | 0 | −1.9275 |
| NXT1 | 4 | 6.85E−05 | 0.00025585 | 0.016206 | 285 | 4 | −1.8937 | 0.99993 | 0.99994 | 1 | 17831 | 0 | −1.8937 |
| RARS2 | 4 | 6.94E−05 | 0.00025914 | 0.016357 | 286 | 4 | −2.6832 | 0.99993 | 0.99994 | 1 | 17829 | 0 | −2.6832 |
| CENPM | 4 | 7.00E−05 | 0.00026023 | 0.016369 | 287 | 4 | −2.4904 | 0.99993 | 0.99994 | 1 | 17828 | 0 | −2.4904 |
| MRP63 | 4 | 7.05E−05 | 0.00026298 | 0.016383 | 288 | 4 | −3.6504 | 0.99993 | 0.99994 | 1 | 17827 | 0 | −3.6504 |
| SOD1 | 4 | 7.06E−05 | 0.00026298 | 0.016383 | 289 | 3 | −4.3842 | 0.86104 | 0.86726 | 1 | 14237 | 1 | −4.3842 |
| TUBG1 | 4 | 7.09E−05 | 0.00026407 | 0.016383 | 290 | 4 | −1.4805 | 0.99993 | 0.99994 | 1 | 17826 | 0 | −1.4805 |
| AIFM1 | 4 | 7.12E−05 | 0.00026407 | 0.016383 | 291 | 4 | −2.0548 | 0.99993 | 0.99994 | 1 | 17825 | 0 | −2.0548 |
| TTC4 | 4 | 7.25E−05 | 0.00026736 | 0.016408 | 292 | 4 | −1.3964 | 0.99993 | 0.99994 | 1 | 17823 | 0 | −1.3964 |
| DNAJC17 | 4 | 7.30E−05 | 0.00026791 | 0.016408 | 293 | 4 | −2.2686 | 0.99993 | 0.99994 | 1 | 17821 | 0 | −2.2686 |
| BANF1 | 4 | 7.31E−05 | 0.00026791 | 0.016408 | 294 | 4 | −3.0135 | 0.99993 | 0.99993 | 1 | 17820 | 0 | −3.0135 |
| ATP5O | 4 | 7.34E−05 | 0.00027011 | 0.016408 | 295 | 4 | −3.3124 | 0.99702 | 0.99706 | 1 | 17117 | 0 | −3.3124 |
| PSMG1 | 4 | 7.35E−05 | 0.00027011 | 0.016408 | 296 | 4 | −2.261 | 0.99993 | 0.99993 | 1 | 17819 | 0 | −2.261 |
| POP5 | 4 | 7.36E−05 | 0.00027066 | 0.016408 | 297 | 4 | −1.9812 | 0.99993 | 0.99993 | 1 | 17818 | 0 | −1.9812 |
| AURKAIP1 | 4 | 7.38E−05 | 0.0002712 | 0.016408 | 298 | 4 | −1.0891 | 0.99993 | 0.99993 | 1 | 17817 | 0 | −1.0891 |
| NOP16 | 4 | 7.38E−05 | 0.00027175 | 0.016408 | 299 | 4 | −1.8504 | 0.99993 | 0.99993 | 1 | 17816 | 0 | −1.8504 |
| SLC35B1 | 4 | 7.50E−05 | 0.00027943 | 0.016815 | 300 | 4 | −2.6342 | 0.99993 | 0.99993 | 1 | 17815 | 0 | −2.6342 |
| CPSF4 | 4 | 7.58E−05 | 0.00028217 | 0.016924 | 301 | 4 | −1.9698 | 0.99992 | 0.99993 | 1 | 17814 | 0 | −1.9698 |
| NELFA | 4 | 7.65E−05 | 0.00028437 | 0.016952 | 302 | 4 | −2.1041 | 0.99992 | 0.99993 | 1 | 17813 | 0 | −2.1041 |
| RPP21 | 4 | 7.66E−05 | 0.00028491 | 0.016952 | 303 | 4 | −2.4666 | 0.99403 | 0.99415 | 1 | 16906 | 0 | −2.4666 |
| CHTF8 | 4 | 7.69E−05 | 0.00028546 | 0.016952 | 304 | 4 | −2.1497 | 0.99992 | 0.99993 | 1 | 17812 | 0 | −2.1497 |
| GTPBP8 | 4 | 7.74E−05 | 0.00028821 | 0.017059 | 305 | 4 | −2.6554 | 0.99992 | 0.99993 | 1 | 17811 | 0 | −2.6554 |
| LCMT1 | 4 | 7.79E−05 | 0.00029095 | 0.017165 | 306 | 4 | −2.3379 | 0.99992 | 0.99993 | 1 | 17810 | 0 | −2.3379 |
| TXNL4A | 4 | 7.83E−05 | 0.00029204 | 0.017174 | 307 | 4 | −3.6409 | 0.99992 | 0.99993 | 1 | 17809 | 0 | −3.6409 |
| PIM3 | 4 | 7.96E−05 | 0.0002978 | 0.017455 | 308 | 4 | −1.3672 | 0.99992 | 0.99993 | 1 | 17806 | 0 | −1.3672 |
| ORAOV1 | 4 | 8.06E−05 | 0.00030246 | 0.017614 | 309 | 4 | −3.2885 | 0.9998 | 0.99981 | 1 | 17688 | 0 | −3.2885 |
| RFK | 4 | 8.19E−05 | 0.0003063 | 0.017714 | 310 | 4 | −2.7048 | 0.99921 | 0.9992 | 1 | 17430 | 0 | −2.7048 |
| DDX21 | 4 | 8.21E−05 | 0.00030685 | 0.017714 | 311 | 4 | −2.5662 | 0.99992 | 0.99992 | 1 | 17805 | 0 | −2.5662 |
| HSPA14 | 4 | 8.23E−05 | 0.00030713 | 0.017714 | 312 | 4 | −5.062 | 0.99992 | 0.99992 | 1 | 17804 | 0 | −5.062 |
| VMP1 | 4 | 8.51E−05 | 0.00031453 | 0.017788 | 313 | 3 | −2.6897 | 0.87315 | 0.87655 | 1 | 14404 | 1 | −2.6897 |
| CPSF3L | 4 | 8.54E−05 | 0.00031508 | 0.017788 | 314 | 4 | −2.6193 | 0.99991 | 0.99992 | 1 | 17803 | 0 | −2.6193 |
| DAXX | 4 | 8.55E−05 | 0.00031508 | 0.017788 | 315 | 4 | −2.4364 | 0.99991 | 0.99992 | 1 | 17802 | 0 | −2.4364 |
| EXOSC5 | 4 | 8.55E−05 | 0.00031508 | 0.017788 | 316 | 4 | −2.7212 | 0.99991 | 0.99992 | 1 | 17801 | 0 | −2.7212 |
| TRAPPC4 | 4 | 8.57E−05 | 0.00031563 | 0.017788 | 317 | 3 | −2.0056 | 0.83326 | 0.84799 | 1 | 13911 | 1 | −2.0056 |
| STAT3 | 4 | 8.58E−05 | 0.00031672 | 0.017788 | 318 | 4 | −2.1961 | 0.99991 | 0.99992 | 1 | 17800 | 0 | −2.1961 |
| TONSL | 4 | 8.59E−05 | 0.00031672 | 0.017788 | 319 | 4 | −1.9853 | 0.99991 | 0.99992 | 1 | 17799 | 0 | −1.9853 |
| PDAP1 | 4 | 8.61E−05 | 0.00031727 | 0.017788 | 320 | 4 | −2.9338 | 0.99991 | 0.99992 | 1 | 17798 | 0 | −2.9338 |
| DDX46 | 4 | 8.62E−05 | 0.00031727 | 0.017788 | 321 | 4 | −3.369 | 0.99991 | 0.99992 | 1 | 17797 | 0 | −3.369 |
| RABGGTA | 4 | 8.69E−05 | 0.00031947 | 0.017837 | 322 | 4 | −1.9715 | 0.99991 | 0.99992 | 1 | 17796 | 0 | −1.9715 |
| GFM2 | 4 | 8.77E−05 | 0.00032056 | 0.017837 | 323 | 4 | −2.1147 | 0.99991 | 0.99992 | 1 | 17795 | 0 | −2.1147 |
| PSMA5 | 4 | 8.78E−05 | 0.00032111 | 0.017837 | 324 | 4 | −2.654 | 0.99991 | 0.99992 | 1 | 17794 | 0 | −2.654 |
| PSMG3 | 4 | 8.83E−05 | 0.00032385 | 0.01791 | 325 | 4 | −3.1463 | 0.99991 | 0.99992 | 1 | 17793 | 0 | −3.1463 |
| PAFAH1B1 | 4 | 8.84E−05 | 0.0003244 | 0.01791 | 326 | 4 | −2.3477 | 0.99991 | 0.99992 | 1 | 17792 | 0 | −2.3477 |
| DDX56 | 4 | 8.93E−05 | 0.00032714 | 0.018006 | 327 | 4 | −1.5513 | 0.99991 | 0.99992 | 1 | 17791 | 0 | −1.5513 |
| NDOR1 | 4 | 8.97E−05 | 0.00032906 | 0.018057 | 328 | 4 | −3.3717 | 0.99991 | 0.99992 | 1 | 17790 | 0 | −3.3717 |
| MRPL37 | 4 | 9.08E−05 | 0.00033537 | 0.018241 | 329 | 4 | −1.826 | 0.99953 | 0.99955 | 1 | 17534 | 0 | −1.826 |
| HNRNPL | 4 | 9.10E−05 | 0.00033592 | 0.018241 | 330 | 4 | −2.7067 | 0.99991 | 0.99992 | 1 | 17789 | 0 | −2.7067 |
| TEFM | 4 | 9.11E−05 | 0.00033592 | 0.018241 | 331 | 4 | −1.7986 | 0.98801 | 0.9881 | 1 | 16614 | 0 | −1.7986 |
| NOP9 | 4 | 9.12E−05 | 0.00033647 | 0.018241 | 332 | 4 | −2.0625 | 0.99938 | 0.9994 | 1 | 17481 | 0 | −2.0625 |
| RAB14 | 4 | 9.21E−05 | 0.00033976 | 0.018364 | 333 | 4 | −0.95936 | 0.99991 | 0.99991 | 1 | 17788 | 0 | −0.95936 |
| NPC1 | 4 | 9.26E−05 | 0.0003414 | 0.018398 | 334 | 4 | −1.6889 | 0.99991 | 0.99991 | 1 | 17787 | 0 | −1.6889 |
| DET1 | 4 | 9.34E−05 | 0.0003436 | 0.018461 | 335 | 4 | −2.0347 | 0.96744 | 0.96734 | 1 | 16062 | 0 | −2.0347 |
| ANAPC13 | 4 | 9.48E−05 | 0.00035073 | 0.018762 | 336 | 4 | −1.6171 | 0.99991 | 0.99991 | 1 | 17786 | 0 | −1.6171 |
| DHODH | 4 | 9.49E−05 | 0.00035128 | 0.018762 | 337 | 4 | −1.431 | 0.99991 | 0.99991 | 1 | 17785 | 0 | −1.431 |
| ELP3 | 4 | 9.55E−05 | 0.00035347 | 0.018824 | 338 | 4 | −1.6986 | 0.9999 | 0.99991 | 1 | 17784 | 0 | −1.6986 |
| CDC16 | 4 | 9.57E−05 | 0.00035457 | 0.018826 | 339 | 4 | −3.6391 | 0.9999 | 0.99991 | 1 | 17783 | 0 | −3.6391 |
| SNAPC4 | 4 | 9.61E−05 | 0.00035566 | 0.018829 | 340 | 3 | −2.7157 | 0.48844 | 0.64388 | 1 | 10736 | 1 | −2.7157 |
| TACC3 | 4 | 9.79E−05 | 0.00036554 | 0.019164 | 341 | 4 | −2.7861 | 0.9998 | 0.99981 | 1 | 17692 | 0 | −2.7861 |
| EEF1A1 | 3 | 9.81E−05 | 0.00030027 | 0.017543 | 342 | 3 | −1.8285 | 0.9999 | 0.9999 | 1 | 17781 | 0 | −1.8285 |
| NARG2 | 4 | 9.83E−05 | 0.00036608 | 0.019164 | 343 | 4 | −1.9078 | 0.9999 | 0.99991 | 1 | 17780 | 0 | −1.9078 |
| PDSS2 | 4 | 9.88E−05 | 0.00036718 | 0.019164 | 344 | 4 | −2.8876 | 0.9999 | 0.99991 | 1 | 17779 | 0 | −2.8876 |
| MED8 | 4 | 9.93E−05 | 0.00036773 | 0.019164 | 345 | 4 | −1.7707 | 0.9999 | 0.99991 | 1 | 17778 | 0 | −1.7707 |
| RFT1 | 4 | 9.98E−05 | 0.0003702 | 0.019164 | 346 | 4 | −2.2279 | 0.9999 | 0.99991 | 1 | 17777 | 0 | −2.2279 |
| PCBP1 | 4 | 9.99E−05 | 0.00037047 | 0.019164 | 347 | 4 | −3.4803 | 0.99984 | 0.99985 | 1 | 17727 | 0 | −3.4803 |
| ACTR6 | 4 | 9.99E−05 | 0.00037047 | 0.019164 | 348 | 4 | −2.2682 | 0.9999 | 0.99991 | 1 | 17776 | 0 | −2.2682 |
| UBIAD1 | 4 | 0.00010078 | 0.00037321 | 0.019227 | 349 | 4 | −2.4309 | 0.99943 | 0.99945 | 1 | 17498 | 0 | −2.4309 |
| ELP2 | 4 | 0.00010129 | 0.00037541 | 0.019227 | 350 | 4 | −1.5379 | 0.9999 | 0.9999 | 1 | 17775 | 0 | −1.5379 |
| ISG20L2 | 4 | 0.00010135 | 0.00037541 | 0.019227 | 351 | 4 | −3.2285 | 0.9727 | 0.97266 | 1 | 16184 | 0 | −3.2285 |
| NARFL | 4 | 0.00010152 | 0.00037596 | 0.019227 | 352 | 4 | −2.0631 | 0.9999 | 0.9999 | 1 | 17774 | 0 | −2.0631 |
| TOE1 | 4 | 0.00010171 | 0.00037705 | 0.019229 | 353 | 4 | −2.2785 | 0.99731 | 0.99733 | 1 | 17143 | 0 | −2.2785 |
| COPS8 | 4 | 0.00010241 | 0.00037979 | 0.019261 | 354 | 3 | −1.4987 | 0.95356 | 0.95341 | 1 | 15769 | 0 | −1.4987 |
| CCT4 | 4 | 0.00010261 | 0.00038089 | 0.019261 | 355 | 4 | −3.0286 | 0.9999 | 0.9999 | 1 | 17773 | 0 | −3.0286 |
| OIP5 | 4 | 0.00010265 | 0.00038089 | 0.019261 | 356 | 3 | −2.5251 | 0.9479 | 0.94781 | 1 | 15656 | 0 | −2.5251 |
| UBA3 | 4 | 0.00010323 | 0.00038363 | 0.019346 | 357 | 4 | −2.1471 | 0.99686 | 0.9969 | 1 | 17103 | 0 | −2.1471 |
| CNPY2 | 4 | 0.00010394 | 0.00038528 | 0.019375 | 358 | 4 | −1.571 | 0.9999 | 0.9999 | 1 | 17772 | 0 | −1.571 |
| DENR | 4 | 0.000107 | 0.00039899 | 0.020008 | 359 | 4 | −1.7642 | 0.99989 | 0.9999 | 1 | 17771 | 0 | −1.7642 |
| BRAP | 4 | 0.00010831 | 0.00040173 | 0.02009 | 360 | 4 | −2.0494 | 0.99989 | 0.99989 | 1 | 17770 | 0 | −2.0494 |
| PPP2CA | 4 | 0.00010933 | 0.00040393 | 0.020116 | 361 | 4 | −2.4907 | 0.99989 | 0.99989 | 1 | 17769 | 0 | −2.4907 |
| C19orf53 | 4 | 0.00010957 | 0.00040447 | 0.020116 | 362 | 4 | −2.8933 | 0.99989 | 0.99989 | 1 | 17768 | 0 | −2.8933 |
| YAE1D1 | 4 | 0.00011039 | 0.00040722 | 0.020168 | 363 | 3 | −2.3644 | 0.91483 | 0.91462 | 1 | 15030 | 0 | −2.3644 |
| UGP2 | 4 | 0.00011069 | 0.00040777 | 0.020168 | 364 | 4 | −1.9954 | 0.99726 | 0.99729 | 1 | 17140 | 0 | −1.9954 |
| MRPL21 | 4 | 0.00011134 | 0.00041051 | 0.020248 | 365 | 4 | −1.9138 | 0.99989 | 0.99989 | 1 | 17767 | 0 | −1.9138 |
| VHL | 4 | 0.00011222 | 0.00041599 | 0.020434 | 366 | 4 | −4.2785 | 0.99518 | 0.99526 | 1 | 16985 | 0 | −4.2785 |
| CENPN | 4 | 0.00011238 | 0.00041654 | 0.020434 | 367 | 4 | −4.193 | 0.99989 | 0.99989 | 1 | 17765 | 0 | −4.193 |
| WDR77 | 4 | 0.00011311 | 0.00042093 | 0.020589 | 368 | 4 | −2.827 | 0.99856 | 0.99858 | 1 | 17297 | 0 | −2.827 |
| ARID4B | 4 | 0.00011356 | 0.00042202 | 0.020589 | 369 | 4 | −1.7198 | 0.99989 | 0.99989 | 1 | 17764 | 0 | −1.7198 |
| APEX1 | 4 | 0.00011384 | 0.00042312 | 0.020589 | 370 | 4 | −2.0917 | 0.9998 | 0.99981 | 1 | 17690 | 0 | −2.0917 |
| NDUFB9 | 4 | 0.00011443 | 0.00042751 | 0.020747 | 371 | 4 | −1.8927 | 0.99989 | 0.99989 | 1 | 17763 | 0 | −1.8927 |
| MCMBP | 4 | 0.00011468 | 0.00042998 | 0.020768 | 372 | 4 | −3.0224 | 0.99989 | 0.99989 | 1 | 17762 | 0 | −3.0224 |
| OPA1 | 4 | 0.0001147 | 0.00043025 | 0.020768 | 373 | 4 | −1.5664 | 0.97167 | 0.97161 | 1 | 16158 | 0 | −1.5664 |
| PRKRIR | 3 | 0.00011602 | 0.00036334 | 0.019164 | 374 | 3 | −2.4693 | 0.99988 | 0.99987 | 1 | 17758 | 0 | −2.4693 |
| MRPL34 | 4 | 0.00011733 | 0.00044122 | 0.021241 | 375 | 4 | −2.7561 | 0.99988 | 0.99989 | 1 | 17760 | 0 | −2.7561 |
| MYBL2 | 4 | 0.0001188 | 0.00045 | 0.021606 | 376 | 4 | −1.2351 | 0.99988 | 0.99989 | 1 | 17759 | 0 | −1.2351 |
| C12orf45 | 4 | 0.00012094 | 0.00045932 | 0.021952 | 377 | 4 | −1.7613 | 0.99988 | 0.99988 | 1 | 17757 | 0 | −1.7613 |
| TOP2A | 4 | 0.0001212 | 0.00046042 | 0.021952 | 378 | 4 | −2.553 | 0.99988 | 0.99988 | 1 | 17756 | 0 | −2.553 |
| XPR1 | 4 | 0.00012139 | 0.00046151 | 0.021952 | 379 | 4 | −2.6557 | 0.99988 | 0.99988 | 1 | 17755 | 0 | −2.6557 |
| PAPOLA | 4 | 0.00012152 | 0.00046206 | 0.021952 | 380 | 4 | −2.7333 | 0.99988 | 0.99988 | 1 | 17754 | 0 | −2.7333 |
| RPTOR | 4 | 0.00012277 | 0.00046535 | 0.022018 | 381 | 4 | −1.491 | 0.99988 | 0.99988 | 1 | 17753 | 0 | −1.491 |
| GGPS1 | 4 | 0.0001231 | 0.0004659 | 0.022018 | 382 | 4 | −2.4365 | 0.99988 | 0.99988 | 1 | 17752 | 0 | −2.4365 |
| IL6ST | 4 | 0.00012402 | 0.00047029 | 0.022135 | 383 | 4 | −1.9144 | 0.99988 | 0.99988 | 1 | 17751 | 0 | −1.9144 |
| COASY | 4 | 0.00012433 | 0.00047084 | 0.022135 | 384 | 4 | −2.1957 | 0.99836 | 0.99837 | 1 | 17265 | 0 | −2.1957 |
| MRPL28 | 4 | 0.00012462 | 0.00047303 | 0.022181 | 385 | 4 | −2.504 | 0.99988 | 0.99988 | 1 | 17750 | 0 | −2.504 |
| TNPO3 | 4 | 0.00012502 | 0.00047577 | 0.02222 | 386 | 4 | −1.9706 | 0.99987 | 0.99988 | 1 | 17749 | 0 | −1.9706 |
| AMBRA1 | 4 | 0.00012511 | 0.00047632 | 0.02222 | 387 | 4 | −1.4323 | 0.99885 | 0.99884 | 1 | 17357 | 0 | −1.4323 |
| KIAAO020 | 4 | 0.00012665 | 0.0004818 | 0.022418 | 388 | 4 | −2.7757 | 0.99971 | 0.99972 | 1 | 17632 | 0 | −2.7757 |
| NCAPG | 4 | 0.00013138 | 0.00050429 | 0.023403 | 389 | 3 | −2.8034 | 0.95282 | 0.95266 | 1 | 15756 | 0 | −2.8034 |
| HMBS | 4 | 0.00013212 | 0.00050813 | 0.023486 | 390 | 3 | −2.2962 | 0.92159 | 0.92138 | 1 | 15151 | 0 | −2.2962 |
| NPLOC4 | 4 | 0.00013218 | 0.00050868 | 0.023486 | 391 | 4 | −1.6636 | 0.99987 | 0.99987 | 1 | 17747 | 0 | −1.6636 |
| NLE1 | 4 | 0.00013288 | 0.00051087 | 0.023493 | 392 | 4 | −2.122 | 0.99987 | 0.99987 | 1 | 17746 | 0 | −2.122 |
| MED25 | 4 | 0.00013302 | 0.00051142 | 0.023493 | 393 | 4 | −3.234 | 0.99987 | 0.99987 | 1 | 17745 | 0 | −3.234 |
| WNK1 | 4 | 0.00013518 | 0.0005202 | 0.023835 | 394 | 4 | −2.7505 | 0.99791 | 0.99795 | 1 | 17208 | 0 | −2.7505 |
| POLR3B | 4 | 0.00013662 | 0.00052403 | 0.023919 | 395 | 4 | −3.0987 | 0.99986 | 0.99987 | 1 | 17743 | 0 | −3.0987 |
| TSR2 | 4 | 0.00013734 | 0.00052568 | 0.023919 | 396 | 4 | −1.6642 | 0.99986 | 0.99987 | 1 | 17742 | 0 | −1.6642 |
| BIRC6 | 4 | 0.00013784 | 0.00052623 | 0.023919 | 397 | 4 | −2.9481 | 0.99986 | 0.99987 | 1 | 17740 | 0 | −2.9481 |
| ABT1 | 4 | 0.00013848 | 0.00052733 | 0.023919 | 398 | 4 | −1.6801 | 0.99986 | 0.99987 | 1 | 17739 | 0 | −1.6801 |
| PPP4C | 4 | 0.00014047 | 0.00053391 | 0.024157 | 399 | 4 | −2.7617 | 0.99856 | 0.99858 | 1 | 17299 | 0 | −2.7617 |
| DYRK1A | 4 | 0.00014114 | 0.00053665 | 0.02422 | 400 | 4 | −1.4627 | 0.99948 | 0.9995 | 1 | 17516 | 0 | −1.4627 |
| C10orf2 | 4 | 0.00014352 | 0.00054707 | 0.024629 | 401 | 4 | −3.0708 | 0.99837 | 0.9984 | 1 | 17270 | 0 | −3.0708 |
| STIL | 4 | 0.00014419 | 0.00054981 | 0.024691 | 402 | 4 | −1.9093 | 0.99986 | 0.99987 | 1 | 17738 | 0 | −1.9093 |
| POT1 | 4 | 0.00014764 | 0.00056188 | 0.025119 | 403 | 4 | −1.7346 | 0.99985 | 0.99986 | 1 | 17737 | 0 | −1.7346 |
| RRP9 | 4 | 0.00014773 | 0.00056243 | 0.025119 | 404 | 3 | −1.9825 | 0.76328 | 0.80864 | 1 | 13250 | 1 | −1.9825 |
| PAICS | 4 | 0.00014794 | 0.00056352 | 0.025119 | 405 | 4 | −2.1179 | 0.99985 | 0.99986 | 1 | 17736 | 0 | −2.1179 |
| FARSB | 4 | 0.00015006 | 0.0005712 | 0.025344 | 406 | 4 | −2.3882 | 0.99877 | 0.99876 | 1 | 17338 | 0 | −2.3882 |
| BOP1 | 4 | 0.00015077 | 0.00057394 | 0.025344 | 407 | 4 | −1.8936 | 0.99985 | 0.99986 | 1 | 17735 | 0 | −1.8936 |
| POLR2L | 4 | 0.00015084 | 0.00057449 | 0.025344 | 408 | 4 | −2.6582 | 0.99985 | 0.99986 | 1 | 17734 | 0 | −2.6582 |
| SNF8 | 4 | 0.00015089 | 0.00057504 | 0.025344 | 409 | 3 | −2.4308 | 0.50025 | 0.65376 | 1 | 10877 | 1 | −2.4308 |
| EXT2 | 4 | 0.0001513 | 0.00057778 | 0.025379 | 410 | 4 | −0.9659 | 0.99985 | 0.99986 | 1 | 17733 | 0 | −0.9659 |
| PSMB2 | 4 | 0.00015316 | 0.00058656 | 0.025702 | 411 | 4 | −2.4014 | 0.99985 | 0.99986 | 1 | 17732 | 0 | −2.4014 |
| TEAD1 | 4 | 0.00015433 | 0.00059149 | 0.025817 | 412 | 4 | −1.2851 | 0.99985 | 0.99986 | 1 | 17731 | 0 | −1.2851 |
| PPIL4 | 4 | 0.00015448 | 0.00059204 | 0.025817 | 413 | 4 | −2.4198 | 0.98414 | 0.9842 | 1 | 16474 | 0 | −2.4198 |
| SRP72 | 4 | 0.00015543 | 0.00059478 | 0.025843 | 414 | 4 | −1.5856 | 0.99984 | 0.99986 | 1 | 17730 | 0 | −1.5856 |
| SDHC | 4 | 0.00015629 | 0.00059807 | 0.025843 | 415 | 4 | −1.4111 | 0.99984 | 0.99986 | 1 | 17729 | 0 | −1.4111 |
| CENPW | 4 | 0.00015677 | 0.00059862 | 0.025843 | 416 | 4 | −2.515 | 0.99984 | 0.99986 | 1 | 17728 | 0 | −2.515 |
| GFPT1 | 4 | 0.00015773 | 0.00060136 | 0.025843 | 417 | 3 | −2.4517 | 0.95031 | 0.95021 | 1 | 15700 | 0 | −2.4517 |
| ZC3H11A | 4 | 0.00015828 | 0.00060136 | 0.025843 | 418 | 4 | −1.682 | 0.99984 | 0.99985 | 1 | 17726 | 0 | −1.682 |
| ACTR1A | 4 | 0.0001587 | 0.00060246 | 0.025843 | 419 | 3 | −2.6528 | 0.4607 | 0.62048 | 1 | 10390 | 1 | −2.6528 |
| VPS18 | 4 | 0.00015924 | 0.00060356 | 0.025843 | 420 | 4 | −1.7708 | 0.99984 | 0.99985 | 1 | 17725 | 0 | −1.7708 |
| MRPL38 | 4 | 0.00015946 | 0.00060411 | 0.025843 | 421 | 3 | −2.3802 | 0.9597 | 0.95958 | 1 | 15886 | 0 | −2.3802 |
| RNASEH2B | 4 | 0.00016103 | 0.00061069 | 0.026063 | 422 | 3 | −2.5787 | 0.34759 | 0.52544 | 1 | 9078 | 1 | −2.5787 |
| DDX52 | 4 | 0.00016352 | 0.00062166 | 0.026469 | 423 | 4 | −2.0537 | 0.99984 | 0.99985 | 1 | 17724 | 0 | −2.0537 |
| COPS5 | 4 | 0.00016499 | 0.00062604 | 0.026529 | 424 | 4 | −1.8321 | 0.99984 | 0.99985 | 1 | 17723 | 0 | −1.8321 |
| TAF7 | 4 | 0.00016623 | 0.00062934 | 0.026529 | 425 | 4 | −1.9217 | 0.99983 | 0.99985 | 1 | 17721 | 0 | −1.9217 |
| CCT7 | 4 | 0.00016631 | 0.00062934 | 0.026529 | 426 | 4 | −2.7928 | 0.99983 | 0.99985 | 1 | 17720 | 0 | −2.7928 |
| CCT3 | 4 | 0.00016648 | 0.00063043 | 0.026529 | 427 | 4 | −2.1157 | 0.99983 | 0.99985 | 1 | 17719 | 0 | −2.1157 |
| GEMIN4 | 4 | 0.00016677 | 0.00063153 | 0.026529 | 428 | 3 | −1.988 | 0.27512 | 0.46546 | 1 | 8213 | 1 | −1.988 |
| NARS | 4 | 0.00016756 | 0.00063482 | 0.026529 | 429 | 4 | −3.1441 | 0.99983 | 0.99984 | 1 | 17718 | 0 | −3.1441 |
| ARHGAP1 | 4 | 0.00016764 | 0.00063482 | 0.026529 | 430 | 4 | −1.4013 | 0.99983 | 0.99984 | 1 | 17717 | 0 | −1.4013 |
| SLC25A13 | 4 | 0.00016777 | 0.00063482 | 0.026529 | 431 | 4 | −1.4577 | 0.99955 | 0.99957 | 1 | 17539 | 0 | −1.4577 |
| CCNK | 4 | 0.00017049 | 0.00064524 | 0.026863 | 432 | 4 | −2.6671 | 0.99983 | 0.99984 | 1 | 17715 | 0 | −2.6671 |
| ATP6V1D | 4 | 0.00017099 | 0.00064579 | 0.026863 | 433 | 4 | −3.8295 | 0.99983 | 0.99984 | 1 | 17714 | 0 | −3.8295 |
| UBA2 | 4 | 0.00017184 | 0.00065072 | 0.027002 | 434 | 4 | −1.2153 | 0.99983 | 0.99984 | 1 | 17713 | 0 | −1.2153 |
| NOL9 | 4 | 0.00017294 | 0.00065292 | 0.027002 | 435 | 4 | −1.6659 | 0.99983 | 0.99984 | 1 | 17712 | 0 | −1.6659 |
| POLRMT | 4 | 0.00017362 | 0.00065511 | 0.027002 | 436 | 4 | −1.8619 | 0.99983 | 0.99984 | 1 | 17711 | 0 | −1.8619 |
| PRMT5 | 4 | 0.00017366 | 0.00065511 | 0.027002 | 437 | 4 | −2.0145 | 0.99981 | 0.99982 | 1 | 17700 | 0 | −2.0145 |
| GRPEL1 | 4 | 0.00017422 | 0.00065731 | 0.02703 | 438 | 4 | −3.2639 | 0.99983 | 0.99984 | 1 | 17710 | 0 | −3.2639 |
| CMPK1 | 4 | 0.0001749 | 0.0006595 | 0.027059 | 439 | 4 | −2.5332 | 0.98974 | 0.98983 | 1 | 16681 | 0 | −2.5332 |
| TTI1 | 4 | 0.00017828 | 0.00067156 | 0.027492 | 440 | 4 | −1.5958 | 0.99982 | 0.99983 | 1 | 17709 | 0 | −1.5958 |
| HECTD1 | 4 | 0.00017907 | 0.00067431 | 0.027541 | 441 | 4 | −2.9661 | 0.99982 | 0.99983 | 1 | 17708 | 0 | −2.9661 |
| SMC4 | 4 | 0.0001817 | 0.00068418 | 0.027881 | 442 | 4 | −2.2226 | 0.99982 | 0.99983 | 1 | 17705 | 0 | −2.2226 |
| AP2B1 | 4 | 0.00018206 | 0.00068637 | 0.027908 | 443 | 4 | −1.7461 | 0.99982 | 0.99983 | 1 | 17704 | 0 | −1.7461 |
| PPP2R2A | 4 | 0.00018383 | 0.0006935 | 0.028134 | 444 | 4 | −2.9113 | 0.99982 | 0.99982 | 1 | 17703 | 0 | −2.9113 |
| EXOSC4 | 4 | 0.00018455 | 0.00069734 | 0.028227 | 445 | 4 | −2.4784 | 0.99982 | 0.99982 | 1 | 17702 | 0 | −2.4784 |
| UBA1 | 4 | 0.00018607 | 0.00069954 | 0.028252 | 446 | 4 | −2.7021 | 0.998 | 0.99803 | 1 | 17220 | 0 | −2.7021 |
| SPATA5 | 4 | 0.00018697 | 0.00070392 | 0.028293 | 447 | 4 | −1.5036 | 0.99981 | 0.99982 | 1 | 17701 | 0 | −1.5036 |
| MCM2 | 4 | 0.00018714 | 0.00070392 | 0.028293 | 448 | 4 | −3.1096 | 0.99963 | 0.99964 | 1 | 17585 | 0 | −3.1096 |
| MRPS12 | 4 | 0.00018779 | 0.00070721 | 0.028293 | 449 | 3 | −1.9604 | 0.68589 | 0.77401 | 1 | 12728 | 1 | −1.9604 |
| EIF4E | 4 | 0.00018823 | 0.00070886 | 0.028293 | 450 | 4 | −2.467 | 0.99968 | 0.9997 | 1 | 17612 | 0 | −2.467 |
| TRNAU1AP | 4 | 0.0001886 | 0.00070941 | 0.028293 | 451 | 4 | −2.0707 | 0.99981 | 0.99982 | 1 | 17699 | 0 | −2.0707 |
| C21orf59 | 4 | 0.00018897 | 0.00070996 | 0.028293 | 452 | 4 | −2.7242 | 0.99981 | 0.99982 | 1 | 17698 | 0 | −2.7242 |
| GTF2F2 | 3 | 0.00018966 | 0.00057559 | 0.025344 | 453 | 3 | −2.2508 | 0.99981 | 0.99981 | 1 | 17697 | 0 | −2.2508 |
| MGEA5 | 4 | 0.00019291 | 0.00072367 | 0.028735 | 454 | 4 | −1.7108 | 0.99981 | 0.99982 | 1 | 17696 | 0 | −1.7108 |
| OSTC | 4 | 0.00019319 | 0.00072422 | 0.028735 | 455 | 4 | −1.6079 | 0.99981 | 0.99982 | 1 | 17695 | 0 | −1.6079 |
| RNF4 | 4 | 0.00019504 | 0.00073464 | 0.029042 | 456 | 4 | −1.7706 | 0.9998 | 0.99981 | 1 | 17694 | 0 | −1.7706 |
| ZNRD1 | 4 | 0.00019532 | 0.00073518 | 0.029042 | 457 | 4 | −1.4721 | 0.9998 | 0.99981 | 1 | 17693 | 0 | −1.4721 |
| MED20 | 4 | 0.00019591 | 0.00073683 | 0.029044 | 458 | 4 | −1.9158 | 0.99866 | 0.99868 | 1 | 17315 | 0 | −1.9158 |
| CARS2 | 4 | 0.00019645 | 0.00074067 | 0.029131 | 459 | 4 | −3.2625 | 0.9998 | 0.99981 | 1 | 17691 | 0 | −3.2625 |
| IPO11 | 4 | 0.00020251 | 0.00075932 | 0.0298 | 460 | 4 | −2.121 | 0.9998 | 0.99981 | 1 | 17689 | 0 | −2.121 |
| SCAP | 4 | 0.00020357 | 0.00076315 | 0.029886 | 461 | 4 | −3.4026 | 0.99967 | 0.99969 | 1 | 17610 | 0 | −3.4026 |
| FTSJ2 | 4 | 0.00020453 | 0.00076699 | 0.029971 | 462 | 4 | −2.1987 | 0.9998 | 0.99981 | 1 | 17687 | 0 | −2.1987 |
| NHP2 | 4 | 0.00020696 | 0.00077248 | 0.03012 | 463 | 4 | −3.0425 | 0.99979 | 0.9998 | 1 | 17685 | 0 | −3.0425 |
| PSMB6 | 4 | 0.00020842 | 0.00077577 | 0.030183 | 464 | 4 | −1.5476 | 0.99979 | 0.9998 | 1 | 17684 | 0 | −1.5476 |
| TTC27 | 4 | 0.00021039 | 0.00078509 | 0.030434 | 465 | 4 | −1.5423 | 0.99979 | 0.9998 | 1 | 17683 | 0 | −1.5423 |
| SHFM1 | 4 | 0.00021078 | 0.00078564 | 0.030434 | 466 | 4 | −2.4818 | 0.99979 | 0.9998 | 1 | 17682 | 0 | −2.4818 |
| RFC4 | 4 | 0.00021138 | 0.00078729 | 0.030434 | 467 | 4 | −1.3278 | 0.99979 | 0.9998 | 1 | 17681 | 0 | −1.3278 |
| TAPT1 | 4 | 0.00021476 | 0.00079825 | 0.030748 | 468 | 4 | −1.3927 | 0.99979 | 0.99979 | 1 | 17680 | 0 | −1.3927 |
| NAA10 | 4 | 0.00021526 | 0.0007988 | 0.030748 | 469 | 4 | −3.3097 | 0.99978 | 0.99979 | 1 | 17679 | 0 | −3.3097 |
| POLD2 | 4 | 0.00021859 | 0.00081197 | 0.031158 | 470 | 4 | −3.9807 | 0.99978 | 0.99979 | 1 | 17678 | 0 | −3.9807 |
| MOCS3 | 4 | 0.00022012 | 0.0008158 | 0.031158 | 471 | 4 | −1.4307 | 0.99978 | 0.99979 | 1 | 17676 | 0 | −1.4307 |
| TBCA | 4 | 0.00022044 | 0.00081635 | 0.031158 | 472 | 4 | −3.8245 | 0.96385 | 0.96377 | 1 | 15980 | 0 | −3.8245 |
| EIF2B5 | 4 | 0.00022053 | 0.00081635 | 0.031158 | 473 | 4 | −1.4129 | 0.99978 | 0.99978 | 1 | 17675 | 0 | −1.4129 |
| POLR3A | 4 | 0.00022124 | 0.0008191 | 0.031196 | 474 | 4 | −3.0467 | 0.99978 | 0.99978 | 1 | 17674 | 0 | −3.0467 |
| LIMS1 | 4 | 0.00022361 | 0.00082952 | 0.03145 | 475 | 4 | −1.578 | 0.99978 | 0.99978 | 1 | 17672 | 0 | −1.578 |
| DYNLRB1 | 4 | 0.00022381 | 0.00083006 | 0.03145 | 476 | 4 | −2.8139 | 0.99978 | 0.99978 | 1 | 17671 | 0 | −2.8139 |
| HYPK | 4 | 0.00022444 | 0.00083226 | 0.03145 | 477 | 4 | −1.3634 | 0.99978 | 0.99978 | 1 | 17670 | 0 | −1.3634 |
| WDR5 | 4 | 0.00022506 | 0.00083335 | 0.03145 | 478 | 4 | −1.7899 | 0.99977 | 0.99978 | 1 | 17669 | 0 | −1.7899 |
| TFAM | 4 | 0.00022526 | 0.00083445 | 0.03145 | 479 | 4 | −2.5393 | 0.99977 | 0.99978 | 1 | 17668 | 0 | −2.5393 |
| EIF2S3 | 4 | 0.0002263 | 0.00084048 | 0.031611 | 480 | 4 | −1.9265 | 0.99977 | 0.99978 | 1 | 17667 | 0 | −1.9265 |
| RPS11 | 4 | 0.00022735 | 0.00084487 | 0.03171 | 481 | 4 | −3.048 | 0.99977 | 0.99978 | 1 | 17666 | 0 | −3.048 |
| DARS | 4 | 0.00022829 | 0.00084981 | 0.031829 | 482 | 4 | −2.9591 | 0.99977 | 0.99978 | 1 | 17665 | 0 | −2.9591 |
| UBE2C | 4 | 0.00022997 | 0.00085694 | 0.03203 | 483 | 4 | −1.9417 | 0.99977 | 0.99978 | 1 | 17664 | 0 | −1.9417 |
| RPL11 | 4 | 0.00023092 | 0.00085968 | 0.032066 | 484 | 4 | −2.8111 | 0.99977 | 0.99977 | 1 | 17663 | 0 | −2.8111 |
| NDC1 | 4 | 0.00023209 | 0.00086407 | 0.032092 | 485 | 4 | −1.188 | 0.99977 | 0.99977 | 1 | 17662 | 0 | −1.188 |
| DTYMK | 4 | 0.00023244 | 0.00086516 | 0.032092 | 486 | 4 | −3.4657 | 0.98934 | 0.98946 | 1 | 16664 | 0 | −3.4657 |
| KRR1 | 4 | 0.00023294 | 0.00086571 | 0.032092 | 487 | 4 | −2.0529 | 0.97317 | 0.97312 | 1 | 16191 | 0 | −2.0529 |
| ARID4A | 4 | 0.00023358 | 0.00086845 | 0.032118 | 488 | 4 | −1.4958 | 0.99977 | 0.99977 | 1 | 17661 | 0 | −1.4958 |
| RIOK2 | 4 | 0.00023443 | 0.0008712 | 0.032118 | 489 | 4 | −1.8235 | 0.99977 | 0.99977 | 1 | 17660 | 0 | −1.8235 |
| TUBGCP3 | 4 | 0.00023469 | 0.00087175 | 0.032118 | 490 | 4 | −2.0063 | 0.99938 | 0.9994 | 1 | 17480 | 0 | −2.0063 |
| LIAS | 4 | 0.00023594 | 0.00087613 | 0.032214 | 491 | 4 | −3.1581 | 0.99393 | 0.99403 | 1 | 16895 | 0 | −3.1581 |
| RAD51D | 4 | 0.00023809 | 0.00088162 | 0.032349 | 492 | 4 | −1.8308 | 0.99976 | 0.99977 | 1 | 17658 | 0 | −1.8308 |
| MBTPS2 | 4 | 0.00023897 | 0.00088546 | 0.032413 | 493 | 3 | −1.3355 | 0.62824 | 0.75231 | 1 | 12401 | 1 | −1.3355 |
| B3GNT2 | 4 | 0.00023923 | 0.0008871 | 0.032413 | 494 | 3 | −1.2858 | 0.8691 | 0.8734 | 1 | 14330 | 1 | −1.2858 |
| PMPCA | 4 | 0.00024026 | 0.00088875 | 0.032413 | 495 | 4 | −2.1608 | 0.99976 | 0.99976 | 1 | 17657 | 0 | −2.1608 |
| PIK3CA | 4 | 0.00024366 | 0.00089588 | 0.032582 | 496 | 4 | −1.7322 | 0.99976 | 0.99976 | 1 | 17656 | 0 | −1.7322 |
| KLF3 | 4 | 0.00024388 | 0.00089697 | 0.032582 | 497 | 4 | −1.6357 | 0.99976 | 0.99976 | 1 | 17655 | 0 | −1.6357 |
| PSMG2 | 4 | 0.00024482 | 0.00090026 | 0.032635 | 498 | 4 | −1.5167 | 0.98959 | 0.98969 | 1 | 16674 | 0 | −1.5167 |
| IMPDH2 | 4 | 0.00024744 | 0.00091014 | 0.032816 | 499 | 4 | −2.6658 | 0.99762 | 0.99764 | 1 | 17182 | 0 | −2.6658 |
| CHCHD4 | 4 | 0.00024754 | 0.00091068 | 0.032816 | 500 | 4 | −1.7153 | 0.99975 | 0.99976 | 1 | 17654 | 0 | −1.7153 |
| POLA1 | 4 | 0.00024798 | 0.00091068 | 0.032816 | 501 | 4 | −1.5439 | 0.99975 | 0.99976 | 1 | 17653 | 0 | −1.5439 |
| C1QBP | 4 | 0.00025057 | 0.00091836 | 0.033013 | 502 | 4 | −2.2673 | 0.99956 | 0.99958 | 1 | 17542 | 0 | −2.2673 |
| SAP18 | 4 | 0.00025112 | 0.00092111 | 0.033013 | 503 | 4 | −2.9135 | 0.99975 | 0.99975 | 1 | 17652 | 0 | −2.9135 |
| ATP5A1 | 4 | 0.00025123 | 0.00092165 | 0.033013 | 504 | 4 | −1.4054 | 0.99975 | 0.99975 | 1 | 17651 | 0 | −1.4054 |
| HNRNPU | 4 | 0.00025168 | 0.00092385 | 0.033026 | 505 | 4 | −3.4343 | 0.99975 | 0.99975 | 1 | 17650 | 0 | −3.4343 |
| RAB10 | 4 | 0.00025315 | 0.00092769 | 0.033098 | 506 | 4 | −1.7563 | 0.99975 | 0.99975 | 1 | 17649 | 0 | −1.7563 |
| SMG5 | 4 | 0.00025504 | 0.00093482 | 0.033286 | 507 | 3 | −2.3988 | 0.83639 | 0.85001 | 1 | 13947 | 1 | −2.3988 |
| UQCR10 | 4 | 0.00025567 | 0.00093756 | 0.033318 | 508 | 3 | −0.92823 | 0.63285 | 0.75391 | 1 | 12423 | 1 | −0.92823 |
| ADNP | 4 | 0.00025676 | 0.0009403 | 0.03335 | 509 | 4 | −1.6082 | 0.9897 | 0.9898 | 1 | 16678 | 0 | −1.6082 |
| FXN | 4 | 0.00026065 | 0.00095621 | 0.033848 | 510 | 4 | −2.3761 | 0.99958 | 0.9996 | 1 | 17553 | 0 | −2.3761 |
| DDX55 | 4 | 0.00026199 | 0.00096224 | 0.033995 | 511 | 4 | −2.5327 | 0.98115 | 0.98122 | 1 | 16384 | 0 | −2.5327 |
| SSR3 | 4 | 0.00026491 | 0.00097485 | 0.034373 | 512 | 4 | −1.6785 | 0.99974 | 0.99974 | 1 | 17646 | 0 | −1.6785 |
| ARPC4 | 4 | 0.00026632 | 0.00097869 | 0.034441 | 513 | 4 | −2.2261 | 0.99973 | 0.99974 | 1 | 17645 | 0 | −2.2261 |
| SETD2 | 4 | 0.00026742 | 0.00098143 | 0.03447 | 514 | 4 | −2.8422 | 0.99947 | 0.99949 | 1 | 17511 | 0 | −2.8422 |
| RNASEH2C | 4 | 0.00026963 | 0.00098801 | 0.034634 | 515 | 4 | −0.8906 | 0.99973 | 0.99974 | 1 | 17644 | 0 | −0.8906 |
| NDUFB10 | 4 | 0.00027011 | 0.00099021 | 0.034644 | 516 | 4 | −1.9548 | 0.99973 | 0.99973 | 1 | 17643 | 0 | −1.9548 |
| UTP14A | 4 | 0.00027098 | 0.0009935 | 0.034692 | 517 | 4 | −2.1783 | 0.98991 | 0.99 | 1 | 16690 | 0 | −2.1783 |
| PSMD13 | 4 | 0.00027225 | 0.0010012 | 0.034815 | 518 | 4 | −2.5212 | 0.99973 | 0.99973 | 1 | 17642 | 0 | −2.5212 |
| CFL1 | 4 | 0.00027285 | 0.0010023 | 0.034815 | 519 | 4 | −1.7982 | 0.99973 | 0.99973 | 1 | 17641 | 0 | −1.7982 |
| NDUFA13 | 4 | 0.00027292 | 0.0010028 | 0.034815 | 520 | 4 | −4.0132 | 0.99235 | 0.99247 | 1 | 16814 | 0 | −4.0132 |
| TUBGCP4 | 4 | 0.00027538 | 0.0010094 | 0.034938 | 521 | 4 | −2.5743 | 0.99972 | 0.99973 | 1 | 17640 | 0 | −2.5743 |
| FOXRED1 | 4 | 0.0002755 | 0.0010102 | 0.034938 | 522 | 4 | −1.4451 | 0.99972 | 0.99973 | 1 | 17639 | 0 | −1.4451 |
| METTL3 | 4 | 0.00027691 | 0.0010132 | 0.034975 | 523 | 4 | −2.4296 | 0.99903 | 0.99901 | 1 | 17393 | 0 | −2.4296 |
| TTI2 | 4 | 0.00027934 | 0.0010204 | 0.035019 | 524 | 4 | −1.7242 | 0.97533 | 0.9753 | 1 | 16244 | 0 | −1.7242 |
| ZNF410 | 4 | 0.00027962 | 0.001022 | 0.035019 | 525 | 4 | −1.3431 | 0.99871 | 0.99871 | 1 | 17327 | 0 | −1.3431 |
| GPN2 | 4 | 0.00028024 | 0.0010231 | 0.035019 | 526 | 4 | −3.1184 | 0.99972 | 0.99973 | 1 | 17637 | 0 | −3.1184 |
| ARF1 | 4 | 0.00028073 | 0.0010242 | 0.035019 | 527 | 4 | −1.2984 | 0.99972 | 0.99973 | 1 | 17636 | 0 | −1.2984 |
| PDCD11 | 4 | 0.00028074 | 0.0010242 | 0.035019 | 528 | 4 | −3.0387 | 0.9947 | 0.9948 | 1 | 16952 | 0 | −3.0387 |
| NDUFA9 | 4 | 0.00028272 | 0.0010352 | 0.035327 | 529 | 4 | −1.8957 | 0.98334 | 0.98342 | 1 | 16458 | 0 | −1.8957 |
| DCTN2 | 4 | 0.00028306 | 0.0010379 | 0.035354 | 530 | 4 | −2.3445 | 0.99972 | 0.99973 | 1 | 17635 | 0 | −2.3445 |
| RANBP3 | 4 | 0.00028471 | 0.0010461 | 0.035456 | 531 | 3 | −1.4741 | 0.56844 | 0.71225 | 1 | 11772 | 1 | −1.4741 |
| PARP1 | 4 | 0.00028516 | 0.0010467 | 0.035456 | 532 | 4 | −1.1889 | 0.99971 | 0.99972 | 1 | 17634 | 0 | −1.1889 |
| PPP1R8 | 4 | 0.00028585 | 0.0010483 | 0.035456 | 533 | 4 | −2.1303 | 0.99958 | 0.9996 | 1 | 17558 | 0 | −2.1303 |
| SRP9 | 4 | 0.00028614 | 0.0010494 | 0.035456 | 534 | 3 | −1.8521 | 0.80357 | 0.8301 | 1 | 13603 | 1 | −1.8521 |
| RQCD1 | 4 | 0.00028715 | 0.0010533 | 0.035456 | 535 | 4 | −1.8275 | 0.99971 | 0.99972 | 1 | 17633 | 0 | −1.8275 |
| GALE | 4 | 0.0002894 | 0.0010593 | 0.035456 | 536 | 4 | −1.5357 | 0.99971 | 0.99972 | 1 | 17631 | 0 | −1.5357 |
| NCAPH | 4 | 0.00028958 | 0.0010593 | 0.035456 | 537 | 4 | −1.9104 | 0.98986 | 0.98994 | 1 | 16686 | 0 | −1.9104 |
| SMARCD2 | 4 | 0.0002899 | 0.0010599 | 0.035456 | 538 | 4 | −1.7078 | 0.99971 | 0.99972 | 1 | 17630 | 0 | −1.7078 |
| ANAPC2 | 4 | 0.00029028 | 0.001061 | 0.035456 | 539 | 4 | −2.7143 | 0.99971 | 0.99972 | 1 | 17629 | 0 | −2.7143 |
| MRPS11 | 4 | 0.00029078 | 0.0010637 | 0.035456 | 540 | 4 | −1.4298 | 0.99971 | 0.99972 | 1 | 17628 | 0 | −1.4298 |
| MTHFD1 | 4 | 0.00029103 | 0.0010648 | 0.035456 | 541 | 4 | −2.5507 | 0.99971 | 0.99972 | 1 | 17627 | 0 | −2.5507 |
| MED28 | 4 | 0.00029153 | 0.0010664 | 0.035456 | 542 | 4 | −1.8609 | 0.99971 | 0.99972 | 1 | 17626 | 0 | −1.8609 |
| POLR1C | 4 | 0.0002916 | 0.0010664 | 0.035456 | 543 | 4 | −2.1031 | 0.99176 | 0.99188 | 1 | 16774 | 0 | −2.1031 |
| VPS8 | 4 | 0.00029218 | 0.0010719 | 0.035544 | 544 | 4 | −1.803 | 0.99898 | 0.99897 | 1 | 17379 | 0 | −1.803 |
| RUVBL1 | 4 | 0.00029254 | 0.001073 | 0.035544 | 545 | 4 | −2.8211 | 0.99971 | 0.99972 | 1 | 17625 | 0 | −2.8211 |
| LARS2 | 4 | 0.00029406 | 0.0010769 | 0.035605 | 546 | 4 | −1.6643 | 0.99971 | 0.99972 | 1 | 17624 | 0 | −1.6643 |
| TMEM258 | 4 | 0.00029559 | 0.0010823 | 0.035721 | 547 | 4 | −1.3798 | 0.9997 | 0.99972 | 1 | 17623 | 0 | −1.3798 |
| SAMM50 | 4 | 0.00029889 | 0.0010917 | 0.035941 | 548 | 4 | −2.632 | 0.99723 | 0.99726 | 1 | 17137 | 0 | −2.632 |
| ECSIT | 4 | 0.00030018 | 0.0010944 | 0.035941 | 549 | 3 | −1.6811 | 0.75193 | 0.80311 | 1 | 13165 | 1 | −1.6811 |
| SDHA | 4 | 0.00030036 | 0.001095 | 0.035941 | 550 | 4 | −3.2368 | 0.99957 | 0.99959 | 1 | 17551 | 0 | −3.2368 |
| EIF3H | 4 | 0.00030369 | 0.0011065 | 0.036188 | 551 | 4 | −1.3019 | 0.9997 | 0.99971 | 1 | 17622 | 0 | −1.3019 |
| ATP6V1B2 | 4 | 0.00030434 | 0.0011087 | 0.036188 | 552 | 4 | −1.6657 | 0.9997 | 0.99971 | 1 | 17621 | 0 | −1.6657 |
| DHPS | 4 | 0.00030451 | 0.0011092 | 0.036188 | 553 | 4 | −5.3847 | 0.99768 | 0.99771 | 1 | 17192 | 0 | −5.3847 |
| NUDC | 4 | 0.0003057 | 0.001112 | 0.036188 | 554 | 3 | −2.3846 | 0.92179 | 0.92159 | 1 | 15155 | 0 | −2.3846 |
| FECH | 4 | 0.00030655 | 0.0011125 | 0.036188 | 555 | 4 | −1.7718 | 0.99969 | 0.99971 | 1 | 17620 | 0 | −1.7718 |
| CCT2 | 4 | 0.0003076 | 0.0011174 | 0.036283 | 556 | 4 | −2.822 | 0.99969 | 0.99971 | 1 | 17619 | 0 | −2.822 |
| DNM2 | 4 | 0.00030957 | 0.0011224 | 0.036313 | 557 | 4 | −2.1916 | 0.99969 | 0.99971 | 1 | 17618 | 0 | −2.1916 |
| ALAD | 4 | 0.00030975 | 0.0011224 | 0.036313 | 558 | 3 | −2.5629 | 0.51986 | 0.67042 | 1 | 11126 | 1 | −2.5629 |
| STRA13 | 4 | 0.00031023 | 0.0011262 | 0.036372 | 559 | 4 | −1.2393 | 0.99969 | 0.9997 | 1 | 17617 | 0 | −1.2393 |
| HTATSF1 | 4 | 0.00031142 | 0.0011301 | 0.03643 | 560 | 4 | −2.1241 | 0.99969 | 0.9997 | 1 | 17616 | 0 | −2.1241 |
| TAF1B | 4 | 0.00031322 | 0.0011372 | 0.036595 | 561 | 3 | −1.477 | 0.92872 | 0.92847 | 1 | 15281 | 0 | −1.477 |
| TGFBRAP1 | 4 | 0.00031408 | 0.0011399 | 0.036618 | 562 | 4 | −1.5963 | 0.99969 | 0.9997 | 1 | 17615 | 0 | −1.5963 |
| PPP1R12A | 4 | 0.00031568 | 0.0011476 | 0.036799 | 563 | 4 | −1.5526 | 0.99968 | 0.9997 | 1 | 17614 | 0 | −1.5526 |
| MTCH2 | 4 | 0.00031809 | 0.0011542 | 0.036944 | 564 | 4 | −1.2204 | 0.99968 | 0.9997 | 1 | 17613 | 0 | −1.2204 |
| MRPS18B | 4 | 0.0003184 | 0.0011575 | 0.036984 | 565 | 4 | −2.2593 | 0.99945 | 0.99947 | 1 | 17506 | 0 | −2.2593 |
| MCTS1 | 4 | 0.00032179 | 0.0011674 | 0.037234 | 566 | 4 | −2.4377 | 0.99902 | 0.999 | 1 | 17391 | 0 | −2.4377 |
| C1GALT1 | 4 | 0.00032351 | 0.0011701 | 0.037255 | 567 | 4 | −1.1631 | 0.99968 | 0.99969 | 1 | 17611 | 0 | −1.1631 |
| POLR1E | 4 | 0.00032761 | 0.0011838 | 0.037626 | 568 | 4 | −1.6834 | 0.99967 | 0.99969 | 1 | 17609 | 0 | −1.6834 |
| BRPF1 | 4 | 0.00032996 | 0.0011926 | 0.037838 | 569 | 4 | −1.9318 | 0.99967 | 0.99969 | 1 | 17608 | 0 | −1.9318 |
| WDR48 | 4 | 0.00033461 | 0.0012118 | 0.038379 | 570 | 3 | −2.7394 | 0.51787 | 0.66871 | 1 | 11103 | 1 | −2.7394 |
| WDR83 | 4 | 0.00033664 | 0.0012167 | 0.038453 | 571 | 4 | −1.1523 | 0.99966 | 0.99968 | 1 | 17606 | 0 | −1.1523 |
| ASNA1 | 4 | 0.00033748 | 0.0012184 | 0.038453 | 572 | 4 | −3.0798 | 0.99966 | 0.99968 | 1 | 17605 | 0 | −3.0798 |
| DNAJB11 | 4 | 0.00033875 | 0.0012244 | 0.038543 | 573 | 4 | −1.5105 | 0.99966 | 0.99968 | 1 | 17604 | 0 | −1.5105 |
| ORC6 | 4 | 0.00033906 | 0.0012255 | 0.038543 | 574 | 4 | −1.713 | 0.99957 | 0.99959 | 1 | 17547 | 0 | −1.713 |
| TBP | 4 | 0.00034034 | 0.0012293 | 0.038581 | 575 | 3 | −3.2389 | 0.67904 | 0.7712 | 1 | 12681 | 1 | −3.2389 |
| FEN1 | 4 | 0.00034162 | 0.001231 | 0.038581 | 576 | 3 | −2.1926 | 0.77933 | 0.8168 | 1 | 13375 | 1 | −2.1926 |
| TPT1 | 4 | 0.00034257 | 0.0012359 | 0.038669 | 577 | 4 | −2.9673 | 0.99966 | 0.99968 | 1 | 17603 | 0 | −2.9673 |
| MED16 | 4 | 0.00034291 | 0.0012381 | 0.03867 | 578 | 4 | −1.6004 | 0.99929 | 0.9993 | 1 | 17451 | 0 | −1.6004 |
| NUDT21 | 4 | 0.00034442 | 0.0012447 | 0.038709 | 579 | 4 | −3.2499 | 0.99966 | 0.99967 | 1 | 17602 | 0 | −3.2499 |
| WDR55 | 4 | 0.00034484 | 0.0012452 | 0.038709 | 580 | 3 | −2.3357 | 0.56934 | 0.71303 | 1 | 11787 | 1 | −2.3357 |
| CNOT1 | 4 | 0.00034514 | 0.0012458 | 0.038709 | 581 | 4 | −2.2145 | 0.99965 | 0.99967 | 1 | 17601 | 0 | −2.2145 |
| WDHD1 | 4 | 0.00034643 | 0.0012502 | 0.038737 | 582 | 4 | −2.1211 | 0.99965 | 0.99967 | 1 | 17600 | 0 | −2.1211 |
| MAK16 | 4 | 0.000347 | 0.0012524 | 0.038737 | 583 | 4 | −1.9999 | 0.99965 | 0.99967 | 1 | 17599 | 0 | −1.9999 |
| PNKP | 4 | 0.00034786 | 0.0012573 | 0.038737 | 584 | 4 | −1.4859 | 0.99965 | 0.99967 | 1 | 17597 | 0 | −1.4859 |
| HSPA8 | 4 | 0.00034823 | 0.0012589 | 0.038737 | 585 | 3 | −3.7221 | 0.86381 | 0.86934 | 1 | 14269 | 1 | −3.7221 |
| VCP | 4 | 0.00034858 | 0.00126 | 0.038737 | 586 | 4 | −2.1271 | 0.99965 | 0.99967 | 1 | 17596 | 0 | −2.1271 |
| NDUFA11 | 4 | 0.00034873 | 0.0012606 | 0.038737 | 587 | 4 | −2.5478 | 0.99415 | 0.99427 | 1 | 16917 | 0 | −2.5478 |
| PDE12 | 4 | 0.00034945 | 0.0012617 | 0.038737 | 588 | 4 | −1.352 | 0.99965 | 0.99967 | 1 | 17595 | 0 | −1.352 |
| IDI1 | 4 | 0.00035207 | 0.0012694 | 0.038906 | 589 | 2 | −0.90454 | 0.38711 | 0.55853 | 1 | 9512 | 2 | −0.90454 |
| DGKQ | 4 | 0.00035336 | 0.0012721 | 0.038924 | 590 | 4 | −1.4522 | 0.99965 | 0.99966 | 1 | 17594 | 0 | −1.4522 |
| ARMC5 | 4 | 0.00035594 | 0.0012858 | 0.039277 | 591 | 3 | −1.8342 | 0.44548 | 0.60759 | 1 | 10216 | 1 | −1.8342 |
| TUBE1 | 4 | 0.00035687 | 0.0012919 | 0.039395 | 592 | 4 | −2.9681 | 0.99964 | 0.99966 | 1 | 17593 | 0 | −2.9681 |
| ATP5B | 4 | 0.00035877 | 0.0012957 | 0.039445 | 593 | 4 | −1.6985 | 0.99964 | 0.99965 | 1 | 17592 | 0 | −1.6985 |
| KIDINS220 | 4 | 0.00036084 | 0.0013017 | 0.039562 | 594 | 4 | −1.9228 | 0.99964 | 0.99965 | 1 | 17591 | 0 | −1.9228 |
| MCAT | 4 | 0.00036124 | 0.0013039 | 0.039562 | 595 | 3 | −2.2004 | 0.84142 | 0.85332 | 1 | 14010 | 1 | −2.2004 |
| PPP2R1A | 4 | 0.00036291 | 0.0013143 | 0.039812 | 596 | 4 | −2.1372 | 0.99964 | 0.99965 | 1 | 17590 | 0 | −2.1372 |
| DHX29 | 4 | 0.0003644 | 0.001322 | 0.039977 | 597 | 4 | −1.0876 | 0.99964 | 0.99965 | 1 | 17589 | 0 | −1.0876 |
| TMED2 | 4 | 0.00036485 | 0.0013264 | 0.040043 | 598 | 1 | −1.6779 | 0.99964 | 0.99965 | 1 | 17588 | 0 | −1.6779 |
| LUC7L3 | 4 | 0.00036828 | 0.0013418 | 0.040295 | 599 | 3 | −2.7992 | 0.88204 | 0.88369 | 1 | 14525 | 1 | −2.7992 |
| EEF1D | 4 | 0.00036896 | 0.001344 | 0.040295 | 600 | 4 | −1.303 | 0.99214 | 0.99226 | 1 | 16795 | 0 | −1.303 |
| ATXN10 | 4 | 0.0003693 | 0.0013445 | 0.040295 | 601 | 3 | −1.8474 | 0.95135 | 0.95125 | 1 | 15728 | 0 | −1.8474 |
| GNL3L | 4 | 0.00036979 | 0.0013461 | 0.040295 | 602 | 4 | −1.2555 | 0.99963 | 0.99964 | 1 | 17587 | 0 | −1.2555 |
| SRP14 | 4 | 0.00037031 | 0.0013467 | 0.040295 | 603 | 4 | −1.6772 | 0.99899 | 0.99897 | 1 | 17381 | 0 | −1.6772 |
| NOL10 | 4 | 0.0003713 | 0.0013494 | 0.040295 | 604 | 4 | −3.2685 | 0.99963 | 0.99964 | 1 | 17586 | 0 | −3.2685 |
| EIF3A | 4 | 0.00037235 | 0.0013538 | 0.040295 | 605 | 4 | −2.6711 | 0.99837 | 0.99839 | 1 | 17268 | 0 | −2.6711 |
| GEMIN5 | 4 | 0.00037387 | 0.0013571 | 0.040295 | 606 | 4 | −2.2473 | 0.99963 | 0.99964 | 1 | 17584 | 0 | −2.2473 |
| CHORDC1 | 4 | 0.00037439 | 0.0013577 | 0.040295 | 607 | 4 | −2.2221 | 0.99704 | 0.99707 | 1 | 17118 | 0 | −2.2221 |
| HYOU1 | 4 | 0.00037478 | 0.0013582 | 0.040295 | 608 | 4 | −2.4333 | 0.99963 | 0.99964 | 1 | 17583 | 0 | −2.4333 |
| RNPS1 | 4 | 0.00037494 | 0.0013593 | 0.040295 | 609 | 4 | −1.8833 | 0.99963 | 0.99964 | 1 | 17582 | 0 | −1.8833 |
| PES1 | 4 | 0.00037738 | 0.0013664 | 0.04044 | 610 | 4 | −2.7397 | 0.99962 | 0.99964 | 1 | 17581 | 0 | −2.7397 |
| NCBP1 | 4 | 0.00038029 | 0.0013801 | 0.040689 | 611 | 4 | −1.6775 | 0.99962 | 0.99963 | 1 | 17580 | 0 | −1.6775 |
| JAK1 | 4 | 0.00038125 | 0.0013834 | 0.040689 | 612 | 4 | −2.2548 | 0.9888 | 0.98893 | 1 | 16641 | 0 | −2.2548 |
| PDCL3 | 4 | 0.00038183 | 0.001384 | 0.040689 | 613 | 4 | −1.4549 | 0.99962 | 0.99963 | 1 | 17579 | 0 | −1.4549 |
| ADSL | 4 | 0.00038337 | 0.0013867 | 0.040689 | 614 | 4 | −2.8972 | 0.99962 | 0.99963 | 1 | 17578 | 0 | −2.8972 |
| SDAD1 | 4 | 0.00038367 | 0.0013873 | 0.040689 | 615 | 3 | −2.1768 | 0.7494 | 0.80184 | 1 | 13152 | 1 | −2.1768 |
| DERL1 | 4 | 0.00038415 | 0.0013884 | 0.040689 | 616 | 4 | −1.8013 | 0.99962 | 0.99963 | 1 | 17577 | 0 | −1.8013 |
| RNF20 | 4 | 0.00038617 | 0.0013966 | 0.040798 | 617 | 4 | −1.5683 | 0.99961 | 0.99963 | 1 | 17576 | 0 | −1.5683 |
| CTCF | 4 | 0.00038632 | 0.0013966 | 0.040798 | 618 | 4 | −1.7999 | 0.99961 | 0.99963 | 1 | 17575 | 0 | −1.7999 |
| FTSJ3 | 4 | 0.00038772 | 0.001401 | 0.04086 | 619 | 4 | −3.0816 | 0.99961 | 0.99962 | 1 | 17574 | 0 | −3.0816 |
| NDUFB11 | 4 | 0.00039117 | 0.0014158 | 0.041225 | 620 | 4 | −1.8491 | 0.99961 | 0.99962 | 1 | 17573 | 0 | −1.8491 |
| NRAS | 4 | 0.00039542 | 0.0014306 | 0.041535 | 621 | 4 | −0.86387 | 0.9996 | 0.99962 | 1 | 17572 | 0 | −0.86387 |
| RPRD1B | 4 | 0.00039557 | 0.0014312 | 0.041535 | 622 | 3 | −1.946 | 0.78835 | 0.82162 | 1 | 13452 | 1 | −1.946 |
| UBTF | 4 | 0.00039653 | 0.0014333 | 0.041535 | 623 | 4 | −1.7287 | 0.9996 | 0.99962 | 1 | 17571 | 0 | −1.7287 |
| DLD | 4 | 0.00039947 | 0.0014421 | 0.041557 | 624 | 4 | −1.9258 | 0.9996 | 0.99961 | 1 | 17565 | 0 | −1.9258 |
| ZNF407 | 4 | 0.00040019 | 0.0014454 | 0.041557 | 625 | 4 | −2.6364 | 0.99951 | 0.99953 | 1 | 17524 | 0 | −2.6364 |
| ALG3 | 4 | 0.0004002 | 0.0014454 | 0.041557 | 626 | 4 | −1.6919 | 0.9996 | 0.99962 | 1 | 17570 | 0 | −1.6919 |
| THOC6 | 4 | 0.00040147 | 0.0014482 | 0.041557 | 627 | 4 | −2.3462 | 0.9996 | 0.99962 | 1 | 17569 | 0 | −2.3462 |
| STRAP | 4 | 0.00040179 | 0.0014493 | 0.041557 | 628 | 4 | −2.6613 | 0.9996 | 0.99962 | 1 | 17568 | 0 | −2.6613 |
| BPTF | 4 | 0.00040212 | 0.0014509 | 0.041557 | 629 | 4 | −1.7414 | 0.9996 | 0.99962 | 1 | 17567 | 0 | −1.7414 |
| HNRNPUL1 | 4 | 0.00040228 | 0.001452 | 0.041557 | 630 | 4 | −1.5764 | 0.9996 | 0.99962 | 1 | 17566 | 0 | −1.5764 |
| FNTB | 4 | 0.00040269 | 0.0014525 | 0.041557 | 631 | 4 | −1.8896 | 0.98831 | 0.9884 | 1 | 16628 | 0 | −1.8896 |
| EXOSC1 | 4 | 0.0004042 | 0.0014575 | 0.041592 | 632 | 4 | −1.2227 | 0.9996 | 0.99961 | 1 | 17564 | 0 | −1.2227 |
| CNOT4 | 4 | 0.00040453 | 0.0014599 | 0.041592 | 633 | 4 | −1.3664 | 0.9996 | 0.99961 | 1 | 17563 | 0 | −1.3664 |
| C17orf70 | 4 | 0.00040501 | 0.0014619 | 0.041592 | 634 | 4 | −1.3852 | 0.99959 | 0.99961 | 1 | 17562 | 0 | −1.3852 |
| EIF2AK4 | 4 | 0.0004052 | 0.001463 | 0.041592 | 635 | 3 | −3.408 | 0.82156 | 0.84068 | 1 | 13778 | 1 | −3.408 |
| PUF60 | 4 | 0.00040727 | 0.0014745 | 0.041834 | 636 | 4 | −2.3697 | 0.99959 | 0.99961 | 1 | 17561 | 0 | −2.3697 |
| MDM4 | 4 | 0.00040805 | 0.0014761 | 0.041834 | 637 | 4 | −2.2963 | 0.99481 | 0.99491 | 1 | 16955 | 0 | −2.2963 |
| RFC2 | 4 | 0.0004097 | 0.0014811 | 0.041909 | 638 | 4 | −1.7513 | 0.99959 | 0.99961 | 1 | 17559 | 0 | −1.7513 |
| C17orf89 | 4 | 0.00041625 | 0.0015046 | 0.042443 | 639 | 4 | −1.2429 | 0.99958 | 0.9996 | 1 | 17557 | 0 | −1.2429 |
| SUZ12 | 4 | 0.00041643 | 0.0015046 | 0.042443 | 640 | 3 | −2.2776 | 0.47499 | 0.63256 | 1 | 10566 | 1 | −2.2776 |
| SUPT4H1 | 4 | 0.00041826 | 0.001509 | 0.0425 | 641 | 3 | −4.1108 | 0.8391 | 0.85177 | 1 | 13977 | 1 | −4.1108 |
| DDA1 | 4 | 0.00042037 | 0.0015173 | 0.042599 | 642 | 4 | −0.97867 | 0.99958 | 0.9996 | 1 | 17556 | 0 | −0.97867 |
| MRPS24 | 4 | 0.00042047 | 0.0015173 | 0.042599 | 643 | 4 | −1.9627 | 0.99881 | 0.9988 | 1 | 17351 | 0 | −1.9627 |
| RPP38 | 4 | 0.0004217 | 0.0015227 | 0.042636 | 644 | 4 | −1.6953 | 0.99958 | 0.9996 | 1 | 17555 | 0 | −1.6953 |
| NCL | 4 | 0.00042237 | 0.0015233 | 0.042636 | 645 | 4 | −2.5038 | 0.99958 | 0.9996 | 1 | 17554 | 0 | −2.5038 |
| PTBP1 | 4 | 0.00042654 | 0.0015332 | 0.042846 | 646 | 4 | −1.56 | 0.99957 | 0.99959 | 1 | 17552 | 0 | −1.56 |
| COPS6 | 4 | 0.00042906 | 0.0015419 | 0.043024 | 647 | 4 | −2.4681 | 0.99957 | 0.99959 | 1 | 17550 | 0 | −2.4681 |
| RBM10 | 4 | 0.00043176 | 0.0015496 | 0.04312 | 648 | 4 | −1.4782 | 0.99957 | 0.99959 | 1 | 17549 | 0 | −1.4782 |
| MRPL11 | 4 | 0.0004321 | 0.0015502 | 0.04312 | 649 | 4 | −1.9658 | 0.99957 | 0.99959 | 1 | 17548 | 0 | −1.9658 |
| CDK7 | 4 | 0.00043423 | 0.0015578 | 0.043195 | 650 | 4 | −3.2506 | 0.99627 | 0.99635 | 1 | 17058 | 0 | −3.2506 |
| NUBPL | 4 | 0.00043532 | 0.00156 | 0.043195 | 651 | 4 | −1.815 | 0.99956 | 0.99959 | 1 | 17546 | 0 | −1.815 |
| HRAS | 4 | 0.00043649 | 0.0015644 | 0.04325 | 652 | 4 | −1.5883 | 0.99861 | 0.99863 | 1 | 17305 | 0 | −1.5883 |
| IQGAP3 | 4 | 0.00043908 | 0.0015737 | 0.043442 | 653 | 4 | −1.3297 | 0.99956 | 0.99958 | 1 | 17545 | 0 | −1.3297 |
| TARS2 | 4 | 0.00044045 | 0.0015803 | 0.043557 | 654 | 4 | −1.9135 | 0.99956 | 0.99958 | 1 | 17544 | 0 | −1.9135 |
| HSP90B1 | 4 | 0.0004432 | 0.0015908 | 0.043741 | 655 | 4 | −1.7125 | 0.99956 | 0.99958 | 1 | 17543 | 0 | −1.7125 |
| SSR2 | 4 | 0.00044332 | 0.0015918 | 0.043741 | 656 | 4 | −2.0053 | 0.99253 | 0.99265 | 1 | 16825 | 0 | −2.0053 |
| BCCIP | 4 | 0.00044561 | 0.0016006 | 0.043878 | 657 | 3 | −1.754 | 0.83927 | 0.85189 | 1 | 13980 | 1 | −1.754 |
| TXN2 | 4 | 0.00044599 | 0.0016017 | 0.043878 | 658 | 3 | −3.2323 | 0.56133 | 0.70619 | 1 | 11684 | 1 | −3.2323 |
| TFRC | 4 | 0.0004477 | 0.0016105 | 0.044052 | 659 | 4 | −2.1117 | 0.99955 | 0.99957 | 1 | 17541 | 0 | −2.1117 |
| CTNNBL1 | 4 | 0.00045241 | 0.0016319 | 0.044517 | 660 | 4 | −2.1039 | 0.99955 | 0.99957 | 1 | 17540 | 0 | −2.1039 |
| TFB1M | 4 | 0.00045343 | 0.0016346 | 0.044517 | 661 | 3 | −2.3054 | 0.4804 | 0.6371 | 1 | 10631 | 1 | −2.3054 |
| CLNS1A | 4 | 0.00045364 | 0.0016352 | 0.044517 | 662 | 4 | −3.5663 | 0.99955 | 0.99957 | 1 | 17538 | 0 | −3.5663 |
| TIMM22 | 4 | 0.00045407 | 0.0016374 | 0.044517 | 663 | 4 | −1.9581 | 0.96356 | 0.96345 | 1 | 15973 | 0 | −1.9581 |
| UQCRFS1 | 4 | 0.00045505 | 0.0016407 | 0.04454 | 664 | 4 | −3.7186 | 0.99954 | 0.99957 | 1 | 17537 | 0 | −3.7186 |
| THAP1 | 4 | 0.00045757 | 0.0016494 | 0.044658 | 665 | 3 | −2.5217 | 0.92572 | 0.92549 | 1 | 15233 | 0 | −2.5217 |
| DHDDS | 4 | 0.00045787 | 0.00165 | 0.044658 | 666 | 4 | −2.3318 | 0.99954 | 0.99957 | 1 | 17536 | 0 | −2.3318 |
| PELO | 4 | 0.00045927 | 0.0016555 | 0.04474 | 667 | 4 | −1.8228 | 0.98315 | 0.98324 | 1 | 16451 | 0 | −1.8228 |
| GTF2H4 | 4 | 0.00046035 | 0.0016588 | 0.044762 | 668 | 4 | −1.2892 | 0.99954 | 0.99956 | 1 | 17535 | 0 | −1.2892 |
| RRP1 | 4 | 0.00046855 | 0.0016955 | 0.045578 | 669 | 4 | −1.9778 | 0.99129 | 0.9914 | 1 | 16753 | 0 | −1.9778 |
| PGK1 | 4 | 0.00046894 | 0.0016966 | 0.045578 | 670 | 3 | −4.0842 | 0.57111 | 0.71457 | 1 | 11805 | 1 | −4.0842 |
| ATP5F1 | 4 | 0.00047054 | 0.001701 | 0.045628 | 671 | 4 | −1.7296 | 0.99953 | 0.99955 | 1 | 17533 | 0 | −1.7296 |
| C14orf80 | 4 | 0.00047403 | 0.0017141 | 0.045759 | 672 | 2 | −1.5531 | 0.68875 | 0.77514 | 1 | 12745 | 1 | −1.5531 |
| TARDBP | 4 | 0.00047434 | 0.0017152 | 0.045759 | 673 | 4 | −3.8165 | 0.99953 | 0.99955 | 1 | 17532 | 0 | −3.8165 |
| TRMT112 | 4 | 0.00047489 | 0.0017174 | 0.045759 | 674 | 4 | −2.5443 | 0.99953 | 0.99955 | 1 | 17531 | 0 | −2.5443 |
| FBL | 4 | 0.0004758 | 0.0017185 | 0.045759 | 675 | 4 | −1.9219 | 0.99952 | 0.99955 | 1 | 17530 | 0 | −1.9219 |
| IGF1R | 4 | 0.00047707 | 0.0017235 | 0.045823 | 676 | 4 | −2.0168 | 0.99952 | 0.99954 | 1 | 17529 | 0 | −2.0168 |
| ELP6 | 4 | 0.00047982 | 0.0017339 | 0.046032 | 677 | 4 | −2.3969 | 0.9818 | 0.98189 | 1 | 16406 | 0 | −2.3969 |
| EXOC1 | 4 | 0.0004822 | 0.0017421 | 0.046183 | 678 | 4 | −1.723 | 0.99952 | 0.99954 | 1 | 17528 | 0 | −1.723 |
| G6PD | 4 | 0.00048615 | 0.0017531 | 0.046338 | 679 | 3 | −2.4066 | 0.89959 | 0.89948 | 1 | 14789 | 1 | −2.4066 |
| CHMP5 | 4 | 0.00048625 | 0.0017531 | 0.046338 | 680 | 4 | −1.372 | 0.99951 | 0.99954 | 1 | 17527 | 0 | −1.372 |
| CIRH1A | 4 | 0.00048752 | 0.0017558 | 0.046342 | 681 | 4 | −3.3376 | 0.99806 | 0.99809 | 1 | 17226 | 0 | −3.3376 |
| PSMB5 | 4 | 0.00049033 | 0.0017679 | 0.046525 | 682 | 4 | −1.8382 | 0.99951 | 0.99953 | 1 | 17526 | 0 | −1.8382 |
| ERBB3 | 4 | 0.00049103 | 0.0017712 | 0.046543 | 683 | 4 | −1.5641 | 0.98712 | 0.98721 | 1 | 16582 | 0 | −1.5641 |
| ACTR3 | 4 | 0.00049266 | 0.0017783 | 0.046663 | 684 | 3 | −2.3131 | 0.9452 | 0.94503 | 1 | 15598 | 0 | −2.3131 |
| HJURP | 4 | 0.00049406 | 0.0017833 | 0.046724 | 685 | 4 | −1.3221 | 0.99951 | 0.99953 | 1 | 17525 | 0 | −1.3221 |
| GRWD1 | 4 | 0.00049512 | 0.001786 | 0.046728 | 686 | 3 | −2.334 | 0.2091 | 0.39352 | 0.990803 | 7089 | 1 | −2.334 |
| PQBP1 | 4 | 0.0005017 | 0.0018107 | 0.047266 | 687 | 4 | −1.4597 | 0.99442 | 0.99453 | 1 | 16934 | 0 | −1.4597 |
| RBM17 | 4 | 0.00050235 | 0.0018118 | 0.047266 | 688 | 4 | −2.1806 | 0.9995 | 0.99952 | 1 | 17523 | 0 | −2.1806 |
| NAA20 | 4 | 0.00050543 | 0.0018255 | 0.047555 | 689 | 3 | −2.4164 | 0.12569 | 0.26623 | 0.938109 | 5056 | 1 | −2.4164 |
| AURKA | 4 | 0.00050729 | 0.001831 | 0.047592 | 690 | 4 | −1.705 | 0.99949 | 0.99951 | 1 | 17521 | 0 | −1.705 |
| WBSCR16 | 4 | 0.00050876 | 0.0018348 | 0.047592 | 691 | 4 | −1.7135 | 0.98557 | 0.98564 | 1 | 16520 | 0 | −1.7135 |
| TUT1 | 4 | 0.00050882 | 0.0018348 | 0.047592 | 692 | 4 | −1.556 | 0.99949 | 0.99951 | 1 | 17520 | 0 | −1.556 |
| CIAPIN1 | 4 | 0.00051342 | 0.0018524 | 0.047909 | 693 | 4 | −1.6801 | 0.99949 | 0.99951 | 1 | 17519 | 0 | −1.6801 |
| CDK6 | 4 | 0.00051884 | 0.0018716 | 0.048329 | 694 | 4 | −2.3633 | 0.99948 | 0.9995 | 1 | 17517 | 0 | −2.3633 |
| DUT | 4 | 0.00052126 | 0.0018809 | 0.048329 | 695 | 4 | −2.5564 | 0.99205 | 0.99218 | 1 | 16790 | 0 | −2.5564 |
| CENPP | 4 | 0.00052181 | 0.001882 | 0.048329 | 696 | 4 | −2.073 | 0.99921 | 0.9992 | 1 | 17431 | 0 | −2.073 |
| FANCG | 4 | 0.00052195 | 0.001882 | 0.048329 | 697 | 4 | −0.9392 | 0.99948 | 0.9995 | 1 | 17515 | 0 | −0.9392 |
| TOP1 | 4 | 0.00052468 | 0.0018924 | 0.048528 | 698 | 4 | −2.1419 | 0.99948 | 0.9995 | 1 | 17514 | 0 | −2.1419 |
| UBA52 | 3 | 0.00052947 | 0.0015578 | 0.043195 | 699 | 3 | −3.832 | 0.99947 | 0.99947 | 1 | 17513 | 0 | −3.832 |
| DLST | 4 | 0.00053276 | 0.0019182 | 0.049119 | 700 | 4 | −1.5659 | 0.99947 | 0.99949 | 1 | 17512 | 0 | −1.5659 |
| TUBB4B | 4 | 0.00053494 | 0.0019253 | 0.049176 | 701 | 4 | −0.87969 | 0.99947 | 0.99948 | 1 | 17510 | 0 | −0.87969 |
| SUPT16H | 4 | 0.0005355 | 0.0019258 | 0.049176 | 702 | 4 | −2.9335 | 0.9988 | 0.99879 | 1 | 17348 | 0 | −2.9335 |
| TMA16 | 4 | 0.00053693 | 0.0019308 | 0.049232 | 703 | 4 | −1.2658 | 0.99946 | 0.99948 | 1 | 17509 | 0 | −1.2658 |
| EIF1AD | 4 | 0.00053896 | 0.0019341 | 0.049247 | 704 | 4 | −1.8609 | 0.99867 | 0.99868 | 1 | 17318 | 0 | −1.8609 |
| GSK3A | 4 | 0.00054293 | 0.0019511 | 0.049526 | 705 | 4 | −1.5734 | 0.99946 | 0.99948 | 1 | 17507 | 0 | −1.5734 |
| CNOT7 | 4 | 0.00054331 | 0.0019516 | 0.049526 | 706 | 3 | −1.8081 | 0.95598 | 0.95584 | 1 | 15816 | 0 | −1.8081 |
| VRK1 | 4 | 0.00054374 | 0.0019533 | 0.049526 | 707 | 3 | −1.4744 | 0.19835 | 0.37782 | 0.987754 | 6836 | 1 | −1.4744 |
| RPIA | 4 | 0.00054636 | 0.001967 | 0.049804 | 708 | 3 | −3.1559 | 0.91022 | 0.90999 | 1 | 14956 | 0 | −3.1559 |
| MCM3 | 4 | 0.00054987 | 0.0019823 | 0.050122 | 709 | 3 | −1.6555 | 0.93705 | 0.9369 | 1 | 15441 | 0 | −1.6555 |
| GEMIN8 | 4 | 0.00055141 | 0.0019873 | 0.050177 | 710 | 4 | −1.8524 | 0.99945 | 0.99947 | 1 | 17505 | 0 | −1.8524 |
| POLE2 | 4 | 0.00055427 | 0.0019982 | 0.050327 | 711 | 4 | −2.7005 | 0.99832 | 0.99834 | 1 | 17261 | 0 | −2.7005 |
| RAE1 | 4 | 0.00055446 | 0.0019988 | 0.050327 | 712 | 4 | −1.0511 | 0.99945 | 0.99946 | 1 | 17504 | 0 | −1.0511 |
| SUPV3L1 | 4 | 0.00055548 | 0.002004 | 0.050338 | 713 | 4 | −1.9248 | 0.99944 | 0.99946 | 1 | 17503 | 0 | −1.9248 |
| CFDP1 | 4 | 0.00055559 | 0.0020048 | 0.050338 | 714 | 4 | −1.651 | 0.99812 | 0.99816 | 1 | 17233 | 0 | −1.651 |
| SMCIA | 4 | 0.00055651 | 0.0020092 | 0.050378 | 715 | 4 | −1.8933 | 0.99944 | 0.99946 | 1 | 17502 | 0 | −1.8933 |
| PAK1IP1 | 4 | 0.00055869 | 0.0020191 | 0.050555 | 716 | 4 | −2.5457 | 0.99872 | 0.99872 | 1 | 17329 | 0 | −2.5457 |
| TOMM20 | 4 | 0.00055958 | 0.0020235 | 0.050594 | 717 | 3 | −1.3401 | 0.95595 | 0.95581 | 1 | 15814 | 0 | −1.3401 |
| RBX1 | 4 | 0.00056143 | 0.0020339 | 0.050715 | 718 | 4 | −1.6434 | 0.99944 | 0.99946 | 1 | 17501 | 0 | −1.6434 |
| GTF2H3 | 4 | 0.0005637 | 0.0020416 | 0.050835 | 719 | 4 | −1.2466 | 0.99944 | 0.99946 | 1 | 17500 | 0 | −1.2466 |
| WDR74 | 4 | 0.00056432 | 0.0020443 | 0.050835 | 720 | 4 | −2.6884 | 0.99944 | 0.99946 | 1 | 17499 | 0 | −2.6884 |
| ALG9 | 4 | 0.00056627 | 0.0020553 | 0.051037 | 721 | 4 | −1.7685 | 0.99921 | 0.9992 | 1 | 17428 | 0 | −1.7685 |
| PSMC5 | 4 | 0.00057076 | 0.0020717 | 0.051324 | 722 | 4 | −1.7576 | 0.99943 | 0.99945 | 1 | 17497 | 0 | −1.7576 |
| PCNP | 3 | 0.00057097 | 0.0016823 | 0.04533 | 723 | 3 | −1.3995 | 0.99943 | 0.99944 | 1 | 17496 | 0 | −1.3995 |
| PET112 | 4 | 0.00057138 | 0.0020745 | 0.051324 | 724 | 4 | −1.3177 | 0.99943 | 0.99945 | 1 | 17495 | 0 | −1.3177 |
| MRPL49 | 4 | 0.00057256 | 0.0020761 | 0.051324 | 725 | 3 | −1.3896 | 0.31617 | 0.49932 | 1 | 8702 | 1 | −1.3896 |
| LSM4 | 4 | 0.00057368 | 0.0020789 | 0.051324 | 726 | 4 | −1.2294 | 0.99943 | 0.99945 | 1 | 17494 | 0 | −1.2294 |
| ATP6V1E1 | 4 | 0.0005741 | 0.0020811 | 0.051324 | 727 | 4 | −2.1806 | 0.99943 | 0.99945 | 1 | 17493 | 0 | −2.1806 |
| DSCC1 | 4 | 0.00057663 | 0.0020909 | 0.051497 | 728 | 4 | −1.5564 | 0.99922 | 0.99921 | 1 | 17433 | 0 | −1.5564 |
| RNF8 | 4 | 0.00057725 | 0.002095 | 0.051528 | 729 | 4 | −1.0026 | 0.99942 | 0.99944 | 1 | 17492 | 0 | −1.0026 |
| KAT2A | 4 | 0.00057914 | 0.002103 | 0.051654 | 730 | 4 | −0.90785 | 0.99942 | 0.99944 | 1 | 17491 | 0 | −0.90785 |
| RPS8 | 3 | 0.00058064 | 0.0017125 | 0.045759 | 731 | 3 | −4.5932 | 0.99942 | 0.99943 | 1 | 17490 | 0 | −4.5932 |
| ZNF622 | 4 | 0.00058443 | 0.0021211 | 0.052027 | 732 | 4 | −1.732 | 0.99942 | 0.99944 | 1 | 17489 | 0 | −1.732 |
| COX19 | 4 | 0.00058868 | 0.0021326 | 0.052239 | 733 | 4 | −1.6425 | 0.99941 | 0.99943 | 1 | 17488 | 0 | −1.6425 |
| TAF11 | 4 | 0.0005927 | 0.0021463 | 0.052503 | 734 | 2 | −0.12957 | 0.79265 | 0.82396 | 1 | 13490 | 1 | −0.12957 |
| EIF2B3 | 4 | 0.0005949 | 0.0021556 | 0.052581 | 735 | 4 | −1.506 | 0.99941 | 0.99943 | 1 | 17487 | 0 | −1.506 |
| WBSCR22 | 4 | 0.00059586 | 0.00216 | 0.052581 | 736 | 4 | −2.4087 | 0.99924 | 0.99925 | 1 | 17438 | 0 | −2.4087 |
| POLE3 | 4 | 0.00059662 | 0.0021611 | 0.052581 | 737 | 4 | −1.8615 | 0.9994 | 0.99942 | 1 | 17486 | 0 | −1.8615 |
| DIS3 | 4 | 0.00059911 | 0.0021699 | 0.052679 | 738 | 4 | −2.5053 | 0.96949 | 0.96939 | 1 | 16112 | 0 | −2.5053 |
| PSMF1 | 4 | 0.00059964 | 0.002171 | 0.052679 | 739 | 4 | −1.7119 | 0.9994 | 0.99942 | 1 | 17485 | 0 | −1.7119 |
| SKP1 | 3 | 0.00059971 | 0.0017646 | 0.046506 | 740 | 3 | −2.3501 | 0.9994 | 0.99942 | 1 | 17484 | 0 | −2.3501 |
| YARS2 | 4 | 0.00060398 | 0.0021896 | 0.05306 | 741 | 4 | −3.6474 | 0.9994 | 0.99942 | 1 | 17483 | 0 | −3.6474 |
| NVL | 4 | 0.00060892 | 0.0022077 | 0.053427 | 742 | 4 | −3.5027 | 0.99908 | 0.99906 | 1 | 17406 | 0 | −3.5027 |
| IPO13 | 4 | 0.00061363 | 0.0022291 | 0.053872 | 743 | 4 | −1.4439 | 0.99817 | 0.99821 | 1 | 17236 | 0 | −1.4439 |
| ASCC3 | 4 | 0.00061692 | 0.0022423 | 0.054118 | 744 | 4 | −1.1221 | 0.99938 | 0.9994 | 1 | 17482 | 0 | −1.1221 |
| LONP1 | 4 | 0.00062916 | 0.0022774 | 0.054811 | 745 | 4 | −1.7214 | 0.99937 | 0.99939 | 1 | 17479 | 0 | −1.7214 |
| C15orf52 | 4 | 0.00063006 | 0.0022801 | 0.054811 | 746 | 4 | −0.88202 | 0.99937 | 0.99939 | 1 | 17478 | 0 | −0.88202 |
| NSUN4 | 4 | 0.00063592 | 0.0023037 | 0.055305 | 747 | 4 | −1.2288 | 0.99936 | 0.99938 | 1 | 17477 | 0 | −1.2288 |
| RPLP2 | 3 | 0.00063689 | 0.0018765 | 0.048329 | 748 | 3 | −1.9163 | 0.99936 | 0.99938 | 1 | 17476 | 0 | −1.9163 |
| MVK | 4 | 0.00063752 | 0.0023114 | 0.055415 | 749 | 4 | −2.2041 | 0.98057 | 0.98065 | 1 | 16371 | 0 | −2.2041 |
| MRPL18 | 4 | 0.00063954 | 0.0023202 | 0.055468 | 750 | 4 | −0.9597 | 0.99936 | 0.99938 | 1 | 17475 | 0 | −0.9597 |
| STT3B | 4 | 0.00063995 | 0.0023213 | 0.055468 | 751 | 4 | −1.1927 | 0.98732 | 0.98741 | 1 | 16591 | 0 | −1.1927 |
| ILF2 | 4 | 0.00064205 | 0.002329 | 0.055468 | 752 | 4 | −2.2415 | 0.99936 | 0.99937 | 1 | 17474 | 0 | −2.2415 |
| GCN1L1 | 4 | 0.00064286 | 0.0023322 | 0.055468 | 753 | 4 | −1.9779 | 0.99702 | 0.99705 | 1 | 17116 | 0 | −1.9779 |
| DNLZ | 4 | 0.00064383 | 0.0023361 | 0.055468 | 754 | 4 | −1.6336 | 0.9991 | 0.99908 | 1 | 17409 | 0 | −1.6336 |
| SRPR | 4 | 0.00064465 | 0.0023383 | 0.055468 | 755 | 3 | −1.5627 | 0.64523 | 0.7584 | 1 | 12490 | 1 | −1.5627 |
| NAPG | 4 | 0.00064547 | 0.0023405 | 0.055468 | 756 | 4 | −2.2546 | 0.99935 | 0.99937 | 1 | 17473 | 0 | −2.2546 |
| EIF2B2 | 4 | 0.00064638 | 0.0023421 | 0.055468 | 757 | 4 | −2.3479 | 0.99935 | 0.99937 | 1 | 17472 | 0 | −2.3479 |
| RIOK1 | 4 | 0.00064684 | 0.0023443 | 0.055468 | 758 | 4 | −2.3089 | 0.99935 | 0.99937 | 1 | 17471 | 0 | −2.3089 |
| CDK13 | 4 | 0.00064799 | 0.0023503 | 0.055538 | 759 | 4 | −1.9403 | 0.99935 | 0.99937 | 1 | 17470 | 0 | −1.9403 |
| KTI12 | 4 | 0.00064913 | 0.0023553 | 0.055581 | 760 | 4 | −1.1404 | 0.99844 | 0.99847 | 1 | 17278 | 0 | −1.1404 |
| PSMA3 | 4 | 0.00065117 | 0.0023613 | 0.055591 | 761 | 4 | −2.4797 | 0.9922 | 0.99233 | 1 | 16801 | 0 | −2.4797 |
| MCM4 | 4 | 0.0006512 | 0.0023619 | 0.055591 | 762 | 4 | −1.2314 | 0.99935 | 0.99937 | 1 | 17469 | 0 | −1.2314 |
| SPINT1 | 4 | 0.00065906 | 0.0023937 | 0.056206 | 763 | 4 | −0.95708 | 0.99934 | 0.99936 | 1 | 17468 | 0 | −0.95708 |
| HARS2 | 4 | 0.0006593 | 0.0023942 | 0.056206 | 764 | 4 | −1.8839 | 0.99934 | 0.99936 | 1 | 17467 | 0 | −1.8839 |
| NCAPH2 | 4 | 0.00066092 | 0.0024024 | 0.056283 | 765 | 4 | −1.1466 | 0.99934 | 0.99935 | 1 | 17466 | 0 | −1.1466 |
| RPA1 | 4 | 0.00066162 | 0.0024046 | 0.056283 | 766 | 4 | −2.2589 | 0.99934 | 0.99935 | 1 | 17465 | 0 | −2.2589 |
| UXT | 4 | 0.00066232 | 0.0024068 | 0.056283 | 767 | 4 | −1.9861 | 0.99934 | 0.99935 | 1 | 17464 | 0 | −1.9861 |
| RAD9A | 4 | 0.00066451 | 0.0024145 | 0.056359 | 768 | 4 | −2.307 | 0.9817 | 0.98177 | 1 | 16404 | 0 | −2.307 |
| OGFR | 4 | 0.00066559 | 0.0024189 | 0.056359 | 769 | 4 | −0.81096 | 0.99933 | 0.99935 | 1 | 17463 | 0 | −0.81096 |
| TOMM40 | 4 | 0.00066582 | 0.0024194 | 0.056359 | 770 | 4 | −1.8101 | 0.99933 | 0.99935 | 1 | 17462 | 0 | −1.8101 |
| DDX6 | 4 | 0.00067752 | 0.002465 | 0.057345 | 771 | 4 | −2.3828 | 0.99919 | 0.99918 | 1 | 17425 | 0 | −2.3828 |
| TRAPPC11 | 4 | 0.00068738 | 0.0025061 | 0.058192 | 772 | 4 | −2.471 | 0.99931 | 0.99933 | 1 | 17461 | 0 | −2.471 |
| SPCS2 | 4 | 0.00068815 | 0.0025094 | 0.058192 | 773 | 4 | −2.2527 | 0.98398 | 0.98404 | 1 | 16472 | 0 | −2.2527 |
| RPAIN | 4 | 0.00068866 | 0.002511 | 0.058192 | 774 | 4 | −1.7049 | 0.9929 | 0.99302 | 1 | 16839 | 0 | −1.7049 |
| RPS19 | 3 | 0.00069135 | 0.0020262 | 0.050594 | 775 | 3 | −3.0518 | 0.99931 | 0.99931 | 1 | 17460 | 0 | −3.0518 |
| RBM25 | 4 | 0.00069605 | 0.0025368 | 0.058652 | 776 | 4 | −2.1991 | 0.9993 | 0.99932 | 1 | 17459 | 0 | −2.1991 |
| GTF3C1 | 4 | 0.00069629 | 0.0025374 | 0.058652 | 777 | 4 | −1.3316 | 0.9993 | 0.99932 | 1 | 17458 | 0 | −1.3316 |
| ENY2 | 4 | 0.00069762 | 0.0025428 | 0.058703 | 778 | 3 | −1.7654 | 0.60365 | 0.74287 | 1 | 12239 | 1 | −1.7654 |
| GSG2 | 4 | 0.00070041 | 0.0025511 | 0.058818 | 779 | 4 | −2.0652 | 0.9993 | 0.99931 | 1 | 17457 | 0 | −2.0652 |
| NOP2 | 4 | 0.00070285 | 0.0025598 | 0.058945 | 780 | 4 | −1.5168 | 0.9993 | 0.99931 | 1 | 17456 | 0 | −1.5168 |
| PDCD10 | 4 | 0.00070529 | 0.0025708 | 0.059122 | 781 | 4 | −1.2285 | 0.99929 | 0.99931 | 1 | 17455 | 0 | −1.2285 |
| DEXI | 4 | 0.00070773 | 0.0025779 | 0.059211 | 782 | 4 | −1.0973 | 0.99929 | 0.9993 | 1 | 17454 | 0 | −1.0973 |
| BCL2L1 | 4 | 0.00070969 | 0.0025823 | 0.059236 | 783 | 4 | −3.717 | 0.99929 | 0.9993 | 1 | 17453 | 0 | −3.717 |
| ELL | 4 | 0.00071141 | 0.0025906 | 0.059324 | 784 | 4 | −1.7749 | 0.99929 | 0.9993 | 1 | 17452 | 0 | −1.7749 |
| MCRS1 | 4 | 0.00071234 | 0.0025928 | 0.059324 | 785 | 4 | −1.5183 | 0.96124 | 0.96112 | 1 | 15927 | 0 | −1.5183 |
| PPIE | 4 | 0.00071462 | 0.0025982 | 0.059375 | 786 | 4 | −1.5753 | 0.99929 | 0.99929 | 1 | 17450 | 0 | −1.5753 |
| MVD | 4 | 0.00071573 | 0.0026021 | 0.059387 | 787 | 3 | −2.2277 | 0.066827 | 0.16731 | 0.843568 | 3530 | 1 | −2.2277 |
| SERBP1 | 4 | 0.00072629 | 0.0026383 | 0.060137 | 788 | 4 | −1.4511 | 0.99927 | 0.99928 | 1 | 17449 | 0 | −1.4511 |
| PNN | 4 | 0.00072979 | 0.0026498 | 0.060324 | 789 | 4 | −1.159 | 0.99927 | 0.99928 | 1 | 17448 | 0 | −1.159 |
| ILK | 4 | 0.00073223 | 0.0026608 | 0.060497 | 790 | 4 | −2.5597 | 0.98695 | 0.98703 | 1 | 16567 | 0 | −2.5597 |
| LETM1 | 3 | 0.00073667 | 0.0021529 | 0.052581 | 791 | 3 | −2.0544 | 0.99926 | 0.99927 | 1 | 17447 | 0 | −2.0544 |
| YBX1 | 4 | 0.00073734 | 0.00268 | 0.060857 | 792 | 4 | −1.3243 | 0.99926 | 0.99927 | 1 | 17446 | 0 | −1.3243 |
| TSC2 | 4 | 0.00074088 | 0.0026915 | 0.061042 | 793 | 4 | −1.6204 | 0.99926 | 0.99926 | 1 | 17445 | 0 | −1.6204 |
| RRM1 | 4 | 0.00074342 | 0.0026981 | 0.061108 | 794 | 4 | −2.6187 | 0.99926 | 0.99926 | 1 | 17444 | 0 | −2.6187 |
| SS18L2 | 4 | 0.00074546 | 0.0027041 | 0.061108 | 795 | 4 | −1.6365 | 0.99925 | 0.99926 | 1 | 17443 | 0 | −1.6365 |
| DCLRE1B | 4 | 0.00074699 | 0.0027079 | 0.061108 | 796 | 4 | −1.4202 | 0.99925 | 0.99926 | 1 | 17442 | 0 | −1.4202 |
| RPS13 | 4 | 0.00074724 | 0.0027079 | 0.061108 | 797 | 4 | −2.8872 | 0.99925 | 0.99926 | 1 | 17441 | 0 | −2.8872 |
| MTG1 | 4 | 0.00075005 | 0.0027156 | 0.061204 | 798 | 4 | −0.75399 | 0.99925 | 0.99925 | 1 | 17440 | 0 | −0.75399 |
| NDUFAF1 | 4 | 0.00075478 | 0.0027326 | 0.061511 | 799 | 3 | −3.2341 | 0.86898 | 0.87329 | 1 | 14327 | 1 | −3.2341 |
| COX17 | 4 | 0.00075647 | 0.0027381 | 0.061558 | 800 | 4 | −1.277 | 0.99924 | 0.99925 | 1 | 17439 | 0 | −1.277 |
| SEPHS1 | 4 | 0.00075966 | 0.0027485 | 0.061612 | 801 | 4 | −1.8612 | 0.99231 | 0.99243 | 1 | 16809 | 0 | −1.8612 |
| GINS1 | 4 | 0.0007602 | 0.0027502 | 0.061612 | 802 | 3 | −2.1136 | 0.94328 | 0.94313 | 1 | 15559 | 0 | −2.1136 |
| CHMP7 | 4 | 0.00076129 | 0.0027534 | 0.061612 | 803 | 3 | −1.1516 | 0.85041 | 0.8595 | 1 | 14107 | 1 | −1.1516 |
| ATRX | 4 | 0.00076345 | 0.00276 | 0.061612 | 804 | 4 | −2.5208 | 0.99924 | 0.99924 | 1 | 17437 | 0 | −2.5208 |
| LIPT2 | 4 | 0.0007639 | 0.0027617 | 0.061612 | 805 | 4 | −1.8874 | 0.99918 | 0.99916 | 1 | 17422 | 0 | −1.8874 |
| PRELID1 | 4 | 0.00076455 | 0.0027639 | 0.061612 | 806 | 3 | −1.7103 | 0.90978 | 0.90951 | 1 | 14946 | 0 | −1.7103 |
| NAT10 | 4 | 0.00076474 | 0.0027644 | 0.061612 | 807 | 4 | −2.2606 | 0.99924 | 0.99924 | 1 | 17436 | 0 | −2.2606 |
| AASDHPPT | 4 | 0.00076969 | 0.0027836 | 0.061964 | 808 | 4 | −1.7339 | 0.99923 | 0.99923 | 1 | 17435 | 0 | −1.7339 |
| ERAL1 | 4 | 0.00077882 | 0.0028193 | 0.062651 | 809 | 4 | −2.0518 | 0.99768 | 0.9977 | 1 | 17190 | 0 | −2.0518 |
| UBQLN4 | 4 | 0.00077965 | 0.0028215 | 0.062651 | 810 | 4 | −1.5936 | 0.99922 | 0.99921 | 1 | 17434 | 0 | −1.5936 |
| FAM210A | 4 | 0.00078998 | 0.002862 | 0.063475 | 811 | 4 | −1.241 | 0.99921 | 0.9992 | 1 | 17432 | 0 | −1.241 |
| PDSS1 | 4 | 0.00079493 | 0.0028796 | 0.063785 | 812 | 3 | −1.8459 | 0.73123 | 0.79333 | 1 | 13034 | 1 | −1.8459 |
| DARS2 | 4 | 0.00079987 | 0.0029004 | 0.064168 | 813 | 4 | −1.1067 | 0.9992 | 0.99919 | 1 | 17427 | 0 | −1.1067 |
| SEC61G | 3 | 0.00080389 | 0.0023432 | 0.055468 | 814 | 3 | −1.1814 | 0.9992 | 0.9992 | 1 | 17426 | 0 | −1.1814 |
| CDC37 | 4 | 0.00081256 | 0.002947 | 0.06512 | 815 | 4 | −2.3433 | 0.99919 | 0.99918 | 1 | 17424 | 0 | −2.3433 |
| RPAP1 | 4 | 0.00081965 | 0.0029756 | 0.06567 | 816 | 4 | −1.9588 | 0.99918 | 0.99917 | 1 | 17423 | 0 | −1.9588 |
| GFM1 | 4 | 0.00082623 | 0.0029964 | 0.065993 | 817 | 4 | −1.156 | 0.99917 | 0.99916 | 1 | 17421 | 0 | −1.156 |
| CDK5RAP3 | 4 | 0.00082651 | 0.0029975 | 0.065993 | 818 | 4 | −0.86133 | 0.99917 | 0.99916 | 1 | 17420 | 0 | −0.86133 |
| GRID2IP | 4 | 0.00083009 | 0.0030085 | 0.066073 | 819 | 4 | −0.98817 | 0.99917 | 0.99916 | 1 | 17419 | 0 | −0.98817 |
| FANCF | 4 | 0.00083241 | 0.0030167 | 0.066173 | 820 | 4 | −1.3792 | 0.99812 | 0.99816 | 1 | 17232 | 0 | −1.3792 |
| MRPL12 | 4 | 0.00083535 | 0.0030266 | 0.066309 | 821 | 4 | −1.111 | 0.99916 | 0.99915 | 1 | 17418 | 0 | −1.111 |
| CENPQ | 4 | 0.00083707 | 0.0030321 | 0.066349 | 822 | 4 | −2.7291 | 0.98884 | 0.98898 | 1 | 16644 | 0 | −2.7291 |
| CWC25 | 4 | 0.00084426 | 0.0030589 | 0.066856 | 823 | 4 | −1.586 | 0.99916 | 0.99914 | 1 | 17417 | 0 | −1.586 |
| PDCD6IP | 4 | 0.00084763 | 0.0030754 | 0.06134 | 824 | 4 | −1.2028 | 0.99915 | 0.99914 | 1 | 17416 | 0 | −1.2028 |
| NCAPD2 | 4 | 0.000851 | 0.003085 | 0.067262 | 825 | 4 | −1.2863 | 0.99915 | 0.99914 | 1 | 17415 | 0 | −1.2863 |
| MAGOH | 4 | 0.00086156 | 0.0031253 | 0.068059 | 826 | 4 | −2.9645 | 0.99378 | 0.99388 | 1 | 16886 | 0 | −2.9645 |
| SELRC1 | 4 | 0.00086333 | 0.0031341 | 0.068168 | 827 | 3 | −1.7189 | 0.14558 | 0.29784 | 0.952116 | 5565 | 1 | −1.7189 |
| DDX51 | 4 | 0.00086747 | 0.00315 | 0.068431 | 828 | 4 | −3.0524 | 0.99067 | 0.99076 | 1 | 16721 | 0 | −3.0524 |
| CUX1 | 4 | 0.00086925 | 0.0031565 | 0.068481 | 829 | 4 | −2.0939 | 0.99634 | 0.99641 | 1 | 17062 | 0 | −2.0939 |
| ZC3H3 | 4 | 0.00087029 | 0.0031598 | 0.068481 | 830 | 4 | −1.3539 | 0.99913 | 0.99912 | 1 | 17414 | 0 | −1.3539 |
| EMC6 | 4 | 0.00087639 | 0.0031818 | 0.068874 | 831 | 3 | −1.0314 | 0.5512 | 0.69743 | 1 | 11549 | 1 | −1.0314 |
| NOB1 | 4 | 0.00087861 | 0.0031867 | 0.068898 | 832 | 4 | −3.1791 | 0.99912 | 0.99911 | 1 | 17413 | 0 | −3.1791 |
| RICTOR | 4 | 0.00088092 | 0.0031966 | 0.069029 | 833 | 4 | −1.6557 | 0.99912 | 0.99911 | 1 | 17412 | 0 | −1.6557 |
| UBE2O | 4 | 0.00088584 | 0.0032141 | 0.069325 | 834 | 4 | −1.4054 | 0.99911 | 0.9991 | 1 | 17411 | 0 | −1.4054 |
| UAP1 | 4 | 0.00088716 | 0.0032207 | 0.069384 | 835 | 3 | −1.4867 | 0.64437 | 0.75807 | 1 | 12484 | 1 | −1.4867 |
| NME6 | 4 | 0.00088897 | 0.0032278 | 0.069431 | 836 | 4 | −1.8232 | 0.99822 | 0.99825 | 1 | 17243 | 0 | −1.8232 |
| RFWD2 | 4 | 0.00089019 | 0.0032306 | 0.069431 | 837 | 4 | −2.2616 | 0.99911 | 0.9991 | 1 | 17410 | 0 | −2.2616 |
| EXOSC2 | 4 | 0.00089319 | 0.0032421 | 0.069595 | 838 | 3 | −2.0668 | 0.80955 | 0.83356 | 1 | 13655 | 1 | −2.0668 |
| TBCE | 4 | 0.00089439 | 0.003247 | 0.069619 | 839 | 3 | −2.2373 | 0.44751 | 0.60933 | 1 | 10242 | 1 | −2.2373 |
| EXOSC10 | 4 | 0.00090124 | 0.0032717 | 0.070064 | 840 | 4 | −1.5667 | 0.99305 | 0.99316 | 1 | 16847 | 0 | −1.5667 |
| DYNC1I2 | 4 | 0.00090542 | 0.003286 | 0.070286 | 841 | 4 | −2.3272 | 0.99909 | 0.99908 | 1 | 17408 | 0 | −2.3272 |
| WDR24 | 4 | 0.00090836 | 0.0032937 | 0.070367 | 842 | 3 | −1.6104 | 0.86821 | 0.87269 | 1 | 14318 | 1 | −1.6104 |
| NDUFS2 | 4 | 0.00091558 | 0.0033205 | 0.070809 | 843 | 4 | −1.9904 | 0.99494 | 0.99504 | 1 | 16965 | 0 | −1.9904 |
| ARMC7 | 4 | 0.00091578 | 0.0033222 | 0.070809 | 844 | 4 | −1.6499 | 0.99908 | 0.99907 | 1 | 17407 | 0 | −1.6499 |
| TSEN15 | 4 | 0.00091764 | 0.003331 | 0.070912 | 845 | 3 | −1.1324 | 0.30944 | 0.4937 | 1 | 8618 | 1 | −1.1324 |
| ATIC | 4 | 0.0009197 | 0.0033403 | 0.071027 | 846 | 3 | −2.9017 | 0.79051 | 0.82277 | 1 | 13467 | 1 | −2.9017 |
| IMP3 | 4 | 0.00092563 | 0.0033661 | 0.071491 | 847 | 4 | −1.246 | 0.99907 | 0.99906 | 1 | 17405 | 0 | −1.246 |
| KANSL3 | 4 | 0.00092866 | 0.0033754 | 0.071521 | 848 | 4 | −2.5444 | 0.99101 | 0.99112 | 1 | 16740 | 0 | −2.5444 |
| MNAT1 | 4 | 0.00093013 | 0.003382 | 0.07155 | 849 | 4 | −1.5315 | 0.99907 | 0.99905 | 1 | 17404 | 0 | −1.5315 |
| CRTC2 | 4 | 0.00093164 | 0.0033847 | 0.07155 | 850 | 4 | −2.682 | 0.99907 | 0.99905 | 1 | 17403 | 0 | −2.682 |
| MBD3 | 4 | 0.00094009 | 0.0034099 | 0.071805 | 851 | 4 | −0.999 | 0.99906 | 0.99904 | 1 | 17402 | 0 | −0.999 |
| METTL16 | 4 | 0.00094047 | 0.0034116 | 0.071805 | 852 | 3 | −2.7693 | 0.93827 | 0.93815 | 1 | 15472 | 0 | −2.7693 |
| VAC14 | 4 | 0.00094131 | 0.0034127 | 0.071805 | 853 | 4 | −0.89677 | 0.99906 | 0.99904 | 1 | 17401 | 0 | −0.89677 |
| YEATS4 | 4 | 0.00094343 | 0.0034225 | 0.071929 | 854 | 4 | −1.611 | 0.99906 | 0.99904 | 1 | 17400 | 0 | −1.611 |
| KIAA1524 | 4 | 0.00094495 | 0.0034288 | 0.071978 | 855 | 4 | −1.4931 | 0.99906 | 0.99904 | 1 | 17399 | 0 | −1.4931 |
| LARS | 4 | 0.00094769 | 0.003439 | 0.072107 | 856 | 4 | −3.0806 | 0.99905 | 0.99904 | 1 | 17398 | 0 | −3.0806 |
| NIP7 | 4 | 0.00095105 | 0.0034516 | 0.072288 | 857 | 4 | −2.2191 | 0.99905 | 0.99903 | 1 | 17397 | 0 | −2.2191 |
| ADSS | 4 | 0.00095504 | 0.003467 | 0.072525 | 858 | 4 | −1.57 | 0.99904 | 0.99903 | 1 | 17396 | 0 | −1.57 |
| NARS2 | 4 | 0.00095933 | 0.0034757 | 0.072624 | 859 | 4 | −1.6004 | 0.97201 | 0.97197 | 1 | 16169 | 0 | −1.6004 |
| NFYC | 4 | 0.00096829 | 0.0035026 | 0.073101 | 860 | 4 | −1.8369 | 0.99903 | 0.99901 | 1 | 17395 | 0 | −1.8369 |
| GINS3 | 4 | 0.00097046 | 0.0035097 | 0.073166 | 861 | 4 | −1.3368 | 0.99903 | 0.99901 | 1 | 17394 | 0 | −1.3368 |
| RABIF | 4 | 0.00097295 | 0.0035152 | 0.073195 | 862 | 4 | −1.58 | 0.99903 | 0.99901 | 1 | 17392 | 0 | −1.58 |
| EIF5A | 2 | 0.0009788 | 0.0018386 | 0.047623 | 863 | 2 | −2.686 | 0.99902 | 0.99899 | 1 | 17390 | 0 | −2.686 |
| NOP56 | 4 | 0.00098138 | 0.0035366 | 0.073556 | 864 | 4 | −2.1521 | 0.99902 | 0.999 | 1 | 17389 | 0 | −2.1521 |
| TBCC | 4 | 0.00098702 | 0.0035618 | 0.073956 | 865 | 4 | −1.6457 | 0.99901 | 0.99899 | 1 | 17388 | 0 | −1.6457 |
| WDR18 | 4 | 0.00098768 | 0.003564 | 0.073956 | 866 | 3 | −3.1328 | 0.74499 | 0.7997 | 1 | 13121 | 1 | −3.1328 |
| PTAR1 | 4 | 0.00098937 | 0.0035701 | 0.073979 | 867 | 3 | −2.0962 | 0.82975 | 0.8457 | 1 | 13865 | 1 | −2.0962 |
| NFE2L2 | 4 | 0.00099112 | 0.0035734 | 0.073979 | 868 | 4 | −2.5571 | 0.99901 | 0.99899 | 1 | 17387 | 0 | −2.5571 |
| PMPCB | 4 | 0.00099459 | 0.003586 | 0.074138 | 869 | 4 | −1.0994 | 0.99901 | 0.99899 | 1 | 17386 | 0 | −1.0994 |
| COX7C | 4 | 0.00099554 | 0.0035893 | 0.074138 | 870 | 4 | −2.0206 | 0.999 | 0.99898 | 1 | 17385 | 0 | −2.0206 |
| CENPO | 4 | 0.0010056 | 0.0036288 | 0.074777 | 871 | 4 | −2.4989 | 0.99008 | 0.99016 | 1 | 16697 | 0 | −2.4989 |
| HARS | 4 | 0.0010063 | 0.0036315 | 0.074777 | 872 | 4 | −1.3687 | 0.99899 | 0.99898 | 1 | 17383 | 0 | −1.3687 |
| UBE2T | 4 | 0.0010069 | 0.0036326 | 0.074777 | 873 | 4 | −1.336 | 0.96881 | 0.96871 | 1 | 16096 | 0 | −1.336 |
| LINGO1 | 4 | 0.0010111 | 0.0036447 | 0.07494 | 874 | 4 | −1.2271 | 0.99899 | 0.99897 | 1 | 17382 | 0 | −1.2274 |
| MIPEP | 4 | 0.001018 | 0.0036743 | 0.075463 | 875 | 4 | −2.059 | 0.99826 | 0.99829 | 1 | 17252 | 0 | −2.059 |
| PDCD2 | 4 | 0.0010234 | 0.0036863 | 0.075624 | 876 | 4 | −1.71 | 0.99898 | 0.99897 | 1 | 17380 | 0 | −1.71 |
| FANCA | 4 | 0.001024 | 0.0036918 | 0.075651 | 877 | 3 | −1.4453 | 0.91742 | 0.91723 | 1 | 15070 | 0 | −1.4453 |
| RPS14 | 4 | 0.0010318 | 0.0037247 | 0.076167 | 878 | 4 | −1.2353 | 0.99897 | 0.99896 | 1 | 17378 | 0 | −1.2353 |
| MRPL50 | 4 | 0.0010324 | 0.0037275 | 0.076167 | 879 | 4 | −1.0539 | 0.99897 | 0.99896 | 1 | 17377 | 0 | −1.0539 |
| SYMPK | 4 | 0.0010393 | 0.0037527 | 0.076479 | 880 | 4 | −1.5469 | 0.99896 | 0.99895 | 1 | 17376 | 0 | −1.5469 |
| SSU72 | 4 | 0.0010396 | 0.0037538 | 0.076479 | 881 | 4 | −2.1095 | 0.99896 | 0.99895 | 1 | 17375 | 0 | −2.1095 |
| SKA2 | 4 | 0.0010406 | 0.0037576 | 0.076479 | 882 | 4 | −1.9971 | 0.99896 | 0.99895 | 1 | 17374 | 0 | −1.9971 |
| GRB2 | 4 | 0.0010432 | 0.0037648 | 0.076538 | 883 | 3 | −1.915 | 0.79251 | 0.82388 | 1 | 13487 | 1 | −1.915 |
| PYROXD1 | 4 | 0.0010519 | 0.0037999 | 0.077078 | 884 | 4 | −2.7306 | 0.96615 | 0.96605 | 1 | 16031 | 0 | −2.7306 |
| SLC25A19 | 4 | 0.001052 | 0.0037999 | 0.077078 | 885 | 3 | −2.1618 | 0.24978 | 0.44454 | 1 | 7898 | 1 | −2.1618 |
| MEAF6 | 4 | 0.0010653 | 0.0038465 | 0.077936 | 886 | 4 | −1.5771 | 0.99893 | 0.99893 | 1 | 17373 | 0 | −1.5771 |
| EHMT2 | 4 | 0.0010687 | 0.0038591 | 0.078103 | 887 | 4 | −0.70092 | 0.99893 | 0.99892 | 1 | 17372 | 0 | −0.70092 |
| DAZAP1 | 4 | 0.0010709 | 0.0038684 | 0.078205 | 888 | 4 | −2.4439 | 0.99743 | 0.99746 | 1 | 17164 | 0 | −2.4439 |
| TUBB | 4 | 0.001073 | 0.0038766 | 0.078283 | 889 | 4 | −1.8817 | 0.99893 | 0.99892 | 1 | 17371 | 0 | −1.8817 |
| ALG13 | 4 | 0.0010756 | 0.0038876 | 0.078417 | 890 | 3 | −1.9914 | 0.91314 | 0.91291 | 1 | 15000 | 0 | −1.9914 |
| GTF3C3 | 4 | 0.001077 | 0.0038953 | 0.07843 | 891 | 4 | −1.2396 | 0.99892 | 0.99891 | 1 | 17370 | 0 | −1.2396 |
| TCP1 | 4 | 0.0010777 | 0.0038969 | 0.07843 | 892 | 4 | −2.6231 | 0.98829 | 0.98838 | 1 | 16627 | 0 | −2.6231 |
| EIF3E | 4 | 0.0010794 | 0.0039041 | 0.078486 | 893 | 4 | −2.0137 | 0.99892 | 0.99891 | 1 | 17369 | 0 | −2.0137 |
| COPS2 | 4 | 0.0010838 | 0.0039189 | 0.078696 | 894 | 3 | −1.9057 | 0.87588 | 0.87869 | 1 | 14447 | 1 | −1.9057 |
| TAF2 | 4 | 0.0010893 | 0.0039364 | 0.07896 | 895 | 4 | −1.4434 | 0.99098 | 0.99109 | 1 | 16739 | 0 | −1.4434 |
| TRAIP | 4 | 0.0010949 | 0.0039545 | 0.079235 | 896 | 3 | −1.3967 | 0.81806 | 0.83857 | 1 | 13739 | 1 | −1.3967 |
| MRPL20 | 4 | 0.0011005 | 0.0039825 | 0.079707 | 897 | 3 | −0.68813 | 0.90112 | 0.90088 | 1 | 14804 | 1 | −0.68813 |
| NUP43 | 4 | 0.0011041 | 0.003994 | 0.079816 | 898 | 4 | −2.6617 | 0.9989 | 0.99889 | 1 | 17368 | 0 | −2.6617 |
| C3orf38 | 4 | 0.0011052 | 0.0039968 | 0.079816 | 899 | 4 | −1.1155 | 0.99889 | 0.99888 | 1 | 17367 | 0 | −1.1155 |
| ORC3 | 4 | 0.0011076 | 0.0040039 | 0.079847 | 900 | 4 | −1.0735 | 0.99889 | 0.99888 | 1 | 17366 | 0 | −1.0735 |
| KIF18A | 4 | 0.0011083 | 0.0040072 | 0.079847 | 901 | 4 | −1.7281 | 0.99889 | 0.99888 | 1 | 17365 | 0 | −1.7281 |
| DDX10 | 4 | 0.0011136 | 0.0040269 | 0.080125 | 902 | 4 | −4.1488 | 0.99615 | 0.99623 | 1 | 17053 | 0 | −4.1488 |
| USP9X | 4 | 0.0011179 | 0.0040362 | 0.080125 | 903 | 4 | −1.1196 | 0.99888 | 0.99887 | 1 | 17364 | 0 | −1.1196 |
| MRPL2 | 4 | 0.0011193 | 0.0040428 | 0.080125 | 904 | 4 | −1.7372 | 0.99888 | 0.99887 | 1 | 17363 | 0 | −1.7372 |
| POLR2C | 4 | 0.0011203 | 0.0040456 | 0.080125 | 905 | 4 | −3.1419 | 0.99888 | 0.99887 | 1 | 17362 | 0 | −3.1419 |
| GABPA | 4 | 0.001121 | 0.0040467 | 0.080125 | 906 | 4 | −0.98715 | 0.99888 | 0.99887 | 1 | 17361 | 0 | −0.98715 |
| CKAP5 | 4 | 0.0011213 | 0.0040478 | 0.080125 | 907 | 4 | −3.0725 | 0.99888 | 0.99887 | 1 | 17360 | 0 | −3.0725 |
| CSTF3 | 4 | 0.0011234 | 0.0040576 | 0.080233 | 908 | 4 | −3.127 | 0.99888 | 0.99887 | 1 | 17359 | 0 | −3.127 |
| TPX2 | 4 | 0.0011328 | 0.0040829 | 0.080643 | 909 | 4 | −2.0197 | 0.99887 | 0.99886 | 1 | 17358 | 0 | −2.0197 |
| EPRS | 4 | 0.0011502 | 0.0041476 | 0.081832 | 910 | 3 | −1.5672 | 0.90857 | 0.90829 | 1 | 14927 | 0 | −1.5672 |
| TRIAP1 | 4 | 0.0011569 | 0.0041777 | 0.082337 | 911 | 2 | −0.78485 | 0.91547 | 0.91526 | 1 | 15038 | 0 | −0.78485 |
| MMGT1 | 4 | 0.0011603 | 0.0041947 | 0.082557 | 912 | 3 | −2.3632 | 0.85716 | 0.86442 | 1 | 14197 | 1 | −2.3632 |
| SOD2 | 4 | 0.001161 | 0.004198 | 0.082557 | 913 | 3 | −1.1092 | 0.55932 | 0.70444 | 1 | 11667 | 1 | −1.1092 |
| CHMP6 | 3 | 0.0011682 | 0.0033704 | 0.0715 | 914 | 3 | −3.1616 | 0.99883 | 0.99886 | 1 | 17356 | 0 | −3.1616 |
| ZFYVE20 | 4 | 0.0011689 | 0.0042238 | 0.082973 | 915 | 3 | −1.784 | 0.90853 | 0.90825 | 1 | 14925 | 0 | −1.784 |
| ATP5SL | 4 | 0.0011752 | 0.0042436 | 0.083221 | 916 | 4 | −1.9566 | 0.99882 | 0.99881 | 1 | 17355 | 0 | −1.9566 |
| MRPL41 | 4 | 0.0011763 | 0.004249 | 0.083221 | 917 | 4 | −1.3262 | 0.99882 | 0.99881 | 1 | 17354 | 0 | −1.3262 |
| C14orf166 | 4 | 0.0011775 | 0.0042507 | 0.083221 | 918 | 3 | −1.0643 | 0.92442 | 0.92424 | 1 | 15211 | 0 | −1.0643 |
| POLR2E | 4 | 0.0011784 | 0.0042573 | 0.083221 | 919 | 4 | −1.5076 | 0.99882 | 0.99881 | 1 | 17353 | 0 | −1.5076 |
| CPSF1 | 4 | 0.001179 | 0.0042595 | 0.083221 | 920 | 4 | −2.5409 | 0.9966 | 0.99666 | 1 | 17083 | 0 | −2.5409 |
| RNF40 | 4 | 0.0011826 | 0.0042682 | 0.083302 | 921 | 4 | −2.4548 | 0.96353 | 0.96343 | 1 | 15972 | 0 | −2.4548 |
| GART | 4 | 0.0011862 | 0.0042792 | 0.083426 | 922 | 4 | −1.7079 | 0.98 | 0.98011 | 1 | 16350 | 0 | −1.7079 |
| FOXA1 | 4 | 0.00119 | 0.004294 | 0.083588 | 923 | 4 | −1.4437 | 0.99881 | 0.9988 | 1 | 17352 | 0 | −1.4437 |
| DNAJA3 | 4 | 0.0011918 | 0.0042968 | 0.083588 | 924 | 4 | −1.7205 | 0.99881 | 0.9988 | 1 | 17350 | 0 | −1.7205 |
| C1D | 2 | 0.0011944 | 0.0022785 | 0.054811 | 925 | 2 | −2.6596 | 0.99881 | 0.99877 | 1 | 17349 | 0 | −2.6596 |
| NDUFB8 | 4 | 0.0011961 | 0.0043138 | 0.083749 | 926 | 4 | −1.4794 | 0.9988 | 0.99879 | 1 | 17347 | 0 | −1.4794 |
| YRDC | 4 | 0.0011965 | 0.0043143 | 0.083749 | 927 | 4 | −2.4008 | 0.9988 | 0.99879 | 1 | 17346 | 0 | −2.4008 |
| MCPH1 | 4 | 0.0012038 | 0.004345 | 0.084116 | 928 | 3 | −1.6572 | 0.40803 | 0.57612 | 1 | 9781 | 1 | −1.6572 |
| POLE | 4 | 0.0012042 | 0.0043467 | 0.084116 | 929 | 4 | −1.5639 | 0.9988 | 0.99879 | 1 | 17345 | 0 | −1.5639 |
| UBA6 | 4 | 0.0012045 | 0.0043472 | 0.084116 | 930 | 4 | −3.0706 | 0.96809 | 0.96797 | 1 | 16084 | 0 | −3.0706 |
| PRMT1 | 4 | 0.0012063 | 0.0043582 | 0.084238 | 931 | 4 | −2.6272 | 0.99879 | 0.99879 | 1 | 17344 | 0 | −2.6272 |
| PSME3 | 4 | 0.0012133 | 0.0043812 | 0.084502 | 932 | 3 | −1.8308 | 0.56255 | 0.70721 | 1 | 11699 | 1 | −1.8308 |
| PSMA4 | 4 | 0.0012151 | 0.0043883 | 0.084549 | 933 | 4 | −2.9737 | 0.99878 | 0.99878 | 1 | 17343 | 0 | −2.9737 |
| MPI | 4 | 0.0012181 | 0.0043966 | 0.084618 | 934 | 4 | −1.0399 | 0.99878 | 0.99878 | 1 | 17342 | 0 | −1.0399 |
| UBE3A | 4 | 0.0012196 | 0.0044026 | 0.084643 | 935 | 4 | −0.9351 | 0.99878 | 0.99877 | 1 | 17341 | 0 | −0.9351 |
| PGGT1B | 4 | 0.0012266 | 0.0044317 | 0.085112 | 936 | 4 | −1.7075 | 0.99877 | 0.99877 | 1 | 17340 | 0 | −1.7075 |
| VIPAS39 | 4 | 0.001228 | 0.0044383 | 0.085147 | 937 | 4 | −1.2453 | 0.99877 | 0.99876 | 1 | 17339 | 0 | −1.2453 |
| OSBP | 4 | 0.0012392 | 0.004492 | 0.085947 | 938 | 4 | −1.1291 | 0.99876 | 0.99875 | 1 | 17337 | 0 | −1.1291 |
| RAD51 | 4 | 0.0012396 | 0.0044936 | 0.085947 | 939 | 4 | −3.3345 | 0.99876 | 0.99875 | 1 | 17336 | 0 | −3.3345 |
| MED18 | 4 | 0.0012448 | 0.0045106 | 0.08617 | 940 | 4 | −2.2704 | 0.99876 | 0.99875 | 1 | 17335 | 0 | −2.2704 |
| NDUFA8 | 4 | 0.0012468 | 0.0045183 | 0.086225 | 941 | 3 | −1.2897 | 0.22936 | 0.42261 | 0.999114 | 7556 | 1 | −1.2897 |
| NELFCD | 4 | 0.00125 | 0.0045266 | 0.086291 | 942 | 4 | −2.3183 | 0.99875 | 0.99874 | 1 | 17334 | 0 | −2.3183 |
| NKAP | 4 | 0.0012617 | 0.0045606 | 0.086848 | 943 | 4 | −1.3346 | 0.99874 | 0.99873 | 1 | 17333 | 0 | −1.3346 |
| ANKDD1A | 4 | 0.0012636 | 0.004571 | 0.086954 | 944 | 4 | −0.99871 | 0.99874 | 0.99873 | 1 | 17332 | 0 | −0.99871 |
| MALSU1 | 4 | 0.0012672 | 0.0045841 | 0.087113 | 945 | 3 | −1.2583 | 0.64975 | 0.76004 | 1 | 12518 | 1 | −1.2583 |
| SNUPN | 4 | 0.001271 | 0.0045968 | 0.087252 | 946 | 4 | −2.5598 | 0.97174 | 0.97169 | 1 | 16162 | 0 | −2.5598 |
| WDR45B | 4 | 0.001272 | 0.0046011 | 0.087252 | 947 | 4 | −1.1874 | 0.99873 | 0.99872 | 1 | 17331 | 0 | −1.1874 |
| MPDU1 | 4 | 0.0012762 | 0.0046105 | 0.087289 | 948 | 3 | −1.0452 | 0.82445 | 0.84242 | 1 | 13814 | 1 | −1.0452 |
| C2CD4D | 4 | 0.0012777 | 0.004617 | 0.087289 | 949 | 4 | −1.1394 | 0.99872 | 0.99872 | 1 | 17330 | 0 | −1.1394 |
| TIMM44 | 4 | 0.0012779 | 0.0046176 | 0.087289 | 950 | 4 | −1.5867 | 0.9949 | 0.995 | 1 | 16960 | 0 | −1.5867 |
| TMEM165 | 4 | 0.0012823 | 0.0046324 | 0.087478 | 951 | 4 | −1.0262 | 0.99872 | 0.99872 | 1 | 17328 | 0 | −1.0262 |
| MTA2 | 4 | 0.0012886 | 0.0046532 | 0.087779 | 952 | 4 | −1.4883 | 0.9949 | 0.995 | 1 | 16961 | 0 | −1.4883 |
| SNRPB2 | 4 | 0.0013017 | 0.0047026 | 0.088618 | 953 | 3 | −1.4407 | 0.95299 | 0.95284 | 1 | 15759 | 0 | −1.4407 |
| TSR3 | 3 | 0.0013037 | 0.0037297 | 0.076167 | 954 | 3 | −0.99075 | 0.9987 | 0.99873 | 1 | 17326 | 0 | −0.99075 |
| KIAAO087 | 4 | 0.0013058 | 0.0047152 | 0.088691 | 955 | 4 | −0.72242 | 0.99869 | 0.9987 | 1 | 17325 | 0 | −0.72242 |
| ASUN | 4 | 0.0013065 | 0.0047163 | 0.088691 | 956 | 4 | −1.1002 | 0.99869 | 0.9987 | 1 | 17324 | 0 | −1.1002 |
| GMPS | 4 | 0.0013094 | 0.0047256 | 0.088774 | 957 | 4 | −2.3668 | 0.98621 | 0.98627 | 1 | 16536 | 0 | −2.3668 |
| COQ6 | 4 | 0.0013116 | 0.0047361 | 0.088795 | 958 | 4 | −0.92872 | 0.99869 | 0.9987 | 1 | 17323 | 0 | −0.92872 |
| MORN3 | 4 | 0.001312 | 0.0047366 | 0.088795 | 959 | 4 | −1.2496 | 0.99869 | 0.9987 | 1 | 17322 | 0 | −1.2496 |
| GINS2 | 4 | 0.0013153 | 0.0047509 | 0.08897 | 960 | 3 | −3.6059 | 0.50069 | 0.65414 | 1 | 10880 | 1 | −3.6059 |
| CDIPT | 4 | 0.0013237 | 0.0047843 | 0.089429 | 961 | 4 | −1.9521 | 0.99868 | 0.99869 | 1 | 17321 | 0 | −1.9521 |
| ALG6 | 4 | 0.0013245 | 0.0047854 | 0.089429 | 962 | 4 | −1.0924 | 0.99868 | 0.99869 | 1 | 17320 | 0 | −1.0924 |
| C9orf78 | 4 | 0.0013258 | 0.0047904 | 0.089429 | 963 | 3 | −1.7411 | 0.87843 | 0.88076 | 1 | 14478 | 1 | −1.7411 |
| THOC1 | 4 | 0.0013281 | 0.0047964 | 0.089429 | 964 | 4 | −2.251 | 0.99564 | 0.99573 | 1 | 17016 | 0 | −2.251 |
| KIF14 | 4 | 0.0013308 | 0.0048008 | 0.089429 | 965 | 4 | −1.4373 | 0.99867 | 0.99868 | 1 | 17319 | 0 | −1.4373 |
| SRSF11 | 4 | 0.0013335 | 0.0048095 | 0.089429 | 966 | 4 | −1.9877 | 0.99867 | 0.99868 | 1 | 17317 | 0 | −1.9877 |
| MRPL51 | 4 | 0.0013344 | 0.0048139 | 0.089429 | 967 | 4 | −1.3671 | 0.99562 | 0.9957 | 1 | 17014 | 0 | −1.3671 |
| INTS10 | 4 | 0.001336 | 0.0048178 | 0.089429 | 968 | 3 | −1.2954 | 0.92809 | 0.92782 | 1 | 15263 | 0 | −1.2954 |
| ATP5J | 4 | 0.0013371 | 0.00482 | 0.089429 | 969 | 4 | −1.064 | 0.99866 | 0.99868 | 1 | 17316 | 0 | −1.064 |
| CTDSPL2 | 4 | 0.001343 | 0.0048419 | 0.089692 | 970 | 4 | −1.4276 | 0.99866 | 0.99867 | 1 | 17314 | 0 | −1.4276 |
| SRRT | 4 | 0.0013446 | 0.0048468 | 0.089692 | 971 | 4 | −0.77287 | 0.99866 | 0.99867 | 1 | 17313 | 0 | −0.77287 |
| NDUFC1 | 4 | 0.001345 | 0.004849 | 0.089692 | 972 | 4 | −1.1038 | 0.99866 | 0.99867 | 1 | 17312 | 0 | −1.1038 |
| FAM207A | 4 | 0.001347 | 0.0048551 | 0.089712 | 973 | 3 | −1.1906 | 0.6862 | 0.77412 | 1 | 12731 | 1 | −1.1906 |
| PALB2 | 4 | 0.0013557 | 0.0048913 | 0.090247 | 974 | 4 | −2.2926 | 0.99864 | 0.99866 | 1 | 17311 | 0 | −2.2926 |
| KIAA1432 | 4 | 0.0013565 | 0.004894 | 0.090247 | 975 | 4 | −2.7339 | 0.98139 | 0.98147 | 1 | 16396 | 0 | −2.7339 |
| YARS | 4 | 0.0013589 | 0.0049033 | 0.090326 | 976 | 4 | −3.7675 | 0.97689 | 0.97694 | 1 | 16281 | 0 | −3.7675 |
| TFB2M | 4 | 0.0013636 | 0.0049198 | 0.090445 | 977 | 4 | −1.3957 | 0.99778 | 0.99782 | 1 | 17200 | 0 | −1.3957 |
| CDC6 | 4 | 0.0013637 | 0.0049198 | 0.090445 | 978 | 4 | −1.5561 | 0.99864 | 0.99865 | 1 | 17309 | 0 | −1.5561 |
| CTC1 | 4 | 0.0013684 | 0.0049324 | 0.090584 | 979 | 4 | −1.4626 | 0.99566 | 0.99574 | 1 | 17017 | 0 | −1.4626 |
| PAK2 | 4 | 0.0013716 | 0.0049439 | 0.0906 | 980 | 4 | −1.3106 | 0.99819 | 0.99823 | 1 | 17240 | 0 | −1.3106 |
| AURKB | 4 | 0.0013721 | 0.0049445 | 0.0906 | 981 | 4 | −1.5155 | 0.99863 | 0.99864 | 1 | 17308 | 0 | −1.5155 |
| ERCC1 | 4 | 0.0013746 | 0.0049483 | 0.0906 | 982 | 4 | −1.3243 | 0.99863 | 0.99864 | 1 | 17307 | 0 | −1.3243 |
| GNL1 | 4 | 0.0013788 | 0.0049653 | 0.090819 | 983 | 3 | −1.7879 | 0.86866 | 0.87303 | 1 | 14322 | 1 | −1.7879 |
| LSM7 | 4 | 0.0013851 | 0.0049883 | 0.091014 | 984 | 4 | −1.6635 | 0.99861 | 0.99863 | 1 | 17306 | 0 | −1.6635 |
| HAUS5 | 4 | 0.0013869 | 0.0049905 | 0.091014 | 985 | 4 | −2.2545 | 0.98762 | 0.9877 | 1 | 16598 | 0 | −2.2545 |
| ZNHIT1 | 4 | 0.0013901 | 0.0050037 | 0.091094 | 986 | 3 | −1.5813 | 0.8038 | 0.83023 | 1 | 13607 | 1 | −1.5813 |
| DDX49 | 4 | 0.0013909 | 0.0050064 | 0.091094 | 987 | 3 | −4.8298 | 0.69285 | 0.7768 | 1 | 12774 | 1 | −4.8298 |
| XPO5 | 4 | 0.0013936 | 0.0050125 | 0.091094 | 988 | 4 | −1.4245 | 0.99861 | 0.99862 | 1 | 17304 | 0 | −1.4245 |
| ATG9A | 4 | 0.0013949 | 0.0050191 | 0.091094 | 989 | 4 | −2.4691 | 0.99684 | 0.99688 | 1 | 17100 | 0 | −2.4691 |
| PRPF4 | 4 | 0.0013956 | 0.0050207 | 0.091094 | 990 | 4 | −1.9069 | 0.9986 | 0.99862 | 1 | 17303 | 0 | −1.9069 |
| VPS72 | 4 | 0.0014014 | 0.0050465 | 0.09147 | 991 | 4 | −1.5519 | 0.99055 | 0.99064 | 1 | 16716 | 0 | −1.5519 |
| COX5A | 4 | 0.0014112 | 0.0050816 | 0.092014 | 992 | 4 | −1.1484 | 0.99859 | 0.9986 | 1 | 17302 | 0 | −1.1484 |
| PSMB3 | 4 | 0.0014145 | 0.0050947 | 0.09216 | 993 | 4 | −2.4788 | 0.99859 | 0.99859 | 1 | 17301 | 0 | −2.4788 |
| CCT5 | 4 | 0.0014331 | 0.005166 | 0.093356 | 994 | 4 | −1.7226 | 0.99857 | 0.99858 | 1 | 17300 | 0 | −1.7226 |
| RPL3 | 4 | 0.0014356 | 0.0051781 | 0.093466 | 995 | 4 | −2.7398 | 0.99856 | 0.99858 | 1 | 17298 | 0 | −2.7398 |
| ABCE1 | 4 | 0.0014369 | 0.0051825 | 0.093466 | 996 | 4 | −1.274 | 0.99856 | 0.99858 | 1 | 17296 | 0 | −1.274 |
| GCLC | 4 | 0.0014398 | 0.0051929 | 0.09356 | 997 | 4 | −1.1229 | 0.99856 | 0.99857 | 1 | 17295 | 0 | −1.1229 |
| OXSM | 4 | 0.0014435 | 0.0052055 | 0.093611 | 998 | 4 | −2.2388 | 0.99856 | 0.99857 | 1 | 17294 | 0 | −2.2388 |
| NASP | 4 | 0.001444 | 0.0052061 | 0.093611 | 999 | 3 | −1.6884 | 0.86644 | 0.87133 | 1 | 14296 | 1 | −1.6884 |
| MRPS18A | 4 | 0.0014469 | 0.0052181 | 0.093734 | 1000 | 4 | −0.91316 | 0.99855 | 0.99857 | 1 | 17293 | 0 | −0.91316 |
| ALG5 | 4 | 0.0014487 | 0.0052236 | 0.09374 | 1001 | 4 | −2.9408 | 0.99233 | 0.99245 | 1 | 16812 | 0 | −2.9408 |
| COX11 | 4 | 0.0014514 | 0.0052307 | 0.093774 | 1002 | 4 | −1.1542 | 0.9955 | 0.99559 | 1 | 17006 | 0 | −1.1542 |
| EIF4A1 | 4 | 0.0014581 | 0.0052549 | 0.094113 | 1003 | 4 | −2.4986 | 0.98771 | 0.98779 | 1 | 16602 | 0 | −2.4986 |
| DR1 | 4 | 0.0014616 | 0.0052697 | 0.094285 | 1004 | 3 | −1.7486 | 0.10991 | 0.24066 | 0.919533 | 4659 | 1 | −1.7486 |
| PPP2R5C | 4 | 0.0014658 | 0.0052883 | 0.094525 | 1005 | 4 | −1.4258 | 0.99853 | 0.99855 | 1 | 17292 | 0 | −1.4258 |
| MBTPS1 | 4 | 0.0014731 | 0.0053169 | 0.094851 | 1006 | 4 | −2.121 | 0.99841 | 0.99843 | 1 | 17272 | 0 | −2.121 |
| RANBP1 | 4 | 0.0014747 | 0.0053212 | 0.094851 | 1007 | 4 | −0.77028 | 0.99853 | 0.99855 | 1 | 17291 | 0 | −0.77028 |
| PLK4 | 4 | 0.0014772 | 0.0053284 | 0.094865 | 1008 | 3 | −2.2148 | 0.88759 | 0.88852 | 1 | 14604 | 1 | −2.2148 |
| NFS1 | 4 | 0.0014807 | 0.005341 | 0.094996 | 1009 | 3 | −1.8253 | 0.074256 | 0.18031 | 0.855267 | 3755 | 1 | −1.8253 |
| CDC23 | 4 | 0.001503 | 0.0054298 | 0.096481 | 1010 | 4 | −1.6665 | 0.9985 | 0.99852 | 1 | 17290 | 0 | −1.6665 |
| PDCD5 | 4 | 0.0015085 | 0.0054485 | 0.096615 | 1011 | 4 | −1.8365 | 0.96997 | 0.96988 | 1 | 16122 | 0 | −1.8365 |
| AHCYL1 | 4 | 0.0015099 | 0.0054529 | 0.096615 | 1012 | 4 | −1.1177 | 0.99849 | 0.99851 | 1 | 17289 | 0 | −1.1177 |
| MEF2BNB | 4 | 0.0015103 | 0.0054534 | 0.096615 | 1013 | 4 | −1.1474 | 0.97508 | 0.97507 | 1 | 16239 | 0 | −1.1474 |
| DIS3L | 4 | 0.0015256 | 0.0055148 | 0.097512 | 1014 | 3 | −1.1977 | 0.89414 | 0.89437 | 1 | 14709 | 1 | −1.1977 |
| IMP4 | 4 | 0.0015281 | 0.0055231 | 0.097562 | 1015 | 4 | −2.868 | 0.99847 | 0.9985 | 1 | 17288 | 0 | −2.868 |
| CPSF2 | 4 | 0.0015338 | 0.0055428 | 0.097815 | 1016 | 4 | −1.8048 | 0.99847 | 0.99849 | 1 | 17287 | 0 | −1.8048 |
| DNAJC11 | 3 | 0.0015355 | 0.0043686 | 0.084349 | 1017 | 3 | −1.5647 | 0.99846 | 0.9985 | 1 | 17286 | 0 | −1.5647 |
| UBC | 4 | 0.0015364 | 0.0055499 | 0.097827 | 1018 | 4 | −1.2803 | 0.99846 | 0.99849 | 1 | 17285 | 0 | −1.2803 |
| LAPTM4B | 4 | 0.0015373 | 0.0055543 | 0.097827 | 1019 | 4 | −0.66776 | 0.99846 | 0.99849 | 1 | 17284 | 0 | −0.66776 |
| HEXIM1 | 4 | 0.0015428 | 0.0055686 | 0.097849 | 1020 | 4 | −1.1189 | 0.99087 | 0.99099 | 1 | 16731 | 0 | −1.1189 |
| COMMD4 | 4 | 0.0015434 | 0.0055719 | 0.097849 | 1021 | 4 | −1.1795 | 0.99846 | 0.99848 | 1 | 17283 | 0 | −1.1795 |
| CYP1A2 | 4 | 0.0015478 | 0.00559 | 0.09986 | 1022 | 4 | −1.0458 | 0.99845 | 0.99848 | 1 | 17282 | 0 | −1.0458 |
| VPS25 | 4 | 0.0015483 | 0.0055905 | 0.097986 | 1023 | 4 | −2.4337 | 0.99845 | 0.99848 | 1 | 17281 | 0 | −2.4337 |
| RPS2 | 4 | 0.0015509 | 0.0055998 | 0.098054 | 1024 | 4 | −3.3063 | 0.99845 | 0.99848 | 1 | 17280 | 0 | −3.3063 |
| NSMCE2 | 4 | 0.0015531 | 0.0056053 | 0.098055 | 1025 | 4 | −0.99566 | 0.99845 | 0.99847 | 1 | 17279 | 0 | −0.99566 |
| RPS29 | 2 | 0.0015636 | 0.0030019 | 0.066009 | 1026 | 2 | −2.7724 | 0.99844 | 0.9984 | 1 | 17277 | 0 | −2.7724 |
| COPS4 | 4 | 0.0015696 | 0.005664 | 0.098986 | 1027 | 4 | −1.5676 | 0.99634 | 0.99641 | 1 | 17061 | 0 | −1.5676 |
| HAUS1 | 3 | 0.0015759 | 0.0044942 | 0.085947 | 1028 | 3 | −0.9363 | 0.99842 | 0.99846 | 1 | 17276 | 0 | −0.9363 |
| FAM73B | 4 | 0.0015771 | 0.005692 | 0.099379 | 1029 | 4 | −0.76677 | 0.99842 | 0.99845 | 1 | 17275 | 0 | −0.76677 |
| EXOC2 | 4 | 0.0015807 | 0.0057041 | 0.099493 | 1030 | 4 | −1.4309 | 0.99842 | 0.99844 | 1 | 17274 | 0 | −1.4309 |
| TYMS | 4 | 0.0015857 | 0.0057194 | 0.099665 | 1031 | 3 | −2.0669 | 0.83692 | 0.85036 | 1 | 13957 | 1 | −2.0669 |
| MESDC2 | 4 | 0.0015899 | 0.0057326 | 0.099798 | 1032 | 3 | −1.7594 | 0.94362 | 0.94348 | 1 | 15573 | 0 | −1.7594 |
| AARS | 4 | 0.0015933 | 0.0057441 | 0.099806 | 1033 | 4 | −2.8502 | 0.99841 | 0.99843 | 1 | 17273 | 0 | −2.8502 |
| MIS18A | 3 | 0.0017506 | 0.0049911 | 0.091014 | 1061 | 3 | −2.6081 | 0.95915 | 0.95941 | 1 | 15876 | 0 | −2.6081 |
| RPLP1 | 2 | 0.0017651 | 0.0034083 | 0.071805 | 1068 | 2 | −2.2282 | 0.99823 | 0.99818 | 1 | 17245 | 0 | −2.2282 |
| RPL7 | 3 | 0.0019351 | 0.0055055 | 0.097442 | 1092 | 3 | −2.6742 | 0.99806 | 0.99811 | 1 | 17227 | 0 | −2.6742 |
| GGNBP2 | 3 | 0.0020225 | 0.0057402 | 0.099806 | 1108 | 3 | −2.2296 | 0.99798 | 0.99803 | 1 | 17216 | 0 | −2.2296 |
| EIF1 | 2 | 0.0027533 | 0.0053223 | 0.094851 | 1217 | 2 | −1.918 | 0.99725 | 0.99716 | 1 | 17139 | 0 | −1.918 |
| UBE2N | 2 | 0.0028822 | 0.0055626 | 0.097849 | 1235 | 2 | −1.7603 | 0.99712 | 0.99703 | 1 | 17126 | 0 | −1.7603 |

H2030 MRTX SL Gene FDR 0.1

| | | | | | | | | | | | | | |
|---|---|---|---|---|---|---|---|---|---|---|---|---|---|
| STT3A | 4 | 1.45E−09 | 2.74E−07 | 0.000495 | 1 | 4 | −1.5021 | 1 | 1 | 1 | 18046 | 0 | −1.5021 |
| SHOC2 | 4 | 1.79E−09 | 2.74E−07 | 0.000495 | 2 | 4 | −0.67906 | 1 | 1 | 1 | 18052 | 0 | −0.67906 |
| PIK3CB | 4 | 1.02E−08 | 2.74E−07 | 0.000495 | 3 | 4 | −0.67685 | 1 | 1 | 1 | 18051 | 0 | −0.67685 |
| ZFY | 4 | 1.04E−08 | 2.74E−07 | 0.000495 | 4 | 4 | −0.9875 | 0.99999 | 0.99999 | 1 | 18021 | 0 | −0.9875 |
| PTBP1 | 4 | 1.10E−08 | 2.74E−07 | 0.000495 | 5 | 4 | −1.2553 | 1 | 1 | 1 | 18050 | 0 | −1.2553 |
| DOT1L | 4 | 1.73E−08 | 2.74E−07 | 0.000495 | 6 | 4 | −0.9841 | 1 | 1 | 1 | 18049 | 0 | −0.9841 |
| UGP2 | 4 | 2.56E−08 | 2.74E−07 | 0.000495 | 7 | 4 | −1.0573 | 0.99996 | 0.99996 | 1 | 18002 | 0 | −1.0573 |
| PDPK1 | 4 | 3.37E−08 | 2.74E−07 | 0.000495 | 8 | 4 | −1.0696 | 1 | 1 | 1 | 18048 | 0 | −1.0696 |
| TEAD1 | 4 | 4.22E−08 | 2.74E−07 | 0.000495 | 9 | 4 | −1.0036 | 1 | 1 | 1 | 18047 | 0 | −1.0036 |
| SLC31A1 | 4 | 9.14E−08 | 2.74E−07 | 0.000495 | 10 | 4 | −0.78987 | 1 | 1 | 1 | 18045 | 0 | −0.78987 |
| PPTC7 | 4 | 1.29E−07 | 8.23E−07 | 0.00099 | 11 | 4 | −0.83223 | 1 | 1 | 1 | 18044 | 0 | −0.83223 |
| ALG8 | 4 | 1.37E−07 | 8.23E−07 | 0.00099 | 12 | 4 | −0.99803 | 1 | 1 | 1 | 18027 | 0 | −0.99803 |
| RTCB | 4 | 1.43E−07 | 8.23E−07 | 0.00099 | 13 | 4 | −1.3795 | 1 | 1 | 1 | 18043 | 0 | −1.3795 |
| MPI | 4 | 1.64E−07 | 8.23E−07 | 0.00099 | 14 | 4 | −0.82805 | 1 | 1 | 1 | 18042 | 0 | −0.82805 |
| EXT2 | 4 | 1.81E−07 | 8.23E−07 | 0.00099 | 15 | 4 | −0.83443 | 0.99873 | 0.99873 | 1 | 17867 | 0 | −0.83443 |
| STT3B | 4 | 2.42E−07 | 1.37E−06 | 0.001238 | 16 | 3 | −0.79363 | 0.76664 | 0.76646 | 1 | 12910 | 0 | −0.79363 |
| MMS19 | 4 | 3.04E−07 | 1.37E−06 | 0.001238 | 17 | 4 | −0.65606 | 1 | 1 | 1 | 18041 | 0 | −0.65606 |
| NDST1 | 4 | 3.87E−07 | 1.37E−06 | 0.001238 | 18 | 4 | −0.68696 | 1 | 1 | 1 | 18040 | 0 | −0.68696 |
| ELP5 | 4 | 3.87E−07 | 1.37E−06 | 0.001238 | 19 | 4 | −1.8436 | 1 | 1 | 1 | 18035 | 0 | −1.8436 |
| RLF | 4 | 4.57E−07 | 1.37E−06 | 0.00238 | 20 | 4 | −0.60154 | 1 | 1 | 1 | 18039 | 0 | −0.60154 |
| TMEM165 | 4 | 7.17E−07 | 1.92E−06 | 0.001238 | 21 | 3 | −0.84337 | 0.97613 | 0.9764 | 1 | 17100 | 0 | −0.84337 |
| YAP1 | 4 | 7.81E−07 | 1.92E−06 | 0.001238 | 22 | 4 | −0.86906 | 1 | 1 | 1 | 18033 | 0 | −0.86906 |
| SLC39A9 | 4 | 8.15E−07 | 1.92E−06 | 0.001238 | 23 | 4 | −0.86457 | 0.99957 | 0.99958 | 1 | 17932 | 0 | −0.86457 |
| TEN1 | 4 | 8.24E−07 | 1.92E−06 | 0.001238 | 24 | 4 | −1.0114 | 1 | 1 | 1 | 18038 | 0 | −1.0114 |
| FBXL4 | 4 | 8.49E−07 | 1.92E−06 | 0.00238 | 25 | 4 | −0.85328 | 1 | 1 | 1 | 18037 | 0 | −0.85328 |
| EXTL3 | 4 | 8.83E−07 | 1.92E−06 | 0.001238 | 26 | 4 | −0.74608 | 1 | 1 | 1 | 18036 | 0 | −0.74608 |
| LEMD2 | 4 | 8.88E−07 | 1.92E−06 | 0.001238 | 27 | 3 | −0.99435 | 0.99351 | 0.99357 | 1 | 17645 | 0 | −0.99435 |
| AKT1 | 4 | 9.00E−07 | 1.92E−06 | 0.00238 | 28 | 4 | −0.82176 | 0.99988 | 0.99988 | 1 | 17973 | 0 | −0.82176 |
| EXT1 | 4 | 1.19E−06 | 3.57E−06 | 0.002011 | 29 | 4 | −0.86271 | 0.99999 | 0.99999 | 1 | 18023 | 0 | −0.86271 |
| IKBKAP | 4 | 1.20E−06 | 3.57E−06 | 0.002011 | 30 | 4 | −0.88692 | 0.99954 | 0.99955 | 1 | 17931 | 0 | −0.88692 |
| PTPMT1 | 4 | 1.21E−06 | 3.57E−06 | 0.002011 | 31 | 4 | −0.87539 | 1 | 1 | 1 | 18034 | 0 | −0.87539 |
| HS2ST1 | 4 | 1.26E−06 | 3.57E−06 | 0.002011 | 32 | 3 | −0.71175 | 0.8586 | 0.85871 | 1 | 14595 | 0 | −0.71175 |
| B3GNT2 | 4 | 1.67E−06 | 4.66E−06 | 0.00255 | 33 | 4 | −0.68468 | 0.99996 | 0.99996 | 1 | 18003 | 0 | −0.68468 |
| SRP68 | 4 | 1.95E−06 | 5.76E−06 | 0.00297 | 34 | 3 | −1.0876 | 0.84039 | 0.84046 | 1 | 14254 | 0 | −1.0876 |
| RPN1 | 4 | 1.97E−06 | 5.76E−06 | 0.00297 | 35 | 4 | −1.0111 | 1 | 1 | 1 | 18032 | 0 | −1.0111 |
| RAB10 | 4 | 2.00E−06 | 6.31E−06 | 0.003163 | 36 | 4 | −0.86859 | 1 | 1 | 1 | 18031 | 0 | −0.86859 |
| ELP3 | 4 | 2.50E−06 | 7.40E−06 | 0.003517 | 37 | 4 | −1.1683 | 1 | 1 | 1 | 18030 | 0 | −1.1683 |
| DNAJB11 | 4 | 2.59E−06 | 7.40E−06 | 0.003517 | 38 | 4 | −0.51816 | 1 | 1 | 1 | 18029 | 0 | −0.51816 |
| MANF | 4 | 2.63E−06 | 7.95E−06 | 0.003681 | 39 | 3 | −0.64604 | 0.33104 | 0.45462 | 1 | 7522 | 1 | −0.64604 |
| B3GNT5 | 4 | 2.84E−06 | 9.60E−06 | 0.004332 | 40 | 4 | −0.60003 | 1 | 1 | 1 | 18028 | 0 | −0.60003 |
| RNF145 | 4 | 3.23E−06 | 1.12E−05 | 0.00495 | 41 | 4 | −0.56323 | 1 | 1 | 1 | 18026 | 0 | −0.56323 |
| FKBPL | 4 | 3.33E−06 | 1.18E−05 | 0.00495 | 42 | 4 | −0.53495 | 1 | 1 | 1 | 18025 | 0 | −0.53495 |
| TRIT1 | 4 | 3.58E−06 | 1.18E−05 | 0.00495 | 43 | 4 | −1.614 | 1 | 1 | 1 | 18024 | 0 | −1.614 |
| MIS18A | 4 | 3.94E−06 | 1.40E−05 | 0.005738 | 44 | 3 | −0.73699 | 0.99224 | 0.99228 | 1 | 17592 | 0 | −0.73699 |
| THOC6 | 4 | 4.09E−06 | 1.45E−05 | 0.005831 | 45 | 4 | −0.75645 | 0.99999 | 0.99998 | 1 | 18017 | 0 | −0.75645 |
| GRPEL1 | 4 | 4.32E−06 | 1.56E−05 | 0.006134 | 46 | 4 | −1.7109 | 0.99929 | 0.99931 | 1 | 17906 | 0 | −1.7109 |
| ILF3 | 4 | 4.41E−06 | 1.62E−05 | 0.006214 | 47 | 4 | −1.0602 | 0.99935 | 0.99937 | 1 | 17915 | 0 | −1.0602 |
| EIF3H | 4 | 4.48E−06 | 1.67E−05 | 0.006291 | 48 | 3 | −0.72507 | 0.98064 | 0.9808 | 1 | 17230 | 0 | −0.72507 |
| GEMIN7 | 4 | 5.83E−06 | 2.17E−05 | 0.007981 | 49 | 4 | −1.0867 | 0.99999 | 0.99999 | 1 | 18022 | 0 | −1.0867 |
| MOGS | 4 | 6.99E−06 | 2.71E−05 | 0.009802 | 50 | 4 | −0.6879 | 0.99787 | 0.99788 | 1 | 17825 | 0 | −0.6879 |
| MLST8 | 4 | 7.56E−06 | 2.99E−05 | 0.01058 | 51 | 4 | −0.82634 | 0.99983 | 0.99984 | 1 | 17967 | 0 | −0.82634 |
| GLYR1 | 4 | 7.93E−06 | 3.21E−05 | 0.011139 | 52 | 4 | −0.56313 | 0.99878 | 0.99878 | 1 | 17874 | 0 | −0.56313 |
| CTU2 | 4 | 8.73E−06 | 3.54E−05 | 0.012049 | 53 | 3 | −1.0148 | 0.76417 | 0.76393 | 1 | 12865 | 0 | −1.0148 |
| ADNP | 4 | 9.14E−06 | 3.70E−05 | 0.012331 | 54 | 4 | −0.66546 | 0.99741 | 0.99742 | 1 | 17802 | 0 | −0.66546 |
| WASF2 | 4 | 9.19E−06 | 3.76E−05 | 0.012331 | 55 | 4 | −0.684 | 0.99999 | 0.99999 | 1 | 18020 | 0 | −0.684 |
| COMMD2 | 4 | 1.13E−05 | 4.42E−05 | 0.014233 | 56 | 4 | −0.58799 | 0.99991 | 0.99991 | 1 | 17982 | 0 | −0.58799 |
| AHCYL1 | 4 | 1.24E−05 | 5.18E−05 | 0.01548 | 57 | 3 | −0.65154 | 0.95965 | 0.9598 | 1 | 16701 | 0 | −0.65154 |
| DEXI | 4 | 1.26E−05 | 5.24E−05 | 0.01548 | 58 | 4 | −0.42268 | 0.99999 | 0.99998 | 1 | 18019 | 0 | −0.42268 |
| COL4A3BP | 4 | 1.26E−05 | 5.24E−05 | 0.01548 | 59 | 4 | −0.4812 | 0.99999 | 0.99998 | 1 | 18018 | 0 | −0.4812 |
| GNG12 | 4 | 1.26E−05 | 5.24E−05 | 0.01548 | 60 | 4 | −0.50734 | 0.9979 | 0.9979 | 1 | 17828 | 0 | −0.50734 |
| ALG5 | 4 | 1.30E−05 | 5.35E−05 | 0.01548 | 61 | 4 | −0.9229 | 0.99605 | 0.99606 | 1 | 17747 | 0 | −0.9229 |
| SPTLC1 | 4 | 1.31E−05 | 5.40E−05 | 0.01548 | 62 | 3 | −0.84186 | 0.9482 | 0.94837 | 1 | 16434 | 0 | −0.84186 |
| DOCK5 | 4 | 1.44E−05 | 5.73E−05 | 0.016166 | 63 | 4 | −0.49758 | 0.99999 | 0.99998 | 1 | 18016 | 0 | −0.49758 |
| GMPPB | 3 | 1.47E−05 | 5.07E−05 | 0.01548 | 64 | 3 | −1.4316 | 0.99999 | 0.99998 | 1 | 18015 | 0 | −1.4316 |
| B3GAT3 | 4 | 1.60E−05 | 6.31E−05 | 0.017327 | 65 | 3 | −0.84267 | 0.5951 | 0.61463 | 1 | 10232 | 1 | −0.84267 |
| GET4 | 4 | 1.60E−05 | 6.33E−05 | 0.017327 | 66 | 4 | −0.62429 | 0.99998 | 0.99998 | 1 | 18014 | 0 | −0.62429 |
| OPA1 | 4 | 1.80E−05 | 7.32E−05 | 0.019448 | 67 | 4 | −0.85344 | 0.99998 | 0.99998 | 1 | 18013 | 0 | −0.85344 |
| HNF1B | 4 | 1.86E−05 | 7.54E−05 | 0.019448 | 68 | 3 | −0.83522 | 0.92438 | 0.92467 | 1 | 15931 | 0 | −0.83522 |
| URM1 | 4 | 1.88E−05 | 7.54E−05 | 0.019448 | 69 | 3 | −0.95367 | 0.72731 | 0.72714 | 1 | 12209 | 1 | −0.95367 |
| SMARCA2 | 4 | 1.90E−05 | 7.65E−05 | 0.019453 | 70 | 3 | −0.65632 | 0.82637 | 0.8265 | 1 | 13996 | 0 | −0.65632 |
| TM9SF2 | 4 | 2.02E−05 | 8.36E−05 | 0.020684 | 71 | 3 | −0.53583 | 0.88708 | 0.88726 | 1 | 15137 | 0 | −0.53583 |
| ELP2 | 4 | 2.03E−05 | 8.36E−05 | 0.020684 | 72 | 4 | −0.9854 | 0.99827 | 0.99828 | 1 | 17846 | 0 | −0.9854 |
| ACTR2 | 4 | 2.27E−05 | 9.41E−05 | 0.022946 | 73 | 4 | −1.39 | 0.99998 | 0.99998 | 1 | 18012 | 0 | −1.39 |
| WDR47 | 3 | 2.28E−05 | 7.49E−05 | 0.019448 | 74 | 3 | −0.49475 | 0.99998 | 0.99998 | 1 | 18011 | 0 | −0.49475 |
| MAPK1 | 4 | 2.33E−05 | 9.68E−05 | 0.0233 | 75 | 4 | −0.46126 | 0.99998 | 0.99998 | 1 | 18010 | 0 | −0.46126 |
| MTX1 | 4 | 2.47E−05 | 0.00010119 | 0.024036 | 76 | 4 | −0.49681 | 0.99998 | 0.99998 | 1 | 18009 | 0 | −0.49681 |
| PKN2 | 4 | 2.58E−05 | 0.00010394 | 0.024367 | 77 | 4 | −0.80445 | 0.99995 | 0.99994 | 1 | 17998 | 0 | −0.80445 |
| KIAA0100 | 4 | 2.64E−05 | 0.00010668 | 0.024376 | 78 | 4 | −0.62443 | 0.99997 | 0.99997 | 1 | 18008 | 0 | −0.62443 |
| NCAPD2 | 4 | 3.00E−05 | 0.0001171 | 0.02602 | 79 | 3 | −0.95793 | 0.9819 | 0.98202 | 1 | 17273 | 0 | −0.95793 |
| IPO11 | 4 | 3.01E−05 | 0.00011765 | 0.02602 | 80 | 4 | −0.76866 | 0.99997 | 0.99997 | 1 | 18007 | 0 | −0.76866 |
| CDIPT | 4 | 3.03E−05 | 0.0001182 | 0.02602 | 81 | 3 | −1.0714 | 0.88435 | 0.88454 | 1 | 15067 | 0 | −1.0714 |
| PPP4R1 | 4 | 3.20E−05 | 0.00012341 | 0.02684 | 82 | 4 | −0.52026 | 0.99991 | 0.99992 | 1 | 17984 | 0 | −0.52026 |
| UXS1 | 3 | 3.22E−05 | 0.00010558 | 0.024376 | 83 | 3 | −0.6615 | 0.99705 | 0.99708 | 1 | 17783 | 0 | −0.6615 |
| WAPAL | 4 | 3.39E−05 | 0.00013081 | 0.028112 | 84 | 4 | −0.64674 | 0.99997 | 0.99996 | 1 | 18006 | 0 | −0.64674 |
| EXOSC2 | 4 | 3.70E−05 | 0.00014233 | 0.029874 | 85 | 4 | −1.0085 | 0.99996 | 0.99996 | 1 | 18005 | 0 | −1.0085 |
| PDCL | 4 | 3.76E−05 | 0.00014343 | 0.029874 | 86 | 3 | −0.71675 | 0.99407 | 0.99413 | 1 | 17667 | 0 | −0.71675 |
| SP3 | 4 | 3.79E−05 | 0.00014397 | 0.029874 | 87 | 3 | −0.83766 | 0.98851 | 0.98857 | 1 | 17482 | 0 | −0.83766 |
| CCDC22 | 4 | 3.88E−05 | 0.00014891 | 0.029975 | 88 | 3 | −0.50405 | 0.55132 | 0.58342 | 1 | 9689 | 1 | −0.50405 |
| WWTR1 | 4 | 3.88E−05 | 0.00014946 | 0.029975 | 89 | 3 | −0.76471 | 0.95953 | 0.95969 | 1 | 16698 | 0 | −0.76471 |
| SEPSECS | 4 | 3.93E−05 | 0.0001511 | 0.029975 | 90 | 3 | −0.87034 | 0.6492 | 0.65687 | 1 | 10956 | 1 | −0.87034 |
| MESDC2 | 4 | 3.93E−05 | 0.0001511 | 0.029975 | 91 | 4 | −0.63177 | 0.99996 | 0.99996 | 1 | 18004 | 0 | −0.63177 |
| GANAB | 4 | 4.14E−05 | 0.00016207 | 0.031802 | 92 | 4 | −0.70423 | 0.99931 | 0.99933 | 1 | 17908 | 0 | −0.70423 |
| KTI12 | 4 | 4.32E−05 | 0.0001692 | 0.0326 | 93 | 4 | −1.0089 | 0.99915 | 0.99915 | 1 | 17898 | 0 | −1.0089 |
| MAPKAP1 | 4 | 4.37E−05 | 0.00016975 | 0.0326 | 94 | 4 | −0.80569 | 0.99918 | 0.99918 | 1 | 17900 | 0 | −0.80569 |
| EIF3M | 4 | 4.49E−05 | 0.00017304 | 0.032642 | 95 | 3 | −0.96693 | 0.92451 | 0.9248 | 1 | 15935 | 0 | −0.96693 |
| CNOT11 | 4 | 4.51E−05 | 0.00017359 | 0.032642 | 96 | 3 | −0.85121 | 0.99445 | 0.99451 | 1 | 17684 | 0 | −0.85121 |
| ERBB3 | 4 | 4.74E−05 | 0.00018511 | 0.034449 | 97 | 3 | −0.59828 | 0.36677 | 0.47314 | 1 | 7817 | 1 | −0.59828 |
| TRH | 4 | 4.84E−05 | 0.0001873 | 0.034502 | 98 | 4 | −0.46312 | 0.99995 | 0.99995 | 1 | 18001 | 0 | −0.46312 |
| TAF4 | 4 | 5.01E−05 | 0.00019553 | 0.035594 | 99 | 4 | −0.29155 | 0.99995 | 0.99995 | 1 | 18000 | 0 | −0.29155 |
| RAF1 | 4 | 5.03E−05 | 0.00019718 | 0.035594 | 100 | 4 | −0.63927 | 0.99991 | 0.99991 | 1 | 17981 | 0 | −0.63927 |
| C1orf27 | 4 | 5.15E−05 | 0.00020156 | 0.035964 | 101 | 3 | −0.95638 | 0.99147 | 0.99152 | 1 | 17573 | 0 | −0.95638 |
| PSMD6 | 4 | 5.20E−05 | 0.00020321 | 0.035964 | 102 | 3 | −0.73616 | 0.85195 | 0.85204 | 1 | 14469 | 0 | −0.73616 |
| CRKL | 4 | 5.35E−05 | 0.00020979 | 0.036768 | 103 | 4 | −0.58418 | 0.99995 | 0.99994 | 1 | 17999 | 0 | −0.58418 |
| EEFSEC | 4 | 5.70E−05 | 0.0002246 | 0.038985 | 104 | 4 | −0.50223 | 0.99994 | 0.99994 | 1 | 17997 | 0 | −0.50223 |
| SNX27 | 4 | 5.76E−05 | 0.00022789 | 0.038997 | 105 | 4 | −0.37485 | 0.99994 | 0.99994 | 1 | 17996 | 0 | −0.37485 |
| CCND3 | 4 | 5.78E−05 | 0.00022899 | 0.038997 | 106 | 4 | −0.64979 | 0.99994 | 0.99994 | 1 | 17995 | 0 | −0.64979 |
| SLC35B2 | 4 | 5.90E−05 | 0.00023337 | 0.039373 | 107 | 3 | −0.67267 | 0.9889 | 0.98895 | 1 | 17495 | 0 | −0.67267 |
| C17orf70 | 4 | 6.08E−05 | 0.00023941 | 0.040017 | 108 | 4 | −0.7981 | 0.9998 | 0.9998 | 1 | 17960 | 0 | −0.7981 |
| ARPC3 | 4 | 6.26E−05 | 0.00024928 | 0.041226 | 109 | 4 | −0.57241 | 0.99994 | 0.99994 | 1 | 17994 | 0 | −0.57241 |
| TBP | 4 | 6.38E−05 | 0.00025312 | 0.041226 | 110 | 3 | −0.74111 | 0.19373 | 0.30548 | 1 | 5130 | 1 | −0.74111 |
| VPS29 | 4 | 6.46E−05 | 0.00025806 | 0.041226 | 111 | 4 | −0.75186 | 0.99992 | 0.99992 | 1 | 17987 | 0 | −0.75186 |
| SYVN1 | 4 | 6.51E−05 | 0.0002597 | 0.041226 | 112 | 4 | −0.69061 | 0.99625 | 0.99629 | 1 | 17751 | 0 | −0.69061 |
| PTDSS1 | 4 | 6.60E−05 | 0.00026189 | 0.041226 | 113 | 4 | −0.6689 | 0.99958 | 0.99958 | 1 | 17933 | 0 | −0.6689 |
| CIRH1A | 4 | 6.69E−05 | 0.00026381 | 0.041226 | 114 | 4 | −1.0101 | 0.99993 | 0.99993 | 1 | 17993 | 0 | −1.0101 |
| PARD6B | 4 | 6.72E−05 | 0.00026519 | 0.041226 | 115 | 4 | −0.54655 | 0.99991 | 0.99991 | 1 | 17983 | 0 | −0.54655 |
| ZW10 | 4 | 6.74E−05 | 0.00026628 | 0.041226 | 116 | 3 | −0.54528 | 0.83099 | 0.83117 | 1 | 14078 | 0 | −0.54528 |
| PRPF3 | 4 | 6.80E−05 | 0.00026902 | 0.041226 | 117 | 4 | −0.44429 | 0.99993 | 0.99993 | 1 | 17992 | 0 | −0.44429 |
| CENPN | 4 | 6.83E−05 | 0.00026957 | 0.041226 | 118 | 2 | −0.56287 | 0.72596 | 0.72585 | 1 | 12183 | 1 | −0.56287 |
| RIC8A | 4 | 6.88E−05 | 0.00027177 | 0.041226 | 119 | 4 | −0.39673 | 0.99993 | 0.99993 | 1 | 17991 | 0 | −0.39673 |
| IPO9 | 4 | 7.29E−05 | 0.00028109 | 0.042181 | 120 | 4 | −0.58191 | 0.99502 | 0.99506 | 1 | 17700 | 0 | −0.58191 |
| RAPH1 | 4 | 7.32E−05 | 0.00028274 | 0.042181 | 121 | 4 | −0.43402 | 0.99993 | 0.99993 | 1 | 17990 | 0 | −0.43402 |
| ACTR3 | 4 | 7.48E−05 | 0.00028932 | 0.04281 | 122 | 4 | −0.48897 | 0.99993 | 0.99992 | 1 | 17989 | 0 | −0.48897 |
| NFAT5 | 4 | 7.64E−05 | 0.0002948 | 0.043267 | 123 | 3 | −0.56522 | 0.97645 | 0.97672 | 1 | 17107 | 0 | −0.56522 |
| UFSP2 | 4 | 7.82E−05 | 0.00030193 | 0.043956 | 124 | 4 | −0.63212 | 0.99992 | 0.99992 | 1 | 17988 | 0 | −0.63212 |
| TYRP1 | 4 | 8.48E−05 | 0.00032607 | 0.046951 | 125 | 4 | −0.36935 | 0.99992 | 0.99992 | 1 | 17986 | 0 | −0.36935 |
| RABIF | 4 | 8.51E−05 | 0.00032771 | 0.046951 | 126 | 4 | −0.52176 | 0.99991 | 0.99992 | 1 | 17985 | 0 | −0.52176 |
| PIGL | 4 | 9.19E−05 | 0.00035075 | 0.049856 | 127 | 3 | −0.62308 | 0.87443 | 0.87459 | 1 | 14888 | 0 | −0.62308 |
| SEC63 | 4 | 9.43E−05 | 0.00036117 | 0.050936 | 128 | 4 | −0.73754 | 0.99876 | 0.99875 | 1 | 17871 | 0 | −0.73754 |
| MSTO1 | 4 | 9.56E−05 | 0.0003683 | 0.051539 | 129 | 4 | −0.86869 | 0.99824 | 0.99825 | 1 | 17844 | 0 | −0.86869 |
| ILF2 | 4 | 9.69E−05 | 0.00037488 | 0.052056 | 130 | 2 | −0.40314 | 0.42355 | 0.5043 | 1 | 8334 | 1 | −0.40314 |
| ANKRD49 | 4 | 9.94E−05 | 0.00038201 | 0.052642 | 131 | 4 | −0.48304 | 0.9999 | 0.9999 | 1 | 17980 | 0 | −0.48304 |
| BRK1 | 4 | 0.00010244 | 0.00039133 | 0.053488 | 132 | 4 | −0.71348 | 0.99521 | 0.99525 | 1 | 17707 | 0 | −0.71348 |
| PGD | 4 | 0.00010406 | 0.00039408 | 0.053488 | 133 | 4 | −0.69848 | 0.9999 | 0.9999 | 1 | 17979 | 0 | −0.69848 |
| HDAC3 | 4 | 0.00010595 | 0.0004023 | 0.054197 | 134 | 4 | −1.0691 | 0.99977 | 0.99977 | 1 | 17957 | 0 | −1.0691 |
| ALG9 | 4 | 0.00010712 | 0.00040724 | 0.054455 | 135 | 4 | −0.5445 | 0.99936 | 0.99938 | 1 | 17916 | 0 | −0.5445 |
| ACSL3 | 4 | 0.00010988 | 0.00041492 | 0.055074 | 136 | 4 | −0.59502 | 0.99989 | 0.99989 | 1 | 17978 | 0 | −0.59502 |
| SLMAP | 4 | 0.00011332 | 0.00042918 | 0.056379 | 137 | 4 | −0.50154 | 0.99989 | 0.99989 | 1 | 17977 | 0 | −0.50154 |
| NAA40 | 4 | 0.00011413 | 0.00043192 | 0.056379 | 138 | 4 | −0.42433 | 0.99989 | 0.99989 | 1 | 17976 | 0 | −0.42433 |
| RIMS2 | 4 | 0.00011475 | 0.00043411 | 0.056379 | 139 | 4 | −0.34239 | 0.99989 | 0.99989 | 1 | 17975 | 0 | −0.34239 |
| DDX6 | 4 | 0.00012133 | 0.00046099 | 0.059441 | 140 | 3 | −1.0329 | 0.92792 | 0.92822 | 1 | 16008 | 0 | −1.0329 |
| TECR | 4 | 0.00012317 | 0.00046702 | 0.059792 | 141 | 4 | −0.50148 | 0.99988 | 0.99988 | 1 | 17974 | 0 | −0.50148 |
| VRK1 | 4 | 0.00012957 | 0.0004917 | 0.062509 | 142 | 4 | −0.47704 | 0.99987 | 0.99988 | 1 | 17972 | 0 | −0.47704 |
| PIGM | 4 | 0.00013752 | 0.00052845 | 0.066247 | 143 | 4 | −0.51491 | 0.99979 | 0.9998 | 1 | 17958 | 0 | −0.51491 |
| PAK1IP1 | 4 | 0.00014 | 0.00053668 | 0.066815 | 144 | 3 | −0.77293 | 0.98853 | 0.9886 | 1 | 17486 | 0 | −0.77293 |
| PRKCSH | 4 | 0.0001451 | 0.00055807 | 0.069002 | 145 | 3 | −0.48155 | 0.99469 | 0.99474 | 1 | 17692 | 0 | −0.48155 |
| EXOC5 | 4 | 0.00014947 | 0.00057233 | 0.070284 | 146 | 3 | −0.6309 | 0.48435 | 0.54019 | 1 | 8944 | 1 | −0.6309 |
| DNM1L | 4 | 0.00015326 | 0.0005833 | 0.071147 | 147 | 4 | −0.53441 | 0.99985 | 0.99985 | 1 | 17971 | 0 | −0.53441 |
| ELMO2 | 4 | 0.00015839 | 0.00059975 | 0.072663 | 148 | 3 | −1.0327 | 0.98245 | 0.98256 | 1 | 17288 | 0 | −1.0327 |
| ITGB5 | 4 | 0.0001646 | 0.00062059 | 0.074686 | 149 | 4 | −0.68627 | 0.99984 | 0.99984 | 1 | 17970 | 0 | −0.68627 |
| SCAF4 | 4 | 0.00016617 | 0.00062498 | 0.074716 | 150 | 4 | −0.30325 | 0.99983 | 0.99984 | 1 | 17969 | 0 | −0.30325 |
| RAVER2 | 4 | 0.00016958 | 0.00063485 | 0.075397 | 151 | 4 | −0.39312 | 0.99983 | 0.99984 | 1 | 17968 | 0 | −0.39312 |
| COL8A2 | 4 | 0.00017339 | 0.00064911 | 0.076587 | 152 | 4 | −0.40642 | 0.99983 | 0.99983 | 1 | 17966 | 0 | −0.40642 |
| EIF2B5 | 4 | 0.00017528 | 0.00065679 | 0.07699 | 153 | 4 | −0.74008 | 0.99982 | 0.99983 | 1 | 17965 | 0 | −0.74008 |
| ICMT | 4 | 0.00017733 | 0.00066338 | 0.077018 | 154 | 4 | −0.67628 | 0.99588 | 0.99589 | 1 | 17736 | 0 | −0.67628 |
| SRP54 | 4 | 0.00017851 | 0.00066557 | 0.077018 | 155 | 3 | −0.89595 | 0.75056 | 0.75039 | 1 | 12624 | 0 | −0.89595 |
| GABPB1 | 4 | 0.00018076 | 0.00067051 | 0.077095 | 156 | 4 | −0.47102 | 0.99982 | 0.99983 | 1 | 17964 | 0 | −0.47102 |
| NCKAP1 | 4 | 0.00018547 | 0.00068422 | 0.078174 | 157 | 4 | −0.54501 | 0.99981 | 0.99982 | 1 | 17963 | 0 | −0.54501 |
| TFAP4 | 4 | 0.00018788 | 0.00069135 | 0.078492 | 158 | 3 | −0.6127 | 0.85775 | 0.85788 | 1 | 14581 | 0 | −0.6127 |
| RIOK2 | 4 | 0.00019091 | 0.00070286 | 0.079055 | 159 | 4 | −0.5418 | 0.99981 | 0.99981 | 1 | 17962 | 0 | −0.5418 |
| FAM3A | 4 | 0.00019321 | 0.0007089 | 0.079055 | 160 | 3 | −0.49581 | 0.9931 | 0.99314 | 1 | 17628 | 0 | −0.49581 |
| PKM | 4 | 0.00019356 | 0.00070945 | 0.079055 | 161 | 3 | −0.46313 | 0.95464 | 0.95476 | 1 | 16566 | 0 | −0.46313 |
| NCOA4 | 4 | 0.00020016 | 0.00073358 | 0.081243 | 162 | 4 | −0.32493 | 0.9998 | 0.9998 | 1 | 17961 | 0 | −0.32493 |
| FERMT2 | 4 | 0.00020139 | 0.00073961 | 0.081411 | 163 | 3 | −0.69451 | 0.43931 | 0.51321 | 1 | 8486 | 1 | −0.69451 |
| EIF3F | 4 | 0.00020287 | 0.00074619 | 0.081638 | 164 | 4 | −0.94943 | 0.99555 | 0.99556 | 1 | 17722 | 0 | −0.94943 |
| PGM3 | 4 | 0.00020524 | 0.00075442 | 0.082041 | 165 | 4 | −0.39823 | 0.99979 | 0.9998 | 1 | 17959 | 0 | −0.39823 |
| DOCK7 | 4 | 0.00020989 | 0.00077033 | 0.083269 | 166 | 3 | −0.507 | 0.97753 | 0.97781 | 1 | 17135 | 0 | −0.507 |
| KARS | 4 | 0.00021364 | 0.00078404 | 0.084247 | 167 | 3 | −1.2036 | 0.9846 | 0.98467 | 1 | 17351 | 0 | −1.2036 |
| RHOV | 4 | 0.00022515 | 0.00082133 | 0.087216 | 168 | 4 | −0.43638 | 0.99973 | 0.99973 | 1 | 17952 | 0 | −0.43638 |
| CENPO | 4 | 0.00022809 | 0.0008334 | 0.08798 | 169 | 3 | −0.61097 | 0.98821 | 0.98828 | 1 | 17464 | 0 | −0.61097 |
| B4GALT3 | 4 | 0.00023308 | 0.00084766 | 0.088965 | 170 | 4 | −0.58004 | 0.99977 | 0.99977 | 1 | 17956 | 0 | −0.58004 |
| MCL1 | 4 | 0.00023745 | 0.00086082 | 0.089824 | 171 | 3 | −0.45828 | 0.90291 | 0.90311 | 1 | 15464 | 0 | −0.45828 |
| EIF3A | 4 | 0.00024471 | 0.0008877 | 0.091909 | 172 | 3 | −0.85192 | 0.83888 | 0.83897 | 1 | 14226 | 0 | −0.85192 |
| MOCS3 | 4 | 0.000246 | 0.00089099 | 0.091909 | 173 | 4 | −1.2452 | 0.99875 | 0.99874 | 1 | 17870 | 0 | −1.2452 |
| PSMC2 | 4 | 0.00025809 | 0.00093596 | 0.095166 | 174 | 4 | −0.58058 | 0.99971 | 0.99971 | 1 | 17949 | 0 | −0.58058 |
| MGRN1 | 4 | 0.00026015 | 0.000942 | 0.095166 | 175 | 3 | −0.53717 | 0.98445 | 0.98453 | 1 | 17348 | 0 | −0.53717 |
| SRC | 4 | 0.00026018 | 0.000942 | 0.095166 | 176 | 4 | −0.45381 | 0.99974 | 0.99974 | 1 | 17955 | 0 | −0.45381 |
| USP21 | 4 | 0.00026077 | 0.00094364 | 0.095166 | 177 | 4 | −0.42865 | 0.99968 | 0.99968 | 1 | 17946 | 0 | −0.42865 |
| GGNBP2 | 3 | 0.00026371 | 0.00079227 | 0.084627 | 178 | 3 | −0.853 | 0.99974 | 0.99972 | 1 | 17954 | 0 | −0.853 |
| ITGAV | 4 | 0.00026373 | 0.00095516 | 0.095263 | 179 | 3 | −1.5887 | 0.95404 | 0.95417 | 1 | 16549 | 0 | −1.5887 |
| PSMB4 | 4 | 0.0002639 | 0.00095516 | 0.095263 | 180 | 4 | −1.0404 | 0.99974 | 0.99974 | 1 | 17953 | 0 | −1.0404 |
| TOMM7 | 4 | 0.00027171 | 0.00097875 | 0.097079 | 181 | 4 | −0.52553 | 0.99973 | 0.99973 | 1 | 17951 | 0 | −0.52553 |
| ASCC3 | 4 | 0.00027527 | 0.00099355 | 0.09753 | 182 | 4 | −0.47714 | 0.99939 | 0.9994 | 1 | 17920 | 0 | −0.47714 |
| NCOR1 | 4 | 0.00027543 | 0.0009941 | 0.09753 | 183 | 4 | −0.33492 | 0.99972 | 0.99973 | 1 | 17950 | 0 | −0.33492 |
| CNOT1 | 4 | 0.00028342 | 0.0010193 | 0.099465 | 184 | 3 | −0.95692 | 0.98833 | 0.9884 | 1 | 17473 | 0 | −0.95692 |
| DCAF7 | 1 | 0.00050116 | 0.00050103 | 0.063249 | 230 | 1 | −1.052 | 0.9995 | 0.99952 | 1 | 17928 | 0 | −1.052 |

H23 MRTX SL Gene FDR 0.1

| | | | | | | | | | | | | | |
|---|---|---|---|---|---|---|---|---|---|---|---|---|---|
| RNASEH2A | 4 | 9.54E−12 | 2.74E−07 | 0.000381 | 1 | 4 | −4.325 | 1 | 1 | 1 | 18053 | 0 | −4.325 |
| RTCB | 4 | 1.38E−11 | 2.74E−07 | 0.000381 | 2 | 4 | −2.3817 | 1 | 1 | 1 | 18052 | 0 | −2.3817 |
| C14orf80 | 4 | 5.06E−09 | 2.74E−07 | 0.000381 | 3 | 4 | −4.1074 | 1 | 1 | 1 | 18051 | 0 | −4.1074 |
| WRB | 4 | 1.17E−08 | 2.74E−07 | 0.000381 | 4 | 4 | −2.8881 | 1 | 1 | 1 | 18050 | 0 | −2.8881 |
| BUB3 | 4 | 1.25E−08 | 2.74E−07 | 0.000381 | 5 | 4 | −3.9325 | 1 | 1 | 1 | 18049 | 0 | −3.9325 |
| SOD2 | 4 | 1.36E−08 | 2.74E−07 | 0.000381 | 6 | 4 | −2.7857 | 0.99989 | 0.99989 | 1 | 17903 | 0 | −2.7857 |
| ALG1 | 4 | 1.95E−08 | 2.74E−07 | 0.000381 | 7 | 4 | −3.2865 | 1 | 1 | 1 | 18048 | 0 | −3.2865 |
| VHL | 4 | 3.24E−08 | 2.74E−07 | 0.000381 | 8 | 4 | −2.6264 | 1 | 1 | 1 | 18047 | 0 | −2.6264 |
| N6AMT1 | 4 | 5.27E−08 | 2.74E−07 | 0.000381 | 9 | 4 | −3.7445 | 1 | 1 | 1 | 18046 | 0 | −3.7445 |
| DPH1 | 4 | 9.28E−08 | 2.74E−07 | 0.000381 | 10 | 4 | −2.3602 | 1 | 1 | 1 | 18045 | 0 | −2.3602 |
| IDH3A | 4 | 1.21E−07 | 2.74E−07 | 0.000381 | 11 | 4 | −3.4713 | 1 | 1 | 1 | 18044 | 0 | −3.4713 |
| SMG9 | 4 | 1.48E−07 | 2.74E−07 | 0.000381 | 12 | 4 | −1.7289 | 1 | 1 | 1 | 18043 | 0 | −1.7289 |
| RNMT | 4 | 1.58E−07 | 2.74E−07 | 0.000381 | 13 | 4 | −2.982 | 1 | 1 | 1 | 18042 | 0 | −2.982 |
| DOHH | 4 | 1.85E−07 | 8.23E−07 | 0.00099 | 14 | 4 | −2.4973 | 1 | 1 | 1 | 18040 | 0 | −2.4973 |
| DOLK | 4 | 2.46E−07 | 8.23E−07 | 0.00099 | 15 | 4 | −5.3823 | 1 | 1 | 1 | 18041 | 0 | −5.3823 |
| DTYMK | 4 | 4.57E−07 | 1.37E−06 | 0.001444 | 16 | 4 | −3.2286 | 1 | 1 | 1 | 18039 | 0 | −3.2286 |
| GATA5 | 4 | 4.97E−07 | 1.37E−06 | 0.001444 | 17 | 4 | −2.0641 | 1 | 1 | 1 | 18038 | 0 | −2.0641 |
| SDHB | 4 | 5.20E−07 | 1.92E−06 | 0.001444 | 18 | 4 | −2.6747 | 1 | 1 | 1 | 18037 | 0 | −2.6747 |
| C11orf57 | 4 | 5.79E−07 | 1.92E−06 | 0.001444 | 19 | 4 | −1.8551 | 1 | 1 | 1 | 18036 | 0 | −1.8551 |
| MTG2 | 4 | 6.76E−07 | 1.92E−06 | 0.001444 | 20 | 4 | −2.2587 | 1 | 1 | 1 | 18035 | 0 | −2.2587 |
| MBTPS2 | 4 | 6.93E−07 | 1.92E−06 | 0.001444 | 21 | 3 | −2.0657 | 0.21288 | 0.39945 | 0.984062 | 7252 | 1 | −2.0657 |
| DDOST | 4 | 6.96E−07 | 1.92E−06 | 0.001444 | 22 | 4 | −1.7775 | 1 | 1 | 1 | 18034 | 0 | −1.7775 |
| JMJD6 | 4 | 7.03E−07 | 1.92E−06 | 0.001444 | 23 | 4 | −2.6172 | 1 | 1 | 1 | 18033 | 0 | −2.6172 |
| ARMC5 | 4 | 7.25E−07 | 1.92E−06 | 0.001444 | 24 | 4 | −2.9467 | 0.9891 | 0.98922 | 1 | 16992 | 0 | −2.9467 |
| PAXIP1 | 4 | 7.83E−07 | 2.47E−06 | 0.00165 | 25 | 4 | −2.7465 | 1 | 1 | 1 | 18032 | 0 | −2.7465 |
| OTUD6B | 4 | 8.16E−07 | 2.47E−06 | 0.00165 | 26 | 4 | −1.8293 | 1 | 1 | 1 | 18031 | 0 | −1.8293 |
| WDR77 | 4 | 8.83E−07 | 2.47E−06 | 0.00165 | 27 | 4 | −4.5655 | 1 | 1 | 1 | 18030 | 0 | −4.5655 |
| DBR1 | 4 | 9.68E−07 | 3.02E−06 | 0.001945 | 28 | 4 | −3.0064 | 1 | 1 | 1 | 18029 | 0 | −3.0064 |
| POP5 | 4 | 1.05E−06 | 3.56E−06 | 0.002145 | 29 | 4 | −2.2451 | 0.99995 | 0.99995 | 1 | 17954 | 0 | −2.2451 |
| NAA20 | 4 | 1.09E−06 | 3.56E−06 | 0.002145 | 30 | 3 | −3.5954 | 0.92085 | 0.92063 | 1 | 15226 | 0 | −3.5954 |
| ALG2 | 4 | 1.44E−06 | 4.66E−06 | 0.002338 | 31 | 4 | −4.8043 | 1 | 1 | 1 | 18028 | 0 | −4.8043 |
| RTEL1 | 4 | 1.49E−06 | 4.66E−06 | 0.002338 | 32 | 4 | −4.4717 | 1 | 1 | 1 | 18027 | 0 | −4.4717 |
| XRCC2 | 4 | 1.59E−06 | 4.66E−06 | 0.002338 | 33 | 4 | −2.3175 | 0.97654 | 0.97659 | 1 | 16533 | 0 | −2.3175 |
| INTS10 | 4 | 1.66E−06 | 4.66E−06 | 0.002338 | 34 | 4 | −1.3758 | 1 | 1 | 1 | 18026 | 0 | −1.3758 |
| CINP | 4 | 1.68E−06 | 4.66E−06 | 0.002338 | 35 | 4 | −3.2177 | 0.99789 | 0.99792 | 1 | 17557 | 0 | −3.2177 |
| MTG1 | 4 | 1.69E−06 | 4.66E−06 | 0.002338 | 36 | 4 | −2.2531 | 0.99999 | 0.99999 | 1 | 18004 | 0 | −2.2531 |
| L3MBTL2 | 4 | 1.73E−06 | 5.21E−06 | 0.002351 | 37 | 4 | −1.6238 | 1 | 1 | 1 | 18025 | 0 | −1.6238 |
| PPP2R4 | 4 | 1.81E−06 | 5.21E−06 | 0.002351 | 38 | 4 | −3.3086 | 1 | 1 | 1 | 18024 | 0 | −3.3086 |
| NOC4L | 4 | 1.91E−06 | 5.21E−06 | 0.002351 | 39 | 4 | −3.9038 | 1 | 1 | 1 | 18023 | 0 | −3.9038 |
| RCL1 | 4 | 2.07E−06 | 5.21E−06 | 0.002351 | 40 | 4 | −2.6376 | 1 | 1 | 1 | 18022 | 0 | −2.6376 |
| RIOK2 | 4 | 2.28E−06 | 5.76E−06 | 0.002475 | 41 | 4 | −2.7007 | 1 | 1 | 1 | 18021 | 0 | −2.7007 |
| MRPL28 | 4 | 2.30E−06 | 5.76E−06 | 0.002475 | 42 | 4 | −3.3185 | 1 | 1 | 1 | 18020 | 0 | −3.3185 |
| ARFRP1 | 4 | 2.48E−06 | 6.31E−06 | 0.002588 | 43 | 4 | −2.3446 | 0.99988 | 0.99988 | 1 | 17894 | 0 | −2.3446 |
| GEMIN7 | 4 | 2.51E−06 | 6.31E−06 | 0.002588 | 44 | 4 | −2.2853 | 0.99996 | 0.99996 | 1 | 17971 | 0 | −2.2853 |
| PSMG4 | 4 | 2.58E−06 | 6.86E−06 | 0.00275 | 45 | 3 | −2.15 | 0.73286 | 0.79957 | 1 | 13044 | 1 | −2.15 |
| GTPBP10 | 4 | 2.76E−06 | 8.78E−06 | 0.003444 | 46 | 4 | −2.1725 | 0.99999 | 0.99999 | 1 | 17997 | 0 | −2.1725 |
| EXT1 | 4 | 3.12E−06 | 1.07E−05 | 0.004108 | 47 | 4 | −2.2427 | 1 | 1 | 1 | 18019 | 0 | −2.2427 |
| TGIF1 | 4 | 3.39E−06 | 1.12E−05 | 0.004229 | 48 | 4 | −2.8487 | 1 | 1 | 1 | 18018 | 0 | −2.8487 |
| TYMS | 4 | 3.66E−06 | 1.18E−05 | 0.00423 | 49 | 4 | −2.2649 | 0.98783 | 0.98792 | 1 | 16938 | 0 | −2.2649 |
| SRP14 | 4 | 3.84E−06 | 1.23E−05 | 0.00423 | 50 | 4 | −3.49 | 1 | 1 | 1 | 18017 | 0 | −3.49 |
| VRK1 | 4 | 3.90E−06 | 1.23E−05 | 0.00423 | 51 | 3 | −1.9561 | 0.34717 | 0.5299 | 1 | 9099 | 1 | −1.9561 |
| GNB2L1 | 4 | 4.08E−06 | 1.23E−05 | 0.00423 | 52 | 4 | −2.9147 | 1 | 1 | 1 | 18016 | 0 | −2.9147 |
| RIC8A | 4 | 4.16E−06 | 1.29E−05 | 0.00423 | 53 | 4 | −1.5366 | 1 | 1 | 1 | 18015 | 0 | −1.5366 |
| TFB2M | 4 | 4.17E−06 | 1.29E−05 | 0.00423 | 54 | 4 | −1.3531 | 1 | 1 | 1 | 18014 | 0 | −1.3531 |
| FDXR | 4 | 4.18E−06 | 1.29E−05 | 0.00423 | 55 | 4 | −2.4996 | 0.99998 | 0.99998 | 1 | 17986 | 0 | −2.4996 |
| ARL2 | 4 | 4.39E−06 | 1.40E−05 | 0.004508 | 56 | 4 | −2.7189 | 1 | 1 | 1 | 18013 | 0 | −2.7189 |
| ORC5 | 4 | 4.45E−06 | 1.45E−05 | 0.004603 | 57 | 4 | −1.7446 | 1 | 1 | 1 | 18012 | 0 | −1.7446 |
| C7orf55-LUC7L2 | 4 | 5.03E−06 | 1.67E−05 | 0.005198 | 58 | 4 | −2.6931 | 0.99999 | 1 | 1 | 18011 | 0 | −2.6931 |
| MCMBP | 4 | 5.12E−06 | 1.73E−05 | 0.005198 | 59 | 4 | −4.3854 | 0.99903 | 0.99902 | 1 | 17702 | 0 | −4.3854 |
| HSD17B10 | 4 | 5.13E−06 | 1.73E−05 | 0.005198 | 60 | 4 | −3.4918 | 0.99999 | 1 | 1 | 18010 | 0 | −3.4918 |
| SEC63 | 4 | 5.57E−06 | 1.84E−05 | 0.005437 | 61 | 4 | −2.2069 | 0.99997 | 0.99997 | 1 | 17977 | 0 | −2.2069 |
| PTK2 | 4 | 5.87E−06 | 1.95E−05 | 0.005669 | 62 | 4 | −2.6007 | 0.99999 | 1 | 1 | 18009 | 0 | −2.6007 |
| E4F1 | 4 | 6.81E−06 | 2.11E−05 | 0.006051 | 63 | 4 | −1.8551 | 0.99992 | 0.99992 | 1 | 17931 | 0 | −1.8551 |
| C9orf114 | 4 | 7.56E−06 | 2.33E−05 | 0.006575 | 64 | 4 | −2.2167 | 0.99999 | 0.99999 | 1 | 18008 | 0 | −2.2167 |
| WDR73 | 4 | 7.82E−06 | 2.50E−05 | 0.006931 | 65 | 3 | −3.7966 | 0.88073 | 0.88388 | 1 | 14504 | 1 | −3.7966 |
| PDSS1 | 4 | 8.10E−06 | 2.66E−05 | 0.007276 | 66 | 4 | −1.6052 | 0.99999 | 0.99999 | 1 | 18007 | 0 | −1.6052 |
| FARS2 | 4 | 8.41E−06 | 2.77E−05 | 0.007353 | 67 | 4 | −2.5173 | 0.99876 | 0.99875 | 1 | 17666 | 0 | −2.5173 |
| TCEB2 | 4 | 8.47E−06 | 2.77E−05 | 0.007353 | 68 | 4 | −4.6878 | 0.99999 | 0.99999 | 1 | 18006 | 0 | −4.6878 |
| OGT | 4 | 8.72E−06 | 2.91E−05 | 0.007605 | 69 | 4 | −1.6816 | 0.9985 | 0.99852 | 1 | 17641 | 0 | −1.6816 |
| DDX59 | 3 | 8.76E−06 | 3.37E−05 | 0.008084 | 70 | 3 | −2.7859 | 0.99999 | 0.99999 | 1 | 18005 | 0 | −2.7859 |
| EMC1 | 4 | 8.96E−06 | 3.04E−05 | 0.00785 | 71 | 4 | −3.6346 | 0.99999 | 0.99999 | 1 | 18003 | 0 | −3.6346 |
| C7orf25 | 4 | 9.28E−06 | 3.21E−05 | 0.008084 | 72 | 4 | −4.3504 | 0.99999 | 0.99999 | 1 | 18002 | 0 | −4.3504 |
| DENND2D | 4 | 9.37E−06 | 3.32E−05 | 0.008084 | 73 | 4 | −2.1379 | 0.99999 | 0.99999 | 1 | 18001 | 0 | −2.1379 |
| BCCIP | 4 | 9.50E−06 | 3.43E−05 | 0.008084 | 74 | 4 | −2.2388 | 0.99748 | 0.99751 | 1 | 17517 | 0 | −2.2388 |
| GGPS1 | 4 | 9.93E−06 | 3.54E−05 | 0.008084 | 75 | 4 | −1.8006 | 0.99889 | 0.99888 | 1 | 17685 | 0 | −1.8006 |
| METTL3 | 4 | 1.01E−05 | 3.54E−05 | 0.008084 | 76 | 4 | −1.7993 | 0.98183 | 0.98192 | 1 | 16693 | 0 | −1.7993 |
| TBCB | 4 | 1.02E−05 | 3.54E−05 | 0.008084 | 77 | 4 | −3.9054 | 0.99999 | 0.99999 | 1 | 18000 | 0 | −3.9054 |
| CCS | 4 | 1.02E−05 | 3.54E−05 | 0.008084 | 78 | 4 | −2.1558 | 0.99999 | 0.99999 | 1 | 17999 | 0 | −2.1558 |
| KREMEN2 | 4 | 1.03E−05 | 3.54E−05 | 0.008084 | 79 | 4 | −2.2038 | 0.99999 | 0.99999 | 1 | 17998 | 0 | −2.2038 |
| CDIPT | 4 | 1.11E−05 | 3.70E−05 | 0.008354 | 80 | 4 | −5.2679 | 0.99999 | 0.99999 | 1 | 17996 | 0 | −5.2679 |
| RAPGEF1 | 4 | 1.15E−05 | 3.92E−05 | 0.00874 | 81 | 3 | −1.7406 | 0.75172 | 0.80807 | 1 | 13186 | 1 | −1.7406 |
| MTOR | 4 | 1.18E−05 | 4.20E−05 | 0.009237 | 82 | 4 | −2.9795 | 0.99999 | 0.99999 | 1 | 17995 | 0 | −2.9795 |
| ARPC4 | 4 | 1.20E−05 | 4.31E−05 | 0.009364 | 83 | 4 | −1.5867 | 0.99999 | 0.99999 | 1 | 17994 | 0 | −1.5867 |
| PFN1 | 4 | 1.27E−05 | 4.69E−05 | 0.010078 | 84 | 4 | −2.0809 | 0.99999 | 0.99999 | 1 | 17993 | 0 | −2.0809 |
| PARS2 | 4 | 1.37E−05 | 4.96E−05 | 0.010413 | 85 | 4 | −1.8533 | 0.99999 | 0.99999 | 1 | 17992 | 0 | −1.8533 |
| NHLRC2 | 4 | 1.37E−05 | 5.02E−05 | 0.010413 | 86 | 4 | −1.927 | 0.99999 | 0.99999 | 1 | 17991 | 0 | −1.927 |
| PKN2 | 4 | 1.40E−05 | 5.02E−05 | 0.010413 | 87 | 4 | −1.9813 | 0.99418 | 0.9943 | 1 | 17265 | 0 | −1.9813 |
| TEN1 | 4 | 1.48E−05 | 5.29E−05 | 0.010857 | 88 | 4 | −2.7549 | 0.99997 | 0.99997 | 1 | 17975 | 0 | −2.7549 |
| DHX33 | 4 | 1.54E−05 | 5.46E−05 | 0.011069 | 89 | 3 | −3.1932 | 0.81097 | 0.8379 | 1 | 13658 | 1 | −3.1932 |
| OPA1 | 4 | 1.69E−05 | 6.33E−05 | 0.012706 | 90 | 4 | −1.4416 | 0.99998 | 0.99998 | 1 | 17990 | 0 | −1.4416 |
| COG1 | 4 | 1.77E−05 | 6.55E−05 | 0.013002 | 91 | 4 | −2.2495 | 0.99998 | 0.99998 | 1 | 17989 | 0 | −2.2495 |
| MOGS | 4 | 1.84E−05 | 6.83E−05 | 0.013399 | 92 | 4 | −1.6887 | 0.99998 | 0.99998 | 1 | 17988 | 0 | −1.6887 |
| C18orf21 | 4 | 1.90E−05 | 7.10E−05 | 0.013787 | 93 | 4 | −2.8181 | 0.99998 | 0.99998 | 1 | 17987 | 0 | −2.8181 |
| MRPS18A | 4 | 1.98E−05 | 7.32E−05 | 0.014018 | 94 | 4 | −2.4166 | 0.99998 | 0.99998 | 1 | 17985 | 0 | −2.4166 |
| PGD | 4 | 1.98E−05 | 7.38E−05 | 0.014018 | 95 | 4 | −2.0025 | 0.99993 | 0.99993 | 1 | 17935 | 0 | −2.0025 |
| ORC3 | 4 | 2.06E−05 | 7.60E−05 | 0.014284 | 96 | 3 | −1.4197 | 0.85932 | 0.86809 | 1 | 14194 | 1 | −1.4197 |
| OGFR | 4 | 2.11E−05 | 7.76E−05 | 0.014443 | 97 | 4 | −1.8506 | 0.99998 | 0.99998 | 1 | 17984 | 0 | −1.8506 |
| RABGGTB | 4 | 2.16E−05 | 7.93E−05 | 0.014599 | 98 | 4 | −3.374 | 0.99997 | 0.99997 | 1 | 17980 | 0 | −3.374 |
| IKBKAP | 4 | 2.42E−05 | 9.02E−05 | 0.016266 | 99 | 4 | −2.4402 | 0.99511 | 0.9952 | 1 | 17329 | 0 | −2.4402 |
| TBCE | 4 | 2.48E−05 | 9.30E−05 | 0.016266 | 100 | 4 | −1.8381 | 0.99998 | 0.99997 | 1 | 17983 | 0 | −1.8381 |
| ELP5 | 4 | 2.49E−05 | 9.30E−05 | 0.016266 | 101 | 4 | −3.2549 | 0.99998 | 0.99997 | 1 | 17982 | 0 | −3.2549 |
| KIAA1324 | 4 | 2.52E−05 | 9.35E−05 | 0.016266 | 102 | 3 | −1.5026 | 0.95451 | 0.95436 | 1 | 15942 | 0 | −1.5026 |
| ATP6V1F | 4 | 2.56E−05 | 9.41E−05 | 0.016266 | 103 | 4 | −2.1159 | 0.99894 | 0.99893 | 1 | 17691 | 0 | −2.1159 |
| PHF6 | 4 | 2.56E−05 | 9.41E−05 | 0.016266 | 104 | 4 | −2.4956 | 0.99989 | 0.9999 | 1 | 17904 | 0 | −2.4956 |
| TRIT1 | 4 | 2.59E−05 | 9.46E−05 | 0.016266 | 105 | 4 | −2.6356 | 0.99997 | 0.99997 | 1 | 17981 | 0 | −2.6356 |
| PIK3C3 | 4 | 2.69E−05 | 9.79E−05 | 0.016517 | 106 | 4 | −2.5172 | 0.99997 | 0.99997 | 1 | 17979 | 0 | −2.5172 |
| PEAR1 | 4 | 2.70E−05 | 9.79E−05 | 0.016517 | 107 | 4 | −1.6364 | 0.99997 | 0.99997 | 1 | 17978 | 0 | −1.6364 |
| ZC3HC1 | 4 | 2.87E−05 | 0.00010612 | 0.017577 | 108 | 4 | −2.0677 | 0.9883 | 0.98839 | 1 | 16956 | 0 | −2.0677 |
| DAP3 | 4 | 2.87E−05 | 0.00010612 | 0.017577 | 109 | 4 | −3.013 | 0.99997 | 0.99997 | 1 | 17976 | 0 | −3.013 |
| COX5B | 4 | 3.24E−05 | 0.00011654 | 0.019127 | 110 | 4 | −2.7515 | 0.99997 | 0.99997 | 1 | 17974 | 0 | −2.7515 |
| PPP6C | 4 | 3.39E−05 | 0.00012203 | 0.019758 | 111 | 4 | −2.548 | 0.99997 | 0.99997 | 1 | 17973 | 0 | −2.548 |
| PSTK | 4 | 3.40E−05 | 0.00012258 | 0.019758 | 112 | 4 | −3.4884 | 0.95661 | 0.95649 | 1 | 15987 | 0 | −3.4884 |
| NUP188 | 4 | 3.51E−05 | 0.00012861 | 0.020547 | 113 | 4 | −2.6872 | 0.99991 | 0.99992 | 1 | 17925 | 0 | −2.6872 |
| KDM2A | 4 | 3.57E−05 | 0.00013135 | 0.020706 | 114 | 4 | −2.4557 | 0.99996 | 0.99996 | 1 | 17972 | 0 | −2.4557 |
| SAE1 | 4 | 3.61E−05 | 0.0001319 | 0.020706 | 115 | 4 | −3.112 | 0.99986 | 0.99987 | 1 | 17891 | 0 | −3.112 |
| ELAC2 | 4 | 3.72E−05 | 0.00013738 | 0.021103 | 116 | 4 | −1.6371 | 0.97499 | 0.97498 | 1 | 16485 | 0 | −1.6371 |
| VMA21 | 4 | 3.73E−05 | 0.00013793 | 0.021103 | 117 | 4 | −1.1034 | 0.99996 | 0.99996 | 1 | 17970 | 0 | −1.1034 |
| OR6P1 | 4 | 3.75E−05 | 0.00013793 | 0.021103 | 118 | 4 | −1.0259 | 0.99996 | 0.99996 | 1 | 17969 | 0 | −1.0259 |
| NGDN | 4 | 3.82E−05 | 0.00014287 | 0.021109 | 119 | 4 | −3.7376 | 0.99996 | 0.99996 | 1 | 17968 | 0 | −3.7376 |
| RPP21 | 4 | 3.85E−05 | 0.00014342 | 0.021109 | 120 | 4 | −2.9143 | 0.99996 | 0.99996 | 1 | 17967 | 0 | −2.9143 |
| NOLC1 | 4 | 3.89E−05 | 0.00014451 | 0.021109 | 121 | 4 | −1.8516 | 0.99996 | 0.99996 | 1 | 17966 | 0 | −1.8516 |
| ERCC2 | 4 | 3.90E−05 | 0.00014561 | 0.021109 | 122 | 4 | −2.3551 | 0.99996 | 0.99996 | 1 | 17965 | 0 | −2.3551 |
| NDNL2 | 4 | 3.91E−05 | 0.00014561 | 0.021109 | 123 | 4 | −2.3803 | 0.99996 | 0.99996 | 1 | 17964 | 0 | −2.3803 |
| SLC7A5 | 4 | 3.93E−05 | 0.00014561 | 0.021109 | 124 | 4 | −1.8676 | 0.99996 | 0.99996 | 1 | 17963 | 0 | −1.8676 |
| ASUN | 4 | 3.94E−05 | 0.00014616 | 0.021109 | 125 | 4 | −1.9627 | 0.99996 | 0.99996 | 1 | 17962 | 0 | −1.9627 |
| EXOC2 | 4 | 4.04E−05 | 0.00015055 | 0.02157 | 126 | 4 | −1.6999 | 0.9999 | 0.99991 | 1 | 17919 | 0 | −1.6999 |
| TRMT61A | 4 | 4.24E−05 | 0.00015822 | 0.022168 | 127 | 3 | −2.6335 | 0.64771 | 0.76638 | 1 | 12535 | 1 | −2.6335 |
| ELP3 | 4 | 4.37E−05 | 0.00016152 | 0.022168 | 128 | 4 | −1.7399 | 0.98279 | 0.98287 | 1 | 16733 | 0 | −1.7399 |
| ANKRD49 | 4 | 4.41E−05 | 0.00016426 | 0.022168 | 129 | 4 | −1.7616 | 0.99993 | 0.99994 | 1 | 17939 | 0 | −1.7616 |
| KDM8 | 4 | 4.41E−05 | 0.00016426 | 0.022168 | 130 | 3 | −1.3411 | 0.59043 | 0.73164 | 1 | 12018 | 1 | −1.3411 |
| AIFM1 | 4 | 4.42E−05 | 0.00016426 | 0.022168 | 131 | 4 | −1.9096 | 0.99888 | 0.99886 | 1 | 17683 | 0 | −1.9096 |
| PHF23 | 4 | 4.50E−05 | 0.00016535 | 0.022168 | 132 | 4 | −1.346 | 0.99995 | 0.99996 | 1 | 17961 | 0 | −1.346 |
| UTP23 | 4 | 4.58E−05 | 0.00016645 | 0.022168 | 133 | 3 | −2.4217 | 0.76923 | 0.81628 | 1 | 13317 | 1 | −2.4217 |
| SLC6A17 | 4 | 4.58E−05 | 0.00016645 | 0.022168 | 134 | 4 | −1.4876 | 0.99995 | 0.99996 | 1 | 17960 | 0 | −1.4876 |
| ELP4 | 4 | 4.60E−05 | 0.000167 | 0.022168 | 135 | 4 | −2.1033 | 0.99976 | 0.99977 | 1 | 17850 | 0 | −2.1033 |
| CCNC | 4 | 4.60E−05 | 0.000167 | 0.022168 | 136 | 4 | −1.5464 | 0.99995 | 0.99996 | 1 | 17959 | 0 | −1.5464 |
| GUK1 | 4 | 4.67E−05 | 0.00017139 | 0.022421 | 137 | 4 | −2.4728 | 0.99995 | 0.99996 | 1 | 17958 | 0 | −2.4728 |
| TAF13 | 4 | 4.67E−05 | 0.00017139 | 0.022421 | 138 | 4 | −2.503 | 0.99962 | 0.99964 | 1 | 17814 | 0 | −2.503 |
| RBM15 | 4 | 4.76E−05 | 0.00017523 | 0.022758 | 139 | 4 | −1.5718 | 0.99995 | 0.99996 | 1 | 17957 | 0 | −1.5718 |
| GFM1 | 4 | 4.97E−05 | 0.00018455 | 0.023798 | 140 | 4 | −1.6873 | 0.99977 | 0.99978 | 1 | 17858 | 0 | −1.6873 |
| OTUD5 | 4 | 5.00E−05 | 0.0001862 | 0.02384 | 141 | 4 | −1.9032 | 0.99995 | 0.99995 | 1 | 17956 | 0 | −1.9032 |
| SMG7 | 4 | 5.03E−05 | 0.00018784 | 0.023881 | 142 | 3 | −2.0565 | 0.86483 | 0.87201 | 1 | 14275 | 1 | −2.0565 |
| TSEN54 | 4 | 5.20E−05 | 0.00019278 | 0.024005 | 143 | 4 | −2.4994 | 0.99955 | 0.99957 | 1 | 17798 | 0 | −2.4994 |
| AGXT2 | 4 | 5.23E−05 | 0.00019333 | 0.024005 | 144 | 4 | −0.90864 | 0.99995 | 0.99995 | 1 | 17955 | 0 | −0.90864 |
| AP2M1 | 4 | 5.33E−05 | 0.00019607 | 0.024005 | 145 | 4 | −1.4739 | 0.97917 | 0.97926 | 1 | 16602 | 0 | −1.4739 |
| FNTB | 4 | 5.37E−05 | 0.00019716 | 0.024005 | 146 | 4 | −3.5578 | 0.99989 | 0.9999 | 1 | 17905 | 0 | −3.5578 |
| MRPL47 | 4 | 5.42E−05 | 0.00019826 | 0.024005 | 147 | 4 | −1.4447 | 0.99995 | 0.99995 | 1 | 17953 | 0 | −1.4447 |
| CDK7 | 4 | 5.44E−05 | 0.00019881 | 0.024005 | 148 | 4 | −2.2305 | 0.99995 | 0.99995 | 1 | 17952 | 0 | −2.2305 |
| TSEN2 | 4 | 5.45E−05 | 0.00019936 | 0.024005 | 149 | 4 | −1.8068 | 0.9999 | 0.99991 | 1 | 17916 | 0 | −1.8068 |
| GAPDH | 4 | 5.47E−05 | 0.00020045 | 0.024005 | 150 | 4 | −2.417 | 0.99971 | 0.99972 | 1 | 17835 | 0 | −2.417 |
| CRK | 4 | 5.51E−05 | 0.00020265 | 0.024005 | 151 | 4 | −1.6978 | 0.99994 | 0.99995 | 1 | 17951 | 0 | −1.6978 |
| PFDN6 | 4 | 5.62E−05 | 0.00020539 | 0.024005 | 152 | 4 | −1.6322 | 0.99994 | 0.99995 | 1 | 17950 | 0 | −1.6322 |
| POLR3K | 4 | 5.63E−05 | 0.00020649 | 0.024005 | 153 | 4 | −1.8789 | 0.98476 | 0.98482 | 1 | 16803 | 0 | −1.8789 |
| ZNF259 | 4 | 5.65E−05 | 0.00020896 | 0.024005 | 154 | 3 | −2.447 | 0.87799 | 0.88182 | 1 | 14458 | 1 | −2.447 |
| AASDHPPT | 4 | 5.68E−05 | 0.00020978 | 0.024005 | 155 | 3 | −1.1912 | 0.91472 | 0.91452 | 1 | 15116 | 0 | −1.1912 |
| WBSCR16 | 4 | 5.71E−05 | 0.00021088 | 0.024005 | 156 | 4 | −1.6428 | 0.99994 | 0.99995 | 1 | 17949 | 0 | −1.6428 |
| ELP2 | 4 | 5.74E−05 | 0.00021115 | 0.024005 | 157 | 4 | −1.541 | 0.99918 | 0.99917 | 1 | 17724 | 0 | −1.541 |
| LIG4 | 4 | 5.75E−05 | 0.00021142 | 0.024005 | 158 | 4 | −1.568 | 0.99986 | 0.99987 | 1 | 17888 | 0 | −1.568 |
| GSG2 | 4 | 5.75E−05 | 0.00021142 | 0.024005 | 159 | 4 | −2.4547 | 0.99994 | 0.99995 | 1 | 17948 | 0 | −2.4547 |
| ILF3 | 4 | 5.88E−05 | 0.00021718 | 0.024505 | 160 | 4 | −1.5561 | 0.99994 | 0.99995 | 1 | 17947 | 0 | −1.5561 |
| CHTF8 | 4 | 5.98E−05 | 0.00021965 | 0.024539 | 161 | 4 | −2.4543 | 0.99964 | 0.99966 | 1 | 17821 | 0 | −2.4543 |
| ADRM1 | 4 | 6.02E−05 | 0.0002202 | 0.024539 | 162 | 4 | −2.1489 | 0.99994 | 0.99995 | 1 | 17946 | 0 | −2.1489 |
| BAK1 | 4 | 6.05E−05 | 0.00022239 | 0.024541 | 163 | 4 | −2.618 | 0.99944 | 0.99946 | 1 | 17774 | 0 | −2.618 |
| AHCYL2 | 4 | 6.06E−05 | 0.00022294 | 0.024541 | 164 | 3 | −1.3757 | 0.62289 | 0.75793 | 1 | 12393 | 1 | −1.3757 |
| BCS1L | 4 | 6.14E−05 | 0.00022623 | 0.024752 | 165 | 3 | −1.7123 | 0.94204 | 0.94191 | 1 | 15665 | 0 | −1.7123 |
| GMPS | 4 | 6.28E−05 | 0.00023446 | 0.025498 | 166 | 4 | −1.6931 | 0.99994 | 0.99995 | 1 | 17945 | 0 | −1.6931 |
| PPP4C | 4 | 6.46E−05 | 0.00024268 | 0.026235 | 167 | 4 | −3.3615 | 0.99994 | 0.99994 | 1 | 17944 | 0 | −3.3615 |
| RPN1 | 4 | 6.61E−05 | 0.00024707 | 0.026489 | 168 | 4 | −1.4044 | 0.99993 | 0.99994 | 1 | 17943 | 0 | −1.4044 |
| PDCD6IP | 4 | 6.67E−05 | 0.00024927 | 0.026489 | 169 | 4 | −1.5793 | 0.99993 | 0.99994 | 1 | 17942 | 0 | −1.5793 |
| CDC123 | 4 | 6.70E−05 | 0.00025091 | 0.026489 | 170 | 4 | −2.1358 | 0.99993 | 0.99994 | 1 | 17941 | 0 | −2.1358 |
| EP300 | 4 | 6.70E−05 | 0.00025091 | 0.026489 | 171 | 4 | −1.5411 | 0.97907 | 0.97917 | 1 | 16598 | 0 | −1.5411 |
| HYOU1 | 4 | 6.78E−05 | 0.00025311 | 0.026566 | 172 | 4 | −1.7692 | 0.99685 | 0.99689 | 1 | 17454 | 0 | −1.7692 |
| TPI1 | 4 | 6.88E−05 | 0.00025749 | 0.026716 | 173 | 4 | −3.0244 | 0.99993 | 0.99994 | 1 | 17940 | 0 | −3.0244 |
| URB1 | 4 | 6.88E−05 | 0.00025749 | 0.026716 | 174 | 3 | −1.8097 | 0.95363 | 0.95349 | 1 | 15927 | 0 | −1.8097 |
| ALG13 | 4 | 7.02E−05 | 0.00026078 | 0.026902 | 175 | 3 | −3.0903 | 0.66715 | 0.77331 | 1 | 12635 | 1 | −3.0903 |
| SRP9 | 4 | 7.05E−05 | 0.00026298 | 0.026965 | 176 | 4 | −2.1637 | 0.99993 | 0.99994 | 1 | 17938 | 0 | −2.1637 |
| THOC7 | 4 | 7.17E−05 | 0.00026572 | 0.026965 | 177 | 4 | −2.086 | 0.99993 | 0.99994 | 1 | 17937 | 0 | −2.086 |
| PRMT1 | 4 | 7.23E−05 | 0.00026682 | 0.026965 | 178 | 4 | −3.3129 | 0.99436 | 0.99448 | 1 | 17275 | 0 | −3.3129 |
| STT3A | 4 | 7.29E−05 | 0.00026736 | 0.026965 | 179 | 4 | −1.7133 | 0.99993 | 0.99994 | 1 | 17936 | 0 | −1.7133 |
| VPS52 | 4 | 7.42E−05 | 0.00027285 | 0.027365 | 180 | 4 | −1.8887 | 0.99993 | 0.99993 | 1 | 17934 | 0 | −1.8887 |
| TAF2 | 4 | 7.53E−05 | 0.00028053 | 0.02798 | 181 | 4 | −1.8515 | 0.99952 | 0.99954 | 1 | 17790 | 0 | −1.8515 |
| PHIP | 4 | 7.72E−05 | 0.00028711 | 0.028479 | 182 | 4 | −1.6565 | 0.99992 | 0.99993 | 1 | 17933 | 0 | −1.6565 |
| HNRNPU | 4 | 7.76E−05 | 0.00028875 | 0.028486 | 183 | 4 | −2.751 | 0.99992 | 0.99993 | 1 | 17932 | 0 | −2.751 |
| ACTR6 | 4 | 8.06E−05 | 0.00030246 | 0.029626 | 184 | 4 | −2.5754 | 0.99991 | 0.99991 | 1 | 17921 | 0 | −2.5754 |
| UBE2H | 4 | 8.25E−05 | 0.00030795 | 0.029626 | 185 | 4 | −1.8091 | 0.99958 | 0.9996 | 1 | 17805 | 0 | −1.8091 |
| FAM96B | 4 | 8.35E−05 | 0.00031124 | 0.029626 | 186 | 4 | −4.5662 | 0.99992 | 0.99992 | 1 | 17930 | 0 | −4.5662 |
| ENO1 | 4 | 8.35E−05 | 0.00031124 | 0.029626 | 187 | 4 | −2.6786 | 0.999 | 0.99898 | 1 | 17699 | 0 | −2.6786 |
| XYLT2 | 4 | 8.40E−05 | 0.00031234 | 0.029626 | 188 | 4 | −2.4818 | 0.99992 | 0.99992 | 1 | 17929 | 0 | −2.4818 |
| IMP3 | 4 | 8.45E−05 | 0.00031398 | 0.029626 | 189 | 4 | −2.9595 | 0.99992 | 0.99992 | 1 | 17928 | 0 | −2.9595 |
| TCFL5 | 4 | 8.46E−05 | 0.00031398 | 0.029626 | 190 | 4 | −1.6942 | 0.99992 | 0.99992 | 1 | 17927 | 0 | −1.6942 |
| GZF1 | 4 | 8.53E−05 | 0.00031508 | 0.029626 | 191 | 4 | −1.3063 | 0.99991 | 0.99992 | 1 | 17926 | 0 | −1.3063 |
| NAE1 | 4 | 8.54E−05 | 0.00031508 | 0.029626 | 192 | 3 | −2.2648 | 0.94946 | 0.94937 | 1 | 15831 | 0 | −2.2648 |
| AP2S1 | 4 | 8.75E−05 | 0.00032056 | 0.029985 | 193 | 4 | −2.8836 | 0.9997 | 0.99971 | 1 | 17834 | 0 | −2.8836 |
| TBP | 4 | 8.85E−05 | 0.0003244 | 0.030188 | 194 | 4 | −3.5208 | 0.99991 | 0.99992 | 1 | 17924 | 0 | −3.5208 |
| TAF1C | 4 | 9.01E−05 | 0.00033098 | 0.030486 | 195 | 4 | −2.5013 | 0.99991 | 0.99992 | 1 | 17923 | 0 | −2.5013 |
| DPAGT1 | 4 | 9.03E−05 | 0.00033318 | 0.030532 | 196 | 4 | −2.7097 | 0.99922 | 0.99921 | 1 | 17734 | 0 | −2.7097 |
| TRMT6 | 4 | 9.08E−05 | 0.00033537 | 0.030578 | 197 | 4 | −2.9694 | 0.99977 | 0.99977 | 1 | 17853 | 0 | −2.9694 |
| NHP2 | 4 | 9.39E−05 | 0.00034579 | 0.031262 | 198 | 4 | −3.0583 | 0.99991 | 0.99991 | 1 | 17922 | 0 | −3.0583 |
| ACTR3 | 4 | 9.56E−05 | 0.00035402 | 0.031796 | 199 | 4 | −1.3082 | 0.9999 | 0.99991 | 1 | 17920 | 0 | −1.3082 |
| ALG11 | 4 | 9.63E−05 | 0.00035621 | 0.031835 | 200 | 4 | −1.4496 | 0.99881 | 0.99879 | 1 | 17673 | 0 | −1.4496 |
| TIMM22 | 4 | 9.74E−05 | 0.0003617 | 0.031953 | 201 | 4 | −1.7101 | 0.99724 | 0.99727 | 1 | 17488 | 0 | −1.7101 |
| XRCC3 | 4 | 9.76E−05 | 0.00036389 | 0.031953 | 202 | 4 | −1.7519 | 0.9999 | 0.99991 | 1 | 17918 | 0 | −1.7519 |
| TEX10 | 4 | 9.78E−05 | 0.00036499 | 0.031953 | 203 | 4 | −1.8751 | 0.99953 | 0.99955 | 1 | 17792 | 0 | −1.8751 |
| TOE1 | 4 | 9.81E−05 | 0.00036554 | 0.031953 | 204 | 4 | −1.8017 | 0.9999 | 0.99991 | 1 | 17917 | 0 | −1.8017 |
| NELFB | 4 | 9.89E−05 | 0.00036718 | 0.031953 | 205 | 4 | −3.2767 | 0.9999 | 0.99991 | 1 | 17915 | 0 | −3.2767 |
| PAQR4 | 4 | 9.94E−05 | 0.00036828 | 0.031953 | 206 | 4 | −2.9108 | 0.9999 | 0.99991 | 1 | 17914 | 0 | −2.9108 |
| EIF1AD | 4 | 9.95E−05 | 0.00036992 | 0.031953 | 207 | 4 | −2.4808 | 0.9999 | 0.99991 | 1 | 17913 | 0 | −2.4808 |
| ZFP64 | 4 | 0.00010169 | 0.00037705 | 0.03226 | 208 | 4 | −1.1375 | 0.9999 | 0.9999 | 1 | 17912 | 0 | −1.1375 |
| LSM12 | 4 | 0.00010186 | 0.00037705 | 0.03226 | 209 | 4 | −1.8783 | 0.9999 | 0.9999 | 1 | 17911 | 0 | −1.8783 |
| MVD | 4 | 0.0001031 | 0.00038254 | 0.032575 | 210 | 3 | −3.2853 | 0.80915 | 0.83692 | 1 | 13638 | 1 | −3.2853 |
| SOGA1 | 4 | 0.00010399 | 0.00038528 | 0.032655 | 211 | 4 | −2.054 | 0.9999 | 0.9999 | 1 | 17910 | 0 | −2.054 |
| GNB1L | 3 | 0.00010482 | 0.00032989 | 0.030486 | 212 | 3 | −1.3216 | 0.9999 | 0.99989 | 1 | 17909 | 0 | −1.3216 |
| PDCD5 | 4 | 0.0001054 | 0.00038967 | 0.032811 | 213 | 4 | −1.6523 | 0.99989 | 0.9999 | 1 | 17908 | 0 | −1.6523 |
| SUGT1 | 4 | 0.00010551 | 0.00039076 | 0.032811 | 214 | 4 | −3.2738 | 0.99989 | 0.9999 | 1 | 17907 | 0 | −3.2738 |
| TIMM10 | 4 | 0.00010587 | 0.00039405 | 0.032859 | 215 | 4 | −2.8579 | 0.99979 | 0.9998 | 1 | 17866 | 0 | −2.8579 |
| TRAPPC3 | 4 | 0.00010632 | 0.00039625 | 0.032859 | 216 | 4 | −1.889 | 0.99135 | 0.99146 | 1 | 17098 | 0 | −1.889 |
| HIGD2A | 4 | 0.00010646 | 0.0003968 | 0.032859 | 217 | 4 | −2.1526 | 0.99989 | 0.9999 | 1 | 17906 | 0 | −2.1526 |
| RNASEH1 | 4 | 0.00010948 | 0.00040447 | 0.033342 | 218 | 3 | −1.4536 | 0.86741 | 0.87386 | 1 | 14314 | 1 | −1.4536 |
| PRDX1 | 3 | 0.00010989 | 0.00034634 | 0.031262 | 219 | 3 | −5.1416 | 0.99989 | 0.99989 | 1 | 17902 | 0 | −5.1416 |
| NELFA | 4 | 0.00010999 | 0.00040667 | 0.033371 | 220 | 4 | −2.4818 | 0.99989 | 0.99989 | 1 | 17901 | 0 | −2.4818 |
| COX17 | 4 | 0.00011158 | 0.00041215 | 0.033668 | 221 | 4 | −1.4543 | 0.99989 | 0.99989 | 1 | 17900 | 0 | −1.4543 |
| SCAND1 | 4 | 0.00011312 | 0.00042093 | 0.034209 | 222 | 4 | −2.2206 | 0.99989 | 0.99989 | 1 | 17899 | 0 | −2.2206 |
| RPUSD4 | 4 | 0.00011361 | 0.00042257 | 0.034209 | 223 | 4 | −1.7369 | 0.98468 | 0.98475 | 1 | 16800 | 0 | −1.7369 |
| DIS3 | 4 | 0.0001147 | 0.00043025 | 0.034437 | 224 | 3 | −3.7289 | 0.93184 | 0.93154 | 1 | 15447 | 0 | −3.7289 |
| BRD2 | 4 | 0.00011561 | 0.00043409 | 0.034437 | 225 | 4 | −1.3229 | 0.99988 | 0.99989 | 1 | 17898 | 0 | −1.3229 |
| ARHGAP30 | 4 | 0.00011587 | 0.00043464 | 0.034437 | 226 | 4 | −1.5163 | 0.99988 | 0.99989 | 1 | 17897 | 0 | −1.5163 |
| PDSS2 | 4 | 0.00011599 | 0.00043519 | 0.034437 | 227 | 4 | −1.3758 | 0.99988 | 0.99989 | 1 | 17896 | 0 | −1.3758 |
| IPO9 | 4 | 0.00011605 | 0.00043574 | 0.034437 | 228 | 3 | −2 | 0.5322 | 0.68235 | 1 | 11233 | 1 | −2 |
| MRPS6 | 4 | 0.00011627 | 0.00043683 | 0.034437 | 229 | 4 | −1.5459 | 0.96886 | 0.96876 | 1 | 16307 | 0 | −1.5459 |
| CHMP7 | 4 | 0.00011903 | 0.00045054 | 0.035364 | 230 | 3 | −1.2879 | 0.95216 | 0.95203 | 1 | 15892 | 0 | −1.2879 |
| UGP2 | 4 | 0.0001208 | 0.00045932 | 0.035896 | 231 | 4 | −1.3438 | 0.99988 | 0.99988 | 1 | 17895 | 0 | −1.3438 |
| NHEJ1 | 4 | 0.00012205 | 0.00046425 | 0.036126 | 232 | 4 | −2.5344 | 0.99786 | 0.9979 | 1 | 17555 | 0 | −2.5344 |
| XRN2 | 4 | 0.00012329 | 0.00046645 | 0.036141 | 233 | 4 | −2.0988 | 0.99988 | 0.99988 | 1 | 17893 | 0 | −2.0988 |
| SHOC2 | 4 | 0.00012632 | 0.00048071 | 0.037086 | 234 | 3 | −1.6705 | 0.090242 | 0.20947 | 0.861805 | 4339 | 1 | −1.6705 |
| CPSF4 | 4 | 0.00013291 | 0.00051087 | 0.039246 | 235 | 4 | −1.5634 | 0.99824 | 0.99828 | 1 | 17605 | 0 | −1.5634 |
| ACTR1B | 4 | 0.00013826 | 0.00052678 | 0.04012 | 236 | 4 | −3.0796 | 0.99986 | 0.99987 | 1 | 17892 | 0 | −3.0796 |
| IBA57 | 4 | 0.00013868 | 0.00052787 | 0.04012 | 237 | 4 | −1.4957 | 0.99881 | 0.9988 | 1 | 17675 | 0 | −1.4957 |
| NDUFS2 | 4 | 0.0001392 | 0.00053007 | 0.04012 | 238 | 4 | −2.1487 | 0.99986 | 0.99987 | 1 | 17890 | 0 | −2.1487 |
| ACTR2 | 4 | 0.00013956 | 0.00053171 | 0.04012 | 239 | 4 | −3.5208 | 0.99986 | 0.99987 | 1 | 17889 | 0 | −3.5208 |
| KDSR | 4 | 0.00014013 | 0.00053336 | 0.04012 | 240 | 3 | −1.4275 | 0.78476 | 0.82391 | 1 | 13446 | 1 | −1.4275 |
| PRPS2 | 4 | 0.00014118 | 0.0005372 | 0.040241 | 241 | 4 | −1.9012 | 0.9987 | 0.9987 | 1 | 17656 | 0 | −1.9012 |
| TXNL4B | 4 | 0.00014243 | 0.00054378 | 0.040565 | 242 | 3 | −3.0725 | 0.92339 | 0.92323 | 1 | 15275 | 0 | −3.0725 |
| FGF10 | 4 | 0.00014344 | 0.00054707 | 0.040643 | 243 | 4 | −1.605 | 0.99986 | 0.99987 | 1 | 17887 | 0 | −1.605 |
| NSMCE4A | 4 | 0.00014588 | 0.00055639 | 0.041166 | 244 | 4 | −1.1608 | 0.96155 | 0.96142 | 1 | 16119 | 0 | −1.1608 |
| LRIF1 | 4 | 0.00015889 | 0.00060356 | 0.044376 | 245 | 4 | −1.9116 | 0.9988 | 0.99879 | 1 | 17671 | 0 | −1.9116 |
| C17orf70 | 4 | 0.00016044 | 0.00060904 | 0.044376 | 246 | 4 | −1.6102 | 0.99976 | 0.99977 | 1 | 17847 | 0 | −1.6102 |
| G6PD | 4 | 0.00016122 | 0.00061124 | 0.044376 | 247 | 4 | −2.906 | 0.99844 | 0.99847 | 1 | 17631 | 0 | −2.906 |
| TFRC | 4 | 0.00016142 | 0.00061124 | 0.044376 | 248 | 4 | −1.5981 | 0.98226 | 0.98233 | 1 | 16709 | 0 | −1.5981 |
| NELFCD | 4 | 0.00016201 | 0.00061343 | 0.044376 | 249 | 4 | −1.8838 | 0.99908 | 0.99907 | 1 | 17706 | 0 | −1.8838 |
| DARS2 | 4 | 0.0001622 | 0.00061453 | 0.044376 | 250 | 4 | −2.1492 | 0.98955 | 0.98966 | 1 | 17013 | 0 | −2.1492 |
| CLTC | 4 | 0.00016474 | 0.0006255 | 0.044828 | 251 | 4 | −1.529 | 0.99984 | 0.99985 | 1 | 17886 | 0 | −1.529 |
| MPI | 4 | 0.00016523 | 0.00062659 | 0.044828 | 252 | 4 | −1.4141 | 0.99983 | 0.99985 | 1 | 17885 | 0 | −1.4141 |
| RSBN1 | 4 | 0.00016581 | 0.00062824 | 0.044828 | 253 | 4 | −1.4498 | 0.99983 | 0.99985 | 1 | 17884 | 0 | −1.4498 |
| RPE | 4 | 0.00016696 | 0.00063208 | 0.044925 | 254 | 3 | −2.6854 | 0.65067 | 0.76739 | 1 | 12550 | 1 | −2.6854 |
| TADA1 | 4 | 0.0001693 | 0.00064195 | 0.045226 | 255 | 4 | −1.1451 | 0.99983 | 0.99984 | 1 | 17883 | 0 | −1.1451 |
| MAD2L2 | 4 | 0.00017039 | 0.00064469 | 0.045226 | 256 | 4 | −2.1551 | 0.99983 | 0.99984 | 1 | 17882 | 0 | −2.1551 |
| VPS16 | 4 | 0.00017098 | 0.00064579 | 0.045226 | 257 | 4 | −2.2913 | 0.99983 | 0.99984 | 1 | 17881 | 0 | −2.2913 |
| AMPD2 | 4 | 0.00017115 | 0.00064634 | 0.045226 | 258 | 4 | −0.94962 | 0.99983 | 0.99984 | 1 | 17880 | 0 | −0.94962 |
| LYNX1 | 4 | 0.00017259 | 0.00065237 | 0.045449 | 259 | 4 | −1.3465 | 0.99983 | 0.99984 | 1 | 17879 | 0 | −1.3465 |
| CABLES2 | 4 | 0.00017468 | 0.00065895 | 0.045579 | 260 | 3 | −1.7047 | 0.42934 | 0.59734 | 1 | 10025 | 1 | −1.7047 |
| SPATA5L1 | 4 | 0.00017545 | 0.00066169 | 0.045594 | 261 | 4 | −3.2615 | 0.99464 | 0.99474 | 1 | 17289 | 0 | −3.2615 |
| MRPL53 | 4 | 0.00017634 | 0.00066444 | 0.045609 | 262 | 4 | −2.2715 | 0.98057 | 0.98065 | 1 | 16649 | 0 | −2.2715 |
| NRF1 | 4 | 0.00018095 | 0.00068034 | 0.046348 | 263 | 4 | −1.3472 | 0.97087 | 0.9708 | 1 | 16359 | 0 | −1.3472 |
| ARMC7 | 4 | 0.00018347 | 0.00069131 | 0.046918 | 264 | 4 | −1.702 | 0.99982 | 0.99982 | 1 | 17878 | 0 | −1.702 |
| COG8 | 4 | 0.0001852 | 0.00069844 | 0.047224 | 265 | 4 | −1.7198 | 0.99355 | 0.99365 | 1 | 17227 | 0 | −1.7198 |
| SRC | 4 | 0.00018696 | 0.00070392 | 0.047418 | 266 | 4 | −1.772 | 0.99981 | 0.99982 | 1 | 17877 | 0 | −1.772 |
| DHPS | 4 | 0.00018909 | 0.00070996 | 0.047616 | 267 | 4 | −2.4307 | 0.99876 | 0.99875 | 1 | 17667 | 0 | −2.4307 |
| SAMM50 | 4 | 0.00018974 | 0.00071215 | 0.047616 | 268 | 4 | −1.8682 | 0.99761 | 0.99763 | 1 | 17527 | 0 | −1.8682 |
| NARS | 4 | 0.00019559 | 0.00073573 | 0.048349 | 269 | 4 | −2.9742 | 0.9998 | 0.99981 | 1 | 17876 | 0 | −2.9742 |
| WDR3 | 4 | 0.00019606 | 0.00073793 | 0.048349 | 270 | 4 | −1.8549 | 0.9998 | 0.99981 | 1 | 17875 | 0 | −1.8549 |
| PMPCA | 4 | 0.00019634 | 0.00074012 | 0.048349 | 271 | 4 | −4.1455 | 0.9998 | 0.99981 | 1 | 17874 | 0 | −4.1455 |
| ZNF408 | 4 | 0.00019644 | 0.00074067 | 0.048349 | 272 | 4 | −2.4366 | 0.9998 | 0.99981 | 1 | 17873 | 0 | −2.4366 |
| WDR18 | 4 | 0.00019759 | 0.00074286 | 0.048349 | 273 | 3 | −1.0326 | 0.68686 | 0.78075 | 1 | 12754 | 1 | −1.0326 |
| CPSF1 | 4 | 0.00019766 | 0.00074286 | 0.048349 | 274 | 4 | −2.5838 | 0.9998 | 0.99981 | 1 | 17872 | 0 | −2.5838 |
| CSTF1 | 4 | 0.00019791 | 0.00074341 | 0.048349 | 275 | 4 | −1.8878 | 0.99976 | 0.99976 | 1 | 17846 | 0 | −1.8878 |
| WARS2 | 4 | 0.00019822 | 0.00074506 | 0.048349 | 276 | 4 | −1.3629 | 0.9998 | 0.99981 | 1 | 17871 | 0 | −1.3629 |
| RPP40 | 4 | 0.00019988 | 0.00075081 | 0.048349 | 277 | 4 | −3.0167 | 0.99737 | 0.9974 | 1 | 17503 | 0 | −3.0167 |
| GRWD1 | 4 | 0.00020039 | 0.00075273 | 0.048349 | 278 | 3 | −1.8876 | 0.70339 | 0.78722 | 1 | 12854 | 1 | −1.8876 |
| EXOSC2 | 4 | 0.00020097 | 0.00075328 | 0.048349 | 279 | 4 | −3.2078 | 0.9998 | 0.99981 | 1 | 17870 | 0 | −3.2078 |
| TRAPPC1 | 4 | 0.00020378 | 0.0007637 | 0.048349 | 280 | 4 | −1.6652 | 0.99181 | 0.99194 | 1 | 17125 | 0 | −1.6652 |
| NAA10 | 4 | 0.00020385 | 0.00076425 | 0.048349 | 281 | 4 | −3.0089 | 0.9998 | 0.99981 | 1 | 17869 | 0 | −3.0089 |
| VAV3 | 4 | 0.00020476 | 0.00076699 | 0.048349 | 282 | 4 | −1.9808 | 0.99977 | 0.99977 | 1 | 17851 | 0 | −1.9808 |
| KRR1 | 4 | 0.00020517 | 0.00076754 | 0.048349 | 283 | 3 | −2.0954 | 0.93343 | 0.93319 | 1 | 15481 | 0 | −2.0954 |
| DKC1 | 4 | 0.00020529 | 0.00076864 | 0.048349 | 284 | 4 | −2.0448 | 0.99979 | 0.99981 | 1 | 17868 | 0 | −2.0448 |
| FBL | 4 | 0.00020558 | 0.00076864 | 0.048349 | 285 | 4 | −3.264 | 0.99979 | 0.99981 | 1 | 17867 | 0 | −3.264 |
| CDS2 | 4 | 0.00020672 | 0.00077193 | 0.048388 | 286 | 2 | −0.88413 | 0.6155 | 0.75335 | 1 | 12331 | 2 | −0.88413 |
| CHTF18 | 4 | 0.00020932 | 0.00077961 | 0.048511 | 287 | 3 | −1.7401 | 0.81525 | 0.84033 | 1 | 13698 | 1 | −1.7401 |
| LEMD2 | 4 | 0.00021117 | 0.00078674 | 0.048511 | 288 | 4 | −2.1887 | 0.99979 | 0.9998 | 1 | 17865 | 0 | −2.1887 |
| EARS2 | 4 | 0.00021119 | 0.00078729 | 0.048511 | 289 | 3 | −2.3607 | 0.030003 | 0.091951 | 0.703388 | 2317 | 1 | −2.3607 |
| MARS2 | 4 | 0.00021156 | 0.00078729 | 0.048511 | 290 | 4 | −3.4443 | 0.99979 | 0.9998 | 1 | 17864 | 0 | −3.4443 |
| DSCC1 | 4 | 0.00021196 | 0.00078783 | 0.048511 | 291 | 4 | −1.9729 | 0.99979 | 0.9998 | 1 | 17863 | 0 | −1.9729 |
| WDR4 | 4 | 0.00021315 | 0.00079003 | 0.048511 | 292 | 4 | −2.5209 | 0.99979 | 0.99979 | 1 | 17862 | 0 | −2.5209 |
| GMPPB | 3 | 0.00021564 | 0.00065456 | 0.045449 | 293 | 3 | −3.8284 | 0.99978 | 0.99978 | 1 | 17861 | 0 | −3.8284 |
| GFPT1 | 4 | 0.0002167 | 0.00080264 | 0.049087 | 294 | 4 | −1.4844 | 0.99773 | 0.99776 | 1 | 17540 | 0 | −1.4844 |
| ERCC1 | 4 | 0.00021732 | 0.00080484 | 0.049087 | 295 | 4 | −2.7125 | 0.97256 | 0.97251 | 1 | 16407 | 0 | −2.7125 |
| MED8 | 4 | 0.00021807 | 0.00080813 | 0.049122 | 296 | 4 | −1.6386 | 0.99978 | 0.99979 | 1 | 17860 | 0 | −1.6386 |
| PXN | 3 | 0.00022293 | 0.00067705 | 0.046298 | 297 | 2 | −2.0743 | 0.82724 | 0.82741 | 1 | 13828 | 1 | −2.0743 |
| NSMCE2 | 4 | 0.00022567 | 0.00083665 | 0.050684 | 298 | 4 | −1.6594 | 0.99977 | 0.99978 | 1 | 17859 | 0 | −1.6594 |
| NOC2L | 4 | 0.00022765 | 0.00084487 | 0.051012 | 299 | 4 | −2.4592 | 0.99977 | 0.99978 | 1 | 17857 | 0 | −2.4592 |
| ASNA1 | 4 | 0.00022828 | 0.00084981 | 0.051035 | 300 | 4 | −1.7093 | 0.99977 | 0.99978 | 1 | 17856 | 0 | −1.7093 |
| CHD1 | 4 | 0.0002287 | 0.0008509 | 0.051035 | 301 | 4 | −1.1862 | 0.99977 | 0.99978 | 1 | 17855 | 0 | −1.1862 |
| EXOSC3 | 4 | 0.00023038 | 0.00085749 | 0.051259 | 302 | 4 | −3.2232 | 0.99977 | 0.99977 | 1 | 17854 | 0 | −3.2232 |
| ATP6V1D | 4 | 0.0002341 | 0.0008701 | 0.051841 | 303 | 4 | −2.2986 | 0.99977 | 0.99977 | 1 | 17852 | 0 | −2.2986 |
| SPATA5 | 4 | 0.00023618 | 0.00087613 | 0.052021 | 304 | 4 | −2.0607 | 0.95647 | 0.95635 | 1 | 15985 | 0 | −2.0607 |
| LIN52 | 4 | 0.00023732 | 0.00087888 | 0.052021 | 305 | 4 | −1.7247 | 0.99976 | 0.99977 | 1 | 17849 | 0 | −1.7247 |
| MYCT1 | 4 | 0.00023873 | 0.00088436 | 0.052174 | 306 | 4 | −1.5124 | 0.99976 | 0.99977 | 1 | 17848 | 0 | −1.5124 |
| PTDSS1 | 4 | 0.00024176 | 0.00089204 | 0.052456 | 307 | 4 | −2.0389 | 0.99036 | 0.99044 | 1 | 17043 | 0 | −2.0389 |
| AHCY | 4 | 0.00024433 | 0.00089917 | 0.052565 | 308 | 3 | −2.2013 | 0.88136 | 0.88438 | 1 | 14513 | 1 | −2.2013 |
| ZBTB5 | 4 | 0.00024459 | 0.00089972 | 0.052565 | 309 | 4 | −1.8434 | 0.96229 | 0.96218 | 1 | 16135 | 0 | −1.8434 |
| RPTOR | 4 | 0.00024886 | 0.00091178 | 0.053098 | 310 | 4 | −1.1772 | 0.99975 | 0.99976 | 1 | 17845 | 0 | −1.1772 |
| DNAJC17 | 4 | 0.00025066 | 0.00091891 | 0.053341 | 311 | 4 | −2.2352 | 0.99975 | 0.99975 | 1 | 17844 | 0 | −2.2352 |
| DCLRE1B | 4 | 0.00025223 | 0.00092549 | 0.053551 | 312 | 4 | −2.2146 | 0.99975 | 0.99975 | 1 | 17843 | 0 | −2.2146 |
| SPCS3 | 4 | 0.00025484 | 0.00093427 | 0.053886 | 313 | 4 | −3.0664 | 0.99975 | 0.99975 | 1 | 17842 | 0 | −3.0664 |
| CCDC51 | 4 | 0.00025956 | 0.00095237 | 0.054644 | 314 | 3 | −3.1867 | 0.82841 | 0.8479 | 1 | 13841 | 1 | −3.1867 |
| WDR7 | 4 | 0.00026006 | 0.00095346 | 0.054644 | 315 | 4 | −2.2194 | 0.99631 | 0.99638 | 1 | 17412 | 0 | −2.2194 |
| RFWD3 | 4 | 0.00026326 | 0.00096608 | 0.055192 | 316 | 4 | −1.5941 | 0.99974 | 0.99974 | 1 | 17841 | 0 | −1.5941 |
| FANCE | 4 | 0.00026548 | 0.0009754 | 0.055549 | 317 | 4 | −0.93285 | 0.99973 | 0.99974 | 1 | 17840 | 0 | −0.93285 |
| VMP1 | 4 | 0.00026795 | 0.00098253 | 0.055779 | 318 | 4 | −2.9274 | 0.99806 | 0.99809 | 1 | 17579 | 0 | −2.9274 |
| MYBBP1A | 4 | 0.00027015 | 0.00099076 | 0.055936 | 319 | 3 | −1.7457 | 0.60167 | 0.74131 | 1 | 12139 | 1 | −1.7457 |
| MRPS34 | 4 | 0.00027057 | 0.0009924 | 0.055936 | 320 | 4 | −2.2369 | 0.99973 | 0.99973 | 1 | 17839 | 0 | −2.2369 |
| REG4 | 4 | 0.00027152 | 0.0009946 | 0.055936 | 321 | 4 | −1.3532 | 0.99973 | 0.99973 | 1 | 17838 | 0 | −1.3532 |
| TSSC1 | 4 | 0.00027428 | 0.0010056 | 0.056295 | 322 | 4 | −1.3482 | 0.99973 | 0.99973 | 1 | 17837 | 0 | −1.3482 |
| TAF3 | 4 | 0.00027457 | 0.0010072 | 0.056295 | 323 | 4 | −1.3523 | 0.99717 | 0.9972 | 1 | 17482 | 0 | −1.3523 |
| TRMU | 4 | 0.00028145 | 0.0010286 | 0.057313 | 324 | 4 | −1.098 | 0.99972 | 0.99973 | 1 | 17836 | 0 | −1.098 |
| GTF3C1 | 4 | 0.00028899 | 0.0010588 | 0.058812 | 325 | 4 | −3.1865 | 0.99791 | 0.99794 | 1 | 17559 | 0 | −3.1865 |
| SS18L1 | 4 | 0.0002942 | 0.0010774 | 0.059664 | 326 | 3 | −1.4778 | 0.89374 | 0.89474 | 1 | 14710 | 1 | −1.4778 |
| DNTTIP1 | 4 | 0.00030748 | 0.0011169 | 0.061662 | 327 | 4 | −2.0364 | 0.99969 | 0.9997 | 1 | 17831 | 0 | −2.0364 |
| EXOSC7 | 4 | 0.00031008 | 0.001124 | 0.06866 | 328 | 4 | −2.9929 | 0.99969 | 0.99971 | 1 | 17833 | 0 | −2.9929 |
| PAN3 | 4 | 0.00031154 | 0.0011301 | 0.062009 | 329 | 4 | −1.4735 | 0.99969 | 0.9997 | 1 | 17832 | 0 | −1.4735 |
| RBBP8 | 4 | 0.00031442 | 0.0011421 | 0.062412 | 330 | 4 | −1.4814 | 0.98424 | 0.98431 | 1 | 16787 | 0 | −1.4814 |
| PELP1 | 4 | 0.00031473 | 0.0011443 | 0.062412 | 331 | 3 | −3.0793 | 0.36084 | 0.54112 | 1 | 9242 | 1 | −3.0793 |
| NSMCE1 | 4 | 0.00031839 | 0.0011575 | 0.06294 | 332 | 4 | −2.5729 | 0.99877 | 0.99876 | 1 | 17668 | 0 | −2.5729 |
| ITGB1 | 4 | 0.00032068 | 0.0011641 | 0.063008 | 333 | 4 | −1.281 | 0.98249 | 0.98256 | 1 | 16717 | 0 | −1.281 |
| TIPRL | 4 | 0.00032131 | 0.0011657 | 0.063008 | 334 | 4 | −2.0985 | 0.99968 | 0.9997 | 1 | 17830 | 0 | −2.0985 |
| VPS35 | 4 | 0.00032332 | 0.0011695 | 0.063026 | 335 | 4 | −2.3848 | 0.99117 | 0.99129 | 1 | 17084 | 0 | −2.3848 |
| MRGBP | 4 | 0.00032554 | 0.0011778 | 0.063082 | 336 | 4 | −2.1045 | 0.99967 | 0.99969 | 1 | 17829 | 0 | −2.1045 |
| AGK | 4 | 0.00032674 | 0.0011805 | 0.063082 | 337 | 3 | −1.6248 | 0.77996 | 0.82149 | 1 | 13404 | 1 | −1.6248 |
| WDR61 | 4 | 0.00032705 | 0.0011811 | 0.063082 | 338 | 4 | −1.7425 | 0.99781 | 0.99784 | 1 | 17550 | 0 | −1.7425 |
| PGP | 4 | 0.00033428 | 0.0012101 | 0.064444 | 339 | 4 | −1.413 | 0.97033 | 0.97025 | 1 | 16343 | 0 | −1.413 |
| ZC3H18 | 4 | 0.00033676 | 0.0012173 | 0.064633 | 340 | 4 | −1.6472 | 0.99966 | 0.99968 | 1 | 17828 | 0 | −1.6472 |
| SLC31A1 | 4 | 0.00034032 | 0.0012293 | 0.064659 | 341 | 4 | −1.2026 | 0.99837 | 0.9984 | 1 | 17621 | 0 | −1.2026 |
| YARS | 4 | 0.0003417 | 0.0012315 | 0.064659 | 342 | 4 | −3.4562 | 0.99966 | 0.99968 | 1 | 17827 | 0 | −3.4562 |
| C20orf173 | 4 | 0.00034184 | 0.0012315 | 0.064659 | 343 | 4 | −1.3168 | 0.99966 | 0.99968 | 1 | 17826 | 0 | −1.3168 |
| TMCO6 | 4 | 0.00034193 | 0.0012321 | 0.064659 | 344 | 3 | −1.3293 | 0.74203 | 0.80358 | 1 | 13109 | 1 | −1.3293 |
| UBE2J2 | 4 | 0.00034341 | 0.0012425 | 0.065017 | 345 | 4 | −0.93031 | 0.99966 | 0.99968 | 1 | 17825 | 0 | −0.93031 |
| KCND3 | 4 | 0.00034694 | 0.0012518 | 0.065315 | 346 | 3 | −0.8166 | 0.94767 | 0.94758 | 1 | 15794 | 0 | −0.8166 |
| TOMM40 | 4 | 0.00034839 | 0.0012595 | 0.065316 | 347 | 4 | −2.1029 | 0.99098 | 0.99109 | 1 | 17070 | 0 | −2.1029 |
| ALDH1B1 | 4 | 0.00034914 | 0.0012617 | 0.065316 | 348 | 4 | −1.6365 | 0.99965 | 0.99967 | 1 | 17824 | 0 | −1.6365 |
| CTNNBL1 | 4 | 0.00035035 | 0.0012633 | 0.065316 | 349 | 4 | −1.5478 | 0.98214 | 0.98221 | 1 | 16704 | 0 | −1.5478 |
| CUL2 | 4 | 0.00035232 | 0.0012694 | 0.065316 | 350 | 4 | −1.5347 | 0.99965 | 0.99966 | 1 | 17823 | 0 | −1.5347 |
| MMP9 | 4 | 0.00035247 | 0.0012699 | 0.065316 | 351 | 4 | −1.5391 | 0.99965 | 0.99966 | 1 | 17822 | 0 | −1.5391 |
| MRPL4 | 4 | 0.00035722 | 0.0012919 | 0.066255 | 352 | 4 | −1.5598 | 0.99772 | 0.99774 | 1 | 17538 | 0 | −1.5598 |
| FBXW11 | 4 | 0.00035923 | 0.0012962 | 0.066292 | 353 | 4 | −2.2355 | 0.9964 | 0.99647 | 1 | 17420 | 0 | −2.2355 |
| CTDNEP1 | 4 | 0.00036097 | 0.0013028 | 0.06644 | 354 | 4 | −3.1234 | 0.99964 | 0.99965 | 1 | 17820 | 0 | −3.1234 |
| TMX2 | 4 | 0.00036274 | 0.0013132 | 0.066783 | 355 | 4 | −2.8509 | 0.99964 | 0.99965 | 1 | 17819 | 0 | −2.8509 |
| HDAC8 | 4 | 0.00036408 | 0.0013215 | 0.067012 | 356 | 4 | −2.9996 | 0.99964 | 0.99965 | 1 | 17818 | 0 | −2.9996 |
| PAK2 | 4 | 0.00036658 | 0.0013341 | 0.067329 | 357 | 4 | −1.3973 | 0.99942 | 0.99944 | 1 | 17772 | 0 | −1.3973 |
| SPATS1 | 4 | 0.00036677 | 0.0013352 | 0.067329 | 358 | 4 | −1.6701 | 0.99963 | 0.99965 | 1 | 17817 | 0 | −1.6701 |
| PSMG1 | 4 | 0.00037203 | 0.0013527 | 0.068024 | 359 | 4 | −1.5998 | 0.99963 | 0.99964 | 1 | 17816 | 0 | −1.5998 |
| CCDC101 | 4 | 0.0003734 | 0.0013566 | 0.068028 | 360 | 4 | −1.5931 | 0.99963 | 0.99964 | 1 | 17815 | 0 | −1.5931 |
| EXOSC4 | 4 | 0.00039224 | 0.0014185 | 0.070798 | 361 | 4 | −2.9209 | 0.99961 | 0.99962 | 1 | 17813 | 0 | −2.9209 |
| C14orf166 | 4 | 0.00039238 | 0.0014196 | 0.070798 | 362 | 3 | −1.1611 | 0.95084 | 0.95075 | 1 | 15858 | 0 | −1.1611 |
| NOP14 | 4 | 0.00039477 | 0.0014279 | 0.071007 | 363 | 4 | −1.7104 | 0.99961 | 0.99962 | 1 | 17812 | 0 | −1.7104 |
| RPF2 | 4 | 0.00039588 | 0.0014317 | 0.071007 | 364 | 4 | −2.3747 | 0.9996 | 0.99962 | 1 | 17811 | 0 | −2.3747 |
| NAF1 | 4 | 0.00039842 | 0.0014372 | 0.071084 | 365 | 4 | −0.90399 | 0.97274 | 0.9727 | 1 | 16415 | 0 | −0.90399 |
| TIMM50 | 4 | 0.00040195 | 0.0014498 | 0.071473 | 366 | 4 | −2.5624 | 0.99626 | 0.99634 | 1 | 17408 | 0 | −2.5624 |
| CHCHD4 | 4 | 0.00040338 | 0.0014547 | 0.071473 | 367 | 4 | −1.8702 | 0.9996 | 0.99962 | 1 | 17810 | 0 | −1.8702 |
| LRRC66 | 4 | 0.00040402 | 0.0014569 | 0.071473 | 368 | 4 | −1.0465 | 0.9996 | 0.99961 | 1 | 17809 | 0 | −1.0465 |
| TM2D3 | 4 | 0.00040528 | 0.0014635 | 0.071601 | 369 | 4 | −1.0648 | 0.98695 | 0.98703 | 1 | 16900 | 0 | −1.0648 |
| COASY | 4 | 0.00040644 | 0.0014701 | 0.071729 | 370 | 4 | −2.6917 | 0.99959 | 0.99961 | 1 | 17808 | 0 | −2.6917 |
| PTCD3 | 4 | 0.00040741 | 0.0014745 | 0.071749 | 371 | 4 | −2.8325 | 0.99959 | 0.99961 | 1 | 17807 | 0 | −2.8325 |
| SDHC | 4 | 0.0004135 | 0.0014953 | 0.072479 | 372 | 4 | −1.077 | 0.98882 | 0.98895 | 1 | 16978 | 0 | −1.077 |
| TUBD1 | 4 | 0.00041409 | 0.0014975 | 0.072479 | 373 | 4 | −1.0269 | 0.99959 | 0.9996 | 1 | 17806 | 0 | −1.0269 |
| LRWD1 | 4 | 0.00042035 | 0.0015173 | 0.073023 | 374 | 4 | −0.9837 | 0.99958 | 0.9996 | 1 | 17804 | 0 | −0.9837 |
| DNAJC9 | 4 | 0.00042229 | 0.0015233 | 0.073023 | 375 | 4 | −2.1842 | 0.99447 | 0.99458 | 1 | 17280 | 0 | −2.1842 |
| FAM229B | 4 | 0.00042337 | 0.0015244 | 0.073023 | 376 | 4 | −1.1439 | 0.96358 | 0.96348 | 1 | 16174 | 0 | −1.1439 |
| IPO11 | 4 | 0.00042384 | 0.0015249 | 0.073023 | 377 | 4 | −2.6061 | 0.99958 | 0.9996 | 1 | 17803 | 0 | −2.6061 |
| HSPA13 | 4 | 0.00042823 | 0.0015397 | 0.073426 | 378 | 4 | −1.0463 | 0.99883 | 0.99882 | 1 | 17678 | 0 | −1.0463 |
| EXOC3 | 4 | 0.00042954 | 0.001543 | 0.073426 | 379 | 4 | −1.6023 | 0.99957 | 0.99959 | 1 | 17802 | 0 | −1.6023 |
| RPUSD3 | 4 | 0.00043157 | 0.0015491 | 0.073426 | 380 | 4 | −1.2902 | 0.99957 | 0.99959 | 1 | 17801 | 0 | −1.2902 |
| SAMD10 | 4 | 0.0004319 | 0.0015496 | 0.073426 | 381 | 4 | −1.4334 | 0.99957 | 0.99959 | 1 | 17800 | 0 | −1.4334 |
| CD3EAP | 4 | 0.00044406 | 0.0015946 | 0.075353 | 382 | 4 | −1.361 | 0.99893 | 0.99893 | 1 | 17690 | 0 | −1.361 |
| PPIH | 4 | 0.00044559 | 0.0016006 | 0.075353 | 383 | 4 | −1.8339 | 0.9994 | 0.99942 | 1 | 17764 | 0 | −1.8339 |
| C19orf40 | 4 | 0.00044612 | 0.0016028 | 0.075353 | 384 | 4 | −1.659 | 0.99955 | 0.99957 | 1 | 17799 | 0 | −1.659 |
| HUWE1 | 4 | 0.00045204 | 0.0016313 | 0.076395 | 385 | 4 | −1.4621 | 0.99955 | 0.99957 | 1 | 17797 | 0 | −1.4621 |
| DPH5 | 4 | 0.00045406 | 0.0016374 | 0.076395 | 386 | 4 | −1.5933 | 0.99815 | 0.99819 | 1 | 17593 | 0 | −1.5933 |
| EPAS1 | 4 | 0.00045485 | 0.0016393 | 0.076395 | 387 | 4 | −1.2677 | 0.99955 | 0.99957 | 1 | 17796 | 0 | −1.2677 |
| SEMA4A | 4 | 0.0004559 | 0.0016439 | 0.076395 | 388 | 4 | −1.7125 | 0.99954 | 0.99957 | 1 | 17795 | 0 | −1.7125 |
| SV2A | 4 | 0.00045643 | 0.0016461 | 0.076395 | 389 | 4 | −1.3699 | 0.99954 | 0.99957 | 1 | 17794 | 0 | −1.3699 |
| PREB | 4 | 0.00045873 | 0.0016544 | 0.07641 | 390 | 4 | −3.8362 | 0.99954 | 0.99957 | 1 | 17793 | 0 | −3.8362 |
| CRCP | 4 | 0.00045911 | 0.0016549 | 0.07641 | 391 | 4 | −2.0486 | 0.99647 | 0.99652 | 1 | 17426 | 0 | −2.0486 |
| RAD1 | 4 | 0.0004638 | 0.0016714 | 0.076973 | 392 | 4 | −2.8802 | 0.9983 | 0.99832 | 1 | 17614 | 0 | −2.8802 |
| URM1 | 4 | 0.00047254 | 0.0017065 | 0.078366 | 393 | 3 | −1.2973 | 0.95106 | 0.95096 | 1 | 15863 | 0 | −1.2973 |
| NOP56 | 4 | 0.00047341 | 0.0017103 | 0.078366 | 394 | 4 | −2.4596 | 0.99953 | 0.99955 | 1 | 17791 | 0 | −2.4596 |
| PAICS | 4 | 0.00047488 | 0.0017174 | 0.078445 | 395 | 4 | −2.2487 | 0.99417 | 0.99429 | 1 | 17264 | 0 | −2.2487 |
| GMEB2 | 4 | 0.00048371 | 0.0017465 | 0.07942 | 396 | 3 | −1.3261 | 0.60755 | 0.74638 | 1 | 12217 | 1 | −1.3261 |
| PDAP1 | 4 | 0.00048863 | 0.001758 | 0.079743 | 397 | 4 | −1.9559 | 0.99951 | 0.99953 | 1 | 17789 | 0 | −1.9559 |
| C19orf52 | 4 | 0.00048979 | 0.0017657 | 0.07989 | 398 | 4 | −1.7897 | 0.96095 | 0.96083 | 1 | 16105 | 0 | −1.7897 |
| RPP14 | 4 | 0.00049142 | 0.0017717 | 0.079963 | 399 | 3 | −1.2774 | 0.14502 | 0.29801 | 0.935696 | 5699 | 1 | −1.2774 |
| PRSS33 | 4 | 0.00049273 | 0.0017783 | 0.08006 | 400 | 4 | −0.77872 | 0.99951 | 0.99953 | 1 | 17788 | 0 | −0.77872 |
| SLC33A1 | 4 | 0.00049779 | 0.0017975 | 0.080491 | 401 | 4 | −1.0499 | 0.9995 | 0.99952 | 1 | 17787 | 0 | −1.0499 |
| STRIP1 | 4 | 0.00049838 | 0.0018003 | 0.080491 | 402 | 4 | −2.0981 | 0.99801 | 0.99805 | 1 | 17573 | 0 | −2.0981 |
| EMC6 | 4 | 0.00049964 | 0.0018024 | 0.080491 | 403 | 4 | −1.3197 | 0.97029 | 0.9702 | 1 | 16342 | 0 | −1.3197 |
| LSG1 | 4 | 0.00050086 | 0.0018057 | 0.080491 | 404 | 4 | −1.776 | 0.97262 | 0.97257 | 1 | 16409 | 0 | −1.776 |
| THG1L | 4 | 0.0005021 | 0.0018118 | 0.080561 | 405 | 4 | −2.6563 | 0.99394 | 0.99405 | 1 | 17246 | 0 | −2.6563 |
| WDR25 | 4 | 0.00050334 | 0.0018167 | 0.080582 | 406 | 3 | −0.97109 | 0.89526 | 0.89611 | 1 | 14733 | 1 | −0.97109 |
| INTS8 | 4 | 0.00051494 | 0.0018584 | 0.082052 | 407 | 4 | −1.4338 | 0.99949 | 0.99951 | 1 | 17786 | 0 | −1.4338 |
| MPDU1 | 4 | 0.00051503 | 0.0018589 | 0.082052 | 408 | 3 | −1.2897 | 0.95251 | 0.95237 | 1 | 15898 | 0 | −1.2897 |
| ARF1 | 4 | 0.00051671 | 0.001865 | 0.082118 | 409 | 4 | −1.4624 | 0.95998 | 0.95986 | 1 | 16080 | 0 | −1.4624 |
| POLRMT | 4 | 0.00052075 | 0.0018792 | 0.082368 | 410 | 4 | −1.1245 | 0.99948 | 0.9995 | 1 | 17785 | 0 | −1.1245 |
| SYS1 | 4 | 0.00052095 | 0.0018798 | 0.082368 | 411 | 4 | −1.225 | 0.99948 | 0.9995 | 1 | 17784 | 0 | −1.225 |
| EXOSC6 | 4 | 0.00052661 | 0.0018995 | 0.083032 | 412 | 4 | −1.6302 | 0.99947 | 0.9995 | 1 | 17783 | 0 | −1.6302 |
| SEC61B | 4 | 0.00052907 | 0.0019072 | 0.083166 | 413 | 3 | −2.1515 | 0.94648 | 0.94638 | 1 | 15767 | 0 | −2.1515 |
| ARHGDIA | 4 | 0.00053213 | 0.001916 | 0.083171 | 414 | 4 | −1.3426 | 0.99947 | 0.99949 | 1 | 17782 | 0 | −1.3426 |
| DOLPP1 | 4 | 0.00053221 | 0.0019165 | 0.083171 | 415 | 3 | −1.5424 | 0.82559 | 0.8462 | 1 | 13808 | 1 | −1.5424 |
| TELO2 | 4 | 0.0005398 | 0.0019374 | 0.083873 | 416 | 4 | −1.9296 | 0.99843 | 0.99846 | 1 | 17630 | 0 | −1.9296 |
| ATP5F1 | 4 | 0.0005443 | 0.0019549 | 0.084431 | 417 | 4 | −2.4241 | 0.99946 | 0.99948 | 1 | 17781 | 0 | −2.4241 |
| NOL12 | 4 | 0.00054551 | 0.0019648 | 0.084655 | 418 | 4 | −1.1313 | 0.99945 | 0.99947 | 1 | 17780 | 0 | −1.1313 |
| NUDC | 4 | 0.00054955 | 0.0019785 | 0.084907 | 419 | 4 | −2.4637 | 0.99945 | 0.99947 | 1 | 17779 | 0 | −2.4637 |
| SRF | 4 | 0.00055178 | 0.0019878 | 0.084907 | 420 | 4 | −2.3237 | 0.99945 | 0.99947 | 1 | 17778 | 0 | −2.3237 |
| ATP6V1B2 | 4 | 0.00055219 | 0.0019889 | 0.084907 | 421 | 4 | −1.6378 | 0.99945 | 0.99947 | 1 | 17777 | 0 | −1.6378 |
| UBA5 | 4 | 0.0005526 | 0.0019895 | 0.084907 | 422 | 4 | −0.93046 | 0.99945 | 0.99947 | 1 | 17776 | 0 | −0.93046 |
| USF2 | 4 | 0.0005575 | 0.0020163 | 0.085851 | 423 | 4 | −0.9727 | 0.99944 | 0.99946 | 1 | 17775 | 0 | −0.9727 |
| ATP6V1E1 | 4 | 0.00056222 | 0.0020355 | 0.086332 | 424 | 4 | −1.648 | 0.99944 | 0.99946 | 1 | 17773 | 0 | −1.648 |
| WRAP53 | 4 | 0.00056267 | 0.0020372 | 0.086332 | 425 | 4 | −2.0119 | 0.99924 | 0.99924 | 1 | 17737 | 0 | −2.0119 |
| COX11 | 4 | 0.0005658 | 0.0020525 | 0.086779 | 426 | 4 | −1.9149 | 0.99383 | 0.99392 | 1 | 17241 | 0 | −1.9149 |
| MALT1 | 4 | 0.00057978 | 0.0021052 | 0.088752 | 427 | 3 | −1.4036 | 0.92754 | 0.92727 | 1 | 15359 | 0 | −1.4036 |
| SSR4 | 4 | 0.00058101 | 0.0021096 | 0.088752 | 428 | 4 | −1.1029 | 0.99942 | 0.99944 | 1 | 17771 | 0 | −1.1029 |
| LARS | 4 | 0.00058249 | 0.002114 | 0.088752 | 429 | 4 | −1.7444 | 0.99942 | 0.99944 | 1 | 17770 | 0 | −1.7444 |
| COMMD3 | 3 | 0.00058298 | 0.0017207 | 0.078445 | 430 | 3 | −1.6111 | 0.99942 | 0.99943 | 1 | 17769 | 0 | −1.6111 |
| PGM3 | 4 | 0.00058571 | 0.0021238 | 0.08896 | 431 | 4 | −1.1952 | 0.99788 | 0.99792 | 1 | 17556 | 0 | −1.1952 |
| LAS1L | 4 | 0.00058822 | 0.0021321 | 0.089097 | 432 | 4 | −1.6342 | 0.99941 | 0.99943 | 1 | 17768 | 0 | −1.6342 |
| MGAT1 | 4 | 0.00059269 | 0.0021463 | 0.089189 | 433 | 4 | −1.7315 | 0.98788 | 0.98797 | 1 | 16942 | 0 | −1.7315 |
| UCKL1 | 4 | 0.00059272 | 0.0021463 | 0.089189 | 434 | 4 | −1.4633 | 0.99941 | 0.99943 | 1 | 17767 | 0 | −1.4633 |
| DDX51 | 4 | 0.00059314 | 0.0021491 | 0.089189 | 435 | 4 | −4.1583 | 0.99941 | 0.99943 | 1 | 17766 | 0 | −4.1583 |
| INTS6 | 4 | 0.00059702 | 0.0021617 | 0.089483 | 436 | 4 | −2.182 | 0.9994 | 0.99942 | 1 | 17765 | 0 | −2.182 |
| GRB2 | 4 | 0.00060002 | 0.0021721 | 0.089527 | 437 | 4 | −2.4286 | 0.99868 | 0.9987 | 1 | 17654 | 0 | −2.4286 |
| RPP38 | 4 | 0.00060374 | 0.0021891 | 0.090022 | 438 | 4 | −1.9113 | 0.9989 | 0.99889 | 1 | 17686 | 0 | −1.9113 |
| PPP2R3C | 4 | 0.00060678 | 0.0022028 | 0.090355 | 439 | 4 | −1.602 | 0.99939 | 0.99941 | 1 | 17763 | 0 | −1.602 |
| KTI12 | 4 | 0.00060878 | 0.0022072 | 0.090355 | 440 | 4 | −2.466 | 0.99521 | 0.99529 | 1 | 17341 | 0 | −2.466 |
| RNMTL1 | 4 | 0.00061219 | 0.0022198 | 0.090452 | 441 | 3 | −1.8789 | 0.90059 | 0.90082 | 1 | 14819 | 1 | −1.8789 |
| IL6ST | 4 | 0.00061313 | 0.0022275 | 0.090452 | 442 | 4 | −1.0172 | 0.99939 | 0.9994 | 1 | 17762 | 0 | −1.0172 |
| GNB2 | 4 | 0.00061379 | 0.0022297 | 0.090452 | 443 | 4 | −1.1386 | 0.99939 | 0.9994 | 1 | 17761 | 0 | −1.1386 |
| VPS45 | 4 | 0.00061468 | 0.0022335 | 0.090452 | 444 | 4 | −1.2876 | 0.99939 | 0.9994 | 1 | 17760 | 0 | −1.2876 |
| FRMPD1 | 4 | 0.00061549 | 0.0022346 | 0.090452 | 445 | 4 | −1.7098 | 0.99874 | 0.99873 | 1 | 17662 | 0 | −1.7098 |
| GTF2H3 | 4 | 0.00061722 | 0.0022434 | 0.090604 | 446 | 4 | −1.4963 | 0.99924 | 0.99924 | 1 | 17736 | 0 | −1.4963 |
| TPM3 | 4 | 0.00062261 | 0.0022593 | 0.091043 | 447 | 4 | −2.0802 | 0.9985 | 0.99852 | 1 | 17640 | 0 | −2.0802 |
| CASS4 | 4 | 0.00062644 | 0.0022714 | 0.091325 | 448 | 4 | −1.0339 | 0.99937 | 0.99939 | 1 | 17759 | 0 | −1.0339 |
| NDST1 | 4 | 0.00063272 | 0.0022922 | 0.091824 | 449 | 4 | −0.98521 | 0.99937 | 0.99939 | 1 | 17758 | 0 | −0.98521 |
| OIP5 | 4 | 0.00063411 | 0.0022977 | 0.091824 | 450 | 3 | −3.4598 | 0.075267 | 0.184 | 0.838039 | 3918 | 1 | −3.4598 |
| BRK1 | 4 | 0.00063508 | 0.0023021 | 0.091824 | 451 | 3 | −1.3128 | 0.63025 | 0.76037 | 1 | 12431 | 1 | −1.3128 |
| PPME1 | 4 | 0.00063633 | 0.0023059 | 0.091824 | 452 | 4 | −1.284 | 0.99936 | 0.99938 | 1 | 17757 | 0 | −1.284 |
| FASTKD5 | 4 | 0.00063701 | 0.0023092 | 0.091824 | 453 | 4 | −1.1004 | 0.99767 | 0.99769 | 1 | 17531 | 0 | −1.1004 |
| POLR1E | 4 | 0.0006452 | 0.0023399 | 0.092034 | 454 | 4 | −1.8714 | 0.99935 | 0.99937 | 1 | 17756 | 0 | −1.8714 |
| LRP6 | 4 | 0.00064527 | 0.0023399 | 0.092034 | 455 | 4 | −1.3315 | 0.9984 | 0.99842 | 1 | 17624 | 0 | −1.3315 |
| TSEN34 | 4 | 0.00064636 | 0.0023421 | 0.092034 | 456 | 3 | −1.7605 | 0.13757 | 0.28637 | 0.926506 | 5519 | 1 | −1.7605 |
| OR6N1 | 4 | 0.00064673 | 0.0023443 | 0.092034 | 457 | 4 | −1.3652 | 0.98089 | 0.98096 | 1 | 16663 | 0 | −1.3652 |
| MRPL21 | 4 | 0.0006468 | 0.0023443 | 0.092034 | 458 | 4 | −2.2199 | 0.99935 | 0.99937 | 1 | 17755 | 0 | −2.2199 |
| CLTA | 4 | 0.00064818 | 0.0023525 | 0.092034 | 459 | 4 | −1.1949 | 0.99935 | 0.99937 | 1 | 17754 | 0 | −1.1949 |
| FXN | 4 | 0.00064864 | 0.0023536 | 0.092034 | 460 | 4 | −2.6112 | 0.99935 | 0.99937 | 1 | 17753 | 0 | −2.6112 |
| PAGR1 | 4 | 0.00064912 | 0.0023553 | 0.092034 | 461 | 4 | −1.9271 | 0.9851 | 0.98518 | 1 | 16818 | 0 | −1.9271 |
| EXOSC10 | 4 | 0.00065506 | 0.0023767 | 0.092669 | 462 | 4 | −1.3546 | 0.996 | 0.99609 | 1 | 17387 | 0 | −1.3546 |
| FDX1L | 4 | 0.0006568 | 0.0023843 | 0.092768 | 463 | 3 | −1.4012 | 0.78762 | 0.8254 | 1 | 13476 | 1 | −1.4012 |
| BPTF | 4 | 0.00065855 | 0.0023909 | 0.092779 | 464 | 4 | −1.6284 | 0.96998 | 0.9699 | 1 | 16334 | 0 | −1.6284 |
| TRMT5 | 4 | 0.00066051 | 0.0024008 | 0.092779 | 465 | 3 | −3.1085 | 0.67524 | 0.77631 | 1 | 12693 | 1 | −3.1085 |
| CCT4 | 4 | 0.00066158 | 0.0024046 | 0.092779 | 466 | 4 | −2.4854 | 0.99934 | 0.99935 | 1 | 17752 | 0 | −2.4854 |
| SNRNP70 | 4 | 0.00066182 | 0.0024052 | 0.092779 | 467 | 4 | −1.7606 | 0.99934 | 0.99935 | 1 | 17751 | 0 | −1.7606 |
| ZNRD1 | 4 | 0.00066381 | 0.0024107 | 0.092793 | 468 | 3 | −2.0479 | 0.72482 | 0.79609 | 1 | 12993 | 1 | −2.0479 |
| OPN5 | 4 | 0.00066557 | 0.0024189 | 0.092911 | 469 | 3 | −1.1585 | 0.80879 | 0.83672 | 1 | 13636 | 1 | −1.1585 |
| TXN2 | 4 | 0.00066647 | 0.0024244 | 0.092924 | 470 | 3 | −2.2332 | 0.58001 | 0.72283 | 1 | 11870 | 1 | −2.2332 |
| NOL9 | 4 | 0.00066946 | 0.0024348 | 0.093126 | 471 | 3 | −1.9172 | 0.84789 | 0.86019 | 1 | 14070 | 1 | −1.9172 |
| GCK | 4 | 0.00067425 | 0.0024507 | 0.093536 | 472 | 4 | −1.0828 | 0.99933 | 0.99934 | 1 | 17750 | 0 | −1.0828 |
| RAD51D | 4 | 0.00067616 | 0.0024584 | 0.093631 | 473 | 3 | −2.0314 | 0.073456 | 0.18091 | 0.835346 | 3865 | 1 | −2.0314 |
| SEPSECS | 4 | 0.00068149 | 0.002482 | 0.093946 | 474 | 3 | −1.3233 | 0.3741 | 0.55194 | 1 | 9405 | 1 | −1.3233 |
| MOB4 | 4 | 0.00068203 | 0.0024842 | 0.093946 | 475 | 3 | −1.3514 | 0.88137 | 0.88439 | 1 | 14514 | 1 | −1.3514 |
| MIPEP | 4 | 0.0006828 | 0.0024877 | 0.093946 | 476 | 4 | −1.7921 | 0.99932 | 0.99933 | 1 | 17749 | 0 | −1.7921 |
| SPCS2 | 4 | 0.00068447 | 0.0024927 | 0.093946 | 477 | 4 | −2.0957 | 0.99932 | 0.99933 | 1 | 17748 | 0 | −2.0957 |
| PHF12 | 4 | 0.00068662 | 0.0025023 | 0.094111 | 478 | 4 | −1.5343 | 0.99931 | 0.99933 | 1 | 17747 | 0 | −1.5343 |
| NAA25 | 4 | 0.00068863 | 0.002511 | 0.094111 | 479 | 3 | −1.3876 | 0.77013 | 0.81673 | 1 | 13322 | 1 | −1.3876 |
| PSRC1 | 4 | 0.00069041 | 0.0025143 | 0.094111 | 480 | 3 | −0.80914 | 0.95266 | 0.95251 | 1 | 15903 | 0 | −0.80914 |
| SPAG4 | 4 | 0.00069169 | 0.0025204 | 0.094111 | 481 | 4 | −1.8811 | 0.99809 | 0.99813 | 1 | 17587 | 0 | −1.8811 |
| NAA35 | 4 | 0.00069263 | 0.0025231 | 0.094111 | 482 | 4 | −2.7694 | 0.99931 | 0.99932 | 1 | 17746 | 0 | −2.7694 |
| PTPMT1 | 4 | 0.0007011 | 0.0025516 | 0.094803 | 483 | 4 | −2.2622 | 0.9993 | 0.99931 | 1 | 17745 | 0 | −2.2622 |
| UBE3D | 4 | 0.0007012 | 0.0025522 | 0.094803 | 484 | 4 | −1.1363 | 0.99742 | 0.99746 | 1 | 17511 | 0 | −1.1363 |
| KCNA3 | 4 | 0.00070549 | 0.0025714 | 0.09532 | 485 | 4 | −1.4333 | 0.99929 | 0.99931 | 1 | 17744 | 0 | −1.4333 |
| ELOF1 | 4 | 0.00071543 | 0.0026015 | 0.096084 | 486 | 4 | −2.4501 | 0.99226 | 0.99238 | 1 | 17148 | 0 | −2.4501 |
| TCEA2 | 4 | 0.00071631 | 0.0026026 | 0.096084 | 487 | 4 | −1.1414 | 0.99928 | 0.99929 | 1 | 17743 | 0 | −1.1414 |
| ADAT3 | 4 | 0.00072487 | 0.0026317 | 0.096959 | 488 | 4 | −1.7534 | 0.99768 | 0.9977 | 1 | 17532 | 0 | −1.7534 |
| PIM1 | 4 | 0.00072698 | 0.0026421 | 0.097145 | 489 | 3 | −1.5728 | 0.86989 | 0.87571 | 1 | 14351 | 1 | −1.5728 |
| SCO1 | 4 | 0.0007305 | 0.0026514 | 0.097289 | 490 | 4 | −1.4324 | 0.99927 | 0.99928 | 1 | 17742 | 0 | −1.4324 |
| PMPCB | 4 | 0.0007328 | 0.0026635 | 0.097534 | 491 | 4 | −1.5035 | 0.99559 | 0.99567 | 1 | 17361 | 0 | −1.5035 |
| DYRK1A | 4 | 0.00073604 | 0.0026756 | 0.097777 | 492 | 4 | −0.97407 | 0.99926 | 0.99927 | 1 | 17741 | 0 | −0.97407 |
| ORAOV1 | 4 | 0.00073983 | 0.0026876 | 0.09802 | 493 | 4 | −2.7829 | 0.99926 | 0.99926 | 1 | 17740 | 0 | −2.7829 |
| B3GALT6 | 3 | 0.00074084 | 0.0021661 | 0.089483 | 494 | 3 | −1.4899 | 0.9335 | 0.93366 | 1 | 15482 | 0 | −1.4899 |
| THUMPD1 | 4 | 0.00074796 | 0.002709 | 0.098601 | 495 | 4 | −1.0086 | 0.99925 | 0.99926 | 1 | 17739 | 0 | −1.0086 |
| GPN2 | 4 | 0.00075283 | 0.0027266 | 0.09904 | 496 | 4 | −4.257 | 0.99925 | 0.99925 | 1 | 17738 | 0 | −4.257 |
| TBL3 | 4 | 0.00075908 | 0.0027463 | 0.099557 | 497 | 4 | −1.6298 | 0.99882 | 0.99881 | 1 | 17677 | 0 | −1.6298 |
| BGLAP | 4 | 0.00076198 | 0.0027551 | 0.099675 | 498 | 4 | −0.73621 | 0.99307 | 0.99318 | 1 | 17194 | 0 | −0.73621 |
| ADAT2 | 3 | 0.00086003 | 0.0024924 | 0.093946 | 525 | 3 | −1.3692 | 0.99914 | 0.99915 | 1 | 17717 | 0 | −1.3692 |

HCC44 MRTX SL Gene FDR 0.1

| | | | | | | | | | | | | | |
|---|---|---|---|---|---|---|---|---|---|---|---|---|---|
| PDAP1 | 4 | 4.85E−09 | 2.74E−07 | 0.00165 | 1 | 4 | −1.4287 | 1 | 1 | 1 | 18053 | 0 | −1.4287 |
| KTI12 | 4 | 3.61E−08 | 2.74E−07 | 0.00165 | 2 | 3 | −1.0632 | 0.90505 | 0.90471 | 1 | 15789 | 0 | −1.0632 |
| ELP3 | 4 | 1.74E−07 | 2.74E−07 | 0.00165 | 3 | 4 | −1.0645 | 0.99982 | 0.99983 | 1 | 18021 | 0 | −1.0645 |
| ELP5 | 4 | 2.51E−07 | 8.23E−07 | 0.003713 | 4 | 4 | −1.4708 | 1 | 1 | 1 | 18049 | 0 | −1.4708 |
| ELP4 | 4 | 4.35E−07 | 1.37E−06 | 0.00495 | 5 | 4 | −0.94242 | 1 | 1 | 1 | 18052 | 0 | −0.94242 |
| PRPF38B | 4 | 6.62E−07 | 1.92E−06 | 0.005776 | 6 | 4 | −0.88846 | 1 | 1 | 1 | 18051 | 0 | −0.88846 |
| SLC39A9 | 4 | 9.49E−07 | 3.02E−06 | 0.007779 | 7 | 4 | −0.87878 | 0.99953 | 0.99955 | 1 | 18001 | 0 | −0.87878 |
| WRB | 4 | 1.32E−06 | 4.11E−06 | 0.009282 | 8 | 4 | −0.70872 | 1 | 1 | 1 | 18050 | 0 | −0.70872 |
| UTP23 | 4 | 2.47E−06 | 6.31E−06 | 0.012651 | 9 | 4 | −0.90621 | 0.99959 | 0.99961 | 1 | 18008 | 0 | −0.90621 |
| IRAK1 | 4 | 4.85E−06 | 1.67E−05 | 0.028353 | 10 | 4 | −0.55233 | 1 | 1 | 1 | 18048 | 0 | −0.55233 |
| MOB4 | 4 | 5.26E−06 | 1.73E−05 | 0.028353 | 11 | 4 | −0.85116 | 0.99727 | 0.9973 | 1 | 17897 | 0 | −0.85116 |
| MRPS24 | 4 | 6.20E−06 | 2.00E−05 | 0.030084 | 12 | 4 | −0.72907 | 0.99999 | 1 | 1 | 18047 | 0 | −0.72907 |
| TELO2 | 4 | 6.92E−06 | 2.17E−05 | 0.030084 | 13 | 4 | −0.67696 | 0.99988 | 0.99988 | 1 | 18026 | 0 | −0.67696 |
| EXT2 | 4 | 8.73E−06 | 2.93E−05 | 0.036634 | 14 | 3 | −0.787 | 0.78761 | 0.79528 | 1 | 13684 | 1 | −0.787 |
| FNDC3B | 4 | 8.92E−06 | 3.04E−05 | 0.036634 | 15 | 3 | −0.71452 | 0.76152 | 0.77611 | 1 | 13333 | 1 | −0.71452 |
| GNB1 | 4 | 1.09E−05 | 3.59E−05 | 0.040532 | 16 | 4 | −0.74453 | 0.99969 | 0.99971 | 1 | 18013 | 0 | −0.74453 |
| CRK | 4 | 1.22E−05 | 4.41E−05 | 0.046884 | 17 | 3 | −0.88303 | 0.95684 | 0.95673 | 1 | 16880 | 0 | −0.88303 |
| URM1 | 4 | 1.73E−05 | 6.44E−05 | 0.061751 | 18 | 4 | −0.76168 | 0.99306 | 0.99318 | 1 | 17744 | 0 | −0.76168 |
| EXT1 | 4 | 1.74E−05 | 6.50E−05 | 0.061751 | 19 | 4 | −0.72429 | 0.99787 | 0.99791 | 1 | 17920 | 0 | −0.72429 |
| TOE1 | 4 | 2.00E−05 | 7.38E−05 | 0.066584 | 20 | 4 | −0.4679 | 0.98872 | 0.98886 | 1 | 17616 | 0 | −0.4679 |
| SNUPN | 4 | 2.12E−05 | 7.82E−05 | 0.066939 | 21 | 4 | −0.75892 | 0.99998 | 0.99998 | 1 | 18046 | 0 | −0.75892 |
| PMAIP1 | 4 | 2.22E−05 | 8.20E−05 | 0.066939 | 22 | 4 | −0.5658 | 0.99997 | 0.99997 | 1 | 18043 | 0 | −0.5658 |
| RIOK2 | 4 | 2.28E−05 | 8.53E−05 | 0.066939 | 23 | 4 | −0.56299 | 0.99998 | 0.99998 | 1 | 18045 | 0 | −0.56299 |
| NHP2 | 4 | 2.56E−05 | 9.41E−05 | 0.070751 | 24 | 4 | −0.91413 | 0.99997 | 0.99997 | 1 | 18044 | 0 | −0.91413 |
| BRF2 | 4 | 2.82E−05 | 0.00010338 | 0.074653 | 25 | 4 | −0.66669 | 0.99592 | 0.99601 | 1 | 17846 | 0 | −0.66669 |
| LSM10 | 4 | 3.30E−05 | 0.00011874 | 0.079368 | 26 | 3 | −0.64553 | 0.84639 | 0.84582 | 1 | 14627 | 1 | −0.64553 |
| XYLT2 | 4 | 3.38E−05 | 0.00012148 | 0.079368 | 27 | 4 | −0.69258 | 0.98486 | 0.98493 | 1 | 17519 | 0 | −0.69258 |
| CDIPT | 4 | 3.48E−05 | 0.00012751 | 0.079368 | 28 | 4 | −0.98317 | 0.99997 | 0.99997 | 1 | 18042 | 0 | −0.98317 |
| WWTR1 | 4 | 3.64E−05 | 0.000133 | 0.079368 | 29 | 4 | −0.82829 | 0.99996 | 0.99996 | 1 | 18041 | 0 | −0.82829 |
| SSSCA1 | 4 | 3.64E−05 | 0.000133 | 0.079368 | 30 | 4 | −0.63072 | 0.99996 | 0.99996 | 1 | 18040 | 0 | −0.63072 |
| PKN2 | 4 | 3.71E−05 | 0.00013629 | 0.079368 | 31 | 4 | −0.71003 | 0.99996 | 0.99996 | 1 | 18039 | 0 | −0.71003 |
| PUM1 | 4 | 4.05E−05 | 0.0001511 | 0.081101 | 32 | 3 | −0.81704 | 0.96108 | 0.96095 | 1 | 16978 | 0 | −0.81704 |
| C11orf57 | 4 | 4.08E−05 | 0.00015219 | 0.081101 | 33 | 4 | −0.53508 | 0.99996 | 0.99996 | 1 | 18038 | 0 | −0.53508 |
| GEMIN7 | 4 | 4.10E−05 | 0.00015274 | 0.081101 | 34 | 4 | −0.59475 | 0.99614 | 0.99622 | 1 | 17854 | 0 | −0.59475 |
| BAIAP3 | 4 | 4.38E−05 | 0.00016206 | 0.082921 | 35 | 3 | −0.61836 | 0.94895 | 0.94885 | 1 | 16707 | 0 | −0.61836 |
| ZBTB17 | 4 | 4.50E−05 | 0.00016535 | 0.082921 | 36 | 3 | −0.66464 | 0.96196 | 0.96183 | 1 | 17000 | 0 | −0.66464 |
| RPL14 | 4 | 4.66E−05 | 0.00017084 | 0.083356 | 37 | 4 | −0.88701 | 0.98859 | 0.98871 | 1 | 17613 | 0 | −0.88701 |
| SART3 | 4 | 4.95E−05 | 0.00018345 | 0.086189 | 38 | 4 | −0.70653 | 0.99995 | 0.99995 | 1 | 18037 | 0 | −0.70653 |
| FANCA | 4 | 5.00E−05 | 0.0001862 | 0.086189 | 39 | 4 | −0.52457 | 0.99995 | 0.99995 | 1 | 18036 | 0 | −0.52457 |
| IKBKB | 4 | 5.52E−05 | 0.0002032 | 0.091708 | 40 | 3 | −0.47975 | 0.96507 | 0.96498 | 1 | 17068 | 0 | −0.47975 |
| COPS6 | 4 | 5.70E−05 | 0.00021033 | 0.09261 | 41 | 4 | −0.72607 | 0.99994 | 0.99995 | 1 | 18035 | 0 | −0.72607 |

Shared and unique synthetic lethal (SL) genes were identified in the four lines (FIG. 1C; Table 2). More than 40 genes were depleted (FDR<0.1) in at least three lines (FIG. 1D); even more (˜334) “dropped out” in at least two (Table 2). Heterogeneity at the gene level probably reflects other genetic/epigenetic changes in these lines (in addition to KRAS, STK11, and KEAP1). SHOC2, an known SL gene in MEK-inhibitor treated cells (21), was depleted in 3/4 lines, as were genes encoding several enzymes that might be targeted therapeutically, including three serine/threonine kinases (VRK1, RIOK2, PK1N), multiple components of the elongator complex (ELP2, ELP3, ELP4, ELP5), metabolic genes (PGD, PGM3), and genes (EXT1, EXT2) involved in heparan sulfate biosynthesis, among others. Enrichr analysis revealed SL pathways (p<0.05) shared in at least two lines, with most common to three or four (FIG. 1E). Several of these (colored red) were expected based on knowledge of RAS pathway action and inhibitor effects (22,23), including MAPK Family Signaling, PI3K/AKT/mTOR Signaling, FGF Signaling, PDGF Signaling, Autophagy, C-MYC Pathway and E2F targets. Others represent potential novel, parallel targets (black). Genes belonging to the Hippo (YAP/TAZ) pathway were enriched in dropouts from 3/4 lines; in addition, TEAD1 and WWTR1 (encoding TAZ) were each SL in two lines, while YAP1 showed significant dropout in H2030 (55, 56) (FIGS. 1D and 1E; Table 2).

TABLE 2
Overlapping synthetic lethal (i.e., dropout genes) in at least
2 cell lines (FDR <0.1) from MRTX-849 (adagrasib) CRISPR/Cas9
screens in NSCLC cell lines. (True = synthetic lethal
in that line; False = not synthetic lethal in that line)

| | H2122- | H2030- | HCC44- | |
|---|---|---|---|---|
| id | MRTX | MRTX | MRTX | H23-MRTX |
| RTCB | TRUE | TRUE | FALSE | TRUE |
| RNASEH2A | TRUE | FALSE | FALSE | TRUE |
| LSM10 | TRUE | FALSE | TRUE | FALSE |
| JMJD6 | TRUE | FALSE | FALSE | TRUE |
| PTPMT1 | TRUE | TRUE | FALSE | TRUE |
| RCL1 | TRUE | FALSE | FALSE | TRUE |
| PGD | TRUE | TRUE | FALSE | TRUE |
| ALG1 | TRUE | FALSE | FALSE | TRUE |
| DOLK | TRUE | FALSE | FALSE | TRUE |
| MRPL53 | TRUE | FALSE | FALSE | TRUE |
| WRB | TRUE | FALSE | TRUE | TRUE |
| C7orf55-LUC7L2 | TRUE | FALSE | FALSE | TRUE |
| UTP23 | TRUE | FALSE | TRUE | TRUE |
| KDM2A | TRUE | FALSE | FALSE | TRUE |
| ATP6V1F | TRUE | FALSE | FALSE | TRUE |
| ENO1 | TRUE | FALSE | FALSE | TRUE |
| SLC7A5 | TRUE | FALSE | FALSE | TRUE |
| SLC33A1 | TRUE | FALSE | FALSE | TRUE |
| DKC1 | TRUE | FALSE | FALSE | TRUE |
| GEMIN7 | TRUE | TRUE | TRUE | TRUE |
| NAE1 | TRUE | FALSE | FALSE | TRUE |
| RPE | TRUE | FALSE | FALSE | TRUE |
| GNB1L | TRUE | FALSE | FALSE | TRUE |
| TEN1 | TRUE | TRUE | FALSE | TRUE |
| SDHB | TRUE | FALSE | FALSE | TRUE |
| MTG2 | TRUE | FALSE | FALSE | TRUE |
| TRAPPC3 | TRUE | FALSE | FALSE | TRUE |
| ELP5 | TRUE | TRUE | TRUE | TRUE |
| MAD2L2 | TRUE | FALSE | FALSE | TRUE |
| PTCD3 | TRUE | FALSE | FALSE | TRUE |
| CSTF1 | TRUE | FALSE | FALSE | TRUE |
| LIN52 | TRUE | FALSE | FALSE | TRUE |
| DOHH | TRUE | FALSE | FALSE | TRUE |
| MRPL4 | TRUE | FALSE | FALSE | TRUE |
| DDX59 | TRUE | FALSE | FALSE | TRUE |
| DBR1 | TRUE | FALSE | FALSE | TRUE |
| RABGGTB | TRUE | FALSE | FALSE | TRUE |
| WARS2 | TRUE | FALSE | FALSE | TRUE |
| N6AMT1 | TRUE | FALSE | FALSE | TRUE |
| NSMCE1 | TRUE | FALSE | FALSE | TRUE |
| OTUD5 | TRUE | FALSE | FALSE | TRUE |
| URB1 | TRUE | FALSE | FALSE | TRUE |
| ARL2 | TRUE | FALSE | FALSE | TRUE |
| FDXR | TRUE | FALSE | FALSE | TRUE |
| DNAJC9 | TRUE | FALSE | FALSE | TRUE |
| AHCY | TRUE | FALSE | FALSE | TRUE |
| ERCC2 | TRUE | FALSE | FALSE | TRUE |
| SAE1 | TRUE | FALSE | FALSE | TRUE |
| MRPS6 | TRUE | FALSE | FALSE | TRUE |
| PHF12 | TRUE | FALSE | FALSE | TRUE |
| GMPPB | TRUE | TRUE | FALSE | TRUE |
| FDX1L | TRUE | FALSE | FALSE | TRUE |
| NAA25 | TRUE | FALSE | FALSE | TRUE |
| FARS2 | TRUE | FALSE | FALSE | TRUE |
| MARS2 | TRUE | FALSE | FALSE | TRUE |
| HSD17B10 | TRUE | FALSE | FALSE | TRUE |
| RNMT | TRUE | FALSE | FALSE | TRUE |
| TSSC1 | TRUE | FALSE | FALSE | TRUE |
| PPP6C | TRUE | FALSE | FALSE | TRUE |
| MTOR | TRUE | FALSE | FALSE | TRUE |
| DPAGT1 | TRUE | FALSE | FALSE | TRUE |
| SEC63 | TRUE | TRUE | FALSE | TRUE |
| GAPDH | TRUE | FALSE | FALSE | TRUE |
| GNB2L1 | TRUE | FALSE | FALSE | TRUE |
| DHX33 | TRUE | FALSE | FALSE | TRUE |
| ELP4 | TRUE | FALSE | TRUE | TRUE |
| ANKRD49 | TRUE | TRUE | FALSE | TRUE |
| ALG2 | TRUE | FALSE | FALSE | TRUE |
| MTX1 | TRUE | TRUE | FALSE | FALSE |
| HUWE1 | TRUE | FALSE | FALSE | TRUE |
| NOC4L | TRUE | FALSE | FALSE | TRUE |
| EMC1 | TRUE | FALSE | FALSE | TRUE |
| VMA21 | TRUE | FALSE | FALSE | TRUE |
| TADA1 | TRUE | FALSE | FALSE | TRUE |
| PARS2 | TRUE | FALSE | FALSE | TRUE |
| HNF1B | TRUE | TRUE | FALSE | FALSE |
| CUL2 | TRUE | FALSE | FALSE | TRUE |
| TSEN54 | TRUE | FALSE | FALSE | TRUE |
| TRMT61A | TRUE | FALSE | FALSE | TRUE |
| IARS | TRUE | FALSE | FALSE | TRUE |
| MCL1 | TRUE | TRUE | FALSE | FALSE |
| TRIT1 | TRUE | TRUE | FALSE | TRUE |
| PTDSS1 | TRUE | TRUE | FALSE | TRUE |
| IBA57 | TRUE | FALSE | FALSE | TRUE |
| TSEN2 | TRUE | FALSE | FALSE | TRUE |
| RTEL1 | TRUE | FALSE | FALSE | TRUE |
| TRAPPC1 | TRUE | FALSE | FALSE | TRUE |
| WDR61 | TRUE | FALSE | FALSE | TRUE |
| FAM96B | TRUE | FALSE | FALSE | TRUE |
| STT3A | TRUE | TRUE | FALSE | TRUE |
| PGM3 | TRUE | TRUE | FALSE | TRUE |
| WDR7 | TRUE | FALSE | FALSE | TRUE |
| VPS29 | TRUE | TRUE | FALSE | FALSE |
| WDR25 | TRUE | FALSE | FALSE | TRUE |
| MOGS | TRUE | TRUE | FALSE | TRUE |
| CLTC | TRUE | FALSE | FALSE | TRUE |
| BUB3 | TRUE | FALSE | FALSE | TRUE |
| DDOST | TRUE | FALSE | FALSE | TRUE |
| CCNC | TRUE | FALSE | FALSE | TRUE |
| NELFB | TRUE | FALSE | FALSE | TRUE |
| PSMG4 | TRUE | FALSE | FALSE | TRUE |
| RPN1 | TRUE | TRUE | FALSE | TRUE |
| TEX10 | TRUE | FALSE | FALSE | TRUE |
| MRPS34 | TRUE | FALSE | FALSE | TRUE |
| DAP3 | TRUE | FALSE | FALSE | TRUE |
| TIMM10 | TRUE | FALSE | FALSE | TRUE |
| PPP2R4 | TRUE | FALSE | FALSE | TRUE |
| FKBPL | TRUE | TRUE | FALSE | FALSE |
| THG1L | TRUE | FALSE | FALSE | TRUE |
| SPATA5L1 | TRUE | FALSE | FALSE | TRUE |
| TBCB | TRUE | FALSE | FALSE | TRUE |
| POLR3K | TRUE | FALSE | FALSE | TRUE |
| EIF3F | TRUE | TRUE | FALSE | FALSE |
| ADAT3 | TRUE | FALSE | FALSE | TRUE |
| MMS19 | TRUE | TRUE | FALSE | FALSE |
| EARS2 | TRUE | FALSE | FALSE | TRUE |
| GUK1 | TRUE | FALSE | FALSE | TRUE |
| NHLRC2 | TRUE | FALSE | FALSE | TRUE |
| TAF1C | TRUE | FALSE | FALSE | TRUE |
| SHOC2 | TRUE | TRUE | FALSE | TRUE |
| AP2S1 | TRUE | FALSE | FALSE | TRUE |
| MRPL47 | TRUE | FALSE | FALSE | TRUE |
| TPI1 | TRUE | FALSE | FALSE | TRUE |
| HDAC3 | TRUE | TRUE | FALSE | FALSE |
| CINP | TRUE | FALSE | FALSE | TRUE |
| TCEB2 | TRUE | FALSE | FALSE | TRUE |
| NDNL2 | TRUE | FALSE | FALSE | TRUE |
| VPS45 | TRUE | FALSE | FALSE | TRUE |
| RPL14 | TRUE | FALSE | TRUE | FALSE |
| CD3EAP | TRUE | FALSE | FALSE | TRUE |
| AIFM1 | TRUE | FALSE | FALSE | TRUE |
| DNAJC17 | TRUE | FALSE | FALSE | TRUE |
| PSMG1 | TRUE | FALSE | FALSE | TRUE |
| POP5 | TRUE | FALSE | FALSE | TRUE |
| CPSF4 | TRUE | FALSE | FALSE | TRUE |
| NELFA | TRUE | FALSE | FALSE | TRUE |
| RPP21 | TRUE | FALSE | FALSE | TRUE |
| CHTF8 | TRUE | FALSE | FALSE | TRUE |
| ORAOV1 | TRUE | FALSE | FALSE | TRUE |
| VMP1 | TRUE | FALSE | FALSE | TRUE |
| PDAP1 | TRUE | FALSE | TRUE | TRUE |
| ELP3 | TRUE | TRUE | TRUE | TRUE |
| PDSS2 | TRUE | FALSE | FALSE | TRUE |
| MED8 | TRUE | FALSE | FALSE | TRUE |
| ACTR6 | TRUE | FALSE | FALSE | TRUE |
| ELP2 | TRUE | TRUE | FALSE | TRUE |
| TOE1 | TRUE | FALSE | TRUE | TRUE |
| CCT4 | TRUE | FALSE | FALSE | TRUE |
| OIP5 | TRUE | FALSE | FALSE | TRUE |
| UGP2 | TRUE | TRUE | FALSE | TRUE |
| MRPL21 | TRUE | FALSE | FALSE | TRUE |
| VHL | TRUE | FALSE | FALSE | TRUE |
| CENPN | TRUE | TRUE | FALSE | FALSE |
| WDR77 | TRUE | FALSE | FALSE | TRUE |
| MCMBP | TRUE | FALSE | FALSE | TRUE |
| OPA1 | TRUE | TRUE | FALSE | TRUE |
| RPTOR | TRUE | FALSE | FALSE | TRUE |
| GGPS1 | TRUE | FALSE | FALSE | TRUE |
| IL6ST | TRUE | FALSE | FALSE | TRUE |
| COASY | TRUE | FALSE | FALSE | TRUE |
| MRPL28 | TRUE | FALSE | FALSE | TRUE |
| PPP4C | TRUE | FALSE | FALSE | TRUE |
| DYRK1A | TRUE | FALSE | FALSE | TRUE |
| PAICS | TRUE | FALSE | FALSE | TRUE |
| EXT2 | TRUE | TRUE | TRUE | FALSE |
| TEAD1 | TRUE | TRUE | FALSE | FALSE |
| SDHC | TRUE | FALSE | FALSE | TRUE |
| GFPT1 | TRUE | FALSE | FALSE | TRUE |
| NARS | TRUE | FALSE | FALSE | TRUE |
| ATP6V1D | TRUE | FALSE | FALSE | TRUE |
| NOL9 | TRUE | FALSE | FALSE | TRUE |
| POLRMT | TRUE | FALSE | FALSE | TRUE |
| GRPEL1 | TRUE | TRUE | FALSE | FALSE |
| EXOSC4 | TRUE | FALSE | FALSE | TRUE |
| SPATA5 | TRUE | FALSE | FALSE | TRUE |
| ZNRD1 | TRUE | FALSE | FALSE | TRUE |
| IPO11 | TRUE | TRUE | FALSE | TRUE |
| NHP2 | TRUE | FALSE | TRUE | TRUE |
| NAA10 | TRUE | FALSE | FALSE | TRUE |
| MOCS3 | TRUE | TRUE | FALSE | FALSE |
| EIF2B5 | TRUE | TRUE | FALSE | FALSE |
| DTYMK | TRUE | FALSE | FALSE | TRUE |
| KRR1 | TRUE | FALSE | FALSE | TRUE |
| RIOK2 | TRUE | TRUE | TRUE | TRUE |
| RAD51D | TRUE | FALSE | FALSE | TRUE |
| MBTPS2 | TRUE | FALSE | FALSE | TRUE |
| B3GNT2 | TRUE | TRUE | FALSE | FALSE |
| PMPCA | TRUE | FALSE | FALSE | TRUE |
| CHCHD4 | TRUE | FALSE | FALSE | TRUE |
| HNRNPU | TRUE | FALSE | FALSE | TRUE |
| RAB10 | TRUE | TRUE | FALSE | FALSE |
| ADNP | TRUE | TRUE | FALSE | FALSE |
| FXN | TRUE | FALSE | FALSE | TRUE |
| ARPC4 | TRUE | FALSE | FALSE | TRUE |
| METTL3 | TRUE | FALSE | FALSE | TRUE |
| GPN2 | TRUE | FALSE | FALSE | TRUE |
| ARF1 | TRUE | FALSE | FALSE | TRUE |
| SRP9 | TRUE | FALSE | FALSE | TRUE |
| SAMM50 | TRUE | FALSE | FALSE | TRUE |
| EIF3H | TRUE | TRUE | FALSE | FALSE |
| ATP6V1B2 | TRUE | FALSE | FALSE | TRUE |
| DHPS | TRUE | FALSE | FALSE | TRUE |
| NUDC | TRUE | FALSE | FALSE | TRUE |
| POLR1E | TRUE | FALSE | FALSE | TRUE |
| ASNA1 | TRUE | FALSE | FALSE | TRUE |
| DNAJB11 | TRUE | TRUE | FALSE | FALSE |
| TBP | TRUE | TRUE | FALSE | TRUE |
| CNOT1 | TRUE | TRUE | FALSE | FALSE |
| ARMC5 | TRUE | FALSE | FALSE | TRUE |
| SRP14 | TRUE | FALSE | FALSE | TRUE |
| EIF3A | TRUE | TRUE | FALSE | FALSE |
| HYOU1 | TRUE | FALSE | FALSE | TRUE |
| THOC6 | TRUE | TRUE | FALSE | FALSE |
| BPTF | TRUE | FALSE | FALSE | TRUE |
| FNTB | TRUE | FALSE | FALSE | TRUE |
| C17orf70 | TRUE | TRUE | FALSE | TRUE |
| MRPS24 | TRUE | FALSE | TRUE | FALSE |
| RPP38 | TRUE | FALSE | FALSE | TRUE |
| PTBP1 | TRUE | TRUE | FALSE | FALSE |
| COPS6 | TRUE | FALSE | TRUE | FALSE |
| CDK7 | TRUE | FALSE | FALSE | TRUE |
| BCCIP | TRUE | FALSE | FALSE | TRUE |
| TXN2 | TRUE | FALSE | FALSE | TRUE |
| TFRC | TRUE | FALSE | FALSE | TRUE |
| CTNNBL1 | TRUE | FALSE | FALSE | TRUE |
| TIMM22 | TRUE | FALSE | FALSE | TRUE |
| ATP5F1 | TRUE | FALSE | FALSE | TRUE |
| C14orf80 | TRUE | FALSE | FALSE | TRUE |
| FBL | TRUE | FALSE | FALSE | TRUE |
| G6PD | TRUE | FALSE | FALSE | TRUE |
| CIRH1A | TRUE | TRUE | FALSE | FALSE |
| ERBB3 | TRUE | TRUE | FALSE | FALSE |
| ACTR3 | TRUE | TRUE | FALSE | TRUE |
| GRWD1 | TRUE | FALSE | FALSE | TRUE |
| NAA20 | TRUE | FALSE | FALSE | TRUE |
| WBSCR16 | TRUE | FALSE | FALSE | TRUE |
| EIF1AD | TRUE | FALSE | FALSE | TRUE |
| VRK1 | TRUE | TRUE | FALSE | TRUE |
| PAK1IP1 | TRUE | TRUE | FALSE | FALSE |
| GTF2H3 | TRUE | FALSE | FALSE | TRUE |
| ALG9 | TRUE | TRUE | FALSE | FALSE |
| ATP6V1E1 | TRUE | FALSE | FALSE | TRUE |
| DSCC1 | TRUE | FALSE | FALSE | TRUE |
| DIS3 | TRUE | FALSE | FALSE | TRUE |
| ASCC3 | TRUE | TRUE | FALSE | FALSE |
| STT3B | TRUE | TRUE | FALSE | FALSE |
| ILF2 | TRUE | TRUE | FALSE | FALSE |
| KTI12 | TRUE | TRUE | TRUE | TRUE |
| OGFR | TRUE | FALSE | FALSE | TRUE |
| TOMM40 | TRUE | FALSE | FALSE | TRUE |
| DDX6 | TRUE | TRUE | FALSE | FALSE |
| SPCS2 | TRUE | FALSE | FALSE | TRUE |
| GTF3C1 | TRUE | FALSE | FALSE | TRUE |
| GSG2 | TRUE | FALSE | FALSE | TRUE |
| DEXI | TRUE | TRUE | FALSE | FALSE |
| MVD | TRUE | FALSE | FALSE | TRUE |
| DCLRE1B | TRUE | FALSE | FALSE | TRUE |
| MTG1 | TRUE | FALSE | FALSE | TRUE |
| COX17 | TRUE | FALSE | FALSE | TRUE |
| CHMP7 | TRUE | FALSE | FALSE | TRUE |
| AASDHPPT | TRUE | FALSE | FALSE | TRUE |
| PDSS1 | TRUE | FALSE | FALSE | TRUE |
| DARS2 | TRUE | FALSE | FALSE | TRUE |
| GFM1 | TRUE | FALSE | FALSE | TRUE |
| PDCD6IP | TRUE | FALSE | FALSE | TRUE |
| NCAPD2 | TRUE | TRUE | FALSE | FALSE |
| DDX51 | TRUE | FALSE | FALSE | TRUE |
| EMC6 | TRUE | FALSE | FALSE | TRUE |
| EXOSC2 | TRUE | TRUE | FALSE | TRUE |
| TBCE | TRUE | FALSE | FALSE | TRUE |
| EXOSC10 | TRUE | FALSE | FALSE | TRUE |
| NDUFS2 | TRUE | FALSE | FALSE | TRUE |
| ARMC7 | TRUE | FALSE | FALSE | TRUE |
| IMP3 | TRUE | FALSE | FALSE | TRUE |
| RABIF | TRUE | TRUE | FALSE | FALSE |
| NOP56 | TRUE | FALSE | FALSE | TRUE |
| WDR18 | TRUE | FALSE | FALSE | TRUE |
| PMPCB | TRUE | FALSE | FALSE | TRUE |
| CENPO | TRUE | TRUE | FALSE | FALSE |
| MIPEP | TRUE | FALSE | FALSE | TRUE |
| FANCA | TRUE | FALSE | TRUE | FALSE |
| GRB2 | TRUE | FALSE | FALSE | TRUE |
| ALG13 | TRUE | FALSE | FALSE | TRUE |
| TAF2 | TRUE | FALSE | FALSE | TRUE |
| ORC3 | TRUE | FALSE | FALSE | TRUE |
| SOD2 | TRUE | FALSE | FALSE | TRUE |
| C14orf166 | TRUE | FALSE | FALSE | TRUE |
| CPSF1 | TRUE | FALSE | FALSE | TRUE |
| PRMT1 | TRUE | FALSE | FALSE | TRUE |
| MPI | TRUE | TRUE | FALSE | TRUE |
| NELFCD | TRUE | FALSE | FALSE | TRUE |
| SNUPN | TRUE | FALSE | TRUE | FALSE |
| MPDU1 | TRUE | FALSE | FALSE | TRUE |
| TMEM165 | TRUE | TRUE | FALSE | FALSE |
| ASUN | TRUE | FALSE | FALSE | TRUE |
| GMPS | TRUE | FALSE | FALSE | TRUE |
| CDIPT | TRUE | TRUE | TRUE | TRUE |
| INTS10 | TRUE | FALSE | FALSE | TRUE |
| YARS | TRUE | FALSE | FALSE | TRUE |
| TFB2M | TRUE | FALSE | FALSE | TRUE |
| PAK2 | TRUE | FALSE | FALSE | TRUE |
| ERCC1 | TRUE | FALSE | FALSE | TRUE |
| MRPS18A | TRUE | FALSE | FALSE | TRUE |
| ALG5 | TRUE | TRUE | FALSE | FALSE |
| COX11 | TRUE | FALSE | FALSE | TRUE |
| PDCD5 | TRUE | FALSE | FALSE | TRUE |
| AHCYL1 | TRUE | TRUE | FALSE | FALSE |
| NSMCE2 | TRUE | FALSE | FALSE | TRUE |
| EXOC2 | TRUE | FALSE | FALSE | TRUE |
| TYMS | TRUE | FALSE | FALSE | TRUE |
| MESDC2 | TRUE | TRUE | FALSE | FALSE |
| MIS18A | TRUE | TRUE | FALSE | FALSE |
| GGNBP2 | TRUE | TRUE | FALSE | FALSE |
| SLC31A1 | FALSE | TRUE | FALSE | TRUE |
| NDST1 | FALSE | TRUE | FALSE | TRUE |
| SLC39A9 | FALSE | TRUE | TRUE | FALSE |
| LEMD2 | FALSE | TRUE | FALSE | TRUE |
| EXT1 | FALSE | TRUE | TRUE | TRUE |
| IKBKAP | FALSE | TRUE | FALSE | TRUE |
| ILF3 | FALSE | TRUE | FALSE | TRUE |
| URM1 | FALSE | TRUE | TRUE | TRUE |
| ACTR2 | FALSE | TRUE | FALSE | TRUE |
| PKN2 | FALSE | TRUE | TRUE | TRUE |
| WWTR1 | FALSE | TRUE | TRUE | FALSE |
| SEPSECS | FALSE | TRUE | FALSE | TRUE |
| RIC8A | FALSE | TRUE | FALSE | TRUE |
| IPO9 | FALSE | TRUE | FALSE | TRUE |
| BRK1 | FALSE | TRUE | FALSE | TRUE |
| SRC | FALSE | TRUE | FALSE | TRUE |
| MOB4 | FALSE | FALSE | TRUE | TRUE |
| TELO2 | FALSE | FALSE | TRUE | TRUE |
| CRK | FALSE | FALSE | TRUE | TRUE |
| XYLT2 | FALSE | FALSE | TRUE | TRUE |
| C11orf57 | FALSE | FALSE | TRUE | TRUE |

### Example 2. YAP1/TAZ/TEAD Inhibition Enhances Adagrasib Action

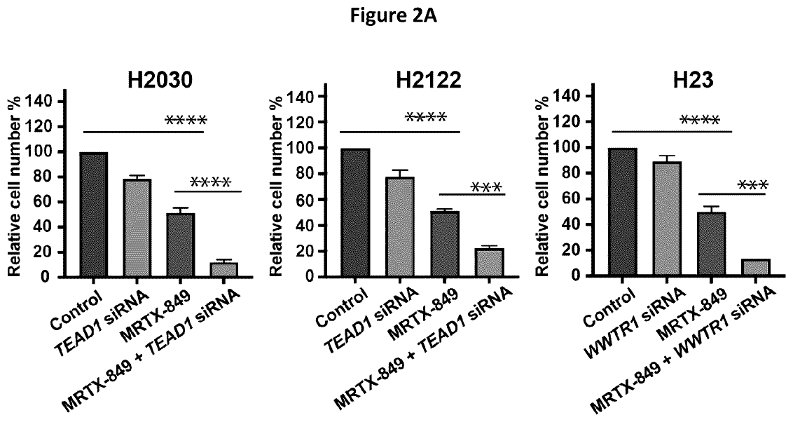

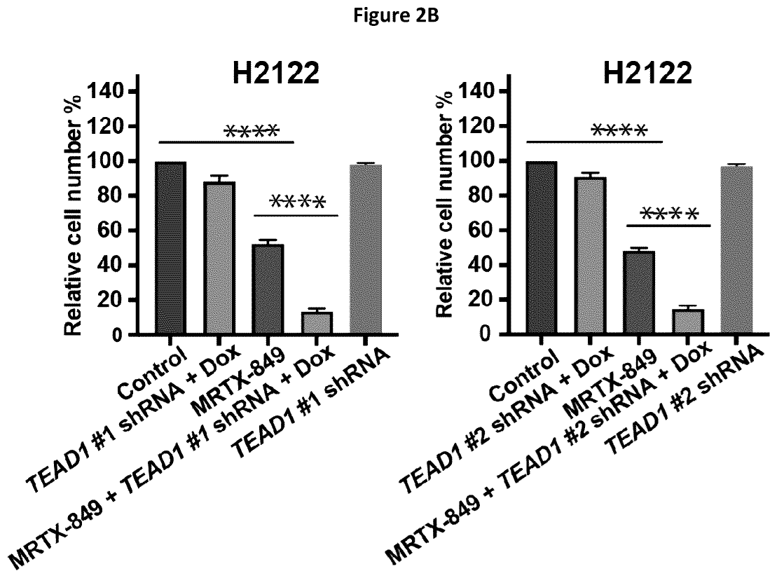

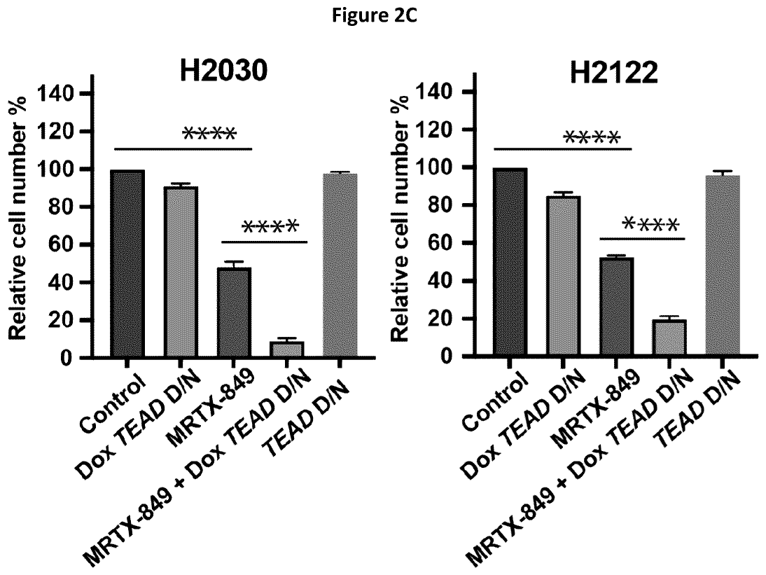

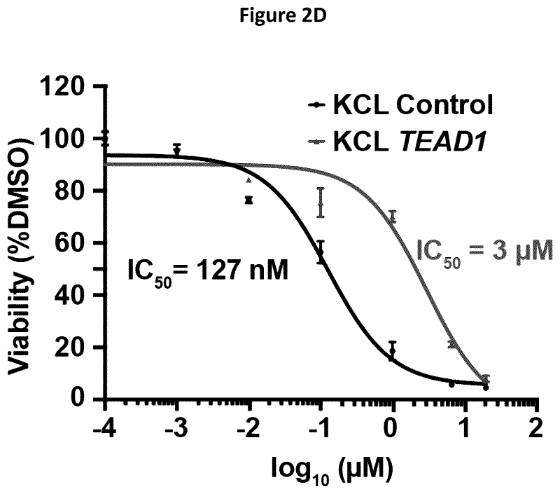

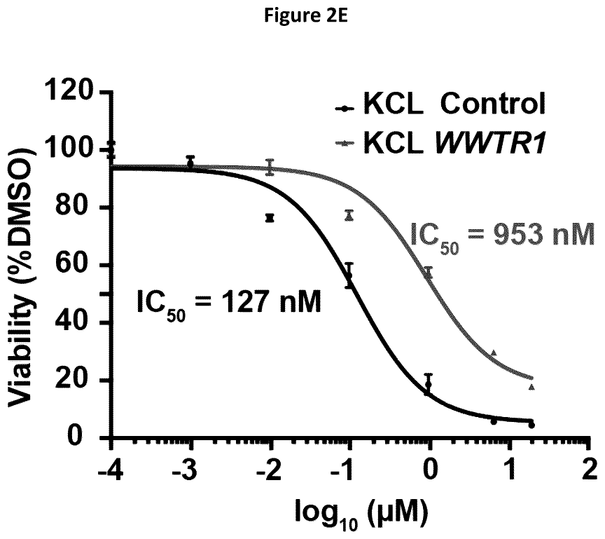

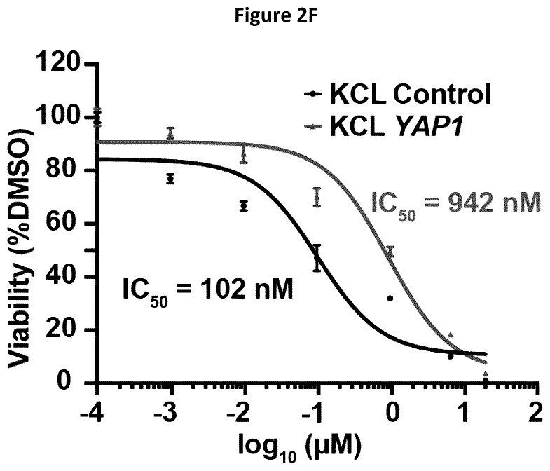

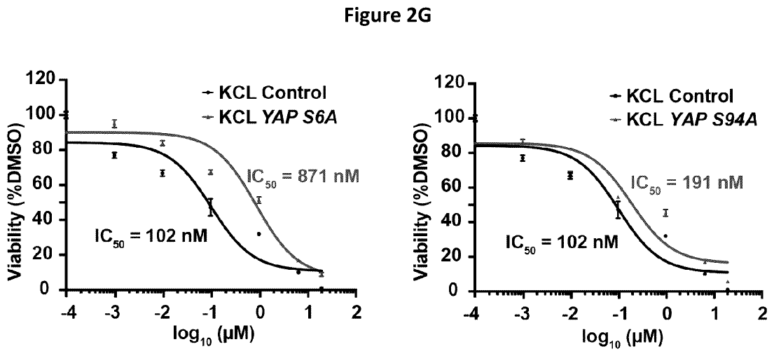

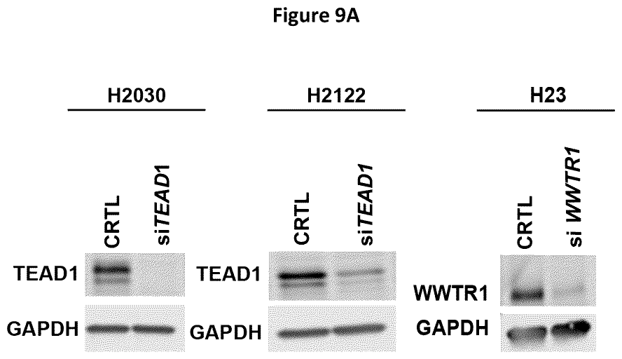

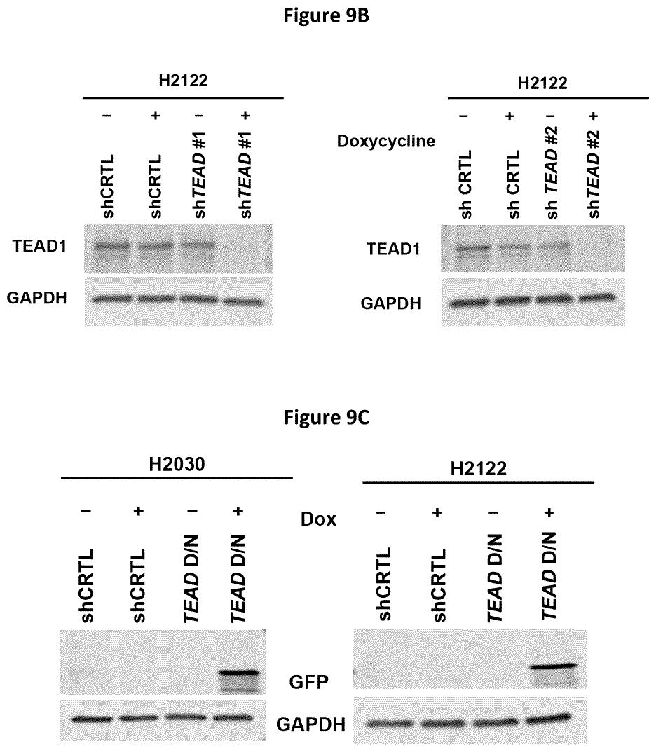

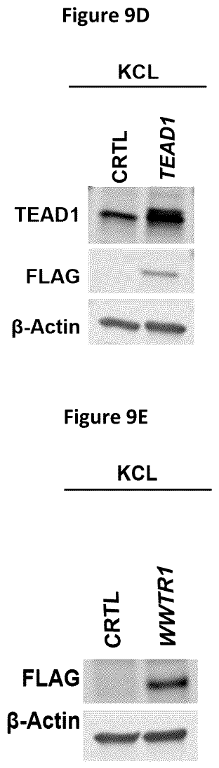

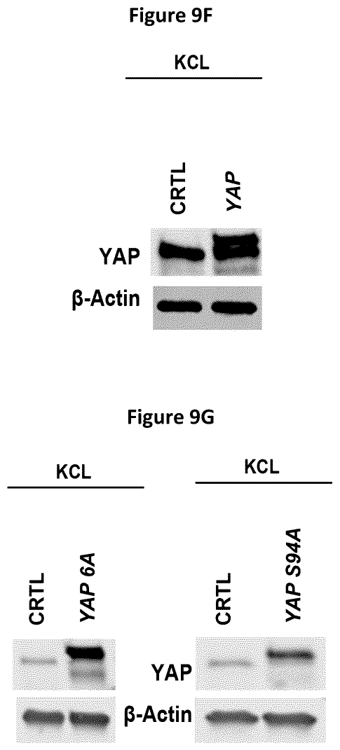

TEAD inhibitors are in clinical trials for NF2-mutant mesothelioma and other indications (e.g., NCT05228015 and NCT04665206) (24). YAP/TAZ pathway activation is also a known mechanism of resistance to other targeted therapies, including RAF^{V600E} and IVIK inhibitors (25). Comporting with the screen results, TEAD1 or WWTR1 depletion by siRNAs (“Smartpool”) or doxycycline-inducible shRNAs enhanced MRTX-849 efficacy in multiple lines (FIGS. 2A and 2B; FIG. 9A-B). Inducing expression of a dominant negative mutant of TEAD1 (26) had similar effects (FIG. 2C; FIG. 9C). Conversely, overexpression of TEAD1 (FIG. 2D; FIG. 9D), WWTR1 (FIG. 2E; FIG. 9E), YAP1 (FIG. 2F; FIG. 9F), or a constitutively active, nucleus-restricted form of YAP1 (27), YAP^{S6A}, (FIG. 2G; left panel; FIG. 9G) in “KCL” cells (derived from KRAS^{G12C};Stk11−/− mice; see Methods) caused drug resistance. By contrast, YAP^{S94A}, which encodes a mutant unable to associate with TEAD family members (28), did not affect MRTX-849 response (FIG. 2G, right panel, FIG. 9G). Collectively, these findings indicate that YAP1 and/or TAZ, acting in the nucleus by binding TEAD1 (or other TEAD family members), can antagonize G12Ci action and cause resistance.

### Example 3. RHO Directs ROCK-Dependent Nuclear Localization of YAP in Response to MRTX-849

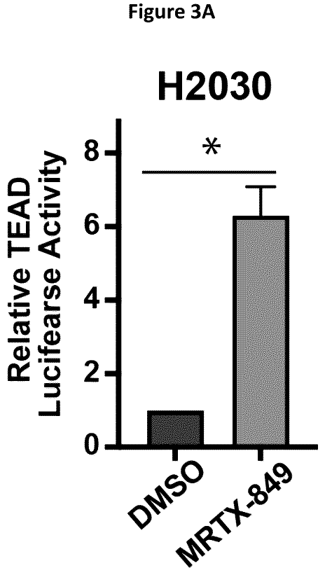

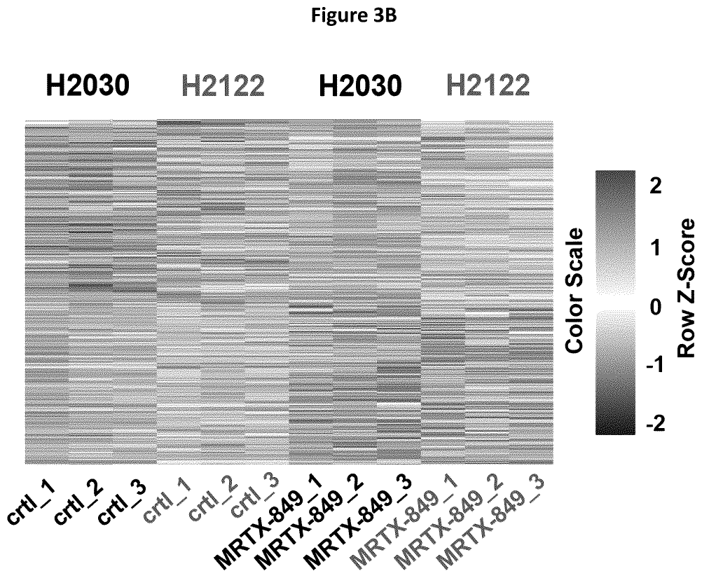

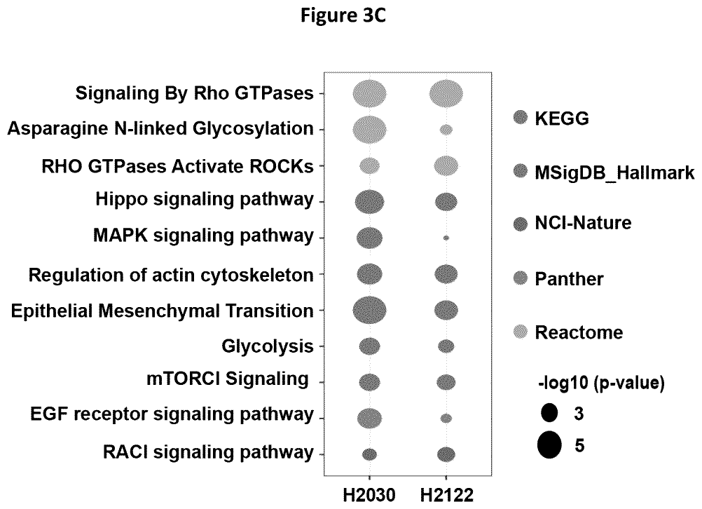

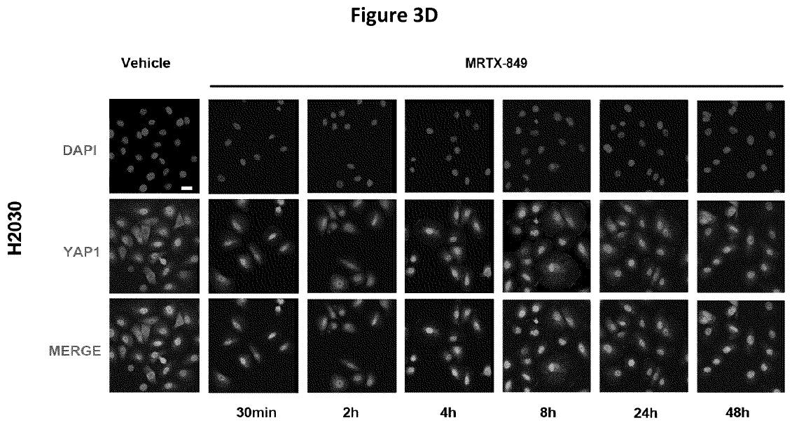

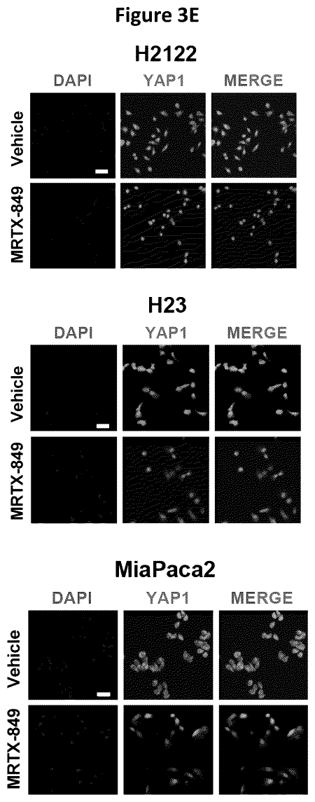

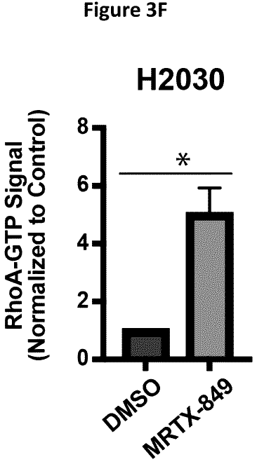

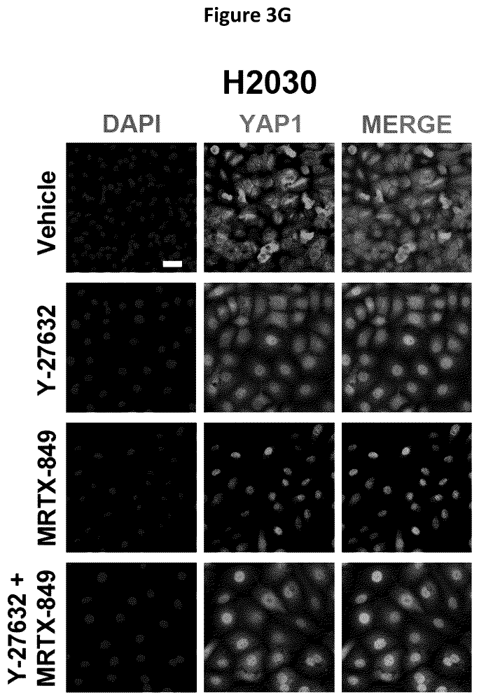

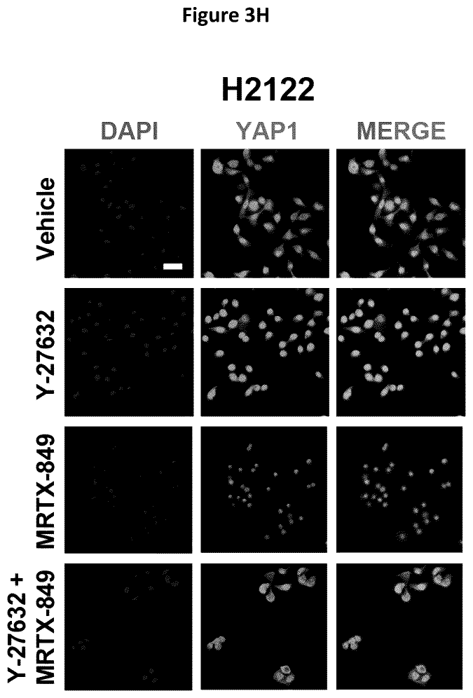

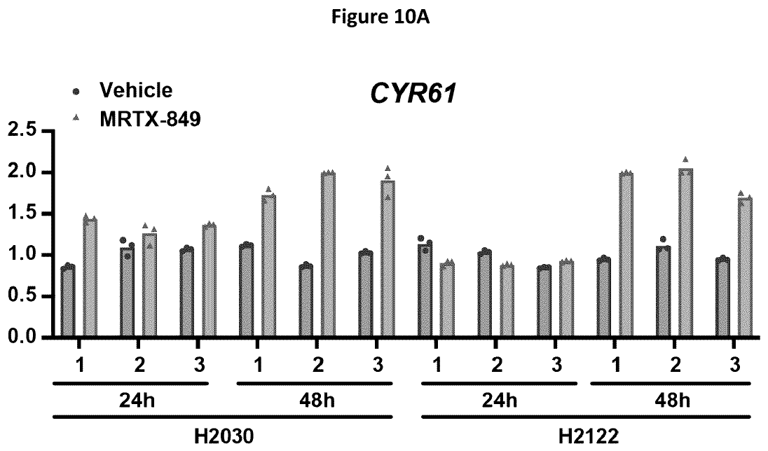

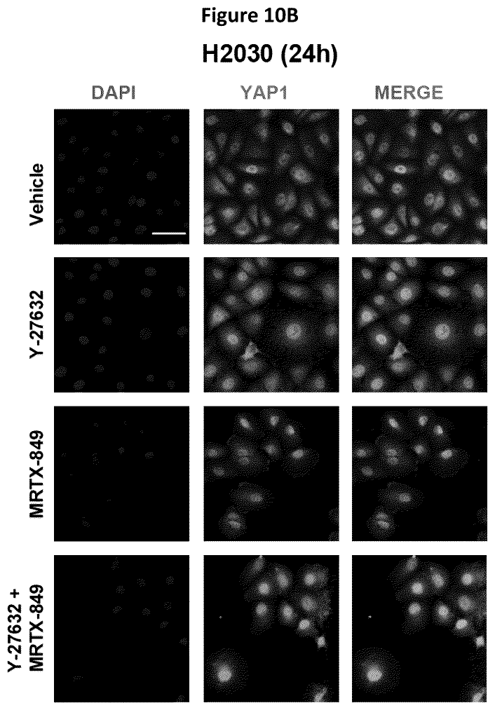

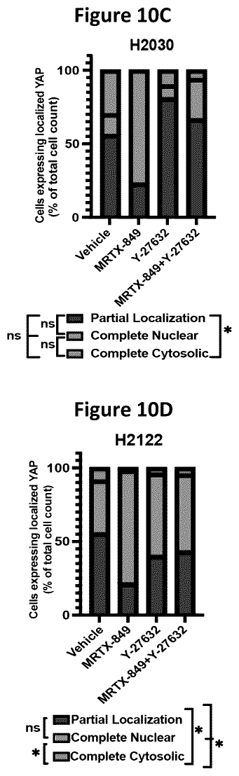

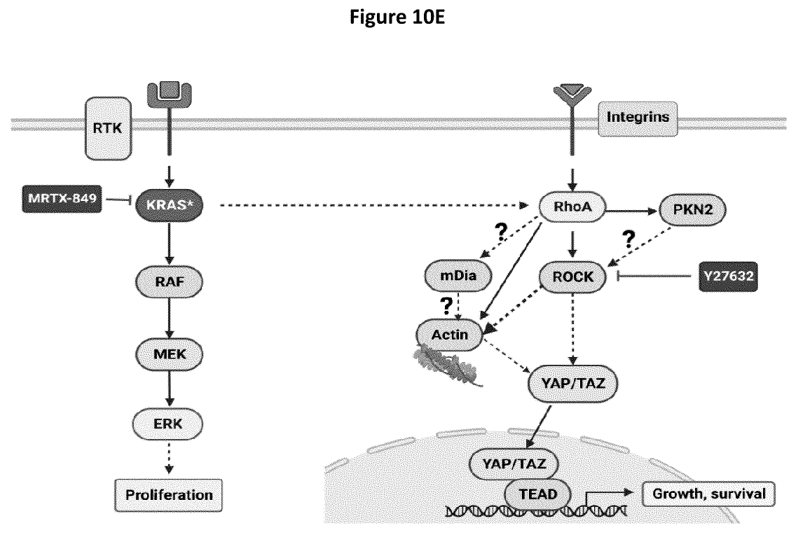

Next was studied whether YAP/TAZ/TEAD activity is modulated by G12Ci treatment. H2030 cells were transiently transfected with 8×GTIIC-Luci, a luciferase reporter driven by TEAD binding sites, and exposed to MRTX-849 (IC₅₀ dosage for 48 hours) or left untreated. Notably, TEAD reporter activity increased by >6-fold in G12Ci-treated cells (FIG. 3A). Moreover, transcript levels of the YAP/TEAD-inducible gene CYR61 increased after 48h of MRTX-849 treatment in two NSCLC lines tested (FIG. 10A). To assess the global effects of G12Ci treatment on the NSCLC transcriptome, RNAseq was performed on two MRTX-849-treated lines (H2030 and 2122). Unsupervised clustering clearly separated control and treated groups in both lines (FIG. 3B, Table 3). Remarkably, many upregulated pathways, including Hippo signaling, conformed to the SL pathways identified by the screen described herein (compare FIGS. 1E and 3C).

YAP/TAZ activity is controlled by multiple mechanisms, including the MST (and MAP4K)/LATS kinase cascade, RHO/ROCK signaling, and possibly FAK/SRC-catalyzed tyrosine phosphorylation of YAP (29,30). Most of these pathways regulate nuclear translocation of YAP/TAZ. MRTX-849 treatment also increased levels of nuclear YAP in H2030 cells, beginning at 4 hours and peaking at 24-48 hours (FIG. 3D, FIG. 10B). Similar results were obtained with other NSCLC lines (H2122, H23) and with the PDAC cell line MiaPaca2 (FIG. 3E).

The delayed kinetics of YAP nuclear translocation following MRTX-849 treatment suggested a transcription-dependent process. As noted above, RHO/ROCK signaling can promote YAP activation; also, Rhoa is required for mutant KRAS-induced NSCLC in mice (31). Moreover, genes annotated as “Signaling by RHO GTPases”, including several RHO family members (RHOA, RHOB, RHOD), RHO-guanine nucleotide exchange factors (RHO-GEFs: VAV2, TRIO, PICALM, ARGGEFIOL, AKAP13), and possible RHO-GEFs (ARHGEF40, PLEKHG4) were induced in both cell lines (FIG. 3C, Table 3). RHO-mediated YAP activation is thought to be mediated via integrin activation, cytoskeletal reorganization, and actomyosin contractility (32,33). FEMRT2, encoding KINDLIN-2, which mediates integrin activation, was also induced in both lines, as was LIMK2, which regulates COFILIN and promotes F-actin formation, multiple myosin genes (e.g., MYL6, MYH9, MYH10, MYH14, MYL9, MYL12B, MYO6, and others), and MYLK, which encodes myosin light chain kinase, a ROCK target that regulates myosin contractility (Table 3).

Collectively, these findings suggested that MRTX-849 treatment evokes a transcriptional program leading to increased integrin activation, cytoskeletal reorganization, actomyosin contractility, RHO activation and, consequently YAP/TAZ pathway activation (FIG. 10C). Consistent with this notion, RHO activity (assessed by G-LISA) increased following MRTX-849 treatment (FIG. 3F); genes annotated as “Signaling by RHO GTPases” were enriched in the SL screens (FIG. 1E), and the ROCK inhibitor Y27632 inhibited YAP nuclear translocation induced by MRTX treatment for 24 hr (FIG. 10B) and to an even greater extent after 48h of treatment (FIGS. 3G-H). Y27632 addition also increased MRTX-849 efficacy in two cell lines tested (FIG. 3I, FIG. 10C).

### Example 4. Mechanisms of Resistance to G12C/SHP2 Co-Inhibition in RAS-Driven NSCLC

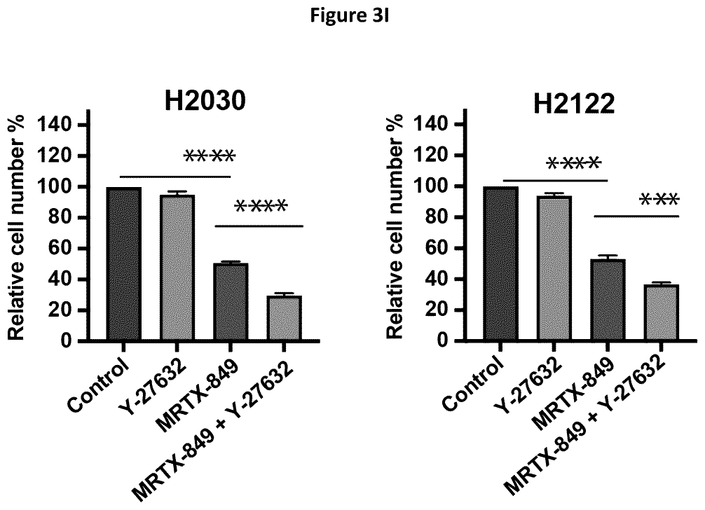

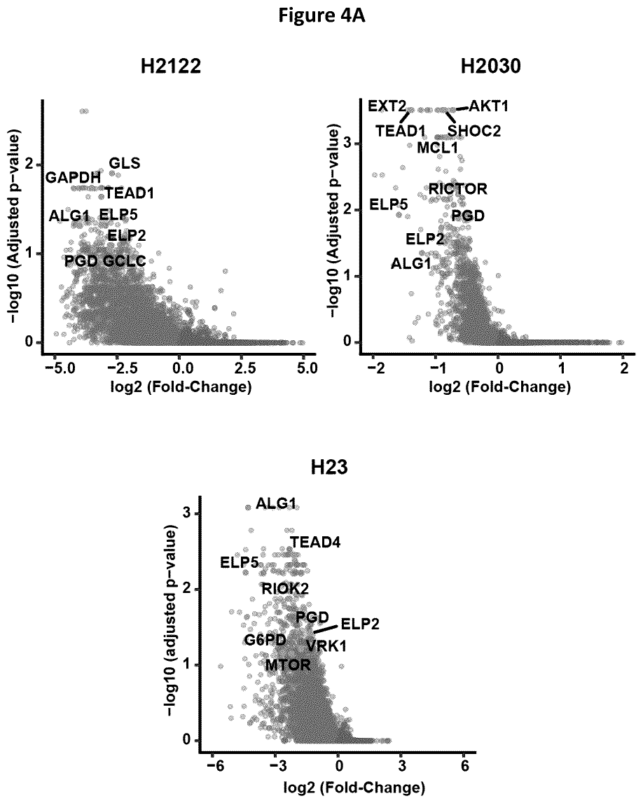

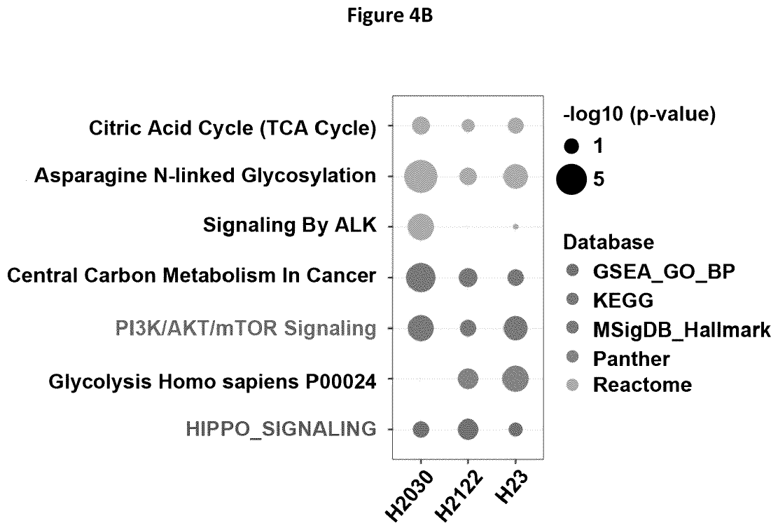

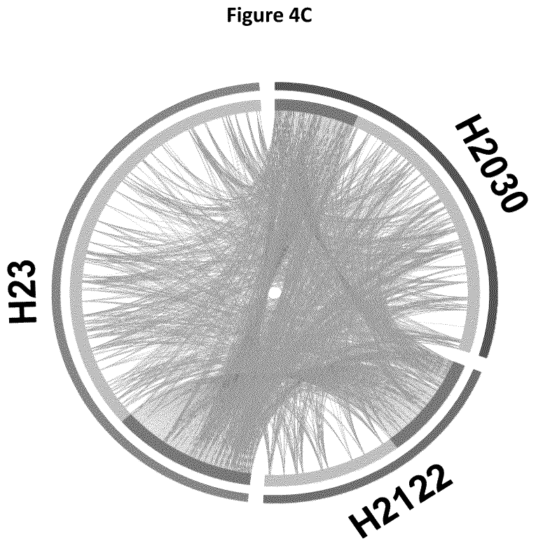

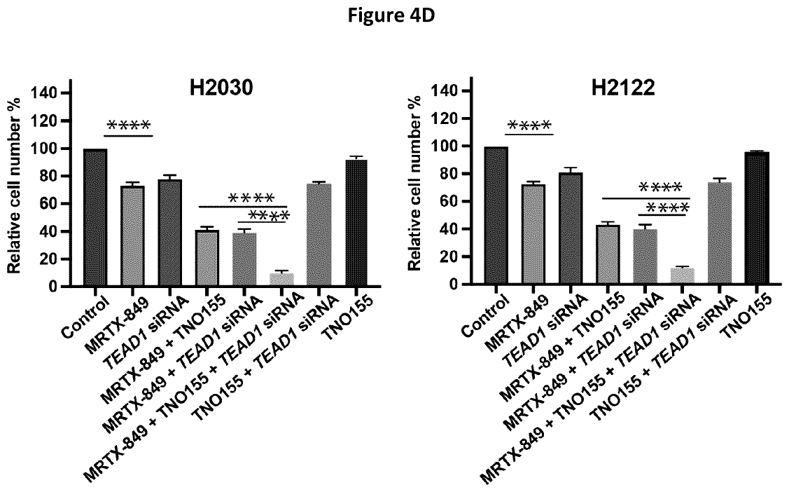

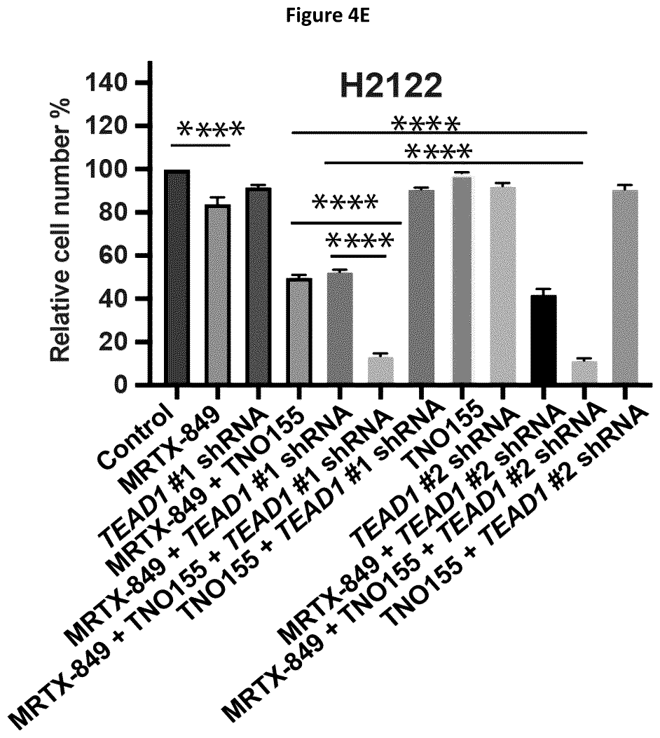

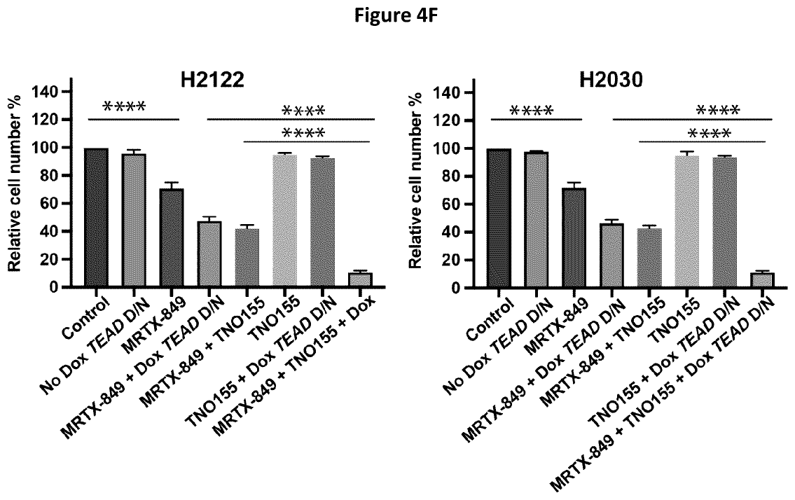

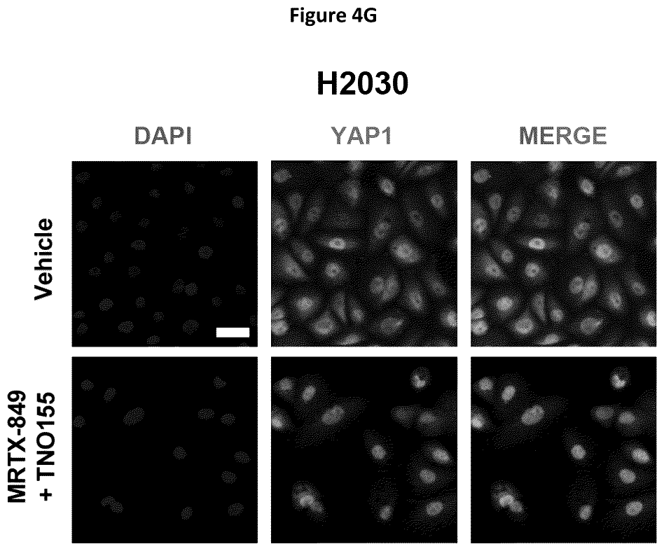

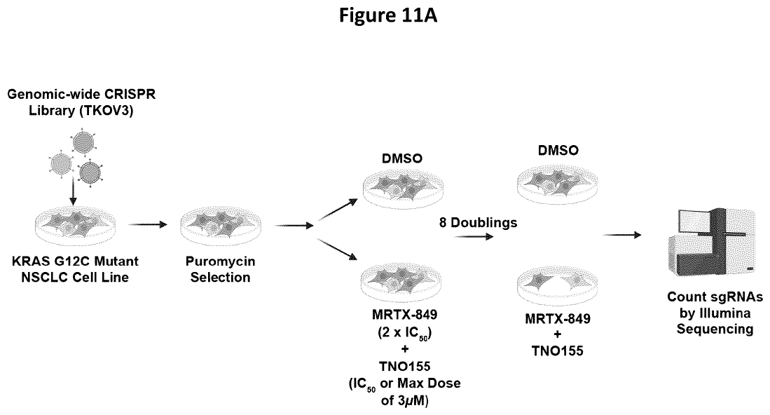

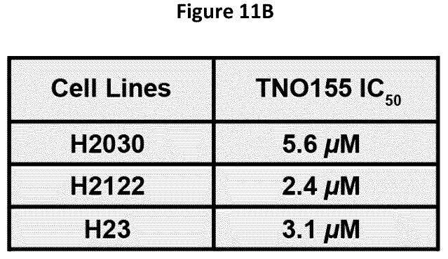

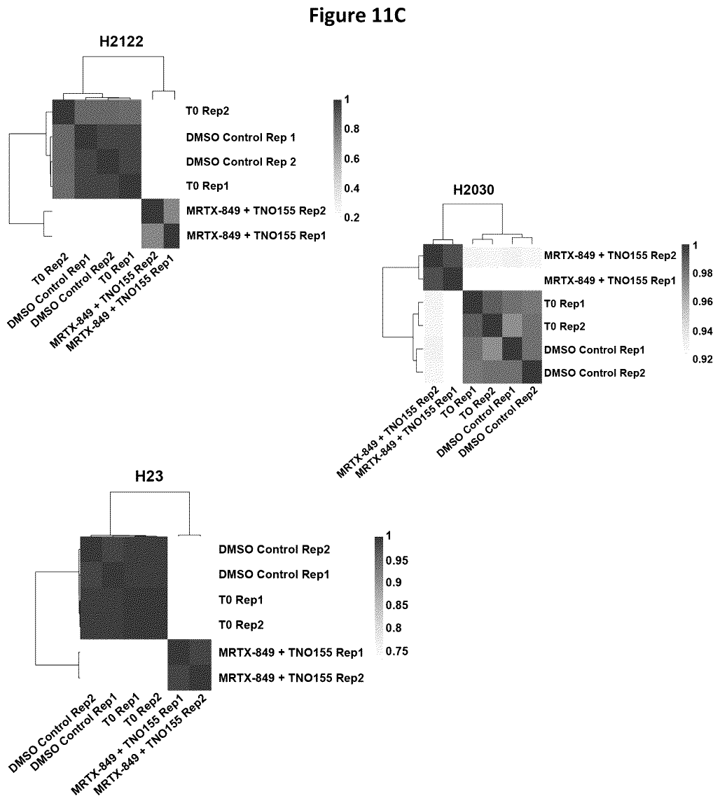

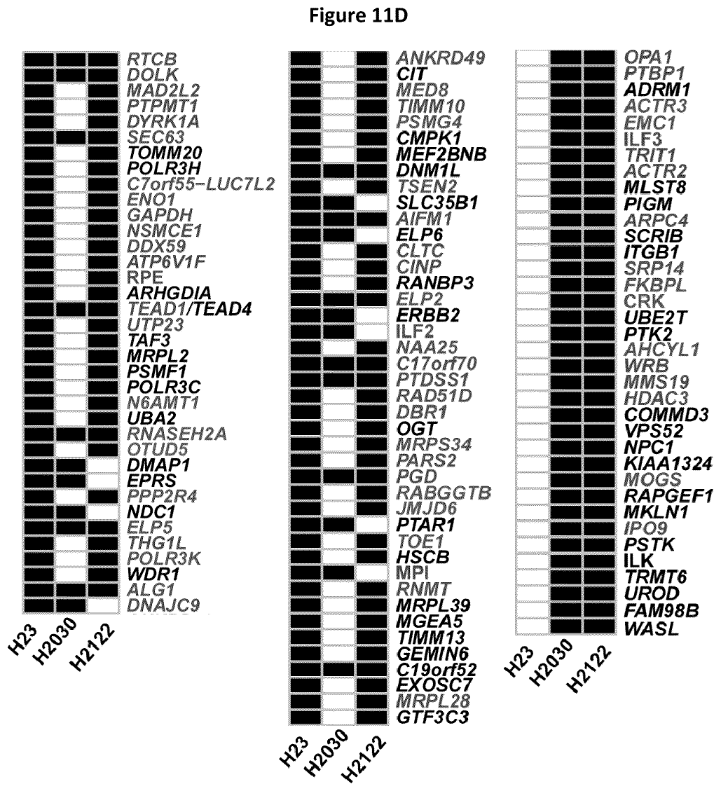

It had been reported previously that SHP2 inhibition enhances G12Ci action in vitro and in mice (34-36), and multiple SHP2i are in clinical trials for KRAS^{G12C}-mutant tumors and other indications (NCT05480865, NCT03565003, NCT04916236, NCT04699188) (37). To test whether G12Ci/SHP2 combinations might show efficacy in patients, CRISPR/Cas9 screens were performed on combination-treated H2122, H2030, and H23 cells; HCC44 cells were too sensitive to the combination to obtain meaningful results. For these experiments, MRTX-849 was added at 2×IC₅₀, and the clinical grade SHP2i TNO-155 was administered at its IC₅₀ or 3 uM, its maximal dose (FIG. 11A-B). For comparison, SL screens were also carried out on the same lines treated with TNO-155 (Table 4). Again, screen quality was high (FIG. 11C), and multiple shared and unique “hits” were identified (FIG. 4A-C). Several genes were SL with MRTX-849 alone and with MTRX-849/TNO-155 (indicated by red color); others, though, were unique to one treatment (FIG. 11D). Similarly, shared SL pathways (PI3K/mTOR signaling, Glycolysis, N-linked glycosylation) were identified, but those (Signaling by ALK, Central Carbon Metabolism) unique to combination-treated cells also emerged (FIG. 4B and FIG. 11D). Notably, TEAD1 or TEAD4 were hits in the MIRTX849/TNO155 screen (FIG. 11D), and genes annotated “Hippo signaling” were enriched as SL in the combination screen (FIG. 4B).

TEAD1 and TEAD4 were validated as SL with MRTX-849/TNO155 using si- and shRNAs and dominant negative TEAD via studies analogous to those used for MRTX-849 alone (FIG. 4D-F). It was also observed that combination therapy induced YAP nuclear translocation, consistent with a shared mechanism (FIG. 4G). These results suggest that TEAD inhibition could augment this drug combination as well as single agent MRTX-849.

TABLE 3
RNA-seq analysis of H2030 and H2122 cells treated with MRTX-849 (adagrasib) or vehicle for 48 hr.

| | | | | | | |
|---|---|---|---|---|---|---|
| row | ctrl | MRTX | median1 | median2 | sd1 | sd2 |

Combined differentially expressed genes (DEG) for H2122 and H2030; data used to generate the heatmap for FIG. 3B.

| | | | | | | |
|---|---|---|---|---|---|---|
| SAT1 | 2793.69082 | 1139.60273 | 2820.79082 | 1132.46373 | 83.2679731 | 60.2735324 |
| ITGA2 | 5498.00763 | 1985.54463 | 5317.14951 | 1983.31229 | 386.548578 | 56.8414795 |
| NT5E | 12156.6565 | 2044.77546 | 12006.3596 | 2030.13053 | 2159.34017 | 210.833486 |
| PSME1 | 3814.76961 | 5480.9375 | 3800.26157 | 5495.08921 | 72.7922869 | 172.035462 |
| GRAMD1B | 26131.0399 | 9388.18998 | 26439.5148 | 9429.07591 | 1149.6091 | 1206.78396 |
| TOR4A | 2316.7774 | 1387.33532 | 2301.72482 | 1380.80852 | 86.6696305 | 82.3113931 |
| JDP2 | 276.459321 | 575.605406 | 273.528892 | 586.516425 | 19.7224526 | 31.51313 |
| DUSP8 | 184.26086 | 418.605796 | 184.435971 | 419.232943 | 6.24341701 | 35.9589204 |
| UBALD2 | 673.233509 | 281.140543 | 696.171257 | 277.018293 | 58.9646675 | 24.0943538 |
| ETV4 | 1289.50394 | 70.6362343 | 1260.37205 | 67.3283676 | 352.90813 | 30.1537302 |
| SPTAN1 | 13624.8957 | 17827.2035 | 13482.1056 | 17891.4205 | 489.398276 | 413.520179 |
| TPRA1 | 1298.53795 | 2151.38691 | 1314.44125 | 2127.62277 | 84.8094705 | 111.974053 |
| UPP1 | 2488.63031 | 1209.67278 | 2501.84674 | 1203.78951 | 276.811045 | 57.2176884 |
| ARPC5 | 5555.72118 | 7309.03371 | 5518.08394 | 7289.83804 | 195.102988 | 195.776358 |
| HLA-C | 7652.74117 | 12155.1826 | 7695.81994 | 12070.4929 | 482.806105 | 579.6877 |
| DAPK1 | 1312.54349 | 2729.03593 | 1302.25883 | 2756.10662 | 135.876572 | 216.722496 |
| AC004585.1 | 106.723773 | 15.5683804 | 106.79314 | 15.0858933 | 13.2372861 | 6.38799774 |
| VPS39 | 2613.38048 | 3447.09372 | 2595.25183 | 3456.59928 | 82.8427988 | 107.036364 |
| ERV3-1 | 198.373405 | 403.482028 | 197.414237 | 415.310718 | 12.6398107 | 36.7323324 |
| UBLCP1 | 2404.81829 | 1813.5196 | 2412.97729 | 1839.27531 | 34.4912922 | 85.5697762 |
| ARHGEF17 | 1386.55582 | 2263.65163 | 1371.23657 | 2297.65255 | 46.5707889 | 196.590752 |
| SMAP2 | 1385.48293 | 1018.777 | 1362.80955 | 1012.64696 | 53.2299958 | 31.4319058 |
| FLNB | 30742.1934 | 25080.6983 | 30790.6227 | 25046.81 | 724.958609 | 745.545117 |
| IL4R | 1407.72053 | 779.200314 | 1409.66752 | 776.846393 | 157.890882 | 21.9492266 |
| KCTD15 | 1046.15447 | 1674.65232 | 1022.69496 | 1699.17454 | 75.450743 | 103.855468 |
| SPRY2 | 760.249479 | 209.495461 | 785.220356 | 212.481776 | 166.314261 | 34.3190446 |
| SPRED1 | 1693.84858 | 652.084391 | 1748.69872 | 689.134691 | 164.929825 | 118.745473 |
| TMEM54 | 960.009651 | 1252.79827 | 957.280876 | 1264.15376 | 33.9795607 | 29.8162597 |
| GLB1 | 1749.81555 | 2553.93669 | 1755.78054 | 2578.25189 | 98.8799751 | 129.564394 |
| DUSP5 | 476.012038 | 248.935773 | 482.086058 | 245.869301 | 26.6661191 | 31.4784213 |
| SLC6A9 | 544.598281 | 911.920279 | 557.723112 | 887.671663 | 37.58291 | 74.544187 |
| MANBA | 718.650016 | 1049.3566 | 720.235551 | 1039.61834 | 24.4334943 | 68.2710477 |
| CELSR1 | 3237.26041 | 4665.43337 | 3193.43123 | 4629.83894 | 141.029921 | 295.766533 |
| PTPN12 | 7287.28748 | 4506.02313 | 7326.31913 | 4448.86603 | 305.511977 | 435.656055 |
| IRF1 | 442.525109 | 668.428698 | 450.523515 | 683.58352 | 24.0463991 | 34.5848934 |
| TXNRD1 | 61161.3046 | 73866.1477 | 61130.5208 | 73982.3144 | 2160.21342 | 1115.90093 |
| MAN1B1 | 2468.94467 | 3074.10583 | 2443.77187 | 3083.00655 | 89.2551162 | 71.0686139 |
| HMMR | 3168.51257 | 2286.65393 | 3155.64352 | 2283.13707 | 68.4907882 | 150.490467 |
| PTPN3 | 2090.30676 | 1627.39778 | 2095.95052 | 1620.93471 | 52.0348796 | 67.5430013 |
| TCTN1 | 571.878076 | 767.30775 | 575.220396 | 775.438033 | 14.2442777 | 21.5060792 |
| HIF1A | 10357.915 | 7629.72585 | 10325.7752 | 7709.38313 | 252.400272 | 468.349769 |
| APOL2 | 1107.69232 | 1676.01483 | 1101.12602 | 1678.30979 | 94.0943197 | 56.3843153 |
| PACS2 | 2224.25789 | 3070.58816 | 2254.42891 | 3103.33456 | 70.968727 | 188.895819 |
| MYORG | 1602.45029 | 2475.21852 | 1567.09213 | 2452.6617 | 111.593963 | 176.038882 |
| SRPK1 | 3996.11203 | 3030.22081 | 4060.00361 | 3005.06882 | 126.724913 | 157.625269 |
| AC090409.1 | 47.3363334 | 9.67257961 | 47.0015927 | 8.99156243 | 3.48754775 | 3.16668581 |
| DUSP7 | 1085.04155 | 724.750764 | 1066.55504 | 734.238228 | 56.212216 | 55.0877784 |
| PLEKHA8 | 810.024878 | 611.180403 | 811.316972 | 615.57102 | 24.7371917 | 19.5032137 |
| KBTBD2 | 1994.18452 | 1420.38755 | 1984.30898 | 1388.94892 | 44.6343743 | 108.01265 |
| APMAP | 3461.24263 | 4115.58847 | 3465.69408 | 4136.71554 | 75.6949881 | 113.236764 |
| TAGLN2 | 42433.9439 | 51897.7132 | 42195.2668 | 52376.6133 | 1058.91366 | 2000.55344 |
| LRCH1 | 752.903934 | 551.791904 | 733.529924 | 548.434683 | 36.9271646 | 13.3557908 |
| ELMO3 | 1616.92455 | 2092.44313 | 1628.12717 | 2097.82384 | 72.7914752 | 66.1919267 |
| HIBADH | 2886.83988 | 3464.49497 | 2873.13953 | 3477.19106 | 85.8123768 | 91.7967515 |
| PHKB | 4442.55598 | 5160.37954 | 4438.31573 | 5163.16529 | 107.012808 | 96.6439644 |
| TCF7L2 | 1582.11425 | 1235.37889 | 1569.41123 | 1230.57729 | 52.6942556 | 49.5054216 |
| LUCAT1 | 1870.02385 | 970.320734 | 1843.84373 | 960.074685 | 105.523894 | 157.333823 |
| RHOA | 18337.4368 | 23030.1177 | 18368.0686 | 23060.8535 | 476.082987 | 1147.89973 |
| MLX | 3513.29106 | 2073.92263 | 3517.26934 | 2066.66118 | 332.765277 | 211.83344 |
| PPP1R13L | 1710.14028 | 2938.34898 | 1728.30292 | 2940.44519 | 236.944276 | 115.196319 |
| RGS2 | 1819.07226 | 533.27569 | 1843.87637 | 516.405286 | 473.638684 | 120.263854 |
| DAAM1 | 1011.41856 | 1297.6145 | 1001.50148 | 1304.27139 | 46.0941627 | 45.8324928 |
| MYL6 | 11952.3664 | 17128.2653 | 11978.5043 | 17546.3947 | 135.937088 | 1577.0687 |
| CRYAB | 4.0870167 | 45.7150871 | 3.59133409 | 41.7356929 | 2.11218183 | 16.3015337 |
| DPY19L1 | 4795.61124 | 3703.43505 | 4798.13048 | 3725.71505 | 128.645702 | 220.735971 |
| UBE2Q2 | 2354.58776 | 2880.41982 | 2332.78312 | 2900.89491 | 50.5190537 | 122.028288 |
| DDR1 | 4166.17072 | 5491.20991 | 4170.01404 | 5529.23975 | 57.5791515 | 381.379002 |
| MXD4 | 644.554615 | 1076.79827 | 647.952003 | 1056.3889 | 43.353718 | 131.026397 |
| APH1A | 4460.54181 | 5343.82576 | 4477.33902 | 5346.01129 | 110.111051 | 197.481871 |
| TRAM1 | 5375.00193 | 9879.76651 | 5344.42029 | 9730.03477 | 664.471437 | 1248.57275 |
| NIPSNAP3A | 397.090399 | 587.118614 | 402.79951 | 579.084232 | 29.3105598 | 37.3603777 |
| KIF14 | 2212.586 | 1694.60627 | 2235.24609 | 1670.96278 | 83.8952037 | 98.142583 |
| SCAT8 | 70.3115646 | 26.3022368 | 74.0758714 | 28.2410048 | 8.22314673 | 5.03508891 |
| MRGBP | 2294.99349 | 1658.63403 | 2275.77732 | 1651.22886 | 138.213645 | 106.265794 |
| SPRY4-AS1 | 53.3098319 | 13.2480713 | 52.9220496 | 13.3462525 | 7.69431365 | 5.34621979 |
| RCN1 | 1531.10771 | 1871.03586 | 1512.37018 | 1862.66254 | 63.9010595 | 50.0744957 |
| TMEM14A | 1117.15119 | 1326.76027 | 1121.49559 | 1333.97623 | 21.3964346 | 23.0627016 |
| GPX1 | 5586.25059 | 4152.75872 | 5559.87043 | 4096.83178 | 250.226809 | 295.810063 |
| BCAR3 | 3015.87627 | 2133.1172 | 2968.29385 | 2130.83918 | 215.716019 | 138.993396 |
| STX7 | 1009.18479 | 1274.9636 | 1009.44326 | 1288.00863 | 38.8787012 | 52.1774375 |
| LRSAM1 | 850.25641 | 1068.11529 | 851.08187 | 1067.74168 | 25.4509537 | 45.1757792 |
| EPHA2 | 5386.17652 | 2908.85198 | 5349.15123 | 2932.3934 | 959.008145 | 126.196846 |
| CASC8 | 91.9970711 | 31.379219 | 93.4875246 | 33.448678 | 12.2562154 | 8.63915348 |
| SERPIND1 | 38.9342587 | 2.5479069 | 38.246552 | 2.42277222 | 12.0173108 | 2.54097461 |
| CTSL | 17820.5079 | 8080.71084 | 17929.1027 | 8019.76171 | 734.955372 | 1939.01737 |
| STAMBPL1 | 717.058363 | 294.926323 | 727.296878 | 298.925776 | 171.083542 | 31.2737548 |
| ANKRD2 | 171.434482 | 378.396473 | 174.536687 | 386.199216 | 8.48279823 | 81.361864 |
| POLE2 | 822.322215 | 597.147147 | 824.347861 | 593.814369 | 27.9156004 | 50.4648512 |
| UTP11 | 2063.7033 | 1643.12756 | 2079.42441 | 1629.84721 | 70.5110543 | 86.1470458 |
| SMAD6 | 1033.73511 | 1468.49835 | 1034.62593 | 1480.52562 | 54.8733336 | 129.148819 |
| TINAG | 56.2612977 | 9.75851394 | 55.2377819 | 10.1011236 | 18.909428 | 3.22681314 |
| AC012313.1 | 316.09425 | 212.190332 | 318.275856 | 211.824362 | 21.3189354 | 14.7554004 |
| PIGT | 5262.44961 | 6536.73667 | 5250.3839 | 6642.52304 | 117.611898 | 379.505511 |
| ADSSL1 | 168.896595 | 280.942116 | 166.946981 | 271.016448 | 17.167168 | 27.7104552 |
| PPIC | 670.683448 | 892.611666 | 669.812124 | 875.493122 | 40.5699569 | 47.3506828 |
| DNAJB11 | 1439.15592 | 1192.26762 | 1419.13426 | 1204.70426 | 53.516412 | 34.0140713 |
| ERBB2 | 3874.79154 | 6221.28804 | 3906.0585 | 6184.01246 | 398.941484 | 667.974444 |
| PTTG1 | 3809.09613 | 2985.60317 | 3809.67156 | 2971.10849 | 166.564731 | 169.003462 |
| RIPK4 | 1574.66965 | 926.676915 | 1598.23471 | 918.620762 | 252.879596 | 29.5177643 |
| SLCO2B1 | 10.1039685 | 42.0106506 | 11.484783 | 40.593534 | 3.95367104 | 9.1349176 |
| HSD17B4 | 3302.81011 | 3874.60424 | 3296.58208 | 3843.6155 | 87.3603977 | 140.252206 |
| GRHPR | 2291.12112 | 2748.84176 | 2298.36038 | 2739.69625 | 109.457398 | 54.1219949 |
| DGCR2 | 2356.45471 | 3289.3072 | 2360.07875 | 3249.62674 | 131.359791 | 291.532877 |
| HMGA1 | 22413.1748 | 13357.7115 | 22279.8043 | 13294.1953 | 3513.11416 | 914.564369 |
| FARP2 | 1125.70732 | 1403.31544 | 1119.0868 | 1411.88385 | 54.1677009 | 59.3404042 |
| MRPS18B | 2992.6828 | 2509.83129 | 3017.51584 | 2519.15145 | 93.5287956 | 101.601826 |
| AC102953.2 | 378.735974 | 550.44059 | 376.652602 | 552.136741 | 33.716682 | 39.0961841 |
| SLC16A14 | 2907.64754 | 1841.45888 | 2850.0256 | 1815.1707 | 222.365797 | 238.691565 |
| UPK3B | 39.4161973 | 328.325414 | 38.0969928 | 317.67392 | 10.2541415 | 207.713477 |
| SAMD11 | 124.402853 | 409.707699 | 117.503968 | 416.038201 | 27.8451685 | 135.760952 |
| P4HTM | 1219.85198 | 1648.04998 | 1233.29227 | 1635.36218 | 94.738611 | 85.1283393 |
| OAF | 646.835396 | 466.831901 | 642.388196 | 469.455031 | 32.4890947 | 40.6972495 |
| RHOB | 2951.74754 | 5654.85641 | 2939.66935 | 5637.22499 | 157.707854 | 1218.86288 |
| TINCR | 117.081673 | 212.044913 | 112.680931 | 209.693613 | 13.4128055 | 29.7584032 |
| ABALON | 100.911347 | 55.6179877 | 98.989033 | 55.2440033 | 9.13514358 | 3.39533603 |
| RBMS2 | 4555.92679 | 3562.85836 | 4582.34168 | 3527.45322 | 133.388755 | 260.910458 |
| SPATS2L | 2261.9178 | 2887.31858 | 2267.90042 | 2864.50469 | 152.201431 | 111.680225 |
| AMBRA1 | 795.711882 | 970.969069 | 800.915345 | 970.623931 | 22.376296 | 43.2265752 |
| RPRD1B | 1477.36961 | 1245.08309 | 1475.02479 | 1241.0797 | 45.4508317 | 39.0137951 |
| FOSL2 | 7256.42889 | 10766.9654 | 7283.45317 | 10756.7103 | 938.415287 | 383.603859 |
| RRM2 | 19524.6242 | 16110.2772 | 19282.7068 | 15840.2864 | 930.446911 | 650.97233 |
| FOSL1 | 2134.60264 | 826.583363 | 2113.09222 | 799.60235 | 633.111881 | 135.369642 |
| CDK17 | 992.67635 | 771.862485 | 986.71608 | 772.086338 | 48.14527 | 45.8889161 |
| ACKR3 | 5.29031486 | 29.0334623 | 6.05819369 | 27.3660599 | 3.06872149 | 7.13550352 |
| ATP6AP1L | 77.2360919 | 37.8003382 | 78.3759622 | 36.1348875 | 7.3800985 | 6.73013124 |
| TTC14 | 348.686369 | 524.484504 | 340.057018 | 530.610311 | 28.7110993 | 53.6921613 |
| TCTN3 | 2686.10026 | 3507.57084 | 2689.94635 | 3483.78295 | 187.986362 | 191.482873 |
| RGP1 | 2264.96985 | 1518.99266 | 2269.23634 | 1519.69095 | 279.996203 | 86.82462 |
| RPF1 | 1487.62705 | 1223.34354 | 1495.66606 | 1214.12556 | 63.2296773 | 47.9613125 |
| MR1 | 548.946713 | 820.20787 | 551.748396 | 815.270396 | 62.3364157 | 61.102338 |
| THUMPD3 | 1641.05308 | 1368.46086 | 1624.19902 | 1379.46342 | 73.9708966 | 37.0932919 |
| C5orf51 | 2693.54177 | 2184.25974 | 2685.94529 | 2204.26904 | 110.419999 | 119.944908 |
| CHMP7 | 2250.23612 | 1946.77149 | 2263.17176 | 1938.67518 | 69.6367203 | 54.7213344 |
| NHS | 893.513808 | 630.713681 | 909.662646 | 616.545479 | 51.6433788 | 65.6480697 |
| PDXK | 6700.80846 | 13593.4218 | 6657.91351 | 13539.6274 | 615.937755 | 3311.60097 |
| FKBP9 | 5581.7177 | 7183.71369 | 5586.82588 | 7027.97509 | 113.40973 | 600.57017 |
| EBNA1BP2 | 5154.95031 | 4327.97451 | 5128.27038 | 4295.79249 | 153.346749 | 212.882907 |
| GAS2L3 | 945.874902 | 741.889873 | 956.095472 | 760.677616 | 44.9811512 | 45.7906982 |
| PRR11 | 3143.82586 | 2585.07272 | 3148.14989 | 2604.23262 | 181.24306 | 66.400844 |
| EIF2B2 | 1387.64125 | 1061.78066 | 1375.1519 | 1062.12273 | 115.419777 | 31.228604 |
| SIL1 | 912.903037 | 1113.18249 | 893.868203 | 1125.73379 | 49.4947502 | 29.4476628 |
| CHD3 | 3835.42286 | 4897.38183 | 3834.8219 | 4875.19663 | 166.096399 | 351.121248 |
| RAB27A | 1712.8478 | 1375.93327 | 1682.93864 | 1364.40284 | 106.072695 | 50.3473747 |
| TMEM94 | 1484.56609 | 2043.87256 | 1476.50319 | 2027.72591 | 111.641962 | 173.962448 |
| PTPN13 | 582.588219 | 764.599729 | 590.216578 | 748.791863 | 43.494727 | 37.7988221 |
| PAIP2 | 1100.96681 | 853.205591 | 1094.24006 | 834.397938 | 50.8166781 | 62.1258305 |
| TRAPPC6A | 227.48809 | 429.98256 | 217.562947 | 426.357784 | 22.8425383 | 89.0636197 |
| CDK5R1 | 297.668711 | 187.383527 | 304.668855 | 180.14305 | 28.7019826 | 24.7592006 |
| SGPL1 | 4320.20092 | 5539.59538 | 4289.25148 | 5496.44394 | 333.901001 | 257.29636 |
| SUMF1 | 1307.3827 | 1884.81154 | 1302.63909 | 1887.71513 | 122.639864 | 182.036461 |
| CHP1 | 8977.25842 | 12935.5801 | 8925.86318 | 13046.8737 | 868.533179 | 1252.96507 |
| TM2D2 | 1005.788 | 1387.73022 | 995.384014 | 1382.78957 | 114.195785 | 48.283259 |
| TUFT1 | 2322.60239 | 4595.12543 | 2308.94908 | 4607.66981 | 171.627076 | 1169.20936 |
| EIF3G | 4035.67783 | 3644.24781 | 4012.94366 | 3651.17129 | 67.6738105 | 96.7224589 |
| CCT5 | 33583.5925 | 29634.9326 | 33859.5221 | 29627.0492 | 1133.55093 | 781.666355 |
| RNPEPL1 | 2063.9031 | 2570.55874 | 2049.91284 | 2552.4428 | 55.8102233 | 187.337009 |
| EVI2B | 11.4020331 | 0.14885256 | 10.7246053 | 0 | 2.27598767 | 0.36461283 |
| AREG | 3290.05272 | 1682.61 | 3257.3455 | 1671.72766 | 837.980885 | 93.6082236 |
| BMF | 127.048124 | 575.184978 | 129.079847 | 574.349724 | 17.7360981 | 319.508734 |
| COMMD6 | 599.142611 | 785.719674 | 598.636462 | 787.528188 | 23.9737456 | 65.4640496 |
| CUL3 | 3723.83633 | 3372.18247 | 3721.33519 | 3374.43757 | 43.0794006 | 87.7561394 |
| CXCL8 | 1110.57 | 196.013398 | 1143.78951 | 192.060338 | 550.215155 | 95.0059064 |
| GNG11 | 3129.45041 | 1698.14394 | 3139.62921 | .647.11879 | 704.120317 | 131.194934 |
| SOX12 | 1004.08544 | 1569.3834 | 1000.525 | 1594.92623 | 114.60343 | 196.538544 |
| ZNF275 | 598.024068 | 458.076185 | 591.752292 | 453.16306 | 31.7906915 | 32.462241 |
| SLC25A37 | 3502.87524 | 2835.95161 | 3435.47362 | 2848.54415 | 256.350079 | 54.0243239 |
| TOP1 | 6245.95742 | 4789.52888 | 6148.91194 | 4802.24606 | 558.002958 | 202.149249 |
| NAV3 | 1220.08331 | 671.511307 | 1211.16016 | 668.968542 | 210.406063 | 106.559007 |
| PLEKHM1 | 644.857843 | 778.301285 | 642.776283 | 781.061421 | 26.4538311 | 25.9631577 |
| MTCL1 | 1787.95598 | 2400.94396 | 1794.46823 | 2343.21597 | 64.1760015 | 251.908645 |
| MECR | 664.980582 | 804.415785 | 661.161675 | 811.535655 | 24.8455101 | 38.3033185 |
| WDR4 | 1148.95889 | 834.170785 | 1134.67949 | 827.085856 | 110.007608 | 59.3605711 |
| GPATCH11 | 711.180023 | 548.77441 | 706.700526 | 551.774597 | 36.8540735 | 40.1637727 |
| SH3GLB1 | 2643.71799 | 2925.53808 | 2650.75095 | 2894.80439 | 35.2072049 | 73.7030903 |
| MT-ND1 | 46097.5707 | 60756.0715 | 45918.6094 | 61807.5651 | 3829.31252 | 3982.20962 |
| MYH15 | 18.3846571 | 3.27757526 | 17.4352338 | 2.9143168 | 5.72595345 | 1.0783887 |
| PAPSS1 | 1182.92976 | 1436.66362 | 1187.67093 | 1457.10898 | 38.9479331 | 84.9084903 |
| RABL2B | 240.355746 | 320.023976 | 240.710954 | 313.711103 | 13.8764892 | 18.6408318 |
| ZER1 | 862.596994 | 1073.42751 | 858.178305 | 1060.91388 | 30.0847781 | 72.9445586 |
| PPFIBP2 | 112.057851 | 199.459521 | 114.817033 | 191.275824 | 21.3625452 | 20.8842812 |
| GDE1 | 2516.87906 | 2969.25272 | 2525.011 | 2967.70669 | 77.5100069 | 149.655141 |
| WBP2 | 3086.72461 | 2473.70006 | 3086.50091 | 2468.57543 | 219.019194 | 103.84808 |
| AL606500.1 | 54.7445587 | 16.5426583 | 54.9871086 | 14.5771073 | 16.4116672 | 5.49541928 |
| RHBDF2 | 707.115723 | 876.2056 | 702.638032 | 869.976846 | 39.7220548 | 44.2495547 |
| MMP24 | 42.5969247 | 334.03022 | 40.3857587 | 330.391321 | 17.3029726 | 235.141271 |
| KLF9 | 359.745994 | 452.301916 | 360.773368 | 448.529508 | 19.9398998 | 15.1811386 |
| DUSP6 | 514.137936 | 63.3701977 | 518.140672 | 62.8142546 | 100.344499 | 50.5052888 |
| GPD2 | 5083.61174 | 4206.91251 | 5066.0273 | 4200.73134 | 308.631263 | 154.658017 |
| SHB | 600.656804 | 425.139052 | 601.255338 | 432.256833 | 70.4190073 | 19.5754704 |
| TNNC1 | 10.0337652 | 89.9107663 | 10.7118012 | 88.7352267 | 1.99807472 | 66.9646223 |
| KLHL22 | 518.141314 | 671.90282 | 514.974002 | 684.583925 | 19.4517919 | 56.5148087 |
| FRRS1 | 320.517397 | 190.641987 | 320.526505 | 189.62731 | 54.8534528 | 17.5280613 |
| ZC2HC1A | 108.887813 | 161.012634 | 110.232831 | 162.803063 | 9.36441237 | 10.9943222 |
| SEC31A | 5066.64312 | 6319.61114 | 5087.25642 | 6265.11843 | 406.179764 | 203.327709 |
| BNIP2 | 3722.48704 | 2633.20094 | 3747.93688 | 2693.08429 | 166.200056 | 349.620045 |
| PGRMC2 | 2064.47283 | 3096.80344 | 2085.56708 | 3019.50904 | 190.686512 | 433.029602 |
| TSC2 | 2145.37104 | 2659.73397 | 2137.88696 | 2661.59705 | 165.668802 | 85.8442934 |
| DGUOK | 1150.74159 | 907.717466 | 1166.25052 | 893.152241 | 56.0419778 | 71.0969205 |
| CDV3 | 7801.06047 | 10284.4214 | 7867.89217 | 10270.1397 | 319.783423 | 1069.70131 |
| RTN4 | 14841.3052 | 17014.4108 | 14723.0982 | 16971.8017 | 353.541755 | 786.384906 |
| PPIL3 | 938.138645 | 594.562244 | 934.179446 | 592.057147 | 172.622051 | 12.3703783 |
| ATP2B4 | 2009.19508 | 2465.21792 | 2034.00295 | 2431.16019 | 129.284994 | 115.070464 |
| ABTB2 | 634.294888 | 1084.00132 | 634.350161 | 1075.32484 | 53.1412317 | 227.63826 |
| CHMP3 | 2028.81564 | 2468.06882 | 2019.78618 | 2460.34643 | 108.837219 | 136.914175 |
| RIN1 | 1331.35633 | 695.437982 | 1329.34538 | 696.448882 | 57.0639629 | 193.808945 |
| KRAS | 1309.83605 | 1624.79679 | 1301.80281 | 1599.3984 | 27.3903932 | 132.617701 |
| IRF5 | 253.78936 | 333.018264 | 254.793374 | 331.189143 | 10.1375969 | 25.2960162 |
| MBOAT2 | 865.062366 | 1183.17994 | 862.430015 | 1185.2549 | 19.7746379 | 147.793916 |
| B3GNT2 | 999.568696 | 753.22917 | 964.454567 | 763.246403 | 83.474547 | 57.0015636 |
| AL118516.1 | 167.404135 | 98.5636462 | 163.573392 | 100.108779 | 26.1409467 | 12.7101565 |
| TMEM9 | 1670.52582 | 2718.97809 | 1674.76881 | 2644.37832 | 46.3268905 | 566.173802 |
| ZNFX1 | 2357.53922 | 3407.89041 | 2393.81633 | 3382.47611 | 140.542061 | 493.512334 |
| BOLA1 | 531.167304 | 417.021986 | 537.223741 | 418.627292 | 28.4660909 | 29.2342829 |
| BCL2L1 | 5049.51415 | 3816.70671 | 5042.45723 | 3854.44485 | 210.276385 | 419.68867 |
| TPGS2 | 2313.48856 | 2812.44879 | 2311.82093 | 2807.41705 | 45.2047089 | 212.015238 |
| DUSP16 | 1089.1088 | 1487.46765 | 1089.55122 | 1492.40465 | 101.809537 | 136.146651 |
| COPA | 10620.6172 | 11987.9689 | 10712.2631 | 11924.8874 | 266.587185 | 483.889926 |
| UAP1 | 3600.84735 | 2729.6483 | 3694.44644 | 2751.48627 | 242.255819 | 258.85503 |
| NIF3L1 | 1242.31002 | 913.028733 | 1211.87082 | 910.530654 | 137.210971 | 59.8395622 |
| TFB1M | 386.81889 | 299.859735 | 391.098865 | 290.235268 | 10.6395516 | 28.7778415 |
| PRTFDC1 | 1865.95855 | 1506.52319 | 1873.78178 | 1487.44779 | 131.761709 | 75.4411302 |
| DNMBP | 3303.566 | 1795.54021 | 3197.58762 | 1762.34278 | 744.069789 | 283.674498 |
| ROS1 | 22.0325507 | 65.4377267 | 22.7549442 | 64.3595151 | 4.66978755 | 24.0933283 |
| WNT9A | 365.380408 | 613.872291 | 364.495502 | 605.760671 | 9.67971546 | 134.592844 |
| COL18A1 | 645.959793 | 1291.78177 | 634.59373 | 1290.26669 | 74.3931525 | 371.784616 |
| CYB561D1 | 344.511939 | 447.189022 | 343.01154 | 444.464933 | 26.6193437 | 27.9716859 |
| LINC00973 | 147.809441 | 26.1969068 | 145.137537 | 23.2380979 | 80.9494847 | 14.3693408 |
| ANTXR2 | 931.290701 | 406.619631 | 915.636853 | 387.001081 | 176.815283 | 134.761731 |
| ASAH2B | 303.038975 | 207.881549 | 306.79087 | 205.860822 | 13.187332 | 32.304726 |
| HCN4 | 9.52311474 | 26.0327795 | 9.84722213 | 25.9429162 | 2.21975511 | 5.49615745 |
| ZFX | 1282.58565 | 1598.46271 | 1270.68554 | 1571.66012 | 101.463583 | 74.4667657 |
| ARHGAP26 | 3556.62109 | 1121.23727 | 3552.62111 | 1082.81871 | 1768.6055 | 224.427884 |
| USP33 | 2378.78955 | 1903.5047 | 2366.76732 | 1873.06401 | 112.099376 | 156.694943 |
| SMARCC1 | 7285.66642 | 7939.98288 | 7300.71857 | 7971.19611 | 190.225039 | 145.764221 |
| MT-ND6 | 15460.762 | 19557.1341 | 15658.193 | 19719.9185 | 853.840474 | 1666.32389 |
| TGM1 | 42.419969 | 165.448932 | 41.5007657 | 161.745062 | 11.0190885 | 88.4512563 |
| CHST15 | 2055.25694 | 2503.92015 | 2073.10514 | 2506.18694 | 136.924638 | 122.999071 |
| LINC02535 | 102.932045 | 28.8895766 | 97.7971287 | 26.847161 | 38.2333095 | 12.6396774 |
| TIMP1 | 2294.34275 | 1423.48013 | 2279.61906 | 1412.08422 | 468.623677 | 100.599606 |
| BTBD2 | 1659.30951 | 1949.07858 | 1655.59176 | 1922.51747 | 52.3502324 | 111.920724 |
| KIAA0040 | 806.446617 | 479.200413 | 776.739196 | 464.154767 | 131.217967 | 82.4881449 |
| CTNNB1 | 10647.9319 | 9355.32536 | 10566.3632 | 9315.59769 | 318.198417 | 415.982191 |
| KIAA1522 | 6661.77519 | 9902.87575 | 6652.25552 | 9906.60673 | 1201.73691 | 34.476027 |
| MTHFD2 | 6894.55231 | 5729.18554 | 7026.98735 | 5701.13627 | 478.559287 | 209.801261 |
| TSPAN17 | 2224.43795 | 2697.50086 | 2177.36474 | 2700.61351 | 152.470164 | 128.296954 |
| MYO1B | 3889.59713 | 4683.96786 | 3900.95138 | 4652.4804 | 284.850546 | 143.581406 |
| LAMB2 | 5544.11621 | 10492.0628 | 5492.44951 | 10344.4067 | 1303.10424 | 2030.66345 |
| TAF1A | 156.660636 | 105.797813 | 152.711077 | 106.827412 | 13.8604551 | 12.6570725 |
| SNRNP70 | 6274.73812 | 5696.70351 | 6285.8443 | 5677.47803 | 150.450677 | 180.162923 |
| IRF2BP1 | 1221.72263 | 998.598362 | 1230.81103 | 968.294626 | 36.8042721 | 86.869054 |
| SF3A3 | 4978.9846 | 4435.63201 | 5012.20329 | 4468.8459 | 170.794194 | 136.507724 |
| IL18R1 | 128.075151 | 84.9895004 | 131.189031 | 83.3641056 | 15.9435359 | 5.36307165 |
| CASC19 | 232.633048 | 58.5660812 | 229.538291 | 60.5412867 | 27.6631872 | 36.7460932 |
| VSIR | 498.670613 | 1051.34959 | 481.373369 | 1027.73606 | 98.6948142 | 310.814884 |
| MED15 | 2515.67937 | 2901.98846 | 2515.39069 | 2908.30994 | 80.6035535 | 141.588593 |
| AFF1 | 2453.04562 | 3276.92108 | 2435.17316 | 3236.24522 | 299.844066 | 162.490258 |
| AC022211.2 | 150.60785 | 101.393308 | 151.090096 | 98.2715795 | 17.2971443 | 8.9356903 |
| HSP90AA1 | 128397.753 | 99479.0147 | 125699.157 | 99183.1038 | 9771.34941 | 8905.24683 |
| S100A10 | 15680.7046 | 20645.7035 | 15666.3635 | 20798.7871 | 1801.39725 | 1065.66128 |
| SOD1 | 7258.71507 | 8194.80748 | 7239.67802 | 8247.02629 | 220.262021 | 331.319385 |
| HECTD4 | 1800.29175 | 2149.05922 | 1789.59469 | 2155.10865 | 89.5017503 | 122.002721 |
| AP3D1 | 7133.02884 | 8358.46634 | 7123.26731 | 8426.90607 | 128.346117 | 550.71033 |
| GPR37L1 | 1.94843711 | 10.9283485 | 1.33134169 | 10.6787167 | 2.16973646 | 2.14461002 |
| NUP205 | 6541.24689 | 5908.06181 | 6556.52369 | 5872.52483 | 192.633864 | 175.267557 |
| NF1 | 4087.02238 | 3423.21561 | 4103.78418 | 3422.49941 | 175.577994 | 221.235537 |
| GALE | 3018.46201 | 2248.95896 | 3004.18698 | 2277.39516 | 314.800391 | 193.051351 |
| GNAI3 | 3803.58259 | 3511.29963 | 3781.8391 | 3501.62128 | 60.2564231 | 80.9558355 |
| SESTD1 | 786.996113 | 652.230018 | 774.158778 | 652.10633 | 43.4060669 | 30.9659606 |
| NUDCD3 | 3691.24063 | 4274.61321 | 3727.32001 | 4279.15764 | 204.473716 | 129.580453 |
| PDHB | 1815.52404 | 1487.10882 | 1832.9375 | 1481.02879 | 154.236077 | 25.1392443 |
| TXNRD2 | 816.353542 | 958.894437 | 816.152066 | 953.269458 | 45.2580898 | 30.4130957 |
| RALBP1 | 3597.13603 | 4013.37738 | 3580.42528 | 3987.69523 | 93.6762143 | 143.843144 |
| RAB6A | 3509.24429 | 3866.81853 | 3501.64047 | 3914.57833 | 53.5159047 | 137.943255 |
| SLC1A4 | 1515.61796 | 2163.87661 | 1539.62236 | 2198.00499 | 213.835225 | 192.212437 |
| ZBTB5 | 420.923435 | 553.252963 | 418.464721 | 547.224007 | 18.1530683 | 61.0034625 |
| ZBTB4 | 2174.72171 | 2872.29765 | 2208.9942 | 2843.21483 | 142.122848 | 318.553878 |
| NPAS2 | 1337.08599 | 761.036644 | 1335.12055 | 760.192373 | 93.6638321 | 201.960674 |
| COX8A | 1933.75647 | 1479.67791 | 1922.00835 | 1488.01363 | 215.392465 | 76.9435187 |
| PSAPL1 | 1.43122404 | 14.286369 | 1.26443664 | 12.6385168 | 1.41571073 | 7.75460323 |
| MKNK2 | 2698.18731 | 3309.33674 | 2688.9544 | 3274.10385 | 207.575538 | 175.436178 |
| WDR19 | 291.605401 | 368.404441 | 294.016595 | 378.73856 | 12.722983 | 27.8570976 |
| DYRK1A | 2194.07151 | 2613.02578 | 2226.62969 | 2591.38695 | 72.2255364 | 182.024603 |
| SPATA33 | 344.763681 | 446.325807 | 340.349873 | 434.851328 | 34.9419112 | 24.0973751 |
| GARNL3 | 88.7324089 | 180.142323 | 88.9065046 | 174.022413 | 13.5075665 | 52.3467289 |
| USP39 | 3335.27274 | 2814.33634 | 3389.42506 | 2818.36405 | 208.215681 | 115.836562 |
| DCLRE1C | 1005.72717 | 837.017251 | 999.469757 | 835.837348 | 63.71094 | 35.2345891 |
| B3GNTL1 | 232.324852 | 151.686005 | 235.144371 | 153.736802 | 33.6757627 | 18.9359873 |
| AC137932.2 | 34.8658564 | 9.43668054 | 32.4432935 | 8.54097158 | 14.5055412 | 4.33883223 |
| VDR | 2835.91914 | 1337.41286 | 2823.8197 | 1326.59643 | 1040.46803 | 100.194003 |
| PRDX6 | 9308.77666 | 13692.0552 | 9336.63249 | 13813.2844 | 391.767078 | 2504.04406 |
| FOPNL | 2880.83713 | 2286.84767 | 2881.39554 | 2307.56746 | 114.051254 | 229.433878 |
| NCAPD3 | 3287.84156 | 2719.45887 | 3228.9477 | 2731.90462 | 154.685783 | 198.797433 |
| SLC26A4-AS1 | 30.1496306 | 9.60265809 | 29.5109919 | 9.34389639 | 9.33402179 | 3.81212068 |
| RAPGEF1 | 3376.3523 | 4299.82096 | 3388.03453 | 4264.01101 | 290.712339 | 322.792348 |
| SLC19A3 | 14.7584759 | 68.000296 | 14.6021863 | 67.2459979 | 6.47961241 | 38.3103212 |
| SLC16A4 | 218.056138 | 321.382302 | 212.056777 | 306.371864 | 32.4834357 | 32.5567029 |
| DIDO1 | 3687.89443 | 4387.15872 | 3654.97862 | 4278.51315 | 141.660328 | 317.249294 |
| NUDT22 | 527.909757 | 428.593312 | 528.390116 | 433.165642 | 25.4931282 | 31.3068168 |
| SLC41A2 | 520.631379 | 970.175361 | 494.438446 | 962.963569 | 136.770624 | 167.497248 |
| EVC | 392.293213 | 615.53586 | 390.304677 | 619.178532 | 83.3526097 | 36.8428596 |
| FOXO3 | 1150.55748 | 1347.2985 | 1156.25156 | 1375.60306 | 34.6829139 | 80.9964659 |
| ZSWIM6 | 962.919518 | 1141.82989 | 973.517772 | 1136.48023 | 53.674201 | 53.4172445 |
| TMEM208 | 1197.21583 | 1553.04019 | 1198.75996 | 1552.38247 | 20.1822938 | 189.520798 |
| FLT4 | 260.764692 | 171.746462 | 246.969178 | 173.354502 | 41.7967075 | 17.5230097 |
| HLA-DMA | 102.62548 | 185.671704 | 98.8829147 | 173.046264 | 16.6019184 | 43.7792003 |
| TLR3 | 99.8593107 | 190.59513 | 98.8521417 | 185.676684 | 17.9485493 | 47.1057778 |
| ZBTB47 | 290.532313 | 421.158295 | 284.739729 | 431.020128 | 30.7193264 | 59.1528292 |
| MCM2 | 3947.39125 | 4419.87485 | 4013.15774 | 4427.40426 | 152.205547 | 134.211806 |
| AC112220.2 | 130.394655 | 195.251411 | 129.720287 | 194.139511 | 18.3768567 | 21.9310547 |
| NEK3 | 215.954665 | 134.079803 | 215.88798 | 138.239261 | 42.7064275 | 10.1220851 |
| PGLS | 754.474238 | 1034.75616 | 751.170382 | 1039.87073 | 93.0878559 | 93.0891215 |
| GON7 | 393.11363 | 292.608724 | 388.139509 | 303.187019 | 29.304356 | 32.7477157 |
| FBXW4 | 1026.35792 | 1295.22817 | 1041.56466 | 1277.12803 | 87.3941759 | 90.3785404 |
| ARHGEF37 | 215.397571 | 393.244097 | 225.683757 | 372.4242 | 52.1698951 | 69.4090579 |
| ADAMTS15 | 127.971654 | 188.50357 | 120.17771 | 197.112667 | 19.0489902 | 15.8423931 |
| RASA1 | 1470.50883 | 1229.63344 | 1473.15677 | 1243.59508 | 31.2313384 | 96.1627507 |
| HPS1 | 1047.46095 | 1391.2335 | 1045.70756 | 1369.42647 | 15.2877301 | 190.614711 |
| SAMD1 | 1802.41926 | 2115.59192 | 1801.8656 | 2174.43328 | 26.0203372 | 148.149901 |
| TSC22D2 | 902.593856 | 1056.87013 | 898.263763 | 1042.27027 | 47.0009176 | 43.8491252 |
| PSG9 | 2.45500828 | 14.5029272 | 2.64449526 | 15.1926455 | 1.29645513 | 7.6969241 |
| EFCAB14 | 3633.24639 | 3951.66364 | 3658.1918 | 3931.04984 | 99.7668905 | 78.0711283 |
| GNPTG | 542.114324 | 858.27563 | 555.00304 | 341.748089 | 59.656253 | 173.828555 |
| SNX9 | 2197.22165 | 1939.05398 | 2215.9374 | 1920.85343 | 79.109324 | 82.3097561 |
| PGPEP1 | 627.584626 | 458.835257 | 610.009626 | 458.520153 | 68.2494249 | 49.9991324 |
| TTC3 | 7541.40186 | 9832.56253 | 7588.83135 | 9986.58648 | 805.605667 | 753.050836 |
| POLR2H | 2120.36666 | 1669.78003 | 2127.87718 | 1664.04849 | 179.588956 | 131.522322 |
| SPTBN5 | 98.043345 | 163.286063 | 97.4240939 | 166.590851 | 10.3834917 | 35.1880988 |
| CHMP4B | 3503.49715 | 3939.96762 | 3496.31595 | 3927.83101 | 137.86935 | 140.009419 |
| AP001053.1 | 10.1551297 | 39.6485798 | 10.0034039 | 38.5936416 | 3.15027417 | 21.7478726 |
| TRIM26 | 1950.85043 | 2182.51715 | 1969.18798 | 2177.34309 | 85.018137 | 37.5315908 |
| MBOAT7 | 3614.99498 | 4493.1943 | 3575.77485 | 4543.93353 | 86.9376666 | 461.122537 |
| CAT | 1201.64264 | 1454.46519 | 1192.15048 | 1431.87895 | 40.8262132 | 120.121969 |
| CCDC144NL-AS1 | 1.88258309 | 11.3832987 | 1.60685514 | 12.0989534 | 2.09068549 | 3.84027569 |
| SGMS2 | 889.745744 | 1028.8758 | 888.107599 | 1025.41317 | 33.0327548 | 49.1437781 |
| EBAG9 | 842.510181 | 712.492727 | 840.658611 | 714.445958 | 44.0272397 | 35.6323563 |
| FAM86DP | 342.67736 | 200.965584 | 335.793609 | 194.483251 | 79.4604343 | 26.3665731 |
| AC007773.1 | 39.6062741 | 19.6672779 | 40.4716184 | 21.8157182 | 7.45692891 | 4.22247548 |
| TCF25 | 2422.16381 | 3218.41906 | 2389.71167 | 3154.90154 | 299.526591 | 234.603478 |
| RICTOR | 1942.00591 | 1427.36314 | 1968.14258 | 1412.61778 | 160.117036 | 190.586054 |
| INPPL1 | 4695.25848 | 5627.36107 | 4699.10375 | 5652.53172 | 207.36853 | 428.992141 |
| AL035252.3 | 54.4784042 | 24.7336234 | 55.3182098 | 23.4799162 | 6.00490921 | 9.95613546 |
| ELOF1 | 1128.64422 | 957.328804 | 1130.22757 | 956.883084 | 77.2134291 | 18.5351098 |
| PSMB1 | 5084.53447 | 4655.36884 | 5070.99346 | 4644.03648 | 103.728611 | 152.015507 |
| MEGF6 | 173.649334 | 329.159612 | 165.987414 | 322.48788 | 28.489798 | 93.4582146 |
| SNRPD2 | 6060.54469 | 5226.31238 | 6085.40134 | 5170.69539 | 331.318783 | 236.888927 |
| HSP90AA2P | 77.2486739 | 50.1245618 | 77.025252 | 45.5915114 | 5.38779378 | 8.57654221 |
| SORCS2 | 2.15510806 | 9.9677929 | 1.95632418 | 11.0827875 | 0.76807301 | 2.9099579 |
| ETV1 | 1675.29116 | 235.889477 | 1658.30998 | 229.254606 | 1210.95029 | 174.469677 |
| PUM1 | 2786.83609 | 3160.79748 | 2760.70234 | 3167.67508 | 149.8418 | 63.0188578 |
| ECT2 | 7907.26625 | 5213.66234 | 7850.14081 | 5225.09301 | 1430.98151 | 650.660283 |
| TCF3 | 3050.3585 | 3645.09264 | 3054.71616 | 3702.16084 | 189.393986 | 220.554094 |
| ABHD4 | 5044.3018 | 8451.68525 | 5062.13396 | 8515.63443 | 802.979134 | 1903.25437 |
| PHLDA1 | 2843.16374 | 818.559002 | 2638.19502 | 797.844746 | 1752.49829 | 219.482992 |
| AKR1A1 | 1744.71442 | 2103.35541 | 1743.45357 | 2107.65974 | 74.6355857 | 170.373608 |
| SLC38A7 | 1027.01754 | 1332.16 | 1020.6096 | 1313.23092 | 115.209396 | 90.9112503 |
| TMEM59 | 3089.39644 | 4042.37072 | 3103.53504 | 4022.89015 | 126.234537 | 527.668377 |
| BRCC3 | 1494.57671 | 1751.00565 | 1494.27329 | 1742.13135 | 40.762243 | 121.800468 |
| WFDC21P | 28.8310362 | 83.3168797 | 26.9576736 | 80.6475002 | 15.8583312 | 11.6949438 |
| IL1RL1 | 8.18376086 | 0.58973378 | 8.49132183 | 0.5188989 | 2.69369192 | 0.65948107 |
| PIP5K1C | 1048.9482 | 1274.48216 | 1066.08253 | 1265.83228 | 89.3421363 | 54.240943 |
| TATDN3 | 523.603308 | 434.541131 | 517.904047 | 437.981838 | 24.4009154 | 28.826897 |
| BCKDHB | 487.536365 | 597.819398 | 483.623863 | 602.937707 | 20.3520041 | 48.9224595 |
| HSPA1A | 488.339653 | 656.162674 | 481.804296 | 660.069537 | 12.4703998 | 92.789119 |
| WDR41 | 1625.46765 | 1912.61985 | 1622.88242 | 1909.03482 | 95.4914876 | 101.286962 |
| PHPT1 | 1317.38484 | 1724.12635 | 1312.92873 | 1725.68793 | 180.677866 | 30.3943511 |
| FANCM | 642.889838 | 524.66976 | 645.441098 | 516.107291 | 27.6522822 | 45.5101327 |
| AL645608.7 | 21.5503095 | 39.103233 | 22.5974843 | 38.6431717 | 4.87427205 | 2.8374495 |
| SHTN1 | 4315.8991 | 5308.05628 | 4298.90539 | 5314.3241 | 355.901907 | 340.067901 |
| TMEM135 | 1300.93493 | 948.51194 | 1261.83788 | 896.83849 | 142.754123 | 117.529627 |
| CCT8 | 10077.2493 | 8304.47947 | 10126.8918 | 8220.75214 | 713.593387 | 565.98006 |
| FAM13B | 871.950389 | 1401.55463 | 865.292619 | 1393.33555 | 90.4441102 | 324.634005 |
| SORBS3 | 2061.73404 | 2315.12441 | 2028.38235 | 2306.96416 | 85.1707369 | 69.9450689 |
| RCN3 | 18.9858836 | 7.75887119 | 19.182579 | 7.18288427 | 3.51660396 | 1.62351309 |
| OLMALINC | 778.537385 | 360.102211 | 785.942457 | 356.661664 | 206.883115 | 117.134293 |
| BCL2L12 | 1008.34695 | 834.759656 | 1005.52961 | 819.78521 | 41.2567451 | 68.3637888 |
| THAP2 | 102.035999 | 70.566779 | 103.501611 | 69.6489656 | 9.68546353 | 6.02506367 |
| PPDPF | 5641.15115 | 7234.33928 | 5623.10585 | 7149.96642 | 188.168409 | 908.522699 |
| SHISAL1 | 40.1434348 | 20.9812756 | 39.7527055 | 20.0374353 | 3.97506022 | 6.70095012 |
| RNF214 | 518.417191 | 429.655508 | 508.050931 | 414.677478 | 21.4515384 | 31.5584339 |
| ST6GALNAC2 | 17.4143597 | 38.0152053 | 15.875202 | 38.2495724 | 6.95858114 | 4.8806195 |
| DGAT2 | 258.565307 | 390.700083 | 258.084039 | 407.020251 | 15.5992741 | 76.7695483 |
| AADAC | 180.838919 | 52.1325473 | 170.437282 | 51.2169437 | 109.83265 | 12.4515268 |
| MUC1 | 1821.31907 | 4261.63732 | 1788.63128 | 4168.06426 | 451.767144 | 1757.95421 |
| TLNRD1 | 1817.10294 | 1497.76664 | 1853.89827 | 1467.16278 | 109.305547 | 124.400427 |
| AGRN | 17362.1252 | 23527.025 | 17370.1659 | 23554.0945 | 2552.07409 | 1733.67356 |
| MT-ND4 | 121329.104 | 144814.305 | 122477.79 | 144856.909 | 6217.25088 | 11069.3987 |
| STT3B | 6042.57546 | 6969.22259 | 5938.15424 | 7055.12272 | 388.127311 | 240.340146 |
| MED29 | 1732.24629 | 2019.67146 | 1715.05501 | 2028.7871 | 94.5875502 | 103.921461 |
| C3orf52 | 276.283077 | 341.113733 | 270.906118 | 334.519953 | 22.97835 | 16.5567034 |
| AL592071.1 | 17.662168 | 6.87611615 | 18.2764267 | 6.66398537 | 3.76062263 | 1.84059687 |
| INPP5F | 919.902197 | 664.933271 | 945.268048 | 657.793955 | 134.470948 | 58.4511063 |
| CYB5R1 | 980.190253 | 1184.48528 | 971.701496 | 1196.77542 | 84.1834627 | 49.7089016 |
| SNRPA | 2386.89616 | 2084.40099 | 2433.48669 | 2078.27697 | 117.047716 | 95.133096 |
| NKRF | 581.692086 | 497.692303 | 581.44088 | 507.445048 | 19.9330493 | 25.92971 |
| LINC01589 | 57.4279801 | 33.7976979 | 54.0324555 | 31.5246927 | 6.78826992 | 7.90634515 |
| KLF11 | 1144.02248 | 1545.27216 | 1138.04832 | 1544.55276 | 171.124984 | 96.268209 |
| PCSK7 | 586.007332 | 885.519874 | 588.231301 | 877.079142 | 111.806919 | 107.103673 |
| DTX3L | 1309.78623 | 1802.29489 | 1317.92255 | 1833.83523 | 154.071318 | 226.108209 |
| TUBA4A | 11401.1022 | 16699.1417 | 11561.0042 | 16819.9101 | 1804.29295 | 2340.31927 |
| DUSP3 | 3339.03106 | 4513.18869 | 3314.46016 | 4518.39266 | 417.528106 | 477.160815 |
| GSTO1 | 3279.82136 | 2567.36879 | 3284.73947 | 2513.32544 | 345.03237 | 198.191293 |
| MB | 48.78776 | 141.748712 | 45.9325031 | 143.071198 | 27.188921 | 28.6212859 |
| LIMS2 | 6.71886043 | 19.0189452 | 6.99199332 | 18.6149831 | 1.88528193 | 6.71027736 |
| STAM | 1958.15265 | 1653.9974 | 1948.70543 | 1657.79909 | 138.920878 | 77.9454063 |
| CEP41 | 426.736691 | 335.493533 | 418.194271 | 328.702338 | 21.4016345 | 36.5195171 |
| C1orf52 | 468.831569 | 367.822341 | 471.283628 | 351.409917 | 37.4762155 | 33.8201358 |
| COL4A4 | 2681.8415 | 5247.71116 | 2659.19336 | 5118.60018 | 559.352847 | 1719.95678 |
| PRRC2B | 4968.49316 | 5836.35486 | 4963.88359 | 5817.19714 | 337.887806 | 289.369001 |
| LZTR1 | 258.54467 | 368.669472 | 264.441362 | 353.102768 | 22.8193353 | 61.9075014 |
| HABP4 | 294.594393 | 453.690147 | 298.433286 | 446.105091 | 25.6175688 | 98.4994601 |
| VGF | 139.632407 | 32.3066619 | 141.553162 | 31.3570499 | 73.3086092 | 20.2614056 |
| CD151 | 4796.05205 | 6720.36417 | 4683.66623 | 6839.02213 | 762.746535 | 683.871417 |
| ARHGAP19 | 1187.8101 | 870.108806 | 1224.42486 | 879.54841 | 124.070519 | 116.309916 |
| AC092868.1 | 44.6188503 | 20.4541088 | 45.4640875 | 20.1747733 | 11.7526157 | 6.19914696 |
| AC016065.1 | 149.294652 | 107.953728 | 148.277457 | 100.854841 | 10.8237617 | 15.270368 |
| BEST3 | 17.8623213 | 6.65954864 | 18.7346078 | 7.18288427 | 4.34831317 | 2.7187924 |
| PRUNE2 | 9.86050571 | 24.712474 | 10.1945707 | 21.8739967 | 3.81478263 | 7.26931048 |
| TGM2 | 34702.288 | 12426.5609 | 34409.2617 | 12325.5167 | 7129.74602 | 6970.76752 |
| MEIS3 | 101.908357 | 147.700512 | 102.538764 | 151.50554 | 12.4901561 | 18.9425647 |
| BAG3 | 3766.69987 | 3179.57785 | 3735.0095 | 3157.14261 | 102.696401 | 278.966446 |
| AC100861.1 | 168.596553 | 121.05404 | 169.155504 | 123.952925 | 13.4258051 | 17.4633081 |
| INCENP | 2535.61472 | 2269.4061 | 2509.37319 | 2223.00002 | 80.58616 | 107.211848 |
| SEPTIN8 | 2424.89881 | 2908.71121 | 2412.47105 | 2877.38845 | 194.496272 | 159.228774 |
| AC008443.1 | 95.6627469 | 60.1995508 | 93.0498234 | 59.0375346 | 15.541199 | 9.89671604 |
| APOO | 728.949392 | 575.051871 | 724.541812 | 589.758823 | 39.159083 | 66.637781 |
| CCDC68 | 834.10008 | 424.294496 | 845.834489 | 422.786263 | 280.451321 | 81.5339631 |
| POLR2C | 3581.73158 | 3002.3367 | 3565.11147 | 3082.75063 | 193.787921 | 228.122511 |
| ORC2 | 1469.1385 | 1165.14614 | 1443.31648 | 1146.82739 | 150.73725 | 84.8517168 |
| ARHGAP12 | 3101.4852 | 1983.98293 | 3126.55759 | 1989.55462 | 723.320605 | 205.955136 |
| PIEZO1 | 6500.62221 | 9294.99869 | 6471.35161 | 9213.43606 | 1309.61011 | 244.03999 |
| ANAPC10 | 109.428352 | 73.851859 | 112.760439 | 74.1145208 | 16.4473317 | 4.24809594 |
| FSTL3 | 2658.11157 | 4978.54957 | 2644.31426 | 4977.81178 | 203.465823 | 1774.31829 |
| COX5B | 2417.40991 | 2742.71795 | 2422.85079 | 2695.0593 | 51.9375865 | 170.819761 |
| RARB | 188.014124 | 243.459096 | 184.291229 | 240.1008 | 15.7933685 | 18.8292928 |
| NFS1 | 1024.12291 | 871.786931 | 1027.53666 | 890.615489 | 54.8764625 | 52.434659 |
| COIL | 1248.69623 | 1048.73454 | 1220.15512 | 1050.86511 | 101.721057 | 40.501511 |
| CASP1 | 11.4092341 | 35.2616236 | 11.4625197 | 28.2410048 | 4.7919847 | 17.1545199 |
| MPZL1 | 8315.47518 | 8935.09059 | 8366.04738 | 8863.28698 | 197.466827 | 229.189353 |
| COA6-AS1 | 41.0588874 | 22.6812516 | 40.1950933 | 21.1603737 | 8.37743356 | 3.58886106 |
| BTBD8 | 125.940555 | 79.7442792 | 119.584825 | 82.8431573 | 16.7855498 | 16.4745834 |
| ABCA1 | 821.371435 | 635.924588 | 836.339638 | 638.843676 | 60.4031115 | 74.5443368 |
| TULP4 | 920.592642 | 1093.53875 | 915.733572 | 1098.1583 | 55.1244166 | 72.6165819 |
| LLGL1 | 1639.90665 | 1857.26359 | 1642.06333 | 1903.2414 | 46.5493572 | 99.1415202 |
| SLC8A1 | 87.8563313 | 42.8640336 | 82.2051286 | 42.3354077 | 31.8007844 | 5.14540466 |
| ZNF385A | 592.073786 | 762.636511 | 598.674713 | 765.438617 | 76.9115473 | 31.6905741 |
| RUFY1 | 1169.68012 | 1311.10911 | 1179.20184 | 1310.83233 | 50.5171066 | 38.9216543 |
| SLC39A13 | 467.927937 | 597.785292 | 468.231858 | 580.189735 | 22.5771748 | 71.5074255 |
| ST3GAL5 | 1408.13775 | 488.000836 | 1382.76208 | 494.437406 | 836.472879 | 75.8066128 |
| BCL2L11 | 531.625614 | 681.775981 | 542.698026 | 663.148668 | 40.2459671 | 75.8174676 |
| RLF | 1054.12401 | 848.047397 | 1087.4139 | 834.267884 | 85.2250964 | 71.697776 |
| PAM | 3392.94219 | 5378.68562 | 3336.17103 | 5367.72818 | 504.75502 | 1216.0236 |
| RNF19A | 814.130663 | 1146.96335 | 822.95799 | 1130.11698 | 142.714848 | 102.514529 |
| PPARA | 578.0588 | 427.757637 | 576.021592 | 428.019839 | 26.069644 | 69.5424505 |
| SLC25A11 | 2034.17099 | 1803.67552 | 2038.68998 | 1784.07551 | 93.2327914 | 68.3732752 |
| GK5 | 1042.23867 | 875.284556 | 1043.04174 | 879.671254 | 78.8867926 | 40.1802234 |
| ATF1 | 845.417037 | 642.877737 | 840.992767 | 650.849354 | 32.0988854 | 95.9739929 |
| CLDN3 | 117.820334 | 176.635158 | 115.885971 | 172.68883 | 13.431701 | 33.074456 |
| HSP90AB1 | 80869.7437 | 72325.5092 | 82266.5488 | 72020.7345 | 4330.92693 | 1866.9153 |
| AC125807.2 | 483.703179 | 626.255576 | 486.782217 | 611.398454 | 56.8590071 | 47.3653965 |
| GRID1 | 68.6082399 | 117.847557 | 71.4891315 | 104.764206 | 7.58766914 | 32.5157861 |
| MAGI2 | 157.991236 | 116.800968 | 154.534709 | 118.703027 | 8.07107388 | 15.9233654 |
| SHKBP1 | 1590.43339 | 1945.61161 | 1579.7723 | 1932.98593 | 69.92248 | 197.284771 |
| DGCR6L | 687.415563 | 837.463294 | 688.011335 | 812.979908 | 22.2724588 | 83.9369721 |
| TBX2 | 101.684376 | 67.2068672 | 100.431958 | 67.4836962 | 14.8951437 | 10.8867439 |
| NCOA5 | 1859.06416 | 2107.97954 | 1871.21969 | 2078.92829 | 82.3263882 | 101.238284 |
| SLC35G1 | 620.585808 | 508.691403 | 615.920687 | 505.935257 | 43.831875 | 38.3412679 |
| ZNF200 | 508.049907 | 407.443531 | 503.919045 | 396.538356 | 34.3954329 | 38.4464799 |
| LINC01119 | 16.3958417 | 5.7190447 | 17.8784646 | 6.45768215 | 5.74222666 | 1.753559 |
| AC017100.1 | 65.0197503 | 40.288341 | 65.6021492 | 40.2691583 | 5.48640557 | 9.35989667 |
| GRIP1 | 278.724558 | 362.629545 | 284.209724 | 362.470133 | 32.4679631 | 30.1583388 |
| SNRNP48 | 1086.7665 | 848.073103 | 1086.58106 | 857.440301 | 132.544328 | 54.3302731 |
| ARAP1 | 1918.40086 | 1639.28723 | 1944.5116 | 1616.6131 | 119.877902 | 96.3327822 |
| MT-ND4L | 14173.8805 | 16552.5216 | 14158.6312 | 16830.9688 | 587.458878 | 1249.31989 |
| LSR | 4655.97003 | 5242.24145 | 4671.51751 | 5149.31183 | 154.236859 | 294.742525 |
| EMP2 | 1089.42962 | 1566.37839 | 1100.0544 | 1562.30002 | 36.2461126 | 328.558207 |
| AFAP1 | 1424.08546 | 2202.62993 | 1431.961 | 2187.72454 | 49.3354954 | 566.792079 |
| CSMD3 | 76.5643232 | 23.2737419 | 72.794934 | 24.8139511 | 45.8088759 | 6.32335062 |
| GRN | 4372.94418 | 7078.37236 | 4382.57535 | 7007.52108 | 998.32646 | 1305.83418 |
| RPL29 | 14498.5122 | 13702.6711 | 14544.0693 | 13744.6459 | 293.972189 | 262.52685 |
| GMNN | 1937.14204 | 1657.51846 | 1927.10139 | 1620.07867 | 71.031984 | 133.582336 |
| PFKL | 4543.67457 | 5196.56103 | 4528.13627 | 5233.5085 | 231.122514 | 275.486731 |
| ST5 | 238.490504 | 292.48479 | 242.018726 | 291.94276 | 17.5125589 | 15.8954803 |
| TNFRSF21 | 3130.63171 | 2693.13456 | 3120.47141 | 2693.24439 | 242.327561 | 82.4545504 |
| AC108752.1 | 11.3732557 | 88.3696606 | 10.1552422 | 89.1252046 | 10.4369483 | 53.6747268 |
| GCNA | 45.011309 | 14.4079938 | 40.4237539 | 15.0007478 | 25.4669545 | 3.62574417 |
| CLDN2 | 45.6323697 | 11.4147005 | 45.7752798 | 12.2580441 | 29.6695348 | 4.27066694 |
| L1CAM | 490.380911 | 2023.02987 | 483.060142 | 1973.03066 | 261.850363 | 1396.23223 |
| PPP1R14C | 271.135553 | 340.543702 | 270.907727 | 343.42686 | 19.4847913 | 29.9339375 |
| BHLHE40 | 3863.08014 | 2208.22243 | 3758.14003 | 2184.55958 | 1242.04885 | 281.815927 |
| QRICH2 | 42.9349748 | 71.5546238 | 43.022586 | 63.856567 | 2.43126438 | 18.7823327 |
| GBA2 | 936.732042 | 769.899862 | 929.130233 | 769.435595 | 57.1746883 | 71.8866879 |
| KCNN4 | 891.570895 | 642.153227 | 905.623264 | 628.355697 | 52.0367029 | 121.89923 |
| SIRT7 | 971.701012 | 1176.11405 | 978.154143 | 1183.03345 | 85.8198141 | 73.6444319 |
| CAPZA1 | 8235.17593 | 6833.22298 | 8265.23005 | 6848.16666 | 444.667634 | 640.585047 |
| ZNRF3 | 722.674187 | 977.744996 | 713.358931 | 981.584535 | 47.9935189 | 160.830222 |
| GMEB1 | 708.521205 | 626.739658 | 702.983354 | 623.706424 | 22.0404255 | 29.7164734 |
| LIPA | 2912.80871 | 3609.13043 | 2942.78878 | 3646.1154 | 92.260059 | 431.829314 |
| KLC1 | 863.538393 | 1063.97211 | 855.864961 | 1107.51253 | 41.0513169 | 112.407448 |
| CDC27 | 4838.63159 | 4336.72328 | 4796.98056 | 4374.73302 | 128.840495 | 234.254613 |

Individual RNAseq data for H2122 (top) and H2030 (bottom) cells that were used to
generate the combined differentially expressed genes (DEG) for H2122 and H2030.

| | | | | | | |
|---|---|---|---|---|---|---|
| TNS4 | 19659.9784 | 8208.28816 | 19468.741 | 8109.78798 | 499.318088 | 203.502488 |
| KRT17 | 12943.4074 | 30135.8977 | 13101.7528 | 29491.8748 | 319.836725 | 1157.11336 |
| APOL1 | 1438.40035 | 5204.08531 | 1460.76043 | 5266.11119 | 42.9282518 | 378.328894 |
| DCLK1 | 1677.67062 | 278.656265 | 1654.29877 | 280.033207 | 48.1145505 | 12.3524582 |
| MCFD2 | 8911.97993 | 4064.09851 | 8916.13454 | 4067.37343 | 80.6370067 | 69.5322144 |
| MYEOV | 6204.51635 | 2209.59837 | 6276.71587 | 2150.37327 | 250.749227 | 105.984528 |
| SYNPO | 1421.42767 | 3868.17984 | 1415.3816 | 3828.87744 | 10.9927091 | 79.8888826 |
| MYH14 | 4649.21316 | 11935.5451 | 4707.5369 | 11560.6629 | 109.105283 | 729.247837 |
| MAP2 | 946.59105 | 3001.62761 | 932.636045 | 2976.28214 | 55.2535627 | 49.6632662 |
| ITGA2 | 5331.03531 | 1829.46908 | 5427.48691 | 1812.36353 | 326.589758 | 51.7737477 |
| PHLDA1 | 4070.31772 | 936.644303 | 4067.09686 | 943.356703 | 429.440177 | 77.3033116 |
| DMBT1 | 3707.44814 | 1156.4681 | 3736.40455 | 1123.14971 | 118.974014 | 88.9415535 |
| TRIM29 | 4803.15734 | 10302.2284 | 4854.06085 | 10170.2824 | 100.50011 | 276.317387 |
| ETV4 | 893.663292 | 41.2125366 | 894.92238 | 37.9986062 | 15.3623669 | 6.47241047 |
| CALB2 | 1350.88767 | 261.367841 | 1348.51318 | 257.908452 | 8.6245166 | 27.8360978 |
| UBASH3B | 1377.522 | 279.873408 | 1389.7487 | 271.77264 | 25.7968057 | 25.7989964 |
| ABHD2 | 9086.26489 | 17079.7928 | 9083.25493 | 17055.6823 | 122.268566 | 322.928073 |
| ARNT2 | 1646.24548 | 388.799969 | 1592.58291 | 394.720103 | 105.078906 | 13.3436791 |
| CTSL | 16887.6978 | 9118.13753 | 16873.1318 | 9122.31745 | 175.058006 | 192.818888 |
| AREG | 3732.00994 | 1491.95464 | 3721.22768 | 1474.42287 | 122.427873 | 36.1598296 |
| RGS2 | 2066.97853 | 590.625085 | 2089.64055 | 591.617953 | 67.0194542 | 50.898579 |
| EPHA2 | 5759.72442 | 2616.63955 | 5771.62737 | 2620.68265 | 156.620681 | 109.24428 |
| LCN2 | 1149.74764 | 2941.39702 | 1176.26 | 2925.73566 | 48.9161862 | 81.8792864 |
| B2M | 14055.2917 | 23897.7073 | 14090.291 | 24117.6756 | 124.604813 | 486.776642 |
| NCF2 | 772.452603 | 2123.65571 | 774.55922 | 2139.85157 | 11.3728536 | 66.7226922 |
| ETV5 | 611.982157 | 37.2332569 | 611.262411 | 38.1411773 | 7.20980643 | 5.40706793 |
| NEDD9 | 636.486664 | 1963.16232 | 639.707903 | 1953.26365 | 46.6847045 | 69.4749717 |
| FOSL1 | 2495.40556 | 869.618255 | 2471.4018 | 869.011604 | 94.9629124 | 73.0880376 |
| LTBP4 | 841.589692 | 2471.25718 | 819.19612 | 2449.25799 | 66.2146088 | 124.64483 |
| PSAP | 18135.3251 | 31147.455 | 18064.5042 | 31212.6352 | 328.691055 | 831.490359 |
| KRT18 | 30443.0987 | 16584.24 | 30646.9122 | 17003.5502 | 555.028768 | 747.915403 |
| SPRY4 | 575.751799 | 37.4479196 | 568.421797 | 36.051719 | 21.1453925 | 2.89062639 |
| BACE2 | 15345.5791 | 26564.3153 | 15322.3111 | 26879.8836 | 164.188914 | 859.062284 |
| BMF | 119.252311 | 801.781481 | 131.43973 | 799.622844 | 22.2108383 | 31.0405954 |
| EREG | 2999.78652 | 970.560867 | 2869.90697 | 974.572842 | 321.314579 | 41.0405912 |
| SAT1 | 2602.49361 | 1044.22727 | 2616.78424 | 1063.96097 | 56.5834343 | 59.7620573 |
| S100A9 | 3247.4969 | 1429.7839 | 3192.96715 | 1410.90477 | 101.573338 | 55.9839343 |
| LAMB2 | 4041.36988 | 8020.649 | 3995.38821 | 7939.69781 | 224.799344 | 149.201598 |
| MATN2 | 520.340221 | 1490.34025 | 511.997575 | 1491.03227 | 14.8449264 | 5.43608233 |
| IGFBP3 | 619.342355 | 1715.86828 | 620.362037 | 1747.91809 | 25.3486705 | 63.5597949 |
| UGT1A6 | 989.174343 | 2356.59117 | 983.244322 | 2398.82592 | 26.3950745 | 81.9256235 |
| SRI | 2322.50466 | 4637.48874 | 2313.17181 | 4625.91728 | 56.158354 | 173.155662 |
| EPGN | 2931.54661 | 1165.13271 | 2912.60395 | 1163.96352 | 228.02167 | 37.0350277 |
| PLXNA2 | 771.917707 | 1944.07387 | 782.624864 | 1925.47185 | 36.6614171 | 36.2425712 |
| EPB41L1 | 1553.88625 | 3218.5976 | 1550.23291 | 3234.01181 | 45.9515678 | 28.7336962 |
| KRT80 | 9642.08461 | 15647.2518 | 9644.49483 | 15731.2628 | 205.506224 | 224.218934 |
| ACSL5 | 585.016275 | 94.906049 | 585.098692 | 94.1704589 | 18.6431923 | 4.72548128 |
| SMOX | 7293.84306 | 3755.44584 | 7431.31062 | 3793.25217 | 422.223899 | 73.4404072 |
| ITGA6 | 5458.85048 | 3047.8017 | 5467.60796 | 3047.32301 | 41.5786052 | 78.2911682 |
| SLAMF9 | 1086.09881 | 305.256799 | 1092.18422 | 322.227188 | 41.6326783 | 32.2394553 |
| ETV | 525.492181 | 71.3050703 | 533.293681 | 71.179035 | 21.1509063 | 2.97901154 |
| AKR1C1 | 20879.8182 | 38953.9411 | 20251.1016 | 39048.6743 | 1550.70131 | 287.715831 |
| AQP3 | 3569.528 | 7160.36442 | 3441.87757 | 7147.4844 | 256.973644 | 197.805429 |
| MCAM | 255.712413 | 970.846697 | 264.357931 | 936.748249 | 18.8967557 | 68.3521657 |
| LCAL1 | 2228.80184 | 4366.80418 | 2242.01017 | 4279.98613 | 62.4160515 | 190.676789 |
| BCAS1 | 011.2712 | 2359.69736 | 993.693252 | 2359.07659 | 73.2425152 | 36.3143449 |
| LAPTM4A | 4433.13971 | 7702.65055 | 4356.15896 | 7753.36779 | 170.005027 | 104.742932 |
| ALDH3B1 | 4537.83403 | 8193.81833 | 4498.25267 | 8262.21869 | 79.6522655 | 405.453875 |
| AKR1C2 | 52897.9785 | 84400.5594 | 52420.3849 | 83691.7547 | 1928.60151 | 1767.20849 |
| PTGES | 15921.3191 | 9618.66864 | 15743.0555 | 9582.36204 | 632.051487 | 100.022107 |
| POLM | 1956.57012 | 825.164869 | 1916.89476 | 833.811553 | 71.0277371 | 45.4864634 |
| LGALS3BP | 5734.64475 | 9813.59605 | 5663.75534 | 9748.75459 | 222.005713 | 225.753025 |
| DAPK1 | 1098.45307 | 2352.44194 | 1097.75659 | 2383.16598 | 17.1550314 | 92.4647593 |
| KRT4 | 24.1630616 | 442.77452 | 22.289474 | 482.538393 | 4.50438805 | 76.0308489 |
| LDLR | 3843.34821 | 2083.71178 | 3836.84713 | 2052.75079 | 27.8860851 | 73.3548046 |
| CD36 | 19248.3921 | 31248.1982 | 19244.6183 | 31443.0206 | 270.862053 | 1359.88267 |
| BMP2 | 2551.00926 | 4808.01312 | 2518.71056 | 4775.46617 | 116.124095 | 162.82888 |
| THBS1 | 2545.67523 | 5354.6638 | 2569.39191 | 5487.06869 | 196.605012 | 294.929327 |
| DUSP6 | 394.746093 | 16.170091 | 401.016755 | 15.7825561 | 36.7505834 | 3.98715878 |
| ANXA8 | 249.190414 | 859.462841 | 249.642108 | 841.736328 | 19.5768451 | 31.1629498 |
| ABLIM1 | 12112.1385 | 18093.2852 | 12106.6875 | 18003.6738 | 53.1252866 | 188.953445 |
| DOCK4 | 892.292599 | 281.892163 | 889.350011 | 280.140372 | 13.1680247 | 9.4656715 |
| TFF1 | 5140.13029 | 2316.89615 | 5155.55533 | 2267.56069 | 339.756248 | 208.761694 |
| NCEH1 | 2652.21769 | 1359.35912 | 2653.56188 | 1350.72376 | 24.4531202 | 32.1298549 |
| PAQR5 | 2525.69475 | 1188.68551 | 2584.42322 | 1188.95256 | 143.166172 | 26.5783595 |
| FURIN | 12598.8659 | 7665.40152 | 12446.7655 | 7552.63602 | 406.43726 | 242.62416 |
| ARRDC4 | 1307.02469 | 2971.07421 | 1245.74725 | 2973.69662 | 121.693792 | 145.493013 |
| SPINK1 | 24.4389005 | 381.678539 | 24.2786762 | 404.767762 | 7.80195856 | 40.096347 |
| CEMIP2 | 4051.53215 | 2270.76738 | 4046.87063 | 2282.35114 | 78.6389145 | 56.3183178 |
| GRN | 3192.16559 | 5468.94698 | 3157.30399 | 5392.96741 | 72.1352418 | 135.550988 |
| LPCAT1 | 4075.20721 | 7378.06744 | 4043.31058 | 7156.72843 | 120.632682 | 424.598215 |
| CDC42EP3 | 3287.87622 | 5646.71308 | 3283.48166 | 5683.2698 | 114.706799 | 130.788273 |
| AC018629.1 | 1304.61374 | 502.281382 | 1320.65133 | 520.824353 | 53.9459579 | 33.9254011 |
| SORL1 | 579.876441 | 1397.48458 | 597.357902 | 1398.07143 | 32.3889292 | 14.3812912 |
| OSMR | 6115.19344 | 3747.5168 | 6106.20139 | 3793.25217 | 28.5006347 | 89.4418662 |
| CEACAM6 | 61040.4957 | 40454.1645 | 61598.0758 | 40665.9432 | 1612.22711 | 981.5095 |
| HLA-A | 4250.50988 | 7095.72082 | 4211.59611 | 7185.04081 | 119.286682 | 227.71658 |
| PTGS2 | 2216.82332 | 829.636086 | 2300.61387 | 816.74728 | 277.648577 | 37.3390023 |
| PAM | 3542.32135 | 6002.03189 | 3527.94281 | 5944.76281 | 133.29339 | 145.393268 |
| MUC16 | 45.8081294 | 385.212011 | 44.5789479 | 371.725496 | 7.98300833 | 27.5640792 |
| ANXA9 | 389.687695 | 1061.61531 | 394.969558 | 1043.65105 | 18.7233223 | 44.8833991 |
| TMEM9 | 1521.13861 | 2988.59548 | 1503.60104 | 2967.12055 | 55.3541307 | 152.386279 |
| ZBED2 | 2345.56662 | 1117.53289 | 2327.40413 | 1117.93106 | 139.320548 | 44.1032064 |
| SYTL2 | 257.239055 | 801.400374 | 257.856285 | 810.701477 | 9.53227755 | 33.5911336 |
| SPRED2 | 2278.52749 | 1171.96434 | 2262.94006 | 1176.76509 | 58.9404811 | 24.9976085 |
| TMC4 | 540.531802 | 1281.64745 | 540.209686 | 1263.9197 | 23.5787138 | 32.5493657 |
| ARHGAP26 | 1791.59897 | 862.647217 | 1764.82429 | 855.794697 | 59.9301096 | 12.6618494 |
| NDRG1 | 1249.23142 | 512.760714 | 1254.89738 | 506.35701 | 48.1568008 | 25.7097986 |
| PERP | 8192.19055 | 13001.5109 | 8081.20254 | 12978.1761 | 236.081541 | 492.764911 |
| CHST3 | 2009.4268 | 3548.56257 | 1965.73558 | 3522.30559 | 82.3496275 | 54.8221869 |
| MSLN | 964.304306 | 1971.06984 | 942.844749 | 1938.62398 | 43.0301805 | 58.2424135 |
| MYL9 | 71.1138258 | 416.430121 | 77.8592031 | 415.05697 | 11.8169165 | 2.71099476 |
| MUC1 | 1300.35898 | 2468.35295 | 1300.8918 | 2496.81264 | 8.31237135 | 105.443236 |
| TCEA2 | 1453.27743 | 649.531043 | 1467.60412 | 652.584759 | 44.5839712 | 33.3810784 |
| ATP2C2 | 1610.04989 | 627.840518 | 1612.64344 | 635.247885 | 59.9830836 | 79.9213053 |
| CEACAM5 | 4366.41024 | 7793.03999 | 4365.39348 | 7778.14949 | 271.984347 | 372.385497 |
| CDR2L | 1785.83038 | 3277.52707 | 1794.0813 | 3223.27308 | 58.0555203 | 146.322472 |
| SPRED1 | 1438.61323 | 513.23644 | 1400.15663 | 484.069201 | 107.700398 | 72.0672545 |
| HLA-B | 440.946121 | 1110.13989 | 441.989743 | 1124.26311 | 7.64771005 | 63.391596 |
| DUSP4 | 11862.5405 | 6798.96084 | 12154.4076 | 6714.86377 | 768.155048 | 304.183895 |
| ERBB2 | 3237.78861 | 5206.39341 | 3244.3928 | 5213.24356 | 78.505614 | 60.8042396 |
| PLAU | 1559.8005 | 709.685163 | 1542.4316 | 709.582669 | 66.6046671 | 38.619896 |
| KIFC3 | 2620.28851 | 1359.55804 | 2616.23666 | 1390.30219 | 123.714173 | 60.1044555 |
| LINC02747 | 376.881687 | 969.232588 | 382.598449 | 957.681562 | 10.5848541 | 27.735805 |
| KRT13 | 72.7233019 | 437.736016 | 68.9629386 | 445.244538 | 16.9663224 | 33.4793423 |
| TMCO3 | 2024.64602 | 3510.66654 | 2000.48029 | 3457.04711 | 46.9505047 | 108.239296 |
| AJUBA | 2602.06302 | 4486.28318 | 2622.35661 | 4511.08689 | 59.5404561 | 206.765402 |
| TAGLN | 80.6759468 | 451.117768 | 85.8144748 | 458.461445 | 9.7004148 | 54.0258663 |
| ADAM8 | 1212.36621 | 459.445427 | 1198.49228 | 446.896652 | 89.8955176 | 44.6115546 |
| ABCC2 | 15340.6709 | 21999.1825 | 15299.752 | 21896.3351 | 147.317685 | 429.940127 |
| COL4A4 | 2004.15183 | 3430.01263 | 1990.45003 | 3428.13513 | 41.042401 | 77.1753313 |
| CXCL8 | 564.573299 | 102.213723 | 619.647376 | 103.257082 | 106.342399 | 8.36088855 |
| GDF15 | 3940.79219 | 1933.18265 | 4039.46941 | 1866.88805 | 249.424395 | 184.022144 |
| IGF1R | 8845.64251 | 13718.8594 | 8938.07906 | 13614.2398 | 391.370131 | 264.972959 |
| UPP1 | 2063.79552 | 1079.73135 | 2086.29476 | 1096.88765 | 62.307894 | 31.2589586 |
| LAMC2 | 12447.8051 | 8542.34721 | 12394.062 | 8544.93894 | 175.845059 | 139.170108 |
| PDCD4 | 1164.48198 | 2188.43501 | 1148.33742 | 2215.48396 | 34.3111424 | 68.1463264 |
| GABARAPL1 | 2581.96177 | 4204.67105 | 2591.12079 | 4208.68164 | 29.6611551 | 101.149072 |
| IGFN1 | 10.0385031 | 289.157611 | 8.35914408 | 288.03165 | 4.43649865 | 29.9791969 |
| DKK1 | 1146.03176 | 478.780161 | 1135.23741 | 475.808635 | 69.5508678 | 13.6107995 |
| NEBL | 1187.12264 | 2261.79166 | 1177.59442 | 2299.74173 | 28.5652745 | 118.149905 |
| STEAP4 | 1766.62242 | 3148.56702 | 1815.47765 | 3133.2329 | 100.594311 | 45.123241 |
| SORT1 | 2197.80218 | 3673.64856 | 2169.19789 | 3677.33558 | 71.1913589 | 47.1766149 |
| STAMBPL1 | 803.629398 | 294.756775 | 805.612511 | 299.858567 | 9.96263121 | 15.3457382 |
| GSN | 1103.72531 | 2055.21617 | 1092.95809 | 2053.09918 | 22.3102327 | 34.962127 |
| PDZK1IP1 | 131.150708 | 526.261675 | 129.278949 | 523.454779 | 20.3477061 | 11.1910135 |
| TMEM154 | 1102.37559 | 453.118595 | 1097.13766 | 448.487637 | 68.6961757 | 15.9312857 |
| GPAT3 | 2834.79041 | 1641.00277 | 2806.28009 | 1658.37908 | 55.3051094 | 36.2833531 |
| GSTP1 | 46388.5111 | 67631.7924 | 46631.8085 | 67844.0333 | 638.618427 | 2690.62981 |
| GCLC | 6227.24973 | 9147.01007 | 6179.49726 | 9191.53243 | 114.663746 | 97.5747818 |
| PDLIM1 | 3647.00629 | 5735.93759 | 3640.96424 | 5778.26632 | 89.1855717 | 160.577885 |
| L1CAM | 232.804077 | 696.883028 | 241.112371 | 704.954174 | 18.367862 | 29.9530488 |
| DLX5 | 308.8708 | 836.647427 | 314.512796 | 831.839054 | 26.7666491 | 24.0907474 |
| SPTLC2 | 1837.98943 | 3094.12172 | 1850.02634 | 3078.91366 | 29.3540449 | 72.9487227 |
| PTPN12 | 6952.36515 | 4531.79246 | 6933.14088 | 4495.40033 | 87.1415675 | 153.863416 |
| SLC7A5 | 40136.0152 | 61235.2829 | 39837.9589 | 61418.1385 | 1254.82327 | 3015.08187 |
| HLA-C | 7429.15683 | 11502.6928 | 7417.93694 | 11273.1952 | 127.156865 | 560.992165 |
| TNS3 | 4588.71299 | 6928.55625 | 4594.53018 | 6969.31375 | 24.7810381 | 174.73034 |
| CPS1 | 28363.6717 | 40464.2105 | 28234.4261 | 40951.0552 | 292.759539 | 1346.3652 |
| TAPBP | 1742.0452 | 3007.87377 | 1747.49476 | 2983.71665 | 25.0986959 | 126.464408 |
| CYP4F3 | 3434.76422 | 5381.75435 | 3404.71715 | 5455.50357 | 81.0186543 | 144.301703 |
| MLX | 2966.85469 | 1743.91154 | 2971.67572 | 1744.34856 | 98.1066984 | 20.7674362 |
| DAG1 | 7376.51337 | 10607.5456 | 7404.56325 | 10640.0733 | 102.361312 | 135.545202 |
| CLU | 930.281842 | 1794.67296 | 913.380543 | 1834.67184 | 32.1551895 | 71.6142285 |
| RARG | 1326.61396 | 2362.43382 | 1325.28084 | 2341.51293 | 3.59168779 | 92.2955075 |
| ITGAV | 2554.63559 | 4182.88027 | 2556.79576 | 4104.67553 | 85.5386739 | 143.40956 |
| SDC4 | 6333.08159 | 4085.88912 | 6326.68814 | 4120.37061 | 209.099031 | 103.656201 |
| TJP1 | 13443.315 | 18954.9397 | 13388.1725 | 18977.9947 | 338.668939 | 139.928369 |
| HSPB8 | 1883.79572 | 3245.47908 | 1893.49081 | 3273.56519 | 61.411014 | 145.059207 |
| LDB2 | 237.815785 | 16.3314261 | 235.252001 | 18.1732465 | 6.47886306 | 4.60288834 |
| IER3 | 754.004851 | 240.736605 | 785.289596 | 244.62962 | 74.71627 | 28.2282604 |
| CHP1 | 8993.02914 | 12976.9686 | 8975.97117 | 12919.3374 | 165.558334 | 331.376471 |
| CYFIP2 | 698.760482 | 1433.18822 | 716.796605 | 1423.5807 | 37.8091404 | 60.2262459 |
| DNMBP | 2430.51637 | 1427.17247 | 2436.23958 | 1430.97592 | 39.6486506 | 20.4158334 |
| APOL6 | 354.985372 | 870.607405 | 337.500442 | 873.30144 | 30.3805872 | 10.7462769 |
| ANTXR2 | 713.507995 | 265.904811 | 731.094746 | 265.673028 | 32.463408 | 14.0748536 |
| GJB2 | 1899.04757 | 1019.34656 | 1934.75934 | 1003.65884 | 63.7657957 | 48.0041657 |
| ANK3 | 816.072503 | 1579.69984 | 831.335362 | 1581.65362 | 34.3019329 | 54.4173773 |
| STEAP1 | 2058.16847 | 1161.66991 | 2061.77634 | 1145.55053 | 11.9255082 | 38.1340685 |
| CYP26B1 | 693.545655 | 251.969738 | 698.22124 | 240.683981 | 21.7887214 | 21.998711 |
| SPRY2 | 566.928297 | 166.708308 | 560.92114 | 165.468146 | 64.4140843 | 12.9005789 |
| PLEKHG2 | 1046.3152 | 473.148492 | 1019.81558 | 466.900619 | 47.4534394 | 10.8884522 |
| PLEK2 | 1880.38888 | 1054.67971 | 1885.6895 | 1056.59269 | 17.2735857 | 16.1055861 |
| TNC | 1397.18614 | 719.962739 | 1387.23333 | 713.712952 | 17.9606605 | 19.460336 |
| LRP1 | 564.407261 | 1199.16805 | 563.923691 | 1182.91313 | 33.0324107 | 45.6944991 |
| CXCL16 | 1740.64093 | 2939.28838 | 1739.69344 | 2970.49974 | 46.3054834 | 126.585366 |
| TFPI2 | 9974.10126 | 6390.06454 | 9759.19064 | 6370.89183 | 479.973033 | 274.118069 |
| NTN4 | 1164.57397 | 2073.99329 | 1179.11317 | 2065.9677 | 48.2548052 | 53.0958705 |
| SAMD11 | 130.843528 | 492.210219 | 140.648883 | 502.411371 | 25.9523166 | 22.219432 |
| AHNAK2 | 1391.40985 | 2663.95643 | 1356.31449 | 2651.64187 | 152.04668 | 31.8567512 |
| PFKFB3 | 2362.36222 | 3892.52184 | 2383.40096 | 3836.27267 | 51.5235684 | 209.593936 |
| TMCC3 | 672.367941 | 1404.44106 | 697.384045 | 1437.52782 | 48.4827021 | 78.7915148 |
| FECH | 5206.9478 | 7468.72739 | 5188.10195 | 7471.02547 | 98.7972808 | 31.4270632 |
| B3GNT3 | 2262.37072 | 1326.32143 | 2277.98424 | 1323.10429 | 65.8709883 | 16.3905667 |
| SEMA3A | 2921.88703 | 1663.34555 | 2861.53501 | 1619.89711 | 114.251031 | 112.008269 |
| ATP9A | 2008.4264 | 3215.95059 | 2008.43256 | 3213.06539 | 29.1180409 | 90.1722907 |
| DSTN | 9669.9105 | 13548.0551 | 9574.17039 | 13655.8567 | 230.922096 | 210.674814 |
| NR3C1 | 6871.05755 | 9871.75912 | 6895.24897 | 9868.89889 | 156.940265 | 272.145528 |
| AKR1C3 | 15014.0507 | 21820.8469 | 14621.8949 | 21638.4266 | 759.54433 | 316.430872 |
| PDXK | 6678.21614 | 9807.80432 | 6653.87869 | 9979.59048 | 178.252534 | 367.026313 |
| FLG | 77.6772488 | 344.275291 | 82.4710537 | 347.216235 | 13.1625291 | 9.44519231 |
| MYO5B | 1473.21214 | 2420.43237 | 1465.53291 | 2426.56801 | 14.7419566 | 51.16938 |
| ABHD4 | 3984.4251 | 6233.24562 | 3940.68031 | 6082.86018 | 172.267333 | 293.339306 |
| LDHA | 55896.5325 | 42370.6124 | 56105.9494 | 42046.0448 | 721.946181 | 569.01557 |
| MAB21L4 | 274.586007 | 687.938882 | 263.16655 | 677.334701 | 22.9103229 | 19.0590021 |
| ITPRIP | 1669.75695 | 922.6752 | 1632.12288 | 916.083425 | 68.5689307 | 29.4324821 |
| PSME1 | 3469.68656 | 5091.78031 | 3483.67329 | 5074.04835 | 48.3724425 | 101.376248 |
| HSPG2 | 9800.6711 | 14339.4444 | 9853.06197 | 14118.9604 | 460.402679 | 394.442364 |
| MCTP1 | 399.008852 | 113.348538 | 392.879772 | 114.625978 | 20.9942186 | 7.06176045 |
| TP53 | 1012.89171 | 1775.77016 | 1019.81558 | 1791.71689 | 29.3127779 | 44.1517504 |
| LINC01133 | 40.7892261 | 260.687449 | 44.5789479 | 243.314407 | 7.80455159 | 31.0666937 |
| AC026785.3 | 791.117588 | 1462.33266 | 797.010337 | 1471.20691 | 41.7866298 | 24.248276 |
| IVL | 360.04251 | 897.84109 | 357.353409 | 889.66302 | 10.5871539 | 113.635245 |
| SLC22A5 | 2223.01511 | 3496.92929 | 2223.37503 | 3538.00066 | 6.62441706 | 165.914296 |
| TTYH3 | 506.869793 | 1102.67921 | 499.458859 | 1091.72 | 21.2015937 | 98.3505354 |
| MYO5C | 4765.68549 | 6728.46157 | 4730.94085 | 6753.61882 | 69.015784 | 86.9879755 |
| HEG1 | 404.666631 | 895.97946 | 409.388713 | 896.670961 | 33.664107 | 32.859431 |
| FAM83A | 5427.54567 | 3693.00337 | 5445.12104 | 3745.72666 | 136.62938 | 95.3050417 |
| FTL | 107125.58 | 150434.554 | 106891.401 | 149868.523 | 3461.48254 | 6034.23351 |
| EMP2 | 994.552626 | 1727.47889 | 998.568434 | 1740.50138 | 35.7993288 | 35.3941768 |
| GAA | 4339.97689 | 6304.36535 | 4352.58061 | 6233.42354 | 134.69302 | 143.512198 |
| AKAP13 | 3669.5242 | 5369.45933 | 3688.90794 | 5376.80278 | 123.823539 | 63.7298108 |
| CPE | 2060.76319 | 3246.2625 | 2027.22766 | 3278.82604 | 74.1279038 | 102.964813 |
| RHOB | 2665.23116 | 4234.20773 | 2695.77026 | 4138.54385 | 70.085871 | 262.296663 |
| GPX2 | 9133.63665 | 13221.514 | 8984.88696 | 13168.1215 | 368.480545 | 501.98477 |
| HR | 299.449674 | 765.668273 | 299.884294 | 761.708115 | 44.0194958 | 40.0395889 |
| TSPAN1 | 4957.85667 | 7527.96245 | 4861.33427 | 7393.37561 | 243.654572 | 335.156489 |
| LIPG | 375.18951 | 110.166549 | 386.610414 | 110.003963 | 19.932476 | 2.86394013 |
| BAG1 | 3594.66693 | 5338.76724 | 3647.7215 | 5382.58518 | 111.986163 | 182.09949 |
| KIAA0040 | 845.39258 | 376.778525 | 855.767375 | 375.855779 | 69.571993 | 2.96633007 |
| SMIM14 | 1856.22851 | 2927.33647 | 1840.15622 | 2896.09905 | 67.4554591 | 84.3012772 |
| BMP1 | 1327.72918 | 2139.31311 | 1326.11804 | 2138.14426 | 8.00593732 | 7.91034267 |
| ATP2A3 | 143.752947 | 433.027426 | 140.648883 | 434.469434 | 9.7183756 | 8.89592353 |
| ROCK2 | 3579.80372 | 5326.80589 | 3526.19478 | 5267.42811 | 129.157871 | 178.096371 |
| AP1S1 | 9480.83395 | 6382.9626 | 9506.46065 | 6564.22814 | 138.910377 | 395.680964 |
| CELSR1 | 2904.3391 | 4393.56509 | 2864.88379 | 4353.31858 | 72.8391109 | 191.441589 |
| FAM13B | 875.18936 | 1569.14862 | 886.00659 | 1538.79922 | 36.3253458 | 73.3780935 |
| UBALD2 | 651.336076 | 272.096105 | 647.509219 | 257.729677 | 7.26117643 | 26.3429105 |
| GLCCI1 | 696.358062 | 1389.08277 | 704.08161 | 1312.58259 | 16.3290857 | 140.426053 |
| CTNNAL1 | 5308.66013 | 3693.13481 | 5336.28559 | 3674.70516 | 130.361468 | 64.7421693 |
| ULK1 | 632.53764 | 1214.88961 | 633.757169 | 1206.34598 | 30.0880435 | 62.2969975 |
| TMEM59 | 2783.20668 | 4185.58468 | 2728.23161 | 4223.59754 | 114.762825 | 92.8213374 |
| CASC19 | 195.350729 | 23.5775521 | 195.903801 | 23.1295864 | 12.2273005 | 6.7149818 |
| ERAP2 | 1039.61593 | 1745.73425 | 1039.80396 | 1722.92905 | 2.24232986 | 50.4039698 |
| HTRA3 | 1549.68215 | 2578.30588 | 1564.72107 | 2658.25032 | 35.9762226 | 169.962902 |
| ETS2 | 3033.20317 | 4452.57483 | 3044.88088 | 4494.57427 | 46.3562745 | 146.02306 |
| HMGA1 | 17738.2036 | 13086.5761 | 17716.7884 | 13155.7783 | 334.194413 | 394.085661 |
| NECTIN4 | 1682.1422 | 2623.83186 | 1676.00839 | 2586.47974 | 37.2828784 | 68.3888039 |
| PPARG | 469.767969 | 165.026453 | 475.880269 | 157.776822 | 25.2824284 | 14.84875 |
| LYAR | 2791.02479 | 1811.23808 | 2801.78688 | 1792.54295 | 29.6728451 | 72.5210406 |
| RAPGEFL1 | 310.32568 | 697.94 | 309.823688 | 712.845452 | 7.57019586 | 34.7953183 |
| TGM1 | 38.4942725 | 227.425833 | 38.5110037 | 227.531851 | 10.6240744 | 13.8360241 |
| SH2D3A | 2973.443 | 1969.30963 | 2978.98997 | 1951.97188 | 37.6822001 | 47.0741949 |
| MGLL | 3717.6382 | 5487.64101 | 3695.59478 | 5385.06335 | 45.4373308 | 285.836199 |
| SLC29A3 | 271.556659 | 642.651442 | 266.447717 | 654.236873 | 12.9867408 | 39.1529336 |
| DDC | 208.925002 | 535.821596 | 216.207897 | 537.762884 | 19.3069726 | 7.2154043 |
| ASS1 | 1274.51576 | 2128.47311 | 1263.81317 | 2087.30209 | 57.0552286 | 104.887764 |
| TSPAN15 | 1358.05134 | 2154.4703 | 1366.30343 | 2158.26455 | 24.3175284 | 43.2105339 |
| EPHX1 | 5486.39674 | 8066.42424 | 5484.32507 | 7960.03468 | 205.506237 | 405.966581 |
| GALNT7 | 2359.05934 | 3653.13685 | 2322.79716 | 3583.43378 | 97.6696821 | 176.492004 |
| TGFA | 3119.33438 | 2070.41714 | 3157.9023 | 2043.85515 | 82.1790334 | 47.6118096 |
| KIAA0319 | 906.841058 | 1636.31054 | 901.742667 | 1616.39679 | 64.8103363 | 88.4961243 |
| MVP | 2400.91708 | 3554.81771 | 2405.03424 | 3533.04432 | 50.0661407 | 87.3855981 |
| DTX4 | 470.518843 | 941.090317 | 454.528459 | 952.135144 | 30.4796742 | 22.331801 |
| LAMB3 | 26094.8863 | 34364.1598 | 26119.6696 | 34927.3276 | 91.2512686 | 1033.60062 |
| AKR1B1 | 6824.21344 | 9569.52709 | 6806.09088 | 9547.13125 | 115.682249 | 393.561282 |
| IDH1 | 6531.62145 | 9242.3318 | 6549.76193 | 9437.69731 | 189.063622 | 372.096934 |
| DENND3 | 636.360459 | 1197.17962 | 652.013238 | 1180.43496 | 33.9957426 | 60.1421884 |
| FSCN1 | 16469.5064 | 10995.8231 | 16508.6626 | 10536.3527 | 194.373856 | 911.876276 |
| MT2A | 1521.44467 | 847.926349 | 1515.09486 | 848.360187 | 108.224195 | 11.1310101 |
| PTPRF | 9461.40124 | 12881.4454 | 9437.36328 | 13033.7609 | 186.734778 | 393.886424 |
| BAMBI | 1054.66976 | 1771.22738 | 1038.62365 | 1785.02229 | 36.7613063 | 66.2179818 |
| RNF213 | 7326.63446 | 11084.7208 | 7071.79235 | 10886.6947 | 545.284735 | 424.965846 |
| TNFAIP2 | 3338.13915 | 2228.69187 | 3357.99208 | 2273.10711 | 62.2103831 | 82.3191907 |
| MAN2B2 | 2217.20386 | 3405.06717 | 2223.37503 | 3338.94382 | 94.1821144 | 131.347442 |
| TXN | 27708.4786 | 37432.4857 | 27985.549 | 38026.7538 | 511.596455 | 1473.18576 |
| SLC45A3 | 640.919322 | 277.652737 | 635.29495 | 286.716437 | 32.2553397 | 18.0170126 |
| LGALS9B | 241.442543 | 52.381457 | 244.461154 | 52.6085205 | 15.5032352 | 1.43925419 |
| LBH | 195.537414 | 495.016399 | 196.740997 | 493.981881 | 8.87990201 | 3.75251103 |
| IFI27 | 43.4136007 | 253.101588 | 40.1210531 | 230.469807 | 12.7326319 | 45.1841808 |
| ACSL3 | 6741.83154 | 4702.59108 | 6772.07627 | 4580.88692 | 127.788288 | 224.153634 |
| FERMT1 | 6776.66719 | 4899.27929 | 6698.40305 | 4913.63581 | 175.53412 | 67.8656955 |
| SCARB2 | 3920.93229 | 5444.50988 | 3912.07943 | 5493.72734 | 43.9559035 | 88.452999 |
| OSBPL7 | 894.883092 | 1598.00215 | 874.575449 | 1578.25561 | 72.4627068 | 58.364241 |
| APOL2 | 942.656411 | 1571.8914 | 938.386854 | 1575.29005 | 10.298608 | 35.8041944 |
| ANXA4 | 1670.70793 | 2559.06901 | 1678.39739 | 2576.31131 | 37.4272997 | 68.9617376 |
| GLP2R | 271.687626 | 625.76726 | 271.931582 | 607.628412 | 1.1706495 | 43.8147538 |
| SLC7A2 | 9135.87302 | 6263.03378 | 9096.13163 | 6284.63905 | 464.555437 | 276.536931 |
| MUC13 | 6572.01587 | 9299.51591 | 6534.15929 | 9224.90407 | 332.609856 | 195.305764 |
| LFNG | 237.472501 | 581.252477 | 236.14582 | 548.50162 | 6.42881295 | 57.012705 |
| LTBP2 | 643.529096 | 292.432302 | 647.989496 | 293.292502 | 30.8104167 | 2.97708329 |
| CTSS | 24.694424 | 173.951858 | 24.2786762 | 171.014565 | 2.9813653 | 6.58966462 |
| ARHGEF2 | 2396.56954 | 1509.71749 | 2391.86821 | 1519.07103 | 11.3579074 | 92.906661 |
| RASSF2 | 113.789109 | 363.760011 | 118.881794 | 353.121966 | 12.8360878 | 33.6694518 |
| CCDC80 | 253.975943 | 605.620405 | 245.184214 | 607.628412 | 25.936907 | 38.9206027 |
| ATOH8 | 33.4546595 | 194.771596 | 33.4365763 | 200.731768 | 3.37294753 | 14.3427048 |
| FST | 176.717965 | 27.069116 | 176.586919 | 25.6077564 | 2.64920864 | 3.97231508 |
| NUCB1 | 1342.17278 | 2145.6907 | 1339.55284 | 2110.41217 | 29.6053276 | 109.375361 |
| TACSTD2 | 9555.90189 | 13414.6915 | 9517.24108 | 13429.2031 | 83.1487594 | 765.402818 |
| IL1A | 869.263441 | 403.399764 | 918.403718 | 406.737343 | 98.8639343 | 7.72796742 |
| WDR66 | 160.363067 | 440.204671 | 156.555602 | 451.026935 | 18.7912529 | 23.532307 |
| INSL4 | 703.674055 | 1227.47098 | 708.267589 | 1222.98524 | 8.1037933 | 40.9459891 |
| TAPBPL | 106.494826 | 343.777173 | 110.332896 | 331.24872 | 9.07616732 | 29.4930107 |
| PLAAT3 | 439.97761 | 847.540019 | 439.899957 | 844.90439 | 5.37211439 | 11.300072 |
| GPR153 | 1489.02346 | 881.698248 | 1517.91318 | 886.45357 | 65.7819754 | 18.247726 |
| RASGRP1 | 220.495555 | 541.350597 | 218.382639 | 518.193927 | 15.4828886 | 40.3307849 |
| PPP1R13L | 1773.76269 | 2709.41669 | 1775.27322 | 2703.68344 | 9.13021963 | 128.672619 |
| CTSD | 6110.25538 | 8371.29619 | 6058.27902 | 8363.07441 | 157.658732 | 238.029461 |
| ABCC3 | 4761.51186 | 6751.22404 | 4683.21047 | 6814.6993 | 169.675671 | 255.927014 |
| LINC00973 | 203.033941 | 35.6723161 | 192.260314 | 35.127316 | 19.132204 | 7.02600106 |
| RHOBTB3 | 6759.51682 | 9619.19082 | 6687.31526 | 9494.69522 | 223.353028 | 511.430308 |
| EPB41L4A | 27.5539606 | 181.292779 | 27.1672183 | 194.949371 | 3.24623463 | 23.7402827 |
| HSPB1 | 2186.72514 | 3432.40265 | 2158.73555 | 3419.87456 | 80.2031178 | 244.170912 |
| DDR1 | 3847.31646 | 5374.40104 | 3851.6211 | 5293.13188 | 59.6333975 | 173.731799 |
| GNG11 | 2294.53301 | 1482.94874 | 2323.67766 | 1479.61464 | 93.3342192 | 17.7752523 |
| CCNB1 | 10967.4105 | 8433.20174 | 11029.9462 | 8414.73285 | 110.590936 | 42.1170741 |
| DGCR2 | 2279.33063 | 3288.96774 | 2283.55661 | 3310.83508 | 15.4636015 | 75.5409842 |
| YRDC | 5658.09764 | 3872.96718 | 5648.5596 | 3912.20433 | 264.033713 | 178.41465 |
| STOM | 2932.25882 | 4176.9659 | 2981.254 | 4159.81373 | 107.791415 | 45.3684918 |
| 4-Mar | 143.724739 | 13.5271519 | 148.225002 | 12.3908499 | 10.7651314 | 3.15961871 |
| PHC2 | 2424.62343 | 1608.11922 | 2416.17898 | 1573.33399 | 19.686229 | 60.7025151 |
| PSMB8 | 416.037593 | 820.179721 | 420.156584 | 836.795394 | 15.8787943 | 41.6365018 |
| SLC3A2 | 18253.1163 | 23617.2841 | 18321.1914 | 23635.693 | 193.428713 | 613.760785 |
| LUCAT1 | 1673.22523 | 1028.28725 | 1671.71055 | 1033.39688 | 48.146921 | 42.1858215 |
| PHGDH | 1675.04623 | 2622.41288 | 1688.42765 | 2564.66537 | 74.7525311 | 136.251939 |
| CAPN2 | 22388.2395 | 17664.0915 | 22395.1919 | 17753.1606 | 108.825997 | 229.980076 |
| PLXNB1 | 1071.83161 | 1708.0602 | 1077.28469 | 1706.63305 | 32.9843436 | 31.9011294 |
| C6orf132 | 1656.03488 | 2459.13653 | 1661.83353 | 2471.28525 | 24.6640034 | 38.1519677 |
| TBC1D2 | 3064.17218 | 4404.08633 | 3037.34611 | 4498.70456 | 100.278609 | 166.723126 |
| LGALS3 | 4895.99429 | 6578.24501 | 4907.64138 | 6639.19528 | 20.3651264 | 136.556054 |
| RBMS2 | 4289.65958 | 3087.4313 | 4251.27993 | 3103.90271 | 74.9369861 | 37.0666636 |
| SLCO4A1 | 2586.69754 | 1725.40595 | 2587.80793 | 1697.54643 | 50.1962241 | 75.6624906 |
| FRMD6 | 934.793319 | 503.553382 | 928.909886 | 520.824353 | 12.8498956 | 31.1900998 |
| TIMP2 | 2316.81393 | 3291.13201 | 2307.12377 | 3301.74846 | 30.3691048 | 35.0029257 |
| S100A16 | 19635.474 | 14957.3617 | 19746.245 | 14755.3748 | 561.88138 | 356.321698 |
| JCAD | 302.370556 | 89.5035489 | 303.136846 | 80.2279937 | 11.4996564 | 16.1528447 |
| ANKRD2 | 157.186225 | 413.786905 | 161.598686 | 425.419179 | 11.4417394 | 25.4938529 |
| F3 | 9843.18944 | 7334.32331 | 9971.00116 | 7312.02814 | 351.342128 | 105.686744 |
| COBL | 3057.90179 | 4348.39888 | 3071.67114 | 4421.05523 | 75.2092263 | 175.552756 |
| CREG2 | 1010.29973 | 1704.14231 | 1001.0075 | 1658.72177 | 47.8294248 | 124.75481 |
| SAMD9L | 454.843363 | 867.395645 | 455.819743 | 860.751037 | 2.09474602 | 59.5103873 |
| FA2H | 2314.56065 | 3300.20241 | 2305.63705 | 3344.58669 | 21.6818586 | 84.2349686 |
| IRS1 | 1366.02477 | 773.777442 | 1301.00217 | 766.33013 | 120.031993 | 21.8086924 |
| MB | 22.7905807 | 154.042759 | 24.0325392 | 151.602101 | 2.36749708 | 6.97212836 |
| SCD | 40966.9003 | 31735.0696 | 41350.3176 | 32078.2582 | 1213.77913 | 640.927268 |
| CRYBG2 | 1693.01155 | 2488.31671 | 1681.92623 | 2502.85036 | 40.2640985 | 30.4110887 |
| CA2 | 617.450931 | 277.076219 | 645.477909 | 269.001288 | 55.2600659 | 16.9504345 |
| ELF3 | 1992.22712 | 3035.13161 | 2018.31187 | 3008.93193 | 139.918897 | 66.6771794 |
| ID3 | 2418.9734 | 3537.24296 | 2436.23958 | 3613.99788 | 93.9649997 | 144.667468 |
| IGFBP2 | 1194.54108 | 2008.91 | 1186.99846 | 1938.75497 | 13.6679708 | 200.010446 |
| CADM1 | 271.377128 | 587.107654 | 265.402824 | 585.26979 | 20.5197665 | 23.0588289 |
| TOR4A | 2071.97801 | 1285.18986 | 2056.20397 | 1223.38991 | 28.0943593 | 119.612474 |
| UCHL1 | 20596.789 | 26847.3861 | 20422.7305 | 27292.912 | 319.087262 | 968.492899 |
| TOP1 | 6212.1037 | 4585.45977 | 6264.45666 | 4562.85347 | 174.385408 | 99.1837014 |
| NADSYN1 | 1824.65951 | 2683.44282 | 1856.90013 | 2691.86169 | 66.5714968 | 63.6933826 |
| SPHK1 | 918.360943 | 510.145888 | 912.753959 | 500.590334 | 17.9993406 | 21.984953 |
| KIRREL1 | 2174.6283 | 1459.34366 | 2176.51214 | 1444.10389 | 29.6729996 | 28.1769066 |
| OPTN | 1095.81732 | 1763.01857 | 1102.58678 | 1710.14565 | 22.2110255 | 111.643328 |
| MEGF9 | 1368.14024 | 2130.17021 | 1401.20153 | 2145.53948 | 84.9647189 | 53.2517863 |
| LINC00963 | 691.672109 | 1167.50538 | 692.764065 | 1162.89904 | 21.3483926 | 26.027605 |
| OLMALINC | 547.546488 | 235.655611 | 571.804685 | 243.118003 | 51.8948071 | 18.6971061 |
| PITPNC1 | 509.69467 | 221.21801 | 516.549766 | 226.478748 | 14.3620486 | 23.0751328 |
| ARHGAP23 | 567.643297 | 1019.65543 | 587.229871 | 1030.09265 | 38.0387823 | 37.5233319 |
| ECT2 | 6103.77458 | 4323.77274 | 6109.01725 | 4230.23614 | 142.200913 | 252.787391 |
| NTNG1 | 629.627486 | 309.238072 | 618.532903 | 304.128604 | 28.7029622 | 8.97116076 |
| COL8A1 | 314.963459 | 109.743816 | 318.739478 | 107.847467 | 7.42115036 | 6.01147374 |
| SH3BGRL2 | 470.550189 | 879.96877 | 478.038763 | 871.489774 | 27.665055 | 50.8957146 |
| SERINC5 | 1358.32465 | 2023.0418 | 1357.42896 | 2013.92613 | 1.84880274 | 20.4564591 |
| BCL2L1 | 4510.69933 | 3198.57796 | 4548.16716 | 3170.97857 | 116.388518 | 142.60367 |
| PRKCA | 1471.50131 | 892.671682 | 1472.25425 | 898.519767 | 54.5935518 | 52.2032065 |
| FN1 | 254.354143 | 545.063633 | 247.809937 | 555.019891 | 13.3401329 | 17.3229043 |
| LETM2 | 299.860408 | 100.713007 | 296.450004 | 102.586615 | 16.3508266 | 5.83585261 |
| MTURN | 534.487276 | 952.277672 | 531.850542 | 968.774399 | 14.4139775 | 45.4140981 |
| GLUL | 4650.16625 | 6298.70532 | 4607.97817 | 6381.15427 | 135.89786 | 175.349533 |
| PALLD | 5097.73703 | 6829.2545 | 5145.05318 | 6856.27026 | 181.994208 | 48.4263936 |
| C1orf116 | 4690.81045 | 6271.99489 | 4699.92876 | 6254.51105 | 126.293258 | 101.684514 |
| SPTAN1 | 12832.8259 | 16795.6842 | 12703.8857 | 16811.9051 | 519.772927 | 142.54196 |
| TPRA1 | 1246.42504 | 1907.77126 | 1214.16568 | 1900.75637 | 57.5336146 | 28.019761 |
| NAV3 | 949.739428 | 533.362137 | 958.447381 | 535.284714 | 34.5132804 | 19.5388444 |
| MXD4 | 605.984275 | 1099.86651 | 607.804101 | 1087.09799 | 50.2608886 | 70.4232352 |
| ITM2B | 2310.61541 | 3322.85618 | 2312.33461 | 3286.25285 | 84.1468695 | 115.703373 |
| TUFT1 | 2267.40699 | 3269.7851 | 2290.40548 | 3299.86945 | 100.284422 | 81.8281675 |
| KIAA1522 | 7148.73658 | 9166.7471 | 7133.74614 | 9161.75865 | 41.5458976 | 50.5866944 |
| CDC42BPB | 4670.27392 | 6138.57481 | 4669.87779 | 6142.5573 | 7.52185249 | 81.8162916 |
| TTC9 | 1589.60605 | 2349.66292 | 1615.78776 | 2371.32906 | 49.5998411 | 69.2380524 |
| G6PC3 | 2307.535 | 3359.19105 | 2344.85266 | 3338.01062 | 88.1966561 | 158.545313 |
| SERPINE2 | 1295.669 | 790.10731 | 1319.53686 | 780.623541 | 58.7005801 | 17.0381329 |
| TSPAN8 | 2070.41333 | 2985.78615 | 2096.32503 | 3000.23778 | 76.5678135 | 97.7332857 |
| PRKCD | 1570.70709 | 2313.88345 | 1573.09078 | 2335.81831 | 60.5530522 | 42.5985846 |
| RHOU | 451.287969 | 861.180071 | 437.810171 | 875.40969 | 38.5645929 | 50.9973863 |
| LAMP2 | 1977.73153 | 2824.51215 | 1945.59078 | 2824.05132 | 74.4852245 | 9.54981881 |
| B4GALT5 | 3221.16609 | 4441.21933 | 3275.4382 | 4411.22444 | 96.6294896 | 125.336647 |
| ARHGAP21 | 6709.7134 | 8944.2047 | 6704.03355 | 8891.67386 | 171.777624 | 324.830818 |
| AFF1 | 2017.49312 | 3014.56803 | 1998.88033 | 2903.58915 | 51.2116487 | 203.363659 |
| BNIP2 | 3325.79633 | 2170.12941 | 3265.29066 | 2091.57546 | 122.817498 | 189.998987 |
| CBX6 | 2176.6182 | 3148.94485 | 2168.153 | 3238.05444 | 38.4237366 | 157.28893 |
| FKBP4 | 11165.4159 | 8634.64578 | 11122.8861 | 8773.54776 | 143.703321 | 240.791632 |
| TNFSF15 | 1527.61457 | 940.052333 | 1517.18465 | 949.139099 | 46.9977777 | 67.925745 |
| CXXC5 | 1736.41632 | 2591.00398 | 1770.89871 | 2569.84048 | 78.3388448 | 115.788911 |
| CTSA | 2602.36012 | 3613.89401 | 2623.47109 | 3582.60772 | 72.8105398 | 67.9054509 |
| SLC16A14 | 2840.08186 | 1890.34013 | 2854.83745 | 1929.66835 | 146.841218 | 102.29048 |
| ADGRE5 | 1973.63578 | 1283.15597 | 2010.51055 | 1279.56176 | 91.6297463 | 59.2764071 |
| HTT | 4381.5781 | 5954.68542 | 4356.47769 | 5993.4257 | 111.500022 | 221.364016 |
| LINC00460 | 138.28403 | 16.2447343 | 129.566733 | 12.9416427 | 20.3855157 | 6.70064849 |
| RAC2 | 607.300085 | 296.046568 | 591.897382 | 287.489349 | 27.1019291 | 21.5022296 |
| UBL3 | 1228.48355 | 1862.86342 | 1223.98016 | 1843.75846 | 20.086505 | 78.9043306 |
| CEMIP | 401.807786 | 158.075948 | 402.283809 | 154.37531 | 47.1689396 | 7.79507726 |
| FRK | 1083.45064 | 1771.15751 | 1097.75659 | 1704.51606 | 46.5514659 | 147.595202 |
| IL4R | 1165.04757 | 711.731867 | 1175.76975 | 710.865947 | 18.9975166 | 16.4342626 |
| INSYN2B | 149.044462 | 25.4098289 | 154.644165 | 24.9588824 | 10.978428 | 3.29987155 |
| RNF145 | 3251.60492 | 4521.72868 | 3273.20925 | 4536.04578 | 125.291756 | 156.446316 |
| FOSL2 | 7464.53305 | 9825.21865 | 7413.3682 | 9841.73897 | 142.193737 | 350.439012 |
| CELSR3 | 728.850273 | 1232.94113 | 720.825525 | 1191.55553 | 18.7091654 | 91.2694568 |
| DSP | 10536.9435 | 13544.1916 | 10390.2383 | 13593.3469 | 348.494029 | 94.8076269 |
| TPM3 | 25517.8275 | 20358.8131 | 25336.0544 | 20378.8177 | 351.200812 | 447.672766 |
| IGSF9 | 1418.85231 | 2086.36201 | 1391.41931 | 2091.57546 | 48.9692714 | 20.6848322 |
| MFSD2A | 390.550917 | 160.397846 | 381.385949 | 155.195135 | 20.0768196 | 11.4008128 |
| SLC41A2 | 591.776227 | 1037.0645 | 611.152884 | 1007.78912 | 43.1822877 | 52.142931 |
| CHST11 | 1610.02695 | 1055.46375 | 1605.9566 | 1060.65675 | 28.2253009 | 29.8932955 |
| FLG-AS1 | 58.7229101 | 218.771023 | 58.5140085 | 228.847064 | 10.2716018 | 18.1465392 |
| FIBP | 1532.33087 | 2254.61225 | 1530.76826 | 2251.64468 | 50.8801523 | 71.7776482 |
| VWA7 | 95.9941925 | 283.816077 | 98.0736855 | 293.960171 | 7.40573636 | 25.1752409 |
| KIF1C | 9675.48817 | 12530.371 | 9740.77234 | 12364.416 | 258.548098 | 340.443412 |
| BCCIP | 4813.94403 | 3624.34028 | 4843.0791 | 3612.34576 | 62.7923153 | 58.45017 |
| LIPA | 2644.24838 | 3692.96743 | 2703.71319 | 3649.71611 | 109.226028 | 94.3096004 |
| CDKN1C | 86.3250451 | 289.813301 | 83.5914408 | 255.251507 | 5.36054509 | 59.9634987 |
| DLC1 | 221.991552 | 55.6327499 | 210.973324 | 55.345796 | 21.3456099 | 17.8774291 |
| HIF1A | 9617.74933 | 7446.68159 | 9600.96065 | 7461.76979 | 300.825123 | 58.3292839 |
| ANKRD22 | 908.592563 | 516.804205 | 898.265801 | 508.987436 | 39.1702143 | 29.1789485 |
| DUSP5 | 449.11182 | 205.077938 | 443.034636 | 209.818391 | 12.0310495 | 9.04190476 |
| PCDH7 | 1007.99976 | 1546.94426 | 1009.65805 | 1539.76961 | 27.099055 | 47.8572901 |
| AL590004.3 | 12.9155232 | 114.197046 | 11.144737 | 118.126102 | 3.29587944 | 7.79711628 |
| ABCG2 | 1240.38884 | 1830.26667 | 1253.78291 | 1836.03736 | 25.1094657 | 23.8041057 |
| DHRS9 | 1415.15692 | 899.191655 | 1392.84238 | 888.351333 | 52.510043 | 32.6875223 |
| LINC01484 | 1122.41205 | 1744.21071 | 1114.90084 | 1741.32743 | 82.1636935 | 11.7408271 |
| CLSTN1 | 4389.38088 | 5794.04458 | 4382.71966 | 5808.00436 | 75.4344751 | 154.227054 |
| PGRMC2 | 1752.4865 | 2524.10673 | 1707.87929 | 2493.86505 | 77.7162845 | 63.1113753 |
| MRGBP | 2218.77337 | 1489.81762 | 2200.54468 | 1439.81675 | 72.998926 | 90.2822892 |
| ERV3-1 | 175.229865 | 399.042305 | 174.972371 | 401.139969 | 5.41247431 | 5.43477968 |
| TMPRSS3 | 437.211544 | 782.280926 | 440.364955 | 789.440206 | 13.7715535 | 32.2616763 |
| ACTG1 | 34214.7611 | 27853.0013 | 34498.3245 | 27833.9791 | 623.097785 | 82.1714331 |
| RDX | 18973.6547 | 23715.6982 | 18959.1345 | 23741.6944 | 279.702494 | 554.09349 |
| PTPRS | 2639.33459 | 3771.0734 | 2601.18161 | 3648.69226 | 72.9040455 | 232.337186 |
| MYO15B | 266.413548 | 541.055981 | 264.553851 | 533.632601 | 12.4937429 | 24.271249 |
| CHD3 | 3648.82767 | 4831.15878 | 3644.32899 | 4847.87516 | 85.5491495 | 52.1570017 |
| BCAR3 | 2947.52641 | 2075.07501 | 2916.78993 | 2102.09254 | 106.405741 | 76.6239496 |
| FHOD3 | 333.904979 | 128.237057 | 339.901467 | 130.206088 | 23.8834147 | 13.3659458 |
| EFNA1 | 1069.16517 | 1616.15259 | 1064.07578 | 1618.62974 | 28.900547 | 39.6819931 |
| PRDM1 | 602.39357 | 305.692134 | 622.873624 | 309.675022 | 38.5083505 | 14.9832911 |
| VSIR | 383.642381 | 713.72014 | 390.970407 | 723.367157 | 27.5455598 | 24.4605577 |
| ITGA5 | 1357.9028 | 873.904387 | 1352.97107 | 859.92498 | 34.6635574 | 26.8560379 |
| LAMP1 | 8568.91521 | 11529.3857 | 8563.94311 | 11502.839 | 64.7986564 | 715.495615 |
| SLC6A14 | 1462.07584 | 844.832828 | 1423.18291 | 827.268984 | 120.644338 | 85.1003534 |
| FKBP14 | 837.478046 | 485.575487 | 838.084221 | 483.462796 | 9.20336387 | 15.8852578 |
| ARHGEF17 | 1302.69704 | 1937.30912 | 1324.44365 | 1893.32186 | 45.1974749 | 76.3132596 |
| AF121898.1 | 145.837325 | 27.0479885 | 140.015663 | 27.7320916 | 18.0277675 | 3.08944084 |
| ERAP1 | 956.595421 | 1489.8105 | 956.077104 | 1503.28847 | 11.7894311 | 80.6765177 |
| TTC3 | 7619.10769 | 9685.79898 | 7613.09047 | 9559.9537 | 25.9312199 | 237.303799 |
| SLC44A2 | 6839.5935 | 8772.50501 | 6739.22245 | 8762.80902 | 184.515227 | 90.2047999 |
| EEF1A2 | 34611.4736 | 43509.4999 | 34822.8452 | 43126.766 | 976.80315 | 1057.80993 |
| GALNT2 | 9409.27478 | 12075.2317 | 9393.33611 | 12129.816 | 244.518294 | 328.998206 |
| PCK2 | 1003.08188 | 1548.35464 | 995.783038 | 1555.89699 | 33.6712903 | 72.3050492 |
| HSPD1 | 34677.8646 | 28079.7744 | 34578.6444 | 28056.557 | 219.847753 | 687.898702 |
| NRP2 | 2385.05048 | 1682.96519 | 2386.00784 | 1675.94273 | 37.4993605 | 42.8934885 |
| PFKP | 13981.6741 | 10848.5556 | 14007.959 | 10808.9514 | 270.942239 | 426.756928 |
| CSNK1E | 2365.11965 | 1656.19115 | 2371.77551 | 1642.70105 | 26.0565068 | 62.9634588 |
| H3F3B | 17161.1119 | 13693.9208 | 17060.3746 | 13775.5411 | 437.20173 | 158.652185 |
| STK38 | 1362.26504 | 2004.36495 | 1345.37354 | 2034.63453 | 32.5415222 | 82.0361198 |
| RBM47 | 5927.52288 | 7806.22857 | 5941.57811 | 7775.15407 | 209.464043 | 230.231242 |
| LINC00511 | 628.813525 | 1115.40174 | 608.127732 | 1098.20286 | 66.5447022 | 74.4598578 |
| NOP53 | 3922.23807 | 5730.8125 | 3929.63426 | 5634.53246 | 19.0627099 | 552.237876 |
| TMPRSS11E | 1628.07456 | 1066.47499 | 1637.34735 | 1094.52507 | 28.6598835 | 61.8075664 |
| ADAM15 | 12395.4903 | 16125.2342 | 12542.8665 | 16386.2389 | 335.946089 | 679.117876 |
| MAFF | 160.6767 | 35.3690565 | 151.568423 | 33.868323 | 16.7410424 | 4.73612707 |
| FSTL3 | 2284.9033 | 3111.53968 | 2293.9163 | 3118.36388 | 43.9790728 | 24.1295358 |
| IFIT3 | 156.135274 | 365.091565 | 154.044015 | 367.595213 | 7.96971167 | 18.0692787 |
| EPS8L2 | 5836.54394 | 7826.42497 | 5877.95123 | 7768.23681 | 82.3688081 | 434.569763 |
| ATP8B1 | 1192.66496 | 1780.99905 | 1197.1899 | 1753.17894 | 56.8288474 | 48.6288285 |
| CPEB2 | 733.54394 | 1204.6111 | 713.661926 | 1187.63735 | 60.3308643 | 40.3893166 |
| THSD4 | 1640.42193 | 1083.51554 | 1682.85528 | 1074.52903 | 75.7220686 | 16.7009935 |
| CXCL1 | 257.839553 | 77.7094688 | 268.588161 | 79.3014391 | 46.5173153 | 7.33787593 |
| MAN2B1 | 634.454388 | 1037.32038 | 629.67764 | 1025.86615 | 20.7719793 | 32.3828889 |
| TTC22 | 1343.82328 | 2015.39578 | 1331.97841 | 2015.57824 | 21.6487084 | 135.068826 |
| CMIP | 9529.7099 | 7484.04501 | 9441.65324 | 7399.81554 | 202.819516 | 166.277788 |
| NQO1 | 42229.4048 | 51134.2125 | 42059.1229 | 51221.1731 | 364.996294 | 725.936477 |
| FLNB | 27803.6916 | 22700.251 | 27966.603 | 22655.8593 | 473.754737 | 237.624254 |
| JUND | 786.886528 | 1423.75563 | 793.073794 | 1338.21178 | 39.0579921 | 216.840701 |
| ALDH3A2 | 7879.42401 | 9962.73631 | 7910.53431 | 9986.19893 | 199.318167 | 77.7717848 |
| PGD | 29130.4557 | 36444.9249 | 28892.7306 | 36714.0881 | 613.056375 | 1094.43782 |
| LRWD1 | 2230.47733 | 1567.60403 | 2204.33636 | 1588.50695 | 59.0121234 | 37.4819492 |
| FAM20C | 420.412301 | 768.929039 | 417.76067 | 740.446845 | 17.5157059 | 65.9806983 |
| CDYL2 | 535.610989 | 271.027943 | 531.603954 | 270.946584 | 31.0360106 | 10.3872009 |
| SMAD6 | 914.463268 | 1443.92955 | 919.440801 | 1388.45338 | 33.4979959 | 98.1125735 |
| GREB1L | 508.231899 | 254.935347 | 516.001322 | 249.469111 | 13.9347013 | 11.4343781 |
| PLXNB2 | 9877.97991 | 12880.2196 | 9833.48812 | 12643.6232 | 121.422444 | 657.215255 |
| NRP1 | 1646.40536 | 1108.63279 | 1646.07765 | 1124.07411 | 26.9865436 | 40.4919123 |
| BTBD11 | 1402.93864 | 2029.85412 | 1393.09212 | 2020.53458 | 59.5190489 | 22.6507651 |
| MFSD6 | 708.788871 | 1134.35231 | 694.853852 | 1123.19191 | 39.5157289 | 24.3312468 |
| ZNF185 | 5660.67635 | 4324.58209 | 5615.25503 | 4304.94501 | 134.47515 | 108.645256 |
| RAB15 | 720.2142 | 1129.81535 | 720.976177 | 1131.46934 | 4.39096552 | 4.93758597 |
| RREB1 | 3090.7365 | 2278.83506 | 3079.2059 | 2270.3339 | 36.4375682 | 53.244122 |
| SIK2 | 2458.83329 | 3312.19508 | 2450.72766 | 3319.53136 | 46.4356677 | 44.4078675 |
| C3 | 2721.92471 | 3801.49841 | 2666.93556 | 3837.79157 | 117.969005 | 175.471429 |
| HMMR | 2917.82639 | 2086.83469 | 2882.85981 | 2042.52581 | 76.6602989 | 90.3949211 |
| CD47 | 1521.93826 | 2164.01058 | 1522.37107 | 2174.19598 | 37.975637 | 53.9873247 |
| FYCO1 | 977.183571 | 1491.53323 | 952.728743 | 1489.21332 | 59.3867332 | 13.3774426 |
| MIA2 | 2355.43698 | 3194.32134 | 2350.42503 | 3248.05478 | 12.9097054 | 96.5496234 |
| STK17A | 5068.22826 | 3793.32172 | 5155.50211 | 3850.9437 | 184.999795 | 109.657692 |
| GLB1 | 1687.50077 | 2364.76559 | 1688.42765 | 2334.5031 | 26.7143976 | 63.4608815 |
| MYLK | 1028.56735 | 3576.86487 | 1030.17468 | 3631.76969 | 3.66036167 | 103.868996 |
| CCND3 | 6731.53165 | 14706.2668 | 6714.85713 | 14652.7171 | 91.3059693 | 316.776076 |
| IER3 | 4554.99363 | 1071.70688 | 4639.14636 | 1036.10402 | 245.844873 | 87.6550823 |
| CD24 | 457.549722 | 2472.50974 | 452.903513 | 2414.13465 | 26.2234835 | 154.69117 |
| ST3GAL5 | 2374.77494 | 457.218899 | 2334.49407 | 445.276053 | 117.187584 | 27.3072159 |
| ANGPTL4 | 4155.05707 | 1227.35469 | 4155.82709 | 1232.48975 | 19.5833785 | 89.4560257 |
| KRT80 | 5079.35918 | 12638.1146 | 5088.8472 | 12519.2138 | 241.670718 | 306.536 |
| ITGA2 | 5704.86365 | 2168.9794 | 5694.28333 | 2180.92285 | 26.2158949 | 75.7083647 |
| TUFT1 | 2391.83668 | 6150.35683 | 2373.33812 | 6111.95621 | 79.877621 | 77.1731368 |
| FSTL3 | 3105.44008 | 7169.23741 | 3118.65299 | 7180.49431 | 72.2891192 | 21.0555827 |
| CGN | 1336.29937 | 3884.65254 | 1327.80203 | 3876.92517 | 45.6083369 | 22.5522653 |
| CXCL8 | 1760.83074 | 306.088077 | 1753.71718 | 297.070071 | 12.8406988 | 23.6806313 |
| MUC1 | 2443.19162 | 6365.60592 | 2453.98553 | 6517.97359 | 85.7582108 | 264.887162 |
| HMGA1 | 28000.8639 | 13677.607 | 27640.9403 | 13626.8936 | 859.113316 | 377.297807 |
| CAVIN1 | 36844.8124 | 70809.0459 | 36175.1647 | 70166.0721 | 1220.30995 | 1171.81743 |
| CAV1 | 12290.601 | 27506.4541 | 11975.7019 | 27521.8395 | 692.080366 | 455.926098 |
| VDR | 4141.0596 | 1401.46677 | 4107.23229 | 1383.35182 | 110.360159 | 89.1336519 |
| CA2 | 3130.1841 | 997.308562 | 3157.68995 | 1003.37306 | 82.2666822 | 53.3602692 |
| TUBA1A | 5824.23394 | 12464.8091 | 5808.0502 | 12423.3052 | 141.081013 | 487.043981 |
| KRT18 | 10143.2656 | 4782.21842 | 10158.2565 | 4813.00483 | 228.029447 | 167.440749 |
| ACTN4 | 13724.7518 | 24232.7265 | 13738.8627 | 24196.0762 | 90.5992185 | 431.428153 |
| SMOX | 3372.80573 | 1170.60969 | 3437.11589 | 1163.64188 | 129.900251 | 40.2805973 |
| STAC | 1093.61899 | 3122.75213 | 1071.02934 | 3124.16423 | 54.3778908 | 60.0212858 |
| DNAJB2 | 2965.58937 | 6319.20692 | 2979.1538 | 6260.78111 | 37.2451749 | 139.548154 |
| MALSU1 | 1581.90117 | 4076.57888 | 1598.66292 | 4081.86934 | 83.8972563 | 54.8728804 |
| MRAS | 1971.51901 | 4674.99399 | 1963.38044 | 4695.54445 | 81.6677969 | 52.7472513 |
| COL12A1 | 645.960106 | 2106.49398 | 639.507536 | 2080.10861 | 15.6030537 | 56.7424346 |
| DOCK4 | 5257.79599 | 2269.47898 | 5254.27722 | 2285.26828 | 12.6613232 | 113.454879 |
| BCAM | 2321.03503 | 5982.01185 | 2368.51043 | 5880.34355 | 100.223737 | 357.69492 |
| CLU | 12565.457 | 23474.3408 | 12484.0035 | 23545.9084 | 426.172054 | 536.12654 |
| TUBA4A | 10700.3138 | 20438.987 | 10468.2913 | 20372.1966 | 441.075366 | 356.007325 |
| PRDX6 | 9837.92625 | 17340.4227 | 9750.65613 | 17400.4528 | 208.830457 | 236.598998 |
| LXN | 2457.22953 | 764.585892 | 2523.11189 | 758.45523 | 119.135924 | 41.7050246 |
| FBLIM1 | 1848.03623 | 4291.34386 | 1837.60889 | 4315.97143 | 50.2294383 | 139.907948 |
| NCEH1 | 10486.194 | 4944.2794 | 10253.5023 | 4998.52685 | 470.033977 | 167.385024 |
| KIAA1522 | 6093.37429 | 10767.9461 | 6121.20623 | 10770.8449 | 50.1557109 | 19.0830639 |
| OPTN | 5116.96734 | 9709.21789 | 5092.73478 | 9760.94697 | 108.903224 | 248.494668 |
| OLFM2 | 945.000695 | 2750.99487 | 944.682865 | 2794.59796 | 66.7739583 | 106.359421 |
| SRGN | 17014.299 | 9967.17917 | 17063.6071 | 9920.73135 | 307.253374 | 102.352608 |
| CRIP2 | 648.838701 | 2060.94163 | 656.029767 | 2049.63497 | 48.3433432 | 41.125724 |
| SUSD2 | 2527.25123 | 730.912445 | 2547.33929 | 729.110236 | 226.43417 | 12.1358573 |
| CTIF | 2410.05466 | 5136.26522 | 2381.01883 | 5142.14588 | 55.8549675 | 175.968396 |
| CDH4 | 2036.65502 | 698.338425 | 2042.10768 | 698.636589 | 45.2040774 | 12.1983434 |
| OXTR | 305.943125 | 1356.08809 | 322.329552 | 1331.69567 | 41.0566154 | 45.4791314 |
| CITED2 | 4506.37912 | 9087.26768 | 4460.03052 | 9168.05333 | 111.950007 | 430.520363 |
| HLA-B | 8530.44242 | 15781.9697 | 8392.78937 | 15756.4557 | 288.160409 | 505.339537 |
| DSTN | 6174.68044 | 11265.9381 | 6206.97045 | 11306.2756 | 85.0281017 | 393.332466 |
| PHLDB2 | 3690.33825 | 6854.56286 | 3697.0922 | 6865.10294 | 92.5758763 | 23.8804648 |
| NDST1 | 3894.97386 | 7527.36171 | 3902.16224 | 7683.87382 | 15.558973 | 304.309854 |
| SPX | 2487.48336 | 985.462054 | 2496.90831 | 973.201952 | 43.9512435 | 29.5551248 |
| DAG1 | 13293.8349 | 22656.4438 | 13210.8141 | 22834.92 | 360.964001 | 497.112111 |
| PDLIM1 | 6742.90463 | 11522.7194 | 6768.62207 | 11626.8294 | 103.17615 | 182.062243 |
| TMSB4X | 14662.655 | 26510.0994 | 14723.2518 | 26191.7366 | 214.253567 | 1258.86298 |
| ZFAND5 | 4686.10289 | 8599.54624 | 4729.39465 | 8637.34168 | 147.083735 | 160.871674 |
| SEMA4B | 8748.85534 | 4172.00309 | 8779.13613 | 4113.18759 | 218.765138 | 282.835182 |
| ABHD4 | 6311.48237 | 11054.7616 | 6325.0613 | 11124.6695 | 153.044001 | 232.265013 |
| MYH9 | 42583.5244 | 70266.9061 | 42630.2721 | 71474.5549 | 920.094632 | 2112.98211 |
| THBS3 | 1993.61928 | 4606.32596 | 2017.65599 | 4573.47007 | 115.207836 | 217.233106 |
| PSMB9 | 2259.42199 | 4677.6796 | 2304.21177 | 4675.91511 | 108.913133 | 65.1872225 |
| PSAP | 35226.4878 | 61783.4267 | 34695.4919 | 62711.1398 | 1003.13106 | 2588.14336 |
| TRIM2 | 1733.31728 | 608.927161 | 1727.7475 | 611.73026 | 33.6875771 | 12.6812316 |
| MAP1LC3A | 542.668427 | 1607.65188 | 534.542773 | 1610.58871 | 17.3697569 | 55.1371589 |
| ARID5B | 1654.41782 | 4045.19684 | 1726.98369 | 4031.2463 | 146.291976 | 96.9965364 |
| UPP1 | 2997.18682 | 1362.25057 | 2982.74867 | 1362.68936 | 29.911166 | 36.3726425 |
| PIEZO1 | 5818.99428 | 10038.491 | 5751.5202 | 10040.5026 | 167.916492 | 110.896597 |
| SAT1 | 3025.22181 | 1251.60262 | 3073.24245 | 1212.17398 | 115.62167 | 71.2746869 |
| MMP24 | 63.4009868 | 595.901848 | 64.3259119 | 597.145147 | 6.61721875 | 17.6116605 |
| CNTNAP1 | 1273.37647 | 2955.02459 | 1280.15244 | 2896.12518 | 39.5926485 | 126.653785 |
| CADM1 | 1109.72968 | 2581.07597 | 1131.20869 | 2590.03959 | 39.823666 | 51.4998691 |
| CCN1 | 26564.6399 | 44948.5855 | 26732.6969 | 44905.7414 | 1164.08086 | 440.4174 |
| RHOB | 3296.90775 | 7322.0361 | 3236.45745 | 7385.79698 | 238.732742 | 358.49346 |
| UPK3B | 52.7675449 | 562.272534 | 54.9946155 | 575.353103 | 4.46874866 | 23.6412041 |
| SEMA3C | 10485.4645 | 16964.0956 | 10539.8708 | 17165.3402 | 142.450524 | 365.082795 |
| TNS3 | 9551.29267 | 16759.8354 | 9530.79593 | 16712.2463 | 317.363562 | 550.620795 |
| TM4SF1 | 21356.1545 | 13015.2846 | 21635.3758 | 13016.3178 | 694.885268 | 21.2002294 |
| COL4A4 | 3491.19514 | 7380.34245 | 3493.96594 | 7153.16551 | 107.667629 | 510.852571 |
| JAK1 | 9801.67677 | 15900.7807 | 9772.73816 | 15923.5262 | 90.9871599 | 391.232688 |
| SLC4A7 | 2787.70051 | 1195.69751 | 2782.57478 | 1195.32543 | 95.3990313 | 50.1510771 |
| CRISPLD1 | 3809.11608 | 6706.93175 | 3794.26871 | 6775.22124 | 52.9983066 | 129.258795 |
| CD151 | 4505.04844 | 7924.8738 | 4501.15651 | 7854.30052 | 25.8032474 | 250.307312 |
| AKR1B1 | 15003.9335 | 27257.967 | 14994.4347 | 26561.7342 | 609.128016 | 1245.60734 |
| ERGIC1 | 6776.31783 | 11881.1793 | 6832.31716 | 11957.3667 | 142.124543 | 449.679643 |
| MAP1B | 7613.71361 | 12653.9661 | 7637.37722 | 12633.0199 | 128.282292 | 326.55481 |
| EPGN | 17613.2085 | 10436.5055 | 17793.4711 | 10505.8287 | 545.605495 | 271.775438 |
| CDCP1 | 11454.2487 | 6413.57736 | 11512.2062 | 6284.34334 | 251.580521 | 294.333079 |
| PTPN12 | 7696.22514 | 4476.67841 | 7605.81901 | 4500.28419 | 158.066955 | 49.6302855 |
| TGFB2 | 2562.29929 | 5512.81677 | 2550.95444 | 5600.88632 | 181.261718 | 196.651089 |
| PLK2 | 1542.18198 | 3230.2274 | 1512.13897 | 3249.39376 | 59.6533998 | 40.1504886 |
| PIEZO2 | 4586.14647 | 8121.76892 | 4528.06324 | 8222.62669 | 127.434581 | 228.730395 |
| GPR37 | 3571.45465 | 1676.14846 | 3608.01159 | 1694.35618 | 104.137861 | 79.4801757 |
| COL5A1 | 718.316784 | 2133.51993 | 697.984148 | 2038.34843 | 43.9667124 | 192.908764 |
| PTGES | 2316.331 | 1001.81478 | 2324.37362 | 999.03003 | 14.2793418 | 36.0318014 |
| GNG11 | 4126.35433 | 1950.79437 | 4129.33951 | 1952.57076 | 66.004245 | 128.746673 |
| SPRED1 | 1999.51767 | 815.120385 | 1998.1377 | 803.601375 | 32.0490643 | 22.4235308 |
| PRAME | 9238.61122 | 5407.51631 | 9357.01995 | 5434.22746 | 246.071358 | 96.8077694 |
| RSU1 | 2818.86296 | 5229.4847 | 2782.53811 | 5229.84228 | 76.7904717 | 120.512624 |
| MAP3K21 | 623.205965 | 1697.65801 | 609.523655 | 1701.40132 | 40.8889227 | 37.9329708 |
| LSS | 3211.66107 | 5708.32299 | 3203.81445 | 5784.45619 | 27.5129005 | 133.560747 |
| ZNF185 | 1431.40967 | 2975.7997 | 1424.79946 | 2963.8444 | 27.8207588 | 72.8737599 |
| SYNPO | 6467.54644 | 10668.015 | 6482.51577 | 10551.2714 | 36.280907 | 296.227652 |
| IFITM2 | 10560.7151 | 6386.13435 | 10537.2959 | 6433.32561 | 100.881906 | 186.918967 |
| DAPK1 | 1568.91768 | 3171.30471 | 1546.72356 | 3178.53234 | 51.0883514 | 16.5876454 |
| RGS2 | 1527.08001 | 466.066342 | 1486.02891 | 463.872268 | 131.569147 | 22.5540366 |
| SPRY2 | 991.004045 | 259.712651 | 976.918239 | 254.799231 | 37.1389947 | 12.3635727 |
| FOSL2 | 7019.89343 | 11872.5833 | 6949.18072 | 11831.1385 | 183.75896 | 384.972883 |
| MYL6 | 13111.9293 | 19990.1917 | 13184.8918 | 19989.8987 | 171.953271 | 304.361942 |
| ANKRD1 | 1051.71649 | 2477.49793 | 1057.42279 | 2486.72728 | 31.559123 | 144.685421 |
| CAPN2 | 11466.1842 | 17609.0407 | 11371.2851 | 17615.1985 | 178.960653 | 226.82794 |
| NQO1 | 98820.9694 | 65523.7218 | 98833.0712 | 66189.0971 | 1696.75137 | 1619.5072 |
| DMD | 2447.76981 | 1108.97165 | 2438.62412 | 1104.91279 | 64.3849838 | 33.6378533 |
| EEF1A2 | 21693.272 | 34780.5478 | 21644.2292 | 35215.1215 | 278.497744 | 1368.82556 |
| FOS | 677.872495 | 106.928342 | 702.784589 | 100.21294 | 48.4694046 | 11.9854343 |
| ABCB1 | 236.527191 | 902.831101 | 245.889676 | 908.115198 | 16.9334065 | 35.9329666 |
| TIMP1 | 2976.24627 | 1453.54348 | 2966.22644 | 1457.73668 | 83.7959734 | 32.9301279 |
| PLD3 | 9366.88077 | 15610.1586 | 9398.23385 | 15748.2363 | 373.557553 | 368.268012 |
| TTC7A | 2937.80287 | 5307.02451 | 2905.96889 | 5343.31263 | 113.342521 | 73.6423983 |
| FMNL2 | 6374.3144 | 3778.43656 | 6371.14554 | 3775.03178 | 88.2238271 | 69.7608782 |
| PLEKHA6 | 2254.98179 | 1018.38242 | 2275.20842 | 1018.65937 | 37.2065836 | 39.7019791 |
| LAMB2 | 7338.15178 | 13392.1672 | 7287.27584 | 13554.8494 | 440.297312 | 465.983259 |
| KIF1C | 5895.179 | 9799.68121 | 5913.98686 | 9730.9864 | 127.252468 | 319.287402 |
| JUND | 2958.2714 | 5776.95706 | 2896.24993 | 5933.1821 | 155.91028 | 275.11354 |
| BASP1 | 23621.0143 | 35276.1027 | 23543.2075 | 35457.8176 | 200.345546 | 889.33812 |
| ABCA3 | 1806.33126 | 3704.3175 | 1779.68686 | 3742.14355 | 115.335962 | 108.138975 |
| SPRY4 | 1387.1656 | 459.286789 | 1352.87916 | 448.540582 | 95.2927706 | 21.0147786 |
| PEA15 | 5951.06187 | 10075.0538 | 5909.3431 | 10219.9585 | 184.928817 | 339.082473 |
| SNHG3 | 3558.14164 | 1868.3812 | 3557.13918 | 1905.71038 | 2.48123604 | 72.1301594 |
| CYTH3 | 1832.79151 | 3459.6261 | 1835.68871 | 3427.72103 | 32.4606178 | 81.1475223 |
| DUSP6 | 656.649637 | 118.746255 | 641.338942 | 118.50863 | 40.6285564 | 6.14627288 |
| SYNGR3 | 159.860973 | 737.052594 | 165.135177 | 762.049313 | 9.67046627 | 61.4586955 |
| PDE8A | 6568.89222 | 3711.07345 | 6567.00354 | 3623.16268 | 92.1645649 | 189.753689 |
| ICAM1 | 2265.1583 | 1071.0962 | 2253.32709 | 1058.67709 | 33.4343162 | 22.1711293 |
| DUSP4 | 5291.31247 | 2554.13705 | 5443.7003 | 2505.72147 | 326.323846 | 150.909465 |
| FSIP2 | 1144.12998 | 314.808662 | 1105.06156 | 305.86513 | 68.8410901 | 44.0323342 |
| PPP1R13L | 1637.23125 | 3207.19217 | 1612.41157 | 3216.66281 | 49.3054223 | 119.316124 |
| PLCD3 | 1782.2011 | 3395.34344 | 1775.86779 | 3352.10124 | 33.4915281 | 122.196272 |
| CD44 | 9815.35572 | 6123.19239 | 9648.01111 | 6134.2324 | 290.754403 | 72.402105 |
| BMP6 | 2870.60699 | 1461.41558 | 2897.22183 | 1483.56476 | 63.7603848 | 39.9198552 |
| NPR3 | 2926.45163 | 5122.6113 | 2904.78504 | 5029.28051 | 51.8907528 | 182.571101 |
| LATS2 | 1826.67624 | 3396.39597 | 1814.56677 | 3400.00809 | 29.8247599 | 97.5145651 |
| SPRED2 | 4594.08585 | 2541.53919 | 4621.86477 | 2513.9408 | 78.2753392 | 118.748108 |
| HACD2 | 4412.99204 | 7544.90757 | 4337.67865 | 7410.31312 | 152.95034 | 266.663109 |
| ERBB2 | 4637.74151 | 7412.55668 | 4644.69075 | 7371.23677 | 23.8915533 | 172.260811 |
| DNMBP | 4345.69014 | 2227.9814 | 4384.72298 | 2260.31576 | 240.764178 | 85.2136691 |
| CAV2 | 2825.6986 | 5031.22228 | 2871.94103 | 5033.75255 | 140.327893 | 52.4159132 |
| COL8A1 | 6280.51178 | 9614.8827 | 6263.61566 | 9629.53223 | 92.3460115 | 87.1642677 |
| MYEOV | 1403.4887 | 454.657499 | 1350.93537 | 483.063748 | 96.9840764 | 52.9023105 |
| DUSP3 | 3241.81617 | 5371.77507 | 3260.72241 | 5358.84737 | 47.7641124 | 88.3279709 |
| TNFAIP1 | 4889.23114 | 7838.38239 | 4918.53204 | 7762.88691 | 108.496403 | 160.984714 |
| RIN1 | 1444.83566 | 565.664596 | 1438.26196 | 563.198155 | 25.9050462 | 38.5715035 |
| DLC1 | 993.850721 | 2052.62321 | 991.771149 | 2047.78737 | 17.2574108 | 12.2610542 |
| AJUBA | 5553.63699 | 9354.80659 | 5426.0951 | 9222.68984 | 255.465522 | 252.071932 |
| CREM | 2406.36337 | 1207.8665 | 2398.37629 | 1212.88651 | 49.0302138 | 15.2593359 |
| SCARA3 | 526.839685 | 1315.06223 | 529.008618 | 1322.13795 | 9.85905441 | 31.1119441 |
| TRIM16L | 4209.9101 | 6677.77401 | 4229.23869 | 6714.01845 | 47.0987561 | 83.4704908 |
| DHCR24 | 15162.8205 | 23093.0845 | 14992.7379 | 23069.6386 | 498.283687 | 418.234187 |
| PHLDB1 | 1903.92421 | 3491.4374 | 1897.1409 | 3435.62161 | 15.0173799 | 131.611712 |
| MFSD12 | 1841.18138 | 3330.66984 | 1845.63041 | 3342.91894 | 48.7291713 | 31.8253605 |
| LUCAT1 | 2106.76331 | 902.498973 | 2118.73317 | 917.413305 | 140.45064 | 39.4399864 |
| CALD1 | 5556.56252 | 9802.53996 | 5650.60306 | 9812.60313 | 232.281257 | 530.8724 |
| PLAUR | 2496.29405 | 1171.77738 | 2513.71222 | 1134.29688 | 41.2632463 | 93.9599495 |
| TRIB3 | 2746.38676 | 1343.56648 | 2742.69037 | 1382.3187 | 54.3569518 | 93.4221664 |
| TUBB2A | 838.150156 | 1831.29622 | 824.67767 | 1831.73641 | 45.5397813 | 21.0479164 |
| AFAP1 | 1563.16174 | 2954.84246 | 1557.94921 | 2959.32978 | 68.1799654 | 47.6155128 |
| ARHGAP12 | 4110.804 | 2356.07346 | 4143.19244 | 2341.95625 | 110.360343 | 52.4474537 |
| TUBB6 | 3224.46369 | 5831.85775 | 3268.48608 | 5706.97196 | 86.2488526 | 357.151511 |
| STIM1 | 3243.52861 | 5241.97734 | 3222.05612 | 5249.36797 | 46.9594475 | 30.9769696 |
| HLA-C | 7930.22776 | 12921.8643 | 7955.94026 | 12685.9488 | 270.219941 | 538.486988 |
| XDH | 1248.75513 | 495.618014 | 1235.08741 | 492.799715 | 27.8131138 | 10.0395121 |
| F2RL2 | 4297.99945 | 6811.41498 | 4260.55507 | 6853.73859 | 65.413706 | 154.786297 |
| TIMP2 | 5731.48584 | 8863.71511 | 5713.7763 | 8887.44415 | 81.1762763 | 221.989611 |
| TNKS1BP1 | 6492.59074 | 10159.3976 | 6507.47807 | 9973.91201 | 80.0828243 | 358.807519 |
| CAMK1D | 1681.15272 | 3441.83237 | 1659.02639 | 3484.99594 | 62.9333094 | 259.342603 |
| NR4A3 | 581.267076 | 1411.38656 | 601.603594 | 1397.28611 | 39.5118683 | 30.4878889 |
| LTBP4 | 3740.96726 | 7078.29889 | 3826.91171 | 6844.15553 | 204.580558 | 478.31844 |
| FBXO27 | 8112.09413 | 4817.03212 | 8232.38841 | 4839.66671 | 226.368234 | 245.260972 |
| SOCS3 | 2014.33614 | 817.499639 | 2047.86821 | 787.881493 | 151.191216 | 65.5376111 |
| LRATD2 | 4810.90125 | 2933.99797 | 4831.16401 | 2957.78462 | 46.7570254 | 57.9405847 |
| TSC22D3 | 1136.56615 | 2367.21215 | 1165.54712 | 2417.65722 | 67.5047223 | 95.2278254 |
| INF2 | 3096.72656 | 5112.16796 | 3102.61289 | 5163.59029 | 28.877073 | 158.879242 |
| JUP | 38918.3016 | 27664.2482 | 38868.9721 | 27837.6966 | 639.060776 | 425.79193 |
| TBC1D8 | 3948.97848 | 1994.53492 | 4035.31199 | 1951.56952 | 224.80426 | 126.599771 |
| CERCAM | 1394.97582 | 2803.26171 | 1407.48935 | 2744.8856 | 65.1727127 | 155.523436 |
| MLX | 4167.96431 | 2461.35587 | 4177.34392 | 2458.20758 | 88.4884108 | 47.5664267 |
| NT5C2 | 1950.9763 | 3565.72386 | 1932.65762 | 3579.77151 | 45.4440014 | 169.789629 |
| TRAM1 | 5340.1248 | 9697.52958 | 5381.38788 | 9628.70721 | 323.255161 | 561.168404 |
| ANXA2 | 39882.0639 | 56834.4062 | 39763.0144 | 57201.9593 | 636.798985 | 1423.28476 |
| YWHAH | 4456.41723 | 6914.85369 | 4457.68968 | 6935.57647 | 91.9747787 | 65.6517162 |
| FOXL1 | 1314.26234 | 524.748172 | 1282.90266 | 524.862932 | 70.5064542 | 9.1629079 |
| HLA-A | 8791.60973 | 13332.7292 | 8764.00303 | 13484.398 | 120.992709 | 439.667123 |
| GPSM3 | 1799.8089 | 847.79307 | 1797.28518 | 867.726414 | 24.5424667 | 37.3279158 |
| EFNA1 | 4466.36741 | 7383.20344 | 4524.07094 | 7372.73904 | 105.69991 | 332.216856 |
| SLC4A2 | 6505.29327 | 10257.0711 | 6402.82845 | 10174.1962 | 271.601142 | 153.682772 |
| PMEPA1 | 9083.53703 | 13828.5962 | 9158.17555 | 13653.4814 | 162.001462 | 466.496847 |
| CPA4 | 4979.1147 | 7616.00561 | 5008.78033 | 7583.12349 | 95.6811476 | 81.2635569 |
| PRKAB2 | 2206.78634 | 3727.24085 | 2215.82472 | 3742.14355 | 20.0113235 | 58.7438005 |
| NBR1 | 7030.58666 | 4319.72867 | 7054.99271 | 4315.35515 | 67.8411281 | 204.622096 |
| VEGFC | 2095.46729 | 1018.18694 | 2065.1498 | 1036.22246 | 66.3163946 | 48.984547 |
| RASD1 | 805.798743 | 221.608724 | 835.830518 | 217.225151 | 58.2976952 | 23.680903 |
| COL4A3 | 1599.2814 | 3059.9003 | 1613.34728 | 3010.11919 | 90.6483271 | 103.531973 |
| TLE6 | 665.757647 | 170.437957 | 664.776831 | 174.597803 | 26.9490407 | 16.3906016 |
| SAMD4A | 3317.7268 | 5485.33635 | 3301.96837 | 5464.68028 | 129.234784 | 119.604032 |
| MRTFA | 2618.36257 | 4490.29029 | 2629.68168 | 4439.61241 | 47.9112299 | 191.218701 |
| VAT1 | 9168.25824 | 14328.9157 | 9137.50813 | 14458.5577 | 191.568169 | 626.556446 |
| FOSL1 | 1708.95481 | 776.16865 | 1708.59308 | 799.08676 | 52.9843908 | 43.813546 |
| ORMDL3 | 2168.46145 | 3786.03629 | 2209.7142 | 3751.26995 | 82.0007748 | 93.9742354 |
| ANKRD28 | 4614.86301 | 2798.46795 | 4563.05149 | 2788.39922 | 126.049744 | 17.9833489 |
| TMEM131 | 9502.48089 | 6325.75701 | 9565.39995 | 6309.28272 | 156.907773 | 127.201904 |
| APLP1 | 4213.77926 | 6742.60434 | 4292.55451 | 6745.96267 | 149.521839 | 131.441814 |
| SCEL | 2195.24514 | 1032.34079 | 2200.52224 | 1009.36126 | 70.1162856 | 84.5918949 |
| RIPK4 | 1973.11302 | 995.813202 | 1961.47462 | 992.190554 | 29.7898292 | 6.36491063 |
| RHOBTB1 | 816.983409 | 1766.39877 | 803.593854 | 1798.85909 | 24.7486524 | 93.1343396 |
| LAT2 | 2752.58046 | 1483.63814 | 2778.49536 | 1463.86374 | 76.1240418 | 51.1387706 |
| SERINC3 | 10686.0736 | 7199.33626 | 10736.171 | 7207.17825 | 124.715405 | 170.313697 |
| ID3 | 4778.38055 | 12246.5352 | 4898.37018 | 122921.665 | 317.945504 | 756.438762 |
| AGRN | 16475.5789 | 26744.941 | 16627.1959 | 26372.1204 | 440.061131 | 1631.64033 |
| FRMD4A | 556.364264 | 121.079456 | 535.514669 | 119.637283 | 40.1495694 | 3.44194741 |
| CCDC85C | 1542.07299 | 2808.83589 | 1543.37077 | 2764.04327 | 42.7411367 | 82.484454 |
| SDC4 | 9626.85618 | 14275.1793 | 9476.95742 | 14346.2535 | 312.535983 | 143.155312 |
| SLC7A5 | 12686.1298 | 18937.0034 | 12579.0761 | 18937.1463 | 493.80202 | 203.904707 |
| EML2 | 1909.45478 | 944.870805 | 1898.07805 | 949.197691 | 57.7251214 | 20.4742673 |
| SMPD1 | 2225.00833 | 3889.37281 | 2215.82472 | 3951.14937 | 51.2305329 | 173.960933 |
| TAPBP | 10782.8709 | 17323.6698 | 10646.1482 | 17719.7014 | 503.4518 | 698.881254 |
| RUSC2 | 2250.55858 | 3852.80351 | 2243.32202 | 3806.94865 | 59.9641892 | 124.809121 |
| RAI14 | 5701.84305 | 8717.8994 | 5690.45079 | 8762.86668 | 182.498253 | 111.01839 |
| CAB39 | 3495.18279 | 2066.7638 | 3514.37576 | 2050.74934 | 37.9075261 | 38.9737472 |
| ARHGEF17 | 1488.20757 | 2646.77475 | 1495.81747 | 2653.46465 | 19.9061285 | 26.7941419 |
| NOTCH2 | 1739.20877 | 3326.88102 | 1750.24086 | 3211.40966 | 52.2222318 | 231.318183 |
| GRN | 5782.61935 | 8966.92078 | 5760.4273 | 8973.16002 | 98.322591 | 336.41631 |
| TPM2 | 663.096308 | 1468.28388 | 648.05956 | 1458.76981 | 34.6094211 | 40.2081877 |
| GAA | 2562.91433 | 4723.5932 | 2634.80992 | 4704.97935 | 166.629834 | 225.63416 |
| IRF2BPL | 8301.71141 | 4733.73948 | 8161.9822 | 4757.8179 | 492.650103 | 247.468795 |
| FADS2 | 21388.3369 | 14575.2242 | 21236.3235 | 14531.9094 | 367.490308 | 509.281931 |
| PITPNC1 | 4823.00046 | 2833.83182 | 4831.16401 | 2887.57904 | 16.8469949 | 167.972134 |
| GBP1 | 836.577535 | 1689.48558 | 835.830518 | 1706.09808 | 9.37732601 | 30.2177547 |
| SELENOM | 2559.23855 | 4149.89382 | 2566.31586 | 4114.92927 | 34.18387 | 79.0083518 |
| ADGRB2 | 1964.71297 | 3377.54223 | 1946.70759 | 3408.67358 | 68.2752855 | 67.4187831 |
| CKB | 1041.28972 | 2188.26969 | 1021.46264 | 2124.10972 | 55.6152514 | 151.897242 |
| AMOTL2 | 6359.40194 | 10179.4485 | 6304.89945 | 9916.35067 | 150.772331 | 526.647599 |
| KIAA0319 | 4573.48279 | 7156.63812 | 4567.60834 | 7041.66991 | 33.6690564 | 286.636417 |
| TRAM2 | 2560.1442 | 4284.89066 | 2543.27374 | 4250.56782 | 39.5853331 | 175.965874 |
| ERO1A | 10848.1172 | 6903.83777 | 10802.6229 | 6880.27245 | 396.344701 | 262.280869 |
| THSD4 | 10579.8917 | 6498.54501 | 10671.4175 | 6691.53827 | 358.308675 | 350.819982 |
| HEATR5A | 1504.04718 | 2743.25537 | 1515.18579 | 2674.90907 | 29.2585883 | 124.829707 |
| MYORG | 1653.18718 | 2860.80033 | 1660.97018 | 2848.32036 | 16.8996484 | 57.7138297 |
| 4-Mar | 3156.23632 | 1788.58459 | 3116.44642 | 1774.24348 | 125.321044 | 32.259941 |
| STK38 | 3911.49269 | 6183.18861 | 3902.75868 | 6084.06189 | 61.7850893 | 232.340679 |
| MAP4 | 14846.3981 | 20745.5937 | 14789.1992 | 20614.0797 | 241.436966 | 323.311253 |
| MAP2 | 12459.7334 | 17576.5472 | 12378.4176 | 17437.6984 | 210.528501 | 296.920532 |
| PLLP | 3747.76785 | 2202.37877 | 3702.20696 | 2240.84399 | 97.3601004 | 81.901729 |
| GDF15 | 375.483735 | 49.5237018 | 377.314677 | 48.5567853 | 27.8418496 | 3.15279558 |
| ZYX | 6072.80414 | 9335.82707 | 6061.03704 | 9358.02896 | 48.1912058 | 401.342921 |
| NPAS2 | 1378.77899 | 627.366654 | 1362.36521 | 628.191653 | 44.9783207 | 16.3939832 |
| AKR1C2 | 6840.38489 | 11587.4904 | 6811.04683 | 11858.1868 | 317.822397 | 735.559538 |
| B2M | 10248.6659 | 14715.9257 | 10257.3899 | 14733.4443 | 275.811243 | 198.281756 |
| HPS5 | 4009.20568 | 6511.93161 | 4026.61006 | 6599.59031 | 140.941043 | 283.283885 |
| MYOCD | 193.020042 | 631.011053 | 191.463502 | 637.436948 | 6.05207441 | 26.8096603 |
| KLF7 | 1134.43906 | 2215.69875 | 1138.66465 | 2244.97648 | 19.8457153 | 139.083115 |
| DCBLD2 | 7915.83982 | 4943.70264 | 7769.03401 | 4944.51411 | 338.929612 | 178.534589 |
| TMEM156 | 822.506493 | 273.559632 | 834.858622 | 281.805601 | 24.7667783 | 31.6026327 |
| RHPN2 | 3463.24331 | 2117.28115 | 3452.4773 | 2117.06458 | 29.6203279 | 5.68161516 |
| MTCH1 | 7013.598 | 10565.2911 | 7177.56113 | 10534.8327 | 288.210705 | 122.001602 |
| TMEM132A | 2673.80614 | 4390.63217 | 2627.03476 | 4344.50302 | 93.4215274 | 131.050405 |
| IL4R | 1698.16693 | 858.472257 | 1701.27636 | 867.726414 | 7.92836793 | 25.5374792 |
| COL18A1 | 775.246571 | 1755.98152 | 764.882114 | 1695.23773 | 48.0137159 | 151.888168 |
| RELB | 1226.12788 | 2450.46654 | 1224.39401 | 2553.88028 | 41.3090501 | 190.203026 |
| SIPA1L2 | 4117.98488 | 2418.56745 | 4156.79898 | 2444.36924 | 160.101703 | 96.7983302 |
| IL6R | 2293.53686 | 1236.25201 | 2255.54305 | 1249.04582 | 80.3331295 | 33.1324533 |
| TRIM37 | 2964.38216 | 5044.65217 | 2964.75247 | 4897.00345 | 111.68285 | 270.33128 |
| CABLES1 | 990.263557 | 1911.80494 | 1003.29221 | 1936.23932 | 36.9439587 | 56.1238294 |
| FN1 | 1983.5628 | 3787.5001 | 2013.76841 | 3726.87908 | 175.45781 | 143.8104 |
| CDV3 | 8288.33336 | 12226.9946 | 8286.38488 | 12184.6537 | 289.857325 | 201.983397 |
| UCHL1 | 6049.12821 | 8711.26152 | 6042.27713 | 8692.89015 | 59.0218691 | 101.325017 |
| MBP | 2168.54241 | 1185.69928 | 2188.32741 | 1189.60091 | 46.1324283 | 8.7344267 |
| PARVA | 930.530833 | 1781.57676 | 934.908463 | 1776.97172 | 10.2634339 | 35.7335998 |
| PGK1 | 22883.3158 | 16698.9733 | 23096.2109 | 16678.1145 | 381.374001 | 170.720728 |
| PALM | 1235.10078 | 2306.52293 | 1268.27656 | 2321.42759 | 70.5089874 | 32.0613039 |
| HSP90AA1 | 149246.409 | 99581.3035 | 147434.672 | 99435.1064 | 7567.9106 | 2789.58945 |
| RGP1 | 2758.66614 | 1587.59203 | 2759.66036 | 1597.04487 | 19.3853701 | 59.334437 |
| ECE1 | 8124.77208 | 11685.6255 | 8160.58989 | 11677.3196 | 73.2590929 | 314.161061 |
| DYNC1H1 | 76924.6553 | 54384.1678 | 77272.5314 | 53701.5799 | 616.038952 | 2154.20798 |
| GPR3 | 494.362484 | 116.802885 | 503.086236 | 111.736708 | 23.7522154 | 10.7600555 |
| CTSA | 4683.56058 | 6891.1034 | 4701.06072 | 6932.41779 | 42.0489348 | 110.141207 |
| ECT2 | 10061.5333 | 6258.69403 | 9917.22629 | 6199.77167 | 411.126453 | 333.073026 |
| TP53I3 | 1971.02804 | 3378.69083 | 2017.65599 | 3327.65447 | 90.4649869 | 93.6654875 |
| NISCH | 2597.93005 | 4269.20528 | 2619.25959 | 4204.23472 | 81.0332268 | 162.176536 |
| C3 | 552.214042 | 1232.15043 | 551.065004 | 1231.72532 | 9.43126265 | 64.2075491 |
| TES | 4081.30878 | 6595.30537 | 3985.32627 | 6633.68337 | 200.47543 | 233.174468 |
| TOR4A | 2611.19114 | 1507.3389 | 2596.96795 | 1502.23796 | 67.2417976 | 12.6568229 |
| CLSTN3 | 3039.26553 | 5016.40487 | 3061.47225 | 5094.81043 | 82.8660648 | 259.443846 |
| SLC2A8 | 654.284714 | 1371.98565 | 657.001663 | 1396.14452 | 22.1411693 | 44.2069366 |
| MTHFD2L | 947.019904 | 374.614452 | 939.491348 | 374.566612 | 46.8370838 | 6.84667619 |
| VSIR | 635.977301 | 1447.56059 | 650.005802 | 1457.1698 | 59.7091751 | 71.2561271 |
| AC008875.3 | 1084.56371 | 441.867416 | 1084.61603 | 422.708402 | 37.8056318 | 39.7590467 |
| SQLE | 4618.41297 | 7096.81098 | 4621.36525 | 7209.13293 | 80.5604923 | 286.853528 |
| HIF1A | 11252.4966 | 7845.67702 | 11251.6394 | 7787.68187 | 111.103605 | 224.702189 |
| PLXNB2 | 5198.16046 | 8082.94995 | 5196.99116 | 7847.52859 | 5.03600081 | 423.471834 |
| FOXC2 | 1511.52713 | 701.702545 | 1500.1309 | 688.075343 | 48.7285123 | 56.8214163 |
| CCDC68 | 1191.41788 | 541.391783 | 1179.32898 | 541.753736 | 28.1722069 | 10.4816125 |
| AP1M2 | 1880.70085 | 3081.37537 | 1886.45004 | 3072.85524 | 21.5973516 | 44.7337823 |
| C1orf198 | 1850.77137 | 3075.82878 | 1852.9703 | 3060.90861 | 29.3725866 | 84.2303904 |
| ACOX2 | 318.188525 | 12.401856 | 341.791412 | 11.7419001 | 45.4372117 | 4.86494771 |
| LAMP3 | 1024.22156 | 432.145379 | 1028.2545 | 435.624492 | 32.4902842 | 13.1537136 |
| NHSL1 | 1615.07469 | 830.61511 | 1613.9392 | 826.174447 | 13.2916001 | 21.1532125 |
| ELK3 | 3360.52688 | 2013.63858 | 3368.59137 | 1987.72883 | 114.489874 | 49.9958422 |
| PCDH9 | 548.922141 | 1265.82974 | 575.915834 | 1252.80551 | 52.2604809 | 43.2861107 |
| LRP8 | 2364.48637 | 1333.12141 | 2358.9368 | 1352.66689 | 28.865284 | 58.3785039 |
| HMGB3 | 10283.5546 | 7142.00535 | 10261.0787 | 7075.66897 | 256.848528 | 152.628745 |
| COL11A2 | 163.93142 | 604.684393 | 162.306624 | 621.888143 | 10.2664978 | 81.9783488 |
| CNN1 | 10.6839006 | 247.398576 | 7.68070589 | 247.949542 | 5.97768724 | 16.9885547 |
| OAS1 | 3234.33012 | 1919.99712 | 3270.65199 | 1943.30454 | 82.2278051 | 91.2151438 |
| APEH | 6785.01959 | 4563.08427 | 6747.50013 | 4506.71389 | 101.116321 | 147.835053 |
| DYNLL2 | 2928.69873 | 4562.04338 | 2882.18489 | 4519.12908 | 82.1646266 | 117.066756 |
| NRG1 | 1925.6273 | 3396.12722 | 1868.33171 | 3390.47546 | 107.211647 | 169.481012 |
| S100A10 | 15371.2937 | 22557.2924 | 15336.5181 | 22632.6276 | 128.950484 | 1151.54694 |
| SPRY1 | 1446.6578 | 689.609958 | 1412.2898 | 700.991433 | 69.6829867 | 38.4843528 |
| SNORC | 204.224959 | 616.393086 | 204.498794 | 624.669083 | 2.91524186 | 36.2291135 |
| LGALS3BP | 35797.2028 | 49940.1508 | 35598.6049 | 49473.3217 | 554.66508 | 1847.63138 |
| SLC22A23 | 2120.72526 | 1093.02032 | 2177.04693 | 1071.09228 | 122.855703 | 61.4706538 |
| DBN1 | 5652.92541 | 8046.71655 | 5624.36187 | 8101.75129 | 55.8904953 | 134.988084 |
| CDKN2A | 1123.74244 | 2126.62688 | 1125.45551 | 2162.32663 | 77.2386429 | 74.8543628 |
| B4GALT5 | 5423.51676 | 7828.13331 | 5404.71338 | 7877.56357 | 49.9917471 | 199.803795 |
| FAM83H | 4407.30527 | 6387.99513 | 4406.80501 | 6356.57717 | 34.0263751 | 97.4812287 |
| TIAM1 | 1215.27566 | 553.148621 | 1238.51383 | 557.88647 | 54.8839199 | 27.3999129 |
| TPRA1 | 1362.23665 | 2437.45806 | 1329.72221 | 2476.39605 | 73.0512937 | 76.2919092 |
| PHC2 | 4535.22848 | 2952.58896 | 4550.81824 | 2965.06327 | 105.205033 | 52.4792434 |
| TPBG | 4373.73502 | 2718.2314 | 4457.68968 | 2734.68852 | 208.458978 | 30.5150019 |
| IGFBP3 | 8676.50104 | 21509.6518 | 8593.74981 | 22445.5345 | 506.62264 | 1745.09392 |
| PXDN | 30709.7913 | 22602.4278 | 30564.1839 | 22418.8098 | 496.092102 | 536.896588 |
| EDEM2 | 2087.47278 | 3316.68581 | 2063.3351 | 3308.86743 | 44.72119 | 34.6241693 |
| ANKLE2 | 3222.50896 | 4800.08085 | 3243.21679 | 4805.8071 | 41.7874668 | 10.1629283 |
| EHD2 | 6564.15797 | 9284.04665 | 6497.00277 | 9275.27542 | 129.907093 | 145.213502 |
| HIST1H1C | 2915.99522 | 5212.02215 | 2893.33425 | 5297.9453 | 212.112564 | 347.609669 |
| EDN1 | 501.338414 | 1100.7112 | 510.227821 | 1079.08061 | 16.7120772 | 49.0605884 |
| B4GALT1 | 7864.55866 | 11316.2235 | 7852.56169 | 11404.9075 | 56.5052016 | 427.953725 |
| PTGS2 | 652.474266 | 224.711464 | 649.019648 | 221.216109 | 26.5260481 | 8.60950133 |
| RTL8C | 5479.66749 | 7930.64435 | 5475.78331 | 7863.32975 | 31.3977902 | 249.72779 |
| KCTD15 | 1078.54909 | 1914.91142 | 1086.14366 | 1899.83943 | 21.6005937 | 26.3790563 |
| TSKU | 2642.74729 | 4561.59474 | 2668.82628 | 4547.63789 | 92.0750003 | 343.106986 |
| PLXNB1 | 720.336484 | 1402.92602 | 726.387213 | 1413.72477 | 15.442752 | 26.7704914 |
| VLDLR | 506.646869 | 1148.01188 | 497.325707 | 1143.66726 | 45.3414018 | 47.6384294 |
| GLI1 | 286.047306 | 786.985444 | 264.355699 | 800.797584 | 38.3005845 | 24.7120419 |
| CUEDC1 | 2032.5696 | 3309.27743 | 2054.65994 | 3374.18002 | 46.8650815 | 128.816181 |
| EDEM1 | 3840.32053 | 2403.53441 | 3816.77908 | 2348.28879 | 59.5039869 | 113.338233 |
| DCLK1 | 294.035244 | 39.4809863 | 298.372057 | 41.0966502 | 18.6243173 | 3.94159976 |
| ALDH3B1 | 3128.54921 | 4972.47143 | 3137.56836 | 4865.84338 | 45.0238589 | 277.550383 |
| TPCN1 | 3480.43263 | 2211.66962 | 3447.31494 | 2223.28089 | 78.9690611 | 29.5077682 |
| RGL2 | 2093.16445 | 1193.05892 | 2114.84559 | 1197.67381 | 48.9740436 | 29.6376065 |
| EPHA2 | 4950.06423 | 3252.07718 | 4994.37901 | 3250.15793 | 147.375809 | 73.6383194 |
| CITED4 | 2929.0353 | 1630.85575 | 2881.6715 | 1642.69182 | 188.410253 | 79.3471342 |
| CXCL1 | 747.169296 | 279.843938 | 753.120706 | 294.721691 | 13.3878194 | 27.3291359 |
| WWC1 | 3657.69499 | 5367.42263 | 3664.6568 | 5394.96878 | 53.6792417 | 95.1433973 |
| COL4A5 | 3395.59897 | 5244.27153 | 3421.07375 | 5190.67798 | 108.368522 | 201.633668 |
| HAS3 | 786.162943 | 310.709302 | 783.348137 | 306.837558 | 35.904106 | 8.59012782 |
| CD81 | 4789.57613 | 6802.92038 | 4773.83815 | 6829.08907 | 76.2310043 | 64.8893428 |
| FERMT1 | 1876.25209 | 1066.71201 | 1867.52548 | 1068.24928 | 18.1656632 | 7.38723936 |
| TSPAN5 | 1636.00604 | 885.474445 | 1617.74868 | 890.036024 | 47.1520921 | 8.35181287 |
| MVD | 3080.7061 | 4793.92568 | 3077.02258 | 4828.81733 | 151.063011 | 89.8973433 |
| NECTIN2 | 3074.13166 | 4630.22282 | 3057.54786 | 4665.85405 | 71.0135847 | 110.271438 |
| NPTX1 | 514.2884 | 154.169117 | 513.161062 | 147.736602 | 17.5535332 | 15.1107025 |
| NPTXR | 1455.52909 | 2533.02295 | 1468.53478 | 2486.93443 | 26.2846296 | 153.914432 |
| WIPF1 | 3277.76149 | 4974.00851 | 3278.29013 | 4926.90126 | 80.404645 | 172.9342 |
| PPM1H | 1416.42976 | 2378.35721 | 1427.71515 | 2355.5001 | 26.302835 | 75.1447497 |
| SEZ6L2 | 3927.38395 | 6060.38067 | 3970.19496 | 6036.03954 | 172.663542 | 228.408213 |
| NABP1 | 737.609354 | 1447.77697 | 740.584715 | 1486.43681 | 20.5897291 | 82.8466729 |
| DCLK2 | 982.110459 | 1738.57231 | 985.320194 | 1748.04427 | 10.2408543 | 25.2932199 |
| CHPF | 3448.39262 | 5282.77246 | 3383.69648 | 5291.12815 | 180.358369 | 44.6458944 |
| HACD1 | 779.05605 | 1476.86249 | 779.591648 | 1457.73668 | 10.4016092 | 63.1689236 |
| PLS3 | 38686.2449 | 51894.7428 | 38440.4287 | 51498.2072 | 1151.30315 | 737.597682 |
| ENTPD6 | 4724.05068 | 3221.59274 | 4692.87385 | 3223.34405 | 57.0365345 | 14.0860702 |
| STAT3 | 27090.9849 | 36998.4197 | 27112.3454 | 37202.7625 | 440.053644 | 1270.26903 |
| FZD2 | 1134.33572 | 1962.41252 | 1127.3993 | 1961.90075 | 36.8765502 | 15.8675715 |
| SYNPO2 | 1424.99005 | 2417.67431 | 1446.18118 | 2411.78627 | 38.1261002 | 101.363484 |
| ZNF281 | 3651.33992 | 5652.89318 | 3588.39866 | 5522.50214 | 110.326885 | 284.956922 |
| USP2 | 383.672186 | 863.935264 | 381.955109 | 857.158704 | 11.1254549 | 13.2663021 |
| RAB27B | 1232.62805 | 598.009251 | 1245.23444 | 601.572378 | 55.7141491 | 26.9229566 |
| SLC16A14 | 2992.04418 | 1784.45763 | 3002.18659 | 1837.92598 | 65.0600282 | 116.417432 |
| HLA-F | 701.849517 | 1393.28257 | 674.447854 | 1393.76354 | 48.7012438 | 53.3318695 |
| CIB1 | 4763.58101 | 3166.85186 | 4758.56187 | 3202.68158 | 147.70499 | 71.4313896 |
| GLIS2 | 1222.70902 | 2114.78198 | 1225.56079 | 2063.17881 | 33.6429694 | 89.4892871 |
| ANXA3 | 3226.94946 | 5045.96647 | 3191.7063 | 5122.95876 | 118.675354 | 237.78285 |
| PLBD2 | 4067.44631 | 5948.87705 | 4089.0158 | 5984.88207 | 56.593788 | 201.992686 |
| CHP1 | 8972.08893 | 12888.5708 | 9006.89591 | 12865.4819 | 117.287705 | 612.789648 |
| FADS3 | 1114.80695 | 1930.19752 | 1106.01759 | 1915.1039 | 27.2676121 | 56.6780147 |
| LAMC2 | 5033.18705 | 3373.02693 | 5017.8988 | 3422.76386 | 32.9113184 | 137.298491 |
| NAV3 | 1543.19056 | 833.677977 | 1565.0551 | 839.929074 | 45.3804457 | 23.3460461 |
| PARD3B | 497.227482 | 1056.57392 | 508.846765 | 1041.74729 | 30.2681036 | 36.5521353 |
| EPHX1 | 1983.07998 | 3293.14918 | 1946.70759 | 3217.4531 | 66.1098105 | 197.983835 |
| IQCD | 879.853082 | 386.934549 | 869.22045 | 383.74223 | 23.0711296 | 17.7038608 |
| COL4A2 | 235.08865 | 645.514369 | 240.022059 | 666.939923 | 11.1990473 | 53.481005 |
| TPP1 | 3793.78678 | 5722.0034 | 3803.02886 | 5804.08553 | 29.2610347 | 288.132501 |
| RRP1B | 4152.22104 | 6190.64092 | 4074.61448 | 6180.14233 | 206.78691 | 20.5924716 |
| HES4 | 245.484834 | 646.753373 | 250.749155 | 652.849643 | 18.850927 | 20.2597797 |
| NR4A2 | 3992.63514 | 2410.42719 | 3993.52046 | 2427.83927 | 219.715955 | 128.372837 |
| ZMYND8 | 4339.60961 | 2950.06912 | 4349.92132 | 2959.89766 | 35.3646334 | 49.7630223 |
| VWA5A | 2307.08326 | 1367.20159 | 2322.83132 | 1394.71617 | 57.5272228 | 58.3248632 |
| HYAL3 | 855.137799 | 1596.40317 | 856.398707 | 1592.53025 | 42.9880978 | 66.1068135 |
| MIR100HG | 693.671852 | 267.282584 | 680.327166 | 270.876868 | 33.3119811 | 16.8472179 |
| ADAM15 | 5766.06056 | 7947.313 | 5765.28678 | 8015.69798 | 34.727139 | 137.005488 |
| C6orf89 | 3070.83834 | 4553.87441 | 3077.40702 | 4598.43088 | 53.0387132 | 133.297064 |
| TSPAN14 | 7015.33797 | 4933.36039 | 7018.6878 | 4849.82459 | 102.551992 | 153.115185 |
| SLC6A6 | 7265.00365 | 4763.64116 | 7310.60134 | 4872.2085 | 308.546304 | 231.61506 |
| WDR1 | 7989.04276 | 11120.8584 | 7862.63825 | 11081.2783 | 279.608674 | 130.166278 |
| ITGA10 | 988.042876 | 131.686877 | 980.643015 | 135.031851 | 154.735311 | 24.0957154 |
| PTPN21 | 790.33287 | 1445.7396 | 784.392089 | 1443.27296 | 26.019187 | 10.4182261 |
| PDLIM7 | 1698.53199 | 2902.66768 | 1706.36071 | 2954.73204 | 25.8069018 | 219.429244 |
| RAPGEF1 | 3412.68027 | 4985.62635 | 3435.65219 | 5010.64699 | 86.4107476 | 101.395684 |
| CYB5B | 13194.0341 | 9931.98603 | 13203.2906 | 9904.55108 | 118.64832 | 155.332576 |
| EFEMP1 | 8111.10995 | 11079.4436 | 8049.37978 | 11087.8931 | 217.019789 | 140.834293 |
| PPP1R9B | 3274.69121 | 2162.28253 | 3292.8026 | 2170.4009 | 45.6103173 | 17.9361764 |
| NTN4 | 13432.3028 | 17809.6858 | 13442.3644 | 17818.274 | 158.216693 | 400.493373 |
| ERG28 | 5184.34758 | 7655.16523 | 5130.53934 | 7562.46103 | 103.374581 | 404.91017 |
| OLMALINC | 1054.14873 | 506.144606 | 1027.29441 | 517.817792 | 49.1513785 | 21.4096809 |
| ASAP1 | 8676.26543 | 6281.31019 | 8634.32363 | 6298.35519 | 199.942065 | 104.02921 |
| RICTOR | 2269.37527 | 1378.73564 | 2268.40515 | 1371.45393 | 37.2411815 | 50.9569431 |
| GALNS | 2486.7248 | 3737.31087 | 2456.42616 | 3768.83304 | 75.8458038 | 58.9932141 |
| VWA7 | 848.37064 | 1616.47402 | 817.364495 | 1601.59517 | 71.6720027 | 66.5030744 |
| ELOVL5 | 9467.55781 | 12938.8303 | 9507.08619 | 12887.1774 | 187.061217 | 375.318372 |
| SLC66A3 | 902.560865 | 1621.13452 | 889.079617 | 1613.33707 | 26.6030544 | 76.8178681 |
| ANTXR2 | 1191.36949 | 571.734558 | 1186.96712 | 589.913285 | 57.550727 | 51.5696179 |
| FURIN | 7456.42729 | 5373.16226 | 7440.31319 | 5417.71268 | 70.7186457 | 136.432991 |
| TCP11L1 | 819.017587 | 1503.38746 | 831.793559 | 1513.52533 | 44.0509863 | 46.5762941 |
| COL4A1 | 131.048099 | 472.946395 | 128.320769 | 471.777212 | 12.6236657 | 71.9651289 |
| GPR155 | 824.45564 | 1532.59508 | 841.997384 | 1551.75088 | 56.2522749 | 46.4179864 |
| ABTB2 | 721.745372 | 1400.50211 | 698.944236 | 1416.07315 | 56.3891982 | 50.3302642 |
| ZFYVE19 | 3097.54611 | 1765.62827 | 3248.07627 | 1773.87235 | 271.520814 | 32.7129683 |
| TBC1D2 | 524.425196 | 1040.41262 | 523.212661 | 1028.9906 | 5.72092484 | 25.5555 |
| PHLDA1 | 1388.63823 | 680.206036 | 1422.85077 | 689.249533 | 96.7898143 | 53.2466272 |
| ZMIZ2 | 2207.24629 | 3325.31609 | 2187.56359 | 3337.42874 | 39.6212949 | 72.2161382 |
| LDHA | 109777.896 | 85381.6044 | 109352.873 | 85865.9595 | 1386.02999 | 2010.91967 |
| CEACAM1 | 208.402688 | 12.2000344 | 200.210566 | 12.3974771 | 18.3862475 | 0.83252578 |
| CYB5R3 | 6758.86548 | 9324.82052 | 6778.08636 | 9482.94768 | 100.11731 | 288.396613 |
| GLI2 | 261.331814 | 649.579204 | 263.064177 | 662.231902 | 10.7931526 | 37.8188251 |
| SLC20A1 | 6236.21642 | 4363.59274 | 6137.52293 | 4400.86414 | 191.816314 | 95.1642764 |
| CTH | 2022.71235 | 1162.86278 | 2016.1853 | 1149.53202 | 71.5990643 | 71.1622538 |
| POMP | 2801.02237 | 1810.7904 | 2806.83551 | 1818.26098 | 49.6391255 | 35.837825 |
| TSC22D1 | 6999.1547 | 4917.59943 | 7051.8481 | 4912.5003 | 178.502681 | 161.805619 |
| SPTAN1 | 14587.358 | 19039.6222 | 14507.8933 | 19086.4585 | 170.991565 | 355.111869 |
| FGB | 51212.5987 | 66104.6487 | 51439.537 | 66489.6832 | 1199.3938 | 987.877383 |
| IGSF9 | 512.235844 | 1050.24813 | 504.413999 | 1054.42262 | 35.5943926 | 46.058573 |
| NXPH3 | 64.1521459 | 286.524826 | 64.1451328 | 278.283031 | 3.05053017 | 15.826485 |
| PLXNA1 | 2660.0071 | 3946.0655 | 2646.61587 | 3997.69111 | 50.7302753 | 126.396846 |
| STC2 | 6982.9448 | 4747.23335 | 7064.71167 | 4776.12805 | 219.725721 | 234.628127 |
| HK2 | 2866.64702 | 1640.45993 | 2957.48821 | 1658.16256 | 199.105369 | 112.085982 |
| SLAIN1 | 1282.39496 | 697.287151 | 1285.81834 | 692.99332 | 13.8370176 | 14.9435165 |
| RHOA | 19898.9868 | 26133.6155 | 19721.7126 | 26157.6762 | 560.549808 | 280.851242 |
| PRKACB | 2683.21845 | 1704.98312 | 2661.05111 | 1699.48749 | 96.3157655 | 16.4276758 |
| ZNFX1 | 2684.91348 | 4161.20091 | 2687.86183 | 4106.66429 | 76.8567951 | 241.712151 |
| SGPL1 | 4408.38053 | 6229.43008 | 4421.20633 | 6250.2134 | 46.3098799 | 210.856178 |
| MYO1C | 18368.4088 | 24180.0545 | 18256.0778 | 24180.0948 | 529.323206 | 275.632234 |
| PKM | 106907.605 | 84847.2489 | 106797.335 | 84503.2702 | 1407.39297 | 1250.45177 |
| C1QL1 | 503.021216 | 1025.59958 | 502.470207 | 1043.85491 | 6.94750864 | 69.1068966 |
| IFNAR1 | 3195.27988 | 4534.40113 | 3204.34095 | 4557.03141 | 19.5129492 | 59.1999231 |
| STN1 | 1493.48928 | 772.810359 | 1489.4375 | 752.113611 | 35.3953177 | 85.2979534 |
| APOE | 964.506955 | 2704.4654 | 902.891339 | 2809.83668 | 110.10052 | 190.844175 |
| TYMP | 878.177874 | 1585.41559 | 896.088067 | 1565.44257 | 31.5723169 | 96.3525761 |
| EPHA10 | 384.943677 | 821.59648 | 384.995383 | 828.431754 | 10.7379757 | 28.5689625 |
| SLC41A2 | 436.811775 | 891.94745 | 439.19311 | 890.507703 | 4.58883088 | 19.7177729 |
| SOWAHD | 33.4641326 | 219.104418 | 33.6030883 | 213.85648 | 7.42595033 | 9.56199507 |
| FOCAD | 5043.25527 | 3210.35922 | 5002.05971 | 3083.48168 | 175.003918 | 241.20185 |
| AKAP5 | 471.459113 | 949.533306 | 465.926603 | 949.919714 | 10.7821507 | 36.2608711 |
| SSTR1 | 378.82275 | 103.477169 | 386.915559 | 103.836133 | 30.3526123 | 8.25621773 |
| SEC61A1 | 11469.6267 | 15373.5214 | 11520.0988 | 15363.5735 | 269.215505 | 396.840834 |
| PLSCR1 | 1280.49369 | 699.403549 | 1288.73403 | 704.279857 | 14.8518599 | 24.5209041 |
| TLE1 | 1176.67684 | 1936.60355 | 1179.32898 | 1923.22576 | 43.4674699 | 28.3185128 |
| MAFF | 931.931617 | 417.108748 | 926.216841 | 405.186648 | 14.0077342 | 54.1925889 |
| PPIL3 | 1198.54912 | 644.118002 | 1186.2033 | 645.701932 | 30.5462223 | 3.65931384 |
| TRPC4 | 605.487547 | 1143.28621 | 610.616119 | 1134.36915 | 13.9829086 | 41.2645823 |
| PVR | 3660.74981 | 5252.71323 | 3627.21336 | 5276.45566 | 143.753155 | 41.4302451 |
| OBSL1 | 931.917223 | 1631.72396 | 963.148888 | 1627.51851 | 58.9933898 | 8.509695 |
| SOWAHC | 2496.45487 | 1525.47755 | 2529.75231 | 1517.65782 | 114.944381 | 71.3654587 |
| BLVRB | 4236.64428 | 5895.01991 | 4260.79185 | 5844.16843 | 88.725404 | 106.04037 |
| RAB15 | 1519.19914 | 2376.51567 | 1515.40718 | 2375.81586 | 46.8442154 | 7.93749633 |
| ATP11A | 9460.95803 | 7030.28524 | 9473.19063 | 7017.96819 | 152.717631 | 151.369889 |
| AADACP1 | 261.438131 | 51.1985119 | 264.984353 | 51.9180664 | 8.20296126 | 4.39262149 |
| MAP3K5 | 2227.17673 | 1408.16826 | 2217.35234 | 1396.11192 | 39.4482032 | 24.8768161 |
| WFS1 | 2341.7955 | 3477.64066 | 2338.03497 | 3493.18294 | 35.5722764 | 114.826111 |
| CREG1 | 4673.30246 | 6401.27005 | 4672.87573 | 6473.3095 | 13.2526739 | 138.627801 |
| IGFBP1 | 202.180888 | 7.72023531 | 190.953526 | 8.21933004 | 21.0387946 | 1.87784053 |
| HNRNPA2B1 | 37794.6505 | 28297.1599 | 38498.7424 | 28103.0143 | 1477.82778 | 478.989619 |
| CTXN1 | 303.763656 | 739.961906 | 310.1085 | 738.139465 | 28.4453593 | 68.0805507 |
| AADAC | 306.591081 | 60.0855865 | 309.148412 | 59.8836903 | 28.1351988 | 17.2985809 |
| LRRC8A | 1328.2894 | 2165.60825 | 1356.60468 | 2162.32663 | 63.3782745 | 56.7380244 |
| TTC9 | 623.59764 | 1177.24047 | 625.017442 | 1144.70039 | 16.470626 | 62.5369509 |
| ITGB2 | 1510.18424 | 866.426674 | 1494.02039 | 852.461944 | 45.7640954 | 24.4726308 |
| TNNC1 | 12.4495426 | 163.853844 | 12.2210257 | 168.169389 | 1.06135591 | 7.67450579 |
| TRAF3 | 3467.77901 | 5066.93215 | 3422.04564 | 5036.05244 | 148.20809 | 141.580685 |
| OS9 | 5142.62615 | 6923.28515 | 5147.16096 | 6903.97417 | 12.2158816 | 86.6929002 |
| VCL | 15308.7031 | 20157.0285 | 15089.7068 | 20104.4813 | 468.732688 | 258.396413 |
| TNESF10 | 889.038509 | 428.888357 | 872.762564 | 417.601838 | 29.9522995 | 22.2989396 |
| CLIC1 | 22361.5226 | 17285.8872 | 22423.5834 | 17291.122 | 554.461747 | 113.887359 |
| CDR2L | 1033.25108 | 1712.63919 | 1033.1254 | 1713.14322 | 10.8205015 | 44.9286258 |
| DIO3 | 308.513777 | 714.574334 | 305.175329 | 731.520373 | 26.9634224 | 38.5812733 |
| TENT5B | 513.48388 | 1028.45673 | 535.729236 | 1030.02372 | 45.0388379 | 7.42715497 |
| TPM4 | 38613.4999 | 49844.1903 | 38555.1124 | 49468.6249 | 1085.22043 | 710.923543 |
| CRLF2 | 240.233653 | 41.1087741 | 239.837629 | 41.3249237 | 14.9557382 | 7.14354388 |
| CCDC102A | 766.519187 | 1382.74948 | 743.500403 | 1401.94804 | 49.0049605 | 35.3368964 |
| GPX1 | 6287.91638 | 4314.91437 | 6235.68442 | 4340.80181 | 257.928711 | 174.96091 |
| MIDN | 4820.85218 | 3383.59476 | 4798.2802 | 3402.89065 | 90.4515896 | 102.845955 |
| CELSR2 | 1794.4882 | 2883.71996 | 1778.56959 | 2806.31411 | 72.9590307 | 171.955015 |

| row | t.test | t.test.adj | baseMean | log2FoldChange | pvalue | padj |
|---|---|---|---|---|---|---|
| | Combined differentially expressed genes (DEG) for H2122 | | | | | |
| | and H2030; data used to generate the heatmap for FIG. 3B. | | | | | |
| SAT1 | 1.71E−11 | 6.28E−07 | 1966.64678 | −1.2945604 | 2.14E−214 | 4.33E−210 |
| ITGA2 | 2.39E−06 | 0.08770937 | 3741.77613 | −1.4686139 | 9.96E−181 | 1.01E−176 |
| NT5E | 8.01E−05 | 1 | 7100.71597 | −2.5716242 | 4.22E−94 | 2.84E−90 |
| PSME1 | 1.67E−07 | 0.00612364 | 4647.85356 | 0.52276556 | 6.50E−81 | 3.28E−77 |
| GRAMD1B | 2.91E−10 | 1.07E−05 | 17759.6149 | −1.4768024 | 1.04E−68 | 4.21E−65 |
| TOR4A | 3.58E−09 | 0.00013163 | 1852.05636 | −0.7432352 | 1.52E−59 | 5.12E−56 |
| JDP2 | 2.52E−08 | 0.00092405 | 426.032363 | 1.05957827 | 1.57E−58 | 4.52E−55 |
| DUSP8 | 1.19E−05 | 0.43506268 | 301.433328 | 1.18122313 | 2.49E−51 | 5.58E−48 |
| UBALD2 | 2.25E−06 | 0.08259294 | 477.187026 | −1.2637123 | 2.38E−51 | 5.58E−48 |
| ETV4 | 0.00035901 | 1 | 680.070085 | −4.1892067 | 1.66E−46 | 3.36E−43 |
| SPTAN1 | 2.51E−08 | 0.00092121 | 15726.0496 | 0.38808919 | 1.08E−40 | 1.97E−37 |
| TPRA1 | 8.36E−08 | 0.00306988 | 1724.96243 | 0.72986266 | 2.71E−40 | 4.56E−37 |
| UPP1 | 6.23E−05 | 1 | 1849.15154 | −1.0414443 | 6.15E−40 | 9.55E−37 |
| ARPC5 | 2.49E−08 | 0.0009145 | 6432.37745 | 0.39594904 | 7.18E−39 | 1.04E−35 |
| HLA-C | 6.40E−08 | 0.00235034 | 9903.96188 | 0.66774943 | 1.16E−38 | 1.56E−35 |
| DAPK1 | 5.26E−07 | 0.0192934 | 2020.78971 | 1.05486077 | 1.12E−37 | 1.41E−34 |
| AC004585.1 | 9.75E−07 | 0.03578428 | 51.1460768 | −2.7941875 | 6.41E−37 | 7.62E−34 |
| VPS39 | 6.59E−08 | 0.00241849 | 3030.2371 | 0.39956653 | 1.13E−34 | 1.26E−31 |
| ERV3-1 | 1.06E−05 | 0.38972855 | 300.927716 | 1.02128659 | 7.44E−34 | 7.90E−31 |
| UBLCP1 | 1.84E−06 | 0.06745042 | 2109.16894 | −0.4049912 | 3.35E−32 | 3.38E−29 |
| ARHGEF17 | 6.65E−05 | 1 | 1825.10372 | 0.70748681 | 5.22E−30 | 5.02E−27 |
| SMAP2 | 4.31E−07 | 0.01583134 | 1202.12996 | −0.4438382 | 2.97E−29 | 2.73E−26 |
| FLNB | 1.09E−07 | 0.00398443 | 27911.4459 | −0.2936348 | 3.67E−29 | 3.22E−26 |
| IL4R | 0.00016361 | 1 | 1093.46042 | −0.8546016 | 2.19E−28 | 1.84E−25 |
| KCTD15 | 6.82E−07 | 0.02501543 | 1360.4034 | 0.68029533 | 1.57E−27 | 1.27E−24 |
| SPRY2 | 0.0003467 | 1 | 484.87247 | −1.8605493 | 2.52E−27 | 1.96E−24 |
| SPRED1 | 4.79E−07 | 0.01756453 | 1172.96649 | −1.3763602 | 2.81E−27 | 2.10E−24 |
| TMEM54 | 2.49E−08 | 0.00091367 | 1106.40396 | 0.38521747 | 1.12E−26 | 8.08E−24 |
| GLB1 | 5.13E−07 | 0.01881908 | 2151.87612 | 0.54688353 | 1.22E−26 | 8.48E−24 |
| DUSP5 | 1.28E−07 | 0.00469965 | 362.473906 | −0.9317928 | 3.05E−26 | 2.05E−23 |
| SLC6A9 | 8.81E−06 | 0.3227965 | 728.25928 | 0.74078103 | 6.84E−26 | 4.46E−23 |
| MANBA | 2.29E−05 | 0.83879508 | 884.003309 | 0.54444323 | 2.11E−25 | 1.33E−22 |
| CELSR1 | 1.18E−05 | 0.43175292 | 3951.34689 | 0.5268582 | 2.32E−25 | 1.42E−22 |
| PTPN12 | 4.62E−07 | 0.01694137 | 5896.6553 | −0.6934759 | 2.771−25 | 1.65E−22 |
| IRF1 | 3.88E−07 | 0.01422651 | 555.476904 | 0.59168701 | 3.02E−25 | 1.74E−22 |
| TXNRD1 | 2.33E−06 | 0.08534909 | 67513.7262 | 0.27236549 | 3.58E−25 | 2.01E−22 |
| MAN1B1 | 2.25E−07 | 0.00826735 | 2771.52525 | 0.31884998 | 4.55E−25 | 2.48E−22 |
| HMMR | 3.65E−06 | 0.13379633 | 2727.58325 | −0.4699317 | 9.51E−25 | 5.06E−22 |
| PTPN3 | 2.10E−07 | 0.00771034 | 1858.85227 | −0.3598073 | 1.35E−24 | 7.00E−22 |
| TCTN1 | 2.76E−08 | 0.001013 | 669.592913 | 0.42886132 | 2.20E−24 | 1.11E−21 |
| HIF1A | 2.16E−06 | 0.079198 | 8993.82042 | −0.441083 | 4.41E−24 | 2.17E−21 |
| APOL2 | 1.15E−06 | 0.0420559 | 1391.85358 | 0.59483761 | 1.33E−23 | 6.40E−21 |
| PACS2 | 3.32E−05 | 1 | 2647.42303 | 0.46505063 | 1.01E−22 | 4.74E−20 |
| MYORG | 4.64E−06 | 0.17015956 | 2038.83441 | 0.62852083 | 1.51E−22 | 6.95E−20 |
| SRPK1 | 5.60E−07 | 0.0205448 | 3513.16642 | −0.3988424 | 7.89E−22 | 3.54E−19 |
| AC090409.1 | 3.00E−09 | 0.00010999 | 28.5044565 | −2.3031224 | 9.37E−22 | 4.111−19 |
| DUSP7 | 5.53E−07 | 0.02030585 | 904.896158 | −0.5850978 | 1.01E−21 | 4.33E−19 |
| PLEKHA8 | 4.82E−08 | 0.00176928 | 710.602641 | −0.4048962 | 2.06E−21 | 8.67E−19 |
| KBTBD2 | 9.23E−06 | 0.33837241 | 1707.28603 | −0.4883648 | 5.17E−21 | 2.13E−18 |
| APMAP | 1.19E−06 | 0.04376718 | 3788.41555 | 0.24993726 | 7.52E−21 | 3.04E−18 |
| TAGLN2 | 1.03E−05 | 0.37773542 | 47165.8285 | 0.29038065 | 1.66E−20 | 6.57E−18 |
| LRCH1 | 1.10E−05 | 0.40439475 | 652.347919 | −0.4536442 | 1.99E−20 | 7.71E−18 |
| ELMO3 | 3.60E−07 | 0.01323215 | 1854.68384 | 0.37245357 | 2.96E−20 | 1.11E−17 |
| HIBADH | 5.52E−07 | 0.02025373 | 3175.66743 | 0.26469635 | 2.97E−20 | 1.11E−17 |
| PHKB | 2.77E−07 | 0.01017073 | 4801.46776 | 0.21644172 | 8.07E−20 | 2.96E−17 |
| TCF7L2 | 3.70E−07 | 0.01357654 | 1408.74657 | −0.356114 | 1.57E−19 | 5.67E−17 |
| LUCAT1 | 1.29E−06 | 0.04734185 | 1420.17229 | −0.946835 | 2.49E−19 | 8.84E−17 |
| RHOA | 4.80E−05 | 1 | 20683.7772 | 0.32880921 | 1.70E−18 | 5.93E−16 |
| MLX | 1.34E−05 | 0.49027013 | 2793.60685 | −0.7607038 | 2.47E−18 | 8.45E−16 |
| PPP1R13L | 6.87E−06 | 0.25188359 | 2324.24463 | 0.78203397 | 3.00E−18 | 1.01E−15 |
| RGS2 | 0.00084199 | 1 | 1176.17397 | −1.7698234 | 5.50E−18 | 1.82E−15 |
| DAAM1 | 7.92E−07 | 0.02906946 | 1154.51653 | 0.36350161 | 5.74E−18 | 1.87E−15 |
| MYL6 | 0.00045748 | 1 | 14540.3159 | 0.51912317 | 7.91E−18 | 2.53E−15 |
| CRYAB | 0.00141089 | 1 | 24.9010519 | 3.50819543 | 1.06E−17 | 3.30E−15 |
| DPY19L1 | 5.76E−06 | 0.21135878 | 4249.52314 | −0.372659 | 1.05E−17 | 3.30E−15 |
| UBE2Q2 | 3.47E−05 | 1 | 2617.50379 | 0.29218612 | 1.44E−17 | 4.40E−15 |
| DDR1 | 0.00031189 | 1 | 4828.69031 | 0.39795539 | 2.23E−17 | 6.73E−15 |
| MXD4 | 0.00024025 | 1 | 860.676442 | 0.74021552 | 4.96E−17 | 1.47E−14 |
| APH1A | 1.36E−05 | 0.49756325 | 4902.18379 | 0.26162576 | 7.01E−17 | 2.05E−14 |
| TRAM1 | 6.81E−05 | 1 | 7627.38422 | 0.87845765 | 1.73E−16 | 4.98E−14 |
| NIPSNAP3A | 2.90E−06 | 0.10651608 | 492.104506 | 0.56334712 | 1.99E−16 | 5.66E−14 |
| KIF14 | 2.24E−06 | 0.08223749 | 1953.59614 | −0.383236 | 2.13E−16 | 5.98E−14 |
| SCAT8 | 2.77E−06 | 0.10153471 | 48.3069007 | −1.4095213 | 3.26E−16 | 9.02E−14 |
| MRGBP | 6.82E−06 | 0.24990413 | 1976.81376 | −0.4682909 | 3.60E−16 | 9.83E−14 |
| SPRY4-AS1 | 2.62E−06 | 0.0960672 | 33.2789516 | −2.0207625 | 6.20E−16 | 1.67E−13 |
| RCN1 | 1.96E−06 | 0.07206964 | 1701.07179 | 0.29115341 | 9.52E−16 | 2.53E−13 |
| TMEM14A | 1.66E−08 | 0.00060943 | 1221.95573 | 0.24769242 | 1.11E−15 | 2.92E−13 |
| GPX1 | 4.70E−06 | 0.17251319 | 4869.50465 | −0.4285291 | 1.35E−15 | 3.49E−13 |
| BCAR3 | 2.02E−05 | 0.73888104 | 2574.49674 | −0.498745 | 1.72E−15 | 4.40E−13 |
| STX7 | 2.91E−06 | 0.10672672 | 1142.07419 | 0.33698731 | 2.15E−15 | 5.44E−13 |
| LRSAM1 | 7.62E−06 | 0.27938275 | 959.18585 | 0.32924287 | 2.44E−15 | 6.08E−13 |
| EPHA2 | 0.00133399 | 1 | 4147.51425 | −0.8885599 | 3.66E−15 | 9.02E−13 |
| CASC8 | 3.93E−06 | 0.14421162 | 61.688145 | −1.5499087 | 3.77E−15 | 9.17E−13 |
| SERPIND1 | 0.00053705 | 1 | 20.7410828 | −3.8492298 | 4.01E−15 | 9.63E−13 |
| CTSL | 1.62E−05 | 0.5947699 | 12950.6094 | −1.1410187 | 6.07E−15 | 1.44E−12 |
| STAMBPL1 | 0.00153792 | 1 | 505.992343 | −1.2802265 | 6.39E−15 | 1.50E−12 |
| ANKRD2 | 0.00147759 | 1 | 274.915477 | 1.14063448 | 8.38E−15 | 1.95E−12 |
| POLE2 | 1.41E−05 | 0.51568513 | 709.734681 | −0.4603422 | 1.56E−14 | 3.57E−12 |
| UTP11 | 4.24E−06 | 0.15549793 | 1853.41543 | −0.329192 | 3.21E−14 | 7.27E−12 |
| SMAD6 | 0.00015322 | 1 | 1251.11673 | 0.50379865 | 3.41E−14 | 7.64E−12 |
| TINAG | 0.00159098 | 1 | 33.0099058 | −2.5396201 | 3.72E−14 | 8.24E−12 |
| AC012313.1 | 4.55E−06 | 0.16672 | 264.142291 | −0.5766278 | 4.91E−14 | 1.08E−11 |
| PIGT | 0.00023417 | 1 | 5899.59314 | 0.31336608 | 6.63E−14 | 1.44E−11 |
| ADSSL1 | 2.34E−05 | 0.85643624 | 224.919355 | 0.73151325 | 8.18E−14 | 1.76E−11 |
| PPIC | 6.44E−06 | 0.23594469 | 781.647557 | 0.41568427 | 9.27E−14 | 1.97E−11 |
| DNAJB11 | 8.13E−06 | 0.29790123 | 1315.71177 | −0.2718711 | 1.34E−13 | 2.83E−11 |
| ERBB2 | 6.94E−05 | 1 | 5048.03979 | 0.68276748 | 1.43E−13 | 2.94E−11 |
| PTTG1 | 6.90E−06 | 0.25300016 | 3397.34965 | −0.3516834 | 1.41E−13 | 2.94E−11 |
| RIPK4 | 0.00140969 | 1 | 1250.67328 | −0.7665708 | 1.61E−13 | 3.28E−11 |
| SLCO2B1 | 0.0001863 | 1 | 26.0573096 | 2.02924758 | 2.97E−13 | 5.99E−11 |
| HSD17B4 | 2.18E−05 | 0.79810989 | 3588.70717 | 0.229865 | 6.14E−13 | 1.23E−10 |
| GRHPR | 2.86E−05 | 1 | 2519.98144 | 0.26459733 | 6.92E−13 | 1.37E−10 |
| DGCR2 | 0.00019244 | 1 | 2822.88095 | 0.48142917 | 8.00E−13 | 1.57E−10 |
| HMGA1 | 0.00107795 | 1 | 17885.4431 | −0.7467857 | 9.09E−13 | 1.76E−10 |
| FARP2 | 7.56E−06 | 0.27721063 | 1264.51138 | 0.3205733 | 1.26E−12 | 2.43E−10 |
| MRPS18B | 6.74E−06 | 0.247281 | 2751.25705 | −0.2527737 | 1.70E−12 | 3.24E−10 |
| AC102953.2 | 1.15E−05 | 0.42008672 | 464.588282 | 0.54511672 | 1.82E−12 | 3.43E−10 |
| SLC16A14 | 1.21E−05 | 0.44205103 | 2374.55321 | −0.6585766 | 1.83E−12 | 3.43E−10 |
| UPK3B | 0.01904601 | 1 | 183.870806 | 3.05524247 | 1.97E−12 | 3.64E−10 |
| SAMD11 | 0.00315091 | 1 | 267.055276 | 1.72096946 | 2.29E−12 | 4.21E−10 |
| P4HTM | 9.80E−06 | 0.35928603 | 1433.95098 | 0.43527921 | 2.36E−12 | 4.30E−10 |
| OAF | 9.74E−06 | 0.35690283 | 556.833648 | −0.4738616 | 3.03E−12 | 5.46E−10 |
| RHOB | 0.00268768 | 1 | 4303.30197 | 0.93794423 | 3.62E−12 | 6.46E−10 |
| TINCR | 0.00019562 | 1 | 164.563293 | 0.85314119 | 4.46E−12 | 7.91E−10 |
| ABALON | 1.84E−05 | 0.67279504 | 78.2646672 | −0.8630458 | 4.72E−12 | 8.29E−10 |
| RBMS2 | 5.07E−05 | 1 | 4059.39257 | −0.3543295 | 5.09E−12 | 8.86E−10 |
| SPATS2L | 1.76E−05 | 0.64485311 | 2574.61819 | 0.35300467 | 5.79E−12 | 1.00E−09 |
| AMBRA1 | 3.21E−05 | 1 | 883.340476 | 0.28909505 | 7.57E−12 | 1.30E−09 |
| RPRD1B | 3.01E−06 | 0.11026809 | 1361.22635 | −0.243231 | 8.55E−12 | 1.45E−09 |
| FOSL2 | 8.54E−05 | 1 | 9011.69714 | 0.56962042 | 9.90E−12 | 1.67E−09 |
| RRM2 | 4.39E−05 | 1 | 17817.4507 | −0.2773851 | 1.07E−11 | 1.79E−09 |
| FOSL1 | 0.00337077 | 1 | 1480.593 | −1.3686494 | 1.09E−11 | 1.80E−09 |
| CDK17 | 1.03E−05 | 0.37913261 | 882.269417 | −0.3581992 | 1.20E−11 | 1.97E−09 |
| ACKR3 | 0.00016161 | 1 | 17.1618886 | 2.38728015 | 1.45E−11 | 2.34E−09 |
| ATP6AP1L | 2.30E−06 | 0.08435576 | 57.518215 | −1.0262492 | 1.44E−11 | 2.34E−09 |
| TTC14 | 0.00013074 | 1 | 436.585437 | 0.59117959 | 1.87E−11 | 3.00E−09 |
| TCTN3 | 2.07E−05 | 0.75808712 | 3096.83555 | 0.38605016 | 2.11E−11 | 3.36E−09 |
| RGP1 | 0.00081313 | 1 | 1891.98126 | −0.5770791 | 2.44E−11 | 3.85E−09 |
| RPF1 | 1.53E−05 | 0.56063713 | 1355.48529 | −0.2834688 | 2.67E−11 | 4.17E−09 |
| MR1 | 1.82E−05 | 0.66633758 | 684.577291 | 0.57614483 | 2.83E−11 | 4.40E−09 |
| THUMPD3 | 6.53E−05 | 1 | 1504.75697 | −0.2614922 | 3.61E−11 | 5.57E−09 |
| C5orf51 | 1.80E−05 | 0.66092548 | 2438.90076 | −0.3002296 | 3.68E−11 | 5.63E−09 |
| CHMP7 | 1.09E−05 | 0.40065396 | 2098.5038 | −0.2102858 | 3.99E−11 | 6.05E−09 |
| NHS | 2.23E−05 | 0.81505166 | 762.113745 | −0.5031508 | 4.02E−11 | 6.05E−09 |
| PDXK | 0.00336853 | 1 | 10147.1151 | 1.02060271 | 6.00E−11 | 8.97E−09 |
| FKBP9 | 0.0010492 | 1 | 6382.71569 | 0.36415188 | 7.13E−11 | 1.06E−08 |
| EBNA1BP2 | 2.78E−05 | 1 | 4741.46241 | −0.2523832 | 7.44E−11 | 1.10E−08 |
| GAS2L3 | 1.50E−05 | 0.54869094 | 843.882388 | −0.3457072 | 7.63E−11 | 1.12E−08 |
| PRR11 | 0.00031312 | 1 | 2864.44929 | −0.2838038 | 9.34E−11 | 1.36E−08 |
| EIF2B2 | 0.00066243 | 1 | 1224.71095 | −0.3883521 | 1.04E−10 | 1.48E−08 |
| SIL1 | 2.48E−05 | 0.90965892 | 1013.04277 | 0.28861576 | 1.03E−10 | 1.48E−08 |
| CHD3 | 0.00025645 | 1 | 4366.40235 | 0.35298789 | 1.20E−10 | 1.70E−08 |
| RAB27A | 0.00018757 | 1 | 1544.39054 | −0.3174935 | 1.87E−10 | 2.65E−08 |
| TMEM94 | 0.00012362 | 1 | 1764.21933 | 0.46060096 | 2.07E−10 | 2.91E−08 |
| PTPN13 | 1.76E−05 | 0.64596459 | 673.593974 | 0.39648826 | 2.16E−10 | 3.01E−08 |
| PAIP2 | 2.39E−05 | 0.8752369 | 977.086199 | −0.3684181 | 2.31E−10 | 3.19E−08 |
| TRAPPC6A | 0.00201787 | 1 | 328.735325 | 0.91869453 | 3.61E−10 | 4.96E−08 |
| CDK5R1 | 3.57E−05 | 1 | 242.526119 | −0.6715964 | 5.02E−10 | 6.85E−08 |
| SGPL1 | 4.64E−05 | 1 | 4929.89815 | 0.35952276 | 5.65E−10 | 7.65E−08 |
| SUMF1 | 0.00013391 | 1 | 1596.09712 | 0.52706294 | 5.71E−10 | 7.68E−08 |
| CHP1 | 0.00013766 | 1 | 10956.4193 | 0.5271303 | 6.48E−10 | 8.66E−08 |
| TM2D2 | 0.00016057 | 1 | 1196.75911 | 0.46656605 | 6.76E−10 | 8.98E−08 |
| TUFT1 | 0.00474432 | 1 | 3458.86391 | 0.98451593 | 6.85E−10 | 9.04E−08 |
| EIF3G | 2.03E−05 | 0.74294034 | 3839.96282 | −0.1471823 | 6.92E−10 | 9.07E−08 |
| CCT5 | 6.59E−05 | 1 | 31609.2626 | −0.1804067 | 7.28E−10 | 9.48E−08 |
| RNPEPL1 | 0.00077398 | 1 | 2317.23092 | 0.31716685 | 7.40E−10 | 9.57E−08 |
| EVI2B | 5.19E−05 | 1 | 5.77544282 | −5.3879134 | 7.55E−10 | 9.71E−08 |
| AREG | 0.00515204 | 1 | 2486.33136 | −0.9670701 | 8.38E−10 | 1.07E−07 |
| BMF | 0.01844838 | 1 | 351.116551 | 2.17817603 | 8.64E−10 | 1.10E−07 |
| COMMD6 | 0.00048821 | 1 | 692.431143 | 0.39130139 | 9.39E−10 | 1.18E−07 |
| CUL3 | 3.88E−05 | 1 | 3548.0094 | −0.143749 | 9.45E−10 | 1.19E−07 |
| CXCL8 | 0.00906443 | 1 | 653.2917 | −2.5020044 | 9.54E−10 | 1.19E−07 |
| GNG11 | 0.00374632 | 1 | 2413.79718 | −0.8822274 | 1.02E−09 | 1.26E−07 |
| SOX12 | 0.00028695 | 1 | 1286.73442 | 0.64328933 | 1.24E−09 | 1.53E−07 |
| ZNF275 | 1.96E−05 | 0.71966986 | 528.050127 | −0.3835262 | 1.32E−09 | 1.61E−07 |
| SLC25A37 | 0.00113723 | 1 | 3169.41343 | −0.3053596 | 1.34E−09 | 1.63E−07 |
| TOP1 | 0.00080243 | 1 | 5517.74315 | −0.3828122 | 1.56E−09 | 1.89E−07 |
| NAV3 | 0.0006026 | 1 | 945.79731 | −0.8616968 | 1.67E−09 | 2.01E−07 |
| PLEKHM1 | 4.98E−06 | 0.18260284 | 711.579564 | 0.27222282 | 2.16E−09 | 2.58E−07 |
| MTCL1 | 0.00145174 | 1 | 2094.44997 | 0.42573746 | 2.20E−09 | 2.61E−07 |
| MECR | 4.89E−05 | 1 | 734.698184 | 0.27954046 | 2.38E−09 | 2.80E−07 |
| WDR4 | 0.00031625 | 1 | 991.564838 | −0.4644386 | 2.42E−09 | 2.84E−07 |
| GPATCH11 | 2.71E−05 | 0.9935208 | 629.977217 | −0.377269 | 2.47E−09 | 2.88E−07 |
| SH3GLB1 | 5.59E−05 | 1 | 2784.62804 | 0.14712831 | 2.92E−09 | 3.39E−07 |
| MT-ND1 | 6.95E−05 | 1 | 53426.8211 | 0.39837347 | 2.98E−09 | 3.44E−07 |
| MYH15 | 0.00110713 | 1 | 10.8311162 | −2.4594574 | 3.14E−09 | 3.58E−07 |
| PAPSS1 | 0.00028698 | 1 | 1309.79669 | 0.27983787 | 3.13E−09 | 3.58E−07 |
| RABL2B | 1.27E−05 | 0.46611446 | 280.189861 | 0.41395283 | 3.78E−09 | 4.26E−07 |
| ZER1 | 0.00039712 | 1 | 968.012252 | 0.31313931 | 3.77E−09 | 4.26E−07 |
| PPFIBP2 | 3.05E−05 | 1 | 155.758686 | 0.82506862 | 3.94E−09 | 4.42E−07 |
| GDE1 | 0.00023082 | 1 | 2743.06589 | 0.23672162 | 4.01E−09 | 4.47E−07 |
| WBP2 | 0.00041352 | 1 | 2780.21234 | −0.3199116 | 4.57E−09 | 5.07E−07 |
| AL606500.1 | 0.00156203 | 1 | 35.6436085 | −1.7211486 | 4.89E−09 | 5.40E−07 |
| RHBDF2 | 4.11E−05 | 1 | 791.660661 | 0.31195479 | 5.07E−09 | 5.56E−07 |
| MMP24 | 0.02876002 | 1 | 188.313572 | 2.96711502 | 5.23E−09 | 5.71E−07 |
| KLF9 | 6.36E−06 | 0.23326639 | 406.023955 | 0.32888195 | 6.32E−09 | 6.86E−07 |
| DUSP6 | 1.68E−05 | 0.61650409 | 288.754067 | −3.0187732 | 7.09E−09 | 7.66E−07 |
| GPD2 | 0.00035627 | 1 | 4645.26212 | −0.273439 | 7.53E−09 | 8.09E−07 |
| SHB | 0.00123062 | 1 | 512.897928 | −0.4955481 | 8.01E−09 | 8.55E−07 |
| TNNC1 | 0.0329241 | 1 | 49.9722658 | 3.15703756 | 8.13E−09 | 8.64E−07 |
| KLHL22 | 0.00066844 | 1 | 595.022067 | 0.3767482 | 9.06E−09 | 9.58E−07 |
| FRRS1 | 0.00147229 | 1 | 255.579692 | −0.7453013 | 9.25E−09 | 9.72E−07 |
| ZC2HC1A | 5.76E−06 | 0.21126734 | 134.950223 | 0.5582365 | 1.11E−08 | 1.16E−06 |
| SEC31A | 0.00021083 | 1 | 5693.12713 | 0.31937652 | 1.24E−08 | 1.29E−06 |
| BNIP2 | 0.00021146 | 1 | 3177.84399 | −0.4991537 | 1.34E−08 | 1.39E−06 |
| PGRMC2 | 0.00113852 | 1 | 2580.63814 | 0.58475719 | 1.43E−08 | 1.47E−06 |
| TSC2 | 0.0001937 | 1 | 2402.55251 | 0.31108198 | 1.63E−08 | 1.67E−06 |
| DGUOK | 8.03E−05 | 1 | 1029.22953 | −0.3426569 | 1.68E−08 | 1.71E−06 |
| CDV3 | 0.00169079 | 1 | 9042.74091 | 0.39887878 | 1.76E−08 | 1.78E−06 |
| RTN4 | 0.00047211 | 1 | 15927.858 | 0.19722383 | 1.76E−08 | 1.78E−06 |
| PPIL3 | 0.00449706 | 1 | 766.350444 | −0.6599149 | 2.06E−08 | 2.07E−06 |
| ATP2B4 | 7.77E−05 | 1 | 2237.2065 | 0.29690461 | 2.57E−08 | 2.56E−06 |
| ABTB2 | 0.00404257 | 1 | 859.148105 | 0.77249361 | 3.15E−08 | 3.13E−06 |
| CHMP3 | 0.00013332 | 1 | 2248.44223 | 0.28358125 | 3.18E−08 | 3.14E−06 |
| RIN1 | 0.00027969 | 1 | 1013.39716 | −0.9373498 | 3.18E−08 | 3.14E−06 |
| KRAS | 0.00177552 | 1 | 1467.31642 | 0.3101724 | 3.29E−08 | 3.22E−06 |
| IRF5 | 0.00025624 | 1 | 293.403812 | 0.38766705 | 3.72E−08 | 3.62E−06 |
| MBOAT2 | 0.00305965 | 1 | 1024.12115 | 0.45174737 | 3.76E−08 | 3.65E−06 |
| B3GNT2 | 0.00022677 | 1 | 876.398933 | −0.4068124 | 3.88E−08 | 3.75E−06 |
| AL118516.1 | 0.00058619 | 1 | 132.98389 | −0.7704049 | 3.99E−08 | 3.84E−06 |
| TMEM9 | 0.00607862 | 1 | 2194.75195 | 0.70249198 | 4.13E−08 | 3.95E−06 |
| ZNFX1 | 0.00265896 | 1 | 2882.71482 | 0.53143764 | 4.77E−08 | 4.55E−06 |
| BOLA1 | 4.46E−05 | 1 | 474.094645 | −0.3507162 | 4.80E−08 | 4.55E−06 |
| BCL2L1 | 0.00028814 | 1 | 4433.11043 | −0.4038039 | 5.35E−08 | 5.05E−06 |
| TPGS2 | 0.00183454 | 1 | 2562.96868 | 0.28208434 | 5.82E−08 | 5.47E−06 |
| DUSP16 | 0.00025142 | 1 | 1288.28823 | 0.45056944 | 6.38E−08 | 5.97E−06 |
| COPA | 0.00033721 | 1 | 11304.2931 | 0.175027 | 6.48E−08 | 6.03E−06 |
| UAP1 | 0.00013111 | 1 | 3165.24782 | −0.3994607 | 7.09E−08 | 6.57E−06 |
| NIF3L1 | 0.00110415 | 1 | 1077.66938 | −0.4457187 | 7.28E−08 | 6.71E−06 |
| TFB1M | 0.00034736 | 1 | 343.339313 | −0.3735648 | 7.60E−08 | 6.97E−06 |
| PRTFDC1 | 0.00041338 | 1 | 1686.24087 | −0.3101259 | 7.76E−08 | 7.09E−06 |
| DNMBP | 0.00297081 | 1 | 2549.55311 | −0.8798521 | 8.12E−08 | 7.39E−06 |
| ROS1 | 0.00634131 | 1 | 43.7351387 | 1.55706675 | 8.22E−08 | 7.42E−06 |
| WNT9A | 0.00618123 | 1 | 489.626349 | 0.74882938 | 8.23E−08 | 7.42E−06 |
| COL18A1 | 0.00738171 | 1 | 968.870779 | 0.99957991 | 8.55E−08 | 7.67E−06 |
| CYB561D1 | 6.86E−05 | 1 | 395.850481 | 0.37735688 | 8.78E−08 | 7.85E−06 |
| LINC00973 | 0.01364192 | 1 | 87.0031739 | −2.4926902 | 1.10E−07 | 9.79E−06 |
| ANTXR2 | 0.00023043 | 1 | 668.955166 | −1.1955164 | 1.12E−07 | 9.88E−06 |
| ASAH2B | 0.00035951 | 1 | 255.460262 | −0.5385721 | 1.12E−07 | 9.91E−06 |
| HCN4 | 0.00032455 | 1 | 17.7779471 | 1.43424842 | 1.23E−07 | 1.08E−05 |
| ZFX | 0.00015612 | 1 | 1440.52418 | 0.31899294 | 1.28E−07 | 1.12E−05 |
| ARHGAP26 | 0.01945511 | 1 | 2338.92918 | −1.6655399 | 1.35E−07 | 1.17E−05 |
| USP33 | 0.00018745 | 1 | 2141.14713 | −0.3216464 | 1.63E−07 | 1.41E−05 |
| SMARCC1 | 7.44E−05 | 1 | 7612.82465 | 0.12431074 | 1.65E−07 | 1.42E−05 |
| MT-ND6 | 0.00085659 | 1 | 17508.9052 | 0.33901237 | 1.77E−07 | 1.52E−05 |
| TGM1 | 0.01874706 | 1 | 103.93445 | 1.96200567 | 1.82E−07 | 1.56E−05 |
| CHST15 | 0.0001438 | 1 | 2279.58854 | 0.28437941 | 1.84E−07 | 1.56E−05 |
| LINC02535 | 0.00395518 | 1 | 65.910811 | −1.8259538 | 1.84E−07 | 1.56E−05 |
| TIMP1 | 0.0054317 | 1 | 1858.91144 | −0.6892317 | 1.89E−07 | 1.59E−05 |
| BTBD2 | 0.00067151 | 1 | 1804.19405 | 0.23077526 | 2.03E−07 | 1.70E−05 |
| KIAA0040 | 0.00072458 | 1 | 642.823515 | −0.7494183 | 2.03E−07 | 1.70E−05 |
| CTNNB1 | 0.0001633 | 1 | 10001.6286 | −0.1865088 | 2.19E−07 | 1.83E−05 |
| KIAA1522 | 0.00118976 | 1 | 8282.32547 | 0.57219857 | 2.22E−07 | 1.85E−05 |
| MTHFD2 | 0.00101203 | 1 | 6311.86892 | −0.2673625 | 2.36E−07 | 1.95E−05 |
| TSPAN17 | 0.0001895 | 1 | 2460.9694 | 0.27922745 | 2.84E−07 | 2.34E−05 |
| MYO1B | 0.0003978 | 1 | 4286.7825 | 0.26692328 | 2.90E−07 | 2.38E−05 |
| LAMB2 | 0.00084533 | 1 | 8018.08951 | 0.92014048 | 3.03E−07 | 2.47E−05 |
| TAF1A | 6.03E−05 | 1 | 131.229224 | −0.5614523 | 3.09E−07 | 2.52E−05 |
| SNRNP70 | 0.00014394 | 1 | 5985.72081 | −0.140776 | 3.18E−07 | 2.58E−05 |
| IRF2BP1 | 0.00076682 | 1 | 1110.1605 | −0.2940541 | 3.32E−07 | 2.68E−05 |
| SF3A3 | 0.00014334 | 1 | 4707.30831 | −0.1666175 | 3.36E−07 | 2.70E−05 |
| IL18R1 | 0.00070705 | 1 | 106.532326 | −0.6000764 | 3.49E−07 | 2.80E−05 |
| CASC19 | 5.37E−06 | 0.19681291 | 145.599565 | −1.9872006 | 3.91E−07 | 3.11E−05 |
| VSIR | 0.00600624 | 1 | 775.010103 | 1.07527989 | 3.92E−07 | 3.11E−05 |
| MED15 | 0.0004144 | 1 | 2708.83392 | 0.20633782 | 3.96E−07 | 3.14E−05 |
| AFF1 | 0.00041005 | 1 | 2864.98335 | 0.41702741 | 4.15E−07 | 3.27E−05 |
| AC022211.2 | 0.00034198 | 1 | 126.000579 | −0.5797446 | 4.38E−07 | 3.42E−05 |
| HSP90AA1 | 0.00032936 | 1 | 113938.384 | −0.3681723 | 4.37E−07 | 3.42E−05 |
| S100A10 | 0.00037888 | 1 | 18163.2041 | 0.396969 | 4.60E−07 | 3.58E−05 |
| SOD1 | 0.00030915 | 1 | 7726.76127 | 0.17482727 | 4.79E−07 | 3.72E−05 |
| HECTD4 | 0.00029392 | 1 | 1974.67548 | 0.25564891 | 4.87E−07 | 3.77E−05 |
| AP3D1 | 0.00232172 | 1 | 7745.74759 | 0.22868577 | 5.21E−07 | 4.02E−05 |
| GPR37L1 | 2.89E−05 | 1 | 6.43839279 | 2.66451095 | 5.50E−07 | 4.22E−05 |
| NUP205 | 0.00014533 | 1 | 6224.65435 | −0.1463035 | 5.75E−07 | 4.40E−05 |
| NF1 | 0.00022227 | 1 | 3755.11899 | −0.2556239 | 5.84E−07 | 4.45E−05 |
| GALE | 0.00082698 | 1 | 2633.71048 | −0.4248476 | 5.861−07 | 4.45E−05 |
| GNAI3 | 4.99E−05 | 1 | 3657.44111 | −0.1146494 | 5.94E−07 | 4.49E−05 |
| SESTD1 | 0.00015743 | 1 | 719.613066 | −0.271006 | 5.99E−07 | 4.51E−05 |
| NUDCD3 | 0.00029078 | 1 | 3982.92692 | 0.21267517 | 6.05E−07 | 4.54E−05 |
| PDHB | 0.00311797 | 1 | 1651.31643 | −0.2901127 | 6.12E−07 | 4.56E−05 |
| TXNRD2 | 0.0001412 | 1 | 887.623989 | 0.23320487 | 6.11E−07 | 4.56E−05 |
| RALBP1 | 0.00026159 | 1 | 3805.2567 | 0.15679101 | 6.47E−07 | 4.80E−05 |
| RAB6A | 0.00078616 | 1 | 3688.03141 | 0.14054931 | 6.89E−07 | 5.10E−05 |
| SLC1A4 | 0.00026401 | 1 | 1839.74729 | 0.5129747 | 7.49E−07 | 5.52E−05 |
| ZBTB5 | 0.00237605 | 1 | 487.088199 | 0.39561125 | 7.68E−07 | 5.64E−05 |
| ZBTB4 | 0.00181865 | 1 | 2523.50968 | 0.40086633 | 7.96E−07 | 5.82E−05 |
| NPAS2 | 0.00037716 | 1 | 1049.06131 | −0.8132666 | 8.14E−07 | 5.93E−05 |
| COX8A | 0.00250693 | 1 | 1706.71719 | −0.3869963 | 8.64E−07 | 6.27E−05 |
| PSAPL1 | 0.00910787 | 1 | 7.85879651 | 3.34768929 | 8.81E−07 | 6.37E−05 |
| MKNK2 | 0.00028537 | 1 | 3003.76202 | 0.29474937 | 9.26E−07 | 6.65E−05 |
| WDR19 | 0.00047119 | 1 | 330.004921 | 0.33914319 | 9.26E−07 | 6.65E−05 |
| DYRK1A | 0.00148657 | 1 | 2403.54865 | 0.25203724 | 9.32E−07 | 6.67E−05 |
| SPATA33 | 0.00025341 | 1 | 395.544744 | 0.37796026 | 9.68E−07 | 6.90E−05 |
| GARNL3 | 0.00687514 | 1 | 134.437366 | 1.01892432 | 1.07E−06 | 7.62E−05 |
| USP39 | 0.00073354 | 1 | 3074.80454 | −0.2442789 | 1.08E−06 | 7.67E−05 |
| DCLRE1C | 0.00051264 | 1 | 921.372209 | −0.2670916 | 1.09E−06 | 7.69E−05 |
| B3GNTL1 | 0.00096141 | 1 | 192.005429 | −0.6142735 | 1.18E−06 | 8.31E−05 |
| AC137932.2 | 0.00651685 | 1 | 22.1512685 | −1.8897674 | 1.19E−06 | 8.36E−05 |
| VDR | 0.01656269 | 1 | 2086.666 | −1.0847801 | 1.21E−06 | 8.46E−05 |
| PRDX6 | 0.0073769 | 1 | 11500.4159 | 0.55675908 | 1.23E−06 | 8.54E−05 |
| FOPNL | 0.00063856 | 1 | 2583.8424 | −0.3333215 | 1.27E−06 | 8.80E−05 |
| NCAPD3 | 0.00031083 | 1 | 3003.65022 | −0.2749187 | 1.29E−06 | 8.91E−05 |
| SLC26A4-AS1 | 0.00185793 | 1 | 19.8761444 | −1.624412 | 1.33E−06 | 9.17E−05 |
| RAPGEF1 | 0.00041136 | 1 | 3838.08663 | 0.34944928 | 1.45E−06 | 9.93E−05 |
| SLC19A3 | 0.01854191 | 1 | 41.3793859 | 2.19172229 | 1.45E−06 | 9.93E−05 |
| SLC16A4 | 0.0002606 | 1 | 269.71922 | 0.55579105 | 1.51E−06 | 0.00010309 |
| DIDO1 | 0.00175329 | 1 | 4037.52657 | 0.24977071 | 1.52E−06 | 0.00010365 |
| NUDT22 | 0.00015058 | 1 | 478.251535 | −0.3036275 | 1.54E−06 | 0.00010425 |
| SLC41A2 | 0.00053051 | 1 | 745.40337 | 0.89948355 | 1.56E−06 | 0.00010522 |
| EVC | 0.00057826 | 1 | 503.914536 | 0.65366141 | 1.63E−06 | 0.00010958 |
| FOXO3 | 0.00104514 | 1 | 1248.92799 | 0.22977876 | 1.66E−06 | 0.00011109 |
| ZSWIM6 | 0.00017601 | 1 | 1052.37471 | 0.24325928 | 1.70E−06 | 0.00011364 |
| TMEM208 | 0.00566361 | 1 | 1375.12801 | 0.37624828 | 1.95E−06 | 0.00013021 |
| FLT4 | 0.0021871 | 1 | 216.255577 | −0.6023299 | 1.97E−06 | 0.00013058 |
| HLA-DMA | 0.00415659 | 1 | 144.148592 | 0.85958936 | 1.98E−06 | 0.00013058 |
| TLR3 | 0.00384398 | 1 | 145.22722 | 0.92874365 | 1.97E−06 | 0.00013058 |
| ZBTB47 | 0.00161406 | 1 | 355.845304 | 0.54032774 | 2.05E−06 | 0.0001347 |
| MCM2 | 0.00020934 | 1 | 4183.63305 | 0.16313839 | 2.14E−06 | 0.00014056 |
| AC112220.2 | 0.0002713 | 1 | 162.823033 | 0.5768398 | 2.20E−06 | 0.00014351 |
| NEK3 | 0.00461617 | 1 | 175.017234 | −0.6828855 | 2.23E−06 | 0.00014497 |
| PGLS | 0.00039266 | 1 | 894.615199 | 0.45731083 | 2.28E−06 | 0.00014797 |
| GON7 | 0.00023737 | 1 | 342.861177 | −0.4247737 | 2.33E−06 | 0.00015104 |
| FBXW4 | 0.00038102 | 1 | 1160.79304 | 0.33723616 | 2.44E−06 | 0.0001576 |
| ARHGEF37 | 0.00065721 | 1 | 304.320834 | 0.86432662 | 2.52E−06 | 0.00016192 |
| ADAMTS15 | 0.000154 | 1 | 158.237612 | 0.5493691 | 2.61E−06 | 0.00016727 |
| RASA1 | 0.0010874 | 1 | 1350.07114 | −0.2583216 | 2.66E−06 | 0.00016968 |
| HPS | 0.00679321 | 1 | 1219.34723 | 0.4099462 | 2.81E−06 | 0.00017905 |
| SAMD1 | 0.0031772 | 1 | 1959.00559 | 0.23062872 | 2.98E−06 | 0.00018889 |
| TSC22D2 | 0.00015837 | 1 | 979.731992 | 0.22479712 | 2.98E−06 | 0.00018889 |
| PSG9 | 0.01162309 | 1 | 8.47896773 | 2.55520046 | 3.08E−06 | 0.00019417 |
| EFCAB14 | 0.00013618 | 1 | 3792.45501 | 0.12090898 | 3.12E−06 | 0.00019613 |
| GNPTG | 0.00527664 | 1 | 700.194977 | 0.66267675 | 3.19E−06 | 0.00019973 |
| SNX9 | 0.00024919 | 1 | 2068.13781 | −0.181735 | 3.24E−06 | 0.00020224 |
| PGPEP1 | 0.0008197 | 1 | 543.209941 | −0.4548295 | 3.29E−06 | 0.00020477 |
| TTC3 | 0.00047812 | 1 | 8686.98219 | 0.38297437 | 3.35E−06 | 0.00020816 |
| POLR2H | 0.00074113 | 1 | 1895.07334 | −0.3441469 | 3.37E−06 | 0.00020902 |
| SPTBN5 | 0.00505564 | 1 | 130.664704 | 0.73807723 | 3.66E−06 | 0.00022602 |
| CHMP4B | 0.00028469 | 1 | 3721.73238 | 0.16935856 | 3.74E−06 | 0.00023004 |
| AP001053.1 | 0.02049229 | 1 | 24.9018548 | 1.97016307 | 3.78E−06 | 0.00023208 |
| TRIM26 | 0.00052284 | 1 | 2066.68379 | 0.16246479 | 3.83E−06 | 0.00023412 |
| MBOAT7 | 0.00499733 | 1 | 4054.09464 | 0.31388047 | 3.84E−06 | 0.00023439 |
| CAT | 0.00259094 | 1 | 1328.05392 | 0.27470448 | 4.06E−06 | 0.00024678 |
| CCDC144NL-AS1 | 0.00079566 | 1 | 6.63294089 | 2.70850786 | 4.12E−06 | 0.00024997 |
| SGMS2 | 0.00030489 | 1 | 959.310773 | 0.20887177 | 4.22E−06 | 0.00025521 |
| EBAG9 | 0.00025798 | 1 | 777.501454 | −0.2439261 | 4.34E−06 | 0.00026167 |
| FAM86DP | 0.00584917 | 1 | 271.821472 | −0.772142 | 4.50E−06 | 0.00027055 |
| AC007773.1 | 0.00047442 | 1 | 29.636776 | −1.0102412 | 4.54E−06 | 0.0002711 |
| TCF25 | 0.00053261 | 1 | 2820.29143 | 0.41073652 | 4.54E−06 | 0.0002711 |
| RICTOR | 0.00053529 | 1 | 1684.68452 | −0.4446802 | 4.69E−06 | 0.00027905 |
| INPPL1 | 0.00182541 | 1 | 5161.30977 | 0.2614561 | 4.81E−06 | 0.00028571 |
| AL035252.3 | 0.00021628 | 1 | 39.6060138 | −1.1382666 | 4.91E−06 | 0.00029021 |
| ELOF1 | 0.00232927 | 1 | 1042.98651 | −0.2355907 | 4.92E−06 | 0.00029021 |
| PSMB1 | 0.00031161 | 1 | 4869.95166 | −0.1266556 | 4.93E−06 | 0.00029021 |
| MEGF6 | 0.00820569 | 1 | 251.404473 | 0.92187863 | 5.12E−06 | 0.00030039 |
| SNRPD2 | 0.00070918 | 1 | 5643.42853 | −0.2140815 | 5.18E−06 | 0.00030294 |
| HSP90AA2P | 0.000141 | 1 | 63.6866178 | −0.6377556 | 5.41E−06 | 0.00031544 |
| SORCS2 | 0.00087054 | 1 | 6.06145048 | 2.20361265 | 5.55E−06 | 0.00032296 |
| ETV1 | 0.03291968 | 1 | 955.590317 | 2.8280534 | 5.68E−06 | 0.00032862 |
| PUM1 | 0.00090914 | 1 | 2973.81678 | 0.18005386 | 5.68E−06 | 0.00032862 |
| ECT2 | 0.00407161 | 1 | 6560.4643 | −0.6009709 | 5.74E−06 | 0.00033094 |
| TCF3 | 0.00056619 | 1 | 3347.72557 | 0.25734666 | 5.92E−06 | 0.00034047 |
| ABHD4 | 0.00535965 | 1 | 6747.99353 | 0.74448477 | 6.06E−06 | 0.00034693 |
| PHLDA1 | 0.03637532 | 1 | 1830.86137 | −1.7961742 | 6.07E−06 | 0.00034693 |
| AKR1A1 | 0.0022787 | 1 | 1924.03491 | 0.27129999 | 6.16E−06 | 0.00035119 |
| SLC38A7 | 0.00055263 | 1 | 1179.58877 | 0.3768454 | 6.27E−06 | 0.00035645 |
| TMEM59 | 0.00602299 | 1 | 3565.88358 | 0.38760127 | 6.38E−06 | 0.00036159 |
| BRCC3 | 0.00260667 | 1 | 1622.79118 | 0.22818269 | 6.40E−06 | 0.00036186 |
| WFDC21P | 7.35E−05 | 1 | 56.073958 | 1.51242465 | 6.58E−06 | 0.00037114 |
| IL1RL1 | 0.00071046 | 1 | 4.38674732 | −3.9565012 | 6.74E−06 | 0.00037826 |
| PIP5K1C | 0.00067157 | 1 | 1161.71518 | 0.28216783 | 6.74E−06 | 0.00037826 |
| TATDN3 | 0.00019811 | 1 | 479.07222 | −0.2705821 | 6.77E−06 | 0.00037879 |
| BCKDHB | 0.00161516 | 1 | 542.677882 | 0.29271805 | 7.35E−06 | 0.00040834 |
| HSPA1A | 0.00652112 | 1 | 572.251164 | 0.42644192 | 7.36E−06 | 0.00040834 |
| WDR41 | 0.00050249 | 1 | 1769.04375 | 0.23671661 | 7.35E−06 | 0.00040834 |
| PHPT1 | 0.00240582 | 1 | 1520.75559 | 0.38962036 | 7.53E−06 | 0.00041634 |
| FANCM | 0.00055619 | 1 | 583.779799 | −0.2921127 | 7.85E−06 | 0.00043331 |
| AL645608.7 | 6.01E−05 | 1 | 30.3267712 | 0.88063918 | 7.89E−06 | 0.00043406 |
| SHTN1 | 0.00059336 | 1 | 4811.97769 | 0.29822178 | 7.92E−06 | 0.00043456 |
| TMEM135 | 0.00097279 | 1 | 1124.72344 | −0.4540785 | 8.19E−06 | 0.00044801 |
| CCT8 | 0.0008745 | 1 | 9190.86438 | −0.2789827 | 8.39E−06 | 0.00045797 |
| FAM13B | 0.00912076 | 1 | 1136.75251 | 0.68518711 | 8.46E−06 | 0.00045895 |
| SORBS3 | 0.00024988 | 1 | 2188.42922 | 0.16545425 | 8.45E−06 | 0.00045895 |
| RCN3 | 0.00018869 | 1 | 13.3723774 | −1.3021645 | 8.52E−06 | 0.00046126 |
| OLMALINC | 0.00264777 | 1 | 569.319798 | −1.112309 | 8.67E−06 | 0.00046794 |
| BCL2L12 | 0.00064724 | 1 | 921.553303 | −0.27305 | 8.85E−06 | 0.00047643 |
| THAP2 | 0.00011696 | 1 | 86.3013891 | −0.5238522 | 9.09E−06 | 0.00048797 |
| PPDPF | 0.00704778 | 1 | 6437.74521 | 0.35900249 | 1.00E−05 | 0.00053699 |
| SHISAL1 | 0.00029511 | 1 | 30.5623552 | −0.9539371 | 1.03E−05 | 0.0005513 |
| RNF214 | 0.0003198 | 1 | 474.03635 | −0.2719173 | 1.05E−05 | 0.00056164 |
| ST6GALNAC2 | 0.00022264 | 1 | 27.7147825 | 1.15167426 | 1.08E−05 | 0.00057325 |
| DGAT2 | 0.00765968 | 1 | 324.632695 | 0.59409456 | 1.20E−05 | 0.00063408 |
| AADAC | 0.0347131 | 1 | 116.485733 | −1.7947492 | 1.29E−05 | 0.00067825 |
| MUC1 | 0.01803224 | 1 | 3041.47819 | 1.22628249 | 1.29E−05 | 0.00067825 |
| TLNRD1 | 0.00084943 | 1 | 1657.43479 | −0.2801921 | 1.32E−05 | 0.00059153 |
| AGRN | 0.00091039 | 1 | 20444.5751 | 0.43847362 | 1.35E−05 | 0.00070348 |
| MT-ND4 | 0.00200381 | 1 | 133071.704 | 0.25528277 | 1.35E−05 | 0.00070348 |
| STT3B | 0.00096253 | 1 | 6505.89903 | 0.20667864 | 1.35E−05 | 0.00070348 |
| MED29 | 0.00054361 | 1 | 1875.95888 | 0.22053957 | 1.39E−05 | 0.00072145 |
| C3orf52 | 0.00031959 | 1 | 308.698405 | 0.31152407 | 1.43E−05 | 0.00074252 |
| AL592071.1 | 0.00034369 | 1 | 12.2691421 | −1.3576273 | 1.48E−05 | 0.00076753 |
| INPP5F | 0.00397304 | 1 | 792.417734 | −0.4692675 | 1.51E−05 | 0.00078199 |
| CYB5R1 | 0.00087139 | 1 | 1082.33777 | 0.27470045 | 1.53E−05 | 0.00078642 |
| SNRPA | 0.00068977 | 1 | 2235.64857 | −0.1966307 | 1.57E−05 | 0.00080707 |
| NKRF | 0.00011917 | 1 | 539.692195 | −0.2252161 | 1.60E−05 | 0.00081956 |
| LINC01589 | 0.00026323 | 1 | 45.612839 | −0.7838284 | 1.61E−05 | 0.00082112 |
| KLF11 | 0.00109506 | 1 | 1344.64732 | 0.43522307 | 1.61E−05 | 0.0008219 |
| PCSK7 | 0.00079798 | 1 | 735.763603 | 0.59726717 | 1.62E−05 | 0.00082241 |
| DTX3L | 0.00178298 | 1 | 1556.04056 | 0.46133336 | 1.63E−05 | 0.00082503 |
| TUBA4A | 0.00157199 | 1 | 14050.122 | 0.55072014 | 1.63E−05 | 0.00082503 |
| DUSP3 | 0.00113024 | 1 | 3926.10988 | 0.43522664 | 1.64E−05 | 0.0008298 |
| GSTO1 | 0.00234765 | 1 | 2923.59508 | −0.3538999 | 1.66E−05 | 0.00083362 |
| MB | 0.00018247 | 1 | 95.2682361 | 1.52701802 | 1.67E−05 | 0.0008384 |
| LIMS2 | 0.00540758 | 1 | 12.8689028 | 1.5178917 | 1.81E−05 | 0.00090667 |
| STAM | 0.00166252 | 1 | 1806.07503 | −0.2423173 | 1.88E−05 | 0.00093911 |
| CEP41 | 0.00072464 | 1 | 381.115112 | −0.3471224 | 1.91E−05 | 0.00095383 |
| C1orf52 | 0.00064111 | 1 | 418.326955 | −0.3496669 | 1.98E−05 | 0.00098446 |
| COL4A4 | 0.0130608 | 1 | 3964.77633 | 0.96834104 | 2.00E−05 | 0.00099316 |
| PRRC2B | 0.00079777 | 1 | 5402.42401 | 0.23269296 | 2.05E−05 | 0.00101236 |
| LZTR1 | 0.00574054 | 1 | 313.607071 | 0.51598916 | 2.18E−05 | 0.0010756 |
| HABP4 | 0.00964423 | 1 | 374.14227 | 0.62217021 | 2.22E−05 | 0.00109373 |
| VGF | 0.01443716 | 1 | 85.9695342 | −2.1111202 | 2.23E−05 | 0.00109422 |
| CD151 | 0.00100884 | 1 | 5758.20811 | 0.48702645 | 2.28E−05 | 0.00111596 |
| ARHGAP19 | 0.00102784 | 1 | 1028.95945 | −0.4490579 | 2.33E−05 | 0.00113934 |
| AC092868.1 | 0.00242683 | 1 | 32.5364795 | −1.1367778 | 2.43E−05 | 0.0011845 |
| AC016065.1 | 0.00042541 | 1 | 128.62419 | −0.4710451 | 2.49E−05 | 0.0012134 |
| BEST3 | 0.0005843 | 1 | 12.260935 | −1.4127516 | 2.54E−05 | 0.00123123 |
| PRUNE2 | 0.00251849 | 1 | 17.2864898 | 1.33956646 | 2.55E−05 | 0.00123577 |
| TGM2 | 0.00027271 | 1 | 23564.4245 | −1.4815952 | 2.56E−05 | 0.00123577 |
| MEIS3 | 0.00089573 | 1 | 124.804434 | 0.52631718 | 2.58E−05 | 0.00124405 |
| BAG3 | 0.00249021 | 1 | 3473.13886 | −0.2451183 | 2.67E−05 | 0.00128222 |
| AC100861.1 | 0.0004382 | 1 | 144.825297 | −0.4816449 | 2.68E−05 | 0.00128413 |
| INCENP | 0.00081742 | 1 | 2402.51041 | −0.1621886 | 2.69E−05 | 0.00128534 |
| SEPTIN8 | 0.00091355 | 1 | 2666.80501 | 0.26319567 | 2.71E−05 | 0.00129417 |
| AC008443.1 | 0.00128964 | 1 | 77.9311489 | −0.6708673 | 2.72E−05 | 0.00129517 |
| APOO | 0.00119175 | 1 | 652.000631 | −0.3416284 | 2.79E−05 | 0.00132436 |
| CCDC68 | 0.01445917 | 1 | 629.197288 | −0.9757423 | 2.85E−05 | 0.00134913 |
| POLR2C | 0.00084864 | 1 | 3292.03414 | −0.2537331 | 2.87E−05 | 0.00135708 |
| ORC2 | 0.00269245 | 1 | 1317.14232 | −0.3352992 | 2.89E−05 | 0.00136098 |
| ARHGAP12 | 0.01148124 | 1 | 2542.73406 | −0.6448194 | 2.89E−05 | 0.00136118 |
| PIEZO1 | 0.00300718 | 1 | 7897.81045 | 0.51608207 | 2.92E−05 | 0.00137144 |
| ANAPC10 | 0.00255846 | 1 | 91.6401057 | −0.5640369 | 2.94E−05 | 0.00137938 |
| FSTL3 | 0.02359869 | 1 | 3818.33057 | 0.90530728 | 2.96E−05 | 0.00138469 |
| COX5B | 0.00441689 | 1 | 2580.06393 | 0.18138585 | 2.98E−05 | 0.00139045 |
| RARB | 0.00028081 | 1 | 215.73661 | 0.36627363 | 3.05E−05 | 0.00141683 |
| NFS1 | 0.00061193 | 1 | 947.954921 | −0.231595 | 3.09E−05 | 0.00143219 |
| COIL | 0.00340813 | 1 | 1148.71538 | −0.2499911 | 3.14E−05 | 0.00145454 |
| CASP1 | 0.01777294 | 1 | 23.3354288 | 1.60075102 | 3.20E−05 | 0.00147736 |
| MPZL1 | 0.00055981 | 1 | 8625.28288 | 0.10332047 | 3.23E−05 | 0.00148924 |
| COA6-AS1 | 0.0018419 | 1 | 31.8700695 | −0.8577244 | 3.35E−05 | 0.00154203 |
| BTBD8 | 0.00071225 | 1 | 102.842417 | −0.6662375 | 3.37E−05 | 0.00154657 |
| ABCA1 | 0.00089669 | 1 | 728.648012 | −0.3676142 | 3.43E−05 | 0.00156912 |
| TULP4 | 0.00109878 | 1 | 1007.0657 | 0.25075471 | 3.46E−05 | 0.0015794 |
| LLGL1 | 0.00175923 | 1 | 1748.58512 | 0.17772418 | 3.49E−05 | 0.00158652 |
| SLC8A1 | 0.01736146 | 1 | 65.3601825 | −1.0390643 | 3.49E−05 | 0.00158652 |
| ZNF385A | 0.00177588 | 1 | 677.355149 | 0.36141625 | 3.52E−05 | 0.00159817 |
| RUFY1 | 0.00035852 | 1 | 1240.39461 | 0.16739977 | 3.60E−05 | 0.0016278 |
| SLC39A13 | 0.00545505 | 1 | 532.856614 | 0.35307421 | 3.64E−05 | 0.0016457 |
| ST3GAL5 | 0.04291945 | 1 | 948.069295 | −1.5293136 | 3.70E−05 | 0.00166651 |
| BCL2L11 | 0.00299631 | 1 | 606.700798 | 0.35568906 | 3.91E−05 | 0.00175233 |
| RLF | 0.00117008 | 1 | 951.085705 | −0.3148679 | 3.90E−05 | 0.00175233 |
| PAM | 0.00839729 | 1 | 4385.81391 | 0.6648739 | 3.92E−05 | 0.0017548 |
| RNF19A | 0.00119332 | 1 | 980.547004 | 0.49608194 | 4.15E−05 | 0.00185164 |
| PPARA | 0.0021496 | 1 | 502.908218 | −0.434805 | 4.17E−05 | 0.00185586 |
| SLC25A11 | 0.00082114 | 1 | 1918.92325 | 0.1713884 | 4.17E−05 | 0.00185586 |
| GK5 | 0.00207732 | 1 | 958.761614 | −0.2489216 | 4.21E−05 | 0.00186889 |
| ATF1 | 0.00257641 | 1 | 744.147387 | −0.3964032 | 4.26E−05 | 0.0018845 |
| CLDN3 | 0.00559634 | 1 | 147.227746 | 0.57523731 | 4.29E−05 | 0.00189246 |
| HSP90AB1 | 0.00324167 | 1 | 76597.6265 | −0.1610468 | 4.29E−05 | 0.00189246 |
| AC125807.2 | 0.00089341 | 1 | 554.979377 | 0.37030999 | 4.30E−05 | 0.00189326 |
| GRID1 | 0.0128413 | 1 | 93.2278986 | 0.7863754 | 4.44E−05 | 0.00194666 |
| MAGI2 | 0.00063212 | 1 | 137.396102 | −0.4332134 | 4.48E−05 | 0.00196364 |
| SHKBP1 | 0.00548438 | 1 | 1768.0225 | 0.29102464 | 4.56E−05 | 0.00199421 |
| DGCR6L | 0.00615212 | 1 | 762.439429 | 0.28566631 | 4.72E−05 | 0.00206008 |
| TBX2 | 0.00127459 | 1 | 84.4456215 | −0.6037297 | 5.00E−05 | 0.00217716 |
| NCOA5 | 0.00097868 | 1 | 1983.52185 | 0.18047677 | 5.02E−05 | 0.00217776 |
| SLC35G1 | 0.00087399 | 1 | 564.638605 | −0.2863717 | 5.12E−05 | 0.0022168 |
| ZNF200 | 0.00077501 | 1 | 457.746719 | −0.3201727 | 5.16E−05 | 0.0022288 |
| LINC01119 | 0.00493626 | 1 | 11.0574432 | −1.4991783 | 5.30E−05 | 0.00228493 |
| AC017100.1 | 0.00050356 | 1 | 52.6540457 | −0.6836933 | 5.77E−05 | 0.00248268 |
| GRIP1 | 0.00093822 | 1 | 320.677051 | 0.38251237 | 5.83E−05 | 0.00250447 |
| SNRNP48 | 0.00524536 | 1 | 967.419802 | −0.3561033 | 5.87E−05 | 0.00251418 |
| ARAP1 | 0.00138943 | 1 | 1778.84404 | −0.2279113 | 6.05E−05 | 0.00258742 |
| MT-ND4L | 0.00380149 | 1 | 15363.2011 | 0.22386559 | 6.10E−05 | 0.00260569 |
| LSR | 0.00292974 | 1 | 4949.10574 | 0.17092812 | 6.14E−05 | 0.0026159 |
| EMP2 | 0.01600825 | 1 | 1327.90401 | 0.52361874 | 6.19E−05 | 0.00263039 |
| AFAP1 | 0.0198259 | 1 | 1813.3577 | 0.62926273 | 6.24E−05 | 0.00264803 |
| CSMD3 | 0.03547165 | 1 | 49.9190325 | −1.7084017 | 6.47E−05 | 0.00273915 |
| GRN | 0.00273934 | 1 | 5725.65827 | 0.69464347 | 6.64E−05 | 0.00280322 |
| RPL29 | 0.00060413 | 1 | 14100.5917 | −0.0815617 | 6.78E−05 | 0.00285819 |
| GMNN | 0.00218667 | 1 | 1797.33025 | −0.2253827 | 6.82E−05 | 0.00285986 |
| PFKL | 0.00133417 | 1 | 4870.1178 | 0.19311695 | 6.82E−05 | 0.00285986 |
| ST5 | 0.00023813 | 1 | 265.487647 | 0.29570916 | 6.83E−05 | 0.00285986 |
| TNFRSF21 | 0.00548 | 1 | 2911.88313 | −0.2182826 | 6.86E−05 | 0.00286626 |
| AC108752.1 | 0.01624774 | 1 | 49.8714582 | 2.94115168 | 6.97E−05 | 0.00290596 |
| GCNA | 0.031708 | 1 | 29.7096514 | −1.64259 | 7.08E−05 | 0.00294672 |
| CLDN2 | 0.03650137 | 1 | 28.5235351 | 1.998922 | 7.35E−05 | 0.00305141 |
| L1CAM | 0.04286629 | 1 | 1256.70539 | 2.04410854 | 7.57E−05 | 0.00313916 |
| PPP1R14C | 0.00117089 | 1 | 305.839627 | 0.32758285 | 7.64E−05 | 0.0031628 |
| BHLHE40 | 0.02135716 | 1 | 3035.65129 | −0.8069877 | 7.74E−05 | 0.00319702 |
| QRICH2 | 0.01317369 | 1 | 57.2447993 | 0.74256483 | 7.99E−05 | 0.00329119 |
| GBA2 | 0.00139632 | 1 | 853.315952 | −0.2842531 | 8.02E−05 | 0.00329852 |
| KCNN4 | 0.00268534 | 1 | 766.862061 | −0.4750561 | 8.15E−05 | 0.00334532 |
| SIRT7 | 0.00135158 | 1 | 1073.90753 | 0.27680341 | 8.38E−05 | 0.00343282 |
| CAPZA1 | 0.00175267 | 1 | 7534.19945 | −0.2691093 | 8.53E−05 | 0.00348549 |
| ZNRF3 | 0.0101783 | 1 | 850.209592 | 0.43720532 | 8.55E−05 | 0.00348549 |
| GMEB1 | 0.00039059 | 1 | 667.630432 | −0.1783758 | 8.87E−05 | 0.00360868 |
| LIPA | 0.0100183 | 1 | 3260.96957 | 0.30894096 | 8.89E−05 | 0.00361243 |
| KLC1 | 0.00569088 | 1 | 963.755251 | 0.30099764 | 9.33E−05 | 0.00378129 |
| CDC27 | 0.00189676 | 1 | 4587.67744 | −0.1576233 | 9.54E−05 | 0.00386151 |
| Individual RNAseq data for H2122 (top) and H2030 (bottom) cells that were used to | | | | | | |
| generate the combined differentially expressed genes (DEG) for H2122 and H2030. | | | | | | |
| TNS4 | 0.00011619 | 1 | 13934.1333 | −1.259717 | 3.25E−283 | 7.27E−280 |
| KRT17 | 0.00075533 | 1 | 21539.6525 | 1.21883568 | 5.51E−247 | 1.08E−243 |
| APOL1 | 0.00302981 | 1 | 3321.24283 | 1.85521929 | 2.01E−244 | 3.49E−241 |
| DCLK1 | 0.00018159 | 1 | 978.163442 | −2.5845273 | 1.80E−242 | 2.81E−239 |
| MCFD2 | 2.05E−07 | 0.00666448 | 6488.03922 | −1.1318172 | 3.52E−214 | 5.00E−211 |
| MYEOV | 0.00027619 | 1 | 4207.05736 | −1.4879504 | 6.19E−212 | 8.07E−209 |
| SYNPO | 0.00028195 | 1 | 2644.80376 | 1.44298177 | 2.31E−204 | 2.77E−201 |
| MYH14 | 0.00279485 | 1 | 8292.37911 | 1.35915859 | 3.62E−204 | 4.05E−201 |
| MAP2 | 1.29E−06 | 0.0418434 | 1974.10933 | 1.66664783 | 7.89E−197 | 8.23E−194 |
| ITGA2 | 0.00236194 | 1 | 3580.25219 | −1.5420463 | 1.18E−195 | 1.16E−192 |
| PHLDA1 | 0.00503237 | 1 | 2503.48101 | −2.1152276 | 8.17E−193 | 7.52E−190 |
| DMBT1 | 1.52E−05 | 0.49423339 | 2431.95812 | −1.6758652 | 8.93E−191 | 7.76E−188 |
| TRIM29 | 0.00022775 | 1 | 7552.69288 | 1.10126291 | 2.11E−189 | 1.74E−186 |
| ETV4 | 9.77E−06 | 0.31756765 | 467.437914 | −4.4673675 | 9.87E−187 | 7.72E−184 |
| CALB2 | 6.39E−05 | 1 | 806.127758 | −2.3568042 | 1.26E−171 | 9.36E−169 |
| UBASH3B | 8.12E−07 | 0.02641223 | 828.697703 | −2.2926413 | 1.46E−170 | 1.04E−167 |
| ABHD2 | 0.00011801 | 1 | 13083.0289 | 0.91087349 | 2.16E−170 | 1.47E−167 |
| ARNT2 | 0.0020237 | 1 | 1017.52272 | −2.0797676 | 2.35E−163 | 1.53E−160 |
| CTSL | 9.33E−07 | 0.03035126 | 13002.9177 | −0.8891202 | 1.56E−162 | 9.78E−160 |
| AREG | 0.0004228 | 1 | 2611.98229 | −1.3253316 | 2.37E−159 | 1.43E−156 |
| RGS2 | 1.32E−05 | 0.42775489 | 1328.80181 | −1.7989607 | 2.98E−154 | 1.73E−151 |
| EPHA2 | 2.41E−05 | 0.78125856 | 4188.18198 | −1.14056 | 1.27E−144 | 7.11E−142 |
| LCN2 | 3.24E−05 | 1 | 2045.57233 | 1.35483777 | 1.20E−139 | 6.46E−137 |
| B2M | 0.00041432 | 1 | 18976.4995 | 0.76599833 | 4.45E−133 | 2.32E−130 |
| NCF2 | 0.00059842 | 1 | 1448.05415 | 1.45978667 | 1.95E−131 | 9.86E−129 |
| ETV5 | 1.16E−07 | 0.00378325 | 324.607707 | −4.0360322 | 2.59E−131 | 1.26E−128 |
| NEDD9 | 3.26E−05 | 1 | 1299.82449 | 1.62342264 | 4.92E−129 | 2.33E−126 |
| FOSL1 | 3.26E−05 | 1 | 1682.51191 | −1.5256709 | 2.00E−128 | 9.19E−126 |
| LTBP4 | 0.00024846 | 1 | 1656.42344 | 1.55570014 | 2.57E−128 | 1.15E−125 |
| PSAP | 0.00034408 | 1 | 24641.3901 | 0.78019764 | 9.74E−126 | 4.23E−123 |
| KRT18 | 2.66E−05 | 0.86264043 | 23513.6693 | −0.8756781 | 1.44E−125 | 6.10E−123 |
| SPRY4 | 0.0004149 | 1 | 306.599859 | −3.9555144 | 1.55E−123 | 6.39E−121 |
| BACE2 | 0.00141722 | 1 | 20954.9472 | 0.79158426 | 2.86E−123 | 1.15E−120 |
| BMF | 1.59E−05 | 0.51699073 | 460.516896 | 2.73688814 | 5.54E−122 | 2.16E−119 |
| EREG | 0.00748994 | 1 | 1985.17369 | −1.6278786 | 1.75E−120 | 6.69E−118 |
| SAT1 | 5.31E−06 | 0.17253136 | 1823.36044 | −1.320312 | 3.27E−119 | 1.22E−116 |
| S100A9 | 8.39E−05 | 1 | 2338.6404 | −1.1870863 | 1.24E−117 | 4.51E−115 |
| LAMB2 | 4.43E−05 | 1 | 6031.00944 | 0.98804676 | 1.70E−113 | 6.06E−111 |
| MATN2 | 1.11E−05 | 0.36191439 | 1005.34024 | 1.5160444 | 2.32E−109 | 8.06E−107 |
| IGFBP3 | 0.00026095 | 1 | 1167.60532 | 1.47013038 | 1.83E−108 | 6.23E−106 |
| UGT1A6 | 0.00045262 | 1 | 1672.88276 | 1.25267957 | 1.01E−107 | 3.38E−105 |
| SRI | 0.00076313 | 1 | 3479.9967 | 0.99898875 | 3.70E−106 | 1.21E−103 |
| EPGN | 0.0046153 | 1 | 2048.33966 | −1.3301793 | 2.29E−104 | 7.30E−102 |
| PLXNA2 | 2.49E−06 | 0.08090116 | 1357.99579 | 1.33079573 | 6.88E−104 | 2.15E−101 |
| EPB41L1 | 4.96E−06 | 0.16139678 | 2386.24193 | 1.04984904 | 7.39E−104 | 2.27E−101 |
| KRT80 | 4.70E−06 | 0.15271255 | 12644.6682 | 0.69853943 | 6.86E−99 | 2.06E−96 |
| ACSL5 | 0.00023227 | 1 | 339.961162 | −2.6307921 | 2.98E−97 | 8.81E−95 |
| SMOX | 0.00381346 | 1 | 5524.64445 | −0.9583757 | 3.05E−97 | 8.84E−95 |
| ITGA6 | 1.84E−05 | 0.59936329 | 4253.32609 | −0.8397422 | 1.00E−95 | 2.85E−93 |
| SLAMF9 | 2.28E−05 | 0.74202284 | 695.677803 | −1.8345989 | 1.23E−95 | 3.43E−93 |
| ETV | 0.00058316 | 1 | 298.398626 | −2.8754215 | 2.40E−95 | 6.59E−93 |
| AKR1C1 | 0.00183844 | 1 | 29916.8797 | 0.89932695 | 8.34E−95 | 2.25E−92 |
| AQP3 | 6.95E−05 | 1 | 5364.94621 | 1.00327059 | 3.75E−94 | 9.95E−92 |
| MCAM | 0.00168859 | 1 | 613.279555 | 1.91722203 | 1.30E−93 | 3.39E−91 |
| LCAL1 | 0.00114865 | 1 | 3297.80301 | 0.97154581 | 3.05E−92 | 7.82E−90 |
| BCAS1 | 0.00011256 | 1 | 1685.48428 | 1.21825421 | 4.61E−92 | 1.16E−89 |
| LAPTM4A | 4.38E−05 | 1 | 6067.89513 | 0.79638694 | 1.73E−89 | 4.29E−87 |
| ALDH3B1 | 0.0030803 | 1 | 6365.82618 | 0.85202061 | 2.46E−89 | 6.02E−87 |
| AKR1C2 | 3.31E−05 | 1 | 68649.2689 | 0.67399959 | 5.29E−88 | 1.27E−85 |
| PTGES | 0.00275021 | 1 | 12769.9939 | −0.7271556 | 6.34E−88 | 1.50E−85 |
| POLM | 7.17E−05 | 1 | 1390.86749 | −1.2417091 | 4.86E−87 | 1.14E−84 |
| LGALS3BP | 2.39E−05 | 0.77723954 | 7774.1204 | 0.77479522 | 1.20E−86 | 2.77E−84 |
| DAPK1 | 0.00133148 | 1 | 1725.4475 | 1.09830081 | 3.37E−85 | 7.65E−83 |
| KRT4 | 0.01061186 | 1 | 233.468791 | 4.18261207 | 3.94E−85 | 8.82E−83 |
| LDLR | 0.00012646 | 1 | 2963.53 | −0.8853924 | 3.40E−84 | 7.49E−82 |
| CD36 | 0.00320186 | 1 | 25248.2952 | 0.69937053 | 7.64E−84 | 1.66E−81 |
| BMP2 | 8.46E−05 | 1 | 3679.51119 | 0.91194694 | 1.55E−83 | 3.32E−81 |
| THBS1 | 0.00037118 | 1 | 3950.16951 | 1.07429078 | 3.24E−82 | 6.84E−80 |
| DUSP6 | 0.00284926 | 1 | 205.458092 | −4.6179666 | 8.90E−82 | 1.86E−79 |
| ANXA8 | 3.83E−05 | 1 | 554.326627 | 1.7919574 | 1.87E−81 | 3.85E−79 |
| ABLIM1 | 0.00012859 | 1 | 15102.7118 | 0.5791493 | 5.57E−81 | 1.13E−78 |
| DOCK4 | 1.05E−06 | 0.03412768 | 587.092381 | −1.6631419 | 1.39E−80 | 2.78E−78 |
| TFF1 | 0.00070164 | 1 | 3728.51322 | −1.1471351 | 1.98E−80 | 3.92E−78 |
| NCEH1 | 1.38E−06 | 0.04505315 | 2005.78841 | −0.9640248 | 4.84E−80 | 9.47E−78 |
| PAQR5 | 0.00294423 | 1 | 1857.19013 | −1.0871453 | 2.82E−79 | 5.44E−77 |
| FURIN | 0.00022029 | 1 | 10132.1337 | −0.7181085 | 3.25E−79 | 6.19E−77 |
| ARRDC4 | 0.00013376 | 1 | 2139.04945 | 1.18508758 | 6.74E−79 | 1.27E−76 |
| SPINK1 | 0.00317665 | 1 | 203.05872 | 3.97809836 | 8.17E−79 | 1.52E−76 |
| CEMIP2 | 1.43E−05 | 0.46356897 | 3161.14976 | −0.834082 | 2.08E−78 | 3.83E−76 |
| GRN | 0.00011556 | 1 | 4330.55628 | 0.77663866 | 2.79E−78 | 5.07E−76 |
| LPCAT1 | 0.0032388 | 1 | 5726.63733 | 0.85450672 | 1.32E−77 | 2.37E−75 |
| CDC42EP3 | 2.24E−05 | 0.72758423 | 4467.29465 | 0.7812707 | 1.76E−76 | 3.14E−74 |
| AC018629.1 | 9.59E−05 | 1 | 903.447563 | −1.3794951 | 8.97E−75 | 1.58E−72 |
| SORL1 | 6.78E−05 | 1 | 988.68051 | 1.27220284 | 1.53E−74 | 2.65E−72 |
| OSMR | 0.00015276 | 1 | 4931.35512 | −0.7051025 | 2.13E−74 | 3.67E−72 |
| CEACAM6 | 0.00017613 | 1 | 50747.3301 | −0.5933493 | 2.71E−74 | 4.60E−72 |
| HLA-A | 0.00029708 | 1 | 5673.11535 | 0.73819311 | 5.30E−73 | 8.91E−71 |
| PTGS2 | 0.01194892 | 1 | 1523.2297 | −1.4177483 | 6.58E−73 | 1.10E−70 |
| PAM | 2.88E−05 | 0.93642669 | 4772.17662 | 0.76153132 | 1.20E−72 | 1.97E−70 |
| MUC16 | 0.00109623 | 1 | 215.51007 | 3.08002566 | 4.25E−72 | 6.92E−70 |
| ANXA9 | 0.00033775 | 1 | 725.651504 | 1.44508367 | 1.03E−71 | 1.66E−69 |
| TMEM9 | 0.001411 | 1 | 2254.86704 | 0.97352493 | 4.57E−71 | 7.29E−69 |
| ZBED2 | 0.00214193 | 1 | 1731.54976 | −1.0684979 | 2.69E−70 | 4.25E−68 |
| SYTL2 | 0.0005968 | 1 | 529.319714 | 1.64123256 | 4.08E−70 | 6.38E−68 |
| SPRED2 | 0.00017597 | 1 | 1725.24592 | −0.9608147 | 1.13E−69 | 1.75E−67 |
| TMC4 | 1.35E−05 | 0.43737491 | 911.089626 | 1.24391344 | 7.30E−68 | 1.12E−65 |
| ARHGAP26 | 0.00091159 | 1 | 1327.12309 | −1.0550711 | 9.09E−68 | 1.38E−65 |
| NDRG1 | 0.00015199 | 1 | 880.996068 | −1.2833518 | 4.42E−67 | 6.65E−65 |
| PERP | 0.00077532 | 1 | 10596.8507 | 0.66719501 | 4.91E−67 | 7.32E−65 |
| CHST3 | 3.63E−05 | 1 | 2778.99468 | 0.82019732 | 7.20E−67 | 1.06E−64 |
| MSLN | 3.47E−05 | 1 | 1467.68707 | 1.02966741 | 2.19E−66 | 3.21E−64 |
| MYL9 | 0.00020916 | 1 | 243.771973 | 2.5447763 | 2.47E−66 | 3.58E−64 |
| MUC1 | 0.00257123 | 1 | 1884.35597 | 0.92709104 | 8.27E−66 | 1.19E−63 |
| TCEA2 | 2.87E−05 | 0.93221876 | 1051.40424 | −1.1652374 | 1.42E−65 | 2.02E−63 |
| ATP2C2 | 0.00011705 | 1 | 1118.94521 | −1.3567168 | 1.47E−65 | 2.06E−63 |
| CEACAM5 | 0.00035144 | 1 | 6079.72512 | 0.83557684 | 1.47E−65 | 2.06E−63 |
| CDR2L | 0.00103961 | 1 | 2531.67872 | 0.8731038 | 1.64E−65 | 2.27E−63 |
| SPRED1 | 0.00052432 | 1 | 975.924837 | −1.4809136 | 9.14E−65 | 1.25E−62 |
| HLA-B | 0.00265149 | 1 | 775.543007 | 1.33117122 | 1.56E−64 | 2.12E−62 |
| DUSP4 | 0.00321568 | 1 | 9330.75068 | −0.804046 | 2.66E−64 | 3.59E−62 |
| ERBB2 | 7.68E−06 | 0.24983042 | 4222.09101 | 0.68415426 | 6.45E−64 | 8.63E−62 |
| PLAU | 0.00020463 | 1 | 1134.74283 | −1.1408778 | 8.52E−64 | 1.13E−61 |
| KIFC3 | 0.00066251 | 1 | 1989.92327 | −0.9491038 | 1.40E−63 | 1.85E−61 |
| LINC02747 | 0.00016857 | 1 | 673.057138 | 1.36041098 | 2.04E−63 | 2.66E−61 |
| KRT13 | 0.00048866 | 1 | 255.229659 | 2.57284187 | 3.03E−63 | 3.92E−61 |
| TMCO3 | 0.00038527 | 1 | 2767.65628 | 0.79180462 | 3.18E−63 | 4.08E−61 |
| AJUBA | 0.00221761 | 1 | 3544.1731 | 0.78501093 | 4.10E−63 | 5.21E−61 |
| TAGLN | 0.00574304 | 1 | 265.896857 | 2.48932775 | 7.35E−63 | 9.27E−61 |
| ADAM8 | 0.00110891 | 1 | 835.905821 | −1.4057316 | 7.49E−63 | 9.37E−61 |
| ABCC2 | 0.00048341 | 1 | 18669.9267 | 0.52036287 | 7.97E−63 | 9.89E−61 |
| COL4A4 | 8.67E−05 | 1 | 2717.08223 | 0.77523803 | 1.03E−62 | 1.27E−60 |
| CXCL8 | 0.01670099 | 1 | 333.393511 | −2.4594552 | 3.03E−62 | 3.70E−60 |
| GDF15 | 0.00056039 | 1 | 2936.98742 | −1.0313674 | 9.67E−62 | 1.17E−59 |
| IGF1R | 0.00014148 | 1 | 11282.251 | 0.63335923 | 7.84E−61 | 9.44E−59 |
| UPP1 | 0.00016947 | 1 | 1571.76344 | −0.9344184 | 4.59E−60 | 5.49E−58 |
| LAMC2 | 1.15E−05 | 0.37409342 | 10495.0761 | −0.5429762 | 7.96E−60 | 9.44E−58 |
| PDCD4 | 0.00019393 | 1 | 1676.4585 | 0.9103094 | 8.37E−60 | 9.85E−58 |
| GABARAPL1 | 0.00058154 | 1 | 3393.31641 | 0.70350051 | 1.10E−59 | 1.28E−57 |
| IGFN1 | 0.00324795 | 1 | 149.598057 | 4.79352017 | 1.82E−59 | 2.11E−57 |
| DKK1 | 0.00270343 | 1 | 812.405959 | −1.2568268 | 2.56E−59 | 2.95E−57 |
| NEBL | 0.0026302 | 1 | 1724.45715 | 0.93320838 | 5.19E−59 | 5.92E−57 |
| STEAP4 | 0.00035182 | 1 | 2457.59472 | 0.83318128 | 6.77E−59 | 7.68E−57 |
| SORT1 | 2.58E−05 | 0.83672433 | 2935.72537 | 0.73968925 | 7.06E−59 | 7.90E−57 |
| STAMBPL1 | 5.57E−06 | 0.18126123 | 549.193087 | −1.4411648 | 7.07E−59 | 7.90E−57 |
| GSN | 1.18E−05 | 0.38457884 | 1579.47074 | 0.89488375 | 1.15E−58 | 1.28E−56 |
| PDZK1IP1 | 6.57E−05 | 1 | 328.706192 | 1.99544615 | 1.52E−58 | 1.67E−56 |
| TMEM154 | 0.00249731 | 1 | 777.747094 | −1.2791293 | 3.77E−58 | 4.12E−56 |
| GPAT3 | 2.33E−05 | 0.75615942 | 2237.89659 | −0.7868243 | 8.82E−58 | 9.58E−56 |
| GSTP1 | 0.00364747 | 1 | 57010.1517 | 0.5438549 | 3.41E−57 | 3.68E−55 |
| GCLC | 5.98E−06 | 0.1944563 | 7687.1299 | 0.55492629 | 6.85E−57 | 7.34E−55 |
| PDLIM1 | 0.00022008 | 1 | 4691.47194 | 0.65294376 | 9.48E−57 | 1.01E−54 |
| L1CAM | 9.11E−05 | 1 | 464.843553 | 1.57846958 | 1.83E−56 | 1.93E−54 |
| DLX5 | 1.57E−05 | 0.51058295 | 572.759114 | 1.43107565 | 1.86E−56 | 1.96E−54 |
| SPTLC2 | 0.00025621 | 1 | 2466.05557 | 0.75105758 | 5.89E−56 | 6.15E−54 |
| PTPN12 | 0.00011393 | 1 | 5742.0788 | −0.6166816 | 6.07E−56 | 6.29E−54 |
| SLC7A5 | 0.00254907 | 1 | 50685.649 | 0.6093758 | 9.14E−56 | 9.41E−54 |
| HLA-C | 0.00452013 | 1 | 9465.92481 | 0.62962456 | 9.60E−56 | 9.82E−54 |
| TNS3 | 0.00155008 | 1 | 5758.63462 | 0.59439939 | 1.30E−55 | 1.32E−53 |
| CPS1 | 0.0029183 | 1 | 34413.9411 | 0.5129063 | 2.42E−55 | 2.44E−53 |
| TAPBP | 0.00244874 | 1 | 2374.95949 | 0.78592294 | 4.20E−55 | 4.21E−53 |
| CYP4F3 | 0.00018986 | 1 | 4408.25929 | 0.64795972 | 7.38E−55 | 7.35E−53 |
| MLX | 0.0014612 | 1 | 2355.38311 | −0.7666802 | 1.04E−54 | 1.03E−52 |
| DAG1 | 9.98E−06 | 0.32435212 | 8992.02951 | 0.52390644 | 1.81E−54 | 1.78E−52 |
| CLU | 0.00050155 | 1 | 1362.4774 | 0.94840943 | 6.76E−54 | 6.61E−52 |
| RARG | 0.00260323 | 1 | 1844.52389 | 0.830523 | 7.68E−54 | 7.46E−52 |
| ITGAV | 0.00027411 | 1 | 3368.75793 | 0.7120843 | 1.33E−53 | 1.28E−51 |
| SDC4 | 0.00054 | 1 | 5209.48536 | −0.6327169 | 2.24E−53 | 2.15E−51 |
| TJP1 | 0.00027734 | 1 | 16199.1273 | 0.49602509 | 2.85E−53 | 2.72E−51 |
| HSPB8 | 0.00113442 | 1 | 2564.6374 | 0.78409086 | 7.04E−53 | 6.67E−51 |
| LDB2 | 3.33E−06 | 0.10818142 | 127.073606 | −3.8852748 | 1.23E−52 | 1.16E−50 |
| IER3 | 0.00309915 | 1 | 497.370728 | −1.6462355 | 1.99E−52 | 1.87E−50 |
| CHP1 | 0.00038148 | 1 | 10984.9989 | 0.52901618 | 6.38E−52 | 5.94E−50 |
| CYFIP2 | 0.0001873 | 1 | 1065.97435 | 1.03626549 | 2.57E−51 | 2.38E−49 |
| DNMBP | 3.81E−05 | 1 | 1928.84442 | −0.7693442 | 2.74E−51 | 2.52E−49 |
| APOL6 | 0.00036125 | 1 | 612.796389 | 1.29801431 | 7.76E−51 | 7.10E−49 |
| ANTXR2 | 0.00038129 | 1 | 489.706403 | −1.4258488 | 3.11E−50 | 2.83E−48 |
| GJB2 | 7.61E−05 | 1 | 1459.19707 | −0.8959317 | 3.76E−50 | 3.40E−48 |
| ANK3 | 0.0001157 | 1 | 1197.88617 | 0.95346721 | 4.05E−50 | 3.64E−48 |
| STEAP1 | 0.00021094 | 1 | 1609.91919 | −0.8236973 | 4.33E−50 | 3.87E−48 |
| CYP26B1 | 1.60E−05 | 0.51837612 | 472.757697 | −1.4589799 | 7.75E−50 | 6.89E−48 |
| SPRY2 | 0.00675695 | 1 | 366.818303 | −1.7733421 | 1.22E−49 | 1.08E−47 |
| PLEKHG2 | 0.00146814 | 1 | 759.731846 | −1.1422098 | 1.31E−49 | 1.15E−47 |
| PLEK2 | 4.72E−07 | 0.01537011 | 1467.53429 | −0.8325903 | 1.97E−49 | 1.72E−47 |
| TNC | 1.66E−06 | 0.05414619 | 1058.57444 | −0.9587399 | 2.48E−49 | 2.15E−47 |
| LRP1 | 8.12E−05 | 1 | 881.787655 | 1.08765124 | 4.68E−49 | 4.04E−47 |
| CXCL16 | 0.00145453 | 1 | 2339.96465 | 0.7538961 | 1.36E−48 | 1.17E−46 |
| TFPI2 | 0.00115412 | 1 | 8182.0829 | −0.6422236 | 2.07E−48 | 1.77E−46 |
| NTN4 | 2.74E−05 | 0.88921529 | 1619.28363 | 0.83040033 | 2.41E−48 | 2.05E−46 |
| SAMD11 | 6.19E−05 | 1 | 311.526873 | 1.90258851 | 3.11E−48 | 2.63E−46 |
| AHNAK2 | 0.00348511 | 1 | 2027.68314 | 0.93868448 | 4.23E−48 | 3.56E−46 |
| PFKFB3 | 0.00362943 | 1 | 3127.44203 | 0.72299361 | 6.40E−48 | 5.35E−46 |
| TMCC3 | 0.00048536 | 1 | 1038.4045 | 1.06137286 | 1.22E−47 | 1.02E−45 |
| FECH | 0.00021721 | 1 | 6337.83759 | 0.52077828 | 1.74E−47 | 1.44E−45 |
| B3GNT3 | 0.00094707 | 1 | 1794.34608 | −0.7710014 | 2.69E−47 | 2.22E−45 |
| SEMA3A | 0.00016846 | 1 | 2292.61629 | −0.8103263 | 3.06E−47 | 2.50E−45 |
| ATP9A | 0.00076562 | 1 | 2612.1885 | 0.67937036 | 4.31E−47 | 3.51E−45 |
| DSTN | 2.96E−05 | 0.96192584 | 11608.9828 | 0.48653216 | 1.07E−46 | 8.71E−45 |
| NR3C1 | 0.00033099 | 1 | 8371.40834 | 0.52376249 | 1.23E−45 | 9.93E−44 |
| AKR1C3 | 0.00132813 | 1 | 18417.4488 | 0.53907572 | 3.45E−45 | 2.77E−43 |
| PDXK | 0.00110446 | 1 | 8243.01023 | 0.55455992 | 4.54E−45 | 3.63E−43 |
| FLG | 2.12E−05 | 0.68891356 | 210.97627 | 2.16114219 | 4.90E−45 | 3.89E−43 |
| MYO5B | 0.00042811 | 1 | 1946.82226 | 0.7159678 | 1.10E−44 | 8.72E−43 |
| ABHD4 | 0.00100218 | 1 | 5108.83536 | 0.64636051 | 1.47E−44 | 1.16E−42 |
| LDHA | 2.21E−05 | 0.71736966 | 49133.5724 | −0.3996372 | 2.91E−44 | 2.28E−42 |
| MAB21L4 | 2.33E−05 | 0.75799901 | 481.262445 | 1.32888236 | 1.79E−43 | 1.39E−41 |
| ITPRIP | 0.00073851 | 1 | 1296.21607 | −0.8597673 | 1.96E−43 | 1.52E−41 |
| PSME1 | 0.0001915 | 1 | 4280.73344 | 0.55372272 | 2.51E−43 | 1.93E−41 |
| HSPG2 | 0.00023439 | 1 | 12070.0578 | 0.54908104 | 3.28E−43 | 2.51E−41 |
| MCTP1 | 0.00068683 | 1 | 256.178695 | −1.8212772 | 4.14E−43 | 3.16E−41 |
| TP53 | 4.81E−05 | 1 | 1394.33094 | 0.80792341 | 6.04E−43 | 4.59E−41 |
| LINC01133 | 0.00445586 | 1 | 150.738338 | 2.69732491 | 1.01E−42 | 7.63E−41 |
| AC026785.3 | 9.80E−05 | 1 | 1126.72513 | 0.88624894 | 2.63E−42 | 1.97E−40 |
| IVL | 0.01395367 | 1 | 628.9418 | 1.31218302 | 4.04E−42 | 3.02E−40 |
| SLC22A5 | 0.00554639 | 1 | 2859.9722 | 0.65529322 | 4.35E−42 | 3.24E−40 |
| TTYH3 | 0.00688574 | 1 | 804.774501 | 1.11472738 | 5.38E−42 | 3.99E−40 |
| MYO5C | 1.08E−05 | 0.35040981 | 5747.07353 | 0.49771968 | 1.35E−41 | 9.97E−40 |
| HEG1 | 5.51E−05 | 1 | 650.323045 | 1.14713433 | 1.57E−41 | 1.15E−39 |
| FAM83A | 0.0001224 | 1 | 4560.27452 | −0.55582 | 1.59E−41 | 1.16E−39 |
| FTL | 0.00129403 | 1 | 128780.067 | 0.48981841 | 1.99E−41 | 1.45E−39 |
| EMP2 | 1.47E−05 | 0.4777324 | 1361.01576 | 0.79914336 | 4.60E−41 | 3.33E−39 |
| GAA | 6.76E−05 | 1 | 5322.17112 | 0.53803828 | 5.42E−41 | 3.91E−39 |
| AKAP13 | 0.00023644 | 1 | 4519.49177 | 0.54971039 | 9.10E−41 | 6.53E−39 |
| CPE | 0.00016125 | 1 | 2653.51285 | 0.65423375 | 1.79E−40 | 1.28E−38 |
| RHOB | 0.00620002 | 1 | 3449.71945 | 0.66582005 | 2.01E−40 | 1.43E−38 |
| GPX2 | 0.00054105 | 1 | 11177.5754 | 0.53370908 | 6.21E−40 | 4.40E−38 |
| HR | 0.00018029 | 1 | 532.558973 | 1.3440989 | 6.66E−40 | 4.69E−38 |
| TSPAN1 | 0.00067945 | 1 | 6242.90956 | 0.60248435 | 7.21E−40 | 5.06E−38 |
| LIPG | 0.00156587 | 1 | 242.67803 | −1.7726065 | 8.62E−40 | 6.02E−38 |
| BAG1 | 0.00043999 | 1 | 4466.71708 | 0.56927488 | 1.14E−39 | 7.94E−38 |
| KIAA0040 | 0.00718484 | 1 | 611.085552 | −1.1691088 | 1.40E−39 | 9.72E−38 |
| SMIM14 | 9.34E−05 | 1 | 2391.78249 | 0.65799497 | 1.57E−39 | 1.08E−37 |
| BMP1 | 2.47E−08 | 0.00080383 | 1733.52115 | 0.68794865 | 1.60E−39 | 1.10E−37 |
| ATP2A3 | 3.09E−06 | 0.10052694 | 288.390186 | 1.59255257 | 2.61E−39 | 1.78E−37 |
| ROCK2 | 0.00028268 | 1 | 4453.30481 | 0.57392139 | 3.55E−39 | 2.41E−37 |
| AP1S1 | 0.00248316 | 1 | 7931.89828 | −0.5712316 | 7.89E−39 | 5.34E−37 |
| CELSR1 | 0.00223981 | 1 | 3648.9521 | 0.59595624 | 1.06E−38 | 7.18E−37 |
| FAM13B | 0.00078488 | 1 | 1222.16899 | 0.84452327 | 1.25E−38 | 8.40E−37 |
| UBALD2 | 0.00081508 | 1 | 461.716091 | −1.2698454 | 1.57E−38 | 1.05E−36 |
| GLCCI1 | 0.01254156 | 1 | 1042.72042 | 0.99687697 | 1.89E−38 | 1.26E−36 |
| CTNNAL1 | 0.0003551 | 1 | 4500.89747 | −0.5236975 | 2.03E−38 | 1.35E−36 |
| ULK1 | 0.00086018 | 1 | 923.713625 | 0.93915379 | 2.34E−38 | 1.54E−36 |
| TMEM59 | 0.0001069 | 1 | 3484.39568 | 0.58830646 | 2.93E−38 | 1.93E−36 |
| CASC19 | 0.00017972 | 1 | 109.46414 | −3.0195757 | 7.01E−38 | 4.59E−36 |
| ERAP2 | 0.00166374 | 1 | 1392.67509 | 0.74820464 | 7.92E−38 | 5.16E−36 |
| HTRA3 | 0.00696357 | 1 | 2063.99401 | 0.73409915 | 1.22E−37 | 7.89E−36 |
| ETS2 | 0.00169091 | 1 | 3742.889 | 0.55303221 | 1.53E−37 | 9.89E−36 |
| HMGA1 | 0.0001177 | 1 | 15412.3899 | −0.439326 | 2.48E−37 | 1.60E−35 |
| NECTIN4 | 0.00019571 | 1 | 2152.98703 | 0.63902607 | 2.79E−37 | 1.79E−35 |
| PPARG | 0.00023642 | 1 | 317.397211 | −1.5065372 | 5.08E−37 | 3.25E−35 |
| LYAR | 0.00046527 | 1 | 2301.13144 | −0.6264429 | 1.12E−36 | 7.13E−35 |
| RAPGEFL1 | 0.0018284 | 1 | 504.13284 | 1.16754694 | 1.19E−36 | 7.53E−35 |
| TGM1 | 7.60E−05 | 1 | 132.960053 | 2.55802153 | 1.32E−36 | 8.32E−35 |
| SH2D3A | 1.31E−05 | 0.42551905 | 2471.37632 | −0.5939737 | 1.95E−36 | 1.22E−34 |
| MGLL | 0.00740334 | 1 | 4602.6396 | 0.55996276 | 2.64E−36 | 1.65E−34 |
| SLC29A3 | 0.00168961 | 1 | 457.10405 | 1.23757537 | 2.86E−36 | 1.78E−34 |
| DDC | 0.00032137 | 1 | 372.373299 | 1.35521976 | 3.28E−36 | 2.04E−34 |
| ASS1 | 0.00098279 | 1 | 1701.49444 | 0.74019552 | 4.11E−36 | 2.53E−34 |
| TSPAN15 | 7.08E−05 | 1 | 1756.26082 | 0.665523 | 4.11E−36 | 2.53E−34 |
| EPHX1 | 0.0023676 | 1 | 6776.41049 | 0.55652622 | 4.19E−36 | 2.57E−34 |
| GALNT7 | 0.00130334 | 1 | 3006.0981 | 0.63284585 | 4.40E−36 | 2.69E−34 |
| TGFA | 0.00020513 | 1 | 2594.87576 | −0.593 | 5.18E−36 | 3.15E−34 |
| KIAA0319 | 0.00051964 | 1 | 1271.5758 | 0.85542222 | 6.37E−36 | 3.86E−34 |
| MVP | 0.00019065 | 1 | 2977.8674 | 0.56460697 | 6.96E−36 | 4.20E−34 |
| DTX4 | 5.36E−05 | 1 | 705.80458 | 0.99779244 | 8.01E−36 | 4.82E−34 |
| LAMB3 | 0.00489642 | 1 | 30229.523 | 0.39703225 | 1.16E−35 | 6.94E−34 |
| AKR1B1 | 0.00402719 | 1 | 8196.87026 | 0.48777358 | 1.34E−35 | 8.01E−34 |
| IDH1 | 0.0015817 | 1 | 7886.97662 | 0.50137197 | 1.60E−35 | 9.50E−34 |
| DENND3 | 0.00059097 | 1 | 916.770039 | 0.90700207 | 1.68E−35 | 9.97E−34 |
| FSCN1 | 0.0070627 | 1 | 13732.6648 | −0.583958 | 1.70E−35 | 1.01E−33 |
| MT2A | 0.007981 | 1 | 1184.68551 | −0.8421293 | 1.94E−35 | 1.14E−33 |
| PTPRF | 0.00110598 | 1 | 11171.4233 | 0.44527664 | 2.18E−35 | 1.28E−33 |
| BAMBI | 0.00039011 | 1 | 1412.94857 | 0.7444242 | 2.64E−35 | 1.54E−33 |
| RNF213 | 0.00093223 | 1 | 9205.67764 | 0.5969818 | 3.15E−35 | 1.83E−33 |
| TNFAIP2 | 8.23E−05 | 1 | 2783.41551 | −0.5845238 | 3.96E−35 | 2.30E−33 |
| MAN2B2 | 0.00038552 | 1 | 2811.13552 | 0.6163519 | 4.12E−35 | 2.38E−33 |
| TXN | 0.00382415 | 1 | 32570.4821 | 0.43388307 | 4.60E−35 | 2.64E−33 |
| SLC45A3 | 0.00033877 | 1 | 459.28603 | −1.2130333 | 5.12E−35 | 2.94E−33 |
| LGALS9B | 0.00207484 | 1 | 146.912 | −2.2039841 | 6.42E−35 | 3.67E−33 |
| LBH | 3.68E−05 | 1 | 345.276906 | 1.33924144 | 8.25E−35 | 4.70E−33 |
| IFI27 | 0.0105661 | 1 | 148.257594 | 2.54964375 | 9.99E−35 | 5.66E−33 |
| ACSL3 | 0.00062512 | 1 | 5722.21131 | −0.5191491 | 1.03E−34 | 5.80E−33 |
| FERMT1 | 0.00096634 | 1 | 5837.97324 | −0.4676803 | 1.12E−34 | 6.28E−33 |
| SCARB2 | 0.00013565 | 1 | 4682.72109 | 0.47461194 | 1.22E−34 | 6.85E−33 |
| OSBPL7 | 0.00025621 | 1 | 1246.44262 | 0.83380442 | 1.70E−34 | 9.53E−33 |
| APOL2 | 0.00048427 | 1 | 1257.27391 | 0.73616199 | 1.73E−34 | 9.62E−33 |
| ANXA4 | 0.0002435 | 1 | 2114.88847 | 0.61603102 | 2.89E−34 | 1.60E−32 |
| GLP2R | 0.00504016 | 1 | 448.727443 | 1.20459399 | 4.41E−34 | 2.44E−32 |
| SLC7A2 | 0.0019286 | 1 | 7699.4534 | −0.5435815 | 6.57E−34 | 3.62E−32 |
| MUC13 | 0.00080955 | 1 | 7935.76589 | 0.50037862 | 1.44E−33 | 7.92E−32 |
| LFNG | 0.00840147 | 1 | 409.362489 | 1.28396852 | 2.10E−33 | 1.15E−31 |
| LTBP2 | 0.00236648 | 1 | 467.980699 | −1.1390738 | 2.31E−33 | 1.26E−31 |
| CTSS | 8.57E−05 | 1 | 99.3231412 | 2.8181082 | 2.65E−33 | 1.44E−31 |
| ARHGEF2 | 0.00324882 | 1 | 1953.14351 | −0.6661443 | 3.17E−33 | 1.72E−31 |
| RASSF2 | 0.00251335 | 1 | 238.77456 | 1.67672916 | 5.85E−33 | 3.16E−31 |
| CCDC80 | 0.00044509 | 1 | 429.798174 | 1.25752553 | 6.21E−33 | 3.34E−31 |
| ATOH8 | 0.00168236 | 1 | 114.113128 | 2.53025056 | 6.32E−33 | 3.38E−31 |
| FST | 3.15E−06 | 0.10247696 | 101.893541 | −2.7272827 | 7.41E−33 | 3.94E−31 |
| NUCB1 | 0.00385946 | 1 | 1743.93174 | 0.67811355 | 8.61E−33 | 4.57E−31 |
| TACSTD2 | 0.01210933 | 1 | 11485.2967 | 0.48876272 | 9.33E−33 | 4.93E−31 |
| IL1A | 0.01425101 | 1 | 636.331602 | −1.1066583 | 1.23E−32 | 6.48E−31 |
| WDR66 | 0.00012021 | 1 | 300.283869 | 1.45513708 | 1.43E−32 | 7.48E−31 |
| INSL4 | 0.00144849 | 1 | 965.572517 | 0.8042675 | 1.43E−32 | 7.50E−31 |
| TAPBPL | 0.00275045 | 1 | 225.135999 | 1.68284679 | 1.71E−32 | 8.94E−31 |
| PLAAT3 | 1.90E−05 | 0.61817669 | 643.758815 | 0.94592238 | 1.82E−32 | 9.45E−31 |
| GPR153 | 0.00224043 | 1 | 1185.36086 | −0.7566609 | 2.14E−32 | 1.11E−30 |
| RASGRP1 | 0.00208581 | 1 | 380.923076 | 1.29270404 | 2.14E−32 | 1.11E−30 |
| PPP1R13L | 0.0060425 | 1 | 2241.58969 | 0.60956733 | 2.20E−32 | 1.13E−30 |
| CTSD | 0.00037992 | 1 | 7240.77578 | 0.45295604 | 2.37E−32 | 1.21E−30 |
| ABCC3 | 0.00075074 | 1 | 5756.36795 | 0.50402182 | 2.46E−32 | 1.26E−30 |
| LINC00973 | 0.00176267 | 1 | 119.353129 | −2.4797863 | 2.71E−32 | 1.38E−30 |
| RHOBTB3 | 0.0042867 | 1 | 8189.35382 | 0.51002054 | 6.05E−32 | 3.07E−30 |
| EPB41L4A | 0.00701572 | 1 | 104.42337 | 2.71408632 | 6.13E−32 | 3.11E−30 |
| HSPB1 | 0.00753072 | 1 | 2809.5639 | 0.64764538 | 7.21E−32 | 3.64E−30 |
| DDR1 | 0.00193114 | 1 | 4610.85875 | 0.48069813 | 9.14E−32 | 4.60E−30 |
| GNG11 | 0.00338322 | 1 | 1888.74088 | −0.6286459 | 1.08E−31 | 5.42E−30 |
| CCNB1 | 0.0001415 | 1 | 9700.30614 | −0.3790869 | 1.41E−31 | 7.04E−30 |
| DGCR2 | 0.00128764 | 1 | 2784.14918 | 0.52821256 | 1.55E−31 | 7.70E−30 |
| YRDC | 0.00116984 | 1 | 4765.53241 | −0.5477593 | 1.76E−31 | 8.75E−30 |
| STOM | 0.00066076 | 1 | 3554.61236 | 0.50975248 | 1.98E−31 | 9.81E−30 |
| 4-Mar | 0.00112297 | 1 | 78.6259456 | −3.443853 | 2.22E−31 | 1.10E−29 |
| PHC2 | 0.00075211 | 1 | 2016.37132 | −0.5953769 | 3.34E−31 | 1.65E−29 |
| PSMB8 | 0.0012708 | 1 | 618.108657 | 0.97970281 | 4.29E−31 | 2.10E−29 |
| SLC3A2 | 0.00219797 | 1 | 20935.2002 | 0.3717552 | 4.46E−31 | 2.18E−29 |
| LUCAT1 | 7.15E−05 | 1 | 1350.75624 | −0.7004399 | 4.54E−31 | 2.21E−29 |
| PHGDH | 0.00155912 | 1 | 2148.72956 | 0.64694739 | 4.74E−31 | 2.30E−29 |
| CAPN2 | 9.66E−05 | 1 | 20026.1655 | −0.3415908 | 9.35E−31 | 4.53E−29 |
| PLXNB1 | 1.80E−05 | 0.58487659 | 1389.9459 | 0.67170212 | 1.21E−30 | 5.83E−29 |
| C6orf132 | 2.69E−05 | 0.87215583 | 2057.58571 | 0.57037159 | 1.55E−30 | 7.46E−29 |
| TBC1D2 | 0.00081901 | 1 | 3734.12926 | 0.52331434 | 1.58E−30 | 7.56E−29 |
| LGALS3 | 0.00181003 | 1 | 5737.11965 | 0.4258686 | 1.75E−30 | 8.36E−29 |
| RBMS2 | 0.00016948 | 1 | 3688.54544 | −0.4744805 | 1.75E−30 | 8.36E−29 |
| SLCO4A1 | 0.00020345 | 1 | 2156.05175 | −0.5873816 | 2.97E−30 | 1.41E−28 |
| FRMD6 | 0.00043048 | 1 | 719.17335 | −0.8960305 | 4.23E−30 | 2.01E−28 |
| TIMP2 | 4.13E−06 | 0.13440946 | 2803.97297 | 0.50559447 | 5.81E−30 | 2.75E−28 |
| S100A16 | 0.00065129 | 1 | 17296.4179 | −0.3924539 | 7.90E−30 | 3.73E−28 |
| JCAD | 0.00010188 | 1 | 195.937052 | −1.7491039 | 8.70E−30 | 4.09E−28 |
| ANKRD2 | 0.00082812 | 1 | 285.486565 | 1.39233868 | 9.97E−30 | 4.67E−28 |
| F3 | 0.00372721 | 1 | 8588.75637 | −0.4240957 | 9.99E−30 | 4.67E−28 |
| COBL | 0.00213045 | 1 | 3703.15034 | 0.50914973 | 1.20E−29 | 5.59E−28 |
| CREG2 | 0.00517195 | 1 | 1357.22102 | 0.75281574 | 1.29E−29 | 5.99E−28 |
| SAMD9L | 0.00681058 | 1 | 661.119504 | 0.93408901 | 1.79E−29 | 8.28E−28 |
| FA2H | 0.00141411 | 1 | 2807.38153 | 0.51167835 | 2.13E−29 | 9.84E−28 |
| IRS1 | 0.01139683 | 1 | 1069.90111 | −0.8204225 | 2.96E−29 | 1.36E−27 |
| MB | 0.00030499 | 1 | 88.4166696 | 2.75165521 | 3.55E−29 | 1.63E−27 |
| SCD | 0.00128683 | 1 | 36350.985 | −0.3682506 | 4.45E−29 | 2.04E−27 |
| CRYBG2 | 2.00E−05 | 0.65055142 | 2090.66413 | 0.55544339 | 4.99E−29 | 2.28E−27 |
| CA2 | 0.00515955 | 1 | 447.263575 | −1.1522434 | 5.26E−29 | 2.39E−27 |
| ELF3 | 0.00168265 | 1 | 2513.67937 | 0.60669059 | 5.33E−29 | 2.42E−27 |
| ID3 | 0.00079543 | 1 | 2978.10818 | 0.54772061 | 7.21E−29 | 3.26E−27 |
| IGFBP2 | 0.01912849 | 1 | 1601.72554 | 0.7466293 | 7.40E−29 | 3.34E−27 |
| CADM1 | 6.56E−05 | 1 | 429.242391 | 1.11706812 | 7.79E−29 | 3.50E−27 |
| TOR4A | 0.0054859 | 1 | 1678.58393 | −0.6945846 | 1.34E−28 | 5.99E−27 |
| UCHL1 | 0.00429266 | 1 | 23722.0875 | 0.38207775 | 1.50E−28 | 6.70E−27 |
| TOP1 | 0.00058137 | 1 | 5398.78174 | −0.4392934 | 1.59E−28 | 7.07E−27 |
| NADSYN1 | 8.73E−05 | 1 | 2254.05117 | 0.55736036 | 1.68E−28 | 7.47E−27 |
| SPHK1 | 2.14E−05 | 0.69394993 | 714.253415 | −0.8527842 | 2.14E−28 | 9.49E−27 |
| KIRREL1 | 7.27E−06 | 0.23631408 | 1816.98598 | −0.5739926 | 2.69E−28 | 1.19E−26 |
| OPTN | 0.00736135 | 1 | 1429.41794 | 0.68228949 | 3.53E−28 | 1.55E−26 |
| MEGF9 | 0.00052224 | 1 | 1749.15522 | 0.63568142 | 4.38E−28 | 1.93E−26 |
| LINC00963 | 2.26E−05 | 0.73439301 | 929.588744 | 0.75218835 | 5.36E−28 | 2.35E−26 |
| OLMALINC | 0.00460039 | 1 | 391.60105 | −1.2090492 | 6.17E−28 | 2.70E−26 |
| PITPNC1 | 0.00017679 | 1 | 365.45634 | −1.1933263 | 6.35E−28 | 2.77E−26 |
| ARHGAP23 | 0.00012638 | 1 | 793.649362 | 0.84116851 | 6.99E−28 | 3.04E−26 |
| ECT2 | 0.00142761 | 1 | 5213.77366 | −0.496263 | 7.54E−28 | 3.27E−26 |
| NTNG1 | 0.00124354 | 1 | 469.432779 | −1.0304336 | 7.85E−28 | 3.39E−26 |
| COL8A1 | 4.74E−06 | 0.1542292 | 212.353637 | −1.519858 | 1.02E−27 | 4.40E−26 |
| SH3BGRL2 | 0.00102012 | 1 | 675.25948 | 0.90529999 | 1.04E−27 | 4.49E−26 |
| SERINC5 | 0.00028497 | 1 | 1690.68323 | 0.5741228 | 1.31E−27 | 5.60E−26 |
| BCL2L1 | 0.00031002 | 1 | 3854.63865 | −0.495396 | 1.52E−27 | 6.51E−26 |
| PRKCA | 0.00018851 | 1 | 1182.08649 | −0.7153661 | 1.55E−27 | 6.60E−26 |
| FN1 | 3.51E−05 | 1 | 399.708888 | 1.10105976 | 1.88E−27 | 8.00E−26 |
| LETM2 | 0.00081012 | 1 | 200.286708 | −1.5763303 | 2.09E−27 | 8.87E−26 |
| MTURN | 0.00192826 | 1 | 743.382474 | 0.83480571 | 2.10E−27 | 8.90E−26 |
| GLUL | 0.00029933 | 1 | 5474.43578 | 0.43797522 | 2.14E−27 | 9.01E−26 |
| PALLD | 0.00218255 | 1 | 5963.49577 | 0.42297754 | 2.17E−27 | 9.15E−26 |
| C1orf116 | 9.80E−05 | 1 | 5481.40267 | 0.42032609 | 2.31E−27 | 9.67E−26 |
| SPTAN1 | 0.00350648 | 1 | 14814.2551 | 0.3884215 | 2.41E−27 | 1.01E−25 |
| TPRA1 | 0.00046616 | 1 | 1577.09815 | 0.61483375 | 2.69E−27 | 1.12E−25 |
| NAV3 | 0.00026283 | 1 | 741.550782 | −0.8284591 | 3.04E−27 | 1.27E−25 |
| MXD4 | 0.00095458 | 1 | 852.925392 | 0.85739806 | 3.19E−27 | 1.32E−25 |
| ITM2B | 0.00042415 | 1 | 2816.73579 | 0.52284592 | 3.25E−27 | 1.35E−25 |
| TUFT1 | 0.00022688 | 1 | 2768.59604 | 0.52666129 | 3.39E−27 | 1.40E−25 |
| KIAA1522 | 1.12E−06 | 0.03658561 | 8157.74184 | 0.35841129 | 4.11E−27 | 1.69E−25 |
| CDC42BPB | 0.00094876 | 1 | 5404.42436 | 0.39398874 | 8.68E−27 | 3.56E−25 |
| TTC9 | 0.00019332 | 1 | 1969.63448 | 0.56282456 | 9.21E−27 | 3.77E−25 |
| G6PC3 | 0.00175218 | 1 | 2833.36303 | 0.54184611 | 1.42E−26 | 5.81E−25 |
| SERPINE2 | 0.00250509 | 1 | 1042.88816 | −0.7137194 | 1.63E−26 | 6.63E−25 |
| TSPAN8 | 0.00029998 | 1 | 2528.09974 | 0.52879651 | 1.88E−26 | 7.63E−25 |
| PRKCD | 0.00013517 | 1 | 1942.29527 | 0.55821863 | 2.01E−26 | 8.14E−25 |
| RHOU | 0.00054672 | 1 | 656.23402 | 0.92493946 | 2.32E−26 | 9.37E−25 |
| LAMP2 | 0.00224305 | 1 | 2401.12184 | 0.5151685 | 2.33E−26 | 9.40E−25 |
| B4GALT5 | 0.00026489 | 1 | 3831.19271 | 0.4635588 | 2.53E−26 | 1.02E−24 |
| ARHGAP21 | 0.00173026 | 1 | 7826.95905 | 0.41483825 | 2.73E−26 | 1.09E−24 |
| AFF1 | 0.01002454 | 1 | 2516.03057 | 0.5794978 | 2.79E−26 | 1.12E−24 |
| BNIP2 | 0.00178293 | 1 | 2747.96287 | −0.6136342 | 3.15E−26 | 1.26E−24 |
| CBX6 | 0.00613184 | 1 | 2662.78153 | 0.53151117 | 3.32E−26 | 1.32E−24 |
| FKBP4 | 0.00035124 | 1 | 9900.03085 | −0.3712906 | 3.41E−26 | 1.35E−24 |
| TNFSF15 | 0.00048051 | 1 | 1233.83345 | −0.6969947 | 3.50E−26 | 1.39E−24 |
| CXXC5 | 0.00086527 | 1 | 2163.71015 | 0.57468275 | 3.94E−26 | 1.56E−24 |
| CTSA | 6.34E−05 | 1 | 3108.12706 | 0.47232747 | 4.04E−26 | 1.59E−24 |
| SLC16A14 | 0.0013054 | 1 | 2365.21099 | −0.5843365 | 4.15E−26 | 1.63E−24 |
| ADGRE5 | 0.00087295 | 1 | 1628.39587 | −0.6242815 | 5.04E−26 | 1.98E−24 |
| HTT | 0.00172956 | 1 | 5168.13176 | 0.44299926 | 5.11E−26 | 2.00E−24 |
| LINC00460 | 0.00514872 | 1 | 77.2643821 | −3.0459894 | 5.20E−26 | 2.03E−24 |
| RAC2 | 0.00013811 | 1 | 451.673326 | −1.029554 | 7.43E−26 | 2.89E−24 |
| UBL3 | 0.00331894 | 1 | 1545.67348 | 0.60238215 | 8.42E−26 | 3.27E−24 |
| CEMIP | 0.01064982 | 1 | 279.941867 | −1.3549858 | 9.15E−26 | 3.54E−24 |
| FRK | 0.00964334 | 1 | 1427.30408 | 0.71119057 | 9.20E−26 | 3.55E−24 |
| IL4R | 7.57E−06 | 0.24603889 | 938.389718 | −0.7091687 | 9.75E−26 | 3.76E−24 |
| INSYN2B | 0.00125933 | 1 | 87.2271457 | −2.5315344 | 1.01E−25 | 3.89E−24 |
| RNF145 | 0.00050111 | 1 | 3886.6668 | 0.47630236 | 1.09E−25 | 4.18E−24 |
| FOSL2 | 0.0029313 | 1 | 8644.87585 | 0.39675454 | 1.13E−25 | 4.32E−24 |
| CELSR3 | 0.00859268 | 1 | 980.895702 | 0.75444477 | 1.49E−25 | 5.69E−24 |
| DSP | 0.00266568 | 1 | 12040.5675 | 0.36257438 | 1.86E−25 | 7.09E−24 |
| TPM3 | 0.00013831 | 1 | 22938.3203 | −0.3260545 | 1.96E−25 | 7.45E−24 |
| IGSF9 | 0.00042031 | 1 | 1752.60716 | 0.55721315 | 2.19E−25 | 8.30E−24 |
| MFSD2A | 0.0003058 | 1 | 275.474381 | −1.2810912 | 2.98E−25 | 1.12E−23 |
| SLC41A2 | 0.00040792 | 1 | 814.420363 | 0.80967447 | 3.04E−25 | 1.15E−23 |
| CHST11 | 2.04E−05 | 0.66406411 | 1332.74535 | −0.6112268 | 3.87E−25 | 1.45E−23 |
| FLG-AS1 | 0.0007007 | 1 | 138.746966 | 1.90934715 | 3.89E−25 | 1.46E−23 |
| FIBP | 0.00026931 | 1 | 1893.47156 | 0.55621851 | 3.91E−25 | 1.46E−23 |
| VWA7 | 0.00344431 | 1 | 189.905135 | 1.56642034 | 4.85E−25 | 1.81E−23 |
| KIF1C | 0.00046623 | 1 | 11102.9296 | 0.37228874 | 5.07E−25 | 1.89E−23 |
| BCCIP | 1.86E−05 | 0.60435752 | 4219.14216 | −0.4099355 | 6.19E−25 | 2.30E−23 |
| LIPA | 0.00025929 | 1 | 3168.6079 | 0.48171327 | 7.59E−25 | 2.81E−23 |
| CDKN1C | 0.02694544 | 1 | 188.069173 | 1.74006858 | 1.10E−24 | 4.05E−23 |
| DLC1 | 0.00057438 | 1 | 138.812151 | −2.0246273 | 1.63E−24 | 6.00E−23 |
| HIF1A | 0.00498644 | 1 | 8532.21546 | −0.3687275 | 1.87E−24 | 6.88E−23 |
| ANKRD22 | 0.00025182 | 1 | 712.698384 | −0.815348 | 1.93E−24 | 7.08E−23 |
| DUSP5 | 1.84E−05 | 0.59764989 | 327.094879 | −1.1272933 | 2.20E−24 | 8.06E−23 |
| PCDH7 | 0.00032599 | 1 | 1277.47201 | 0.61656217 | 2.84E−24 | 1.04E−22 |
| AL590004.3 | 0.00047743 | 1 | 63.5562844 | 3.15801243 | 2.97E−24 | 1.08E−22 |
| ABCG2 | 8.04E−06 | 0.26136567 | 1535.32776 | 0.56188016 | 3.15E−24 | 1.15E−22 |
| DHRS9 | 0.00039247 | 1 | 1157.17429 | −0.6530987 | 3.26E−24 | 1.18E−22 |
| LINC01484 | 0.00503815 | 1 | 1433.31138 | 0.63262618 | 3.26E−24 | 1.18E−22 |
| CLSTN1 | 0.00090002 | 1 | 5091.71273 | 0.39999036 | 3.48E−24 | 1.26E−22 |
| PGRMC2 | 0.0002335 | 1 | 2138.29661 | 0.52807448 | 3.66E−24 | 1.32E−22 |
| MRGBP | 0.00050868 | 1 | 1854.2955 | −0.5776883 | 3.75E−24 | 1.35E−22 |
| ERV3-1 | 9.17E−07 | 0.02985038 | 287.136085 | 1.18873008 | 3.87E−24 | 1.39E−22 |
| TMPRSS3 | 0.00078582 | 1 | 609.746235 | 0.8421827 | 4.07E−24 | 1.46E−22 |
| ACTG1 | 0.00277727 | 1 | 31033.8812 | −0.2968423 | 4.13E−24 | 1.47E−22 |
| RDX | 0.00100271 | 1 | 21344.6765 | 0.32214718 | 4.45E−24 | 1.59E−22 |
| PTPRS | 0.00873385 | 1 | 3205.20399 | 0.51189925 | 4.70E−24 | 1.67E−22 |
| MYO15B | 0.00041972 | 1 | 403.734765 | 1.02038064 | 5.23E−24 | 1.85E−22 |
| CHD3 | 0.00013523 | 1 | 4239.99323 | 0.40542521 | 7.78E−24 | 2.75E−22 |
| BCAR3 | 0.0005427 | 1 | 2511.30071 | −0.5036037 | 7.98E−24 | 2.82E−22 |
| FHOD3 | 0.00077531 | 1 | 231.071018 | −1.3859499 | 8.49E−24 | 2.99E−22 |
| EFNA1 | 8.21E−05 | 1 | 1342.65888 | 0.59453888 | 8.52E−24 | 3.00E−22 |
| PRDM1 | 0.00221508 | 1 | 454.042852 | −0.9834683 | 9.06E−24 | 3.18E−22 |
| VSIR | 0.00011038 | 1 | 548.681261 | 0.89524827 | 1.00E−23 | 3.50E−22 |
| ITGA5 | 6.88E−05 | 1 | 1115.90359 | −0.6391797 | 1.18E−23 | 4.11E−22 |
| LAMP1 | 0.01825124 | 1 | 10049.1505 | 0.42742713 | 1.26E−23 | 4.37E−22 |
| SLC6A14 | 0.00287978 | 1 | 1153.45434 | −0.7931223 | 1.25E−23 | 4.37E−22 |
| FKBP14 | 3.53E−05 | 1 | 661.526767 | −0.7891798 | 1.37E−23 | 4.77E−22 |
| ARHGEF17 | 0.00075948 | 1 | 1620.00308 | 0.57008807 | 1.80E−23 | 6.22E−22 |
| AF121898.1 | 0.00635155 | 1 | 86.4426566 | −2.4105227 | 1.94E−23 | 6.71E−22 |
| ERAP1 | 0.00662153 | 1 | 1223.20296 | 0.63879531 | 2.00E−23 | 6.89E−22 |
| TTC3 | 0.00400504 | 1 | 8652.45333 | 0.34657202 | 2.03E−23 | 6.97E−22 |
| SLC44A2 | 0.00060256 | 1 | 7806.04925 | 0.35826243 | 2.10E−23 | 7.19E−22 |
| EEF1A2 | 0.00044619 | 1 | 39060.4867 | 0.32983518 | 2.27E−23 | 7.77E−22 |
| GALNT2 | 0.00054187 | 1 | 10742.2532 | 0.35962581 | 2.30E−23 | 7.86E−22 |
| PCK2 | 0.00170336 | 1 | 1275.71826 | 0.62800162 | 2.33E−23 | 7.96E−22 |
| HSPD1 | 0.00172961 | 1 | 31378.8195 | −0.3041556 | 2.37E−23 | 8.07E−22 |
| NRP2 | 3.28E−05 | 1 | 2034.00784 | −0.5014541 | 2.52E−23 | 8.56E−22 |
| PFKP | 0.00098611 | 1 | 12415.1148 | −0.3667229 | 2.95E−23 | 9.98E−22 |
| CSNK1E | 0.00073397 | 1 | 2010.6554 | −0.5129765 | 3.29E−23 | 1.11E−21 |
| H3F3B | 0.00229363 | 1 | 15427.5164 | −0.3255616 | 3.40E−23 | 1.15E−21 |
| STK38 | 0.00206126 | 1 | 1683.315 | 0.55663525 | 3.57E−23 | 1.20E−21 |
| RBM47 | 0.00049519 | 1 | 6866.87573 | 0.39652235 | 3.84E−23 | 1.29E−21 |
| LINC00511 | 0.00114007 | 1 | 872.107634 | 0.82364956 | 3.85E−23 | 1.29E−21 |
| NOP53 | 0.02957495 | 1 | 4826.52528 | 0.54539988 | 3.87E−23 | 1.29E−21 |
| TMPRSS11E | 0.00102207 | 1 | 1347.27477 | −0.6065824 | 4.16E−23 | 1.39E−21 |
| ADAM15 | 0.0037365 | 1 | 14260.3623 | 0.37937499 | 5.54E−23 | 1.84E−21 |
| MAFF | 0.00355242 | 1 | 98.022878 | −2.1778864 | 5.76E−23 | 1.91E−21 |
| FSTL3 | 7.33E−05 | 1 | 2698.22149 | 0.44529754 | 6.34E−23 | 2.10E−21 |
| IFIT3 | 0.00058958 | 1 | 260.61342 | 1.22981037 | 6.47E−23 | 2.14E−21 |
| EPS8L2 | 0.01313916 | 1 | 6831.48445 | 0.42208496 | 6.81E−23 | 2.25E−21 |
| ATP8B1 | 0.0001943 | 1 | 1486.832 | 0.57987346 | 7.33E−23 | 2.41E−21 |
| CPEB2 | 0.00072682 | 1 | 969.07752 | 0.71913841 | 7.66E−23 | 2.52E−21 |
| THSD4 | 0.00447082 | 1 | 1361.96873 | −0.5960233 | 8.73E−23 | 2.86E−21 |
| CXCL1 | 0.01944005 | 1 | 167.774511 | −1.7246271 | 9.52E−23 | 3.11E−21 |
| MAN2B1 | 0.00016466 | 1 | 835.887385 | 0.70877666 | 1.02E−22 | 3.34E−21 |
| TTC22 | 0.01162295 | 1 | 1679.60953 | 0.5824249 | 1.45E−22 | 4.73E−21 |
| CMIP | 0.00021855 | 1 | 8506.87746 | −0.3490909 | 1.56E−22 | 5.06E−21 |
| NQO1 | 0.0003533 | 1 | 46681.8086 | 0.27611112 | 1.96E−22 | 6.35E−21 |
| FLNB | 0.00052015 | 1 | 25251.9713 | −0.2923885 | 2.00E−22 | 6.48E−21 |
| JUND | 0.03307548 | 1 | 1105.32108 | 0.85232885 | 2.08E−22 | 6.73E−21 |
| ALDH3A2 | 0.00100704 | 1 | 8921.08016 | 0.3383342 | 2.34E−22 | 7.54E−21 |
| PGD | 0.00168996 | 1 | 32787.6903 | 0.3229213 | 2.49E−22 | 8.01E−21 |
| LRWD1 | 0.00023865 | 1 | 1899.04068 | −0.5065494 | 2.65E−22 | 8.53E−21 |
| FAM20C | 0.00822713 | 1 | 594.67067 | 0.86666177 | 2.73E−22 | 8.75E−21 |
| CDYL2 | 0.00215572 | 1 | 403.319466 | −0.9875224 | 3.04E−22 | 9.71E−21 |
| SMAD6 | 0.0063447 | 1 | 1179.19641 | 0.65434526 | 3.20E−22 | 1.02E−20 |
| GREB1L | 2.31E−05 | 0.75017323 | 381.583623 | −0.993891 | 3.37E−22 | 1.07E−20 |
| PLXNB2 | 0.01331138 | 1 | 11379.0998 | 0.3819066 | 3.69E−22 | 1.17E−20 |
| NRP1 | 0.00011809 | 1 | 1377.51907 | −0.5680174 | 3.71E−22 | 1.18E−20 |
| BTBD11 | 0.00103581 | 1 | 1716.39638 | 0.53078155 | 3.75E−22 | 1.19E−20 |
| MFSD6 | 0.00029836 | 1 | 921.570589 | 0.68162732 | 3.85E−22 | 1.22E−20 |
| ZNF185 | 0.00023381 | 1 | 4992.62922 | −0.3869211 | 4.38E−22 | 1.38E−20 |
| RAB15 | 5.48E−08 | 0.00178223 | 925.014777 | 0.64902628 | 4.44E−22 | 1.40E−20 |
| RREB1 | 6.76E−05 | 1 | 2684.78578 | −0.4411874 | 4.65E−22 | 1.46E−20 |
| SIK2 | 2.15E−05 | 0.69853106 | 2885.51418 | 0.42962994 | 4.67E−22 | 1.46E−20 |
| C3 | 0.0016229 | 1 | 3261.71156 | 0.4808358 | 4.81E−22 | 1.51E−20 |
| HMMR | 0.0003066 | 1 | 2502.33054 | −0.48242 | 4.91E−22 | 1.53E−20 |
| CD47 | 0.0001512 | 1 | 1842.97442 | 0.50843444 | 5.05E−22 | 1.57E−20 |
| FYCO1 | 0.00308885 | 1 | 1234.3584 | 0.60996808 | 5.57E−22 | 1.73E−20 |
| MIA2 | 0.00385709 | 1 | 2774.87916 | 0.4408457 | 6.81E−22 | 2.12E−20 |
| STK17A | 0.00137916 | 1 | 4430.77499 | −0.4180984 | 7.66E−22 | 2.37E−20 |
| GLB1 | 0.00081519 | 1 | 2026.13318 | 0.48723849 | 8.29E−22 | 2.56E−20 |
| MYLK | 0.00054557 | 1 | 2302.71611 | 1.79802944 | 5.37E−306 | 3.43E−303 |
| CCND3 | 0.0002092 | 1 | 10718.8992 | 1.12723476 | 1.00E−297 | 6.19E−295 |
| IER3 | 0.00055695 | 1 | 2813.35025 | −2.0849146 | 1.51E−296 | 9.03E−294 |
| CD24 | 0.00152357 | 1 | 1465.02973 | 2.43123154 | 3.33E−292 | 1.93E−289 |
| ST3GAL5 | 0.00074064 | 1 | 1415.99692 | −2.3775203 | 5.51E−276 | 3.09E−273 |
| ANGPTL4 | 0.00017236 | 1 | 2691.20588 | −1.7575121 | 3.15E−273 | 1.71E−270 |
| KRT80 | 7.82E−06 | 0.25145813 | 8858.73689 | 1.3141714 | 6.58E−273 | 3.48E−270 |
| ITGA2 | 3.10E−05 | 0.99407152 | 3936.92152 | −1.3954178 | 2.63E−270 | 1.35E−267 |
| TUFT1 | 5.15E−07 | 0.01658484 | 4271.09675 | 1.36122542 | 6.78E−270 | 3.39E−267 |
| FSTL3 | 3.14E−05 | 1 | 5137.33874 | 1.20686488 | 1.09E−257 | 5.29E−255 |
| CGN | 4.44E−06 | 0.14306645 | 2610.47596 | 1.53777496 | 1.80E−255 | 8.53E−253 |
| CXCL8 | 2.00E−06 | 0.06435808 | 1033.45941 | −2.5254086 | 2.59E−252 | 1.20E−249 |
| MUC1 | 0.0005985 | 1 | 4404.39877 | 1.38270945 | 1.41E−232 | 6.35E−230 |
| HMGA1 | 0.00021933 | 1 | 20839.2354 | −1.0337933 | 1.54E−232 | 6.78E−230 |
| CAVIN1 | 4.15E−06 | 0.13357116 | 53826.9292 | 0.94230179 | 2.82E−229 | 1.21E−226 |
| CAV1 | 2.15E−05 | 0.69076116 | 19898.5276 | 1.16165336 | 1.17E−225 | 4.91E−223 |
| VDR | 7.21E−06 | 0.23196518 | 2771.26318 | −1.5642731 | 3.22E−222 | 1.32E−219 |
| CA2 | 1.30E−05 | 0.41804075 | 2063.74633 | −1.6510986 | 1.01E−214 | 4.04E−212 |
| TUBA1A | 0.0008648 | 1 | 9144.52154 | 1.09801735 | 1.50E−208 | 5.89E−206 |
| KRT18 | 1.15E−05 | 0.36827008 | 7462.74202 | −1.0847312 | 8.10E−205 | 3.12E−202 |
| ACTN4 | 0.00034319 | 1 | 18978.7391 | 0.82019113 | 7.24E−203 | 2.73E−200 |
| SMOX | 0.0004666 | 1 | 2271.70771 | −1.5249102 | 3.85E−197 | 1.42E−194 |
| STAC | 1.87E−06 | 0.06022726 | 2108.18556 | 1.51115232 | 2.63E−196 | 9.54E−194 |
| DNAJB2 | 0.000264 | 1 | 4642.39814 | 1.09099987 | 4.49E−195 | 1.60E−192 |
| MALSU1 | 7.83E−06 | 0.25194317 | 2829.24003 | 1.36525728 | 2.44E−194 | 8.51E−192 |
| MRAS | 5.75E−06 | 0.18490751 | 3323.2565 | 1.24406882 | 1.74E−189 | 5.95E−187 |
| COL12A1 | 0.00021528 | 1 | 1376.22704 | 1.70428185 | 2.60E−187 | 8.74E−185 |
| DOCK4 | 0.00041544 | 1 | 3763.63749 | −1.2122926 | 1.46E−184 | 4.83E−182 |
| BCAM | 0.00174953 | 1 | 4151.52344 | 1.3674586 | 3.01E−182 | 9.77E−180 |
| CLU | 1.59E−05 | 0.51045644 | 18019.8989 | 0.90190688 | 1.15E−176 | 3.67E−174 |
| TUBA4A | 1.13E−05 | 0.36313503 | 15569.6504 | 0.93309104 | 1.31E−174 | 4.10E−172 |
| PRDX6 | 2.44E−06 | 0.0786108 | 13589.1745 | 0.81723485 | 4.60E−172 | 1.42E−169 |
| LXN | 0.00057349 | 1 | 1610.90771 | −1.6846858 | 5.49E−171 | 1.67E−168 |
| FBLIM1 | 0.00032569 | 1 | 3069.69005 | 1.21393904 | 3.94E−169 | 1.18E−166 |
| NCEH1 | 0.00088192 | 1 | 7715.23668 | −1.0854652 | 6.48E−169 | 1.90E−166 |
| KIAA1522 | 3.88E−06 | 0.12483506 | 8430.66021 | 0.82129327 | 3.39E−168 | 9.80E−166 |
| OPTN | 0.00016654 | 1 | 7413.09262 | 0.92352135 | 1.85E−163 | 5.26E−161 |
| OLFM2 | 6.19E−05 | 1 | 1847.99778 | 1.54434432 | 2.95E−160 | 8.26E−158 |
| SRGN | 0.00019616 | 1 | 13490.7391 | −0.7715467 | 1.36E−159 | 3.77E−157 |
| CRIP2 | 3.51E−06 | 0.11293613 | 1354.89017 | 1.67143258 | 1.68E−159 | 4.56E−157 |
| SUSD2 | 0.00515166 | 1 | 1629.08184 | −1.7880424 | 2.20E−157 | 5.89E−155 |
| CTIF | 0.00055564 | 1 | 3773.15994 | 1.09255031 | 8.86E−156 | 2.34E−153 |
| CDH4 | 0.00016109 | 1 | 1367.49672 | −1.544156 | 1.24E−152 | 3.22E−150 |
| OXTR | 8.43E−06 | 0.27125319 | 831.015608 | 2.14546046 | 1.65E−148 | 4.24E−146 |
| CITED2 | 0.00173397 | 1 | 6796.8234 | 1.01165605 | 5.26E−148 | 1.33E−145 |
| HLA-B | 0.00014881 | 1 | 12156.2061 | 0.88789263 | 5.65E−147 | 1.41E−144 |
| DSTN | 0.00132484 | 1 | 8720.30925 | 0.86713134 | 5.26E−144 | 1.30E−141 |
| PHLDB2 | 0.00012503 | 1 | 5272.45055 | 0.89263648 | 2.50E−143 | 6.10E−141 |
| NDST1 | 0.00228005 | 1 | 5711.16778 | 0.95094463 | 6.34E−143 | 1.52E−140 |
| SPX | 4.26E−06 | 0.13701399 | 1736.47271 | −1.3359379 | 3.83E−142 | 9.08E−140 |
| DAG1 | 2.66E−05 | 0.85439344 | 17975.1393 | 0.76892319 | 4.06E−141 | 9.50E−139 |
| PDLIM1 | 2.26E−05 | 0.72524203 | 9132.81201 | 0.77278281 | 1.44E−140 | 3.33E−138 |
| TMSB4X | 0.0030151 | 1 | 20586.3772 | 0.85444474 | 1.59E−139 | 3.64E−137 |
| ZFAND5 | 6.87E−06 | 0.22096643 | 6642.82457 | 0.87534074 | 9.37E−138 | 2.11E−135 |
| SEMA4B | 3.98E−05 | 1 | 6460.42922 | −1.0674915 | 7.30E−136 | 1.63E−133 |
| ABHD4 | 2.77E−05 | 0.88959352 | 8683.12198 | 0.80813486 | 1.84E−135 | 4.05E−133 |
| MYH9 | 0.00043314 | 1 | 56425.2153 | 0.72251061 | 6.31E−135 | 1.37E−132 |
| THBS3 | 0.0003214 | 1 | 3299.97262 | 1.21022367 | 5.07E−134 | 1.09E−131 |
| PSMB9 | 3.06E−05 | 0.98223556 | 3468.55079 | 1.04872483 | 3.14E−132 | 6.68E−130 |
| PSAP | 0.00106975 | 1 | 48504.9572 | 0.81063024 | 7.12E−132 | 1.50E−129 |
| TRIM2 | 5.59E−05 | 1 | 1171.12222 | −1.5098313 | 7.82E−130 | 1.63E−127 |
| MAP1LC3A | 0.00033244 | 1 | 1075.16015 | 1.56845742 | 1.69E−129 | 3.47E−127 |
| ARID5B | 5.86E−05 | 1 | 2849.80733 | 1.2884479 | 8.67E−129 | 1.76E−126 |
| UPP1 | 7.08E−07 | 0.02280558 | 2179.7187 | −1.1376656 | 4.85E−128 | 9.74E−126 |
| PIEZO1 | 1.34E−05 | 0.43152095 | 7928.74264 | 0.78701524 | 1.09E−127 | 2.16E−125 |
| SAT1 | 9.25E−05 | 1 | 2138.41221 | −1.2734729 | 1.87E−127 | 3.68E−125 |
| MMP24 | 7.24E−05 | 1 | 329.651417 | 3.22433972 | 2.29E−127 | 4.45E−125 |
| CNTNAP1 | 0.00082312 | 1 | 2114.20053 | 1.21495462 | 3.19E−127 | 6.16E−125 |
| CADM1 | 4.73E−06 | 0.15225666 | 1845.40282 | 1.21713478 | 3.08E−126 | 5.88E−124 |
| CCN1 | 0.00037297 | 1 | 35756.6127 | 0.75857925 | 4.19E−126 | 7.91E−124 |
| RHOB | 0.00021128 | 1 | 5309.47192 | 1.15235714 | 3.88E−125 | 7.24E−123 |
| UPK3B | 0.00048888 | 1 | 307.520039 | 3.40972449 | 9.89E−125 | 1.83E−122 |
| SEMA3C | 0.00025649 | 1 | 13724.7801 | 0.69405291 | 1.70E−124 | 3.11E−122 |
| TNS3 | 0.00019229 | 1 | 13155.564 | 0.81093882 | 7.64E−124 | 1.39E−121 |
| TM4SF1 | 0.00228732 | 1 | 17185.7195 | −0.7146014 | 7.39E−123 | 1.33E−120 |
| COL4A4 | 0.00426049 | 1 | 5435.76879 | 1.08091014 | 2.66E−122 | 4.74E−120 |
| JAK1 | 0.00082597 | 1 | 12851.2287 | 0.69778828 | 3.75E−122 | 6.60E−120 |
| SLC4A7 | 0.00012297 | 1 | 1991.69901 | −1.2218681 | 8.91E−122 | 1.56E−119 |
| CRISPLD1 | 0.00012118 | 1 | 5258.02392 | 0.81591989 | 1.60E−120 | 2.77E−118 |
| CD151 | 0.00161831 | 1 | 6214.96112 | 0.81456521 | 1.90E−120 | 3.26E−118 |
| AKR1B1 | 0.00072143 | 1 | 21130.9502 | 0.86123551 | 4.12E−120 | 7.00E−118 |
| ERGIC1 | 0.00117551 | 1 | 9328.74857 | 0.8099079 | 6.86E−120 | 1.15E−117 |
| MAP1B | 0.00036211 | 1 | 10133.8398 | 0.73307779 | 5.37E−119 | 8.95E−117 |
| EPGN | 0.00029592 | 1 | 14024.857 | −0.7550815 | 1.29E−118 | 2.12E−116 |
| CDCP1 | 2.78E−05 | 0.89215169 | 8933.91303 | −0.8371534 | 1.39E−117 | 2.27E−115 |
| PTPN12 | 0.00029469 | 1 | 6086.45177 | −0.7819888 | 2.52E−117 | 4.09E−115 |
| TGFB2 | 4.64E−05 | 1 | 4037.55803 | 1.10344578 | 1.30E−116 | 2.09E−114 |
| PLK2 | 8.20E−06 | 0.2637925 | 2386.20469 | 1.06475206 | 1.45E−116 | 2.32E−114 |
| PIEZO2 | 0.00012736 | 1 | 6353.9577 | 0.8249331 | 2.52E−116 | 3.99E−114 |
| GPR37 | 2.64E−05 | 0.84848259 | 2623.80155 | −1.090176 | 1.39E−115 | 2.18E−113 |
| COL5A1 | 0.00440476 | 1 | 1425.91836 | 1.57311187 | 3.25E−115 | 5.05E−113 |
| PTGES | 3.76E−05 | 1 | 1659.07289 | −1.2094279 | 3.57E−115 | 5.50E−113 |
| GNG11 | 0.00012912 | 1 | 3038.57435 | −1.0792464 | 5.13E−115 | 7.85E−113 |
| SPRED1 | 2.69E−06 | 0.08660294 | 1407.31903 | −1.2937039 | 1.27E−114 | 1.92E−112 |
| PRAME | 0.0003528 | 1 | 7323.06376 | −0.7723091 | 2.83E−114 | 4.25E−112 |
| RSU1 | 3.38E−05 | 1 | 4024.17383 | 0.89041748 | 3.50E−114 | 5.22E−112 |
| MAP3K21 | 5.08E−06 | 0.16351568 | 1160.43199 | 1.44688602 | 5.73E−114 | 8.48E−112 |
| LSS | 0.00062011 | 1 | 4459.99203 | 0.83003406 | 4.55E−113 | 6.68E−111 |
| ZNF185 | 0.00017178 | 1 | 2203.60468 | 1.0553769 | 3.58E−112 | 5.22E−110 |
| SYNPO | 0.00144179 | 1 | 8567.78073 | 0.72213419 | 4.28E−112 | 6.18E−110 |
| IFITM2 | 4.59E−05 | 1 | 8473.42473 | −0.7262248 | 3.26E−111 | 4.67E−109 |
| DAPK1 | 9.73E−05 | 1 | 2370.1112 | 1.01583342 | 1.16E−110 | 1.65E−108 |
| RGS2 | 0.0041589 | 1 | 996.573173 | −1.7144993 | 5.51E−109 | 7.77E−107 |
| SPRY2 | 0.00028479 | 1 | 625.358348 | −1.9299534 | 9.15E−109 | 1.28E−106 |
| FOSL2 | 0.00037776 | 1 | 9446.23834 | 0.75842149 | 1.14E−108 | 1.58E−106 |
| MYL6 | 3.67E−05 | 1 | 16551.0605 | 0.6082463 | 4.06E−108 | 5.61E−106 |
| ANKRD1 | 0.00238364 | 1 | 1764.60721 | 1.23542969 | 7.71E−108 | 1.06E−105 |
| CAPN2 | 5.47E−06 | 0.17611382 | 14537.6125 | 0.61860022 | 1.01E−107 | 1.37E−105 |
| NQO1 | 1.65E−05 | 0.53144407 | 82172.3456 | −0.5927663 | 2.45E−106 | 3.30E−104 |
| DMD | 6.48E−05 | 1 | 1778.37073 | −1.142127 | 1.28E−105 | 1.72E−103 |
| EEF1A2 | 0.00266244 | 1 | 28236.9099 | 0.68120886 | 2.16E−105 | 2.87E−103 |
| FOS | 0.00145015 | 1 | 392.400419 | −2.6664795 | 6.20E−105 | 8.19E−103 |
| ABCB1 | 0.00013099 | 1 | 569.679146 | 1.92990064 | 1.26E−103 | 1.65E−101 |
| TIMP1 | 0.00023681 | 1 | 2214.89487 | −1.0334411 | 3.43E−103 | 4.47E−101 |
| PLD3 | 3.28E−05 | 1 | 12488.5197 | 0.73738609 | 1.35E−102 | 1.74E−100 |
| TTC7A | 2.70E−05 | 0.86600486 | 4122.41369 | 0.85197984 | 1.60E−102 | 2.05E−100 |
| FMNL2 | 3.97E−06 | 0.12792593 | 5076.37548 | −0.7541239 | 2.35E−101 | 2.99E−99 |
| PLEKHA6 | 2.60E−06 | 0.08369373 | 1636.6821 | −1.1473997 | 3.05E−101 | 3.86E−99 |
| LAMB2 | 8.36E−05 | 1 | 10365.1595 | 0.86855369 | 4.91E−100 | 6.18E−98 |
| KIF1C | 0.00064171 | 1 | 7847.43011 | 0.73287409 | 1.94E−99 | 2.42E−97 |
| JUND | 0.00043741 | 1 | 4367.61423 | 0.96642478 | 3.18E−99 | 3.94E−97 |
| BASP1 | 0.0012465 | 1 | 29448.5585 | 0.57865266 | 3.50E−99 | 4.31E−97 |
| ABCA3 | 3.26E−05 | 1 | 2755.32438 | 1.03662172 | 4.43E−99 | 5.42E−97 |
| SPRY4 | 0.0024278 | 1 | 923.226196 | −1.5922643 | 6.88E−99 | 8.37E−97 |
| PEA15 | 0.00028625 | 1 | 8013.05782 | 0.75872397 | 3.32E−98 | 4.01E−96 |
| SNHG3 | 0.00059884 | 1 | 2713.26142 | −0.9301745 | 4.66E−98 | 5.59E−96 |
| CYTH3 | 0.00017473 | 1 | 2646.2088 | 0.91628676 | 8.25E−98 | 9.84E−96 |
| DUSP6 | 0.00154912 | 1 | 387.697946 | −2.4668691 | 1.34E−97 | 1.59E−95 |
| SYNGR3 | 0.00312346 | 1 | 448.456783 | 2.20295381 | 7.99E−97 | 9.42E−95 |
| PDE8A | 0.0002156 | 1 | 5139.98284 | −0.8245455 | 4.71E−96 | 5.52E−94 |
| ICAM1 | 3.90E−06 | 0.12555857 | 1668.12725 | −1.081417 | 1.56E−95 | 1.81E−93 |
| DUSP4 | 0.00128461 | 1 | 3922.72476 | −1.0493375 | 4.99E−95 | 5.77E−93 |
| FSIP2 | 0.00018503 | 1 | 729.469322 | −1.8566546 | 5.45E−95 | 6.26E−93 |
| PPP1R13L | 0.0004873 | 1 | 2422.21171 | 0.97115375 | 1.80E−93 | 2.05E−91 |
| PLCD3 | 0.00100141 | 1 | 2588.77227 | 0.93051153 | 3.10E−93 | 3.52E−91 |
| CD44 | 0.0012174 | 1 | 7969.27406 | −0.6810569 | 3.21E−92 | 3.62E−90 |
| BMP6 | 2.59E−05 | 0.83006241 | 2166.01128 | −0.9732498 | 6.73E−92 | 7.54E−90 |
| NPR3 | 0.00118545 | 1 | 4024.53147 | 0.80838706 | 3.81E−91 | 4.25E−89 |
| LATS2 | 0.00053985 | 1 | 2611.536 | 0.89424981 | 7.87E−91 | 8.71E−89 |
| SPRED2 | 4.92E−05 | 1 | 3567.81252 | −0.853602 | 9.59E−90 | 1.06E−87 |
| HACD2 | 0.00027524 | 1 | 5978.94981 | 0.77284523 | 1.25E−89 | 1.37E−87 |
| ERBB2 | 0.00106536 | 1 | 6025.1491 | 0.6769644 | 1.92E−89 | 2.09E−87 |
| DNMBP | 0.00184207 | 1 | 3286.83577 | −0.9651964 | 3.55E−89 | 3.84E−87 |
| CAV2 | 0.00038874 | 1 | 3928.46044 | 0.83184629 | 6.14E−89 | 6.60E−87 |
| COL8A1 | 1.45E−06 | 0.04670396 | 7947.69724 | 0.61407717 | 1.98E−88 | 2.12E−86 |
| MYEOV | 0.00055755 | 1 | 929.073097 | −1.6306074 | 2.05E−88 | 2.18E−86 |
| DUSP3 | 3.60E−05 | 1 | 4306.79562 | 0.7282253 | 4.22E−88 | 4.46E−86 |
| TNFAIP1 | 3.73E−05 | 1 | 6363.80677 | 0.68034546 | 4.29E−88 | 4.51E−86 |
| RIN1 | 1.76E−05 | 0.56597984 | 1005.25013 | −1.3506695 | 8.31E−88 | 8.69E−86 |
| DLC1 | 4.04E−07 | 0.01300393 | 1523.23696 | 1.04546767 | 4.05E−87 | 4.21E−85 |
| AJUBA | 5.20E−05 | 1 | 7454.22179 | 0.75136679 | 5.70E−87 | 5.89E−85 |
| CREM | 0.00019408 | 1 | 1807.11493 | −0.994283 | 1.02E−86 | 1.05E−84 |
| SCARA3 | 0.00017172 | 1 | 920.950959 | 1.31903879 | 1.52E−86 | 1.56E−84 |
| TRIM16L | 1.58E−05 | 0.50883329 | 5443.84205 | 0.66552139 | 1.54E−86 | 1.57E−84 |
| DHCR24 | 3.75E−05 | 1 | 19127.9525 | 0.60654439 | 3.65E−86 | 3.68E−84 |
| PHLDB1 | 0.00204379 | 1 | 2697.6808 | 0.87556809 | 8.54E−86 | 8.58E−84 |
| MFSD12 | 7.17E−06 | 0.23066385 | 2585.92561 | 0.85624713 | 1.20E−85 | 1.20E−83 |
| LUCAT1 | 0.00262076 | 1 | 1504.63114 | −1.2216905 | 3.33E−85 | 3.31E−83 |
| CALD1 | 0.00163434 | 1 | 7679.55124 | 0.81867042 | 9.47E−85 | 9.36E−83 |
| PLAUR | 0.00034702 | 1 | 1834.03572 | −1.0928199 | 1.97E−84 | 1.93E−82 |
| TRIB3 | 0.00012046 | 1 | 2044.97662 | −1.0310067 | 9.69E−84 | 9.48E−82 |
| TUBB2A | 8.85E−05 | 1 | 1334.72319 | 1.12930792 | 7.58E−83 | 7.38E−81 |
| AFAP1 | 2.26E−05 | 0.72417173 | 2259.0021 | 0.91676473 | 7.17E−82 | 6.94E−80 |
| ARHGAP12 | .00019744 | 1 | 3233.43873 | −0.8028413 | 9.82E−82 | 9.45E−80 |
| TUBB6 | 0.00428198 | 1 | 4528.16072 | 0.85457257 | 1.02E−81 | 9.79E−80 |
| STIM1 | 2.18E−06 | 0.07023277 | 4242.75298 | 0.69193664 | 1.24E−81 | 1.19E−79 |
| HLA-C | 0.00080537 | 1 | 10426.046 | 0.70502073 | 1.76E−81 | 1.67E−79 |
| XDH | 0.00010709 | 1 | 872.186571 | −1.3328667 | 4.13E−81 | 3.89E−79 |
| F2RL2 | 0.00026255 | 1 | 5554.70722 | 0.66451051 | 4.26E−81 | 4.00E−79 |
| TIMP2 | 0.00053458 | 1 | 7297.60048 | 0.629407 | 1.03E−80 | 9.60E−79 |
| TNKS1BP1 | 0.00216564 | 1 | 8325.99417 | 0.64647677 | 1.13E−80 | 1.05E−78 |
| CAMK1D | 0.00500959 | 1 | 2561.49254 | 1.03486131 | 2.08E−80 | 1.93E−78 |
| NR4A3 | 1.51E−05 | 0.48419503 | 996.32682 | 1.27827693 | 5.95E−80 | 5.48E−78 |
| LTBP4 | 0.00245538 | 1 | 5409.63308 | 0.92132167 | 8.09E−80 | 7.41E−78 |
| FBXO27 | 7.18E−05 | 1 | 6464.56313 | −0.7527178 | 9.64E−80 | 8.78E−78 |
| SOCS3 | 0.00171032 | 1 | 1415.91789 | −1.2980366 | 2.70E−79 | 2.45E−77 |
| LRATD2 | 2.61E−06 | 0.08414573 | 3872.44961 | −0.7139681 | 5.13E−79 | 4.62E−77 |
| TSC22D3 | 0.00011041 | 1 | 1751.88915 | 1.06043571 | 6.88E−79 | 6.18E−77 |
| INF2 | 0.00155328 | 1 | 4104.44726 | 0.72348218 | 9.79E−79 | 8.75E−77 |
| JUP | 4.45E−05 | 1 | 33291.2749 | −0.4923969 | 1.61E−78 | 1.43E−76 |
| TBC1D8 | 0.00074166 | 1 | 2971.7567 | −0.9851472 | 3.76E−78 | 3.32E−76 |
| CERCAM | 0.00127426 | 1 | 2099.11877 | 1.00920584 | 5.67E−78 | 4.99E−76 |
| MLX | 7.31E−05 | 1 | 3314.66009 | −0.760747 | 6.35E−78 | 5.57E−76 |
| NT5C2 | 0.00217191 | 1 | 2758.35008 | 0.86901827 | 1.06E−77 | 9.23E−76 |
| TRAM1 | 0.00100295 | 1 | 7518.82719 | 0.86016381 | 1.21E−77 | 1.05E−75 |
| ANXA2 | 0.00052493 | 1 | 48358.235 | 0.51093996 | 1.46E−77 | 1.26E−75 |
| YWHAH | 7.93E−06 | 0.2550674 | 5685.63546 | 0.63314818 | 1.60E−77 | 1.37E−75 |
| FOXL1 | 0.00230548 | 1 | 919.505256 | −1.3240129 | 1.68E−77 | 1.44E−75 |
| HLA-A | 0.00174801 | 1 | 11062.1695 | 0.60090339 | 2.24E−77 | 1.91E−75 |
| GPSM3 | 1.30E−05 | 0.41769816 | 1323.80098 | −1.0861268 | 2.98E−77 | 2.53E−75 |
| EFNA1 | 0.00214806 | 1 | 5924.78542 | 0.72513923 | 3.74E−77 | 3.16E−75 |
| SLC4A2 | 0.00017205 | 1 | 8381.1822 | 0.65612294 | 3.91E−77 | 3.28E−75 |
| PMEPA1 | 0.00132643 | 1 | 11456.0666 | 0.60680548 | 5.10E−77 | 4.27E−75 |
| CPA4 | 4.41E−06 | 0.14191035 | 6297.56016 | 0.61266373 | 8.12E−77 | 6.76E−75 |
| PRKAB2 | 0.00013868 | 1 | 2967.01359 | 0.75612336 | 1.71E−76 | 1.42E−74 |
| NBR1 | 0.00075164 | 1 | 5675.15767 | −0.7030302 | 2.60E−76 | 2.14E−74 |
| VEGFC | 4.36E−05 | 1 | 1556.82711 | −1.0421263 | 3.49E−76 | 2.87E−74 |
| RASD1 | 0.00103764 | 1 | 513.703734 | −1.8625088 | 4.37E−76 | 3.58E−74 |
| COL4A3 | 5.84E−05 | 1 | 2329.59085 | 0.93836883 | 4.55E−76 | 3.70E−74 |
| TLE6 | 5.34E−05 | 1 | 418.097802 | −1.9650278 | 7.25E−76 | 5.88E−74 |
| SAMD4A | 3.00E−05 | 0.96213198 | 4401.53158 | 0.72542339 | 3.06E−75 | 2.47E−73 |
| MRTFA | 0.0021662 | 1 | 3554.32643 | 0.77811499 | 3.23E−75 | 2.59E−73 |
| VAT1 | 0.00262238 | 1 | 11748.587 | 0.64420236 | 3.23E−75 | 2.59E−73 |
| FOSL1 | 2.58E−05 | 0.82955076 | 1242.56173 | −1.1382077 | 3.61E−75 | 2.87E−73 |
| ORMDL3 | 2.69E−05 | 0.86445043 | 2977.24887 | 0.80314773 | 4.22E−75 | 3.35E−73 |
| ANKRD28 | 0.0013284 | 1 | 3706.66548 | −0.7223907 | 1.00E−74 | 7.94E−73 |
| TMEM131 | 1.56E−05 | 0.50120792 | 7914.11895 | −0.5869088 | 1.32E−74 | 1.04E−72 |
| APLP1 | 2.88E−05 | 0.92334259 | 5478.1918 | 0.67933407 | 1.68E−74 | 1.31E−72 |
| SCEL | 6.66E−05 | 1 | 1613.79296 | −1.0887772 | 1.97E−74 | 1.54E−72 |
| RIPK4 | 0.00017624 | 1 | 1484.46311 | −0.9861622 | 2.02E−74 | 1.57E−72 |
| RHOBTB1 | 0.00186894 | 1 | 1291.69109 | 1.11131575 | 3.14E−74 | 2.43E−72 |
| LAT2 | 5.24E−05 | 1 | 2118.1093 | −0.8928933 | 4.39E−74 | 3.38E−72 |
| SERINC3 | 1.92E−05 | 0.61549189 | 8942.70492 | −0.5700828 | 7.08E−74 | 5.43E−72 |
| ID3 | 0.00100644 | 1 | 8512.45789 | 1.35824494 | 1.68E−73 | 1.28E−71 |
| AGRN | 0.00549002 | 1 | 21610.2599 | 0.69924832 | 2.22E−73 | 1.69E−71 |
| FRMD4A | 0.00266052 | 1 | 338.72186 | −2.2010911 | 2.42E−73 | 1.83E−71 |
| CCDC85C | 0.00016566 | 1 | 2175.45444 | 0.86400415 | 4.37E−73 | 3.30E−71 |
| SDC4 | 0.00026619 | 1 | 11951.0177 | 0.56778286 | 6.90E−73 | 5.19E−71 |
| SLC7A5 | 0.00054085 | 1 | 15811.5666 | 0.57752547 | 9.04E−73 | 6.77E−71 |
| EML2 | 0.00037333 | 1 | 1427.16279 | −1.0154627 | 1.58E−72 | 1.18E−70 |
| SMPD1 | 0.00193057 | 1 | 3057.19057 | 0.8060467 | 1.98E−72 | 1.47E−70 |
| TAPBP | 0.00033846 | 1 | 14053.2703 | 0.68435721 | 2.24E−72 | 1.66E−70 |
| RUSC2 | 0.00035214 | 1 | 3051.68104 | 0.77613228 | 2.66E−72 | 1.96E−70 |
| RAI14 | 7.57E−05 | 1 | 7209.87123 | 0.6117779 | 4.24E−72 | 3.11E−70 |
| CAB39 | 1.41E−06 | 0.04531659 | 2780.97329 | −0.758433 | 5.59E−72 | 4.09E−70 |
| ARHGEF17 | 1.17E−06 | 0.03762322 | 2067.49116 | 0.83039318 | 2.12E−71 | 1.54E−69 |
| NOTCH2 | 0.00512287 | 1 | 2533.04489 | 0.93569541 | 2.73E−71 | 1.98E−69 |
| GRN | 0.00199705 | 1 | 7374.77006 | 0.63336341 | 2.86E−71 | 2.06E−69 |
| TPM2 | 1.51E−05 | 0.48388443 | 1065.6901 | 1.1486995 | 2.97E−71 | 2.14E−69 |
| GAA | 0.00029904 | 1 | 3643.25376 | 0.88394256 | 7.38E−71 | 5.29E−69 |
| IRF2BPL | 0.00164602 | 1 | 6517.72545 | −0.8097503 | 1.86E−70 | 1.33E−68 |
| FADS2 | 9.34E−05 | 1 | 17981.7805 | −0.5533534 | 2.48E−70 | 1.77E−68 |
| PITPNC1 | 0.0021755 | 1 | 3828.41614 | −0.7671437 | 2.99E−70 | 2.12E−68 |
| GBP1 | 0.00013863 | 1 | 1263.03156 | 1.01356349 | 3.28E−70 | 2.32E−68 |
| SELENOM | 0.00013717 | 1 | 3354.56619 | 0.69693112 | 2.22E−69 | 1.56E−67 |
| ADGRB2 | 1.41E−05 | 0.45191364 | 2671.1276 | 0.7827009 | 2.74E−69 | 1.92E−67 |
| CKB | 0.00255784 | 1 | 1614.7797 | 1.07350958 | 3.35E−69 | 2.34E−67 |
| AMOTL2 | 0.00377502 | 1 | 8269.42521 | 0.67863664 | 3.99E−69 | 2.77E−67 |
| KIAA0319 | 0.00368563 | 1 | 5865.06045 | 0.64642644 | 7.03E−69 | 4.87E−67 |
| TRAM2 | 0.00235888 | 1 | 3422.51743 | 0.74293789 | 7.05E−69 | 4.87E−67 |
| ERO1A | 0.00032465 | 1 | 8875.9775 | −0.6515485 | 7.87E−69 | 5.41E−67 |
| THSD4 | 0.00014738 | 1 | 8539.21835 | −0.702746 | 9.52E−69 | 6.52E−67 |
| HEATR5A | 0.0022225 | 1 | 2123.65128 | 0.86602197 | 2.32E−68 | 1.59E−66 |
| MYORG | 0.00031336 | 1 | 2256.99376 | 0.79145215 | 3.38E−68 | 2.30E−66 |
| 4-Mar | 0.00165446 | 1 | 2472.41045 | −0.8199784 | 7.07E−68 | 4.79E−66 |
| STK38 | 0.00205286 | 1 | 5047.34065 | 0.65995649 | 7.40E−68 | 5.00E−66 |
| MAP4 | 2.77E−05 | 0.88746586 | 17795.9959 | 0.48256116 | 7.43E−68 | 5.00E−66 |
| MAP2 | 3.94E−05 | 1 | 15018.1403 | 0.49666881 | 7.58E−68 | 5.08E−66 |
| PLLP | 3.78E−05 | 1 | 2975.07331 | −0.7666617 | 7.71E−68 | 5.15E−66 |
| GDF15 | 0.00217589 | 1 | 212.503718 | −2.9204751 | 1.43E−67 | 9.51E−66 |
| ZYX | 0.00452793 | 1 | 7704.31561 | 0.62038769 | 2.30E−67 | 1.52E−65 |
| NPAS2 | 0.00035197 | 1 | 1003.07282 | −1.1358301 | 1.03E−66 | 6.81E−65 |
| AKR1C2 | 0.00297255 | 1 | 9213.93764 | 0.76084881 | 1.86E−66 | 1.23E−64 |
| B2M | 4.73E−05 | 1 | 12482.2958 | 0.52166996 | 9.82E−66 | 6.44E−64 |
| HPS5 | 0.00094483 | 1 | 5260.56864 | 0.69907345 | 1.39E−65 | 9.11E−64 |
| MYOCD | 0.00076664 | 1 | 412.015547 | 1.71037223 | 1.45E−65 | 9.45E−64 |
| KLF7 | 0.00476798 | 1 | 1675.0689 | 0.96508933 | 1.47E−65 | 9.54E−64 |
| DCBLD2 | 0.00084584 | 1 | 6429.77123 | −0.6799964 | 2.00E−65 | 1.29E−63 |
| TMEM156 | 2.97E−05 | 0.95321525 | 548.033063 | −1.5917878 | 2.08E−65 | 1.34E−63 |
| RHPN2 | 9.76E−05 | 1 | 2790.26223 | −0.7101168 | 2.10E−65 | 1.35E−63 |
| MTCH1 | 0.0005479 | 1 | 8789.44457 | 0.59065683 | 4.03E−65 | 2.58E−63 |
| TMEM132A | 0.00010373 | 1 | 3532.21916 | 0.71646045 | 4.31E−65 | 2.75E−63 |
| IL4R | 9.63E−05 | 1 | 1278.31959 | −0.984996 | 5.82E−65 | 3.70E−63 |
| COL18A1 | 0.0044828 | 1 | 1265.61404 | 1.18238028 | 8.64E−65 | 5.47E−63 |
| RELB | 0.00601827 | 1 | 1838.29721 | 0.99960778 | 9.73E−65 | 6.14E−63 |
| SIPA1L2 | 0.00032852 | 1 | 3268.27617 | −0.767098 | 9.88E−65 | 6.22E−63 |
| IL6R | 0.00048922 | 1 | 1764.89444 | −0.8912114 | 1.07E−64 | 6.70E−63 |
| TRIM37 | 0.00201064 | 1 | 4004.51716 | 0.76579444 | 1.11E−64 | 6.91E−63 |
| CABLES1 | 5.90E−05 | 1 | 1451.03425 | 0.94795798 | 2.65E−64 | 1.65E−62 |
| FN1 | 0.00020328 | 1 | 2885.53145 | 0.93548295 | 2.86E−64 | 1.77E−62 |
| CDV3 | 9.65E−05 | 1 | 10257.664 | 0.56042664 | 3.08E−64 | 1.90E−62 |
| UCHL1 | 2.00E−05 | 0.64081999 | 7380.19486 | 0.52579585 | 3.36E−64 | 2.07E−62 |
| MBP | 0.00050046 | 1 | 1677.12084 | −0.8714662 | 3.44E−64 | 2.11E−62 |
| PARVA | 0.00023948 | 1 | 1356.05379 | 0.93691778 | 5.67E−64 | 3.47E−62 |
| PGK1 | 0.00022386 | 1 | 19791.1445 | −0.4545289 | 6.92E−64 | 4.22E−62 |
| PALM | 0.00025608 | 1 | 1770.81185 | 0.90354241 | 8.60E−64 | 5.23E−62 |
| HSP90AA1 | 0.00359492 | 1 | 124413.856 | −0.5838047 | 1.75E−63 | 1.06E−61 |
| RGP1 | 0.00029418 | 1 | 2173.12908 | −0.7962956 | 2.18E−63 | 1.32E−61 |
| ECE1 | 0.00166592 | 1 | 9905.19881 | 0.52440987 | 2.54E−53 | 1.53E−61 |
| DYNC1H1 | 0.00162403 | 1 | 65654.4116 | −0.5001753 | 6.90E−63 | 4.14E−61 |
| GPR3 | 0.00022842 | 1 | 305.582685 | −2.0854997 | 8.76E−63 | 5.24E−61 |
| CTSA | 0.00019821 | 1 | 5787.33199 | 0.55747233 | 9.80E−63 | 5.84E−61 |
| ECT2 | 0.00030536 | 1 | 8160.11368 | −0.6852185 | 1.62E−62 | 9.64E−61 |
| TP53I3 | 4.83E−05 | 1 | 2674.85944 | 0.77949341 | 2.16E−62 | 1.28E−60 |
| NISCH | 0.00059784 | 1 | 3433.56766 | 0.71801305 | 3.27E−62 | 1.93E−60 |
| C3 | 0.00249246 | 1 | 892.182236 | 1.1576812 | 3.60E−62 | 2.12E−60 |
| TES | 0.00016609 | 1 | 5338.30708 | 0.69147325 | 9.18E−62 | 5.39E−60 |
| TOR4A | 0.00087764 | 1 | 2059.26502 | −0.792776 | 1.12E−61 | 6.57E−60 |
| CLSTN3 | 0.00299376 | 1 | 4027.8352 | 0.72385493 | 1.34E−61 | 7.84E−60 |
| SLC2A8 | 0.00015715 | 1 | 1013.13518 | 1.07036191 | 1.40E−61 | 8.16E−60 |
| MTHFD2L | 0.00185392 | 1 | 660.817178 | −1.3380879 | 2.50E−61 | 1.44E−59 |
| VSIR | 0.00013577 | 1 | 1041.76894 | 1.18500102 | 2.49E−61 | 1.44E−59 |
| AC008875.3 | 3.55E−05 | 1 | 763.21556 | −1.2963007 | 2.89E−51 | 1.67E−59 |
| SQLE | 0.002575 | 1 | 5857.61198 | 0.61944265 | 3.39E−61 | 1.94E−59 |
| HIF1A | 0.00020004 | 1 | 9549.08678 | −0.5205722 | 4.01E−61 | 2.29E−59 |
| PLXNB2 | 0.00710008 | 1 | 6640.5552 | 0.63729022 | 4.07E−61 | 2.32E−59 |
| FOXC2 | 5.66E−05 | 1 | 1106.61484 | −1.1080245 | 4.58E−61 | 2.61E−59 |
| CCDC68 | 0.00014802 | 1 | 866.404829 | −1.137006 | 1.15E−60 | 6.55E−59 |
| AP1M2 | 4.17E−05 | 1 | 2481.03811 | 0.71182426 | 2.49E−60 | 1.41E−58 |
| C1orf198 | 0.00054531 | 1 | 2463.30008 | 0.73273467 | 3.25E−60 | 1.83E−58 |
| ACOX2 | 0.00677838 | 1 | 165.29519 | −4.6614881 | 4.28E−60 | 2.41E−58 |
| LAMP3 | 0.0002172 | 1 | 728.183471 | −1.2434623 | 4.43E−60 | 2.48E−58 |
| NHSL1 | 4.48E−06 | 0.14410848 | 1222.8449 | −0.9600022 | 4.52E−60 | 2.53E−58 |
| ELK3 | 0.00057638 | 1 | 2687.08273 | −0.7399508 | 6.28E−60 | 3.50E−58 |
| PCDH9 | 6.72E−05 | 1 | 907.375939 | 1.20405554 | 9.14E−60 | 5.08E−58 |
| LRP8 | 0.00012814 | 1 | 1848.80389 | −0.8259642 | 5.65E−59 | 3.13E−57 |
| HMGB3 | 0.00021785 | 1 | 8712.77996 | −0.5256606 | 8.28E−58 | 4.57E−56 |
| COL11A2 | 0.01042511 | 1 | 384.307907 | 1.88143826 | 9.19E−58 | 5.06E−56 |
| CNN1 | 0.00059585 | 1 | 129.041239 | 4.47148411 | 1.11E−57 | 6.07E−56 |
| OAS1 | 5.39E−05 | 1 | 2577.16362 | −0.752715 | 1.24E−57 | 6.76E−56 |
| APEH | 7.12E−05 | 1 | 5674.05193 | −0.5730579 | 1.54E−57 | 8.43E−56 |
| DYNLL2 | 8.62E−05 | 1 | 3745.37105 | 0.64027316 | 1.91E−57 | 1.04E−55 |
| NRG1 | 0.00057089 | 1 | 2660.87726 | 0.81632309 | 2.06E−57 | 1.12E−55 |
| S100A10 | 0.00784637 | 1 | 18964.2931 | 0.55335679 | 3.03E−57 | 1.64E−55 |
| SPRY1 | 0.00039092 | 1 | 1068.13388 | −1.0673344 | 6.98E−57 | 3.76E−55 |
| SNORC | 0.0024312 | 1 | 410.309023 | 1.59345185 | 7.39E−57 | 3.98E−55 |
| LGALS3BP | 0.00317674 | 1 | 42868.6768 | 0.48046325 | 7.61E−57 | 4.08E−55 |
| SLC22A23 | 0.00109293 | 1 | 1606.87279 | −0.9559767 | 7.66E−57 | 4.10E−55 |
| DBN1 | 0.00021951 | 1 | 6849.82098 | 0.50966328 | 7.86E−57 | 4.19E−55 |
| CDKN2A | 8.66E−05 | 1 | 1625.18466 | 0.92263945 | 1.27E−56 | 6.73E−55 |
| B4GALT5 | 0.00136633 | 1 | 6625.82503 | 0.52955804 | 1.43E−56 | 7.60E−55 |
| FAM83H | 0.000238 | 1 | 5397.6502 | 0.53545736 | 1.93E−56 | 1.02E−54 |
| TIAM1 | 0.00037843 | 1 | 884.212141 | −1.1335249 | 2.30E−56 | 1.21E−54 |
| TPRA1 | 6.16E−05 | 1 | 1899.84735 | 0.83760314 | 2.35E−56 | 1.23E−54 |
| PHC2 | 0.00019905 | 1 | 3743.90872 | −0.6197028 | 2.61E−56 | 1.37E−54 |
| TPBG | 0.00453015 | 1 | 3545.98321 | −0.6870446 | 2.95E−56 | 1.54E−54 |
| IGFBP3 | 0.00360832 | 1 | 15093.0764 | 1.30996431 | 2.97E−56 | 1.55E−54 |
| PXDN | 4.53E−05 | 1 | 26656.1095 | −0.4420401 | 3.53E−56 | 1.84E−54 |
| EDEM2 | 5.45E−06 | 0.17539553 | 2702.0793 | 0.66855679 | 6.80E−56 | 3.52E−54 |
| ANKLE2 | 0.00010947 | 1 | 4011.29491 | 0.57536347 | 7.92E−56 | 4.09E−54 |
| EHD2 | 1.92E−05 | 0.61788328 | 7924.10231 | 0.5004918 | 1.08E−55 | 5.59E−54 |
| HIST1H1C | 0.00149713 | 1 | 4064.00868 | 0.83912995 | 1.77E−55 | 9.09E−54 |
| EDN1 | 0.00087397 | 1 | 801.024808 | 1.13337069 | 2.22E−55 | 1.14E−53 |
| B4GALT1 | 0.00450845 | 1 | 9590.39109 | 0.52498419 | 2.88E−55 | 1.47E−53 |
| PTGS2 | 0.00048519 | 1 | 438.592865 | −1.5377815 | 4.92E−55 | 2.51E−53 |
| RTL8C | 0.00304897 | 1 | 6705.15592 | 0.53369009 | 5.80E−55 | 2.95E−53 |
| KCTD15 | 2.74E−06 | 0.08823881 | 1496.73025 | 0.8281767 | 8.59E−55 | 4.35E−53 |
| TSKU | 0.00718685 | 1 | 3602.17101 | 0.78869296 | 9.08E−55 | 4.59E−53 |
| PLXNB1 | 2.29E−05 | 0.73596735 | 1061.63125 | 0.96149202 | 1.16E−54 | 5.83E−53 |
| VLDLR | 7.33E−05 | 1 | 827.329377 | 1.18337848 | 1.51E−54 | 7.61E−53 |
| GLI1 | 0.00013666 | 1 | 536.516375 | 1.46371587 | 2.24E−54 | 1.12E−52 |
| CUEDC1 | 0.00130957 | 1 | 2670.92351 | 0.70329894 | 3.74E−54 | 1.87E−52 |
| EDEM1 | 0.00028254 | 1 | 3121.92747 | −0.676553 | 3.79E−54 | 1.89E−52 |
| DCLK1 | 0.00119657 | 1 | 166.758115 | −2.896373 | 6.02E−54 | 2.99E−52 |
| ALDH3B1 | 0.0063586 | 1 | 4050.51032 | 0.66934659 | 1.08E−53 | 5.33E−52 |
| TPCN1 | 0.0003673 | 1 | 2846.05113 | −0.6535765 | 1.33E−53 | 6.56E−52 |
| RGL2 | 5.44E−05 | 1 | 1643.11168 | −0.8099288 | 1.44E−53 | 7.08E−52 |
| EPHA2 | 0.00043182 | 1 | 4101.0707 | −0.606295 | 2.46E−53 | 1.21E−51 |
| CITED4 | 0.00260452 | 1 | 2279.94552 | −0.8434149 | 3.88E−53 | 1.90E−51 |
| CXCL1 | 0.00014534 | 1 | 513.506617 | −1.416257 | 6.08E−53 | 2.97E−51 |
| WWC1 | 7.60E−05 | 1 | 4512.55881 | 0.55303082 | 6.87E−53 | 3.35E−51 |
| COL4A5 | 0.00070501 | 1 | 4319.93525 | 0.62831257 | 7.19E−53 | 3.50E−51 |
| HAS3 | 0.00115346 | 1 | 548.436123 | −1.3412038 | 1.04E−52 | 5.07E−51 |
| CD81 | 5.19E−06 | 0.16691227 | 5796.24825 | 0.50636335 | 2.61E−52 | 1.26E−50 |
| FERMT1 | 2.02E−05 | 0.64968456 | 1471.48205 | −0.8145841 | 3.22E−52 | 1.55E−50 |
| TSPAN5 | 0.00097378 | 1 | 1260.74024 | −0.8865379 | 6.11E−52 | 2.94E−50 |
| MVD | 0.00027689 | 1 | 3937.31589 | 0.63910298 | 7.55E−52 | 3.63E−50 |
| NECTIN2 | 0.00010621 | 1 | 3852.17724 | 0.59131562 | 7.84E−52 | 3.76E−50 |
| NPTX1 | 1.37E−05 | 0.44042991 | 334.228759 | −1.741328 | 9.03E−52 | 4.32E−50 |
| NPTXR | 0.00560268 | 1 | 1994.27602 | 0.80070769 | 1.47E−51 | 7.00E−50 |
| WIPF1 | 0.00082028 | 1 | 4125.885 | 0.60208504 | 2.45E−51 | 1.16E−49 |
| PPM1H | 0.00074231 | 1 | 1897.39348 | 0.74674519 | 2.46E−51 | 1.16E−49 |
| SEZ6L2 | 0.00031655 | 1 | 4993.88231 | 0.6271285 | 3.29E−51 | 1.56E−49 |
| NABP1 | 0.00293399 | 1 | 1092.69316 | 0.97491995 | 3.93E−51 | 1.85E−49 |
| DCLK2 | 5.89E−05 | 1 | 1360.34139 | 0.82383066 | 7.82E−51 | 3.68E−49 |
| CHPF | 0.00201078 | 1 | 4365.58254 | 0.61582757 | 7.94E−51 | 3.73E−49 |
| HACD1 | 0.00218407 | 1 | 1127.95927 | 0.92338763 | 8.65E−51 | 4.05E−49 |
| PLS3 | 0.00021718 | 1 | 4529.04938 | 0.42360307 | 9.73E−51 | 4.54E−49 |
| ENTPD6 | 0.00023968 | 1 | 3972.82171 | −0.5520749 | 1.25E−50 | 5.81E−49 |
| STAT3 | 0.00255294 | 1 | 32044.7023 | 0.44962349 | 1.29E−50 | 5.98E−49 |
| FZD2 | 0.00010377 | 1 | 1548.37412 | 0.79197597 | 1.48E−50 | 6.84E−49 |
| SYNPO2 | 0.00127351 | 1 | 1921.33218 | 0.76423664 | 1.67E−50 | 7.72E−49 |
| ZNF281 | 0.00282668 | 1 | 4652.11655 | 0.63142636 | 2.57E−50 | 1.19E−48 |
| USP2 | 1.56E−06 | 0.05030344 | 623.803725 | 1.17230492 | 4.80E−50 | 2.21E−48 |
| RAB27B | 0.00048815 | 1 | 915.318649 | −1.0409317 | 7.49E−50 | 3.44E−48 |
| SLC16A14 | 0.00043687 | 1 | 2388.25091 | −0.7451779 | 1.55E−49 | 7.10E−48 |
| HLA-F | 8.20E−05 | 1 | 1047.56604 | 0.9914108 | 1.66E−49 | 7.57E−48 |
| CIB1 | 0.0005659 | 1 | 3965.21643 | −0.5887308 | 1.99E−49 | 9.07E−48 |
| GLIS2 | 0.00121863 | 1 | 1668.7455 | 0.79214445 | 2.90E−49 | 1.32E−47 |
| ANXA3 | 0.00142346 | 1 | 4136.45797 | 0.64359562 | 3.10E−49 | 1.41E−47 |
| PLBD2 | 0.00217248 | 1 | 5008.16168 | 0.54816404 | 3.62E−49 | 1.64E−47 |
| CHP1 | 0.00649082 | 1 | 10930.3299 | 0.52242381 | 5.12E−49 | 2.31E−47 |
| FADS3 | 0.00025315 | 1 | 1522.50224 | 0.79314907 | 5.50E−49 | 2.47E−47 |
| LAMC2 | 0.00140891 | 1 | 4203.10699 | −0.5772839 | 5.81E−49 | 2.61E−47 |
| NAV3 | 0.00016082 | 1 | 1188.43427 | −0.8883126 | 5.90E−49 | 2.64E−47 |
| PARD3B | 4.42E−05 | 1 | 776.900703 | 1.09127377 | 8.26E−49 | 3.69E−47 |
| EPHX1 | 0.00398262 | 1 | 2638.11458 | 0.73178068 | 9.62E−49 | 4.29E−47 |
| IQCD | 1.43E−05 | 0.46118313 | 633.393815 | −1.1833823 | 1.04E−48 | 4.63E−47 |
| COL4A2 | 0.00420291 | 1 | 440.30151 | 1.46064285 | 1.74E−48 | 7.71E−47 |
| TPP1 | 0.00690471 | 1 | 4757.89509 | 0.5930303 | 2.01E−48 | 8.89E−47 |
| RRP1B | 0.00316117 | 1 | 5171.43098 | 0.57500972 | 2.42E−48 | 1.07E−46 |
| HES4 | 1.56E−05 | 0.50126962 | 446.119103 | 1.40239962 | 2.73E−48 | 1.20E−46 |
| NR4A2 | 0.00123434 | 1 | 3201.53117 | −0.7274217 | 3.39E−48 | 1.49E−46 |
| ZMYND8 | 6.89E−06 | 0.22160819 | 3644.83936 | −0.556911 | 3.94E−48 | 1.73E−46 |
| VWA5A | 3.79E−05 | 1 | 1837.14243 | −0.7540515 | 4.11E−48 | 1.80E−46 |
| HYAL3 | 0.00022557 | 1 | 1225.77048 | 0.89926211 | 5.68E−48 | 2.48E−46 |
| MIR100HG | 0.00030659 | 1 | 480.477218 | −1.3730128 | 9.56E−48 | 4.16E−46 |
| ADAM15 | 0.00071878 | 1 | 6856.68678 | 0.46290908 | 1.28E−47 | 5.54E−46 |
| C6orf89 | 0.00082365 | 1 | 3812.35638 | 0.56836577 | 2.27E−47 | 9.83E−46 |
| TSPAN14 | 0.00010732 | 1 | 5974.34918 | −0.5083194 | 2.49E−47 | 1.08E−45 |
| SLC6A6 | 0.00053443 | 1 | 6014.3224 | −0.6083566 | 3.00E−47 | 1.29E−45 |
| WDR1 | 0.00056368 | 1 | 9554.95057 | 0.47656026 | 3.64E−47 | 1.57E−45 |
| ITGA10 | 0.00939065 | 1 | 559.864876 | −2.9052108 | 4.66E−47 | 2.00E−45 |
| PTPN21 | 9.58E−05 | 1 | 1118.03624 | 0.86956358 | 7.08E−47 | 3.03E−45 |
| PDLIM7 | 0.01010151 | 1 | 2300.59984 | 0.77339339 | 7.90E−47 | 3.38E−45 |
| RAPGEF1 | 4.09E−05 | 1 | 4199.15331 | 0.54645574 | 8.87E−47 | 3.78E−45 |
| CYB5B | 1.55E−05 | 0.49764448 | 11563.0101 | −0.4098279 | 2.73E−46 | 1.16E−44 |
| EFEMP1 | 0.00011538 | 1 | 9595.27679 | 0.44929808 | 5.12E−46 | 2.17E−44 |
| PPP1R9B | 0.00011 | 1 | 2718.48687 | −0.5988106 | 8.06E−46 | 3.41E−44 |
| NTN4 | 0.00087532 | 1 | 15620.9943 | 0.40689413 | 1.03E−45 | 4.33E−44 |
| ERG28 | 0.00612648 | 1 | 6419.75641 | 0.56221404 | 1.27E−45 | 5.36E−44 |
| OLMALINC | 0.00067088 | 1 | 780.146669 | −1.0587749 | 1.28E−45 | 5.37E−44 |
| ASAP1 | 0.00034381 | 1 | 7478.78781 | −0.4665544 | 1.65E−45 | 6.92E−44 |
| RICTOR | 3.43E−05 | 1 | 1824.05546 | −0.7178441 | 1.75E−45 | 7.31E−44 |
| GALNS | 3.67E−05 | 1 | 3112.01784 | 0.58812602 | 1.85E−45 | 7.72E−44 |
| VWA7 | 0.0001748 | 1 | 1232.42233 | 0.93256552 | 1.85E−45 | 7.72E−44 |
| ELOVL5 | 0.00082388 | 1 | 11203.194 | 0.45045253 | 4.16E−45 | 1.73E−43 |
| SLC66A3 | 0.0016365 | 1 | 1261.84769 | 0.84605966 | 4.64E−45 | 1.92E−43 |
| ANTXR2 | 0.00016782 | 1 | 881.552022 | −1.0591766 | 4.94E−45 | 2.05E−43 |
| FURIN | 0.00016835 | 1 | 6414.79478 | −0.4725191 | 5.02E−45 | 2.07E−43 |
| TCP11L1 | 5.15E−05 | 1 | 1161.20253 | 0.87565687 | 5.33E−45 | 2.20E−43 |
| COL4A1 | 0.01246112 | 1 | 301.997247 | 1.85503275 | 9.38E−45 | 3.85E−43 |
| GPR155 | 9.36E−05 | 1 | 1178.52536 | 0.89748386 | 9.40E−45 | 3.86E−43 |
| ABTB2 | 0.00010859 | 1 | 1061.12374 | 0.95316177 | 1.07E−44 | 4.38E−43 |
| ZFYVE19 | 0.01262182 | 1 | 2431.58719 | −0.8093612 | 1.11E−44 | 4.53E−43 |
| TBC1D2 | 0.0004853 | 1 | 782.41891 | 0.98815959 | 1.27E−44 | 5.18E−43 |
| PHLDA1 | 0.00132646 | 1 | 1034.42213 | −1.02694 | 1.75E−44 | 7.11E−43 |
| ZMIZ2 | 0.00013353 | 1 | 2766.28119 | 0.59190985 | 4.76E−44 | 1.93E−42 |
| LDHA | 0.00014802 | 1 | 97579.7502 | −0.36262 | 5.75E−44 | 2.33E−42 |
| CEACAM1 | 0.00286661 | 1 | 110.301361 | −4.0933127 | 6.05E−44 | 2.44E−42 |
| CYB5R3 | 0.00184368 | 1 | 8041.843 | 0.46447906 | 6.62E−44 | 2.67E−42 |
| GLI2 | 0.00170116 | 1 | 455.455509 | 1.31220971 | 7.21E−44 | 2.90E−42 |
| SLC20A1 | 0.00071213 | 1 | 5299.90458 | −0.5160387 | 7.94E−44 | 3.19E−42 |
| CTH | 0.00012289 | 1 | 1592.78757 | −0.7967165 | 8.41E−44 | 3.37E−42 |
| POMP | 2.20E−05 | 0.70599542 | 2305.90638 | −0.6302214 | 1.05E−43 | 4.20E−42 |
| TSC22D1 | 0.00012363 | 1 | 5958.37706 | −0.5089421 | 1.36E−43 | 5.43E−42 |
| SPTAN1 | 0.00037446 | 1 | 16813.4901 | 0.38419416 | 2.16E−43 | 8.60E−42 |
| FGB | 9.88E−05 | 1 | 58658.6237 | 0.36819033 | 3.48E−43 | 1.38E−41 |
| IGSF9 | 0.00013436 | 1 | 781.241987 | 1.03888347 | 3.57E−43 | 1.41E−41 |
| NXPH3 | 0.00120521 | 1 | 175.338486 | 2.1632897 | 3.97E−43 | 1.57E−41 |
| PLXNA1 | 0.00102134 | 1 | 3303.0363 | 0.56949668 | 4.78E−43 | 1.88E−41 |
| STC2 | 0.00027902 | 1 | 5865.08908 | −0.5567889 | 4.86E−43 | 1.91E−41 |
| HK2 | 0.00215177 | 1 | 2253.55348 | −0.8052077 | 4.90E−43 | 1.92E−41 |
| SLAIN1 | 1.04E−06 | 0.03360075 | 989.841056 | −0.8792192 | 4.90E−43 | 1.92E−41 |
| RHOA | 0.00047513 | 1 | 23016.3011 | 0.39290554 | 7.95E−43 | 3.11E−41 |
| PRKACB | 0.00256601 | 1 | 2194.10078 | −0.6551607 | 8.67E−43 | 3.38E−41 |
| ZNFX1 | 0.00508036 | 1 | 3423.0572 | 0.63174515 | 1.55E−42 | 6.03E−41 |
| SGPL1 | 0.00316155 | 1 | 5318.90531 | 0.4988806 | 1.64E−42 | 6.36E−41 |
| MYO1C | 0.00044488 | 1 | 21274.2316 | 0.39627547 | 1.86E−42 | 7.21E−41 |
| PKM | 3.87E−05 | 1 | 95877.4271 | −0.3334693 | 2.19E−42 | 8.47E−41 |
| C1QL1 | 0.00540151 | 1 | 764.310396 | 1.0277387 | 2.43E−42 | 9.40E−41 |
| IFNAR1 | 0.0002076 | 1 | 3864.8405 | 0.50500374 | 2.62E−42 | 1.01E−40 |
| STN1 | 0.00155972 | 1 | 1133.14982 | −0.951678 | 3.28E−42 | 1.26E−40 |
| APOE | 0.00060313 | 1 | 1834.48618 | 1.48824238 | 3.64E−42 | 1.40E−40 |
| TYMP | 0.00317554 | 1 | 1231.79673 | 0.85483381 | 4.28E−42 | 1.64E−40 |
| EPHA10 | 0.00041153 | 1 | 603.270078 | 1.09298267 | 5.63E−42 | 2.15E−40 |
| SLC41A2 | 0.00034641 | 1 | 664.379612 | 1.02920292 | 6.54E−42 | 2.49E−40 |
| SOWAHD | 1.99E−05 | 0.64061462 | 126.284275 | 2.72805543 | 9.66E−42 | 3.68E−40 |
| FOCAD | 0.00070361 | 1 | 4126.80724 | −0.6528107 | 1.01E−41 | 3.82E−40 |
| AKAP5 | 0.00090161 | 1 | 710.49621 | 1.01110973 | 1.11E−41 | 4.22E−40 |
| SSTR1 | 0.00237936 | 1 | 241.14996 | −1.8727249 | 1.12E−41 | 4.24E−40 |
| SEC61A1 | 0.00031949 | 1 | 13421.5741 | 0.42230975 | 1.17E−41 | 4.43E−40 |
| PLSCR1 | 2.35E−05 | 0.75584791 | 989.948618 | −0.8711545 | 1.28E−41 | 4.81E−40 |
| TLE1 | 4.93E−05 | 1 | 1556.6402 | 0.71906355 | 1.52E−41 | 5.72E−40 |
| MAFF | 0.00225188 | 1 | 674.520183 | −1.156674 | 1.58E−41 | 5.92E−40 |
| PPIL3 | 0.00087363 | 1 | 921.33356 | −0.8954264 | 1.61E−41 | 6.04E−40 |
| TRPC4 | 0.00075305 | 1 | 874.38688 | 0.91789174 | 1.76E−41 | 6.58E−40 |
| PVR | 0.00141085 | 1 | 4456.73152 | 0.51975368 | 3.86E−41 | 1.44E−39 |
| OBSL1 | 0.00198154 | 1 | 1281.82059 | 0.8108773 | 3.87E−41 | 1.44E−39 |
| SOWAHC | 0.00064986 | 1 | 2010.96621 | −0.7106447 | 5.19E−41 | 1.93E−39 |
| BLVRB | 4.02E−05 | 1 | 5065.8321 | 0.47738101 | 5.78E−41 | 2.14E−39 |
| RAB15 | 0.00074366 | 1 | 1947.85741 | 0.64551022 | 6.25E−41 | 2.31E−39 |
| ATP11A | 4.02E−05 | 1 | 8245.62163 | −0.4278748 | 6.27E−41 | 2.32E−39 |
| AADACP1 | 3.11E−05 | 0.99835365 | 156.318322 | −2.3549943 | 1.00E−40 | 3.70E−39 |
| MAP3K5 | 3.10E−05 | 0.99428893 | 1817.67249 | −0.6614326 | 1.14E−40 | 4.18E−39 |
| WFS1 | 0.00167594 | 1 | 2909.71808 | 0.57100202 | 1.74E−40 | 6.40E−39 |
| CREG1 | 0.00197589 | 1 | 5537.28625 | 0.45392007 | 1.80E−40 | 6.59E−39 |
| IGFBP1 | 0.00365471 | 1 | 104.950562 | −4.702528 | 1.87E−40 | 6.85E−39 |
| HNRNPA2B1 | 0.00441456 | 1 | 33045.9052 | −0.4176168 | 2.18E−40 | 7.94E−39 |
| CTXN1 | 0.00319387 | 1 | 521.862781 | 1.29047921 | 2.36E−40 | 8.61E−39 |
| AADAC | 0.00058933 | 1 | 183.338334 | −2.3414811 | 2.50E−40 | 9.09E−39 |
| LRRC8A | 7.56E−05 | 1 | 1746.94883 | 0.7037871 | 2.52E−40 | 9.14E−39 |
| TTC9 | 0.00259409 | 1 | 900.419053 | 0.91488768 | 2.84E−40 | 1.03E−38 |
| ITGB2 | 0.00019493 | 1 | 1188.30546 | −0.8004893 | 2.89E−40 | 1.04E−38 |
| TNNC1 | 0.00070057 | 1 | 88.1516935 | 3.72094466 | 3.11E−40 | 1.12E−38 |
| TRAF3 | 0.00017578 | 1 | 4267.35558 | 0.54634124 | 4.89E−40 | 1.76E−38 |
| OS9 | 0.00063945 | 1 | 6032.95565 | 0.42907599 | 5.85E−40 | 2.10E−38 |
| VCL | 0.00045713 | 1 | 17732.8658 | 0.39654753 | 6.34E−40 | 2.27E−38 |
| TNESF10 | 5.25E−05 | 1 | 658.963433 | −1.0495389 | 7.83E−40 | 2.80E−38 |
| CLIC1 | 0.00290701 | 1 | 19823.7049 | −0.3716308 | 9.07E−40 | 3.24E−38 |
| CDR2L | 0.00085315 | 1 | 1372.94514 | 0.72825457 | 1.07E−39 | 3.82E−38 |
| DIO3 | 0.00023652 | 1 | 511.544056 | 1.21584813 | 1.11E−39 | 3.95E−38 |
| TENT5B | 0.00203033 | 1 | 770.970307 | 0.99948289 | 1.12E−39 | 3.99E−38 |
| TPM4 | 0.00029092 | 1 | 44228.8451 | 0.3681853 | 1.17E−39 | 4.15E−38 |
| CRLF2 | 0.0003226 | 1 | 140.671214 | −2.549054 | 1.30E−39 | 4.58E−38 |
| CCDC102A | 0.000117 | 1 | 1074.63433 | 0.85287842 | 1.30E−39 | 4.60E−38 |
| GPX1 | 0.00076195 | 1 | 5301.41538 | −0.5423042 | 1.50E−39 | 5.29E−38 |
| MIDN | 6.06E−05 | 1 | 4102.22347 | −0.5101531 | 1.51E−39 | 5.29E−38 |
| CELSR2 | 0.0032143 | 1 | 2339.10408 | 0.68597476 | 1.52E−39 | 5.34E−38 |

Annotated RHO pathway genes for H2122 (top) and H2030 (bottom). (Bold =
RHO genes and RHO regulators. Italic = Actin and myosin genes and regulators.
Underline = Integrin activator. Bold and underlined = ROCK (Rho Kinase) targets.)

| row | ctrl | MRTX | log2FoldChange.unshrunken | FDR |
|---|---|---|---|---|
| AKAP13 | 3669.5242 | 5369.45933 | 0.54918381 | 6.53E−39 |
| ALDH3A2 | 7879.42401 | 9962.73631 | 0.33845187 | 7.54E−21 |
| AMIGO2 | 2578.39192 | 2940.40882 | 0.18954518 | 0.00585456 |
| ANKFY1 | 1379.4663 | 1894.14183 | 0.45743414 | 7.86E−15 |
| ANKLE2 | 4772.79552 | 5229.47975 | 0.1318329 | 0.00329318 |
| ARHGAP17 | 1193.18468 | 1405.71596 | 0.23648775 | 0.00159665 |
| ARHGAP21 | 6709.7134 | 8944.2047 | 0.41470206 | 1.09E−24 |
| ARHGAP23 | 567.643297 | 1019.65543 | 0.84502516 | 3.04E−26 |
| ARHGAP42 | 829.781585 | 1274.29092 | 0.61889113 | 4.13E−17 |
| ARHGEF10L | 642.367442 | 803.846784 | 0.32352177 | 0.00058241 |
| ARHGEF17 | 1302.69704 | 1937.30912 | 0.57255256 | 6.22E−22 |
| ARHGEF7 | 2228.75027 | 2450.56189 | 0.13687761 | 0.01766966 |
| ARPC5 | 5157.46263 | 6755.88414 | 0.38948312 | 4.98E−19 |
| ATP6AP1 | 2513.63597 | 3111.32602 | 0.30775385 | 2.06E−10 |
| BAIAP2 | 2162.72266 | 2429.36562 | 0.16773096 | 0.00250143 |
| BASP1 | 10155.8723 | 16967.5627 | 0.74046518 | 2.46E−14 |
| CAV1 | 2907.86116 | 3900.65238 | 0.42375705 | 3.38E−12 |
| CCDC115 | 473.157086 | 665.198953 | 0.49146667 | 1.36E−07 |
| CDC42BPB | 4670.27392 | 6138.57481 | 0.39439658 | 3.56E−25 |
| CDC42EP1 | 2159.19164 | 2385.5917 | 0.14385585 | 0.01967322 |
| CENPT | 311.262799 | 400.3252 | 0.36303928 | 0.00281964 |
| CTTN | 9142.83168 | 10656.7998 | 0.2210613 | 4.95E−10 |
| DAAM1 | 950.969429 | 1218.58476 | 0.35773573 | 5.81E−07 |
| DLG5 | 3508.60086 | 4280.71974 | 0.28695755 | 2.23E−11 |
| DOCK1 | 1233.37174 | 1376.68131 | 0.15858693 | 0.04838802 |
| DSG2 | 12258.973 | 13728.0219 | 0.16328564 | 8.71E−07 |
| DYNLL2 | 5457.26914 | 5929.81169 | 0.1198071 | 0.00985232 |
| ERBIN | 4518.96838 | 5084.79559 | 0.17019632 | 0.00231123 |
| FAM13B | 875.18936 | 1569.14862 | 0.84231489 | 8.40E−37 |
| FARP2 | 1076.42043 | 1344.82308 | 0.32117472 | 1.02E−06 |
| FERMT2 | 1752.57408 | 2286.03801 | 0.38337396 | 1.64E−12 |
| FNBP1L | 1788.41002 | 2457.82399 | 0.45870407 | 4.08E−17 |
| GIT1 | 2762.72816 | 3323.96653 | 0.26681224 | 1.57E−08 |
| GOLGA3 | 4256.11187 | 4661.97451 | 0.13140504 | 0.0146759 |
| GOPC | 1035.1488 | 1269.82427 | 0.29479069 | 2.91E−05 |
| IL32 | 2319.8699 | 3088.21551 | 0.41272954 | 4.34E−13 |
| IQGAP3 | 3380.36108 | 4208.12988 | 0.31600187 | 3.76E−10 |
| ITSN1 | 1462.03432 | 1709.56588 | 0.22565284 | 0.00085451 |
| KIDINS220 | 2424.13985 | 2727.88979 | 0.17031243 | 0.00140046 |
| KTN1 | 13986.096 | 15783.6828 | 0.17444055 | 8.04E−07 |
| LIMK2 | 1351.18854 | 1721.64386 | 0.34955774 | 7.10E−08 |
| LLGL1 | 1481.50911 | 1664.60032 | 0.16810832 | 0.0194432 |
| MAPK1 | 5394.62697 | 5750.82813 | 0.09224652 | 0.03493808 |
| MCAM | 255.712413 | 970.846697 | 1.92472131 | 3.39E−91 |
| MRTFA | 685.552549 | 1025.08534 | 0.58040486 | 2.94E−14 |
| MYH10 | 3211.56442 | 3557.9361 | 0.14776436 | 0.00329693 |
| MYH14 | 4649.21316 | 11935.5451 | 1.36020597 | 4.05E−201 |
| MYH9 | 23086.4598 | 29613.9766 | 0.35923127 | 3.89E−18 |
| MYL12B | 12426.2393 | 13190.0887 | 0.08606452 | 0.0321205 |
| MYL6 | 10991.6891 | 14689.4575 | 0.41836802 | 1.44E−19 |
| MYL9 | 71.1138258 | 416.430121 | 2.54987245 | 3.58E−64 |
| MYLK | 23.2505751 | 43.4270089 | 0.90132619 | 0.02404863 |
| MYO9B | 1453.12654 | 1632.19578 | 0.16765378 | 0.01413869 |
| NCF2 | 772.452603 | 2123.65571 | 1.45903157 | 9.86E−129 |
| NCK2 | 546.670848 | 650.257519 | 0.25033873 | 0.01481598 |
| NCOA2 | 1288.26949 | 1661.84026 | 0.3673473 | 3.39E−08 |
| PGRMC2 | 1752.4865 | 2524.10673 | 0.52636958 | 1.32E−22 |
| PICALM | 9048.61075 | 10294.8265 | 0.1861513 | 8.50E−07 |
| PIK3R3 | 219.605566 | 384.317973 | 0.80738583 | 2.24E−11 |
| PLEKHG4 | 1562.59827 | 1710.11854 | 0.13014941 | 0.04979607 |
| PLXNA1 | 3503.07804 | 3945.19661 | 0.17147407 | 0.0005574 |
| PLXNB1 | 1071.83161 | 1708.0602 | 0.67228056 | 5.83E−29 |
| PRKCD | 1570.70709 | 2313.88345 | 0.55890203 | 8.14E−25 |
| PRKCZ | 330.127943 | 458.360271 | 0.47345675 | 9.58E−05 |
| PTPN13 | 570.250844 | 687.552394 | 0.26987298 | 0.00308394 |
| RAPGEF1 | 3350.25272 | 3715.70132 | 0.14936461 | 0.00165997 |
| RCC2 | 4909.77475 | 5571.95917 | 0.18252785 | 8.84E−05 |
| RHOA | 17045.9805 | 20387.4718 | 0.25825129 | 1.19E−14 |
| RHOB | 2665.23116 | 4234.20773 | 0.66783138 | 1.43E−38 |
| RHOD | 3177.11664 | 3861.11601 | 0.28129984 | 1.54E−07 |
| RHPN1 | 109.22235 | 151.930255 | 0.47614109 | 0.03123448 |
| RNF20 | 1806.34732 | 2058.25503 | 0.18834644 | 0.00213196 |
| SCRIB | 4493.3176 | 5222.28546 | 0.21690028 | 0.00063495 |
| SHKBP1 | 1488.64029 | 1645.77377 | 0.14477085 | 0.04265469 |
| SPTAN1 | 12832.8259 | 16795.6842 | 0.38825167 | 1.01E−25 |
| SRF | 1165.67254 | 1304.0298 | 0.16181428 | 0.04086451 |
| STK38 | 1362.26504 | 2004.36495 | 0.55713779 | 1.20E−21 |
| SWAP70 | 1931.10486 | 2358.57412 | 0.28848843 | 4.00E−08 |
| SYDE2 | 245.401947 | 359.302389 | 0.55005183 | 2.10E−05 |
| TMEM59 | 2783.20668 | 4185.58468 | 0.58868112 | 1.93E−36 |
| TRIO | 3658.25404 | 4717.39439 | 0.36683496 | 5.15E−16 |
| VAV2 | 3169.18835 | 3437.979 | 0.11744733 | 0.03260845 |
| WDR11 | 2436.60615 | 2698.27166 | 0.14716253 | 0.00561824 |
| WDR6 | 2982.67591 | 3375.25574 | 0.17838959 | 0.00141342 |
| WIPF1 | 579.606958 | 757.7961 | 0.3867348 | 1.88E−05 |
| YWHAG | 34023.9791 | 36758.8275 | 0.11153887 | 0.00414293 |
| YWHAH | 2978.19048 | 3594.03178 | 0.27116714 | 2.01E−07 |
| AKAP13 | 3016.69913 | 3932.8581 | 0.38260732 | 7.83E−12 |
| ALDH3A2 | 5858.44141 | 6381.74151 | 0.12343327 | 0.0011958 |
| AMIGO2 | 4515.31589 | 5671.56661 | 0.32892037 | 4.96E−16 |
| ANKFY1 | 2084.5969 | 2708.0336 | 0.3774772 | 6.47E−16 |
| ANKLE2 | 3222.50896 | 4800.08085 | 0.57487434 | 4.09E−54 |
| ARHGAP17 | 3377.51015 | 4680.84843 | 0.47080995 | 4.09E−34 |
| ARHGAP21 | 3527.92222 | 3918.89237 | 0.15162719 | 0.00080132 |
| ARHGAP23 | 298.766441 | 432.293875 | 0.53299429 | 8.94E−07 |
| ARHGAP42 | 376.413183 | 722.972277 | 0.94162317 | 3.23E−28 |
| ARHGEF10L | 514.600418 | 742.984944 | 0.52988035 | 1.88E−09 |
| ARHGEF17 | 1488.20757 | 2646.77475 | 0.83065965 | 1.54E−69 |
| ARHGEF7 | 1019.21311 | 1127.30159 | 0.14541779 | 0.04864881 |
| ARPC5 | 6036.78448 | 7959.30585 | 0.39886232 | 2.51E−27 |
| ATP6AP1 | 5848.34375 | 7405.7974 | 0.34062697 | 2.28E−24 |
| BAIAP2 | 463.816919 | 607.098108 | 0.38837423 | 2.84E−05 |
| BASP1 | 23621.0143 | 35276.1027 | 0.57862027 | 4.31E−97 |
| CAV1 | 12290.601 | 27506.4541 | 1.16221471 | 4.91E−223 |
| CCDC115 | 1684.66065 | 1899.27611 | 0.17299164 | 0.00264118 |
| CDC42BPB | 9947.98785 | 11142.3392 | 0.16357549 | 2.16E−07 |
| CDC42EP1 | 2077.12693 | 2656.22233 | 0.35478653 | 3.55E−11 |
| CENPT | 1158.00437 | 1753.22245 | 0.59836836 | 1.54E−22 |
| CTTN | 7408.02946 | 8982.13337 | 0.27796831 | 4.97E−19 |
| DAAM1 | 1084.7548 | 1390.54412 | 0.35828055 | 8.63E−09 |
| DLG5 | 2228.10332 | 3003.38229 | 0.43077199 | 6.10E−17 |
| DOCK1 | 3382.97127 | 4076.24338 | 0.26894927 | 1.28E−11 |
| DSG2 | 4371.6449 | 5119.3847 | 0.2277942 | 2.14E−08 |
| DYNLL2 | 2928.69873 | 4562.04338 | 0.63942037 | 1.04E−55 |
| ERBIN | 8991.21395 | 10276.9217 | 0.19282037 | 1.67E−08 |
| FAM13B | 869.489732 | 1205.30827 | 0.47116128 | 9.26E−13 |
| FARP2 | 1185.92333 | 1472.12095 | 0.31188546 | 1.81E−07 |
| FERMT2 | 5806.33367 | 6651.58672 | 0.19607105 | 7.78E−07 |
| FNBP1L | 3874.7642 | 5277.78742 | 0.44582472 | 3.99E−34 |
| GIT1 | 1447.66886 | 1805.81315 | 0.31891699 | 3.41E−09 |
| GOLGA3 | 3080.10912 | 4063.17313 | 0.39962538 | 1.21E−22 |
| GOPC | 1994.23187 | 2585.38334 | 0.37454505 | 1.21E−10 |
| IL32 | 25.4280438 | 171.090085 | 2.75026377 | 1.29E−31 |
| IQGAP3 | 6295.81224 | 7603.1145 | 0.272198 | 7.02E−11 |
| ITSN1 | 2520.42985 | 2993.87009 | 0.24834182 | 3.54E−08 |
| KIDINS220 | 2396.06346 | 2874.91775 | 0.26285456 | 7.02E−09 |
| KTN1 | 29683.3716 | 34229.0531 | 0.20556641 | 2.06E−12 |
| LIMK2 | 705.184966 | 876.280094 | 0.31339037 | 6.37E−05 |
| LLGL | 1830.57094 | 2083.47391 | 0.18669735 | 0.0003503 |
| MAPK1 | 5247.31233 | 5592.25229 | 0.09185078 | 0.04240568 |
| MCAM | 127.149554 | 277.362929 | 1.12524857 | 1.29E−14 |
| MRTFA | 2618.36257 | 4490.29029 | 0.77814383 | 2.59E−73 |
| MYH10 | 8685.5683 | 9415.93366 | 0.11648391 | 0.00086466 |
| MYH14 | 668.732444 | 1042.17633 | 0.64009837 | 3.51E−17 |
| MYH9 | 42583.5244 | 70266.9061 | 0.72255002 | 1.37E−132 |
| MYL12B | 15965.2214 | 17466.3641 | 0.12964676 | 2.83E−05 |
| MYL6 | 13111.9293 | 19990.1917 | 0.60841233 | 5.61E−106 |
| MYL9 | 9191.01802 | 26367.5483 | 1.52046686 | 0 |
| MYLK | 1028.56735 | 3576.86487 | 1.79805935 | 3.43E−303 |
| MYO9B | 2310.91501 | 2825.41199 | 0.28999705 | 1.22E−09 |
| NCF2 | 9.30995962 | 74.9295475 | 3.00868792 | 1.98E−15 |
| NCK2 | 990.686906 | 1310.99652 | 0.40416276 | 7.30E−10 |
| NCOA2 | 2152.8284 | 2330.87954 | 0.11464112 | 0.04041371 |
| PGRMC2 | 2438.13463 | 3769.17234 | 0.62846997 | 5.86E−37 |
| PICALM | 4749.73045 | 5049.48129 | 0.08828955 | 0.04757416 |
| PIK3R3 | 747.573696 | 1101.4236 | 0.55908171 | 4.81E−15 |
| PLEKHG4 | 2916.02012 | 3137.22562 | 0.10548861 | 0.02600333 |
| PLXNA1 | 2660.0071 | 3946.0655 | 0.56898481 | 1.88E−41 |
| PLXNB1 | 720.336484 | 1402.92602 | 0.96169605 | 5.83E−53 |
| PRKCD | 591.786237 | 936.963647 | 0.66291693 | 2.34E−18 |
| PRKCZ | 571.820871 | 742.237167 | 0.37631697 | 4.24E−06 |
| PTPN13 | 598.205636 | 855.074768 | 0.51540907 | 8.45E−11 |
| RAPGEF1 | 3412.68027 | 4985.62635 | 0.54686951 | 3.78E−45 |
| RCC2 | 5648.70099 | 6019.29005 | 0.0916742 | 0.03291176 |
| RHOA | 19898.9868 | 26133.6155 | 0.39321176 | 3.11E−41 |
| RHOB | 3296.90775 | 7322.0361 | 1.15113137 | 7.24E−123 |
| RHOD | 1432.07594 | 2245.42558 | 0.64888091 | 2.75E−37 |
| RHPN1 | 324.310846 | 447.874286 | 0.46571657 | 5.27E−05 |
| RNF20 | 1793.30185 | 2297.29799 | 0.35731966 | 1.84E−12 |
| SCRIB | 3576.93537 | 3842.48056 | 0.10331391 | 0.02873705 |
| SHKBP1 | 1713.84206 | 2297.56546 | 0.4228718 | 8.93E−15 |
| SPTAN1 | 14587.358 | 19039.6222 | 0.38428623 | 8.60E−42 |
| SRF | 3147.95062 | 4227.68148 | 0.42545378 | 9.59E−21 |
| STK38 | 3911.49269 | 6183.18861 | 0.66063175 | 5.00E−66 |
| SWAP70 | 1793.76803 | 2155.10038 | 0.26476174 | 2.46E−06 |
| SYDE2 | 413.188802 | 504.092052 | 0.28688605 | 0.00489587 |
| TMEM59 | 3457.89385 | 3874.98254 | 0.16429623 | 0.00019161 |
| TRIO | 6307.18418 | 7943.49278 | 0.33277744 | 4.04E−20 |
| VAV2 | 1031.42501 | 1169.11658 | 0.18077987 | 0.01275737 |
| WDR11 | 2023.52047 | 2446.01453 | 0.27356553 | 9.35E−09 |
| WDR6 | 4019.105 | 4439.84373 | 0.14363463 | 0.00155225 |
| WIPF1 | 3277.76149 | 4974.00851 | 0.6016981 | 1.16E−49 |
| YWHAG | 10084.0657 | 13037.1507 | 0.37055118 | 2.69E−31 |
| YWHAH | 4456.41723 | 6914.85369 | 0.63381442 | 1.37E−75 |

TABLE 4
Synthetic lethal (i.e., dropout) genes (FDR <0.1) from TNO155 CRISPR/Cas9 screens of NSCLC cell lines (MaGeCK analysis).

| | | | | | | | | | | | | | |
|---|---|---|---|---|---|---|---|---|---|---|---|---|---|
| id | num | neg\|score | neg\|p-value | neg\|fdr | neg\|rank | neg\|goodsgrna | neg\|lfc | pos\|score | pos\|p-value | pos\|fdr | pos\|rank | pos\|goodsgrna | pos\|lfc |

H23 TNO SL Gene FDR 0.1

| | | | | | | | | | | | | | |
|---|---|---|---|---|---|---|---|---|---|---|---|---|---|
| RTCB | 4 | 2.89E−10 | 2.74E−07 | 0.000171 | 1 | 4 | −2.834 | 1 | 1 | 1 | 18053 | 0 | −2.834 |
| SOD2 | 4 | 3.19E−10 | 2.74E−07 | 0.000171 | 2 | 4 | −3.2554 | 0.98939 | 0.9895 | 1 | 16742 | 0 | −3.2554 |
| BUB3 | 4 | 1.35E−09 | 2.74E−07 | 0.000171 | 3 | 4 | −3.0165 | 1 | 1 | 1 | 18052 | 0 | −3.0165 |
| COA6 | 4 | 2.13E−09 | 2.74E−07 | 0.000171 | 4 | 4 | −2.6727 | 1 | 1 | 1 | 18051 | 0 | −2.6727 |
| DBR1 | 4 | 3.10E−09 | 2.74E−07 | 0.000171 | 5 | 4 | −3.5382 | 1 | 1 | 1 | 18050 | 0 | −3.5382 |
| RCL1 | 4 | 3.24E−09 | 2.74E−07 | 0.000171 | 6 | 4 | −2.6871 | 1 | 1 | 1 | 18049 | 0 | −2.6871 |
| RNMT | 4 | 4.48E−09 | 2.74E−07 | 0.000171 | 7 | 4 | −2.4992 | 1 | 1 | 1 | 18048 | 0 | −2.4992 |
| ALG1 | 4 | 9.29E−09 | 2.74E−07 | 0.000171 | 8 | 4 | −2.8524 | 1 | 1 | 1 | 18047 | 0 | −2.8524 |
| RNASEH2A | 4 | 1.31E−08 | 2.74E−07 | 0.000171 | 9 | 4 | −2.3229 | 1 | 1 | 1 | 18046 | 0 | −2.322 |
| DKC1 | 4 | 1.82E−08 | 2.74E−07 | 0.000171 | 10 | 4 | −3.1215 | 1 | 1 | 1 | 18045 | 0 | −3.1215 |
| WBSCR16 | 4 | 2.09E−08 | 2.74E−07 | 0.000171 | 11 | 4 | −2.1637 | 1 | 1 | 1 | 18044 | 0 | −2.1637 |
| VHL | 4 | 3.65E−08 | 2.74E−07 | 0.000171 | 12 | 4 | −2.1665 | 1 | 1 | 1 | 18043 | 0 | −2.1665 |
| LYRM4 | 4 | 5.13E−08 | 2.74E−07 | 0.000171 | 13 | 4 | −1.7639 | 1 | 1 | 1 | 18042 | 0 | −1.7639 |
| N6AMT1 | 4 | 5.51E−08 | 2.74E−07 | 0.000171 | 14 | 4 | −1.955 | 1 | 1 | 1 | 18041 | 0 | −1.955 |
| PSMG4 | 4 | 6.72E−08 | 2.74E−07 | 0.000171 | 15 | 3 | −1.1322 | 0.1296 | 0.27233 | 0.900806 | 5399 | 1 | −1.132 |
| IDH3A | 4 | 7.05E−08 | 2.74E−07 | 0.000171 | 16 | 4 | −1.6763 | 1 | 1 | 1 | 18040 | 0 | −1.6763 |
| SELRC1 | 4 | 7.16E−08 | 2.74E−07 | 0.000171 | 17 | 3 | −3.0742 | 0.8648 | 0.8698 | 1 | 13898 | 1 | −3.0742 |
| GTPBP10 | 4 | 9.74E−08 | 2.74E−07 | 0.000171 | 18 | 4 | −3.3754 | 0.99998 | 0.99998 | 1 | 17870 | 0 | −3.3754 |
| SRP14 | 4 | 1.10E−07 | 2.74E−07 | 0.000171 | 19 | 4 | −3.281 | 1 | 1 | 1 | 18039 | 0 | −3.281 |
| SAMM50 | 4 | 1.14E−07 | 2.74E−07 | 0.000171 | 20 | 4 | −2.2247 | 1 | 1 | 1 | 18038 | 0 | −2.2247 |
| GNB1L | 3 | 1.15E−07 | 2.74E−07 | 0.000171 | 21 | 3 | −1.9507 | 1 | 1 | 1 | 18037 | 0 | −1.9507 |
| UTP23 | 4 | 1.29E−07 | 2.74E−07 | 0.000171 | 22 | 4 | −1.8462 | 1 | 1 | 1 | 18036 | 0 | −1.8462 |
| RPUSD3 | 4 | 1.29E−07 | 2.74E−07 | 0.000171 | 23 | 4 | −2.5336 | 1 | 1 | 1 | 18035 | 0 | −2.5336 |
| SPATA5 | 4 | 1.30E−07 | 2.74E−07 | 0.000171 | 24 | 4 | −1.9859 | 1 | 1 | 1 | 18034 | 0 | −1.9859 |
| ARL2 | 4 | 1.33E−07 | 2.74E−07 | 0.000171 | 25 | 4 | −1.8348 | 1 | 1 | 1 | 18033 | 0 | −1.8348 |
| CUL2 | 4 | 1.36E−07 | 2.74E−07 | 0.000171 | 26 | 4 | −2.0873 | 1 | 1 | 1 | 18032 | 0 | −2.0873 |
| MTG2 | 4 | 1.54E−07 | 2.74E−07 | 0.000171 | 27 | 4 | −2.7169 | 1 | 1 | 1 | 18031 | 0 | −2.7169 |
| DDOST | 4 | 1.56E−07 | 2.74E−07 | 0.000171 | 28 | 4 | −1.7125 | 1 | 1 | 1 | 18030 | 0 | −1.7125 |
| PET117 | 4 | 1.70E−07 | 2.74E−07 | 0.000171 | 29 | 4 | −2.8055 | 1 | 1 | 1 | 18029 | 0 | −2.8055 |
| COQ2 | 4 | 1.89E−07 | 8.23E−07 | 0.000391 | 30 | 4 | −1.9433 | 1 | 1 | 1 | 17983 | 0 | −1.9433 |
| DLD | 4 | 2.66E−07 | 8.23E−07 | 0.000391 | 31 | 4 | −1.4522 | 1 | 1 | 1 | 18028 | 0 | −1.4522 |
| SAE1 | 4 | 2.84E−07 | 8.23E−07 | 0.000391 | 32 | 4 | −1.9173 | 1 | 1 | 1 | 18027 | 0 | −1.9173 |
| RABGGTB | 4 | 2.85E−07 | 8.23E−07 | 0.000391 | 33 | 4 | −3.0958 | 1 | 1 | 1 | 18026 | 0 | −3.0958 |
| PRDX1 | 3 | 3.00E−07 | 8.23E−07 | 0.000391 | 34 | 3 | −3.1751 | 1 | 1 | 1 | 18025 | 0 | −3.1751 |
| NAA25 | 4 | 3.03E−07 | 8.23E−07 | 0.000391 | 35 | 4 | −2.5153 | 1 | 1 | 1 | 18024 | 0 | −2.5153 |
| GUK1 | 4 | 3.05E−07 | 8.23E−07 | 0.000391 | 36 | 4 | −2.6418 | 1 | 1 | 1 | 18023 | 0 | −2.6418 |
| TBCE | 4 | 3.47E−07 | 8.23E−07 | 0.000391 | 37 | 4 | −1.4612 | 1 | 1 | 1 | 18022 | 0 | −1.4612 |
| BCS1L | 4 | 3.76E−07 | 8.23E−07 | 0.000391 | 38 | 3 | −2.7405 | 0.79847 | 0.8266 | 1 | 13120 | 1 | −2.7405 |
| PPP4C | 4 | 3.93E−07 | 1.37E−06 | 0.000576 | 39 | 4 | −3.2947 | 1 | 1 | 1 | 18021 | 0 | −3.2947 |
| PGD | 4 | 4.08E−07 | 1.37E−06 | 0.000576 | 40 | 4 | −1.7202 | 1 | 1 | 1 | 18020 | 0 | −1.7202 |
| SCO2 | 4 | 4.27E−07 | 1.37E−06 | 0.000576 | 41 | 4 | −2.2893 | 0.99998 | 0.99998 | 1 | 17897 | 0 | −2.2893 |
| ERCC2 | 4 | 4.45E−07 | 1.37E−06 | 0.000576 | 42 | 4 | −2.1082 | 1 | 1 | 1 | 18019 | 0 | −2.1082 |
| DOLK | 4 | 4.50E−07 | 1.37E−06 | 0.000576 | 43 | 4 | −2.9076 | 1 | 1 | 1 | 18014 | 0 | −2.9076 |
| MCMBP | 4 | 5.16E−07 | 1.92E−06 | 0.000707 | 44 | 4 | −3.0601 | 0.99966 | 0.99968 | 1 | 17629 | 0 | −3.0601 |
| RPN1 | 4 | 5.35E−07 | 1.92E−06 | 0.000707 | 45 | 4 | −1.732 | 1 | 1 | 1 | 18018 | 0 | −1.732 |
| MRPL28 | 4 | 5.67E−07 | 1.92E−06 | 0.000707 | 46 | 4 | −2.874 | 1 | 1 | 1 | 18017 | 0 | −2.874 |
| MTERFD2 | 4 | 5.70E−07 | 1.92E−06 | 0.000707 | 47 | 4 | −1.5209 | 1 | 1 | 1 | 18016 | 0 | −1.5209 |
| ALG2 | 4 | 6.18E−07 | 1.92E−06 | 0.000707 | 48 | 4 | −3.2292 | 1 | 1 | 1 | 18015 | 0 | −3.2292 |
| NAA20 | 4 | 6.72E−07 | 1.92E−06 | 0.000707 | 49 | 4 | −3.0588 | 0.96419 | 0.96412 | 1 | 15847 | 0 | −3.0588 |
| MBTPS2 | 4 | 7.52E−07 | 2.47E−06 | 0.00081 | 50 | 4 | −1.5796 | 0.97787 | 0.97797 | 1 | 16288 | 0 | −1.5796 |
| MRPL53 | 4 | 7.65E−07 | 2.47E−06 | 0.00081 | 51 | 4 | −2.8511 | 1 | 1 | 1 | 18013 | 0 | −2.8511 |
| PES1 | 4 | 8.22E−07 | 2.47E−06 | 0.00081 | 52 | 4 | −3.747 | 1 | 1 | 1 | 18012 | 0 | −3.747 |
| ACAD9 | 4 | 8.46E−07 | 2.47E−06 | 0.00081 | 53 | 4 | −1.5554 | 1 | 1 | 1 | 18011 | 0 | −1.5554 |
| MTPAP | 4 | 8.53E−07 | 2.47E−06 | 0.00081 | 54 | 4 | −2.9187 | 1 | 1 | 1 | 18010 | 0 | −2.9187 |
| RPP21 | 4 | 8.68E−07 | 2.47E−06 | 0.00081 | 55 | 4 | −2.1418 | 1 | 1 | 1 | 18009 | 0 | −2.1418 |
| CINP | 4 | 9.03E−07 | 3.02E−06 | 0.000923 | 56 | 4 | −1.4883 | 0.98634 | 0.9864 | 1 | 16606 | 0 | −1.4883 |
| DTYMK | 4 | 9.10E−07 | 3.02E−06 | 0.000923 | 57 | 4 | −1.5122 | 1 | 1 | 1 | 18008 | 0 | −1.5122 |
| HSCB | 4 | 9.51E−07 | 3.02E−06 | 0.000923 | 58 | 4 | −1.9282 | 1 | 1 | 1 | 18007 | 0 | −1.9282 |
| IBA57 | 4 | 9.54E−07 | 3.02E−06 | 0.000923 | 59 | 4 | −1.8134 | 1 | 1 | 1 | 18006 | 0 | −1.8134 |
| POLG2 | 4 | 1.09E−06 | 3.56E−06 | 0.001055 | 60 | 4 | −3.3534 | 1 | 1 | 1 | 17996 | 0 | −3.3534 |
| WDR25 | 4 | 1.16E−06 | 3.56E−06 | 0.001055 | 61 | 4 | −1.6049 | 0.9998 | 0.99981 | 1 | 17682 | 0 | −1.6049 |
| TBCB | 4 | 1.36E−06 | 4.66E−06 | 0.001153 | 62 | 4 | −3.6467 | 1 | 1 | 1 | 18005 | 0 | −3.6467 |
| POLR3H | 4 | 1.37E−06 | 4.66E−06 | 0.001153 | 63 | 4 | −2.6949 | 1 | 1 | 1 | 18004 | 0 | −2.6949 |
| YBEY | 4 | 1.39E−06 | 4.66E−06 | 0.001153 | 64 | 4 | −1.368 | 1 | 1 | 1 | 18003 | 0 | −1.368 |
| NDUFAF1 | 4 | 1.42E−06 | 4.66E−06 | 0.001153 | 65 | 4 | −3.1234 | 0.99358 | 0.99369 | 1 | 16963 | 0 | −3.1234 |
| MAT2A | 4 | 1.43E−06 | 4.66E−06 | 0.001153 | 66 | 4 | −1.8168 | 1 | 1 | 1 | 18002 | 0 | −1.8168 |
| EMC1 | 4 | 1.44E−06 | 4.66E−06 | 0.001153 | 67 | 4 | −2.0027 | 1 | 1 | 1 | 18001 | 0 | −2.0027 |
| MTIF2 | 4 | 1.45E−06 | 4.66E−06 | 0.001153 | 68 | 4 | −1.6455 | 1 | 1 | 1 | 18000 | 0 | −1.6455 |
| TOMM40 | 4 | 1.46E−06 | 4.66E−06 | 0.001153 | 69 | 4 | −2.0022 | 1 | 1 | 1 | 17999 | 0 | −2.0022 |
| VARS2 | 4 | 1.48E−06 | 4.66E−06 | 0.001153 | 70 | 4 | −2.3855 | 1 | 1 | 1 | 17998 | 0 | −2.3855 |
| NDNL2 | 4 | 1.51E−06 | 4.66E−06 | 0.001153 | 71 | 4 | −2.0581 | 0.99997 | 0.99997 | 1 | 17852 | 0 | −2.0581 |
| TRIT1 | 4 | 1.58E−06 | 4.66E−06 | 0.001153 | 72 | 4 | −2.2399 | 1 | 1 | 1 | 17997 | 0 | −2.2399 |
| TELO2 | 4 | 1.68E−06 | 4.66E−06 | 0.001153 | 73 | 4 | −1.7507 | 1 | 1 | 1 | 17995 | 0 | −1.7507 |
| ARMC7 | 4 | 1.73E−06 | 5.21E−06 | 0.001161 | 74 | 4 | −1.8631 | 1 | 1 | 1 | 17994 | 0 | −1.8631 |
| DPAGT1 | 4 | 1.74E−06 | 5.21E−06 | 0.001161 | 75 | 4 | −2.1373 | 1 | 1 | 1 | 17993 | 0 | −2.1373 |
| TFB1M | 4 | 1.75E−06 | 5.21E−06 | 0.001161 | 76 | 3 | −2.1553 | 0.76258 | 0.80749 | 1 | 12796 | 1 | −2.1553 |
| MTG1 | 4 | 1.79E−06 | 5.21E−06 | 0.001161 | 77 | 4 | −1.7081 | 0.99998 | 0.99998 | 1 | 17885 | 0 | −1.7081 |
| CPSF1 | 4 | 1.90E−06 | 5.21E−06 | 0.001161 | 78 | 4 | −2.0648 | 1 | 1 | 1 | 17992 | 0 | −2.0648 |
| PTPMT1 | 4 | 2.12E−06 | 5.21E−06 | 0.001161 | 79 | 4 | −2.746 | 1 | 1 | 1 | 17972 | 0 | −2.746 |
| FARS2 | 4 | 2.18E−06 | 5.211−06 | 0.001161 | 80 | 4 | −2.9016 | 1 | 1 | 1 | 17991 | 0 | −2.9016 |
| MRPS6 | 4 | 2.19E−06 | 5.21E−06 | 0.001161 | 81 | 4 | −1.9805 | 0.99999 | 0.99999 | 1 | 17949 | 0 | −1.9805 |
| ALG11 | 4 | 2.21E−06 | 5.76E−06 | 0.001253 | 82 | 4 | −1.3079 | 1 | 1 | 1 | 17990 | 0 | −1.3079 |
| AP2S1 | 4 | 2.37E−06 | 5.76E−06 | 0.001253 | 83 | 4 | −1.8524 | 1 | 1 | 1 | 17989 | 0 | −1.8524 |
| NOC4L | 4 | 2.56E−06 | 6.86E−06 | 0.001473 | 84 | 4 | −1.6174 | 1 | 1 | 1 | 17988 | 0 | −1.6174 |
| SARS2 | 4 | 2.84E−06 | 9.60E−06 | 0.002038 | 85 | 4 | −1.3453 | 1 | 1 | 1 | 17987 | 0 | −1.3453 |
| POLR3K | 4 | 2.94E−06 | 1.01E−05 | 0.00213 | 86 | 4 | −2.1371 | 1 | 1 | 1 | 17986 | 0 | −2.1371 |
| GGPS1 | 4 | 3.11E−06 | 1.07E−05 | 0.002145 | 87 | 4 | −1.5641 | 1 | 1 | 1 | 17985 | 0 | −1.5641 |
| CDIPT | 4 | 3.14E−06 | 1.07E−05 | 0.002145 | 88 | 4 | −2.4922 | 1 | 1 | 1 | 17984 | 0 | −2.4922 |
| TOMM20 | 4 | 3.15E−06 | 1.07E−05 | 0.002145 | 89 | 4 | −1.504 | 0.99997 | 0.99997 | 1 | 17860 | 0 | −1.504 |
| RNASEH1 | 4 | 3.17E−06 | 1.071−05 | 0.002145 | 90 | 4 | −2.2212 | 0.99994 | 0.99995 | 1 | 17808 | 0 | −2.2212 |
| WDR7 | 4 | 3.33E−06 | 1.121−05 | 0.002182 | 91 | 4 | −1.8314 | 1 | 1 | 1 | 17982 | 0 | −1.8314 |
| TXNL4B | 4 | 3.50E−06 | 1.12E−05 | 0.002182 | 92 | 4 | −1.86 | 1 | 1 | 1 | 17981 | 0 | −1.86 |
| ASUN | 4 | 3.56E−06 | 1.12E−05 | 0.002182 | 93 | 4 | −1.7609 | 1 | 1 | 1 | 17980 | 0 | −1.7609 |
| COQ4 | 4 | 3.63E−06 | 1.18E−05 | 0.002217 | 94 | 4 | −1.9958 | 1 | 1 | 1 | 17979 | 0 | −1.9958 |
| C9orf114 | 4 | 3.64E−06 | 1.18E−05 | 0.002217 | 95 | 4 | −2.1391 | 1 | 1 | 1 | 17978 | 0 | −2.1391 |
| UTP3 | 4 | 3.83E−06 | 1.18E−05 | 0.002217 | 96 | 4 | −1.5292 | 1 | 1 | 1 | 17977 | 0 | −1.5292 |
| DNAJC17 | 4 | 3.86E−06 | 1.23E−05 | 0.00225 | 97 | 4 | −2.0344 | 1 | 1 | 1 | 17976 | 0 | −2.0344 |
| ARPC4 | 4 | 3.90E−06 | 1.23E−05 | 0.00225 | 98 | 4 | −1.2976 | 1 | 1 | 1 | 17975 | 0 | −1.2976 |
| C10orf2 | 4 | 4.08E−06 | 1.23E−05 | 0.00225 | 99 | 4 | −3.1909 | 1 | 1 | 1 | 17974 | 0 | −3.1909 |
| WARS2 | 4 | 4.11E−06 | 1.29E−05 | 0.002281 | 100 | 4 | −1.8989 | 1 | 1 | 1 | 17973 | 0 | −1.8989 |
| TFAM | 4 | 4.17E−06 | 1.29E−05 | 0.002281 | 101 | 4 | −1.7056 | 1 | 1 | 1 | 17971 | 0 | −1.7056 |
| DAP3 | 4 | 4.28E−06 | 1.29E−05 | 0.002281 | 102 | 4 | −2.7925 | 1 | 1 | 1 | 17970 | 0 | −2.7925 |
| ARFRP1 | 4 | 4.34E−06 | 1.34E−05 | 0.002332 | 103 | 4 | −1.3536 | 1 | 1 | 1 | 17969 | 0 | −1.3536 |
| NDUFA1 | 4 | 4.37E−06 | 1.34E−05 | 0.002332 | 104 | 4 | −1.1669 | 1 | 1 | 1 | 17968 | 0 | −1.1669 |
| TOMM22 | 4 | 4.52E−06 | 1.51E−05 | 0.002593 | 105 | 1 | −2.1607 | 1 | 1 | 1 | 17967 | 0 | −2.1607 |
| COX17 | 4 | 4.59E−06 | 1.56E−05 | 0.002608 | 106 | 4 | −1.4425 | 1 | 1 | 1 | 17966 | 0 | −1.4425 |
| PNPT1 | 4 | 4.62E−06 | 1.56E−05 | 0.002608 | 107 | 4 | −3.0345 | 1 | 1 | 1 | 17965 | 0 | −3.0345 |
| NARS | 4 | 4.65E−06 | 1.62E−05 | 0.002608 | 108 | 4 | −3.4438 | 1 | 1 | 1 | 17964 | 0 | −3.4438 |
| PPIH | 4 | 4.65E−06 | 1.62E−05 | 0.002608 | 109 | 4 | −1.0948 | 1 | 1 | 1 | 17963 | 0 | −1.0948 |
| ABT1 | 4 | 4.67E−06 | 1.62E−05 | 0.002608 | 110 | 4 | −1.4172 | 1 | 1 | 1 | 17962 | 0 | −1.4172 |
| NELFB | 4 | 4.68E−06 | 1.62E−05 | 0.002608 | 111 | 4 | −2.9919 | 1 | 1 | 1 | 17961 | 0 | −2.9919 |
| AHCYL1 | 4 | 4.69E−06 | 1.62E−05 | 0.002608 | 112 | 4 | −1.2955 | 1 | 1 | 1 | 17960 | 0 | −1.2955 |
| RBBP5 | 4 | 4.74E−06 | 1.67E−05 | 0.002649 | 113 | 4 | −1.12 | 1 | 1 | 1 | 17959 | 0 | −1.12 |
| IPO13 | 4 | 4.77E−06 | 1.67E−05 | 0.002649 | 114 | 4 | −1.9726 | 1 | 1 | 1 | 17958 | 0 | −1.9726 |
| NUDC | 4 | 5.10E−06 | 1.73E−05 | 0.002689 | 115 | 4 | −2.5874 | 0.99999 | 1 | 1 | 17957 | 0 | −2.5874 |
| RBM17 | 4 | 5.18E−06 | 1.73E−05 | 0.002689 | 116 | 4 | −1.5192 | 0.99999 | 1 | 1 | 17956 | 0 | −1.5192 |
| NOL9 | 4 | 5.50E−06 | 1.78E−05 | 0.002727 | 117 | 4 | −1.9949 | 0.99999 | 1 | 1 | 17955 | 0 | −1.9949 |
| MED8 | 4 | 5.53E−06 | 1.78E−05 | 0.002727 | 118 | 4 | −1.2661 | 0.99999 | 1 | 1 | 17954 | 0 | −1.2661 |
| PARS2 | 4 | 5.68E−06 | 1.84E−05 | 0.002764 | 119 | 4 | −3.0321 | 0.99979 | 0.99979 | 1 | 17675 | 0 | −3.0321 |
| GEMIN7 | 4 | 5.68E−06 | 1.84E−05 | 0.002764 | 120 | 4 | −2.0035 | 0.99999 | 1 | 1 | 17953 | 0 | −2.0035 |
| RTEL1 | 4 | 5.82E−06 | 1.89E−05 | 0.002823 | 121 | 4 | −1.7929 | 0.99999 | 1 | 1 | 17952 | 0 | −1.7929 |
| RAD1 | 4 | 6.24E−06 | 2.00E−05 | 0.002846 | 122 | 4 | −1.5974 | 0.99999 | 0.99999 | 1 | 17951 | 0 | −1.5974 |
| MTOR | 4 | 6.24E−06 | 2.00E−05 | 0.002846 | 123 | 4 | −1.7148 | 0.99999 | 0.99999 | 1 | 17950 | 0 | −1.7148 |
| IMP3 | 4 | 6.27E−06 | 2.00E−05 | 0.002846 | 124 | 4 | −3.2505 | 0.99999 | 0.99999 | 1 | 17948 | 0 | −3.2505 |
| SEC63 | 4 | 6.27E−06 | 2.00E−05 | 0.002846 | 125 | 4 | −1.724 | 0.99989 | 0.99989 | 1 | 17741 | 0 | −1.724 |
| C14orf80 | 4 | 6.48E−06 | 2.00E−05 | 0.002846 | 126 | 4 | −2.1121 | 0.97999 | 0.98009 | 1 | 16357 | 0 | −2.1121 |
| FNTB | 4 | 6.51E−06 | 2.00E−05 | 0.002846 | 127 | 4 | −1.9943 | 0.99999 | 0.99999 | 1 | 17947 | 0 | −1.9943 |
| WDR73 | 4 | 6.78E−06 | 2.11E−05 | 0.002932 | 128 | 4 | −2.5884 | 0.99993 | 0.99994 | 1 | 17793 | 0 | −2.5884 |
| XRCC2 | 4 | 6.91E−06 | 2.11E−05 | 0.002932 | 129 | 3 | −1.3817 | 0.90193 | 0.90162 | 1 | 14490 | 1 | −1.3817 |
| TIMM22 | 4 | 6.91E−06 | 2.11E−05 | 0.002932 | 130 | 4 | −1.2241 | 0.99999 | 0.99999 | 1 | 17946 | 0 | −1.2241 |
| GFM1 | 4 | 7.03E−06 | 2.17E−05 | 0.002985 | 131 | 4 | −1.4188 | 0.99999 | 0.99999 | 1 | 17945 | 0 | −1.4188 |
| TAF1C | 4 | 7.35E−06 | 2.33E−05 | 0.003099 | 132 | 4 | −1.6303 | 0.99999 | 0.99999 | 1 | 17944 | 0 | −1.6303 |
| MRPL15 | 4 | 7.51E−06 | 2.33E−05 | 0.003099 | 133 | 4 | −2.6255 | 0.99999 | 0.99999 | 1 | 17943 | 0 | −2.6255 |
| ELP5 | 4 | 7.55E−06 | 2.33E−05 | 0.003099 | 134 | 4 | −2.2212 | 0.99999 | 0.99999 | 1 | 17942 | 0 | −2.2212 |
| UBIAD1 | 4 | 7.60E−06 | 2.39E−05 | 0.003099 | 135 | 4 | −1.2292 | 0.99999 | 0.99999 | 1 | 17941 | 0 | −1.2292 |
| EXOSC5 | 4 | 7.65E−06 | 2.39E−05 | 0.003099 | 136 | 4 | −2.4484 | 0.99999 | 0.99999 | 1 | 17940 | 0 | −2.4484 |
| FDXR | 4 | 7.66E−06 | 2.39E−05 | 0.003099 | 137 | 3 | −2.0419 | 0.92211 | 0.92192 | 1 | 14866 | 0 | −2.0419 |
| C21orf59 | 4 | 7.67E−06 | 2.39E−05 | 0.003099 | 138 | 4 | −2.1797 | 0.99999 | 0.99999 | 1 | 17939 | 0 | −2.1797 |
| LARS2 | 4 | 7.74E−06 | 2.39E−05 | 0.003099 | 139 | 4 | −2.4556 | 0.99908 | 0.99907 | 1 | 17475 | 0 | −2.4556 |
| TCEB2 | 4 | 7.85E−06 | 2.55E−05 | 0.003289 | 140 | 4 | −2.738 | 0.99999 | 0.99999 | 1 | 17938 | 0 | −2.738 |
| RPP14 | 4 | 8.01E−06 | 2.61E−05 | 0.003312 | 141 | 4 | −1.3047 | 0.99999 | 0.99999 | 1 | 17937 | 0 | −1.3047 |
| TAF13 | 4 | 8.06E−06 | 2.61E−05 | 0.003312 | 142 | 4 | −1.4807 | 0.99999 | 0.99999 | 1 | 17936 | 0 | −1.4807 |
| ATP6V1E1 | 4 | 8.10E−06 | 2.66E−05 | 0.003358 | 143 | 4 | −1.4127 | 0.99999 | 0.99999 | 1 | 17935 | 0 | −1.4127 |
| MARS2 | 4 | 8.28E−06 | 2.77E−05 | 0.003448 | 144 | 4 | −2.2985 | 0.99999 | 0.99999 | 1 | 17934 | 0 | −2.2985 |
| ZFYVE20 | 4 | 8.40E−06 | 2.77E−05 | 0.003448 | 145 | 4 | −1.6078 | 0.99999 | 0.99999 | 1 | 17933 | 0 | −1.6078 |
| MRPS34 | 4 | 8.65E−06 | 2.82E−05 | 0.003492 | 146 | 4 | −2.7098 | 0.99999 | 0.99999 | 1 | 17932 | 0 | −2.7098 |
| DDX59 | 3 | 8.76E−06 | 3.37E−05 | 0.003954 | 147 | 3 | −2.0031 | 0.99999 | 0.99999 | 1 | 17931 | 0 | −2.0031 |
| PFN1 | 4 | 8.84E−06 | 2.99E−05 | 0.003646 | 148 | 4 | −1.8506 | 0.99999 | 0.99999 | 1 | 17930 | 0 | −1.8506 |
| ADAT3 | 4 | 8.86E−06 | 2.99E−05 | 0.003646 | 149 | 3 | −1.8289 | 0.94691 | 0.94681 | 1 | 15407 | 0 | −1.8289 |
| NAE1 | 4 | 8.91E−06 | 3.04E−05 | 0.003688 | 150 | 3 | −2.0648 | 0.9419 | 0.94177 | 1 | 15297 | 0 | −2.0648 |
| TMEM167B | 4 | 9.10E−06 | 3.15E−05 | 0.003786 | 151 | 4 | −1.0646 | 0.99999 | 0.99999 | 1 | 17929 | 0 | −1.0646 |
| NOP16 | 4 | 9.22E−06 | 3.21E−05 | 0.003786 | 152 | 4 | −1.9322 | 0.99999 | 0.99999 | 1 | 17928 | 0 | −1.9322 |
| SKIV2L2 | 4 | 9.24E−06 | 3.21E−05 | 0.003786 | 153 | 4 | −2.025 | 0.99999 | 0.99999 | 1 | 17927 | 0 | −2.025 |
| URB1 | 4 | 9.26E−06 | 3.21E−05 | 0.003786 | 154 | 4 | −2.3783 | 0.99999 | 0.99999 | 1 | 17926 | 0 | −2.3783 |
| ILF3 | 4 | 9.54E−06 | 3.43E−05 | 0.003954 | 155 | 4 | −1.0982 | 0.99999 | 0.99999 | 1 | 17925 | 0 | −1.0982 |
| FOXRED1 | 4 | 9.62E−06 | 3.48E−05 | 0.003954 | 156 | 4 | −2.0379 | 0.99999 | 0.99999 | 1 | 17924 | 0 | −2.0379 |
| TSEN2 | 4 | 1.00E−05 | 3.54E−05 | 0.003954 | 157 | 4 | −1.0891 | 0.99999 | 0.99999 | 1 | 17923 | 0 | −1.0891 |
| ATP5SL | 4 | 1.01E−05 | 3.54E−05 | 0.003954 | 158 | 4 | −1.7334 | 0.99999 | 0.99999 | 1 | 17922 | 0 | −1.7334 |
| MRPL4 | 4 | 1.04E−05 | 3.54E−05 | 0.003954 | 159 | 4 | −1.7837 | 0.99995 | 0.99995 | 1 | 17818 | 0 | −1.7837 |
| TFB2M | 4 | 1.05E−05 | 3.59E−05 | 0.003954 | 160 | 4 | −1.5628 | 0.99997 | 0.99997 | 1 | 17847 | 0 | −1.5628 |
| GEMIN8 | 4 | 1.07E−05 | 3.59E−05 | 0.003954 | 161 | 4 | −1.9213 | 0.99999 | 0.99999 | 1 | 17921 | 0 | −1.9213 |
| CSTF1 | 4 | 1.08E−05 | 3.59E−05 | 0.003954 | 162 | 4 | −1.7619 | 0.99999 | 0.99999 | 1 | 17920 | 0 | −1.7619 |
| MRPS12 | 4 | 1.09E−05 | 3.59E−05 | 0.003954 | 163 | 4 | −1.8817 | 0.99999 | 0.99999 | 1 | 17919 | 0 | −1.8817 |
| MRPL22 | 4 | 1.10E−05 | 3.59E−05 | 0.003954 | 164 | 4 | −1.4575 | 0.99999 | 0.99999 | 1 | 17918 | 0 | −1.4575 |
| CCDC51 | 4 | 1.11E−05 | 3.65E−05 | 0.00399 | 165 | 4 | −1.6641 | 0.99999 | 0.99999 | 1 | 17917 | 0 | −1.6641 |
| GLRX5 | 4 | 1.11E−05 | 3.70E−05 | 0.004026 | 166 | 4 | −2.3673 | 0.99999 | 0.99999 | 1 | 17916 | 0 | −2.3673 |
| WDR5 | 4 | 1.12E−05 | 3.81E−05 | 0.00412 | 167 | 4 | −1.9168 | 0.99999 | 0.99999 | 1 | 17915 | 0 | −1.9168 |
| NDUFA11 | 4 | 1.13E−05 | 3.87E−05 | 0.004155 | 168 | 4 | −1.7229 | 0.99999 | 0.99999 | 1 | 17914 | 0 | −1.7229 |
| NOA1 | 4 | 1.16E−05 | 4.03E−05 | 0.004281 | 169 | 4 | −2.0988 | 0.99999 | 0.99999 | 1 | 17913 | 0 | −2.0988 |
| PPP1R8 | 4 | 1.16E−05 | 4.03E−05 | 0.004281 | 170 | 4 | −1.6419 | 0.99999 | 0.99999 | 1 | 17912 | 0 | −1.6419 |
| BAK1 | 4 | 1.19E−05 | 4.31E−05 | 0.004519 | 171 | 4 | −1.92 | 0.99999 | 0.99999 | 1 | 17911 | 0 | −1.92 |
| FASTKD2 | 4 | 1.19E−05 | 4.31E−05 | 0.004519 | 172 | 4 | −1.2833 | 0.99999 | 0.99999 | 1 | 17910 | 0 | −1.2833 |
| RPUSD4 | 4 | 1.20E−05 | 4.36E−05 | 0.004524 | 173 | 3 | −2.2188 | 0.94748 | 0.94739 | 1 | 15420 | 0 | −2.2188 |
| GRSF1 | 3 | 1.21E−05 | 4.52E−05 | 0.004641 | 174 | 3 | −2.5839 | 0.99999 | 0.99998 | 1 | 17909 | 0 | −2.5839 |
| IKBKAP | 4 | 1.21E−05 | 4.36E−05 | 0.004524 | 175 | 4 | −1.2973 | 0.99994 | 0.99995 | 1 | 17809 | 0 | −1.2973 |
| MAD2L2 | 4 | 1.22E−05 | 4.41E−05 | 0.004554 | 176 | 4 | −2.0418 | 0.99999 | 0.99999 | 1 | 17908 | 0 | −2.0418 |
| CHORDC1 | 4 | 1.24E−05 | 4.58E−05 | 0.004671 | 177 | 4 | −2.9874 | 0.99999 | 0.99999 | 1 | 17907 | 0 | −2.9874 |
| CHTF8 | 4 | 1.26E−05 | 4.63E−05 | 0.0047 | 178 | 4 | −1.0037 | 0.99999 | 0.99999 | 1 | 17906 | 0 | −1.0037 |
| DDX51 | 4 | 1.31E−05 | 4.80E−05 | 0.00484 | 179 | 4 | −1.6642 | 0.99999 | 0.99999 | 1 | 17905 | 0 | −1.6642 |
| AASDHPPT | 4 | 1.33E−05 | 4.85E−05 | 0.004841 | 180 | 4 | −1.309 | 0.99121 | 0.99132 | 1 | 16847 | 0 | −1.309 |
| TRAPPC1 | 4 | 1.33E−05 | 4.85E−05 | 0.004841 | 181 | 4 | −1.9322 | 0.99999 | 0.99999 | 1 | 17904 | 0 | −1.9322 |
| HSD17B10 | 4 | 1.35E−05 | 4.91E−05 | 0.004842 | 182 | 4 | −2.7922 | 0.99992 | 0.99992 | 1 | 17773 | 0 | −2.7922 |
| DCTN6 | 4 | 1.36E−05 | 4.91E−05 | 0.004842 | 183 | 4 | −1.7663 | 0.99999 | 0.99999 | 1 | 17903 | 0 | −1.7663 |
| TYMS | 4 | 1.38E−05 | 5.02E−05 | 0.004924 | 184 | 4 | −1.5324 | 0.99984 | 0.99985 | 1 | 17708 | 0 | −1.5324 |
| PTDSS1 | 4 | 1.42E−05 | 5.07E−05 | 0.004924 | 185 | 4 | −1.4242 | 0.99999 | 0.99999 | 1 | 17902 | 0 | −1.4242 |
| MRPL35 | 4 | 1.43E−05 | 5.07E−05 | 0.004924 | 186 | 4 | −1.8373 | 0.99999 | 0.99999 | 1 | 17901 | 0 | −1.8373 |
| CXXC1 | 4 | 1.46E−05 | 5.24E−05 | 0.005056 | 187 | 4 | −1.0847 | 0.99999 | 0.99998 | 1 | 17900 | 0 | −1.0847 |
| MRPS28 | 4 | 1.48E−05 | 5.29E−05 | 0.005082 | 188 | 4 | −1.1431 | 0.99999 | 0.99998 | 1 | 17899 | 0 | −1.1431 |
| TANGO6 | 4 | 1.49E−05 | 5.35E−05 | 0.005108 | 189 | 3 | −1.6686 | 0.056165 | 0.14794 | 0.752932 | 3501 | 1 | −1.6686 |
| MRPL34 | 4 | 1.57E−05 | 5.51E−05 | 0.005237 | 190 | 4 | −3.05 | 0.99998 | 0.99998 | 1 | 17898 | 0 | −3.05 |
| GAPDH | 4 | 1.58E−05 | 5.57E−05 | 0.005262 | 191 | 4 | −1.7888 | 0.99998 | 0.99998 | 1 | 17876 | 0 | −1.7888 |
| NSMCE2 | 4 | 1.64E−05 | 6.01E−05 | 0.005647 | 192 | 4 | −1.0234 | 0.99998 | 0.99998 | 1 | 17896 | 0 | −1.0234 |
| EXOSC3 | 4 | 1.66E−05 | 6.17E−05 | 0.005771 | 193 | 4 | −2.3107 | 0.99998 | 0.99998 | 1 | 17895 | 0 | −2.3107 |
| TSFM | 4 | 1.67E−05 | 6.28E−05 | 0.00582 | 194 | 4 | −2.0647 | 0.99994 | 0.99995 | 1 | 17815 | 0 | −2.0647 |
| TBCA | 4 | 1.69E−05 | 6.33E−05 | 0.00582 | 195 | 4 | −2.8119 | 0.99854 | 0.99855 | 1 | 17376 | 0 | −2.8119 |
| AIFM1 | 4 | 1.70E−05 | 6.39E−05 | 0.00582 | 196 | 4 | −1.8517 | 0.99998 | 0.99998 | 1 | 17894 | 0 | −1.8517 |
| FAM96B | 4 | 1.70E−05 | 6.39E−05 | 0.00582 | 197 | 4 | −2.9086 | 0.99948 | 0.9995 | 1 | 17562 | 0 | −2.9086 |
| CDC123 | 4 | 1.71E−05 | 6.39E−05 | 0.00582 | 198 | 4 | −1.8783 | 0.99998 | 0.99998 | 1 | 17893 | 0 | −1.8783 |
| CARS2 | 4 | 1.72E−05 | 6.44E−05 | 0.00582 | 199 | 4 | −2.8723 | 0.99998 | 0.99998 | 1 | 17892 | 0 | −2.8723 |
| TEN1 | 4 | 1.75E−05 | 6.50E−05 | 0.00582 | 200 | 4 | −1.3103 | 0.99754 | 0.99757 | 1 | 17264 | 0 | −1.3103 |
| PPIL2 | 4 | 1.77E−05 | 6.55E−05 | 0.00582 | 201 | 4 | −1.6785 | 0.99998 | 0.99998 | 1 | 17891 | 0 | −1.6785 |
| ORC5 | 4 | 1.79E−05 | 6.61E−05 | 0.00582 | 202 | 4 | −1.2293 | 0.99998 | 0.99998 | 1 | 17890 | 0 | −1.2293 |
| DOHH | 4 | 1.79E−05 | 6.61E−05 | 0.00582 | 203 | 4 | −1.2578 | 0.99978 | 0.99979 | 1 | 17672 | 0 | −1.2578 |
| TDP2 | 4 | 1.80E−05 | 6.61E−05 | 0.00582 | 204 | 4 | −1.2794 | 0.99998 | 0.99998 | 1 | 17889 | 0 | −1.2794 |
| TRMT61A | 4 | 1.80E−05 | 6.61E−05 | 0.00582 | 205 | 3 | −1.7109 | 0.14266 | 0.29304 | 0.91613 | 5710 | 1 | −1.7109 |
| ENO1 | 4 | 1.82E−05 | 6.72E−05 | 0.005859 | 206 | 4 | −1.7021 | 0.99998 | 0.99998 | 1 | 17867 | 0 | −1.7021 |
| WRB | 4 | 1.83E−05 | 6.72E−05 | 0.005859 | 207 | 4 | −1.1532 | 0.99998 | 0.99998 | 1 | 17888 | 0 | −1.1532 |
| RTN4IP1 | 4 | 1.84E−05 | 6.83E−05 | 0.005926 | 208 | 4 | −1.537 | 0.99998 | 0.99998 | 1 | 17886 | 0 | −1.537 |
| TUBE1 | 4 | 1.91E−05 | 7.16E−05 | 0.006182 | 209 | 4 | −1.9635 | 0.99998 | 0.99998 | 1 | 17887 | 0 | −1.9635 |
| HIGD2A | 4 | 1.99E−05 | 7.38E−05 | 0.006341 | 210 | 4 | −1.7593 | 0.99972 | 0.99973 | 1 | 17648 | 0 | −1.7593 |
| MIPEP | 4 | 2.04E−05 | 7.54E−05 | 0.006438 | 211 | 4 | −1.8051 | 0.99998 | 0.99998 | 1 | 17884 | 0 | −1.8051 |
| POLR1E | 4 | 2.06E−05 | 7.60E−05 | 0.006438 | 212 | 4 | −1.8653 | 0.99998 | 0.99998 | 1 | 17883 | 0 | −1.8653 |
| POP5 | 4 | 2.07E−05 | 7.60E−05 | 0.006438 | 213 | 4 | −2.2345 | 0.99998 | 0.99998 | 1 | 17882 | 0 | −2.2345 |
| ILF2 | 4 | 2.12E−05 | 7.82E−05 | 0.006562 | 214 | 4 | −2.3065 | 0.99998 | 0.99998 | 1 | 17881 | 0 | −2.3065 |
| SNUPN | 4 | 2.12E−05 | 7.82E−05 | 0.006562 | 215 | 4 | −1.7985 | 0.99996 | 0.99996 | 1 | 17832 | 0 | −1.7985 |
| ARMC5 | 4 | 2.15E−05 | 7.87E−05 | 0.006578 | 216 | 4 | −1.1964 | 0.99998 | 0.99998 | 1 | 17880 | 0 | −1.1964 |
| MARS | 4 | 2.20E−05 | 8.09E−05 | 0.006714 | 217 | 4 | −1.7995 | 0.99998 | 0.99998 | 1 | 17879 | 0 | −1.7995 |
| NSMCE1 | 4 | 2.21E−05 | 8.14E−05 | 0.006714 | 218 | 4 | −2.0198 | 0.99998 | 0.99998 | 1 | 17878 | 0 | −2.0198 |
| DENND2D | 4 | 2.21E−05 | 8.14E−05 | 0.006714 | 219 | 4 | −0.98471 | 0.99998 | 0.99998 | 1 | 17877 | 0 | −0.98471 |
| XRN2 | 4 | 2.23E−05 | 8.25E−05 | 0.006743 | 220 | 4 | −1.5979 | 0.99998 | 0.99998 | 1 | 17875 | 0 | −1.5979 |
| MRPL12 | 4 | 2.25E−05 | 8.25E−05 | 0.006743 | 221 | 4 | −1.5774 | 0.99998 | 0.99998 | 1 | 17874 | 0 | −1.5774 |
| EXOSC7 | 4 | 2.26E−05 | 8.31E−05 | 0.006757 | 222 | 4 | −2.9181 | 0.97432 | 0.97427 | 1 | 16172 | 0 | −2.9181 |
| SEC61B | 4 | 2.27E−05 | 8.42E−05 | 0.006815 | 223 | 4 | −1.2871 | 0.99998 | 0.99998 | 1 | 17873 | 0 | −1.2871 |
| GTF2H1 | 4 | 2.32E−05 | 8.75E−05 | 0.006983 | 224 | 4 | −0.94736 | 0.99998 | 0.99998 | 1 | 17872 | 0 | −0.94736 |
| GCSH | 4 | 2.33E−05 | 8.80E−05 | 0.006983 | 225 | 4 | −1.1899 | 0.99998 | 0.99998 | 1 | 17871 | 0 | −1.1899 |
| METTL3 | 4 | 2.34E−05 | 8.80E−05 | 0.006983 | 226 | 4 | −1.4872 | 0.99996 | 0.99996 | 1 | 17846 | 0 | −1.4872 |
| GTF2A2 | 4 | 2.34E−05 | 8.80E−05 | 0.006983 | 227 | 4 | −1.8974 | 0.99998 | 0.99998 | 1 | 17869 | 0 | −1.8974 |
| BCCIP | 4 | 2.37E−05 | 8.86E−05 | 0.006983 | 228 | 4 | −1.5031 | 0.99896 | 0.99895 | 1 | 17448 | 0 | −1.5031 |
| EMC6 | 4 | 2.37E−05 | 8.86E−05 | 0.006983 | 229 | 3 | −1.2335 | 0.2431 | 0.43801 | 1 | 7787 | 1 | −1.2335 |
| WDR3 | 4 | 2.38E−05 | 8.91E−05 | 0.006995 | 230 | 4 | −1.7072 | 0.99998 | 0.99998 | 1 | 17868 | 0 | −1.7072 |
| NVL | 4 | 2.50E−05 | 9.35E−05 | 0.007276 | 231 | 4 | −1.7998 | 0.99997 | 0.99997 | 1 | 17866 | 0 | −1.7998 |
| EFTUD1 | 4 | 2.50E−05 | 9.35E−05 | 0.007276 | 232 | 4 | −2.1163 | 0.99997 | 0.99997 | 1 | 17865 | 0 | −2.1163 |
| MAK16 | 4 | 2.55E−05 | 9.41E−05 | 0.007288 | 233 | 4 | −1.6439 | 0.99997 | 0.99997 | 1 | 17864 | 0 | −1.6439 |
| PDSS1 | 4 | 2.80E−05 | 0.00010283 | 0.007854 | 234 | 4 | −1.6634 | 0.99997 | 0.99997 | 1 | 17863 | 0 | −1.6634 |
| ZC3H18 | 4 | 2.80E−05 | 0.00010283 | 0.007854 | 235 | 4 | −1.3517 | 0.99997 | 0.99997 | 1 | 17862 | 0 | −1.3517 |
| ELAC2 | 4 | 2.81E−05 | 0.00010283 | 0.007854 | 236 | 4 | −1.3307 | 0.99965 | 0.99967 | 1 | 17626 | 0 | −1.3307 |
| SLC7A5 | 4 | 2.81E−05 | 0.00010311 | 0.007854 | 237 | 4 | −1.3497 | 0.99997 | 0.99997 | 1 | 17861 | 0 | −1.3497 |
| CDS2 | 4 | 2.83E−05 | 0.00010393 | 0.007883 | 238 | 4 | −1.1248 | 0.99997 | 0.99997 | 1 | 17859 | 0 | −1.1248 |
| COX15 | 4 | 2.84E−05 | 0.00010503 | 0.0079 | 239 | 4 | −1.8315 | 0.99985 | 0.99986 | 1 | 17713 | 0 | −1.8315 |
| MRPL39 | 4 | 2.85E−05 | 0.00010557 | 0.007908 | 240 | 3 | −2.2826 | 0.88094 | 0.88257 | 1 | 14142 | 1 | −2.2826 |
| ATP5F1 | 4 | 2.90E−05 | 0.00010722 | 0.007966 | 241 | 4 | −1.5274 | 0.99997 | 0.99997 | 1 | 17858 | 0 | −1.5274 |
| WDR77 | 4 | 2.91E−05 | 0.00010722 | 0.007966 | 242 | 4 | −3.2406 | 0.99993 | 0.99994 | 1 | 17794 | 0 | −3.2406 |
| ZNF407 | 4 | 2.95E−05 | 0.00010777 | 0.007974 | 243 | 4 | −1.6068 | 0.99997 | 0.99997 | 1 | 17857 | 0 | −1.6068 |
| CHCHD4 | 4 | 3.06E−05 | 0.00011051 | 0.008143 | 244 | 4 | −1.5691 | 0.99997 | 0.99997 | 1 | 17856 | 0 | −1.5691 |
| RBM14 | 4 | 3.15E−05 | 0.00011325 | 0.008311 | 245 | 2 | −1.3152 | 0.95136 | 0.95126 | 1 | 15520 | 0 | −1.3152 |
| NDUFAF6 | 4 | 3.16E−05 | 0.00011435 | 0.008358 | 246 | 3 | −1.2557 | 0.86563 | 0.87043 | 1 | 13910 | 1 | −1.2557 |
| NOB1 | 4 | 3.18E−05 | 0.0001149 | 0.008364 | 247 | 4 | −1.4095 | 0.99997 | 0.99997 | 1 | 17855 | 0 | −1.4095 |
| GMPPB | 3 | 3.20E−05 | 0.00010503 | 0.0079 | 248 | 3 | −2.0948 | 0.99997 | 0.99997 | 1 | 17854 | 0 | −2.0948 |
| ERAL1 | 4 | 3.24E−05 | 0.00011709 | 0.008489 | 249 | 4 | −2.4238 | 0.99703 | 0.99707 | 1 | 17201 | 0 | −2.4238 |
| MRPL47 | 4 | 3.26E−05 | 0.00011764 | 0.008495 | 250 | 4 | −1.767 | 0.99997 | 0.99997 | 1 | 17853 | 0 | −1.767 |
| CMPK1 | 4 | 3.34E−05 | 0.00012093 | 0.008673 | 251 | 3 | −2.1728 | 0.76201 | 0.80718 | 1 | 12789 | 1 | −2.1728 |
| CIRH1A | 4 | 3.37E−05 | 0.00012148 | 0.008673 | 252 | 4 | −1.9514 | 0.99996 | 0.99996 | 1 | 17835 | 0 | −1.9514 |
| BRD2 | 4 | 3.39E−05 | 0.00012203 | 0.008673 | 253 | 4 | −1.098 | 0.99997 | 0.99997 | 1 | 17851 | 0 | −1.098 |
| SRP9 | 4 | 3.39E−05 | 0.00012203 | 0.008673 | 254 | 4 | −1.6691 | 0.99997 | 0.99997 | 1 | 17848 | 0 | −1.6691 |
| GTF3C3 | 4 | 3.40E−05 | 0.00012312 | 0.008683 | 255 | 4 | −1.1663 | 0.99997 | 0.99997 | 1 | 17850 | 0 | −1.1663 |
| ATP2A2 | 4 | 3.41E−05 | 0.00012312 | 0.008683 | 256 | 4 | −3.2446 | 0.99997 | 0.99997 | 1 | 17849 | 0 | −3.2446 |
| RPP38 | 4 | 3.53E−05 | 0.00013025 | 0.00915 | 257 | 4 | −1.4766 | 0.99943 | 0.99945 | 1 | 17546 | 0 | −1.4766 |
| NUBPL | 4 | 3.61E−05 | 0.0001319 | 0.009229 | 258 | 4 | −1.7559 | 0.98376 | 0.98381 | 1 | 16490 | 0 | −1.7559 |
| MRPL44 | 4 | 3.70E−05 | 0.00013574 | 0.009461 | 259 | 4 | −1.4535 | 0.99996 | 0.99996 | 1 | 17845 | 0 | −1.4535 |
| E4F1 | 4 | 3.72E−05 | 0.00013629 | 0.009463 | 260 | 4 | −1.3123 | 0.99996 | 0.99996 | 1 | 17844 | 0 | −1.3123 |
| DCLRE1B | 4 | 3.82E−05 | 0.00014287 | 0.009863 | 261 | 4 | −1.4544 | 0.99996 | 0.99996 | 1 | 17843 | 0 | −1.4544 |
| SSU72 | 4 | 3.85E−05 | 0.00014342 | 0.009863 | 262 | 4 | −2.218 | 0.99996 | 0.99996 | 1 | 17842 | 0 | −2.218 |
| THOC7 | 4 | 3.87E−05 | 0.00014369 | 0.009863 | 263 | 4 | −1.5144 | 0.99994 | 0.99994 | 1 | 17799 | 0 | −1.5144 |
| TIMM50 | 4 | 3.91E−05 | 0.00014561 | 0.00992 | 264 | 3 | −2.0279 | 0.47799 | 0.63471 | 1 | 10275 | 1 | −2.0279 |
| DNM1L | 4 | 3.95E−05 | 0.00014616 | 0.00992 | 265 | 4 | −1.2225 | 0.99996 | 0.99996 | 1 | 17841 | 0 | −1.2225 |
| C19orf52 | 4 | 3.96E−05 | 0.00014616 | 0.00992 | 266 | 4 | −1.6058 | 0.99996 | 0.99996 | 1 | 17840 | 0 | −1.6058 |
| SPATA5L1 | 4 | 4.08E−05 | 0.00015219 | 0.010289 | 267 | 4 | −2.4976 | 0.9624 | 0.9623 | 1 | 15804 | 0 | −2.4976 |
| LONP1 | 4 | 4.10E−05 | 0.00015274 | 0.010289 | 268 | 4 | −2.0743 | 0.99996 | 0.99996 | 1 | 17839 | 0 | −2.0743 |
| PTBP1 | 4 | 4.14E−05 | 0.00015384 | 0.010324 | 269 | 4 | −1.6598 | 0.99996 | 0.99996 | 1 | 17838 | 0 | −1.6598 |
| CCS | 4 | 4.19E−05 | 0.00015658 | 0.010463 | 270 | 3 | −1.8932 | 0.95196 | 0.95184 | 1 | 15534 | 0 | −1.8932 |
| RIOK2 | 4 | 4.20E−05 | 0.00015713 | 0.010463 | 271 | 4 | −1.3001 | 0.99996 | 0.99996 | 1 | 17837 | 0 | −1.3001 |
| QRSL1 | 4 | 4.22E−05 | 0.00015768 | 0.010463 | 272 | 4 | −2.2811 | 0.99936 | 0.99938 | 1 | 17524 | 0 | −2.2811 |
| NSUN4 | 4 | 4.23E−05 | 0.00015822 | 0.010463 | 273 | 4 | −1.662 | 0.99506 | 0.99515 | 1 | 17065 | 0 | −1.662 |
| ATP5B | 4 | 4.32E−05 | 0.00016097 | 0.010606 | 274 | 4 | −1.539 | 0.99996 | 0.99996 | 1 | 17836 | 0 | −1.539 |
| UBA2 | 4 | 4.38E−05 | 0.00016289 | 0.010659 | 275 | 4 | −1.3416 | 0.99996 | 0.99996 | 1 | 17834 | 0 | −1.3416 |
| TSEN54 | 4 | 4.39E−05 | 0.00016316 | 0.010659 | 276 | 4 | −2.7322 | 0.99996 | 0.99996 | 1 | 17833 | 0 | −2.7322 |
| CTC1 | 4 | 4.43E−05 | 0.00016481 | 0.010659 | 277 | 4 | −1.5455 | 0.99452 | 0.99462 | 1 | 17024 | 0 | −1.5455 |
| VPS16 | 4 | 4.44E−05 | 0.00016481 | 0.010659 | 278 | 4 | −1.5038 | 0.99971 | 0.99972 | 1 | 17647 | 0 | −1.5038 |
| MRPL13 | 4 | 4.48E−05 | 0.00016535 | 0.010659 | 279 | 4 | −2.364 | 0.99996 | 0.99996 | 1 | 17831 | 0 | −2.364 |
| NOC2L | 4 | 4.49E−05 | 0.00016535 | 0.010659 | 280 | 4 | −1.7436 | 0.99996 | 0.99996 | 1 | 17830 | 0 | −1.7436 |
| MRPL3 | 4 | 4.56E−05 | 0.0001659 | 0.010659 | 281 | 3 | −1.6549 | 0.78771 | 0.82056 | 1 | 13017 | 1 | −1.6549 |
| TOE1 | 4 | 4.73E−05 | 0.00017523 | 0.011165 | 282 | 4 | −0.91338 | 0.99995 | 0.99996 | 1 | 17829 | 0 | −0.91338 |
| OPA1 | 4 | 4.78E−05 | 0.00017632 | 0.011165 | 283 | 4 | −0.91645 | 0.99995 | 0.99996 | 1 | 17828 | 0 | −0.91645 |
| PAQR4 | 4 | 4.78E−05 | 0.00017632 | 0.011165 | 284 | 4 | −1.458 | 0.99995 | 0.99996 | 1 | 17827 | 0 | −1.458 |
| EXOSC4 | 4 | 4.79E−05 | 0.00017687 | 0.011165 | 285 | 4 | −1.9177 | 0.99995 | 0.99996 | 1 | 17826 | 0 | −1.9177 |
| ELP4 | 4 | 4.84E−05 | 0.00018071 | 0.011274 | 286 | 3 | −1.3753 | 0.89163 | 0.892 | 1 | 14307 | 1 | −1.3753 |
| GFER | 4 | 4.86E−05 | 0.00018181 | 0.011274 | 287 | 4 | −1.9132 | 0.99985 | 0.99986 | 1 | 17715 | 0 | −1.9132 |
| POT1 | 4 | 4.86E−05 | 0.00018181 | 0.011274 | 288 | 4 | −0.91801 | 0.99995 | 0.99996 | 1 | 17825 | 0 | −0.91801 |
| DARS2 | 4 | 4.87E−05 | 0.00018181 | 0.011274 | 289 | 4 | −2.0862 | 0.99941 | 0.99943 | 1 | 17540 | 0 | −2.0862 |
| SFXN4 | 4 | 4.88E−05 | 0.00018236 | 0.011274 | 290 | 3 | −1.2908 | 0.28437 | 0.47204 | 1 | 8185 | 1 | −1.2908 |
| EARS2 | 4 | 4.90E−05 | 0.00018236 | 0.011274 | 291 | 3 | −1.4401 | 0.48626 | 0.64167 | 1 | 10375 | 1 | −1.4401 |
| DHDDS | 4 | 4.98E−05 | 0.0001851 | 0.0114 | 292 | 4 | −1.8177 | 0.99991 | 0.99992 | 1 | 17763 | 0 | −1.8177 |
| CRCP | 4 | 4.99E−05 | 0.00018565 | 0.0114 | 293 | 4 | −1.1351 | 0.99995 | 0.99995 | 1 | 17824 | 0 | −1.1351 |
| ORC3 | 4 | 5.03E−05 | 0.00018784 | 0.01149 | 294 | 3 | −1.2192 | 0.6624 | 0.76355 | 1 | 12114 | 1 | −1.2192 |
| METTL16 | 4 | 5.08E−05 | 0.00018839 | 0.01149 | 295 | 4 | −1.8048 | 0.99995 | 0.99995 | 1 | 17823 | 0 | −1.8048 |
| TIMMDC1 | 4 | 5.12E−05 | 0.00019003 | 0.011551 | 296 | 4 | −1.5595 | 0.97396 | 0.9739 | 1 | 16160 | 0 | −1.5595 |
| CCT7 | 4 | 5.15E−05 | 0.00019168 | 0.011612 | 297 | 4 | −2.2605 | 0.99995 | 0.99995 | 1 | 17822 | 0 | −2.2605 |
| EEF2 | 4 | 5.27E−05 | 0.00019442 | 0.0117 | 298 | 4 | −3.2261 | 0.99995 | 0.99995 | 1 | 17821 | 0 | −3.2261 |
| TM2D3 | 4 | 5.30E−05 | 0.00019442 | 0.0117 | 299 | 4 | −0.64919 | 0.99995 | 0.99995 | 1 | 17820 | 0 | −0.64919 |
| DIS3 | 4 | 5.32E−05 | 0.00019607 | 0.011709 | 300 | 3 | −2.2503 | 0.28446 | 0.47213 | 1 | 8186 | 1 | −2.2503 |
| DNAJA3 | 4 | 5.33E−05 | 0.00019607 | 0.011709 | 301 | 4 | −1.8559 | 0.99995 | 0.99995 | 1 | 17819 | 0 | −1.8559 |
| OGT | 4 | 5.37E−05 | 0.00019716 | 0.011709 | 302 | 4 | −1.1892 | 0.97459 | 0.97457 | 1 | 16182 | 0 | −1.1892 |
| GEMIN5 | 4 | 5.38E−05 | 0.00019716 | 0.011709 | 303 | 4 | −1.3016 | 0.9997 | 0.99971 | 1 | 17641 | 0 | −1.3016 |
| CPSF4 | 4 | 5.47E−05 | 0.00020045 | 0.011865 | 304 | 4 | −1.2492 | 0.99995 | 0.99995 | 1 | 17817 | 0 | −1.2492 |
| COX11 | 4 | 5.49E−05 | 0.00020155 | 0.011891 | 305 | 4 | −1.6008 | 0.99936 | 0.99937 | 1 | 17522 | 0 | −1.6008 |
| DHX33 | 4 | 5.53E−05 | 0.0002032 | 0.011949 | 306 | 4 | −2.5133 | 0.99895 | 0.99895 | 1 | 17447 | 0 | −2.5133 |
| MRPL38 | 3 | 5.60E−05 | 0.00017687 | 0.011165 | 307 | 3 | −2.9301 | 0.9992 | 0.99921 | 1 | 17495 | 0 | −2.9301 |
| C1orf86 | 4 | 5.63E−05 | 0.00020649 | 0.012103 | 308 | 4 | −1.0327 | 0.99994 | 0.99995 | 1 | 17816 | 0 | −1.0327 |
| ALG13 | 4 | 5.66E−05 | 0.00020923 | 0.012224 | 309 | 3 | −2.4966 | 0.29915 | 0.48417 | 1 | 8353 | 1 | −2.4966 |
| PPP2R4 | 4 | 5.83E−05 | 0.00021471 | 0.012487 | 310 | 4 | −1.7288 | 0.99994 | 0.99995 | 1 | 17814 | 0 | −1.7288 |
| TMEM261 | 4 | 5.84E−05 | 0.00021526 | 0.012487 | 311 | 3 | −2.0217 | 0.3004 | 0.48526 | 1 | 8365 | 1 | −2.0217 |
| CLTC | 4 | 5.86E−05 | 0.00021581 | 0.012487 | 312 | 4 | −1.2127 | 0.99994 | 0.99995 | 1 | 17813 | 0 | −1.2127 |
| HEATR1 | 4 | 5.87E−05 | 0.00021691 | 0.012502 | 313 | 4 | −1.5377 | 0.99994 | 0.99995 | 1 | 17812 | 0 | −1.5377 |
| COX10 | 4 | 5.88E−05 | 0.00021746 | 0.012502 | 314 | 4 | −2.0747 | 0.99994 | 0.99995 | 1 | 17811 | 0 | −2.0747 |
| DSCC1 | 4 | 5.94E−05 | 0.00021855 | 0.012526 | 315 | 4 | −1.3243 | 0.99994 | 0.99995 | 1 | 17810 | 0 | −1.3243 |
| C7orf25 | 4 | 6.08E−05 | 0.00022404 | 0.012714 | 316 | 4 | −2.4123 | 0.99992 | 0.99993 | 1 | 17777 | 0 | −2.4123 |
| EXOC3 | 4 | 6.10E−05 | 0.00022513 | 0.012714 | 317 | 4 | −1.1459 | 0.99994 | 0.99995 | 1 | 17807 | 0 | −1.1459 |
| RARS2 | 4 | 6.11E−05 | 0.00022568 | 0.012714 | 318 | 4 | −2.3203 | 0.99994 | 0.99995 | 1 | 17806 | 0 | −2.3203 |
| TMEM258 | 4 | 6.12E−05 | 0.00022568 | 0.012714 | 319 | 4 | −1.7012 | 0.99994 | 0.99995 | 1 | 17805 | 0 | −1.7012 |
| ATP6V1C1 | 4 | 6.12E−05 | 0.00022623 | 0.012714 | 320 | 4 | −1.2541 | 0.99994 | 0.99995 | 1 | 17804 | 0 | −1.2541 |
| PITRM1 | 4 | 6.15E−05 | 0.00022623 | 0.012714 | 321 | 3 | −1.351 | 0.75944 | 0.8059 | 1 | 12760 | 1 | −1.351 |
| NDOR1 | 4 | 6.15E−05 | 0.00022678 | 0.012714 | 322 | 4 | −2.5959 | 0.99994 | 0.99995 | 1 | 17803 | 0 | −2.5959 |
| NDUFC1 | 4 | 6.19E−05 | 0.00022843 | 0.012767 | 323 | 4 | −1.5103 | 0.99994 | 0.99995 | 1 | 17802 | 0 | −1.5103 |
| FTSJ3 | 4 | 6.26E−05 | 0.00023336 | 0.013003 | 324 | 4 | −2.2844 | 0.99994 | 0.99995 | 1 | 17801 | 0 | −2.2844 |
| COASY | 4 | 6.31E−05 | 0.00023501 | 0.013054 | 325 | 4 | −1.7748 | 0.99994 | 0.99994 | 1 | 17800 | 0 | −1.7748 |
| TNPO1 | 4 | 6.44E−05 | 0.00024214 | 0.013345 | 326 | 4 | −1.5378 | 0.99637 | 0.99643 | 1 | 17155 | 0 | −1.5378 |
| CCDC115 | 4 | 6.45E−05 | 0.00024268 | 0.013345 | 327 | 4 | −1.4278 | 0.99994 | 0.99994 | 1 | 17798 | 0 | −1.4278 |
| PDSS2 | 4 | 6.46E−05 | 0.00024268 | 0.013345 | 328 | 4 | −1.4749 | 0.9993 | 0.99932 | 1 | 17513 | 0 | −1.4749 |
| RRP7A | 4 | 6.49E−05 | 0.00024433 | 0.013345 | 329 | 4 | −1.6871 | 0.98928 | 0.9894 | 1 | 16736 | 0 | −1.6871 |
| AURKB | 4 | 6.50E−05 | 0.00024488 | 0.013345 | 330 | 4 | −1.6988 | 0.99807 | 0.99811 | 1 | 17323 | 0 | −1.6988 |
| PI4KA | 4 | 6.51E−05 | 0.00024543 | 0.013345 | 331 | 4 | −1.5231 | 0.99993 | 0.99994 | 1 | 17797 | 0 | −1.5231 |
| CCT4 | 4 | 6.53E−05 | 0.00024543 | 0.013345 | 332 | 4 | −2.1462 | 0.99993 | 0.99994 | 1 | 17796 | 0 | −2.1462 |
| PDPK1 | 4 | 6.60E−05 | 0.00024707 | 0.013395 | 333 | 4 | −1.5361 | 0.99993 | 0.99994 | 1 | 17795 | 0 | −1.5361 |
| SLC31A1 | 4 | 6.62E−05 | 0.00024817 | 0.013403 | 334 | 4 | −1.0765 | 0.9926 | 0.99272 | 1 | 16915 | 0 | −1.0765 |
| OIP5 | 4 | 6.65E−05 | 0.00024872 | 0.013403 | 335 | 3 | −2.7142 | 0.010712 | 0.035679 | 0.536762 | 1182 | 1 | −2.7142 |
| NOL6 | 4 | 6.67E−05 | 0.00024981 | 0.013422 | 336 | 4 | −2.3027 | 0.99993 | 0.99994 | 1 | 17792 | 0 | −2.3027 |
| NELFA | 4 | 6.73E−05 | 0.00025201 | 0.0135 | 337 | 4 | −2.0258 | 0.99993 | 0.99994 | 1 | 17791 | 0 | −2.0258 |
| ACTR2 | 4 | 6.81E−05 | 0.0002542 | 0.013577 | 338 | 4 | −1.6288 | 0.99993 | 0.99994 | 1 | 17790 | 0 | −1.6288 |
| TOP3A | 4 | 6.88E−05 | 0.00025749 | 0.013712 | 339 | 3 | −1.7103 | 0.86432 | 0.86944 | 1 | 13889 | 1 | −1.7103 |
| DCST2 | 4 | 7.00E−05 | 0.00026023 | 0.013818 | 340 | 4 | −1.3843 | 0.99993 | 0.99994 | 1 | 17789 | 0 | −1.3843 |
| DPM3 | 4 | 7.03E−05 | 0.00026133 | 0.013835 | 341 | 4 | −0.93812 | 0.99993 | 0.99994 | 1 | 17788 | 0 | −0.93812 |
| PKN2 | 4 | 7.07E−05 | 0.00026353 | 0.013911 | 342 | 4 | −1.323 | 0.9833 | 0.98337 | 1 | 16473 | 0 | −1.323 |
| CENPM | 4 | 7.30E−05 | 0.00026791 | 0.014101 | 343 | 4 | −2.7542 | 0.99993 | 0.99994 | 1 | 17787 | 0 | −2.7542 |
| PHF6 | 4 | 7.36E−05 | 0.00027066 | 0.014191 | 344 | 4 | −0.95129 | 0.99993 | 0.99993 | 1 | 17786 | 0 | −0.95129 |
| RMND1 | 4 | 7.37E−05 | 0.0002712 | 0.014191 | 345 | 4 | −2.1857 | 0.99993 | 0.99993 | 1 | 17785 | 0 | −2.1857 |
| LEMD2 | 4 | 7.43E−05 | 0.0002734 | 0.014265 | 346 | 4 | −1.7855 | 0.99993 | 0.99993 | 1 | 17784 | 0 | −1.7855 |
| TSC2 | 4 | 7.46E−05 | 0.00027559 | 0.014338 | 347 | 4 | −1.2936 | 0.99993 | 0.99993 | 1 | 17783 | 0 | −1.2936 |
| LIAS | 4 | 7.56E−05 | 0.00028162 | 0.01461 | 348 | 4 | −1.7787 | 0.99992 | 0.99993 | 1 | 17782 | 0 | −1.7787 |
| TEX10 | 4 | 7.64E−05 | 0.00028382 | 0.014639 | 349 | 4 | −1.2531 | 0.99992 | 0.99993 | 1 | 17781 | 0 | −1.2531 |
| IPO9 | 4 | 7.64E−05 | 0.00028382 | 0.014639 | 350 | 3 | −1.5514 | 0.10882 | 0.23857 | 0.86829 | 4906 | 1 | −1.5514 |
| GNB2L1 | 4 | 7.66E−05 | 0.00028491 | 0.014654 | 351 | 4 | −1.4387 | 0.99989 | 0.99989 | 1 | 17742 | 0 | −1.4387 |
| POLR3C | 4 | 7.73E−05 | 0.00028766 | 0.014739 | 352 | 4 | −2.1096 | 0.99992 | 0.99993 | 1 | 17780 | 0 | −2.1096 |
| PHB | 4 | 7.74E−05 | 0.00028821 | 0.014739 | 353 | 4 | −0.66377 | 0.99992 | 0.99993 | 1 | 17779 | 0 | −0.66377 |
| TRAPPC11 | 4 | 7.86E−05 | 0.00029314 | 0.014949 | 354 | 4 | −1.3146 | 0.99992 | 0.99993 | 1 | 17778 | 0 | −1.3146 |
| ATP6V1F | 4 | 8.01E−05 | 0.00030027 | 0.01527 | 355 | 4 | −1.3822 | 0.97957 | 0.97966 | 1 | 16343 | 0 | −1.3822 |
| RBFA | 4 | 8.09E−05 | 0.00030301 | 0.015366 | 356 | 4 | −1.2488 | 0.99992 | 0.99992 | 1 | 17776 | 0 | −1.2488 |
| PIK3C3 | 4 | 8.12E−05 | 0.00030411 | 0.015378 | 357 | 4 | −1.3101 | 0.99992 | 0.99992 | 1 | 17775 | 0 | −1.3101 |
| SS18L2 | 4 | 8.18E−05 | 0.0003063 | 0.015403 | 358 | 4 | −1.8054 | 0.99992 | 0.99992 | 1 | 17774 | 0 | −1.8054 |
| G6PD | 4 | 8.19E−05 | 0.0003063 | 0.015403 | 359 | 3 | −2.0605 | 0.79197 | 0.82288 | 1 | 13056 | 1 | −2.0605 |
| COX6B1 | 4 | 8.24E−05 | 0.0003074 | 0.015415 | 360 | 4 | −1.1999 | 0.99992 | 0.99992 | 1 | 17772 | 0 | −1.1999 |
| GMPS | 4 | 8.31E−05 | 0.00030959 | 0.015482 | 361 | 4 | −1.5348 | 0.99992 | 0.99992 | 1 | 17771 | 0 | −1.5348 |
| LARS | 4 | 8.49E−05 | 0.00031453 | 0.015642 | 362 | 4 | −2.0903 | 0.99992 | 0.99992 | 1 | 17770 | 0 | −2.0903 |
| RBM15 | 4 | 8.52E−05 | 0.00031453 | 0.015642 | 363 | 4 | −0.91279 | 0.99991 | 0.99992 | 1 | 17769 | 0 | −0.91279 |
| RSBN1 | 4 | 8.64E−05 | 0.00031782 | 0.015763 | 364 | 4 | −1.1801 | 0.99991 | 0.99992 | 1 | 17768 | 0 | −1.1801 |
| EXOSC2 | 4 | 8.76E−05 | 0.00032056 | 0.015839 | 365 | 4 | −2.2014 | 0.99877 | 0.99877 | 1 | 17420 | 0 | −2.2014 |
| HARS2 | 4 | 8.78E−05 | 0.00032111 | 0.015839 | 366 | 4 | −1.9989 | 0.99991 | 0.99992 | 1 | 17767 | 0 | −1.9989 |
| RPF2 | 4 | 8.82E−05 | 0.00032331 | 0.015904 | 367 | 4 | −1.6299 | 0.99991 | 0.99992 | 1 | 17766 | 0 | −1.6299 |
| SORT1 | 4 | 8.88E−05 | 0.00032605 | 0.015995 | 368 | 4 | −0.79194 | 0.99991 | 0.99992 | 1 | 17765 | 0 | −0.79194 |
| CD3EAP | 4 | 8.93E−05 | 0.00032714 | 0.016005 | 369 | 4 | −0.91376 | 0.99991 | 0.99992 | 1 | 17764 | 0 | −0.91376 |
| GATA5 | 4 | 9.19E−05 | 0.00033921 | 0.016551 | 370 | 4 | −0.73925 | 0.99991 | 0.99991 | 1 | 17762 | 0 | −0.73925 |
| DENR | 4 | 9.24E−05 | 0.00034058 | 0.016573 | 371 | 3 | −1.046 | 0.66395 | 0.76414 | 1 | 12129 | 1 | −1.046 |
| C18orf21 | 4 | 9.35E−05 | 0.00034415 | 0.016656 | 372 | 4 | −1.7774 | 0.99983 | 0.99984 | 1 | 17700 | 0 | −1.7774 |
| C7orf26 | 4 | 9.36E−05 | 0.00034415 | 0.016656 | 373 | 4 | −1.2403 | 0.99991 | 0.99991 | 1 | 17761 | 0 | −1.2403 |
| PTCD3 | 4 | 9.51E−05 | 0.00035182 | 0.016983 | 374 | 4 | −1.5106 | 0.9999 | 0.99991 | 1 | 17760 | 0 | −1.5106 |
| PRMT1 | 4 | 9.60E−05 | 0.00035566 | 0.017103 | 375 | 3 | −2.4486 | 0.90683 | 0.90654 | 1 | 14571 | 0 | −2.4486 |
| NDUFAF4 | 4 | 9.62E−05 | 0.00035621 | 0.017103 | 376 | 3 | −2.8677 | 0.89782 | 0.89777 | 1 | 14404 | 1 | −2.8677 |
| EGLN1 | 4 | 9.64E−05 | 0.00035731 | 0.017104 | 377 | 4 | −1.2972 | 0.9999 | 0.99991 | 1 | 17759 | 0 | −1.2972 |
| CS | 4 | 9.66E−05 | 0.00035813 | 0.017104 | 378 | 4 | −0.94822 | 0.9999 | 0.99991 | 1 | 17758 | 0 | −0.94822 |
| MTERFD1 | 4 | 9.67E−05 | 0.00035923 | 0.017111 | 379 | 4 | −0.84782 | 0.9999 | 0.99991 | 1 | 17757 | 0 | −0.84782 |
| C12orf45 | 4 | 9.73E−05 | 0.00036115 | 0.017157 | 380 | 4 | −0.8919 | 0.9999 | 0.99991 | 1 | 17756 | 0 | −0.8919 |
| OXA1L | 4 | 9.84E−05 | 0.00036608 | 0.017346 | 381 | 4 | −1.0359 | 0.9999 | 0.99991 | 1 | 17755 | 0 | −1.0359 |
| AP2M1 | 4 | 9.89E−05 | 0.00036718 | 0.017352 | 382 | 3 | −1.5896 | 0.48437 | 0.64008 | 1 | 10349 | 1 | −1.5896 |
| MCAT | 4 | 9.96E−05 | 0.00036992 | 0.017352 | 383 | 4 | −1.6999 | 0.97851 | 0.9786 | 1 | 16315 | 0 | −1.6999 |
| SYS1 | 4 | 9.97E−05 | 0.00036992 | 0.017352 | 384 | 4 | −1.4135 | 0.9999 | 0.99991 | 1 | 17754 | 0 | −1.4135 |
| KIAA1324 | 4 | 0.00010008 | 0.00037047 | 0.017352 | 385 | 4 | −0.90919 | 0.9999 | 0.99991 | 1 | 17753 | 0 | −0.90919 |
| FASTKD5 | 4 | 0.00010032 | 0.00037102 | 0.017352 | 386 | 4 | −1.4304 | 0.99984 | 0.99985 | 1 | 17705 | 0 | −1.4304 |
| MRPL2 | 4 | 0.00010048 | 0.00037212 | 0.017359 | 387 | 4 | −1.5188 | 0.9999 | 0.99991 | 1 | 17752 | 0 | −1.5188 |
| CAPZA1 | 4 | 0.00010156 | 0.0003765 | 0.017518 | 388 | 4 | −0.83232 | 0.9999 | 0.9999 | 1 | 17751 | 0 | −0.83232 |
| NHP2 | 4 | 0.00010206 | 0.00037815 | 0.017549 | 389 | 4 | −2.4304 | 0.99957 | 0.99959 | 1 | 17597 | 0 | −2.4304 |
| MMGT1 | 4 | 0.00010276 | 0.00038144 | 0.017657 | 390 | 4 | −1.1768 | 0.9999 | 0.9999 | 1 | 17750 | 0 | −1.1768 |
| RNMTL1 | 4 | 0.00010365 | 0.00038473 | 0.017698 | 391 | 3 | −1.8816 | 0.95276 | 0.9526 | 1 | 15555 | 0 | −1.8816 |
| MTFMT | 4 | 0.00010392 | 0.00038528 | 0.017698 | 392 | 4 | −1.4999 | 0.9999 | 0.9999 | 1 | 17749 | 0 | −1.4999 |
| CARD10 | 4 | 0.00010398 | 0.00038528 | 0.017698 | 393 | 4 | −0.87448 | 0.9999 | 0.9999 | 1 | 17748 | 0 | −0.87448 |
| VMP1 | 4 | 0.00010468 | 0.00038692 | 0.017729 | 394 | 4 | −2.2825 | 0.99943 | 0.99945 | 1 | 17547 | 0 | −2.2825 |
| FBL | 4 | 0.00010557 | 0.00039131 | 0.017884 | 395 | 4 | −2.1994 | 0.99986 | 0.99987 | 1 | 17722 | 0 | −2.1994 |
| SNRPA | 4 | 0.00010608 | 0.0003946 | 0.017989 | 396 | 4 | −0.85298 | 0.99989 | 0.9999 | 1 | 17747 | 0 | −0.85298 |
| TAF2 | 4 | 0.00010674 | 0.00039899 | 0.018144 | 397 | 4 | −1.6548 | 0.99989 | 0.9999 | 1 | 17746 | 0 | −1.6548 |
| FXN | 4 | 0.00010852 | 0.00040228 | 0.018226 | 398 | 4 | −2.3715 | 0.99989 | 0.99989 | 1 | 17745 | 0 | −2.3715 |
| DHPS | 4 | 0.00010885 | 0.00040283 | 0.018226 | 399 | 4 | −1.8131 | 0.99864 | 0.99865 | 1 | 17399 | 0 | −1.8131 |
| NDUFS2 | 4 | 0.00010925 | 0.00040393 | 0.01823 | 400 | 4 | −1.6602 | 0.99989 | 0.99989 | 1 | 17744 | 0 | −1.6602 |
| PIGM | 4 | 0.00011119 | 0.00040996 | 0.018456 | 401 | 4 | −0.97611 | 0.99989 | 0.99989 | 1 | 17743 | 0 | −0.97611 |
| CFDP1 | 4 | 0.0001122 | 0.00041599 | 0.018681 | 402 | 4 | −1.4115 | 0.99347 | 0.99358 | 1 | 16959 | 0 | −1.4115 |
| TRMT6 | 4 | 0.00011453 | 0.00042806 | 0.019175 | 403 | 3 | −1.3112 | 0.48169 | 0.6378 | 1 | 10324 | 1 | −1.3112 |
| GTF2H3 | 4 | 0.00011503 | 0.0004308 | 0.019215 | 404 | 4 | −0.8448 | 0.99988 | 0.99989 | 1 | 17740 | 0 | −0.8448 |
| YARS | 4 | 0.00011515 | 0.00043107 | 0.019215 | 405 | 4 | −2.5691 | 0.99988 | 0.99989 | 1 | 17739 | 0 | −2.5691 |
| LRPPRC | 4 | 0.00011689 | 0.00043903 | 0.019522 | 406 | 3 | −1.3022 | 0.96035 | 0.96022 | 1 | 15754 | 0 | −1.3022 |
| NUP50 | 4 | 0.00012149 | 0.00046151 | 0.020471 | 407 | 4 | −1.7194 | 0.99988 | 0.99988 | 1 | 17738 | 0 | −1.7194 |
| TRAPPC3 | 4 | 0.00012186 | 0.00046316 | 0.020494 | 408 | 3 | −1.2908 | 0.82065 | 0.83958 | 1 | 13362 | 1 | −1.2908 |
| PELO | 4 | 0.00012446 | 0.00047248 | 0.020855 | 409 | 4 | −1.8232 | 0.99988 | 0.99988 | 1 | 17737 | 0 | −1.8232 |
| SDHC | 4 | 0.0001258 | 0.00047851 | 0.021043 | 410 | 4 | −1.0873 | 0.99557 | 0.99566 | 1 | 17098 | 0 | −1.0873 |
| KREMEN2 | 4 | 0.00012586 | 0.00047906 | 0.021043 | 411 | 4 | −1.0026 | 0.99987 | 0.99988 | 1 | 17736 | 0 | −1.0026 |
| NGDN | 4 | 0.00012721 | 0.00048455 | 0.021232 | 412 | 4 | −1.6157 | 0.99987 | 0.99988 | 1 | 17735 | 0 | −1.6157 |
| SLC30A9 | 4 | 0.0001283 | 0.00048674 | 0.021276 | 413 | 4 | −0.8169 | 0.99987 | 0.99988 | 1 | 17734 | 0 | −0.8169 |
| MRPS14 | 4 | 0.00012864 | 0.00048948 | 0.021345 | 414 | 4 | −2.7074 | 0.99987 | 0.99988 | 1 | 17733 | 0 | −2.7074 |
| SMNDC1 | 4 | 0.00012925 | 0.00049332 | 0.021428 | 415 | 4 | −1.605 | 0.99987 | 0.99988 | 1 | 17732 | 0 | −1.605 |
| BUB1 | 4 | 0.00012973 | 0.00049497 | 0.021428 | 416 | 4 | −1.3761 | 0.99987 | 0.99988 | 1 | 17731 | 0 | −1.3761 |
| YKT6 | 4 | 0.0001298 | 0.00049497 | 0.021428 | 417 | 4 | −1.2993 | 0.99987 | 0.99988 | 1 | 17730 | 0 | −1.2993 |
| RPS2 | 4 | 0.00013028 | 0.00049771 | 0.021496 | 418 | 4 | −1.4859 | 0.99987 | 0.99988 | 1 | 17729 | 0 | −1.4859 |
| PAXIP1 | 4 | 0.00013118 | 0.00050374 | 0.021704 | 419 | 4 | −1.2046 | 0.99987 | 0.99987 | 1 | 17728 | 0 | −1.2046 |
| DDX52 | 4 | 0.00013153 | 0.00050539 | 0.021723 | 420 | 3 | −1.2486 | 0.88015 | 0.88192 | 1 | 14131 | 1 | −1.2486 |
| RABGGTA | 4 | 0.00013236 | 0.00050868 | 0.021813 | 421 | 4 | −1.5175 | 0.99987 | 0.99987 | 1 | 17727 | 0 | −1.5175 |
| PLEC | 4 | 0.00013602 | 0.00052239 | 0.022295 | 422 | 4 | −0.92609 | 0.99986 | 0.99987 | 1 | 17726 | 0 | −0.92609 |
| HIRA | 4 | 0.00013616 | 0.00052239 | 0.022295 | 423 | 4 | −1.0969 | 0.99986 | 0.99987 | 1 | 17725 | 0 | −1.0969 |
| TFRC | 4 | 0.00013702 | 0.00052458 | 0.0223 | 424 | 4 | −0.76405 | 0.99986 | 0.99987 | 1 | 17724 | 0 | −0.76405 |
| COQ6 | 4 | 0.00013759 | 0.00052623 | 0.0223 | 425 | 4 | −0.90155 | 0.99986 | 0.99987 | 1 | 17723 | 0 | −0.90155 |
| MVD | 4 | 0.00013769 | 0.00052623 | 0.0223 | 426 | 3 | −2.2878 | 0.48513 | 0.64072 | 1 | 10356 | 1 | −2.2878 |
| GTF3C1 | 4 | 0.00014009 | 0.00053336 | 0.02255 | 427 | 4 | −1.8687 | 0.99014 | 0.99022 | 1 | 16779 | 0 | −1.8687 |
| MRPS11 | 4 | 0.00014107 | 0.00053665 | 0.022636 | 428 | 4 | −2.1012 | 0.99986 | 0.99987 | 1 | 17721 | 0 | −2.1012 |
| XRCC3 | 4 | 0.00014188 | 0.00054049 | 0.022745 | 429 | 4 | −1.7596 | 0.99317 | 0.99328 | 1 | 16941 | 0 | −1.7596 |
| ATP6V1H | 4 | 0.00014314 | 0.00054652 | 0.022945 | 430 | 3 | −1.2922 | 0.95672 | 0.9566 | 1 | 15660 | 0 | −1.2922 |
| ELP2 | 4 | 0.0001455 | 0.0005553 | 0.02325 | 431 | 4 | −0.96429 | 0.99985 | 0.99986 | 1 | 17720 | 0 | −0.96429 |
| TAMM41 | 4 | 0.00014587 | 0.00055639 | 0.02325 | 432 | 4 | −1.389 | 0.99891 | 0.9989 | 1 | 17440 | 0 | −1.389 |
| MPHOSPH10 | 4 | 0.00014648 | 0.00055859 | 0.02325 | 433 | 4 | −1.2081 | 0.99985 | 0.99986 | 1 | 17719 | 0 | −1.2081 |
| ATP6V0C | 4 | 0.00014715 | 0.00056023 | 0.02325 | 434 | 4 | −2.4937 | 0.9994 | 0.99942 | 1 | 17537 | 0 | −2.4937 |
| RNGTT | 4 | 0.00014723 | 0.00056023 | 0.02325 | 435 | 4 | −1.4545 | 0.99985 | 0.99986 | 1 | 17718 | 0 | −1.4545 |
| SDHB | 4 | 0.00014789 | 0.00056352 | 0.023333 | 436 | 4 | −1.352 | 0.9995 | 0.99952 | 1 | 17566 | 0 | −1.352 |
| RRP36 | 4 | 0.0001489 | 0.00056626 | 0.023393 | 437 | 4 | −1.7306 | 0.99985 | 0.99986 | 1 | 17717 | 0 | −1.7306 |
| PFDN2 | 4 | 0.00015096 | 0.00057559 | 0.023684 | 438 | 4 | −1.6271 | 0.99985 | 0.99986 | 1 | 17716 | 0 | −1.6271 |
| HSPA13 | 4 | 0.00015105 | 0.00057614 | 0.023684 | 439 | 4 | −1.2857 | 0.99899 | 0.99898 | 1 | 17456 | 0 | −1.2857 |
| TIMM10 | 4 | 0.00015124 | 0.00057723 | 0.023684 | 440 | 4 | −2.5665 | 0.99977 | 0.99977 | 1 | 17666 | 0 | −2.5665 |
| TRPM7 | 4 | 0.00015237 | 0.00058272 | 0.023854 | 441 | 3 | −1.5785 | 0.91446 | 0.91425 | 1 | 14713 | 0 | −1.5785 |
| STIL | 4 | 0.00015359 | 0.00058875 | 0.024047 | 442 | 4 | −1.0712 | 0.99985 | 0.99986 | 1 | 17714 | 0 | −1.0712 |
| UBR4 | 4 | 0.00015634 | 0.00059807 | 0.024373 | 443 | 4 | −1.7509 | 0.99984 | 0.99986 | 1 | 17712 | 0 | −1.7509 |
| EXOSC10 | 4 | 0.00015809 | 0.00060136 | 0.024398 | 444 | 4 | −0.9147 | 0.99723 | 0.99726 | 1 | 17227 | 0 | −0.9147 |
| MTIF3 | 4 | 0.0001588 | 0.00060301 | 0.024398 | 445 | 4 | −0.91419 | 0.99984 | 0.99985 | 1 | 17711 | 0 | −0.91419 |
| SH3GL1 | 4 | 0.00015896 | 0.00060356 | 0.024398 | 446 | 4 | −0.99035 | 0.99984 | 0.99985 | 1 | 17710 | 0 | −0.99035 |
| AMIGO1 | 4 | 0.00015945 | 0.00060411 | 0.024398 | 447 | 3 | −1.0607 | 0.26847 | 0.45892 | 1 | 8034 | 1 | −1.0607 |
| GPI | 4 | 0.00016008 | 0.00060795 | 0.024498 | 448 | 4 | −0.88974 | 0.99984 | 0.99985 | 1 | 17709 | 0 | −0.88974 |
| KRR1 | 4 | 0.00016159 | 0.00061178 | 0.024598 | 449 | 3 | −1.6304 | 0.64965 | 0.75879 | 1 | 12039 | 1 | −1.6304 |
| EXOC5 | 4 | 0.00016348 | 0.00062111 | 0.024917 | 450 | 4 | −1.305 | 0.99984 | 0.99985 | 1 | 17707 | 0 | −1.305 |
| ATP5A1 | 4 | 0.00016397 | 0.00062385 | 0.024949 | 451 | 4 | −1.9675 | 0.99984 | 0.99985 | 1 | 17706 | 0 | −1.9675 |
| KIAA1211 | 4 | 0.00016487 | 0.0006255 | 0.024949 | 452 | 4 | −1.0111 | 0.99984 | 0.99985 | 1 | 17704 | 0 | −1.0111 |
| NUP188 | 4 | 0.00016504 | 0.00062604 | 0.024949 | 453 | 4 | −1.0682 | 0.99983 | 0.99985 | 1 | 17703 | 0 | −1.0682 |
| CYC1 | 4 | 0.00016977 | 0.00064359 | 0.025579 | 454 | 4 | −1.4647 | 0.99983 | 0.99984 | 1 | 17702 | 0 | −1.4647 |
| IMP4 | 4 | 0.0001702 | 0.00064469 | 0.025579 | 455 | 4 | −1.697 | 0.99983 | 0.99984 | 1 | 17701 | 0 | −1.697 |
| TMED2 | 4 | 0.00017197 | 0.00065127 | 0.025736 | 456 | 4 | −1.3033 | 0.99983 | 0.99984 | 1 | 17699 | 0 | −1.3033 |
| OGFR | 4 | 0.00017222 | 0.00065182 | 0.025736 | 457 | 4 | −0.88492 | 0.99983 | 0.99984 | 1 | 17698 | 0 | −0.88492 |
| TMA16 | 4 | 0.00017281 | 0.00065292 | 0.025736 | 458 | 4 | −1.4056 | 0.99855 | 0.99856 | 1 | 17380 | 0 | −1.4056 |
| MRPL37 | 4 | 0.00017418 | 0.00065731 | 0.025853 | 459 | 4 | −1.4751 | 0.99983 | 0.99984 | 1 | 17697 | 0 | −1.4751 |
| NELFCD | 4 | 0.00017513 | 0.0006606 | 0.025926 | 460 | 4 | −1.1479 | 0.99982 | 0.99984 | 1 | 17696 | 0 | −1.1479 |
| XRCC6 | 4 | 0.00017611 | 0.00066389 | 0.025953 | 461 | 4 | −1.879 | 0.99365 | 0.99375 | 1 | 16970 | 0 | −1.879 |
| HDAC8 | 4 | 0.00017625 | 0.00066416 | 0.025953 | 462 | 4 | −2.1149 | 0.99982 | 0.99984 | 1 | 17695 | 0 | −2.1149 |
| BGLAP | 4 | 0.00017842 | 0.00067211 | 0.026096 | 463 | 4 | −1.0547 | 0.99982 | 0.99983 | 1 | 17694 | 0 | −1.0547 |
| EIF2B1 | 4 | 0.00017859 | 0.00067266 | 0.026096 | 464 | 4 | −1.516 | 0.99982 | 0.99983 | 1 | 17693 | 0 | −1.516 |
| VPS13D | 4 | 0.00017885 | 0.00067431 | 0.026096 | 465 | 4 | −0.8121 | 0.99982 | 0.99983 | 1 | 17692 | 0 | −0.8121 |
| RIOK1 | 4 | 0.00017903 | 0.00067431 | 0.026096 | 466 | 4 | −1.2715 | 0.9793 | 0.97939 | 1 | 16333 | 0 | −1.2715 |
| GPX4 | 4 | 0.00017945 | 0.0006754 | 0.026096 | 467 | 4 | −2.4 | 0.99655 | 0.99659 | 1 | 17169 | 0 | −2.4 |
| RFT1 | 4 | 0.00018017 | 0.0006765 | 0.026096 | 468 | 4 | −1.7497 | 0.99982 | 0.99983 | 1 | 17691 | 0 | −1.7497 |
| CYP2W1 | 4 | 0.00018104 | 0.00068089 | 0.026209 | 469 | 4 | −1.2769 | 0.99982 | 0.99983 | 1 | 17690 | 0 | −1.2769 |
| ATP5O | 4 | 0.00018246 | 0.00068692 | 0.026385 | 470 | 4 | −2.4083 | 0.99982 | 0.99983 | 1 | 17689 | 0 | −2.4083 |
| LSM12 | 4 | 0.00018326 | 0.00069076 | 0.026476 | 471 | 4 | −0.97689 | 0.99982 | 0.99982 | 1 | 17688 | 0 | −0.97689 |
| SLC6A17 | 4 | 0.00018411 | 0.0006957 | 0.026609 | 472 | 4 | −1.1101 | 0.99892 | 0.9989 | 1 | 17442 | 0 | −1.1101 |
| NFU1 | 4 | 0.00018798 | 0.00070831 | 0.026941 | 473 | 3 | −2.2678 | 0.042577 | 0.12328 | 0.704716 | 3116 | 1 | −2.2678 |
| TTI1 | 4 | 0.00018802 | 0.00070831 | 0.026941 | 474 | 4 | −1.4107 | 0.99981 | 0.99982 | 1 | 17687 | 0 | −1.4107 |
| ATP6V0B | 4 | 0.00018841 | 0.00070886 | 0.026941 | 475 | 4 | −1.5321 | 0.96956 | 0.96947 | 1 | 16011 | 0 | −1.5321 |
| THBS3 | 4 | 0.00019103 | 0.00071763 | 0.02716 | 476 | 4 | −0.9393 | 0.99981 | 0.99982 | 1 | 17686 | 0 | −0.9393 |
| NDUFB6 | 4 | 0.00019344 | 0.00072476 | 0.027373 | 477 | 3 | −1.6856 | 0.8655 | 0.87035 | 1 | 13907 | 1 | −1.6856 |
| DDX56 | 4 | 0.00019556 | 0.00073573 | 0.027729 | 478 | 4 | −1.5255 | 0.9998 | 0.99981 | 1 | 17685 | 0 | −1.5255 |
| FTSJ2 | 4 | 0.0001961 | 0.00073902 | 0.027795 | 479 | 4 | −2.4374 | 0.9984 | 0.99843 | 1 | 17361 | 0 | −2.4374 |
| LSM10 | 4 | 0.00019792 | 0.00074396 | 0.027922 | 480 | 3 | −1.9884 | 0.85838 | 0.86502 | 1 | 13822 | 1 | −1.9884 |
| UTP14A | 4 | 0.00019875 | 0.00074615 | 0.027947 | 481 | 4 | −0.93919 | 0.9998 | 0.99981 | 1 | 17684 | 0 | −0.93919 |
| DGCR8 | 4 | 0.00020036 | 0.00075219 | 0.028114 | 482 | 4 | −1.6441 | 0.99977 | 0.99978 | 1 | 17667 | 0 | −1.6441 |
| RPS19BP1 | 4 | 0.00020141 | 0.00075548 | 0.028179 | 483 | 4 | −2.2346 | 0.9998 | 0.99981 | 1 | 17683 | 0 | −2.2346 |
| FBXW11 | 4 | 0.00020217 | 0.00075877 | 0.028226 | 484 | 4 | −1.5381 | 0.99293 | 0.99305 | 1 | 16931 | 0 | −1.5381 |
| SRP19 | 4 | 0.00020263 | 0.00075986 | 0.028226 | 485 | 4 | −2.4973 | 0.99953 | 0.99955 | 1 | 17581 | 0 | −2.4973 |
| SPCS3 | 4 | 0.00020594 | 0.00076919 | 0.028385 | 486 | 4 | −1.4489 | 0.99979 | 0.99981 | 1 | 17681 | 0 | −1.4489 |
| WRAP53 | 4 | 0.00020662 | 0.00077193 | 0.028385 | 487 | 4 | −1.0689 | 0.99979 | 0.99981 | 1 | 17680 | 0 | −1.0689 |
| C16orf59 | 4 | 0.00020701 | 0.00077248 | 0.028385 | 488 | 4 | −1.215 | 0.99979 | 0.9998 | 1 | 17679 | 0 | −1.215 |
| HGS | 4 | 0.00020749 | 0.00077248 | 0.028385 | 489 | 4 | −1.2053 | 0.99979 | 0.9998 | 1 | 17678 | 0 | −1.2053 |
| MRPL45 | 4 | 0.00020767 | 0.00077303 | 0.028385 | 490 | 3 | −2.4867 | 0.87818 | 0.88034 | 1 | 14092 | 1 | −2.4867 |
| SRCAP | 4 | 0.00020789 | 0.00077357 | 0.028385 | 491 | 4 | −1.7247 | 0.99979 | 0.9998 | 1 | 17677 | 0 | −1.7247 |
| POLG | 4 | 0.00020868 | 0.00077741 | 0.028468 | 492 | 3 | −1.145 | 0.0023972 | 0.008574 | 0.407201 | 377 | 1 | −1.145 |
| CDK7 | 4 | 0.00021093 | 0.00078564 | 0.028711 | 493 | 4 | −1.82 | 0.99606 | 0.99615 | 1 | 17132 | 0 | −1.82 |
| THOC5 | 4 | 0.00021143 | 0.00078729 | 0.028713 | 494 | 4 | −0.95297 | 0.99979 | 0.9998 | 1 | 17676 | 0 | −0.95297 |
| ZC3H8 | 4 | 0.00021441 | 0.00079606 | 0.028974 | 495 | 4 | −1.7251 | 0.99979 | 0.99979 | 1 | 17674 | 0 | −1.7251 |
| IPO7 | 4 | 0.00021551 | 0.0007988 | 0.028997 | 496 | 4 | −1.225 | 0.99978 | 0.99979 | 1 | 17673 | 0 | −1.225 |
| ATP6AP2 | 4 | 0.00021587 | 0.0007999 | 0.028997 | 497 | 4 | −1.4761 | 0.99953 | 0.99955 | 1 | 17585 | 0 | −1.4761 |
| WDR61 | 4 | 0.00021801 | 0.00080758 | 0.029217 | 498 | 4 | −1.9499 | 0.99916 | 0.99915 | 1 | 17489 | 0 | −1.9499 |
| MRPS7 | 4 | 0.00021844 | 0.00081087 | 0.029277 | 499 | 4 | −1.4405 | 0.99978 | 0.99979 | 1 | 17671 | 0 | −1.4405 |
| WDR1 | 4 | 0.00021976 | 0.00081416 | 0.029337 | 500 | 4 | −1.6793 | 0.99978 | 0.99979 | 1 | 17670 | 0 | −1.6793 |
| NOP9 | 4 | 0.00022335 | 0.00082732 | 0.029752 | 501 | 4 | −1.5862 | 0.99978 | 0.99978 | 1 | 17669 | 0 | −1.5862 |
| XRCC5 | 4 | 0.00022397 | 0.00083116 | 0.029831 | 502 | 4 | −1.9253 | 0.99978 | 0.99978 | 1 | 17668 | 0 | −1.9253 |
| SCO1 | 4 | 0.00023365 | 0.00086845 | 0.031058 | 503 | 4 | −1.2304 | 0.99899 | 0.99897 | 1 | 17454 | 0 | −1.2304 |
| GPATCH1 | 4 | 0.0002344 | 0.0008712 | 0.031058 | 504 | 3 | −1.2556 | 0.93875 | 0.93862 | 1 | 15220 | 0 | −1.2556 |
| RAD9A | 4 | 0.0002347 | 0.00087175 | 0.031058 | 505 | 4 | −2.0188 | 0.99977 | 0.99977 | 1 | 17665 | 0 | −2.0188 |
| RBBP8NL | 4 | 0.00023513 | 0.00087394 | 0.031058 | 506 | 4 | −0.94143 | 0.99976 | 0.99977 | 1 | 17664 | 0 | −0.94143 |
| TARDBP | 4 | 0.00023524 | 0.00087394 | 0.031058 | 507 | 4 | −1.6415 | 0.99976 | 0.99977 | 1 | 17663 | 0 | −1.6415 |
| DHX35 | 4 | 0.00023674 | 0.00087723 | 0.031072 | 508 | 4 | −1.5402 | 0.99976 | 0.99977 | 1 | 17662 | 0 | −1.5402 |
| LCMT1 | 4 | 0.00023696 | 0.00087778 | 0.031072 | 509 | 4 | −1.1028 | 0.99976 | 0.99977 | 1 | 17661 | 0 | −1.1028 |
| ETV3L | 4 | 0.00024122 | 0.00089094 | 0.031439 | 510 | 3 | −0.78941 | 0.88908 | 0.88965 | 1 | 14267 | 1 | −0.78941 |
| POP7 | 4 | 0.00024199 | 0.00089259 | 0.031439 | 511 | 3 | −1.8852 | 0.77875 | 0.81574 | 1 | 12942 | 1 | −1.8852 |
| EXOSC8 | 4 | 0.0002425 | 0.00089368 | 0.031439 | 512 | 4 | −1.4322 | 0.99963 | 0.99964 | 1 | 17617 | 0 | −1.4322 |
| BRD4 | 4 | 0.00024404 | 0.00089752 | 0.031439 | 513 | 3 | −1.0642 | 0.5804 | 0.72255 | 1 | 11508 | 1 | −1.0642 |
| CD1D | 4 | 0.00024426 | 0.00089862 | 0.031439 | 514 | 4 | −0.77425 | 0.99976 | 0.99976 | 1 | 17660 | 0 | −0.77425 |
| EMC3 | 4 | 0.0002443 | 0.00089862 | 0.031439 | 515 | 3 | −1.1338 | 0.12123 | 0.25887 | 0.885073 | 5220 | 1 | −1.1338 |
| TRAPPC4 | 4 | 0.00025447 | 0.0009329 | 0.032576 | 516 | 4 | −1.8552 | 0.99625 | 0.99633 | 1 | 17147 | 0 | −1.8552 |
| MRPL51 | 4 | 0.00025548 | 0.00093701 | 0.032626 | 517 | 4 | −1.4904 | 0.99974 | 0.99975 | 1 | 17659 | 0 | −1.4904 |
| CSNK2B | 4 | 0.00025617 | 0.00093866 | 0.032626 | 518 | 4 | −1.8737 | 0.99974 | 0.99975 | 1 | 17658 | 0 | −1.8737 |
| LSG1 | 4 | 0.00025659 | 0.00093975 | 0.032626 | 519 | 3 | −1.2888 | 0.35378 | 0.52985 | 1 | 8940 | 1 | −1.2888 |
| TPI1 | 4 | 0.00025743 | 0.00094195 | 0.032639 | 520 | 4 | −1.3728 | 0.99974 | 0.99975 | 1 | 17657 | 0 | −1.3728 |
| PRPF38B | 4 | 0.00025789 | 0.00094469 | 0.032671 | 521 | 4 | −2.5334 | 0.99974 | 0.99975 | 1 | 17656 | 0 | −2.5334 |
| AURKAIP1 | 4 | 0.0002606 | 0.00095621 | 0.032943 | 522 | 4 | −1.4286 | 0.99643 | 0.9965 | 1 | 17159 | 0 | −1.4286 |
| LIN52 | 4 | 0.00026066 | 0.00095621 | 0.032943 | 523 | 4 | −1.1656 | 0.99974 | 0.99975 | 1 | 17655 | 0 | −1.1656 |
| MRPS24 | 4 | 0.00026303 | 0.00096388 | 0.033145 | 524 | 3 | −2.2821 | 0.94336 | 0.94321 | 1 | 15330 | 0 | −2.2821 |
| SMG7 | 4 | 0.00026547 | 0.0009754 | 0.033477 | 525 | 3 | −1.4299 | 0.82406 | 0.84168 | 1 | 13412 | 1 | −1.4299 |
| COX5B | 4 | 0.00026981 | 0.00098856 | 0.033793 | 526 | 4 | −1.7509 | 0.99973 | 0.99974 | 1 | 17654 | 0 | −1.7509 |
| TRNT1 | 4 | 0.00027008 | 0.00099021 | 0.033793 | 527 | 2 | −1.4256 | 0.29249 | 0.47873 | 1 | 8279 | 2 | −1.4256 |
| C7orf55-LUC7L2 | 4 | 0.00027011 | 0.00099021 | 0.033793 | 528 | 4 | −1.3135 | 0.99542 | 0.99551 | 1 | 17088 | 0 | −1.3135 |
| GARS | 4 | 0.00027204 | 0.0010006 | 0.034084 | 529 | 3 | −2.6454 | 0.76824 | 0.81033 | 1 | 12851 | 1 | −2.6454 |
| SLC25A19 | 4 | 0.00027315 | 0.0010034 | 0.034113 | 530 | 3 | −1.2854 | 0.16008 | 0.32012 | 0.935009 | 6112 | 1 | −1.2854 |
| RNF40 | 4 | 0.00027423 | 0.0010056 | 0.034123 | 531 | 4 | −2.0252 | 0.99973 | 0.99973 | 1 | 17653 | 0 | −2.0252 |
| WDR82 | 4 | 0.00027508 | 0.0010089 | 0.03417 | 532 | 4 | −1.3158 | 0.99972 | 0.99973 | 1 | 17652 | 0 | −1.3158 |
| UQCRFS1 | 4 | 0.00028152 | 0.0010291 | 0.034793 | 533 | 4 | −1.9863 | 0.99972 | 0.99973 | 1 | 17651 | 0 | −1.9863 |
| WDR12 | 4 | 0.00028312 | 0.0010385 | 0.035042 | 534 | 4 | −1.2201 | 0.99972 | 0.99973 | 1 | 17650 | 0 | −1.2201 |
| NDUFA10 | 4 | 0.00028374 | 0.0010423 | 0.035106 | 535 | 4 | −1.2124 | 0.99972 | 0.99973 | 1 | 17649 | 0 | −1.2124 |
| NAT10 | 4 | 0.00028783 | 0.0010555 | 0.035483 | 536 | 4 | −1.155 | 0.99971 | 0.99972 | 1 | 17646 | 0 | −1.155 |
| MST1 | 4 | 0.00028946 | 0.0010593 | 0.035517 | 537 | 4 | −1.3002 | 0.99971 | 0.99972 | 1 | 17645 | 0 | −1.3002 |
| LAS1L | 4 | 0.00029134 | 0.0010653 | 0.035616 | 538 | 4 | −1.8234 | 0.99971 | 0.99972 | 1 | 17644 | 0 | −1.8234 |
| FAM210A | 4 | 0.00029445 | 0.0010785 | 0.03596 | 539 | 4 | −0.94879 | 0.99401 | 0.99412 | 1 | 16996 | 0 | −0.94879 |
| EIF5 | 4 | 0.00029476 | 0.0010796 | 0.03596 | 540 | 4 | −0.94043 | 0.99971 | 0.99972 | 1 | 17643 | 0 | −0.94043 |
| MRPL14 | 4 | 0.00029718 | 0.0010856 | 0.036094 | 541 | 4 | −2.8913 | 0.9997 | 0.99971 | 1 | 17642 | 0 | −2.8913 |
| IARS | 4 | 0.00030167 | 0.0010993 | 0.036483 | 542 | 4 | −1.5842 | 0.9997 | 0.99971 | 1 | 17640 | 0 | −1.5842 |
| PABPC1L | 4 | 0.00030219 | 0.0011026 | 0.036512 | 543 | 4 | −0.92202 | 0.9997 | 0.99971 | 1 | 17639 | 0 | −0.92202 |
| TKT | 4 | 0.00030297 | 0.0011043 | 0.036512 | 544 | 4 | −1.0769 | 0.9997 | 0.99971 | 1 | 17638 | 0 | −1.0769 |
| INTS3 | 4 | 0.00030701 | 0.0011131 | 0.036735 | 545 | 4 | −0.86475 | 0.99969 | 0.99971 | 1 | 17637 | 0 | −0.86475 |
| EXOSC9 | 4 | 0.00030804 | 0.0011196 | 0.036836 | 546 | 4 | −2.2993 | 0.98585 | 0.98591 | 1 | 16587 | 0 | −2.2993 |
| NCAPD2 | 4 | 0.00030845 | 0.0011202 | 0.036836 | 547 | 4 | −1.0423 | 0.99969 | 0.99971 | 1 | 17636 | 0 | −1.0423 |
| CCDC94 | 4 | 0.00031069 | 0.0011279 | 0.037007 | 548 | 4 | −1.1893 | 0.99969 | 0.9997 | 1 | 17635 | 0 | −1.1893 |
| PEAR1 | 4 | 0.00031135 | 0.0011295 | 0.037007 | 549 | 4 | −0.87898 | 0.99969 | 0.9997 | 1 | 17634 | 0 | −0.87898 |
| HDAC3 | 4 | 0.00031294 | 0.0011366 | 0.037174 | 550 | 4 | −1.2249 | 0.99969 | 0.9997 | 1 | 17633 | 0 | −1.2249 |
| YAE1D1 | 4 | 0.00031743 | 0.0011525 | 0.037626 | 551 | 4 | −1.4442 | 0.99964 | 0.99966 | 1 | 17623 | 0 | −1.4442 |
| MRPL17 | 4 | 0.00031927 | 0.0011608 | 0.037826 | 552 | 4 | −1.3478 | 0.98932 | 0.98944 | 1 | 16738 | 0 | −1.3478 |
| TCOF1 | 4 | 0.00032763 | 0.0011838 | 0.038507 | 553 | 4 | −1.0286 | 0.99564 | 0.99572 | 1 | 17102 | 0 | −1.0286 |
| MYCT1 | 4 | 0.0003296 | 0.0011909 | 0.038669 | 554 | 4 | −0.98271 | 0.99967 | 0.99969 | 1 | 17632 | 0 | −0.98271 |
| DDX28 | 4 | 0.00033045 | 0.0011931 | 0.038671 | 555 | 3 | −1.6844 | 0.64213 | 0.75602 | 1 | 11995 | 1 | −1.6844 |
| MYBBP1A | 4 | 0.00033303 | 0.0012014 | 0.038868 | 556 | 3 | −1.5238 | 0.91341 | 0.91319 | 1 | 14690 | 0 | −1.5238 |
| MASTL | 4 | 0.00033741 | 0.0012184 | 0.039277 | 557 | 3 | −2.34 | 0.42505 | 0.58991 | 1 | 9734 | 1 | −2.34 |
| NAMPT | 4 | 0.00033825 | 0.0012211 | 0.039295 | 558 | 4 | −0.73348 | 0.99966 | 0.99968 | 1 | 17631 | 0 | −0.73348 |
| DPM1 | 4 | 0.00033966 | 0.0012271 | 0.039419 | 559 | 4 | −1.0231 | 0.99966 | 0.99968 | 1 | 17630 | 0 | −1.0231 |
| GTPBP8 | 4 | 0.00034164 | 0.001231 | 0.039472 | 560 | 4 | −1.6698 | 0.99966 | 0.99968 | 1 | 17628 | 0 | −1.6698 |
| SSBP1 | 4 | 0.00034285 | 0.0012376 | 0.039613 | 561 | 3 | −0.99483 | 0.91602 | 0.91583 | 1 | 14737 | 0 | −0.99483 |
| AHCY | 4 | 0.00034382 | 0.001243 | 0.039683 | 562 | 4 | −1.673 | 0.99057 | 0.99065 | 1 | 16805 | 0 | −1.673 |
| CIAO1 | 4 | 0.00034435 | 0.0012441 | 0.039683 | 563 | 4 | −1.5027 | 0.99966 | 0.99967 | 1 | 17627 | 0 | −1.5027 |
| ACTR6 | 4 | 0.00034608 | 0.0012491 | 0.03977 | 564 | 3 | −1.6419 | 0.8031 | 0.8292 | 1 | 13154 | 1 | −1.6419 |
| SZT2 | 4 | 0.00034894 | 0.0012611 | 0.040065 | 565 | 4 | −0.99545 | 0.99965 | 0.99967 | 1 | 17625 | 0 | −0.99545 |
| UQCRC2 | 4 | 0.00034998 | 0.0012628 | 0.040065 | 566 | 3 | −2.5918 | 0.37781 | 0.55002 | 1 | 9200 | 1 | −2.5918 |
| LETM1 | 3 | 0.00035135 | 0.0010604 | 0.035517 | 567 | 3 | −1.1944 | 0.99965 | 0.99964 | 1 | 17624 | 0 | −1.1944 |
| MOGS | 4 | 0.00036194 | 0.0013072 | 0.041329 | 568 | 4 | −0.76983 | 0.99964 | 0.99965 | 1 | 17622 | 0 | −0.76983 |
| GPN2 | 4 | 0.00036283 | 0.0013138 | 0.041444 | 569 | 4 | −2.2523 | 0.99964 | 0.99965 | 1 | 17621 | 0 | −2.2523 |
| EIF4E | 4 | 0.00036319 | 0.0013154 | 0.041444 | 570 | 4 | −0.94508 | 0.99928 | 0.99928 | 1 | 17507 | 0 | −0.94508 |
| CCT5 | 4 | 0.00036432 | 0.001322 | 0.041579 | 571 | 4 | −1.872 | 0.99964 | 0.99965 | 1 | 17620 | 0 | −1.872 |
| DNAJC9 | 4 | 0.00036491 | 0.001327 | 0.041662 | 572 | 4 | −1.1976 | 0.99964 | 0.99965 | 1 | 17619 | 0 | −1.1976 |
| PRMT5 | 4 | 0.00036687 | 0.0013357 | 0.041864 | 573 | 4 | −2.4543 | 0.99763 | 0.99765 | 1 | 17276 | 0 | −2.4543 |
| ATXN7L2 | 4 | 0.00037227 | 0.0013538 | 0.042353 | 574 | 4 | −0.66798 | 0.99963 | 0.99964 | 1 | 17618 | 0 | −0.66798 |
| TBL3 | 4 | 0.0003733 | 0.001356 | 0.042353 | 575 | 4 | −1.2264 | 0.98883 | 0.98896 | 1 | 16713 | 0 | −1.2264 |
| EIF5A | 2 | 0.00037362 | 0.00071215 | 0.027009 | 576 | 2 | −2.0726 | 0.99963 | 0.99961 | 1 | 17616 | 0 | −2.0726 |
| CCDC86 | 4 | 0.0003776 | 0.001367 | 0.042622 | 577 | 4 | −1.3858 | 0.99962 | 0.99963 | 1 | 17615 | 0 | −1.3858 |
| COQ7 | 4 | 0.00038188 | 0.001384 | 0.043004 | 578 | 4 | −0.9574 | 0.99722 | 0.99725 | 1 | 17225 | 0 | −0.9574 |
| TUBD1 | 4 | 0.00038222 | 0.001384 | 0.043004 | 579 | 4 | −1.3197 | 0.99949 | 0.99951 | 1 | 17563 | 0 | −1.3197 |
| TOMM70A | 4 | 0.00038351 | 0.0013873 | 0.043032 | 580 | 4 | −1.7198 | 0.99728 | 0.99731 | 1 | 17234 | 0 | −1.7198 |
| SEMA4A | 4 | 0.00038485 | 0.0013911 | 0.043077 | 581 | 4 | −1.0967 | 0.99828 | 0.99831 | 1 | 17347 | 0 | −1.0967 |
| DNM2 | 4 | 0.00038639 | 0.0013966 | 0.043116 | 582 | 4 | −1.6188 | 0.99961 | 0.99963 | 1 | 17614 | 0 | −1.6188 |
| UQCC2 | 4 | 0.00038655 | 0.0013972 | 0.043116 | 583 | 4 | −1.0511 | 0.99961 | 0.99963 | 1 | 17613 | 0 | −1.0511 |
| NOP56 | 4 | 0.00038998 | 0.0014114 | 0.043449 | 584 | 4 | −1.1803 | 0.99961 | 0.99962 | 1 | 17612 | 0 | −1.1803 |
| ATP6V1D | 4 | 0.00039045 | 0.0014131 | 0.043449 | 585 | 4 | −1.8622 | 0.99961 | 0.99962 | 1 | 17611 | 0 | −1.8622 |
| MRPL54 | 4 | 0.00039155 | 0.0014163 | 0.043449 | 586 | 4 | −1.2266 | 0.99961 | 0.99962 | 1 | 17610 | 0 | −1.2266 |
| ORAOV1 | 4 | 0.00039249 | 0.0014202 | 0.043449 | 587 | 4 | −1.5957 | 0.99961 | 0.99962 | 1 | 17609 | 0 | −1.5957 |
| RNF168 | 4 | 0.00039268 | 0.0014213 | 0.043449 | 588 | 4 | −1.4451 | 0.98168 | 0.98176 | 1 | 16420 | 0 | −1.4451 |
| NLE1 | 4 | 0.00039328 | 0.0014224 | 0.043449 | 589 | 4 | −1.7125 | 0.99961 | 0.99962 | 1 | 17608 | 0 | −1.7125 |
| TUBG1 | 4 | 0.00039533 | 0.0014301 | 0.043496 | 590 | 4 | −0.94529 | 0.9996 | 0.99962 | 1 | 17607 | 0 | −0.94529 |
| FBXL16 | 4 | 0.00039549 | 0.0014312 | 0.043496 | 591 | 4 | −0.52202 | 0.9996 | 0.99962 | 1 | 17606 | 0 | −0.52202 |
| FLYWCH1 | 4 | 0.00039551 | 0.0014312 | 0.043496 | 592 | 4 | −0.9228 | 0.99877 | 0.99876 | 1 | 17418 | 0 | −0.9228 |
| PKM | 4 | 0.00039728 | 0.001435 | 0.043533 | 593 | 3 | −1.2716 | 0.7263 | 0.79018 | 1 | 12527 | 1 | −1.2716 |
| C11orf57 | 4 | 0.00039834 | 0.0014372 | 0.043533 | 594 | 3 | −1.1236 | 0.95904 | 0.95892 | 1 | 15721 | 0 | −1.1236 |
| SLC3A1 | 4 | 0.00039947 | 0.0014421 | 0.043569 | 595 | 4 | −0.92914 | 0.9996 | 0.99962 | 1 | 17605 | 0 | −0.92914 |
| WDR46 | 4 | 0.00039976 | 0.0014432 | 0.043569 | 596 | 4 | −1.5042 | 0.99519 | 0.99527 | 1 | 17071 | 0 | −1.5042 |
| CCNL1 | 4 | 0.00040138 | 0.0014482 | 0.043645 | 597 | 4 | −0.88924 | 0.9996 | 0.99962 | 1 | 17604 | 0 | −0.88924 |
| NARS2 | 4 | 0.00040369 | 0.0014553 | 0.043787 | 598 | 3 | −1.9586 | 0.69184 | 0.77534 | 1 | 12303 | 1 | −1.9586 |
| RAP1A | 4 | 0.00040556 | 0.0014668 | 0.04406 | 599 | 4 | −0.90525 | 0.99959 | 0.99961 | 1 | 17603 | 0 | −0.90525 |
| EEF1A1 | 3 | 0.00040558 | 0.0012079 | 0.039011 | 600 | 3 | −2.7802 | 0.99959 | 0.99959 | 1 | 17602 | 0 | −2.7802 |
| CDC37 | 4 | 0.00040662 | 0.0014717 | 0.04412 | 601 | 4 | −1.7143 | 0.9953 | 0.99539 | 1 | 17077 | 0 | −1.7143 |
| NOL11 | 4 | 0.00040693 | 0.0014739 | 0.04412 | 602 | 4 | −1.3156 | 0.99954 | 0.99957 | 1 | 17588 | 0 | −1.3156 |
| MRPL41 | 4 | 0.00040799 | 0.0014761 | 0.04412 | 603 | 4 | −1.0938 | 0.99959 | 0.99961 | 1 | 17601 | 0 | −1.0938 |
| VCP | 4 | 0.00042045 | 0.0015173 | 0.045275 | 604 | 4 | −1.2584 | 0.99958 | 0.9996 | 1 | 17600 | 0 | −1.2584 |
| THG1L | 4 | 0.00042177 | 0.0015227 | 0.045344 | 605 | 4 | −1.9499 | 0.99958 | 0.9996 | 1 | 17599 | 0 | −1.9499 |
| TTC27 | 4 | 0.00042409 | 0.0015255 | 0.045344 | 606 | 3 | −1.926 | 0.79498 | 0.82459 | 1 | 13087 | 1 | −1.926 |
| EIF2B3 | 4 | 0.00042483 | 0.0015271 | 0.045344 | 607 | 4 | −0.99833 | 0.99195 | 0.99207 | 1 | 16881 | 0 | −0.99833 |
| SLC7A6OS | 4 | 0.00042594 | 0.0015299 | 0.045351 | 608 | 4 | −1.0616 | 0.99957 | 0.99959 | 1 | 17598 | 0 | −1.0616 |
| RAD51D | 4 | 0.00042757 | 0.0015376 | 0.045504 | 609 | 3 | −1.7036 | 0.82468 | 0.84203 | 1 | 13420 | 1 | −1.7036 |
| HAUS1 | 3 | 0.00042826 | 0.0012853 | 0.040707 | 610 | 3 | −1.5579 | 0.99957 | 0.99956 | 1 | 17596 | 0 | −1.5579 |
| TSEN34 | 4 | 0.00042898 | 0.0015419 | 0.045559 | 611 | 3 | −1.1299 | 0.3665 | 0.54057 | 1 | 9073 | 1 | −1.1299 |
| RUSC1 | 4 | 0.00043268 | 0.0015524 | 0.045792 | 612 | 4 | −1.0341 | 0.99957 | 0.99959 | 1 | 17595 | 0 | −1.0341 |
| ZNRD1 | 4 | 0.00043466 | 0.0015589 | 0.045911 | 613 | 4 | −1.1365 | 0.96358 | 0.96348 | 1 | 15833 | 0 | −1.1365 |
| NDUFA6 | 4 | 0.00043755 | 0.0015688 | 0.046127 | 614 | 3 | −1.3278 | 0.077976 | 0.18649 | 0.808703 | 4111 | 1 | −1.3278 |
| WDR24 | 4 | 0.00044155 | 0.0015864 | 0.046567 | 615 | 4 | −0.85162 | 0.99956 | 0.99958 | 1 | 17594 | 0 | −0.85162 |
| IPO11 | 4 | 0.00044401 | 0.001594 | 0.046716 | 616 | 4 | −1.4959 | 0.99835 | 0.99837 | 1 | 17356 | 0 | −1.4959 |
| KARS | 4 | 0.00044466 | 0.0015968 | 0.046721 | 617 | 4 | −2.1744 | 0.99956 | 0.99958 | 1 | 17593 | 0 | −2.1744 |
| MRPL46 | 4 | 0.00044639 | 0.0016045 | 0.046869 | 618 | 4 | −1.2807 | 0.99955 | 0.99957 | 1 | 17592 | 0 | −1.2807 |
| CDK4 | 4 | 0.00044743 | 0.0016088 | 0.046922 | 619 | 4 | −0.85643 | 0.99955 | 0.99957 | 1 | 17591 | 0 | −0.85643 |
| NDUFB9 | 4 | 0.00044812 | 0.0016116 | 0.046926 | 620 | 4 | −1.8892 | 0.99955 | 0.99957 | 1 | 17590 | 0 | −1.8892 |
| TSR1 | 4 | 0.0004486 | 0.0016154 | 0.046962 | 621 | 4 | −1.6715 | 0.99397 | 0.99407 | 1 | 16992 | 0 | −1.6715 |
| COMMD4 | 4 | 0.00045144 | 0.0016291 | 0.047228 | 622 | 4 | −0.88776 | 0.99955 | 0.99957 | 1 | 17589 | 0 | −0.88776 |
| PSTK | 4 | 0.00045168 | 0.0016308 | 0.047228 | 623 | 3 | −1.6669 | 0.93861 | 0.93847 | 1 | 15214 | 0 | −1.6669 |
| ERCC4 | 4 | 0.00045245 | 0.0016324 | 0.047228 | 624 | 3 | −1.1777 | 0.93868 | 0.93854 | 1 | 15217 | 0 | −1.1777 |
| SMARCA5 | 4 | 0.00045555 | 0.0016423 | 0.047438 | 625 | 3 | −2.1214 | 0.95842 | 0.95834 | 1 | 15706 | 0 | −2.1214 |
| TMEM242 | 4 | 0.00045983 | 0.0016582 | 0.047716 | 626 | 3 | −1.3673 | 0.92059 | 0.92037 | 1 | 14834 | 0 | −1.3673 |
| TPT1 | 4 | 0.00046042 | 0.0016588 | 0.047716 | 627 | 4 | −1.2805 | 0.99954 | 0.99956 | 1 | 17587 | 0 | −1.2805 |
| POLR1D | 4 | 0.00046061 | 0.0016599 | 0.047716 | 628 | 3 | −1.2978 | 0.94826 | 0.94818 | 1 | 15437 | 0 | −1.2978 |
| SCAND1 | 4 | 0.00046238 | 0.0016648 | 0.047781 | 629 | 4 | −1.3412 | 0.99954 | 0.99956 | 1 | 17586 | 0 | −1.3412 |
| CUL1 | 4 | 0.00046492 | 0.0016769 | 0.048051 | 630 | 4 | −1.1215 | 0.96974 | 0.96965 | 1 | 16020 | 0 | −1.1215 |
| TCFL5 | 4 | 0.00046864 | 0.0016955 | 0.048509 | 631 | 4 | −0.81591 | 0.99953 | 0.99955 | 1 | 17584 | 0 | −0.81591 |
| NOL10 | 4 | 0.00047045 | 0.001701 | 0.048588 | 632 | 4 | −0.99082 | 0.98885 | 0.98898 | 1 | 16715 | 0 | −0.99082 |
| UPF2 | 4 | 0.00047315 | 0.0017098 | 0.048732 | 633 | 4 | −1.0379 | 0.99953 | 0.99955 | 1 | 17583 | 0 | −1.0379 |
| RPE | 4 | 0.00047369 | 0.0017114 | 0.048732 | 634 | 4 | −1.4637 | 0.99953 | 0.99955 | 1 | 17582 | 0 | −1.4637 |
| BCAS2 | 4 | 0.00047514 | 0.001718 | 0.048842 | 635 | 4 | −0.89516 | 0.99952 | 0.99955 | 1 | 17580 | 0 | −0.89516 |
| SUGT1 | 4 | 0.00047934 | 0.0017333 | 0.049078 | 636 | 4 | −1.6713 | 0.99952 | 0.99954 | 1 | 17579 | 0 | −1.6713 |
| WDR18 | 4 | 0.00047979 | 0.0017339 | 0.049078 | 637 | 3 | −1.9455 | 0.54677 | 0.69346 | 1 | 11108 | 1 | −1.9455 |
| BOP1 | 4 | 0.00048025 | 0.0017344 | 0.049078 | 638 | 4 | −1.5551 | 0.99952 | 0.99954 | 1 | 17578 | 0 | −1.5551 |
| TIMM44 | 4 | 0.00048337 | 0.0017449 | 0.049296 | 639 | 4 | −0.87568 | 0.99952 | 0.99954 | 1 | 17577 | 0 | −0.87568 |
| PHB2 | 4 | 0.00048577 | 0.0017525 | 0.049343 | 640 | 4 | −2.9838 | 0.99951 | 0.99954 | 1 | 17576 | 0 | −2.9838 |
| IMPDH2 | 4 | 0.00048633 | 0.0017531 | 0.049343 | 641 | 4 | −1.0734 | 0.99951 | 0.99954 | 1 | 17575 | 0 | −1.0734 |
| HIPK1 | 4 | 0.00048725 | 0.0017547 | 0.049343 | 642 | 4 | −0.78711 | 0.99951 | 0.99953 | 1 | 17574 | 0 | −0.78711 |
| MRPL11 | 4 | 0.00048891 | 0.0017608 | 0.04942 | 643 | 3 | −1.5494 | 0.44738 | 0.60874 | 1 | 9983 | 1 | −1.5494 |
| FOXM1 | 4 | 0.00048929 | 0.001763 | 0.04942 | 644 | 4 | −1.1859 | 0.99951 | 0.99953 | 1 | 17573 | 0 | −1.1859 |
| SNAPC3 | 4 | 0.00048985 | 0.0017663 | 0.049421 | 645 | 4 | −1.3538 | 0.99951 | 0.99953 | 1 | 17572 | 0 | −1.3538 |
| SMC4 | 4 | 0.00049041 | 0.0017684 | 0.049421 | 646 | 4 | −1.045 | 0.99951 | 0.99953 | 1 | 17571 | 0 | −1.045 |
| CDAN1 | 4 | 0.00049503 | 0.0017854 | 0.049819 | 647 | 4 | −1.26 | 0.98627 | 0.98633 | 1 | 16601 | 0 | −1.26 |
| NIP7 | 4 | 0.00049639 | 0.0017909 | 0.049848 | 648 | 4 | −1.2897 | 0.9995 | 0.99952 | 1 | 17570 | 0 | −1.2897 |
| PIAS4 | 4 | 0.00049676 | 0.001792 | 0.049848 | 649 | 4 | −0.67954 | 0.9995 | 0.99952 | 1 | 17569 | 0 | −0.67954 |
| CEP85 | 4 | 0.00049695 | 0.0017948 | 0.049848 | 650 | 4 | −0.97137 | 0.9995 | 0.99952 | 1 | 17568 | 0 | −0.97137 |
| WDR83 | 4 | 0.00050147 | 0.0018101 | 0.050197 | 651 | 4 | −1.0869 | 0.9995 | 0.99952 | 1 | 17567 | 0 | −1.0869 |
| NDUFAF5 | 4 | 0.00050573 | 0.001826 | 0.05056 | 652 | 3 | −1.4331 | 0.71489 | 0.78504 | 1 | 12445 | 1 | −1.4331 |
| FAM212B | 4 | 0.00051004 | 0.0018359 | 0.050756 | 653 | 4 | −0.81748 | 0.99949 | 0.99951 | 1 | 17565 | 0 | −0.81748 |
| TRIM46 | 4 | 0.00051158 | 0.0018436 | 0.05089 | 654 | 4 | −1.1143 | 0.99949 | 0.99951 | 1 | 17564 | 0 | −1.1143 |
| SETD1A | 4 | 0.00052045 | 0.0018787 | 0.051679 | 655 | 4 | −1.6935 | 0.97309 | 0.97305 | 1 | 16128 | 0 | −1.6935 |
| ISG20L2 | 4 | 0.00052066 | 0.0018792 | 0.051679 | 656 | 4 | −1.2203 | 0.99948 | 0.9995 | 1 | 17561 | 0 | −1.2203 |
| EIF1AD | 4 | 0.00052222 | 0.0018825 | 0.051679 | 657 | 4 | −1.1649 | 0.99948 | 0.9995 | 1 | 17560 | 0 | −1.1649 |
| DCTN3 | 4 | 0.00052299 | 0.0018836 | 0.051679 | 658 | 4 | −1.4649 | 0.97953 | 0.97963 | 1 | 16342 | 0 | −1.4649 |
| MRPS18B | 4 | 0.00052342 | 0.0018869 | 0.051691 | 659 | 4 | −1.7458 | 0.99331 | 0.99342 | 1 | 16952 | 0 | −1.7458 |
| RPS21 | 4 | 0.00052888 | 0.0019072 | 0.052168 | 660 | 4 | −1.4714 | 0.99947 | 0.99949 | 1 | 17559 | 0 | −1.4714 |
| MRPS21 | 4 | 0.00053283 | 0.0019187 | 0.052382 | 661 | 4 | −1.2227 | 0.99947 | 0.99949 | 1 | 17558 | 0 | −1.2227 |
| SHQ1 | 4 | 0.00053498 | 0.0019253 | 0.052382 | 662 | 4 | −1.1266 | 0.9883 | 0.98839 | 1 | 16691 | 0 | −1.1266 |
| MORN1 | 4 | 0.00053521 | 0.0019258 | 0.052382 | 663 | 4 | −0.81309 | 0.99946 | 0.99948 | 1 | 17557 | 0 | −0.81309 |
| NOP10 | 4 | 0.00053623 | 0.0019297 | 0.052382 | 664 | 3 | −0.66534 | 0.14738 | 0.30041 | 0.921099 | 5817 | 1 | −0.66534 |
| DCTN2 | 4 | 0.0005368 | 0.0019308 | 0.052382 | 665 | 4 | −1.3012 | 0.99946 | 0.99948 | 1 | 17556 | 0 | −1.3012 |
| FAM83C | 4 | 0.000538 | 0.0019324 | 0.052382 | 666 | 4 | −1.2438 | 0.99946 | 0.99948 | 1 | 17555 | 0 | −1.2438 |
| ASCC3 | 4 | 0.00054017 | 0.0019385 | 0.052466 | 667 | 3 | −1.0845 | 0.48653 | 0.64191 | 1 | 10378 | 1 | −1.0845 |
| NAA35 | 4 | 0.00054496 | 0.0019604 | 0.052931 | 668 | 3 | −1.9519 | 0.9146 | 0.91439 | 1 | 14715 | 0 | −1.9519 |
| COMMD10 | 4 | 0.00054501 | 0.0019615 | 0.052931 | 669 | 4 | −0.50709 | 0.99945 | 0.99947 | 1 | 17554 | 0 | −0.50709 |
| MMP23B | 4 | 0.00054845 | 0.0019736 | 0.053177 | 670 | 4 | −0.86049 | 0.99945 | 0.99947 | 1 | 17553 | 0 | −0.86049 |
| PDCD11 | 4 | 0.00054963 | 0.001979 | 0.053245 | 671 | 4 | −1.2264 | 0.99739 | 0.99742 | 1 | 17246 | 0 | −1.2264 |
| ARHGAP30 | 4 | 0.00055067 | 0.0019851 | 0.053328 | 672 | 4 | −0.96891 | 0.99945 | 0.99947 | 1 | 17552 | 0 | −0.96891 |
| HNRNPL | 4 | 0.00055373 | 0.0019955 | 0.053529 | 673 | 4 | −1.3456 | 0.99945 | 0.99946 | 1 | 17551 | 0 | −1.3456 |
| TEAD4 | 4 | 0.00055495 | 0.0019999 | 0.053567 | 674 | 4 | −0.76489 | 0.99945 | 0.99946 | 1 | 17550 | 0 | −0.76489 |
| KPNA4 | 4 | 0.00055618 | 0.0020081 | 0.053707 | 675 | 4 | −0.98362 | 0.99944 | 0.99946 | 1 | 17549 | 0 | −0.98362 |
| HNRNPU | 4 | 0.00056038 | 0.0020273 | 0.054141 | 676 | 4 | −2.6256 | 0.99746 | 0.99749 | 1 | 17252 | 0 | −2.6256 |
| ACTR1B | 4 | 0.00056854 | 0.0020657 | 0.055084 | 677 | 4 | −1.0338 | 0.99943 | 0.99945 | 1 | 17548 | 0 | −1.0338 |
| PDCD2 | 4 | 0.00057292 | 0.0020772 | 0.055257 | 678 | 4 | −1.1175 | 0.99943 | 0.99945 | 1 | 17545 | 0 | −1.1175 |
| MPI | 4 | 0.00057355 | 0.0020783 | 0.055257 | 679 | 4 | −0.562 | 0.99943 | 0.99945 | 1 | 17544 | 0 | −0.562 |
| IYD | 4 | 0.00057649 | 0.0020904 | 0.055497 | 680 | 4 | −0.82784 | 0.99942 | 0.99944 | 1 | 17543 | 0 | −0.82784 |
| MAP2K2 | 4 | 0.00058016 | 0.0021057 | 0.055822 | 681 | 4 | −1.0697 | 0.97703 | 0.9771 | 1 | 16264 | 0 | −1.0697 |
| CTDNEP1 | 4 | 0.00058302 | 0.0021151 | 0.055987 | 682 | 4 | −1.8561 | 0.99942 | 0.99944 | 1 | 17542 | 0 | −1.8561 |
| ITFG2 | 4 | 0.0005861 | 0.0021244 | 0.056152 | 683 | 4 | −1.1657 | 0.99699 | 0.99703 | 1 | 17198 | 0 | −1.1657 |
| NUDCD3 | 4 | 0.00058876 | 0.0021326 | 0.056287 | 684 | 4 | −1.8125 | 0.99941 | 0.99943 | 1 | 17541 | 0 | −1.8125 |
| TSR2 | 4 | 0.00059133 | 0.0021408 | 0.056421 | 685 | 4 | −1.2899 | 0.99941 | 0.99943 | 1 | 17539 | 0 | −1.2899 |
| TOMM34 | 4 | 0.00059864 | 0.0021694 | 0.057006 | 686 | 4 | −0.9283 | 0.9994 | 0.99942 | 1 | 17538 | 0 | −0.9283 |
| ELP3 | 4 | 0.00059867 | 0.0021694 | 0.057006 | 687 | 4 | −1.0916 | 0.99009 | 0.99018 | 1 | 16778 | 0 | −1.0916 |
| EMC4 | 4 | 0.00060087 | 0.0021765 | 0.057111 | 688 | 4 | −1.3631 | 0.9891 | 0.98923 | 1 | 16727 | 0 | −1.3631 |
| ADRM1 | 4 | 0.00060297 | 0.0021864 | 0.057286 | 689 | 4 | −1.004 | 0.9994 | 0.99942 | 1 | 17536 | 0 | −1.004 |
| PHF23 | 4 | 0.00061018 | 0.0022116 | 0.05778 | 690 | 4 | −0.63932 | 0.99939 | 0.99941 | 1 | 17535 | 0 | −0.63932 |
| CHTF18 | 4 | 0.00061023 | 0.0022116 | 0.05778 | 691 | 3 | −0.8006 | 0.36091 | 0.53588 | 1 | 9016 | 1 | −0.8006 |
| CCT6A | 4 | 0.00061325 | 0.0022275 | 0.058042 | 692 | 4 | −2.1155 | 0.99939 | 0.9994 | 1 | 17534 | 0 | −2.1155 |
| WDR92 | 4 | 0.00061347 | 0.002228 | 0.058042 | 693 | 4 | −1.0298 | 0.99939 | 0.9994 | 1 | 17533 | 0 | −1.0298 |
| GAL3ST1 | 4 | 0.00061943 | 0.0022461 | 0.058429 | 694 | 4 | −0.57027 | 0.99938 | 0.9994 | 1 | 17532 | 0 | −0.57027 |
| VAV3 | 4 | 0.00062611 | 0.0022703 | 0.05889 | 695 | 4 | −0.89096 | 0.99937 | 0.99939 | 1 | 17531 | 0 | −0.89096 |
| DDX27 | 4 | 0.00062768 | 0.0022741 | 0.05889 | 696 | 4 | −1.1739 | 0.99937 | 0.99939 | 1 | 17530 | 0 | −1.1739 |
| TRIM45 | 4 | 0.00062924 | 0.0022774 | 0.05889 | 697 | 4 | −0.83014 | 0.99937 | 0.99939 | 1 | 17529 | 0 | −0.83014 |
| CELF1 | 4 | 0.00063037 | 0.0022818 | 0.05889 | 698 | 4 | −0.54422 | 0.99937 | 0.99939 | 1 | 17528 | 0 | −0.54422 |
| PNKP | 4 | 0.00063067 | 0.0022834 | 0.05889 | 699 | 3 | −1.5824 | 0.88787 | 0.88862 | 1 | 14248 | 1 | −1.5824 |
| MRPS26 | 4 | 0.00063115 | 0.0022845 | 0.05889 | 700 | 3 | −2.1486 | 0.46751 | 0.62583 | 1 | 10158 | 1 | −2.1486 |
| KCNC4 | 4 | 0.00063172 | 0.0022867 | 0.05889 | 701 | 4 | −0.9695 | 0.99937 | 0.99939 | 1 | 17527 | 0 | −0.9695 |
| CCDC101 | 4 | 0.00063419 | 0.0022982 | 0.059103 | 702 | 4 | −0.83065 | 0.99937 | 0.99938 | 1 | 17526 | 0 | −0.83065 |
| MTX1 | 4 | 0.00063555 | 0.0023032 | 0.059145 | 703 | 4 | −1.0054 | 0.99936 | 0.99938 | 1 | 17525 | 0 | −1.0054 |
| UROD | 4 | 0.00063742 | 0.0023103 | 0.059244 | 704 | 4 | −0.99587 | 0.99017 | 0.99025 | 1 | 16782 | 0 | −0.99587 |
| PMPCA | 4 | 0.0006394 | 0.0023191 | 0.059343 | 705 | 4 | −1.5048 | 0.99936 | 0.99938 | 1 | 17523 | 0 | −1.5048 |
| DRAP1 | 4 | 0.00063984 | 0.0023207 | 0.059343 | 706 | 3 | −1.2807 | 0.59767 | 0.73762 | 1 | 11739 | 1 | −1.2807 |
| MRPS31 | 4 | 0.00064519 | 0.0023399 | 0.059749 | 707 | 4 | −1.3588 | 0.99905 | 0.99904 | 1 | 17470 | 0 | −1.3588 |
| PALB2 | 4 | 0.00065008 | 0.0023569 | 0.060098 | 708 | 4 | −2.1732 | 0.99931 | 0.99932 | 1 | 17515 | 0 | −2.1732 |
| TXN2 | 4 | 0.00065943 | 0.0023948 | 0.060827 | 709 | 3 | −1.8973 | 0.58489 | 0.72639 | 1 | 11573 | 1 | −1.8973 |
| GID8 | 4 | 0.00065984 | 0.002397 | 0.060827 | 710 | 4 | −1.2571 | 0.99934 | 0.99936 | 1 | 17521 | 0 | −1.2571 |
| PWP2 | 4 | 0.00066042 | 0.0024008 | 0.060827 | 711 | 4 | −1.6884 | 0.9949 | 0.99058 | 1 | 16801 | 0 | −1.6884 |
| SYT11 | 4 | 0.00066054 | 0.0024013 | 0.060827 | 712 | 4 | −0.90791 | 0.99934 | 0.99936 | 1 | 17520 | 0 | −0.90791 |
| TEFM | 4 | 0.00066171 | 0.0024052 | 0.060827 | 713 | 4 | −1.6895 | 0.99934 | 0.99935 | 1 | 17519 | 0 | −1.6895 |
| GEMIN4 | 4 | 0.000662 | 0.0024057 | 0.060827 | 714 | 3 | −1.0834 | 0.92934 | 0.92907 | 1 | 15010 | 0 | −1.0834 |
| PGK1 | 4 | 0.00066639 | 0.0024238 | 0.061141 | 715 | 3 | −2.2262 | 0.79625 | 0.82529 | 1 | 13098 | 1 | −2.2262 |
| ZBTB8OS | 4 | 0.00066688 | 0.0024249 | 0.061141 | 716 | 3 | −1.3488 | 0.83652 | 0.84965 | 1 | 13557 | 1 | −1.3488 |
| SYPL2 | 4 | 0.00067107 | 0.0024397 | 0.061429 | 717 | 4 | −1.0089 | 0.99933 | 0.99935 | 1 | 17518 | 0 | −1.0089 |
| LYPLA2 | 4 | 0.00067911 | 0.0024721 | 0.062157 | 718 | 4 | −0.90063 | 0.99932 | 0.99934 | 1 | 17517 | 0 | −0.90063 |
| TAF7 | 4 | 0.00068397 | 0.0024913 | 0.062553 | 719 | 4 | −1.3399 | 0.98863 | 0.98875 | 1 | 16701 | 0 | −1.3399 |
| PCBP1 | 4 | 0.00068794 | 0.0025088 | 0.062906 | 720 | 4 | −1.2888 | 0.99931 | 0.99933 | 1 | 17516 | 0 | −1.2888 |
| MRPS23 | 4 | 0.00069774 | 0.0025439 | 0.063691 | 721 | 4 | −2.0748 | 0.99714 | 0.99717 | 1 | 17215 | 0 | −2.0748 |
| NDUFAF3 | 4 | 0.00069855 | 0.0025472 | 0.063691 | 722 | 4 | −2.3599 | 0.9993 | 0.99932 | 1 | 17514 | 0 | −2.3599 |
| GSG2 | 4 | 0.00070123 | 0.0025527 | 0.06374 | 723 | 4 | −1.1046 | 0.9993 | 0.99931 | 1 | 17512 | 0 | −1.1046 |
| SLC35C2 | 4 | 0.00070415 | 0.0025637 | 0.063926 | 724 | 4 | −1.0582 | 0.9993 | 0.99931 | 1 | 17511 | 0 | −1.0582 |
| TSC1 | 4 | 0.00070831 | 0.0025785 | 0.064206 | 725 | 4 | −0.65859 | 0.99929 | 0.9993 | 1 | 17510 | 0 | −0.65859 |
| PPP6C | 4 | 0.00071322 | 0.0025944 | 0.064465 | 726 | 4 | −1.1257 | 0.99929 | 0.99929 | 1 | 17509 | 0 | −1.1257 |
| ZFP64 | 4 | 0.00071421 | 0.002596 | 0.064465 | 727 | 4 | −0.62215 | 0.99929 | 0.99929 | 1 | 17508 | 0 | −0.62215 |
| XRCC1 | 4 | 0.00072373 | 0.0026284 | 0.065117 | 728 | 3 | −0.68824 | 0.92258 | 0.92241 | 1 | 14878 | 0 | −0.68824 |
| RNASEH2C | 4 | 0.00072425 | 0.0026295 | 0.065117 | 729 | 4 | −0.78208 | 0.99882 | 0.99881 | 1 | 17424 | 0 | −0.78208 |
| TBCC | 4 | 0.00072583 | 0.0026355 | 0.065177 | 730 | 4 | −1.4886 | 0.99871 | 0.99871 | 1 | 17407 | 0 | −1.4886 |
| MAB21L3 | 4 | 0.00072988 | 0.0026498 | 0.06544 | 731 | 4 | −0.53416 | 0.99927 | 0.99928 | 1 | 17506 | 0 | −0.53416 |
| TTI2 | 4 | 0.00073215 | 0.0026602 | 0.065608 | 732 | 4 | −1.054 | 0.99892 | 0.99891 | 1 | 17443 | 0 | −1.054 |
| ANXA9 | 4 | 0.00073995 | 0.0026882 | 0.066207 | 733 | 4 | −1.1914 | 0.99926 | 0.99926 | 1 | 17505 | 0 | −1.1914 |
| TRMT5 | 4 | 0.00074139 | 0.0026926 | 0.066225 | 734 | 3 | −1.5469 | 0.91666 | 0.91646 | 1 | 14752 | 0 | −1.5469 |
| ELOF1 | 4 | 0.0007455 | 0.0027041 | 0.066417 | 735 | 4 | −0.85114 | 0.97822 | 0.97831 | 1 | 16303 | 0 | −0.85114 |
| PRRC2A | 4 | 0.00074758 | 0.0027085 | 0.066435 | 736 | 4 | −1.0352 | 0.99925 | 0.99926 | 1 | 17504 | 0 | −1.0352 |
| TNFRSF1A | 4 | 0.00075091 | 0.0027189 | 0.06651 | 737 | 4 | −0.59939 | 0.99925 | 0.99925 | 1 | 17503 | 0 | −0.59939 |
| NUDT1 | 4 | 0.0007527 | 0.002726 | 0.066594 | 738 | 4 | −0.78831 | 0.99925 | 0.99925 | 1 | 17502 | 0 | −0.78831 |
| NSMCE4A | 4 | 0.00075465 | 0.0027315 | 0.066638 | 739 | 3 | −0.8444 | 0.80707 | 0.83151 | 1 | 13201 | 1 | −0.8444 |
| GATC | 4 | 0.00076005 | 0.0027496 | 0.066989 | 740 | 3 | −1.0453 | 0.89638 | 0.89638 | 1 | 14382 | 1 | −1.0453 |
| PAGR1 | 4 | 0.00076443 | 0.0027633 | 0.067171 | 741 | 3 | −1.1352 | 0.90848 | 0.9082 | 1 | 14600 | 0 | −1.1352 |
| YRDC | 4 | 0.00076509 | 0.0027661 | 0.067171 | 742 | 4 | −2.0005 | 0.99923 | 0.99924 | 1 | 17501 | 0 | −2.0005 |
| SOD1 | 4 | 0.00076569 | 0.0027683 | 0.067171 | 743 | 4 | −2.93 | 0.98815 | 0.98825 | 1 | 16687 | 0 | −2.93 |
| NAA15 | 4 | 0.00076825 | 0.0027803 | 0.067373 | 744 | 3 | −2.0011 | 0.74432 | 0.7985 | 1 | 12662 | 1 | −2.0011 |
| PELP1 | 4 | 0.00077894 | 0.0028198 | 0.068228 | 745 | 3 | −1.8039 | 0.4568 | 0.61677 | 1 | 10070 | 1 | −1.8039 |
| RPIA | 4 | 0.00078106 | 0.0028264 | 0.068228 | 746 | 4 | −1.1905 | 0.99922 | 0.99921 | 1 | 17500 | 0 | −1.1905 |
| TRAF7 | 4 | 0.00078185 | 0.0028269 | 0.068228 | 747 | 4 | −1.1356 | 0.99922 | 0.99921 | 1 | 17499 | 0 | −1.1356 |
| SLMO2 | 4 | 0.00078317 | 0.0028324 | 0.068269 | 748 | 4 | −1.6766 | 0.99922 | 0.99921 | 1 | 17498 | 0 | −1.6766 |
| ZC3HC1 | 4 | 0.00078477 | 0.0028412 | 0.068389 | 749 | 4 | −0.90351 | 0.99886 | 0.99885 | 1 | 17430 | 0 | −0.90351 |
| MRRF | 4 | 0.0007914 | 0.0028664 | 0.068905 | 750 | 4 | −1.6046 | 0.99921 | 0.9992 | 1 | 17497 | 0 | −1.6046 |
| CLK2 | 4 | 0.00079246 | 0.0028719 | 0.068906 | 751 | 4 | −0.99004 | 0.99921 | 0.9992 | 1 | 17496 | 0 | −0.99004 |
| GTF3C5 | 4 | 0.00079368 | 0.0028741 | 0.068906 | 752 | 3 | −1.7471 | 0.94385 | 0.94371 | 1 | 15339 | 0 | −1.7471 |
| FDX1L | 4 | 0.00079759 | 0.0028889 | 0.069104 | 753 | 4 | −1.0328 | 0.99756 | 0.99759 | 1 | 17266 | 0 | −1.0328 |
| PCID2 | 4 | 0.00079806 | 0.0028927 | 0.069104 | 754 | 4 | −2.9537 | 0.99854 | 0.99856 | 1 | 17378 | 0 | −2.9537 |
| DIMT1 | 4 | 0.00079834 | 0.0028938 | 0.069104 | 755 | 4 | −0.66496 | 0.9992 | 0.99919 | 1 | 17494 | 0 | −0.66496 |
| TMX2 | 4 | 0.00081021 | 0.0029383 | 0.070043 | 756 | 4 | −1.1278 | 0.99919 | 0.99918 | 1 | 17493 | 0 | −1.1278 |
| COX4I1 | 4 | 0.00081102 | 0.002941 | 0.070043 | 757 | 4 | −1.704 | 0.99919 | 0.99918 | 1 | 17492 | 0 | −1.704 |
| PAICS | 4 | 0.00081282 | 0.0029476 | 0.070043 | 758 | 4 | −1.1297 | 0.97992 | 0.98001 | 1 | 16355 | 0 | −1.1297 |
| CAPZB | 4 | 0.00081339 | 0.0029487 | 0.070043 | 759 | 3 | −1.1145 | 0.46125 | 0.62052 | 1 | 10103 | 1 | −1.1145 |
| UGP2 | 4 | 0.00081646 | 0.0029619 | 0.070263 | 760 | 4 | −0.67406 | 0.99918 | 0.99917 | 1 | 17491 | 0 | −0.67406 |
| MTO1 | 4 | 0.00081908 | 0.0029739 | 0.070457 | 761 | 4 | −1.226 | 0.98435 | 0.98441 | 1 | 16523 | 0 | −1.226 |
| M1AP | 4 | 0.0008248 | 0.0029926 | 0.070806 | 762 | 3 | −1.0869 | 0.27745 | 0.46635 | 1 | 8126 | 1 | −1.0869 |
| HSPA8 | 4 | 0.00082963 | 0.0030068 | 0.07105 | 763 | 4 | −1.2064 | 0.99917 | 0.99916 | 1 | 17490 | 0 | −1.2064 |
| SMC6 | 4 | 0.00083304 | 0.0030183 | 0.071229 | 764 | 4 | −1.0215 | 0.99857 | 0.99858 | 1 | 17387 | 0 | −1.0215 |
| IDI1 | 4 | 0.00083697 | 0.0030321 | 0.071459 | 765 | 3 | −0.79733 | 0.47172 | 0.62938 | 1 | 10205 | 1 | −0.79733 |
| AATF | 4 | 0.00084324 | 0.0030537 | 0.071824 | 766 | 4 | −0.77201 | 0.99916 | 0.99914 | 1 | 17488 | 0 | −0.77201 |
| NUP210L | 4 | 0.00084435 | 0.0030589 | 0.071824 | 767 | 4 | −1.0109 | 0.99916 | 0.99914 | 1 | 17487 | 0 | −1.0109 |
| ACTR5 | 4 | 0.00084463 | 0.0030595 | 0.071824 | 768 | 4 | −1.2672 | 0.99916 | 0.99914 | 1 | 17486 | 0 | −1.2672 |
| MON2 | 4 | 0.00084659 | 0.0030666 | 0.071898 | 769 | 4 | −1.1963 | 0.99915 | 0.99914 | 1 | 17485 | 0 | −1.1963 |
| UBE3D | 4 | 0.00085081 | 0.0030847 | 0.072228 | 770 | 4 | −0.92806 | 0.99624 | 0.99632 | 1 | 17144 | 0 | −0.92806 |
| RPS5 | 4 | 0.00085362 | 0.003094 | 0.072353 | 771 | 4 | −1.8524 | 0.99915 | 0.99913 | 1 | 17484 | 0 | −1.8524 |
| EHMT2 | 4 | 0.00086024 | 0.0031204 | 0.072874 | 772 | 4 | −0.74526 | 0.99864 | 0.99866 | 1 | 17400 | 0 | −0.74526 |
| CPNE1 | 4 | 0.00086268 | 0.0031319 | 0.073049 | 773 | 4 | −0.88858 | 0.99914 | 0.99912 | 1 | 17483 | 0 | −0.88858 |
| DEXI | 4 | 0.00086467 | 0.0031384 | 0.073052 | 774 | 1 | −0.90554 | 0.99914 | 0.99912 | 1 | 17482 | 0 | −0.90554 |
| ICT1 | 4 | 0.00086496 | 0.0031401 | 0.073052 | 775 | 3 | −1.488 | 0.53174 | 0.68033 | 1 | 10932 | 1 | −1.488 |
| TUFM | 4 | 0.00087479 | 0.0031774 | 0.073824 | 776 | 4 | −0.99137 | 0.99844 | 0.99846 | 1 | 17364 | 0 | −0.99137 |
| MCTS1 | 4 | 0.00087669 | 0.003184 | 0.073882 | 777 | 4 | −1.038 | 0.99912 | 0.99911 | 1 | 17481 | 0 | −1.038 |
| ORC2 | 4 | 0.00088461 | 0.003207 | 0.074321 | 778 | 4 | −1.3343 | 0.99857 | 0.99858 | 1 | 17385 | 0 | −1.3343 |
| GRWD1 | 4 | 0.0008869 | 0.0032196 | 0.074518 | 779 | 3 | −0.99012 | 0.37432 | 0.54712 | 1 | 9163 | 1 | −0.99012 |
| NCAPG | 4 | 0.00089145 | 0.003235 | 0.074777 | 780 | 4 | −1.3798 | 0.99911 | 0.99909 | 1 | 17480 | 0 | −1.3798 |
| DHX9 | 4 | 0.00089291 | 0.0032399 | 0.074796 | 781 | 4 | −1.4599 | 0.99911 | 0.99909 | 1 | 17479 | 0 | −1.4599 |
| CTPS1 | 4 | 0.00089787 | 0.0032608 | 0.07509 | 782 | 4 | −0.69705 | 0.9991 | 0.99909 | 1 | 17478 | 0 | −0.69705 |
| SREBF1 | 4 | 0.00089817 | 0.0032618 | 0.07509 | 783 | 4 | −0.73624 | 0.9991 | 0.99909 | 1 | 17477 | 0 | −0.73624 |
| DNLZ | 4 | 0.00089969 | 0.0032651 | 0.07509 | 784 | 3 | −1.8763 | 0.95764 | 0.95755 | 1 | 15683 | 0 | −1.8763 |
| RPS16 | 4 | 0.00091231 | 0.0033085 | 0.075956 | 785 | 4 | −1.25 | 0.99909 | 0.99907 | 1 | 17476 | 0 | −1.25 |
| TAF3 | 4 | 0.0009131 | 0.0033112 | 0.075956 | 786 | 3 | −0.94732 | 0.94186 | 0.94172 | 1 | 15296 | 0 | −0.94732 |
| UBE2M | 4 | 0.00091678 | 0.003326 | 0.076199 | 787 | 3 | −2.1393 | 0.87526 | 0.87794 | 1 | 14044 | 1 | −2.1393 |
| DDX10 | 4 | 0.00093657 | 0.0034022 | 0.077739 | 788 | 4 | −1.667 | 0.99595 | 0.99603 | 1 | 17126 | 0 | −1.667 |
| EFNA3 | 4 | 0.00093897 | 0.0034077 | 0.077739 | 789 | 4 | −0.92058 | 0.99906 | 0.99904 | 1 | 17474 | 0 | −0.92058 |
| PFDN6 | 4 | 0.00093969 | 0.0034088 | 0.077739 | 790 | 4 | −1.4537 | 0.99766 | 0.99768 | 1 | 17279 | 0 | −1.4537 |
| TAF8 | 4 | 0.00094018 | 0.0034105 | 0.077739 | 791 | 4 | −1.0998 | 0.99906 | 0.99904 | 1 | 17473 | 0 | −1.0998 |
| RFWD3 | 4 | 0.000942 | 0.0034165 | 0.077778 | 792 | 4 | −0.84033 | 0.99906 | 0.99904 | 1 | 17472 | 0 | −0.84033 |
| SHC1 | 4 | 0.00094282 | 0.0034209 | 0.07778 | 793 | 4 | −1.0245 | 0.96456 | 0.96448 | 1 | 15859 | 0 | −1.0245 |
| TAF1B | 4 | 0.00094413 | 0.0034264 | 0.077807 | 794 | 4 | −0.59726 | 0.99906 | 0.99904 | 1 | 17471 | 0 | −0.59726 |
| OXER1 | 4 | 0.00094962 | 0.0034472 | 0.078074 | 795 | 4 | −1.1484 | 0.99905 | 0.99903 | 1 | 17469 | 0 | −1.1484 |
| PPP1R2 | 4 | 0.00094971 | 0.0034478 | 0.078074 | 796 | 4 | −1.1772 | 0.99902 | 0.999 | 1 | 17463 | 0 | −1.1772 |
| USP5 | 4 | 0.0009516 | 0.0034554 | 0.078074 | 797 | 4 | −0.98092 | 0.97083 | 0.97076 | 1 | 16057 | 0 | −0.98092 |
| AAGAB | 4 | 0.00095176 | 0.0034554 | 0.078074 | 798 | 4 | −0.46264 | 0.99905 | 0.99903 | 1 | 17468 | 0 | −0.46264 |
| LSM7 | 4 | 0.00095359 | 0.0034604 | 0.078088 | 799 | 4 | −1.2742 | 0.99905 | 0.99903 | 1 | 17467 | 0 | −1.2742 |
| BRK1 | 4 | 0.00095758 | 0.0034724 | 0.078262 | 800 | 4 | −0.89089 | 0.99904 | 0.99903 | 1 | 17466 | 0 | −0.89089 |
| PHIP | 4 | 0.00096251 | 0.0034829 | 0.078399 | 801 | 4 | −1.0139 | 0.99904 | 0.99902 | 1 | 17465 | 0 | −1.0139 |
| SELENBP1 | 4 | 0.00097087 | 0.0035103 | 0.078918 | 802 | 4 | −0.55949 | 0.99903 | 0.99901 | 1 | 17464 | 0 | −0.55949 |
| SLC25A3 | 4 | 0.00097823 | 0.0035284 | 0.079226 | 803 | 3 | −1.3134 | 0.59836 | 0.73821 | 1 | 11754 | 1 | −1.3134 |
| EIF3H | 4 | 0.00098115 | 0.0035361 | 0.079226 | 804 | 4 | −0.79491 | 0.99902 | 0.999 | 1 | 17462 | 0 | −0.79491 |
| TSHB | 4 | 0.00098147 | 0.0035372 | 0.079226 | 805 | 4 | −0.50161 | 0.99902 | 0.999 | 1 | 17461 | 0 | −0.50161 |
| HMGCS1 | 4 | 0.00098554 | 0.0035547 | 0.079521 | 806 | 4 | −1.2776 | 0.99901 | 0.999 | 1 | 17460 | 0 | −1.2776 |
| ERCC1 | 4 | 0.00099114 | 0.0035734 | 0.079703 | 807 | 4 | −1.0997 | 0.99684 | 0.99688 | 1 | 17188 | 0 | −1.0997 |
| ASNA1 | 4 | 0.00099179 | 0.0035756 | 0.079703 | 808 | 4 | −1.1529 | 0.98908 | 0.98921 | 1 | 16725 | 0 | −1.1529 |
| MRPS35 | 4 | 0.00099184 | 0.0035761 | 0.079703 | 809 | 4 | −1.1023 | 0.99901 | 0.99899 | 1 | 17459 | 0 | −1.1023 |
| MRPS10 | 4 | 0.00099405 | 0.0035854 | 0.079812 | 810 | 4 | −1.2194 | 0.99901 | 0.99899 | 1 | 17458 | 0 | −1.2194 |
| HYPK | 4 | 0.001008 | 0.0036348 | 0.080811 | 811 | 4 | −1.9344 | 0.99899 | 0.99898 | 1 | 17457 | 0 | −1.9344 |
| RAC1 | 4 | 0.0010099 | 0.0036408 | 0.080846 | 812 | 4 | −1.0547 | 0.99899 | 0.99898 | 1 | 17455 | 0 | −1.0547 |
| SMARCB1 | 4 | 0.0010126 | 0.0036534 | 0.081026 | 813 | 4 | −0.9364 | 0.995 | 0.99509 | 1 | 17056 | 0 | −0.9364 |
| RPAIN | 4 | 0.0010212 | 0.0036825 | 0.081483 | 814 | 3 | −0.95769 | 0.68847 | 0.77393 | 1 | 12281 | 1 | −0.95769 |
| C9orf78 | 4 | 0.0010215 | 0.003683 | 0.081483 | 815 | 4 | −1.0145 | 0.99898 | 0.99897 | 1 | 17453 | 0 | −1.0145 |
| DYNLRB1 | 4 | 0.001026 | 0.0036995 | 0.081719 | 816 | 4 | −1.6473 | 0.99897 | 0.99897 | 1 | 17452 | 0 | −1.6473 |
| METTL17 | 4 | 0.0010277 | 0.0037028 | 0.081719 | 817 | 3 | −1.4151 | 0.76189 | 0.80713 | 1 | 12787 | 1 | −1.4151 |
| VRK1 | 4 | 0.0010298 | 0.0037121 | 0.081825 | 818 | 4 | −0.92793 | 0.99437 | 0.99449 | 1 | 17019 | 0 | −0.92793 |
| RPL14 | 4 | 0.0010309 | 0.0037182 | 0.081858 | 819 | 4 | −1.3904 | 0.99897 | 0.99896 | 1 | 17451 | 0 | −1.3904 |
| PDCL | 4 | 0.0010341 | 0.0037357 | 0.082069 | 820 | 1 | −1.1249 | 0.99897 | 0.99896 | 1 | 17450 | 0 | −1.1249 |
| PEX5 | 4 | 0.0010345 | 0.0037368 | 0.082069 | 821 | 4 | −0.5666 | 0.99897 | 0.99896 | 1 | 17449 | 0 | −0.5666 |
| UBE2J2 | 4 | 0.0010371 | 0.003745 | 0.082149 | 822 | 4 | −0.91913 | 0.99637 | 0.99644 | 1 | 17156 | 0 | −0.91913 |
| GEMIN2 | 4 | 0.0010452 | 0.003773 | 0.082662 | 823 | 2 | −0.65386 | 0.55629 | 0.70173 | 1 | 11225 | 2 | −0.65386 |
| SLAMF1 | 4 | 0.0010518 | 0.0037999 | 0.08315 | 824 | 4 | −0.53514 | 0.99895 | 0.99894 | 1 | 17446 | 0 | −0.53514 |
| ACO2 | 4 | 0.0010551 | 0.0038092 | 0.083253 | 825 | 3 | −3.033 | 0.0087501 | 0.029555 | 0.521995 | 998 | 1 | −3.033 |
| RRP9 | 4 | 0.0010608 | 0.0038311 | 0.083632 | 826 | 4 | −1.2121 | 0.99894 | 0.99893 | 1 | 17445 | 0 | −1.2121 |
| MRPL19 | 4 | 0.0010639 | 0.0038415 | 0.083758 | 827 | 4 | −1.135 | 0.99596 | 0.99604 | 1 | 17128 | 0 | −1.135 |
| TRA2B | 4 | 0.0010693 | 0.0038602 | 0.083974 | 828 | 4 | −1.4304 | 0.98246 | 0.98253 | 1 | 16444 | 0 | −1.4304 |
| ELP6 | 4 | 0.0010695 | 0.0038607 | 0.083974 | 829 | 4 | −1.4156 | 0.99717 | 0.9972 | 1 | 17217 | 0 | −1.4156 |
| ARGLU1 | 4 | 0.0010727 | 0.0038756 | 0.084194 | 830 | 4 | −1.0371 | 0.97247 | 0.97243 | 1 | 16109 | 0 | −1.0371 |
| CTSK | 4 | 0.0010748 | 0.0038832 | 0.08426 | 831 | 4 | −0.85572 | 0.99893 | 0.99891 | 1 | 17444 | 0 | −0.85572 |
| COA3 | 4 | 0.0010796 | 0.0039046 | 0.084622 | 832 | 3 | −2.0689 | 0.76456 | 0.80847 | 1 | 12815 | 1 | −2.0689 |
| SLAMF9 | 4 | 0.0010899 | 0.0039381 | 0.085245 | 833 | 4 | −0.9445 | 0.99891 | 0.9989 | 1 | 17441 | 0 | −0.9445 |
| MIS12 | 4 | 0.0010918 | 0.0039458 | 0.085309 | 834 | 2 | −0.73127 | 0.40699 | 0.57462 | 1 | 9518 | 2 | −0.73127 |
| RMI1 | 4 | 0.0011057 | 0.003999 | 0.086252 | 835 | 4 | −1.1467 | 0.987 | 0.98708 | 1 | 16635 | 0 | −1.1467 |
| BRF1 | 4 | 0.0011059 | 0.003999 | 0.086252 | 836 | 4 | −0.93033 | 0.99889 | 0.99888 | 1 | 17439 | 0 | −0.93033 |
| SPATA25 | 4 | 0.0011156 | 0.0040308 | 0.086835 | 837 | 4 | −0.63266 | 0.99888 | 0.99887 | 1 | 17438 | 0 | −0.63266 |
| SNRPB2 | 4 | 0.0011211 | 0.0040467 | 0.087073 | 838 | 1 | −0.60813 | 0.99888 | 0.99887 | 1 | 17437 | 0 | −0.60813 |
| HCN3 | 4 | 0.0011249 | 0.0040631 | 0.087088 | 839 | 4 | −0.59274 | 0.99888 | 0.99886 | 1 | 17436 | 0 | −0.59274 |
| SRPRB | 4 | 0.001128 | 0.0040735 | 0.087088 | 840 | 4 | −0.96835 | 0.99887 | 0.99886 | 1 | 17435 | 0 | −0.96835 |
| LBP | 4 | 0.0011288 | 0.0040746 | 0.087088 | 841 | 3 | −0.84134 | 0.3876 | 0.55828 | 1 | 9312 | 1 | −0.84134 |
| CHD8 | 4 | 0.0011294 | 0.0040757 | 0.087088 | 842 | 4 | −1.0379 | 0.99887 | 0.99886 | 1 | 17434 | 0 | −1.0379 |
| PPP1R11 | 4 | 0.0011295 | 0.0040757 | 0.087088 | 843 | 4 | −1.14 | 0.99535 | 0.99545 | 1 | 17079 | 0 | −1.14 |
| ZSCAN9 | 4 | 0.0011298 | 0.0040763 | 0.087088 | 844 | 4 | −0.51336 | 0.99887 | 0.99886 | 1 | 17433 | 0 | −0.51336 |
| EIF3I | 4 | 0.0011364 | 0.0040993 | 0.087476 | 845 | 4 | −0.8986 | 0.99886 | 0.99885 | 1 | 17432 | 0 | −0.8986 |
| HUS1 | 4 | 0.0011381 | 0.0041053 | 0.087502 | 846 | 4 | −1.1866 | 0.99886 | 0.99885 | 1 | 17431 | 0 | −1.1866 |
| XYLT2 | 4 | 0.0011409 | 0.0041174 | 0.087655 | 847 | 4 | −0.60205 | 0.99886 | 0.99885 | 1 | 17429 | 0 | −0.60205 |
| CCNC | 4 | 0.0011429 | 0.0041229 | 0.087669 | 848 | 4 | −0.93316 | 0.99758 | 0.9976 | 1 | 17272 | 0 | −0.93316 |
| UBAP2L | 4 | 0.0011472 | 0.0041361 | 0.087845 | 849 | 4 | −1.2822 | 0.98928 | 0.9894 | 1 | 16737 | 0 | −1.2822 |
| CTNNBL1 | 4 | 0.0011509 | 0.0041536 | 0.088069 | 850 | 4 | −1.2305 | 0.99028 | 0.99036 | 1 | 16789 | 0 | −1.2305 |
| VHLL | 4 | 0.0011511 | 0.0041564 | 0.088069 | 851 | 4 | −1.0639 | 0.99885 | 0.99884 | 1 | 17428 | 0 | −1.0639 |
| CENPN | 4 | 0.0011525 | 0.0041613 | 0.08807 | 852 | 4 | −1.4516 | 0.99885 | 0.99884 | 1 | 17427 | 0 | −1.4516 |
| VMA21 | 4 | 0.0011569 | 0.0041777 | 0.088315 | 853 | 4 | −0.66024 | 0.99793 | 0.99797 | 1 | 17310 | 0 | −0.66024 |
| MGAT1 | 4 | 0.0011593 | 0.0041926 | 0.088352 | 854 | 3 | −1.213 | 0.79734 | 0.8259 | 1 | 13108 | 1 | −1.213 |
| RHNO1 | 4 | 0.0011596 | 0.0041936 | 0.088352 | 855 | 4 | −0.87147 | 0.99884 | 0.99883 | 1 | 17426 | 0 | −0.87147 |
| HYOU1 | 4 | 0.0011601 | 0.0041942 | 0.088352 | 856 | 4 | −1.7775 | 0.98398 | 0.98404 | 1 | 16503 | 0 | −1.7775 |
| UTP20 | 4 | 0.0011667 | 0.0042145 | 0.088676 | 857 | 4 | −1.3273 | 0.99883 | 0.99882 | 1 | 17425 | 0 | −1.3273 |
| EP400NL | 4 | 0.001174 | 0.0042419 | 0.089149 | 858 | 3 | −1.3322 | 0.95402 | 0.95387 | 1 | 15585 | 0 | −1.3322 |
| NSF | 4 | 0.0011832 | 0.0042699 | 0.089633 | 859 | 4 | −1.3194 | 0.99882 | 0.9988 | 1 | 17423 | 0 | −1.3194 |
| TAF6 | 4 | 0.0011889 | 0.0042891 | 0.089931 | 860 | 3 | −0.9377 | 0.95362 | 0.95348 | 1 | 15578 | 0 | −0.9377 |
| NAF1 | 4 | 0.0011904 | 0.0042946 | 0.089942 | 861 | 4 | −1.1872 | 0.99245 | 0.99256 | 1 | 16906 | 0 | −1.1872 |
| RPS11 | 4 | 0.0011977 | 0.0043187 | 0.090342 | 862 | 4 | −3.2715 | 0.9988 | 0.99879 | 1 | 17422 | 0 | −3.2715 |
| SNRNP40 | 4 | 0.0012021 | 0.0043384 | 0.09065 | 863 | 3 | −1.0047 | 0.91381 | 0.91359 | 1 | 14697 | 0 | −1.0047 |
| RFC2 | 4 | 0.0012058 | 0.0043543 | 0.09085 | 864 | 3 | −1.3973 | 0.78637 | 0.81986 | 1 | 13012 | 1 | −1.3973 |
| ZNF213 | 4 | 0.0012075 | 0.0043615 | 0.09085 | 865 | 4 | −0.55307 | 0.99879 | 0.99878 | 1 | 17421 | 0 | −0.55307 |
| ATP5D | 4 | 0.001208 | 0.0043631 | 0.09085 | 866 | 3 | −1.307 | 0.73422 | 0.79376 | 1 | 12582 | 1 | −1.307 |
| UBE2I | 4 | 0.0012102 | 0.0043713 | 0.090917 | 867 | 4 | −1.6639 | 0.99366 | 0.99376 | 1 | 16971 | 0 | −1.6639 |
| PET100 | 4 | 0.0012162 | 0.0043933 | 0.091268 | 868 | 3 | −1.122 | 0.049172 | 0.13516 | 0.729284 | 3302 | 1 | −1.122 |
| UQCRQ | 4 | 0.001219 | 0.004401 | 0.091322 | 869 | 3 | −1.3067 | 0.85433 | 0.86198 | 1 | 13765 | 1 | −1.3067 |
| DDX46 | 4 | 0.0012315 | 0.0044553 | 0.092343 | 870 | 4 | −1.6926 | 0.99877 | 0.99876 | 1 | 17419 | 0 | −1.6926 |
| TIMM13 | 4 | 0.0012352 | 0.0044712 | 0.092566 | 871 | 4 | −1.2322 | 0.99876 | 0.99876 | 1 | 17417 | 0 | −1.2322 |
| BUB1B | 4 | 0.0012371 | 0.0044783 | 0.092608 | 872 | 4 | −0.67873 | 0.99876 | 0.99876 | 1 | 17416 | 0 | −0.67873 |
| SART3 | 4 | 0.0012464 | 0.0045161 | 0.093199 | 873 | 4 | −1.6861 | 0.99875 | 0.99875 | 1 | 17415 | 0 | −1.6861 |
| RHOC | 4 | 0.0012466 | 0.0045172 | 0.093199 | 874 | 4 | −1.0179 | 0.99576 | 0.99584 | 1 | 17112 | 0 | −1.0179 |
| PYROXD1 | 4 | 0.0012557 | 0.0045375 | 0.093427 | 875 | 3 | −1.3113 | 0.28661 | 0.47391 | 1 | 8218 | 1 | −1.3113 |
| TIGD5 | 4 | 0.0012562 | 0.0045386 | 0.093427 | 876 | 4 | −0.80756 | 0.99874 | 0.99874 | 1 | 17414 | 0 | −0.80756 |
| SPCS2 | 4 | 0.0012577 | 0.0045457 | 0.093467 | 877 | 4 | −1.3385 | 0.99874 | 0.99873 | 1 | 17413 | 0 | −1.3385 |
| FAM161A | 4 | 0.0012713 | 0.0045978 | 0.094431 | 878 | 4 | −0.48057 | 0.99873 | 0.99872 | 1 | 17412 | 0 | −0.48057 |
| SNRPD1 | 4 | 0.0012778 | 0.004617 | 0.094718 | 879 | 4 | −1.0138 | 0.99872 | 0.99872 | 1 | 17411 | 0 | −1.0138 |
| CRKL | 4 | 0.0012827 | 0.0046329 | 0.094851 | 880 | 4 | −0.6146 | 0.99872 | 0.99872 | 1 | 17410 | 0 | −0.6146 |
| MED15 | 4 | 0.0012831 | 0.004634 | 0.094851 | 881 | 4 | −0.98567 | 0.99872 | 0.99872 | 1 | 17409 | 0 | −0.98567 |
| ACTR3 | 4 | 0.0012869 | 0.0046494 | 0.095057 | 882 | 4 | −0.97415 | 0.99871 | 0.99871 | 1 | 17408 | 0 | −0.97415 |
| MPLKIP | 4 | 0.0012943 | 0.0046724 | 0.09542 | 883 | 4 | −1.043 | 0.99871 | 0.99871 | 1 | 17406 | 0 | −1.043 |
| NDUFB10 | 4 | 0.0012976 | 0.0046845 | 0.095559 | 884 | 4 | −2.8548 | 0.99752 | 0.99755 | 1 | 17261 | 0 | −2.8548 |
| WDR4 | 4 | 0.0012991 | 0.0046916 | 0.095596 | 885 | 4 | −0.85318 | 0.98547 | 0.98554 | 1 | 16567 | 0 | −0.85318 |
| SLC16A1 | 4 | 0.001302 | 0.0047037 | 0.095734 | 886 | 4 | −0.5697 | 0.9987 | 0.9987 | 1 | 17405 | 0 | −0.5697 |
| MRPS18C | 4 | 0.0013151 | 0.0047503 | 0.096574 | 887 | 4 | −1.2911 | 0.99763 | 0.99765 | 1 | 17275 | 0 | −1.2911 |
| PABPC1 | 4 | 0.0013193 | 0.0047646 | 0.096755 | 888 | 3 | −1.0225 | 0.56631 | 0.71036 | 1 | 11342 | 1 | −1.0225 |
| C17orf89 | 4 | 0.0013263 | 0.004792 | 0.097202 | 889 | 4 | −1.0402 | 0.9726 | 0.97256 | 1 | 16113 | 0 | −1.0402 |
| DICER1 | 4 | 0.0013285 | 0.0047975 | 0.097204 | 890 | 4 | −0.5804 | 0.99867 | 0.99868 | 1 | 17404 | 0 | −0.5804 |
| CD48 | 4 | 0.0013342 | 0.0048123 | 0.097321 | 891 | 4 | −1.0378 | 0.99866 | 0.99868 | 1 | 17403 | 0 | −1.0378 |
| EEFSEC | 4 | 0.001335 | 0.004815 | 0.097321 | 892 | 3 | −1.1266 | 0.30578 | 0.48967 | 1 | 8434 | 1 | −1.1266 |
| RPUSD1 | 4 | 0.0013368 | 0.0048194 | 0.097321 | 893 | 4 | −0.49279 | 0.99866 | 0.99868 | 1 | 17402 | 0 | −0.49279 |
| CHCHD2 | 4 | 0.001349 | 0.0048649 | 0.09813 | 894 | 4 | −0.6843 | 0.99865 | 0.99866 | 1 | 17401 | 0 | −0.6843 |
| RCC1 | 4 | 0.0013547 | 0.0048858 | 0.098441 | 895 | 3 | −0.94625 | 0.34494 | 0.52243 | 1 | 8845 | 1 | −0.94625 |
| RFX5 | 4 | 0.0013602 | 0.0049094 | 0.098806 | 896 | 4 | −0.89161 | 0.99738 | 0.99741 | 1 | 17244 | 0 | −0.89161 |
| WAPAL | 4 | 0.0013638 | 0.0049198 | 0.098806 | 897 | 4 | −1.1476 | 0.99864 | 0.99865 | 1 | 17398 | 0 | −1.1476 |
| GINS2 | 4 | 0.0013647 | 0.004922 | 0.098806 | 898 | 4 | −1.8987 | 0.99812 | 0.99816 | 1 | 17330 | 0 | −1.8987 |
| VPS52 | 4 | 0.0013658 | 0.0049258 | 0.098806 | 899 | 4 | −0.67123 | 0.99863 | 0.99865 | 1 | 17397 | 0 | −0.67123 |
| EFNA4 | 4 | 0.0013702 | 0.0049368 | 0.098916 | 900 | 4 | −0.77873 | 0.99863 | 0.99864 | 1 | 17396 | 0 | −0.77873 |
| MRPS2 | 4 | 0.0013754 | 0.004951 | 0.099092 | 901 | 4 | −1.0131 | 0.99722 | 0.99725 | 1 | 17224 | 0 | −1.0131 |
| WDR43 | 4 | 0.0013868 | 0.0049905 | 0.099716 | 902 | 4 | −2.2481 | 0.99861 | 0.99863 | 1 | 17395 | 0 | −2.2481 |
| DDA1 | 4 | 0.0013888 | 0.0049971 | 0.099716 | 903 | 4 | −0.58372 | 0.99861 | 0.99862 | 1 | 17394 | 0 | −0.58372 |
| TINF2 | 4 | 0.0013891 | 0.0049988 | 0.099716 | 904 | 3 | −1.5164 | 0.91113 | 0.91092 | 1 | 14658 | 0 | −1.5164 |
| TGIF1 | 4 | 0.0013929 | 0.0050103 | 0.099835 | 905 | 4 | −0.77516 | 0.99861 | 0.99862 | 1 | 17393 | 0 | −0.77516 |
| CYCS | 1 | 0.0027593 | 0.0027134 | 0.066466 | 1138 | 1 | −2.1598 | 0.99724 | 0.99729 | 1 | 17228 | 0 | −2.1598 |

H2030 TNO SL Gene FDR 0.1

| | | | | | | | | | | | | | |
|---|---|---|---|---|---|---|---|---|---|---|---|---|---|
| SPTBN5 | 4 | 1.52E−06 | 4.66E−06 | 0.035891 | 1 | 3 | −0.68549 | 0.88246 | 0.88199 | 0.999998 | 15844 | 0 | −0.68549 |
| DNAJC9 | 4 | 2.06E−06 | 5.21E−06 | 0.035891 | 2 | 3 | −1.0104 | 0.5486 | 0.63414 | 0.999998 | 11301 | 1 | −1.0104 |
| BAP1 | 4 | 2.55E−06 | 6.86E−06 | 0.035891 | 3 | 4 | −0.83823 | 1 | 1 | 0.999998 | 18051 | 0 | −0.83823 |
| NUP54 | 4 | 2.67E−06 | 7.95E−06 | 0.035891 | 4 | 3 | −1.0698 | 0.95903 | 0.95892 | 0.999998 | 17266 | 0 | −1.0698 |
| PTBP1 | 4 | 6.28E−06 | 2.00E−05 | 0.061528 | 5 | 4 | −0.64197 | 0.98854 | 0.98865 | 0.999998 | 17808 | 0 | −0.64197 |
| ILF3 | 4 | 7.36E−06 | 2.33E−05 | 0.061528 | 6 | 3 | −1.0223 | 0.062492 | 0.14511 | 0.985078 | 2614 | 1 | −1.0223 |
| ELMO2 | 4 | 7.74E−06 | 2.39E−05 | 0.061528 | 7 | 3 | −1.2708 | 0.022537 | 0.065054 | 0.963089 | 1205 | 1 | −1.2708 |

H2122 TNO SL Gene FDR 0.1

| | | | | | | | | | | | | | |
|---|---|---|---|---|---|---|---|---|---|---|---|---|---|
| RTCB | 4 | 2.74E−09 | 2.74E−07 | 0.00045 | 1 | 4 | −1.6321 | 1 | 1 | 1 | 18053 | 0 | −1.6321 |
| ENO1 | 4 | 1.01E−08 | 2.74E−07 | 0.00045 | 2 | 4 | −2.7015 | 1 | 1 | 1 | 17996 | 0 | −2.7015 |
| GAPDH | 4 | 1.60E−08 | 2.74E−07 | 0.00045 | 3 | 4 | −1.6074 | 1 | 1 | 1 | 18052 | 0 | −1.6074 |
| MARS2 | 4 | 2.42E−08 | 2.74E−07 | 0.00045 | 4 | 4 | −2.4242 | 1 | 1 | 1 | 18051 | 0 | −2.4242 |
| ATP6V1F | 4 | 8.05E−08 | 2.74E−07 | 0.00045 | 5 | 4 | −1.5024 | 1 | 1 | 1 | 18050 | 0 | −1.5024 |
| PRMT5 | 4 | 9.79E−08 | 2.74E−07 | 0.00045 | 6 | 4 | −1.6074 | 1 | 1 | 1 | 18049 | 0 | −1.6074 |
| COQ2 | 4 | 1.06E−07 | 2.74E−07 | 0.00045 | 7 | 3 | −1.9783 | 0.97013 | 0.97005 | 1 | 16326 | 0 | −1.9783 |
| DBR1 | 4 | 1.35E−07 | 2.74E−07 | 0.00045 | 8 | 4 | −2.1272 | 1 | 1 | 1 | 18048 | 0 | −2.1272 |
| FKBPL | 4 | 1.55E−07 | 2.74E−07 | 0.00045 | 9 | 4 | −1.5952 | 1 | 1 | 1 | 18047 | 0 | −1.5952 |
| LYRM4 | 4 | 1.60E−07 | 2.74E−07 | 0.00045 | 10 | 4 | −1.9019 | 1 | 1 | 1 | 18046 | 0 | −1.9019 |
| HIRA | 4 | 1.76E−07 | 2.74E−07 | 0.00045 | 11 | 4 | −1.4421 | 1 | 1 | 1 | 18045 | 0 | −1.4421 |
| RCL1 | 4 | 2.10E−07 | 8.23E−07 | 0.000874 | 12 | 4 | −2.1066 | 1 | 1 | 1 | 18044 | 0 | −2.1066 |
| COA6 | 4 | 2.22E−07 | 8.23E−07 | 0.000874 | 13 | 4 | −3.3507 | 1 | 1 | 1 | 18043 | 0 | −3.3507 |
| WRB | 4 | 2.40E−07 | 8.23E−07 | 0.000874 | 14 | 4 | −2.5995 | 1 | 1 | 1 | 18042 | 0 | −2.5995 |
| TBCB | 4 | 2.47E−07 | 8.23E−07 | 0.000874 | 15 | 4 | −1.7201 | 1 | 1 | 1 | 18041 | 0 | −1.7201 |
| CPOX | 4 | 2.73E−07 | 8.23E−07 | 0.000874 | 16 | 4 | −2.2763 | 1 | 1 | 1 | 18040 | 0 | −2.2763 |
| SPATA5 | 4 | 2.74E−07 | 8.23E−07 | 0.000874 | 17 | 4 | −1.2811 | 1 | 1 | 1 | 18039 | 0 | −1.2811 |
| RBBP5 | 4 | 3.96E−07 | 1.37E−06 | 0.001238 | 18 | 4 | −1.6999 | 1 | 1 | 1 | 18038 | 0 | −1.6999 |
| DTYMK | 4 | 4.10E−07 | 1.37E−06 | 0.001238 | 19 | 4 | −2.6566 | 1 | 1 | 1 | 18037 | 0 | −2.6566 |
| GEMIN7 | 4 | 4.87E−07 | 1.37E−06 | 0.001238 | 20 | 4 | −1.6619 | 1 | 1 | 1 | 17995 | 0 | −1.6619 |
| PTPMT1 | 4 | 5.24E−07 | 1.92E−06 | 0.001238 | 21 | 4 | −1.602 | 1 | 1 | 1 | 18036 | 0 | −1.602 |
| ANAPC11 | 4 | 5.52E−07 | 1.92E−06 | 0.001238 | 22 | 4 | −1.835 | 1 | 1 | 1 | 18035 | 0 | −1.835 |
| POLR2L | 4 | 5.66E−07 | 1.92E−06 | 0.001238 | 23 | 4 | −2.4502 | 1 | 1 | 1 | 18034 | 0 | −2.4502 |
| DKC1 | 4 | 6.02E−07 | 1.92E−06 | 0.001238 | 24 | 4 | −2.2513 | 1 | 1 | 1 | 18033 | 0 | −2.2513 |
| RNMT | 4 | 6.69E−07 | 1.92E−06 | 0.001238 | 25 | 4 | −1.5797 | 1 | 1 | 1 | 18032 | 0 | −1.5797 |
| PPP1R8 | 4 | 7.00E−07 | 1.92E−06 | 0.001238 | 26 | 4 | −1.9097 | 1 | 1 | 1 | 18031 | 0 | −1.9097 |
| HSD17B10 | 4 | 7.08E−07 | 1.92E−06 | 0.001238 | 27 | 4 | −2.1168 | 1 | 1 | 1 | 18030 | 0 | −2.1168 |
| UBA1 | 4 | 7.14E−07 | 1.92E−06 | 0.001238 | 28 | 4 | −2.1726 | 1 | 1 | 1 | 18029 | 0 | −2.1726 |
| DOLK | 4 | 8.21E−07 | 2.47E−06 | 0.001536 | 29 | 4 | −2.0488 | 0.99993 | 0.99994 | 1 | 17843 | 0 | −2.0488 |
| HUWE1 | 4 | 9.10E−07 | 3.02E−06 | 0.00157 | 30 | 4 | −1.5649 | 1 | 1 | 1 | 18028 | 0 | −1.5649 |
| ALG1 | 4 | 9.15E−07 | 3.02E−06 | 0.00157 | 31 | 4 | −1.4217 | 1 | 1 | 1 | 18027 | 0 | −1.4217 |
| UROD | 4 | 9.50E−07 | 3.02E−06 | 0.00157 | 32 | 3 | −1.9556 | 0.71946 | 0.78011 | 1 | 12966 | 1 | −1.9556 |
| POLR3H | 4 | 9.61E−07 | 3.02E−06 | 0.00157 | 33 | 4 | −2.5055 | 1 | 1 | 1 | 18026 | 0 | −2.5055 |
| LIN52 | 4 | 9.84E−07 | 3.02E−06 | 0.00157 | 34 | 4 | −1.2007 | 1 | 1 | 1 | 18025 | 0 | −1.2007 |
| GLS | 4 | 1.04E−06 | 3.56E−06 | 0.00157 | 35 | 4 | −1.3942 | 1 | 1 | 1 | 18024 | 0 | −1.3942 |
| NAA25 | 4 | 1.05E−06 | 3.56E−06 | 0.00157 | 36 | 4 | −1.6251 | 1 | 1 | 1 | 18023 | 0 | −1.6251 |
| PGD | 4 | 1.07E−06 | 3.56E−06 | 0.00157 | 37 | 4 | −1.4678 | 1 | 1 | 1 | 18022 | 0 | −1.4678 |
| TSEN2 | 4 | 1.09E−06 | 3.56E−06 | 0.00157 | 38 | 4 | −1.7079 | 0.99991 | 0.99992 | 1 | 17827 | 0 | −1.7079 |
| NDUFS2 | 4 | 1.10E−06 | 3.56E−06 | 0.00157 | 39 | 4 | −1.494 | 1 | 1 | 1 | 18021 | 0 | −1.494 |
| RNASEH2A | 4 | 1.14E−06 | 3.56E−06 | 0.00157 | 40 | 4 | −1.1447 | 1 | 1 | 1 | 18020 | 0 | −1.1447 |
| GUK1 | 4 | 1.16E−06 | 3.56E−06 | 0.00157 | 41 | 4 | −1.8877 | 1 | 1 | 1 | 18019 | 0 | −1.8877 |
| CENPP | 4 | 1.30E−06 | 4.11E−06 | 0.001618 | 42 | 4 | −1.5456 | 1 | 1 | 1 | 18018 | 0 | −1.5456 |
| TSFM | 1 | 1.34E−06 | 4.66E−06 | 0.001618 | 43 | 4 | −1.7841 | 1 | 1 | 1 | 18017 | 0 | −1.7841 |
| MRPL13 | 4 | 1.35E−06 | 4.66E−06 | 0.001618 | 44 | 4 | −1.4286 | 1 | 1 | 1 | 18016 | 0 | −1.4286 |
| WDR77 | 4 | 1.35E−06 | 4.66E−06 | 0.001618 | 45 | 4 | −2.1471 | 1 | 1 | 1 | 18015 | 0 | −2.1471 |
| NELFB | 4 | 1.35E−06 | 4.66E−06 | 0.001618 | 46 | 4 | −2.2028 | 1 | 1 | 1 | 18014 | 0 | −2.2028 |
| DOHH | 4 | 1.38E−06 | 4.66E−06 | 0.001618 | 47 | 4 | −1.2116 | 1 | 1 | 1 | 18013 | 0 | −1.2116 |
| EXOSC5 | 4 | 1.54E−06 | 4.66E−06 | 0.001618 | 48 | 4 | −2.3824 | 1 | 1 | 1 | 18012 | 0 | −2.3824 |
| RPE | 4 | 1.61E−06 | 4.66E−06 | 0.001618 | 49 | 4 | −1.9189 | 1 | 1 | 1 | 18011 | 0 | −1.9189 |
| PGM3 | 4 | 1.62E−06 | 4.66E−06 | 0.001618 | 50 | 3 | −1.5229 | 0.96903 | 0.96893 | 1 | 16296 | 0 | −1.5229 |
| CSTF1 | 4 | 1.63E−06 | 4.66E−06 | 0.001618 | 51 | 4 | −1.4494 | 1 | 1 | 1 | 18010 | 0 | −1.4494 |
| MRPL53 | 4 | 1.68E−06 | 4.66E−06 | 0.001618 | 52 | 4 | −1.4494 | 1 | 1 | 1 | 18009 | 0 | −1.4494 |
| LSM10 | 4 | 1.73E−06 | 5.21E−06 | 0.001622 | 53 | 4 | −2.2874 | 1 | 1 | 1 | 18008 | 0 | −2.2874 |
| TIMMDC1 | 4 | 1.83E−06 | 5.21E−06 | 0.001622 | 54 | 4 | −1.3083 | 1 | 1 | 1 | 18007 | 0 | −1.3083 |
| RTEL1 | 4 | 1.89E−06 | 5.21E−06 | 0.001622 | 55 | 4 | −1.9991 | 1 | 1 | 1 | 18006 | 0 | −1.9991 |
| PTBP1 | 4 | 1.98E−06 | 5.21E−06 | 0.001622 | 56 | 4 | −1.2478 | 1 | 1 | 1 | 18005 | 0 | −1.2478 |
| WARS2 | 4 | 2.05E−06 | 5.21E−06 | 0.001622 | 57 | 4 | −2.1463 | 1 | 1 | 1 | 18004 | 0 | −2.1463 |
| IBA57 | 4 | 2.13E−06 | 5.21E−06 | 0.001622 | 58 | 4 | −1.5512 | 1 | 1 | 1 | 18003 | 0 | −1.5512 |
| EIF3F | 4 | 2.45E−06 | 5.76E−06 | 0.001762 | 59 | 4 | −1.6476 | 1 | 1 | 1 | 18002 | 0 | −1.6476 |
| LIPT1 | 4 | 2.52E−06 | 6.31E−06 | 0.001898 | 60 | 4 | −2.1116 | 1 | 1 | 1 | 18001 | 0 | −2.1116 |
| PET117 | 4 | 2.65E−06 | 7.40E−06 | 0.002191 | 61 | 4 | −1.6243 | 1 | 1 | 1 | 18000 | 0 | −1.6243 |
| UTP23 | 4 | 2.72E−06 | 8.50E−06 | 0.002475 | 62 | 4 | −2.7509 | 1 | 1 | 1 | 17999 | 0 | −2.7509 |
| TRAPPC1 | 4 | 2.97E−06 | 1.01E−05 | 0.002859 | 63 | 4 | −2.4111 | 1 | 1 | 1 | 17998 | 0 | −2.4111 |
| SLC35B1 | 4 | 2.99E−06 | 1.01E−05 | 0.002859 | 64 | 4 | −2.4556 | 1 | 1 | 1 | 17997 | 0 | −2.4556 |
| MRPS14 | 4 | 3.09E−06 | 1.07E−05 | 0.002859 | 65 | 3 | −2.0818 | 0.84305 | 0.85109 | 1 | 14123 | 1 | −2.0818 |
| ZNF236 | 4 | 3.19E−06 | 1.07E−05 | 0.002859 | 66 | 3 | −1.8392 | 0.90497 | 0.90464 | 1 | 15067 | 0 | −1.8392 |
| POLG2 | 4 | 3.25E−06 | 1.07E−05 | 0.002859 | 67 | 4 | −1.906 | 1 | 1 | 1 | 17994 | 0 | −1.906 |
| THG1L | 4 | 3.37E−06 | 1.12E−05 | 0.002859 | 68 | 4 | −1.7828 | 0.99533 | 0.99542 | 1 | 17181 | 0 | −1.7828 |
| RFT1 | 4 | 3.38E−06 | 1.12E−05 | 0.002859 | 69 | 4 | −1.4286 | 1 | 1 | 1 | 17993 | 0 | −1.4286 |
| HSCB | 4 | 3.40E−06 | 1.12E−05 | 0.002859 | 70 | 4 | −1.3665 | 1 | 1 | 1 | 17992 | 0 | −1.3665 |
| EXOSC4 | 4 | 3.55E−06 | 1.12E−05 | 0.002859 | 71 | 4 | −1.8945 | 1 | 1 | 1 | 17991 | 0 | −1.8945 |
| LIPT2 | 4 | 3.80E−06 | 1.18E−05 | 0.002957 | 72 | 4 | −1.7747 | 0.99977 | 0.99978 | 1 | 17738 | 0 | −1.7747 |
| RARS2 | 4 | 4.03E−06 | 1.23E−05 | 0.00301 | 73 | 4 | −1.8361 | 1 | 1 | 1 | 17990 | 0 | −1.8361 |
| MRPL12 | 4 | 4.07E−06 | 1.23E−05 | 0.00301 | 74 | 4 | −1.0366 | 1 | 1 | 1 | 17989 | 0 | −1.0366 |
| ASNA1 | 4 | 4.23E−06 | 1.29E−05 | 0.003061 | 75 | 4 | −1.9723 | 1 | 1 | 1 | 17988 | 0 | −1.9723 |
| CINP | 4 | 4.23E−06 | 1.29E−05 | 0.003061 | 76 | 2 | −1.1361 | 0.85483 | 0.85995 | 1 | 14275 | 1 | −1.1361 |
| RAD51D | 4 | 4.35E−06 | 1.34E−05 | 0.00315 | 77 | 4 | −1.3385 | 1 | 1 | 1 | 17987 | 0 | −1.3385 |
| PSMA3 | 4 | 4.42E−06 | 1.45E−05 | 0.003321 | 78 | 4 | −2.1701 | 1 | 1 | 1 | 17986 | 0 | −2.1701 |
| IMP3 | 4 | 4.48E−06 | 1.45E−05 | 0.003321 | 79 | 4 | −1.8952 | 1 | 1 | 1 | 17985 | 0 | −1.8952 |
| CDC123 | 4 | 4.51E−06 | 1.51E−05 | 0.003361 | 80 | 4 | −1.4131 | 1 | 1 | 1 | 17984 | 0 | −1.4131 |
| MRPS34 | 4 | 4.53E−06 | 1.51E−05 | 0.003361 | 81 | 4 | −1.6742 | 1 | 1 | 1 | 17983 | 0 | −1.6742 |
| NDUFA1 | 4 | 4.61E−06 | 1.56E−05 | 0.003441 | 82 | 4 | −1.6619 | 1 | 1 | 1 | 17982 | 0 | −1.6619 |
| PSMD2 | 4 | 4.80E−06 | 1.67E−05 | 0.003511 | 83 | 4 | −2.1519 | 1 | 1 | 1 | 17981 | 0 | −2.1519 |
| PPP1CB | 4 | 4.89E−06 | 1.67E−05 | 0.003511 | 84 | 3 | −1.5495 | 0.8333 | 0.84425 | 1 | 14013 | 1 | −1.5495 |
| SCO2 | 4 | 4.90E−06 | 1.67E−05 | 0.003511 | 85 | 4 | −1.4145 | 1 | 1 | 1 | 17980 | 0 | −1.4145 |
| RABGGTB | 4 | 4.98E−06 | 1.67E−05 | 0.003511 | 86 | 4 | −1.6353 | 1 | 1 | 1 | 17979 | 0 | −1.6353 |
| FECH | 4 | 5.94E−06 | 1.95E−05 | 0.003928 | 87 | 4 | −0.90103 | 0.99999 | 1 | 1 | 17978 | 0 | −0.90103 |
| TIMELESS | 4 | 5.98E−06 | 1.95E−05 | 0.003928 | 88 | 4 | −3.123 | 0.99999 | 1 | 1 | 17977 | 0 | −3.123 |
| ATP5A1 | 4 | 6.11E−06 | 1.95E−05 | 0.003928 | 89 | 4 | −1.5717 | 0.99999 | 1 | 1 | 17976 | 0 | −1.5717 |
| RFC5 | 4 | 6.22E−06 | 2.00E−05 | 0.003928 | 90 | 4 | −1.4188 | 0.99999 | 0.99999 | 1 | 17975 | 0 | −1.4188 |
| FAM96B | 4 | 6.22E−06 | 2.00E−05 | 0.003928 | 91 | 4 | −1.9359 | 0.99371 | 0.99382 | 1 | 17113 | 0 | −1.9359 |
| LARS2 | 4 | 6.41E−06 | 2.00E−05 | 0.003928 | 92 | 4 | −1.5564 | 0.97278 | 0.97275 | 1 | 16403 | 0 | −1.5564 |
| SDHB | 4 | 6.67E−06 | 2.06E−05 | 0.003992 | 93 | 4 | −2.0468 | 0.99999 | 0.99999 | 1 | 17974 | 0 | −2.0468 |
| ARL2 | 4 | 6.94E−06 | 2.17E−05 | 0.004161 | 94 | 4 | −1.6993 | 0.99999 | 0.99999 | 1 | 17973 | 0 | −1.6993 |
| GCSH | 4 | 7.25E−06 | 2.22E−05 | 0.004221 | 95 | 4 | −1.4916 | 0.99999 | 0.99999 | 1 | 17972 | 0 | −1.4916 |
| COX5B | 4 | 7.38E−06 | 2.33E−05 | 0.004338 | 96 | 4 | −1.453 | 0.99999 | 0.99999 | 1 | 17971 | 0 | −1.453 |
| CPSF4 | 4 | 7.46E−06 | 2.33E−05 | 0.004338 | 97 | 4 | −1.2626 | 0.99999 | 0.99999 | 1 | 17970 | 0 | −1.2626 |
| TONSL | 4 | 8.05E−06 | 2.61E−05 | 0.004799 | 98 | 4 | −1.9805 | 0.99999 | 0.99999 | 1 | 17969 | 0 | −1.9805 |
| NUDT21 | 4 | 8.43E−06 | 2.77E−05 | 0.004999 | 99 | 4 | −2.2219 | 0.99999 | 0.99999 | 1 | 17968 | 0 | −2.2219 |
| SAMM50 | 4 | 8.53E−06 | 2.82E−05 | 0.004999 | 100 | 4 | −2.3238 | 0.99999 | 0.99999 | 1 | 17967 | 0 | −2.3238 |
| PDPK1 | 4 | 8.60E−06 | 2.82E−05 | 0.004999 | 101 | 4 | −1.6833 | 0.99999 | 0.99999 | 1 | 17966 | 0 | −1.6833 |
| WAPAL | 4 | 8.66E−06 | 2.82E−05 | 0.004999 | 102 | 4 | −1.8423 | 0.99999 | 0.99999 | 1 | 17965 | 0 | −1.8423 |
| INTS9 | 4 | 8.80E−06 | 2.99E−05 | 0.005239 | 103 | 4 | −1.7738 | 0.99999 | 0.99999 | 1 | 17964 | 0 | −1.7738 |
| PHB | 4 | 9.17E−06 | 3.15E−05 | 0.005474 | 104 | 4 | −1.086 | 0.99975 | 0.99976 | 1 | 17725 | 0 | −1.086 |
| MECR | 4 | 9.24E−06 | 3.21E−05 | 0.005516 | 105 | 4 | −1.0517 | 0.99999 | 0.99999 | 1 | 17963 | 0 | −1.0517 |
| MCM5 | 4 | 9.59E−06 | 3.48E−05 | 0.005827 | 106 | 4 | −1.8346 | 0.99979 | 0.99979 | 1 | 17742 | 0 | −1.8346 |
| NOP9 | 4 | 1.01E−05 | 3.54E−05 | 0.005827 | 107 | 4 | −1.15 | 0.99999 | 0.99999 | 1 | 17962 | 0 | −1.15 |
| GTF2A2 | 4 | 1.02E−05 | 3.54E−05 | 0.005827 | 108 | 4 | −1.9142 | 0.99999 | 0.99999 | 1 | 17961 | 0 | −1.9142 |
| DDX10 | 4 | 1.03E−05 | 3.54E−05 | 0.005827 | 109 | 4 | −2.1247 | 0.99999 | 0.99999 | 1 | 17960 | 0 | −2.1247 |
| C3orf17 | 4 | 1.06E−05 | 3.59E−05 | 0.005827 | 110 | 4 | −2.1566 | 0.99999 | 0.99999 | 1 | 17959 | 0 | −2.1566 |
| FARSB | 4 | 1.07E−05 | 3.59E−05 | 0.005827 | 111 | 4 | −1.7712 | 0.99875 | 0.99875 | 1 | 17459 | 0 | −1.7712 |
| DNLZ | 4 | 1.10E−05 | 3.65E−05 | 0.005827 | 112 | 4 | −0.90794 | 0.99999 | 0.99999 | 1 | 17958 | 0 | −0.90794 |
| GPS1 | 4 | 1.11E−05 | 3.65E−05 | 0.005827 | 113 | 4 | −1.3442 | 0.99999 | 0.99999 | 1 | 17957 | 0 | −1.3442 |
| DAP3 | 4 | 1.12E−05 | 3.76E−05 | 0.005949 | 114 | 4 | −1.2842 | 0.99999 | 0.99999 | 1 | 17956 | 0 | −1.2842 |
| THAP1 | 4 | 1.12E−05 | 3.81E−05 | 0.005984 | 115 | 3 | −2.2068 | 0.76605 | 0.80357 | 1 | 13337 | 1 | −2.2068 |
| SNAPC3 | 4 | 1.13E−05 | 3.87E−05 | 0.006008 | 116 | 4 | −2.0712 | 0.99988 | 0.99988 | 1 | 17801 | 0 | −2.0712 |
| PDCD11 | 4 | 1.14E−05 | 3.89E−05 | 0.006008 | 117 | 4 | −2.1708 | 0.99999 | 0.99999 | 1 | 17955 | 0 | −2.1708 |
| PDCD2 | 4 | 1.21E−05 | 4.36E−05 | 0.006615 | 118 | 4 | −1.4795 | 0.99999 | 0.99999 | 1 | 17954 | 0 | −1.4795 |
| NAA20 | 4 | 1.21E−05 | 4.36E−05 | 0.006615 | 119 | 3 | −1.805 | 0.7343 | 0.78725 | 1 | 13083 | 1 | −1.805 |
| PPP2CA | 4 | 1.24E−05 | 4.58E−05 | 0.006802 | 120 | 4 | −1.8893 | 0.99999 | 0.99999 | 1 | 17953 | 0 | −1.8893 |
| GEMIN5 | 4 | 1.25E−05 | 4.63E−05 | 0.006802 | 121 | 4 | −1.6299 | 0.99999 | 0.99999 | 1 | 17952 | 0 | −1.6299 |
| TFRC | 4 | 1.25E−05 | 4.63E−05 | 0.006802 | 122 | 4 | −1.5573 | 0.99999 | 0.99999 | 1 | 17951 | 0 | −1.5573 |
| VARS2 | 4 | 1.26E−05 | 4.63E−05 | 0.006802 | 123 | 4 | −1.3975 | 0.99999 | 0.99999 | 1 | 17950 | 0 | −1.3975 |
| CPSF3L | 4 | 1.28E−05 | 4.74E−05 | 0.006907 | 124 | 4 | −2.0795 | 0.99999 | 0.99999 | 1 | 17949 | 0 | −2.0795 |
| AIFM1 | 4 | 1.35E−05 | 4.91E−05 | 0.007033 | 125 | 4 | −1.2458 | 0.99999 | 0.99999 | 1 | 17948 | 0 | −1.2458 |
| UBA3 | 4 | 1.36E−05 | 4.91E−05 | 0.007033 | 126 | 3 | −1.3333 | 0.3345 | 0.50768 | 1 | 8900 | 1 | −1.3333 |
| PTCD3 | 4 | 1.38E−05 | 5.02E−05 | 0.007045 | 127 | 4 | −1.448 | 0.99999 | 0.99999 | 1 | 17947 | 0 | −1.448 |
| PDSS2 | 4 | 1.41E−05 | 5.07E−05 | 0.007045 | 128 | 4 | −1.9834 | 0.99999 | 0.99999 | 1 | 17946 | 0 | −1.9834 |
| MRPL15 | 4 | 1.42E−05 | 5.07E−05 | 0.007045 | 129 | 4 | −1.6826 | 0.99999 | 0.99999 | 1 | 17945 | 0 | −1.6826 |
| PSMG4 | 4 | 1.43E−05 | 5.07E−05 | 0.007045 | 130 | 3 | −0.7686 | 0.81021 | 0.82919 | 1 | 13741 | 1 | −0.7686 |
| ORAOV1 | 4 | 1.45E−05 | 5.18E−05 | 0.007142 | 131 | 4 | −1.322 | 0.99999 | 0.99998 | 1 | 17944 | 0 | −1.322 |
| ACTR6 | 4 | 1.49E−05 | 5.35E−05 | 0.007263 | 132 | 4 | −1.5981 | 0.99999 | 0.99998 | 1 | 17943 | 0 | −1.5981 |
| PSMB3 | 4 | 1.52E−05 | 5.40E−05 | 0.007263 | 133 | 4 | −2.1912 | 0.99998 | 0.99998 | 1 | 17942 | 0 | −2.1912 |
| DHX33 | 4 | 1.53E−05 | 5.46E−05 | 0.007263 | 134 | 4 | −1.648 | 0.99918 | 0.99916 | 1 | 17538 | 0 | −1.648 |
| EIF2S3 | 4 | 1.55E−05 | 5.46E−05 | 0.007263 | 135 | 4 | −1.7961 | 0.99998 | 0.99998 | 1 | 17941 | 0 | −1.7961 |
| TEN1 | 4 | 1.56E−05 | 5.51E−05 | 0.007263 | 136 | 4 | −1.566 | 0.99993 | 0.99994 | 1 | 17845 | 0 | −1.566 |
| PRELID1 | 4 | 1.57E−05 | 5.51E−05 | 0.007263 | 137 | 3 | −1.6688 | 0.14511 | 0.29584 | 0.949023 | 5557 | 1 | −1.6688 |
| COASY | 4 | 1.59E−05 | 5.84E−05 | 0.007641 | 138 | 4 | −1.582 | 0.99998 | 0.99998 | 1 | 17940 | 0 | −1.582 |
| VHL | 4 | 1.65E−05 | 6.06E−05 | 0.007871 | 139 | 4 | −1.3858 | 0.99998 | 0.99998 | 1 | 17939 | 0 | −1.3858 |
| NDNL2 | 4 | 1.72E−05 | 6.39E−05 | 0.008172 | 140 | 4 | −1.435 | 0.99998 | 0.99998 | 1 | 17938 | 0 | −1.435 |
| ATP6AP1 | 4 | 1.76E−05 | 6.55E−05 | 0.008172 | 141 | 4 | −1.3496 | 0.99998 | 0.99998 | 1 | 17937 | 0 | −1.3496 |
| METAP1 | 4 | 1.76E−05 | 6.55E−05 | 0.008172 | 142 | 4 | −1.0749 | 0.99998 | 0.99998 | 1 | 17936 | 0 | −1.0749 |
| ATIC | 4 | 1.77E−05 | 6.55E−05 | 0.008172 | 143 | 3 | −1.8981 | 0.64995 | 0.75023 | 1 | 12492 | 1 | −1.8981 |
| UTP15 | 4 | 1.78E−05 | 6.55E−05 | 0.008172 | 144 | 4 | −2.8204 | 0.99998 | 0.99998 | 1 | 17935 | 0 | −2.8204 |
| HSP90B1 | 4 | 1.79E−05 | 6.61E−05 | 0.008172 | 145 | 4 | −1.3067 | 0.99998 | 0.99998 | 1 | 17934 | 0 | −1.3067 |
| DHX15 | 4 | 1.79E−05 | 6.61E−05 | 0.008172 | 146 | 4 | −1.2899 | 0.99998 | 0.99998 | 1 | 17933 | 0 | −1.2899 |
| MRP63 | 4 | 1.82E−05 | 6.72E−05 | 0.008251 | 147 | 4 | −1.5761 | 0.99998 | 0.99998 | 1 | 17932 | 0 | −1.5761 |
| TOMM70A | 4 | 1.90E−05 | 7.07E−05 | 0.008614 | 148 | 4 | −1.4334 | 0.99998 | 0.99998 | 1 | 17931 | 0 | −1.4334 |
| DRAP1 | 4 | 1.91E−05 | 7.16E−05 | 0.008614 | 149 | 4 | −1.8359 | 0.99995 | 0.99995 | 1 | 17865 | 0 | −1.8359 |
| RPL36 | 4 | 1.91E−05 | 7.16E−05 | 0.008614 | 150 | 4 | −1.5617 | 0.99998 | 0.99998 | 1 | 17930 | 0 | −1.5617 |
| NOL9 | 4 | 1.96E−05 | 7.32E−05 | 0.008754 | 151 | 4 | −1.4333 | 0.99998 | 0.99998 | 1 | 17929 | 0 | −1.4333 |
| RNGTT | 4 | 1.99E−05 | 7.38E−05 | 0.008761 | 152 | 4 | −2.23 | 0.99998 | 0.99998 | 1 | 17928 | 0 | −2.23 |
| PSMG3 | 4 | 2.04E−05 | 7.54E−05 | 0.008867 | 153 | 4 | −2.4141 | 0.99998 | 0.99998 | 1 | 17927 | 0 | −2.4141 |
| DMAP1 | 4 | 2.05E−05 | 7.60E−05 | 0.008867 | 154 | 4 | −1.5744 | 0.99992 | 0.99993 | 1 | 17835 | 0 | −1.5744 |
| PPP1R2 | 4 | 2.08E−05 | 7.68E−05 | 0.008867 | 155 | 4 | −1.2783 | 0.99998 | 0.99998 | 1 | 17926 | 0 | −1.2783 |
| NOL11 | 4 | 2.09E−05 | 7.71E−05 | 0.008867 | 156 | 4 | −1.5755 | 0.99998 | 0.99998 | 1 | 17925 | 0 | −1.5755 |
| RPL11 | 4 | 2.09E−05 | 7.76E−05 | 0.008867 | 157 | 4 | −2.0256 | 0.99998 | 0.99998 | 1 | 17924 | 0 | −2.0256 |
| CTDNEP1 | 4 | 2.11E−05 | 7.76E−05 | 0.008867 | 158 | 4 | −2.2209 | 0.99998 | 0.99998 | 1 | 17923 | 0 | −2.2209 |
| TPT1 | 4 | 2.13E−05 | 7.82E−05 | 0.008874 | 159 | 4 | −1.2672 | 0.99998 | 0.99998 | 1 | 17922 | 0 | −1.2672 |
| NDUFA13 | 4 | 2.16E−05 | 7.93E−05 | 0.008886 | 160 | 4 | −2.5614 | 0.99998 | 0.99998 | 1 | 17915 | 0 | −2.5614 |
| TIMM44 | 4 | 2.18E−05 | 7.93E−05 | 0.008886 | 161 | 4 | −1.3093 | 0.99998 | 0.99998 | 1 | 17921 | 0 | −1.3093 |
| ISG20L2 | 4 | 2.19E−05 | 7.98E−05 | 0.008893 | 162 | 4 | −1.7604 | 0.99995 | 0.99996 | 1 | 17866 | 0 | −1.7604 |
| PES1 | 4 | 2.21E−05 | 8.20E−05 | 0.009081 | 163 | 4 | −2.1931 | 0.99998 | 0.99998 | 1 | 17920 | 0 | −2.1931 |
| CTCF | 4 | 2.25E−05 | 8.25E−05 | 0.009086 | 164 | 4 | −1.5358 | 0.99998 | 0.99998 | 1 | 17919 | 0 | −1.5358 |
| ERCC2 | 4 | 2.28E−05 | 8.47E−05 | 0.009271 | 165 | 4 | −1.5984 | 0.99994 | 0.99995 | 1 | 17853 | 0 | −1.5984 |
| NCAPH | 4 | 2.31E−05 | 8.75E−05 | 0.009488 | 166 | 4 | −1.1362 | 0.99998 | 0.99998 | 1 | 17918 | 0 | −1.1362 |
| TOP3A | 4 | 2.39E−05 | 8.97E−05 | 0.009488 | 167 | 4 | −1.4901 | 0.99998 | 0.99998 | 1 | 17917 | 0 | −1.4901 |
| MTG2 | 4 | 2.40E−05 | 8.97E−05 | 0.009488 | 168 | 4 | −1.7185 | 0.99992 | 0.99992 | 1 | 17832 | 0 | −1.7185 |
| INTS3 | 4 | 2.41E−05 | 8.99E−05 | 0.009488 | 169 | 4 | −1.2135 | 0.99998 | 0.99998 | 1 | 17916 | 0 | −1.2135 |
| BRF1 | 4 | 2.45E−05 | 9.13E−05 | 0.009488 | 170 | 4 | −1.6094 | 0.99998 | 0.99998 | 1 | 17914 | 0 | −1.6094 |
| PPIL2 | 4 | 2.53E−05 | 9.41E−05 | 0.009488 | 171 | 4 | −1.0274 | 0.99997 | 0.99997 | 1 | 17913 | 0 | −1.0274 |
| PIK3C3 | 4 | 2.54E−05 | 9.41E−05 | 0.009488 | 172 | 4 | −1.693 | 0.99997 | 0.99997 | 1 | 17912 | 0 | −1.693 |
| SRP9 | 4 | 2.54E−05 | 9.41E−05 | 0.009488 | 173 | 4 | −1.6282 | 0.99122 | 0.99133 | 1 | 16983 | 0 | −1.6282 |
| MCTS1 | 4 | 2.55E−05 | 9.41E−05 | 0.009488 | 174 | 4 | −1.3821 | 0.99997 | 0.99997 | 1 | 17911 | 0 | −1.3821 |
| TCOF1 | 4 | 2.55E−05 | 9.41E−05 | 0.009488 | 175 | 4 | −1.5447 | 0.99997 | 0.99997 | 1 | 17910 | 0 | −1.5447 |
| DDB1 | 4 | 2.56E−05 | 9.41E−05 | 0.009488 | 176 | 4 | −1.3482 | 0.99997 | 0.99997 | 1 | 17909 | 0 | −1.3482 |
| UXT | 4 | 2.57E−05 | 9.41E−05 | 0.009488 | 177 | 4 | −2.1137 | 0.99708 | 0.99712 | 1 | 17290 | 0 | −2.1137 |
| MRPL47 | 4 | 2.59E−05 | 9.46E−05 | 0.009488 | 178 | 4 | −1.4143 | 0.99997 | 0.99997 | 1 | 17908 | 0 | −1.4143 |
| COQ6 | 4 | 2.60E−05 | 9.46E−05 | 0.009488 | 179 | 4 | −0.94709 | 0.99997 | 0.99997 | 1 | 17907 | 0 | −0.94709 |
| TUBD1 | 4 | 2.61E−05 | 9.46E−05 | 0.009488 | 180 | 4 | −1.4621 | 0.99962 | 0.99963 | 1 | 17665 | 0 | −1.4621 |
| RPL38 | 4 | 2.74E−05 | 0.00010009 | 0.009983 | 181 | 4 | −1.0644 | 0.99997 | 0.99997 | 1 | 17906 | 0 | −1.0644 |
| HJURP | 4 | 2.84E−05 | 0.00010448 | 0.010363 | 182 | 4 | −0.90685 | 0.99997 | 0.99997 | 1 | 17905 | 0 | −0.90685 |
| MRPL28 | 4 | 2.93E−05 | 0.00010777 | 0.010627 | 183 | 3 | −1.574 | 0.9481 | 0.94802 | 1 | 15891 | 0 | −1.574 |
| NSMCE1 | 4 | 2.98E−05 | 0.00010832 | 0.010627 | 184 | 4 | −1.2423 | 0.99997 | 0.99997 | 1 | 17904 | 0 | −1.2423 |
| NOL10 | 4 | 3.11E−05 | 0.00011161 | 0.010828 | 185 | 4 | −1.6898 | 0.99997 | 0.99997 | 1 | 17903 | 0 | −1.6898 |
| POLR3F | 4 | 3.12E−05 | 0.00011216 | 0.010828 | 186 | 4 | −1.3191 | 0.99997 | 0.99997 | 1 | 17902 | 0 | −1.3191 |
| CCT4 | 4 | 3.12E−05 | 0.00011216 | 0.010828 | 187 | 4 | −2.5971 | 0.99997 | 0.99997 | 1 | 17901 | 0 | −2.5971 |
| ATP5O | 4 | 3.16E−05 | 0.00011435 | 0.010923 | 188 | 4 | −1.9391 | 0.99989 | 0.99989 | 1 | 17807 | 0 | −1.9391 |
| XRCC6 | 4 | 3.17E−05 | 0.00011435 | 0.010923 | 189 | 3 | −1.1921 | 0.95461 | 0.95445 | 1 | 16007 | 0 | −1.1921 |
| CCDC84 | 4 | 3.23E−05 | 0.00011654 | 0.011049 | 190 | 4 | −1.8895 | 0.99997 | 0.99997 | 1 | 17900 | 0 | −1.8895 |
| SPCS2 | 4 | 3.26E−05 | 0.00011764 | 0.011049 | 191 | 4 | −1.7797 | 0.99997 | 0.99997 | 1 | 17899 | 0 | −1.7797 |
| URB1 | 4 | 3.29E−05 | 0.00011874 | 0.011049 | 192 | 4 | −1.3853 | 0.99985 | 0.99986 | 1 | 17776 | 0 | −1.3853 |
| MMS19 | 4 | 3.30E−05 | 0.00011874 | 0.011049 | 193 | 4 | −1.0752 | 0.99552 | 0.9956 | 1 | 17188 | 0 | −1.0752 |
| TAF1B | 4 | 3.31E−05 | 0.00011874 | 0.011049 | 194 | 4 | −1.2269 | 0.99997 | 0.99997 | 1 | 17898 | 0 | −1.2269 |
| DNAJA3 | 4 | 3.35E−05 | 0.00012093 | 0.011176 | 195 | 4 | −1.8323 | 0.99997 | 0.99997 | 1 | 17897 | 0 | −1.8323 |
| POLR3B | 4 | 3.37E−05 | 0.00012148 | 0.011176 | 196 | 4 | −2.4368 | 0.99997 | 0.99997 | 1 | 17896 | 0 | −2.4368 |
| TAF3 | 4 | 3.39E−05 | 0.00012203 | 0.011176 | 197 | 4 | −0.95126 | 0.99711 | 0.99714 | 1 | 17293 | 0 | −0.95126 |
| NFS1 | 4 | 3.40E−05 | 0.00012258 | 0.011176 | 198 | 3 | −1.6063 | 0.4684 | 0.62372 | 1 | 10577 | 1 | −1.6063 |
| WBSCR16 | 4 | 3.48E−05 | 0.00012751 | 0.011444 | 199 | 4 | −1.2883 | 0.99997 | 0.99997 | 1 | 17895 | 0 | −1.2883 |
| RPN1 | 4 | 3.51E−05 | 0.00012971 | 0.011444 | 200 | 4 | −1.0861 | 0.99996 | 0.99997 | 1 | 17894 | 0 | −1.0861 |
| MIPEP | 4 | 3.52E−05 | 0.00013025 | 0.011444 | 201 | 4 | −1.3452 | 0.99996 | 0.99997 | 1 | 17893 | 0 | −1.3452 |
| BANF1 | 4 | 3.53E−05 | 0.00013025 | 0.011444 | 202 | 4 | −1.7906 | 0.99976 | 0.99976 | 1 | 17728 | 0 | −1.7906 |
| GTF2H1 | 4 | 3.57E−05 | 0.0001308 | 0.011444 | 203 | 4 | −0.84807 | 0.99996 | 0.99996 | 1 | 17892 | 0 | −0.84807 |
| IARS | 4 | 3.57E−05 | 0.00013135 | 0.011444 | 204 | 4 | −1.4993 | 0.99985 | 0.99986 | 1 | 17775 | 0 | −1.4993 |
| OGDH | 4 | 3.61E−05 | 0.0001319 | 0.011444 | 205 | 4 | −1.3055 | 0.99996 | 0.99996 | 1 | 17891 | 0 | −1.3055 |
| CDC37 | 4 | 3.63E−05 | 0.00013245 | 0.011444 | 206 | 4 | −1.7045 | 0.99996 | 0.99996 | 1 | 17890 | 0 | −1.7045 |
| AURKAIP1 | 4 | 3.64E−05 | 0.000133 | 0.011444 | 207 | 4 | −1.3848 | 0.99996 | 0.99996 | 1 | 17889 | 0 | −1.3848 |
| LUC7L3 | 4 | 3.66E−05 | 0.00013355 | 0.011444 | 208 | 4 | −1.284 | 0.99996 | 0.99996 | 1 | 17888 | 0 | −1.284 |
| PFAS | 4 | 3.66E−05 | 0.00013409 | 0.011444 | 209 | 4 | −1.6487 | 0.99964 | 0.99966 | 1 | 17679 | 0 | −1.6487 |
| EIF1AD | 4 | 3.67E−05 | 0.00013409 | 0.011444 | 210 | 4 | −1.3503 | 0.99996 | 0.99996 | 1 | 17887 | 0 | −1.3503 |
| RIOK1 | 4 | 3.67E−05 | 0.00013409 | 0.011444 | 211 | 4 | −1.9773 | 0.99996 | 0.99996 | 1 | 17886 | 0 | −1.9773 |
| SRP14 | 4 | 3.68E−05 | 0.00013464 | 0.011444 | 212 | 4 | −1.4386 | 0.99996 | 0.99996 | 1 | 17885 | 0 | −1.4386 |
| UQCRFS1 | 4 | 3.71E−05 | 0.00013629 | 0.011444 | 213 | 4 | −3.0912 | 0.99996 | 0.99996 | 1 | 17884 | 0 | −3.0912 |
| SPATA5L1 | 4 | 3.72E−05 | 0.00013629 | 0.011444 | 214 | 3 | −1.6812 | 0.67725 | 0.7613 | 1 | 12666 | 1 | −1.6812 |
| MRPS16 | 4 | 3.74E−05 | 0.00013793 | 0.011468 | 215 | 4 | −1.3711 | 0.99996 | 0.99996 | 1 | 17883 | 0 | −1.3711 |
| EXOSC10 | 4 | 3.75E−05 | 0.00013793 | 0.011468 | 216 | 4 | −1.1568 | 0.99996 | 0.99996 | 1 | 17882 | 0 | −1.1568 |
| TOP2A | 4 | 3.75E−05 | 0.00013848 | 0.011468 | 217 | 4 | −1.7788 | 0.99996 | 0.99996 | 1 | 17881 | 0 | −1.7788 |
| CTC1 | 4 | 3.87E−05 | 0.00014397 | 0.011868 | 218 | 4 | −1.0186 | 0.99996 | 0.99996 | 1 | 17880 | 0 | −1.0186 |
| PRMT1 | 4 | 4.11E−05 | 0.00015274 | 0.012534 | 219 | 4 | −1.2984 | 0.99996 | 0.99996 | 1 | 17879 | 0 | −1.2984 |
| DDX59 | 3 | 4.15E−05 | 0.00013574 | 0.011444 | 220 | 3 | −1.7396 | 0.99996 | 0.99996 | 1 | 17878 | 0 | −1.7396 |
| ILF2 | 4 | 4.17E−05 | 0.00015548 | 0.012701 | 221 | 4 | −1.2814 | 0.99996 | 0.99996 | 1 | 17877 | 0 | −1.2814 |
| FBXW11 | 4 | 4.24E−05 | 0.00015822 | 0.012867 | 222 | 4 | −1.2411 | 0.99996 | 0.99996 | 1 | 17876 | 0 | −1.2411 |
| MARS | 4 | 4.32E−05 | 0.00016097 | 0.012902 | 223 | 4 | −1.3073 | 0.99996 | 0.99996 | 1 | 17875 | 0 | −1.3073 |
| CHTF8 | 4 | 4.33E−05 | 0.00016097 | 0.012902 | 224 | 4 | −1.4985 | 0.99996 | 0.99996 | 1 | 17874 | 0 | −1.4985 |
| TOE1 | 4 | 4.37E−05 | 0.00016152 | 0.012902 | 225 | 4 | −1.4137 | 0.99792 | 0.99796 | 1 | 17368 | 0 | −1.4137 |
| PMM2 | 4 | 4.37E−05 | 0.00016152 | 0.012902 | 226 | 4 | −1.576 | 0.99996 | 0.99996 | 1 | 17873 | 0 | −1.576 |
| ALDOA | 4 | 4.47E−05 | 0.00016535 | 0.013079 | 227 | 4 | −1.7059 | 0.99996 | 0.99996 | 1 | 17872 | 0 | −1.7059 |
| MRPL41 | 4 | 4.50E−05 | 0.00016535 | 0.013079 | 228 | 4 | −1.3252 | 0.99995 | 0.99996 | 1 | 17871 | 0 | −1.3252 |
| GEMIN8 | 4 | 4.52E−05 | 0.0001659 | 0.013079 | 229 | 4 | −1.3382 | 0.99995 | 0.99996 | 1 | 17870 | 0 | −1.3382 |
| CDC23 | 4 | 4.68E−05 | 0.00017194 | 0.013422 | 230 | 4 | −2.2073 | 0.99995 | 0.99996 | 1 | 17869 | 0 | −2.2073 |
| MTX1 | 4 | 4.70E−05 | 0.00017248 | 0.013422 | 231 | 4 | −1.0803 | 0.99995 | 0.99996 | 1 | 17868 | 0 | −1.0803 |
| TFAM | 4 | 4.84E−05 | 0.00018071 | 0.013984 | 232 | 4 | −1.4129 | 0.99945 | 0.99947 | 1 | 17605 | 0 | −1.4129 |
| RBBP4 | 4 | 4.84E−05 | 0.00018126 | 0.013984 | 233 | 4 | −1.1574 | 0.99995 | 0.99996 | 1 | 17867 | 0 | −1.1574 |
| MRPL39 | 4 | 5.01E−05 | 0.0001862 | 0.014304 | 234 | 4 | −1.5499 | 0.99697 | 0.99701 | 1 | 17285 | 0 | −1.5499 |
| SLC7A6OS | 4 | 5.08E−05 | 0.00018839 | 0.014411 | 235 | 4 | −2.7784 | 0.99995 | 0.99995 | 1 | 17864 | 0 | −2.7784 |
| ATP6V0C | 4 | 5.13E−05 | 0.00019058 | 0.014498 | 236 | 4 | −1.9582 | 0.99995 | 0.99995 | 1 | 17863 | 0 | −1.9582 |
| ANKRD49 | 4 | 5.14E−05 | 0.00019113 | 0.014498 | 237 | 4 | −1.5715 | 0.99979 | 0.99979 | 1 | 17743 | 0 | −1.5715 |
| SARS2 | 4 | 5.19E−05 | 0.00019278 | 0.014561 | 238 | 4 | −1.6139 | 0.9999 | 0.99991 | 1 | 17821 | 0 | −1.6139 |
| POLR1B | 4 | 5.32E−05 | 0.00019607 | 0.014708 | 239 | 4 | −1.7714 | 0.99995 | 0.99995 | 1 | 17862 | 0 | −1.7714 |
| CD3EAP | 4 | 5.36E−05 | 0.00019662 | 0.014708 | 240 | 4 | −1.0176 | 0.99521 | 0.99529 | 1 | 17173 | 0 | −1.0176 |
| NELFA | 4 | 5.37E−05 | 0.00019716 | 0.014708 | 241 | 4 | −1.5096 | 0.99956 | 0.99958 | 1 | 17638 | 0 | −1.5096 |
| HAUS1 | 3 | 5.46E−05 | 0.00017194 | 0.013422 | 242 | 3 | −1.2521 | 0.99995 | 0.99995 | 1 | 17861 | 0 | −1.2521 |
| VPS16 | 4 | 5.54E−05 | 0.00020375 | 0.015137 | 243 | 4 | −1.3667 | 0.99994 | 0.99995 | 1 | 17860 | 0 | −1.3667 |
| ORC2 | 4 | 5.58E−05 | 0.00020484 | 0.015156 | 244 | 4 | −1.463 | 0.98885 | 0.98899 | 1 | 16874 | 0 | −1.463 |
| TRAPPC3 | 4 | 5.62E−05 | 0.00020594 | 0.015175 | 245 | 4 | −1.3019 | 0.99994 | 0.99995 | 1 | 17859 | 0 | −1.3019 |
| MRPL3 | 4 | 5.64E−05 | 0.00020813 | 0.015274 | 246 | 4 | −1.2455 | 0.9985 | 0.99852 | 1 | 17431 | 0 | −1.2455 |
| CDIPT | 4 | 5.67E−05 | 0.00020978 | 0.015333 | 247 | 4 | −2.141 | 0.99952 | 0.99955 | 1 | 17623 | 0 | −2.141 |
| MGEA5 | 4 | 5.75E−05 | 0.00021142 | 0.01539 | 248 | 4 | −0.73113 | 0.99994 | 0.99995 | 1 | 17858 | 0 | −0.73113 |
| YAE1D1 | 4 | 5.82E−05 | 0.00021417 | 0.015483 | 249 | 4 | −1.2102 | 0.9782 | 0.97829 | 1 | 16528 | 0 | −1.2102 |
| YARS | 4 | 5.85E−05 | 0.00021526 | 0.015483 | 250 | 4 | −1.6791 | 0.99994 | 0.99995 | 1 | 17857 | 0 | −1.6791 |
| TUBG1 | 4 | 5.87E−05 | 0.00021691 | 0.01549 | 251 | 4 | −1.3919 | 0.99994 | 0.99995 | 1 | 17856 | 0 | −1.3919 |
| RRP9 | 4 | 5.91E−05 | 0.000218 | 0.01549 | 252 | 3 | −1.5443 | 0.96702 | 0.96693 | 1 | 16263 | 0 | −1.5443 |
| CARS2 | 4 | 5.94E−05 | 0.00021855 | 0.01549 | 253 | 4 | −1.1901 | 0.99994 | 0.99995 | 1 | 17855 | 0 | −1.1901 |
| NUP50 | 4 | 5.95E−05 | 0.0002191 | 0.01549 | 254 | 4 | −1.2224 | 0.99994 | 0.99995 | 1 | 17854 | 0 | −1.2224 |
| RBM17 | 4 | 6.00E−05 | 0.00021965 | 0.01549 | 255 | 4 | −2.0901 | 0.99427 | 0.99438 | 1 | 17138 | 0 | −2.0901 |
| DHODH | 4 | 6.03E−05 | 0.0002213 | 0.015545 | 256 | 4 | −1.0687 | 0.99924 | 0.99924 | 1 | 17549 | 0 | −1.0687 |
| MAD2L2 | 4 | 6.05E−05 | 0.00022239 | 0.015561 | 257 | 4 | −1.0915 | 0.99954 | 0.99957 | 1 | 17631 | 0 | −1.0915 |
| PPP2R4 | 4 | 6.10E−05 | 0.00022513 | 0.015692 | 258 | 4 | −1.4737 | 0.99994 | 0.99995 | 1 | 17852 | 0 | −1.4737 |
| ABCB7 | 4 | 6.14E−05 | 0.00022623 | 0.015708 | 259 | 4 | −1.4324 | 0.99994 | 0.99995 | 1 | 17851 | 0 | −1.4324 |
| NHP2 | 4 | 6.16E−05 | 0.00022733 | 0.015724 | 260 | 4 | −1.7728 | 0.99994 | 0.99995 | 1 | 17850 | 0 | −1.7728 |
| AP2B1 | 4 | 6.29E−05 | 0.00023446 | 0.016155 | 261 | 4 | −0.9467 | 0.99994 | 0.99995 | 1 | 17849 | 0 | −0.9467 |
| RPS16 | 4 | 6.36E−05 | 0.00023665 | 0.016159 | 262 | 4 | −1.6096 | 0.99994 | 0.99994 | 1 | 17848 | 0 | −1.6096 |
| OIP5 | 4 | 6.36E−05 | 0.00023665 | 0.016159 | 263 | 4 | −1.9895 | 0.99921 | 0.9992 | 1 | 17544 | 0 | −1.9895 |
| RPP21 | 4 | 6.37E−05 | 0.0002372 | 0.016159 | 264 | 4 | −1.9962 | 0.9999 | 0.9999 | 1 | 17815 | 0 | −1.9962 |
| UGP2 | 4 | 6.46E−05 | 0.00024268 | 0.016471 | 265 | 4 | −1.3838 | 0.9996 | 0.99962 | 1 | 17651 | 0 | −1.3838 |
| MRPL45 | 4 | 6.48E−05 | 0.00024378 | 0.016483 | 266 | 4 | −1.7926 | 0.99976 | 0.99977 | 1 | 17732 | 0 | −1.7926 |
| DPAGT1 | 4 | 6.52E−05 | 0.00024543 | 0.016508 | 267 | 4 | −1.2661 | 0.99993 | 0.99994 | 1 | 17847 | 0 | −1.2661 |
| NHLRC2 | 4 | 6.54E−05 | 0.00024598 | 0.016508 | 268 | 4 | −0.99199 | 0.99993 | 0.99994 | 1 | 17846 | 0 | −0.99199 |
| PYROXD1 | 4 | 6.73E−05 | 0.00025201 | 0.01685 | 269 | 3 | −2.4967 | 0.96937 | 0.96927 | 1 | 16305 | 0 | −2.4967 |
| UBA52 | 3 | 6.85E−05 | 0.00021471 | 0.015483 | 270 | 3 | −2.8242 | 0.99993 | 0.99993 | 1 | 17844 | 0 | −2.8242 |
| ORC6 | 4 | 6.93E−05 | 0.00025914 | 0.017263 | 271 | 4 | −1.698 | 0.99992 | 0.99993 | 1 | 17837 | 0 | −1.698 |
| GART | 4 | 7.04E−05 | 0.00026188 | 0.017381 | 272 | 3 | −1.9946 | 0.50004 | 0.65114 | 1 | 10972 | 1 | −1.9946 |
| TIMM10 | 4 | 7.05E−05 | 0.00026298 | 0.01739 | 273 | 2 | −0.88764 | 0.83889 | 0.8481 | 1 | 14080 | 1 | −0.88764 |
| PDAP1 | 4 | 7.23E−05 | 0.00026682 | 0.017398 | 274 | 4 | −1.3936 | 0.99993 | 0.99994 | 1 | 17842 | 0 | −1.3936 |
| COA5 | 4 | 7.24E−05 | 0.00026682 | 0.017398 | 275 | 4 | −1.2586 | 0.99147 | 0.99159 | 1 | 16997 | 0 | −1.2586 |
| MTOR | 4 | 7.26E−05 | 0.00026736 | 0.017398 | 276 | 4 | −1.203 | 0.99912 | 0.99911 | 1 | 17523 | 0 | −1.203 |
| HARS2 | 4 | 7.29E−05 | 0.00026736 | 0.017398 | 277 | 4 | −1.5834 | 0.99993 | 0.99994 | 1 | 17841 | 0 | −1.5834 |
| PELP1 | 4 | 7.31E−05 | 0.00026791 | 0.017398 | 278 | 4 | −1.827 | 0.99954 | 0.99956 | 1 | 17629 | 0 | −1.827 |
| GTF2H4 | 4 | 7.48E−05 | 0.00027669 | 0.017903 | 279 | 4 | −1.2455 | 0.99993 | 0.99993 | 1 | 17840 | 0 | −1.2455 |
| GNB2L1 | 4 | 7.49E−05 | 0.00027888 | 0.017981 | 280 | 4 | −1.1995 | 0.99993 | 0.99993 | 1 | 17839 | 0 | −1.1995 |
| GOT2 | 4 | 7.61E−05 | 0.00028272 | 0.018164 | 281 | 4 | −0.92182 | 0.99992 | 0.99993 | 1 | 17838 | 0 | −0.92182 |
| RPRD1B | 4 | 7.65E−05 | 0.00028382 | 0.018169 | 282 | 3 | −1.6447 | 0.94351 | 0.94336 | 1 | 15796 | 0 | −1.6447 |
| KIDINS220 | 4 | 7.89E−05 | 0.00029534 | 0.01884 | 283 | 4 | −1.5321 | 0.99992 | 0.99993 | 1 | 17836 | 0 | −1.5321 |
| MRPL37 | 4 | 7.95E−05 | 0.00029753 | 0.018881 | 284 | 4 | −1.5113 | 0.99826 | 0.99829 | 1 | 17404 | 0 | −1.5113 |
| NARS | 4 | 7.97E−05 | 0.00029808 | 0.018881 | 285 | 4 | −1.3406 | 0.99992 | 0.99993 | 1 | 17834 | 0 | −1.3406 |
| TSC1 | 4 | 8.00E−05 | 0.00029972 | 0.018919 | 286 | 4 | −1.5691 | 0.99986 | 0.99987 | 1 | 17786 | 0 | −1.5691 |
| POLR3C | 4 | 8.12E−05 | 0.00030411 | 0.019129 | 287 | 4 | −1.6951 | 0.99986 | 0.99987 | 1 | 17784 | 0 | −1.6951 |
| SEC63 | 4 | 8.15E−05 | 0.00030576 | 0.019166 | 288 | 4 | −1.2981 | 0.99964 | 0.99965 | 1 | 17677 | 0 | −1.2981 |
| QRSL1 | 4 | 8.31E−05 | 0.00030959 | 0.019244 | 289 | 4 | −1.6791 | 0.99284 | 0.99295 | 1 | 17071 | 0 | −1.6791 |
| RPIA | 4 | 8.32E−05 | 0.00031014 | 0.019244 | 290 | 3 | −1.8545 | 0.37108 | 0.53926 | 1 | 9348 | 1 | −1.8545 |
| THOC7 | 4 | 8.36E−05 | 0.00031179 | 0.019244 | 291 | 4 | −1.6485 | 0.99776 | 0.99779 | 1 | 17352 | 0 | −1.6485 |
| BUB3 | 4 | 8.36E−05 | 0.00031179 | 0.019244 | 292 | 4 | −1.2091 | 0.99992 | 0.99992 | 1 | 17833 | 0 | −1.2091 |
| CENPW | 4 | 8.40E−05 | 0.00031234 | 0.019244 | 293 | 4 | −2.0777 | 0.99992 | 0.99992 | 1 | 17831 | 0 | −2.0777 |
| STIP1 | 4 | 8.66E−05 | 0.00031892 | 0.019518 | 294 | 4 | −1.27 | 0.99991 | 0.99992 | 1 | 17830 | 0 | −1.27 |
| SDHC | 4 | 8.72E−05 | 0.00032001 | 0.019518 | 295 | 4 | −1.3889 | 0.99869 | 0.9987 | 1 | 17450 | 0 | −1.3889 |
| RIOK2 | 4 | 8.73E−05 | 0.00032001 | 0.019518 | 296 | 4 | −1.4882 | 0.99991 | 0.99992 | 1 | 17829 | 0 | −1.4882 |
| DDX56 | 4 | 8.80E−05 | 0.00032276 | 0.019619 | 297 | 4 | −1.3394 | 0.99991 | 0.99992 | 1 | 17828 | 0 | −1.3394 |
| MRPS18C | 4 | 8.89E−05 | 0.00032605 | 0.019719 | 298 | 3 | −1.1186 | 0.94737 | 0.94728 | 1 | 15873 | 0 | −1.1186 |
| RPN2 | 4 | 8.89E−05 | 0.0003266 | 0.019719 | 299 | 4 | −1.1248 | 0.99553 | 0.99562 | 1 | 17191 | 0 | −1.1248 |
| CENPM | 4 | 9.13E−05 | 0.00033647 | 0.02018 | 300 | 4 | −1.2757 | 0.99991 | 0.99992 | 1 | 17826 | 0 | −1.2757 |
| CIT | 4 | 9.14E−05 | 0.00033647 | 0.02018 | 301 | 4 | −2.1726 | 0.99985 | 0.99986 | 1 | 17778 | 0 | −2.1726 |
| EIF4E | 4 | 9.18E−05 | 0.00033921 | 0.020277 | 302 | 3 | −1.5193 | 0.81307 | 0.83101 | 1 | 13770 | 1 | −1.5193 |
| MPHOSPH10 | 4 | 9.34E−05 | 0.0003436 | 0.020437 | 303 | 4 | −2.0951 | 0.99991 | 0.99991 | 1 | 17825 | 0 | −2.0951 |
| DDX46 | 4 | 9.36E−05 | 0.00034415 | 0.020437 | 304 | 4 | −2.3455 | 0.99991 | 0.99991 | 1 | 17824 | 0 | −2.3455 |
| UBE2S | 4 | 9.43E−05 | 0.00034744 | 0.020565 | 305 | 4 | −0.97481 | 0.99991 | 0.99991 | 1 | 17823 | 0 | −0.97481 |
| EIF3E | 4 | 9.54E−05 | 0.00035237 | 0.020786 | 306 | 4 | −1.5713 | 0.99973 | 0.99974 | 1 | 17711 | 0 | −1.5713 |
| IMPDH2 | 4 | 9.55E−05 | 0.00035347 | 0.020786 | 307 | 4 | −1.0235 | 0.9999 | 0.99991 | 1 | 17822 | 0 | −1.0235 |
| SOD2 | 4 | 9.59E−05 | 0.00035511 | 0.020815 | 308 | 3 | −0.93987 | 0.89255 | 0.89226 | 1 | 14830 | 1 | −0.93987 |
| UBE2M | 4 | 9.69E−05 | 0.00036005 | 0.020876 | 309 | 4 | −1.4821 | 0.99973 | 0.99974 | 1 | 17712 | 0 | −1.4821 |
| EXOC4 | 4 | 9.71E−05 | 0.00036005 | 0.020876 | 310 | 3 | −1.4493 | 0.82162 | 0.83644 | 1 | 13882 | 1 | −1.4493 |
| SACM1L | 4 | 9.75E−05 | 0.00036389 | 0.020876 | 311 | 4 | −1.2363 | 0.9999 | 0.99991 | 1 | 17820 | 0 | −1.2363 |
| CIAO1 | 4 | 9.77E−05 | 0.00036444 | 0.020876 | 312 | 4 | −1.3908 | 0.9999 | 0.99991 | 1 | 17819 | 0 | −1.3908 |
| CCNK | 4 | 9.83E−05 | 0.00036608 | 0.020876 | 313 | 4 | −1.3783 | 0.9999 | 0.99991 | 1 | 17818 | 0 | −1.3783 |
| EPRS | 4 | 9.88E−05 | 0.00036663 | 0.020876 | 314 | 4 | −0.90649 | 0.9999 | 0.99991 | 1 | 17817 | 0 | −0.90649 |
| COG1 | 4 | 9.88E−05 | 0.00036718 | 0.020876 | 315 | 4 | −0.79771 | 0.9999 | 0.99991 | 1 | 17816 | 0 | −0.79771 |
| DDX55 | 4 | 9.89E−05 | 0.00036718 | 0.020876 | 316 | 4 | −1.7633 | 0.99195 | 0.99207 | 1 | 17022 | 0 | −1.7633 |
| COQ4 | 4 | 9.92E−05 | 0.00036773 | 0.020876 | 317 | 4 | −1.9034 | 0.98936 | 0.98948 | 1 | 16892 | 0 | −1.9034 |
| GATC | 4 | 9.93E−05 | 0.00036773 | 0.020876 | 318 | 3 | −1.0106 | 0.93978 | 0.93966 | 1 | 15731 | 0 | −1.0106 |
| ECSIT | 4 | 0.00010099 | 0.00037376 | 0.021152 | 319 | 3 | −1.1098 | 0.84574 | 0.85304 | 1 | 14153 | 1 | −1.1098 |
| TFB2M | 4 | 0.00010363 | 0.00038473 | 0.021705 | 320 | 4 | −1.3146 | 0.99634 | 0.99641 | 1 | 17236 | 0 | −1.3146 |
| TSC2 | 4 | 0.0001051 | 0.00038912 | 0.021816 | 321 | 4 | −1.8222 | 0.99824 | 0.99827 | 1 | 17401 | 0 | −1.8222 |
| COX6B1 | 4 | 0.00010518 | 0.00038912 | 0.021816 | 322 | 3 | −0.89953 | 0.876 | 0.87712 | 1 | 14557 | 1 | −0.89953 |
| ATP1A1 | 4 | 0.00010823 | 0.00040173 | 0.022415 | 323 | 4 | −1.1615 | 0.99987 | 0.99988 | 1 | 17797 | 0 | −1.1615 |
| PMPCA | 4 | 0.00010868 | 0.00040228 | 0.022415 | 324 | 3 | −1.9866 | 0.90225 | 0.90187 | 1 | 15021 | 0 | −1.9866 |
| THOC6 | 4 | 0.00011049 | 0.00040722 | 0.02262 | 325 | 4 | −0.85665 | 0.99989 | 0.99989 | 1 | 17814 | 0 | −0.85665 |
| ZBTB17 | 4 | 0.00011128 | 0.00040996 | 0.022702 | 326 | 4 | −1.0572 | 0.99989 | 0.99989 | 1 | 17813 | 0 | −1.0572 |
| CCT7 | 4 | 0.00011234 | 0.00041599 | 0.022926 | 327 | 4 | −2.2631 | 0.99975 | 0.99976 | 1 | 17724 | 0 | −2.2631 |
| WDR61 | 4 | 0.00011239 | 0.00041654 | 0.022926 | 328 | 4 | −1.0448 | 0.99989 | 0.99989 | 1 | 17812 | 0 | −1.0448 |
| GFM2 | 4 | 0.00011357 | 0.00042257 | 0.023008 | 329 | 3 | −1.2265 | 0.80933 | 0.82865 | 1 | 13734 | 1 | −1.2265 |
| RPA1 | 4 | 0.00011369 | 0.00042257 | 0.023008 | 330 | 4 | −1.6611 | 0.99989 | 0.99989 | 1 | 17811 | 0 | −1.6611 |
| TSEN54 | 4 | 0.00011373 | 0.00042257 | 0.023008 | 331 | 4 | −1.4308 | 0.99821 | 0.99824 | 1 | 17397 | 0 | −1.4308 |
| CIAPIN1 | 4 | 0.00011393 | 0.00042312 | 0.023008 | 332 | 4 | −1.0569 | 0.99989 | 0.99989 | 1 | 17810 | 0 | −1.0569 |
| DTL | 4 | 0.00011425 | 0.00042696 | 0.023147 | 333 | 4 | −1.4574 | 0.99989 | 0.99989 | 1 | 17809 | 0 | −1.4574 |
| RAB10 | 4 | 0.00011481 | 0.00043025 | 0.023255 | 334 | 4 | −1.4273 | 0.99989 | 0.99989 | 1 | 17808 | 0 | −1.4273 |
| TOMM40 | 4 | 0.00011529 | 0.0004319 | 0.023275 | 335 | 4 | −1.573 | 0.99954 | 0.99956 | 1 | 17627 | 0 | −1.573 |
| FOXM1 | 4 | 0.00011563 | 0.00043409 | 0.023313 | 336 | 4 | −0.81311 | 0.99988 | 0.99989 | 1 | 17806 | 0 | −0.81311 |
| MRPS18B | 4 | 0.00011602 | 0.00043519 | 0.023313 | 337 | 4 | −1.7006 | 0.99653 | 0.99658 | 1 | 17250 | 0 | −1.7006 |
| NDUFA11 | 4 | 0.00011804 | 0.00044396 | 0.023713 | 338 | 4 | −1.1284 | 0.99988 | 0.99989 | 1 | 17805 | 0 | −1.1284 |
| COA3 | 4 | 0.00011893 | 0.00045054 | 0.023993 | 339 | 3 | −1.5482 | 0.96712 | 0.96703 | 1 | 16265 | 0 | −1.5482 |
| ATP5F1 | 4 | 0.00012094 | 0.00045932 | 0.024317 | 340 | 4 | −1.0899 | 0.99988 | 0.99988 | 1 | 17804 | 0 | −1.0899 |
| HAUS8 | 4 | 0.0001214 | 0.00046151 | 0.024362 | 341 | 4 | −1.3974 | 0.99988 | 0.99988 | 1 | 17803 | 0 | −1.3974 |
| YRDC | 4 | 0.00012396 | 0.00046974 | 0.024566 | 342 | 4 | −1.4554 | 0.99988 | 0.99988 | 1 | 17802 | 0 | −1.4554 |
| WBSCR22 | 4 | 0.00012412 | 0.00047029 | 0.024566 | 343 | 3 | −1.7695 | 0.9617 | 0.96159 | 1 | 16140 | 0 | −1.7695 |
| FARS2 | 4 | 0.00012431 | 0.00047084 | 0.024566 | 344 | 4 | −1.6992 | 0.99943 | 0.99945 | 1 | 17595 | 0 | −1.6992 |
| CTPS1 | 4 | 0.00012496 | 0.00047577 | 0.024724 | 345 | 4 | −1.5858 | 0.99988 | 0.99988 | 1 | 17800 | 0 | −1.5858 |
| GPN3 | 4 | 0.00012544 | 0.00047742 | 0.024724 | 346 | 4 | −1.3885 | 0.99954 | 0.99957 | 1 | 17630 | 0 | −1.3885 |
| SNUPN | 4 | 0.00012569 | 0.00047797 | 0.024724 | 347 | 4 | −1.8808 | 0.99987 | 0.99988 | 1 | 17799 | 0 | −1.8808 |
| TRAIP | 4 | 0.00012778 | 0.00048564 | 0.025035 | 348 | 4 | −1.4132 | 0.99461 | 0.99472 | 1 | 17149 | 0 | −1.4132 |
| WDR25 | 4 | 0.00012826 | 0.00048674 | 0.025035 | 349 | 4 | −1.3438 | 0.99987 | 0.99988 | 1 | 17798 | 0 | −1.3438 |
| PARS2 | 4 | 0.00012874 | 0.00049003 | 0.025132 | 350 | 4 | −1.7845 | 0.99987 | 0.99988 | 1 | 17796 | 0 | −1.7845 |
| ELP4 | 4 | 0.00013288 | 0.00051087 | 0.026127 | 351 | 4 | −0.90171 | 0.99987 | 0.99987 | 1 | 17795 | 0 | −0.90171 |
| TIMM22 | 4 | 0.00013534 | 0.00052074 | 0.02638 | 352 | 4 | −0.90815 | 0.99986 | 0.99987 | 1 | 17794 | 0 | −0.90815 |
| BIRC6 | 4 | 0.00013547 | 0.00052129 | 0.02638 | 353 | 3 | −1.5274 | 0.9681 | 0.96799 | 1 | 16280 | 0 | −1.5274 |
| CTDP1 | 4 | 0.00013556 | 0.00052184 | 0.02638 | 354 | 4 | −1.0547 | 0.99986 | 0.99987 | 1 | 17793 | 0 | −1.0547 |
| EXOSC7 | 4 | 0.00013641 | 0.00052403 | 0.02638 | 355 | 4 | −1.5982 | 0.99986 | 0.99987 | 1 | 17792 | 0 | −1.5982 |
| ALG2 | 4 | 0.0001367 | 0.00052458 | 0.02638 | 356 | 4 | −1.5596 | 0.99986 | 0.99987 | 1 | 17789 | 0 | −1.5596 |
| APEX2 | 4 | 0.00013691 | 0.00052458 | 0.02638 | 357 | 4 | −1.0917 | 0.99986 | 0.99987 | 1 | 17791 | 0 | −1.0917 |
| CCNA2 | 4 | 0.00013784 | 0.00052623 | 0.026389 | 358 | 4 | −1.4742 | 0.99986 | 0.99987 | 1 | 17790 | 0 | −1.4742 |
| MRPS12 | 4 | 0.00013864 | 0.00052787 | 0.026398 | 359 | 4 | −1.0557 | 0.99732 | 0.99735 | 1 | 17311 | 0 | −1.0557 |
| TOMM20 | 4 | 0.00013899 | 0.00052952 | 0.026407 | 360 | 3 | −1.083 | 0.9624 | 0.9623 | 1 | 16157 | 0 | −1.083 |
| EIF2B3 | 4 | 0.00013988 | 0.00053336 | 0.02647 | 361 | 4 | −1.0396 | 0.99787 | 0.99791 | 1 | 17363 | 0 | −1.0396 |
| CMPK1 | 4 | 0.00014059 | 0.00053445 | 0.02647 | 362 | 3 | −1.6524 | 0.92046 | 0.92025 | 1 | 15343 | 0 | −1.6524 |
| GFER | 4 | 0.00014091 | 0.000536 | 0.02647 | 363 | 3 | −1.0451 | 0.7075 | 0.77459 | 1 | 12876 | 1 | −1.0451 |
| CHORDC1 | 4 | 0.00014112 | 0.00053665 | 0.02647 | 364 | 4 | −1.4593 | 0.99698 | 0.99702 | 1 | 17286 | 0 | −1.4593 |
| XRCC2 | 4 | 0.00014167 | 0.00053994 | 0.026515 | 365 | 3 | −0.94862 | 0.94138 | 0.94124 | 1 | 15762 | 0 | −0.94862 |
| CFDP1 | 4 | 0.00014183 | 0.00054049 | 0.026515 | 366 | 4 | −1.4382 | 0.99986 | 0.99987 | 1 | 17788 | 0 | −1.4382 |
| DHDDS | 4 | 0.00014205 | 0.00054213 | 0.026523 | 367 | 4 | −1.2925 | 0.99986 | 0.99987 | 1 | 17787 | 0 | −1.2925 |
| MCM3 | 4 | 0.0001433 | 0.00054707 | 0.026621 | 368 | 4 | −1.1372 | 0.99986 | 0.99987 | 1 | 17785 | 0 | −1.1372 |
| C10orf2 | 4 | 0.00014347 | 0.00054707 | 0.026621 | 369 | 3 | −1.4685 | 0.96457 | 0.96448 | 1 | 16208 | 0 | −1.4685 |
| SAE1 | 4 | 0.00014471 | 0.00055201 | 0.026789 | 370 | 4 | −1.2272 | 0.99986 | 0.99987 | 1 | 17783 | 0 | −1.2272 |
| LETM1 | 3 | 0.000145 | 0.00045658 | 0.024243 | 371 | 3 | −1.3556 | 0.99985 | 0.99985 | 1 | 17782 | 0 | −1.3556 |
| PCBP1 | 4 | 0.00014659 | 0.00055859 | 0.026827 | 372 | 4 | −1.0432 | 0.99985 | 0.99986 | 1 | 17781 | 0 | −1.0432 |
| DNAJC17 | 4 | 0.00014666 | 0.00055913 | 0.026827 | 373 | 4 | −1.0539 | 0.99985 | 0.99986 | 1 | 17780 | 0 | −1.0539 |
| TAF10 | 4 | 0.00014675 | 0.00055913 | 0.026827 | 374 | 3 | −1.2192 | 0.06773 | 0.16625 | 0.8674 | 3408 | 1 | −1.2192 |
| SNAPC4 | 4 | 0.00014694 | 0.00055913 | 0.026827 | 375 | 4 | −1.426 | 0.99289 | 0.99301 | 1 | 17074 | 0 | −1.426 |
| EIF2B2 | 4 | 0.00014712 | 0.00056023 | 0.026827 | 376 | 4 | −2.0731 | 0.99985 | 0.99986 | 1 | 17779 | 0 | −2.0731 |
| PSMD13 | 4 | 0.00014823 | 0.00056462 | 0.026966 | 377 | 4 | −1.5591 | 0.99971 | 0.99972 | 1 | 17701 | 0 | −1.5591 |
| EXOSC9 | 4 | 0.00015008 | 0.0005712 | 0.027208 | 378 | 4 | −1.3327 | 0.99985 | 0.99986 | 1 | 17777 | 0 | −1.3327 |
| NARS2 | 4 | 0.00015177 | 0.00058052 | 0.027579 | 379 | 4 | −1.2072 | 0.99764 | 0.99766 | 1 | 17342 | 0 | −1.2072 |
| BRAP | 4 | 0.00015316 | 0.00058656 | 0.027777 | 380 | 4 | −1.0996 | 0.99985 | 0.99986 | 1 | 17774 | 0 | −1.0996 |
| NDOR1 | 4 | 0.00015371 | 0.00058875 | 0.027777 | 381 | 4 | −1.8415 | 0.99985 | 0.99986 | 1 | 17773 | 0 | −1.8415 |
| WIPI2 | 4 | 0.00015378 | 0.0005893 | 0.027777 | 382 | 4 | −1.682 | 0.99985 | 0.99986 | 1 | 17772 | 0 | −1.682 |
| WDR1 | 4 | 0.00015683 | 0.00059862 | 0.027821 | 383 | 4 | −1.2676 | 0.99314 | 0.99325 | 1 | 17083 | 0 | −1.2676 |
| TBL3 | 4 | 0.0001582 | 0.00060136 | 0.027821 | 384 | 4 | −1.2294 | 0.99984 | 0.99985 | 1 | 17771 | 0 | −1.2294 |
| TNPO1 | 4 | 0.00015844 | 0.00060191 | 0.027821 | 385 | 4 | −1.1539 | 0.99984 | 0.99985 | 1 | 17770 | 0 | −1.1539 |
| ACTR1A | 4 | 0.00015864 | 0.00060191 | 0.027821 | 386 | 3 | −1.7053 | 0.62948 | 0.74234 | 1 | 12368 | 1 | −1.7053 |
| SELRC1 | 4 | 0.00015903 | 0.00060356 | 0.027821 | 387 | 3 | −1.2156 | 0.37454 | 0.54227 | 1 | 9389 | 1 | −1.2156 |
| PNKP | 4 | 0.00015931 | 0.00060411 | 0.027821 | 388 | 4 | −1.0961 | 0.99984 | 0.99985 | 1 | 17769 | 0 | −1.0961 |
| CYC1 | 4 | 0.00016058 | 0.00060959 | 0.027931 | 389 | 4 | −1.4952 | 0.99909 | 0.99907 | 1 | 17511 | 0 | −1.4952 |
| BCL2L1 | 4 | 0.00016078 | 0.00060959 | 0.027931 | 390 | 3 | −1.7936 | 0.93918 | 0.93906 | 1 | 15720 | 0 | −1.7936 |
| PRPF4 | 4 | 0.00016557 | 0.00062769 | 0.028688 | 391 | 4 | −1.4325 | 0.99983 | 0.99985 | 1 | 17768 | 0 | −1.4325 |
| PDSS1 | 4 | 0.00016643 | 0.00062988 | 0.028715 | 392 | 4 | −1.4558 | 0.99971 | 0.99972 | 1 | 17702 | 0 | −1.4558 |
| MRPS35 | 4 | 0.00016714 | 0.00063263 | 0.028748 | 393 | 4 | −0.88958 | 0.99983 | 0.99984 | 1 | 17767 | 0 | −0.88958 |
| PRPF40A | 4 | 0.00016811 | 0.00063537 | 0.028748 | 394 | 4 | −1.8833 | 0.99856 | 0.99857 | 1 | 17435 | 0 | −1.8833 |
| POLR3K | 4 | 0.00016822 | 0.00063537 | 0.028748 | 395 | 4 | −1.0408 | 0.99983 | 0.99984 | 1 | 17766 | 0 | −1.0408 |
| VPS45 | 4 | 0.00016956 | 0.00064305 | 0.028786 | 396 | 4 | −1.0884 | 0.99983 | 0.99984 | 1 | 17765 | 0 | −1.0884 |
| INTS6 | 4 | 0.00016973 | 0.00064359 | 0.028786 | 397 | 4 | −1.7784 | 0.99983 | 0.99984 | 1 | 17764 | 0 | −1.7784 |
| MCM2 | 4 | 0.00016993 | 0.00064359 | 0.028786 | 398 | 4 | −1.9134 | 0.99939 | 0.99941 | 1 | 17590 | 0 | −1.9134 |
| BTAF1 | 4 | 0.00016998 | 0.00064359 | 0.028786 | 399 | 4 | −0.92453 | 0.99983 | 0.99984 | 1 | 17763 | 0 | −0.92453 |
| FAM210A | 4 | 0.00017057 | 0.00064524 | 0.028786 | 400 | 4 | −1.1768 | 0.99983 | 0.99984 | 1 | 17762 | 0 | −1.1768 |
| NCAPG | 4 | 0.00017099 | 0.00064579 | 0.028786 | 401 | 4 | −1.2454 | 0.99983 | 0.99984 | 1 | 17761 | 0 | −1.2454 |
| AHCY | 4 | 0.00017155 | 0.00064908 | 0.028862 | 402 | 3 | −1.3227 | 0.89217 | 0.89191 | 1 | 14823 | 1 | −1.3227 |
| NAE1 | 4 | 0.00017339 | 0.00065456 | 0.029034 | 403 | 4 | −1.2834 | 0.99628 | 0.99636 | 1 | 17230 | 0 | −1.2834 |
| NDUFAF6 | 4 | 0.00017649 | 0.00066444 | 0.0294 | 404 | 3 | −1.022 | 0.96697 | 0.96688 | 1 | 16261 | 0 | −1.022 |
| MRPL4 | 4 | 0.00017784 | 0.00066937 | 0.029546 | 405 | 4 | −1.1739 | 0.99982 | 0.99983 | 1 | 17760 | 0 | −1.1739 |
| HMBS | 4 | 0.00017815 | 0.00067156 | 0.02957 | 406 | 3 | −1.4684 | 0.80772 | 0.82765 | 1 | 13712 | 1 | −1.4684 |
| MRPS24 | 4 | 0.00017871 | 0.00067321 | 0.02957 | 407 | 4 | −1.2883 | 0.99982 | 0.99983 | 1 | 17759 | 0 | −1.2883 |
| KIAA0020 | 4 | 0.00018011 | 0.0006765 | 0.029643 | 408 | 4 | −1.574 | 0.99982 | 0.99983 | 1 | 17758 | 0 | −1.574 |
| NUP62 | 4 | 0.0001817 | 0.00068418 | 0.029907 | 409 | 4 | −0.89472 | 0.99982 | 0.99983 | 1 | 17757 | 0 | −0.89472 |
| UBIAD1 | 4 | 0.0001845 | 0.00069679 | 0.030213 | 410 | 4 | −1.0987 | 0.99835 | 0.99837 | 1 | 17415 | 0 | −1.0987 |
| FDXR | 4 | 0.00018463 | 0.00069734 | 0.030213 | 411 | 4 | −1.2415 | 0.99982 | 0.99982 | 1 | 17756 | 0 | −1.2415 |
| RPUSD4 | 4 | 0.00018471 | 0.00069734 | 0.030213 | 412 | 4 | −1.4852 | 0.98735 | 0.98744 | 1 | 16824 | 0 | −1.4852 |
| MFAP1 | 4 | 0.00018499 | 0.00069789 | 0.030213 | 413 | 4 | −1.2223 | 0.99982 | 0.99982 | 1 | 17755 | 0 | −1.2223 |
| SF3B4 | 4 | 0.00018796 | 0.00070831 | 0.030542 | 414 | 4 | −1.3204 | 0.99981 | 0.99982 | 1 | 17754 | 0 | −1.3204 |
| FTSJ2 | 4 | 0.00018842 | 0.00070886 | 0.030542 | 415 | 4 | −1.5306 | 0.99981 | 0.99982 | 1 | 17753 | 0 | −1.5306 |
| EARS2 | 4 | 0.00019011 | 0.00071379 | 0.030681 | 416 | 3 | −0.98441 | 0.88022 | 0.88077 | 1 | 14636 | 1 | −0.98441 |
| SBDS | 4 | 0.00019077 | 0.00071709 | 0.03075 | 417 | 4 | −1.6938 | 0.99916 | 0.99915 | 1 | 17530 | 0 | −1.6938 |
| CENPN | 4 | 0.00019142 | 0.00071928 | 0.03077 | 418 | 4 | −1.6007 | 0.99817 | 0.99821 | 1 | 17394 | 0 | −1.6007 |
| RAD1 | 4 | 0.0001923 | 0.00072202 | 0.03081 | 419 | 4 | −1.341 | 0.99528 | 0.99537 | 1 | 17178 | 0 | −1.341 |
| MRPL21 | 4 | 0.00019327 | 0.00072422 | 0.03081 | 420 | 4 | −0.96031 | 0.99981 | 0.99982 | 1 | 17752 | 0 | −0.96031 |
| SUGT1 | 4 | 0.00019362 | 0.00072531 | 0.03081 | 421 | 4 | −2.1672 | 0.99874 | 0.99873 | 1 | 17458 | 0 | −2.1672 |
| GMPPB | 3 | 0.00019452 | 0.00059094 | 0.027782 | 422 | 3 | −1.3293 | 0.99981 | 0.9998 | 1 | 17751 | 0 | −1.3293 |
| GNB1L | 3 | 0.0001958 | 0.00059588 | 0.027821 | 423 | 3 | −1.2515 | 0.9998 | 0.9998 | 1 | 17750 | 0 | −1.2515 |
| MIS18A | 3 | 0.00019724 | 0.00059698 | 0.027821 | 424 | 3 | −1.4495 | 0.9998 | 0.9998 | 1 | 17749 | 0 | −1.4495 |
| CCT2 | 4 | 0.00020031 | 0.00075219 | 0.031876 | 425 | 4 | −2.261 | 0.9998 | 0.99981 | 1 | 17748 | 0 | −2.261 |
| SSRP1 | 4 | 0.00020145 | 0.00075548 | 0.031907 | 426 | 4 | −1.841 | 0.99947 | 0.99949 | 1 | 17610 | 0 | −1.841 |
| LIAS | 4 | 0.00020168 | 0.00075657 | 0.031907 | 427 | 4 | −1.7482 | 0.99966 | 0.99968 | 1 | 17687 | 0 | −1.7482 |
| CMTR1 | 4 | 0.0002019 | 0.00075822 | 0.031907 | 428 | 3 | −1.1255 | 0.87111 | 0.87303 | 1 | 14485 | 1 | −1.1255 |
| ATP2A2 | 4 | 0.00020607 | 0.00077138 | 0.032281 | 429 | 4 | −1.6915 | 0.99979 | 0.99981 | 1 | 17747 | 0 | −1.6915 |
| SKA1 | 4 | 0.00020636 | 0.00077193 | 0.032281 | 430 | 4 | −2.9167 | 0.99979 | 0.99981 | 1 | 17746 | 0 | −2.9167 |
| PPP4C | 4 | 0.00020717 | 0.00077248 | 0.032281 | 431 | 4 | −1.801 | 0.99939 | 0.9994 | 1 | 17587 | 0 | −1.801 |
| C19orf53 | 4 | 0.00020902 | 0.00077851 | 0.032458 | 432 | 4 | −1.7462 | 0.99686 | 0.9969 | 1 | 17276 | 0 | −1.7462 |
| ATP6AP2 | 4 | 0.00021018 | 0.000784 | 0.03253 | 433 | 4 | −1.4633 | 0.99919 | 0.99918 | 1 | 17541 | 0 | −1.4633 |
| CNOT1 | 4 | 0.00021087 | 0.00078564 | 0.03253 | 434 | 4 | −1.1357 | 0.99979 | 0.9998 | 1 | 17745 | 0 | −1.1357 |
| C19orf52 | 4 | 0.00021088 | 0.00078564 | 0.03253 | 435 | 4 | −1.1122 | 0.99811 | 0.99814 | 1 | 17388 | 0 | −1.1122 |
| WDR46 | 4 | 0.00021276 | 0.00078948 | 0.032614 | 436 | 4 | −1.0579 | 0.99979 | 0.9998 | 1 | 17744 | 0 | −1.0579 |
| AP1G1 | 4 | 0.00021417 | 0.00079496 | 0.032766 | 437 | 3 | −1.6443 | 0.94935 | 0.94926 | 1 | 15910 | 0 | −1.6443 |
| CCT5 | 4 | 0.00021606 | 0.0007999 | 0.032894 | 438 | 4 | −1.25 | 0.99978 | 0.99979 | 1 | 17741 | 0 | −1.25 |
| NDUFAF4 | 4 | 0.00021797 | 0.00080758 | 0.033104 | 439 | 4 | −1.5305 | 0.9993 | 0.99931 | 1 | 17562 | 0 | −1.5305 |
| NAF1 | 4 | 0.0002182 | 0.00080868 | 0.033104 | 440 | 4 | −1.317 | 0.99838 | 0.9984 | 1 | 17420 | 0 | −1.317 |
| GRPEL1 | 4 | 0.00022036 | 0.00081635 | 0.033215 | 441 | 4 | −1.5899 | 0.99273 | 0.99284 | 1 | 17066 | 0 | −1.5899 |
| PMPCB | 4 | 0.0002206 | 0.00081635 | 0.033215 | 442 | 4 | −1.4572 | 0.99974 | 0.99975 | 1 | 17719 | 0 | −1.4572 |
| EIF2AK4 | 4 | 0.00022082 | 0.0008169 | 0.033215 | 443 | 4 | −1.1053 | 0.99978 | 0.99978 | 1 | 17740 | 0 | −1.1053 |
| RFC3 | 4 | 0.00022329 | 0.00082732 | 0.033563 | 444 | 4 | −2.1244 | 0.99978 | 0.99978 | 1 | 17739 | 0 | −2.1244 |
| ZNF259 | 4 | 0.0002252 | 0.0008339 | 0.033754 | 445 | 3 | −1.4653 | 0.91433 | 0.9141 | 1 | 15230 | 0 | −1.4653 |
| NSUN4 | 4 | 0.00022569 | 0.00083692 | 0.033801 | 446 | 4 | −1.0102 | 0.99962 | 0.99963 | 1 | 17661 | 0 | −1.0102 |
| PUF60 | 4 | 0.00022643 | 0.00084103 | 0.033891 | 447 | 4 | −1.3181 | 0.99935 | 0.99937 | 1 | 17577 | 0 | −1.3181 |
| ACTR3 | 4 | 0.00022796 | 0.00084761 | 0.03408 | 448 | 4 | −0.80572 | 0.99977 | 0.99978 | 1 | 17737 | 0 | −0.80572 |
| C9orf114 | 4 | 0.00022839 | 0.00085036 | 0.034114 | 449 | 4 | −2.1346 | 0.9993 | 0.99932 | 1 | 17563 | 0 | −2.1346 |
| USP7 | 4 | 0.00022987 | 0.00085694 | 0.034248 | 450 | 4 | −1.0894 | 0.9996 | 0.99962 | 1 | 17653 | 0 | −1.0894 |
| SPC25 | 4 | 0.00023017 | 0.00085749 | 0.034248 | 451 | 4 | −1.2857 | 0.99977 | 0.99977 | 1 | 17736 | 0 | −1.2857 |
| DLD | 4 | 0.0002316 | 0.00086297 | 0.034297 | 452 | 4 | −1.1326 | 0.99867 | 0.99868 | 1 | 17447 | 0 | −1.1326 |
| TRMT5 | 4 | 0.00023311 | 0.00086681 | 0.034297 | 453 | 2 | −1.3994 | 0.93716 | 0.937 | 1 | 15662 | 0 | −1.3994 |
| ZNF407 | 4 | 0.00023335 | 0.00086736 | 0.034297 | 454 | 3 | −1.6507 | 0.91038 | 0.91014 | 1 | 15165 | 0 | −1.6507 |
| PARN | 4 | 0.00023335 | 0.00086736 | 0.034297 | 455 | 4 | −0.86018 | 0.99977 | 0.99977 | 1 | 17735 | 0 | −0.86018 |
| WDR55 | 4 | 0.00023378 | 0.00086845 | 0.034297 | 456 | 4 | −0.76337 | 0.99977 | 0.99977 | 1 | 17734 | 0 | −0.76337 |
| AASDHPPT | 4 | 0.00023415 | 0.0008701 | 0.034297 | 457 | 3 | −0.94667 | 0.69828 | 0.77045 | 1 | 12803 | 1 | −0.94667 |
| UMPS | 4 | 0.00023535 | 0.00087504 | 0.034384 | 458 | 4 | −1.6164 | 0.98369 | 0.98375 | 1 | 16679 | 0 | −1.6164 |
| TSR2 | 4 | 0.00023624 | 0.00087613 | 0.034384 | 459 | 4 | −1.7223 | 0.99976 | 0.99977 | 1 | 17733 | 0 | −1.7223 |
| NXT1 | 4 | 0.00023775 | 0.00088052 | 0.034482 | 460 | 4 | −0.93699 | 0.99976 | 0.99977 | 1 | 17731 | 0 | −0.93699 |
| PCBP2 | 4 | 0.00023888 | 0.00088546 | 0.0346 | 461 | 3 | −1.6693 | 0.90007 | 0.89971 | 1 | 14967 | 0 | −1.6693 |
| PSMB2 | 4 | 0.00023992 | 0.0008882 | 0.034632 | 462 | 4 | −1.9007 | 0.99976 | 0.99976 | 1 | 17730 | 0 | −1.9007 |
| EIF5A | 2 | 0.00024167 | 0.00046809 | 0.024566 | 463 | 2 | −1.6868 | 0.99976 | 0.99975 | 1 | 17729 | 0 | −1.6868 |
| PDRG1 | 4 | 0.00024178 | 0.00089204 | 0.034707 | 464 | 4 | −1.3767 | 0.99976 | 0.99976 | 1 | 17727 | 0 | −1.3767 |
| STIL | 4 | 0.00024486 | 0.00090026 | 0.03495 | 465 | 4 | −1.1226 | 0.99976 | 0.99976 | 1 | 17726 | 0 | −1.1226 |
| POT1 | 4 | 0.00024584 | 0.00090356 | 0.03495 | 466 | 4 | −1.2743 | 0.99947 | 0.99949 | 1 | 17613 | 0 | −1.2743 |
| ARMC5 | 4 | 0.00024605 | 0.0009041 | 0.03495 | 467 | 4 | −1.3498 | 0.98994 | 0.99003 | 1 | 16925 | 0 | −1.3498 |
| SCAP | 4 | 0.00024652 | 0.0009063 | 0.03496 | 468 | 4 | −1.1205 | 0.99975 | 0.99976 | 1 | 17723 | 0 | −1.1205 |
| C15orf57 | 4 | 0.0002492 | 0.00091233 | 0.035118 | 469 | 4 | −0.80999 | 0.99975 | 0.99975 | 1 | 17722 | 0 | −0.80999 |
| FANCF | 4 | 0.00025257 | 0.00092659 | 0.035515 | 470 | 4 | −0.85695 | 0.99975 | 0.99975 | 1 | 17721 | 0 | −0.85695 |
| MED8 | 4 | 0.00025269 | 0.00092659 | 0.035515 | 471 | 4 | −0.92398 | 0.99975 | 0.99975 | 1 | 17720 | 0 | −0.92398 |
| MRPL23 | 4 | 0.00025401 | 0.00093098 | 0.035608 | 472 | 2 | −0.17767 | 0.5793 | 0.72128 | 1 | 12046 | 2 | −0.17767 |
| DSCC1 | 4 | 0.00025654 | 0.00093975 | 0.035868 | 473 | 4 | −1.0888 | 0.97924 | 0.97933 | 1 | 16560 | 0 | −1.0888 |
| ATP7A | 4 | 0.00025977 | 0.00095237 | 0.036272 | 474 | 4 | −1.0755 | 0.99974 | 0.99975 | 1 | 17718 | 0 | −1.0755 |
| TPX2 | 4 | 0.00026024 | 0.00095456 | 0.036279 | 475 | 4 | −1.5798 | 0.99974 | 0.99975 | 1 | 17717 | 0 | −1.5798 |
| MED20 | 4 | 0.00026198 | 0.00096224 | 0.036439 | 476 | 4 | −0.79453 | 0.99974 | 0.99974 | 1 | 17716 | 0 | −0.79453 |
| TCEB2 | 4 | 0.0002627 | 0.00096279 | 0.036439 | 477 | 3 | −1.8123 | 0.9639 | 0.96381 | 1 | 16194 | 0 | −1.8123 |
| KANSL1 | 4 | 0.00026396 | 0.00097101 | 0.036673 | 478 | 4 | −1.0471 | 0.99974 | 0.99974 | 1 | 17715 | 0 | −1.0471 |
| PSMB6 | 4 | 0.00026619 | 0.00097814 | 0.03685 | 479 | 4 | −0.92789 | 0.99973 | 0.99974 | 1 | 17714 | 0 | −0.92789 |
| NSF | 4 | 0.00026666 | 0.00097979 | 0.03685 | 480 | 4 | −1.9123 | 0.99973 | 0.99974 | 1 | 17713 | 0 | −1.9123 |
| CHMP7 | 4 | 0.0002678 | 0.00098198 | 0.036856 | 481 | 4 | −1.1318 | 0.99861 | 0.99863 | 1 | 17442 | 0 | −1.1318 |
| STRAP | 4 | 0.00026923 | 0.00098582 | 0.036923 | 482 | 4 | −1.2446 | 0.99934 | 0.99935 | 1 | 17571 | 0 | −1.2446 |
| DHX9 | 4 | 0.00027199 | 0.0010001 | 0.03738 | 483 | 4 | −1.7348 | 0.99909 | 0.99908 | 1 | 17513 | 0 | −1.7348 |
| CENPO | 4 | 0.00027283 | 0.0010023 | 0.037384 | 484 | 4 | −1.1389 | 0.99973 | 0.99973 | 1 | 17710 | 0 | −1.1389 |
| OTUD5 | 4 | 0.00027448 | 0.0010072 | 0.037491 | 485 | 4 | −1.282 | 0.99934 | 0.99935 | 1 | 17570 | 0 | −1.282 |
| KPTN | 4 | 0.00027596 | 0.0010116 | 0.037577 | 486 | 4 | −0.59868 | 0.99972 | 0.99973 | 1 | 17709 | 0 | −0.59868 |
| VARS | 4 | 0.00027693 | 0.0010138 | 0.037581 | 487 | 4 | −2.3787 | 0.99972 | 0.99973 | 1 | 17708 | 0 | −2.3787 |
| CNOT11 | 4 | 0.00028177 | 0.0010308 | 0.038075 | 488 | 4 | −1.3357 | 0.97343 | 0.97338 | 1 | 16415 | 0 | −1.3357 |
| EAF1 | 4 | 0.00028205 | 0.0010313 | 0.038075 | 489 | 4 | −1.8157 | 0.99021 | 0.99029 | 1 | 16937 | 0 | −1.8157 |
| LONP1 | 4 | 0.00028304 | 0.0010379 | 0.03824 | 490 | 4 | −1.436 | 0.99972 | 0.99973 | 1 | 17707 | 0 | −1.436 |
| NAPG | 4 | 0.00028576 | 0.0010478 | 0.038507 | 491 | 4 | −1.4247 | 0.99971 | 0.99972 | 1 | 17706 | 0 | −1.4247 |
| PPP6C | 4 | 0.00028613 | 0.0010494 | 0.038507 | 492 | 4 | −1.5183 | 0.99971 | 0.99972 | 1 | 17705 | 0 | −1.5183 |
| HNRNPU | 4 | 0.00028918 | 0.0010588 | 0.038654 | 493 | 4 | −2.1069 | 0.9837 | 0.98376 | 1 | 16680 | 0 | −2.1069 |
| ATG9A | 4 | 0.00028925 | 0.0010593 | 0.038654 | 494 | 4 | −0.83008 | 0.99971 | 0.99972 | 1 | 17704 | 0 | −0.83008 |
| AMBRA1 | 4 | 0.00028988 | 0.0010599 | 0.038654 | 495 | 4 | −0.84124 | 0.99971 | 0.99972 | 1 | 17703 | 0 | −0.84124 |
| ELP5 | 4 | 0.00029091 | 0.0010642 | 0.038736 | 496 | 4 | −1.3119 | 0.99901 | 0.99898 | 1 | 17494 | 0 | −1.3119 |
| SLC25A19 | 4 | 0.00029585 | 0.0010829 | 0.039256 | 497 | 3 | −1.3623 | 0.91113 | 0.91093 | 1 | 15182 | 0 | −1.3623 |
| POLE2 | 4 | 0.00029614 | 0.0010829 | 0.039256 | 498 | 3 | −1.8393 | 0.82385 | 0.83789 | 1 | 13911 | 1 | −1.8393 |
| COX15 | 4 | 0.00029812 | 0.0010895 | 0.039397 | 499 | 4 | −0.96016 | 0.9997 | 0.99971 | 1 | 17700 | 0 | −0.96016 |
| SKIV2L2 | 4 | 0.00029978 | 0.0010939 | 0.039397 | 500 | 4 | −1.0626 | 0.9997 | 0.99971 | 1 | 17699 | 0 | −1.0626 |
| MRPL35 | 4 | 0.00030011 | 0.0010944 | 0.039397 | 501 | 4 | −1.1441 | 0.9996 | 0.99961 | 1 | 17647 | 0 | −1.1441 |
| GINS3 | 4 | 0.00030054 | 0.0010955 | 0.039397 | 502 | 4 | −1.2775 | 0.99793 | 0.99797 | 1 | 17369 | 0 | −1.2775 |
| URB2 | 4 | 0.00030198 | 0.0011021 | 0.039516 | 503 | 4 | −1.1709 | 0.9997 | 0.99971 | 1 | 17698 | 0 | −1.1709 |
| PSMB5 | 4 | 0.00030249 | 0.0011032 | 0.039516 | 504 | 4 | −1.0432 | 0.9997 | 0.99971 | 1 | 17697 | 0 | −1.0432 |
| PTDSS1 | 4 | 0.00030327 | 0.001105 | 0.039516 | 505 | 4 | −1.1186 | 0.9997 | 0.99971 | 1 | 17696 | 0 | −1.1186 |
| USP 5 | 4 | 0.00030444 | 0.0011092 | 0.039575 | 506 | 4 | −0.90047 | 0.9997 | 0.99971 | 1 | 17695 | 0 | −0.90047 |
| MED30 | 4 | 0.00030718 | 0.0011158 | 0.039731 | 507 | 4 | −2.9162 | 0.99969 | 0.99971 | 1 | 17694 | 0 | −2.9162 |
| CSDE1 | 4 | 0.00030915 | 0.0011213 | 0.039848 | 508 | 4 | −0.66912 | 0.99969 | 0.99971 | 1 | 17693 | 0 | −0.66912 |
| MRPL20 | 4 | 0.00031045 | 0.0011273 | 0.039983 | 509 | 2 | −1.1652 | 0.45269 | 0.61003 | 1 | 10372 | 1 | −1.1652 |
| PCYT2 | 4 | 0.00031285 | 0.0011355 | 0.040196 | 510 | 4 | −1.4009 | 0.99969 | 0.9997 | 1 | 17692 | 0 | −1.4009 |
| EIF1AX | 4 | 0.00031737 | 0.001152 | 0.040699 | 511 | 3 | −1.6119 | 0.073287 | 0.17609 | 0.872163 | 3598 | 1 | −1.6119 |
| CXorf56 | 4 | 0.00031833 | 0.0011567 | 0.040784 | 512 | 4 | −0.87861 | 0.99968 | 0.9997 | 1 | 17691 | 0 | −0.87861 |
| VPS52 | 4 | 0.00032074 | 0.0011641 | 0.040965 | 513 | 4 | −1.149 | 0.99861 | 0.99862 | 1 | 17441 | 0 | −1.149 |
| CDK1 | 4 | 0.00032383 | 0.0011712 | 0.041135 | 514 | 4 | −2.4607 | 0.99966 | 0.99968 | 1 | 17684 | 0 | −2.4607 |
| ZNHIT3 | 4 | 0.00032507 | 0.0011767 | 0.041248 | 515 | 4 | −1.1597 | 0.99226 | 0.99238 | 1 | 17042 | 0 | −1.1597 |
| LRPPRC | 4 | 0.00032881 | 0.0011893 | 0.041609 | 516 | 3 | −1.0368 | 0.97058 | 0.97052 | 1 | 16342 | 0 | −1.0368 |
| CSTF3 | 4 | 0.00033048 | 0.0011931 | 0.041663 | 517 | 4 | −1.5587 | 0.99967 | 0.99969 | 1 | 17690 | 0 | −1.5587 |
| ENY2 | 4 | 0.00033298 | 0.0012014 | 0.041869 | 518 | 3 | −1.1352 | 0.038469 | 0.11282 | 0.792322 | 2529 | 1 | −1.1352 |
| C21orf59 | 4 | 0.00033493 | 0.0012123 | 0.042089 | 519 | 4 | −1.6226 | 0.99967 | 0.99968 | 1 | 17689 | 0 | −1.6226 |
| EIF3A | 4 | 0.00033563 | 0.0012145 | 0.042089 | 520 | 4 | −1.3899 | 0.99966 | 0.99968 | 1 | 17688 | 0 | −1.3899 |
| TAF1C | 4 | 0.00033605 | 0.0012151 | 0.042089 | 521 | 4 | −1.2849 | 0.99966 | 0.99968 | 1 | 17686 | 0 | −1.2849 |
| TKT | 4 | 0.00033702 | 0.0012178 | 0.042089 | 522 | 4 | −1.1184 | 0.99315 | 0.99326 | 1 | 17084 | 0 | −1.1184 |
| TRMT61A | 4 | 0.000338 | 0.00122 | 0.042089 | 523 | 4 | −0.93002 | 0.97981 | 0.9799 | 1 | 16580 | 0 | −0.93002 |
| CHCHD4 | 4 | 0.00033844 | 0.0012217 | 0.042089 | 524 | 4 | −1.1594 | 0.99966 | 0.99968 | 1 | 17685 | 0 | −1.1594 |
| RCC1 | 4 | 0.00034021 | 0.0012288 | 0.042187 | 525 | 4 | −1.1492 | 0.99954 | 0.99956 | 1 | 17626 | 0 | −1.1492 |
| POLRMT | 4 | 0.00034053 | 0.0012299 | 0.042187 | 526 | 4 | −1.1347 | 0.99941 | 0.99943 | 1 | 17593 | 0 | −1.1347 |
| RPS11 | 4 | 0.00034169 | 0.0012315 | 0.042187 | 527 | 4 | −2.0046 | 0.99966 | 0.99968 | 1 | 17683 | 0 | −2.0046 |
| NUDC | 4 | 0.00034582 | 0.001248 | 0.04267 | 528 | 4 | −1.3826 | 0.99965 | 0.99967 | 1 | 17682 | 0 | −1.3826 |
| NOP2 | 4 | 0.00035154 | 0.0012666 | 0.043226 | 529 | 4 | −1.3947 | 0.99947 | 0.99948 | 1 | 17609 | 0 | −1.3947 |
| MCRS1 | 4 | 0.00035435 | 0.001277 | 0.043499 | 530 | 4 | −1.1564 | 0.99965 | 0.99966 | 1 | 17681 | 0 | −1.1564 |
| ARPC4 | 4 | 0.00035522 | 0.0012823 | 0.043594 | 531 | 4 | −0.71059 | 0.99964 | 0.99966 | 1 | 17680 | 0 | −0.71059 |
| RTN4IP1 | 4 | 0.0003568 | 0.0012913 | 0.043774 | 532 | 3 | −1.2163 | 0.092177 | 0.20879 | 0.899809 | 4141 | 1 | −1.2163 |
| RPS21 | 4 | 0.00035727 | 0.0012924 | 0.043774 | 533 | 4 | −1.1102 | 0.99964 | 0.99966 | 1 | 17678 | 0 | −1.1102 |
| CD151 | 4 | 0.00035992 | 0.0012973 | 0.043825 | 534 | 4 | −1.3582 | 0.99964 | 0.99965 | 1 | 17676 | 0 | −1.3582 |
| SMC2 | 4 | 0.00036036 | 0.0012995 | 0.043825 | 535 | 4 | −0.8273 | 0.99964 | 0.99965 | 1 | 17675 | 0 | −0.8273 |
| TACC3 | 4 | 0.00036066 | 0.0013012 | 0.043825 | 536 | 4 | −1.0301 | 0.99964 | 0.99965 | 1 | 17674 | 0 | −1.0301 |
| N6AMT1 | 4 | 0.00036311 | 0.0013149 | 0.044087 | 537 | 4 | −1.4465 | 0.99848 | 0.9985 | 1 | 17430 | 0 | −1.4465 |
| EXOC7 | 4 | 0.00036362 | 0.0013176 | 0.044087 | 538 | 4 | −1.006 | 0.99964 | 0.99965 | 1 | 17673 | 0 | −1.006 |
| TARS2 | 4 | 0.00036378 | 0.0013187 | 0.044087 | 539 | 4 | −1.6008 | 0.99718 | 0.99721 | 1 | 17300 | 0 | −1.6008 |
| GGPS1 | 4 | 0.00036713 | 0.0013374 | 0.044482 | 540 | 4 | −1.9521 | 0.99927 | 0.99927 | 1 | 17555 | 0 | −1.9521 |
| EEF1D | 4 | 0.00036916 | 0.0013445 | 0.044482 | 541 | 3 | −0.91363 | 0.94595 | 0.94583 | 1 | 15847 | 0 | −0.91363 |
| EFTUD1 | 4 | 0.0003699 | 0.0013461 | 0.044482 | 542 | 4 | −1.7741 | 0.99963 | 0.99964 | 1 | 17672 | 0 | −1.7741 |
| RPL14 | 4 | 0.00037005 | 0.0013461 | 0.044482 | 543 | 4 | −1.5901 | 0.99963 | 0.99964 | 1 | 17671 | 0 | −1.5901 |
| DNAJC9 | 4 | 0.00037036 | 0.0013467 | 0.044482 | 544 | 4 | −0.86029 | 0.99963 | 0.99964 | 1 | 17670 | 0 | −0.86029 |
| ACAD9 | 4 | 0.00037051 | 0.0013478 | 0.044482 | 545 | 4 | −0.76256 | 0.99963 | 0.99964 | 1 | 17669 | 0 | −0.76256 |
| HUS1 | 4 | 0.00037444 | 0.0013577 | 0.044726 | 546 | 4 | −0.97101 | 0.99963 | 0.99964 | 1 | 17668 | 0 | −0.97101 |
| SYS1 | 4 | 0.00037664 | 0.0013648 | 0.044879 | 547 | 4 | −1.1233 | 0.99961 | 0.99962 | 1 | 17659 | 0 | −1.1233 |
| CLTC | 4 | 0.00037795 | 0.0013703 | 0.044914 | 548 | 4 | −0.88658 | 0.99962 | 0.99963 | 1 | 17667 | 0 | −0.88658 |
| MICALL2 | 4 | 0.00037826 | 0.0013708 | 0.044914 | 549 | 4 | −0.90957 | 0.99962 | 0.99963 | 1 | 17666 | 0 | −0.90957 |
| THOC1 | 4 | 0.00037973 | 0.0013785 | 0.04492 | 550 | 4 | −1.7177 | 0.9966 | 0.99666 | 1 | 17254 | 0 | −1.7177 |
| EIF4H | 4 | 0.0003804 | 0.0013807 | 0.04492 | 551 | 4 | −0.95844 | 0.99962 | 0.99963 | 1 | 17664 | 0 | −0.95844 |
| VRK1 | 4 | 0.00038079 | 0.0013812 | 0.04492 | 552 | 4 | −1.1652 | 0.98983 | 0.98991 | 1 | 16919 | 0 | −1.1652 |
| CDK5 | 4 | 0.00038117 | 0.0013834 | 0.04492 | 553 | 4 | −0.81642 | 0.99962 | 0.99963 | 1 | 17663 | 0 | −0.81642 |
| SETD1A | 4 | 0.00038133 | 0.0013834 | 0.04492 | 554 | 4 | −2.0939 | 0.99962 | 0.99963 | 1 | 17662 | 0 | −2.0939 |
| VMP1 | 4 | 0.00038353 | 0.0013873 | 0.044963 | 555 | 4 | −1.199 | 0.97272 | 0.97269 | 1 | 16400 | 0 | −1.199 |
| POLR2E | 4 | 0.00038566 | 0.0013944 | 0.045104 | 556 | 4 | −1.2625 | 0.99961 | 0.99963 | 1 | 17660 | 0 | −1.2625 |
| NARFL | 4 | 0.0003863 | 0.0013966 | 0.045104 | 557 | 3 | −2.0697 | 0.9548 | 0.95462 | 1 | 16008 | 0 | −2.0697 |
| IPO11 | 4 | 0.0003894 | 0.001407 | 0.045248 | 558 | 4 | −1.1934 | 0.99961 | 0.99962 | 1 | 17658 | 0 | −1.1934 |
| MRPL51 | 4 | 0.00038944 | 0.0014081 | 0.045248 | 559 | 4 | −1.3977 | 0.99943 | 0.99945 | 1 | 17598 | 0 | −1.3977 |
| DRD5 | 4 | 0.00039003 | 0.0014114 | 0.045248 | 560 | 4 | −1.2743 | 0.99961 | 0.99962 | 1 | 17657 | 0 | −1.2743 |
| IER3IP1 | 4 | 0.0003905 | 0.0014131 | 0.045248 | 561 | 4 | −0.77111 | 0.99961 | 0.99962 | 1 | 17656 | 0 | −0.77111 |
| ATP5H | 4 | 0.00039081 | 0.0014136 | 0.045248 | 562 | 4 | −0.92986 | 0.99961 | 0.99962 | 1 | 17655 | 0 | −0.92986 |
| LAGE3 | 4 | 0.00039301 | 0.0014218 | 0.045431 | 563 | 4 | −1.3268 | 0.99961 | 0.99962 | 1 | 17654 | 0 | −1.3268 |
| MCMBP | 4 | 0.00039425 | 0.0014262 | 0.04549 | 564 | 3 | −1.2935 | 0.93897 | 0.93885 | 1 | 15714 | 0 | −1.2935 |
| EIF3I | 4 | 0.00039897 | 0.0014394 | 0.045667 | 565 | 4 | −1.3281 | 0.99612 | 0.99621 | 1 | 17219 | 0 | −1.3281 |
| METTL16 | 4 | 0.00039919 | 0.001441 | 0.045667 | 566 | 4 | −1.2698 | 0.9996 | 0.99962 | 1 | 17652 | 0 | −1.2698 |
| MASTL | 4 | 0.00040004 | 0.0014449 | 0.045667 | 567 | 3 | −1.4615 | 0.90513 | 0.90479 | 1 | 15070 | 0 | −1.4615 |
| DDX51 | 4 | 0.00040039 | 0.001446 | 0.045667 | 568 | 4 | −1.5076 | 0.99038 | 0.99047 | 1 | 16945 | 0 | −1.5076 |
| MRPS9 | 4 | 0.00040175 | 0.0014487 | 0.045667 | 569 | 4 | −1.0734 | 0.9996 | 0.99962 | 1 | 17650 | 0 | −1.0734 |
| NARG2 | 4 | 0.00040207 | 0.0014503 | 0.045667 | 570 | 4 | −1.3633 | 0.9996 | 0.99962 | 1 | 17649 | 0 | −1.3633 |
| CCDC94 | 4 | 0.00040239 | 0.001452 | 0.045667 | 571 | 4 | −0.97832 | 0.9996 | 0.99962 | 1 | 17648 | 0 | −0.97832 |
| HINFP | 4 | 0.00040577 | 0.0014674 | 0.04607 | 572 | 4 | −0.8576 | 0.99959 | 0.99961 | 1 | 17646 | 0 | −0.8576 |
| MAU2 | 4 | 0.00041154 | 0.0014876 | 0.046545 | 573 | 3 | −1.0301 | 0.95231 | 0.95217 | 1 | 15964 | 0 | −1.0301 |
| MRPL34 | 4 | 0.00041299 | 0.0014937 | 0.046653 | 574 | 4 | −1.458 | 0.98897 | 0.98911 | 1 | 16880 | 0 | −1.458 |
| SUZ12 | 4 | 0.00041785 | 0.0015071 | 0.046991 | 575 | 4 | −0.93571 | 0.99958 | 0.9996 | 1 | 17645 | 0 | −0.93571 |
| SRPRB | 4 | 0.00042215 | 0.0015233 | 0.047414 | 576 | 4 | −0.91292 | 0.99325 | 0.99337 | 1 | 17090 | 0 | −0.91292 |
| VPS53 | 4 | 0.00042649 | 0.0015326 | 0.04547 | 577 | 4 | −0.69986 | 0.99957 | 0.99959 | 1 | 17644 | 0 | −0.69986 |
| RPL35 | 4 | 0.000427 | 0.0015354 | 0.047547 | 578 | 4 | −0.8092 | 0.99957 | 0.99959 | 1 | 17643 | 0 | −0.8092 |
| TXNL4A | 4 | 0.00042733 | 0.0015365 | 0.047547 | 579 | 4 | −1.8929 | 0.99957 | 0.99959 | 1 | 17642 | 0 | −1.8929 |
| ATRX | 4 | 0.00042771 | 0.0015381 | 0.047547 | 580 | 4 | −1.7393 | 0.98743 | 0.98752 | 1 | 16827 | 0 | −1.7393 |
| ATP6V0B | 4 | 0.00043219 | 0.0015513 | 0.047824 | 581 | 4 | −1.1768 | 0.9989 | 0.99888 | 1 | 17476 | 0 | −1.1768 |
| AATF | 4 | 0.00043273 | 0.0015524 | 0.047824 | 582 | 4 | −0.91994 | 0.99957 | 0.99959 | 1 | 17641 | 0 | −0.91994 |
| ADAT3 | 4 | 0.00043369 | 0.0015562 | 0.04786 | 583 | 4 | −1.2793 | 0.99876 | 0.99875 | 1 | 17461 | 0 | −1.2793 |
| PDHA1 | 4 | 0.00043578 | 0.0015617 | 0.047947 | 584 | 4 | −0.74985 | 0.99956 | 0.99958 | 1 | 17640 | 0 | −0.74985 |
| CHMP6 | 3 | 0.00043591 | 0.0013094 | 0.04402 | 585 | 3 | −1.2891 | 0.99956 | 0.99955 | 1 | 17639 | 0 | −1.2891 |
| RNF20 | 4 | 0.00043834 | 0.0015716 | 0.048169 | 586 | 4 | −0.9142 | 0.99956 | 0.99958 | 1 | 17637 | 0 | −0.9142 |
| RPS8 | 3 | 0.00044276 | 0.001328 | 0.044317 | 587 | 3 | −2.8705 | 0.99956 | 0.99955 | 1 | 17636 | 0 | −2.8705 |
| UQCRC2 | 4 | 0.00044332 | 0.0015918 | 0.048692 | 588 | 4 | −1.0409 | 0.99956 | 0.99958 | 1 | 17635 | 0 | −1.0409 |
| KRR1 | 4 | 0.00044391 | 0.001594 | 0.048692 | 589 | 3 | −1.4982 | 0.95455 | 0.95439 | 1 | 16005 | 0 | −1.4982 |
| QARS | 4 | 0.00044505 | 0.0015995 | 0.048777 | 590 | 4 | −1.1752 | 0.99955 | 0.99958 | 1 | 17634 | 0 | −1.1752 |
| RRM1 | 4 | 0.00045166 | 0.0016308 | 0.049613 | 591 | 4 | −1.5038 | 0.99955 | 0.99957 | 1 | 17633 | 0 | −1.5038 |
| ZNRD1 | 4 | 0.00045274 | 0.0016324 | 0.049613 | 592 | 4 | −1.5763 | 0.99908 | 0.99907 | 1 | 17509 | 0 | −1.5763 |
| VPS18 | 4 | 0.00045468 | 0.0016385 | 0.049713 | 593 | 4 | −1.3116 | 0.99643 | 0.9965 | 1 | 17241 | 0 | −1.3116 |
| UBR5 | 4 | 0.00045534 | 0.0016412 | 0.049713 | 594 | 4 | −1.3884 | 0.99954 | 0.99957 | 1 | 17632 | 0 | −1.3884 |
| XRCC5 | 4 | 0.00046208 | 0.0016637 | 0.050242 | 595 | 4 | −1.6679 | 0.98681 | 0.98688 | 1 | 16787 | 0 | −1.6679 |
| MED11 | 4 | 0.00046224 | 0.0016642 | 0.050242 | 596 | 4 | −1.4228 | 0.99954 | 0.99956 | 1 | 17628 | 0 | −1.4228 |
| OGT | 4 | 0.00046356 | 0.0016708 | 0.050356 | 597 | 4 | −1.3076 | 0.99938 | 0.9994 | 1 | 17582 | 0 | −1.3076 |
| FZD9 | 4 | 0.00046483 | 0.0016769 | 0.050454 | 598 | 3 | −1.077 | 0.6438 | 0.74784 | 1 | 12454 | 1 | −1.077 |
| IDI1 | 4 | 0.00046651 | 0.0016865 | 0.050658 | 599 | 2 | −0.70394 | 0.12476 | 0.26316 | 0.933374 | 5029 | 2 | −0.70394 |
| KANSL3 | 4 | 0.00047085 | 0.0017015 | 0.050942 | 600 | 4 | −1.734 | 0.99953 | 0.99955 | 1 | 17625 | 0 | −1.734 |
| HEATR2 | 4 | 0.00047103 | 0.0017015 | 0.050942 | 601 | 4 | −0.69545 | 0.99953 | 0.99955 | 1 | 17624 | 0 | −0.69545 |
| NPLOC4 | 4 | 0.00047976 | 0.0017339 | 0.051739 | 602 | 4 | −1.2039 | 0.99902 | 0.999 | 1 | 17496 | 0 | −1.2039 |
| RPL18A | 3 | 0.00048043 | 0.0014388 | 0.045667 | 603 | 3 | −1.5316 | 0.99952 | 0.99952 | 1 | 17622 | 0 | −1.5316 |
| FSCN1 | 4 | 0.0004886 | 0.001758 | 0.052372 | 604 | 4 | −0.94318 | 0.99951 | 0.99953 | 1 | 17621 | 0 | −0.94318 |
| IMP4 | 4 | 0.00049101 | 0.0017706 | 0.052661 | 605 | 4 | −1.8118 | 0.99951 | 0.99953 | 1 | 17620 | 0 | −1.8118 |
| ADSS | 4 | 0.00049411 | 0.0017838 | 0.052965 | 606 | 4 | −1.8117 | 0.99744 | 0.99748 | 1 | 17319 | 0 | −1.8117 |
| NEDD8 | 3 | 0.00049435 | 0.0014706 | 0.046093 | 607 | 2 | −2.4196 | 0.29632 | 0.4365 | 0.994501 | 8406 | 1 | −2.4196 |
| NDUFS5 | 4 | 0.00049625 | 0.0017904 | 0.053074 | 608 | 4 | −0.85767 | 0.9995 | 0.99952 | 1 | 17619 | 0 | −0.85767 |
| GEMIN4 | 4 | 0.00049822 | 0.0018003 | 0.053279 | 609 | 3 | −1.3849 | 0.93909 | 0.93897 | 1 | 15718 | 0 | −1.3849 |
| VPS72 | 4 | 0.00050437 | 0.0018211 | 0.053807 | 610 | 4 | −0.54969 | 0.9995 | 0.99951 | 1 | 17618 | 0 | −0.54969 |
| COPS8 | 4 | 0.00050719 | 0.001831 | 0.053931 | 611 | 4 | −0.97443 | 0.99158 | 0.99171 | 1 | 17006 | 0 | −0.97443 |
| FOXRED1 | 4 | 0.00050799 | 0.0018326 | 0.053931 | 612 | 4 | −0.92674 | 0.99949 | 0.99951 | 1 | 17617 | 0 | −0.92674 |
| KIF18A | 4 | 0.00050856 | 0.0018343 | 0.053931 | 613 | 4 | −1.181 | 0.99949 | 0.99951 | 1 | 17616 | 0 | −1.181 |
| DCTN6 | 4 | 0.00051485 | 0.0018578 | 0.054536 | 614 | 4 | −1.5775 | 0.99922 | 0.99921 | 1 | 17546 | 0 | −1.5775 |
| CEBPZ | 4 | 0.00051741 | 0.0018661 | 0.054583 | 615 | 4 | −1.2677 | 0.99948 | 0.9995 | 1 | 17615 | 0 | −1.2677 |
| ARIH1 | 4 | 0.00051877 | 0.0018716 | 0.054583 | 616 | 4 | −1.1405 | 0.99948 | 0.9995 | 1 | 17614 | 0 | −1.1405 |
| HGS | 4 | 0.0005315 | 0.0019143 | 0.055683 | 617 | 4 | −0.9397 | 0.99947 | 0.99949 | 1 | 17612 | 0 | −0.9397 |
| NIP7 | 4 | 0.00053189 | 0.0019154 | 0.055683 | 618 | 4 | −0.88255 | 0.99947 | 0.99949 | 1 | 17611 | 0 | −0.88255 |
| DUT | 4 | 0.00053876 | 0.0019335 | 0.056003 | 619 | 3 | −0.91528 | 0.58193 | 0.72361 | 1 | 12083 | 1 | −0.91528 |
| HNF1B | 4 | 0.00053945 | 0.0019346 | 0.056003 | 620 | 4 | −0.82201 | 0.99946 | 0.99948 | 1 | 17608 | 0 | −0.82201 |
| RBM48 | 4 | 0.00054006 | 0.0019379 | 0.056003 | 621 | 3 | −1.4768 | 0.81491 | 0.83216 | 1 | 13807 | 1 | −1.4768 |
| CCDC115 | 4 | 0.00054085 | 0.0019423 | 0.056003 | 622 | 4 | −1.2834 | 0.99946 | 0.99948 | 1 | 17607 | 0 | −1.2834 |
| FTSJ3 | 4 | 0.00054093 | 0.0019428 | 0.056003 | 623 | 4 | −1.5605 | 0.99906 | 0.99904 | 1 | 17505 | 0 | −1.5605 |
| ABT1 | 4 | 0.00054165 | 0.001945 | 0.056003 | 624 | 4 | −1.0094 | 0.99946 | 0.99948 | 1 | 17606 | 0 | −1.0094 |
| RANBP1 | 4 | 0.00055194 | 0.0019884 | 0.057159 | 625 | 4 | −0.79378 | 0.99945 | 0.99947 | 1 | 17604 | 0 | −0.79378 |
| BCAS2 | 4 | 0.00055418 | 0.0019977 | 0.057336 | 626 | 4 | −0.74363 | 0.99945 | 0.99946 | 1 | 17603 | 0 | −0.74363 |
| UBR4 | 4 | 0.00055663 | 0.0020103 | 0.057606 | 627 | 4 | −1.0947 | 0.99944 | 0.99946 | 1 | 17602 | 0 | −1.0947 |
| SKA2 | 4 | 0.00055827 | 0.002018 | 0.057675 | 628 | 4 | −1.5851 | 0.99944 | 0.99946 | 1 | 17601 | 0 | −1.5851 |
| EXOSC8 | 4 | 0.00055868 | 0.0020191 | 0.057675 | 629 | 4 | −1.0794 | 0.99944 | 0.99946 | 1 | 17600 | 0 | −1.0794 |
| DPH5 | 4 | 0.00055991 | 0.0020251 | 0.057756 | 630 | 4 | −1.0575 | 0.99944 | 0.99946 | 1 | 17599 | 0 | −1.0575 |
| GSG2 | 4 | 0.0005616 | 0.0020344 | 0.05793 | 631 | 3 | −1.139 | 0.96226 | 0.96215 | 1 | 16156 | 0 | −1.139 |
| RPP30 | 4 | 0.00056405 | 0.0020438 | 0.058104 | 632 | 4 | −1.2535 | 0.98187 | 0.98195 | 1 | 16633 | 0 | −1.2535 |
| RBM10 | 4 | 0.00056735 | 0.0020597 | 0.058343 | 633 | 4 | −1.4299 | 0.99943 | 0.99945 | 1 | 17597 | 0 | −1.4299 |
| NME6 | 4 | 0.00056776 | 0.0020619 | 0.058343 | 634 | 4 | −1.1709 | 0.99943 | 0.99945 | 1 | 17596 | 0 | −1.1709 |
| UBC | 4 | 0.00057464 | 0.0020827 | 0.05884 | 635 | 4 | −1.0724 | 0.99943 | 0.99945 | 1 | 17594 | 0 | −1.0724 |
| HSPA8 | 4 | 0.00057641 | 0.0020898 | 0.058873 | 636 | 4 | −2.0457 | 0.99687 | 0.9969 | 1 | 17277 | 0 | −2.0457 |
| PRKRIR | 3 | 0.00057845 | 0.0017076 | 0.051038 | 637 | 2 | −1.3008 | 0.91929 | 0.91937 | 1 | 15323 | 0 | −1.3008 |
| WNK1 | 4 | 0.00058004 | 0.0021057 | 0.059213 | 638 | 4 | −1.0645 | 0.97597 | 0.97599 | 1 | 16462 | 0 | −1.0645 |
| TSR1 | 4 | 0.00058506 | 0.0021233 | 0.059614 | 639 | 4 | −1.335 | 0.99491 | 0.995 | 1 | 17159 | 0 | −1.335 |
| FASTKD5 | 4 | 0.00058873 | 0.0021326 | 0.059783 | 640 | 4 | −0.91031 | 0.99612 | 0.9962 | 1 | 17218 | 0 | −0.91031 |
| PGGT1B | 4 | 0.00059103 | 0.0021392 | 0.059874 | 641 | 4 | −0.82104 | 0.9991 | 0.99909 | 1 | 17514 | 0 | −0.82104 |
| TFB1M | 4 | 0.00059359 | 0.0021502 | 0.060041 | 642 | 3 | −0.97508 | 0.40719 | 0.57054 | 1 | 9778 | 1 | −0.97508 |
| AURKA | 4 | 0.00059416 | 0.0021518 | 0.060041 | 643 | 4 | −1.3711 | 0.99941 | 0.99943 | 1 | 17592 | 0 | −1.3711 |
| DCTN5 | 4 | 0.0005961 | 0.00216 | 0.060178 | 644 | 3 | −1.6239 | 0.4613 | 0.61753 | 1 | 10486 | 1 | −1.6239 |
| NUBP1 | 4 | 0.00060454 | 0.0021918 | 0.060876 | 645 | 4 | −1.2742 | 0.9994 | 0.99942 | 1 | 17591 | 0 | −1.2742 |
| MRPL38 | 4 | 0.00060696 | 0.0022034 | 0.061084 | 646 | 3 | −1.0939 | 0.71018 | 0.7758 | 1 | 12892 | 1 | −1.0939 |
| EIF2B5 | 4 | 0.00060847 | 0.0022061 | 0.061084 | 647 | 4 | −1.2117 | 0.99939 | 0.99941 | 1 | 17589 | 0 | −1.2117 |
| PI4KA | 4 | 0.00061286 | 0.0022258 | 0.061361 | 648 | 4 | −1.1904 | 0.99939 | 0.99941 | 1 | 17588 | 0 | −1.1904 |
| CNOT7 | 4 | 0.00061481 | 0.0022341 | 0.061361 | 649 | 4 | −0.92744 | 0.99126 | 0.99137 | 1 | 16987 | 0 | −0.92744 |
| TBCA | 4 | 0.0006155 | 0.0022346 | 0.061361 | 650 | 4 | −1.4702 | 0.99938 | 0.9994 | 1 | 17586 | 0 | −1.4702 |
| DYNC1I2 | 4 | 0.0006167 | 0.0022396 | 0.061361 | 651 | 4 | −2.041 | 0.99909 | 0.99907 | 1 | 17510 | 0 | −2.041 |
| ATP5B | 4 | 0.00061705 | 0.0022434 | 0.061361 | 652 | 4 | −1.2287 | 0.99938 | 0.9994 | 1 | 17585 | 0 | −1.2287 |
| NSMCE2 | 4 | 0.0006186 | 0.0022445 | 0.061361 | 653 | 3 | −1.577 | 0.83856 | 0.84789 | 1 | 14076 | 1 | −1.577 |
| NOL6 | 4 | 0.00061904 | 0.002245 | 0.061361 | 654 | 4 | −1.0733 | 0.99938 | 0.9994 | 1 | 17584 | 0 | −1.0733 |
| UTP6 | 4 | 0.00061907 | 0.0022456 | 0.061361 | 655 | 4 | −2.6455 | 0.9957 | 0.99578 | 1 | 17198 | 0 | −2.6455 |
| DDX52 | 4 | 0.0006197 | 0.0022467 | 0.061361 | 656 | 4 | −1.1513 | 0.99938 | 0.9994 | 1 | 17583 | 0 | −1.1513 |
| LSM12 | 4 | 0.00062383 | 0.0022626 | 0.061702 | 657 | 3 | −0.72127 | 0.96186 | 0.96174 | 1 | 16146 | 0 | −0.72127 |
| SLC39A14 | 4 | 0.00062909 | 0.0022774 | 0.062012 | 658 | 4 | −0.91353 | 0.98992 | 0.99 | 1 | 16923 | 0 | −0.91353 |
| RABIF | 4 | 0.00063246 | 0.0022917 | 0.062257 | 659 | 4 | −1.3292 | 0.99901 | 0.99899 | 1 | 17495 | 0 | −1.3292 |
| PSMA6 | 4 | 0.00063294 | 0.0022933 | 0.062257 | 660 | 4 | −1.9056 | 0.99829 | 0.99831 | 1 | 17407 | 0 | −1.9056 |
| GTF2F2 | 3 | 0.00063383 | 0.0018677 | 0.054583 | 661 | 3 | −0.9729 | 0.99937 | 0.99938 | 1 | 17581 | 0 | −0.9729 |
| TBP | 4 | 0.0006339 | 0.0022971 | 0.062268 | 662 | 3 | −1.6473 | 0.94563 | 0.94548 | 1 | 15841 | 0 | −1.6473 |
| RPL7 | 3 | 0.00063477 | 0.0018688 | 0.054583 | 663 | 3 | −1.3947 | 0.99937 | 0.99938 | 1 | 17580 | 0 | −1.3947 |
| DPY30 | 4 | 0.00063718 | 0.0023098 | 0.062516 | 664 | 4 | −0.78669 | 0.99936 | 0.99938 | 1 | 17579 | 0 | −0.78669 |
| GTF3C1 | 4 | 0.00064019 | 0.0023224 | 0.062763 | 665 | 4 | −1.0865 | 0.99516 | 0.99525 | 1 | 17170 | 0 | −1.0865 |
| MYBL2 | 4 | 0.00064309 | 0.0023333 | 0.062965 | 666 | 4 | −0.62528 | 0.99936 | 0.99937 | 1 | 17578 | 0 | −0.62528 |
| TARDBP | 4 | 0.00064652 | 0.0023427 | 0.063066 | 667 | 3 | −1.4541 | 0.91769 | 0.91748 | 1 | 15294 | 0 | −1.4541 |
| ZCCHC9 | 4 | 0.00064896 | 0.0023547 | 0.063066 | 668 | 3 | −1.957 | 0.8992 | 0.89887 | 1 | 14945 | 0 | −1.957 |
| DDOST | 4 | 0.00064905 | 0.0023553 | 0.063066 | 669 | 4 | −0.98102 | 0.99627 | 0.99635 | 1 | 17229 | 0 | −0.98102 |
| NLE1 | 4 | 0.00064926 | 0.0023558 | 0.063066 | 670 | 4 | −1.8629 | 0.99935 | 0.99937 | 1 | 17576 | 0 | −1.8629 |
| COX11 | 4 | 0.00065018 | 0.0023575 | 0.063066 | 671 | 4 | −0.66785 | 0.99935 | 0.99937 | 1 | 17575 | 0 | −0.66785 |
| CSNK2B | 4 | 0.00065043 | 0.002358 | 0.063066 | 672 | 4 | −1.5191 | 0.99791 | 0.99794 | 1 | 17367 | 0 | −1.5191 |
| MRPL50 | 4 | 0.00065289 | 0.0023717 | 0.063339 | 673 | 3 | −1.0478 | 0.92534 | 0.92511 | 1 | 15441 | 0 | −1.0478 |
| COPS5 | 4 | 0.0006578 | 0.0023893 | 0.063598 | 674 | 4 | −1.8379 | 0.99934 | 0.99936 | 1 | 17574 | 0 | −1.8379 |
| SPPL3 | 4 | 0.0006585 | 0.0023909 | 0.063598 | 675 | 4 | −0.79426 | 0.99934 | 0.99936 | 1 | 17573 | 0 | −0.79426 |
| SCO1 | 4 | 0.00065896 | 0.002392 | 0.063598 | 676 | 4 | −0.8189 | 0.99934 | 0.99936 | 1 | 17572 | 0 | −0.8189 |
| POP7 | 4 | 0.00066015 | 0.0023986 | 0.063673 | 677 | 3 | −1.4588 | 0.6613 | 0.75474 | 1 | 12570 | 1 | −1.4588 |
| TEAD1 | 4 | 0.00066078 | 0.0024019 | 0.063673 | 678 | 4 | −0.95911 | 0.99757 | 0.9976 | 1 | 17331 | 0 | −0.95911 |
| RSU1 | 4 | 0.00066227 | 0.0024063 | 0.063696 | 679 | 4 | −0.99983 | 0.98957 | 0.98967 | 1 | 16904 | 0 | −0.99983 |
| TUBB | 4 | 0.00066426 | 0.002414 | 0.063806 | 680 | 4 | −0.95025 | 0.9993 | 0.99932 | 1 | 17564 | 0 | −0.95025 |
| ATP5D | 4 | 0.00066549 | 0.0024183 | 0.063828 | 681 | 4 | −1.02 | 0.99933 | 0.99935 | 1 | 17569 | 0 | −1.02 |
| PSMG2 | 4 | 0.00067324 | 0.0024485 | 0.064522 | 682 | 3 | −1.0601 | 0.85772 | 0.86223 | 1 | 14307 | 1 | −1.0601 |
| THOC5 | 4 | 0.00067466 | 0.0024518 | 0.064522 | 683 | 4 | −0.99515 | 0.99933 | 0.99934 | 1 | 17568 | 0 | −0.99515 |
| UBA2 | 4 | 0.00067928 | 0.0024721 | 0.064882 | 684 | 4 | −1.3364 | 0.99785 | 0.99788 | 1 | 17358 | 0 | −1.3364 |
| THOC3 | 4 | 0.00067939 | 0.0024726 | 0.064882 | 685 | 4 | −1.5118 | 0.99932 | 0.99934 | 1 | 17567 | 0 | −1.5118 |
| PAK1IP1 | 4 | 0.0006813 | 0.0024814 | 0.065017 | 686 | 4 | −1.3674 | 0.99872 | 0.99872 | 1 | 17456 | 0 | −1.3674 |
| VBP1 | 4 | 0.00068655 | 0.0025017 | 0.065454 | 687 | 4 | −0.62699 | 0.99931 | 0.99933 | 1 | 17566 | 0 | −0.62699 |
| SUPT16H | 4 | 0.00068775 | 0.0025083 | 0.065531 | 688 | 4 | −1.2952 | 0.99931 | 0.99933 | 1 | 17565 | 0 | −1.2952 |
| METTL14 | 4 | 0.00069197 | 0.0025215 | 0.065749 | 689 | 3 | −1.2927 | 0.94258 | 0.94242 | 1 | 15783 | 0 | −1.2927 |
| CLP1 | 4 | 0.00069299 | 0.0025239 | 0.065749 | 690 | 4 | −1.2247 | 0.98005 | 0.98014 | 1 | 16585 | 0 | −1.2247 |
| SUPT4H1 | 4 | 0.00070068 | 0.0025511 | 0.066361 | 691 | 3 | −1.3482 | 0.21286 | 0.39886 | 0.981445 | 7252 | 1 | −1.3482 |
| LSM5 | 3 | 0.00070407 | 0.002058 | 0.058343 | 692 | 3 | −2.1031 | 0.9993 | 0.9993 | 1 | 17561 | 0 | −2.1031 |
| NOL8 | 4 | 0.00071105 | 0.0025884 | 0.067223 | 693 | 4 | −0.70785 | 0.99929 | 0.9993 | 1 | 17560 | 0 | −0.70785 |
| RABGGTA | 4 | 0.00071191 | 0.0025917 | 0.067223 | 694 | 4 | −1.3303 | 0.97447 | 0.97444 | 1 | 16432 | 0 | −1.3303 |
| SOD1 | 4 | 0.00071467 | 0.0025982 | 0.067243 | 695 | 3 | −1.4358 | 0.90707 | 0.9068 | 1 | 15107 | 0 | −1.4358 |
| UTP20 | 4 | 0.0007152 | 0.0025999 | 0.067243 | 696 | 4 | −1.7086 | 0.99503 | 0.99512 | 1 | 17163 | 0 | −1.7086 |
| JMJD6 | 4 | 0.00071833 | 0.0026092 | 0.067342 | 697 | 4 | −0.92287 | 0.98666 | 0.98672 | 1 | 16782 | 0 | −0.92287 |
| NDUFS1 | 4 | 0.00071938 | 0.0026136 | 0.067342 | 698 | 4 | −1.3679 | 0.99768 | 0.9977 | 1 | 17345 | 0 | −1.3679 |
| ASH2L | 4 | 0.00072095 | 0.0026196 | 0.067342 | 699 | 4 | −1.0261 | 0.9811 | 0.98117 | 1 | 16612 | 0 | −1.0261 |
| ZBTB11 | 4 | 0.00072143 | 0.0026218 | 0.067342 | 700 | 4 | −0.93541 | 0.99928 | 0.99928 | 1 | 17559 | 0 | −0.93541 |
| MTHFD1 | 4 | 0.00072168 | 0.0026224 | 0.067342 | 701 | 4 | −0.98601 | 0.99928 | 0.99928 | 1 | 17558 | 0 | −0.98601 |
| MRPS2 | 4 | 0.00072305 | 0.0026268 | 0.067359 | 702 | 4 | −1.2672 | 0.99913 | 0.99912 | 1 | 17525 | 0 | −1.2672 |
| PRPF19 | 4 | 0.00073117 | 0.0026547 | 0.06798 | 703 | 4 | −1.5224 | 0.99927 | 0.99927 | 1 | 17557 | 0 | −1.5224 |
| CTNNBL1 | 4 | 0.00073243 | 0.0026621 | 0.068073 | 704 | 4 | −0.96181 | 0.99927 | 0.99927 | 1 | 17556 | 0 | −0.96181 |
| ADSL | 4 | 0.00073946 | 0.0026865 | 0.068503 | 705 | 4 | −1.5065 | 0.9936 | 0.99371 | 1 | 17108 | 0 | −1.5065 |
| NDUFB9 | 4 | 0.00073949 | 0.0026865 | 0.068503 | 706 | 4 | −1.2146 | 0.99926 | 0.99926 | 1 | 17554 | 0 | −1.2146 |
| TRIT1 | 4 | 0.00074457 | 0.0026997 | 0.068741 | 707 | 4 | −1.3456 | 0.99926 | 0.99926 | 1 | 17553 | 0 | −1.3456 |
| PNN | 4 | 0.00074857 | 0.0027123 | 0.068841 | 708 | 4 | −1.0483 | 0.98735 | 0.98743 | 1 | 16822 | 0 | −1.0483 |
| FDX1L | 4 | 0.00074872 | 0.0027123 | 0.068841 | 709 | 4 | −1.0909 | 0.99838 | 0.9984 | 1 | 17421 | 0 | −1.0909 |
| CCDC174 | 4 | 0.00074993 | 0.0027151 | 0.068841 | 710 | 4 | −0.74417 | 0.99925 | 0.99925 | 1 | 17552 | 0 | −0.74417 |
| MRPL11 | 4 | 0.0007518 | 0.0027216 | 0.068911 | 711 | 4 | −1.469 | 0.97559 | 0.97557 | 1 | 16451 | 0 | −1.469 |
| MOGAT3 | 4 | 0.00075403 | 0.0027288 | 0.068995 | 712 | 4 | −0.66172 | 0.99925 | 0.99925 | 1 | 17551 | 0 | −0.66172 |
| TYMS | 4 | 0.00075666 | 0.0027381 | 0.069134 | 713 | 3 | −1.1986 | 0.75574 | 0.79812 | 1 | 13251 | 1 | −1.1986 |
| C16orf59 | 4 | 0.00075866 | 0.0027452 | 0.069217 | 714 | 4 | −0.61374 | 0.99924 | 0.99925 | 1 | 17550 | 0 | −0.61374 |
| PSMB4 | 4 | 0.000768 | 0.0027792 | 0.069893 | 715 | 4 | −0.86869 | 0.99923 | 0.99923 | 1 | 17548 | 0 | −0.86869 |
| MBTPS1 | 4 | 0.00076809 | 0.0027798 | 0.069893 | 716 | 4 | −1.4183 | 0.9986 | 0.99861 | 1 | 17438 | 0 | −1.4183 |
| WDR18 | 4 | 0.00077082 | 0.0027869 | 0.069975 | 717 | 4 | −1.9484 | 0.98937 | 0.98948 | 1 | 16893 | 0 | −1.9484 |
| VMA21 | 4 | 0.00077296 | 0.0027924 | 0.070015 | 718 | 4 | −0.57767 | 0.99923 | 0.99922 | 1 | 17547 | 0 | −0.57767 |
| FXN | 4 | 0.00077503 | 0.0028017 | 0.070151 | 719 | 4 | −1.8392 | 0.99897 | 0.99896 | 1 | 17491 | 0 | −1.8392 |
| UBTF | 4 | 0.00078533 | 0.0028439 | 0.07111 | 720 | 4 | −1.0518 | 0.99921 | 0.99921 | 1 | 17545 | 0 | −1.0518 |
| POLD2 | 4 | 0.00078961 | 0.0028615 | 0.07145 | 721 | 4 | −1.5723 | 0.99031 | 0.9904 | 1 | 16940 | 0 | −1.5723 |
| TIMM13 | 4 | 0.0007917 | 0.0028675 | 0.071502 | 722 | 4 | −0.67054 | 0.99921 | 0.9992 | 1 | 17543 | 0 | −0.67054 |
| TFDP1 | 4 | 0.00079575 | 0.0028829 | 0.071786 | 723 | 3 | −1.7015 | 0.81413 | 0.83167 | 1 | 13792 | 1 | −1.7015 |
| WRAP53 | 4 | 0.00079839 | 0.0028944 | 0.071973 | 724 | 4 | −0.664 | 0.9992 | 0.99919 | 1 | 17542 | 0 | −0.664 |
| TTC27 | 4 | 0.00080971 | 0.002935 | 0.072882 | 725 | 4 | −0.92082 | 0.99919 | 0.99918 | 1 | 17540 | 0 | −0.92082 |
| IQGAP3 | 4 | 0.0008176 | 0.0029662 | 0.073557 | 726 | 4 | −0.5901 | 0.99918 | 0.99917 | 1 | 17539 | 0 | −0.5901 |
| RNF168 | 4 | 0.00082463 | 0.002992 | 0.073846 | 727 | 3 | −0.85943 | 0.64168 | 0.74702 | 1 | 12442 | 1 | −0.85943 |
| RFC2 | 4 | 0.00082635 | 0.002997 | 0.073846 | 728 | 4 | −1.4381 | 0.99353 | 0.99363 | 1 | 17104 | 0 | −1.4381 |
| C12orf45 | 4 | 0.00082719 | 0.0030002 | 0.073846 | 729 | 4 | −1.1627 | 0.99917 | 0.99916 | 1 | 17537 | 0 | −1.1627 |
| RPS2 | 4 | 0.00082747 | 0.0030008 | 0.073846 | 730 | 4 | −1.1531 | 0.99917 | 0.99916 | 1 | 17536 | 0 | −1.1531 |
| KBTBD2 | 4 | 0.00082774 | 0.0030013 | 0.073846 | 731 | 4 | −0.61954 | 0.99917 | 0.99916 | 1 | 17535 | 0 | −0.61954 |
| UBE2I | 4 | 0.00082807 | 0.0030024 | 0.073846 | 732 | 4 | −1.8572 | 0.99788 | 0.99791 | 1 | 17365 | 0 | −1.8572 |
| C7orf26 | 4 | 0.00083133 | 0.003014 | 0.073915 | 733 | 4 | −0.97875 | 0.99917 | 0.99915 | 1 | 17534 | 0 | −0.97875 |
| TTI1 | 4 | 0.00083188 | 0.003015 | 0.073915 | 734 | 4 | −0.99282 | 0.99917 | 0.99915 | 1 | 17533 | 0 | −0.99282 |
| KRAS | 4 | 0.00083267 | 0.0030178 | 0.073915 | 735 | 3 | −1.2216 | 0.83855 | 0.84788 | 1 | 14075 | 1 | −1.2216 |
| ILF3 | 4 | 0.00083382 | 0.0030216 | 0.073915 | 736 | 4 | −1.1038 | 0.99917 | 0.99915 | 1 | 17532 | 0 | −1.1038 |
| GTPBP4 | 4 | 0.00083614 | 0.0030299 | 0.074004 | 737 | 3 | −1.3919 | 0.96604 | 0.96593 | 1 | 16241 | 0 | −1.3919 |
| MRRF | 4 | 0.00083787 | 0.0030359 | 0.074004 | 738 | 3 | −1.2135 | 0.97017 | 0.97009 | 1 | 16328 | 0 | −1.2135 |
| RBX1 | 4 | 0.00083826 | 0.0030375 | 0.074004 | 739 | 4 | −1.1236 | 0.99916 | 0.99915 | 1 | 17531 | 0 | −1.1236 |
| LSM3 | 4 | 0.00084871 | 0.0030798 | 0.074823 | 740 | 4 | −1.5364 | 0.99446 | 0.99456 | 1 | 17144 | 0 | −1.5364 |
| MTERFD2 | 4 | 0.00085056 | 0.0030836 | 0.074823 | 741 | 4 | −1.0458 | 0.99915 | 0.99914 | 1 | 17529 | 0 | −1.0458 |
| POLR3A | 4 | 0.0008536 | 0.0030935 | 0.074894 | 742 | 3 | −2.0153 | 0.97066 | 0.97059 | 1 | 16344 | 0 | −2.0153 |
| GARS | 4 | 0.00085418 | 0.0030962 | 0.074894 | 743 | 3 | −1.6189 | 0.96256 | 0.96246 | 1 | 16161 | 0 | −1.6189 |
| NRF1 | 4 | 0.00085477 | 0.003099 | 0.074894 | 744 | 3 | −1.3049 | 0.93394 | 0.93371 | 1 | 15604 | 0 | −1.3049 |
| TUT1 | 4 | 0.00086187 | 0.0031264 | 0.075455 | 745 | 4 | −1.2081 | 0.99914 | 0.99912 | 1 | 17528 | 0 | −1.2081 |
| C17orf70 | 4 | 0.00086357 | 0.0031346 | 0.075553 | 746 | 4 | −0.7235 | 0.99914 | 0.99912 | 1 | 17527 | 0 | −0.7235 |
| ANAPC2 | 4 | 0.00086813 | 0.0031527 | 0.075739 | 747 | 4 | −1.1956 | 0.99913 | 0.99912 | 1 | 17526 | 0 | −1.1956 |
| IPO13 | 4 | 0.00086892 | 0.0031549 | 0.075739 | 748 | 3 | −1.2412 | 0.4756 | 0.63001 | 1 | 10664 | 1 | −1.2412 |
| EXOC5 | 4 | 0.00087242 | 0.0031659 | 0.07584 | 749 | 4 | −0.81272 | 0.99913 | 0.99912 | 1 | 17524 | 0 | −0.81272 |
| DR1 | 4 | 0.00087266 | 0.0031675 | 0.07584 | 750 | 3 | −0.89635 | 0.55371 | 0.69862 | 1 | 11691 | 1 | −0.89635 |
| SMARCB1 | 4 | 0.00087644 | 0.0031823 | 0.076045 | 751 | 4 | −0.89045 | 0.99912 | 0.99911 | 1 | 17522 | 0 | −0.89045 |
| MCM4 | 4 | 0.00087701 | 0.0031845 | 0.076045 | 752 | 4 | −0.9232 | 0.99912 | 0.99911 | 1 | 17521 | 0 | −0.9232 |
| C16orf80 | 4 | 0.00087904 | 0.0031889 | 0.076045 | 753 | 3 | −1.4216 | 0.96948 | 0.96939 | 1 | 16308 | 0 | −1.4216 |
| XPR1 | 4 | 0.00088105 | 0.0031966 | 0.076045 | 754 | 4 | −1.5392 | 0.99912 | 0.99911 | 1 | 17520 | 0 | −1.5392 |
| ATP6V1D | 4 | 0.00088134 | 0.0031971 | 0.076045 | 755 | 4 | −0.92188 | 0.99912 | 0.99911 | 1 | 17519 | 0 | −0.92188 |
| DGCR8 | 4 | 0.00088323 | 0.0032032 | 0.076088 | 756 | 4 | −1.2954 | 0.97303 | 0.97299 | 1 | 16406 | 0 | −1.2954 |
| TUBE1 | 4 | 0.00088742 | 0.0032207 | 0.076404 | 757 | 4 | −0.97618 | 0.99911 | 0.9991 | 1 | 17518 | 0 | −0.97618 |
| EIF2B1 | 4 | 0.00089062 | 0.0032317 | 0.076476 | 758 | 4 | −0.88744 | 0.99911 | 0.9991 | 1 | 17517 | 0 | −0.88744 |
| DCTN1 | 4 | 0.00089083 | 0.0032322 | 0.076476 | 759 | 3 | −1.2137 | 0.71548 | 0.77824 | 1 | 12928 | 1 | −1.2137 |
| SASS6 | 4 | 0.00089324 | 0.0032421 | 0.076522 | 760 | 4 | −0.90773 | 0.99911 | 0.99909 | 1 | 17516 | 0 | −0.90773 |
| NAA10 | 4 | 0.00089346 | 0.0032427 | 0.076522 | 761 | 4 | −0.97731 | 0.99795 | 0.99799 | 1 | 17373 | 0 | −0.97731 |
| KDM2A | 4 | 0.00089528 | 0.0032498 | 0.07659 | 762 | 4 | −0.97427 | 0.9991 | 0.99909 | 1 | 17515 | 0 | −0.97427 |
| ZNF131 | 4 | 0.0008989 | 0.0032624 | 0.076688 | 763 | 4 | −1.2245 | 0.99667 | 0.99672 | 1 | 17262 | 0 | −1.2245 |
| RNASEH1 | 4 | 0.00090305 | 0.0032788 | 0.076974 | 764 | 4 | −0.99126 | 0.97909 | 0.97918 | 1 | 16554 | 0 | −0.99126 |
| NDUFB10 | 4 | 0.00090741 | 0.0032898 | 0.077083 | 765 | 3 | −1.6413 | 0.89563 | 0.89531 | 1 | 14883 | 1 | −1.6413 |
| FBL | 4 | 0.00090791 | 0.003292 | 0.077083 | 766 | 4 | −0.66521 | 0.99909 | 0.99908 | 1 | 17512 | 0 | −0.66521 |
| HSPA14 | 4 | 0.00091047 | 0.0032986 | 0.077137 | 767 | 4 | −1.7111 | 0.9977 | 0.99772 | 1 | 17346 | 0 | −1.7111 |
| MCAT | 4 | 0.00091414 | 0.0033139 | 0.077396 | 768 | 3 | −1.1744 | 0.972 | 0.97195 | 1 | 16381 | 0 | −1.1744 |
| GINS1 | 4 | 0.00091859 | 0.0033359 | 0.077807 | 769 | 4 | −1.1812 | 0.99908 | 0.99907 | 1 | 17508 | 0 | −1.1812 |
| CENPL | 4 | 0.00092396 | 0.00336 | 0.078269 | 770 | 4 | −1.2548 | 0.99908 | 0.99906 | 1 | 17507 | 0 | −1.2548 |
| NASP | 4 | 0.00092769 | 0.0033726 | 0.078462 | 771 | 4 | −1.4797 | 0.99188 | 0.99201 | 1 | 17019 | 0 | −1.4797 |
| RNPS1 | 4 | 0.00093451 | 0.0033924 | 0.078771 | 772 | 4 | −2.2181 | 0.99505 | 0.99514 | 1 | 17164 | 0 | −2.2181 |
| COX10 | 4 | 0.00093576 | 0.0033979 | 0.078771 | 773 | 4 | −1.6415 | 0.99817 | 0.99822 | 1 | 17395 | 0 | −1.6415 |
| TNPO3 | 4 | 0.00093762 | 0.003405 | 0.078771 | 774 | 4 | −1.1027 | 0.99636 | 0.99643 | 1 | 17238 | 0 | −1.1027 |
| NCAPD2 | 4 | 0.00093871 | 0.0034072 | 0.078771 | 775 | 4 | −1.2175 | 0.99906 | 0.99904 | 1 | 17506 | 0 | −1.2175 |
| ATP6V1E1 | 4 | 0.00093887 | 0.0034077 | 0.078771 | 776 | 4 | −1.1516 | 0.9953 | 0.99539 | 1 | 17180 | 0 | −1.1516 |
| BCCIP | 4 | 0.00094235 | 0.0034198 | 0.078948 | 777 | 4 | −0.67537 | 0.99906 | 0.99904 | 1 | 17504 | 0 | −0.67537 |
| FBXL5 | 4 | 0.00094539 | 0.0034308 | 0.079088 | 778 | 4 | −0.62711 | 0.99905 | 0.99904 | 1 | 17503 | 0 | −0.62711 |
| DDX54 | 4 | 0.0009466 | 0.0034346 | 0.079088 | 779 | 4 | −0.66136 | 0.99905 | 0.99904 | 1 | 17502 | 0 | −0.66136 |
| RPF2 | 4 | 0.00094813 | 0.0034406 | 0.079126 | 780 | 4 | −1.5312 | 0.99905 | 0.99904 | 1 | 17501 | 0 | −1.5312 |
| TMEM130 | 4 | 0.00095179 | 0.0034554 | 0.079277 | 781 | 4 | −0.71549 | 0.99905 | 0.99903 | 1 | 17500 | 0 | −0.71549 |
| PPIL4 | 4 | 0.00095203 | 0.003456 | 0.079277 | 782 | 3 | −1.0547 | 0.29414 | 0.47311 | 1 | 8374 | 1 | −1.0547 |
| OR3A2 | 4 | 0.00095516 | 0.0034675 | 0.079352 | 783 | 4 | −0.63383 | 0.99904 | 0.99903 | 1 | 17499 | 0 | −0.63383 |
| PRPF38B | 4 | 0.00095547 | 0.0034681 | 0.079352 | 784 | 4 | −1.0316 | 0.99904 | 0.99903 | 1 | 17498 | 0 | −1.0316 |
| WDR3 | 4 | 0.00095885 | 0.0034746 | 0.079402 | 785 | 4 | −0.70776 | 0.99904 | 0.99903 | 1 | 17497 | 0 | −0.70776 |
| COPS4 | 4 | 0.0009634 | 0.0034845 | 0.079527 | 786 | 4 | −1.289 | 0.98417 | 0.98423 | 1 | 16700 | 0 | −1.289 |
| ASF1A | 4 | 0.00097743 | 0.0035273 | 0.080325 | 787 | 4 | −1.1002 | 0.99269 | 0.9928 | 1 | 17063 | 0 | −1.1002 |
| WDHD1 | 4 | 0.00097809 | 0.0035284 | 0.080325 | 788 | 4 | −1.3498 | 0.99866 | 0.99868 | 1 | 17446 | 0 | −1.3498 |
| MYBBP1A | 4 | 0.00098661 | 0.0035591 | 0.080923 | 789 | 4 | −1.257 | 0.97931 | 0.97939 | 1 | 16565 | 0 | −1.257 |
| CIRH1A | 4 | 0.00099352 | 0.0035838 | 0.081341 | 790 | 3 | −1.4929 | 0.76107 | 0.80092 | 1 | 13296 | 1 | −1.4929 |
| TXNL4B | 4 | 0.00099482 | 0.0035865 | 0.081341 | 791 | 4 | −1.4073 | 0.97813 | 0.97823 | 1 | 16526 | 0 | −1.4073 |
| MRPL44 | 4 | 0.00099947 | 0.0036013 | 0.081574 | 792 | 4 | −0.64845 | 0.999 | 0.99898 | 1 | 17493 | 0 | −0.64845 |
| RUVBL2 | 4 | 0.001002 | 0.0036139 | 0.081758 | 793 | 4 | −1.1564 | 0.98691 | 0.98699 | 1 | 16800 | 0 | −1.1564 |
| MRPS5 | 4 | 0.0010038 | 0.0036216 | 0.081829 | 794 | 3 | −0.85596 | 0.35766 | 0.52769 | 1 | 9167 | 1 | −0.85596 |
| ROMO1 | 4 | 0.0010124 | 0.0036529 | 0.082251 | 795 | 4 | −1.416 | 0.99645 | 0.99651 | 1 | 17243 | 0 | −1.416 |
| RUVBL1 | 4 | 0.0010129 | 0.003654 | 0.082251 | 796 | 4 | −0.9225 | 0.99899 | 0.99897 | 1 | 17492 | 0 | −0.9225 |
| PGS1 | 4 | 0.0010131 | 0.003654 | 0.082251 | 797 | 4 | −1.1406 | 0.9983 | 0.99833 | 1 | 17410 | 0 | −1.1406 |
| MRPS6 | 4 | 0.0010189 | 0.0036754 | 0.082625 | 798 | 4 | −0.73458 | 0.99045 | 0.99053 | 1 | 16947 | 0 | −0.73458 |
| TCP1 | 4 | 0.0010203 | 0.0036798 | 0.082625 | 799 | 4 | −1.344 | 0.99568 | 0.99577 | 1 | 17197 | 0 | −1.344 |
| OXA1L | 4 | 0.0010309 | 0.0037182 | 0.083384 | 800 | 4 | −1.1411 | 0.9981 | 0.99814 | 1 | 17387 | 0 | −1.1411 |
| XRN2 | 4 | 0.0010368 | 0.0037439 | 0.08379 | 801 | 4 | −1.268 | 0.99896 | 0.99895 | 1 | 17490 | 0 | −1.268 |
| NDUFAF1 | 4 | 0.0010375 | 0.0037456 | 0.08379 | 802 | 3 | −1.3361 | 0.74486 | 0.79249 | 1 | 13171 | 1 | −1.3361 |
| ESF1 | 4 | 0.0010422 | 0.0037615 | 0.084042 | 803 | 4 | −1.2357 | 0.97604 | 0.97606 | 1 | 16464 | 0 | −1.2357 |
| POP5 | 4 | 0.0010453 | 0.0037735 | 0.084207 | 804 | 4 | −1.3235 | 0.99895 | 0.99895 | 1 | 17489 | 0 | −1.3235 |
| TEX10 | 4 | 0.0010539 | 0.0038064 | 0.084837 | 805 | 4 | −1.3169 | 0.99718 | 0.99721 | 1 | 17299 | 0 | −1.3169 |
| C16orf72 | 3 | 0.0010602 | 0.0030682 | 0.074651 | 806 | 3 | −2.2251 | 0.99675 | 0.99679 | 1 | 17266 | 0 | −2.2251 |
| FANCG | 4 | 0.001061 | 0.0038317 | 0.085294 | 807 | 4 | −0.7949 | 0.99579 | 0.99587 | 1 | 17204 | 0 | −0.7949 |
| BUB1B | 4 | 0.0010628 | 0.0038388 | 0.085347 | 808 | 4 | −0.8094 | 0.99894 | 0.99893 | 1 | 17488 | 0 | −0.8094 |
| PDCD7 | 4 | 0.0010671 | 0.003852 | 0.085429 | 809 | 4 | −0.99394 | 0.99689 | 0.99692 | 1 | 17280 | 0 | −0.99394 |
| AP2S1 | 4 | 0.0010671 | 0.003852 | 0.085429 | 810 | 4 | −1.3206 | 0.99236 | 0.99248 | 1 | 17047 | 0 | −1.3206 |
| HNRNPR | 4 | 0.0010775 | 0.0038969 | 0.086321 | 811 | 4 | −0.53381 | 0.99892 | 0.99891 | 1 | 17487 | 0 | −0.53381 |
| KIAA1429 | 4 | 0.0010787 | 0.0039024 | 0.086336 | 812 | 4 | −1.1326 | 0.99722 | 0.99725 | 1 | 17305 | 0 | −1.1326 |
| SNRNP40 | 4 | 0.0010827 | 0.0039156 | 0.086434 | 813 | 4 | −1.1024 | 0.99872 | 0.99872 | 1 | 17454 | 0 | −1.1024 |
| DCLRE1B | 4 | 0.0010846 | 0.0039216 | 0.086434 | 814 | 4 | −0.6801 | 0.99892 | 0.9989 | 1 | 17486 | 0 | −0.6801 |
| ACTB | 3 | 0.0010848 | 0.0031434 | 0.075663 | 815 | 3 | −1.1591 | 0.99892 | 0.99894 | 1 | 17485 | 0 | −1.1591 |
| SMC3 | 4 | 0.0010852 | 0.0039244 | 0.086434 | 816 | 4 | −1.7209 | 0.99891 | 0.9989 | 1 | 17484 | 0 | −1.7209 |
| NDUFC1 | 4 | 0.0010855 | 0.003926 | 0.086434 | 817 | 4 | −0.90005 | 0.99879 | 0.99878 | 1 | 17463 | 0 | −0.90005 |
| USP10 | 4 | 0.00109 | 0.0039381 | 0.086594 | 818 | 4 | −0.90195 | 0.99891 | 0.9989 | 1 | 17483 | 0 | −0.90195 |
| WDR92 | 4 | 0.001094 | 0.0039496 | 0.086742 | 819 | 4 | −1.2556 | 0.99891 | 0.9989 | 1 | 17482 | 0 | −1.2556 |
| DYNC1H1 | 4 | 0.0010951 | 0.0039562 | 0.086781 | 820 | 4 | −1.3605 | 0.99101 | 0.99113 | 1 | 16973 | 0 | −1.3605 |
| POLD3 | 4 | 0.0010964 | 0.0039617 | 0.086787 | 821 | 4 | −1.3598 | 0.9989 | 0.99889 | 1 | 17481 | 0 | −1.3598 |
| PPHLN1 | 4 | 0.0010971 | 0.003966 | 0.086787 | 822 | 4 | −0.829 | 0.9989 | 0.99889 | 1 | 17480 | 0 | −0.829 |
| C1D | 2 | 0.0010976 | 0.0020904 | 0.058873 | 823 | 2 | −1.3624 | 0.9989 | 0.99887 | 1 | 17479 | 0 | −1.3624 |
| CABIN1 | 4 | 0.0011015 | 0.0039852 | 0.087101 | 824 | 4 | −0.78181 | 0.9989 | 0.99889 | 1 | 17478 | 0 | −0.78181 |
| SLC2A1 | 4 | 0.0011039 | 0.0039935 | 0.087118 | 825 | 4 | −0.93683 | 0.9989 | 0.99889 | 1 | 17477 | 0 | −0.93683 |
| MMGT1 | 4 | 0.0011048 | 0.0039957 | 0.087118 | 826 | 4 | −0.74796 | 0.99376 | 0.99386 | 1 | 17114 | 0 | −0.74796 |
| GTPBP8 | 4 | 0.0011153 | 0.0040302 | 0.087765 | 827 | 4 | −1.0746 | 0.99762 | 0.99764 | 1 | 17340 | 0 | −1.0746 |
| TOMM22 | 4 | 0.0011228 | 0.0040549 | 0.088138 | 828 | 4 | −0.82721 | 0.99888 | 0.99887 | 1 | 17475 | 0 | −0.82721 |
| SMC6 | 4 | 0.0011233 | 0.0040571 | 0.088138 | 829 | 4 | −1.1456 | 0.998 | 0.99804 | 1 | 17379 | 0 | −1.1456 |
| EIF5AL1 | 3 | 0.0011297 | 0.003258 | 0.076684 | 830 | 3 | −0.84227 | 0.99887 | 0.9989 | 1 | 17474 | 0 | −0.84227 |
| ALG3 | 4 | 0.0011298 | 0.0040763 | 0.088448 | 831 | 4 | −0.57816 | 0.99887 | 0.99886 | 1 | 17473 | 0 | −0.57816 |
| EIF2S2 | 2 | 0.0011354 | 0.0021666 | 0.060268 | 832 | 2 | −1.5347 | 0.99886 | 0.99883 | 1 | 17472 | 0 | −1.5347 |
| ANAPC15 | 4 | 0.001153 | 0.0041635 | 0.090124 | 833 | 3 | −1.2923 | 0.88586 | 0.88593 | 1 | 14719 | 1 | −1.2923 |
| TTI2 | 4 | 0.0011612 | 0.0041986 | 0.090775 | 834 | 3 | −0.85544 | 0.80587 | 0.82652 | 1 | 13697 | 1 | −0.85544 |
| ARID4B | 4 | 0.0011668 | 0.0042145 | 0.09101 | 835 | 4 | −0.91428 | 0.99883 | 0.99882 | 1 | 17471 | 0 | −0.91428 |
| RPL3 | 4 | 0.0011696 | 0.0042249 | 0.091126 | 836 | 4 | −1.3237 | 0.99883 | 0.99882 | 1 | 17470 | 0 | −1.3237 |
| NOP16 | 4 | 0.0011725 | 0.0042364 | 0.091178 | 837 | 4 | −1.0035 | 0.99883 | 0.99882 | 1 | 17469 | 0 | −1.0035 |
| PWP2 | 4 | 0.0011742 | 0.0042419 | 0.091178 | 838 | 3 | −1.0599 | 0.81627 | 0.83304 | 1 | 13828 | 1 | −1.0599 |
| CS | 4 | 0.0011746 | 0.0042425 | 0.091178 | 839 | 4 | −1.0352 | 0.99883 | 0.99881 | 1 | 17468 | 0 | −1.0352 |
| PEAR1 | 4 | 0.0011768 | 0.0042496 | 0.091222 | 840 | 4 | −0.6471 | 0.99882 | 0.99881 | 1 | 17467 | 0 | −0.6471 |
| NDUFB2 | 4 | 0.0011843 | 0.0042737 | 0.091546 | 841 | 4 | −0.8331 | 0.99882 | 0.9988 | 1 | 17466 | 0 | −0.8331 |
| PDE12 | 4 | 0.0011851 | 0.0042748 | 0.091546 | 842 | 4 | −0.82476 | 0.99712 | 0.99715 | 1 | 17294 | 0 | −0.82476 |
| TRAPPC4 | 4 | 0.0011923 | 0.0042979 | 0.09193 | 843 | 3 | −1.0187 | 0.87581 | 0.87696 | 1 | 14556 | 1 | −1.0187 |
| RIMBP2 | 4 | 0.0011952 | 0.0043094 | 0.092067 | 844 | 4 | −0.4894 | 0.9988 | 0.99879 | 1 | 17465 | 0 | −0.4894 |
| TIFAB | 4 | 0.0012017 | 0.0043357 | 0.09252 | 845 | 4 | −0.6891 | 0.9988 | 0.99879 | 1 | 17464 | 0 | −0.6891 |
| HNRNPL | 4 | 0.0012121 | 0.0043763 | 0.093213 | 846 | 4 | −1.1529 | 0.99298 | 0.99309 | 1 | 17079 | 0 | −1.1529 |
| FNTB | 4 | 0.0012129 | 0.0043785 | 0.093213 | 847 | 4 | −0.98773 | 0.9909 | 0.99101 | 1 | 16963 | 0 | −0.98773 |
| ZNHIT6 | 4 | 0.0012151 | 0.0043883 | 0.093313 | 848 | 3 | −1.4893 | 0.38539 | 0.55164 | 1 | 9525 | 1 | −1.4893 |
| DARS2 | 4 | 0.0012166 | 0.0043938 | 0.09332 | 849 | 4 | −1.095 | 0.99768 | 0.9977 | 1 | 17344 | 0 | −1.095 |
| EIF2S1 | 4 | 0.0012327 | 0.0044629 | 0.094676 | 850 | 3 | −1.3093 | 0.843 | 0.85106 | 1 | 14121 | 1 | −1.3093 |
| LIMS1 | 4 | 0.002348 | 0.0044706 | 0.094728 | 851 | 4 | −0.84075 | 0.99877 | 0.99876 | 1 | 17462 | 0 | −0.84075 |
| WDR12 | 4 | 0.0012445 | 0.0045101 | 0.095352 | 852 | 4 | −1.1622 | 0.99876 | 0.99875 | 1 | 17460 | 0 | −1.1622 |
| POLR2F | 4 | 0.0012449 | 0.0045106 | 0.095352 | 853 | 3 | −1.9506 | 0.93872 | 0.93859 | 1 | 15703 | 0 | −1.9506 |
| MRPL54 | 4 | 0.001266 | 0.0045803 | 0.096598 | 854 | 4 | −0.83933 | 0.99873 | 0.99873 | 1 | 17457 | 0 | −0.83933 |
| BRCA1 | 4 | 0.0012824 | 0.0046324 | 0.097583 | 855 | 4 | −0.6966 | 0.99872 | 0.99872 | 1 | 17455 | 0 | −0.6966 |
| COPS6 | 4 | 0.0012858 | 0.0046472 | 0.097748 | 856 | 4 | −1.1267 | 0.99275 | 0.99288 | 1 | 17068 | 0 | −1.1267 |
| GRWD1 | 4 | 0.0012873 | 0.004651 | 0.097748 | 857 | 3 | −0.90186 | 0.94041 | 0.94028 | 1 | 15742 | 0 | −0.90186 |
| DYNLRB1 | 4 | 0.0012927 | 0.0046642 | 0.09791 | 858 | 4 | −1.1311 | 0.99837 | 0.99839 | 1 | 17417 | 0 | −1.1311 |
| SEZ6 | 4 | 0.0012966 | 0.0046818 | 0.098165 | 859 | 3 | −0.62277 | 0.84297 | 0.85104 | 1 | 14120 | 1 | −0.62277 |
| SMG5 | 4 | 0.0013012 | 0.0047004 | 0.09842 | 860 | 4 | −1.2307 | 0.99035 | 0.99043 | 1 | 16943 | 0 | −1.2307 |
| FANCA | 4 | 0.0013024 | 0.0047064 | 0.09842 | 861 | 4 | −0.68725 | 0.9987 | 0.9987 | 1 | 17453 | 0 | −0.68725 |
| POLR1C | 4 | 0.0013043 | 0.0047136 | 0.09842 | 862 | 4 | −0.96909 | 0.9987 | 0.9987 | 1 | 17452 | 0 | −0.96909 |
| GCN1L1 | 4 | 0.0013059 | 0.0047158 | 0.09842 | 863 | 4 | −0.55419 | 0.99869 | 0.9987 | 1 | 17451 | 0 | −0.55419 |
| DDX49 | 4 | 0.001312 | 0.0047372 | 0.098753 | 864 | 3 | −2.1703 | 0.45827 | 0.61491 | 1 | 10447 | 1 | −2.1703 |
| RMND1 | 4 | 0.0013168 | 0.0047563 | 0.099038 | 865 | 4 | −0.99914 | 0.99868 | 0.9987 | 1 | 17449 | 0 | −0.99914 |
| EIF5B | 4 | 0.0013293 | 0.0047986 | 0.099803 | 866 | 4 | −1.1819 | 0.99867 | 0.99868 | 1 | 17448 | 0 | −1.1819 |
| PPOX | 3 | 0.001464 | 0.0041569 | 0.09009 | 884 | 3 | −0.75081 | 0.99854 | 0.99856 | 1 | 17432 | 0 | −0.75081 |
| RPS19 | 3 | 0.0015856 | 0.0045222 | 0.095484 | 904 | 3 | −2.0213 | 0.99841 | 0.99845 | 1 | 17423 | 0 | −2.0213 |

TABLE 5
Table of shared synthetic lethal genes from TNO-155 CRISPR/Cas9
screen. (Possible drug target enzymes marked in bold text
except surface proteins marked in underlined text.)

| id | H2122-TNO | H2030-TNO | H23-TNO |
|---|---|---|---|
| RTCB | TRUE | FALSE | TRUE |
| ENO1 | TRUE | FALSE | TRUE |
| GAPDH | TRUE | FALSE | TRUE |
| MARS2 | TRUE | FALSE | TRUE |
| ATP6V1F | TRUE | FALSE | TRUE |
| PRMT5 | TRUE | FALSE | TRUE |
| COQ2 | TRUE | FALSE | TRUE |
| DBR1 | TRUE | FALSE | TRUE |
| LYRM4 | TRUE | FALSE | TRUE |
| HIRA | TRUE | FALSE | TRUE |
| RCL1 | TRUE | FALSE | TRUE |
| COA6 | TRUE | FALSE | TRUE |
| WRB | TRUE | FALSE | TRUE |
| TBCB | TRUE | FALSE | TRUE |
| SPATA5 | TRUE | FALSE | TRUE |
| RBBP5 | TRUE | FALSE | TRUE |
| DTYMK | TRUE | FALSE | TRUE |
| GEMIN7 | TRUE | FALSE | TRUE |
| PTPMT1 | TRUE | FALSE | TRUE |
| DKC1 | TRUE | FALSE | TRUE |
| RNMT | TRUE | FALSE | TRUE |
| PPP1R8 | TRUE | FALSE | TRUE |
| HSD17B10 | TRUE | FALSE | TRUE |
| DOLK | TRUE | FALSE | TRUE |
| ALG1 | TRUE | FALSE | TRUE |
| UROD | TRUE | FALSE | TRUE |
| POLR3H | TRUE | FALSE | TRUE |
| LIN52 | TRUE | FALSE | TRUE |
| NAA25 | TRUE | FALSE | TRUE |
| PGD | TRUE | FALSE | TRUE |
| TSEN2 | TRUE | FALSE | TRUE |
| NDUFS2 | TRUE | FALSE | TRUE |
| RNASEH2A | TRUE | FALSE | TRUE |
| GUK1 | TRUE | FALSE | TRUE |
| TSFM | TRUE | FALSE | TRUE |
| MRPL13 | TRUE | FALSE | TRUE |
| WDR77 | TRUE | FALSE | TRUE |
| NELFB | TRUE | FALSE | TRUE |
| DOHH | TRUE | FALSE | TRUE |
| EXOSC5 | TRUE | FALSE | TRUE |
| RPE | TRUE | FALSE | TRUE |
| CSTF1 | TRUE | FALSE | TRUE |
| MRPL53 | TRUE | FALSE | TRUE |
| LSM10 | TRUE | FALSE | TRUE |
| TIMMDC1 | TRUE | FALSE | TRUE |
| RTEL1 | TRUE | FALSE | TRUE |
| PTBP1 | TRUE | TRUE | TRUE |
| WARS2 | TRUE | FALSE | TRUE |
| IBA57 | TRUE | FALSE | TRUE |
| PET117 | TRUE | FALSE | TRUE |
| UTP23 | TRUE | FALSE | TRUE |
| TRAPPC1 | TRUE | FALSE | TRUE |
| MRPS14 | TRUE | FALSE | TRUE |
| POLG2 | TRUE | FALSE | TRUE |
| THG1L | TRUE | FALSE | TRUE |
| RFT1 | TRUE | FALSE | TRUE |
| HSCB | TRUE | FALSE | TRUE |
| EXOSC4 | TRUE | FALSE | TRUE |
| RARS2 | TRUE | FALSE | TRUE |
| MRPL12 | TRUE | FALSE | TRUE |
| ASNA1 | TRUE | FALSE | TRUE |
| CINP | TRUE | FALSE | TRUE |
| RAD51D | TRUE | FALSE | TRUE |
| IMP3 | TRUE | FALSE | TRUE |
| CDC123 | TRUE | FALSE | TRUE |
| MRPS34 | TRUE | FALSE | TRUE |
| NDUFA1 | TRUE | FALSE | TRUE |
| SCO2 | TRUE | FALSE | TRUE |
| RABGGTB | TRUE | FALSE | TRUE |
| ATP5A1 | TRUE | FALSE | TRUE |
| FAM96B | TRUE | FALSE | TRUE |
| LARS2 | TRUE | FALSE | TRUE |
| SDHB | TRUE | FALSE | TRUE |
| ARL2 | TRUE | FALSE | TRUE |
| GCSH | TRUE | FALSE | TRUE |
| COX5B | TRUE | FALSE | TRUE |
| CPSF4 | TRUE | FALSE | TRUE |
| SAMM50 | TRUE | FALSE | TRUE |
| PDPK1 | TRUE | FALSE | TRUE |
| WAPAL | TRUE | FALSE | TRUE |
| PHB | TRUE | FALSE | TRUE |
| NOP9 | TRUE | FALSE | TRUE |
| GTF2A2 | TRUE | FALSE | TRUE |
| DDX10 | TRUE | FALSE | TRUE |
| DNLZ | TRUE | FALSE | TRUE |
| DAP3 | TRUE | FALSE | TRUE |
| SNAPC3 | TRUE | FALSE | TRUE |
| PDCD11 | TRUE | FALSE | TRUE |
| PDCD2 | TRUE | FALSE | TRUE |
| NAA20 | TRUE | FALSE | TRUE |
| GEMIN5 | TRUE | FALSE | TRUE |
| TFRC | TRUE | FALSE | TRUE |
| VARS2 | TRUE | FALSE | TRUE |
| AIFM1 | TRUE | FALSE | TRUE |
| PTCD3 | TRUE | FALSE | TRUE |
| PDSS2 | TRUE | FALSE | TRUE |
| MRPL15 | TRUE | FALSE | TRUE |
| PSMG4 | TRUE | FALSE | TRUE |
| ORAOV1 | TRUE | FALSE | TRUE |
| ACTR6 | TRUE | FALSE | TRUE |
| DHX33 | TRUE | FALSE | TRUE |
| TEN1 | TRUE | FALSE | TRUE |
| COASY | TRUE | FALSE | TRUE |
| VHL | TRUE | FALSE | TRUE |
| NDNL2 | TRUE | FALSE | TRUE |
| TOMM70A | TRUE | FALSE | TRUE |
| DRAP1 | TRUE | FALSE | TRUE |
| NOL9 | TRUE | FALSE | TRUE |
| RNGTT | TRUE | FALSE | TRUE |
| PPP1R2 | TRUE | FALSE | TRUE |
| NOL11 | TRUE | FALSE | TRUE |
| CTDNEP1 | TRUE | FALSE | TRUE |
| TPT1 | TRUE | FALSE | TRUE |
| TIMM44 | TRUE | FALSE | TRUE |
| ISG20L2 | TRUE | FALSE | TRUE |
| PES1 | TRUE | FALSE | TRUE |
| ERCC2 | TRUE | FALSE | TRUE |
| TOP3A | TRUE | FALSE | TRUE |
| MTG2 | TRUE | FALSE | TRUE |
| INTS3 | TRUE | FALSE | TRUE |
| BRF1 | TRUE | FALSE | TRUE |
| PPIL2 | TRUE | FALSE | TRUE |
| PIK3C3 | TRUE | FALSE | TRUE |
| SRP9 | TRUE | FALSE | TRUE |
| MCTS1 | TRUE | FALSE | TRUE |
| TCOF1 | TRUE | FALSE | TRUE |
| MRPL47 | TRUE | FALSE | TRUE |
| COQ6 | TRUE | FALSE | TRUE |
| TUBD1 | TRUE | FALSE | TRUE |
| MRPL28 | TRUE | FALSE | TRUE |
| NSMCE1 | TRUE | FALSE | TRUE |
| NOL10 | TRUE | FALSE | TRUE |
| CCT4 | TRUE | FALSE | TRUE |
| ATP5O | TRUE | FALSE | TRUE |
| XRCC6 | TRUE | FALSE | TRUE |
| SPCS2 | TRUE | FALSE | TRUE |
| URB1 | TRUE | FALSE | TRUE |
| TAF1B | TRUE | FALSE | TRUE |
| DNAJA3 | TRUE | FALSE | TRUE |
| TAF3 | TRUE | FALSE | TRUE |
| WBSCR16 | TRUE | FALSE | TRUE |
| RPN1 | TRUE | FALSE | TRUE |
| MIPEP | TRUE | FALSE | TRUE |
| GTF2H1 | TRUE | FALSE | TRUE |
| IARS | TRUE | FALSE | TRUE |
| CDC37 | TRUE | FALSE | TRUE |
| AURKAIP1 | TRUE | FALSE | TRUE |
| EIF1AD | TRUE | FALSE | TRUE |
| RIOK1 | TRUE | FALSE | TRUE |
| SRP14 | TRUE | FALSE | TRUE |
| UQCRES1 | TRUE | FALSE | TRUE |
| SPATA5L1 | TRUE | FALSE | TRUE |
| EXOSC10 | TRUE | FALSE | TRUE |
| CTC1 | TRUE | FALSE | TRUE |
| PRMT1 | TRUE | FALSE | TRUE |
| DDX59 | TRUE | FALSE | TRUE |
| ILF2 | TRUE | FALSE | TRUE |
| FBXW11 | TRUE | FALSE | TRUE |
| MARS | TRUE | FALSE | TRUE |
| CHTF8 | TRUE | FALSE | TRUE |
| TOE1 | TRUE | FALSE | TRUE |
| MRPL41 | TRUE | FALSE | TRUE |
| GEMIN8 | TRUE | FALSE | TRUE |
| MTX1 | TRUE | FALSE | TRUE |
| TFAM | TRUE | FALSE | TRUE |
| MRPL39 | TRUE | FALSE | TRUE |
| SLC7A6OS | TRUE | FALSE | TRUE |
| ATP6V0C | TRUE | FALSE | TRUE |
| SARS2 | TRUE | FALSE | TRUE |
| CD3EAP | TRUE | FALSE | TRUE |
| NELFA | TRUE | FALSE | TRUE |
| HAUS1 | TRUE | FALSE | TRUE |
| VPS16 | TRUE | FALSE | TRUE |
| ORC2 | TRUE | FALSE | TRUE |
| TRAPPC3 | TRUE | FALSE | TRUE |
| MRPL3 | TRUE | FALSE | TRUE |
| CDIPT | TRUE | FALSE | TRUE |
| YAE1D1 | TRUE | FALSE | TRUE |
| YARS | TRUE | FALSE | TRUE |
| TUBG1 | TRUE | FALSE | TRUE |
| RRP9 | TRUE | FALSE | TRUE |
| CARS2 | TRUE | FALSE | TRUE |
| NUP50 | TRUE | FALSE | TRUE |
| RBM17 | TRUE | FALSE | TRUE |
| MAD2L2 | TRUE | FALSE | TRUE |
| PPP2R4 | TRUE | FALSE | TRUE |
| NHP2 | TRUE | FALSE | TRUE |
| RPS16 | TRUE | FALSE | TRUE |
| OIP5 | TRUE | FALSE | TRUE |
| RPP21 | TRUE | FALSE | TRUE |
| UGP2 | TRUE | FALSE | TRUE |
| MRPL45 | TRUE | FALSE | TRUE |
| DPAGT1 | TRUE | FALSE | TRUE |
| PYROXD1 | TRUE | FALSE | TRUE |
| TIMM10 | TRUE | FALSE | TRUE |
| MTOR | TRUE | FALSE | TRUE |
| HARS2 | TRUE | FALSE | TRUE |
| PELP1 | TRUE | FALSE | TRUE |
| GNB2L1 | TRUE | FALSE | TRUE |
| MRPL37 | TRUE | FALSE | TRUE |
| NARS | TRUE | FALSE | TRUE |
| TSC1 | TRUE | FALSE | TRUE |
| POLR3C | TRUE | FALSE | TRUE |
| SEC63 | TRUE | FALSE | TRUE |
| QRSL1 | TRUE | FALSE | TRUE |
| RPIA | TRUE | FALSE | TRUE |
| THOC7 | TRUE | FALSE | TRUE |
| BUB3 | TRUE | FALSE | TRUE |
| SDHC | TRUE | FALSE | TRUE |
| RIOK2 | TRUE | FALSE | TRUE |
| DDX56 | TRUE | FALSE | TRUE |
| MRPS18C | TRUE | FALSE | TRUE |
| CENPM | TRUE | FALSE | TRUE |
| EIF4E | TRUE | FALSE | TRUE |
| MPHOSPH10 | TRUE | FALSE | TRUE |
| DDX46 | TRUE | FALSE | TRUE |
| IMPDH2 | TRUE | FALSE | TRUE |
| SOD2 | TRUE | FALSE | TRUE |
| UBE2M | TRUE | FALSE | TRUE |
| CIAO1 | TRUE | FALSE | TRUE |
| COQ4 | TRUE | FALSE | TRUE |
| GATC | TRUE | FALSE | TRUE |
| TFB2M | TRUE | FALSE | TRUE |
| TSC2 | TRUE | FALSE | TRUE |
| COX6B1 | TRUE | FALSE | TRUE |
| PMPCA | TRUE | FALSE | TRUE |
| CCT7 | TRUE | FALSE | TRUE |
| WDR61 | TRUE | FALSE | TRUE |
| TSEN54 | TRUE | FALSE | TRUE |
| TOMM40 | TRUE | FALSE | TRUE |
| FOXM1 | TRUE | FALSE | TRUE |
| MRPS18B | TRUE | FALSE | TRUE |
| NDUFA11 | TRUE | FALSE | TRUE |
| COA3 | TRUE | FALSE | TRUE |
| ATP5F1 | TRUE | FALSE | TRUE |
| YRDC | TRUE | FALSE | TRUE |
| FARS2 | TRUE | FALSE | TRUE |
| CTPS1 | TRUE | FALSE | TRUE |
| SNUPN | TRUE | FALSE | TRUE |
| WDR25 | TRUE | FALSE | TRUE |
| PARS2 | TRUE | FALSE | TRUE |
| ELP4 | TRUE | FALSE | TRUE |
| TIMM22 | TRUE | FALSE | TRUE |
| EXOSC7 | TRUE | FALSE | TRUE |
| ALG2 | TRUE | FALSE | TRUE |
| MRPS12 | TRUE | FALSE | TRUE |
| TOMM20 | TRUE | FALSE | TRUE |
| EIF2B3 | TRUE | FALSE | TRUE |
| CMPK1 | TRUE | FALSE | TRUE |
| GFER | TRUE | FALSE | TRUE |
| CHORDC1 | TRUE | FALSE | TRUE |
| XRCC2 | TRUE | FALSE | TRUE |
| CFDP1 | TRUE | FALSE | TRUE |
| DHDDS | TRUE | FALSE | TRUE |
| C10orf2 | TRUE | FALSE | TRUE |
| SAE1 | TRUE | FALSE | TRUE |
| LETM1 | TRUE | FALSE | TRUE |
| PCBP1 | TRUE | FALSE | TRUE |
| DNAJC17 | TRUE | FALSE | TRUE |
| EXOSC9 | TRUE | FALSE | TRUE |
| NARS2 | TRUE | FALSE | TRUE |
| NDOR1 | TRUE | FALSE | TRUE |
| WDR1 | TRUE | FALSE | TRUE |
| TBL3 | TRUE | FALSE | TRUE |
| TNPO1 | TRUE | FALSE | TRUE |
| SELRC1 | TRUE | FALSE | TRUE |
| PNKP | TRUE | FALSE | TRUE |
| CYC1 | TRUE | FALSE | TRUE |
| PDSS1 | TRUE | FALSE | TRUE |
| MRPS35 | TRUE | FALSE | TRUE |
| POLR3K | TRUE | FALSE | TRUE |
| FAM210A | TRUE | FALSE | TRUE |
| NCAPG | TRUE | FALSE | TRUE |
| AHCY | TRUE | FALSE | TRUE |
| NAE1 | TRUE | FALSE | TRUE |
| NDUFAF6 | TRUE | FALSE | TRUE |
| MRPL4 | TRUE | FALSE | TRUE |
| MRPS24 | TRUE | FALSE | TRUE |
| UBIAD1 | TRUE | FALSE | TRUE |
| FDXR | TRUE | FALSE | TRUE |
| RPUSD4 | TRUE | FALSE | TRUE |
| FTSJ2 | TRUE | FALSE | TRUE |
| EARS2 | TRUE | FALSE | TRUE |
| CENPN | TRUE | FALSE | TRUE |
| RAD1 | TRUE | FALSE | TRUE |
| SUGT1 | TRUE | FALSE | TRUE |
| GMPPB | TRUE | FALSE | TRUE |
| GNB1L | TRUE | FALSE | TRUE |
| LIAS | TRUE | FALSE | TRUE |
| ATP2A2 | TRUE | FALSE | TRUE |
| PPP4C | TRUE | FALSE | TRUE |
| ATP6AP2 | TRUE | FALSE | TRUE |
| C19orf52 | TRUE | FALSE | TRUE |
| WDR46 | TRUE | FALSE | TRUE |
| CCT5 | TRUE | FALSE | TRUE |
| NDUFAF4 | TRUE | FALSE | TRUE |
| NAF1 | TRUE | FALSE | TRUE |
| NSUN4 | TRUE | FALSE | TRUE |
| ACTR3 | TRUE | FALSE | TRUE |
| C9orf114 | TRUE | FALSE | TRUE |
| DLD | TRUE | FALSE | TRUE |
| TRMT5 | TRUE | FALSE | TRUE |
| ZNF407 | TRUE | FALSE | TRUE |
| AASDHPPT | TRUE | FALSE | TRUE |
| TSR2 | TRUE | FALSE | TRUE |
| EIF5A | TRUE | FALSE | TRUE |
| STIL | TRUE | FALSE | TRUE |
| POT1 | TRUE | FALSE | TRUE |
| ARMC5 | TRUE | FALSE | TRUE |
| MED8 | TRUE | FALSE | TRUE |
| DSCC1 | TRUE | FALSE | TRUE |
| TCEB2 | TRUE | FALSE | TRUE |
| NSF | TRUE | FALSE | TRUE |
| DHX9 | TRUE | FALSE | TRUE |
| LONP1 | TRUE | FALSE | TRUE |
| PPP6C | TRUE | FALSE | TRUE |
| HNRNPU | TRUE | FALSE | TRUE |
| ELP5 | TRUE | FALSE | TRUE |
| SLC25A19 | TRUE | FALSE | TRUE |
| COX15 | TRUE | FALSE | TRUE |
| SKIV2L2 | TRUE | FALSE | TRUE |
| MRPL35 | TRUE | FALSE | TRUE |
| PTDSS1 | TRUE | FALSE | TRUE |
| USP5 | TRUE | FALSE | TRUE |
| VPS52 | TRUE | FALSE | TRUE |
| LRPPRC | TRUE | FALSE | TRUE |
| C21orf59 | TRUE | FALSE | TRUE |
| TAF1C | TRUE | FALSE | TRUE |
| TKT | TRUE | FALSE | TRUE |
| TRMT61A | TRUE | FALSE | TRUE |
| CHCHD4 | TRUE | FALSE | TRUE |
| RCC1 | TRUE | FALSE | TRUE |
| RPS11 | TRUE | FALSE | TRUE |
| NUDC | TRUE | FALSE | TRUE |
| ARPC4 | TRUE | FALSE | TRUE |
| RTN4IP1 | TRUE | FALSE | TRUE |
| RPS21 | TRUE | FALSE | TRUE |
| N6AMT1 | TRUE | FALSE | TRUE |
| GGPS1 | TRUE | FALSE | TRUE |
| EFTUD1 | TRUE | FALSE | TRUE |
| RPL14 | TRUE | FALSE | TRUE |
| DNAJC9 | TRUE | TRUE | TRUE |
| ACAD9 | TRUE | FALSE | TRUE |
| HUS1 | TRUE | FALSE | TRUE |
| SYS1 | TRUE | FALSE | TRUE |
| CLTC | TRUE | FALSE | TRUE |
| VRK1 | TRUE | FALSE | TRUE |
| SETD1A | TRUE | FALSE | TRUE |
| VMP1 | TRUE | FALSE | TRUE |
| IPO11 | TRUE | FALSE | TRUE |
| MRPL51 | TRUE | FALSE | TRUE |
| MCMBP | TRUE | FALSE | TRUE |
| EIF3I | TRUE | FALSE | TRUE |
| METTL16 | TRUE | FALSE | TRUE |
| MASTL | TRUE | FALSE | TRUE |
| DDX51 | TRUE | FALSE | TRUE |
| CCDC94 | TRUE | FALSE | TRUE |
| MRPL34 | TRUE | FALSE | TRUE |
| SRPRB | TRUE | FALSE | TRUE |
| ATP6V0B | TRUE | FALSE | TRUE |
| AATF | TRUE | FALSE | TRUE |
| ADAT3 | TRUE | FALSE | TRUE |
| UQCRC2 | TRUE | FALSE | TRUE |
| KRR1 | TRUE | FALSE | TRUE |
| ZNRD1 | TRUE | FALSE | TRUE |
| XRCC5 | TRUE | FALSE | TRUE |
| OGT | TRUE | FALSE | TRUE |
| IDI1 | TRUE | FALSE | TRUE |
| IMP4 | TRUE | FALSE | TRUE |
| GEMIN4 | TRUE | FALSE | TRUE |
| FOXRED1 | TRUE | FALSE | TRUE |
| DCTN6 | TRUE | FALSE | TRUE |
| HGS | TRUE | FALSE | TRUE |
| NIP7 | TRUE | FALSE | TRUE |
| CCDC115 | TRUE | FALSE | TRUE |
| FTSJ3 | TRUE | FALSE | TRUE |
| ABT1 | TRUE | FALSE | TRUE |
| BCAS2 | TRUE | FALSE | TRUE |
| UBR4 | TRUE | FALSE | TRUE |
| EXOSC8 | TRUE | FALSE | TRUE |
| GSG2 | TRUE | FALSE | TRUE |
| HSPA8 | TRUE | FALSE | TRUE |
| TSR1 | TRUE | FALSE | TRUE |
| FASTKD5 | TRUE | FALSE | TRUE |
| TFB1M | TRUE | FALSE | TRUE |
| MRPL38 | TRUE | FALSE | TRUE |
| PI4KA | TRUE | FALSE | TRUE |
| TBCA | TRUE | FALSE | TRUE |
| ATP5B | TRUE | FALSE | TRUE |
| NSMCE2 | TRUE | FALSE | TRUE |
| NOL6 | TRUE | FALSE | TRUE |
| DDX52 | TRUE | FALSE | TRUE |
| LSM12 | TRUE | FALSE | TRUE |
| GTF3C1 | TRUE | FALSE | TRUE |
| TARDBP | TRUE | FALSE | TRUE |
| DDOST | TRUE | FALSE | TRUE |
| NLE1 | TRUE | FALSE | TRUE |
| COX11 | TRUE | FALSE | TRUE |
| CSNK2B | TRUE | FALSE | TRUE |
| SCO1 | TRUE | FALSE | TRUE |
| POP7 | TRUE | FALSE | TRUE |
| ATP5D | TRUE | FALSE | TRUE |
| THOC5 | TRUE | FALSE | TRUE |
| UBA2 | TRUE | FALSE | TRUE |
| RABGGTA | TRUE | FALSE | TRUE |
| SOD1 | TRUE | FALSE | TRUE |
| UTP20 | TRUE | FALSE | TRUE |
| MRPS2 | TRUE | FALSE | TRUE |
| CTNNBL1 | TRUE | FALSE | TRUE |
| NDUFB9 | TRUE | FALSE | TRUE |
| TRIT1 | TRUE | FALSE | TRUE |
| FDX1L | TRUE | FALSE | TRUE |
| MRPL11 | TRUE | FALSE | TRUE |
| TYMS | TRUE | FALSE | TRUE |
| C16orf59 | TRUE | FALSE | TRUE |
| WDR18 | TRUE | FALSE | TRUE |
| VMA21 | TRUE | FALSE | TRUE |
| FXN | TRUE | FALSE | TRUE |
| TIMM13 | TRUE | FALSE | TRUE |
| WRAP53 | TRUE | FALSE | TRUE |
| TTC27 | TRUE | FALSE | TRUE |
| RNF168 | TRUE | FALSE | TRUE |
| RFC2 | TRUE | FALSE | TRUE |
| C12orf45 | TRUE | FALSE | TRUE |
| RPS2 | TRUE | FALSE | TRUE |
| UBE2I | TRUE | FALSE | TRUE |
| C7orf26 | TRUE | FALSE | TRUE |
| TTI1 | TRUE | FALSE | TRUE |
| ILF3 | TRUE | TRUE | TRUE |
| MRRF | TRUE | FALSE | TRUE |
| MTERFD2 | TRUE | FALSE | TRUE |
| GARS | TRUE | FALSE | TRUE |
| IPO13 | TRUE | FALSE | TRUE |
| EXOC5 | TRUE | FALSE | TRUE |
| SMARCB1 | TRUE | FALSE | TRUE |
| ATP6V1D | TRUE | FALSE | TRUE |
| DGCR8 | TRUE | FALSE | TRUE |
| TUBE1 | TRUE | FALSE | TRUE |
| EIF2B1 | TRUE | FALSE | TRUE |
| RNASEH1 | TRUE | FALSE | TRUE |
| NDUFB10 | TRUE | FALSE | TRUE |
| FBL | TRUE | FALSE | TRUE |
| MCAT | TRUE | FALSE | TRUE |
| COX10 | TRUE | FALSE | TRUE |
| NCAPD2 | TRUE | FALSE | TRUE |
| ATP6V1E1 | TRUE | FALSE | TRUE |
| BCCIP | TRUE | FALSE | TRUE |
| RPS2 | TRUE | FALSE | TRUE |
| PRPF38B | TRUE | FALSE | TRUE |
| WDR3 | TRUE | FALSE | TRUE |
| MYBBP1A | TRUE | FALSE | TRUE |
| CIRH1A | TRUE | FALSE | TRUE |
| TXNL4B | TRUE | FALSE | TRUE |
| MRPL44 | TRUE | FALSE | TRUE |
| MRPS6 | TRUE | FALSE | TRUE |
| OXA1L | TRUE | FALSE | TRUE |
| XRN2 | TRUE | FALSE | TRUE |
| NDUFAF1 | TRUE | FALSE | TRUE |
| POP5 | TRUE | FALSE | TRUE |
| TEX10 | TRUE | FALSE | TRUE |
| BUB1B | TRUE | FALSE | TRUE |
| AP2S1 | TRUE | FALSE | TRUE |
| SNRNP40 | TRUE | FALSE | TRUE |
| DCLRE1B | TRUE | FALSE | TRUE |
| NDUFC1 | TRUE | FALSE | TRUE |
| WDR92 | TRUE | FALSE | TRUE |
| MMGT1 | TRUE | FALSE | TRUE |
| GTPBP8 | TRUE | FALSE | TRUE |
| TOMM22 | TRUE | FALSE | TRUE |
| SMC6 | TRUE | FALSE | TRUE |
| TTI2 | TRUE | FALSE | TRUE |
| NOP16 | TRUE | FALSE | TRUE |
| PWP2 | TRUE | FALSE | TRUE |
| CS | TRUE | FALSE | TRUE |
| PEAR1 | TRUE | FALSE | TRUE |
| TRAPPC4 | TRUE | FALSE | TRUE |
| HNRNPL | TRUE | FALSE | TRUE |
| FNTB | TRUE | FALSE | TRUE |
| DARS2 | TRUE | FALSE | TRUE |
| WDR12 | TRUE | FALSE | TRUE |
| MRPL54 | TRUE | FALSE | TRUE |
| GRWD1 | TRUE | FALSE | TRUE |
| DYNLRB1 | TRUE | FALSE | TRUE |
| RMND1 | TRUE | FALSE | TRUE |

### Example 5. TEAD Inhibitors Enhance the Efficacy of G12Ci in KRAS Mutant-Driven NSCLC

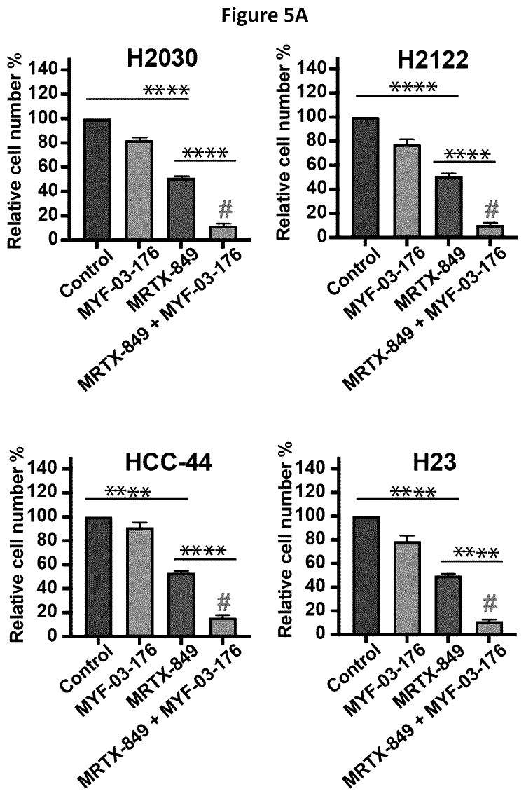

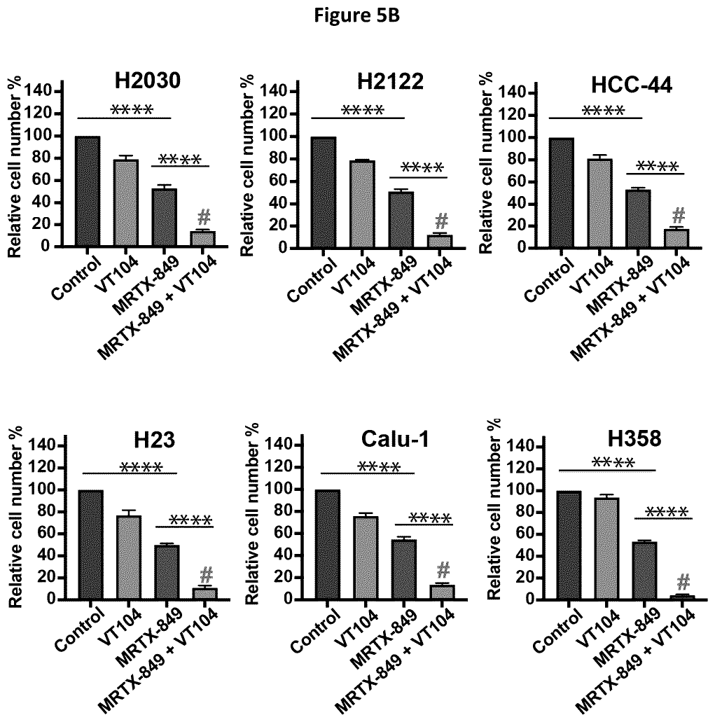

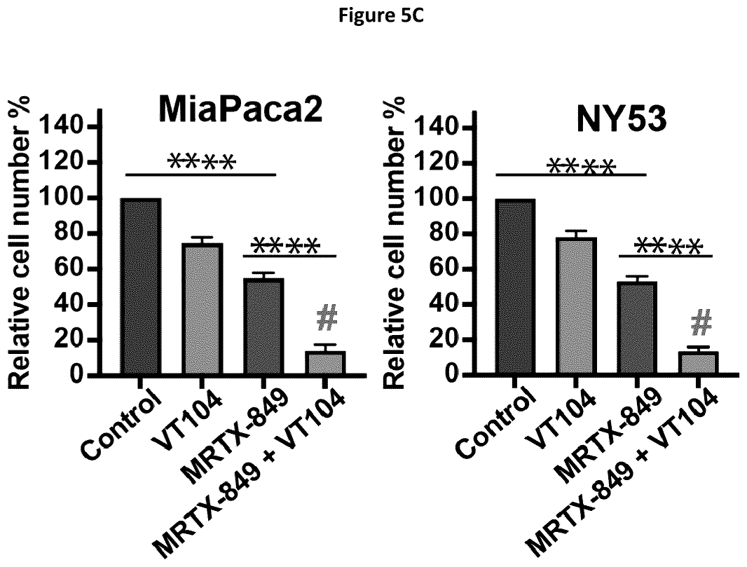

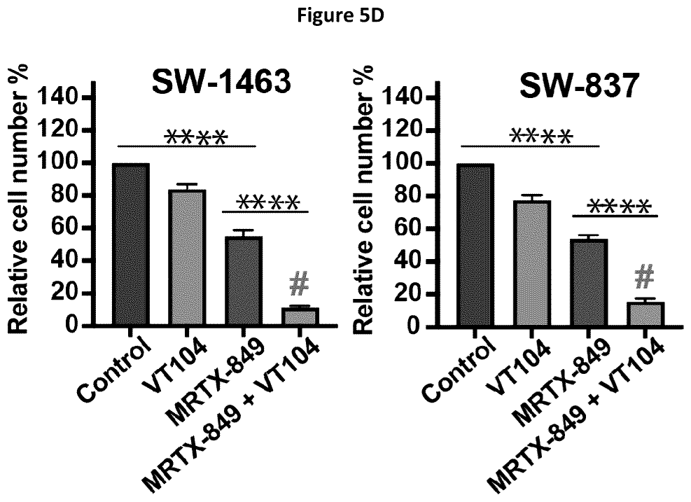

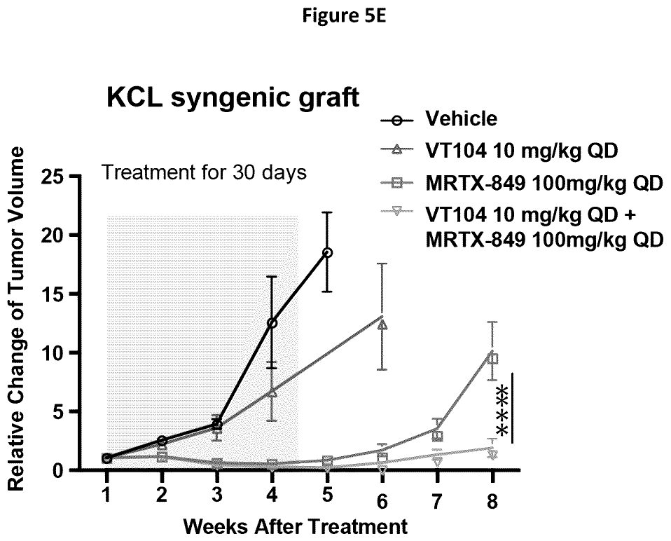

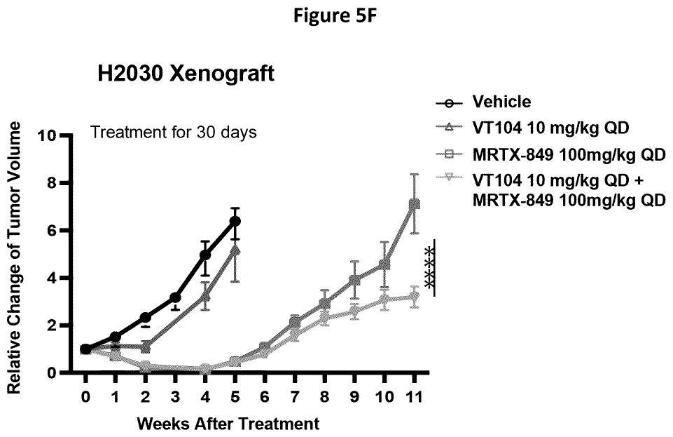

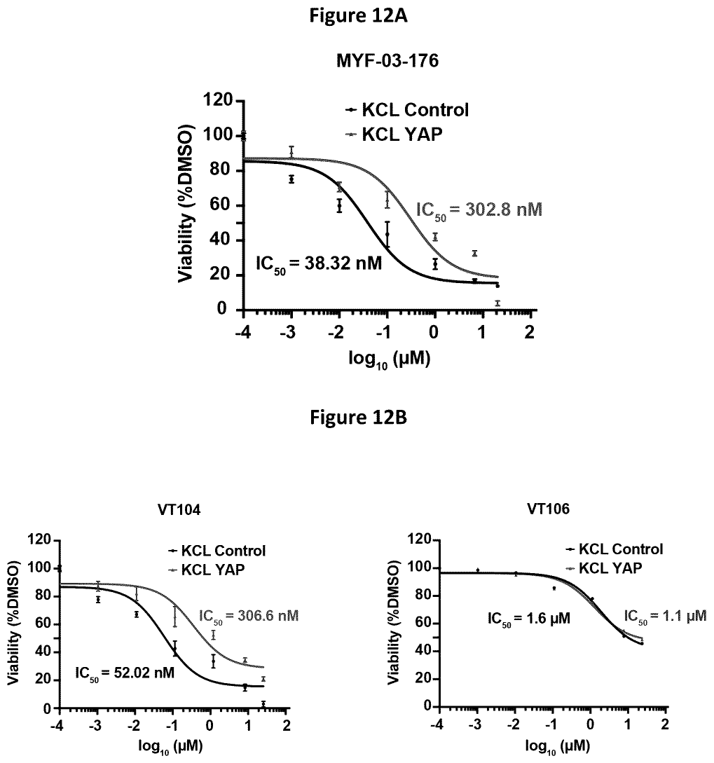

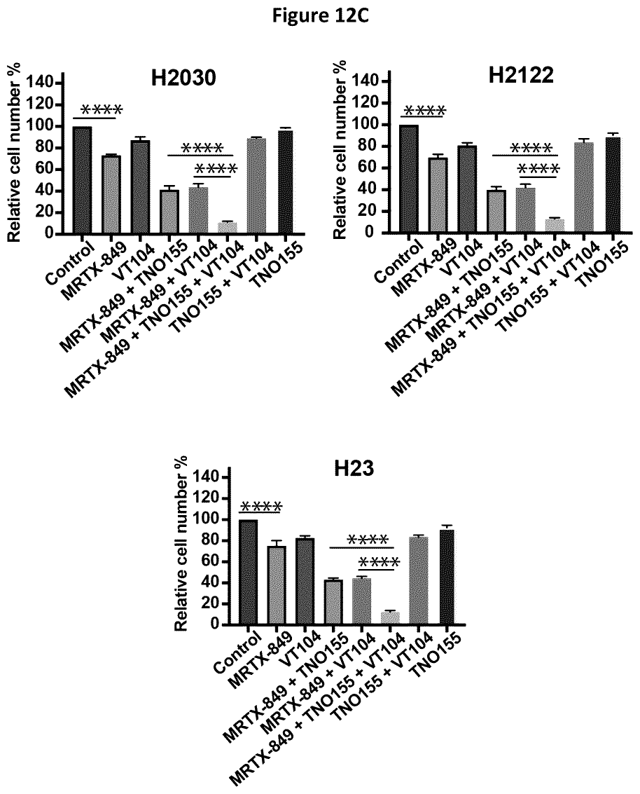

The effects of two mechanistically distinct TEAD inhibitors were tested on MRTX-849 efficacy. MYF-03-69 is a tool compound that binds covalently to the conserved TEAD palmitate pocket, irreversibly disrupting YAP-TEAD association and suppressing TEAD1-4 transcriptional activity (38). VT-104 is a clinical grade, non-covalent pan-TEAD inhibitor that also binds the palmitate pocket. Consistent with the above genetic results, both agents synergistically augmented MRTX-849 action in multiple NSCLC lines, including those that were not STK11 and/or KEAP1mutant (FIGS. 5A and 5B). Notably, this combination was also active in select PDAC and CRC cell lines (FIGS. 5C and 5D). MYF-03-176 and VT-104 each showed higher IC₅₀s (combined with MRTX-849 at its IC₅₀ dose) in YAP1 over-expressing KCL cells, arguing that their effects were on-target (FIGS. 12A and B). Furthermore, VT106, an inactive analog of VT-104 that does not block YAP/TEAD interaction (39), had no effect on drug sensitivity. VT-104 also increased the efficacy of combination MRTX849/TNO-155 in NSCLC lines (FIG. 12C) compared with single agent therapy or two drug-combinations (all at reduced doses).

Given these pre-clinical data, whether TEAD inhibition could enhance MRTX-849 efficacy in mice was tested. At full doses (100 mg/kg/d), MRTX-849 is quite active against KCL syngeneic tumor grafts and H2030 cell-derived xenografts, resulting in complete responses (no detectable tumor) after 30 days of treatment. Some malignant cells remain, however, because after drug withdrawal, tumors recur. VT104 (10 mg/kg/d) has minimal single agent efficacy in either model. However, combining MRTX849 and VT104 results in a significant delay in tumor recurrence in both models (FIGS. 5E and 5F). Collectively, then, combined G12C and TEAD inhibition shows increased efficacy over G12Ci alone in vivo as well as in vitro.

### Example 6. Resistance to G12Ci or G12Ci+SHP2i in Mouse and Human Induces Similar Pathways to Those Targeted by SL Genes

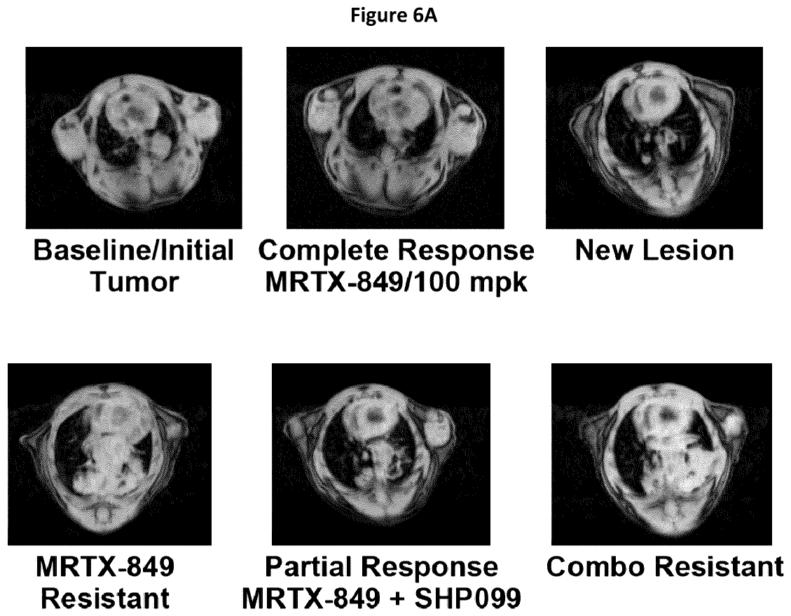

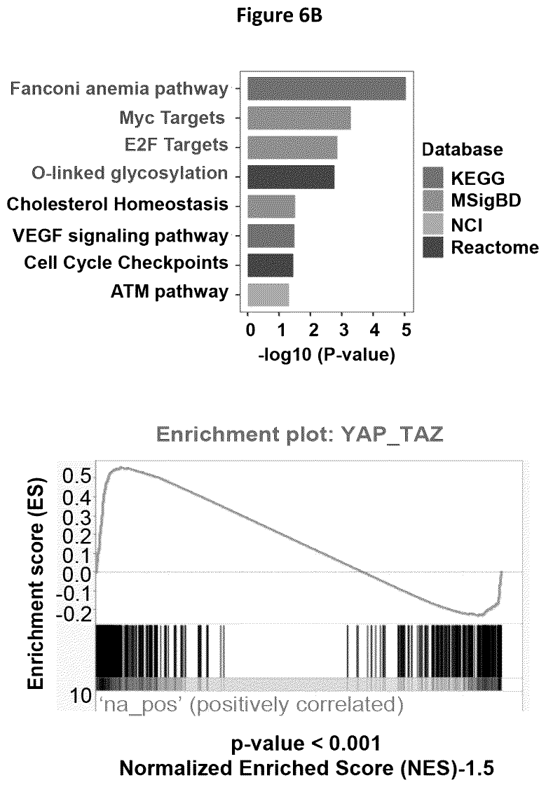

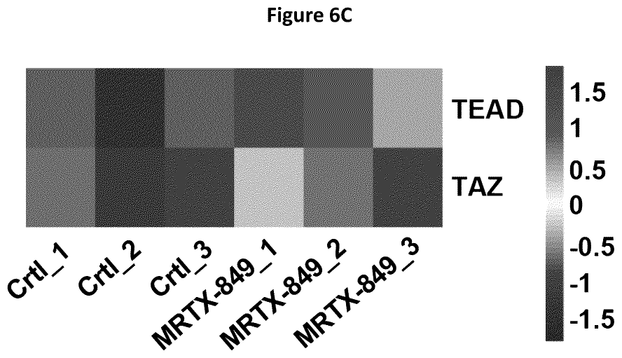

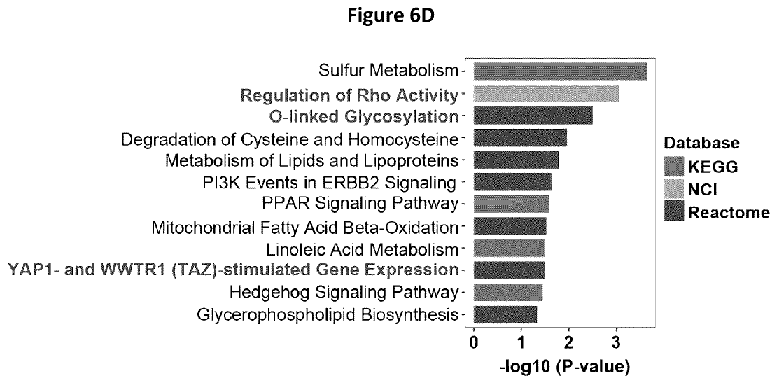

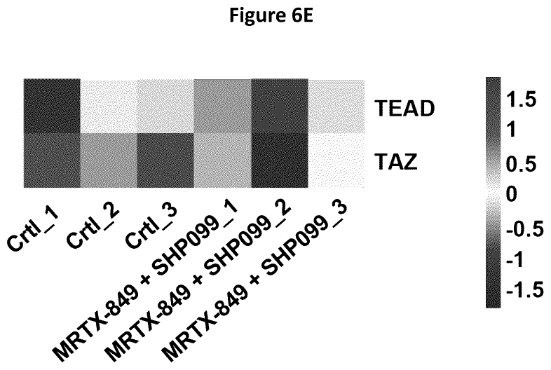

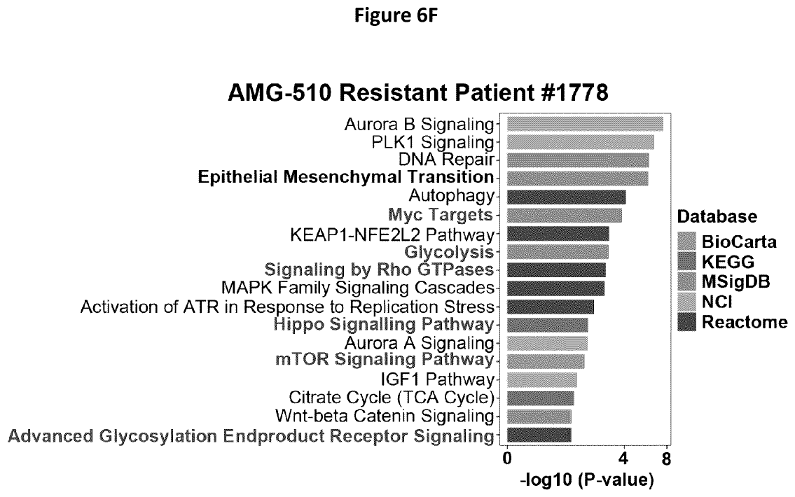

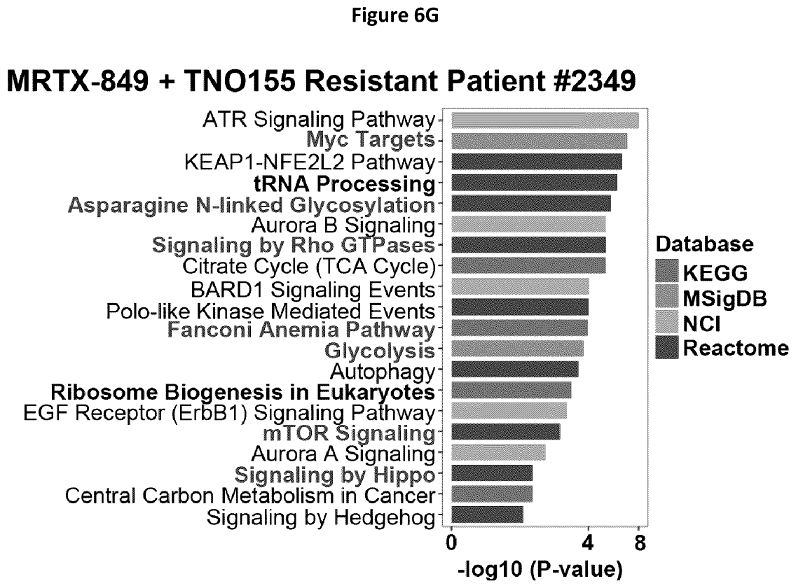

Next, the relevance of the screen findings to the emergence of G12Ci resistance in vivo was further assessed. To this end, G12Ci and G12Ci+SHP2i resistant genetically engineered mouse models, GEMM, were developed (see Methods for details). Briefly, LSL-KRAS^{G12C}; fl/fl Stk11 (KCL) mice were infected with Ad-Cre to simultaneously activate the human KRAS transgene and delete mouse Stk11. Mice were serially monitored by MRI. When tumors reached 300 mm³ (2-3 months), mice were subjected to long-term treatment with 100 mg/kg/daily MRTX-849 (5 days on/2 days off) until tumors recurred (FIG. 6A). Three independent mice were euthanized to obtain nodules. Each nodule was then split in two, and samples were subsequently analyzed by RNA-seq and reverse phase protein array (RPPA). These results were compared with parental tumor samples, obtained from vehicle treated-mice.

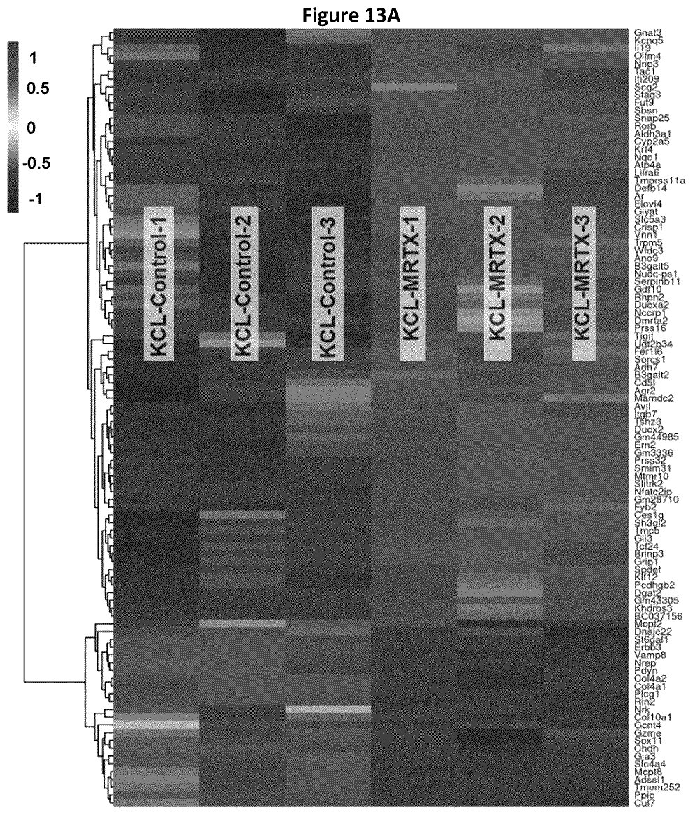

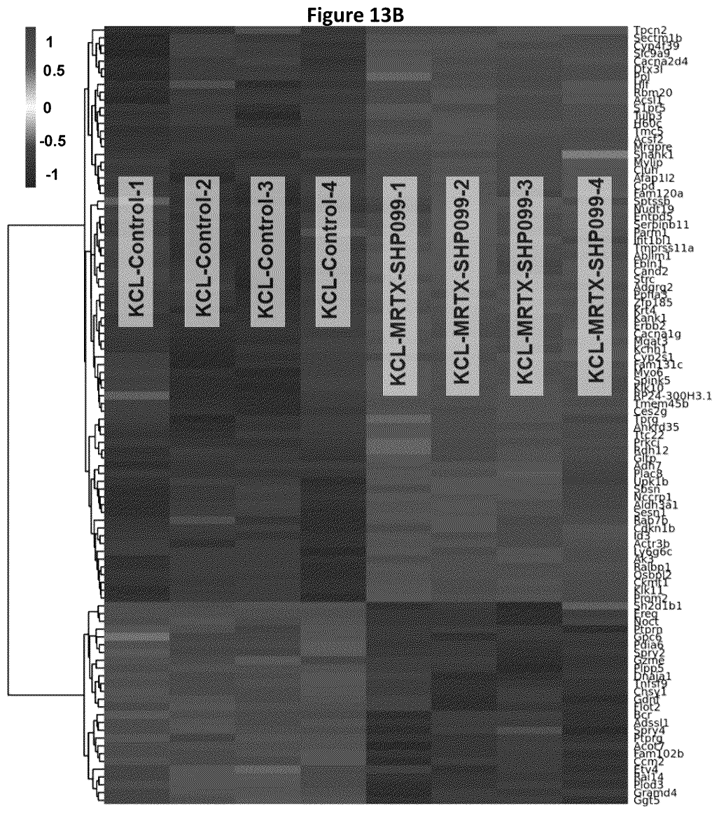

RNAseq analysis revealed multiple enriched pathways (p<0.05) that were also enriched in the CRISPR/Cas9 screens, including Fanconi anemia, MYC, E2F target, and glycosylation genes (FIG. 6B, top panel, starred in red). Importantly, YAP/TAZ pathway signature genes (40) were markedly enriched in resistant nodules (FIG. 6B, bottom panel; FIG. 13A, Table 6). In line with these findings TAZ and TEAD protein were induced in RPPAs from MRTX-849 resistant tumors (FIG. 6C).

TABLE 6
RNA-seq analysis of MTRX-849 (adagrasib)-resistant tumor nodules from KRAS^{G12C}/Stk11−/− mice, showing select
genes, i.e., RHO genes and synthetic lethal genes from MRTX-849 CRISPR/Cas9 screens that overlap in 2 or more lines.

| | KCL- | KCL- | KCL- | | | | | | | | |
|---|---|---|---|---|---|---|---|---|---|---|---|
| | Con- | Con- | Con- | KCL- | KCL- | KCL- | base | | log2FC | | |
| gene | trol-1 | trol-2 | trol-3 | MRTX-1 | MRTX-2 | MRTX-3 | Mean | log2FC | unshrunk | pvalue | padj |
| Aasdhppt | 935.16 | 816.82 | 687.7 | 944.32 | 864.24 | 976.1 | 870.7 | 0.09 | 0.19 | 0.3616 | 0.65911 |
| Actr2 | 23430.13 | 14421.77 | 15613.81 | 18999.34 | 21198.23 | 22199.43 | 19310.5 | 0.09 | 0.22 | 0.41422 | 0.7027 |
| Actr3 | 10499.36 | 8027.24 | 6367.12 | 10850.75 | 9834.06 | 10322.26 | 9316.8 | 0.15 | 0.32 | 0.22248 | 0.51648 |
| Actr6 | 300.62 | 267.95 | 195.17 | 279.53 | 323.22 | 344.51 | 285.2 | 0.14 | 0.31 | 0.24607 | 0.54219 |
| Adat3 | 4.27 | 0 | 0 | 1.79 | 2.77 | 1.85 | 1.8 | 0.03 | 0.54 | 0.76331 | |
| Adnp | 50.39 | 70.46 | 58.25 | 72.57 | 65.2 | 60.2 | 62.8 | 0.05 | 0.15 | 0.62605 | 0.83967 |
| Ahcy | 918.93 | 534.04 | 360.71 | 787.53 | 676.97 | 747.36 | 670.9 | 0.09 | 0.29 | 0.4698 | 0.74298 |
| Ahcyl1 | 3646.68 | 3705.81 | 4467.51 | 4038.01 | 4385.03 | 3913.68 | 4026.1 | 0.03 | 0.06 | 0.73261 | 0.89331 |
| Aifm1 | 1298.97 | 1122.77 | 1102.57 | 1209.52 | 1263.76 | 1254.86 | 1208.7 | 0.04 | 0.08 | 0.62458 | 0.83872 |
| Alg1 | 567.07 | 674.04 | 731.64 | 665.68 | 536.86 | 500.09 | 612.6 | −0.1 | −0.21 | 0.36185 | 0.65932 |
| Alg13 | 264.75 | 177.09 | 204.37 | 353.9 | 355.13 | 401.93 | 292.9 | 0.59 | 0.78 | 0.00187 | 0.02697 |
| Alg2 | 1257.98 | 1756.94 | 1148.56 | 1289.26 | 1165.27 | 1104.83 | 1287.1 | −0.1 | −0.23 | 0.37275 | 0.66918 |
| Alg5 | 1063.26 | 874.3 | 858.35 | 1371.69 | 1108.4 | 1191.88 | 1078 | 0.25 | 0.39 | 0.05593 | 0.2381 |
| Al9 | 1158.91 | 849.27 | 728.58 | 1180.85 | 919.73 | 966.84 | 967.4 | 0.0 | 0.16 | 0.54898 | 0.7945 |
| Ankrd49 | 627.71 | 346.75 | 368.89 | 561.76 | 564.6 | 620.48 | 515 | 0.16 | 0.38 | 0.23265 | 0.52713 |
| Ap2s1 | 2269.99 | 1443.57 | 1416.28 | 2992.44 | 2072.52 | 2534.72 | 2121.6 | 0.29 | 0.57 | 0.06371 | 0.25608 |
| Arf1 | 15538.1 | 11868.41 | 9714.69 | 14386.13 | 11953.74 | 12898.66 | 12726.6 | 0.03 | 0.08 | 0.7573 | 0.90292 |
| Arl2 | 745.56 | 815.89 | 606.98 | 601.18 | 389.81 | 432.49 | 598.7 | −0.36 | −0.61 | 0.02875 | 0.1588 |
| Armc5 | 654.18 | 709.27 | 668.29 | 805.45 | 564.6 | 604.74 | 667.8 | −0.02 | −0.04 | 0.86276 | 0.9482 |
| Armc7 | 254.5 | 190.99 | 189.04 | 221.3 | 198.37 | 192.63 | 207.8 | −0.02 | −0.05 | 0.83039 | 0.93383 |
| Arpc4 | 5533.22 | 3681.7 | 3837.03 | 5097.91 | 4455.78 | 5048.15 | 4609 | 0.07 | 0.16 | 0.51865 | 0.77549 |
| Ascc3 | 3489.54 | 3034.55 | 2734.46 | 2951.23 | 3588.76 | 3517.31 | 3219.3 | 0.05 | 0.12 | 0.55626 | 0.79831 |
| Asna1 | 1566.28 | 1417.61 | 1332.49 | 1585.82 | 1331.74 | 1300.24 | 1422.4 | −0.01 | −0.03 | 0.86085 | 0.94747 |
| Atp6v1b2 | 4360.64 | 5908.71 | 9967.09 | 8833.09 | 11708.2 | 7936.64 | 8119.1 | 0.18 | 0.49 | 0.20325 | 0.49104 |
| Atp6v1d | 2199.96 | 2052.7 | 1973.18 | 2201.33 | 2344.42 | 2291.16 | 2177.1 | 0.07 | 0.13 | 0.35207 | 0.65028 |
| Atpov1e1 | 3088.15 | 3617.73 | 3610.18 | 3362.47 | 3484.72 | 3110.75 | 3379 | −0.02 | −0.05 | 0.76078 | 0.90475 |
| Atp6v1f | 1454.4 | 1768.07 | 1583.86 | 2103.67 | 1444.1 | 1504.91 | 1643.2 | 0.03 | 0.07 | 0.76896 | 0.90826 |
| B3gnt2 | 74.3 | 64.9 | 71.53 | 89.59 | 62.43 | 95.39 | 76.4 | 0.09 | 0.24 | 0.42148 | 0.70833 |
| Bccip | 1621.79 | 1118.14 | 1066.81 | 1764.11 | 1882.47 | 2042.97 | 1582.7 | 0.37 | 0.58 | 0.02335 | 0.13959 |
| Bptf | 5918.38 | 6727.38 | 5895.03 | 4765.51 | 5443.48 | 4939.79 | 5614.9 | −0.19 | −0.29 | 0.07503 | 0.28252 |
| Brk1 | 2217.04 | 2608.06 | 2002.82 | 2641.24 | 2276.44 | 2011.48 | 2292.8 | 0.01 | 0.02 | 0.92352 | 0.97221 |
| Bub3 | 2474.96 | 1623.43 | 1378.47 | 3568.54 | 2952.02 | 3447.85 | 2574.2 | 0.58 | 0.86 | 0.0042 | 0.04617 |
| Ccnc | 1117.92 | 1252.58 | 1085.2 | 1179.96 | 1302.61 | 1298.39 | 1206.1 | 0.06 | 0.13 | 0.43357 | 0.71752 |
| Cct4 | 7547.86 | 5446.06 | 4085.34 | 6772.42 | 6610.14 | 6806.8 | 6211.4 | 0.1 | 0.24 | 0.40842 | 0.69845 |
| Cd3eap | 704.57 | 268.87 | 332.1 | 924.61 | 792.1 | 929.8 | 658.7 | 0.51 | 1.02 | 0.01622 | 0.11118 |
| Cdipt | 2364.79 | 2448.6 | 2032.45 | 3688.59 | 2785.55 | 3045 | 2727.5 | 0.32 | 0.48 | 0.0238 | 0.1412 |
| Cdk7 | 1332.28 | 1070.85 | 870.61 | 974.78 | 1030.71 | 1116.87 | 1066 | −0.03 | −0.07 | 0.77822 | 0.91207 |
| Cenpn | 193.01 | 65.83 | 51.09 | 276.85 | 264.96 | 276.9 | 188.1 | 0.63 | 1.4 | 0.00973 | 0.08058 |
| Cenpo | 297.2 | 241.99 | 253.42 | 403.17 | 369 | 395.44 | 326.7 | 0.43 | 0.56 | 0.00434 | 0.04717 |
| Chchd4 | 545.72 | 589.67 | 483.33 | 620.89 | 545.18 | 551.03 | 556 | 0.04 | 0.09 | 0.65117 | 0.85233 |
| Chmp7 | 1321.17 | 1288.73 | 1355.99 | 1190.71 | 1215.21 | 1031.67 | 1233.9 | −0.11 | −0.21 | 0.20593 | 0.49469 |
| Chtf8 | 324.53 | 196.56 | 145.1 | 197.11 | 166.47 | 241.71 | 211.9 | −0.04 | −0.14 | 0.72416 | 0.88904 |
| Cinp | 801.93 | 506.22 | 397.5 | 909.38 | 903.09 | 880.72 | 733.1 | 0.34 | 0.66 | 0.04446 | 0.2076 |
| Cltc | 32097.61 | 33868.68 | 29871.63 | 31705.58 | 33845.59 | 32130.88 | 32253.3 | 0.01 | 0.03 | 0.84851 | 0.94069 |
| Cnot1 | 10624.05 | 10009.48 | 9563.46 | 9247.91 | 10003.3 | 9600.83 | 9841.5 | −0.03 | −0.07 | 0.6413 | 0.84735 |
| Coasy | 837.8 | 870.59 | 809.3 | 670.16 | 627.03 | 549.17 | 727.3 | −0.34 | −0.45 | 0.01149 | 0.09018 |
| Cops6 | 1831.03 | 1571.51 | 1311.03 | 1884.17 | 1603.64 | 1764.21 | 1660.9 | 0.07 | 0.16 | 0.47439 | 0.74659 |
| Cox11 | 239.98 | 284.63 | 247.29 | 250.86 | 220.57 | 255.6 | 249.8 | −0.04 | −0.08 | 0.68214 | 0.86807 |
| Cox17 | 386.02 | 350.46 | 251.37 | 458.72 | 395.36 | 404.7 | 374.4 | 0.17 | 0.35 | 0.17438 | 0.45412 |
| Cpsf1 | 2884.89 | 2150.98 | 1973.18 | 2065.15 | 1738.2 | 1798.48 | 2101.8 | −0.16 | −0.32 | 0.17931 | 0.46084 |
| Cpsf4 | 697.74 | 440.39 | 456.77 | 672.85 | 535.47 | 631.6 | 572.5 | 0.08 | 0.21 | 0.46878 | 0.74231 |
| Crk | 8406.15 | 6294.4 | 5864.38 | 8183.53 | 8270.65 | 8929.41 | 7658.1 | 0.16 | 0.3 | 0.17657 | 0.45645 |
| Cstf1 | 853.17 | 833.51 | 600.85 | 947.91 | 768.52 | 785.33 | 798.2 | 0.05 | 0.13 | 0.59852 | 0.82323 |
| Ctnnbl1 | 1152.08 | 813.11 | 776.6 | 826.06 | 699.16 | 787.18 | 842.4 | −0.11 | −0.25 | 0.33099 | 0.6301 |
| Cul2 | 1711.46 | 1639.2 | 1432.63 | 1730.06 | 1803.4 | 1749.39 | 1677.7 | 0.07 | 0.14 | 0.3805 | 0.67618 |
| Dap3 | 1248.58 | 1108.87 | 963.6 | 1301.8 | 1047.36 | 1075.2 | 1124.2 | 0.02 | 0.04 | 0.83584 | 0.93597 |
| Dars2 | 503.02 | 363.44 | 329.03 | 559.96 | 525.76 | 474.16 | 459.2 | 0.19 | 0.38 | 0.13942 | 0.40281 |
| Dbr1 | 617.46 | 432.98 | 364.8 | 624.47 | 541.02 | 666.79 | 541.3 | 0.17 | 0.37 | 0.19606 | 0.48329 |
| Dclre1b | 457.76 | 452.45 | 433.26 | 534.88 | 567.38 | 485.27 | 488.5 | 0.14 | 0.24 | 0.1627 | 0.43728 |
| Ddost | 7326.67 | 6706.05 | 5987 | 7860.1 | 6124.61 | 7121.67 | 6854.3 | 0.03 | 0.08 | 0.70303 | 0.87866 |
| Ddx51 | 576.47 | 470.99 | 384.21 | 645.08 | 621.48 | 597.33 | 549.3 | 0.21 | 0.38 | 0.10864 | 0.35003 |
| Ddx59 | 410.79 | 208.61 | 232.98 | 298.35 | 280.22 | 287.09 | 286.3 | 0.01 | 0.02 | 0.94984 | 0.9825 |
| Ddx6 | 16256.34 | 196481 | 16916.66 | 13417.62 | 14495.15 | 13976.63 | 15785.1 | −0.23 | −0.33 | 0.04412 | 0.20703 |
| Dexi | 254.5 | 418.14 | 316.77 | 300.14 | 255.25 | 216.71 | 293.6 | −0.15 | −0.36 | 0.23253 | 0.52699 |
| Dhps | 576.47 | 470.06 | 488.44 | 577.88 | 498.01 | 523.24 | 522.4 | 0.03 | 0.06 | 0.76031 | 0.90462 |
| Dhx33 | 1050.45 | 1036.55 | 871.63 | 1119.03 | 1048.74 | 995.55 | 1020.3 | 0.04 | 0.1 | 0.59311 | 0.82045 |
| Dis3 | 1117.92 | 749.13 | 535.45 | 1539.23 | 1546.76 | 1478.97 | 1161.2 | 0.59 | 0.93 | 0.00512 | 0.05276 |
| Dkc1 | 1703.78 | 818.67 | 681.57 | 2079.48 | 2171.01 | 2188.36 | 1607.1 | 0.53 | 1.01 | 0.01305 | 0.09696 |
| Dnajb11 | 3349.48 | 2837.07 | 2798.84 | 3855.24 | 3744.13 | 3670.12 | 3375.8 | 0.22 | 0.33 | 0.04883 | 0.21929 |
| Dnajc17 | 253.64 | 131.65 | 145.1 | 165.75 | 156.76 | 200.96 | 175.6 | −0.01 | −0.02 | 0.95742 | 0.98547 |
| Dnajc9 | 1012.02 | 817.74 | 579.39 | 1223.86 | 1165.27 | 1094.64 | 982.2 | 0.29 | 0.53 | 0.05738 | 0.2413 |
| Dohh | 171.66 | 203.97 | 118.53 | 216.82 | 131.79 | 169.48 | 168.7 | 0.02 | 0.07 | 0.83746 | 0.93631 |
| Dolk | 542.3 | 521.98 | 566.1 | 546.52 | 478.59 | 480.64 | 522.7 | −0.05 | −0.11 | 0.50769 | 0.76867 |
| Dpagt1 | 651.62 | 898.41 | 775.58 | 954.18 | 740.78 | 701.98 | 787.1 | 0.02 | 0.04 | 0.85933 | 0.94649 |
| Dscc1 | 87.11 | 24.11 | 15.33 | 208.75 | 166.47 | 160.21 | 110.3 | 1.1 | 2.08 | 0.00139 | 0.02184 |
| Dtymk | 959.07 | 656.42 | 450.63 | 1207.73 | 796.27 | 993.7 | 844 | 0.22 | 0.54 | 0.14355 | 0.40897 |
| Dyrk1a | 3319.59 | 2990.98 | 3019.56 | 2815.05 | 2899.31 | 2706.05 | 2958.4 | −0.08 | −0.15 | 0.30756 | 0.60913 |
| Ears2 | 284.39 | 229.01 | 166.56 | 398.69 | 338.48 | 344.51 | 293.6 | 0.41 | 0.67 | 0.01958 | 0.12479 |
| Eif1ad | 1064.97 | 672.18 | 680.55 | 1379.75 | 1216.6 | 1254.86 | 1044.8 | 0.43 | 0.67 | 0.0148 | 0.10501 |
| Eif2b5 | 2423.72 | 1683.7 | 1454.09 | 2143.99 | 1804.78 | 1943.87 | 1909 | 0.03 | 0.08 | 0.76317 | 0.90591 |
| Eif3a | 18457.15 | 10577.82 | 11147.32 | 11590.8 | 14524.28 | 15185.19 | 13580.4 | 0.01 | 0.04 | 0.9 | 0.96265 |
| Eif3f | 5850.06 | 6012.55 | 5413.74 | 7605.65 | 4941.31 | 5219.47 | 5840.5 | 0.02 | 0.04 | 0.87321 | 0.95293 |
| Eif3h | 5985.85 | 4920.37 | 4060.82 | 5266.34 | 4077.06 | 4518.42 | 4804.8 | −0.05 | −0.11 | 0.65335 | 0.85368 |
| Elp3 | 1638.02 | 1577.08 | 1283.44 | 1377.96 | 1369.19 | 1226.15 | 1412 | −0.09 | −0.18 | 0.35529 | 0.65321 |
| Elp5 | 738.73 | 667.55 | 655 | 723.02 | 683.9 | 662.16 | 688.4 | 0 | 0.01 | 0.97424 | 0.99144 |
| Emc1 | 3823.46 | 4551.36 | 5115.36 | 3962.75 | 4439.13 | 3449.71 | 4223.6 | −0.08 | −0.19 | 0.39761 | 0.69054 |
| Emc6 | 930.03 | 1916.41 | 1321.25 | 1150.39 | 826.79 | 848.3 | 1165.5 | −0.24 | −0.56 | 0.11274 | 0.35817 |
| Eno1 | 13258.71 | 4065.54 | 4732.17 | 10143.85 | 6557.43 | 8744.19 | 7917 | 0.05 | 0.21 | 0.68985 | 0.87183 |
| Erbb3 | 7428.3 | 7571.08 | 7974.49 | 3835.53 | 3930.02 | 3672.89 | 5735.4 | −0.96 | −1.01 | 7.443E−14 | 2.2952E−11 |
| Ercc1 | 670.41 | 429.27 | 312.68 | 850.25 | 595.12 | 822.37 | 613.4 | 0.31 | 0.68 | 0.06414 | 0.25702 |
| Ercc2 | 848.9 | 650.86 | 594.71 | 885.19 | 667.26 | 810.33 | 742.9 | 0.07 | 0.17 | 0.49007 | 0.75687 |
| Exoc2 | 2089.8 | 1854.29 | 1885.31 | 2065.15 | 2386.03 | 2068.9 | 2058.2 | 0.08 | 0.16 | 0.33965 | 0.63839 |
| Exosc10 | 1411.7 | 1168.21 | 977.91 | 1522.2 | 1439.94 | 1379.88 | 1316.6 | 0.15 | 0.29 | 0.19599 | 0.48329 |
| Exosc2 | 653.33 | 379.2 | 381.15 | 1022.27 | 833.72 | 854.79 | 687.4 | 0.64 | 0.94 | 0.0029 | 0.03597 |
| Exosc4 | 548.28 | 511.79 | 418.96 | 572.51 | 345.42 | 484.35 | 480.2 | −0.03 | −0.08 | 0.79316 | 0.91735 |
| Ext1 | 4123.22 | 2445.81 | 2463.67 | 2781 | 2806.36 | 2862.56 | 2913.8 | −0.04 | −0.1 | 0.74193 | 0.89658 |
| Ext2 | 2080.4 | 2002.64 | 1799.47 | 2255.98 | 1691.03 | 1754.95 | 1930.7 | −0.02 | −0.04 | 0.83316 | 0.93505 |
| Fars2 | 609.77 | 560 | 697.92 | 699.73 | 701.94 | 614 | 647.2 | 0.05 | 0.11 | 0.57031 | 0.80677 |
| Fbl | 2126.52 | 499.73 | 604.93 | 1271.34 | 1541.21 | 2009.63 | 1342.2 | 0.14 | 0.58 | 0.33281 | 0.63152 |
| Fdxr | 155.43 | 179.87 | 146.12 | 111.1 | 74.91 | 110.21 | 129.6 | −0.44 | −0.69 | 0.01339 | 0.09832 |
| Fkbpl | 178.49 | 143.71 | 139.99 | 119.16 | 104.04 | 126.88 | 135.4 | −0.21 | −0.4 | 0.10824 | 0.34943 |
| Fntb | 493.63 | 407.94 | 300.42 | 529.5 | 496.63 | 461.2 | 448.2 | 0.14 | 0.31 | 0.25431 | 0.55175 |
| Fxn | 164.83 | 175.23 | 136.93 | 171.12 | 113.75 | 107.43 | 144.9 | −0.11 | −0.28 | 0.36132 | 0.65887 |
| Gapdh | 27666.09 | 13011.58 | 15878.47 | 20266.2 | 17985.41 | 22327.23 | 19522.5 | 0.03 | 0.1 | 0.77987 | 0.91274 |
| Gemin7 | 725.07 | 720.39 | 511.94 | 948.8 | 719.97 | 833.49 | 743.3 | 0.17 | 0.35 | 0.16298 | 0.43758 |
| Gfm1 | 1312.63 | 1130.19 | 1053.52 | 1571.48 | 1725.71 | 1565.1 | 1393.1 | 0.36 | 0.48 | 0.00919 | 0.07736 |
| Gfpt1 | 15620.09 | 12071.45 | 8015.36 | 18299.61 | 15835.21 | 18244.08 | 14681 | 0.27 | 0.55 | 0.07626 | 0.28489 |
| Ggnbp2 | 2902.83 | 2672.96 | 2589.36 | 2970.05 | 2879.89 | 3036.67 | 2842 | 0.06 | 0.12 | 0.39634 | 0.68986 |
| Ggps1 | 1503.94 | 1795.88 | 1524.59 | 1537.44 | 1628.61 | 1518.8 | 1584.9 | −0.02 | −0.04 | 0.80225 | 0.92171 |
| Gmppb | 1363.02 | 795.49 | 686.68 | 1868.93 | 1457.98 | 1736.43 | 1318.1 | 0.48 | 0.83 | 0.01535 | 0.10733 |
| Gmps | 3448.55 | 2853.76 | 2219.45 | 3721.74 | 3692.8 | 3486.75 | 3237.2 | 0.18 | 0.36 | 0.13901 | 0.40207 |
| Gnb1l | 106.75 | 101.99 | 60.29 | 156.79 | 112.37 | 132.43 | 111.8 | 0.27 | 0.58 | 0.09087 | 0.31665 |
| Gpn2 | 293.78 | 272.58 | 228.89 | 361.06 | 224.73 | 346.36 | 287.9 | 0.09 | 0.23 | 0.42225 | 0.70899 |
| Grb2 | 1846.4 | 2318.79 | 2955.18 | 3042.62 | 3340.45 | 2405.07 | 2651.4 | 0.13 | 0.3 | 0.28017 | 0.57981 |
| Grpel1 | 1597.02 | 1520.52 | 1366.21 | 1988.09 | 1542.6 | 1736.43 | 1625.1 | 0.12 | 0.23 | 0.23582 | 0.53011 |
| Grwd1 | 807.91 | 369 | 272.83 | 1478.3 | 1077.88 | 1240.04 | 874.3 | 0.81 | 1.39 | 0.00254 | 0.03299 |
| Gtf2h3 | 883.91 | 450.59 | 371.95 | 653.14 | 617.32 | 621.41 | 599.7 | 0.05 | 0.15 | 0.70429 | 0.8791 |
| Gtf3c1 | 6191.67 | 5684.34 | 5009.09 | 5878.27 | 5551.69 | 5971.46 | 5714.4 | 0.02 | 0.04 | 0.79856 | 0.9197 |
| Guk1 | 645.64 | 936.42 | 708.14 | 863.69 | 595.12 | 611.22 | 726.7 | −0.06 | −0.14 | 0.61103 | 0.83171 |
| Hdac3 | 2919.91 | 1775.49 | 1429.56 | 2245.23 | 1860.27 | 2080.94 | 2051.9 | 0 | 0.01 | 0.96616 | 0.98829 |
| Hnf1b | 427.01 | 943.84 | 777.62 | 702.42 | 549.34 | 530.65 | 655.1 | −0.09 | −0.27 | 0.46487 | 0.7396 |
| Hnrnpu | 19710.86 | 14762.03 | 12889.57 | 18435.79 | 19665.34 | 21735.46 | 17866.5 | 0.17 | 0.34 | 0.17126 | 0.44942 |
| Hsd17b10 | 1579.94 | 2440.25 | 2092.74 | 1433.51 | 1168.05 | 1220.59 | 1655.8 | −0.47 | −0.68 | 0.0079 | 0.07021 |
| Huwe1 | 18958.47 | 21363.32 | 21128.71 | 18141.03 | 20020.47 | 18656.19 | 197114 | −0.06 | −0.11 | 0.45298 | 0.73197 |
| Hyou1 | 8576.1 | 8106.05 | 7635.23 | 10480.72 | 9807.7 | 9577.68 | 9030.6 | 0.21 | 0.3 | 0.04293 | 0.20392 |
| Iars | 2560.36 | 2313.23 | 2021.21 | 2844.61 | 2872.95 | 2658.82 | 2545.2 | 0.16 | 0.28 | 0.11858 | 0.36714 |
| Iba57 | 422.74 | 330.06 | 321.88 | 270.57 | 266.35 | 308.39 | 320 | −0.19 | −0.35 | 0.12601 | 0.38002 |
| Il6st | 6855.25 | 11920.33 | 15088.58 | 9459.35 | 13021.91 | 7838.47 | 10697.3 | −0.05 | −0.16 | 0.68088 | 0.86746 |
| Ilf2 | 2526.2 | 1653.1 | 1329.42 | 2453.09 | 2128.01 | 2399.51 | 2081.6 | 0.14 | 0.34 | 0.27359 | 0.57262 |
| Ilf3 | 2379.31 | 1736.55 | 1512.33 | 1840.26 | 1641.09 | 1679.01 | 1798.1 | −0.05 | −0.13 | 0.62184 | 0.83773 |
| Imp3 | 790.83 | 1065.29 | 774.56 | 851.14 | 584.02 | 670.49 | 789.4 | −0.15 | −0.32 | 0.23492 | 0.52919 |
| Ints10 | 895.87 | 518.28 | 541.58 | 853.83 | 930.83 | 864.05 | 767.4 | 0.2 | 0.44 | 0.14582 | 0.41211 |
| Ipo11 | 1278.47 | 1241.45 | 892.07 | 1457.7 | 1290.12 | 1388.22 | 1258 | 0.13 | 0.28 | 0.23006 | 0.52384 |
| Ipo9 | 3434.03 | 2899.19 | 2593.45 | 3080.25 | 2958.96 | 2912.57 | 2979.7 | 0 | 0 | 0.98359 | 0.99444 |
| Jmjd6 | 942.84 | 642.51 | 414.87 | 1188.91 | 954.41 | 1129.84 | 878.9 | 0.34 | 0.71 | 0.05001 | 0.22186 |
| Kdm2a | 5836.4 | 4641.3 | 4031.18 | 4785.22 | 4258.79 | 4667.52 | 4703.4 | −0.03 | −0.08 | 0.71595 | 0.8849 |
| Krr1 | 1463.8 | 1166.35 | 1161.84 | 1437.09 | 1387.23 | 1528.06 | 1357.4 | 0.1 | 0.2 | 0.30254 | 0.60378 |
| Kti12 | 571.34 | 423.71 | 286.12 | 619.99 | 499.4 | 536.21 | 489.5 | 0.15 | 0.37 | 0.26131 | 0.55872 |
| Lemd2 | 1726.84 | 1027.28 | 1080.09 | 1297.32 | 1057.07 | 1039.08 | 1204.6 | −0.07 | −0.18 | 0.55877 | 0.79965 |
| Lin52 | 202.4 | 257.75 | 218.68 | 220.4 | 266.35 | 208.37 | 229 | 0.01 | 0.03 | 0.89537 | 0.96182 |
| Lsm10 | 376.62 | 416.29 | 360.71 | 304.62 | 273.28 | 211.15 | 323.8 | −0.36 | −0.55 | 0.02196 | 0.13428 |
| Mad2l2 | 319.4 | 505.3 | 442.46 | 311.79 | 233.05 | 248.19 | 343.4 | −0.41 | −0.67 | 0.01867 | 0.12114 |
| Mars2 | 730.19 | 722.25 | 667.27 | 947.91 | 860.08 | 897.39 | 804.2 | 0.26 | 0.35 | 0.02451 | 0.1433 |
| Mbtps2 | 1068.38 | 1108.87 | 1200.67 | 1375.27 | 1370.58 | 1337.28 | 1243.5 | 0.18 | 0.27 | 0.06457 | 0.25814 |
| Mcl1 | 12169.83 | 120798 | 10841.79 | 15333.14 | 13915.28 | 13697.88 | 13006.3 | 0.19 | 0.29 | 0.05932 | 0.24542 |
| Mcmbp | 3240.16 | 2419.85 | 1994.64 | 4317.54 | 3835.69 | 4078.52 | 3314.4 | 0.46 | 0.68 | 0.00897 | 0.07619 |
| Mettl3 | 668.7 | 491.39 | 395.45 | 482.91 | 524.37 | 551.03 | 519 | 0 | 0 | 0.99503 | 0.99801 |
| Mipep | 523.52 | 509.93 | 356.62 | 721.23 | 578.47 | 580.66 | 545.1 | 0.23 | 0.44 | 0.09412 | 0.32284 |
| Mis18a | 479.11 | 238.28 | 197.22 | 565.34 | 474.43 | 530.65 | 414.2 | 0.35 | 0.78 | 0.05362 | 0.23191 |
| Mms19 | 1273.35 | 1068.07 | 1147.53 | 1127.99 | 1054.29 | 985.37 | 1109.4 | −0.07 | −0.14 | 0.42742 | 0.71244 |
| Mob4 | 3445.13 | 2527.4 | 1972.16 | 3274.67 | 3361.25 | 3780.32 | 3060.2 | 0.18 | 0.39 | 0.16611 | 0.44235 |
| Mocs3 | 186.18 | 177.09 | 121.6 | 216.82 | 134.56 | 148.18 | 164.1 | 0.02 | 0.05 | 0.89181 | 0.96043 |
| Mogs | 1647.41 | 1553.9 | 1057.61 | 1799.05 | 1376.13 | 1363.21 | 1466.2 | 0.03 | 0.09 | 0.74283 | 0.8971 |
| Mpdu1 | 1807.97 | 1405.55 | 1493.94 | 1539.23 | 1467.69 | 1449.34 | 1527.3 | −0.04 | −0.08 | 0.67454 | 0.86482 |
| Mpi | 477.4 | 915.09 | 739.82 | 462.31 | 405.07 | 406.56 | 567.7 | −0.44 | −0.74 | 0.01826 | 0.11959 |
| Mrpl21 | 655.89 | 484.9 | 404.65 | 627.16 | 446.69 | 532.5 | 525.3 | 0.02 | 0.06 | 0.84738 | 0.94026 |
| Mrpl28 | 940.28 | 882.64 | 672.37 | 1072.44 | 767.14 | 827.93 | 860.5 | 0.04 | 0.1 | 0.71045 | 0.88207 |
| Mrpl4 | 566.22 | 737.08 | 577.34 | 696.15 | 567.38 | 449.16 | 598.9 | −0.05 | −0.13 | 0.61974 | 0.83674 |
| Mrpl47 | 412.49 | 463.57 | 404.65 | 446.18 | 434.2 | 437.12 | 433 | 0.02 | 0.04 | 0.81006 | 0.92538 |
| Mrp153 | 2.56 | 0.93 | 0 | 1.79 | 0 | 0.93 | 1 | −0.02 | −0.31 | 0.87586 | |
| Mrps18a | 676.39 | 680.53 | 629.46 | 776.78 | 590.96 | 594.55 | 658.1 | −0.01 | −0.02 | 0.93721 | 0.97783 |
| Mrps24 | 485.09 | 726.88 | 396.48 | 882.5 | 335.71 | 578.81 | 567.6 | 0.05 | 0.16 | 0.71922 | 0.88617 |
| Mrps34 | 622.58 | 557.22 | 521.14 | 710.48 | 438.36 | 555.66 | 567.6 | 0 | 0 | 0.9862 | 0.99504 |
| Mrps6 | 284.39 | 307.81 | 203.35 | 899.53 | 486.92 | 591.77 | 462.3 | 1 | 1.31 | 0.00014 | 0.00398 |
| Mtg1 | 210.09 | 185.43 | 199.26 | 288.49 | 313.51 | 258.38 | 242.5 | 0.39 | 0.53 | 0.00905 | 0.07659 |
| Mtg2 | 342.46 | 313.38 | 276.92 | 310 | 287.16 | 301.91 | 305.3 | −0.02 | −0.05 | 0.78646 | 0.915 |
| Mtor | 2934.42 | 3200.51 | 3163.64 | 2481.76 | 2528.92 | 2276.34 | 2764.3 | −0.27 | −0.35 | 0.01783 | 0.11769 |
| Mtx1 | 661.01 | 481.19 | 381.15 | 718.55 | 542.41 | 611.22 | 565.9 | 0.12 | 0.3 | 0.32096 | 0.62216 |
| Mvd | 637.1 | 573.9 | 475.16 | 823.37 | 638.12 | 749.21 | 649.5 | 0.22 | 0.39 | 0.08743 | 0.30996 |
| N6amt1 | 531.2 | 330.99 | 369.91 | 485.6 | 565.99 | 489.9 | 462.3 | 0.14 | 0.32 | 0.24088 | 0.53552 |
| Naa10 | 491.92 | 426.49 | 315.75 | 642.39 | 362.07 | 457.49 | 449.4 | 0.09 | 0.25 | 0.46151 | 0.73761 |
| Naa20 | 763.5 | 563.71 | 579.39 | 846.66 | 864.24 | 772.36 | 731.6 | 0.22 | 0.38 | 0.08297 | 0.29993 |
| Naa25 | 2916.49 | 1715.22 | 1483.72 | 2687.82 | 2814.69 | 2631.04 | 2374.8 | 0.17 | 0.41 | 0.20924 | 0.49987 |
| Nae1 | 1512.48 | 1116.28 | 1015.72 | 1275.82 | 1138.91 | 1429.89 | 1248.2 | 0.03 | 0.08 | 0.76008 | 0.90462 |
| Nars | 6114.81 | 3712.3 | 4187.53 | 6638.93 | 6328.53 | 6695.67 | 5613 | 0.27 | 0.49 | 0.06712 | 0.26471 |
| Ncapd2 | 1741.35 | 650.86 | 527.27 | 2051.71 | 1685.48 | 1816.07 | 1412.1 | 0.34 | 0.93 | 0.06302 | 0.2547 |
| Ndst1 | 6697.25 | 10737.29 | 13403.56 | 7862.78 | 7595.07 | 6395.61 | 8781.9 | −0.22 | −0.5 | 0.12645 | 0.38058 |
| Ndufs2 | 4790.22 | 3581.57 | 3697.04 | 3782.67 | 3448.65 | 3634.92 | 3822.5 | −0.07 | −0.15 | 0.46209 | 0.73788 |
| Nelfa | 586.71 | 532.18 | 441.44 | 680.02 | 656.16 | 635.3 | 588.6 | 0.2 | 0.34 | 0.09477 | 0.324 |
| Nelfb | 2161.53 | 2036.01 | 1525.62 | 1745.29 | 1310.93 | 1440.08 | 1703.2 | −0.17 | −0.35 | 0.1626 | 0.43728 |
| Nelfcd | 935.16 | 1091.25 | 1041.26 | 835.02 | 710.26 | 689.01 | 883.7 | −0.33 | −0.46 | 0.01553 | 0.10829 |
| Nhlrc2 | 2100.04 | 2257.6 | 1732.03 | 2167.28 | 2012.87 | 1971.66 | 2040.2 | 0.01 | 0.01 | 0.93953 | 0.97867 |
| Nhp2 | 1079.49 | 711.12 | 589.61 | 1480.99 | 1141.69 | 1318.76 | 1053.6 | 0.42 | 0.73 | 0.02041 | 0.12809 |
| Noc4l | 584.15 | 445.03 | 309.62 | 873.54 | 796.27 | 720.5 | 621.5 | 0.54 | 0.84 | 0.00685 | 0.06432 |
| Nol9 | 1186.24 | 939.2 | 649.89 | 1455.01 | 1129.2 | 1136.32 | 1082.6 | 0.19 | 0.42 | 0.17223 | 0.45082 |
| Nop56 | 2369.91 | 1024.5 | 716.31 | 3159.99 | 2458.17 | 2783.84 | 2085.5 | 0.43 | 1.03 | 0.03283 | 0.17273 |
| Nsmce1 | 739.58 | 834.43 | 675.44 | 1182.64 | 848.98 | 1034.45 | 885.9 | 0.27 | 0.45 | 0.04957 | 0.22084 |
| Nsmce2 | 696.88 | 571.12 | 578.36 | 652.25 | 588.18 | 610.3 | 616.2 | 0 | 0 | 0.98467 | 0.99482 |
| Nudc | 1981.33 | 1733.76 | 1221.11 | 1942.4 | 1548.15 | 1929.06 | 1726 | 0.05 | 0.13 | 0.62291 | 0.83822 |
| Ogfr | 1634.6 | 1248.87 | 1257.89 | 1163.83 | 1098.68 | 1060.38 | 1244 | −0.18 | −0.32 | 0.1227 | 0.37488 |
| Oip5 | 137.5 | 40.79 | 32.7 | 199.79 | 147.05 | 167.62 | 120.9 | 0.45 | 1.28 | 0.03076 | 0.16574 |
| Otud5 | 1924.97 | 1741.18 | 1698.31 | 1770.38 | 1405.26 | 1584.55 | 1687.4 | −0.08 | −0.17 | 0.35513 | 0.65301 |
| Paics | 7250.66 | 7595.19 | 6064.66 | 6201.71 | 5598.85 | 5279.67 | 6331.8 | −0.17 | −0.29 | 0.12317 | 0.37536 |
| Pak1ip1 | 1523.58 | 1022.64 | 1000.39 | 1548.19 | 1378.9 | 1453.04 | 1321.1 | 0.14 | 0.3 | 0.23839 | 0.53265 |
| Pak2 | 7937.29 | 6478.9 | 5857.22 | 8496.21 | 7731.02 | 7922.75 | 7403.9 | 0.13 | 0.25 | 0.21153 | 0.50251 |
| Pars2 | 179.34 | 127.95 | 118.53 | 190.84 | 126.24 | 125.02 | 144.7 | 0.02 | 0.06 | 0.8667 | 0.94948 |
| Pdap1 | 3462.21 | 1697.61 | 1671.74 | 2273 | 2423.49 | 2824.59 | 2392.1 | 0.05 | 0.14 | 0.71051 | 0.88207 |
| Pdcd5 | 473.98 | 618.41 | 367.86 | 932.68 | 546.57 | 846.45 | 631 | 0.34 | 0.67 | 0.04747 | 0.21576 |
| Pdcd6ip | 7941.56 | 7817.7 | 6807.54 | 8558.03 | 8233.2 | 8378.39 | 7956.1 | 0.08 | 0.16 | 0.30486 | 0.60641 |
| Pdss1 | 157.14 | 97.35 | 81.75 | 299.24 | 259.41 | 321.36 | 202.7 | 1.1 | 1.39 | 0.00004 | 0.00144 |
| Pdss2 | 233.15 | 237.35 | 185.98 | 247.28 | 266.35 | 194.48 | 227.4 | 0.04 | 0.11 | 0.67387 | 0.8646 |
| Pgd | 6286.47 | 6308.31 | 6005.39 | 8080.5 | 6833.49 | 7076.3 | 6765.1 | 0.14 | 0.24 | 0.12584 | 0.37986 |
| Pgm3 | 899.29 | 1213.64 | 987.1 | 997.18 | 1015.45 | 1046.49 | 1026.5 | −0.01 | −0.02 | 0.92523 | 0.97287 |
| Phf12 | 1350.21 | 1391.65 | 1141.4 | 1385.13 | 1120.88 | 1152.06 | 1256.9 | −0.04 | −0.09 | 0.67101 | 0.8629 |
| Pkn2 | 4800.47 | 4019.18 | 3128.89 | 4091.77 | 3956.37 | 4218.36 | 4035.8 | 0.02 | 0.04 | 0.8725 | 0.95257 |
| Pmpca | 2595.38 | 2114.82 | 1809.69 | 2679.76 | 2426.26 | 2499.53 | 2354.2 | 0.1 | 0.22 | 0.31123 | 0.61316 |
| Pmpcb | 1327.15 | 1271.12 | 1078.05 | 1338.54 | 1197.18 | 1246.52 | 1243.1 | 0.02 | 0.04 | 0.82054 | 0.9297 |
| Polr1e | 268.16 | 187.28 | 112.4 | 329.71 | 266.35 | 260.23 | 237.4 | 0.24 | 0.59 | 0.12468 | 0.378 |
| Polr3k | 1339.96 | 1081.05 | 832.8 | 1317.93 | 1276.25 | 1356.73 | 1200.8 | 0.13 | 0.28 | 0.2675 | 0.56653 |
| Polrmt | 552.55 | 513.64 | 460.85 | 479.33 | 380.1 | 357.47 | 457.3 | −0.17 | −0.33 | 0.14544 | 0.41145 |
| Pop5 | 373.21 | 541.45 | 226.85 | 603.86 | 355.13 | 461.2 | 427 | 0.1 | 0.32 | 0.44447 | 0.72518 |
| Ppp4c | 3277.74 | 2024.89 | 1892.46 | 4646.35 | 3128.2 | 4050.74 | 3170.1 | 0.39 | 0.72 | 0.02842 | 0.15744 |
| Ppp6c | 1839.57 | 2405.02 | 2162.23 | 1901.19 | 1951.83 | 1773.47 | 2005.6 | −0.09 | −0.19 | 0.32805 | 0.62801 |
| Prmt1 | 2640.64 | 1425.02 | 1250.74 | 3479.84 | 2897.92 | 3602.51 | 2549.4 | 0.52 | 0.91 | 0.01227 | 0.09338 |
| Psmg1 | 611.48 | 366.22 | 267.72 | 464.1 | 384.26 | 458.42 | 425.4 | 0.02 | 0.07 | 0.85297 | 0.94264 |
| Psmg4 | 198.13 | 182.65 | 141.01 | 238.32 | 127.62 | 136.14 | 170.6 | −0.02 | −0.05 | 0.88386 | 0.95704 |
| Ptbp1 | 7182.34 | 5262.49 | 4446.05 | 9376.03 | 7547.91 | 7823.65 | 6939.7 | 0.32 | 0.55 | 0.04144 | 0.20052 |
| Ptcd3 | 1445.86 | 916.02 | 873.68 | 1368.1 | 1523.18 | 1583.62 | 1285.1 | 0.23 | 0.47 | 0.10222 | 0.33825 |
| Ptdss1 | 2296.47 | 2066.61 | 1927.2 | 2221.04 | 1954.6 | 1895.72 | 2060.3 | −0.02 | −0.05 | 0.77516 | 0.91102 |
| Ptpmt1 | 675.53 | 561.85 | 588.58 | 829.64 | 618.7 | 625.11 | 649.9 | 0.08 | 0.18 | 0.42167 | 0.70833 |
| Rab10 | 8525.72 | 7041.68 | 5524.1 | 8267.75 | 7552.07 | 7784.76 | 7449.3 | 0.07 | 0.16 | 0.49601 | 0.76078 |
| Rabggtb | 1548.34 | 969.8 | 863.46 | 1142.33 | 1162.5 | 1176.14 | 1143.8 | 0.02 | 0.04 | 0.89002 | 0.95972 |
| Rabif | 1189.65 | 969.8 | 891.05 | 1171.89 | 1097.3 | 1035.37 | 1059.2 | 0.05 | 0.12 | 0.57665 | 0.81052 |
| Rad51d | 452.63 | 377.35 | 389.32 | 387.05 | 346.81 | 331.54 | 380.8 | −0.09 | −0.19 | 0.32509 | 0.62533 |
| Rcl1 | 446.65 | 393.11 | 370.93 | 537.56 | 498.01 | 471.38 | 452.9 | 0.19 | 0.32 | 0.09477 | 0.324 |
| Rho | 1.71 | 1.85 | 2.04 | 0 | 0 | 1.85 | 1.2 | −0.09 | −1.5 | 0.41503 | |
| Rhoa | 12180.08 | 10424.84 | 10370.72 | 10876.73 | 11160.25 | 11316.89 | 11054.9 | 0.01 | 0.02 | 0.91727 | 0.97028 |
| Rhob | 2975.42 | 3274.68 | 2823.36 | 2669.01 | 2386.03 | 2368.03 | 2749.4 | −0.18 | −0.29 | 0.0791 | 0.29097 |
| Rhoc | 1197.34 | 1078.27 | 1203.73 | 1152.18 | 1082.04 | 993.7 | 1117.9 | −0.05 | −0.11 | 0.51718 | 0.77498 |
| Rhod | 712.26 | 572.05 | 385.24 | 668.37 | 450.85 | 643.64 | 572.1 | 0.03 | 0.08 | 0.81374 | 0.92697 |
| Rhof | 445.8 | 233.64 | 247.29 | 569.82 | 536.86 | 556.58 | 431.7 | 0.5 | 0.84 | 0.01181 | 0.09134 |
| Rhog | 1112.79 | 1135.76 | 1121.99 | 1826.82 | 1335.9 | 1450.27 | 1330.6 | 0.3 | 0.45 | 0.02805 | 0.15622 |
| Rhoh | 149.45 | 60.26 | 160.43 | 139.77 | 104.04 | 140.77 | 125.8 | 0.02 | 0.06 | 0.89713 | 0.96204 |
| Rhoj | 575.61 | 455.23 | 509.9 | 447.07 | 332.93 | 369.51 | 448.4 | −0.25 | −0.42 | 0.0674 | 0.26548 |
| Rhoq | 1280.18 | 2734.16 | 3074.74 | 1979.13 | 2284.76 | 1959.62 | 2218.8 | −0.06 | −0.19 | 0.62381 | 0.83859 |
| Rhou | 316.84 | 932.71 | 1146.51 | 988.22 | 948.86 | 927.95 | 876.8 | 0.07 | 0.26 | 0.59485 | 0.82168 |
| Rhov | 579.88 | 12.98 | 23.5 | 464.99 | 274.67 | 462.12 | 303 | 0.11 | 0.96 | | |
| Ric8a | 2024.89 | 1610.45 | 1600.21 | 2419.94 | 1803.4 | 2219.85 | 1946.5 | 0.15 | 0.3 | 0.18903 | 0.47456 |
| Riok2 | 997.5 | 768.6 | 627.41 | 1016.89 | 919.73 | 1064.08 | 899 | 0.15 | 0.33 | 0.20469 | 0.49306 |
| Rnaseh2a | 674.68 | 386.62 | 417.94 | 800.97 | 633.96 | 647.34 | 593.6 | 0.23 | 0.49 | 0.11591 | 0.36302 |
| Rnmt | 1158.06 | 827.94 | 785.8 | 925.51 | 908.63 | 939.99 | 924.3 | 0 | 0 | 0.997 | 0.99896 |
| Rock1 | 6746.78 | 7618.37 | 7844.71 | 6557.4 | 8055.63 | 7674.55 | 7416.2 | 0 | 0 | 0.97834 | 0.9931 |
| Rock2 | 8742.64 | 7987.37 | 5992.11 | 8131.57 | 8584.17 | 8432.1 | 7978.3 | 0.06 | 0.15 | 0.51123 | 0.77085 |
| Rpe | 2024.04 | 1704.1 | 1479.63 | 2052.6 | 1974.03 | 2249.49 | 1914 | 0.14 | 0.27 | 0.19769 | 0.48486 |
| Rpl14 | 9872.51 | 7752.8 | 5210.39 | 9824.9 | 5369.96 | 6113.16 | 7357.3 | −0.03 | −0.1 | 0.79203 | 0.91696 |
| Rpn1 | 7656.32 | 6953.6 | 6403.91 | 10473.56 | 8700.69 | 9201.68 | 8231.6 | 0.32 | 0.43 | 0.01682 | 0.11378 |
| Rpp21 | 578.17 | 320.79 | 259.55 | 508.89 | 335.71 | 406.56 | 401.6 | 0.03 | 0.11 | 0.77882 | 0.9123 |
| Rpp38 | 182.76 | 109.4 | 69.49 | 177.4 | 184.5 | 139.84 | 143.9 | 0.16 | 0.47 | 0.26894 | 0.56779 |
| Rptor | 1617.52 | 1871.91 | 1873.04 | 1782.92 | 1709.06 | 1528.98 | 1730.6 | −0.04 | −0.09 | 0.58781 | 0.81665 |
| Rtcb | 4607.46 | 4772.03 | 4381.68 | 3327.53 | 3304.38 | 3105.2 | 3916.4 | −0.44 | −0.5 | 0.00033 | 0.00746 |
| Rtel1 | 657.6 | 386.62 | 385.24 | 846.66 | 679.74 | 741.8 | 616.3 | 0.37 | 0.67 | 0.03331 | 0.17437 |
| Sae1 | 3564.69 | 2092.57 | 1737.14 | 4739.53 | 3979.96 | 4556.39 | 3445 | 0.49 | 0.84 | 0.01366 | 0.09953 |
| Samm50 | 2779.85 | 1704.1 | 1834.21 | 2221.94 | 2103.04 | 2243.93 | 2147.8 | 0.02 | 0.06 | 0.83618 | 0.93597 |
| Sdhb | 1750.75 | 1634.56 | 1761.66 | 1997.05 | 1828.37 | 1726.24 | 1783.1 | 0.05 | 0.11 | 0.48645 | 0.75481 |
| Sdhc | 3294.82 | 2712.83 | 2789.64 | 2651.09 | 2448.46 | 2526.39 | 2737.2 | −0.11 | −0.21 | 0.23176 | 0.52589 |
| Sec63 | 5522.12 | 7062.08 | 6304.79 | 4922.3 | 5407.41 | 5028.7 | 5707.9 | −0.18 | −0.3 | 0.10213 | 0.33818 |
| Sepsecs | 828.4 | 1756.94 | 1607.36 | 858.31 | 1100.07 | 781.62 | 1155.5 | −0.27 | −0.61 | 0.09531 | 0.32505 |
| Shoc2 | 3392.18 | 2153.76 | 1841.37 | 3218.22 | 2990.86 | 3282.08 | 2813.1 | 0.15 | 0.36 | 0.23475 | 0.52912 |
| Slc31a1 | 2634.66 | 5959.7 | 3892.21 | 3280.04 | 3483.33 | 3000.55 | 3708.4 | −0.13 | −0.35 | 0.31849 | 0.62003 |
| Slc33a1 | 1830.17 | 2209.39 | 1824 | 2076.79 | 2021.19 | 2134.65 | 2016 | 0.04 | 0.09 | 0.61179 | 0.83207 |
| Slc39a9 | 2984.81 | 3673.36 | 3368 | 2955.71 | 3149.01 | 2744.95 | 3146 | −0.09 | −0.18 | 0.31323 | 0.61548 |
| Slc7a5 | 875.37 | 1130.19 | 1425.48 | 1529.37 | 1319.25 | 1062.23 | 1223.6 | 0.07 | 0.19 | 0.51716 | 0.77498 |
| Snupn | 403.1 | 254.97 | 231.96 | 276.85 | 241.38 | 263.94 | 278.7 | −0.07 | −0.19 | 0.54317 | 0.79051 |
| Sod2 | 1924.97 | 3772.56 | 3592.81 | 2720.97 | 2988.09 | 2388.4 | 2898 | −0.07 | −0.2 | 0.55314 | 0.79657 |
| Spata5 | 657.6 | 523.84 | 449.61 | 765.13 | 782.4 | 847.38 | 671 | 0.38 | 0.55 | 0.01669 | 0.11311 |
| Spata5l1 | 111.02 | 78.81 | 64.38 | 119.16 | 91.56 | 117.61 | 97.1 | 0.15 | 0.37 | 0.26258 | 0.56058 |
| Spcs2 | 4595.5 | 4867.52 | 4641.23 | 7034.93 | 6350.73 | 6577.13 | 5677.8 | 0.44 | 0.5 | 0.00033 | 0.00744 |
| Src | 6345.39 | 3928.32 | 3485.52 | 5132.85 | 3960.54 | 4720.31 | 4595.5 | 0 | 0.01 | 0.98561 | 0.99484 |
| Srp14 | 1647.41 | 1998 | 1732.03 | 1592.98 | 1419.13 | 1397.48 | 1631.2 | −0.17 | −0.29 | 0.11366 | 0.3594 |
| Srp9 | 1292.14 | 1208.07 | 1150.6 | 1383.33 | 1130.59 | 1472.49 | 1272.9 | 0.06 | 0.13 | 0.51546 | 0.77362 |
| Stt3a | 8734.1 | 8377.7 | 7202.99 | 9386.78 | 9195.93 | 9094.26 | 8665.3 | 0.1 | 0.19 | 0.25141 | 0.54866 |
| Stt3b | 7728.06 | 7193.73 | 6671.63 | 8380.64 | 8124.99 | 7467.11 | 7594.4 | 0.08 | 0.15 | 0.34175 | 0.64023 |
| Tada1 | 896.72 | 680.53 | 551.8 | 869.96 | 774.07 | 828.86 | 767 | 0.09 | 0.22 | 0.4102 | 0.69955 |
| Taf1c | 400.54 | 221.59 | 228.89 | 342.25 | 274.67 | 237.08 | 284.2 | 0 | 0 | 0.9897 | 0.99631 |
| Taf2 | 2100.9 | 2077.74 | 1819.91 | 1964.8 | 2048.94 | 2110.57 | 2020.5 | 0.01 | 0.03 | 0.85049 | 0.94145 |
| Tbcb | 1181.97 | 1048.6 | 962.58 | 1758.73 | 1125.04 | 1434.52 | 1251.9 | 0.23 | 0.44 | 0.08893 | 0.3131 |
| Tbce | 731.9 | 598.94 | 679.53 | 663 | 690.84 | 653.82 | 669.7 | 0 | 0 | 0.98832 | 0.9958 |
| Tbp | 612.33 | 569.27 | 482.31 | 680.92 | 632.58 | 633.45 | 601.8 | 0.12 | 0.23 | 0.23544 | 0.52978 |
| Tead1 | 3453.67 | 4858.25 | 3945.35 | 5414.18 | 4866.4 | 4937.01 | 4579.1 | 0.17 | 0.31 | 0.14553 | 0.4116 |
| Telo2 | 446.65 | 345.83 | 293.27 | 461.41 | 371.78 | 353.77 | 378.8 | 0.05 | 0.13 | 0.63565 | 0.84481 |
| Ten1 | 529.49 | 432.98 | 376.04 | 387.05 | 339.87 | 326.91 | 398.7 | −0.18 | −0.35 | 0.14145 | 0.40624 |
| Tex10 | 1330.57 | 890.06 | 731.64 | 1436.19 | 1298.45 | 1570.66 | 1209.6 | 0.28 | 0.54 | 0.07291 | 0.27815 |
| Tfb2m | 280.12 | 230.86 | 268.75 | 250.86 | 289.93 | 281.53 | 267 | 0.03 | 0.07 | 0.71447 | 0.88392 |
| Tfrc | 3220.52 | 7872.4 | 8932.98 | 9858.94 | 13420.04 | 9494.33 | 8799.9 | 0.27 | 0.71 | 0.10398 | 0.34135 |
| Thg1l | 297.2 | 207.68 | 192.11 | 268.78 | 245.54 | 258.38 | 244.9 | 0.06 | 0.15 | 0.58079 | 0.81251 |
| Thoc6 | 428.72 | 283.71 | 320.86 | 381.67 | 310.74 | 342.66 | 344.7 | 0 | 0 | 0.99036 | 0.99678 |
| Timm10 | 327.09 | 220.66 | 212.54 | 355.69 | 306.58 | 354.69 | 296.2 | 0.21 | 0.42 | 0.12592 | 0.37998 |
| Timm22 | 785.7 | 620.26 | 524.21 | 697.94 | 568.76 | 627.89 | 637.5 | −0.01 | −0.03 | 0.91515 | 0.96928 |
| Tmem165 | 2736.29 | 3161.57 | 2843.8 | 3300.65 | 3577.66 | 3304.31 | 3154 | 0.13 | 0.22 | 0.16029 | 0.43388 |
| Toe1 | 697.74 | 469.14 | 555.88 | 622.68 | 585.41 | 569.55 | 583.4 | 0.02 | 0.04 | 0.84906 | 0.94078 |
| Tomm40 | 1393.77 | 905.82 | 757.19 | 2602.71 | 1878.31 | 2310.61 | 1641.4 | 0.86 | 1.15 | 0.00037 | 0.00821 |
| Tpi1 | 19814.2 | 7866.84 | 6812.65 | 13563.66 | 10569.29 | 13660.83 | 12047.9 | 0.04 | 0.13 | 0.7792 | 0.91234 |
| Trappc1 | 709.69 | 700 | 615.15 | 703.31 | 621.48 | 694.57 | 674 | 0 | 0 | 0.98575 | 0.99486 |
| Trappc3 | 2001.83 | 1480.65 | 1555.25 | 1403.94 | 1428.84 | 1466.94 | 1556.2 | −0.11 | −0.23 | 0.26833 | 0.56758 |
| Trit1 | 400.54 | 282.78 | 283.05 | 281.33 | 327.39 | 293.57 | 311.4 | −0.04 | −0.1 | 0.68736 | 0.87032 |
| Trmt61a | 342.46 | 141.85 | 171.67 | 469.47 | 446.69 | 457.49 | 338.3 | 0.6 | 1.07 | 0.00758 | 0.06832 |
| Tsen2 | 167.39 | 134.44 | 118.53 | 165.75 | 142.88 | 140.77 | 145 | 0.04 | 0.1 | 0.71062 | 0.88212 |
| Tsen54 | 311.72 | 242.91 | 222.76 | 275.05 | 155.37 | 203.74 | 235.3 | −0.11 | −0.29 | 0.3657 | 0.66256 |
| Txn2 | 1680.72 | 1752.31 | 1706.48 | 2193.27 | 1940.73 | 1860.53 | 1855.7 | 0.13 | 0.22 | 0.16275 | 0.43728 |
| Tyms | 1047.03 | 484.9 | 320.86 | 1558.04 | 1159.72 | 1456.75 | 1004.6 | 0.55 | 1.17 | 0.01416 | 0.10188 |
| Ugp2 | 2790.09 | 3476.8 | 3315.89 | 3472.67 | 4049.32 | 3261.71 | 3394.4 | 0.08 | 0.17 | 0.39716 | 0.69018 |
| Urb1 | 930.89 | 556.29 | 479.25 | 1050.04 | 961.35 | 1002.96 | 830.1 | 0.31 | 0.62 | 0.05861 | 0.24383 |
| Urm1 | 733.61 | 575.76 | 490.49 | 655.83 | 506.34 | 579.74 | 590.3 | −0.02 | −0.05 | 0.85622 | 0.94473 |
| Utp23 | 342.46 | 324.5 | 261.59 | 356.58 | 357.9 | 342.66 | 331 | 0.09 | 0.19 | 0.37084 | 0.66763 |
| Vhl | 721.65 | 1115.36 | 921.71 | 816.2 | 699.16 | 702.91 | 829.5 | −0.15 | −0.31 | 0.21283 | 0.5038 |
| Vma21 | 1232.36 | 1519.59 | 1336.57 | 1456.8 | 1427.46 | 1571.58 | 1424.1 | 0.06 | 0.12 | 0.48328 | 0.75273 |
| Vmp1 | 12227.91 | 8718.89 | 7032.35 | 8611.79 | 7678.31 | 8888.66 | 8859.7 | −0.06 | −0.15 | 0.58926 | 0.81803 |
| Vps29 | 1426.22 | 1515.89 | 1537.88 | 1564.31 | 1492.66 | 1565.1 | 1517 | 0.02 | 0.05 | 0.74577 | 0.8981 |
| Vps45 | 640.52 | 644.37 | 530.34 | 550.11 | 600.67 | 581.59 | 591.3 | −0.03 | −0.07 | 0.71157 | 0.88239 |
| Vrk1 | 687.49 | 487.68 | 451.66 | 908.48 | 761.59 | 793.66 | 681.8 | 0.38 | 0.6 | 0.02093 | 0.13006 |
| Wars2 | 396.27 | 389.4 | 349.47 | 287.6 | 348.19 | 288.94 | 343.3 | −0.17 | −0.3 | 0.13276 | 0.39106 |
| Wdr18 | 1143.54 | 931.78 | 726.53 | 1215.79 | 910.02 | 1020.56 | 991.4 | 0.07 | 0.17 | 0.53757 | 0.78717 |
| Wdr25 | 124.69 | 128.87 | 117.51 | 101.24 | 152.6 | 109.28 | 122.4 | −0.02 | −0.04 | 0.88296 | 0.95679 |
| Wdr61 | 2459.59 | 1350.85 | 1310.01 | 1694.23 | 1654.96 | 1768.84 | 1706.4 | 0 | 0 | 0.99799 | 0.99933 |
| Wdr7 | 1290.43 | 1957.21 | 2797.81 | 1772.17 | 2287.54 | 1642.89 | 1958 | −0.03 | −0.08 | 0.81527 | 0.9273 |
| Wdr77 | 981.27 | 1156.15 | 715.29 | 1199.67 | 997.42 | 926.1 | 996 | 0.05 | 0.13 | 0.63539 | 0.84481 |
| Wwtr1 | 4480.21 | 4293.62 | 4067.97 | 4104.31 | 3644.25 | 4109.09 | 4116.6 | −0.06 | −0.11 | 0.45351 | 0.732 |
| Xylt2 | 612.33 | 432.98 | 478.22 | 442.6 | 323.22 | 348.21 | 439.6 | −0.24 | −0.45 | 0.08684 | 0.30869 |
| Yars | 1258.83 | 954.03 | 1021.85 | 1437.99 | 1324.8 | 1221.52 | 1203.2 | 0.16 | 0.3 | 0.14949 | 0.41823 |
| Znrd1 | 469.71 | 461.72 | 358.67 | 452.45 | 319.06 | 359.33 | 403.5 | −0.08 | −0.19 | 0.46049 | 0.73689 |

Other mice, whose tumors recurred after prolonged MRTX-849 treatment, were treated with MRTX849 (100 mg/kg/d) plus the tool SHP2 inhibitor SHP099 (75 mg/kg/d). Addition of SHP099 resulted in partial responses, but tumors resistant to both agents (Combo-resistant tumors) soon recurred (FIG. 6A). RNA-seq of nodules from these tumors also revealed pathways common to the CRISPR/Cas9 SL genes (FIG. 6D, starred in red). For example, genes annotated as YAP and WWTR1-stimulated were induced, as were those associated with regulation of RHO activity and O-linked glycosylation (FIG. 6D; FIG. 13B). Other pathways were only associated with resistance to MRTX-849 (e.g., interferon signaling, VEGF signaling and others), or MRTX/SHP099-treatment (e.g., degradation of cysteine and homocysteine, PI3K events in ERBB2, PPAR signaling, and others) groups, respectively. Notably, TEAD and/or TAZ were also induced in RPPAs of nodules from MRTX-849/SHP099-treated mice (FIG. 6E).

Finally, sc-RNAseq data from tumor samples from two NSCLC patients with KRAS^{G12C} mutant tumors were analyzed: one developed resistance to sotorasib (AMG-510) and another whose tumor was resistant to MRTX-849+TNO-155. These data were compared with scRNAseq of a KRAS^{G12V}-mutant NSCLC (FIG. 6F-G, Table 7). Remarkably, several shared resistance pathways were seen in the CRISPR/Cas9 screens as well as the acquired resistant GEMM data: Hippo and RHO GTPase signaling genes were enriched in both resistant tumors, along with glycolysis, MYC, glycosylation and mTOR signaling genes. Other pathways, including genes associated with epithelial-mesenchymal transition, Krebs cycle, DNA damage, KEAP1-NFE2L2 pathways, and autophagy, also were enriched in treated tumors.

TABLE 7
Differential gene expression analysis of scRNA-seq data
from tumor cluster in patient #1778 (AMG-510-resistant tumor)
vs patient #1566 (control, KRAS^{G12V} tumor) showing
RHO genes and any synthetic lethal genes from MRTX-849
CRISPR/Cas9 screens that overlap in 2 or more lines.

| id | p_val | avg_log2FC | pct.1 | pct.2 | p_val_adj |
|---|---|---|---|---|---|
| GAPDH | 8.19E−111 | 2.05724995 | 0.977 | 0.918 | 2.60E−106 |
| MCL1 | 3.72E−90 | −1.8104747 | 0.349 | 0.771 | 1.18E−85 |
| VMP1 | 6.42E−62 | −1.2949166 | 0.419 | 0.749 | 2.04E−57 |
| COX17 | 1.32E−52 | 1.33741553 | 0.761 | 0.513 | 4.19E−48 |
| ELP5 | 3.86E−48 | 0.73755551 | 0.435 | 0.163 | 1.23E−43 |
| TPI1 | 2.58E−47 | 1.09347394 | 0.89 | 0.739 | 8.22E−43 |
| MRPS24 | 3.57E−38 | 0.6914289 | 0.383 | 0.161 | 1.13E−33 |
| SOD2 | 5.19E−37 | −1.0389453 | 0.284 | 0.598 | 1.65E−32 |
| EIF3H | 9.83E−33 | 0.81891842 | 0.813 | 0.611 | 3.13E−28 |
| MRPS6 | 7.50E−32 | −1.0914524 | 0.417 | 0.649 | 2.38E−27 |
| SPCS2 | 2.41E−29 | −0.7919293 | 0.554 | 0.704 | 7.65E−25 |
| PDAP1 | 2.51E−27 | 0.86434926 | 0.628 | 0.436 | 7.99E−23 |
| ERBB3 | 1.19E−26 | −0.6388877 | 0.18 | 0.437 | 3.77E−22 |
| ARL2 | 1.91E−24 | 0.82825304 | 0.55 | 0.373 | 6.08E−20 |
| RHOB | 1.38E−23 | −0.6878454 | 0.538 | 0.714 | 4.40E−19 |
| CCT4 | 1.99E−23 | 0.62387349 | 0.651 | 0.479 | 6.32E−19 |
| IL6ST | 1.50E−22 | −0.7486937 | 0.32 | 0.525 | 4.78E−18 |
| RPL14 | 7.77E−21 | 0.46115282 | 0.964 | 0.942 | 2.47E−16 |
| DSCC1 | 1.96E−18 | 0.2614922 | 0.149 | 0.042 | 6.22E−14 |
| NARS | 2.90E−17 | 0.66566794 | 0.547 | 0.414 | 9.21E−13 |
| VMA21 | 3.62E−17 | 0.58295031 | 0.45 | 0.317 | 1.15E−12 |
| ALG9 | 4.69E−16 | 0.45722384 | 0.173 | 0.063 | 1.49E−11 |
| VPS29 | 6.74E−15 | −0.6223807 | 0.486 | 0.625 | 2.14E−10 |
| HNF1B | 1.04E−14 | −0.1170413 | 0.02 | 0.16 | 3.29E−10 |
| TYMS | 1.14E−14 | 0.54055777 | 0.279 | 0.138 | 3.63E−10 |
| RHOU | 3.39E−14 | −0.2468875 | 0.14 | 0.339 | 1.08E−09 |
| MRPL47 | 3.62E−14 | 0.5418562 | 0.491 | 0.372 | 1.15E−09 |
| SRP14 | 4.30E−14 | −0.3353719 | 0.822 | 0.861 | 1.37E−09 |
| RHOF | 1.10E−13 | −0.2987577 | 0.128 | 0.313 | 3.51E−09 |
| CDIPT | 2.68E−13 | −0.3473392 | 0.207 | 0.388 | 8.53E−09 |
| GUK1 | 2.07E−12 | −0.3701619 | 0.705 | 0.697 | 6.58E−08 |
| TMEM165 | 4.19E−12 | −0.3880483 | 0.342 | 0.508 | 1.33E−07 |
| COPS6 | 5.15E−12 | 0.44276214 | 0.545 | 0.432 | 1.64E−07 |
| RPP21 | 2.97E−11 | 0.4439975 | 0.396 | 0.294 | 9.44E−07 |
| GMPS | 3.98E−11 | 0.45843467 | 0.399 | 0.294 | 1.26E−06 |
| LSM10 | 6.79E−11 | −0.3336857 | 0.239 | 0.4 | 2.16E−06 |
| TRAPPC3 | 8.19E−11 | −0.4480729 | 0.302 | 0.452 | 2.60E−06 |
| AHCYL1 | 1.39E−10 | −0.4542356 | 0.313 | 0.45 | 4.43E−06 |
| POLR3K | 2.47E−10 | −0.2885394 | 0.234 | 0.397 | 7.84E−06 |
| SLC33A1 | 2.63E−10 | −0.2127078 | 0.113 | 0.259 | 8.38E−06 |
| ASNA1 | 5.19E−10 | −0.3539468 | 0.257 | 0.41 | 1.65E−05 |
| NCAPD2 | 8.68E−10 | 0.27600765 | 0.182 | 0.092 | 2.76E−05 |
| POP5 | 3.91E−09 | −0.3257246 | 0.232 | 0.39 | 0.0001242 |
| CD3EAP | 5.20E−09 | −0.0445882 | 0.117 | 0.258 | 0.00016517 |
| ZNRD1 | 5.72E−09 | −0.2021899 | 0.23 | 0.391 | 0.00018198 |
| ROCK2 | 1.19E−08 | 0.45723329 | 0.392 | 0.312 | 0.00037718 |
| GFPT1 | 1.36E−08 | 0.59970868 | 0.516 | 0.422 | 0.00043304 |
| MVD | 1.67E−08 | −0.2022042 | 0.113 | 0.241 | 0.00053057 |
| RABIF | 1.93E−08 | −0.1966501 | 0.162 | 0.306 | 0.00061316 |
| SRC | 2.81E−08 | −0.0762137 | 0.086 | 0.204 | 0.00089318 |
| BPTF | 5.05E−08 | −0.3564765 | 0.376 | 0.483 | 0.00160603 |
| AP2S1 | 5.85E−08 | −0.353644 | 0.545 | 0.62 | 0.0018604 |
| ARMC7 | 7.88E−08 | −0.0672063 | 0.059 | 0.162 | 0.00250562 |
| EXT1 | 8.70E−08 | −0.0839161 | 0.108 | 0.232 | 0.0027659 |
| RNMT | 1.14E−07 | 0.55581917 | 0.453 | 0.384 | 0.00361516 |
| MCMBP | 1.25E−07 | 0.33562573 | 0.304 | 0.223 | 0.00395825 |
| N6AMT1 | 1.27E−07 | −0.1048641 | 0.077 | 0.185 | 0.00405052 |
| TFB2M | 1.28E−07 | −0.2112541 | 0.189 | 0.326 | 0.00405669 |
| EMC1 | 2.89E−07 | −0.109955 | 0.122 | 0.243 | 0.00917986 |
| MPI | 3.40E−07 | −0.0855537 | 0.081 | 0.188 | 0.01082068 |
| JMJD6 | 6.34E−07 | −0.3019205 | 0.227 | 0.365 | 0.02016174 |
| NOP56 | 6.55E−07 | 0.28666047 | 0.41 | 0.34 | 0.02083816 |
| TEN1 | 7.30E−07 | −0.1519536 | 0.173 | 0.306 | 0.02321035 |
| VRK1 | 7.98E−07 | 0.28190933 | 0.18 | 0.106 | 0.02536611 |
| RPTOR | 1.02E−06 | −0.0323845 | 0.054 | 0.143 | 0.03233591 |
| TAF1C | 1.18E−06 | −0.1603331 | 0.056 | 0.143 | 0.03742308 |
| MRPL4 | 1.26E−06 | −0.3149492 | 0.331 | 0.447 | 0.04009988 |
| DDX51 | 1.40E−06 | −0.0883222 | 0.072 | 0.167 | 0.0445194 |
| PSMG4 | 1.42E−06 | −0.1841585 | 0.187 | 0.313 | 0.04514684 |
| DKC1 | 1.49E−06 | 0.43903757 | 0.36 | 0.301 | 0.047302 |
| EXT2 | 1.81E−06 | −0.0320085 | 0.097 | 0.203 | 0.05753641 |
| PMPCB | 2.47E−06 | 0.31536313 | 0.432 | 0.37 | 0.07854697 |
| RHOC | 2.54E−06 | −0.2935239 | 0.349 | 0.465 | 0.08080589 |
| ROCK1 | 2.55E−06 | −0.3628805 | 0.313 | 0.437 | 0.08112817 |
| ACTR6 | 5.50E−06 | 0.28705189 | 0.286 | 0.222 | 0.17476059 |
| THG1L | 7.78E−06 | −0.1065399 | 0.086 | 0.176 | 0.24737804 |
| ERCC1 | 8.37E−06 | −0.2633344 | 0.255 | 0.382 | 0.26603455 |
| PAK2 | 8.41E−06 | 0.25580793 | 0.5 | 0.435 | 0.26730312 |
| RABGGTB | 1.02E−05 | 0.25922371 | 0.396 | 0.336 | 0.32365609 |
| ARMC5 | 1.05E−05 | −0.0676113 | 0.054 | 0.131 | 0.33496274 |
| TRAPPC1 | 1.34E−05 | 0.22437223 | 0.525 | 0.449 | 0.42730992 |
| SAMM50 | 1.52E−05 | −0.1100936 | 0.185 | 0.298 | 0.48257853 |
| FDXR | 1.64E−05 | 0.00955315 | 0.11 | 0.211 | 0.52109166 |
| IMP3 | 1.82E−05 | −0.3240713 | 0.295 | 0.415 | 0.5800274 |
| PHF12 | 1.88E−05 | −0.1145001 | 0.144 | 0.248 | 0.59799194 |
| MOGS | 1.97E−05 | −0.2156815 | 0.18 | 0.293 | 0.62655226 |
| HNRNPU | 1.99E−05 | −0.2474881 | 0.671 | 0.663 | 0.63352105 |
| SRP9 | 2.08E−05 | −0.1687006 | 0.732 | 0.729 | 0.66267965 |
| KDM2A | 2.09E−05 | −0.2813018 | 0.282 | 0.396 | 0.6634571 |
| KTI12 | 2.34E−05 | −0.0863917 | 0.05 | 0.12 | 0.74262939 |
| ILF2 | 2.44E−05 | 0.33149184 | 0.644 | 0.551 | 0.77611132 |
| MTG2 | 4.95E−05 | −0.1594299 | 0.097 | 0.183 | 1 |
| MOB4 | 5.33E−05 | 0.27424554 | 0.396 | 0.352 | 1 |
| NUDC | 5.96E−05 | 0.32120353 | 0.568 | 0.465 | 1 |
| LARS | 6.09E−05 | 0.28336819 | 0.306 | 0.254 | 1 |
| RAD51D | 6.62E−05 | −0.0793453 | 0.043 | 0.105 | 1 |
| TAF2 | 6.69E−05 | 0.27391832 | 0.27 | 0.215 | 1 |
| SLC7A5 | 6.76E−05 | 0.52830577 | 0.27 | 0.221 | 1 |
| GMPPB | 7.25E−05 | −0.0916222 | 0.164 | 0.267 | 1 |
| RIC8A | 7.39E−05 | −0.096491 | 0.2 | 0.313 | 1 |
| NAA20 | 7.50E−05 | 0.09216117 | 0.464 | 0.416 | 1 |
| GRB2 | 7.92E−05 | −0.2394722 | 0.369 | 0.47 | 1 |
| CENPN | 8.07E−05 | 0.29877143 | 0.151 | 0.096 | 1 |
| PPP4C | 9.63E−05 | 0.20514736 | 0.534 | 0.469 | 1 |
| ALG1 | 0.00010778 | −0.0645042 | 0.113 | 0.201 | 1 |
| GFM1 | 0.00014999 | −0.1570061 | 0.187 | 0.28 | 1 |
| DNAJC9 | 0.00016237 | 0.31803448 | 0.297 | 0.242 | 1 |
| SHOC2 | 0.00016331 | 0.35496091 | 0.363 | 0.329 | 1 |
| PRMT1 | 0.00016733 | 0.24728576 | 0.55 | 0.472 | 1 |
| IPO9 | 0.00018203 | 0.03655375 | 0.178 | 0.28 | 1 |
| RHOG | 0.00018221 | −0.2482375 | 0.194 | 0.292 | 1 |
| CLTC | 0.00020662 | −0.2340741 | 0.486 | 0.535 | 1 |
| CUL2 | 0.00023022 | 0.29856164 | 0.275 | 0.229 | 1 |
| DNAJC17 | 0.00026682 | −0.0462793 | 0.167 | 0.264 | 1 |
| RAB10 | 0.0002883 | 0.19279113 | 0.489 | 0.44 | 1 |
| NSMCE2 | 0.00035067 | 0.19578232 | 0.34 | 0.3 | 1 |
| IPO11 | 0.00039707 | 0.11158451 | 0.155 | 0.105 | 1 |
| MRPS34 | 0.00042664 | −0.3124726 | 0.486 | 0.52 | 1 |
| MRPL21 | 0.00043943 | 0.11567767 | 0.439 | 0.393 | 1 |
| BCCIP | 0.00045643 | 0.20661383 | 0.36 | 0.322 | 1 |
| WDR61 | 0.00056567 | −0.1551002 | 0.239 | 0.341 | 1 |
| ERCC2 | 0.00058164 | −0.0721665 | 0.081 | 0.147 | 1 |
| PGD | 0.00060794 | 0.13647359 | 0.239 | 0.185 | 1 |
| NHP2 | 0.00067449 | 0.22058213 | 0.52 | 0.456 | 1 |
| CHMP7 | 0.00080082 | −0.0241092 | 0.128 | 0.21 | 1 |
| PDSS2 | 0.00087214 | 0.12584985 | 0.191 | 0.142 | 1 |
| STT3A | 0.00094196 | −0.1494106 | 0.259 | 0.356 | 1 |
| SNUPN | 0.00104504 | −0.0234606 | 0.162 | 0.246 | 1 |
| WWTR1 | 0.00106373 | 0.34356818 | 0.257 | 0.218 | 1 |
| ALG5 | 0.00112452 | 0.16415146 | 0.378 | 0.352 | 1 |
| DBR1 | 0.00125396 | −0.0393545 | 0.072 | 0.13 | 1 |
| GPN2 | 0.0014109 | −0.0213193 | 0.128 | 0.207 | 1 |
| DOHH | 0.00156327 | 0.0202039 | 0.122 | 0.198 | 1 |
| NAA25 | 0.00158887 | 0.01475608 | 0.162 | 0.245 | 1 |
| GGNBP2 | 0.00184439 | 0.25623421 | 0.432 | 0.402 | 1 |
| OGFR | 0.00184999 | −0.0231374 | 0.133 | 0.205 | 1 |
| CINP | 0.001932 | −0.0065219 | 0.216 | 0.311 | 1 |
| ATP6V1D | 0.00195779 | 0.32350101 | 0.396 | 0.364 | 1 |
| NDST1 | 0.00205181 | −0.0693797 | 0.061 | 0.113 | 1 |
| ELP3 | 0.00287505 | 0.01762572 | 0.106 | 0.173 | 1 |
| ARF1 | 0.00312407 | 0.31397487 | 0.768 | 0.626 | 1 |
| MTOR | 0.00335802 | −0.0667236 | 0.088 | 0.144 | 1 |
| IBA57 | 0.00340982 | −0.0423836 | 0.054 | 0.101 | 1 |
| ALG13 | 0.00341788 | −0.1758691 | 0.322 | 0.421 | 1 |
| FBL | 0.00360989 | 0.24605816 | 0.455 | 0.415 | 1 |
| EXOSC10 | 0.00446672 | 0.10144227 | 0.171 | 0.129 | 1 |
| TELO2 | 0.00464915 | 0.00276424 | 0.108 | 0.17 | 1 |
| ACTR2 | 0.00493221 | 0.17174367 | 0.595 | 0.542 | 1 |
| EXOC2 | 0.00496808 | −0.0357357 | 0.092 | 0.149 | 1 |
| GNB1L | 0.00585157 | 0.01520853 | 0.056 | 0.101 | 1 |
| POLRMT | 0.00636156 | −0.0385534 | 0.117 | 0.173 | 1 |
| ARPC4 | 0.00762597 | 0.088146 | 0.468 | 0.445 | 1 |
| HSD17B10 | 0.00772705 | 0.21870062 | 0.435 | 0.413 | 1 |
| CHTF8 | 0.00818056 | 0.01472781 | 0.198 | 0.277 | 1 |
| GRWD1 | 0.00910812 | 0.1361683 | 0.151 | 0.222 | 1 |
| DPAGT1 | 0.00962462 | −0.1153597 | 0.18 | 0.256 | 1 |
| COX11 | 0.00994819 | 0.14894518 | 0.358 | 0.337 | 1 |
| PTBP1 | 0.01017355 | 0.20781019 | 0.432 | 0.41 | 1 |
| PPP6C | 0.01059635 | 0.08239749 | 0.279 | 0.249 | 1 |
| DYRK1A | 0.0111204 | −0.1052309 | 0.191 | 0.263 | 1 |
| TBP | 0.01134524 | 0.05503914 | 0.11 | 0.076 | 1 |
| TSEN54 | 0.01332224 | −0.0796783 | 0.171 | 0.243 | 1 |
| PSMG1 | 0.01402177 | 0.21693422 | 0.336 | 0.313 | 1 |
| ENO1 | 0.01477109 | 0.03864747 | 0.824 | 0.79 | 1 |
| MRPL28 | 0.01528922 | 0.22449382 | 0.376 | 0.359 | 1 |
| UGP2 | 0.01587528 | 0.13430368 | 0.547 | 0.498 | 1 |
| FARS2 | 0.01632562 | −0.0918806 | 0.113 | 0.164 | 1 |
| PTCD3 | 0.01778282 | 0.06563687 | 0.286 | 0.261 | 1 |
| YARS | 0.01790768 | −0.020829 | 0.255 | 0.333 | 1 |
| RPE | 0.01892659 | −0.1110931 | 0.18 | 0.246 | 1 |
| MRPS18A | 0.01908746 | 0.24838981 | 0.327 | 0.31 | 1 |
| TADA1 | 0.01943997 | −0.0437725 | 0.072 | 0.113 | 1 |
| URB1 | 0.01954001 | 0.05843257 | 0.16 | 0.227 | 1 |
| URM1 | 0.0207238 | 0.14571192 | 0.227 | 0.196 | 1 |
| WDR7 | 0.02124168 | 0.02191372 | 0.068 | 0.107 | 1 |
| GTF3C1 | 0.02273222 | 0.00566334 | 0.187 | 0.258 | 1 |
| NOL9 | 0.02397456 | −0.0167999 | 0.14 | 0.198 | 1 |
| XYLT2 | 0.02478133 | 0.03037519 | 0.126 | 0.18 | 1 |
| THOC6 | 0.02579631 | −0.0600258 | 0.178 | 0.244 | 1 |
| RHOD | 0.02823662 | −0.0337326 | 0.255 | 0.327 | 1 |
| CCNC | 0.02930932 | 0.10287201 | 0.417 | 0.393 | 1 |
| WARS2 | 0.02969248 | −0.0558564 | 0.09 | 0.132 | 1 |
| BRK1 | 0.03017192 | −0.3071245 | 0.52 | 0.558 | 1 |
| EARS2 | 0.03426328 | 0.01822242 | 0.086 | 0.126 | 1 |
| TXN2 | 0.03662291 | −0.1917434 | 0.365 | 0.417 | 1 |
| ANKRD49 | 0.03666936 | 0.00231609 | 0.158 | 0.215 | 1 |
| TRIT1 | 0.03722764 | −0.0149156 | 0.108 | 0.151 | 1 |
| TIMM22 | 0.03783026 | 0.15066793 | 0.178 | 0.153 | 1 |
| SDHB | 0.03793559 | −0.101591 | 0.363 | 0.433 | 1 |
| ADNP | 0.03841717 | −0.1228374 | 0.282 | 0.352 | 1 |
| OTUD5 | 0.04009078 | −0.0635711 | 0.189 | 0.251 | 1 |
| DDX59 | 0.04732459 | −0.0245983 | 0.162 | 0.218 | 1 |
| TBCB | 0.04939374 | 0.07108096 | 0.441 | 0.428 | 1 |
| EMC6 | 0.05173389 | 0.14816218 | 0.354 | 0.352 | 1 |
| PGM3 | 0.05365568 | 0.19901653 | 0.293 | 0.285 | 1 |
| RHOV | 0.05384516 | 0.15548504 | 0.086 | 0.125 | 1 |
| SEPSECS | 0.05546739 | −0.0464357 | 0.072 | 0.105 | 1 |
| KRR1 | 0.05859275 | −0.085513 | 0.342 | 0.404 | 1 |
| UTP23 | 0.05931598 | 0.28259404 | 0.288 | 0.283 | 1 |
| NHLRC2 | 0.06071964 | 0.12022402 | 0.255 | 0.244 | 1 |
| PTPMT1 | 0.06207524 | 0.06077975 | 0.331 | 0.384 | 1 |
| B3GNT2 | 0.06459622 | 0.09070294 | 0.203 | 0.183 | 1 |
| HYOU1 | 0.06617653 | 0.03696443 | 0.252 | 0.313 | 1 |
| TRMT61A | 0.07072389 | −0.0222576 | 0.126 | 0.17 | 1 |
| DNAJB11 | 0.07082479 | 0.18439829 | 0.354 | 0.347 | 1 |
| MBTPS2 | 0.07604952 | 0.0852915 | 0.086 | 0.12 | 1 |
| DHPS | 0.07617758 | −0.1484078 | 0.291 | 0.364 | 1 |
| PAICS | 0.07689048 | 0.25177544 | 0.41 | 0.4 | 1 |
| METTL3 | 0.07693563 | 0.04233039 | 0.146 | 0.195 | 1 |
| DDOST | 0.08008057 | −0.1510766 | 0.505 | 0.5 | 1 |
| NAE1 | 0.08327351 | −0.1086189 | 0.322 | 0.383 | 1 |
| GEMIN7 | 0.08663111 | 0.00158832 | 0.169 | 0.221 | 1 |
| BUB3 | 0.09256567 | 0.17444825 | 0.423 | 0.422 | 1 |
| CDK7 | 0.09315701 | 0.02009673 | 0.155 | 0.205 | 1 |
| DHX33 | 0.09606414 | 0.05103336 | 0.126 | 0.105 | 1 |
| MRPL53 | 0.09800022 | 0.19983541 | 0.2 | 0.188 | 1 |
| TFRC | 0.09814851 | −0.0324505 | 0.286 | 0.35 | 1 |
| SPATA5 | 0.10364305 | 0.00739365 | 0.074 | 0.104 | 1 |
| NOC4L | 0.10676087 | 0.17465262 | 0.187 | 0.169 | 1 |
| PKN2 | 0.12346617 | 0.14845435 | 0.304 | 0.311 | 1 |
| RIOK2 | 0.12443417 | 0.0210845 | 0.169 | 0.221 | 1 |
| EIF3F | 0.14026103 | 0.10613416 | 0.579 | 0.538 | 1 |
| ATP6V1B2 | 0.14295142 | −0.1132902 | 0.261 | 0.315 | 1 |
| RPP38 | 0.14450565 | 0.16295804 | 0.261 | 0.261 | 1 |
| SDHC | 0.14653117 | −0.0457434 | 0.525 | 0.5 | 1 |
| MIS18A | 0.16486018 | 0.2117157 | 0.187 | 0.175 | 1 |
| VHL | 0.16652488 | 0.01147435 | 0.131 | 0.169 | 1 |
| GTF2H3 | 0.16819006 | 0.10612867 | 0.234 | 0.229 | 1 |
| TEAD1 | 0.17852355 | 0.09190113 | 0.27 | 0.334 | 1 |
| CHCHD4 | 0.18361607 | 0.01979157 | 0.104 | 0.134 | 1 |
| ALG2 | 0.19668921 | 0.00230831 | 0.207 | 0.257 | 1 |
| EIF3A | 0.20682426 | 0.18131055 | 0.574 | 0.528 | 1 |
| EIF1AD | 0.20770414 | 0.00067807 | 0.162 | 0.201 | 1 |
| MTG1 | 0.21137308 | 0.00394306 | 0.135 | 0.169 | 1 |
| MAD2L2 | 0.21574263 | 0.07678784 | 0.196 | 0.184 | 1 |
| NDUFS2 | 0.21855968 | −0.2307194 | 0.444 | 0.476 | 1 |
| SLC39A9 | 0.21898547 | −0.0705645 | 0.167 | 0.206 | 1 |
| ILF3 | 0.21963967 | −0.0205799 | 0.435 | 0.475 | 1 |
| EXOSC4 | 0.2198283 | 0.2380673 | 0.284 | 0.291 | 1 |
| GGPS1 | 0.22492732 | 0.03714783 | 0.23 | 0.283 | 1 |
| SAE1 | 0.22597178 | 0.1319261 | 0.239 | 0.292 | 1 |
| NELFB | 0.25488837 | −0.0663452 | 0.176 | 0.214 | 1 |
| AIFM1 | 0.25534101 | 0.07429329 | 0.205 | 0.252 | 1 |
| CNOT1 | 0.25787319 | −0.0016437 | 0.297 | 0.354 | 1 |
| NSMCE1 | 0.26283314 | 0.05657951 | 0.32 | 0.332 | 1 |
| TIMM10 | 0.26607298 | 0.0982854 | 0.349 | 0.405 | 1 |
| PAK1IP1 | 0.27539357 | 0.20935217 | 0.248 | 0.258 | 1 |
| DARS2 | 0.27736674 | 0.00781102 | 0.104 | 0.129 | 1 |
| RHOH | 0.30499753 | −0.2009329 | 0.106 | 0.087 | 1 |
| INTS10 | 0.31170664 | −0.0275666 | 0.293 | 0.347 | 1 |
| AHCY | 0.33273195 | 0.02552897 | 0.349 | 0.404 | 1 |
| EIF2B5 | 0.34051792 | 0.02270532 | 0.207 | 0.249 | 1 |
| ASCC3 | 0.34816382 | 0.21638957 | 0.284 | 0.3 | 1 |
| MTX1 | 0.36082824 | 0.12261646 | 0.378 | 0.391 | 1 |
| PTDSS1 | 0.36640557 | 0.00021159 | 0.158 | 0.19 | 1 |
| HUWE1 | 0.36861085 | 0.17961717 | 0.356 | 0.368 | 1 |
| DAP3 | 0.38644835 | −0.1124577 | 0.43 | 0.459 | 1 |
| PDCD5 | 0.41153443 | 0.01478514 | 0.525 | 0.496 | 1 |
| MPDU1 | 0.41956673 | 0.10822403 | 0.218 | 0.223 | 1 |
| ATP6V1E1 | 0.426336 | −0.0129579 | 0.39 | 0.425 | 1 |
| RTCB | 0.4531385 | 0.10423089 | 0.304 | 0.319 | 1 |
| WDR77 | 0.45534725 | 0.09028759 | 0.221 | 0.225 | 1 |
| LEMD2 | 0.47274603 | 0.04693586 | 0.167 | 0.165 | 1 |
| RHOQ | 0.49047354 | 0.02130867 | 0.273 | 0.307 | 1 |
| CPSF1 | 0.49377469 | 0.05062753 | 0.187 | 0.186 | 1 |
| CRK | 0.52208 | 0.0119465 | 0.25 | 0.261 | 1 |
| MMS19 | 0.53196853 | 0.01746895 | 0.182 | 0.21 | 1 |
| NAA10 | 0.55615168 | −0.0253516 | 0.43 | 0.474 | 1 |
| NELFCD | 0.57971046 | 0.10545473 | 0.259 | 0.276 | 1 |
| WDR18 | 0.58200732 | 0.15430937 | 0.23 | 0.246 | 1 |
| STT3B | 0.5876256 | 0.04970918 | 0.295 | 0.337 | 1 |
| HDAC3 | 0.5903423 | 0.00811574 | 0.241 | 0.283 | 1 |
| SEC63 | 0.60979043 | 0.0135337 | 0.358 | 0.395 | 1 |
| CPSF4 | 0.61128423 | 0.08584999 | 0.176 | 0.202 | 1 |
| DDX6 | 0.6323608 | 0.12600298 | 0.399 | 0.411 | 1 |
| AASDHPPT | 0.64018093 | 0.07557989 | 0.286 | 0.332 | 1 |
| SLC31A1 | 0.65077309 | 0.0474097 | 0.189 | 0.197 | 1 |
| ACTR3 | 0.65999492 | −0.1021497 | 0.556 | 0.551 | 1 |
| DEXI | 0.66985448 | 0.05297348 | 0.178 | 0.206 | 1 |
| ATP6V1F | 0.67176218 | −0.02102 | 0.617 | 0.6 | 1 |
| NELFA | 0.68949811 | −0.0038529 | 0.196 | 0.224 | 1 |
| DIS3 | 0.68971978 | 0.23830801 | 0.252 | 0.275 | 1 |
| DTYMK | 0.70212751 | 0.20081627 | 0.286 | 0.305 | 1 |
| PMPCA | 0.78235205 | −0.0218468 | 0.155 | 0.172 | 1 |
| TOMM40 | 0.79095248 | 0.10215318 | 0.286 | 0.329 | 1 |
| RHOA | 0.80216007 | −0.0325215 | 0.716 | 0.665 | 1 |
| PDCD6IP | 0.81020318 | 0.0079896 | 0.327 | 0.356 | 1 |
| FXN | 0.81420524 | 0.02433229 | 0.117 | 0.129 | 1 |
| WDR25 | 0.8626064 | 0.09293027 | 0.115 | 0.121 | 1 |
| CTNNBL1 | 0.90550406 | 0.1094896 | 0.239 | 0.269 | 1 |
| CSTF1 | 0.91223036 | 0.11448231 | 0.142 | 0.155 | 1 |
| COASY | 0.96145454 | 0.05213759 | 0.273 | 0.305 | 1 |
| VPS45 | 0.96820423 | −0.0142621 | 0.144 | 0.153 | 1 |
| RPN1 | 0.97898604 | −0.0795344 | 0.455 | 0.449 | 1 |
| GRPEL1 | 0.98309816 | 0.0238874 | 0.322 | 0.353 | 1 |

### Example 7. Validation of Other Shared SL Genes from CRISPR/Cas9 Screens

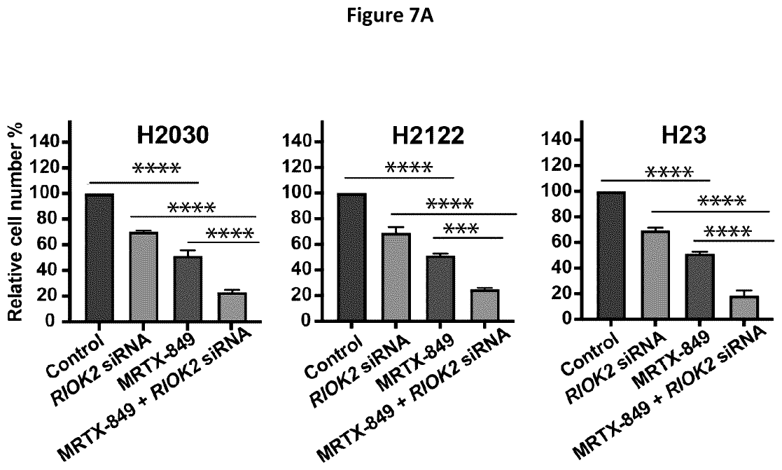

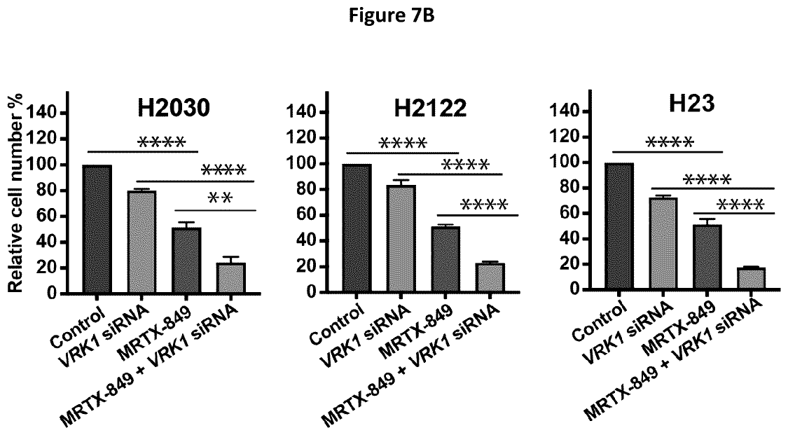

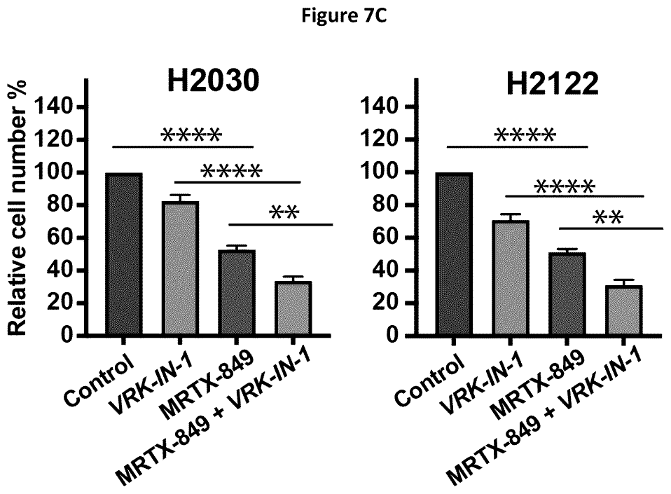

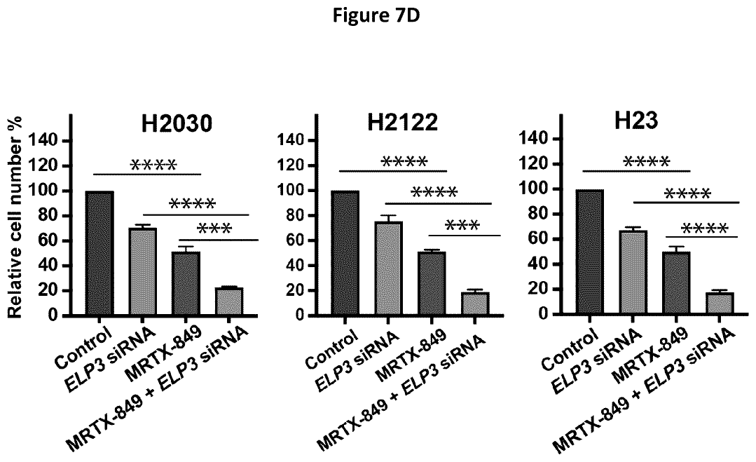

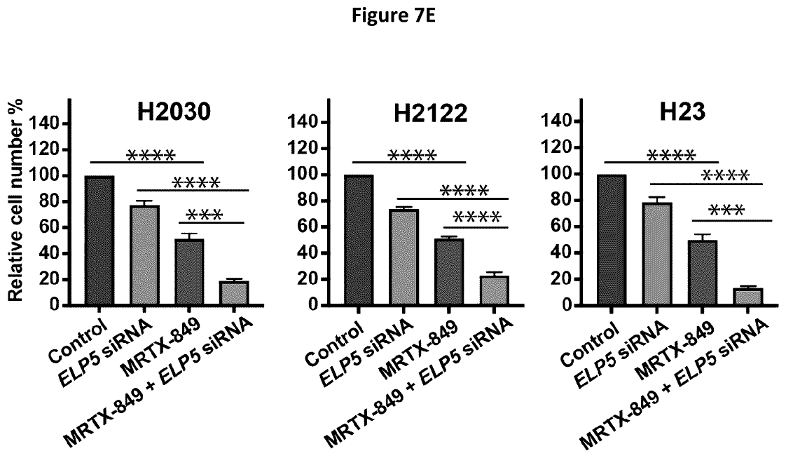

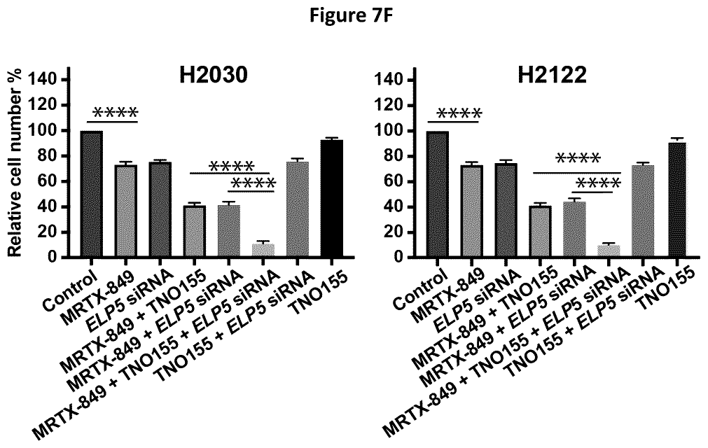

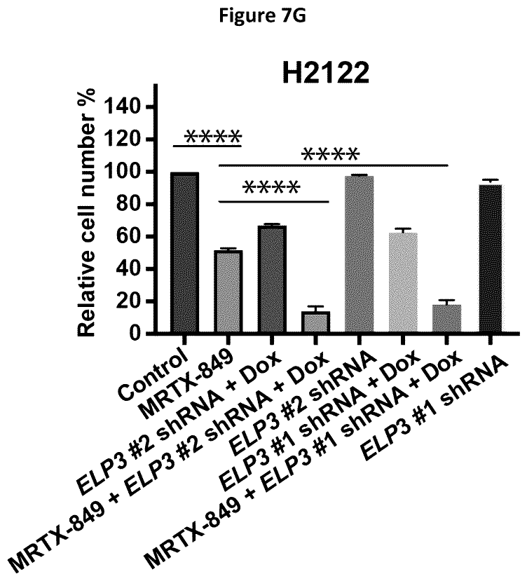

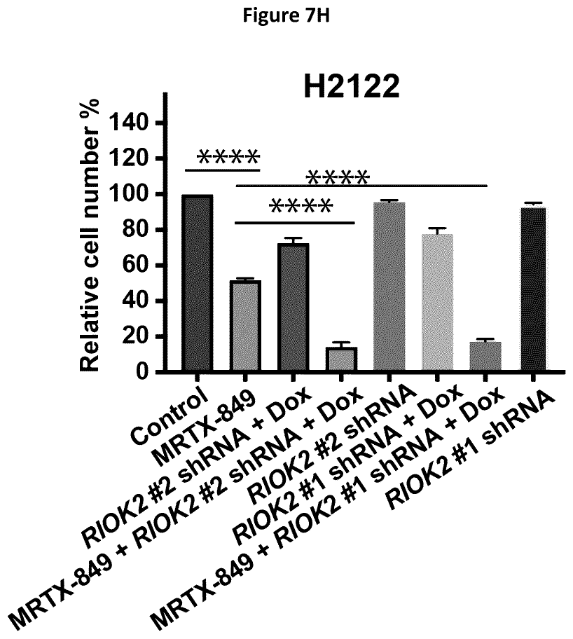

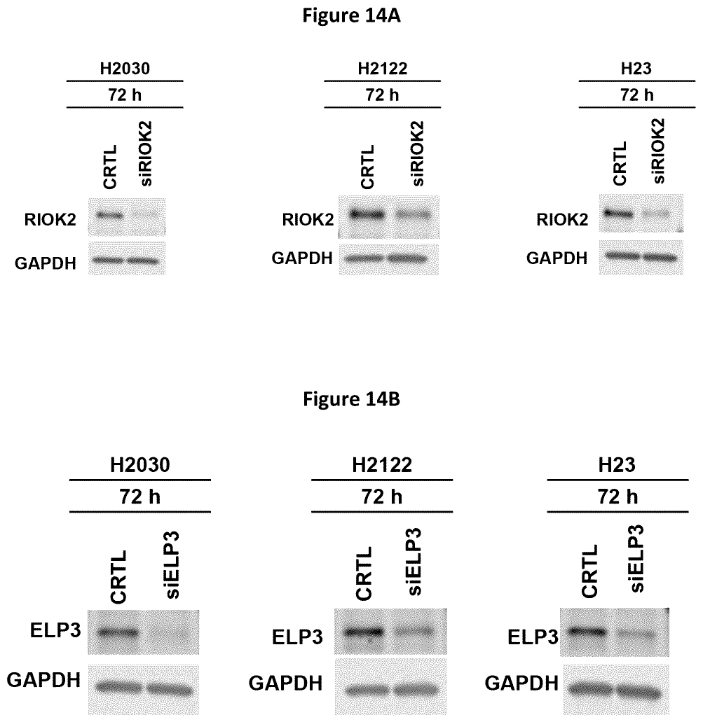

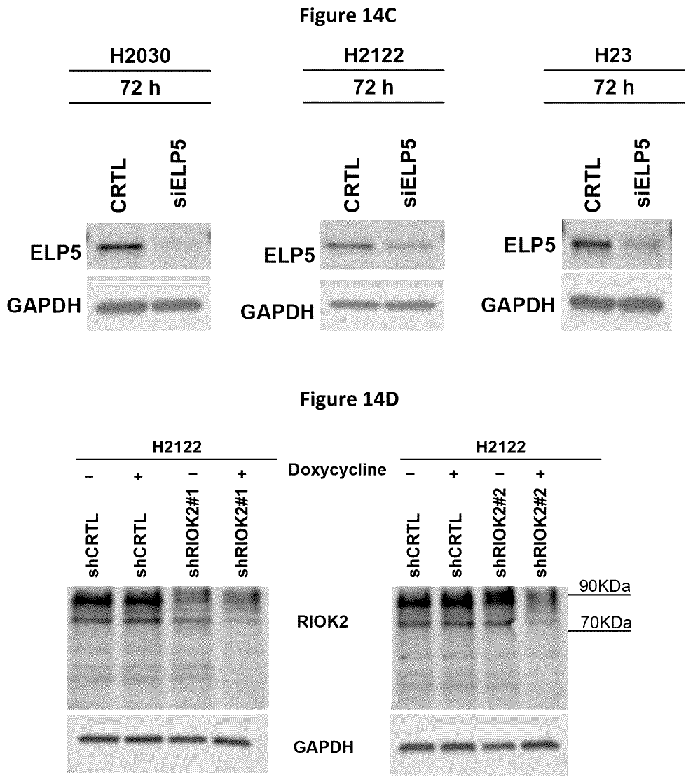

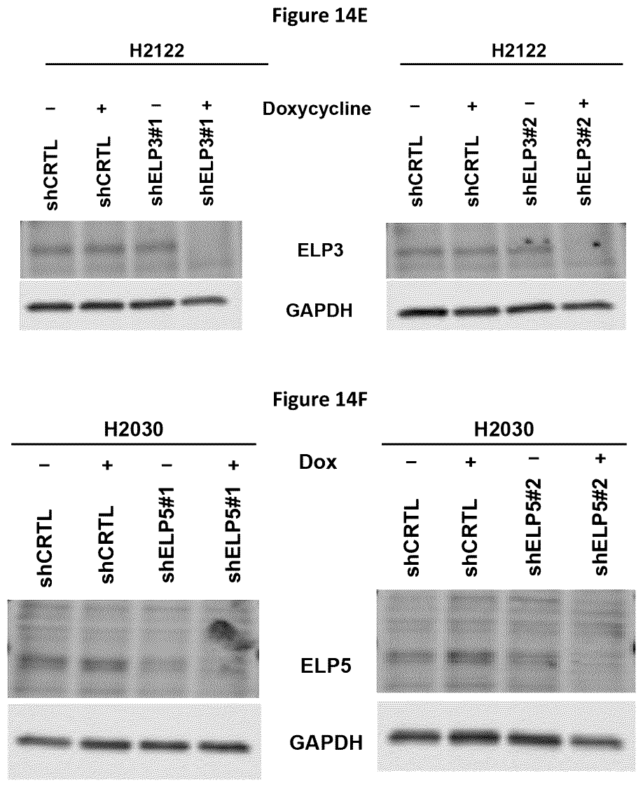

The effects of knockdown of several other recurrent, potentially targetable SL genes were tested. Indeed, siRNAs shRNAs against RIOK2 (FIG. 7A, FIG. 14A) or VRK1 (FIG. 7B), which encode serine/threonine kinases, sensitized multiple NSCLC lines to MRTX-849 (at its IC₅₀). VRK-IN-1(41), a tool VRK1 inhibitor, also enhanced G12Ci efficacy (FIG. 7C). Similar results were obtained with si for ELP3 and ELP5 (FIGS. 7D-7E, FIGS. 14B-14C), which encode components of the elongator complex (42). ELP5 also scored as a significant SL gene in the MRTX-849+TNO-155 screen (FIG. 4A), and ELP5 siRNA enhanced the effect of this combination (FIG. 7F). Similar results were obtained using doxycline-inducible shRNAs (FIGS. 7G-7H, FIGS. 14D-14F). In concert, these data indicate that multiple other genes and pathways besides YAP/TAZ/TEAD could be targeted to enhance G12Ci efficacy.

The development of G12Cis was a major breakthrough in experimental therapeutics, yet their efficacy in the clinic has thus far been modest. The existence of multiple G12Ci resistance mechanisms indicates that combination therapies will be required to maximize the impact of these remarkable drugs. This need is particularly acute for the NSCLC subgroups with co-mutations in STK11 and/or KEAP1, as well as for tumors of other histotypes (e.g., CRC) with KRAS^{G12C} mutations. Identified herein are recurrent synthetic lethal (SL) genes with G12Cis that span a range of functional classes, including genes in pathways related to Hippo and RHO signaling, tRNA processing, and heparan sulfate biosynthesis, as well as several novel kinases. These results provide a landscape of potential new targets for future combination strategies, some of which can be tested rapidly in the clinic, others of which will require new drug development.

Two earlier studies used a similar CRISPR/Cas9 screening approach to search for G12Ci SL genes. Lou et al. (53) surveyed a NSCLC line, H358, and the PDAC line MIAPaCa-2, each maintained in 2D culture and treated with a tool G12Ci (ARS-1620). Han et al. studied H23 cells, which were also used here, maintained in 2D and suspension (3D). The “hits” in the earlier papers reveal some shared dependencies with this study (e.g., ELP3, ELP4, PKN2, RPN1 in the H358 screen; EXT1, WWTR1, SHOC2 in MIAPaCa-2 cells, PGM3 in the 2D component of the 2D vs 3D screen; compare with FIG. 1D with References 53,54). Overall, however, the majority of the shared SL genes discovered herein escaped detection in the prior studies, most likely because of differences in screening conditions, cell systems (including tumor histotype and co-mutations), the number of lines surveyed, and possibly the use of G12Cis at different stages of clinical development. In addition to expanding the landscape of G12Ci resistance genes and pathways, the studies herein also provided insight into resistance to G12Ci/SHP2i combination therapies.

These G12Ci and G12Ci/SHP2i combination screens both identified the YAP/TAZ/TEAD pathway as a route to resistance in vitro and in vivo. These findings were validated genetically and pharmacologically using multiple cell lines, GEMM and CDX models, and two different modes of TEAD inhibition. Importantly, evidence was also found that this pathway is activated in mouse and human models of G12Ci and G12Ci+SHP2i-resistance. These findings comport with previous results implicating the YAP/TAZ/TEAD pathway in resistance to other targeted therapies (29,30) including MEKi and BRAF^{V600E} inhibitors (25,54). Moreover, while this manuscript was in preparation, Hagenbeek et al. (55) reported the development of allosteric TEAD inhibitors and demonstrated their ability to block adaptive resistance to G12Ci treatment, while Adachi et al. (56) found similar effects of YAP1 deficiency in a limited number of NSCLC lines and H358 xenografts. Other TEAD inhibitors are already in phase 1 clinical trials (NCT05228015 and NCT04665206), and preliminary results indicate that at least one, VT3989, is safe with manageable toxicity (19).

It was observed that YAP nuclear translocation and activity are induced by G12Ci treatment. At first glance, this finding was somewhat surprising, because previous work showed that LKB1 (encoded by STK11) antagonizes YAP activation by activating a PAR1->Scribble->MST2->LATs pathway; accordingly STK11−/− cells, as was screened here, should already have significant YAP activation (**Mohseni/Kim). Indeed, consistent with the findings of Mohseni et al., >50% of H2030 and 2122 cells show at least some nuclear localization of YAP (FIG. 3G-H, FIG. 10C-D). This level of YAP activation could contribute to the relative refractoriness of STK11−/− NSCLC to multiple therapeutics (57) but it is clearly inadequate to confer complete G12Ci resistance in vitro or in vivo. Multiple feedback pathways serve to limit YAP/TAZ/TEAD signaling (29) which dampen YAP activation in STK11-deficient cells and help explain this apparent paradox. Alternatively, or in addition, LKB1 activates DBL, a RHO-GEF(58). Hence, RHO levels are likely to be lower in STK11−/−, compared with STK11-replete cells, and thus inadequate to drive sufficient YAP translocation/activation to confer resistance.

Adachi et al. also observed G12Ci-induced YAP translocation in STK11-replete H358 cells and in LU65 cells, which are STK11−/−, but the studies herein differ in mechanistic detail. They reported that G12Ci treatment induces Scribble mislocalization, which leads to decreased MST2/LATS suppression of YAP nuclear localization. Although this model could be particularly relevant for STK11-replete cells, STK11−/− cells should already have mislocalized Scribble (57). By contrast, it was found here that MRTX-849 treatment induces the transcription of genes encoding multiple RHO paralogs and GEFs, as well as RHO targets, actin regulators, and myosins (including myosin-II). RHO, acting though the actomyosin cytoskeleton, is also known to promote YAP activation (29,30). Furthermore, earlier work found that RHOA is necessary for the transformation induced by mutant KRAS (68-70) and for KRAS^{G12D}-driven lung adenocarcinoma (31), while suppressing ERK/MAPK signaling in cancer cells can result in increased RHOA activation (59). Consistent with the observations herein, inhibiting the RHO effector ROCK blocks MRTX-849-induced YAP nuclear translocation and adaptive resistance. The relationship between the MST2/LATS and RHO pathways for YAP activation remains controversial; specifically, it is unclear whether RHO acts via the MST2/YAP pathway or whether the latter is merely permissive for the former (29,30,33). Similarly, it is conceivable that Scribble mislocalization and RHO activation act in parallel in KRAS^{G12C} NSCLC cells; in this regard, it is noted herein that ROCK inhibitor treatment appeared to block YAP translocation only partially (FIG. 3G-H, FIG. 10C-D). Alternatively, another RHO effector (e.g., mDIA, PKN) might play a role in YAP/TEAD pathway activation (FIG. 10E). A possible role for PKN is particularly attractive, as it is also a SL target in the MTRX-849 screen herein.

Future studies are necessary to reveal how this RHO “regulome” is induced by G12Ci treatment, which RHO-GEFs are particularly important for increased RHO activity, and whether other RHO effectors are also important for YAP/TEAD pathway activation. Regardless, the finding herein that Y27632 enhances the effects of MRTX-849 raises the possibility that pharmacologically useful ROCK inhibitors, such as Fasudil, which is approved in Japan and China for the treatment of cerebral vasospasm (60), or others (61,62) might be repurposed for combining with G12Ci.

In addition to the RHO/YAP/TEAD pathway, also validated herein were two kinases (VRK1, RIOK2) and two tRNA-modifying enzymes (ELP3, ELP5) that scored as G12Ci SL genes. VRK1 was previously reported as a “collateral lethality” in glioblastoma, owing to methylation and lack of expression of its paralog VRK2 in this malignancy. VRK1 depletion in these VRK2-deficient cells levels leads to a G2/M phase arrest, followed by DNA damage (63,64). By contrast, VRK2 is expressed in NSCLC. Antibodies capable of detecting VRK2 by immunoblotting could not be identified, but assuming that VRK2 is also expressed at the protein level, VRK1 must have specific functions in G12Ci-treated NSCLC cells. RIOK2 is a relatively unexplored RSK target whose normal function is to promote maturation of the 40S ribosome (65). ELP proteins comprise the so-called “elongator complex”, which catalyzes tRNA modifications under various stresses. Previous research showed that suppression of ELP1 or ELP3 can abrogate vemurafenib in BRAF^{V600E} melanoma (66), and that ELPs contribute to EGFR inhibitor resistance in breast cancer cells by promoting MCL1 synthesis (67). As the elongator complex has acetyl transferase activity, it might, like VRK1 and RIOK2, be amenable to future drug discovery efforts. Furthermore, VRK1, ELP3, and ELP5 expression were significantly increased in the AMG-510-resistant patient sample, while RIOK2 levels were nominally increased (Table 7). Future work will explore the relationship between these and the other SL pathways identified herein and their utility in combination therapy with G12Cis.

Below are the methods used in the Examples described above.

Cell lines and reagents. MIAPaCa-2, Calu-1, H23, H358, H2030, H2122, HCC44, SW1463, and SW837 cells were obtained from laboratory inventories, acquired as reported previously (34). The KCL cell line was derived following an established protocol (43). Briefly, nodules were harvested from lungs with visible tumors in Ad-Cre-induced, KRAS^{LSL-G12C/+}. Stk11^{flox/flox} (KCL) mice on C57BL/6J background (generated as described below) and minced in RPMI 1640 containing 10% Fetal Bovine Serum (FBS, Sigma-Aldrich), 1× GlutaMAX Supplement (Gibco, Cat #: 35050061) and 1× Antibiotic-Antimycotic (Gibco, Cat #: 15240062). The media were exchanged daily, and cells were cultured for at least five passages to establish a stable cell line.

All cultures were maintained at 37° C. in a 5% CO₂ environment using the media conditions specified by the supplier or the originating laboratory. Once thawed, aliquots of cell lines were maintained for no longer than 3 months. TET-ON-shRNA stable cell lines (see below) were cultured in tetracycline-free FBS (Takara Bio). Lines were tested for mycoplasma contamination by PCR 5-7 days after thawing.

MRTX-849 was provided by Mirati Therapeutics under a collaborative research agreement. TNO155, Y27632, and VRK-IN-1 were purchased from MedChemExpress. VT104 and VT106 were provided by Vivace Therapeutics under a collaborative MTA. MYF-03-176 was kindly provided by Dr. Nathanael S. Gray, Stanford University, under a collaborative MTA. SHP099 was purchased from WuXi AppTec (Shanghai) Co., Ltd.

Plasmids, si/shRNAs, and lentivirus generation. To induce TEAD1 (VB230130-1327fbp/VectorBuilder) or WWTR1/TAZ (VB230411-1009nze/VectorBuilder) (over)expression in KCL cells, expression plasmids were generated that fuse the respective coding sequences to 3×-flag tags. These plasmids and a control vector (VB900120-7563srw) were obtained from VectorBuilder. The following constructs were obtained from Addgene: pLX304 (#25890), YAP1 (#42555), YAP1^{S6A} (#42562), and YAPs94A (#59145), dominant negative TEAD and cognate vector control (pInducer20 EGFP-TEADi, #140145, and pInducer20 #44012), and 8×GIITC-luciferase (#34615) and Renilla luciferase (#27163). Stable cell lines overexpressing the indicated genes were generated using lentiviral gene transduction (43).

Doxycycline (Dox)-inducible gene knockdowns were achieved by using TET-ON lentiviral vectors (Tet-pLKO-Puro backbone, Addgene #21915) expressing the appropriate targeting shRNA and a puromycin resistance gene (pLKO-Tet-On-Gene-shRNA1 and -shRNA2). A non-targeting shRNA vector (pLKO-Tet-On-shRNA-Control) served as control. Stable lines were established by puromycin selection for 7 days, and shRNAs were induced by adding Dox (1 g/ml) to the culture medium. Suggested sequences for shRNAs were obtained from the Broad Institute of MIT and were designated TEAD1 #1 (TRCN0000015799), TEAD1 #2 (TRCN0000015800), ELP3 #1 (TRCN0000001280), ELP3 #2 (TRCN0000235508), ELP5 #1 (TRCN0000130483), ELP5 #2 (TRCN0000127506), RIOK2 #1 (TRCN0000197250), and RIOK #2 (TRCN0000196684).

To generate lentiviruses, HEK-293T cells were co-transfected with lentiviral constructs and the packaging plasmids psPAX2 (Addgene, #12260) and pMD2.G (Addgene, #12259) using Lipofectamine 3000 (Invitrogen, Cat #: L3000008) according to the manufacturer's instructions. After 48h, culture media were passed through a 0.45 mm filter (Corning, cat #: 431225) to remove cell debris, and supernatants containing viral particles, supplemented with 8 μg/ml of polybrene (Fisher Scientific, cat #: TR1003G), were used to infect 70% confluent cells in 6-well plates for 16 h at 37° C.

For siRNA experiments, cells were plated in 6-well plates at 30% confluence in medium containing 10% FBS. After 24 hr, cells were transfected with siRNA (100 nM) using Lipofectamine RNAiMAX according to the manufacturer's instructions. After 6 hr, media were replaced. TEAD1 (L-012603-00-0005), RIOK2 (L-005002-00-0005), ELP5 (L-017992-00-0005), VRK1 (L-004683-00-0005), WWTR1 (L-016083-00-0005) and ELP3 (L-015940-01-0005) siRNAs were obtained from Horizon Discovery.

CRISPR/Cas9 screens. Two separate batches of each cell line were transduced with the TKOv3 CRISPR KO lentivirus library (18) at a low MOI (˜0.3). Two days post-infection, the media were supplemented with puromycin, and cells were selected for 8 days. Following a recovery phase, a 500× library representation of infected cells from each batch was treated with DMSO (vehicle) or MRTX-849 at twice the IC₅₀ for each line for 8 doubling periods. For combination screens, TN0155 was applied at the IC₅₀ dosage or at 3 mM if the IC₅₀ for the cell line exceeded that value. Screens with TNO155 alone used the same concentration of SHP2i as the combination screens. Upon screen conclusion gDNA was extracted and amplified via PCR, as described (44). The final PCR products were sequenced using an Illumina NovaSeq 6000 (SP 100 Cycle Flow Cell v1.5), and sequencing results were analyzed using MaGeCK (20). Downstream statistical analyses and plot generations were performed in R environment software (4.0.3). Pathway analysis were generated by Enrichr (45). Gene set enrichment analysis was obtained by deploying GSEA software (46). CIRCOS plots were generated by using Metascape (47).

Cell viability and proliferation assays. Cells were seeded in 96-well plates (1,000-2,000 cells/well) and treated with drugs at the indicated concentrations for the indicated times (3-7 days). Media (including inhibitors) were replaced every two days. Dose-response curves were generated using the MTS-based Cell Counting Kit-8 (CCK8) assay (Enzo, ALX-850-039-KI02). Three hours after addition of CCK-8 reagent to cells, A₄₅₀ was recorded using a FlexStation 3 multi-mode microplate reader according to the manufacturer's instructions. IC₅₀s were calculated with GraphPad Prism. For all other proliferation assays, cells were harvested and stained with trypan blue (#T8154, Sigma-Aldrich), and viable (trypan blue-excluding) cells were quantified with a Countless II automated cell counter (Invitrogen). Drug interaction between MRTX-849 and VT104 was assessed by Bliss analysis using the formula: Y_{ab,P}=Yₐ+Y_{b}−YₐY_{b}, where Yₐ stands for percentage inhibition of drug a and Y_{b} stands for percentage inhibition of drug b. Synergy was defined as % observed effect>Y_{ab,P} (48).

RNA extraction and RT-qPCR. Total RNA was extracted from cell pellets using the RNeasy Plus Mini Kit (QIAGEN, Cat #: 74136) and reverse transcribed using the High-Capacity RNA-to-cDNA™ Kit (Thermo Fisher Scientific, Cat #: 4387406) as per the manufacturers' protocols. cDNAs were diluted and analyzed by RT-qPCR using PowerUp™ SYBR™ Green Master Mix (Thermo Fisher Scientific, Cat #: A25742). PCR amplification and detection were achieved by using the QuantStudio 3 Real-Time PCR System (Applied Biosystems) and CYR61-specific primers (F-CYR61: CTCGCCTTAGTCGTCACCC (SEQ ID NO: 1); R-CYR61: CGCCGAAGTTGCATTCCAG (SEQ ID NO: 2). CYR61 levels were normalized to those of ACTB and represented as fold-change in gene expression in the test sample relative to the control.

Bulk RNA-seq. Bulk RNA-seq was performed on total RNA from cell lines or isolated tumor cells by the PCC Genome Technology Center Shared Resource (GTC). Libraries were prepared with the Illumina TruSeq Stranded Total RNA Sample Preparation Kit and sequenced on an Illumina NovaSeq 6000 platform utilizing 150-bp paired end reads. Sequencing data were de-multiplexed and transformed into FASTQ format by using Illumina bcl2fastq software. Subsequent data processing and analysis were performed by the PCC Applied Bioinformatics Laboratories (ABL). Briefly, sequencing results were transformed into FASTQ format by using Illumina bcl2fastq software. Reads were adapter- and quality-trimmed using Trimmomatic before alignment with the human or mouse genome using the splice-aware STAR aligner. Using featureCounts (49), counts for each gene were created based on the number of aligned reads overlapping its exons. These counts were standardized and subsequently evaluated for differential expression via the DESeq2 R package, using negative binomial generalized linear models. Lastly, the Enrichr tool was employed for pathway analysis of the bulk RNA-seq data.

Luciferase assays. H2030 cells in 6-well plates were transiently co-transfected with 8×GTIIC-luciferase (2 mg) and Renilla luciferase (200 ng) plasmids, using X-tremeGENE HP DNA Transfection Reagent (Sigma). Luciferase activity was measured with the Dual-Glo Luciferase Assay System (Promega). To control for transfection efficiency, firefly luciferase activity was normalized to Renilla luciferase readings. Experiments were performed three times.

RHOA activity assays. RHOA activity (RHO-GTP) was quantified by using a G-LISA activation assay kit (Cytoskeleton), following the manufacturer's guidelines. Briefly, cells were lysed using a buffer supplied in the kit, and extracts were transferred to 96-well plates layered with GST-RHOA binding domain fusion protein. After incubation with gentle shaking at 4° C. for 30 min., plates were washed three times with “wash buffer” prior to the addition of “antigen-presenting buffer” (each from Cytoskeleton) containing anti-RHOA primary monoclonal antibody for 30 min., washed 3 times, incubated with horseradish peroxidase (HRP)-linked secondary antibodies and quantified by using a FlexStation 3 multi-mode microplate reader.

Immunofluorescence. Cells (2×10³/well) were seeded in 8-well chamber slides (Nunc Lab-Tek, Thermo Fisher), allowed to grow overnight in complete medium (RPMI with 10% FBS and 1× penicillin/streptomycin), and treated with MRTX-849 for the times indicated. Cells were then fixed in 4% paraformaldehyde for 10 minutes at room temperature, rinsed three times for 5 min. each in PBS, and permeabilized in 0.1% Triton X-100/5% FBS in PBS for a 1 hr at RT. Following blocking, slides were incubated with monoclonal anti-YAP1 antibody (Cell Signaling, Cat #14074, 1:100 in 5% FBS/PBS) at 4° C. overnight, washed four times with PBS, incubated with Alexa Fluor Plus 488 (Thermo Fisher Scientific, Cat #: A-11001, 1:400 dilution) secondary antibodies for 2 hr, and washed three times with PBS. Nuclei were stained with DAPI (BioLegend, Cat #: 422801) for 5 min, and slides were washed two more times in PBS before mounting with Fluorescence Mounting Medium (Dako, Cat #: S3023). Images were acquired with a Zeiss 880 Laser Scanning Confocal Microscope (Axio Observer) equipped with Zen 3.0 software (ZEN blue, Carl Zeiss Inc.) and were processed by Fiji software (NIH).

Immunoblotting. Cells were washed twice with ice-cold PBS, scraped into 700 ml PBS, and centrifuged at 1500× r.p.m. for 5 min. Pellets were lysed in RIPA buffer (ThermoFisher Scientific #89900) with a protease and phosphatase inhibitor cocktail (ThermoFisher Scientific #78440). Protein concentrations were determined using a BCA protein assay kit (ThermoFisher Scientific #23225) with bovine serum albumin as the protein standard. Total cellular protein (30 g) was boiled in 6×SDS sample buffer (Boston BioProducts cat #BP-111R), resolved on 4-20% Mini-PROTEAN TGX SDS-PAGE gels (Bio-Rad, Cat #4568095, 5678095 and 5671094), and transferred to nitrocellulose. Membranes were blocked with TBS buffer (LI-COR, Cat #927-60001), incubated with primary antibodies overnight at 4° C. and secondary antibodies for 1 hour at room temperature, and visualized by using an Odyssey classic infrared imaging system (LI-COR) and Image Studio Lite (V 5.2).

Antibodies used for immunoblots were as follows: monoclonal anti-3-actin (#A5441; 1:10,000) and anti-FLAG (#F1804; 1:5,000) from Sigma; monoclonal anti-3-actin (#4970S), anti-GAPDH (#5174S; 1:5000), anti-TEAD1 (#12292; 1:1000), anti-ELP3 (#5728S; 1:1000), anti-TAZ (#72804; 1:1000), and monoclonal anti-GFP (#2956; 1:1000), all from Cell Signaling; polyclonal anti-RIOK2 (Abcam #Ab88485; 1:1000), polyclonal anti-ELP5 (Protein Tech #10162-1-AP; 1:500), and IRDye 680RD donkey anti-Mouse IgG (#925-68072), IRDye 800CW donkey anti-rabbit IgG (#925-32212), IRDye 680RD donkey anti-Rabbit IgG (#926-68073), and IRDye 800CW donkey anti-Mouse IgG (#925-32212), all from LICOR.

Genetically engineered mouse model (GEMM) generation and treatment. All animal studies were approved by the Institutional Animal Care and Use Committee at New York University Grossman School of Medicine and adhered to the guidelines stipulated in the Guide for the Care and Use of Laboratory Animals. KRAS^{LSL-G12D/+}; Stk11^{flox/flox} (KdL) (50) and KRAS^{LSL-G12C/+} (KC) (51) mice were described previously. These mice were inter-crossed to generate KRAS^{LSL-G12C/+};Stk11^{flox/flox} (KCL) progeny, all on C57BL6/J background. Mice of both sexes were used for experiments, and age- and sex-matched animals were grouped randomly. Ad-Cre virus (1×10⁷ PFU) was instilled nasally at 7-8 weeks of age, and mice were monitored for tumor development by magnetic resonance imaging (MRI). KCL tumor-bearing mice were dosed by gavage with MRTX-849 (100 mg/kg), alone or in combination with SHTP099 (75 mg/kg), on a 5 days on/2 days off schedule. Tumor development was monitored by MRI every 2 weeks (see below). After 3-6 months of treatment, tumors were harvested and cut into pieces for RNA-seq and RPPA analysis.

For syngeneic tumor experiments, KCL cells (10⁶ in 200 ml PBS) were injected subcutaneously into the right flanks of male C57BL/6J mice (Jackson Laboratory). For xenografts, H2030 cells (5×10⁶) were injected subcutaneously into the right flanks of Crl:NU-Foxn1nu/nu mice (Charles River, #088) with a 1:1 mixture of cell suspension in Matrigel (Corning, Cat #354234). When tumor volumes reached ˜100 mm³ (for syngeneic grafts) or ˜300 mm³ (for xenografts), mice were randomized to the following groups: vehicle control, MRTX-849 (100 mg/kg/d), VT104 (10 mg/kg/d), and MRTX-849 (100 mg/kg/d) and VT104 (10 mg/kg/d). Mice were weighed two times a week prior to dosing and throughout the study. Tumors were measured in 2 dimensions (length and width) twice a week and volumes (mm³) were calculated as (length×width²)/2).

MRI quantification. For magnetic resonance imagining (MRI) of the lung fields, mice were anesthetized with isoflurane, and 16 consecutive sections were scanned using a BioSpec USR70/30 horizontal bore system (Bruker). Tumor volumes in whole lungs were quantified using 3-D slicer software. Acquisition of MRI signals was adapted to cardiac and respiratory cycles to minimize motion effects during imaging.

scRNA-seq and RPPA of patient samples. Tumor specimens were collected from patients at New York University Langone Hospital (New York, NY) with the approval of the Institutional Review Board, in accordance with the Declaration of Helsinki, CIOMS, Belmont Report, and U.S. Common Rule and signed patient consent. scRNAseq was performed on cells from fresh tumor biopsies from patients with KRAS^{G12C}-mutant NSCLC treated with AMG-510 (sotorasib) or MRTX-849 (adagrasib)/TNO155 or from a control KRAS^{G}V-mutant tumor. Tumor samples were mechanically and enzymatically digested using Collagenase 1000× and DNase for 15-30 min at 37° C. Single cells were passed through a 70 mm filter, and red blood cells were lysed in ACK buffer. Cells were counted with trypan blue, stained with 0.1 uM Calcein (final concentration) for 20 min at 37° C. at a final cell concentration of ˜10⁶ cells/ml, and resuspended in media containing DAPI (1:1000 of 1 mg/mL stock). Single cells in 0.04% BSA solution in PBS were recovered by FACS (PCC Immune Monitoring Laboratory) and subjected to 10× Genomics scRNAseq library prep and sequencing. Raw sequencing files were mapped to the reference genome (hg38), and gene-cell matrices were generated by 10× Genomics Cell Ranger software (v 3.1.0). Matrices from different samples were merged and imported into Seurat (v 4.1.3). Quality controls included calculating the number of genes, UMIs, and the proportion of mitochondrial genes for each cell. Cells with a low number of covered genes (gene count<500) or high mitochondrial counts (mt-genes>0.2) were filtered. Log-normalization was performed on the filtered matrix, Principal Component Analysis (PCA) was performed, and the top 20 PCs were used as input for Uniform Manifold Approximation and Projection (UMAP) and graph-based clustering. Marker genes were used to determine tumor cell types. All downstream statistical analyses and plot generation were performed in R environment (4.0.3).

RPPA was performed at the MDACC Functional Proteomics core facility as described (52). Standardized intensity data were log 2-transformed, and heat maps were row-normalized using Z score. Rows and columns were clustered via hierarchical clustering as described (52).

Statistical analysis. Data are expressed as mean±standard deviation. Significance was assessed using Student's t test, or 1-way ANOVA with Tukey's multiple comparisons test, as appropriate. Statistical analyses were performed in Prism 8 (GraphPad Software). Significance was set at P=0.05 for all analyses except screen data, where genes with FDR<0.1 were considered for further analysis. P values and FDRs for individual experiments are stated in the text and/or figure legends.

### REFERENCES

1. Yaeger R, Chatila W K, Lipsyc M D, Hechtman J F, Cercek A, Sanchez-Vega F, et al. Clinical Sequencing Defines the Genomic Landscape of Metastatic Colorectal Cancer. Cancer Cell 2018; 33:125-36 e3
2. Campbell J D, Alexandrov A, Kim J, Wala J, Berger A H, Pedamallu C S, et al. Distinct patterns of somatic genome alterations in lung adenocarcinomas and squamous cell carcinomas. Nat Genet 2016; 48:607-16
3. Bailey P, Chang D K, Nones K, Johns A L, Patch A M, Gingras M C, et al. Genomic analyses identify molecular subtypes of pancreatic cancer. Nature 2016; 531:47-52
4. Arbour K C, Jordan E, Kim H R, Dienstag J, Yu H A, Sanchez-Vega F, et al. Effects of Co-occurring Genomic Alterations on Outcomes in Patients with KRAS-Mutant Non-Small Cell Lung Cancer. Clin Cancer Res 2018; 24:334-40
5. Skoulidis F, Byers L A, Diao L, Papadimitrakopoulou V A, Tong P, Izzo J, et al. Co-occurring genomic alterations define major subsets of KRAS-mutant lung adenocarcinoma with distinct biology, immune profiles, and therapeutic vulnerabilities. Cancer Discov 2015; 5:860-77
6. Negrao M V, Araujo H A, Lamberti G, Cooper A J, Akhave N S, Zhou T, et al. Co-mutations and KRAS G12C inhibitor efficacy in advanced NSCLC. Cancer Discov 2023
7. Papillon-Cavanagh S, Doshi P, Dobrin R, Szustakowski J, Walsh A M. STK11 and KEAP1 mutations as prognostic biomarkers in an observational real-world lung adenocarcinoma cohort. ESMO Open 2020; 5
8. Punekar S R, Velcheti V, Neel B G, Wong K K. The current state of the art and future trends in RAS-targeted cancer therapies. Nat Rev Clin Oncol 2022; 19:637-55
9. Dy G K, Govindan R, Velcheti V, Falchook G S, Italiano A, Wolf J, et al. Long-Term Outcomes and Molecular Correlates of Sotorasib Efficacy in Patients With Pretreated KRAS G12C-Mutated Non-Small-Cell Lung Cancer: 2-Year Analysis of CodeBreaK 100. J Clin Oncol 2023; 41:3311-7
10. Janne P A, Riely G J, Gadgeel S M, Heist R S, Ou S I, Pacheco J M, et al. Adagrasib in Non-Small-Cell Lung Cancer Harboring a KRAS(G12C) Mutation. N Engl J Med 2022; 387:120-31
11. Falchook G, Li B T, Marrone K A, Bestvina C M, Langer C J, Krauss J C, et al. OA03.03 Sotorasib in Combination with RMC-4630, a SHP2 Inhibitor, in KRAS p.G12C-Mutated NSCLC and Other Solid Tumors. Journal of Thoracic Oncology 2022; 17:S8
12. Skoulidis F, Li B T, Dy G K, Price T J, Falchook G S, Wolf J, et al. Sotorasib for Lung Cancers with KRAS p.G12C Mutation. N Engl J Med 2021; 384:2371-81
13. de Langen A J, Johnson M L, Mazieres J, Dingemans A C, Mountzios G, Pless M, et al. Sotorasib versus docetaxel for previously treated non-small-cell lung cancer with KRAS(G12C) mutation: a randomised, open-label, phase 3 trial. Lancet 2023; 401:733-46
14. Weiss A, Lorthiois E, Barys L, Beyer K S, Bomio-Confaglia C, Burks H, et al. Discovery, Preclinical Characterization, and Early Clinical Activity of JDQ443, a Structurally Novel, Potent, and Selective Covalent Oral Inhibitor of KRASG12C. Cancer Discov 2022; 12:1500-17
15. Ryan M B, Coker O, Sorokin A, Fella K, Barnes H, Wong E, et al. KRAS(G12C)-independent feedback activation of wild-type RAS constrains KRAS(G12C) inhibitor efficacy. Cell Rep 2022; 39:110993
16. Skoulidis F, Arbour K C, Hellmann M D, Patil P D, Marmarelis M E, Awad M M, et al. Association of STK11/LKB1 genomic alterations with lack of benefit from the addition of pembrolizumab to platinum doublet chemotherapy in non-squamous non-small cell lung cancer. Journal of Clinical Oncology 2019; 37:102-
17. West H J, McCleland M, Cappuzzo F, Reck M, Mok T S, Jotte R M, et al. Clinical efficacy of atezolizumab plus bevacizumab and chemotherapy in KRAS-mutated non-small cell lung cancer with STK11, KEAP1, or TP53 comutations: subgroup results from the phase III IMpower150 trial. Journal for Immunotherapy of Cancer 2022; 10
18. Hart T, Tong A H Y, Chan K, Van Leeuwen J, Seetharaman A, Aregger M, et al. Evaluation and Design of Genome-Wide CRISPR/SpCas9 Knockout Screens. G3 (Bethesda) 2017; 7:2719-27
19. Mair B, Tomic J, Masud S N, Tonge P, Weiss A, Usaj M, et al. Essential Gene Profiles for Human Pluripotent Stem Cells Identify Uncharacterized Genes and Substrate Dependencies. Cell Rep 2019; 27:599-615 e12
20. Li W, Xu H, Xiao T, Cong L, Love M I, Zhang F, et al. MAGeCK enables robust identification of essential genes from genome-scale CRISPR/Cas9 knockout screens. Genome Biol 2014; 15:554
21. Sulahian R, Kwon J J, Walsh K H, Pailler E, Bosse T L, Thaker M, et al. Synthetic Lethal Interaction of SHOC2 Depletion with MEK Inhibition in RAS-Driven Cancers. Cell Rep 2019; 29:118-34 e8
22. Kim D, Xue J Y, Lito P. Targeting KRAS(G12C): From Inhibitory Mechanism to Modulation of Antitumor Effects in Patients. Cell 2020; 183:850-9
23. Brooks A N, Kilgour E, Smith P D. Molecular pathways: fibroblast growth factor signaling: a new therapeutic opportunity in cancer. Clin Cancer Res 2012; 18:1855-62
24. Pobbati A V, Kumar R, Rubin B P, Hong W. Therapeutic targeting of TEAD transcription factors in cancer. Trends Biochem Sci 2023; 48:450-62
25. Nguyen C D K, Yi C. YAP/TAZ Signaling and Resistance to Cancer Therapy. Trends Cancer 2019; 5:283-96
26. Yuan Y, Park J, Feng A, Awasthi P, Wang Z, Chen Q, et al. YAP1/TAZ-TEAD transcriptional networks maintain skin homeostasis by regulating cell proliferation and limiting KLF4 activity. Nat Commun 2020; 11:1472
27. Rosenbluh J, Nijhawan D, Cox A G, Li X, Neal J T, Schafer E J, et al. beta-Catenin-driven cancers require a YAP1 transcriptional complex for survival and tumorigenesis. Cell 2012; 151:1457-73
28. Zhao B, Ye X, Yu J, Li L, Li W, Li S, et al. TEAD mediates YAP-dependent gene induction and growth control. Genes Dev 2008; 22:1962-71
29. Franklin J M, Wu Z, Guan K L. Insights into recent findings and clinical application of YAP and TAZ in cancer. Nat Rev Cancer 2023
30. Piccolo S, Panciera T, Contessotto P, Cordenonsi M. YAP/TAZ as master regulators in cancer: modulation, function and therapeutic approaches. Nat Cancer 2023; 4:9-26
31. Konstantinidou G, Ramadori G, Torti F, Kangasniemi K, Ramirez R E, Cai Y, et al. RHOA-FAK is a required signaling axis for the maintenance of KRAS-driven lung adenocarcinomas. Cancer Discov 2013; 3:444-57
32. Yu F X, Zhao B, Guan K L. Hippo Pathway in Organ Size Control, Tissue Homeostasis, and Cancer. Cell 2015; 163:811-28
33. Totaro A, Panciera T, Piccolo S. YAP/TAZ upstream signals and downstream responses. Nat Cell Biol 2018; 20:888-99
34. Fedele C, Li S, Teng K W, Foster C J R, Peng D, Ran H, et al. SHP2 inhibition diminishes KRASG12C cycling and promotes tumor microenvironment remodeling. J Exp Med 2021; 218
35. Ryan M B, Fece de la Cruz F, Phat S, Myers D T, Wong E, Shahzade H A, et al. Vertical Pathway Inhibition Overcomes Adaptive Feedback Resistance to KRAS(G12C) Inhibition. Clin Cancer Res 2020; 26:1633-43
36. Ahmed T A, Adamopoulos C, Karoulia Z, Wu X, Sachidanandam R, Aaronson S A, et al. SHP2 Drives Adaptive Resistance to ERK Signaling Inhibition in Molecularly Defined Subsets of ERK-Dependent Tumors. Cell Rep 2019; 26:65-78 e5
37. Kerr D L, Haderk F, Bivona T G. Allosteric SHP2 inhibitors in cancer: Targeting the intersection of RAS, resistance, and the immune microenvironment. Curr Opin Chem Biol 2021; 62:1-12
38. Fan M, Lu W, Che J, Kwiatkowski N P, Gao Y, Seo H S, et al. Covalent disruptor of YAP-TEAD association suppresses defective Hippo signaling. Elife 2022; 11
39. Tang T T, Konradi A W, Feng Y, Peng X, Ma M, Li J, et al. Small Molecule Inhibitors of TEAD Auto-palmitoylation Selectively Inhibit Proliferation and Tumor Growth of NF2-deficient Mesothelioma. Mol Cancer Ther 2021; 20:986-98
40. Cordenonsi M, Zanconato F, Azzolin L, Forcato M, Rosato A, Frasson C, et al. The Hippo transducer TAZ confers cancer stem cell-related traits on breast cancer cells. Cell 2011; 147:759-72
41. Serafim R A M, de Souza Gama F H, Dutra L A, Dos Reis C V, Vasconcelos SNS, da Silva Santiago A, et al. Development of Pyridine-based Inhibitors for the Human Vaccinia-related Kinases 1 and 2. ACS Med Chem Lett 2019; 10:1266-71
42. Gaik M, Kojic M, Wainwright B J, Glatt S. Elongator and the role of its subcomplexes in human diseases. EMBO Mol Med 2023; 15:e16418
43. Pan Y, Han H, Hu H, Wang H, Song Y, Hao Y, et al. KMT2D deficiency drives lung squamous cell carcinoma and hypersensitivity to RTK-RAS inhibition. Cancer Cell 2023; 41:88-105 e8
44. Olivieri M, Durocher D. Genome-scale chemogenomic CRISPR screens in human cells using the TKOv3 library. STAR Protoc 2021; 2:100321
45. Kuleshov M V, Jones M R, Rouillard A D, Fernandez N F, Duan Q, Wang Z, et al. Enrichr: a comprehensive gene set enrichment analysis web server 2016 update. Nucleic Acids Res 2016; 44:W90-7
46. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA 2005; 102:15545-50
47. Zhou Y, Zhou B, Pache L, Chang M, Khodabakhshi A H, Tanaseichuk O, et al. Metascape provides a biologist-oriented resource for the analysis of systems-level datasets. Nat Commun 2019; 10:1523
48. Zhao W, Sachsenmeier K, Zhang L, Sult E, Hollingsworth R E, Yang H. A New Bliss Independence Model to Analyze Drug Combination Data. J Biomol Screen 2014; 19:817-21
49. Liao Y, Smyth G K, Shi W. featureCounts: an efficient general purpose program for assigning sequence reads to genomic features. Bioinformatics 2014; 30:923-30
50. Ji H, Ramsey M R, Hayes D N, Fan C, McNamara K, Kozlowski P, et al. LKB1 modulates lung cancer differentiation and metastasis. Nature 2007; 448:807-10
51. Li S, Liu S, Deng J, Akbay E A, Hai J, Ambrogio C, et al. Assessing Therapeutic Efficacy of MEK Inhibition in a KRAS(G12C)-Driven Mouse Model of Lung Cancer. Clin Cancer Res 2018; 24:4854-64
52. Akbani R, Ng P K, Werner H M, Shahmoradgoli M, Zhang F, Ju Z, et al. A pan-cancer proteomic perspective on The Cancer Genome Atlas. Nat Commun 2014; 5:3887
53. Lou K, Steri V, Ge A Y, Hwang Y C, Yogodzinski C H, Shkedi A R, et al. KRAS(G12C) inhibition produces a driver-limited state revealing collateral dependencies. Sci Signal 2019; 12
54. Dey A, Varelas X, Guan K L. Targeting the Hippo pathway in cancer, fibrosis, wound healing and regenerative medicine. Nat Rev Drug Discov 2020; 19:480-94
55. Hagenbeek T J, Zbieg J R, Hafner M, Mroue R, Lacap J A, Sodir N M, et al. An allosteric pan-TEAD inhibitor blocks oncogenic YAP/TAZ signaling and overcomes KRAS G12C inhibitor resistance. Nat Cancer 2023; 4:812-28
56. Adachi Y, Kimura R, Hirade K, Yanase S, Nishioka Y, Kasuga N, et al. Scribble mis-localization induces adaptive resistance to KRAS G12C inhibitors through feedback activation of MAPK signaling mediated by YAP-induced MRAS. Nat Cancer 2023; 4:829-43
57. Mohseni M, Sun J, Lau A, Curtis S, Goldsmith J, Fox V L, et al. A genetic screen identifies an LKB1-MARK signalling axis controlling the Hippo-YAP pathway. Nat Cell Biol 2014; 16:108-17
58. Xu X, Omelchenko T, Hall A. LKB1 tumor suppressor protein regulates actin filament assembly through Rho and its exchange factor Dbl independently of kinase activity. BMC Cell Biol 2010; 11:77
59. Vial E, Sahai E, Marshall C J. ERK-MAPK signaling coordinately regulates activity of Rac1 and RhoA for tumor cell motility. Cancer Cell 2003; 4:67-79
60. Zhao J, Zhou D, Guo J, Ren Z, Zhou L, Wang S, et al. Effect of fasudil hydrochloride, a protein kinase inhibitor, on cerebral vasospasm and delayed cerebral ischemic symptoms after aneurysmal subarachnoid hemorrhage. Neurol Med Chir (Tokyo) 2006; 46:421-8
61. Barcelo J, Samain R, Sanz-Moreno V. Preclinical to clinical utility of ROCK inhibitors in cancer. Trends Cancer 2023; 9:250-63
62. Kim S, Kim S A, Han J, Kim I S. Rho-Kinase as a Target for Cancer Therapy and Its Immunotherapeutic Potential. Int J Mol Sci 2021; 22
63. Shields J A, Meier S R, Bandi M, Mulkearns-Hubert E E, Hajdari N, Ferdinez M D, et al. VRK1 Is a Synthetic-Lethal Target in VRK2-Deficient Glioblastoma. Cancer Res 2022; 82:4044-57
64. So J, Mabe N W, Englinger B, Chow K H, Moyer S M, Yerrum S, et al. VRK1 as a synthetic lethal target in VRK2 promoter-methylated cancers of the nervous system. JCI Insight 2022; 7
65. Cerezo E L, Houles T, Lie O, Sarthou M K, Audoynaud C, Lavoie G, et al. RIOK2 phosphorylation by RSK promotes synthesis of the human small ribosomal subunit. PLoS Genet 2021; 17:e1009583
66. Rapino F, Delaunay S, Rambow F, Zhou Z, Tharun L, De Tullio P, et al. Codon-specific translation reprogramming promotes resistance to targeted therapy. Nature 2018; 558:605-9
67. Cruz-Gordillo P, Honeywell M E, Harper N W, Leete T, Lee M J. ELP-dependent expression of MCL1 promotes resistance to EGFR inhibition in triple-negative breast cancer cells. Sci Signal 2020; 13
68. Khosravi-Far R, Solski P A, Clark G J, Kinch M S, Der C J. Activation of Rac1, RhoA, and mitogen-activated protein kinases is required for Ras transformation. Mol Cell Biol 1995; 15:6443-53
69. Chen J C, Zhuang S, Nguyen T H, Boss G R, Pilz R B. Oncogenic Ras leads to Rho activation by activating the mitogen-activated protein kinase pathway and decreasing Rho-GTPase-activating protein activity. J Biol Chem 2003; 278:2807-18
70. Qiu R G, Chen J, McCormick F, Symons M. A role for Rho in Ras transformation. Proc Natl Acad Sci USA 1995; 92:11781-5

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. (canceled)

2. A method for enhancing sensitivity and/or overcoming or preventing resistance of a KRAS mutant cancer cell to a KRAS inhibitor, comprising inhibiting in said KRAS mutant cancer cell expression or function of one or more proteins selected from VRK1, RIOK2, EXT1, EXT2, ELP2, ELP3, ELP5, PKN2, ROCK1, ROCK2, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2.

3. The method of claim 2, comprising inhibiting in said KRAS mutant cancer cell expression or function of one or more proteins selected from TFIIIC, GTF3C1, TBP, HSD17B10, POP5, RPP21, RTCB, TSEN2, URM1, ELP2, ELP4, ELP1 (IKBKAP), ADAT3, MOCS3, KTI12, ELP3, ELP5, IARS, YARS, SEPSECS, PARS2, YARS2, DARS2, and LARS2.

4. The method of claim 2, comprising inhibiting in said KRAS mutant cancer cell expression or function of one or more proteins selected from VRK1, RIOK2, PKN2, ROCK1, and ROCK2.

5. (canceled)

6. The method of claim 2, comprising inhibiting in said KRAS mutant cancer cell expression or function of one or more proteins selected from VRK1, ELP2, ELP3, PKN2, RIOK2, EXT1, and EXT2.

7. The method of claim 6, comprising inhibiting in said KRAS mutant cancer cell expression or function of VRK1 protein, PKN2 protein, and/or RIOK2 protein.

8. The method of claim 7, comprising inhibiting in said KRAS mutant cancer cell kinase activity of VRK1 protein, RIOK2 protein, and/or PKN2 protein, and/or ATPase activity of RIOK2 protein.

9. (canceled)

10. The method of claim 2, comprising administering to said KRAS mutant cancer cell an inhibitor of expression or function of said one or more proteins or a degrader of said one or more proteins.

11. (canceled)

12. The method of claim 10, wherein the method comprises administering to said KRAS mutant cancer cell an inhibitor of expression or function or degrader of ROCK1 protein and/or ROCK2 protein.

13. The method of claim 12, wherein the inhibitor of expression or function or degrader of ROCK1 protein and/or ROCK2 protein is selected from AR-12286, Fasudil, Ripasudil, Netarsudil, KD025 (Belumosudil), AT13148, GSK269962, H1152, GSK429286, pharmaceutically acceptable salts thereof, and any combinations thereof.

14. The method of claim 2, wherein the KRAS mutant cancer cell comprises a mutation selected from a KRAS G12 mutation, a KRAS G13 mutation, a KRAS H61 mutation, and a KRAS K117 mutation.

15. The method of claim 14, wherein the KRAS mutant cancer cell comprises: i) a KRAS G12 mutation selected from G12C, G12V, G12D, G12S, and G12R; ii) a KRAS G13D mutation: iii) a KRAS H61 mutation selected from Q61H, Q61L, and Q61R; and/or iv) a KRAS K117N mutation.

16. The method of claim 15, wherein the KRAS mutant cancer cell comprises the KRAS G12C mutation.

17. The method of claim 16, wherein the KRAS inhibitor is a KRAS G12C inhibitor (G12Ci).

18.-20. (canceled)

21. The method of claim 2, wherein the KRAS mutant cancer cell also has a mutation in STK11 (LKB1) gene and/or KEAP1 gene.

22. The method of claim 2, wherein the KRAS mutant cancer cell also has deletion or reduced expression of one or more genes associated with and/or predictive of resistance of the KRAS mutant cancer cell to treatment with the KRAS inhibitor.

23. (canceled)

24. The method of claim 10, wherein said KRAS mutant cancer cell is in a subject and said inhibitor of expression or function of said one or more proteins or said degrader of said one or more proteins is administered to the subject.

25. The method of claim 2, wherein the KRAS inhibitor is selected from adagrasib (MRTX-849), sotorasib (AMG510), divarasib (GDC-6036), MRTX1133, ARS1620, BI-1701963, N—((R)-1-(3-amino-5-(trifluoromethyl)phenyl)-ethyl)-7-methoxy-2-methyl-6-(((S)-tetrahydrofuran-3-yl)oxy)quinazolin-4-amine, compound 0375-0604, LY3537982, (3S,4S)-8-(6-amino-5-((2-amino-3-chloroyridin-4-yl)thio)pyrazin-2-yl)-3-methyl)2-oxa-8-azaspiro[4.5]decan-4-amine, or (S)-1-(4-(6-chloro-8-fluoro-7-(2-fluoro-6-hydroxyphenyl)quinazolin-4-yl)piperazin-1-yl)prop-2-en-1-one, ARS-3248/JNJ-74699157, JDQ443, MK1084, Compound B, LY3499446, ARS-853, ARS-1620, BI-2852, BI-1823911, BAY-293, BI-2493, BI-2865, RMC6291, RM-018, ASP3082, LC-2, JAB-21822, JAB-23400, D-1553, AZD4625, JNJ-74699157 (ARS-3248), BBO-8520, FMC-376, G12D inhibitor, RAS(On)inhibitors, BBP-454, RMC6236, pharmaceutically acceptable salts thereof, and any combinations thereof.

26. The method of claim 25, wherein the KRAS inhibitor is KRAS G12C inhibitor (G12Ci) adagrasib (MRTX-849) or sotorasib (AMG510).

27. A method of treating a KRAS mutant cancer in a subject in need thereof, comprising administering to the subject an effective amount of a KRAS inhibitor and an inhibitor of expression or function or a degrader of one or more proteins selected from VRK1, RIOK2, PKN2, EXT1, EXT2, ELP2, ELP3, ELP5, ROCK1, ROCK2, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2.

28.-58. (canceled)

59. A pharmaceutical composition comprising (i) a KRAS inhibitor, (ii) an inhibitor of expression or function or a degrader of one or more proteins selected from VRK1, RIOK2, PKN2, EXT1, EXT2, ELP2, ELP3, ELP5, ROCK1, ROCK2, TFIIIC, GTF3C1, TBP, HSD17B10, POP5, RPP21, RTCB, TSEN2, URM1, ELP4, ELP1 (IKBKAP), ADAT3, MOCS3, KTI12, IARS, YARS, SEPSECS, PARS2, YARS2, DARS2, and LARS2, and (iii) a pharmaceutically acceptable carrier and/or excipient.

60.-125. (canceled)


SEQUENCE LISTING (2 sequences)

SEQ ID NO: 1 - DNA, 19 bp, synthetic construct
  source 1..19: mol_type = other DNA
ctcgccttagtcgtcaccc

SEQ ID NO: 2 - DNA, 19 bp, synthetic construct
  source 1..19: mol_type = other DNA
cgccgaagttgcattccag